(19) United States
(12) Patent Application Publication
(10) Pub. No.: US 2026/0015373 A1
(43) Pub. Date: Jan. 15, 2026

(54) STAT6 MODULATORS AND USES THEREOF

(71) Applicant: Recludix Pharma, Inc., San Diego, CA (US)

(72) Inventors: Howard BREGMAN, Melrose, MA (US); John YEOMAN, Medford, MA (US); Jennifer DOWNING, Clinton, MA (US); Samuel K. REZNIK, Cambridge, MA (US); Ernest Allen SICKMIER, Needham, MA (US); Giovanni CIANCHETTA, Boxford, MA (US); Rishi G. VASWANI, Lexington, MA (US); Neil BIFULCO, Sudbury, MA (US); Xia TIAN, Cambridge, MA (US); Andrew TASKER, Simi Valley, CA (US); Brian HODOUS, Arlington, MA (US)

(21) Appl. No.: 19/267,357

(22) Filed: Jul. 11, 2025

Related U.S. Application Data
(60) Provisional application No. 63/670,686, filed on Jul. 12, 2024.

(51) Int. Cl. C07F 9/6561; A61K 31/675

(52) U.S. Cl. CPC C07F 9/6561; A61K 31/675

(57) ABSTRACT

The present disclosure relates generally to STAT6 modulators and uses thereof, and more specifically to compounds, salts, and compositions thereof useful for treating conditions associated with STAT6.

### CROSS REFERENCE TO RELATED APPLICATIONS

This application claims priority benefit to U.S. Provisional Application Nos. 63/670,686, filed Jul. 12, 2024, and 63/743,999, filed Jan. 10, 2025, the disclosures of each of which are hereby incorporated herein by reference in their entireties for all purposes.

### FIELD

The present disclosure relates generally to STAT6 modulators and uses thereof, and more specifically to compounds, salts, and compositions thereof useful for treating conditions associated with STAT6.

### BACKGROUND

The Signal Transducer and Activator of Transcription (STAT) family of proteins consists of transcription factors that play an essential role in the regulation of cell processes, such as proliferation, differentiation, apoptosis and angiogenesis. Seven STAT genes have been identified in the human genome: STAT1, STAT2, STAT3, STAT4, STAT5a, STAT5b, and STAT6.

Recent studies have shown that STAT6 signaling is essential for IL-4- and IL-13-induced epithelial mesenchymal transition (EMT) and aggressiveness of colorectal cancer cells (CRC) cells. STAT6 is involved in several aspects of inflammatory disease and other related conditions.

Given their role in the regulation of cell processes, modulating the activity of one or more STAT proteins, particularly STAT6, represent a pivotal area of investigation for the treatment of cancer, inflammatory conditions, and other therapeutic needs. Therefore, there is a substantial need to supply modulators of STAT, particularly STAT6 modulators.

### BRIEF SUMMARY

The present disclosure provides compounds of Formula (I), compositions thereof, and methods of using these compounds and compositions thereof for the treatment of diseases or conditions associated with STAT, in particular STAT6.

In one aspect, provided is a compound of Formula (I):

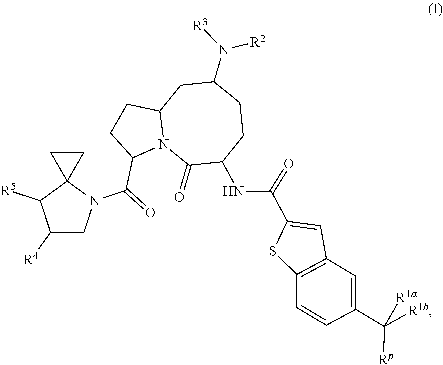

or a pharmaceutically acceptable salt thereof, wherein: R^{1a} and R^{1b} are each independently H or halogen; R² is H, (C₁-C₆)alkyl, or (C₃-C₆)cycloalkyl; R³ is (C₁-C₆)alkyl, halo(C₁-C₆)alkyl, (C₃-C₆)cycloalkyl, 3- to 7-membered heterocyclyl, —(C₁-C₆)alkylene-(C₃-C₆)cycloalkyl, or —(C₁-C₆)alkylene-(3- to 7-membered heterocyclyl), wherein (C₁-C₆)alkyl, (C₃-C₆)cycloalkyl, (C₃-C₆)cycloalkyl of —(C₁-C₆)alkylene-(C₃-C₆)cycloalkyl, or (3- to 7-membered heterocyclyl) of —(C₁-C₆)alkylene-(3- to 7-membered heterocyclyl) is each substituted with 0, 1, 2, 3, 4, or 5 substituents each independently selected from the group consisting of (C₁-C₆)alkyl, (C₁-C₆)alkoxy, cyano, halogen, halo(C₁-C₆)alkyl, and halo(C₁-C₆)alkoxy, or R² and R³ together with the nitrogen atom to which they are attached form 5- to 7-membered heterocyclyl substituted with 0, 1, 2, or 3 substituents each independently selected from the group consisting of (C₁-C₆)alkyl, (C₁-C₆)alkoxy, cyano, halogen, halo(C₁-C₆)alkyl, and halo(C₁-C₆)alkoxy; R⁴ is H, (C₆-C₁₀)aryl, or 5- to 10-membered heteroaryl, wherein (C₆-C₁₀)aryl or 5- to 10-membered heteroaryl is each substituted with 0, 1, 2, or 3 substituents each independently selected from the group consisting of halogen, cyano, (C₁-C₆)alkyl, (C₁-C₆)alkoxy, halo(C₁-C₆)alkyl, halo(C₁-C₆)alkoxy, and —NR^{4a}R^{4b}, wherein: each R^{4a} and R^{4b} is independently H or (C₁-C₆)alkyl; R⁵ is H, cyano, or (C₁-C₆)alkoxy; and R^{P} is phosphonic acid, phosphonate, phosphonamidate, or phosphondiamidate.

### DETAILED DESCRIPTION

The following description is presented to enable a person of ordinary skill in the art to make and use the various embodiments. Descriptions of specific methods, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### Definitions

As used in the present specification, the following words and phrases are generally intended to have the meanings as set forth below, except to the extent that the context in which they are used indicates otherwise.

Throughout this application, unless the context indicates otherwise, references to a compound of Formula (I) include all subgroups defined herein, such as Formula (I-A-1), (I-A-2), (I-A-3), (I-A-4), (I-A-5), (I-A-6), (I-B-1), (I-B-2), (I-B-3), (I-B-4), (I-B-5), (I-B-6), (I-C-1), (I-C-2), (I-C-3), (I-C-4), (I-C-5), (I-C-6), (I-D-1), (I-D-2), (I-D-3), (I-D-4), (I-D-5), (I-D-6), (I-E-1), (I-E-2), (I-E-3), (I-E-4), (I-E-5), (I-E-6), (I-F-1), (I-F-2), (I-F-3), (I-F-4), (I-F-5), (I-F-6), (I-G-1), (I-G-2), (I-G-3), (I-G-4), (I-G-5), (I-G-6), (I-H-1), (I-H-2), (I-H-3), (I-H-4), (I-H-5), (I-H-6), (I-J-1), (I-J-2), (I-J-3), (I-J-4), (I-J-5), or (I-J-6) including all substructures, subgenera, preferences, embodiments, examples, and particular compounds defined and/or described herein. In some embodiments, references to a compound of Formula (I) and subgroups thereof include ionic forms, stereoisomers, rotamers, tautomers, oxides (e.g., N-oxides, S-oxides), esters, prodrugs, isotopologues, and/or protected forms thereof.

When used in connection to describe a chemical group that may have multiple points of attachment, a hyphen (-) designates the point of attachment(s) of that group. For example, —NHC(O)OH means that the point of attachment for this group occurs on the nitrogen atom.

As used herein, “C_{X}-C_{Y}” or “(Cₓ-C_{y})” in reference to or preceding the name of a chemical group (e.g., alkyl, alkoxy, cycloalkyl, aryl) refers to the group having from X to Y carbon atoms, for example a (C₁-C₆)alkyl refers to an alkyl having 1, 2, 3, 4, 5, or 6 carbon atoms. The terms “halo” and “halogen” refer to an atom selected from fluorine (fluoro, —F), chlorine (chloro, —Cl), bromine (bromo, —Br), and iodine (iodo, —I).

Unless otherwise specified, the term “alkyl” when used alone or as part of a larger moiety, such as “haloalkyl”, and the like, means saturated straight-chain or branched monovalent hydrocarbon radical. For example, “(C₁-C₆)alkyl” includes methyl, ethyl, propyl, isopropyl, n-butyl, 1-methylpropyl, isobutyl, tert-butyl, pentyl, isopentyl, neopentyl, and hexyl. The term “alkylene” when used alone or as part of a larger moiety, such as “alkylene-cycloalkyl”, and the like, means saturated straight-chain or branched divalent hydrocarbon radical, for example, —CH₂—, —CH₂CH₂—, —CH₂CH₂CH₂—, —CH₂CH₂CH₂CH₂—, —CH(CH₃)—, and —C(CH₃)₂—.

The term “haloalkyl” includes mono, poly, and perhaloalkyl groups where the halogens are independently selected from fluorine, chlorine, bromine, and iodine. In some embodiments, the halogen is fluorine. For example, haloalkyl includes chloromethyl, dichloromethyl, trichloromethyl, fluoromethyl, difluoromethyl, trifluoromethyl, 1,2-difluoroethyl, 2,2,2-trifluoroethyl, and 1,1,2,2-tetrafluoroethyl.

“Alkoxy” means an alkyl radical attached through an oxygen linking atom, represented by —O-alkyl. For example, “(C₁-C₄)alkoxy” includes methoxy, ethoxy, proproxy, and butoxy.

“Haloalkoxy” is a haloalkyl group which is attached to another moiety via an oxygen atom such as, e.g., —OCHF₂ or —OCF₃.

The term “oxo” means the group ═O.

The term “imino” means the group ═NH.

Unless otherwise specified, the term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic. Monocyclic heteroaryl includes, for example, pyridyl, pyridazinyl, pyrimidinyl, pyrazinyl, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, etc. Bi-cyclic heteroaryls include groups in which a monocyclic heteroaryl ring is fused to one or more aryl or heteroaryl rings. Nonlimiting examples include indolyl, benzooxazolyl, benzooxodiazolyl, indazolyl, benzimidazolyl, benzthiazolyl, benzothiopheneyl, quinolinyl, quinazolinyl, quinoxalinyl, pyrrolopyridinyl, pyrrolopyrimidinyl, pyrrolopyridinyl, thienopyridinyl, thienopyrimidinyl, indolizinyl, purinyl, cinnolinyl, naphthyridinyl, and pteridinyl. It will be understood that when specified, optional substituents on a heteroaryl group may be present on any substitutable position and, include, e.g., the position at which the heteroaryl is attached (where valency permits).

Unless otherwise specified, the term “heterocyclyl” means a 4- to 12-membered saturated or partially unsaturated heterocyclic ring containing 1 to 4 heteroatoms independently selected from N, O, and S. The terms “heterocycle”, “heterocyclyl”, “heterocyclyl ring”, “heterocyclic group”, “heterocyclic moiety”, and “heterocyclic radical”, are used interchangeably herein. A heterocyclyl ring can be attached to its pendant group at any heteroatom or carbon atom that results in a stable structure. A heterocyclyl group may be mono- or bicyclic (e.g., a bridged, fused, or spiro bicyclic ring). Examples of monocyclic saturated or partially unsaturated heterocyclic radicals include, without limitation, azetidinyl, tetrahydrofuranyl, tetrahydrothienyl, terahydropyranyl, pyrrolidinyl, pyrrolidonyl, piperidinyl, oxazolidinyl, piperazinyl, dioxanyl, dioxolanyl, morpholinyl, dihydrofuranyl, dihydropyranyl, dihydropyridinyl, tetrahydropyridinyl, dihydropyrimidinyl, tetrahydropyrimidinyl, dihydrooxadizolyl, and dihydroisoxazolyl. Bi-cyclic heterocyclyl groups include, e.g., unsaturated heterocyclic radicals fused to another unsaturated heterocyclic radical, cycloalkyl, aryl, or heteroaryl ring, such as for example, benzodioxolyl, dihydrobenzodioxinyl, dihydrobenzofuranyl, dihydrobenzothiophenyl, 5-oxa-2,6-diazaspiro[3.4]oct-6-enyl, 6-thia-2,7-diazaspiro[3.4]octanyl, 2,6-diazaspiro[3.3]heptanyl, spiro[indoline-3,3′-pyrrolidine]-yl, thiochromanyl, and the like. It will be understood that when specified, optional substituents on a heterocyclyl group may be present on any substitutable position and, include, e.g., the position at which the heterocyclyl is attached (where valency permits).

The term “spiro” refers to two rings that shares one ring atom (e.g., carbon).

The term “fused” refers to two rings that share two adjacent ring atoms with one another.

The term “bridged” refers to two rings that share three adjacent ring atoms with one another.

The term “aryl” refers to an aromatic carbocyclic single ring or two fused ring system containing 6 to 10 carbon atoms. Examples include phenyl, indanyl, tetrahydronaphthalene, and naphthyl. In some embodiments, the aryl is phenyl or naphthyl. In some embodiments, the aryl is phenyl.

The term “cycloalkyl”, used alone or as part of a larger moiety, refers to a saturated cyclic aliphatic monocyclic or bicyclic ring system, including spirocyclic ring system that may be referred to as “spirocycloalkyl”, having from, unless otherwise specified, 3 to 10 carbon ring atoms. Monocyclic cycloalkyl groups include, without limitation, cyclopropyl, cyclobutyl, cyclopentyl, cyclopentenyl, cyclohexyl, cyclohexenyl, cycloheptyl, cycloheptenyl, and cyclooctyl. It will be understood that when specified, optional substituents on a cycloalkyl or cycloaliphatic group may be present on any substitutable position and, include, e.g., the position at which the cycloalkyl group is attached.

The term “N-linked amino acid” refers to an amino acid that is attached to the indicated moiety via the main-chain amino group. For example, N-linked alanine can be represented by —NHCH(CH₃)C(O)OH. N-linked amino acids can be substituted or unsubstituted.

The term “N-linked amino acid ester” refers to an N-linked amino acid where the main-chain carboxylic acid group and/or any other carboxylic acid group(s) has been converted to an ester group. N-linked amino acid esters can be substituted or unsubstituted.

The term “amino acid” refers to any amino acid (both standard and non-standard amino acids, and both natural and non-natural amino acids), including, but not limited to, α-amino acids, 3-amino acids, γ-amino acids, and 6-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan, and valine. Additional examples of suitable amino acids include, but are not limited to, ornithine, hypusine, 2-aminoisobutyric acid, dehydroalanine, gamma-aminobutyric acid, citrulline, beta-alanine, alpha-ethyl-glycine, alpha-propyl-glycine and norleucine.

Non-natural amino acids are known in the art and include, e.g., alpha-alkyl amino acids (e.g., alpha methyl), alpha-alkylalkoxy amino acids (e.g., alpha —CH₂OCH₃), N-methyl amino acids, homo-amino acids, etc.

The term “phosphonic acid,” as used herein, refers to an organophosphorous group containing a P(═O) moiety where the phosphorous atom is bonded to a carbon atom (herein the point of attachment of R^{P}) and two hydroxy groups, i.e., —P(═O)(OH)₂. The term “phosphonate,” as used herein, refers to an organophosphorous group containing a P(═O) moiety where the phosphorous atom is bonded to a carbon atom (herein the point of attachment of R^{P}) and two monovalent oxygen-linked groups, up to one of which may be hydroxy, and the other or both of which may be a non-hydroxy group, such as alkyloxy, aryloxy, cycloalkyloxy, heteroaryloxy, or heterocyclyloxy. The term “phosphonamidate,” as used herein, refers to an organophosphorous group containing a P(═O) moiety where the phosphorous atom is bonded to a carbon atom (herein the point of attachment of R^{P}), one monovalent oxygen-linked group, such as hydroxy, alkyloxy, aryloxy, cycloalkyloxy, heteroaryloxy, or heterocyclyloxy, and one monovalent nitrogen-linked group, such as alkylamino, arylamino, cycloalkylamino, heteroarylamino, heterocyclylamino, N-linked amino acid, N-linked amino acid ester, N-linked heteroaryl, or N-linked heterocyclyl. The term “phosphondiamidate,” as used herein, refers to an organophosphorous group containing a P(═O) moiety where the phosphorous atom is bonded to a carbon atom (herein the point of attachment of R^{P}) and two monovalent nitrogen-linked group, such as alkylamino, arylamino, cycloalkylamino, heteroarylamino, heterocyclylamino, N-linked amino acid, N-linked amino acid ester, N-linked heteroaryl, or N-linked heterocyclyl.

The term “substituted” refers to one or more hydrogen radical of the designated group being replaced with the radical(s) of a moiety other than hydrogen. Unless otherwise noted, the substituent(s) can be in any position(s), provided that the respective compound is sufficiently stable and pharmaceutically acceptable. Reference to a group being substituted by, for example, 0, 1, 2, or 3 substituents indicates the group is optionally substituted, that is the group may be unsubstituted (have zero substituents) or may be substituted with one, two, or three substituents.

Compounds having one or more chiral centers can exist in various stereoisomeric forms. Stereoisomers are compounds that differ only in their spatial arrangement. Stereoisomers include all diastereomeric, enantiomeric, and epimeric forms as well as racemates and mixtures thereof. A “geometric isomer” refers to stereoisomers that differ in the orientation of substituent group in relationship to a carbon-carbon double bond, a cycloalkyl ring, or a bridged bicyclic system. Atoms (other than H) on each side of a carbon-carbon double bond may be in an E (substituents are on opposite sides of the carbon-carbon double bond) or Z (substituents are oriented on the same side) configuration. “Cis” refers to substituents oriented on the same side of a double bond or ring, whereas “trans” refers to substituents oriented on opposite sides of a double bond or ring.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a geometric isomer is depicted by name or structure, the enrichment of the indicated isomer relative to the opposite isomer is greater than 50%, 60%, 70%, 80%, 90%, 99% or 99.9%. “Enrichment of the indicated isomer relative to the opposite isomer” is a mole percent and is determined by dividing the number of compounds with the indicated geometrical configuration by the total number of all of the compounds with the same or opposite geometrical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The term “inhibit,” “inhibition” or “inhibiting” includes a decrease in the baseline activity of a biological activity or process.

The term “pharmaceutically acceptable excipient” refers to a compound suitable for use in contact with recipient animals, particularly mammals, and more particularly humans, and having a toxicity, irritation, or allergic response commensurate with a reasonable benefit/risk ratio, and effective for their intended use.

As used herein, the term “pharmaceutically acceptable salt” refers to salts that are, within the scope of sound medical judgment, suitable for administration to a subject without undue toxicity, irritation, allergic response, or other undesired effect, commensurate with a reasonable benefit/risk ratio. Pharmaceutically acceptable salts of the compounds describe herein include those derived from suitable inorganic and organic acids and bases.

The term “effective amount” or “therapeutically effective amount” refers to an amount of a compound described herein that is sufficient to achieve the desired biological effect. In some embodiments, the effective amount is sufficient to achieve the desired therapeutic effect (such as treatment of a condition recited herein) under the conditions of administration by modulating, e.g., inhibiting STAT, in particular STAT6. The therapeutically effective amount will vary depending on the compound, the disease or condition and its severity and the age, weight, or other characteristics of the subject to be treated.

The term “administer,” “administering,” and “administration,” refer to contacting a subject with a compound or composition, or to prescribing, instructing, managing, or supervising the contacting of a subject with a compound or a composition by the subject or by another.

### Compounds

Compounds and salts thereof (such as pharmaceutically acceptable salts) are detailed herein, including in the Brief Summary and in the appended claims. Also provided are the use of all of the compounds described herein, including any and all stereoisomers, including geometric isomers (e.g., cis/trans, E/Z isomers), enantiomers, diastereomers, and mixtures thereof in any ratio including racemic mixtures, and salts of the compounds described herein, as well as methods of making such compounds. Any compound described herein may also be referred to as a drug.

In one aspect, provided is a compound of Formula (I):

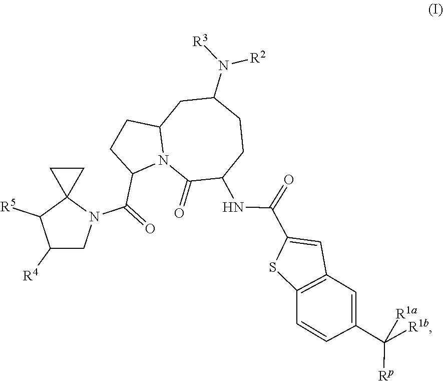

or a pharmaceutically acceptable salt thereof, wherein:
R^{1a} and R^{1b} are each independently H or halogen;
R² is H, (C₁-C₆)alkyl, or (C₃-C₆)cycloalkyl;
R³ is (C₁-C₆)alkyl, halo(C₁-C₆)alkyl, (C₃-C₆)cycloalkyl, 3- to 7-membered heterocyclyl, —(C₁-C₆)alkylene-(C₃-C₆)cycloalkyl, or —(C₁-C₆)alkylene-(3- to 7-membered heterocyclyl), wherein (C₁-C₆)alkyl, (C₃-C₆)cycloalkyl, (C₃-C₆)cycloalkyl of —(C₁-C₆)alkylene-(C₃-C₆)cycloalkyl, or (3- to 7-membered heterocyclyl) of —(C₁-C₆)alkylene-(3- to 7-membered heterocyclyl) is each substituted with 0, 1, 2, 3, 4, or 5 substituents each independently selected from the group consisting of (C₁-C₆)alkyl, (C₁-C₆)alkoxy, cyano, halogen, halo(C₁-C₆)alkyl, and halo(C₁-C₆)alkoxy,
or R² and R³ together with the nitrogen atom to which they are attached form 5- to 7-membered heterocyclyl substituted with 0, 1, 2, or 3 substituents each independently selected from the group consisting of (C₁-C₆)alkyl, (C₁-C₆)alkoxy, cyano, halogen, halo(C₁-C₆)alkyl, and halo(C₁-C₆)alkoxy;
R⁴ is H, (C₆-C₁₀)aryl, or 5- to 10-membered heteroaryl, wherein (C₆-C₁₀)aryl or 5- to 10-membered heteroaryl is each substituted with 0, 1, 2, or 3 substituents each independently selected from the group consisting of halogen, cyano, (C₁-C₆)alkyl, (C₁-C₆)alkoxy, halo(C₁-C₆)alkyl, halo(C₁-C₆)alkoxy, and —NR^{4a}R^{4b}, wherein:
   each R^{4a} and R^{4b} is independently H or (C₁-C₆)alkyl;
R⁵ is H, cyano, or (C₁-C₆)alkoxy; and
R^{P} is phosphonic acid, phosphonate, phosphonamidate, or phosphondiamidate.

In some embodiments, the compound of Formula (I), or a pharmaceutically acceptable salt thereof, is a compound of Formula (I-A-1), (I-A-2), (I-A-3), (I-A-4), (I-A-5), or (I-A-6), or a pharmaceutically acceptable salt thereof:

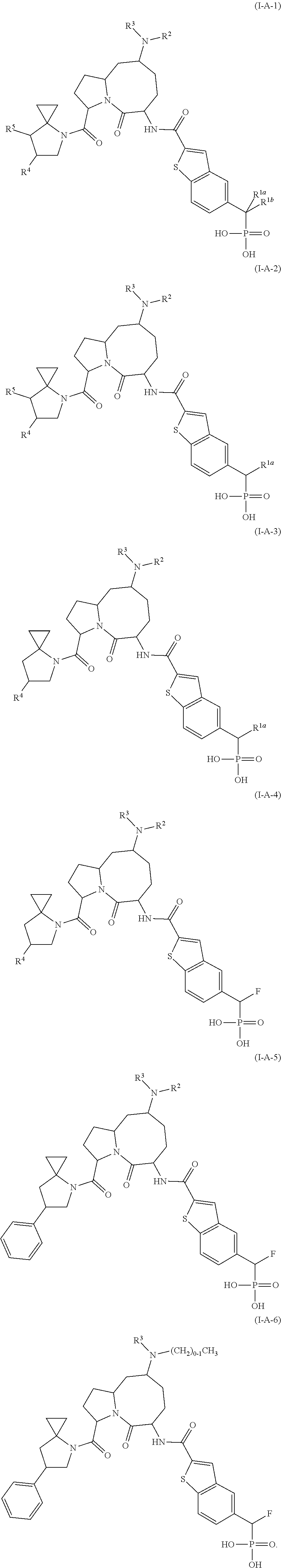

In some embodiments, the compound of Formula (I), or a pharmaceutically acceptable salt thereof, is a compound of Formula (I-B-1), (I-B-2), (I-B-3), (I-B-4), (I-B-5), or (I-B-6), or a pharmaceutically acceptable salt thereof:

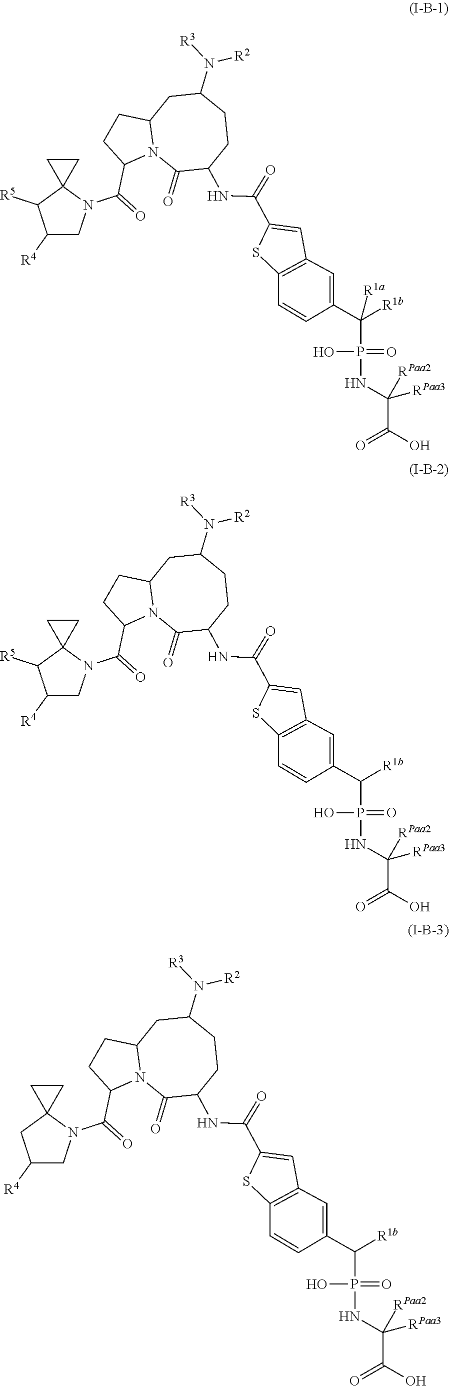

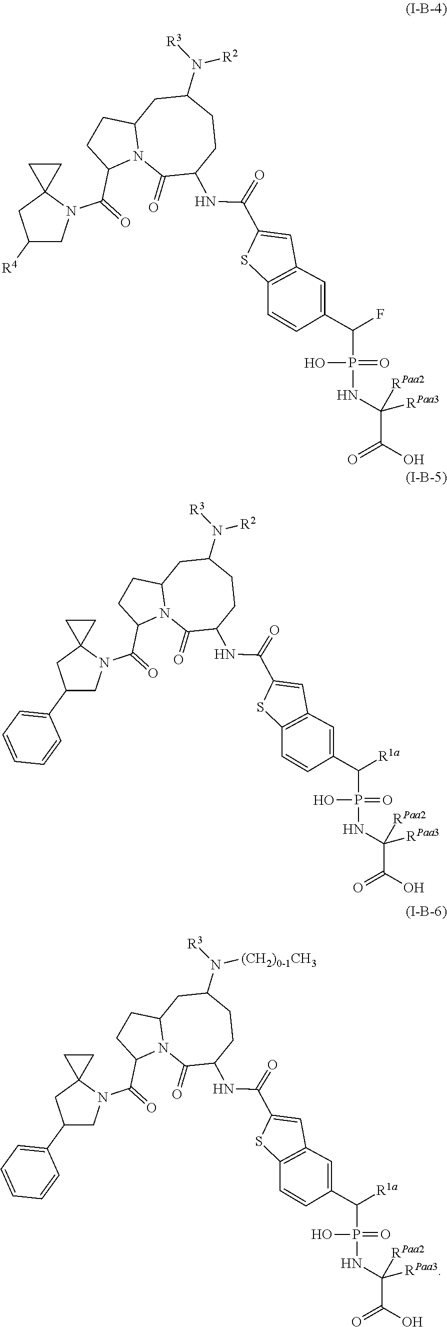

wherein R^{Paa2} is H or (C₁-C₆)alkyl; and R^{Paa2} is H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3} together with the carbon atom to which they are attached form (C₃-C₆)cycloalkyl.

In some embodiments, the compound of Formula (I), or a pharmaceutically acceptable salt thereof, is a compound of Formula (I-C-1), (I-C-2), (I-C-3), (I-C-4), (I-C-5), or (I-C-6), or a pharmaceutically acceptable salt thereof:

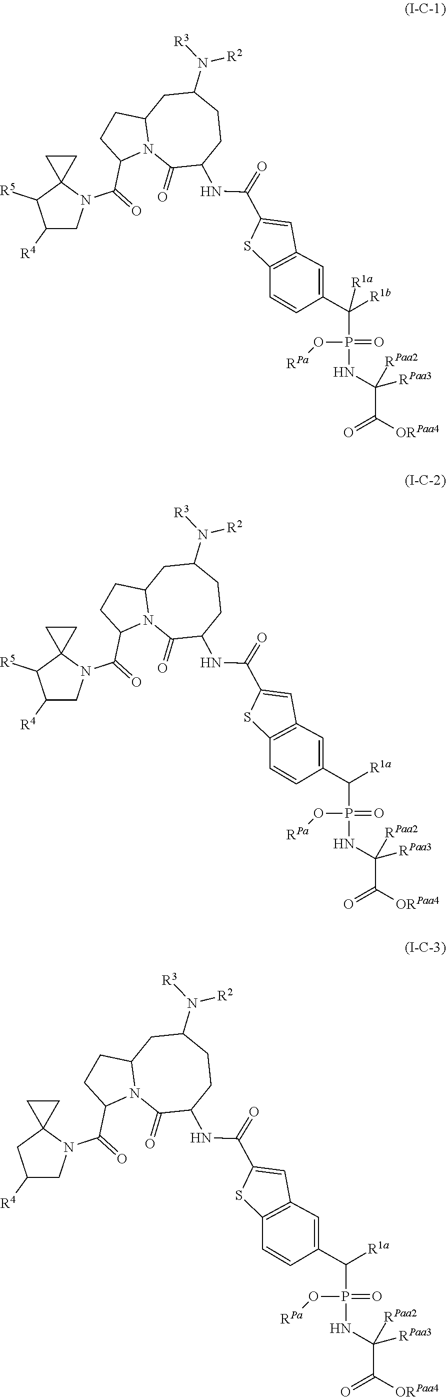

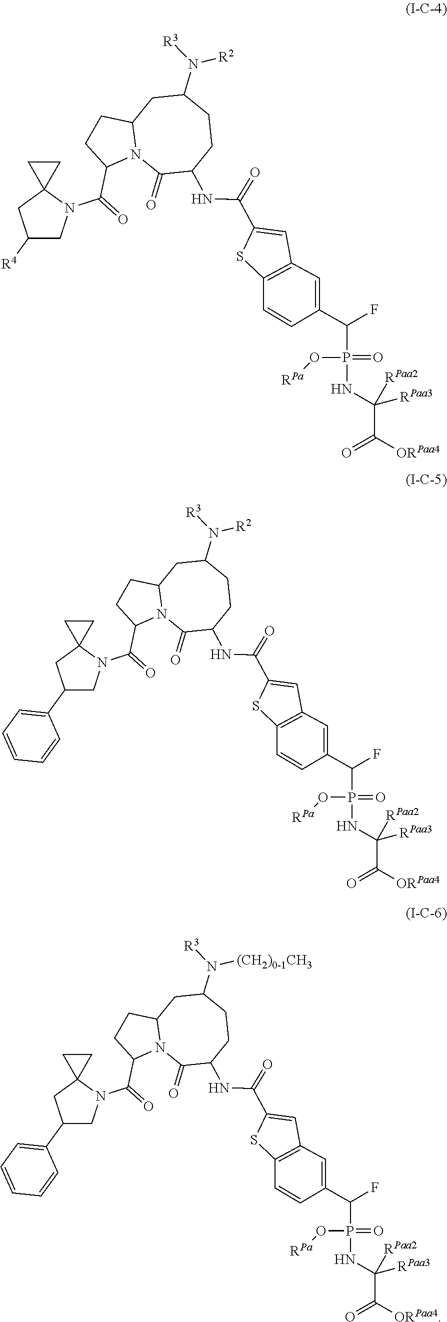

wherein R^{Pa} is (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy; R^{Paa2} is H or (C₁-C₆)alkyl; R^{Paa3} is H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3}, together with the carbon atom to which they are attached form (C₃-C₆)cycloalkyl; and R^{Paa4} is (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.

In some embodiments, the compound of Formula (I), or a pharmaceutically acceptable salt thereof, is a compound of Formula (I-D-1), (I-D-2), (I-D-3), (I-D-4), (I-D-5), or (I-D-6), or a pharmaceutically acceptable salt thereof:

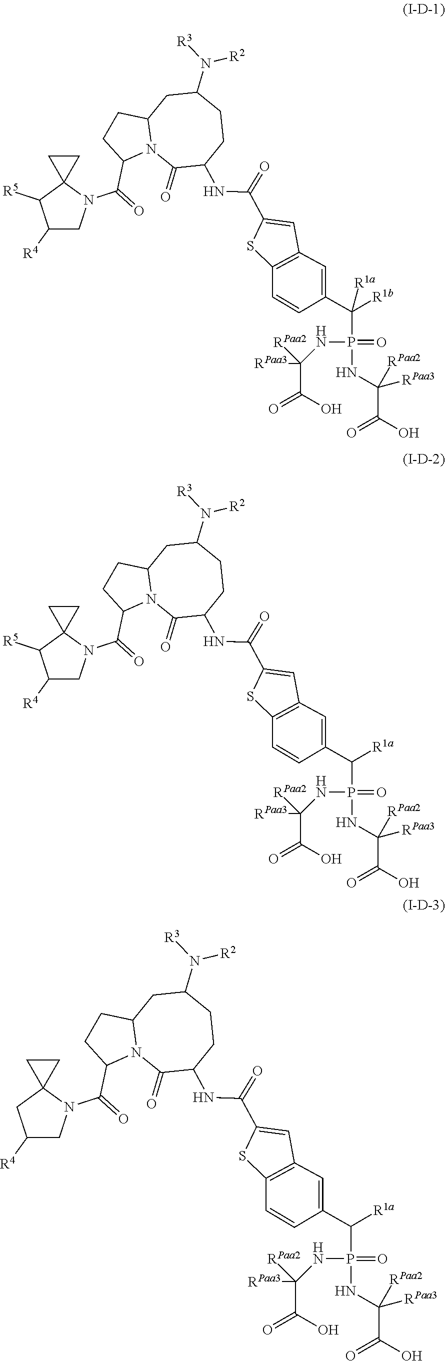

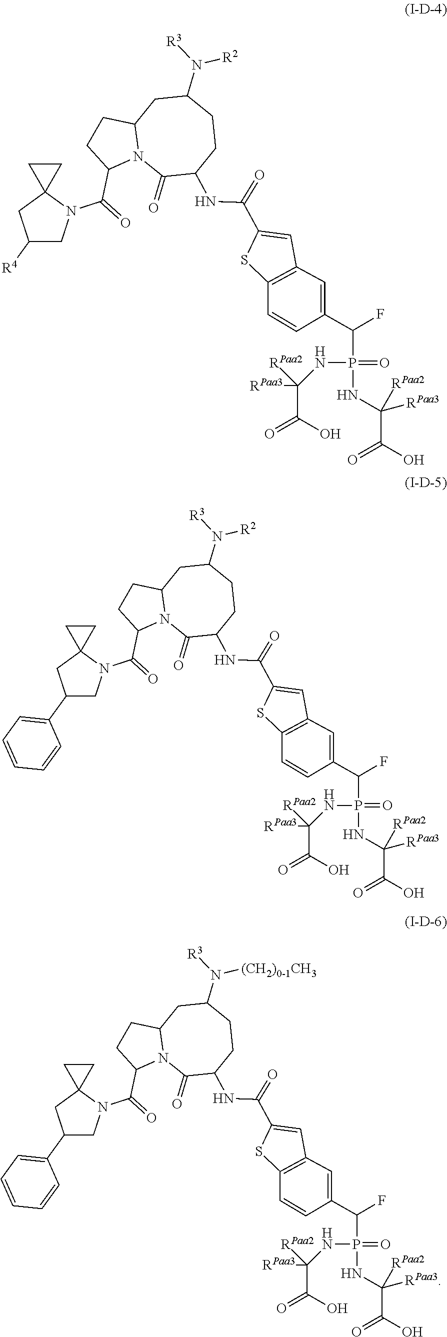

wherein each R^{Paa2} is independently H or (C₁-C₆)alkyl; and each R^{Paa3} is independently H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3} together with the carbon atom to which they are attached form (C₃-C₆)cycloalkyl.

In some embodiments, the compound of Formula (I), or a pharmaceutically acceptable salt thereof, is a compound of Formula (I-E-1), (I-E-2), (I-E-3), (I-E-4), (I-E-5), or (I-E6, or a pharmaceutically acceptable salt thereof:

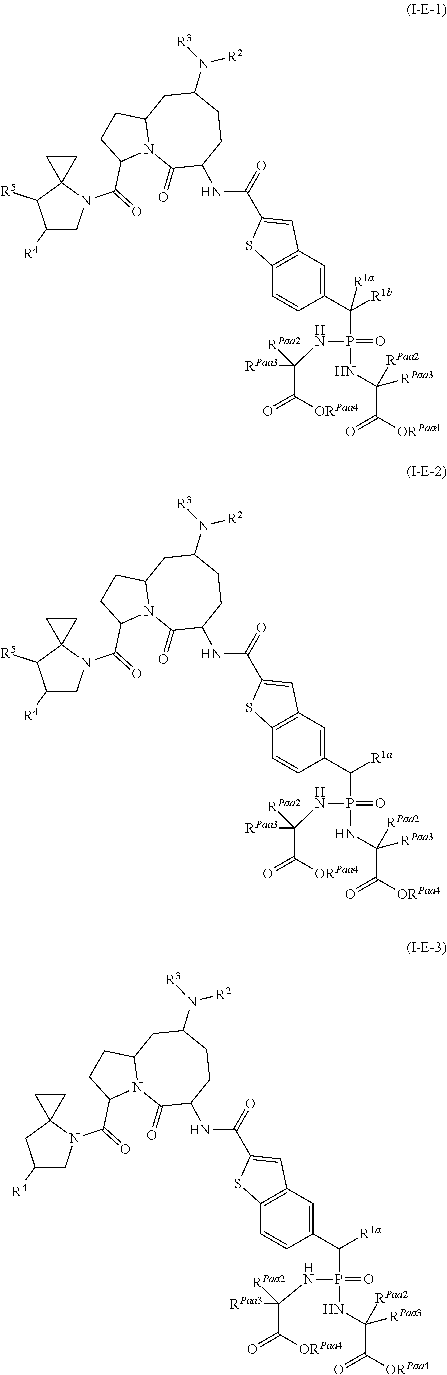

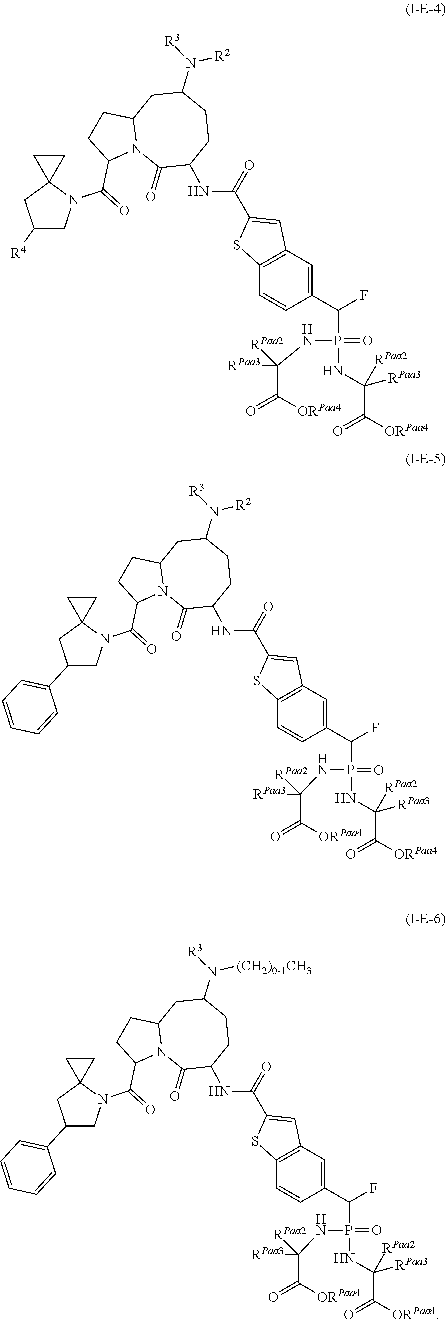

wherein R^{Paa2} is H or (C₁-C₆)alkyl; R^{Paa3} is H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3}, together with the carbon atom to which they are attached form (C₃-C₆)cycloalkyl; and R^{Paa4} is (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen. In some embodiments, the compound of Formula (I), or a pharmaceutically acceptable salt thereof, is a compound of Formula (I-F-1), (I-F-2), (I-F-3), (J-F-4), (I-F-5), or (I-F-6), or a pharmaceutically acceptable salt thereof:

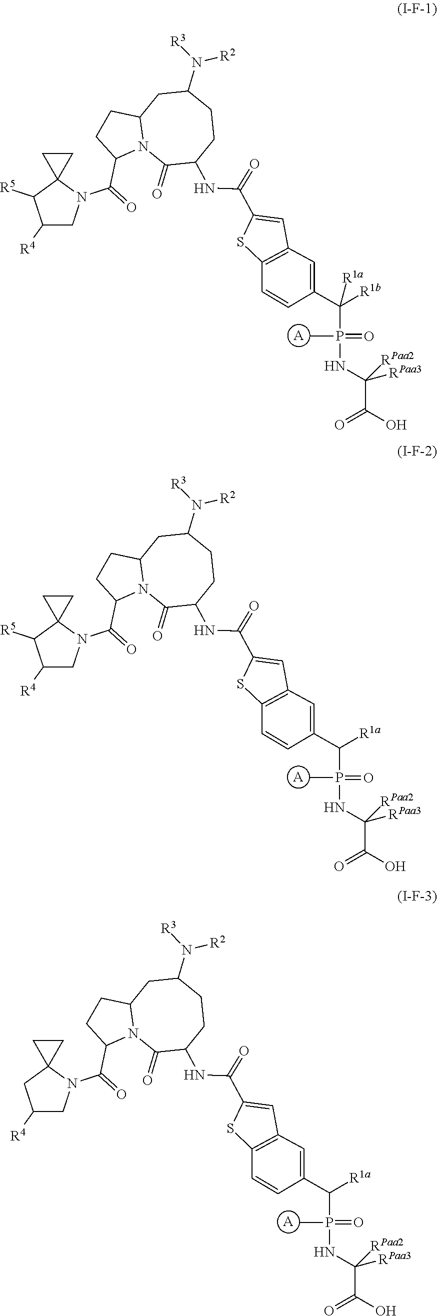

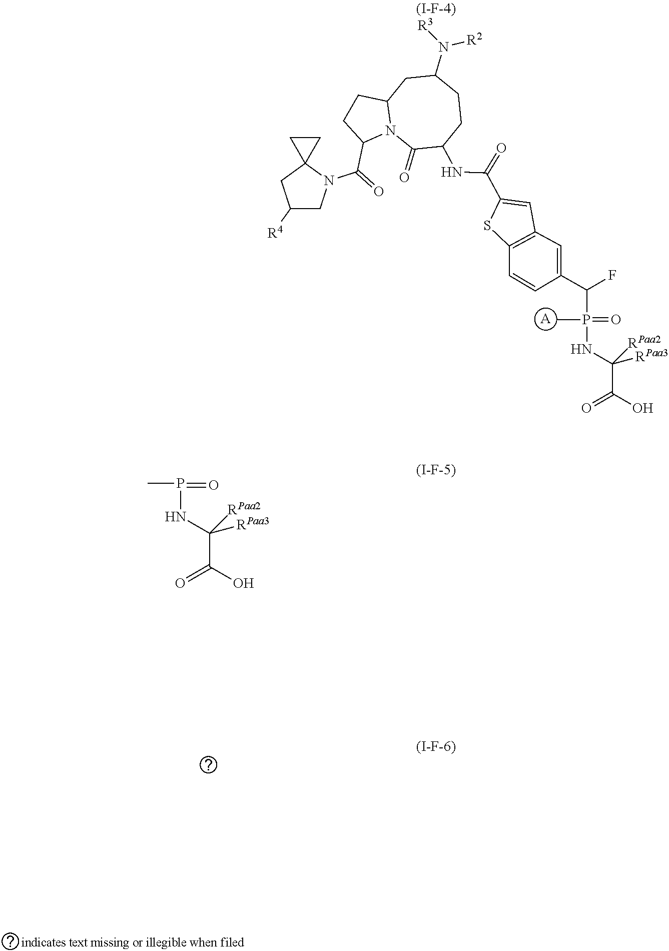

wherein R^{Paa2} is H or (C₁-C₆)alkyl; R^{Paa3} is H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3}, together with the carbon atom to which they are attached form (C₃-C₆)cycloalkyl; and Ring A is

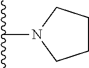 [SMILES: *N1CCCC1]

substituted with 0, 1, 2, or 3 substituents each independently selected from the group consisting of halo, oxo, COOH, and —C(O)OR^{PE}; and each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.

In some embodiments, the compound of Formula (I), or a pharmaceutically acceptable salt thereof, is a compound of Formula (I-G-1), (I-G-2), (I-G-3), (I-G-4), (I-G-5), or (I-G-6), or a pharmaceutically acceptable salt thereof:

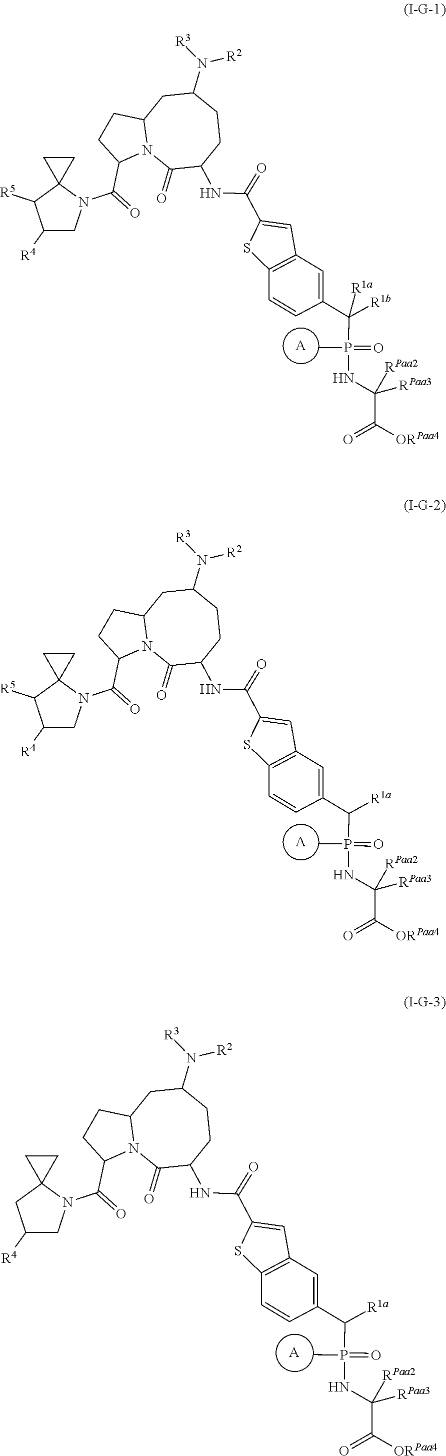

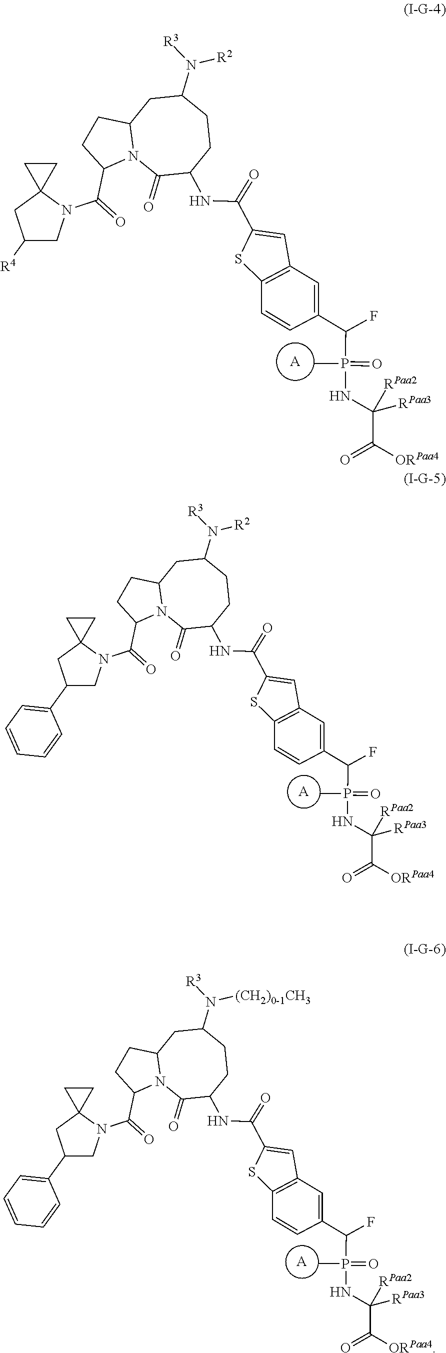

wherein R^{Paa2} is H or (C₁-C₆)alkyl; R^{Paa3} is H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3}, together with the carbon atom to which they are attached form (C₃-C₆)cycloalkyl; R^{Paa4} is (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen; and Ring A is

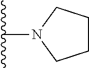 [SMILES: *N1CCCC1]

substituted, with 0, 1, 2, or 3 substituents each independently selected from the group consisting of halo, oxo, COOH, and —C(O)OR^{PE}; and each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.

In some embodiments, the compound of Formula (I), or a pharmaceutically acceptable salt thereof, is a compound of Formula (I-H-1), (I-H-2), (I-H-3), (I-H-4), (I-H-5), or (I-H-6), or a pharmaceutically acceptable salt thereof:

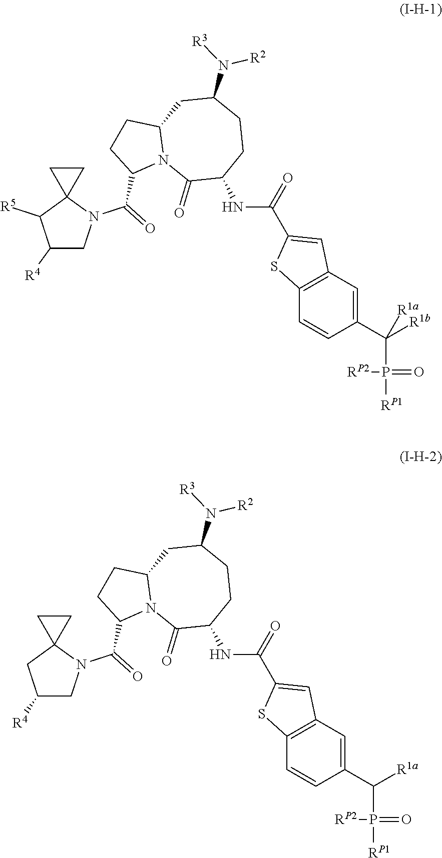

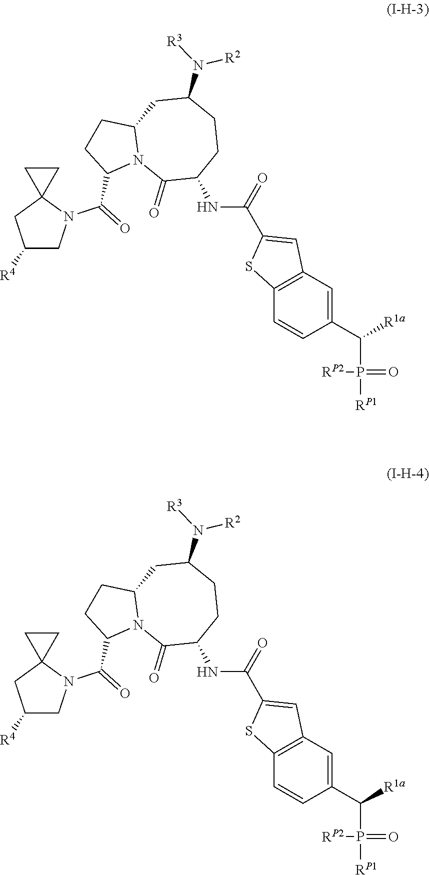

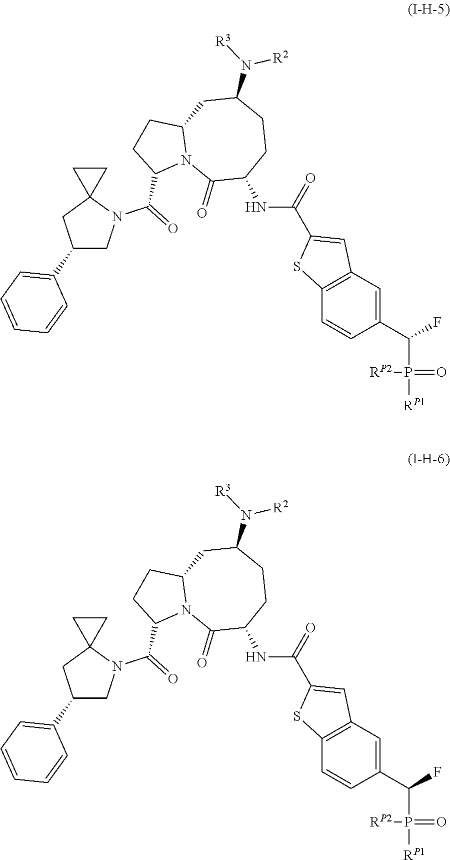

wherein R^{P1} and R^{P2} are each independently —OH, —OR^{Pa}, —NR^{Paa1} CR^{Paa2}R^{Paa3}C(O)OH, NR^{Paa1}CR^{Paa2}R^{Paa3}(O)OR^{Paa4}, or Ring A; Ring A is

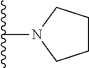 [SMILES: *N1CCCC1]

substituted with 0, 1, 2, or 3 substituents each independently selected from the group consisting of halo, oxo, COOH, and —C(O)OR^{PE}; R^{Pa} is independently (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy; each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅—C₈)spirocycloalkyl, or (C₃-C₇)cycloalkyl of —(C₁-C₆)alkylene-(C₃-C₇)cycloalkyl is each substituted with 0, 1, 2, or 3 independently selected halogen; each R^{Paa1} is independently H or (C₁-C₆)alkyl; each R^{Paa2} is independently H or (C₁-C₆)alkyl; each R^{Paa3} is independently H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3}, together with the carbon atom to which they are attached form (C₃-C₆)cycloalkyl; and each R^{Paa4} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.

In some embodiments, R^{1a} and R^{1b} are each independently H or halogen. In some embodiments, R² is H, (C₁-C₆)alkyl, or (C₃-C₆)cycloalkyl. In some embodiments, R³ is (C₁-C₆)alkyl, halo(C₁-C₆)alkyl, (C₃-C₆)cycloalkyl, 3- to 7-membered heterocyclyl, —(C₁-C₆)alkylene-(C₃-C₆)cycloalkyl, or —(C₁-C₆)alkylene-(3- to 7-membered heterocyclyl), wherein (C₁-C₆)alkyl, (C₃-C₆)cycloalkyl, (C₃-C₆)cycloalkyl of —(C₁-C₆)alkylene-(C₃-C₆)cycloalkyl, or (3- to 7-membered heterocyclyl) of —(C₁-C₆)alkylene-(3- to 7-membered heterocyclyl) is each substituted with 0, 1, 2, 3, 4, or 5 substituents each independently selected from the group consisting of (C₁-C₆)alkyl, (C₁-C₆)alkoxy, cyano, halogen, halo(C₁-C₆)alkyl, and halo(C₁-C₆)alkoxy. In some embodiments, R² and R³ together with the nitrogen atom to which they are attached form 5- to 7-membered heterocyclyl substituted with 0, 1, 2, or 3 substituents each independently selected from the group consisting of (C₁-C₆)alkyl, (C₁-C₆)alkoxy, cyano, halogen, halo(C₁-C₆)alkyl, and halo(C₁-C₆)alkoxy. In some embodiments, R⁴ is H, (C₆-C₁₀)aryl, or 5- to 10-membered heteroaryl, wherein (C₆-C₁₀)aryl or 5- to 10-membered heteroaryl is each substituted with 0, 1, 2, or 3 substituents each independently selected from the group consisting of halogen, cyano, (C₁-C₆)alkyl, (C₁-C₆)alkoxy, halo(C₁-C₆)alkyl, halo(C₁-C₆)alkoxy, and —NR^{4a}R^{4b}, wherein: each R^{4a} and R^{4b} is independently H or (C₁-C₆)alkyl. In some embodiments, R⁵ is H, cyano, or (C₁-C₆)alkoxy. In some embodiments, R^{P} is phosphonic acid, phosphonate, phosphonamidate, or phosphondiamidate.

In some embodiments, R^{1a} is F. In some embodiments, R^{1b} is H. In some embodiments, R^{1a} is F and R^{1b} is H. In some embodiments, R^{1a} and R^{1b} are each F.

In some embodiments, R² is methyl or ethyl.

In some embodiments, R³ is (C₁-C₆)alkyl, halo(C₁-C₆)alkyl, (C₃-C₆)cycloalkyl, 3- to 7-membered heterocyclyl, or —(C₁-C₆)alkylene-(3- to 7-membered heterocyclyl). In some embodiments, R³ is halo(C₁-C₆)alkyl. In some embodiments, R³ is (C₃-C₆)cycloalkyl substituted with 0, 1, 2, or 3 substituents each independently selected from the group consisting of halogen, cyano, halo(C₁-C₆)alkyl, and halo(C₁-C₆)alkoxy. In some embodiments, R³ is 3- to 7-membered heterocyclyl. In some embodiments, R³ is —(C₁-C₆)alkylene-[3- to 7-membered heterocyclyl substituted with 0 or 1 (C₁-C₆)alkyl]. In some embodiments, R³ is (C₁-C₆)alkyl substituted with 0, 1, 2, 3, 4, or 5 substituents each independently selected from the group consisting of halo, (C₁-C₆)alkoxy, and halo(C₁-C₆)alkoxy. In some embodiments, R³ is

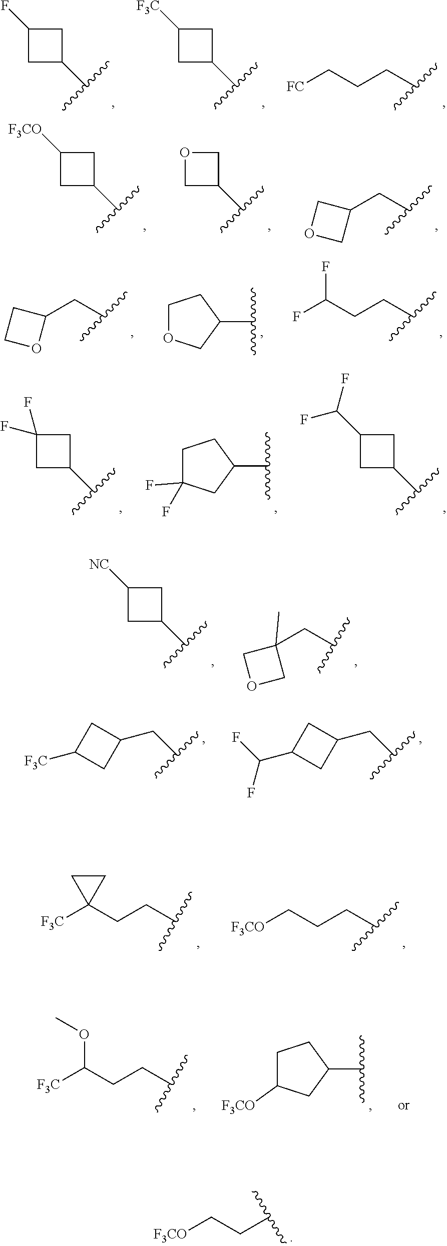

In some embodiments,

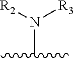

### of Formula (I) is

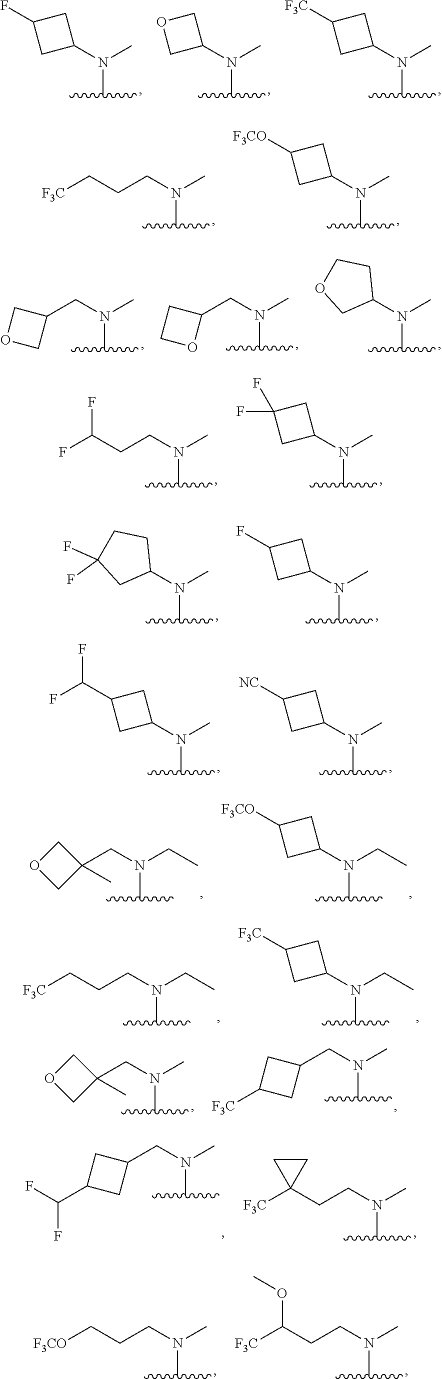

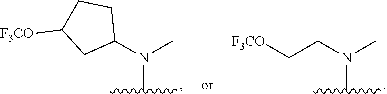

In some embodiments, R² and R³ together with the nitrogen atom to which they are attached form 6-membered heterocyclyl.

In some embodiments,

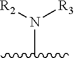

### of Formula (I) is

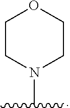

In some embodiments, R⁴ is H. In some embodiments, R⁴ is (C₆-C₁₀)aryl or 5- to 10-membered heteroaryl. In some embodiments, R⁴ is phenyl substituted with 0, 1, 2, or 3 halogen.

In some embodiments, R⁴ is

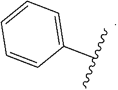 [SMILES: *c1ccccc1]

In some embodiments, R⁴ is

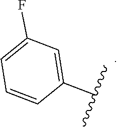 [SMILES: *c1cccc(F)c1]

In some embodiments, R⁴ is pyridinyl substituted with 0 or 1 —NR^{4a}R^{4b}. In some embodiments, R⁴ is

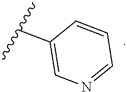 [SMILES: *c1cccnc1]

In some embodiments, R⁴ is

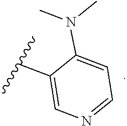 [SMILES: *c1cnccc1N(C)C]

In some embodiments, R⁵ is H. In some embodiments, R⁵ is cyano. In some embodiments, R⁵ is methoxy.

In some embodiments, R^{P} is

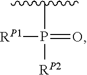

wherein R^{P1} and R^{P2} are each independently —OH, —OR^{Pa}, N-linked amino acid, N-linked amino acid ester, or Ring A. In some embodiments, Ring A is

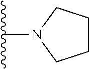 [SMILES: *N1CCCC1]

substituted with 0, 1, 2, or 3 substituents each independently selected from the group consisting of halo, oxo, COOH, and —C(O)OR^{PE}. In some embodiments, R^{Pa} is independently (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy. In some embodiments, each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.

In some embodiments, R^{P} is

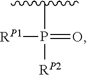

wherein R^{P1} and R^{P2} are each independently —OH, —OR^{Pa}, —NR^{Paa1}CR^{Paa2}R^{Paa3}C(O)OH, —NR^{Paa1}CR^{Paa2}R^{Paa3}C(O)OR^{Paa4}, or Ring A. In some embodiments, Ring A is

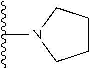 [SMILES: *N1CCCC1]

substituted with 0, 1, 2, or 3 substituents each independently selected from the group consisting of halo, oxo, COOH, and —C(O)OR^{PE}. In some embodiments, R^{Pa} is independently (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy. In some embodiments, each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen. In some embodiments, each R^{Paa1} is independently H or (C₁—C₆)alkyl. In some embodiments, each R^{Paa2} is independently H or (C₁-C₆)alkyl, and each R^{Paa3} is independently H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3}, together with the carbon atom to which they are attached form (C₃-C₆)cycloalkyl. In some embodiments, each R^{Paa4} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.

In some embodiments, R^{P} is

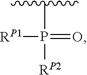

wherein R^{P1} and R^{P2} are each —OH.

In some embodiments, R^{P} is

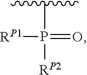

wherein R^{P1} is —OH and R^{P2} is N-linked amino acid. In some embodiments, R^{P} is

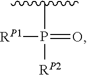

wherein R^{P1} is —OH and R^{P2} is N-linked amino acid ester.

In some embodiments, R^{P} is

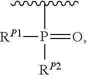

wherein R^{P1} is —OR^{Pa} and R^{P2} is N-linked amino acid ester. In some embodiments, R^{Pa} is (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy.

In some embodiments, R^{P} is

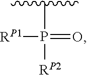

wherein R^{P1} and R^{P2} are each independently N-linked amino acid.

In some embodiments, R^{P} is

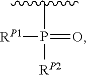

wherein R^{P1} and R^{P2} are each independently N-linked amino acid ester.

In some embodiments, R^{P} is

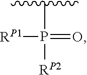

wherein R^{P1} is N-linked amino acid and R^{P2} is Ring A, wherein Ring A is

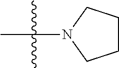 [SMILES: *N1CCCC1]

substituted with 0, 1, 2, or 3 substituents each independently selected from the group consisting of halo, oxo, and —COOH.

In some embodiments, R^{P} is

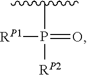

wherein R^{P1} is N-linked amino acid ester and R^{P2} is Ring A, wherein Ring A is

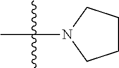 [SMILES: *N1CCCC1]

substituted with 0, 1, 2, or 3 substituents each independently selected from the group consisting of halo, oxo, and ester.

In some embodiments, R^{Pa} is phenyl. In some embodiments R^{Pa} is ethyl. In some embodiments, R^{Pa} is —CH₂CF₃. In some embodiments, R^{Pa} is

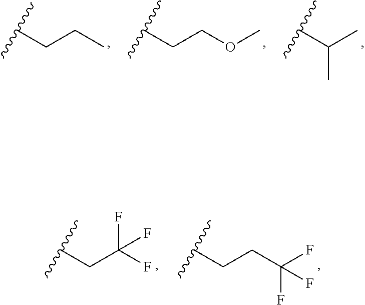

cyclopropyl, cyclopentyl, or cyclohexyl. In some embodiments, R^{Pa} is

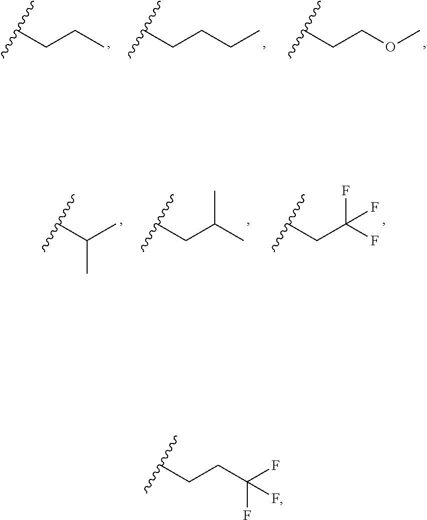

cyclopropyl, cyclopentyl, or cyclohexyl.

In some embodiments, R^{P2} is

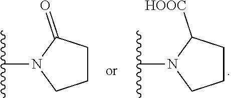 [SMILES: *N1CCCC1=O ; *N1CCCC1C(=O)O]

In some embodiments, R^{P2} is Ring A, wherein Ring A is

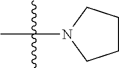 [SMILES: *N1CCCC1]

substituted with 0, 1, 2, or 3 substituents each independently selected from the group consisting of halo, oxo, and —C(O)OR^{PE}. In some embodiments, each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₅)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₅)spirocycloalkyl, or (C₃-C₇)cycloalkyl of —(C₁-C₆)alkylene-(C₃-C₇)cycloalkyl is each substituted with 0, 1, 2, or 3 independently selected halogen. In some embodiments, each R^{PE} is independently ethyl,

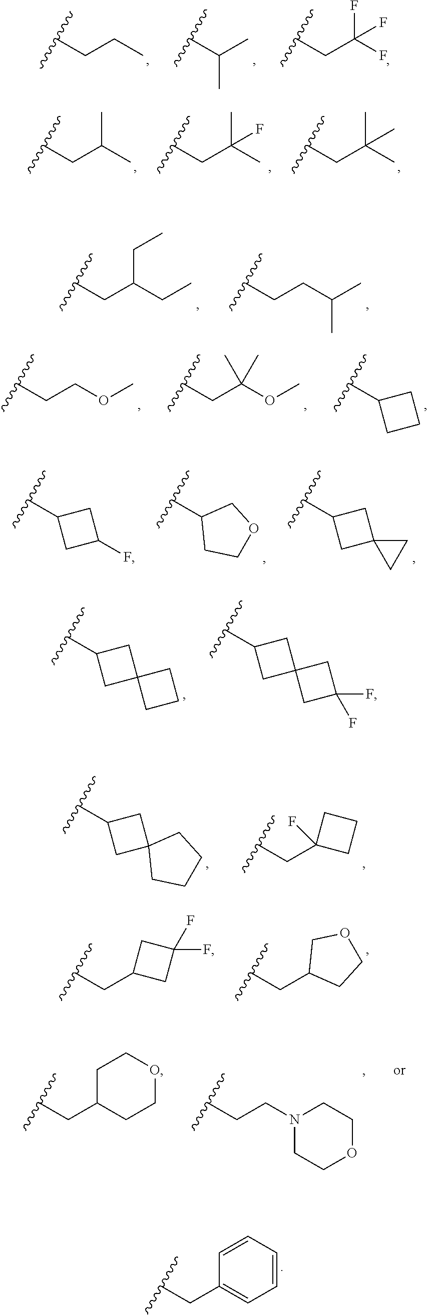

In some embodiments, each R^{PE} is

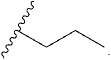 [SMILES: *CCC]

In some embodiments, R^{P2} is

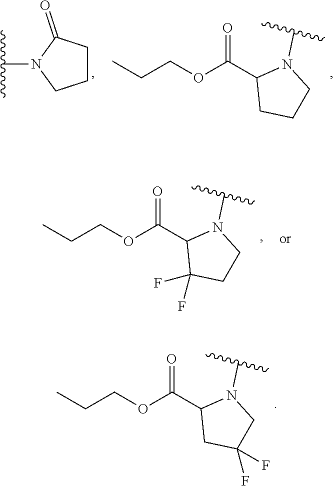

In some embodiments, each N-linked amino acid is independently N-linked α-amino acid. In some embodiments, each N-linked amino acid ester is independently N-linked α-amino acid ester.

In some embodiments, each N-linked amino acid is independently —NR^{Paa1}CR^{Paa2}R^{Paa3}C(O)OH. In some embodiments, each N-linked amino acid ester is independently —NR^{Paa1}CR^{Paa2}R^{Paa3}C(O)OR^{Paa4}.

In some embodiments, each R^{Paa1} is independently H or (C₁-C₆)alkyl. In some embodiments, each R^{Paa2} is independently H or (C₁-C₆)alkyl, and each R^{Paa3} is independently H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3}, together with the carbon atom to which they are attached form (C₃-C₆)cycloalkyl.

In some embodiments, R^{Paa1} is H. In some embodiments, R^{Paa1} is methyl.

In some embodiments, R^{Paa2} is H. In some embodiments, R^{Paa2} is methyl.

In some embodiments, R^{Paa3} is methyl. In some embodiments, R^{Paa3} is ethyl,

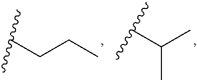

or —CH₂OCH₃. In some embodiments, R^{Paa3} is

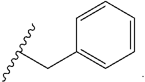 [SMILES: *Cc1ccccc1]

In some embodiments, R^{Paa2} and R^{Paa3}, together with the carbon atom to which they are attached form cyclopropyl.

In some embodiments, each R^{Paa4} is independently (C₁-C₆)alkyl, halo(C₁-C₆)alkyl, —(C₁-C₆)alkylene-(C₁-C₆)alkoxy, (C₃-C₆)cycloalkyl, (C₅-C₅)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₅)spirocycloalkyl, or (C₃-C₇)cycloalkyl of —(C₁-C₆)alkylene-(C₃-C₇)cycloalkyl is each substituted with 0, 1, 2, or 3 independently selected halogen. In some embodiments, each R^{Paa4} is independently ethyl,

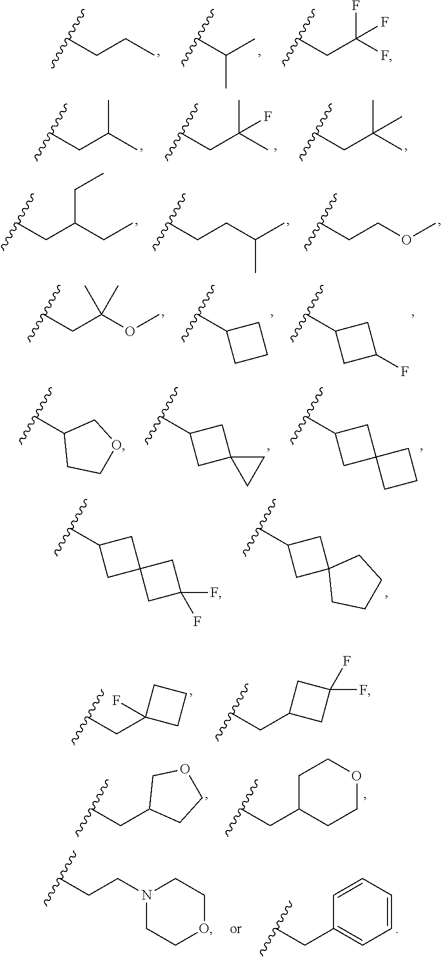

In some embodiments, R^{Paa4} is

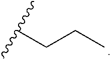 [SMILES: *CCC]

In some embodiments, R^{Paa4} is ethyl,

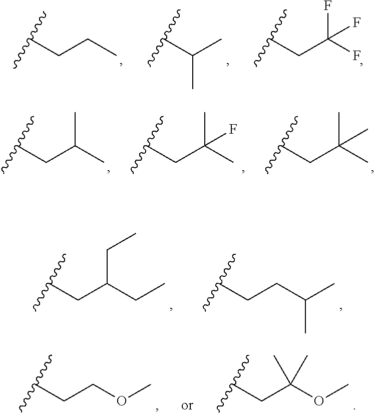

In some embodiments, R^{Paa4} is

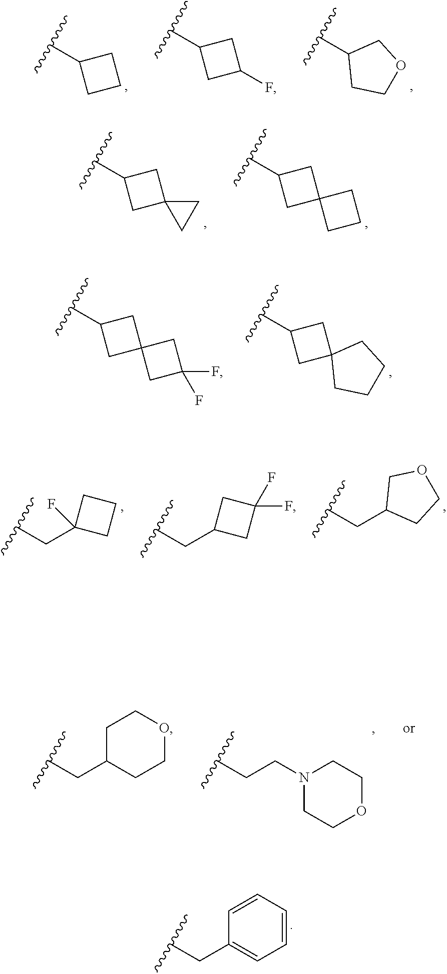

In some embodiments, provided herein are compounds of Table 1A, Table 1B, Table 1C, and pharmaceutically acceptable salts thereof. See Example 1.

In one aspect, provided herein is a parent drug. In some embodiments, a prodrug can be metabolized to produce the parent drug. In some embodiments, the prodrug can be metabolized to produce an intermediate metabolite. In some embodiments, the intermediate metabolite can be further metabolized to produce the parent drug. In some embodiments, the prodrug can be metabolized by enzymes such as carboxyesterase and/or phosphoramidase to produce the parent drug. In some embodiments, the prodrug is a ProTide prodrug analog (see, e.g., Mehellou, et al., J. Med. Chem. 61(6) 2211-2226 (2018). In some embodiments, the prodrug has an R^{P} selected from those present in molecules described herein in the representative procedures for synthesis of phosphoryl groups of Example 1.

In some embodiments, the parent drug is the compound of Formula (I-A-1), (I-A-2), (I-A-3), (I-A-4), (I-A-5), or (I-A-6), or a pharmaceutically acceptable salt thereof. In some embodiments, the parent drug is a compound of Formula (I), wherein R^{P} is

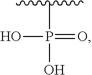 [SMILES: *P(=O)(O)O]

or a pharmaceutically acceptable salt thereof. In some embodiments, the parent drug is a compound selected from the group consisting of the compounds of Table 1A, or a pharmaceutically acceptable salt thereof.

In some embodiments, the compound, or a pharmaceutically acceptable salt thereof, is a parent drug selected from the group consisting of:

(fluoro(2-((9-((3-fluorocyclobutyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((9-(methyl(4,4,4-trifluorobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
((2-((3-(7-cyano-6-phenyl-4-azaspiro[2.4]heptane-4-carbonyl)-9-morpholino-5-
oxodecahydropyrrolo[1,2-a]azocin-6-yl)carbamoyl)benzo[b]thiophen-5-
yl)fluoromethyl)phosphonic acid;
(fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)phosphonic acid;
((2-((3-(6-(4-(dimethylamino)pyridin-3-yl)-4-azaspiro[2.4]heptane-4-carbonyl)-9-morpholino-5-
oxodecahydropyrrolo[1,2-a]azocin-6-yl)carbamoyl)benzo[b]thiophen-5-
yl)fluoromethyl)phosphonic acid;
(fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)phosphonic acid;
((2-((3-(7-cyano-6-(pyridin-3-yl)-4-azaspiro[2.4]heptane-4-carbonyl)-9-morpholino-5-
oxodecahydropyrrolo[1,2-a]azocin-6-yl)carbamoyl)benzo[b]thiophen-5-
yl)fluoromethyl)phosphonic acid;
((2-((3-(6-(4-(dimethylamino)pyridin-3-yl)-4-azaspiro[2.4]heptane-4-carbonyl)-9-morpholino-5-
oxodecahydropyrrolo[1,2-a]azocin-6-yl)carbamoyl)benzo[b]thiophen-5-
yl)fluoromethyl)phosphonic acid;
(fluoro(2-((9-(methyl(oxetan-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((9-(methyl(oxetan-3-ylmethyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((9-(methyl((oxetan-2-yl)methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((9-(methyl(oxetan-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((9-(methyl((oxetan-2-yl)methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((9-(methyl(tetrahydrofuran-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((9-(methyl(tetrahydrofuran-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
((2-((9-((3,3-difluoropropyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
(fluoro(2-((9-((3-fluorocyclobutyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((9-((3-fluorocyclobutyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
2,2,2-trifluoroacetic acid compound with ((2-((9-((3,3-difluorocyclobutyl)(methyl)amino)-5-
oxo-3-(6-phenyl-4-azaspiro[2.4]heptane-4-carbonyl)decahydropyrrolo[1,2-a]azocin-6-
yl)carbamoyl)benzo[b]thiophen-5-yl)fluoromethyl)phosphonic acid;
(fluoro(2-((9-(methyl((oxetan-2-yl)methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
((2-((9-((3,3-difluorocyclobutyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
(fluoro(2-((9-(methyl(tetrahydrofuran-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((3-(6-(3-fluorophenyl)-4-azaspiro[2.4]heptane-4-carbonyl)-9-(methyl(oxetan-3-
yl)amino)-5-oxodecahydropyrrolo[1,2-a]azocin-6-yl)carbamoyl)benzo[b]thiophen-5-
yl)methyl)phosphonic acid;
(fluoro(2-((9-(methyl(tetrahydrofuran-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
((2-((9-((3,3-difluoropropyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
(fluoro(2-((9-((3-fluorocyclobutyl)(methyl)amino)-5-oxo-3-(6-(pyridin-3-yl)-4-
azaspiro[2.4]heptane-4-carbonyl)decahydropyrrolo[1,2-a]azocin-6-
yl)carbamoyl)benzo[b]thiophen-5-yl)methyl)phosphonic acid.;
(fluoro(2-((9-(methyl((oxetan-2-yl)methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
((2-((9-((3,3-difluorocyclopentyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
((2-((9-(ethyl(3-fluorocyclobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
((2-((9-(ethyl(3-fluorocyclobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
((2-((9-((3-(difluoromethyl)cyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)phosphonic acid;
((2-((9-((3,3-difluorocyclopentyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
((2-((9-((3-cyanocyclobutyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
(fluoro(2-((9-(methyl(4,4,4-trifluorobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
(fluoro(2-((9-((3-fluorocyclobutyl)(methyl)amino)-5-oxo-3-(4-azaspiro[2.4]heptane-4-
carbonyl)decahydropyrrolo[1,2-a]azocin-6-yl)carbamoyl)benzo[b]thiophen-5-
yl)methyl)phosphonic acid;
((2-((9-((3-cyanocyclobutyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
2,2,2-trifluoroacetic acid compound with ((2-((9-(ethyl((3-methyloxetan-3-yl)methyl)amino)-5-
oxo-3-(6-phenyl-4-azaspiro[2.4]heptane-4-carbonyl)decahydropyrrolo[1,2-a]azocin-6-
yl)carbamoyl)benzo[b]thiophen-5-yl)fluoromethyl)phosphonic acid;
((2-((9-(ethyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)phosphonic acid;
((2-((9-(ethyl(4,4,4-trifluorobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
((2-((9-(ethyl(4,4,4-trifluorobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
((2-((9-(ethyl(3-(trifluoromethyl)cyclobutyl)amino)-5-oxo-3-(6-phenyl-4-azaspiro[2.4]heptane-
4-carbonyl)decahydropyrrolo[1,2-a]azocin-6-yl)carbamoyl)benzo[b]thiophen-5-
yl)fluoromethyl)phosphonic acid;
((2-((9-(ethyl(3-(trifluoromethyl)cyclobutyl)amino)-5-oxo-3-(6-phenyl-4-azaspiro[2.4]heptane-
4-carbonyl)decahydropyrrolo[1,2-a]azocin-6-yl)carbamoyl)benzo[b]thiophen-5-
yl)fluoromethyl)phosphonic acid;
((2-((9-(ethyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)phosphonic acid;
(fluoro(2-((9-(methyl((3-(trifluoromethyl)cyclobutyl)methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((9-(methyl((3-(trifluoromethyl)cyclobutyl)methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((9-(methyl(2-(1-(trifluoromethyl)cyclopropyl)ethyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((9-(methyl(3-(trifluoromethoxy)cyclobutyl)amino)-5-oxo-3-(4-azaspiro[2.4]heptane-
4-carbonyl)decahydropyrrolo[1,2-a]azocin-6-yl)carbamoyl)benzo[b]thiophen-5-
yl)methyl)phosphonic acid;
(fluoro(2-((9-(methyl(3-(trifluoromethoxy)propyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((9-(methyl(4,4,4-trifluoro-3-methoxybutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((9-morpholino-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)phosphonic acid;
((2-((9-((3,3-difluorocyclopentyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)phosphonic acid;
(fluoro(2-((9-(methyl((3-methyloxetan-3-yl)methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((9-(methyl(3-(trifluoromethoxy)cyclopentyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
(difluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((9-(methyl(3-(trifluoromethoxy)cyclobutyl)amino)-5-oxo-3-(6-(pyridin-3-yl)-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((9-(methyl(3-(trifluoromethoxy)cyclobutyl)amino)-5-oxo-3-(6-(pyridin-3-yl)-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((3-(7-methoxy-6-phenyl-4-azaspiro[2.4]heptane-4-carbonyl)-9-(methyl(3-
(trifluoromethoxy)cyclobutyl)amino)-5-oxodecahydropyrrolo[1,2-a]azocin-6-
yl)carbamoyl)benzo[b]thiophen-5-yl)methyl)phosphonic acid;
(fluoro(2-((3-(7-methoxy-6-phenyl-4-azaspiro[2.4]heptane-4-carbonyl)-9-(methyl(3-
(trifluoromethoxy)cyclobutyl)amino)-5-oxodecahydropyrrolo[1,2-a]azocin-6-
yl)carbamoyl)benzo[b]thiophen-5-yl)methyl)phosphonic acid;
(fluoro(2-((9-(methyl(3,3,3-trifluoro-2-methoxypropyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid;
(fluoro(2-((9-(methyl(2-(trifluoromethoxy)ethyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid; and
(fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphonic acid,

and pharmaceutically acceptable salts thereof.

In some embodiments, the prodrug is a compound of Formula (I), wherein R^{P} is

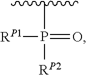

wherein R^{P1} and R^{P2} are each independently —OR^{Pa}, or a pharmaceutically acceptable salt thereof.

In some embodiments, the prodrug is the compound of Formula (I-C-1), (I-C-2), (I-C-3), (I-C-4), (I-C-5), or (I-C-6), or a pharmaceutically acceptable salt thereof. In some embodiments, the prodrug is a compound of Formula (I), wherein R^{P} is

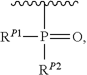

wherein R^{P1} is —OR^{Pa} and R^{Paa2} is N-linked amino acid ester, or a pharmaceutically acceptable salt thereof. In some embodiments, the intermediate metabolite of the prodrug is the compound of Formula (I-B-1), (I-B-2), (I-B-3), (I-B-4), (I-B-5), or (I-B-6), or a pharmaceutically acceptable salt thereof. In some embodiments, the intermediate metabolite of the prodrug is a compound of Formula (I), wherein R^{P} is

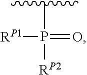

wherein R^{P1} is —OH and R^{Paa2} is N-linked amino acid, or a pharmaceutically acceptable salt thereof. In some embodiments, the intermediate metabolite is a compound of formula (I), wherein R^{P} is

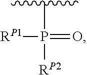

wherein R^{P1} is —OH, and R^{P2} is

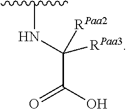

In some embodiments, the intermediate metabolite is a compound of formula (I), wherein R^{P} is

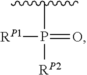

wherein R^{P1} is —OH, and R^{P2} is

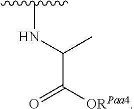

In some embodiments, the intermediate metabolite is a compound of formula (I), wherein R^{P} is

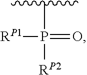

wherein R^{P1} is —OH, and R^{P2} is

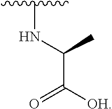 [SMILES: *N[C@@H](C)C(=O)O]

In some embodiments, the prodrug is the compound of Formula (I-E-1), (I-E-2), (I-E-3), (I-E-4), (I-E-5), or (I-E-6), or a pharmaceutically acceptable salt thereof. In some embodiments, the prodrug is a compound of Formula (I), wherein R^{P} is

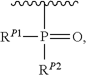

wherein R^{P1} and R^{P2} are each independently N-linked amino acid ester, or a pharmaceutically acceptable salt thereof. In some embodiments, the intermediate metabolite of the prodrug is the compound of Formula (I-D-1), (I-D-2), (I-D-3), (I-D-4), (I-D-5), or (I-D-6), or a pharmaceutically acceptable salt thereof. In some embodiments, the intermediate metabolite of the prodrug is a compound of Formula (I), wherein R^{P} is

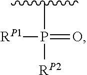

wherein R^{P1} and R^{P2} are each independently N-linked amino acid, or a pharmaceutically acceptable salt thereof.

In some embodiments, the prodrug is the compound of Formula (I-G-1), (I-G-2), (I-G-3), (I-G-4), (I-G-5), or (I-G-6), or a pharmaceutically acceptable salt thereof, wherein Ring A is substituted with 0, 1, 2, or 3 substituents each independently selected from the group consisting of halo, oxo, and ester. In some embodiments, the prodrug is a compound of Formula (I), wherein R^{P} is

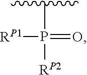

wherein R^{P1} is N-linked amino acid ester and R^{P2} is Ring A, wherein Ring A is

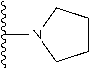 [SMILES: *N1CCCC1]

substituted with 0, 1, 2, or 3 substituents each independently selected from the group consisting of halo, oxo, and ester, or a pharmaceutically acceptable salt thereof. In some embodiments, the intermediate metabolite of the prodrug is the compound of Formula (I-F-1), (I-F-2), (I-F-3), (I-F-4), (I-F-5), or (I-F-6), or a pharmaceutically acceptable salt thereof, wherein Ring A is substituted with 0, 1, 2, or 3 substituents each independently selected from the group consisting of halo, oxo, and —COOH. In some embodiments, the intermediate metabolite of the prodrug is a compound of Formula (I), wherein R^{P} is

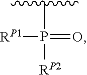

wherein R^{P1} is N-linked amino acid and R^{P2} is Ring A, wherein Ring A is

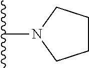 [SMILES: *N1CCCC1]

substituted with 0, 1, 2, or 3 substituents each independently selected from the group consisting of halo, oxo, and —COOH, or a pharmaceutically acceptable salt thereof.

In some embodiments, the prodrug is a compound selected from the group consisting of the compounds of Table 1C, or a pharmaceutically acceptable salt thereof. In some embodiments, the intermediate metabolite is a compound selected from the group consisting of the compounds of Table 1B, or a pharmaceutically acceptable salt thereof.

In some embodiments, the intermediate metabolite is a compound of formula (I), wherein R^{P} is

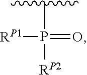

wherein R^{P1} is —OH, and R^{Paa2} is N-linked amino acid ester. In some embodiments, the intermediate metabolite is a compound of formula (I), wherein R^{P} is

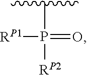

wherein R^{P1} is —OH, and R^{P2} is

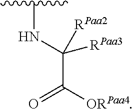

In some embodiments, the intermediate metabolite is a compound of formula (I), wherein R^{P} is

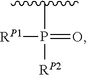

wherein R^{P1} is —OH, and R^{P2} is

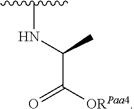

In some embodiments, the intermediate metabolite is a compound of formula (I),
wherein R^{P} is

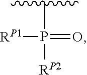

wherein R^{P1} is —OH, R^{P2} is

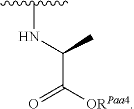

### and R^{Paa4} is

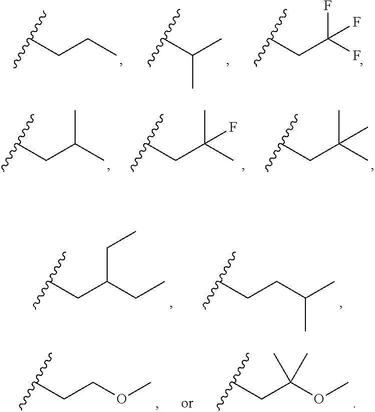

In some embodiments, the intermediate metabolite is a compound of formula (I), wherein R^{P} is

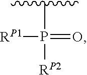

wherein R^{P1} is —OH, R^{P2} is

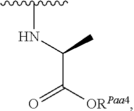

### and R^{Paa4} is

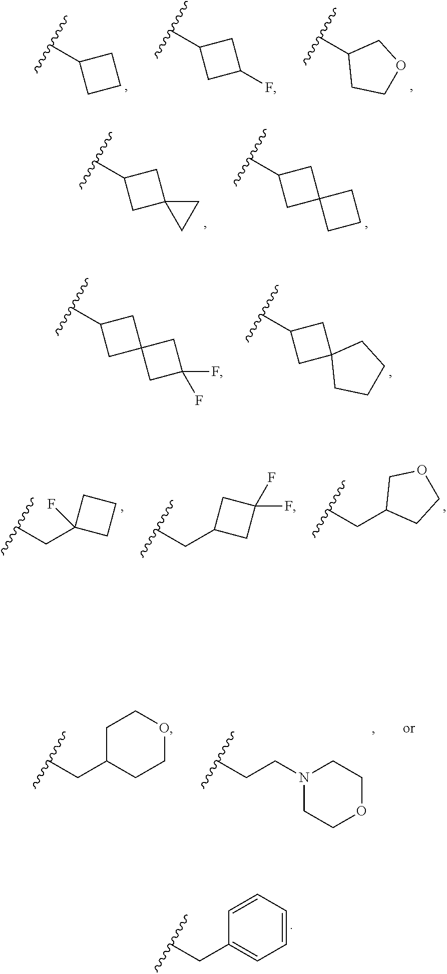

In some embodiments, the intermediate metabolite is a compound of formula (I), wherein R^{P} is

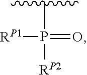

wherein R^{P1} is —OH, R^{P2} is

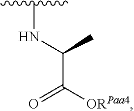

### and R^{Paa4} is

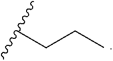 [SMILES: *CCC]

In some embodiments, the compound of Formula (I), or a pharmaceutically acceptable salt thereof, is a compound of Formula (I-J-1), (I-J-2), (I-J-3), (I-J-4), (I-J-5), or (I-J-6), or a pharmaceutically acceptable salt thereof:

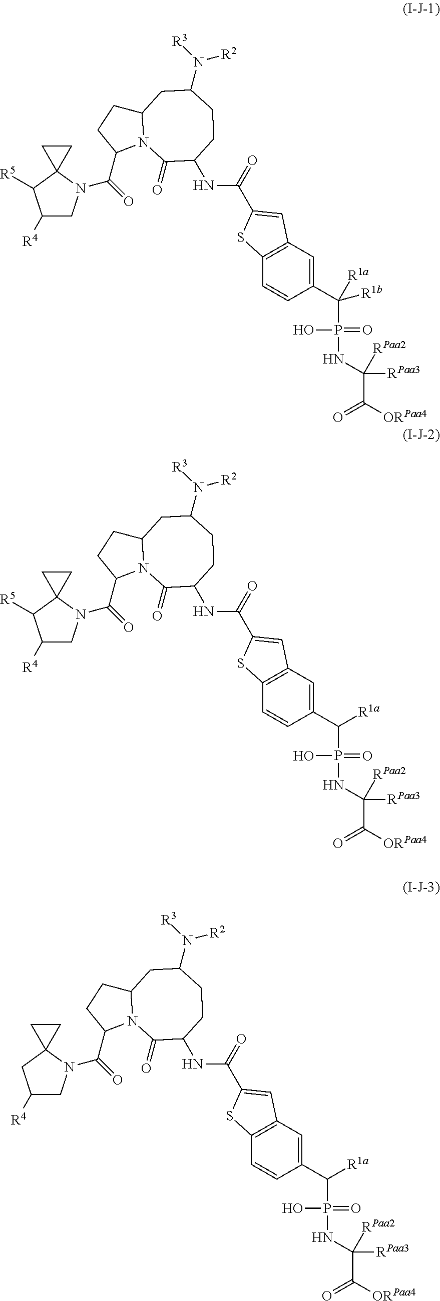

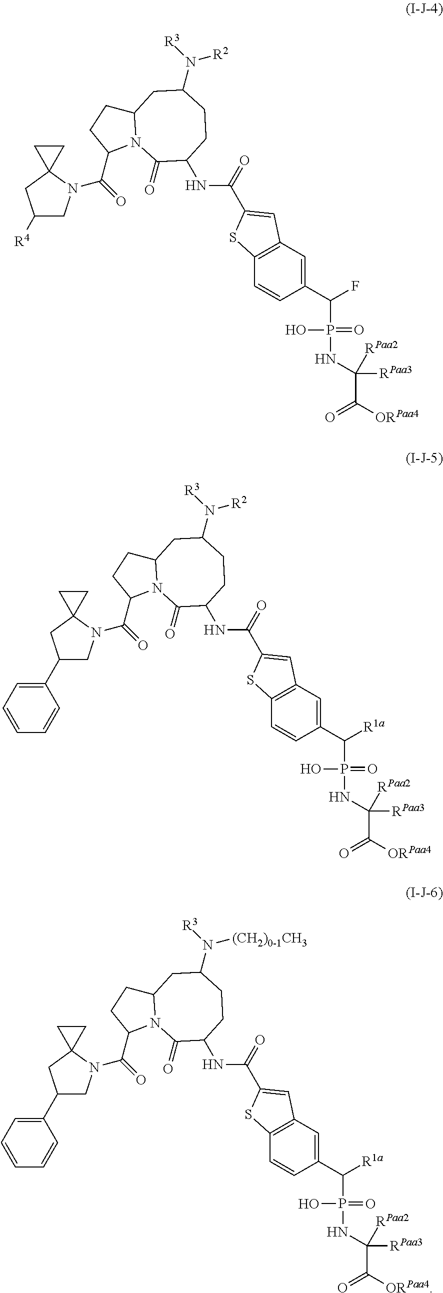

wherein R^{Paa2} is H or (C₁-C₆)alkyl; and R^{Paa3} is H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3} together with the carbon atom to which they are attached form (C₃-C₆)cycloalkyl; and R^{Paa4} is (C₁-C₆)alkyl.

In some embodiments, the compound, or a pharmaceutically acceptable salt thereof, is an intermediate metabolite selected from the group consisting of:

((fluoro(2-((9-((3-fluorocyclobutyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(hydroxy)phosphoryl)-alanine;
((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(hydroxy)phosphoryl)-alanine;
((fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(hydroxy)phosphoryl)-alanine;
((fluoro(2-((9-(methyl(4,4,4-trifluorobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
(((2-((9-((3,3-difluoropropyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)(hydroxy)phosphoryl)-alanine;
((fluoro(2-((9-((3-fluorocyclobutyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
(((2-((9-(ethyl(3-fluorocyclobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)(hydroxy)phosphoryl)-alanine;
((fluoro(2-((9-(methyl((3-methyloxetan-3-yl)methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(hydroxy)phosphoryl)-alanine;
((fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(hydroxy)phosphoryl)-alanine;
(((2-((9-((3-cyanocyclobutyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)(hydroxy)phosphoryl)-alanine;
(((2-((9-((3,3-difluorocyclopentyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)(hydroxy)phosphoryl)-alanine;
((fluoro(2-((9-((3-fluorocyclobutyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-6-
yl)methyl)(hydroxy)phosphoryl)-alanine;
((fluoro(2-((9-(methyl(4,4,4-trifluorobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
((fluoro(2-((9-((3-fluorocyclobutyl)(methyl)amino)-5-oxo-3-(4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
(((2-((9-(ethyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(hydroxy)phosphoryl)-alanine;
(((2-((9-(ethyl(4,4,4-trifluorobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)(hydroxy)phosphoryl)-alanine;
(((2-((9-((3-(difluoromethyl)cyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(hydroxy)phosphoryl)-alanine;
((fluoro(2-((9-(methyl(tetrahydrofuran-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
((fluoro(2-((9-(methyl((oxetan-2-yl)methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
((fluoro(2-((9-(methyl(oxetan-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
((fluoro(2-((9-(methyl((oxetan-2-yl)methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
((fluoro(2-((9-(methyl((oxetan-2-yl)methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
((fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
((fluoro(2-((9-((3-fluorocyclobutyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine;
(((2-((9-(ethyl(4,4,4-trifluorobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)(hydroxy)phosphoryl)-alanine;
(((2-((9-(ethyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(hydroxy)phosphoryl)-alanine; and
((fluoro(2-((9-(methyl(oxetan-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(hydroxy)phosphoryl)-alanine,

and pharmaceutically acceptable salts thereof.

In some embodiments, the compound, or a pharmaceutically acceptable salt thereof, is a prodrug selected from the group consisting of:

propyl (((2-((9-((3,3-difluoropropyl)(methyl)amino)-5-oxo-3-(6-phenyl-4-azaspiro[2.4]heptane-
4-carbonyl)decahydropyrrolo[1,2-a]azocin-6-yl)carbamoyl)benzo[b]thiophen-5-
yl)fluoromethyl)(phenoxy)phosphoryl)-alaninate;
propyl (((2-((9-((3,3-difluorocyclobutyl)(methyl)amino)-5-oxo-3-(6-phenyl-4-
azaspiro[2.4]heptane-4-carbonyl)decahydropyrrolo[1,2-a]azocin-6-yl)carbamoyl)-2,3-
dihydrobenzo[b]thiophen-5-yl)fluoromethyl)(phenoxy)phosphoryl)-alaninate;
propyl (((2-((9-((3,3-difluorocyclobutyl)(methyl)amino)-5-oxo-3-(6-phenyl-4-
azaspiro[2.4]heptane-4-carbonyl)decahydropyrrolo[1,2-a]azocin-6-yl)carbamoyl)-2,3-
dihydrobenzo[b]thiophen-5-yl)fluoromethyl)(phenoxy)phosphoryl)-alaninate;
propyl ((fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-yl)carbamoyl)-2,3-
dihydrobenzo[b]thiophen-5-yl)methyl)(phenoxy)phosphoryl)-alaninate;
propyl ((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-yl)carbamoyl)-2,3-
dihydrobenzo[b]thiophen-5-yl)methyl)(phenoxy)phosphoryl)-alaninate;
propyl ((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-yl)carbamoyl)-2,3-
dihydrobenzo[b]thiophen-5-yl)methyl)(phenoxy)phosphoryl)-alaninate;
propyl ((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(4,4,4-trifluorobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl (((2-((9-((3,3-difluoropropyl)(methyl)amino)-5-oxo-3-(6-phenyl-4-azaspiro[2.4]heptane-
4-carbonyl)decahydropyrrolo[1,2-a]azocin-6-yl)carbamoyl)-2,3-dihydrobenzo[b]thiophen-5-
yl)fluoromethyl)(phenoxy)phosphoryl)-alaninate;
propyl ((fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl (((2-((9-((3,3-difluoropropyl)(methyl)amino)-5-oxo-3-(6-phenyl-4-azaspiro[2.4]heptane-
4-carbonyl)decahydropyrrolo[1,2-a]azocin-6-yl)carbamoyl)benzo[b]thiophen-5-
yl)fluoromethyl)(phenoxy)phosphoryl)-alaninate;
propyl (((2-((9-((3,3-difluoropropyl)(methyl)amino)-5-oxo-3-(6-phenyl-4-azaspiro[2.4]heptane-
4-carbonyl)decahydropyrrolo[1,2-a]azocin-6-yl)carbamoyl)benzo[b]thiophen-5-
yl)fluoromethyl)(phenoxy)phosphoryl)-alaninate;
propyl ((fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
cyclobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
cyclobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl((oxetan-2-yl)methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-yl)carbamoyl)-2,3-
dihydrobenzo[b]thiophen-5-yl)methyl)(phenoxy)phosphoryl)-alaninate;
propyl ((fluoro(2-((9-(methyl((oxetan-2-yl)methyl)amino)-5-oxo-3-(6-phenyl-4-
azaspiro[2.4]heptane-4-carbonyl)decahydropyrrolo[1,2-a]azocin-6-yl)carbamoyl)-2,3-
dihydrobenzo[b]thiophen-5-yl)methyl)(phenoxy)phosphoryl)-alaninate;
propyl ((fluoro(2-((9-(methyl((oxetan-2-yl)methyl)amino)-5-oxo-3-(6-phenyl-4-
azaspiro[2.4]heptane-4-carbonyl)decahydropyrrolo[1,2-a]azocin-6-yl)carbamoyl)-2,3-
dihydrobenzo[b]thiophen-5-yl)methyl)(phenoxy)phosphoryl)-alaninate;
propyl ((fluoro(2-((9-(methyl(tetrahydrofuran-3-yl)amino)-5-oxo-3-(6-phenyl-4-
azaspiro[2.4]heptane-4-carbonyl)decahydropyrrolo[1,2-a]azocin-6-yl)carbamoyl)-2,3-
dihydrobenzo[b]thiophen-5-yl)methyl)(phenoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(tetrahydrofuran-3-yl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(oxetan-3-ylmethyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(tetrahydrofuran-3-yl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-((3-fluorocyclobutyl)(methyl)amino)-5-oxo-3-(6-(pyridin-3-yl)-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl ((fluoro(2-((9-(methyl(oxetan-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl (((2-((3-(6-(4-(dimethylamino)pyridin-3-yl)-4-azaspiro[2.4]heptane-4-carbonyl)-9-
morpholino-5-oxodecahydropyrrolo[1,2-a]azocin-6-yl)carbamoyl)benzo[b]thiophen-5-
yl)fluoromethyl)(phenoxy)phosphoryl)-alaninate;
propyl ((fluoro(2-((9-(methyl(tetrahydrofuran-3-yl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl ((fluoro(2-((9-(methyl(oxetan-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl ((fluoro(2-((9-(methyl(oxetan-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl ((fluoro(2-((3-(6-(3-fluorophenyl)-4-azaspiro[2.4]heptane-4-carbonyl)-9-(methyl(oxetan-
3-yl)amino)-5-oxodecahydropyrrolo[1,2-a]azocin-6-yl)carbamoyl)benzo[b]thiophen-5-
yl)methyl)(phenoxy)phosphoryl)-alaninate;
propyl ((fluoro(2-((9-(methyl(oxetan-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl (((2-((3-(7-cyano-6-phenyl-4-azaspiro[2.4]heptane-4-carbonyl)-9-morpholino-5-
oxodecahydropyrrolo[1,2-a]azocin-6-yl)carbamoyl)benzo[b]thiophen-5-
yl)fluoromethyl)(phenoxy)phosphoryl)-alaninate;
propyl ((fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl ((fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl ((fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl ((fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl ((fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl (((2-((3-(7-cyano-6-(pyridin-3-yl)-4-azaspiro[2.4]heptane-4-carbonyl)-9-morpholino-5-
oxodecahydropyrrolo[1,2-a]azocin-6-yl)carbamoyl)benzo[b]thiophen-5-
yl)fluoromethyl)(phenoxy)phosphoryl)-alaninate;
propyl (((2-((3-(7-cyano-6-(pyridin-3-yl)-4-azaspiro[2.4]heptane-4-carbonyl)-9-morpholino-5-
oxodecahydropyrrolo[1,2-a]azocin-6-yl)carbamoyl)benzo[b]thiophen-5-
yl)fluoromethyl)(phenoxy)phosphoryl)-alaninate;
propyl (((2-((3-(6-(4-(dimethylamino)pyridin-3-yl)-4-azaspiro[2.4]heptane-4-carbonyl)-9-
morpholino-5-oxodecahydropyrrolo[1,2-a]azocin-6-yl)carbamoyl)benzo[b]thiophen-5-
yl)fluoromethyl)(phenoxy)phosphoryl)-alaninate;
propyl (((2-((3-(6-(4-(dimethylamino)pyridin-3-yl)-4-azaspiro[2.4]heptane-4-carbonyl)-9-
morpholino-5-oxodecahydropyrrolo[1,2-a]azocin-6-yl)carbamoyl)benzo[b]thiophen-5-
yl)fluoromethyl)(phenoxy)phosphoryl)-alaninate;
propyl (((2-((3-(6-(4-(dimethylamino)pyridin-3-yl)-4-azaspiro[2.4]heptane-4-carbonyl)-9-
morpholino-5-oxodecahydropyrrolo[1,2-a]azocin-6-yl)carbamoyl)benzo[b]thiophen-5-
yl)fluoromethyl)(phenoxy)phosphoryl)-alaninate;
isopropyl ((fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
isopropyl ((fluoro(2-((9-morpholino-5-oxo-3-(6-(pyridin-3-yl)-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl ((fluoro(2-((9-(methyl((oxetan-2-yl)methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
benzyl (((2-((9-((3,3-difluoropropyl)(methyl)amino)-5-oxo-3-(6-phenyl-4-azaspiro[2.4]heptane-
4-carbonyl)decahydropyrrolo[1,2-a]azocin-6-yl)carbamoyl)benzo[b]thiophen-5-
yl)fluoromethyl)(phenoxy)phosphoryl)-alaninate;
propyl 2-((((2-((9-((3,3-difluoropropyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)amino)butanoate;
2-methoxyethyl (((2-((9-((3,3-difluoropropyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
propyl (((2-((9-(ethyl(3-fluorocyclobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)(phenoxy)phosphoryl)-alaninate;
propyl (((2-((9-(ethyl(3-fluorocyclobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)(phenoxy)phosphoryl)-alaninate;
isopropyl (((2-((9-((3,3-difluoropropyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
ethyl (((2-((9-((3,3-difluoropropyl)(methyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)(phenoxy)phosphoryl)-alaninate;
propyl ((fluoro(2-((9-(methyl(tetrahydrofuran-3-yl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(tetrahydrofuran-3-yl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
cyclobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
cyclobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
propyl (((2-((9-((3-cyanocyclobutyl)(methyl)amino)-5-oxo-3-(6-phenyl-4-azaspiro[2.4]heptane-
4-carbonyl)decahydropyrrolo[1,2-a]azocin-6-yl)carbamoyl)benzo[b]thiophen-5-
yl)fluoromethyl)(phenoxy)phosphoryl)-alaninate;
(3,3-difluorocyclobutyl)methyl ((fluoro(2-((9-((3-fluorocyclobutyl)(methyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
(3,3-difluorocyclobutyl)methyl ((fluoro(2-((9-((3-fluorocyclobutyl)(methyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isopropyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isopropyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl (((2-((9-((3-(difluoromethyl)cyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
propyl (((2-((9-((3-(difluoromethyl)cyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
3-fluorocyclobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
3-fluorocyclobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
2-ethylbutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
neopentyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
3-fluorocyclobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
3-fluorocyclobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isopropyl (ethoxy(fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphoryl)-alaninate;
2-ethylbutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
neopentyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(4,4,4-trifluorobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isopropyl (ethoxy(fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-((3-fluorocyclobutyl)(methyl)amino)-5-oxo-3-(4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
isopropyl (ethoxy(fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphoryl)-alaninate;
propyl ((fluoro(2-((9-(methyl(4,4,4-trifluorobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(propoxy)phosphoryl)-alaninate;
cyclobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
propyl (((2-((9-((3,3-difluorocyclopentyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(2,2,2-trifluoroethoxy)phosphoryl)-alaninate;
propyl ((fluoro(2-((9-(methyl(4,4,4-trifluorobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
propyl (cyclopropoxy(fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphoryl)-alaninate;
isopropyl (ethoxy(fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphoryl)-alaninate;
isopropyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
propyl (((2-((9-((3,3-difluorocyclopentyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(2,2,2-trifluoroethoxy)phosphoryl)-alaninate;
propyl ((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
propyl ((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
propyl (((2-((9-(ethyl((3-methyloxetan-3-yl)methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
propyl (((2-((9-(ethyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
propyl (((2-((9-(ethyl(4,4,4-trifluorobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)(phenoxy)phosphoryl)-alaninate;
propyl (((2-((9-(ethyl(4,4,4-trifluorobutyl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)fluoromethyl)(phenoxy)phosphoryl)-alaninate;
cyclobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
isopropyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
isobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isobutyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
cyclobutyl (ethoxy(fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphoryl)-alaninate;
cyclobutyl (ethoxy(fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphoryl)-alaninate;
propyl (((2-((9-(ethyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
propyl (((2-((9-(ethyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
propyl (((2-((9-((3-cyanocyclobutyl)(methyl)amino)-5-oxo-3-(6-phenyl-4-azaspiro[2.4]heptane-
4-carbonyl)decahydropyrrolo[1,2-a]azocin-6-yl)carbamoyl)benzo[b]thiophen-5-
yl)fluoromethyl)(2,2,2-trifluoroethoxy)phosphoryl)-alaninate;
cyclobutyl (((2-((9-((3-cyanocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
cyclobutyl (((2-((9-((3-cyanocyclobutyl) (methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
cyclobutyl ((fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
propyl ((fluoro(2-((9-(methyl(4,4,4-trifluorobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl (cyclopropoxy(fluoro(2-((9-(methyl(3-(trifluoromethoxy)cyclobutyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)phosphoryl)-alaninate;
propyl (cyclopropoxy(fluoro(2-((9-(methyl(3-(trifluoromethyl)cyclobutyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)phosphoryl)-alaninate;
isopropyl ((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl (((2-((9-(ethyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
propyl ((fluoro(2-((9-(methyl(4,4,4-trifluorobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isopropyl ((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate 2,2,2-
trifluoroacetate;
isobutyl (cyclopropoxy(fluoro(2-((9-(methyl(3-(trifluoromethoxy)cyclobutyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)phosphoryl)-alaninate;
cyclobutyl (ethoxy(fluoro(2-((9-(methyl(3-(trifluoromethoxy)cyclobutyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)phosphoryl)-alaninate;
cyclobutyl ((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
cyclobutyl ((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl N-((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-O-methyl-serinate;
propyl 2-(((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)amino)-2-methylpropanoate;
propyl ((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-phenylalaninate;
isobutyl ((fluoro(2-((9-(methyl((3-(trifluoromethyl)cyclobutyl)methyl)amino)-5-oxo-3-(6-phenyl-
4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isobutyl (((2-((9-(((3-(difluoromethyl)cyclobutyl)methyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
isobutyl ((fluoro(2-((9-(methyl(2-(1-(trifluoromethyl)cyclopropyl)ethyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isobutyl ((fluoro(2-((9-(methyl(3-(trifluoromethoxy)cyclobutyl)amino)-5-oxo-3-(4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isobutyl ((fluoro(2-((9-(methyl(3-(trifluoromethoxy)propyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isobutyl ((fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isobutyl ((fluoro(2-((9-(methyl(4,4,4-trifluorobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isobutyl ((fluoro(2-((9-(methyl(4,4,4-trifluorobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(4,4,4-trifluoro-3-methoxybutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl (((2-((9-(((3-(difluoromethyl)cyclobutyl)methyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
propyl ((fluoro(2-((9-(methyl(2-(1-(trifluoromethyl)cyclopropyl)ethyl)amino)-5-oxo-3-(6-phenyl-
4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(3-(trifluoromethoxy)propyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(3-(trifluoromethoxy)propyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl (((2-((9-((3,3-difluorocyclopentyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
propyl (((2-((9-((3,3-difluorocyclopentyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
propyl ((fluoro(2-((9-(methyl(4,4,4-trifluorobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-morpholino-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
propyl ((fluoro(2-((9-(methyl((3-methyloxetan-3-yl)methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(oxetan-3-yl)amino)-5-oxo-3-(6-phenyl-4-azaspiro[2.4]heptane-4-
carbonyl)decahydropyrrolo[1,2-a]azocin-6-yl)carbamoyl)benzo[b]thiophen-5-
yl)methyl)(phenoxy)phosphoryl)-alaninate;
isobutyl (((2-((9-((3,3-difluoropropyl)(methyl)amino)-5-oxo-3-(6-phenyl-4-azaspiro[2.4]heptane-
4-carbonyl)decahydropyrrolo[1,2-a]azocin-6-yl)carbamoyl)benzo[b]thiophen-5-
yl)fluoromethyl)(phenoxy)phosphoryl)-alaninate;
propyl N-(((2-((9-((3,3-difluoropropyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-O-methyl-serinate;
propyl 2-((((2-((9-((3,3-difluoropropyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)amino)-2-
methylpropanoate;
isopropyl 2-((((2-((9-((3,3-difluoropropyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)amino)butanoate;
propyl ((difluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)prolinate;
propyl 1-(ethoxy(fluoro(2-((9-(methyl(3-(trifluoromethoxy)cyclobutyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)phosphoryl)-3,3-difluoropyrrolidine-2-carboxylate;
propyl ((difluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
dipropyl 2,2'-(((fluoro(2-((9-(methyl(3-(trifluoromethoxy)cyclobutyl)amino)-5-oxo-3-(6-phenyl-
4-azaspiro[2.4]heptane-4-carbonyl)decahydropyrrolo[1,2-a]azocin-6-
yl)carbamoyl)benzo[b]thiophen-5-yl)methyl)phosphoryl)bis(azanediyl))dipropionate;
isobutyl (cyclopropoxy(fluoro(2-((9-(methyl(3-(trifluoromethoxy)cyclobutyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)phosphoryl)-alaninate;
propyl 1-(((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)amino)cyclopropane-1-
carboxylate;
isobutyl ((fluoro(2-((9-(methyl(3-(trifluoromethoxy)cyclopentyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isobutyl ((fluoro(2-((9-(methyl(3-(trifluoromethoxy)cyclopentyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(3-(trifluoromethoxy)cyclopentyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
(1-fluorocyclobutyl)methyl ((fluoro(2-((9-((3-fluorocyclobutyl)(methyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(3,3,3-trifluoropropoxy)phosphoryl)-alaninate;
propyl ((fluoro(2-((9-(methyl(3-(trifluoromethoxy)cyclobutyl)amino)-5-oxo-3-(6-(pyridin-3-yl)-
4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(3-(trifluoromethoxy)cyclobutyl)amino)-5-oxo-3-(6-(pyridin-3-yl)-
4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((3-(7-methoxy-6-phenyl-4-azaspiro[2.4]heptane-4-carbonyl)-9-(methyl(3-
(trifluoromethoxy)cyclobutyl)amino)-5-oxodecahydropyrrolo[1,2-a]azocin-6-
yl)carbamoyl)benzo[b]thiophen-5-yl)methyl)(phenoxy)phosphoryl)-alaninate;
propyl ((fluoro(2-((3-(7-methoxy-6-phenyl-4-azaspiro[2.4]heptane-4-carbonyl)-9-(methyl(3-
(trifluoromethoxy)cyclobutyl)amino)-5-oxodecahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(3,3,3-trifluoro-2-methoxypropyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(2-(trifluoromethoxy)ethyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(2-(trifluoromethoxy)ethyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2-oxopyrrolidin-1-yl)phosphoryl)-alaninate;
cyclobutyl ((fluoro(2-((9-(methyl((3-methyloxetan-3-yl)methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl((3-methyloxetan-3-yl)methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
(tetrahydrofuran-3-yl)methyl ((fluoro(2-((9-(methyl(3-(trifluoromethyl)cyclobutyl)amino)-5-oxo-
3-(6-phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
isopropyl (ethoxy(fluoro(2-((9-(methyl(3-(trifluoromethyl)cyclobutyl)amino)-5-oxo-3-(6-phenyl-
4-azaspiro[2.4]heptane-4-carbonyl)decahydropyrrolo[1,2-a]azocin-6-
yl)carbamoyl)benzo[b]thiophen-5-yl)methyl)phosphoryl)-alaninate;
isopropyl (ethoxy(fluoro(2-((9-(methyl(4,4,4-trifluorobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphoryl)-alaninate;
propyl (ethoxy((2-((9-(ethyl(3-fluorocyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)phosphoryl)-alaninate;
2-morpholinoethyl (((2-((9-((3,3-difluoropropyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
neopentyl (((2-((9-((3,3-difluoropropyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)fluoromethyl)(phenoxy)phosphoryl)-alaninate;
propyl (((2-((9-((3,3-difluoropropyl)(methyl)amino)-5-oxo-3-(6-phenyl-4-azaspiro[2.4]heptane-
4-carbonyl)decahydropyrrolo[1,2-a]azocin-6-yl)carbamoyl)benzo[b]thiophen-5-
yl)fluoromethyl)(phenoxy)phosphoryl)-prolinate;
2-methoxy-2-methylpropyl ((fluoro(2-((9-((3-fluorocyclobutyl)(methyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
6,6-difluorospiro[3.3]heptan-2-yl ((fluoro(2-((9-((3-fluorocyclobutyl)(methyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
(tetrahydrofuran-3-yl)methyl ((fluoro(2-((9-((3-fluorocyclobutyl)(methyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
(tetrahydrofuran-3-yl)methyl ((fluoro(2-((9-((3-fluorocyclobutyl)(methyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
(tetrahydro-2H-pyran-4-yl)methyl ((fluoro(2-((9-((3-fluorocyclobutyl)(methyl)amino)-5-oxo-3-
(6-phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
isobutyl 2-(((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)amino)butanoate;
isopentyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl 2-(((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)amino)pentanoate;
propyl ((cyclohexyloxy)(fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphoryl)-alaninate;
propyl ((cyclopentyloxy)(fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(isopropoxy)phosphoryl)-alaninate;
neopentyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2-methoxyethoxy)phosphoryl)-alaninate;
spiro[3.3]heptan-2-yl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-valinate;
tetrahydrofuran-3-yl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
tetrahydrofuran-3-yl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
2-fluoro-2-methylpropyl ((fluoro(2-((9-((3-fluorocyclobutyl)(methyl)amino)-5-oxo-3-(6-phenyl-
4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
(tetrahydrofuran-3-yl)methyl ((fluoro(2-((9-((3-fluorocyclobutyl)(methyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
(tetrahydrofuran-3-yl)methyl ((fluoro(2-((9-((3-fluorocyclobutyl)(methyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
(tetrahydro-2H-pyran-4-yl)methyl ((fluoro(2-((9-((3-fluorocyclobutyl)(methyl)amino)-5-oxo-3-
(6-phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl 4,4-difluoro-1-((fluoro(2-((9-(methyl(3-(trifluoromethoxy)cyclobutyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)(phenoxy)phosphoryl)pyrrolidine-2-carboxylate;
propyl N-((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(2,2,2-trifluoroethoxy)phosphoryl)-N-methyl-
alaninate;
2-ethylbutyl (ethoxy(fluoro(2-((9-(methyl(3-(trifluoromethoxy)cyclobutyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)phosphoryl)-alaninate;
propyl N-(ethoxy(fluoro(2-((9-(methyl(3-(trifluoromethoxy)cyclobutyl)amino)-5-oxo-3-(6-
phenyl-4-azaspiro[2.4]heptane-4-carbonyl)decahydropyrrolo[1,2-a]azocin-6-
yl)carbamoyl)benzo[b]thiophen-5-yl)methyl)phosphoryl)-N-methyl-alaninate;
propyl (ethoxy(fluoro(2-((9-(methyl(3-(trifluoromethoxy)cyclobutyl)amino)-5-oxo-3-(6-phenyl-
4-azaspiro[2.4]heptane-4-carbonyl)decahydropyrrolo[1,2-a]azocin-6-
yl)carbamoyl)benzo[b]thiophen-5-yl)methyl)phosphoryl)-prolinate;
spiro[3.4]octan-2-yl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
spiro[2.3]hexan-5-yl ((fluoro(2-((9-((3-fluorocyclobutyl)(methyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate;
propyl ((fluoro(2-((9-(methyl(3-(trifluoromethyl)cyclobutyl)amino)-5-oxo-3-(6-phenyl-4-
azaspiro[2.4]heptane-4-carbonyl)decahydropyrrolo[1,2-a]azocin-6-
yl)carbamoyl)benzo[b]thiophen-5-yl)methyl)(phenoxy)phosphoryl)-alaninate,
propyl 2-(((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-yl)carbamoyl)-2,3-
dihydrobenzo[b]thiophen-5-yl)methyl)(isobutoxy)phosphoryl)amino)-2-methylpropanoate;
propyl 2-(((fluoro(2-((9-(methyl(3-(trifluoromethoxy)cyclobutyl)amino)-5-oxo-3-((S)-6-phenyl-
4-azaspiro[2.4]heptane-4-carbonyl)decahydropyrrolo[1,2-a]azocin-6-yl)carbamoyl)-2,3-
dihydrobenzo[b]thiophen-5-yl)methyl)(isopropoxy)phosphoryl)amino)-2-methylpropanoate;
propyl 2-((butoxy(fluoro(2-((9-(methyl(3-(trifluoromethoxy)cyclobutyl)amino)-5-oxo-3-(6-
phenyl-4-azaspiro[2.4]heptane-4-carbonyl)decahydropyrrolo[1,2-a]azocin-6-yl)carbamoyl)-2,3-
dihydrobenzo[b]thiophen-5-yl)methyl)phosphoryl)amino)-2-methylpropanoate;
propyl 2-(((fluoro(2-((9-(methyl(3-(trifluoromethoxy)cyclobutyl)amino)-5-oxo-3-(6-phenyl-4-
azaspiro[2.4]heptane-4-carbonyl)decahydropyrrolo[1,2-a]azocin-6-yl)carbamoyl)-2,3-
dihydrobenzo[b]thiophen-5-yl)methyl)(propoxy)phosphoryl)amino)-2-methylpropanoate; and
propyl 2-((ethoxy(fluoro(2-((9-(methyl(3-(trifluoromethoxy)cyclobutyl)amino)-5-oxo-3-(6-
phenyl-4-azaspiro[2.4]heptane-4-carbonyl)decahydropyrrolo[1,2-a]azocin-6-yl)carbamoyl)-2,3-
dihydrobenzo[b]thiophen-5-yl)methyl)phosphoryl)amino)-2-methylpropanoate;

and pharmaceutically acceptable salts thereof.

In some embodiments, the compound, or a pharmaceutically acceptable salt thereof, is selected from the group consisting of parent drugs A3, A4, A5, A6, A7, A8, A11, A13, A14, A18, A19, A20, A21, A22, A23, A25, A30, A32, A33, A35, A36, A37, A38, A40, A42, A43, A44, A47, A48, A49, A50, A52, A53, A55, A57, A58, A59, A60, A61, and A65, and pharmaceutically acceptable salts thereof. In some embodiments, the compound, or a pharmaceutically acceptable salt thereof, is selected from the group consisting of compounds A3, A4, A5, A6, A7, A8, A11, A13, A14, A18, A19, A20, A21, A22, A23, A25, A30, A32, A33, A35, A36, A37, A38, A40, A42, A43, A44, A47, A48, A49, A50, A52, A53, A55, A57, A58, A59, A60, A61, and A65, prodrugs or intermediate metabolites thereof, and pharmaceutically acceptable salts of any of the foregoing.

In some embodiments, the compound, or a pharmaceutically acceptable salt thereof, is selected from the group consisting of prodrugs C4, C5, C6, C7, C8, C10, C13, C17, C19, C23, C25, C53, C54, C61, C63, C64, C65, C66, C68, C70, C71, C73, C74, C75, C82, C83, C84, C89, C90, C91, C92, C93, C94, C96, C97, C96, C97, C102, C103, C015, C016, C017, C110, C111, C114, C116, C118, C121, C122, C123, C124, C125, C126, C127, C132, C133, C135, C136, C137, C140, C141, C143, C144, C148, C155, C156, C160, C162, C170, C173, C175, C176, C177, C180, C182, C185, C186, C188, C189, C194, C195, C197, C198, C205, C208, and C209, and pharmaceutically acceptable salts thereof.

Any variation or embodiment of R^{1a}, R^{1b}, R², R³, R⁴, R^{4a}, R^{4b}, R⁵, R^{P}, R^{P1}, R^{P2}, R^{Pa}, R^{PE}, R^{Paa1}, R^{Paa2}, R^{Paa3}, R^{Paa4}, or Ring A provided herein can be combined with every other variation or embodiment of R^{1a}, R^{1b}, R², R³, R⁴, R^{4a}, R^{4b}, R⁵, R^{P}, R^{P1}, R^{P2}, R^{Pa}, R^{PE}, R^{Paa1}, R^{Paa2}, R^{Paa3}, R^{Paa4}, or Ring A the same as if each and every combination had been individually and specifically described.

As used herein, when any variable occurs more than one time in a chemical formula, its definition on each occurrence is independent of its definition at every other occurrence.

### Methods of Synthesis

Compounds of Formula (I), and those described herein may be prepared in various ways as generally described below and more specifically in the Examples hereinafter (such as the schemes provided in the Examples below). General synthetic routes to compounds of Formula (I), and some examples of starting materials used to synthesize compounds of Formula (I) are shown and described herein. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

Certain stereochemical centers have been left unspecified and certain substituents have been eliminated in the following schemes for the sake of clarity and are not intended to limit the teaching of the schemes in any way. Furthermore, individual isomers, enantiomers, and diastereomers may be separated or resolved by one of ordinary skill in the art at any convenient point in the synthesis of compounds of the invention, by methods such as selective crystallization techniques or chiral chromatography (See for example, J. Jacques, et al., “Enantiomers, Racemates, and Resolutions”, John Wiley and Sons, Inc., 1981, and E. L. Eliel and S. H. Wilen, “Stereochemistry of Organic Compounds”, Wiley-Interscience, 1994).

Compounds of Formula (I), (I-A-1), (I-A-2), (I-A-3), (I-A-4), (I-A-5), (I-A-6), (I-B-1), (I-B-2), (I-B-3), (I-B-4), (I-B-5), (I-B-6), (I-C-1), (I-C-2), (I-C-3), (I-C-4), (I-C-5), (I-C-6), (I-D-1), (I-D-2), (I-D-3), (I-D-4), (I-D-5), (I-D-6), (I-E-1), (I-E-2), (I-E-3), (I-E-4), (I-E-5), (I-E-6), (I-F-1), (I-F-2), (I-F-3), (I-F-4), (I-F-5), (I-F-6), (I-G-1), (I-G-2), (I-G-3), (I-G-4), (I-G-5), (I-G-6), (I-H-1), (I-H-2), (I-H-3), (I-H-4), (I-H-5), (I-H-6), (I-J-1), (I-J-2), (I-J-3), (I-J-4), (I-J-5), or (I-J-6) can be prepared according to Scheme A, Scheme B, Scheme C, Scheme D, Scheme E, or Scheme F, wherein the R^{1a}, R^{1b}, R², R³, R⁴, R^{4a}, R^{4b}, R⁵, R^{P}, R^{Pa}, R^{Paa2}, R^{Paa3}, and R^{Paa4}, are as defined for Formula (I) or any applicable variations thereof as detailed herein.

Methods for preparing compound (A) or a salt of thereof, are shown herein, as shown in Scheme A. The method of preparing compound (A) can be applied to prepare the starting material for Schemes B—F.

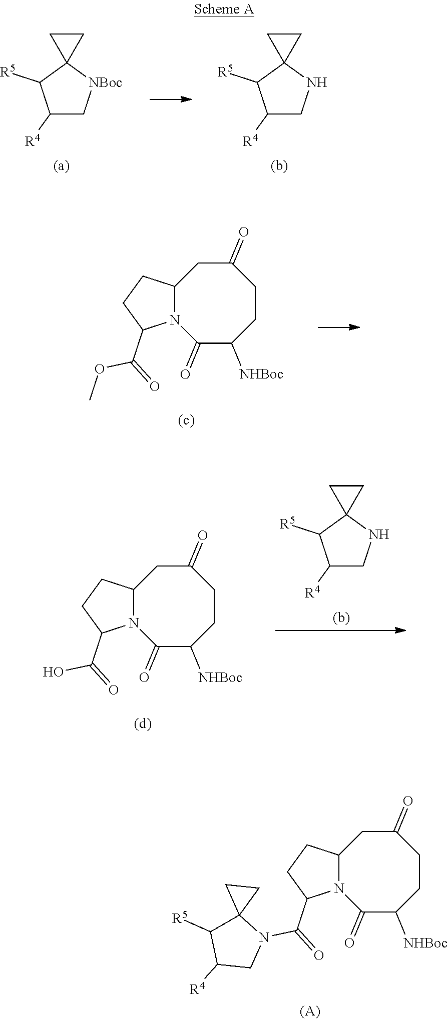

In one aspect, provided herein is a method of preparing a compound of formula (A),

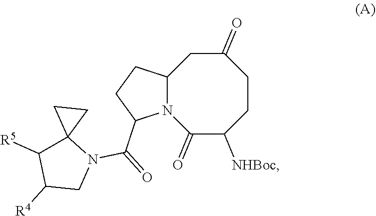

or a salt thereof, comprising contacting a compound of formula (d)

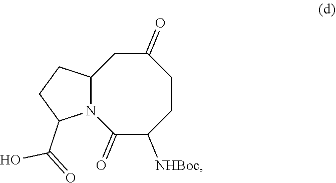 [SMILES: CC(C)(C)OC(=O)NC1CCC(=O)CC2CCC(C(=O)O)N2C1=O]

or a salt thereof, with a compound of formula (b),

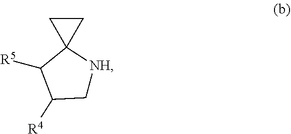

or a salt thereof, in the presence of a coupling reagent and an aliphatic amine. In some embodiments, the coupling reagent comprises O-(7-Azabenzotriazol-1-yl)-N,N,N′,N′-tetramethyluronium hexafluorophosphate (HATU). In some embodiments, the aliphatic amine comprises triethylamine. In some embodiments, the contacting of the compound of formula (d) or a salt thereof with a compound of formula (b) or a salt thereof is carried out at a temperature between about 20° C. and about 30° C. In some embodiments, the contacting of the compound of formula (d) or a salt thereof with a compound of formula (b) or a salt thereof is carried out at a temperature between about 20° C. and about 25° C. In some embodiments, the contacting of the compound of formula (d) or a salt thereof with a compound of formula (b) or a salt thereof is carried out at a temperature of about 25° C. In some embodiments, the contacting of the compound of formula (d) or a salt thereof with a compound of formula (b) or a salt thereof is carried out for about one hour to five hours. In some embodiments, the contacting of the compound of formula (d) or a salt thereof with a compound of formula (b) or a salt thereof is carried out for about one hour to three hours. In some embodiments, the contacting of the compound of formula (d) or a salt thereof with a compound of formula (b) or a salt thereof is carried out for about two hours.

In some embodiments, the compound of Formula (d) or a salt thereof is prepared by contacting a compound of Formula (c),

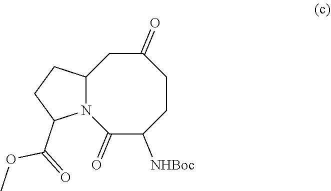 [SMILES: COC(=O)C1CCC2CC(=O)CCC(NC(=O)OC(C)(C)C)C(=O)N21]

or a salt thereof, with a strong base in the presence of a polar aprotic solvent. In some embodiments, the strong base comprises a metal hydroxide. In some embodiments, the strong base comprises LiOH·H₂O. In some embodiments, the polar aprotic solvent comprises tetrahydrofuran. In some embodiments, the contacting of the compound of Formula (c) or a salt thereof with a strong base in the presence of a polar aprotic solvent is carried out at a temperature between about 10° C. and 30° C. In some embodiments, the contacting of the compound of Formula (c) or a salt thereof with a strong base in the presence of a polar aprotic solvent is carried out at a temperature between about 15° C. and 25° C. In some embodiments, the contacting of the compound of Formula (c) or a salt thereof with a strong base in the presence of a polar aprotic solvent is carried out at a temperature of about 20° C. In some embodiments, the contacting of the compound of Formula (c) or a salt thereof with a strong base in the presence of a polar aprotic solvent is carried out for about one hour to five hours. In some embodiments, the contacting of the compound of Formula (c) or a salt thereof with a strong base in the presence of a polar aprotic solvent is carried out for about one hour to three hours. In some embodiments, the contacting of the compound of Formula (c) or a salt thereof with a strong base in the presence of a polar aprotic solvent is carried out for about two hours.

In one aspect, provided herein is a method of preparing a compound of formula

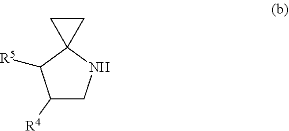

or a salt thereof, comprising contacting a compound of Formula (a),

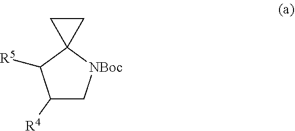

or a salt thereof with a strong organic acid. In some embodiments, the strong organic acid comprises trifluoroacetic acid. In some embodiments, the contacting of the compound of Formula (a) or a salt thereof is carried out at a temperature between about 20° C. and about 30° C. In some embodiments, the contacting of the compound of Formula (a) or a salt thereof is carried out at a temperature between about 20° C. and about 25° C. In some embodiments, the contacting of the compound of Formula (a) or a salt thereof is carried out at a temperature of about 25° C. In some embodiments, the contacting of the compound of Formula (a) or a salt thereof is carried out for about one hour to five hours. In some embodiments, the contacting of the compound of Formula (a) or a salt thereof is carried out for about one hour to three hours. In some embodiments, the contacting of the compound of Formula (a) or a salt thereof is carried out for about two hours.

Methods for preparing compound (D) or a salt of thereof, are shown herein, as shown in Scheme B. The method of preparing compound (D) can be applied to prepare the starting material for Schemes C—F.

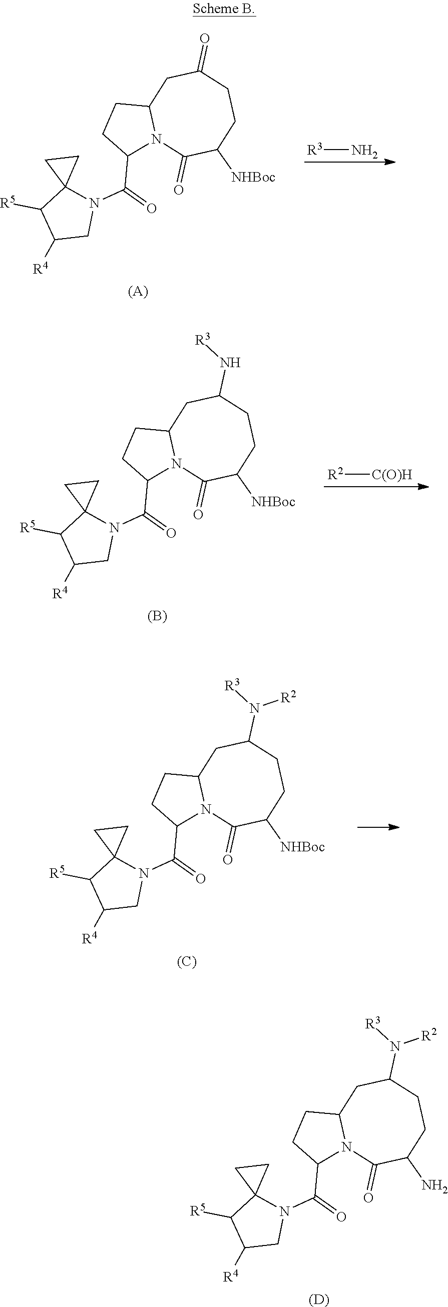

In some embodiments, the compound of Formula (D) or a salt thereof is prepared by contacting a compound of Formula (C),

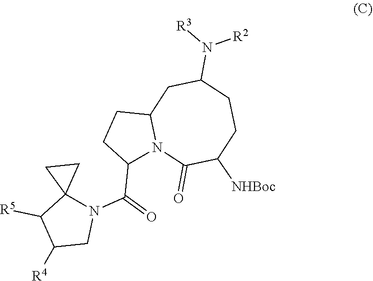

or a salt thereof, with a strong organic acid and a polar aprotic solvent. In some embodiments, the strong organic acid comprises trifluoroacetic acid. In some embodiments, the polar aprotic solvent comprises dichloromethane. In some embodiments, the contacting of the compound of Formula (C) or a salt thereof is carried out at a temperature between about 20° C. and about 30° C. In some embodiments, the contacting of the compound of Formula (C) or a salt thereof is carried out at a temperature between about 20° C. and about 25° C. In some embodiments, the contacting of the compound of Formula (C) or a salt thereof is carried out at a temperature of about 25° C. In some embodiments, the contacting of the compound of Formula (C) or a salt thereof is carried out for about 30 minutes to two hours. In some embodiments, the contacting of the compound of Formula (C) or a salt thereof is carried out for about one hour to two hours. In some embodiments, the contacting of the compound of Formula (C) or a salt thereof is carried out for about one hour.

In some embodiments, the compound of Formula (C) or a salt thereof is prepared by contacting a compound of Formula (B),

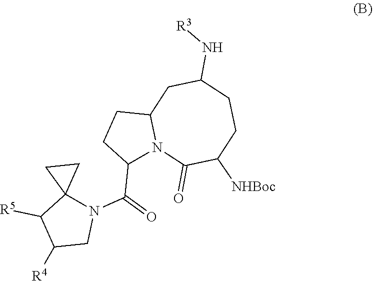

or a salt thereof, with R²—C(O)H and one or more polar protic solvents. In some embodiments, the polar protic solvents comprise methanol or sodium borohydride, or both. In some embodiments, the contacting of the compound of Formula (B) or a salt thereof is carried out at a temperature between about 20° C. and about 30° C. In some embodiments, the contacting of the compound of Formula (B) or a salt thereof is carried out at a temperature between about 20° C. and about 25° C. In some embodiments, the contacting of the compound of Formula (B) or a salt thereof is carried out at a temperature of about 25° C.

In some embodiments, the compound of Formula (B) or a salt thereof is prepared by contacting a compound of Formula (A),

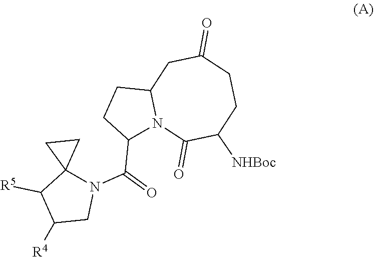

or a salt thereof, with R³—NH₂. In some embodiments, the contacting of the compound of Formula (A) or a salt thereof with R³—NH₂ is carried out in the presence of a catalyst, a polar protic solvent, and a mild reducing agent. In some embodiments, the catalyst is a metal halide. In some embodiments, the catalyst comprises zinc chloride. In some embodiments, the polar protic solvent comprises methanol. In some embodiments, the mild reducing agent comprises sodium cyanoborohydride. In some embodiments, the contacting of the compound of Formula (A) or a salt thereof with R³—NH₂ is carried out at a temperature between about 40° C. and about 60° C. In some embodiments, the contacting of the compound of Formula (A) or a salt thereof with R³—NH₂ is carried out at a temperature between about 50° C. and about 60° C. In some embodiments, the contacting of the compound of Formula (A) or a salt thereof with R³—NH₂ is carried out at a temperature of about 55° C. to 15 hours. In some embodiments, the contacting of the compound of Formula (A) or a salt thereof with R³—NH₂ is carried out for about 11 to 14 hours. In some embodiments, the contacting of the compound of Formula (A) or a salt thereof with R³—NH₂ is carried out for about 12 hours.

Methods for preparing a parent compound, intermediate metabolite, or a prodrug, or a salt of any of the foregoing are shown herein, as shown in Scheme C.

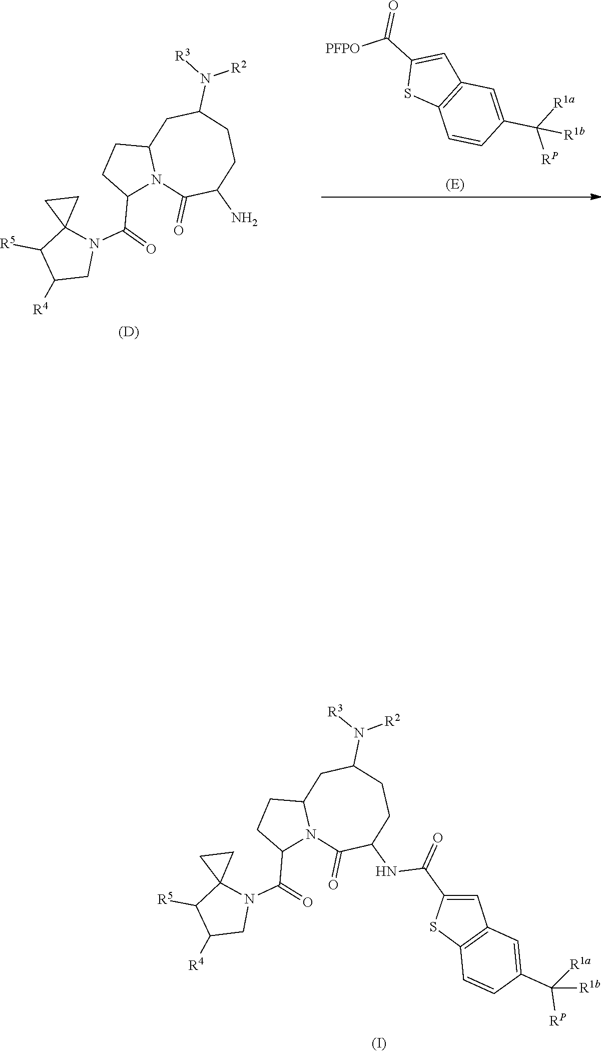

In one aspect, provided herein is a method of preparing a compound of formula

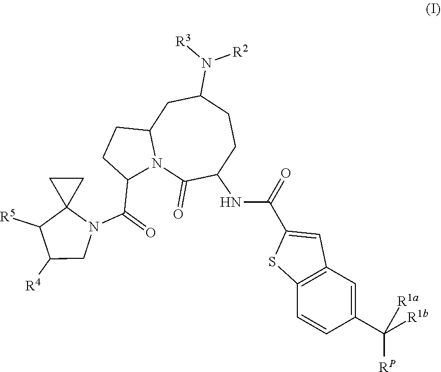

or a salt thereof, comprising contacting a compound of Formula (D),

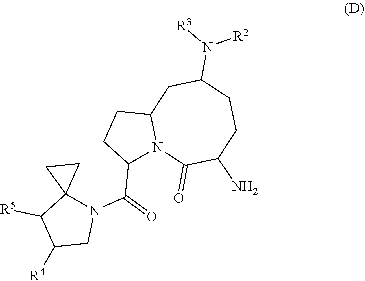

or a salt thereof with a compound of Formula (E),

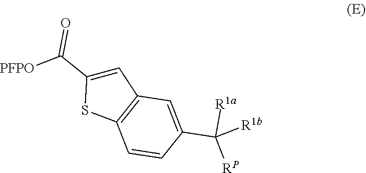

or a salt thereof. PFP is perfluorophenyl. In some embodiments, the contacting of the compound of Formula (D) or a salt thereof with the compound of Formula (E) or a salt thereof is carried out in the presence of a polar aprotic solvent and an aliphatic amine. In some embodiments, the polar aprotic solvent comprises dimethylformamide. In some embodiments, the aliphatic amine comprises triethylamine. In some embodiments, the contacting of the compound of Formula (D) or a salt thereof with the compound of Formula (E) or a salt thereof is carried out at a temperature between about 20° C. and about 30° C. In some embodiments, the contacting of the compound of Formula (D) or a salt thereof with the compound of Formula (E) or a salt thereof is carried out at a temperature between about 20° C. and about 25° C. In some embodiments, the contacting of the compound of Formula (D) or a salt thereof with the compound of Formula (E) or a salt thereof is carried out at a temperature of about 25° C. In some embodiments, the contacting of the compound of Formula (D) or a salt thereof with the compound of Formula (E) or a salt thereof is carried out for about 30 minutes to two hours. In some embodiments, the contacting of the compound of Formula (D) or a salt thereof with the compound of Formula (E) or a salt thereof is carried out for about one hour to two hours. In some embodiments, the contacting of the compound of Formula (D) or a salt thereof with the compound of Formula (E) or a salt thereof is carried out for about one hour.

Methods for preparing a parent compound or a salt thereof are shown herein, as shown in Scheme D.

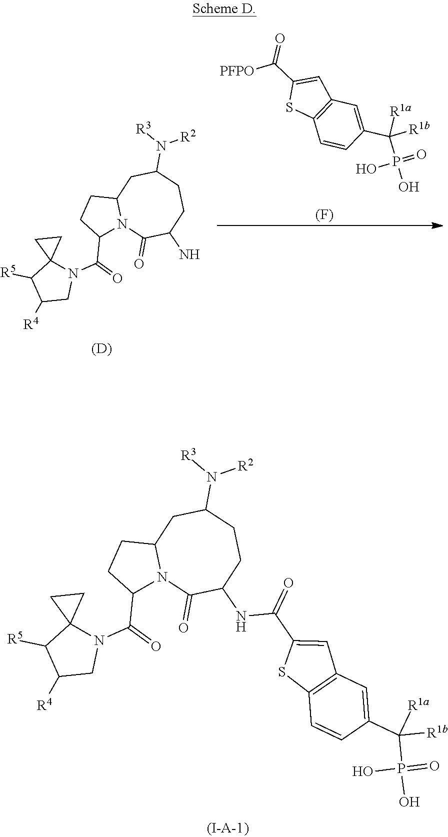

In one aspect, provided herein is a method of preparing a parent compound of formula

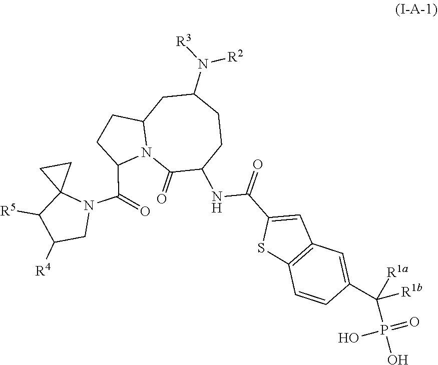

or a salt thereof, comprising contacting a compound of Formula (D)

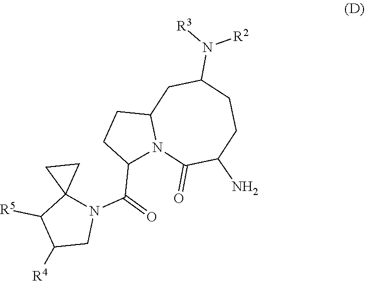

or a salt thereof with a compound of Formula (F),

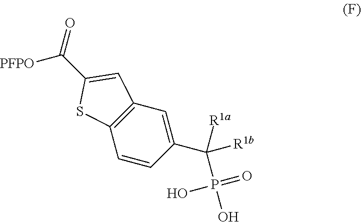

or a salt thereof. In some embodiments, the contacting of the compound of Formula (D) or a salt thereof with the compound of Formula (F) or a salt thereof is carried out in the presence of a polar aprotic solvent and an aliphatic amine. In some embodiments, the polar aprotic solvent comprises dimethylformamide. In some embodiments, the aliphatic amine comprises triethylamine. In some embodiments, the contacting of the compound of Formula (D) or a salt thereof with the compound of Formula (F) or a salt thereof is carried out at a temperature between about 20° C. and about 30° C. In some embodiments, the contacting of the compound of Formula (D) or a salt thereof with the compound of Formula (F) or a salt thereof is carried out at a temperature between about 20° C. and about 25° C. In some embodiments, the contacting of the compound of Formula (D) or a salt thereof with the compound of Formula (F) or a salt thereof is carried out at a temperature of about 25° C. In some embodiments, the contacting of the compound of Formula (D) or a salt thereof with the compound of Formula (F) or a salt thereof is carried out for about 30 minutes to two hours. In some embodiments, the contacting of the compound of Formula (D) or a salt thereof with the compound of Formula (F) or a salt thereof is carried out for about one hour to two hours. In some embodiments, the contacting of the compound of Formula (D) or a salt thereof with the compound of Formula (F) or a salt thereof is carried out for about one hour.

Methods for preparing a prodrug or intermediate metabolite or a salt thereof are shown herein, as shown in Scheme E.

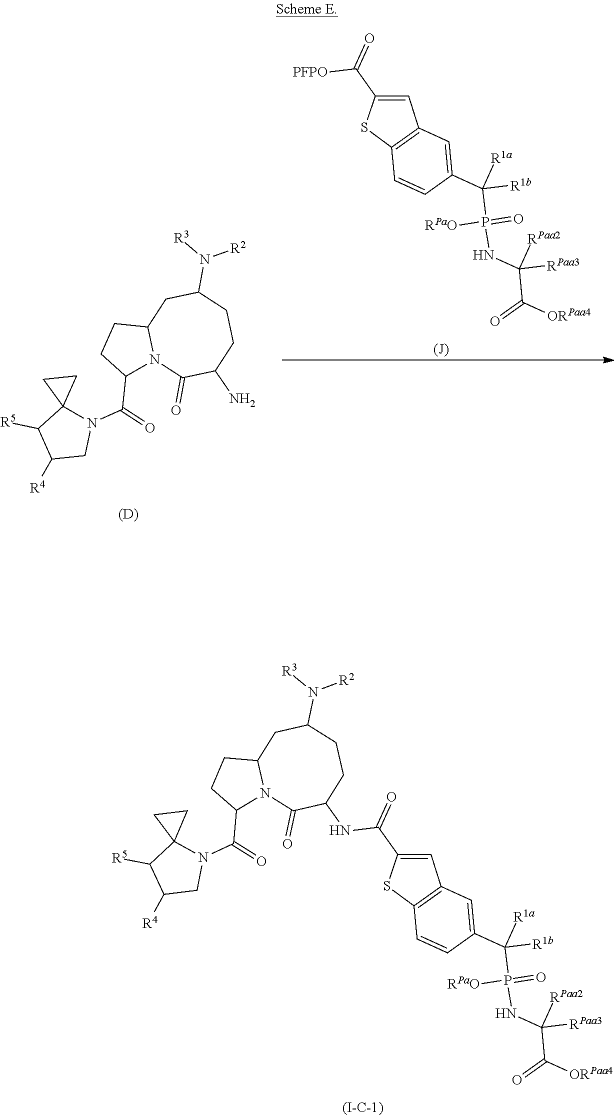

In some embodiments, provided herein is a method of preparing a prodrug of the formula

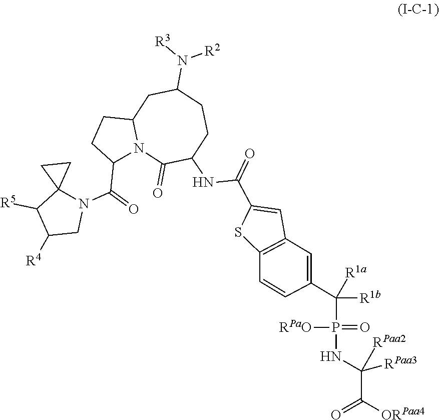

or a salt thereof, comprising contacting a compound of Formula (D),

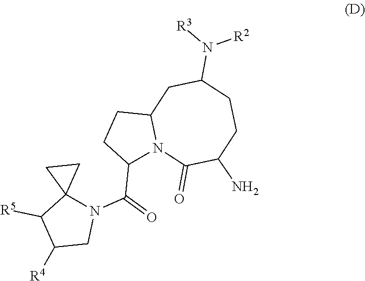

or a salt thereof, with a compound of Formula (J)

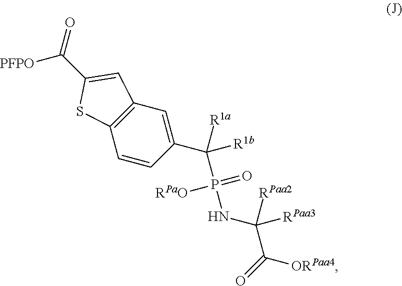

or a salt thereof. In some embodiments, the contacting of the compound of Formula (D) or a salt thereof with the compound of Formula (J) or a salt thereof is carried out in the presence of a polar aprotic solvent and an aliphatic amine. In some embodiments, the polar aprotic solvent comprises dimethylformamide. In some embodiments, the aliphatic amine comprises triethylamine. In some embodiments, the contacting of the compound of Formula (D) or a salt thereof with the compound of Formula (J) or a salt thereof is carried out at a temperature between about 20° C. and about 30° C. In some embodiments, the contacting of the compound of Formula (D) or a salt thereof with the compound of Formula (J) or a salt thereof is carried out at a temperature between about 20° C. and about 25° C. In some embodiments, the contacting of the compound of Formula (D) or a salt thereof with the compound of Formula (J) or a salt thereof is carried out at a temperature of about 25° C. In some embodiments, the contacting of the compound of Formula (D) or a salt thereof with the compound of Formula (J) or a salt thereof is carried out for about 30 minutes to two hours. In some embodiments, the contacting of the compound of Formula (D) or a salt thereof with the compound of Formula (J) or a salt thereof is carried out for about one hour to two hours. In some embodiments, the contacting of the compound of Formula (D) or a salt thereof with the compound of Formula (F) or a salt thereof is carried out for about one hour.

Methods for preparing an intermediate metabolite or a salt thereof are shown herein, as shown in Scheme F.

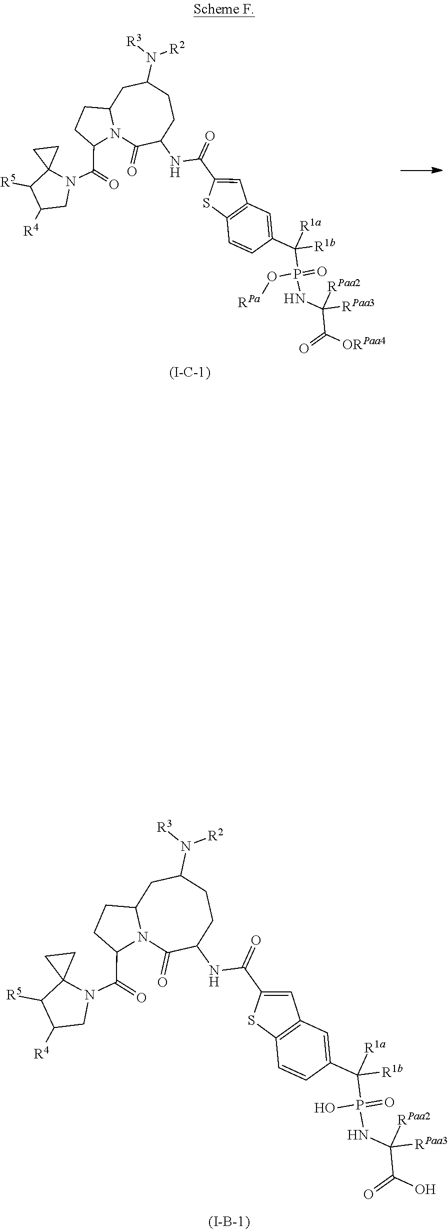

In some embodiments, provided herein is a method of preparing an intermediate metabolite of the formula

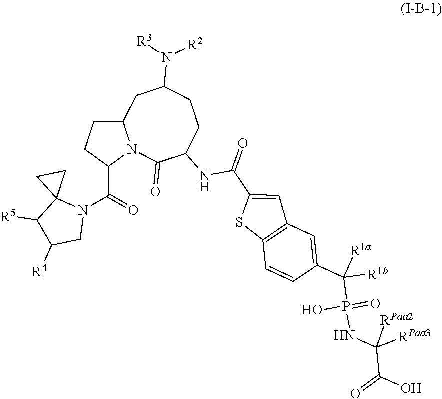

or a salt thereof, comprising contacting a compound of Formula (I-C-1),

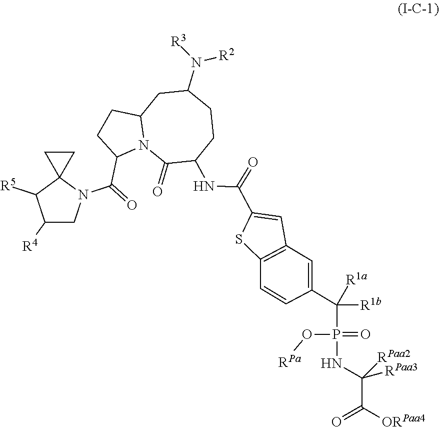

or a salt thereof, with a strong base in the presence of a polar aprotic solvent. In some embodiments, the strong base comprises a metal hydroxide. In some embodiments, the strong base comprises LiOH—H₂O. In some embodiments, the polar aprotic solvent comprises tetrahydrofuran. In some embodiments, the contacting of the compound of Formula (I-C-1) or a salt thereof with a strong base in the presence of a polar aprotic solvent is carried out at a temperature between about 10° C. and 30° C. In some embodiments, the contacting of the compound of Formula (I-C-1) or a salt thereof with a strong base in the presence of a polar aprotic solvent is carried out at a temperature between about 15° C. and 25° C. In some embodiments, the contacting of the compound of Formula (I-C-1) or a salt thereof with a strong base in the presence of a polar aprotic solvent is carried out at a temperature of about 20° C. In some embodiments, the contacting of the compound of Formula (I-C-1) or a salt thereof with a strong base in the presence of a polar aprotic solvent is carried out for about one hour to five hours. In some embodiments, the contacting of the compound of Formula (I-C-1) or a salt thereof with a strong base in the presence of a polar aprotic solvent is carried out for about one hour to three hours. In some embodiments, the contacting of the compound of Formula (I-C-1) or a salt thereof with a strong base in the presence of a polar aprotic solvent is carried out for about two hours.

### Uses, Formulation, and Administration

In one aspect, provided herein is a pharmaceutical composition comprising a compound of Formula (I) or a prodrug thereof, or a pharmaceutically acceptable salt thereof, as an active ingredient. Such a pharmaceutical composition comprises a therapeutically effective amount of at least one compound of Formula (I) or a prodrug thereof, or a pharmaceutically acceptable salt thereof, and at least one pharmaceutically acceptable excipient. One or more pharmaceutically acceptable excipient is selected, in accordance with the pharmaceutical form and method of administration desired, from customary excipients. For examples of such excipients, see Remington's Pharmaceutical Sciences (20th ed., Mack Publishing Co. 2000).

In certain aspects, a pharmaceutical composition described herein is formulated for administration to a patient in need of such composition. Pharmaceutical compositions described herein may be administered orally, topically, transdermally, by inhalation, nasally, or buccally, or parenterally. In some embodiments, the pharmaceutical composition is administered orally. Appropriate unit administration forms include oral forms such as tablets, soft or hard capsules, powders, granules, and oral solutions or suspensions; forms for inhalative administration such as an inhaler spray; and forms for topical administration, such as creams, gels, ointments, and lotions.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the pharmaceutical composition.

In one aspect, provided herein are methods of treating a disease or condition in a subject in need thereof. In some embodiments, the method comprises administering to the subject in need thereof a therapeutically effective amount of a compound of Formula (I), or a pharmaceutically acceptable salt thereof. In some embodiments, the method comprises administering to the subject in need thereof a therapeutically effective amount of a parent drug described herein. In some embodiments, the method comprises administering to the subject in need thereof a therapeutically effective amount of an intermediate metabolite described herein. In some embodiments, the method comprises administering to the subject in need thereof a therapeutically effective amount of a prodrug described herein. In some embodiments, the disease or condition is responsive to the modulation (e.g., inhibition) of STAT6.

In one aspect, provided is a method of administering a parent drug compound of Formula (I), or a pharmaceutically acceptable salt thereof, to a subject, wherein the method comprises administering to the subject a prodrug, or a pharmaceutically acceptable salt thereof, of the parent drug compound of Formula (I), or a pharmaceutically acceptable salt thereof. In some embodiments, the prodrug once administered to the subject is metabolized in the subject to the parent drug compound of Formula (I). In some embodiments, the prodrug once administered to the subject is first metabolized to an intermediate metabolite and subsequently to the parent drug compound of Formula (I). In some embodiments, the parent drug compound of Formula (I) is a compound selected from the groups consisting of the compounds in Table 1A, or a pharmaceutically acceptable salt thereof. In some embodiments, the prodrug is a compound of Formula (I), or a pharmaceutically acceptable salt thereof, e.g., a compound selected from the group consisting of the compounds in Table 1C, or a pharmaceutically acceptable salt thereof. In some embodiments, the intermediate metabolite is a compound of Formula (I), or a pharmaceutically acceptable salt thereof, e.g., a compound selected from the group consisting of the compounds in Table 1B, or a pharmaceutically acceptable salt thereof.

In one aspect, provided is a method of administering an intermediate metabolite compound of Formula (I), or a pharmaceutically acceptable salt thereof, to a subject, wherein the method comprises administering to the subject a prodrug, or a pharmaceutically acceptable salt thereof, of the intermediate metabolite compound of Formula (I), or a pharmaceutically acceptable salt thereof. In some embodiments, the prodrug once administered to a subject is metabolized in the subject to the intermediate metabolite of Formula (I). In some embodiments the intermediate metabolite compound of Formula (I) is a compound selected from the groups consisting of the compounds in Table 1B, or a pharmaceutically acceptable salt thereof. In some embodiments the prodrug is a compound of Formula (I), or a pharmaceutically acceptable salt thereof, e.g., a compound selected from the group consisting of the compounds in Table 1C, or a pharmaceutically acceptable salt thereof.

In some embodiments, a compound of Formula (I) is selective for inhibition of STAT6 vs. STAT1, STAT2, STAT3, STAT4, STAT5a, and/or STAT5b. In some embodiments, a compound of Formula (I) is at least about 100, 200, 300, 400, 500, 600, 700, 800, 900, 1000, 1250, 1500, 1750, 2000, 2500, 3000, 3500, 4000, 4500, 5000, 6000, 7000, 8000, 9000, or 10000 times selective for STAT6 vs. STAT1, STAT3, and/or STAT4. In some embodiments, a compound of Formula (I) is at least about 100, 200, 300, 400, 500, 600, 700, 800, 900, 1000, 1250, 1500, 1750, 2000, 2500, 3000, 3500, 4000, 4500, 5000, 6000, 7000, 8000, 9000, or 10000 times selective for STAT6 vs. STAT1, STAT2, STAT3, and/or STAT4. In some embodiments, a compound of Formula (I) is at least about 100, 200, 300, 400, 500, 600, 700, 800, 900, 1000, 1250, 1500, 1750, 2000, 2500, 3000, 3500, 4000, 4500, 5000, 6000, 7000, 8000, 9000, or 10000 times selective for STAT6 vs. STAT1, STAT2, STAT3, STAT4, STAT5a, and/or STAT5b.

In some embodiments, a compound of Formula (I) inhibits IL-4 and/or IL-13 induced expression of thymus- and activation-regulated chemokine (TARC) (also known as, CC chemokine ligand 17 (CCL17)). In some embodiments, a compound of Formula (I) inhibits IL-4 and/or IL-13 induced expression of TARC in human peripheral blood mononuclear cells (PBMCs). In some embodiments, the IC₅₀ for inhibition of IL-4 and/or IL-13 induced TARC expression (e.g., in human PBMCs) is less than or equal to about 500 nM, about 100 nM, about 50 nM, about 10 nM, about 5 nM, or about 1 nM.

In some embodiments, a compound of Formula (I) inhibits IL-4 induced expression of CD23 (also known as, Fc epsilon RII (FcεRII)). In some embodiments, a compound of Formula (I) inhibits IL-4 induced expression of CD23 in human PBMCs. In some embodiments, the IC50 for inhibition of IL-4 induced CD23 expression (e.g., in human PBMCs) is less than or equal to about 500 nM, about 100 nM, about 50 nM, about 10 nM, about 5 nM, or about 1 nM.

In some embodiments, a compound of Formula (I) inhibits T helper 2 (Th2) cell function. In some embodiments, the IC₅₀ for inhibition of Th2 cell function is less than or equal to about 500 nM, about 100 nM, about 50 nM, about 10 nM, about 5 nM, or about 1 nM. In some embodiments, inhibition of Th2 cell function is measured based on change in expression of IL-5. In some embodiments, a compound of Formula (I) inhibits T helper 2 (Th2) cell function selectively compared to inhibition of other T cell functions, for example, T cell activation, Th helper 1 (Th1) cell function, and/or T helper 17 (Th17) cell function. In some embodiments, the inhibition of Th2 cell function relative to inhibition of T cell activation is greater than or equal to about 30×, about 50×, about 100×, about 300×, about 500×, or about 1000×. In some embodiments, inhibition of T cell activation is measured based on change in expression of CD25. In some embodiments, the inhibition of Th2 cell function relative to inhibition of Th1 cell function is greater than or equal to about 30×, about 50×, about 100×, about 300×, about 500×, or about 1000×. In some embodiments, inhibition of Th1 cell function is measured based on change in expression of IFNγ. In some embodiments, the inhibition of Th2 cell function relative to inhibition of Th17 cell function is greater than or equal to about 30×, about 50×, about 100×, about 300×, about 500×, or about 1000×. In some embodiments, inhibition of Th1 cell function is measured based on change in expression of IL-17A. In some embodiments, a compound of Formula (I) inhibits T helper 2 (Th2) cell function selectively compared to modulation of hematologic homeostasis, for example inhibition of erythropoietin(EPO)-induced STAT5-driven transcription and/or thrombopoietin(TPO)-induced STAT5-driven transcription. In some embodiments, the inhibition of Th2 cell function relative to inhibition of EPO induced STAT5-driven transcription is greater than or equal to about 30×, about 50×, about 100×, about 300×, about 500×, or about 1000×. In some embodiments, the inhibition of Th2 cell function relative to inhibition of TPO induced STAT5-driven transcription is greater than or equal to about 30×, about 50×, about 100×, about 300×, about 500×, or about 1000×.

In some embodiments, oral administration (e.g., P.O. in dogs) of a prodrug compound of Formula (I) provides durable bioavailability of the corresponding parent drug compound of Formula (I). In some embodiments, the oral administration (e.g., P.O. in dogs) of the prodrug compound of Formula (I) enables durable bioavailability of the parent drug in PBMCs. In some embodiments, the bioavailability is characterized by an effective concentration (e.g., in PBMCs) of the parent drug about 2 hours, about 6 hours, about 12 hours, or about 24 hours after dosing of the prodrug. In some embodiments, the bioavailability is characterized by an effective concentration (e.g., in PBMCs) of the parent drug averaged over about 6 hours, about 12 hours, or about 24 hours after dosing of the prodrug. In some embodiments the effective concentration (e.g., in PBMCs) is at least about 50 ng/mL, at least about 100 ng/mL, at least about 200 ng/mL, at least about 300 ng/mL, at least about 400 ng/mL, at least about 500 ng/mL, at least about 600 ng/mL, at least about 700 ng/mL, at least about 800 ng/mL, at least about 900 ng/mL, or at least about 1000 ng/mL. In some embodiments, the bioavailability is characterized by an area under the curve (AUC) of cellular concentration (e.g., ng/mL in PBMCs) over about 2 hours, about 6 hours, about 12 hours, or about 24 hours after dosing of the prodrug. In some embodiments, the AUC over 24 hours post-dosing is at least about 1200 ng*h/mL, at least about 2400 ng*h/mL, at least about 4800 ng*h/mL, at least about 7200 ng*h/mL, at least about 9600 ng*h/mL, at least about 12000 ng*h/mL, at least about 14400 ng*h/mL, at least about 16800 ng*h/mL, at least about 19200 ng*h/mL, or at least about 21600 ng*h/mL, at least about 24000 ng*h/mL.

In some embodiments, oral administration (e.g., P.O. in dogs) of a prodrug compound of Formula (I) the method enables durable and/or selective inhibition of pSTAT6. In some embodiments, the oral administration of a prodrug compound of Formula (I) enables therapeutically effective inhibition of pSTAT6. In some embodiments, therapeutically effective inhibition of pSTAT6 is characterized by the percentage of pSTAT6-positive cells (e.g., in whole blood, PBMCs, myeloid cells, lymphocytes) following oral administration compared to predose control. In some embodiments, the maximum percentage of pSTAT6-positive cells (e.g., in whole blood, PBMCs, myeloid cells, lymphocytes) compared to predose control following initial dose response is less than about 90%, is less than about 80%, is less than about 70%, is less than about 60%, less than about 50%, less than about 40%, less than about 30%, less than about 20%, or less than about 10%. In some embodiments, a prodrug compound of Formula (I) inhibits pSTAT6 (e.g., depletes the percentage of pSTAT6-positive cells compared to predose control to less than about 90%, less than about 80%, less than about 70%, less than about 60%, less than about 50%, less than about 40%, less than about 30%, less than about 20%, or less than about 10%) with dosing twice per day (e.g., P.O., BID in dogs). In some embodiments, a prodrug compound of Formula (I) inhibits pSTAT6 (e.g., depletes the percentage of pSTAT6-positive cells compared to predose control to less than about 90%, less than about 80%, less than about 70%, less than about 60%, less than about 50%, less than about 40%, less than about 30%, less than about 20%, or less than about 10%) with dosing once per day (e.g., P.O., QD in dogs).

In one aspect, provided herein is a method of treating a disease or condition responsive to the modulation (e.g., inhibition) of STAT6 in a subject in need thereof, comprising administering to the subject a therapeutically effective amount of a compound of Formula (I) or a prodrug thereof, or a pharmaceutically acceptable salt thereof, or a pharmaceutical composition comprising a compound of Formula (I) or a prodrug thereof, or a pharmaceutically acceptable salt thereof.

Also provided is the use of a compound of Formula (I) or a prodrug thereof, or a pharmaceutically acceptable salt thereof, or a pharmaceutical composition comprising a compound of Formula (I) or a prodrug thereof, or a pharmaceutically acceptable salt thereof, for the manufacture of a medicament. Also provided is the use of a compound of Formula (I) or a prodrug thereof, or a pharmaceutically acceptable salt thereof, or a pharmaceutical composition comprising a compound of Formula (I) or a prodrug thereof, or a pharmaceutically acceptable salt thereof, for the manufacture of a medicament for treating a disease or condition responsive to the modulation (e.g., inhibition) of STAT6. Also provided is a compound of Formula (I) or a prodrug thereof, or a pharmaceutically acceptable salt thereof, or a pharmaceutical composition comprising a compound of Formula (I) or a prodrug thereof, or a pharmaceutically acceptable salt thereof, for use as a drug. Also provided is a compound of Formula (I) or a prodrug thereof, or a pharmaceutically acceptable salt thereof, or a pharmaceutical composition comprising a compound of Formula (I) or a prodrug thereof, or a pharmaceutically acceptable salt thereof, for use in treating a disease or condition responsive to the modulation (e.g., inhibition) of STAT6. In some embodiments, the compound is selected from the group consisting of the parent drugs in Table 1A, or a pharmaceutically acceptable salt thereof. In some embodiments, the compound is selected from the group consisting of the parent drugs in Table 1A, or a prodrug or intermediate metabolite thereof, or a pharmaceutically acceptable salt thereof. In some embodiments, the compound is selected from the group consisting of the prodrugs in Table 1C, or a pharmaceutically acceptable salt thereof. In some embodiments, the compound, or a pharmaceutically acceptable salt thereof, is selected from the group consisting of parent drugs A3, A4, A5, A6, A7, A8, A11, A13, A14, A18, A19, A20, A21, A22, A23, A25, A30, A32, A33, A35, A36, A37, A38, A40, A42, A43, A44, A47, A48, A49, A50, A52, A53, A55, A57, A58, A59, A60, A61, A65, and pharmaceutically acceptable salts thereof. In some embodiments, the compound, or a pharmaceutically acceptable salt thereof, is selected from the group consisting of compounds A3, A4, A5, A6, A7, A8, A11, A13, A14, A18, A19, A20, A21, A22, A23, A25, A30, A32, A33, A35, A36, A37, A38, A40, A42, A43, A44, A47, A48, A49, A50, A52, A53, A55, A57, A58, A59, A60, A61, and A65, prodrugs or intermediate metabolites thereof, and pharmaceutically acceptable salts of any of the foregoing. In some embodiments, the compound, or a pharmaceutically acceptable salt thereof, is selected from the group consisting of prodrugs C4, C5, C6, C7, C8, C10, C13, C17, C19, C23, C25, C53, C54, C61, C63, C64, C65, C66, C68, C70, C71, C73, C74, C75, C82, C83, C84, C89, C90, C91, C92, C93, C94, C96, C97, C96, C97, C102, C103, C015, C016, C017, C110, C111, C114, C116, C118, C121, C122, C123, C124, C125, C126, C127, C132, C133, C135, C136, C137, C140, C141, C143, C144, C148, C155, C156, C160, C162, C170, C173, C175, C176, C177, C180, C182, C185, C186, C188, C189, C194, C195, C197, C198, C205, C208, and C209, and pharmaceutically acceptable salts thereof.

In one aspect, the disease or condition responsive to the modulation (e.g., inhibition) of STAT6 is selected from the group consisting of an inflammatory disorder, autoimmune disorder, an allergic disorder, and a skin disorder. In some embodiments, the disease or condition is associated with T helper type 2 (Th2) inflammation. In some embodiments, the disease or condition is selected from the group consisting of atopic dermatitis, prurigo nodularis, asthma, chronic rhinosinusitis with nasal polyps, eosinophilic esophagitis, and chronic obstructive pulmonary disease.

In another aspect, provided herein is a method of modulating the activity of STAT6, comprising contacting a biological sample with or administering to a subject in need thereof a compound of Formula (I) or a prodrug thereof, or a pharmaceutically acceptable salt thereof, or a pharmaceutical composition comprising a compound of Formula (I) or a prodrug compound thereof, or a pharmaceutically acceptable salt thereof. In some embodiments, the method inhibits the activity of STAT6. In some embodiments, the method inhibits the activity of phospho-STAT6 (pSTAT6). In some embodiments, the method inhibits the activity of STAT6 and pSTAT6. In some embodiments, the method inhibits the phosphorylation of STAT6 to pSTAT6, for example by a JAK protein (e.g., JAK1). In some embodiments, the method inhibits the dimerization of pSTAT6. In some embodiments, the method inhibits the translocation of pSTAT6 into the nucleus.

### ENUMERATED EMBODIMENTS

The following enumerated embodiments are representative of some aspects of the invention.
1. A compound of Formula (I),

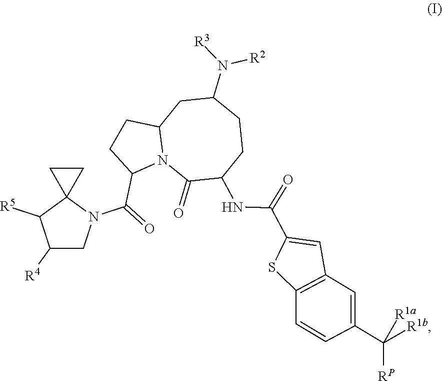

or a pharmaceutically acceptable salt thereof, wherein:
R^{1a} and R^{1b} are each independently H or halogen;
R² is H, (C₁-C₆)alkyl, or (C₃-C₆)cycloalkyl;
R³ is (C₁-C₆)alkyl, halo(C₁-C₆)alkyl, (C₃-C₆)cycloalkyl, 3- to 7-membered heterocyclyl, —(C₁-C₆)alkylene-(C₃-C₆)cycloalkyl, or —(C₁-C₆)alkylene-(3- to 7-membered heterocyclyl), wherein (C₁-C₆)alkyl, (C₃-C₆)cycloalkyl, (C₃-C₆)cycloalkyl of —(C₁-C₆)alkylene-(C₃-C₆)cycloalkyl, or (3- to 7-membered heterocyclyl) of —(C₁-C₆)alkylene-(3- to 7-membered heterocyclyl) is each substituted with 0, 1, 2, 3, 4, or 5 substituents each independently selected from the group consisting of (C₁-C₆)alkyl, (C₁-C₆)alkoxy, cyano, halogen, halo(C₁-C₆)alkyl, and halo(C₁-C₆)alkoxy,
or R² and R³ together with the nitrogen atom to which they are attached form 5- to 7-membered heterocyclyl substituted with 0, 1, 2, or 3 substituents each independently selected from the group consisting of (C₁-C₆)alkyl, (C₁-C₆)alkoxy, cyano, halogen, halo(C₁-C₆)alkyl, and halo(C₁-C₆)alkoxy;
R⁴ is H, (C₆-C₁₀)aryl, or 5- to 10-membered heteroaryl, wherein (C₆-C₁₀)aryl or 5- to 10-membered heteroaryl is each substituted with 0, 1, 2, or 3 substituents each independently selected from the group consisting of halogen, cyano, (C₁-C₆)alkyl, (C₁-C₆)alkoxy, halo(C₁-C₆)alkyl, halo(C₁-C₆)alkoxy, and —NR^{4a}R^{4b}, wherein:
   each R^{4a} and R^{4b} is independently H or (C₁-C₆)alkyl;
R⁵ is H, cyano, or (C₁-C₆)alkoxy; and
R^{P} is phosphonic acid, phosphonate, phosphonamidate, or phosphondiamidate.
2. The compound of embodiment 1, or a pharmaceutically acceptable salt thereof, wherein R^{1a} is F.
3. The compound of embodiment 1, or a pharmaceutically acceptable salt thereof, wherein R^{1b} is H.
4. The compound of embodiment 1, or a pharmaceutically acceptable salt thereof, wherein R^{1a} is F and R^{1b} is H.
5. The compound of embodiment 1, or a pharmaceutically acceptable salt thereof, wherein R^{1a} and R^{1b} are each F.
6. The compound of any one of embodiments 1 to 5, or a pharmaceutically acceptable salt thereof, wherein R² is methyl or ethyl.
7. The compound of any one of embodiments 1 to 6, or a pharmaceutically acceptable salt thereof, wherein R³ is (C₁-C₆)alkyl, halo(C₁-C₆)alkyl, (C₃-C₆)cycloalkyl, 3- to 7-membered heterocyclyl, or —(C₁-C₆)alkylene-(3- to 7-membered heterocyclyl).
8. The compound of embodiment 7, or a pharmaceutically acceptable salt thereof, wherein R³ is halo(C₁-C₆)alkyl.
9. The compound of embodiment 7, or a pharmaceutically acceptable salt thereof, wherein R³ is (C₃-C₆)cycloalkyl substituted with 0, 1, 2, or 3 substituents each independently selected from the group consisting of halogen, cyano, halo(C₁-C₆)alkyl, and halo(C₁-C₆)alkoxy.
10. The compound of embodiment 7, or a pharmaceutically acceptable salt thereof, wherein R³ is 3- to 7-membered heterocyclyl.
11. The compound of embodiment 7, or a pharmaceutically acceptable salt thereof, wherein R³ is —(C₁-C₆)alkylene-[3- to 7-membered heterocyclyl substituted with 0 or 1 (C₁-C₆)alkyl].
12. The compound of embodiment 7, or a pharmaceutically acceptable salt thereof, wherein R³ is (C₁-C₆)alkyl substituted with 0, 1, 2, 3, 4, or 5 substituents each independently selected from the group consisting of halo, (C₁-C₆)alkoxy, and halo(C₁-C₆)alkoxy.
13. The compound of embodiment 7, or a pharmaceutically acceptable salt thereof, wherein R³ is

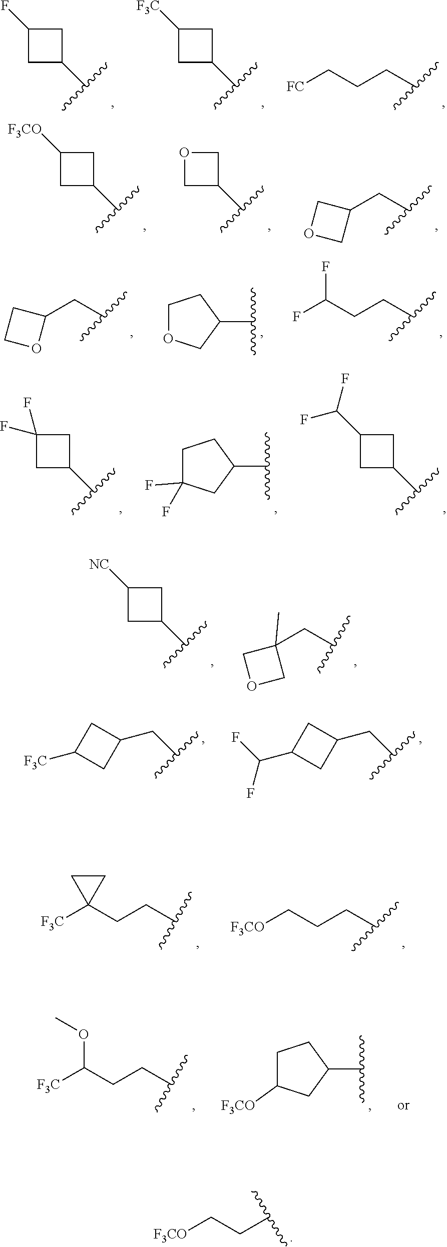

14. The compound of embodiment 7, or a pharmaceutically acceptable salt thereof, wherein

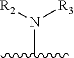

of Formula (I) is

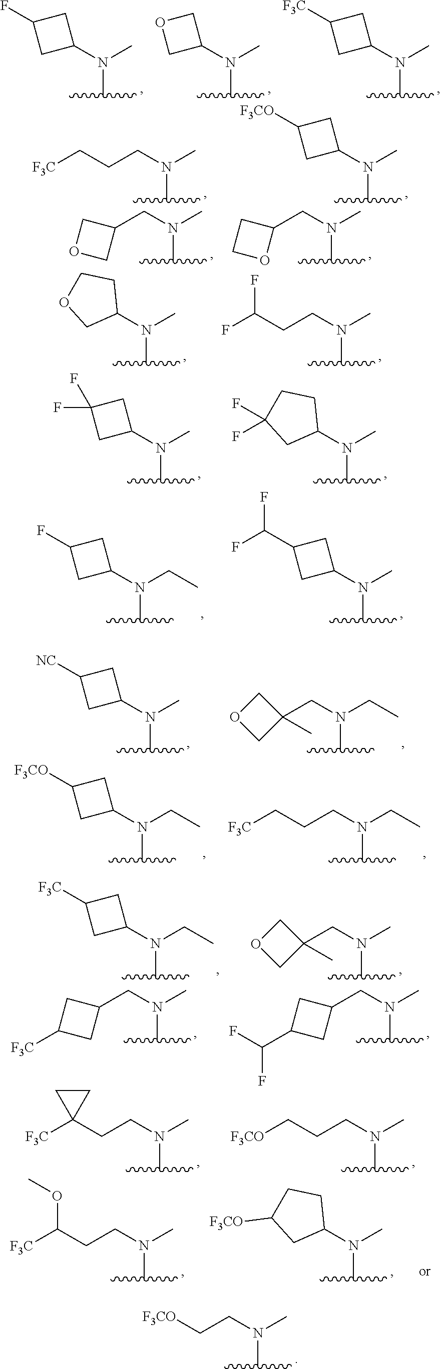

15. The compound of any one of embodiments 1 to 5, or a pharmaceutically acceptable salt thereof, wherein R² and R³ together with the nitrogen atom to which they are attached form 6-membered heterocyclyl.
16. The compound of embodiment 15, or a pharmaceutically acceptable salt thereof, wherein

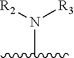

of Formula (I) is

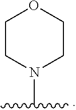 [SMILES: *N1CCOCC1]

17. The compound of any one of embodiments 1 to 16, or a pharmaceutically acceptable salt thereof, wherein R⁴ is H.
18. The compound of any one of embodiments 1 to 16, or a pharmaceutically acceptable salt thereof, wherein R⁴ is (C₆-C₁₀)aryl or 5- to 10-membered heteroaryl.
19. The compound of embodiment 18, or a pharmaceutically acceptable salt thereof, wherein R⁴ is phenyl substituted with 0, 1, 2, or 3 halogen.
20. The compound of embodiment 19, or a pharmaceutically acceptable salt thereof, wherein R⁴ is

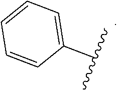 [SMILES: *c1ccccc1]

21. The compound of embodiment 19, or a pharmaceutically acceptable salt thereof, wherein R⁴ is

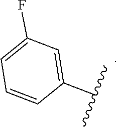 [SMILES: *c1cccc(F)c1]

22. The compound of embodiment 18, or a pharmaceutically acceptable salt thereof, wherein R⁴ is pyridinyl substituted with 0 or 1 —NR^{4a}R^{4b}.
23. The compound of embodiment 22, or a pharmaceutically acceptable salt thereof, wherein R⁴ is

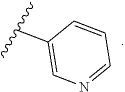 [SMILES: *c1cccnc1]

24. The compound of embodiment 22, or a pharmaceutically acceptable salt thereof, wherein R⁴ is

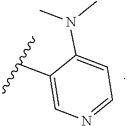 [SMILES: *c1cnccc1N(C)C]

25. The compound of any one of embodiments 1 to 24, or a pharmaceutically acceptable salt thereof, wherein R⁵ is H.
26. The compound of any one of embodiments 1 to 24, or a pharmaceutically acceptable salt thereof, wherein R⁵ is cyano.
27. The compound of any one of embodiments 1 to 24, or a pharmaceutically acceptable salt thereof, wherein R⁵ is methoxy.
28. The compound of any one of embodiments 1 to 27, or a pharmaceutically acceptable salt thereof, wherein R^{P} is

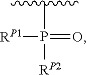

wherein R^{P1} and R^{P2} are each independently —OH, —OR^{Pa}, N-linked amino acid, N-linked amino acid ester, or

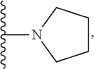 [SMILES: *N1CCCC1]

wherein

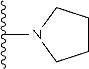 [SMILES: *N1CCCC1]

is substituted with 0, 1, 2, or 3 substituents each independently selected from the group consisting of halo, oxo, COOH, and —C(O)OR^{PE}, wherein:
each R^{Pa} is independently (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy; and
each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.
29. The compound of any one of embodiments 1 to 27, or a pharmaceutically acceptable salt thereof, wherein R^{P} is

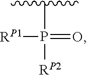

wherein R^{P1} and R^{P2} are each independently —OH, —OR^{Pa}, —NR^{Paa1}CR^{Paa2}R^{Paa3}C(O)OH, —NR^{Paa1}CR^{Paa2}R^{Paa3}C(O)OR^{Paa4}, or

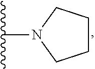 [SMILES: *N1CCCC1]

wherein

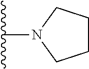 [SMILES: *N1CCCC1]

is substituted with 0, 1, 2, or 3 substituents each independently selected from the group consisting of halo, oxo, COOH, and —C(O)OR^{PE}, wherein:
each R^{Pa} is independently (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy; each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen;
each R^{Paa1} is independently H or (C₁-C₆)alkyl;
each R^{Paa2} is independently H or (C₁-C₆)alkyl, and each R^{Paa3} is independently H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3}, together with the carbon atom to which they are attached form (C₃-C₆)cycloalkyl; and
each R^{Paa4} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.
30. The compound of any one of embodiments 1 to 27, or a pharmaceutically acceptable salt thereof, wherein R^{P} is

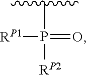

wherein R^{P1} and R^{P2} are each —OH.
31. The compound of any one of embodiments 1 to 27, or a pharmaceutically acceptable salt thereof, wherein R^{P} is

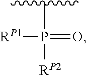

wherein R^{P1} is —OH and R^{Paa2} is N-linked amino acid.
32. The compound of any one of embodiments 1 to 27, or a pharmaceutically acceptable salt thereof, wherein R^{P} is

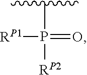

wherein R^{P1} is —OR^{P}a and R^{Paa2} is N-linked amino acid ester, wherein R^{Pa} is (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy.
33. The compound of any one of embodiments 1 to 27, or a pharmaceutically acceptable salt thereof, wherein R^{P} is

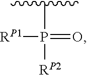

wherein R^{P1} and R^{Paa2} are each independently N-linked amino acid.
34. The compound of any one of embodiments 1 to 27, or a pharmaceutically acceptable salt thereof, wherein R^{P} is

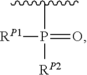

wherein R^{P1} and R^{P2} are each independently N-linked amino acid ester.
35. The compound of any one of embodiments 1 to 27, or a pharmaceutically acceptable salt thereof, wherein R^{P} is

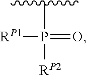

R^{P2}, wherein R^{P1} is N-linked amino acid and R^{P2} is

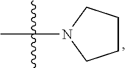 [SMILES: *N1CCCC1]

wherein

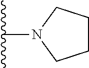 [SMILES: *N1CCCC1]

is substituted with 0, 1, 2, or 3 substituents each independently selected from the group consisting of halo, oxo, and —COOH.
36. The compound of any one of embodiments 1 to 27, or a pharmaceutically acceptable salt thereof, wherein R^{P} is

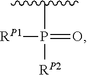

wherein R^{P1} is N-linked amino acid ester and R^{P2} is

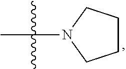 [SMILES: *N1CCCC1]

wherein

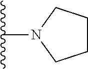 [SMILES: *N1CCCC1]

is substituted with 0, 1, 2, or 3 substituents each independently selected from the group consisting of halo, oxo, and ester.
37. The compound of embodiment 32, or a pharmaceutically acceptable salt thereof, wherein R^{Pa} is phenyl.
38. The compound of embodiment 32, or a pharmaceutically acceptable salt thereof, wherein R^{Pa} is ethyl.
39. The compound of embodiment 32, or a pharmaceutically acceptable salt thereof, wherein R^{Pa} is —CH₂CF₃.
40. The compound of embodiment 32, or a pharmaceutically acceptable salt thereof, wherein R^{Pa} is

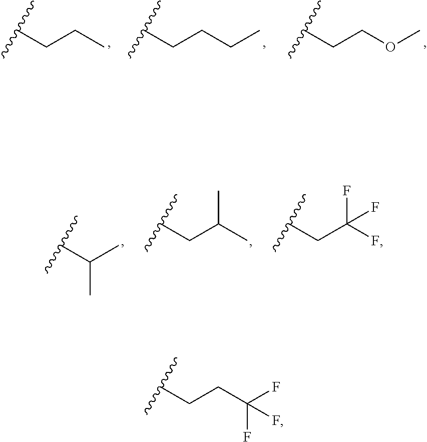

cyclopropyl, cyclopentyl, or cyclohexyl.
41. The compound of embodiment 35, or a pharmaceutically acceptable salt thereof, wherein R² is

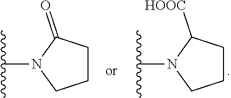 [SMILES: *N1CCCC1=O ; *N1CCCC1C(=O)O]

42. The compound of embodiment 36, or a pharmaceutically acceptable salt thereof, wherein R^{P2} is

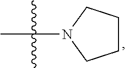 [SMILES: *N1CCCC1]

wherein

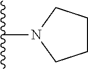 [SMILES: *N1CCCC1]

is substituted with 0, 1, 2, or 3 substituents each independently selected from the group consisting of halo, oxo, and —C(O)OR^{PE}, wherein each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.
43. The compound of embodiment 42, or a pharmaceutically acceptable salt thereof, wherein each R^{PE} is independently ethyl,

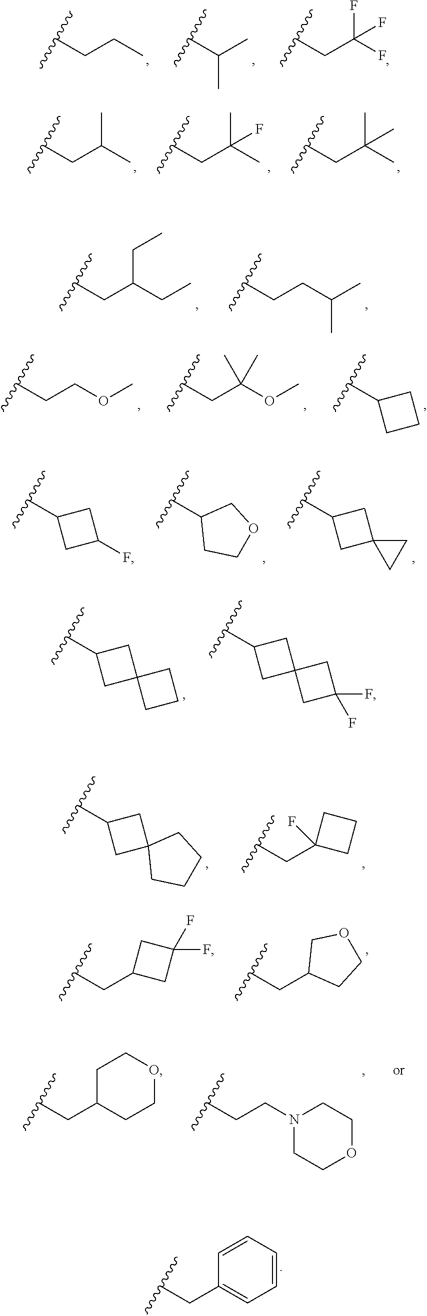

44. The compound of embodiment 43, or a pharmaceutically acceptable salt thereof, wherein each R^{PE} is

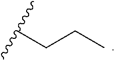 [SMILES: *CCC]

45. The compound of embodiment 36, or a pharmaceutically acceptable salt thereof, wherein R² is

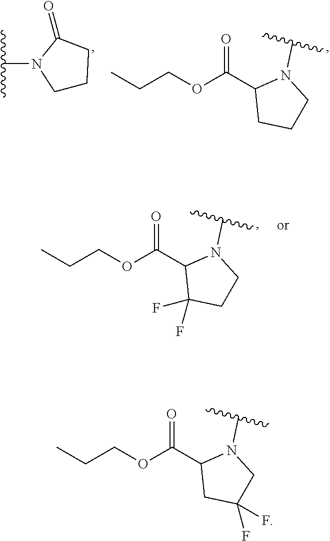

46. The compound of any one of embodiments 31, 33, 35, and 41, or a pharmaceutically acceptable salt thereof, wherein each N-linked amino acid is independently N-linked α-amino acid.
47. The compound of embodiment 46, or a pharmaceutically acceptable salt thereof, wherein each N-linked amino acid is independently —NR^{Paa1}CR^{Paa2}R^{Paa3}C(O)OH, wherein:
   each R^{Paa1} is independently H or (C₁-C₆)alkyl; and
   each R^{Paa2} is independently H or (C₁-C₆)alkyl, and each R^{Paa3} is independently H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3}, together with the carbon atom to which they are attached form (C₃-C₆)cycloalkyl.
48. The compound of any one of embodiments 32, 34, 36, 37 to 40, and 42 to 45, or a pharmaceutically acceptable salt thereof, wherein each N-linked amino acid ester is independently N-linked α-amino acid ester.
49. The compound of embodiment 48, or a pharmaceutically acceptable salt thereof, wherein each N-linked amino acid ester is independently —NR^{Paa1}CR^{Paa2}R^{Paa3}C(O)OR^{Paa4}, wherein:
   each R^{Paa1} is independently H or (C₁-C₆)alkyl;
   each R^{Paa2} is independently H or (C₁-C₆)alkyl, and each R^{Paa3} is independently H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3}, together with the carbon atom to which they are attached form (C₃-C₆)cycloalkyl; and
   each R^{Paa4} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.
50. The compound of embodiment 49, or a pharmaceutically acceptable salt thereof, wherein each R^{Paa4} is independently ethyl,

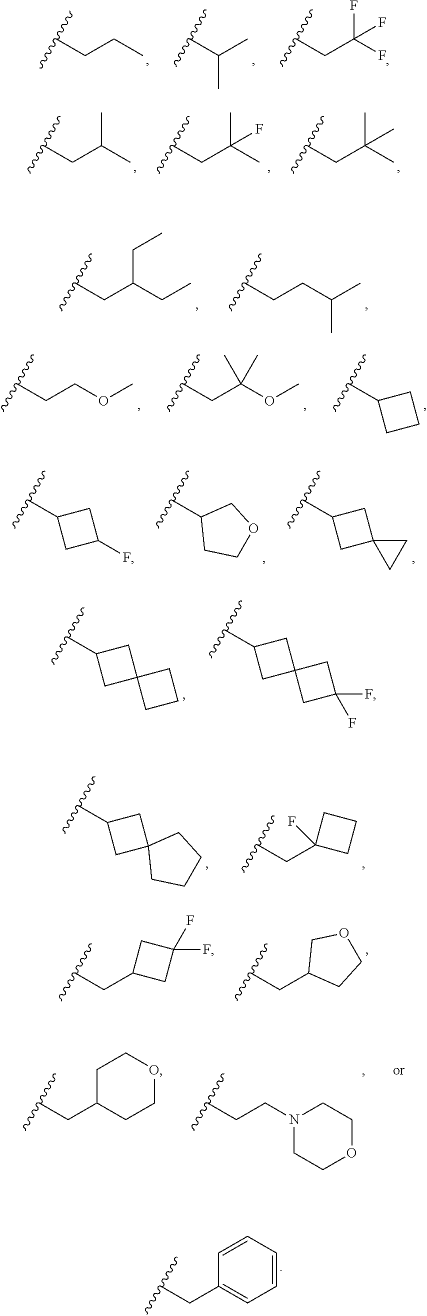

51. The compound of embodiment 50, or a pharmaceutically acceptable salt thereof, wherein R^{Paa4} is ethyl

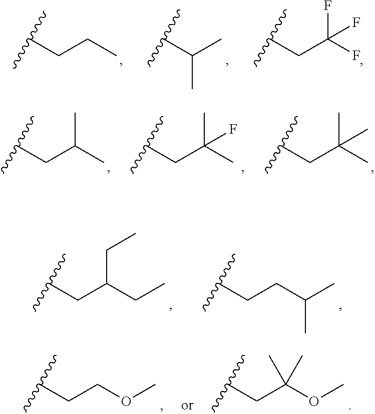

52. The compound of embodiment 50, or a pharmaceutically acceptable salt thereof, wherein R^{Paa4} is

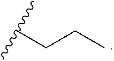 [SMILES: *CCC]

53. The compound of Embodiment 50, or a pharmaceutically acceptable salt thereof, wherein R^{Paa4} is

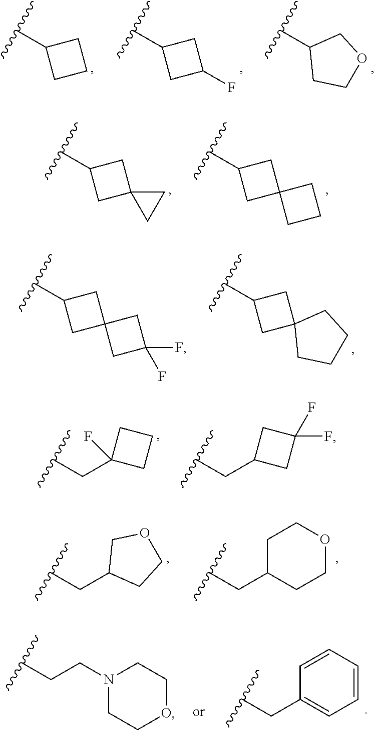

54. The compound of any one of embodiments 47 and 49 to 53, or a pharmaceutically acceptable salt thereof, wherein R^{Paa1} is H.
55. The compound of any one of embodiments 47 and 49 to 53, or a pharmaceutically acceptable salt thereof, wherein R^{Paa1} is methyl.
56. The compound of any one of embodiments 47 and 49 to 55, or a pharmaceutically acceptable salt thereof, wherein R^{Paa2} is H.
57. The compound of any one of embodiments 47 and 49 to 55, or a pharmaceutically acceptable salt thereof, wherein R^{Paa2} is methyl.
58. The compound of any one of embodiments 47 and 49 to 57, or a pharmaceutically acceptable salt thereof, wherein R^{Paa3} is methyl.
59. The compound of any one of embodiments 47 and 49 to 57, or a pharmaceutically acceptable salt thereof, wherein R^{Paa3} is ethyl,

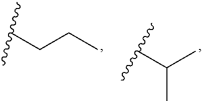

or —CH₂OCH₃.
60. The compound of any one of embodiments 47 and 49 to 57, or a pharmaceutically acceptable salt thereof, wherein R^{Paa3} is

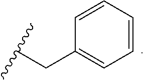 [SMILES: *Cc1ccccc1]

61. The compound of any one of embodiments 47 and 49 to 53 or a pharmaceutically acceptable salt thereof, wherein R^{Paa2} and R^{Paa3}, together with the carbon atom to which they are attached form cyclopropyl.
62. The compound of embodiment 1, or a pharmaceutically acceptable salt thereof, wherein the compound is selected from the group consisting of the compounds of Table 1A, Table 1B, and Table 1C.
63. A pharmaceutical composition comprising the compound of any one of embodiments 1 to 62, or a pharmaceutically acceptable salt thereof; and a pharmaceutically acceptable excipient.
64. A medicament comprising a compound of any one of embodiments 1 to 62, or a pharmaceutically acceptable salt thereof, or the pharmaceutical composition of embodiment 63.
65. A method of treating a disease or condition responsive to the modulation (e.g., inhibition) of STAT6 in a subject in need thereof, the method comprising administering to the subject in need thereof a therapeutically effective amount of the compound of any one of embodiments 1 to 62, or a pharmaceutically acceptable salt thereof, or the pharmaceutical composition of embodiment 63.
66. A compound of any one of embodiments 1 to 62, or a pharmaceutically acceptable salt thereof, or the pharmaceutical composition of embodiment 63, for use as a drug.
67. A compound of any one of embodiments 1 to 62, or a pharmaceutically acceptable salt thereof, or the pharmaceutical composition of embodiment 63, for use in the treatment of a disease or condition responsive to the modulation (e.g., inhibition) of STAT6 in a subject.
68. Use of a compound of any one of embodiments 1 to 62, or a pharmaceutically acceptable salt thereof, or the pharmaceutical composition of embodiment 63, for use in the preparation of a medicament.
69. The use of embodiment 67, wherein the medicament is intended for the treatment of a disease or condition responsive to the modulation (e.g., inhibition) of STAT6 in a subject.
70. The method of embodiment 65, the compound for use of embodiment 67, or the use of embodiment 69, wherein the disease or condition is selected from the group consisting of an inflammatory disorder, autoimmune disorder, an allergic disorder, and a skin disorder.
71. The method of embodiment 65, the compound for use of embodiment 67, or the use of embodiment 69, wherein the disease or condition is selected from the group consisting of atopic dermatitis, prurigo nodularis, asthma, chronic rhinosinusitis with nasal polyps, eosinophilic esophagitis, and chronic obstructive pulmonary disease.

### EXAMPLES

The presently disclosed subject matter will be better understood by reference to the following Examples, which are provided as exemplary of the invention, and not by way of limitation.

Abbreviations used in the Examples include the following:
ACN, MeCN—acetonitrile
AIBN—Azobisisobutyronitrile
AcOH—acetic acid
BSA—Bovine serum albumin
CMPI—2-chloro-1-methylpyridinium iodide
CST—cell signaling technology
DCM—dichloromethane
DEA—diethanoloamine
DFT—density functional theory
DIEA—N,N-diisopropylethylamine
DMAP—4-Dimethylaminopyridine
DME—dimethyl ether
DMF—dimethylformamide
DMSO—dimethyl sulfoxide
DTT—Dithiothreitol
EA, EtOAc—ethyl acetate
EDCI—1-Ethyl-3-(3-dimethylaminopropyl)carbodiimide hydrochloride
EDTA—Ethylenediaminetetraacetic acid
ESI—electrospray ionization
FA—formic acid
FBS—fetal bovine serum
HATU—1-[Bis(dimethylamino)methylene]-1H-1,2,3-triazolo[4,5-b]pyridinium 3-oxide hexafluorophosphate (Hexafluorophosphate Azabenzotriazole Tetramethyl Uronium)
HEPES—4-(2-Hydroxyethyl)piperazine-1-ethane-sulfonic acid
HPLC—high-performance liquid chromatography
IMDM—Iscove's Modified Dulbecco's Medium
IPA—isopropyl alcohol
LCMS—liquid chromatography-mass spectrometry
LDA—Lithium diisopropylamide
MeOH—methyl alcohol
MS—mass spectrometry
MSD—meso scale discovery
NBS—N-Bromosuccinimide
NMR—nuclear magnetic resonance_
PBS—phosphate buffered saline
PE—phycoerythrin
PK—pharmacokinetic
PMBC—peripheral blood mononuclear cells
qPCR—quantitative polymerase chain reaction
RBC—red blood cell
RT—retention time
SFC—supercritical fluid chromatography
SM—starting material
TEA—triethylamine
TFA—trifluoroacetic acid
THF—tetrahydrofuran
TLC—thin layer chromatography
TMSBr—Bromotrimethylsilane
TMSI—Trimethylsilyl iodide
VCD—vibrational circular dichroism
aq—aqueous
eq—equivalents
Hz—hertz
MHz—megahertz
h, hr—hour
g—gram
mg—milligram
L—liter
mL—milliliter
uL—microliter
m/z—mass-to-charge ratio
mm—millimeter
nm—nanometer
um—micrometer
min—minute
mol—mole
mmol—millimole
umol—micromole
M—molarity
mM—millimolar
N—normality

### Example 1: Preparation of Compounds

The compounds of Formula (I) were prepared by adapting or following the procedures outlined in the following schemes and examples. For example, the representative procedures for the combination of cores, pyrrolidine groups, northern amines, and phosphoryl groups are not limited to the examples below and may be adapted to form other compounds of Formula (I) by utilizing the appropriate intermediates and/or starting materials (e.g., pyrrolidine groups, northern amines, and/or phosphoryl groups). In some embodiments, the compounds of Formula (I) or starting materials or intermediates thereto were prepared by adapting or following procedures described in WO 2023/133336, paragraphs [0085]-[001573](EXEMPLIFICATION: Preparation of Compounds), WO 2023/164680 paragraphs [0019]-[00801](EXEMPLIFICATION: Preparation of Compounds), or WO 2023/192960, paragraphs [0085]-[00784](EXEMPLIFICATION: Preparation of Compounds), each of which is incorporated herein by reference in its entirety.

Compound names were generated using the software built into ChemDraw. To the extent that there are discrepancies between the name of a compound and its depicted structure, the depicted chemical structure is to be taken as the appropriate compound.

### Representative Procedures for Combination of Cores, Pyrrolidine Groups, Northern Amines, and Phosphoryl Groups:

The compounds of Formula (I) in Tables 1A, 1B, and 1C were prepared according to the general schemes and exemplary procedures for the combination of cores, pyrrolidine groups, northern amines, and phosphoryl groups with the appropriate starting materials, intermediates, and modifications.

### Preparation of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (Int 1)

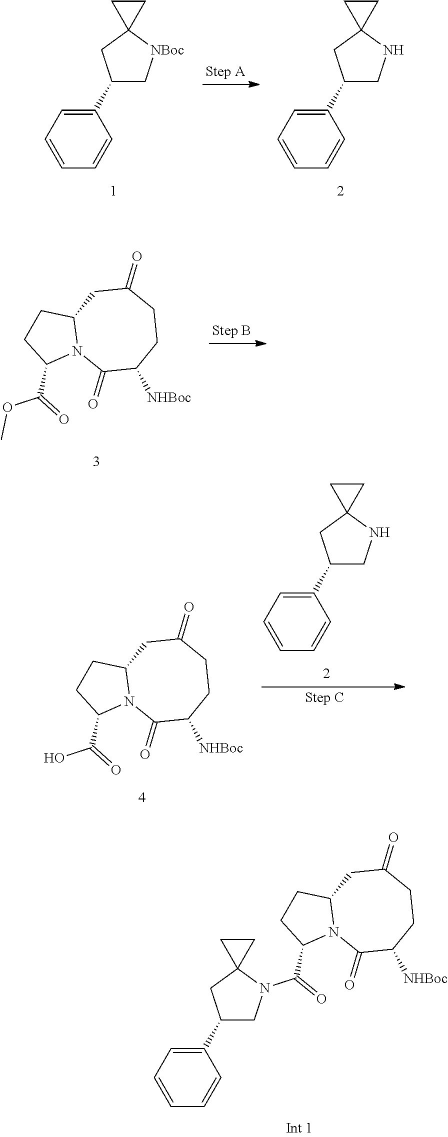 [SMILES: CC(C)(C)OC(=O)N1C[C@H](c2ccccc2)CC12CC2 ; c1ccc([C@H]2CNC3(CC3)C2)cc1 ; COC(=O)[C@@H]1CC[C@@H]2CC(=O)CC[C@H](NC(=O)OC(C)(C)C)C(=O)N21 ; CC(C)(C)OC(=O)N[C@H]1CCC(=O)C[C@H]2CC[C@@H](C(=O)O)N2C1=O ; c1ccc([C@H]2CNC3(CC3)C2)cc1 ; CC(C)(C)OC(=O)N[C@H]1CCC(=O)C[C@H]2CC[C@@H](C(=O)N3C[C@H](c4ccccc4)CC34CC4)N2C1=O]

### Step A: (S)-6-phenyl-4-azaspiro[2.4]heptane

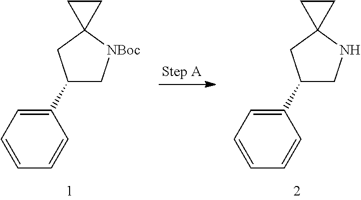 [SMILES: CC(C)(C)OC(=O)N1C[C@H](c2ccccc2)CC12CC2 ; c1ccc([C@H]2CNC3(CC3)C2)cc1]

To a solution of tert-butyl (S)-6-phenyl-4-azaspiro[2.4]heptane-4-carboxylate (1, 20 g, 73.3 mmol, 1.0 eq.) in DCM (210 mL) was added trifluoroacetic acid (TFA) (70 mL), and the resulting mixture was stirred at room temperature for 2 hrs. After completion, the reaction mixture concentrated under reduced pressure to give crude (S)-6-phenyl-4-azaspiro[2.4]heptane (TFA salt) (2, 20 g, quant.) as a white solid, which was used in next Step directly without further purification. LCMS (ESI): m/z=174 [M+H]⁺.

### Step B: (3S,6S,10aR)-6-((tert-butoxycarbonyl)amino)-5,9-dioxodecahydropyrrolo[1,2-a]azocine-3-carboxylic acid

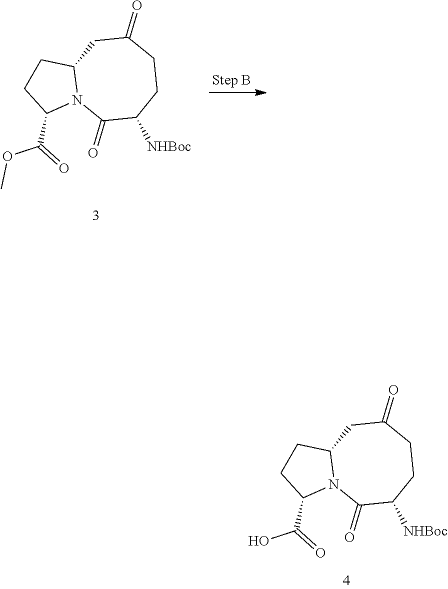 [SMILES: COC(=O)[C@@H]1CC[C@@H]2CC(=O)CC[C@H](NC(=O)OC(C)(C)C)C(=O)N21 ; CC(C)(C)OC(=O)N[C@H]1CCC(=O)C[C@H]2CC[C@@H](C(=O)O)N2C1=O]

To a solution of methyl (3S,6S,10aR)-6-((tert-butoxycarbonyl)amino)-5,9-dioxodecahydropyrrolo[1,2-a]azocine-3-carboxylate (3, 30 g, 84.7 mmol, 1.0 eq.) in THF/H₂O (300 mL/60 mL) was added LiOH—H₂O (10.4 g, 254 mmol, 3.0 eq.), and the resulting mixture was stirred at room temperature for 2 hrs. After completion, the reaction mixture was cooled down in an ice bath, then neutralized carefully with HCl (aq., 1 N) until the pH was adjusted to pH=5-6. The resulting mixture was extracted with DCM (300 mL×3), and the combined organic layers were washed with brine (100 mL×2), dried over with anhydrous Na₂SO₄, then concentrated under reduced pressure to give crude (3S,6S,10aR)-6-((tert-butoxycarbonyl)amino)-5,9-dioxodecahydropyrrolo[1,2-a]azocine-3-carboxylic acid (4, 26 g, quant.) as a white solid, which was used in next Step directly without further purification. LCMS (ESI): m/z=341 [M+H]⁺.

### Step C: tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate

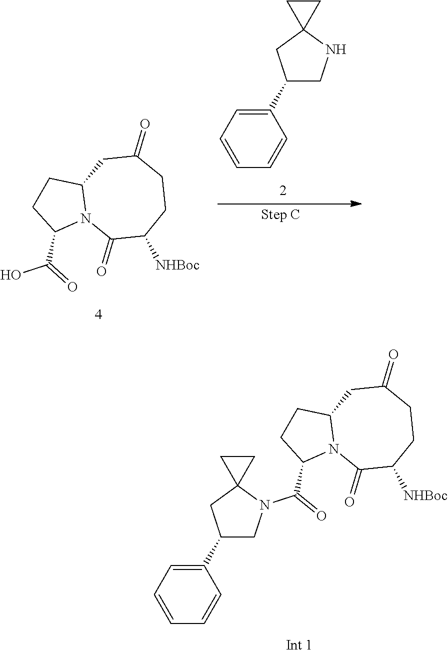 [SMILES: CC(C)(C)OC(=O)N[C@H]1CCC(=O)C[C@H]2CC[C@@H](C(=O)O)N2C1=O ; c1ccc([C@H]2CNC3(CC3)C2)cc1 ; CC(C)(C)OC(=O)N[C@H]1CCC(=O)C[C@H]2CC[C@@H](C(=O)N3C[C@H](c4ccccc4)CC34CC4)N2C1=O]

A solution of (3S,6S,10aR)-6-((tert-butoxycarbonyl)amino)-5,9-dioxodecahydropyrrolo[1,2-a]azocine-3-carboxylic acid (4, 26 g, 76.5 mmol, 1.0 eq.) and HATU (32 g, 84.2 mmol, 1.1 eq.) in DMF (500 mL) was stirred for 15 min, then TEA (38.6 g, 383 mmol, 5.0 eq.) and (S)-6-phenyl-4-azaspiro[2.4]heptane (TFA salt) (2, 13.3 g, 76.5 mmol, 1.0 eq.) were added, and the resulting mixture was stirred at room temperature for additional 2 hrs. After completion, the reaction was poured into H₂O (1 L), and the suspension was filtered. The wet cake was slurried with H₂O (1 L) and then filtered. The filter cake was washed with H₂O (100 mL×3). The wet cake was dried to afford tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (Int 1, 34 g, 68.6 mmol, 90%) as a white solid. LCMS (ESI): m/z=496 [M+H]⁺.

### Preparation of tert-butyl ((3S,6S,9S,10aR)-9-morpholino-5-oxo-3-((S)-6-phenyl-4-azaspiro[2.4]heptane-4-carbonyl)decahydropyrrolo[1,2-a]azocin-6-yl)carbamate

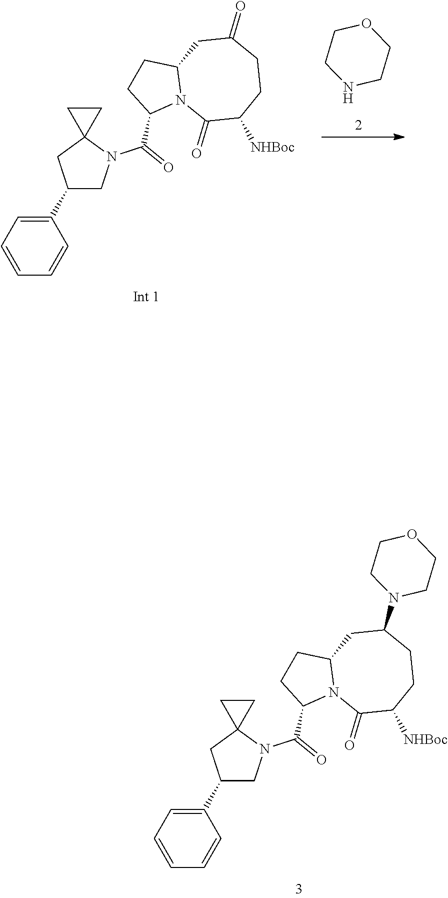 [SMILES: CC(C)(C)OC(=O)N[C@H]1CCC(=O)C[C@H]2CC[C@@H](C(=O)N3C[C@H](c4ccccc4)CC34CC4)N2C1=O ; C1COCCN1 ; CC(C)(C)OC(=O)N[C@H]1CC[C@H](N2CCOCC2)C[C@H]2CC[C@@H](C(=O)N3C[C@H](c4ccccc4)CC34CC4)N2C1=O]

To a mixture of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (Int 1, 3 g, 6.06 mmol, 1.0 eq.) and morpholine (2, 2.64 g, 30.3 mmol, 5 eq.) in MeOH (100 mL) was added ZnCl₂ (8.12 g, 60.6 mmol, 10 eq.). The resulting mixture was stirred at 55° C. for 1 hr under N₂, then NaBH₃CN (1.27 g, 20.2 mmol, 10.0 eq.) was added to the reaction mixture in five portions (2 eq/hour) and the resulting mixture was stirred at 55° C. overnight under N₂. After completion, the reaction mixture was concentrated under reduced pressure to remove MeOH (15 mL left). The residue was poured into H₂O (150 mL) and then the suspension was filtered. The wet cake was dissolved in DCM (100 mL), dried over anhydrous Na₂SO₄ and concentrated under reduced pressure. The filtrate above was then extracted with DCM (50 mL×3). The organic layers were combined and washed with brine (100 mL), dried over anhydrous Na₂SO₄, and concentrated under reduced pressure. The residue was purified by reverse phase chromatography using a 120 g C18 cartridge eluting with a gradient of 5-80% MeCN in water (with 0.1% NH₄OH) to afford the desired product as a white solid (3, 1.48 g, 2.61 mmol, 43% yield). LCMS (ESI): m/z=567.2 [M+H]⁺.

### Preparation of (3S,6S,9S,10aR)-6-amino-9-((3,3-difluoropropyl)(methyl)amino)-3-((S)-6-phenyl-4-azaspiro[2.4]heptane-4-carbonyl)octahydropyrrolo[1,2-a]azocin-5(1H)-one (5)

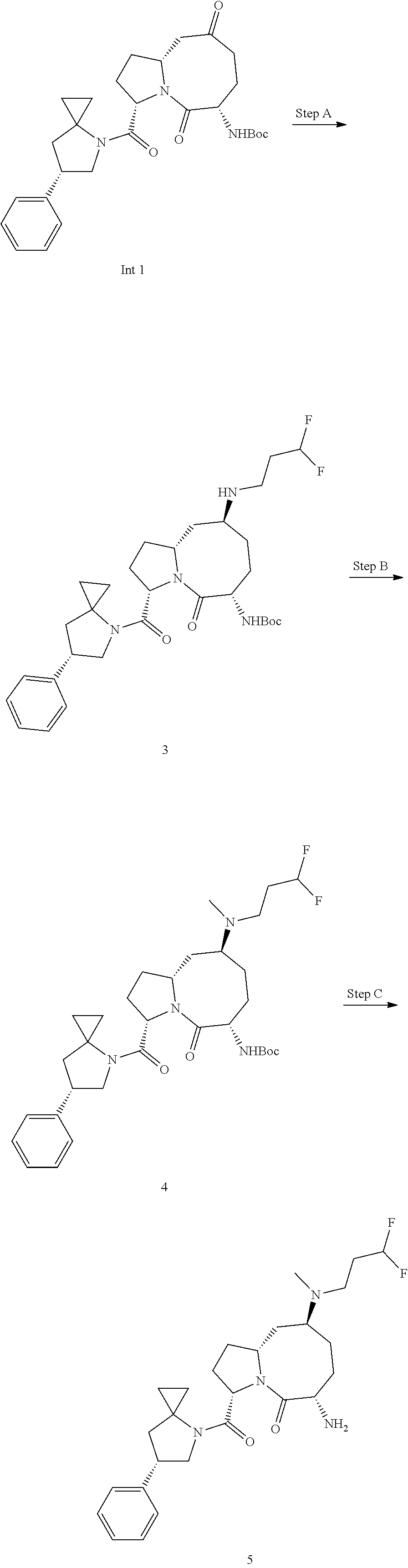 [SMILES: CC(C)(C)OC(=O)N[C@H]1CCC(=O)C[C@H]2CC[C@@H](C(=O)N3C[C@H](c4ccccc4)CC34CC4)N2C1=O ; CC(C)(C)OC(=O)N[C@H]1CC[C@H](NCCC(F)F)C[C@H]2CC[C@@H](C(=O)N3C[C@H](c4ccccc4)CC34CC4)N2C1=O ; CN(CCC(F)F)[C@H]1CC[C@H](NC(=O)OC(C)(C)C)C(=O)N2[C@H](CC[C@H]2C(=O)N2C[C@H](c3ccccc3)CC23CC3)C1 ; CN(CCC(F)F)[C@H]1CC[C@H](N)C(=O)N2[C@H](CC[C@H]2C(=O)N2C[C@H](c3ccccc3)CC23CC3)C1]

### Step A: tert-butyl ((3S,6S,10aR)-9-((3,3-difluoropropyl)amino)-5-oxo-3-((S)-6-phenyl-4-azaspiro[2.4]heptane-4-carbonyl)decahydropyrrolo[1,2-a]azocin-6-yl)carbamate

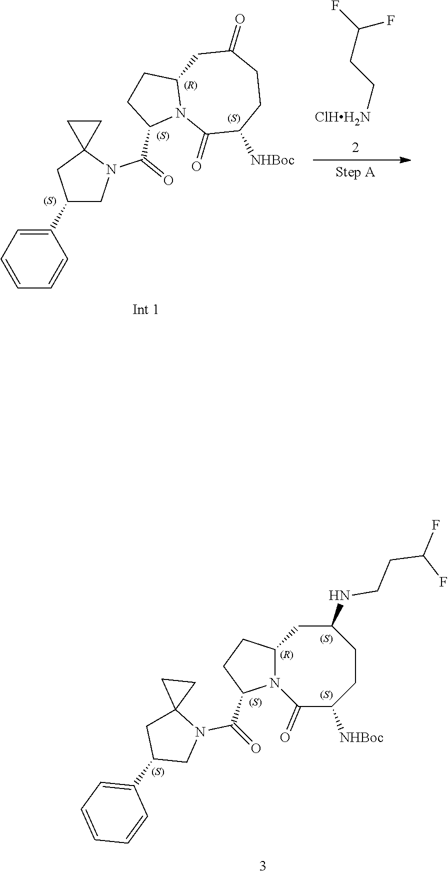

To a mixture of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (Int 1, 1 g, 2.02 mmol, 1.0 eq.) and 3,3-difluoropropan-1-amine hydrochloride (2, 0.35 g, 3.64 mmol, 1.8 eq.) in MeOH (20 mL) was added ZnCl₂ (1.35 g, 10.1 mmol, 5.0 eq.). The resulting mixture was stirred at 55° C. for 1 hr under N₂, then NaBH₃CN (1.27 g, 20.2 mmol, 10.0 eq.) was added to the reaction mixture in five portions and the resulting mixture was stirred at 55° C. overnight under N₂. The reaction mixture was used in the next Step directly without further purification. LCMS (ESI): m/z=575.4[M+H]⁺.

### Step B: tert-butyl ((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamate

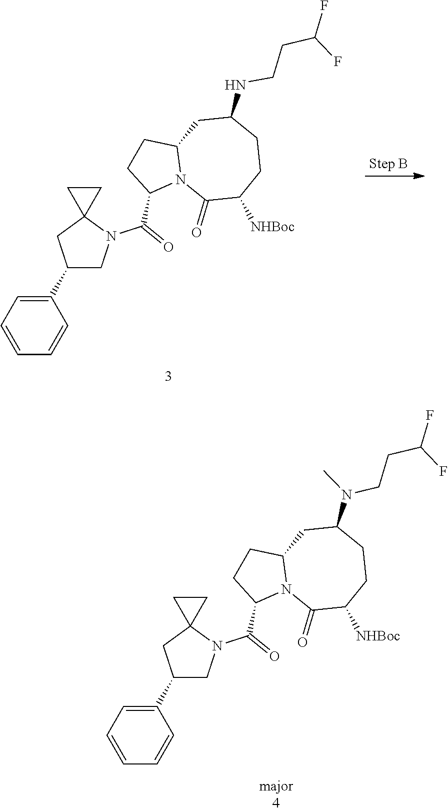 [SMILES: CC(C)(C)OC(=O)N[C@H]1CC[C@H](NCCC(F)F)C[C@H]2CC[C@@H](C(=O)N3C[C@H](c4ccccc4)CC34CC4)N2C1=O ; CN(CCC(F)F)[C@H]1CC[C@H](NC(=O)OC(C)(C)C)C(=O)N2[C@H](CC[C@H]2C(=O)N2C[C@H](c3ccccc3)CC23CC3)C1]

To a mixture of tert-butyl ((3S,6S,10aR)-9-((3,3-difluoropropyl)amino)-5-oxo-3-((S)-6-phenyl-4-azaspiro[2.4]heptane-4-carbonyl)decahydropyrrolo[1,2-a]azocin-6-yl)carbamate (3, Step B above, crude.) were added CH₂O (2 mL, 30 wt % in water) and NaBH₃CN (0.26 g, 4.04 mmol, 2.0 eq.). The resulting mixture was stirred at 55° C. for 30 min under N₂. After completion, the reaction mixture was concentrated under reduced pressure to remove MeOH. The residue was poured into H₂O (50 mL) and then the suspension was filtered. The wet cake was dissolved in DCM (100 mL), dried over anhydrous Na₂SO₄ and concentrated under reduced pressure. The filtrate above was then extracted with DCM (20 mL×3). The organic layers were combined and washed with brine (50 mL), dried over anhydrous Na₂SO₄, and concentrated under reduced pressure. The residue was purified by reverse phase chromatography using a 120 g C18 cartridge eluting with a gradient of 5-80% ACN in water (with 0.1% NH₃H₂O) to afford the desired product as a white solid (4, 700 mg, 12.0 mmol, 59% yield over 2 Steps) LCMS (ESI): m/z=589.2 [M+H]⁺.

### Step C: (3S,6S,9S,10aR)-6-amino-9-((3,3-difluoropropyl)(methyl)amino)-3-((S)-6-phenyl-4-azaspiro[2.4]heptane-4-carbonyl)octahydropyrrolo[1,2-a]azocin-5(1H)-one

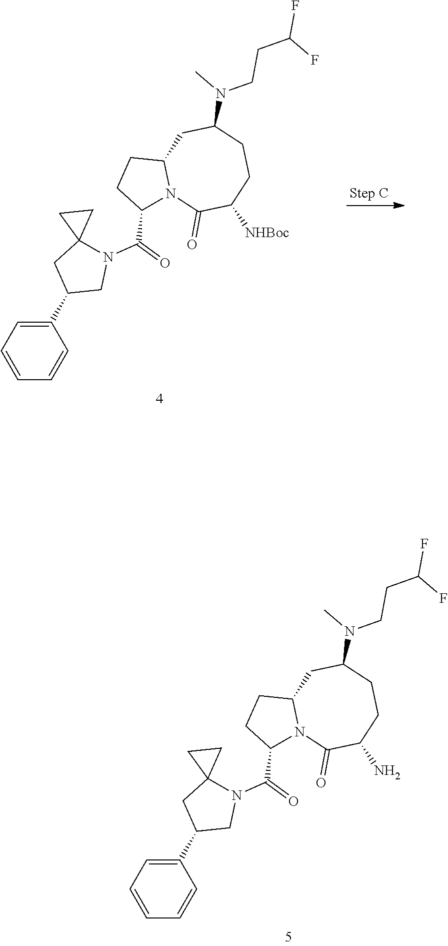 [SMILES: CN(CCC(F)F)[C@H]1CC[C@H](NC(=O)OC(C)(C)C)C(=O)N2[C@H](CC[C@H]2C(=O)N2C[C@H](c3ccccc3)CC23CC3)C1 ; CN(CCC(F)F)[C@H]1CC[C@H](N)C(=O)N2[C@H](CC[C@H]2C(=O)N2C[C@H](c3ccccc3)CC23CC3)C1]

To a solution of tert-butyl ((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamate (4, 600 mg, 1.02 mmol, 1.0 eq.) in DCM (10 mL) was added TFA (3 mL), and the resulting mixture was stirred at room temperature for 2 hrs. After completion, the reaction mixture concentrated under reduced pressure to give crude (3S,6S,9S,10aR)-6-amino-9-((3,3-difluoropropyl)(methyl)amino)-3-((S)-6-phenyl-4-azaspiro[2.4]heptane-4-carbonyl)octahydropyrrolo[1,2-a]azocin-5(1H)-one (TFA salt) (5, 600 mg, quant.) as a white solid, which was used in next Step directly without further purification. LCMS (ESI): m/z=489.4 [M+H]⁺.

### Preparation of propyl (((R)-(2-(((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (prodrug C1), ((R)-(2-(((3S,6S,9S,10aR)-9-((3,3-difluoropropyl)(methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (parent drug A20)

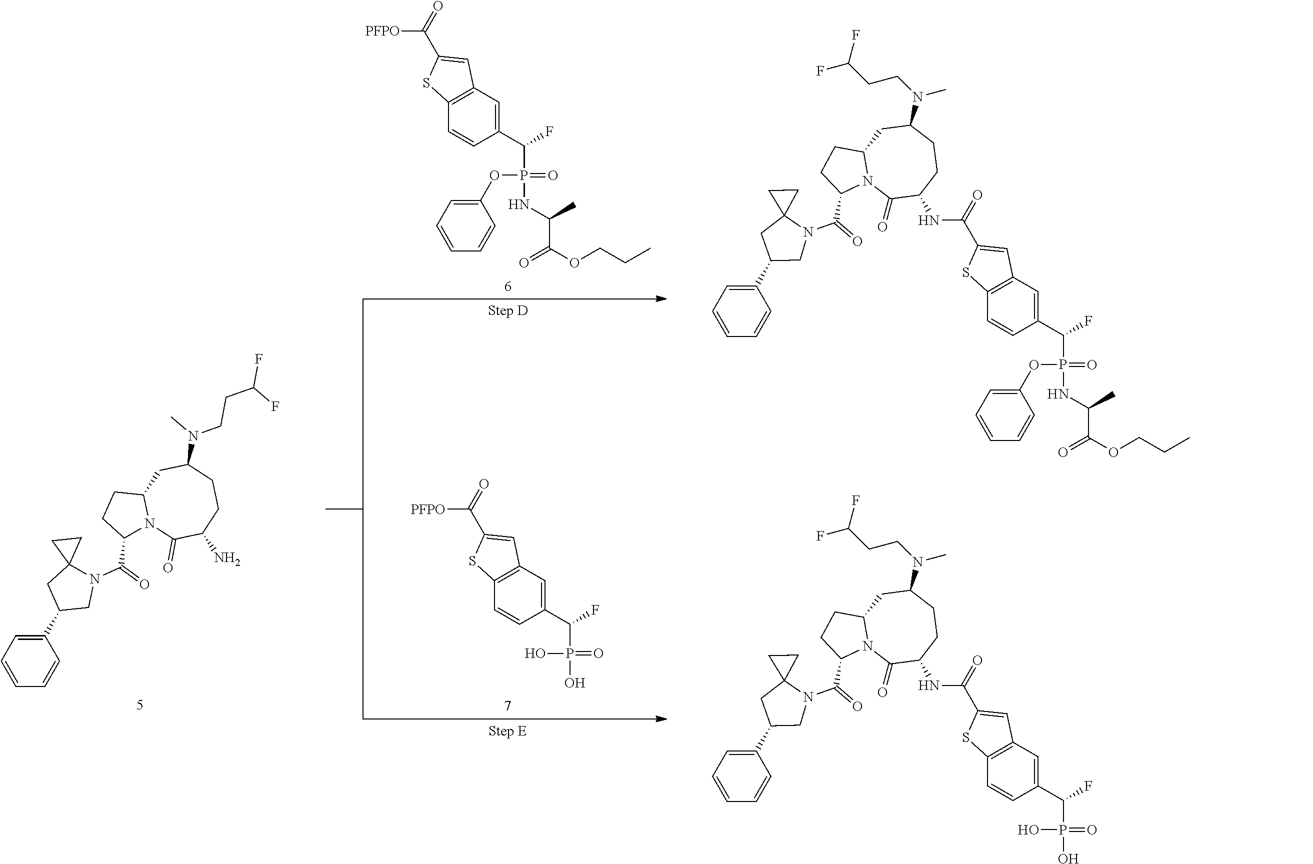

### Step D: propyl (((R)-(2-(((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C1)

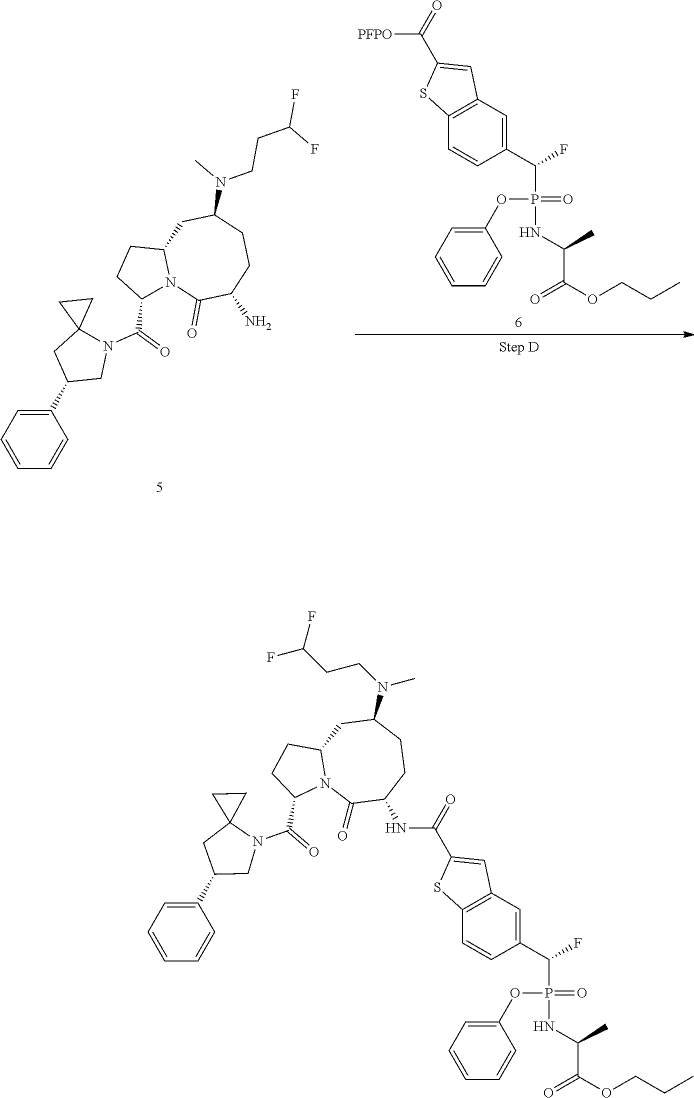

To a solution of (3S,6S,9S,10aR)-6-amino-9-((3,3-difluoropropyl)(methyl)amino)-3-((S)-6-phenyl-4-azaspiro[2.4]heptane-4-carbonyl)octahydropyrrolo[1,2-a]azocin-5(1H)-one (TFA salt) (5, 600 mg, 1.02 mmol, 1.0 eq.) and TEA (515 mg, 5.1 mmol, 5.0 eq.) in DMF (5 mL) was added perfluorophenyl 5-((1R)-fluoro((((S)-1-oxo-1-propoxypropan-2-yl)amino)(phenoxy)phosphoryl)methyl)benzo[b]thiophene-2-carboxylate (6, 650 mg, 1.02 mmol, 1.0 eq.) at room temperature. The resulting mixture was stirred at room temperature for additional 2 hrs. After completion, the reaction mixture was poured into H₂O (50 mL) and then the suspension was filtered. The wet cake was dissolved in DCM (100 mL), dried over anhydrous Na₂SO₄ and concentrated under reduced pressure. The mixture was dissolved in DMF (30 mL) and purified by reverse phase chromatography using a 120 g C18 cartridge eluting with a gradient of 5-80% ACN in water (with 0.1% TFA) to afford the desired product (700 mg, TFA salt) as a white solid. The TFA salt was dissolved in DCM (50 mL) and washed with 5% NaHCO₃ (aq.) (50 mL×4); following by deionized water (50 mL×4). The combined aqueous layers were was extracted with DCM (50 mL×2), then the combined DCM layers concentrated under reduced pressure to give propyl (((R)-(2-(((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (520 mg, 53.7% yield over 2 Steps) as a white solid. ¹H NMR (400 MHz, DMSO) δ 8.82-8.71 (m, 1H), 8.27 (s, 1H), 8.09-8.03 (m, 2H), 7.64-7.57 (m, 1H), 7.38-7.28 (m, 6H), 7.24-7.14 (m, 4H), 6.26-6.20 (m, 1H), 6.16-5.92 (m, 2H), 4.83-4.72 (m, 1H), 4.53-4.42 (m, 1H), 4.36-4.26 (m, 1H), 4.16-4.06 (m, 1H), 3.95-3.70 (m, 4H), 3.58-3.49 (m, 1H), 3.08-2.90 (m, 1H), 2.45-2.32 (m, 2H), 2.25-2.18 (m, 1H), 2.17-1.36 (m, 20H), 1.17-1.09 (m, 3H), 0.85-0.75 (m, 3H), 0.52-0.40 (m, 2H). LCMS (ESI): m/z=950.8 [M+H]⁺.

### Step E: ((R)-(2-(((3S,6S,9S,10a10aR)-9-((3,3-difluoropropyl)(methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A20)

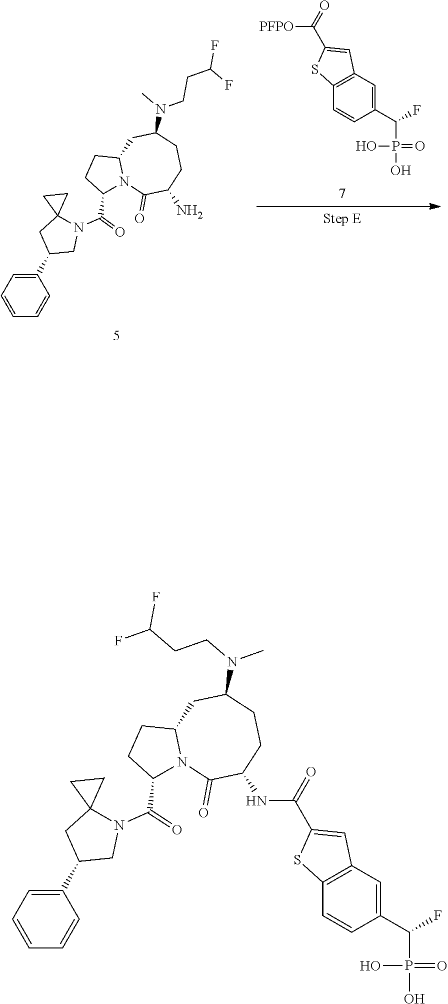

To a solution of (3S,6S,9S,10aR)-6-amino-9-((3,3-difluoropropyl)(methyl)amino)-3-((S)-6-phenyl-4-azaspiro[2.4]heptane-4-carbonyl)octahydropyrrolo[1,2-a]azocin-5(1H)-one (TFA salt) (5, 60 mg, 0.1 mmol, 1.0 eq.) and TEA (51 mg, 0.5 mmol, 5.0 eq.) in DMF (1 mL) was added (R)-(fluoro(2-((perfluorophenoxy)carbonyl)benzo[b]thiophen-5-yl)methyl)phosphonic acid (7, 65 mg, 0.1 mmol, 1.0 eq.) at room temperature. The resulting mixture was stirred at room temperature for additional 2 hrs. After completion, the reaction mixture was purified by reverse phase chromatography using a 40 g C18 cartridge eluting with a gradient of 5-50% ACN in water (with 0.1% TFA) to afford the desired product (75 mg, TFA salt) as a white solid. Then the TFA salt was dissolved in DMF (2 mL) and purified by reverse phase chromatography using a 40 g C18 cartridge eluting with a gradient of 5-50% ACN in water (with 0.1% NH₄HCO₃) to give ((R)-(2-(((3S,6S,9S,10aR)-9-((3,3-difluoropropyl)(methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (free base) (45 mg, 58.5% yield over 2 Steps) as a white solid.

¹H NMR (400 MHz, DMSO) δ 8.87-8.67 (m, 1H), 8.24 (s, 1H), 8.03 (d, J=8.4 Hz, 1H), 7.98 (s, 1H), 7.53 (d, J=8.4 Hz, 1H), 7.38-7.29 (m, 4H), 7.26-7.19 (m, 1H), 6.40-6.03 (m, 1H), 5.91-5.75 (m, 1H), 4.81 (s, 1H), 4.60-4.50 (m, 2H), 4.17-4.09 (m, 1H), 3.85-3.70 (m, 2H), 3.57-3.51 (m, 1H), 3.35-3.19 (m, 2H), 2.80-2.69 (m, 3H), 2.49-2.42 (m, 1H), 2.39-1.70 (m, 14H), 1.56 (s, 1H), 0.59-0.42 (m, 2H). LCMS (ESI): m/z=761.5 [M+H]⁺.

### Preparation of (((R)-(2-(((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(hydroxy)phosphoryl)-L-alanine

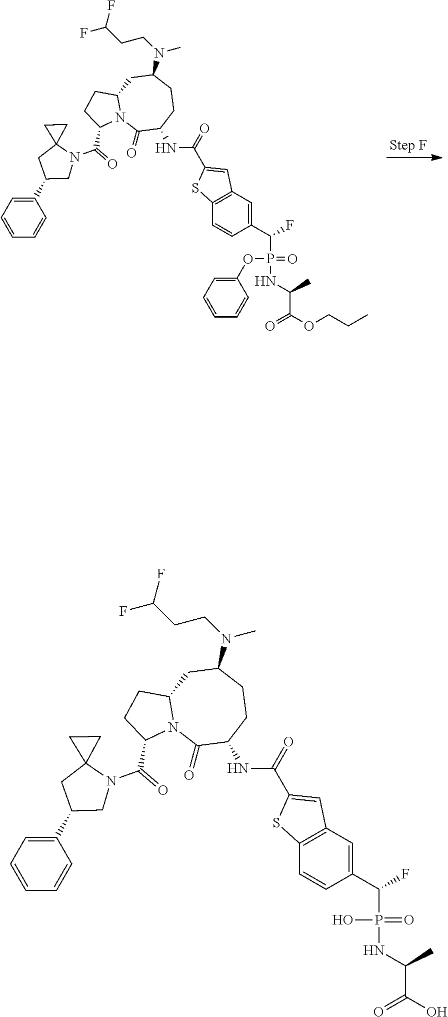 [SMILES: CCCOC(=O)[C@H](C)NP(=O)(Oc1ccccc1)[C@@H](F)c1ccc2sc(C(=O)N[C@H]3CC[C@H](N(C)CCC(F)F)C[C@H]4CC[C@@H](C(=O)N5C[C@H](c6ccccc6)CC56CC6)N4C3=O)cc2c1 ; C[C@H](NP(=O)(O)[C@@H](F)c1ccc2sc(C(=O)N[C@H]3CC[C@H](N(C)CCC(F)F)C[C@H]4CC[C@@H](C(=O)N5C[C@H](c6ccccc6)CC56CC6)N4C3=O)cc2c1)C(=O)O]

### Step F: (((R)-(2-(((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(hydroxy)phosphoryl)-L-alanine (Compound B6)

To a solution of propyl (((R)-(2-(((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (200 mg, 0.21 mmol, 1.00 eq) in THF (3 mL) was added LiOH·H₂O (2 M, 1 mL, 10 eq) at 20° C. and stirred at 20° C. for 2 hrs. LCMS showed that SM was consumed and desired MS (0%-30% method) was detected. The mixture was concentrated to remove methanol. The water phase was purified by prep-HPLC (column: Waters Xbridge C18 150*50 mm*10 um; mobile phase: [water(NH₄HCO₃)-ACN]; gradient:10%-30% B over 10 min) and lyophilized to give (((R)-(2-(((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(hydroxy)phosphoryl)-L-alanine (110 mg, 132 mol, 63% yield,) as a white solid. ¹H NMR (400 MHz, DMSO) δ 8.70 (d, J=7.4 Hz, 1H), 8.20 (s, 1H), 7.92 (d, J=8.1 Hz, 2H), 7.54 (d, J=8.5 Hz, 1H), 7.38-7.28 (m, 4H), 7.22 (t, J=7.1 Hz, 1H), 6.10 (t, J=57.2 Hz, 1H), 5.61 (dd, J=45.7, 7.6 Hz, 1H), 4.81-4.69 (m, 1H), 4.49 (t, J=8.3 Hz, 1H), 4.34 (s, 1H), 4.10 (t, J=8.6 Hz, 1H), 3.74 (t, J=9.8 Hz, 1H), 3.58-3.37 (m, 2H), 3.28-2.98 (m, 2H), 2.45-2.30 (m, 2H), 2.29-2.15 (m, 3H), 2.14-2.05 (m, 2H), 2.04-1.93 (m, 4H), 1.92-1.35 (m, 9H), 1.06 (d, J=6.8 Hz, 3H), 0.53-0.41 (m, 2H). LCMS: m/z=832.5 (M+H)+.

### Representative Procedures for Syntheses of Cores:

### Synthesis of (3S,6S,10aS)-6-((tert-butoxycarbonyl)amino)-5-oxodecahydropyrrolo[1,2-a]azocine-3-carboxylic acid

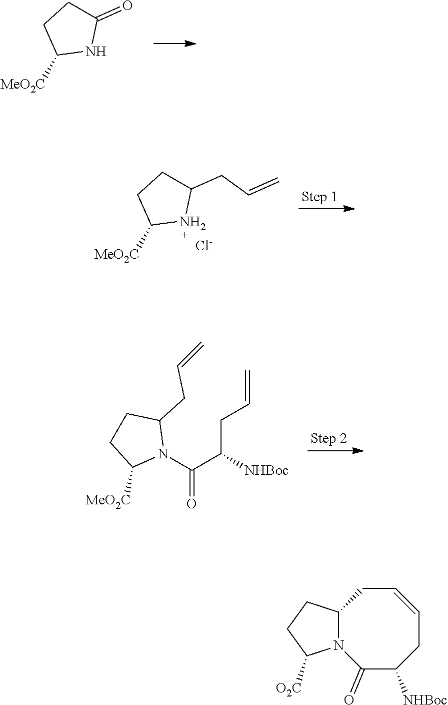

### Step 1: Preparation of methyl (2S)-5-allyl-1-((S)-2-((tert-butoxycarbonyl)amino)pent-4-enoyl)pyrrolidine-2-carboxylate

To a cooled (0° C.) solution of methyl (2S)-5-allylpyrrolidine-2-carboxylate hydrochloride (200 g, 972 mmol, 1.00 eq) and (S)-2-((tert-butoxycarbonyl)amino)pent-4-enoic acid (209 g, 972 mmol, 1.00 eq) in CH₂Cl₂ (1.60 L) was added Et₃N (406 mL, 2.92 mol, 3.00 eq) and 2-chloro-1-methylpyridinium iodide (CMPI) (273 g, 1.07 mol, 1.10 eq). The solution was warmed to 25° C. and stirred for 1 h. The mixture was poured into water (5.0 L), extracted with CH₂Cl₂ (2.00 L×3). The combined organic layers were washed with brine (2.0 L), dried over Na₂SO₄, filtered and concentrated under reduced pressure. Six individual batches of equal scale were performed in parallel and combined during work up. The resulting residue was purified by column chromatography (petroleum ether:EtOAc=100:1 to 10:1) to give methyl (2S)-5-allyl-1-((S)-2-((tert-butoxycarbonyl)amino)pent-4-enoyl)pyrrolidine-2-carboxylate (1.18 kg, 3.22 mol, 55.2% yield) as yellow oil. ¹H NMR (400 MHz, CDCl₃) δ 5.84-5.78 (m, 2H), 5.17-4.99 (m, 5H), 4.50-4.34 (m, 3H), 3.76-3.70 (m, 3H), 2.50-2.15 (m, 6H), 1.96-1.91 (m, 2H), 1.41 (s, 9H).

### Step 2: Preparation of methyl (3S,6S,10aR,Z)-6-((tert-butoxycarbonyl)amino)-5-oxo-1,2,3,5,6,7,10,10a-octahydropyrrolo[1,2-a]azocine-3-carboxylate

To a solution of methyl (2S)-5-allyl-1-((S)-2-((tert-butoxycarbonyl)amino)pent-4-enoyl)pyrrolidine-2-carboxylate (200 g, 546 mmol, 1.00 eq) in CH₂Cl₂ (2.00 L) was added 1^{st} generation Grubbs catalyst (44.9 g, 54.6 mmol, 0.10 eq) at 25° C. The solution was subsequently heated to 50° C. and stirred for 36 h. Six individual batches of equal scale were performed in parallel and combined during work up. The combined reaction mixtures were concentrated to give a residue. The residue was purified by column chromatography (petroleum ether:EtOAc=100:1 to 0:1) twice to give a crude product. The crude product was triturated with petroleum ether (2.00 L) for 12 h and filtered. The filter cake was dried under reduced pressure to give methyl (3S,6S,10aR,Z)-6-((tert-butoxycarbonyl)amino)-5-oxo-1,2,3,5,6,7,10,10a-octahydropyrrolo[1,2-a]azocine-3-carboxylate (510 g, 1.40 mol, 51.3% yield, 92.9% purity) as a solid. LCMS (ESI) m/z=361.2 [M+Na]⁺; ¹H NMR (400 MHz, CDCl₃) δ 5.82-5.80 (m, 1H), 5.73-5.71 (m, 1H), 5.58-5.56 (m, 1H), 4.88-4.85 (m, 1H), 4.52-4.49 (m, 1H), 4.16-4.14 (m, 1H), 3.71 (s, 3H), 2.81-2.74 (m, 2H), 2.45-2.38 (m, 1H), 2.33-2.24 (m, 1H), 2.14-2.05 (m, 2H), 1.98-1.93 (m, 2H), 1.43 (s, 9H).

### Synthesis of (3S,6S,9R,10aR)-6-{[(tert-butoxy)carbonyl]amino}-9-hydroxy-5-oxo-decahydropyrrolo[1,2-a]azocine-3-carboxylic acid, (3S,6S,9S,10aR)-6-((tert-butoxycarbonyl)amino)-9-hydroxy-5-oxodecahydropyrrolo[1,2-a]azocine-3-carboxylic acid, (3S,6S,8S,10aR)-6-((tert-butoxycarbonyl)amino)-8-hydroxy-5-oxodecahydropyrrolo[1,2-a]azocine-3-carboxylic acid, (3S,6S,8R,10aR)-6-((tert-butoxycarbonyl)amino)-8-hydroxy-5-oxodecahydropyrrolo[1,2-a]azocine-3-carboxylic acid

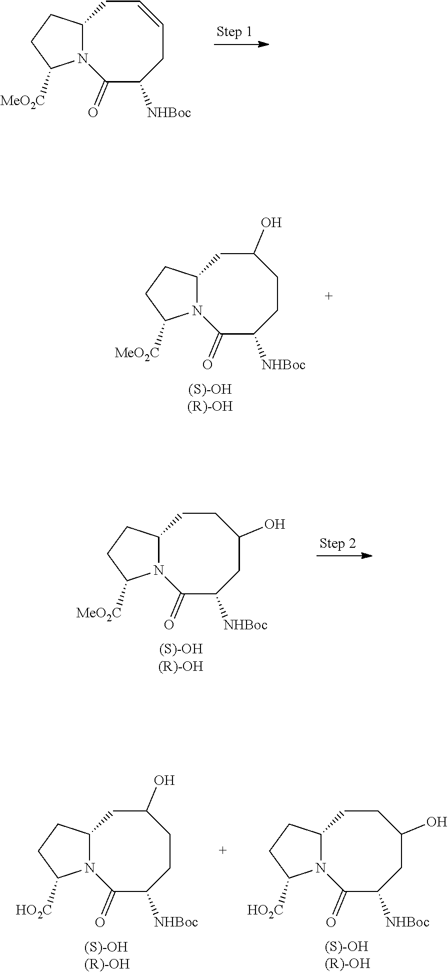

### Step 1: Preparation of methyl (3S,6S,10aR)-6-((tert-butoxycarbonyl)amino)-9-hydroxy-5-oxodecahydropyrrolo[1,2-a]azocine-3-carboxylate and methyl (3S,6S,10aR)-6-((tert-butoxycarbonyl)amino)-8-hydroxy-5-oxodecahydropyrrolo[1,2-a]azocine-3-carboxylate

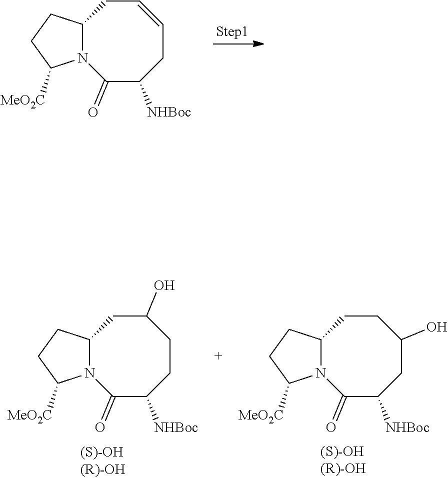

Methyl (3S,6S,10aR)-6-((tert-butoxycarbonyl)amino)-9-hydroxy-5-oxodecahydropyrrolo[1,2-a]azocine-3-carboxylate and methyl (3S,6S,10aR)-6-((tert-butoxycarbonyl)amino)-8-hydroxy-5-oxodecahydropyrrolo[1,2-a]azocine-3-carboxylate were prepared starting from methyl (3S,6S,10aR,Z)-6-((tert-butoxycarbonyl)amino)-5-oxo-1,2,3,5,6,7,10,10a-octahydropyrrolo[1,2-a]azocine-3-carboxylate and utilizing similar protocols established in Journal of Medicinal Chemistry (2010), 53(17), 6361-6367.

The mixture of alcohol diastereoisomers and regioisomers were separated by reverse phase chromatography [C18 cartridge eluting with a gradient of 5-40% MeOH in water (with 0.1% formic acid)] followed by chiral SFC separation [Lux i-Cellulose-5 21.2×250 mm 5 um column, column temp=40° C., flow rate 75 mL/min, 20% MeOH, cycle time: 5 min]. A representative reaction starting from methyl (3S,6S,10aR,Z)-6-((tert-butoxycarbonyl)amino)-5-oxo-1,2,3,5,6,7,10,10a-octahydropyrrolo[1,2-a]azocine-3-carboxylate (4.3 g, 12.7 mmol) afforded the following products:

Peak 1: Methyl (3S,6S,9R,10aR)-6-{[(tert-butoxy)carbonyl]amino}-9-hydroxy-5-oxo-decahydropyrrolo[1,2-a]azocine-3-carboxylate (760 mg, 2.13 mmol, 16.8% yield) as a white solid. LCMS (ESI) m/z=357.2 [M+H]⁺.

Peak 2: Methyl (3S,6S,9S,10aR)-6-{[(tert-butoxy)carbonyl]amino}-9-hydroxy-5-oxo-decahydropyrrolo[1,2-a]azocine-3-carboxylate (375 mg, 19% yield) as a clear thick oil. LCMS (ESI) m/z=357.2 [M+H]⁺.

Peak 3: Methyl (3S,6S,8R,10aR)-6-{[(tert-butoxy)carbonyl]amino}-8-hydroxy-5-oxo-decahydropyrrolo[1,2-a]azocine-3-carboxylate (795 mg, 40% yield) as a white foam. LCMS (ESI) m/z=357.2 [M+H]⁺.

Peak 4: Methyl (3S,6S,8S,10aR)-6-{[(tert-butoxy)carbonyl]amino}-8-hydroxy-5-oxo-decahydropyrrolo[1,2-a]azocine-3-carboxylate (600 mg, 30% yield) as a white solid. LCMS (ESI) m/z=357.2 [M+H]⁺.

### Step 2: Preparation of (3S,6S,9R,10aR)-6-{[(tert-Butoxy)carbonyl]amino}-9-hydroxy-5-oxo-decahydropyrrolo[1,2-a]azocine-3-carboxylic acid

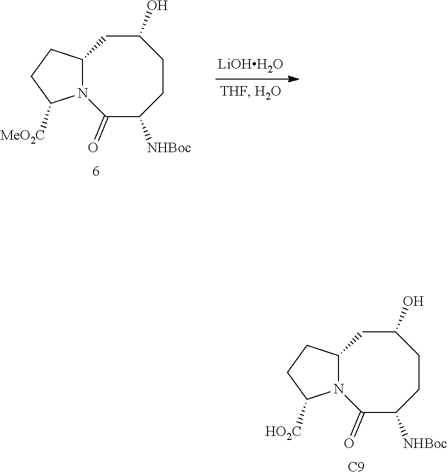

To a solution of methyl (3S,6S,9R,10aR)-6-{[(tert-butoxy)carbonyl]amino}-9-hydroxy-5-oxo-decahydropyrrolo[1,2-a]azocine-3-carboxylate (Peak 1) (1.08 g, 3.03 mmol, 1 eq) in a mixture of THF (24 mL) and water (8 mL) was added LiOH—H₂O (380 mg, 9.08 mmol, 3.0 eq). The reaction mixture was stirred at room temperature for 2 hours. The reaction was subsequently concentrated under reduced pressure to remove tetrahydrofuran. The crude residue was purified by reverse phase chromatography [C18 cartridge eluting with a gradient of 5-60% Acetonitrile in water (with 0.1% formic acid)] to give (3S,6S,9R,10aR)-6-{[(tert-butoxy)carbonyl]amino}-9-hydroxy-5-oxo-decahydropyrrolo[1,2-a]azocine-3-carboxylic acid (780 mg, 76% yield) as a white solid. LCMS (ESI) m/z=343.2 [M+H]⁺.

The intermediates in the table below were prepared according to the general procedure described above starting from the appropriate starting materials.

| Name | Structure | LCMS |
|---|---|---|
| (3S,6S,9S,10aR)-6-((tert- butoxycarbonyl)amino)-9-hydroxy- 5-oxodecahydropyrrolo[1,2- a]azocine-3-carboxylic acid | 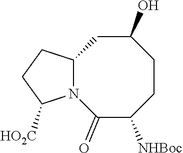 [SMILES: CC(C)(C)OC(=O)N[C@H]1CC[C@H](O)C[C@H]2CC[C@@H](C(=O)O)N2C1=O] Peak 2 of starting material core used for synthesis | 343.2 [M + H]⁺ |
| (3S,6S,8S,10aR)-6-((tert- butoxycarbonyl)amino)-8-hydroxy- 5-oxodecahydropyrrolo[1,2- a]azocine-3-carboxylic acid | 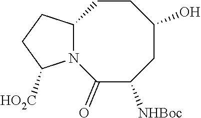 [SMILES: CC(C)(C)OC(=O)N[C@H]1C[C@@H](O)CC[C@H]2CC[C@@H](C(=O)O)N2C1=O] Peak 4 of starting material core used for synthesis | 343.2 [M + H]⁺ |
| (3S,6S,8R,10aR)-6-((tert- butoxycarbonyl)amino)-8-hydroxy- 5-oxodecahydropyrrolo[1,2- a]azocine-3-carboxylic acid | 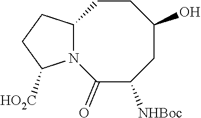 [SMILES: CC(C)(C)OC(=O)N[C@H]1C[C@H](O)CC[C@H]2CC[C@@H](C(=O)O)N2C1=O] Peak 3 of core building block used for synthesis | 343.2 [M + H]⁺ |

### Synthesis of methyl (3S,6S,10aR)-6-{[(tert-butoxy)carbonyl]amino}-5,9-dioxo-decahydropyrrolo[1,2-a]azocine-3-carboxylate and methyl (3S,6S,10aR)-6-{[(tert-butoxy)carbonyl]amino}-5,8-dioxo-decahydropyrrolo[1,2-a]azocine-3-carboxylate

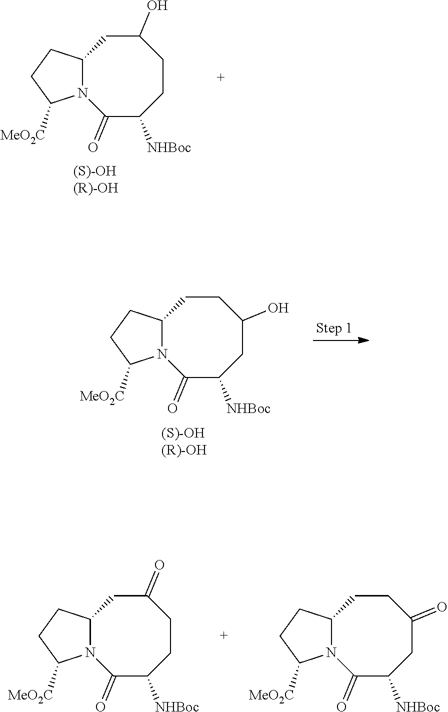

Methyl (3S,6S,10aR)-6-{[(tert-butoxy)carbonyl]amino}-5,9-dioxo-decahydropyrrolo[1,2-a]azocine-3-carboxylate and methyl (3S,6S,10aR)-6-{[(tert-butoxy)carbonyl]amino}-5,8-dioxo-decahydropyrrolo[1,2-a]azocine-3-carboxylate were prepared using the protocol described in US 2009/0123480. The crude reaction mixture was purified by reverse phase chromatography using a [C18 cartridge eluting with a gradient of 5-100% acetonitrile in water (with 0.1% FA)] to give a mixture of ketone isomers (1.2 g) as a beige solid. The resultant ketone isomers were separated chiral SFC separation (Column Lux i-Cellulose-5 21.2×250 mm 5 um column, flow rate 75 mL/min, 15% MeOH) to give methyl (3S,6S,10aR)-6-{[(tert-butoxy)carbonyl]amino}-5,9-dioxo-decahydropyrrolo[1,2-a]azocine-3-carboxylate (9) (424 mg, 35.6% yield) as a white solid and methyl (3S,6S,10aR)-6-{[(tert-butoxy)carbonyl]amino}-5,8-dioxo-decahydropyrrolo[1,2-a]azocine-3-carboxylate (433 mg, 36.3% yield) as a white solid.

Methyl (3S,6S,10aR)-6-{[(tert-butoxy)carbonyl]amino}-5,9-dioxo-decahydropyrrolo[1,2-a]azocine-3-carboxylate: LCMS (ESI) m/z=299.1 [M+H]⁺; ¹H NMR: (400 MHz, CDCl₃) δ 5.27 (d, J=8.1 Hz, 1H), 4.59 (t, J=8.7 Hz, 1H), 4.45-4.31 (m, 2H), 3.80 (s, 3H), 3.20 (td, J=12.5, 4.9 Hz, 1H), 3.06 (t, J=12.0 Hz, 1H), 2.47-2.15 (m, 5H), 2.13-2.00 (m, 1H), 1.87 (dd, J=12.1, 7.0 Hz, 1H), 1.68-1.62 (m, 1H), 1.41 (s, 9H).

Methyl (3S,6S,10aR)-6-{[(tert-butoxy)carbonyl]amino}-5,8-dioxo-decahydropyrrolo[1,2-a]azocine-3-carboxylate: LCMS (ESI) m/z=299.1 [M+H]⁺; ¹H NMR: (400 MHz, CDCl₃) δ 5.56 (d, J=7.6 Hz, 1H), 5.13 (ddd, J=12.2, 7.6, 4.9 Hz, 1H), 4.51 (t, J=8.8 Hz, 1H), 4.33-4.23 (m, 1H), 3.73 (s, 3H), 3.07 (dd, J=13.8, 4.5 Hz, 1H), 2.96 (td, J=11.9, 2.9 Hz, 1H), 2.65-2.50 (m, 2H), 2.36-1.91 (m, 4H), 1.90-1.76 (m, 2H), 1.45 (s, 9H).

### Representative Procedures for Syntheses of Pyrrolidine Groups:

### Synthesis of (rel-trans)-6-phenyl-4-azaspiro[2.4]heptane-7-carbonitrile TFA salt

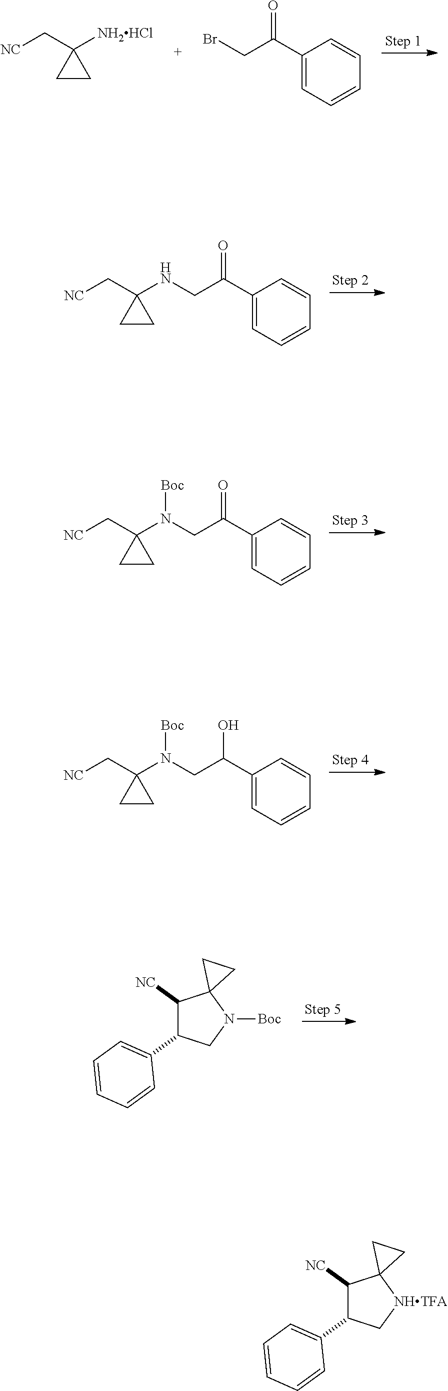

### Step 1: 2-{1-[(2-oxo-2-phenylethyl)amino]cyclopropyl}acetonitrile

To a solution of 2-(1-aminocyclopropyl)acetonitrile hydrochloride (10.7 g, 81.0 mmol, 1 eq) in N,N-dimethylformamide (250 mL) were added 2-bromo-1-phenylethan-1-one (16.1 g, 81.0 mmol, 1 eq) followed by potassium phosphate tribasic (42.8 g, 202 mmol, 2.5 eq). The mixture was stirred at room temperature for 4 h. A yellowish mixture was observed over time. The reaction was quenched with the addition of 1 N HCl (600 mL, pH=1) at room temperature. The aqueous solution was washed with EtOAc (2×500 mL) then basified with 1 N NaOH aqueous solution (until pH=9, 600 mL) then extracted with EtOAc (2×600 mL). The combined organic extracts were washed with brine (2×300 mL), dried over sodium sulfate, filtered and concentrated under reduced pressure to give crude 2-{1-[(2-oxo-2-phenylethyl)amino]cyclopropyl}acetonitrile (12.5 g, 72%) as a clear pale orange liquid. LCMS (ESI) m/z=215.2 [M+H]⁺. 1H NMR (400 MHz, DMSO-d6) δ 7.97 (d, J=7.6 Hz, 2H), 7.66 (t, J=7.1 Hz, 1H), 7.54 (t, J=7.1 Hz, 2H), 4.25 (s, 2H), 2.79 (s, 2H), 0.76-0.70 (m, 2H), 0.61-0.56 (m, 1H).

### Step 2: tert-butyl N-[1-(cyanomethyl)cyclopropyl]-N-(2-oxo-2-phenylethyl)carbamate

To a solution of 2-{1-[(2-oxo-2-phenylethyl)amino]cyclopropyl}acetonitrile (12.5 g, 58.3 mmol, 1 eq) in THF (150 mL) were added water (150 mL) and sodium bicarbonate (4.89 g, 58.3 mmol, 1.0 eq) followed by di-tert-butyl dicarbonate (25.3 g, 116 mmol, 2 eq). The reaction was stirred for 48 h at room temperature. The reaction was concentrated under reduced pressure (to remove THF) then diluted with EtOAc (250 mL) and water (50 mL). The phases were separated and the aqueous phase was back-extracted with EtOAc (2×250 mL). The combined organic layers were dried with sodium sulfate, filtered and concentrated under reduced pressure. The crude residue was purified flash-chromatography on silica gel eluting with 1-25% EtOAc in heptane to give tert-butyl N-[1-(cyanomethyl)cyclopropyl]-N-(2-oxo-2-phenylethyl)carbamate (14.3 g, 78%) as a white solid. LCMS (ESI) m/z=215.2 (M-Boc+2H), 259.2 (M-t-Bu+2H). ¹H NMR (400 MHz, DMSO-d₆ at 80° C.) δ 7.97 (d, J=7.2 Hz, 2H), 7.67 (t, J=7.1 Hz, 1H), 7.55 (t, J=7.1 Hz, 2H), 4.73 (s, 2H), 2.87 (s, 2H), 1.54-1.24 (m, 9H), 1.04-0.99 (m, 2H), 0.94-0.90 (m, 2H).

### Step 3: tert-butyl N-[1-(cyanomethyl)cyclopropyl]-N-(2-hydroxy-2-phenylethyl)carbamate

To a solution of tert-butyl N-[1-(cyanomethyl)cyclopropyl]-N-(2-oxo-2-phenylethyl)carbamate (14.33 g, 45.4 mmol, 1 eq) in methanol (260 mL) at 0° C. was added sodium borohydride (1.71 g, 45.4 mmol, 1.0 eq) portion wise over 10 min. The reaction was stirred for 20 min at room temperature. The reaction was quenched with 1 N HCl (100 mL) and concentrated under reduced pressure (to remove MeOH). The crude residue was extracted with EtOAc (3×200 mL). The combined organic layers were over sodium sulfate, filtered and concentrated under reduced pressure to give tert-butyl N-[1-(cyanomethyl)cyclopropyl]-N-(2-hydroxy-2-phenylethyl)carbamate (14.7 g, 100%) as a clear pale pink oil. 1H NMR (400 MHz, DMSO-d6 at 80° C.) δ 7.38-7.23 (m, 5H), 5.51 (br. s, 1H), 4.89-4.72 (m, 1H), 3.39-3.27 (m, 1H), 3.21-3.13 (m, 1H), 3.04-2.90 (m, 1H), 2.68-2.60 (m, 1H), 1.48 (s, 9H), 0.83-0.65 (m, 4H).

### Step 4: tert-butyl (rel-trans)-7-cyano-6-phenyl-4-azaspiro[2.4]heptane-4-carboxylate

To a solution of tert-butyl N-[1-(cyanomethyl)cyclopropyl]-N-(2-hydroxy-2-phenylethyl)carbamate (12 g, 37.9 mmol, 1 eq) in THF (650 mL) at −15° C. were added diethyl phosphorochloridate (5.74 mL, 39.7 mmol, 1.05 eq) followed by dropwise addition of 1 M LiHMDS in THF (94.7 mL, 94.7 mmol, 2.5 eq) over 30 min. The reaction was stirred at −15° C. for 30 min. The reaction was quenched with 1N HCl (200 mL) and concentrated under reduced pressure (to remove THF). The crude residue was purified flash-chromatography on silica gel eluting with 1-8% EtOAc in heptane to give rac-tert-butyl (rel-trans)-7-cyano-6-phenyl-4-azaspiro[2.4]heptane-4-carboxylate (3.7 g, 33%) as a clear oil. LCMS (ESI) m/z=243.2 (M-t-Bu+2H)⁺. ¹H NMR (400 MHz, DMSO-d₆) δ 7.59-7.21 (m, 5H), 4.01 (d, J=10.5 Hz, 1H), 3.90 (dd, J=10.5, 7.8 Hz, 1H), 3.69 (q, J=10.2 Hz, 1H), 3.45 (t, J=10.0 Hz, 1H), 1.96-1.83 (m, 1H), 1.48-1.41 (m, 1H), 1.40-1.32 (m, 9H), 0.92-0.77 (m, 1H), 0.74-0.61 (m, 1H).

rac-tert-butyl (6R,7S)-7-cyano-6-phenyl-4-azaspiro[2.4]heptane-4-carboxylate was submitted to chiral SFC separation (SFC conditions: Column Lux Amylose 1, 30×250 mm 5 um column, 9.25 mg/inj, concentration 18.5 mg/mL, Column T=40° C., Flow rate 100 mL/min, 10% MeOH, cycle time: 2 min) to give tert-butyl (6R,7S)-7-cyano-6-phenyl-4-azaspiro[2.4]heptane-4-carboxylate (1.57 g, 5.26 mmol, 42% recovery, 99.9% ee) as a white solid and tert-butyl (6S,7R)-7-cyano-6-phenyl-4-azaspiro[2.4]heptane-4-carboxylate (1.57 g, 0.70 mmol, 42% recovery, 99.9% ee) as a white solid.

The assignment of absolute stereochemical configuration was made by comparing experimental vibrational circular dichroism (VCD) spectra with theoretical VCD spectra obtained from DFT calculations.

### Step 5: (rel-trans)-6-phenyl-4-azaspiro[2.4]heptane-7-carbonitrile TFA salt

To a solution of a tert-butyl (rel-trans)-7-cyano-6-phenyl-4-azaspiro[2.4]heptane-4-carboxylate (32 mg, 0.1072 mmol, 1 eq) in methylene chloride (4 mL) was added TFA (1 mL). The reaction was stirred at room temperature for 2 h. The reaction was concentrated under reduced pressure to give crude (rel-trans)-6-phenyl-4-azaspiro[2.4]heptane-7-carbonitrile TFA salt (21.2 mg, 99%) as a thick oil. LCMS (ESI) m/z=229.2 [M+H]⁺.

### Synthesis of racemic-(6S,7R)-6-(3-methoxyphenyl)-4-azaspiro[2.4]heptane-7-carbonitrile

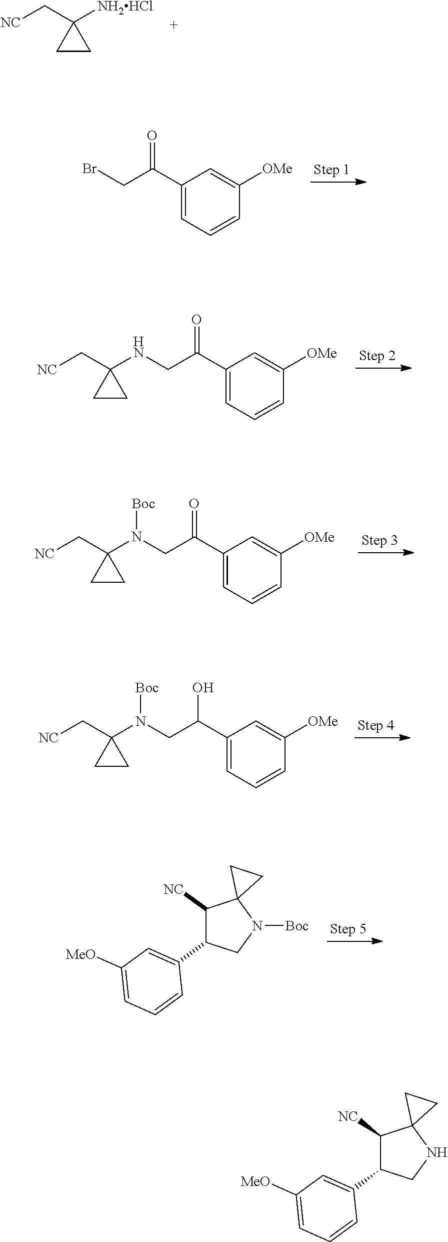

### Step 1: Preparation of 2-(1-((2-(3-methoxyphenyl)-2-oxoethyl)amino)cyclopropyl)acetonitrile

To a solution of 2-(1-aminocyclopropyl)acetonitrile hydrochloride (367 mg, 3.82 mmol, 1.0 eq) in DMF (10 mL) were added 2-bromo-1-(3-methoxyphenyl)ethan-1-one (918 mg, 4.01 mmol, 1.05 eq) and NaHCO₃ (801 mg, 9.54 mmol, 2.5 eq). The mixture was stirred at room temperature for 20 h. The reaction was diluted with MeCN and filtered. The filtrate was concentrated under reduced pressure. The crude solution was purified by reverse phase chromatography on a Cis cartridge eluting with 5-60% MeCN in basic water (10 mM NH₄HCO₃ (pH=10) buffer). The pure fractions were concentrated under reduced pressure to give 2-(1-((2-(3-methoxyphenyl)-2-oxoethyl)amino)cyclopropyl)acetonitrile (522 mg, 55.9%) as an orange oil. LCMS (ESI) m/z=245.1 [M+H]⁺. ¹H NMR (400 MHz, CDCl₃) δ 7.53-7.51 (m, 1H), 7.49-7.46 (m, 1H), 7.38 (t, J=7.9 Hz, 1H), 7.16-7.10 (m, 1H), 4.25 (s, 2H), 3.86 (s, 3H), 2.58 (s, 2H), 0.89-0.84 (m, 2H), 0.74-0.70 (m, 2H).

The intermediates in the table below were prepared using the method described above in Step 1 for the preparation of 2-(1-((2-(3-methoxyphenyl)-2-oxoethyl)amino)cyclopropyl)acetonitrile utilizing the appropriate starting materials and modifications.

| Name | Structure | LCMS |
|---|---|---|
| 2-(1-((2-(3-fluorophenyl)-2- oxoethyl)amino)cyclopropyl)acetonitrile | 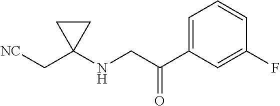 [SMILES: N#CCC1(NCC(=O)c2cccc(F)c2)CC1] | 233.2 [M + H]⁺ |

### Step 2: Preparation of tert-butyl (1-(cyanomethyl)cyclopropyl)(2-(3-methoxyphenyl)-2-oxoethyl)carbamate

To a solution of 2-(1-((2-(3-methoxyphenyl)-2-oxoethyl)amino)cyclopropyl)acetonitrile (522 mg, 2.13 mmol, 1 eq) in THF (1 mL) were added water (1 mL) and NaHCO₃ (178 mg, 2.13 mmol, 1.0 eq) followed by di-tert-butyl dicarbonate (929 mg, 4.26 mmol, 2 eq) at 0° C. The reaction was warmed up to room temperature and stirred for 48 h at room temperature. The reaction was diluted with MeCN and filtered. The filtrate was concentrated under reduced pressure. The crude residue was purified by reverse phase chromatography on a Cis cartridge eluting with 5-80% MeCN in water (with 0.1% formic acid) to give tert-butyl (1-(cyanomethyl)cyclopropyl)(2-(3-methoxyphenyl)-2-oxoethyl)carbamate (400 mg, 54.5%) as a sticky oil. LCMS (ESI) m/z=245.2 [(M-Boc)⁺2H]⁺.

The intermediates in the table below were prepared using the method described above in Step 2 for the preparation of tert-butyl (1-(cyanomethyl)cyclopropyl)(2-(3-methoxyphenyl)-2-oxoethyl)carbamate utilizing the appropriate starting materials and modifications.

| Name | Structure | LCMS |
|---|---|---|
| tert-butyl (1-(cyanomethyl)cyclopropyl)(2- (3-fluorophenyl)-2-oxoethyl)carbamate | 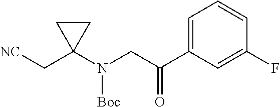 [SMILES: CC(C)(C)OC(=O)N(CC(=O)c1cccc(F)c1)C1(CC#N)CC1] | 277.2 [(M-tBu) + 2H]⁺ |

### Step 3: Preparation of tert-butyl (1-(cyanomethyl)cyclopropyl)(2-hydroxy-2-(3-methoxyphenyl)ethyl)carbamate

To a solution of tert-butyl (1-(cyanomethyl)cyclopropyl)(2-(3-methoxyphenyl)-2-oxoethyl)carbamate (193 mg, 0.5603 mmol, 1 eq) in methanol (10 mL) at 0° C. was added NaBH₄ (21.1 mg, 0.5603 mmol, 1.0 eq). The reaction was stirred for 2 h at room temperature. The reaction was quenched with 1 N HCl (1 mL) and concentrated under reduced pressure. The crude residue was purified by reverse phase chromatography on a Cis cartridge eluting with 5-60% MeCN in water (with 0.1% formic acid) to give tert-butyl (1-(cyanomethyl)cyclopropyl)(2-hydroxy-2-(3-methoxyphenyl)ethyl)carbamate (144 mg, 74.2%) as a thick clear oil. ¹H NMR (400 MHz, DMSO-d₆) δ 7.26 (t, J=7.8 Hz, 1H), 6.91-6.80 (m, 3H), 5.56-5.48 (m, 1H), 4.88-4.71 (m, 1H), 3.75 (s, 3H), 3.40-3.30 (m, 1H), 3.19-3.10 (m, 1H), 3.04-2.92 (m, 1H), 2.73-2.61 (m, 1H), 1.46 (s, 9H), 1.32-1.20 (m, 1H), 0.84-0.62 (m, 3H).

The intermediates in the table below were prepared using the method described above in Step 3 for the preparation of tert-butyl (1-(cyanomethyl)cyclopropyl)(2-hydroxy-2-(3-methoxyphenyl)ethyl)carbamate utilizing the appropriate starting materials and modifications.

| Name | Structure | LCMS | NMR |
|---|---|---|---|
| tert-butyl (1- (cyanomethyl)cyclopropyl)(2- (3-fluorophenyl)-2- hydroxyethyl)carbamate | 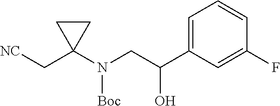 [SMILES: CC(C)(C)OC(=O)N(CC(O)c1cccc(F)c1)C1(CC#N)CC1] | 261.2 [(M-tBu- OH) + 2H]⁺ | |

### Step 4: Preparation of racemic-tert-butyl (6S,7R)-7-cyano-6-(3-methoxyphenyl)-4-azaspiro[2.4]heptane-4-carboxylate

To a solution of tert-butyl (1-(cyanomethyl)cyclopropyl)(2-hydroxy-2-(3-methoxyphenyl)ethyl)carbamate (115 mg, 0.3319 mmol, 1 eq) in THF (10 mL) at −15° C. were added diethyl phosphorochloridate (50.3 μL, 0.3484 mmol, 1.05 eq) followed by dropwise addition of 1 M LiHMDS in THF (829 μL, 0.8297 mmol, 2.5 eq). The reaction was stirred at −15° C. for 2 h. The reaction was quenched with 1 N HCl (1 mL) and concentrated under reduced pressure. The crude residue was purified by reverse phase chromatography on a Cis cartridge eluting with 5-70% MeCN in water (with 0.1% formic acid) to give racemic-tert-butyl (6S,7R)-7-cyano-6-(3-methoxyphenyl)-4-azaspiro[2.4]heptane-4-carboxylate (50.0 mg, 46.2%) as a white solid. LCMS (ESI) m/z=273.2 [(M-t-Bu)⁺2H]⁺. ¹H NMR (400 MHz, C₆D₆) δ 7.03-6.98 (m, 1H), 6.66-6.61 (m, 2H), 6.53-6.49 (m, 1H), 3.72-3.62 (m, 1H), 3.29 (s, 3H), 3.25-3.18 (m, 1H), 3.04-2.96 (m, 1H), 2.71 (d, J=10.5 Hz, 1H), 2.38-1.93 (m, 1H), 1.75-1.47 (m, 1H), 1.37 (s, 9H), 1.09-1.02 (m, 1H), 0.45-0.39 (m, 1H).

The intermediates in the table below were prepared using the method described above in Step 4 for the preparation of racemic-tert-butyl (6S,7R)-7-cyano-6-(3-methoxyphenyl)-4-azaspiro[2.4]heptane-4-carboxylate utilizing the appropriate starting materials and modifications.

| Name | Structure |
|---|---|
| Racemic-tert-butyl (6R,7S)- 7-cyano-6-(3-fluorophenyl)- 4-azaspiro[2.4]heptane-4- carboxylate | 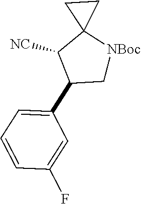 [SMILES: CC(C)(C)OC(=O)N1C[C@@H](c2cccc(F)c2)[C@H](C#N)C12CC2] |

### Step 5: Preparation of racemic-(6S,7R)-6-(3-methoxyphenyl)-4-azaspiro[2.4]heptane-7-carbonitrile

To a solution of a tert-butyl (6R,7S)-7-cyano-6-(3-methoxyphenyl)-4-azaspiro[2.4]heptane-4-carboxylate (44 mg, 0.1339 mmol, 1 eq) in methylene chloride (2 mL) was added TFA (0.5 mL). The reaction was stirred at room temperature for 2 h. The reaction was concentrated under reduced pressure to give crude racemic-(6S,7R)-6-(3-methoxyphenyl)-4-azaspiro[2.4]heptane-7-carbonitrile (30.5 mg, 99%, TFA salt) as a thick oil. LCMS (ESI) m/z=229.2 [M+H]⁺.

The intermediates in the table below were prepared using the method described above in Step 5 for the preparation of racemic-(6S,7R)-6-(3-methoxyphenyl)-4-azaspiro[2.4]heptane-7-carbonitrile utilizing the appropriate starting materials and modifications.

| Name | Structure | LCMS |
|---|---|---|
| Racemic-(6R,7S)-6-(3-fluorophenyl)-4-azaspiro[2.4]heptane-7- carbonitrile (TFA salt) | 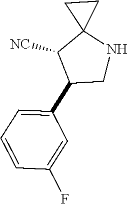 [SMILES: N#C[C@H]1[C@H](c2cccc(F)c2)CNC12CC2] | 217.2 [M + H]⁺ |

### Synthesis of racemic-trans-6-(pyridin-3-yl)-4-azaspiro[2.4]heptane-7-carbonitrile

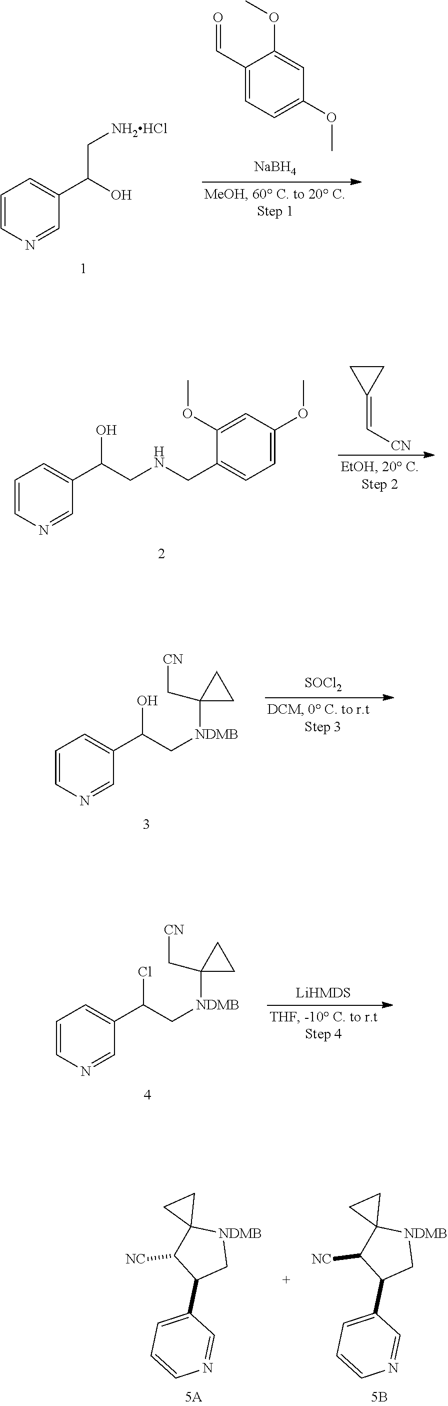

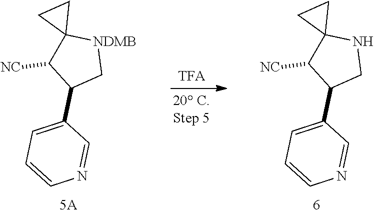

### Step 1: Preparation of 2-((2,4-dimethoxybenzyl)amino)-1-(pyridin-3-yl)ethan-1-ol

A solution of 2-amino-1-(pyridin-3-yl)ethan-1-ol dihydrochloride (1, 2.8 g, 13.2 mmol, 1 eq.) and 2,4-dimethoxybenzaldehyde (2.19 g, 13.2 mmol, 1 eq.) in MeOH (10 mL) was stirred at 60° C. for 0.5 h, then NaBH₄ (1.50 g, 39.5 mmol, 3 eq.) was added at 20° C. and the reaction mixture was stirred at 20° C. for 16 h under N₂ atmosphere. After completion, the reaction mixture was poured into ice-NH₄Cl (100 mL), then extracted with EtOAc (50 mL×3). The organic layers were combined and washed with brine (100 mL), dried over anhydrous Na₂SO₄, and concentrated under reduced pressure to afford 2-((2,4-dimethoxybenzyl)amino)-1-(pyridin-3-yl)ethan-1-ol (2, 2.50 g, 8.67 mmol, 66%) as a yellow oil. LCMS (ESI): m/z=289.2 [M+H]⁺. ¹H NMR (400 MHz, DMSO) δ 8.52 (d, J=2.0 Hz, 1H), 8.44 (dd, J=4.8, 1.6 Hz, 1H), 7.71 (dt, J=7.8, 1.8 Hz, 1H), 7.33 (dd, J=7.7, 4.9 Hz, 1H), 7.13 (d, J=8.2 Hz, 1H), 6.52 (d, J=2.3 Hz, 1H), 6.45 (dd, J=8.2, 2.4 Hz, 1H), 5.46 (s, 1H), 4.70 (t, J=6.0 Hz, 1H), 3.74 (d, J=3.2 Hz, 6H), 3.64 (d, J=4.7 Hz, 2H), 3.17 (d, J=5.1 Hz, 1H), 2.70-2.62 (m, 2H).

### Step 2: Preparation of 2-(1-((3,4-dimethylbenzyl)(2-hydroxy-2-(pyridin-3-yl)ethyl)amino)cyclopropyl)acetonitrile

To a mixture of 2-((2,4-dimethoxybenzyl)amino)-1-(pyridin-3-yl)ethan-1-ol (2, 2.5 g, 8.67 mmol, 1 eq.) in EtOH (5 mL) was added 2-cyclopropylideneacetonitrile in PE solution (1.36 g, 17.3 mmol, 2 eq.) and the resulting mixture was stirred at 20° C. for 1 h under N₂ atmosphere. After completion, the reaction mixture was concentrated under reduced pressure to give a residue. The residue was purified by flash column chromatography on silica gel to get 2-(1-((3,4-dimethylbenzyl)(2-hydroxy-2-(pyridin-3-yl)ethyl)amino)cyclopropyl)acetonitrile (3, 2.40 g, 6.53 mmol, 75%) as a pale-yellow oil. LCMS (ESI): m/z=368.2 [M+H]⁺. ¹H NMR (400 MHz, DMSO) δ 8.40 (dd, J=4.7, 1.6 Hz, 1H), 8.30 (d, J=1.8 Hz, 1H), 7.54 (dt, J=7.8, 1.8 Hz, 1H), 7.28 (dd, J=7.8, 4.8 Hz, 1H), 7.18 (d, J=8.3 Hz, 1H), 6.54 (d, J=2.3 Hz, 1H), 6.47 (dd, J=8.3, 2.4 Hz, 1H), 4.99 (d, J=3.1 Hz, 1H), 4.28 (dd, J=9.8, 6.6 Hz, 1H), 3.85-3.71 (m, 8H), 2.94-2.71 (m, 4H), 0.60-0.31 (m, 3H), 0.06 (s, 1H).

### Step 3: Preparation of 2-(1-((2-chloro-2-(pyridin-3-yl)ethyl)(3,4-dimethylbenzyl)amino)cyclopropyl)acetonitrile

SOCl₂ in DCM solution (3, 281 mg, 2.37 mmol, 1.2 eq.) was added to a solution 2-(1-((3,4-dimethylbenzyl)(2-hydroxy-2-(pyridin-3-yl)ethyl)amino)cyclopropyl)acetonitrile (3, 730 mg, 1.98 mmol, 1 eq.) in DCM (10 mL) at 0° C. The solution was stirred at room temperature for 0.5 h. After completion, the reaction mixture was quenched with NaHCO₃ (aq., sat.) (20 mL), then extracted with EtOAc (50 mL×2). The organic layers were combined and washed with brine (100 mL), dried over anhydrous Na₂SO₄, and concentrated under reduced pressure. The residue was purified by flash column chromatography on silica gel to afford 2-(1-((2-chloro-2-(pyridin-3-yl)ethyl)(3,4-dimethylbenzyl)amino)cyclopropyl)acetonitrile (4, 310 mg, 0.80 mmol, 41%) as an oil. LCMS (ESI): m/z=386.2 [M+H]⁺. ¹H NMR (400 MHz, DMSO) δ 8.50 (dd, J=4.8, 1.5 Hz, 1H), 8.31 (d, J=1.9 Hz, 1H), 7.64-7.57 (m, 1H), 7.37 (dd, J=7.9, 4.8 Hz, 1H), 7.13 (d, J=8.3 Hz, 1H), 6.55 (d, J=2.3 Hz, 1H), 6.49 (dd, J=8.3, 2.4 Hz, 1H), 4.62 (t, J=7.2 Hz, 1H), 3.81-3.72 (m, 8H), 3.36-3.23 (m, 2H), 2.91-2.75 (m, 2H), 0.61-0.38 (m, 3H), 0.06-−0.04 (m, 1H).

### Step 4: Preparation of racemic-trans-4-(3,4-dimethylbenzyl)-6-(pyridin-3-yl)-4-azaspiro[2.4]heptane-7-carbonitrile & racemic-cis-4-(3,4-dimethylbenzyl)-6-(pyridin-3-yl)-4-azaspiro[2.4]heptane-7-carbonitrile

To a solution of 2-(1-((2-chloro-2-(pyridin-3-yl)ethyl)(3,4-dimethylbenzyl)amino)cyclopropyl)acetonitrile (4, 310 mg, 0.80 mmol, 1 eq.) in THF (10 mL) was added LiHMDS (202 mg, 2.00 mmol, 2.5 eq.) dropwise at −10° C. and the solution was stirred at room temperature for 1 hr. After completion, the reaction mixture was quenched with NH₄Cl (aq., sat.) (15 mL), then extracted with EtOAc (15 mL×2). The organic layers were combined and washed with brine (10 mL), dried over anhydrous Na₂SO₄, and concentrated under reduced pressure. The residue was purified by flash column chromatography on silica gel to afford racemic-trans-4-(3,4-dimethylbenzyl)-6-(pyridin-3-yl)-4-azaspiro[2.4]heptane-7-carbonitrile (5A, 75 mg, 0.21 mmol, 27%) and cis-4-(3,4-dimethylbenzyl)-6-(pyridin-3-yl)-4-azaspiro[2.4]heptane-7-carbonitrile (5B, 55.0 mg, 0.16 mmol, 20%) as a yellow oil. LCMS (ESI): m/z=350.2 [M+H]⁺.

### Step 5: Preparation of racemic-trans-6-(pyridin-3-yl)-4-azaspiro[2.4]heptane-7-carbonitrile

A solution of racemic-trans-4-(3,4-dimethylbenzyl)-6-(pyridin-3-yl)-4-azaspiro[2.4]heptane-7-carbonitrile (5A, 100 mg, 0.29 mmol, 1 eq.) in TFA (5 mL) was stirred at 20° C. overnight under N₂. After completion, the reaction mixture was quenched with NaHCO₃ (aq., sat.) (20 mL), then extracted with EtOAc (20 mL×2). The organic layers were combined and washed with brine (100 mL), dried over anhydrous Na₂SO₄, and concentrated under reduced pressure to afford racemic-trans-6-(pyridin-3-yl)-4-azaspiro[2.4]heptane-7-carbonitrile (6, 55.0 mg, quant.) as an oil for next Step without further purification. LCMS (ESI): m/z=199.9 [M+H]⁺.

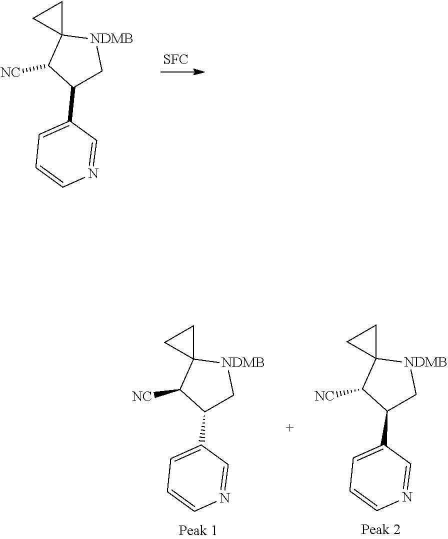

Rac-(6R,7S)-4-(2,4-dimethoxybenzyl)-6-(pyridin-3-yl)-4-azaspiro[2.4]heptane-7-carbonitrile (8 g) was separated by chiral SFC to afford (6S,7R)-4-(2,4-dimethoxybenzyl)-6-(pyridin-3-yl)-4-azaspiro[2.4]heptane-7-carbonitrile (Peak 1, 4.0 g, 50%) and (6R,7S)-4-(3,4-dimethoxybenzyl)-6-(pyridin-3-yl)-4-azaspiro[2.4]heptane-7-carbonitrile (Peak 2, 3.2 g, 40%) as a white solid. Absolute stereochemical configuration was assigned arbitrarily as drawn.

Peak 1: Retention time: 1.946 min; >99% ee.

Peak 2: Retention time: 2.456 min; >99% ee.

Analytical method: Column: ChiralPak IG, 100×4.6 mm I.D., 5 um, Mobile phase: A for CO₂ and B for ethanol (0.05% DEA), Gradient: 8 min @B 30%, Flow rate: 2.5 mL/min, Back pressure: 100 bar, Column temperature: 35° C.

SFC method: Instrument: Waters Thar 80 preparative SFC, Column: ChiralPak IG, 250×21.2 mm I.D., 5 μm, Mobile phase: A for CO₂ and B for EtOH+0.1% NH₃H₂O, Gradient: B 30%, Flow rate: 40 mL/min, Back pressure: 100 bar, Column temperature: 35° C., Wavelength: 220 nm, Cycle-time: 5.2 min, Eluted time: 3 H.

The intermediates in the table below were prepared using the method described above in Step 5 for racemic-trans-6-(pyridin-3-yl)-4-azaspiro[2.4]heptane-7-carbonitrile utilizing the appropriate starting materials and modifications.

| Name | Structure | LCMS |
|---|---|---|
| (6S,7R)-6-(pyridin-3-yl)-4-azaspiro[2.4]heptane-7- carbonitrile | 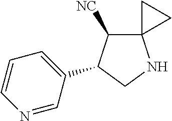 [SMILES: N#C[C@@H]1[C@@H](c2cccnc2)CNC12CC2] | 199.9 [M + H]⁺ |
| (6R,7S)-6-(pyridin-3-yl)-4-azaspiro[2.4]heptane-7- carbonitrile | 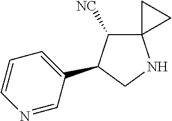 [SMILES: N#C[C@H]1[C@H](c2cccnc2)CNC12CC2] | 199.9 [M + H]⁺ |

### Synthesis of tert-butyl (R)-6-(pyridin-3-yl)-4-azaspiro[2.4]heptane-4-carboxylate and tert-butyl (S)-6-(pyridin-3-yl)-4-azaspiro[2.4]heptane-4-carboxylate

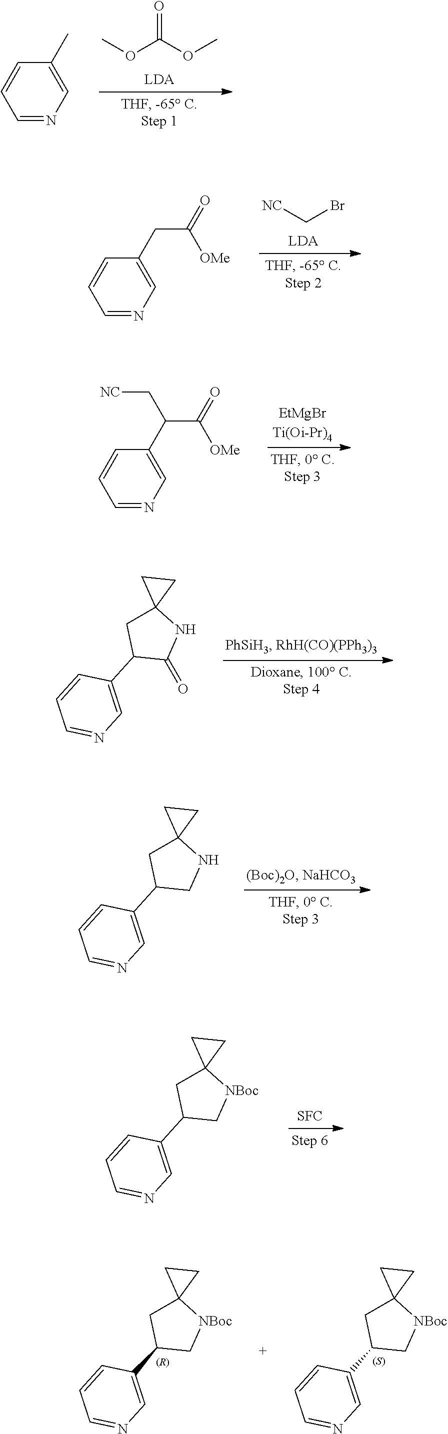

### Step 1: Preparation of methyl 2-(pyridin-3-yl)acetate

To a solution of 3-methylpyridine (1, 30 g, 322 mmol, 1.0 eq.) in THF (600 mL) was added dropwise LDA (193 mL, 386 mmol, 2M, 1.2 eq.) at −65° C. under N₂ atmosphere. The reaction solution was stirred at −65° C. for 1 h and dimethyl carbonate (57 g, 483 mmol, 1.5 eq.) was added into the reaction mixture. After addition, the mixture was stirred at −65° C. for an additional 1 hr. After completion, the reaction mixture was quenched with saturated NH₄Cl (aq.) solution (500 mL) at 0° C., then extracted with EtOAc (500 mL×2). The organic layers were combined and washed with brine (150 mL), dried over anhydrous Na₂SO₄ and concentrated under reduced pressure. The residue was purified by flash column chromatography on silica gel to afford methyl 2-(pyridin-3-yl)acetate (2, 25.0 g, 165 mmol, 51%) as a yellow oil. LCMS (ESI): m/z=152 [M+H]⁺. ¹H NMR (400 MHz, CDCl₃) δ 8.60-8.48 (m, 2H), 7.68-7.60 (m, 1H), 7.29-7.23 (m, 1H), 3.71 (s, 3H), 3.64 (s, 2H).

### Step 2: Preparation of methyl 3-cyano-2-(pyridin-3-yl)propanoate

To a solution of LDA (99.0 mL, 198 mmol, 2M, 1.5 eq.) in THF (400 mL) was added dropwise a solution of methyl 2-(pyridin-3-yl)acetate (2, 20 g, 132 mmol, 1.0 eq.) at −65° C. under N₂ atmosphere. The reaction solution was stirred at −65° C. for 1 h and 2-bromoacetonitrile (18.9 g, 158 mmol, 1.2 eq.) was added into the reaction mixture. After addition, the mixture was stirred at −65° C. for an additional 1 h. After completion, the reaction mixture was quenched with saturated ice-NH₄Cl (aq.) solution (500 mL), then extracted with EtOAc (500 mL×3). The organic layers were combined and washed with brine (500 mL), dried over anhydrous Na₂SO₄ and concentrated under reduced pressure. The residue was purified by flash column chromatography on silica gel to afford methyl 3-cyano-2-(2-methoxypyridin-4-yl)propanoate (3, 20.5 g, 107 mmol, 82%) as a yellow oil. LCMS (ESI): m/z=191 [M+H]⁺. ¹H NMR (400 MHz, CDCl₃) δ 8.62 (dd, J=4.8, 1.5 Hz, 1H), 8.57 (d, J=2.2 Hz, 1H), 7.69-7.61 (m, 1H), 7.34 (dd, J=7.9, 4.8 Hz, 1H), 4.00 (t, J=7.5 Hz, 1H), 3.75 (s, 3H), 3.07 (dd, J=16.9, 7.1 Hz, 1H), 2.88 (dd, J=16.9, 7.9 Hz, 1H).

### Step 3: Preparation of 6-(pyridin-3-yl)-4-azaspiro[2.4]heptan-5-one

To a solution of methyl 3-cyano-2-(pyridin-3-yl)propanoate (3, 20.5 g, 107 mmol, 1.0 eq.) and Titanium tetraisopropanolate (36.8 g, 128 mmol, 1.2 eq.) in THF (400 mL) was added dropwise EtMgBr (80 mL, 240 mmol, 3M, 2.25 eq.) at 0° C. After addition, the reaction mixture was stirred at 0° C. for 1 hr. After completion, the reaction mixture was quenched by addition of HCl aqueous solution (140 mL, 2N) at 0° C. The suspension was warmed to room temperature and filtered. The filtrate was poured into ice-water (500 mL) and extracted with EtOAc (200 mL×2). The organic layers were combined, washed with brine (500 mL), dried over anhydrous Na₂SO₄, and concentrated under reduced pressure. The residue was purified by flash column chromatography on silica gel to afford 6-(pyridin-3-yl)-4-azaspiro[2.4]heptan-5-one (4, 9.2 g, 48.8 mmol, 46%) as a yellow solid. LCMS (ESI): m/z=189 [M+H]⁺. ¹H NMR (400 MHz, CDCl₃) δ 8.59 (d, J=2.1 Hz, 1H), 8.53 (dd, J=4.8, 1.5 Hz, 1H), 7.74-7.65 (m, 1H), 7.29 (dd, J=8.0, 4.9 Hz, 1H), 7.00 (s, 1H), 3.91 (t, J=8.7 Hz, 1H), 2.53 (dd, J=12.9, 9.3 Hz, 1H), 2.33 (dd, J=12.9, 8.2 Hz, 1H), 1.00-0.68 (m, 4H).

### Step 4: Preparation of 6-(pyridin-3-yl)-4-azaspiro[2.4]heptane

To a solution of 6-(pyridin-3-yl)-4-azaspiro[2.4]heptan-5-one (4, 3.0 g, 15.9 mmol, 1.0 eq.) and methanidylidyneoxidanium tris(triphenylphosphane) hydrogen rhodium (729 mg, 795 mmol, 0.05 eq.) in dioxane (60 mL) was added phenylsilane (10.32 g, 95.4 mmol, 6.0 eq.). The reaction solution was stirred at 100° C. for 12 h. After completion, the reaction mixture was concentrated under reduced pressure to give a residue. The residue was triturated with 1N HCl (200 mL) at 25° C. for 0.5 h and then filtered. The filtrate was extracted with EtOAc (100 mL×2). The organic layers were combined, dried over anhydrous Na₂SO₄ and concentrated under reduced pressure. The residue was purified by Biotage® C18 column chromatography to afford 6-(pyridin-3-yl)-4-azaspiro[2.4]heptane (5, 2.2 g, 12.62 mmol, 79%) as a brown solid. LCMS (ESI): m/z=175[M+H]⁺. ¹H NMR (400 MHz, CDCl₃) δ 8.55-8.46 (m, 2H), 7.72-7.62 (m, 1H), 7.31-7.25 (m, 1H), 3.84-3.71 (m, 2H), 3.40-3.28 (m, 1H), 2.32-2.17 (m, 2H), 1.52-1.37 (m, 2H), 0.91-0.76 (m, 2H).

### Step 5: Preparation of tert-butyl 6-(pyridin-3-yl)-4-azaspiro[2.4]heptane-4-carboxylate

To a solution of 6-(pyridin-3-yl)-4-azaspiro[2.4]heptane (5, 8 g, 45.9 mmol, 1 eq.) in THF/H₂O (80 mL/10 mL) were added NaHCO₃ (7.7 g, 91.7 mmol, 2 eq.) and Boc₂O (12 g, 55.1 mmol, 1.2 eq.). The solution was stirred at room temperature for 4 h. After completion, the reaction mixture was diluted with H₂O (100 mL) and extracted with EtOAc (100 mL×3). The organic layers were combined and washed with brine (100 mL), dried over anhydrous Na₂SO₄, and concentrated under reduced pressure. The residue was purified by flash column chromatography on silica gel to afford tert-butyl 6-(pyridin-3-yl)-4-azaspiro[2.4]heptane-4-carboxylate (6, 6.7 g, 24.5 mmol, 53%) as a white solid. LCMS (ESI): m/z=275 [M+H]⁺.

### Step 6: Preparation of tert-butyl (R)-6-(pyridin-3-yl)-4-azaspiro[2.4]heptane-4-carboxylate and tert-butyl (S)-6-(pyridin-3-yl)-4-azaspiro[2.4]heptane-4-carboxylate

tert-butyl 6-(pyridin-3-yl)-4-azaspiro[2.4]heptane-4-carboxylate (6, 6.7 g) was separated by chiral SFC to afford tert-butyl (R)-6-(pyridin-3-yl)-4-azaspiro[2.4]heptane-4-carboxylate (Peak 1, 3 g, 45%) and tert-butyl (S)-6-(pyridin-3-yl)-4-azaspiro[2.4]heptane-4-carboxylate (Peak 2, 3.1 g, 46%). LCMS (ESI): m/z=275 [M+H]⁺. SFC method: Instrument: Waters Thar 80 preparative SFC Column: ChiralPak C-IG, 250×21.2 mm I.D., 5 μm Mobile phase: A for CO₂ and B for MEOH+0.1% NH₃H₂O, Gradient: B 45% Flow rate: 40 mL/min, Back pressure: 100 bar, Column temperature: 35° C., Wavelength: 220 nm Cycle-time: 25 min Eluted time: 7H. The assignment of absolute stereochemical configuration was made by comparison of experimental vibrational circular dichroism (VCD) spectra with theoretical VCD spectra obtained from DFT calculations.

### Synthesis of (R)-6-phenyl-4-azaspiro[2.4]heptane and (S)-6-phenyl-4-azaspiro[2.4]heptane

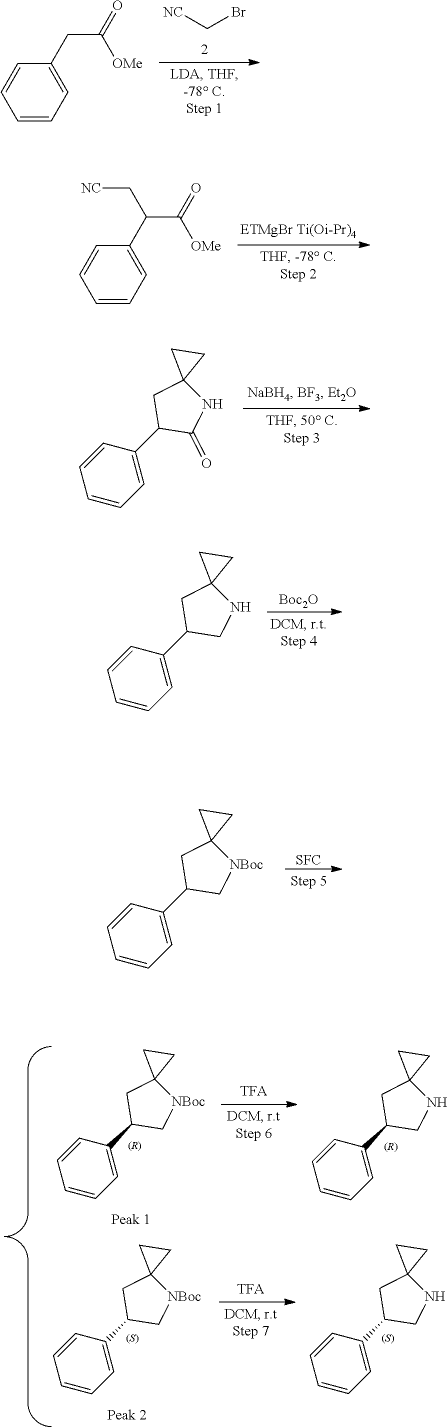

### Step 1: Preparation of methyl 3-cyano-2-phenylpropanoate

To a solution of LDA (5.95 mL, 47.8 mmol, 1.2 eq) in THF (80 mL) was added dropwise methyl 2-phenylacetate (6 g, 39.9 mmol, 1.0 eq) at −78° C. under N₂ atmosphere. The reaction solution was stirred at −78° C. for 0.5 h and 2-bromoacetonitrile (5.25 g, 43.8 mmol, 1.1 eq) was introduced into the reaction. After addition, the mixture was stirred at −78° C. for 0.5 h. After completion, the reaction mixture was quenched with saturated ice-NH₄Cl (aq.) solution (500 mL), then extracted with EtOAc (500 mL×3). The organic layers were combined and washed with brine (500 mL), dried over anhydrous Na₂SO₄ and concentrated under reduced pressure. The residue was purified by flash column chromatography on silica gel to afford methyl 3-cyano-2-phenylpropanoate (6.20 g, 32.7 mmol, 82%) as a yellow oil. LCMS (ESI): m/z=190 [M+H]⁺.

### Step 2: Preparation of 6-phenyl-4-azaspiro[2.4]heptan-5-one

To a solution of methyl 3-cyano-2-phenylpropanoate (6.2 g, 32.7 mmol, 1 eq) in THF (50 mL) was added dropwise Ti(Oi-Pr)₄ (10.2 g, 35.9 mmol, 1.1 eq) at −78° C. The reaction solution was stirred at −78° C. for 10 min and EtMgBr (72 mL, 71.9 mmol, 2.2 eq) was added dropwise into the reaction mixture. The reaction was stirred at −78° C. for 10 min then warmed up to room temperature for 0.5 h. After completion, the reaction mixture was quenched with H₂O (5 mL), followed by 10% aqueous HCl (50 mL) and EtOAc (500 mL). And 10% aq. NaOH solution was added to the resulting clear mixture until the pH was adjusted pH=8-9. The filtrate was extracted with EtOAc (200 mL×2). The organic layers were combined, washed with brine (500 mL), dried over anhydrous Na₂SO₄ and concentrated under reduced pressure. The residue was purified by flash column chromatography on silica gel to afford 6-phenyl-4-azaspiro[2.4]heptan-5-one (9.2 g, 48.8 mmol, 46%) as a yellow solid. LCMS (ESI): m/z=188.2 [M+H]⁺. ¹H NMR (400 MHz, CDCl₃) δ 7.39-7.24 (m, 6H), 3.86 (dd, J=9.4, 7.6 Hz, 1H), 2.51 (dd, J=12.9, 9.4 Hz, 1H), 2.29 (dd, J=12.9, 7.6 Hz, 1H), 0.85-0.88 (m, 1H), 0.87-0.81 (m, 1H), 0.76-0.64 (m, 2H).

### Step 3: Preparation of 6-phenyl-4-azaspiro[2.4]heptane

To a solution of 6-phenyl-4-azaspiro[2.4]heptan-5-one (2.2 g, 11.7 mmol, 1 eq) in THF (100 mL) was added NaBH₄ (2.21 g, 58.5 mmol, 5 eq) at 0° C., then Boron trifluoride etherate (8.30 g, 58.5 mmol, 5 eq) was added dropwise to the mixture. The mixture was stirred at room temperature for 5 h and then stirred at 50° C. overnight. After completion, the reaction mixture was quenched with NaHCO₃ aqueous solution (20 mL) and extracted with EtOAc (20 mL×2). The organic layers were combined, washed with brine (500 mL), dried over anhydrous Na₂SO₄ and concentrated under reduced pressure. The residue was purified by C18 column chromatography to afford 6-phenyl-4-azaspiro[2.4]heptane (1.50 g, 8.65 mmol, 74%) as an oil. LCMS (ESI): m/z=174.3 [M+H]⁺.

### Step 4: Preparation of tert-butyl 6-phenyl-4-azaspiro[2.4]heptane-4-carboxylate

To a solution of 6-phenyl-4-azaspiro[2.4]heptane (1.2 g, 6.92 mmol, 1 eq), DIEA (1.78 g, 13.8 mmol, 2 eq) and DMAP (84.5 mg, 692 μmol, 0.1 eq) in DCM (20 mL) was added di-tert-butyl dicarbonate (301 mg, 1.38 mmol, 1.2 eq) at room temperature. The reaction solution was stirred at room temperature for 5 h. After completion, the reaction mixture was concentrated under reduced pressure. The residue was purified by flash chromatography on silica gel to get tert-butyl 6-phenyl-4-azaspiro[2.4]heptane-4-carboxylate (1.50 g, 5.48 mmol, 79%) as a white solid. LCMS (ESI): m/z=218.3 [(M-tBu)+H]⁺. ¹H NMR (400 MHz, CDCl₃) δ 7.38-7.19 (m, 5H), 3.98 (s, 1H), 3.54-3.39 (m, 2H), 2.48-2.27 (m, 1H), 1.95 (dd, J=12.2, 5.5 Hz, 1H), 1.52 (s, 2H), 1.43 (s, 9H), 0.55-0.42 (m, 2H)

### Step 5: Preparation of tert-butyl (R)-6-phenyl-4-azaspiro[2.4]heptane-4-carboxylate and tert-butyl (S)-6-phenyl-4-azaspiro[2.4]heptane-4-carboxylate

Tert-butyl 6-phenyl-4-azaspiro[2.4]heptane-4-carboxylate (1.50 g, 5.48 mmol) was further separated by Chiral SFC to give: SFC Method: Instrument: SHIMADZU PREP SOLUTION SFC, Column: ChiralPak IH, 250×21.2 mm I.D., 5 μm, Mobile phase: A for CO₂ and B for MeOH+0.1% NH₃H₂O, Gradient: B 10%, Flow rate: 10 mL/min, Back pressure: 100 bar, Column temperature: 35° C., Wavelength: 220 nm, Cycle-time: 4 min, Eluted time: 4 h. Absolute stereochemistry was arbitrarily assigned.

Peak 1: tert-butyl (R)-6-phenyl-4-azaspiro[2.4]heptane-4-carboxylate or tert-butyl (S)-6-phenyl-4-azaspiro[2.4]heptane-4-carboxylate (600 mg, 40%); Retention time: 3.287 min, 99% ee. LCMS (ESI): m/z=218.3 [(M-tBu)+H]⁺. ¹H NMR (400 MHz, CDCl₃) δ 7.35-7.21 (m, 5H), 3.99 (s, 1H), 3.54-3.40 (m, 2H), 2.37 (dd, J=13.9, 8.8 Hz, 1H), 1.96 (dd, J=12.1, 5.5 Hz, 1H), 1.43 (s, 9H), 0.57-0.41 (m, 2H).

Peak 2: tert-butyl (S)-6-phenyl-4-azaspiro[2.4]heptane-4-carboxylate or tert-butyl (R)-6-phenyl-4-azaspiro[2.4]heptane-4-carboxylate (600 mg, 40%); Retention time: 3.582 min, 97% ee. LCMS (ESI): m/z=218.3 [(M-tBu)+H]⁺. ¹H NMR (400 MHz, CDCl₃) δ 7.37-7.20 (m, 5H), 3.99 (s, 1H), 3.56-3.38 (m, 2H), 2.37 (dd, J=13.8, 8.8 Hz, 1H), 1.96 (dd, J=12.1, 5.5 Hz, 1H), 1.43 (s, 9H), 0.58-0.42 (m, 2H).

Analytical method: Column: (R,R)-Whelk-O1, 250×4.6 mm I.D., 5 um, Mobile phase: A for CO₂ and B for IPA (0.05% DEA), Gradient: 10 min @B 20%, Flow rate: 2.0 mL/min, Back pressure: 100 bar, Column temperature: 35° C.

### Step 6: Preparation of (R)-6-phenyl-4-azaspiro[2.4]heptane or (S)-6-phenyl-4-azaspiro[2.4]heptane

To the solution of tert-butyl (R)-6-phenyl-4-azaspiro[2.4]heptane-4-carboxylate or tert-butyl (S)-6-phenyl-4-azaspiro[2.4]heptane-4-carboxylate (60 mg, 219 μmol, 1 eq, Peak 1) in DCM (2 mL) was added TFA (1 mL) at room temperature. The solution was stirred at room temperature. for 2 h. After completion, the reaction mixture was concentrated under reduced pressure to give (R)-6-phenyl-4-azaspiro[2.4]heptane or (S)-6-phenyl-4-azaspiro[2.4]heptane (70.0 mg, quant.) as an oil, which was used in next Step directly without further purification. LCMS (ESI): m/z=174.2 [M+H]⁺.

### Step 7: Preparation of (S)-6-phenyl-4-azaspiro[2.4]heptane or (R)-6-phenyl-4-azaspiro[2.4]heptane

To the solution of tert-butyl (6S)-6-phenyl-4-azaspiro[2.4]heptane-4-carboxylate or tert-butyl (R)-6-phenyl-4-azaspiro[2.4]heptane-4-carboxylate (60 mg, 219 μmol, 1 eq, Peak 2) in DCM (2 mL) was added TFA (1 mL) at room temperature. The solution was stirred at room temperature. for 2 h. After completion, the reaction mixture was concentrated under reduced pressure to give (S)-6-phenyl-4-azaspiro[2.4]heptane or (R)-6-phenyl-4-azaspiro[2.4]heptane (70.0 mg, quant.) as an oil, which was used in next Step directly without further purification. LCMS (ESI): m/z=174.2 [M+H]⁺.

### Preparation of N,N-dimethyl-3-(4-azaspiro[2.4]heptan-6-yl)pyridin-4-amine

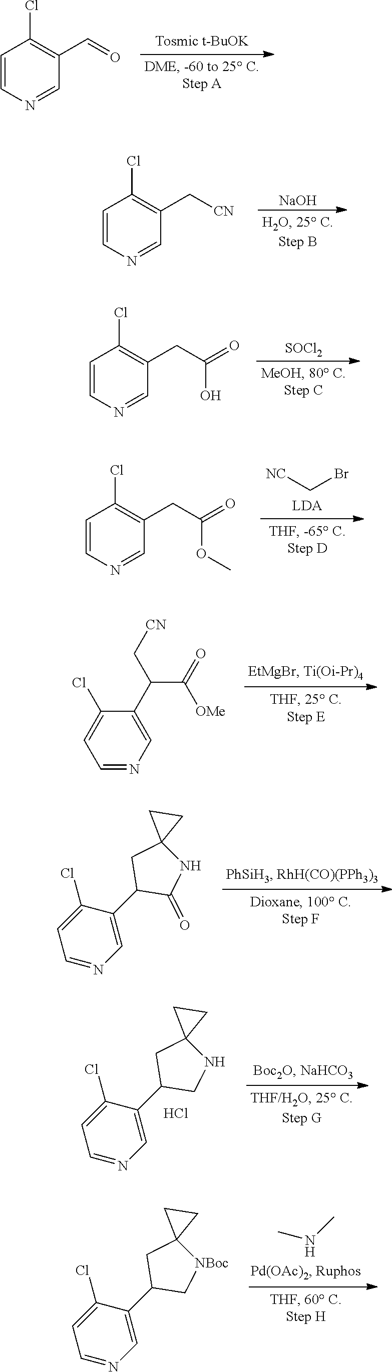

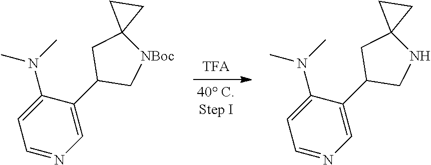 [SMILES: CN(C)c1ccncc1C1CN(C(=O)OC(C)(C)C)C2(CC2)C1 ; CN(C)c1ccncc1C1CNC2(CC2)C1]

### Step A: 2-(4-chloropyridin-3-yl)acetonitrile

To a solution of t-BuOK (15.8 g, 141 mmol, 2 eq.) in DME (100 mL) was added TosMIC (16.5 g, 84.7 mmol, 1.2 eq.) at 25° C. The reaction mixture was cooled to −60° C. 4-chloronicotinaldehyde (10 g, 70.6 mmol, 1 eq.) in DME (100 mL) was added dropwise to the mixture at −60° C. The reaction was stirred at −60° C. for 1 hr, then warmed up to 25° C. and stirred for 2 hrs under N₂ atmosphere. MeOH (100 mL) was added to the mixture, and the reaction mixture was heated to 80° C. and stirred at 80° C. for 0.5 hrs under N₂ atmosphere. After completion, the reaction mixture was quenched by adding H₂O (200 mL), then extracted with EtOAc (100 mL×3). The organic layers were combined and washed with brine (150 mL), dried over anhydrous Na₂SO₄, and concentrated under reduced pressure. The residue was purified by flash column chromatography on silica gel to afford 2-(4-chloropyridin-3-yl)acetonitrile (4.2 g, 27.5 mmol, 39%) as a yellow solid. LC-MS (ESI) m/z=153 [M+H]⁺.

### Step B: 2-(4-chloropyridin-3-yl)acetic acid

2-(4-chloropyridin-3-yl)acetonitrile (4.2 g, 27.5 mmol, 1.0 eq.) was added to a solution of sodium hydroxide (9 g, 225 mmol, 8.2 eq.) in H₂O (51 mL) at 25° C. The reaction mixture was heated to 100° C. and stirred for 1 hr. After completion, the reaction mixture was cooled down in an ice bath, then acidified carefully with HCl (con.) until the pH was adjusted to pH=1. The resulting mixture was dissolved in MeOH (50 mL). The suspension was filtered through a pad of Celite, the filter cake was washed with MeOH (10 mL). The combined filtrates were concentrated under reduced pressure to give 2-(4-chloropyridin-3-yl)acetic acid (4.0 g, 23.3 mmol, 85%) as a brown solid. LC-MS (ESI) m/z=172 [M+H]⁺.

### Step C: methyl 2-(4-chloropyridin-3-yl)acetate

To a solution of 2-(4-chloropyridin-3-yl)acetic acid (4 g, 23.3 mmol, 1 eq.) in MeOH (50 mL) was added dropwise thionyl chloride (13.7 g, 116 mmol, 5 eq.) at 25° C. The reaction mixture was heated to 80° C. and stirred for 1 hr. After completion, the reaction mixture was concentrated under reduce pressure. The product was dissolved in water (100 mL), the aqueous phase was neutralized carefully with NaHCO₃ (aq.) until the pH was adjusted to pH=9. The resulting mixture was extracted with EtOAc (100 mL×2), and the combined organic layers were washed with brine (100 mL), dried over with anhydrous Na₂SO₄, then concentrated under reduced pressure. The residue was purified by flash column chromatography on silica gel to afford methyl 2-(4-chloropyridin-3-yl)acetate (4.0 g, 21.5 mmol, 93%) as a yellow oil. LCMS (ESI): m/z=186 [M+H]⁺.

### Step D: methyl 2-(4-chloropyridin-3-yl)-3-cyanopropanoate

To a solution of methyl 2-(4-chloropyridin-3-yl)acetate (3 g, 16.1 mmol, 1 eq.) in dry THF (10 mL) was added LDA (9.7 mL, 19.3 mmol, 1.2 eq.) slowly at −65° C. under nitrogen. The mixture was stirred for additional 1 hr at −65° C., then 2-bromoacetonitrile (2.31 g, 19.3 mmol, 1.2 eq.) was added drop-wisely, after addition, the reaction mixture was stirred at −65° C. for 1 hr. After completion, the reaction mixture was quenched by adding sat. NH₄Cl (100 mL), then extracted with EtOAc (100 mL×2). The organic layers were combined and washed with brine (100 mL), dried over anhydrous Na₂SO₄, and concentrated under reduced pressure. The residue was purified by flash column chromatography on silica gel to afford methyl 2-(4-chloropyridin-3-yl)-3-cyanopropanoate (3.0 g, 13.3 mmol, 83%) as a yellow oil. LC-MS (ESI) m/z=225 [M+H]⁺

### Step E: 6-(4-chloropyridin-3-yl)-4-azaspiro[2.4]heptan-5-one

To a solution of methyl 2-(4-chloropyridin-3-yl)-3-cyanopropanoate (1 g, 4.45 mmol, 1 eq.) and titanium isopropoxide (1.58 g, 5.56 mmol, 1.25 eq.) in THF (30 mL) was added dropwise EtMgBr (3.7 mL, 11.1 mmol, 2.5 eq.) at 25° C. for 2 hrs. The reaction mixture was stirred at 25° C. for 0.5 hrs under N₂ atmosphere. After completion, the reaction mixture was diluted with H₂O (10 mL), then extracted with EtOAc (20 mL×3). The organic layers were combined and washed with brine (150 mL), dried over anhydrous Na₂SO₄, and concentrated under reduced pressure. The residue was purified by flash column chromatography on silica gel to afford 6-(4-chloropyridin-3-yl)-4-azaspiro[2.4]heptan-5-one (500 mg, 2.25 mmol, 51%) as a yellow oil. LC-MS (ESI) m/z=223 [M+H]⁺

### Step F: 6-(4-chloropyridin-3-yl)-4-azaspiro[2.4]heptane hydrochloride

To a solution of 6-(4-chloropyridin-3-yl)-4-azaspiro[2.4]heptan-5-one (500 mg, 2.24 mmol, 1.0 eq.) in dioxane (10 mL) were added lambda1-rhodium(1+) formyl radical tris(triphenylphosphine) hydride (103 mg, 112 μmol, 0.05 eq.) and phenylsilane (1.45 g, 13.4 mmol, 6 eq.) at 25° C. The reaction mixture was heated to 100° C. and stirred for 12 hrs under N₂ atmosphere. After completion, the reaction mixture was cooled to 25° C. and acidified carefully with HCl/dioxane (4 M) until the pH was adjusted to pH=1. The resulting mixture was concentrated under reduce pressure. The residue was triturated with H₂O (10 mL) and filtered. The filtrate was concentrate under reduce pressure to give 6-(4-chloropyridin-3-yl)-4-azaspiro[2.4]heptane hydrochloride (200 mg, 815 μmol, 36%) as a yellow solid. LC-MS (ESI) m/z=209 [M+H]⁺.

### Step G: tert-butyl 6-(4-chloropyridin-3-yl)-4-azaspiro[2.4]heptane-4-carboxylate

To a solution of 6-(4-chloropyridin-3-yl)-4-azaspiro[2.4]heptane hydrochloride (200 mg, 958 μmol, 1 eq.) and sodium bicarbonate (402 mg, 4.79 mmol, 5 eq.) in a mixture of H₂O (5 mL) and THF (5 mL) was added di-tert-butyl dicarbonate (312 mg, 1.43 mmol, 1.5 eq.) at 25° C. The reaction mixture was stirred at 25° C. for 2 hrs. After completion, the reaction mixture was diluted with H₂O (10 mL), then extracted with EtOAc (20 mL×3). The organic layers were combined and washed with brine (15 mL), dried over anhydrous Na₂SO₄, and concentrated under reduced pressure. The residue was purified by flash column chromatography on silica gel to afford tert-butyl 6-(4-chloropyridin-3-yl)-4-azaspiro[2.4]heptane-4-carboxylate (200 mg, 647 μmol, 68%) as a yellow oil. LC-MS (ESI) m/z=309 [M+H]⁺.

### Step H: tert-butyl 6-(4-(dimethylamino)pyridin-3-yl)-4-azaspiro[2.4]heptane-4-carboxylate

To a mixture of tert-butyl 6-(4-chloropyridin-3-yl)-4-azaspiro[2.4]heptane-4-carboxylate (100 mg, 0.32 mmol, 1 eq.), dimethylamine (0.64 mmol, 2.0 eq., 1 M in THF), and t-BuONa (61 mg, 0.64 mmol, 2.0 eq.) in THF (2 mL) were added Pd(OAc)₂ (5 mg, 32 μmol, 0.1 eq.) and Ruphos (30 mg, 64 μmol, 0.2 eq.). The suspension was degassed under vacuum and purged with N₂ several times. The resulting mixture was stirred at 60° C. for 0.5 hrs. After completion, the mixture was concentrated under reduced pressure. The residue was purified by flash column chromatography on silica gel to give tert-butyl 6-(4-(dimethylamino)pyridin-3-yl)-4-azaspiro[2.4]heptane-4-carboxylate (70 mg, 0.22 mmol, 69%) as a yellow oil. LCMS (ESI): m/z=318 [M+H]⁺.

### Step I: N,N-dimethyl-3-(4-azaspiro[2.4]heptan-6-yl)pyridin-4-amine

A solution of tert-butyl 6-(4-(dimethylamino)pyridin-3-yl)-4-azaspiro[2.4]heptane-4-carboxylate (100 mg, 0.31 mmol, 1.0 eq.) in TFA (0.5 mL) was stirred at 40° C. for 6 hrs. After completion, the reaction mixture was concentrated under reduced pressure to afford N,N-dimethyl-3-(4-azaspiro[2.4]heptan-6-yl)pyridin-4-amine (100 mg, quant.) (TFA salt) as a brown solid, which was used without further purification. LCMS (ESI): m/z=218 [M+H]⁺.

### Representative Procedures for Syntheses of Phosphoryl Groups:

### Synthesis of 5-((diethoxyphosphoryl)difluoromethyl)benzo[b]thiophene-2-carboxylic acid

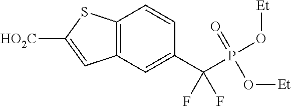 [SMILES: CCOP(=O)(OCC)C(F)(F)c1ccc2sc(C(=O)O)cc2c1]

5-((diethoxyphosphoryl)difluoromethyl)benzo[b]thiophene-2-carboxylic acid was prepared according to the protocol described in WO 2020/205467 (e.g., paragraphs [0560-0565], which are incorporated herein).

### Synthesis of (difluoro(2-((4-nitrophenoxy)carbonyl)benzo[b]thiophen-5-yl)methyl)phosphonic acid

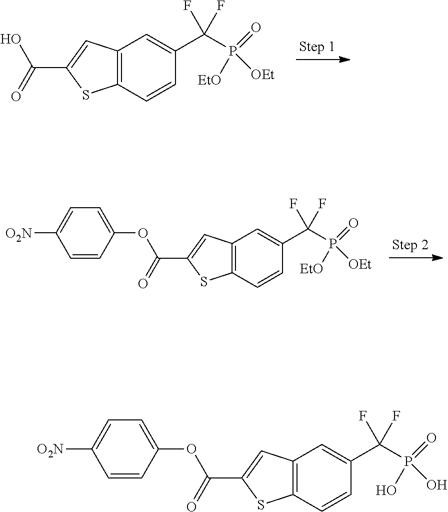 [SMILES: CCOP(=O)(OCC)C(F)(F)c1ccc2sc(C(=O)O)cc2c1 ; CCOP(=O)(OCC)C(F)(F)c1ccc2sc(C(=O)Oc3ccc([N+](=O)[O-])cc3)cc2c1 ; O=C(Oc1ccc([N+](=O)[O-])cc1)c1cc2cc(C(F)(F)P(=O)(O)O)ccc2s1]

### Step 1: Preparation of 4-nitrophenyl 5-((diethoxyphosphoryl)difluoromethyl)benzo[b]thiophene-2-carboxylate

To a mixture of 5-[(diethoxyphosphoryl)difluoromethyl]-1-benzothiophene-2-carboxylic acid (10.0 g, 27.4 mmol), EDCI (7.85 g, 41.0 mmol) and DMAP (836 mg, 6.85 mmol) in CH₂Cl₂ (80 mL) was stirred at room temperature. After 15 min, 4-nitrophenol (4.75 g, 34.2 mmol) was added and the resulting yellow mixture was stirred at room temperature for 18 h. The reaction was quenched with water (30 mL) and the product was extracted with CH₂Cl₂ (10 mL×2). The combined organic extracts were washed with brine, dried with sodium sulfate, filtered and concentrated in vacuo. The crude residue was purified by reverse phase chromatography [C18 cartridge eluting with a gradient of 5-100% acetonitrile in water] and the appropriate fractions were concentrated to give 4-nitrophenyl 5-((diethoxyphosphoryl)difluoromethyl)benzo[b]thiophene-2-carboxylate (7.80 g, 16.0 mmol, 59.0% yield) as a yellow solid. LCMS m/z=486.2 [M+H]⁺; ¹H NMR (400 MHz, DMSO-d₆) δ 8.32-8.37 (m, 3H), 8.22 (s, 1H), 8.01 (d, J=9.1 Hz, 1H), 7.77 (d, J=7.8 Hz, 1H), 7.51-7.45 (m, 2H), 4.14-4.32 (m, 4H), 1.34 (t, J=7.8 Hz, 6H).

### Step 2: Preparation of (difluoro(2-((4-nitrophenoxy)carbonyl)benzo[b]thiophen-5-yl)methyl)phosphonic acid

To a cooled (0° C.) solution of 4-nitrophenyl 5-((diethoxyphosphoryl)difluoromethyl)benzo[b]thiophene-2-carboxylate (4.47 g, 9.20 mmol) in CH₂Cl₂ (39 mL) was added N,O-bis(trimethylsilyl)trifluoroacetamide (12.1 mL, 46.0 mmol) and iodotrimethylsilane (5.23 mL, 36.8 mmol) as a solution in CH₂Cl₂ (10 mL). The reaction mixture was gradually allowed to warm to ambient temperatures. To the reaction mixture was added a mixture of 2:1 H₂O/acetonitrile (with 0.1% TFA) (50 mL) and precipitation of product was observed. The volatiles were removed in vacuo and the crude residue was suspended in a mixture of acetonitrile/water solution (1:1 v/v, 100 mL). The suspension was filtered, the solids were washed with a 2:1 mixture acetonitrile/water solution, and the solid were dried under reduced pressure to afford [difluoro({2-[(4-nitrophenoxy)carbonyl]-1-benzothiophen-5-yl})methyl]phosphonic acid (6.5 g, 94%) as a beige solid. The filtrate was concentrated to 50% of solvent volume and the resulting suspension was filtered and washed with 1:2 acetonitrile/water solution. The solid was dried under reduced pressure to afford additional [difluoro({2-[(4-nitrophenoxy)carbonyl]-1-benzothiophen-5-yl})methyl]phosphonic acid (0.4 g) as a beige solid. Both products were lyophilized to give (difluoro(2-((4-nitrophenoxy)carbonyl)benzo[b]thiophen-5-yl)methyl)phosphonic acid (6.90 g, 16.0 mmol, 98.0% yield). ¹H NMR (400 MHz, DMSO-d₆) δ 7.66-7.74 (m, 3H), 8.27 (d, J=8.3 Hz, 1H), 8.30 (s, 1H), 8.36-8.41 (m, 2H), 8.66 (s, 1H).

The intermediates in the table below was prepared using a similar protocol outlined above for synthesis of [difluoro({2-[(4-nitrophenoxy)carbonyl]-1-benzothiophen-5-yl})methyl]phosphonic acid and utilizing the appropriate intermediate(s) as starting material(s).

| Name | Structure | LC-MS |
|---|---|---|
| (difluoro(2- ((perfluorophenoxy)carbonyl)benzo [b]thiophen-5-yl)methyl)phosphonic acid | 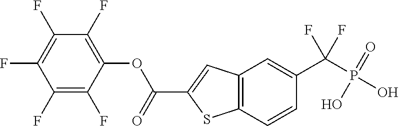 [SMILES: O=C(Oc1c(F)c(F)c(F)c(F)c1F)c1cc2cc(C(F)(F)P(=O)(O)O)ccc2s1] | 475.0 [M + H]⁺ |
| (R)-(fluoro(2- ((perfluorophenoxy)carbonyl)benzo [b]thiophen-5-yl)methyl)phosphonic acid | 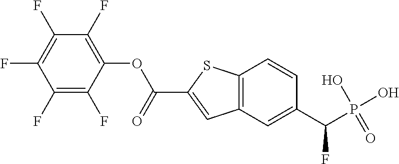 [SMILES: O=C(Oc1c(F)c(F)c(F)c(F)c1F)c1cc2cc([C@H](F)P(=O)(O)O)ccc2s1] | 457 [M + H]⁺ |
| (S)-(fluoro(2- ((perfluorophenoxy)carbonyl)benzo [b]thiophen-5-yl)methyl)phosphonic acid | 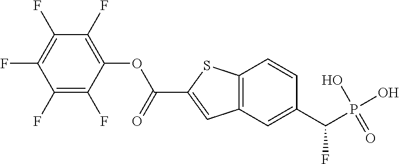 [SMILES: O=C(Oc1c(F)c(F)c(F)c(F)c1F)c1cc2cc([C@@H](F)P(=O)(O)O)ccc2s1] | 457 [M + H]⁺ |

### Synthesis of ((2-((perfluorophenoxy)carbonyl)benzo[b]thiophen-5-yl)methyl)phosphonic acid

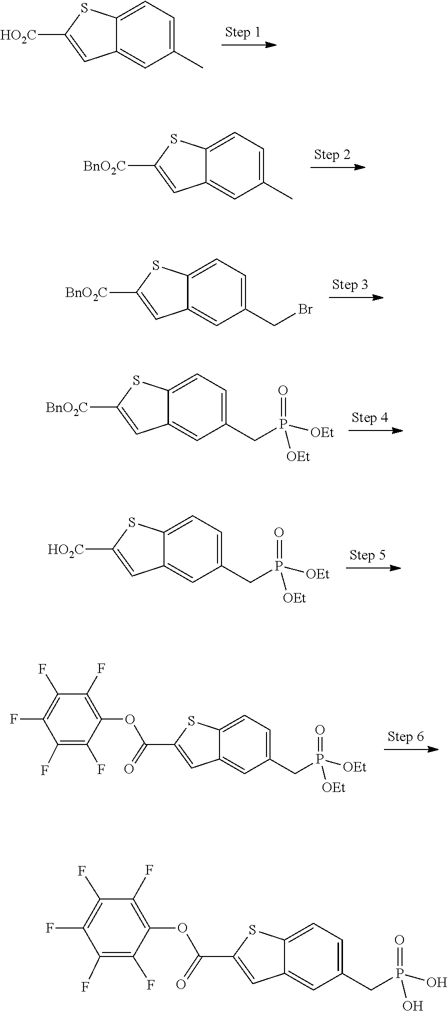

### Preparation of 5-methylbenzo[b]thiophene-2-carboxylic acid

5-Methylbenzo[b]thiophene-2-carboxylic acid was prepared according to the procedure described in WO 2016/100184 (e.g., paragraphs [0281]-[0284], which are incorporated by reference herein).

### Step 1: Preparation of benzyl 5-methylbenzo[b]thiophene-2-carboxylate

To a solution of 5-methylbenzo[b]thiophene-2-carboxylic acid (21.2 g, 110.0 mmol, 1.0 eq) and K₂CO₃ (30.4 g, 220.0 mmol, 2.0 eq) in DMF (200 mL) was added benzyl bromide (20.6 g, 121.0 mmol, 1.1 eq). The mixture was stirred at room temperature for 14 h. The reaction mixture was poured into ice water (400 mL) and stirred for 5 min. The resulting solids were filtered, and the filter cake was washed with water (50 mL), dried in vacuum to give benzyl 5-methylbenzo[b]thiophene-2-carboxylate (30.1 g, 106.0 mmol, 97% yield) as a yellow solid. ¹H NMR (400 MHz, CDCl₃) δ 8.01 (s, 1H), 7.71 (t, J=12.2 Hz, 1H), 7.65 (s, 1H), 7.46 (d, J=6.8 Hz, 2H), 7.42-7.35 (m, 3H), 7.29-7.26 (m, 1H), 5.38 (s, 2H), 2.47 (s, 3H).

### Step 2: Preparation of benzyl 5-(bromomethyl)benzo[b]thiophene-2-carboxylate

To a solution of benzyl 5-methylbenzo[b]thiophene-2-carboxylate (15.0 g, 53.1 mmol, 1.0 eq) and NBS (10.3 g, 58.4 mmol, 1.1 eq) in CCl₄ (30 mL) was added benzoyl peroxide (1.3 g, 5.31 mmol, 0.1 eq). The reaction flask was subjected to three cycles of evacuation and backfilling with N₂ (g). The mixture was stirred at 80° C. for 16 h under constant atmosphere of N₂ (g). The reaction mixture was concentrated under reduced pressure. The residue was purified by flash column chromatography on silica gel to give benzyl 5-(bromomethyl)benzo[b]thiophene-2-carboxylate (6.80 g, 18.8 mmol, 36% yield) as a yellow solid.

### Step 3: Preparation of benzyl 5-((diethoxyphosphoryl)methyl)benzo[b]thiophene-2-carboxylate

A solution of benzyl 5-(bromomethyl)benzo[b]thiophene-2-carboxylate (10.3 g, 28.5 mmol, 1.0 eq) dissolved in triethyl phosphite (30.0 g, 180.0 mmol, 6.3 eq) was stirred at 100° C. for 5 h. The reaction mixture was concentrated under reduced pressure directly, the residue was purified by flash column chromatography on silica gel to give benzyl 5-((diethoxyphosphoryl)methyl)benzo[b]thiophene-2-carboxylate (6.5 g, 15.5 mmol, 55% yield) as a colorless oil. LCMS (ESI) m/z=419 [M+H]⁺; ¹H NMR (400 MHz, CDCl₃) δ 8.04 (s, 1H), 7.80 (d, J=8.3 Hz, 2H), 7.49-7.33 (m, 6H), 5.39 (s, 2H), 4.08-3.93 (m, 4H), 3.26 (d, J=21.5 Hz, 2H), 1.24 (t, J=7.1 Hz, 6H).

### Step 4: Preparation of 5-((diethoxyphosphoryl)methyl)benzo[b]thiophene-2-carboxylic acid

To a solution of benzyl 5-((diethoxyphosphoryl)methyl)benzo[b]thiophene-2-carboxylate (5.6 g, 13.3 mmol, 1.0 eq) in dissolved in a mixture of THF (80 mL) and H₂O (10 mL) was added LiOH (1.10 g, 26.6 mmol, 2.0 eq). The mixture was stirred at room temperature for 3 h and subsequently acidified with aqueous solution of 1 N HCl (adjusted to pH ˜3-4). The product precipitated out of solution upon acidification. The resulting solids were filtered, the filter cake was washed with water (20 mL×2), and the solids were dried under vacuum to give 5-((diethoxyphosphoryl)methyl)benzo[b]thiophene-2-carboxylic acid (3.9 g, 11.8 mmol, 88.9% yield) as a white solid. LCMS (ESI) m/z=329 [M+H]⁺.

### Step 5: Preparation of perfluorophenyl 5-((diethoxyphosphoryl)methyl)benzo[b]thiophene-2-carboxylate

To a cooled (0° C.) solution of 5-((diethoxyphosphoryl)methyl)benzo[b]thiophene-2-carboxylic acid (3.9 g, 11.8 mmol, 1.0 eq) in CH₂Cl₂ (50 mL) was added oxalyl chloride (2.2 g, 17.7 mmol, 1.5 eq) followed by addition of two drops of DMF. The mixture was stirred at 0° C. for 30 min, followed by evaporation of the reaction mixture to dryness. The resulting solids were dissolved in CH₂Cl₂ (50 mL), followed by addition of Et₃N (3.6 g, 35.4 mmol, 3.0 eq) and pentafluorophenol (2.6 g, 14.1 mmol, 1.2 eq). The resulting mixture was stirred at room temperature for additional 2 h and subsequently, poured over H₂O (30 mL). The bi-phasic solution was extracted with EtOAc (30 mL×3). The combined organic layers were dried over MgSO₄, filtered and concentrated under reduced pressure, the residue was purified by column chromatography on silica gel to give perfluorophenyl 5-((diethoxyphosphoryl)methyl)benzo[b]thiophene-2-carboxylate (4.7 g, 9.5 mmol, 81% yield) as a white solid. LCMS (ESI) m/z=419 [M+H]⁺; ¹H NMR (400 MHz, CDCl₃) δ 8.29 (s, 1H), 7.88 (d, J=9.1 Hz, 2H), 7.51 (d, J=8.4 Hz, 1H), 4.14-3.94 (m, 4H), 3.29 (d, J=21.5 Hz, 2H), 1.26 (t, J=7.0 Hz, 6H).

### Step 6: Preparation of ((2-((perfluorophenoxy)carbonyl)benzo[b]thiophen-5-yl)methyl)phosphonic acid

To a solution of perfluorophenyl 5-((diethoxyphosphoryl)methyl)benzo[b]thiophene-2-carboxylate (4.7 g, 9.5 mmol, 1.0 eq) in CH₂Cl₂ (60 mL) was added bromotrimethylsilane (12 mL). The mixture was stirred at room temperature for 14 h and subsequently concentrated under reduced pressure. The residue was purified by C18 column chromatography to give ((2-((perfluorophenoxy)carbonyl)benzo[b]thiophen-5-yl)methyl)phosphonic acid (3.7 g, 8.4 mmol, 89% yield) as a white solid. LCMS (ESI) m/z=439 [M+H]⁺.

### Synthesis of (R)- or (S)-5-((diethoxyphosphoryl)fluoromethyl)benzo[b]thiophene-2-carboxylic acid and (S)-5-((diethoxyphosphoryl)fluoromethyl)benzo[b]thiophene-2-carboxylic acid

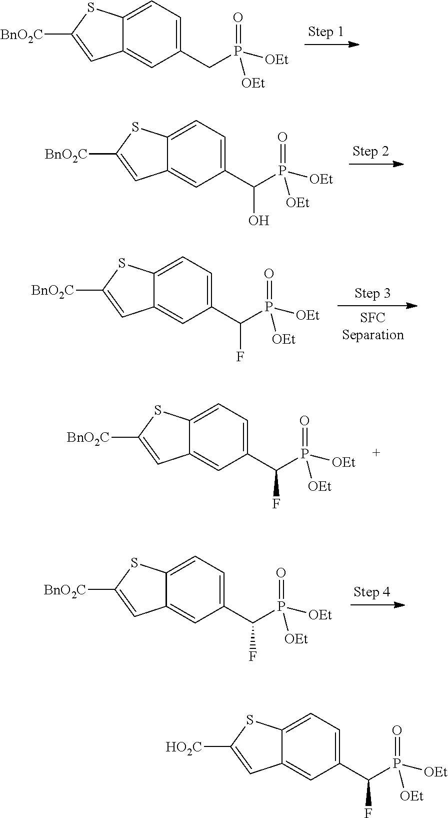

### Step 1: rac-benzyl 5-((diethoxyphosphoryl)(hydroxy)methyl)benzo[b]thiophene-2-carboxylate

To a cooled (−78° C.) solution of benzyl 5-((diethoxyphosphoryl)methyl)benzo[b]thiophene-2-carboxylate (2.4 g, 5.73 mmol, 1 eq) in THF (75 mL) and 2-(benzenesulfonyl)-3-phenyloxaziridine (2.97 g, 11.4 mmol, 2 eq) was added a 1 M solution of NaHMDS (11.4 mL, 11.4 mmol, 2 eq) in THF. A deep purple solution was observed upon addition of base that changed to orange after complete addition of the base. The mixture was stirred for an additional 10 min, followed by addition of aqueous saturated NH₄Cl (50 mL). The mixture was warmed to ambient temperatures and EtOAc (75 mL) and water (25 mL) was added. After stirring for an additional 30 min, the phases were separated. The aqueous layer was extracted with EtOAc (125 mL×2). The combined organic extracts were dried with anhydrous sodium sulfate, filtered, concentrated under reduced pressure. Another batch of equal scale was performed and combined for purification. The combined material (6.42 mmol, 12.15 mmol in total) was purified by flash chromatography (20%-100%=EtOAc:heptane) to give rac-benzyl 5-((diethoxyphosphoryl)(hydroxy)methyl)benzo[b]thiophene-2-carboxylate (3.69 g, 8.49 mmol, 70%) as a white sticky solid. LCMS (ESI) m/z=869.4 [2M+H]⁺; ¹H NMR (400 MHz, CDCl₃) δ 8.10 (s, 1H), 8.04-8.00 (m, 1H), 7.88 (d, J=8.6 Hz, 1H), 7.61 (d, J=8.6 Hz, 1H), 7.51-7.47 (m, 2H), 7.46-7.35 (m, 3H), 5.42 (s, 2H), 5.17 (dd, J=10.4, 4.5 Hz, 1H), 4.18-3.95 (m, 4H), 3.10-2.99 (m, 1H), 1.33-1.20 (m, 6H).

### Step 2: rac-benzyl 5-((diethoxyphosphoryl)fluoromethyl)benzo[b]thiophene-2-carboxylate

To a cooled (−78° C.) solution (under N₂ (g)) of rac-benzyl 5-((diethoxyphosphoryl)(hydroxy)methyl)benzo[b]thiophene-2-carboxylate (cc) (1.56 g, 3.59 mmol, 1 eq) in CH₂Cl₂ (30 mL) was added (diethylamino)sulfur trifluoride (568 L, 4.30 mmol, 1.2 eq). The reaction was stirred for 15 min, followed by addition of aqueous saturated NaHCO₃ (50 mL). After warming to room temperature, the product was extracted with CH₂Cl₂ (50 mL×3). The combined organic layers were dried over sodium sulfate, filtered and concentrated under reduced pressure. The crude residue was purified by reverse phase chromatography on C18 cartridge (eluting with 5-80% acetonitrile in water) to give rac-benzyl 5-((diethoxyphosphoryl)fluoromethyl)benzo[b]thiophene-2-carboxylate (650 mg, 1.48 mmol, 41.6%) as a thick clear oil. LCMS (ESI) m/z=873.2 [2M+H]⁺; ¹H NMR (400 MHz, CDCl₃) δ 8.12 (s, 1H), 8.02-7.99 (m, 1H), 7.92 (d, J=8.7 Hz, 1H), 7.61 (d, J=8.7 Hz, 1H), 7.51-7.51 (m, 2H), 7.46-7.36 (m, 3H), 5.82 (dd, J=44.4, 7.5 Hz, 1H), 5.42 (s, 2H), 4.21-4.02 (m, 4H), 1.34-1.26 (m, 6H).

### Step 3: Preparation of benzyl (R)-5-((diethoxyphosphoryl)fluoromethyl)benzo[b]thiophene-2-carboxylate and benzyl (S)-5-((diethoxyphosphoryl)fluoromethyl)benzo[b]thiophene-2-carboxylate

rac-Benzyl 5-((diethoxyphosphoryl)fluoromethyl)benzo[b]thiophene-2-carboxylate (650 mg, 1.48 mmol) was submitted to chiral SFC separation (Column: Lux i-Amylose 3, 21.2×250 mm 5 um column, 75 mL/min, 40% MeOH) to give benzyl (R)- or (S)-5-((diethoxyphosphoryl)fluoromethyl)benzo[b]thiophene-2-carboxylate (304 mg, 0.70 mmol, 46.8% recovery, 99.9% ee) as a thick clear oil (Peak 1) and benzyl (R)- or (S)-5-((diethoxyphosphoryl)fluoromethyl)benzo[b]thiophene-2-carboxylate (317 mg, 0.73 mmol, 49% recovery, 99.9% ee) as a thick clear oil (Peak 2). Note: Fastest eluting enantiomer by SFC was arbitrarily assigned as (R)-5-(fluoro(phosphono)methyl)benzo[b]thiophene-2-carboxylic acid and slowest eluting enantiomer by SFC as (S)-5-(fluoro(phosphono)methyl)benzo[b]thiophene-2-carboxylic acid. HPLC method for analysis of enantiomeric excess: Lux Cellulose-3 150 mm 45% H₂O+0.05% TFA/55% MeCN 1 mL/min 8 min.

### Step 4: Preparation of (R)- or (S)-5-((diethoxyphosphoryl)fluoromethyl)benzo[b]thiophene-2-carboxylic acid

To a mixture of 10% Pd/C (60 mg, 50% wet) and benzyl (R)-5-((diethoxyphosphoryl)fluoromethyl)benzo[b]thiophene-2-carboxylate (Peak 1) (60 mg, 0.1374 mmol, 1 eq) in THF (5 mL) was degassed with N₂ (g) for 5 min. To the mixture was bubbled H₂ (g) for 5 min then the reaction was allowed to stir at room temperature under H₂ (g) (1 atm). The reaction mixture was stirred until consumption of starting material was detected by LCMS. The reaction mixture was subsequently sparged N₂ (g) for 15 min and filtered over a pad of Celite®. The filter cake was washed with 2-MeTHF and the filtrate was concentrated to give (R)- or (S)-5-((diethoxyphosphoryl)fluoromethyl)benzo[b]thiophene-2-carboxylic acid (47.4 mg, 0.0137 mmol, 99%) as a thick clear oil. LCMS (ESI) m/z=347.2 [M+H]⁺.

The intermediates in the table below was prepared using the procedure outlined above (in Step 4) starting from benzyl (S)- or (R)-5-((diethoxyphosphoryl)fluoromethyl)benzo[b]thiophene-2-carboxylate (Peak 2) and using the appropriate reagents.

| Name | Structure | LCMS |
|---|---|---|
| (S)-or (R)-5- ((diethoxyphosphoryl)fluoromethyl) benzo[b]thiophene-2-carboxylic acid | 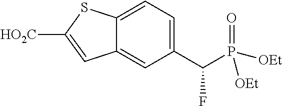 [SMILES: CCOP(=O)(OCC)[C@H](F)c1ccc2sc(C(=O)O)cc2c1] or 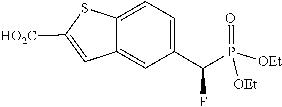 [SMILES: CCOP(=O)(OCC)[C@@H](F)c1ccc2sc(C(=O)O)cc2c1] | 347.2 [M + H]⁺ |

### Preparation of 5-(((((S)-1-isopropoxy-1-oxopropan-2-yl)amino)(phenoxy)phosphoryl)methyl)benzo[b]thiophene-2-carboxylic acid

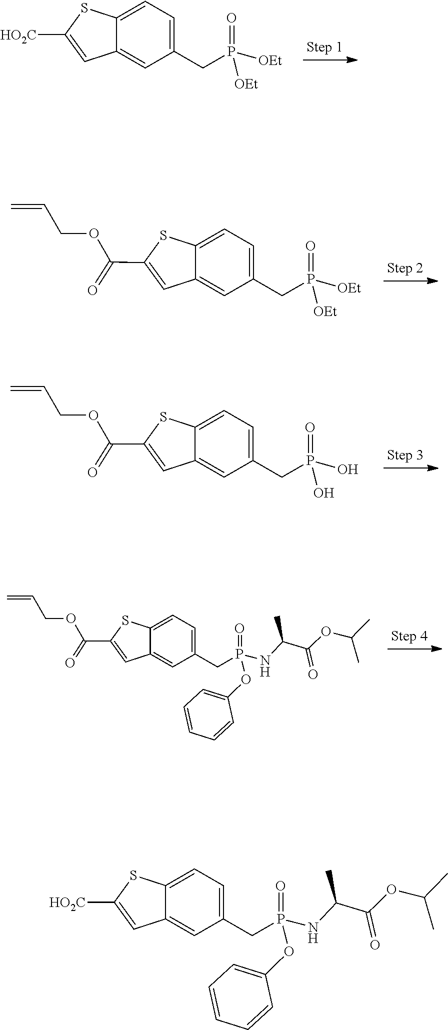 [SMILES: CCOP(=O)(Cc1ccc2sc(C(=O)O)cc2c1)OCC ; C=CCOC(=O)c1cc2cc(CP(=O)(OCC)OCC)ccc2s1 ; C=CCOC(=O)c1cc2cc(CP(=O)(O)O)ccc2s1 ; C=CCOC(=O)c1cc2cc(CP(=O)(N[C@@H](C)C(=O)OC(C)C)Oc3ccccc3)ccc2s1 ; CC(C)OC(=O)[C@H](C)NP(=O)(Cc1ccc2sc(C(=O)O)cc2c1)Oc1ccccc1]

### Step 1: Preparation of allyl 5-((diethoxyphosphoryl)methyl)benzo[b]thiophene-2-carboxylate

To a suspension of 5-((diethoxyphosphoryl)methyl)benzo[b]thiophene-2-carboxylic acid (1.0 g, 3.0 mmol, 1.0 eq) and K₂CO₃ (839 mg, 6.1 mmol, 2.0 eq) in DMF (20 mL) was added 3-bromoprop-1-ene (440 mg, 3.6 mmol, 1.2 eq). The mixture was stirred at room temperature for 14 h and poured over water (30 mL). The mixture was extracted with EtOAc (25 mL×3). The combined organic layers were dried over Na₂SO₄, filtered, and concentrated under reduced pressure, the residue was purified by column chromatography to give allyl 5-((diethoxyphosphoryl)methyl)benzo[b]thiophene-2-carboxylate (0.980 g, 2.7 mmol, 88% yield) as a light-yellow solid. LCMS (ESI) m/z=369 [M+H]⁺; tH NMR (400 MHz, CDCl₃) δ 8.04 (s, 1H), 7.84-7.77 (m, 2H), 7.45-7.37 (m, 1H), 6.18-5.94 (m, 1H), 5.49-5.39 (m, 1H), 5.36-5.28 (m, 1H), 4.87-4.83 (m, 2H), 4.08-3.97 (m, 4H), 3.27 (d, J=21.4 Hz, 2H), 1.25 (t, J=7.1 Hz, 6H).

### Step 2: Preparation of ((2-((allyloxy)carbonyl)benzo[b]thiophen-5-yl)methyl)phosphonic acid

To a solution of allyl 5-((diethoxyphosphoryl)methyl)benzo[b]thiophene-2-carboxylate (980 mg, 2.7 mmol, 1.0 eq) in CH₂Cl₂ (15 mL) was added bromotrimethylsilane (3 mL). The mixture was stirred at room temperature for 14 h and subsequently concentrated under reduced pressure. The resulting residue was triturated with H₂O (5 mL) and the resulting precipitates were filtered. The filter cake was washed with H₂O (5 mL×2) and dried under reduced pressure to give ((2-((allyloxy)carbonyl)benzo[b]thiophen-5-yl)methyl)phosphonic acid (0.710 g, 2.3 mmol, 86% yield) as a white solid. LCMS (ESI) m/z=313 [M+H]⁺; ¹H NMR (400 MHz, DMSO-d₆) δ 8.20 (s, 1H), 7.96 (d, J=8.4 Hz, 1H), 7.92-7.83 (m, 1H), 7.50-7.38 (m, 1H), 6.12-5.98 (m, 1H), 5.46-5.38 (m, 1H), 5.32-5.27 (m, 1H), 4.85-4.80 (m, 2H), 3.08 (d, J=21.2 Hz, 2H).

### Step 3: Preparation of allyl 5-(((((S)-1-isopropoxy-1-oxopropan-2-yl)amino)(phenoxy)phosphoryl)methyl)benzo[b]thiophene-2-carboxylate

To a cooled (0° C.) solution (under a constant stream of N₂ (g)) of ((2-((allyloxy)carbonyl)benzo[b]thiophen-5-yl)methyl)phosphonic acid (2.80 g, 8.96 mmol, 1 eq) and catalytic DMF (1 drop) in dry CH₂Cl₂ (50 mL) was added oxalyl chloride (3.40 g, 26.8 mmol, 3 eq). After effervescence of gas ceased, the mixture was warmed at 40° C. After 2 h, the mixture was cooled to room temperature and concentrated in vacuo to give yellow solids. The solids were subsequently diluted CH₂Cl₂ (50 mL) and cooled to 0° C. To the cooled solution was added phenol (0.843 g, 8.96 mmol, 1 eq) and Et₃N (4.53 g, 44.8 mmol, 5 eq). After complete addition, the mixture was warmed to room temperature and stirred for 1 h, followed by introduction of propan-2-yl (2S)-2-aminopropanoate (1.75 g, 13.4 mmol, 1.5 eq) to the mixture. After stirring for an additional 2 h, the mixture was concentrated to dryness. The residue was purified by C18 column (elution 50%-80% acetonitrile in water) to give allyl 5-(((((S)-1-isopropoxy-1-oxopropan-2-yl)amino)(phenoxy)phosphoryl)methyl)benzo[b]thiophene-2-carboxylate (2.23 g, 4.44 mmol, 49.6% yield) as white solids. LCMS (ESI) m/z=502.0 [M+H]⁺; ¹H NMR (400 MHz, CDCl₃) δ 8.05 (d, J=6.9 Hz, 1H), 7.91-7.80 (m, 2H), 7.53-7.43 (m, 1H), 7.29 (d, J=8.1 Hz, 2H), 7.18-7.09 (m, 3H), 6.05 (ddd, J=16.1, 10.9, 5.6 Hz, 1H), 5.48-5.40 (m, 1H), 5.32 (dd, J=10.4, 1.2 Hz, 1H), 4.98-4.87 (m, 1H), 4.85 (d, J=5.7 Hz, 2H), 4.04-3.85 (m, 1H), 3.44 (dd, J=20.7, 14.1 Hz, 2H), 3.12 (t, J=10.9 Hz, 1H), 1.21-1.10 (m, 9H).

### Step 4: Preparation of 5-(((((S)-1-isopropoxy-1-oxopropan-2-yl)amino)(phenoxy)phosphoryl)methyl)benzo[b]thiophene-2-carboxylic acid

A solution of allyl 5-(((((S)-1-isopropoxy-1-oxopropan-2-yl)amino)(phenoxy)phosphoryl)methyl)benzo[b]thiophene-2-carboxylate (90 mg, 0.1794 mmol, 1 eq), pyrrolidine (12.7 mg, 179 μmol, 1 eq), Pd(PPh₃)₄ (10.3 mg, 8.97 μmol, 0.05 eq) in CH₂Cl₂ (5 mL) was stirred under N₂ (g). After 2 h, the reaction was concentrated in vacuo. The residue was purified by C18 column (elution 30%-70% acetonitrile in water) to yield 5-(((((S)-1-isopropoxy-1-oxopropan-2-yl)amino)(phenoxy)phosphoryl)methyl)benzo[b]thiophene-2-carboxylic acid (64.0 mg, 0.1386 mmol, 77.4% yield) as white solids. LCMS (ESI) m/z 462.1 [M+H]⁺.

The intermediates in the table below were prepared using the described above for synthesis of 5-(((((S)-1-isopropoxy-1-oxopropan-2-yl)amino)(phenoxy)phosphoryl)methyl)benzo[b]thiophene-2-carboxylic acid and utilizing the appropriate utilizing the appropriate starting materials and modifications.

| Name | Structure | LCMS |
|---|---|---|
| 5-(fluoro((((S)-1-isopropoxy-1- oxopropan-2- yl)amino)(phenoxy)phosphoryl) methyl)benzo[b]thiophene-2- carboxylic acid | 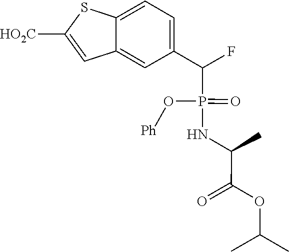 [SMILES: CC(C)OC(=O)[C@H](C)NP(=O)(Oc1ccccc1)C(F)c1ccc2sc(C(=O)O)cc2c1] | 480.1 [M + H]⁺ |
| 5-(((((S)-1-isopropoxy-1- oxopropan-2-yl)amino)(2,2,2- trifluoroethoxy)phosphoryl)methyl) benzo[b]thiophene-2-carboxylic acid | 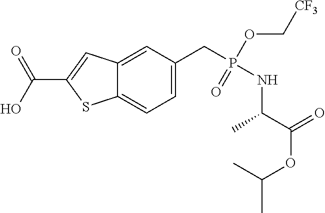 [SMILES: CC(C)OC(=O)[C@H](C)NP(=O)(Cc1ccc2sc(C(=O)O)cc2c1)OCC(F)(F)F] | 468.2 [M + H]⁺ |
| 5-(((4-chlorophenoxy)(((S)-1- isopropoxy-1-oxopropan-2- yl)amino)phosphoryl)methyl)benzo [b]thiophene-2-carboxylic acid | 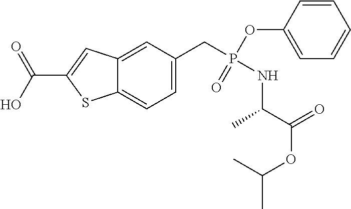 [SMILES: CC(C)OC(=O)[C@H](C)NP(=O)(Cc1ccc2sc(C(=O)O)cc2c1)Oc1ccccc1] | 496.2 [M + H]⁺ |
| 5-(((2-chlorophenoxy)(((S)-1- isopropoxy-1-oxopropan-2- yl)amino)phosphoryl)methyl)benzo [b]thiophene-2-carboxylic acid | 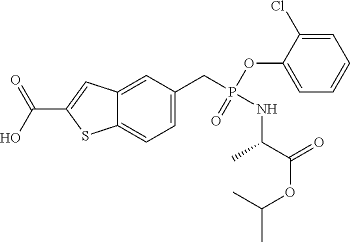 [SMILES: CC(C)OC(=O)[C@H](C)NP(=O)(Cc1ccc2sc(C(=O)O)cc2c1)Oc1ccccc1Cl] | 496.2 [M + H]⁺ |

### Synthesis of 5-(((((S)-1-(benzyloxy)-1-oxopropan-2-yl)amino)(phenoxy)phosphoryl)difluoromethyl)benzo[b]thiophene-2-carboxylic acid

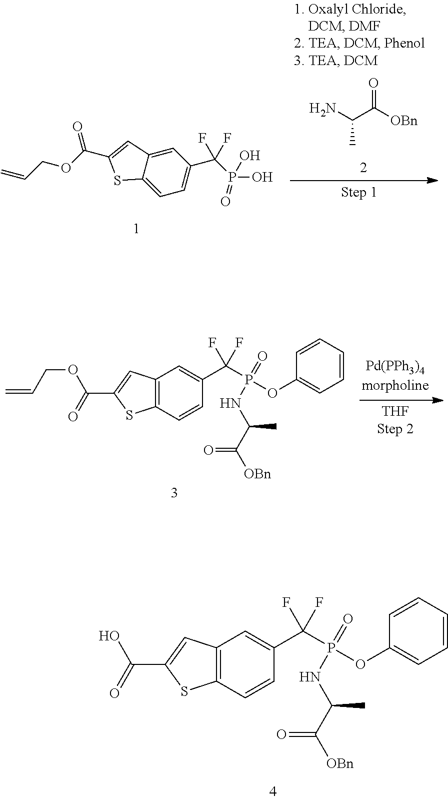 [SMILES: C=CCOC(=O)c1cc2cc(C(F)(F)P(=O)(O)O)ccc2s1 ; C[C@H](N)C(=O)OCc1ccccc1 ; C=CCOC(=O)c1cc2cc(C(F)(F)P(=O)(N[C@@H](C)C(=O)OCc3ccccc3)Oc3ccccc3)ccc2s1 ; C[C@H](NP(=O)(Oc1ccccc1)C(F)(F)c1ccc2sc(C(=O)O)cc2c1)C(=O)OCc1ccccc1]

### Step 1: Preparation of allyl 5-(((((S)-1-(benzyloxy)-1-oxopropan-2-yl)amino)(phenoxy)phosphoryl)difluoromethyl)benzo[b]thiophene-2-carboxylate

To a solution of ((2-((allyloxy)carbonyl)benzo[b]thiophen-5-yl)difluoromethyl)phosphonic acid (300 mg, 861 μmol, 1 eq) in DCM (5 mL) at 0° C. were added 2 drops of DMF (cat.) followed by dropwise addition of oxalyl chloride (220 μL, 2.58 mmol, 3 eq). The reaction was warmed up to room temperature and stirred for 16 h. The reaction mixture was concentrated under reduced pressure and dried completely under high vacuum for 30 min. to give a yellow solid. The yellow solid was diluted in DCM (5 mL) and cooled to 0° C. A solution of phenol (89.1 mg, 947 μmol, 1.1 eq) and triethylamine (599 μL, 4.30 mmol, 5 eq) in DCM (1 mL, dried on Na₂SO₄) was slowly added to the yellow solution. The reaction mixture was stirred at 0° C. for 2 min. and then warmed up to room temperature and stirred for 2 h. A solution of L-alanine benzyl ester p-toluenesulfonate salt (453 mg, 1.29 mmol, 1.5 eq) in DCM (1 mL, dried on Na₂SO₄) was slowly added to the yellow solution. The reaction mixture was stirred at room temperature for 18 h. The solvent was removed in vacuo. The crude product was purified by reverse phase chromatography on a 50 g Cis cartridge eluting with a gradient of 5-100% acetonitrile in water. The pure fractions were combined and concentrated under reduced pressure to give allyl 5-(((((S)-1-(benzyloxy)-1-oxopropan-2-yl)amino)(phenoxy)phosphoryl)difluoromethyl)benzo[b]thiophene-2-carboxylate (196 mg, 38.8%) as a yellow oil. LCMS (ESI) m/z=586.2.

The intermediates in the table below were prepared using the method described above in Step 1 for the preparation of allyl 5-(((((S)-1-(benzyloxy)-1-oxopropan-2-yl)amino)(phenoxy)phosphoryl)difluoromethyl)benzo[b]thiophene-2-carboxylate and utilizing the appropriate starting materials and modifications.

| Name | Structure | NMR; LCMS |
|---|---|---|
| butyl (2S)-1-(((2- ((allyloxy)carbonyl) benzo[b]thiophen-5- yl)difluoromethyl) (phenoxy)phosphoryl)-4,4- difluoropyrrolidine-2- carboxylate | 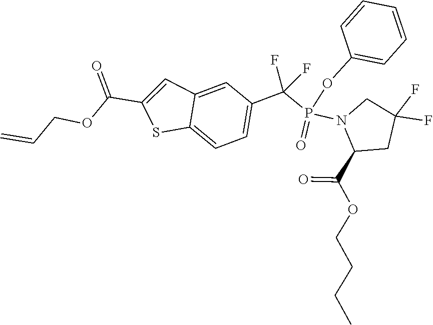 [SMILES: C=CCOC(=O)c1cc2cc(C(F)(F)P(=O)(Oc3ccccc3)N3CC(F)(F)C[C@H]3C(=O)OCCCC)ccc2s1] | ¹H NMR (400 MHz, CDCl₃) δ 8.19 (s, 1H), 8.12 (s, 1H), 7.99- 7.91 (m, 1H), 7.79-7.73 (m, 1H), 7.35-7.02 (m, 5H), 6.12-5.98 (m, 1H), 5.50-5.41 (m, 1H), 5.38- 5.28 (m, 1H), 4.89-4.84 (m, 2H), 4.81-4.73 (m, 0.8H), 4.47- 4.38 (m, 0.2H), 4.29-3.96 (m, 2H), 3.88-3.64 (m, 2H), 2.76- 2.43 (m, 2H), 1.61-1.50 (m, 2H), 1.39-1.23 (m, 2H), 0.90 (t, J = 7.3 Hz, 3H); 614.2 [M + H]⁺ |
| butyl (((2- ((allyloxy)carbonyl) benzo[b]thiophen-5- yl)difluoromethyl) (phenoxy)phosphoryl)-L- prolinate | 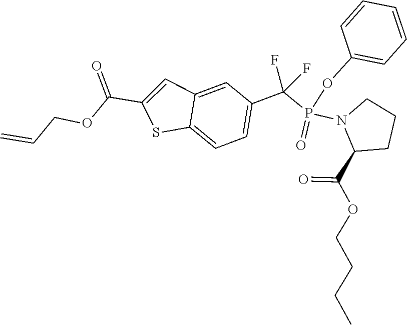 | 542.3 [M + H]⁺ |

### Step 2: Preparation of 5-(((((S)-1-(benzyloxy)-1-oxopropan-2-yl)amino)(phenoxy)phosphoryl)difluoromethyl)benzo[b]thiophene-2-carboxylic acid

To a stirred solution of allyl 5-(((((S)-1-(benzyloxy)-1-oxopropan-2-yl)amino)(phenoxy)phosphoryl)difluoromethyl)benzo[b]thiophene-2-carboxylate (196 mg, 334 μmol, 1 eq) in THF (5 mL) were added morpholine (143 μL, 1.67 mmol, 5 eq) and Pd(PPh₃)₄ (38.5 mg, 33.4 μmol, 0.10 eq) under nitrogen. The reaction mixture was stirred 1 h at room temperature. The reaction mixture was directly purified by reverse phase chromatography on a 50 g C18 cartridge eluting with a gradient of 5-100% acetonitrile in water (with 0.1% formic acid). The pure fractions were concentrated under reduced pressure to give 5-(((((S)-1-(benzyloxy)-1-oxopropan-2-yl)amino)(phenoxy)phosphoryl)difluoromethyl)benzo[b]thiophene-2-carboxylic acid (164 mg, 90.1%) as a light brown solid. ¹H NMR (400 MHz, CDCl₃) δ 8.22-8.09 (m, 1H), 7.88-7.76 (m, 2H), 7.66-7.59 (m, 1H), 7.43-7.29 (m, 7.6H), 7.26-7.15 (m, 2.4H), 5.24 (s, 1H), 5.13 (s, 1H), 4.83-4.67 (m, 0.5H), 4.62-4.44 (m, 0.5H), 4.39-4.14 (m, 1H), 1.50-1.35 (m, 3H).

The intermediates in the table below were prepared using the method described above in Step 2 for the preparation of 5-(((((S)-1-(benzyloxy)-1-oxopropan-2-yl)amino)(phenoxy)phosphoryl)difluoromethyl)benzo[b]thiophene-2-carboxylic acid and utilizing the appropriate starting materials and modifications.

| Name | Structure | LCMS |
|---|---|---|
| 5-((((S)-2-(butoxycarbonyl)-4,4- difluoropyrrolidin-1- yl)(phenoxy)phosphoryl)difluoromethyl) benzo[b]thiophene-2-carboxylic acid | 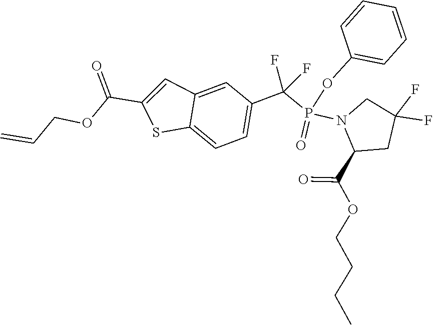 | 574.2 [M + H]⁺ |
| 5-((((S)-2- (butoxycarbonyl)pyrrolidin-1- yl)(phenoxy)phosphoryl)difluoromethyl) benzo[b]thiophene-2-carboxylic acid | 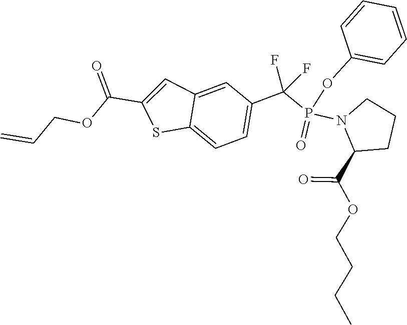 | 537.8 [M + H]⁺ |

### Synthesis of 5-(((((S)-1-butoxy-1-oxopropan-2-yl)amino)(naphthalen-1-yloxy)phosphoryl)difluoromethyl)benzo[b]thiophene-2-carboxylic acid

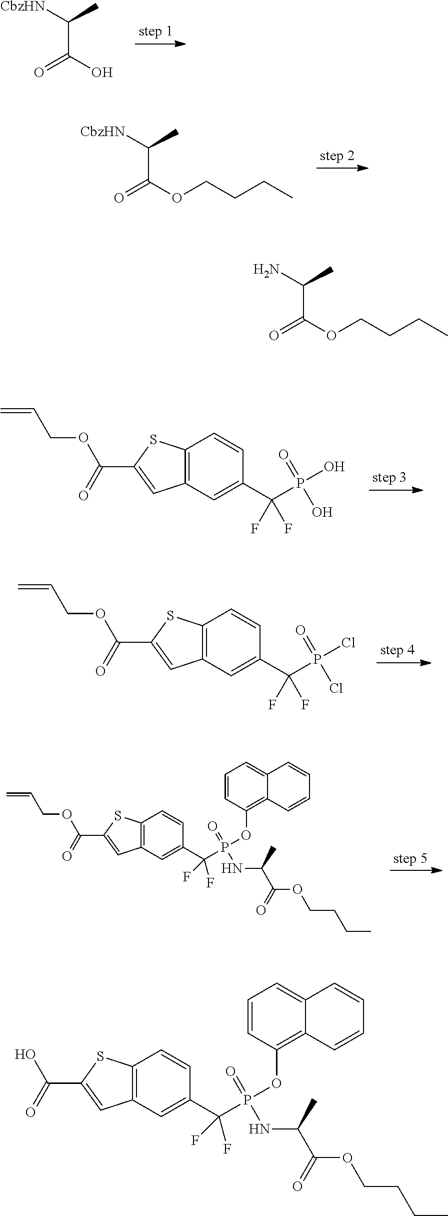

### Step 1: Preparation of butyl ((benzyloxy)carbonyl)-L-alaninate

To a solution of ((benzyloxy)carbonyl)-L-alanine (5 g, 22 mmol, 1 eq) in DMF (50 mL) was added 1-chlorobutane (2.1 g, 22 mmol, 1 eq) and dipotassium carbonate (6.2 g, 45 mmol, 2 eq), the mixture was stirred at 60° C. for 1 h to give yellow solution. The mixture was diluted with water (200 mL) and extracted with EtOAc (200 mL×2), the combined organic layers were washed with saturated brine (200 mL×2), dried over Na₂SO₄, filtered and concentrated under reduced pressure to give a residue. The residue was purified by column chromatography to give butyl ((benzyloxy)carbonyl)-L-alaninate (3.6 g, 13 mmol, 57.8% yield) as a yellow oil. LCMS (ESI) m/z=280.0

### Step 2: Preparation of butyl L-alaninate

To a solution of butyl ((benzyloxy)carbonyl)-L-alaninate (3 g, 11 mmol, 1 eq) in THF (30 mL) was added Pd/C (3 g, 10%) under N₂, the mixture was stirred at 25° C. for 2 h under H₂ (15 Psi). The reaction mixture was filtered and the filter was concentrated to give butyl L-alaninate (1.7 g, crude) as a yellow oil. LCMS (ESI) m/z=146.1 [M+H]⁺.

### Step 3: Preparation of allyl 5-((dichlorophosphoryl)difluoromethyl)benzo[b]thiophene-2-carboxylate

To a solution of ((2-((allyloxy)carbonyl)benzo[b]thiophen-5-yl)difluoromethyl)phosphonic acid (0.2 g, 0.57 mmol, 1 eq) in DCM (5 mL) was added dimethylformamide (0.42 mg, 5.7 μmol, 0.01 eq), the mixture was stirred at 0° C. for 5 min, then a solution of oxalyl chloride (0.22 g, 1.7 mmol, 3 eq) in DCM (5 mL) was added to the mixture, the mixture was stirred at 40° C. for 15 min to give yellow solution. The reaction mixture was concentrated under reduced pressure to give allyl 5-((dichlorophosphoryl)difluoromethyl)benzo[b]thiophene-2-carboxylate (0.2 g, crude) as a yellow oil. LCMS (ESI) m/z=377.0 [M+H]⁺.

### Step 4: Preparation of allyl 5-(((((S)-1-butoxy-1-oxopropan-2-yl)amino)(naphthalen-1-yloxy)phosphoryl)difluoromethyl)benzo[b]thiophene-2-carboxylate

To a solution of allyl 5-((dichlorophosphoryl)difluoromethyl)benzo[b]thiophene-2-carboxylate (0.2 g, 0.52 mmol, 1 eq) in DCM (10 mL) was added naphthalen-1-ol (60 mg, 0.42 mmol, 0.8 eq), the mixture was stirred at 0° C. for 5 min, then a solution of N,N-diisopropylethylamine (0.2 g, 1.6 mmol, 3.0 eq) in DCM (10 mL) was added to the mixture, then a solution of butyl L-alaninate (75 mg, 0.52 mmol, 1 eq) in DCM (10 mL) was added to the mixture, the mixture was stirred at 0° C. for 30 min to give yellow solution. The reaction mixture was concentrated under reduced pressure to give a residue. The residue was purified by column chromatography to give allyl 5-(((((S)-1-butoxy-1-oxopropan-2-yl)amino)(naphthalen-1-yloxy)phosphoryl)difluoromethyl)benzo[b]thiophene-2-carboxylate (0.15 g, 0.24 mmol, 46.4% yield) as a yellow oil. LCMS (ESI) m/z=602.1 [M+H]⁺.

### Step 5: Preparation of 5-(((((S)-1-butoxy-1-oxopropan-2-yl)amino)(naphthalen-1-yloxy)phosphoryl)difluoromethyl)benzo[b]thiophene-2-carboxylic acid

To a solution of allyl 5-(((((S)-1-butoxy-1-oxopropan-2-yl)amino)(naphthalen-1-yloxy)phosphoryl)difluoromethyl)benzo[b]thiophene-2-carboxylate (0.14 g, 0.23 mmol, 1 eq) in DCM (1.0 mL) was added Pd(PPh₃)₄ (27 mg, 23 μmol, 0.1 eq), the mixture was stirred at 0° C. for 5 min, then pyrrolidine (17 mg, 0.23 mmol, 1 eq) was added to the mixture, the mixture was stirred at 25° C. for 5 min to give yellow solution. The reaction mixture was concentrated under reduced pressure to give a residue. The residue was purified by reversed phase (TFA) to give 5-(((((S)-1-butoxy-1-oxopropan-2-yl)amino)(naphthalen-1-yloxy)phosphoryl)difluoromethyl)benzo[b]thiophene-2-carboxylic acid (97 mg, 0.17 mmol, 74.6% yield) as a yellow oil. LCMS (ESI) m/z=562.1 [M+H]⁺.

### Synthesis of ((2-((allyloxy)carbonyl)benzo[b]thiophen-5-yl)methyl)phosphonic acid and ((2-((allyloxy)carbonyl)benzo[b]thiophen-5-yl)fluoromethyl)phosphonic acid

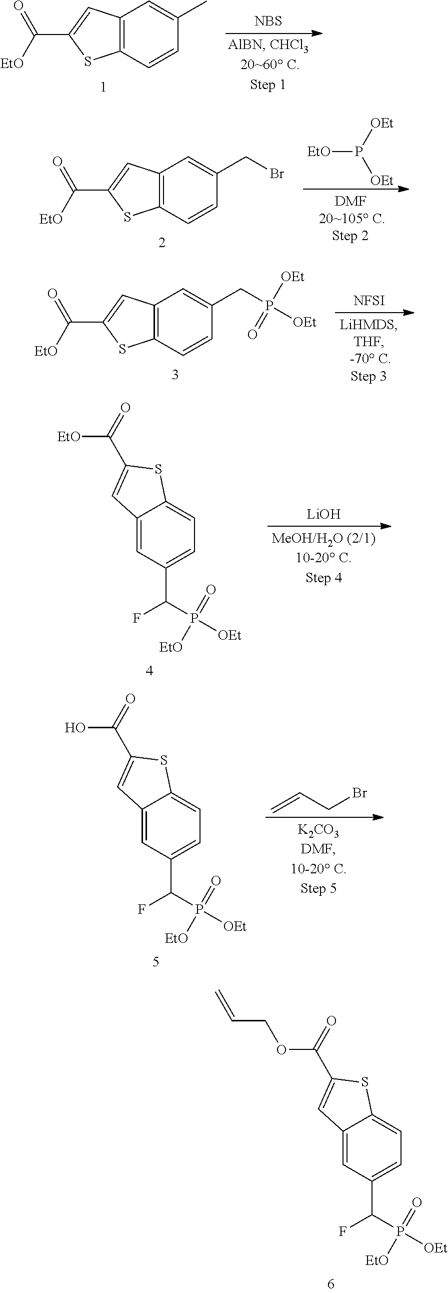

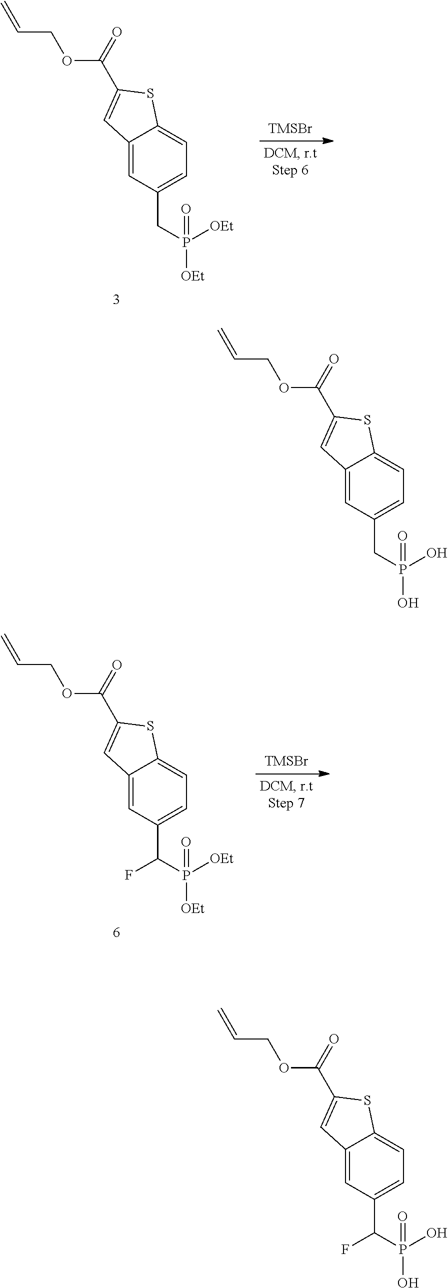 [SMILES: "TMSBr" ; C=CCOC(=O)c1cc2cc(CP(=O)(OCC)OCC)ccc2s1 ; "TMSBr" ; C=CCOC(=O)c1cc2cc(CP(=O)(O)O)ccc2s1 ; C=CCOC(=O)c1cc2cc(C(F)P(=O)(OCC)OCC)ccc2s1 ; C=CCOC(=O)c1cc2cc(C(F)P(=O)(O)O)ccc2s1]

Ethyl 5-methylbenzo[b]thiophene-2-carboxylate was prepared according to the procedure described in WO 2016/100184 (e.g., paragraphs [0281]-[0284], which are incorporated by reference herein).

### Step 1: Preparation of ethyl 5-(bromomethyl)benzo[b]thiophene-2-carboxylate

To a solution of ethyl 5-methylbenzo[b]thiophene-2-carboxylate (1, 7.3 kg, 33.1 mol, 1.0 eq.) in CHCl₃ (58 L) stirred at 20° C. was added AIBN (544 g, 3.31 mol, 0.10 eq.) and NBS (6.19 kg, 34.8 mol, 1.05 eq.). The mixture was heated from 30° C. to 50° C. over 4 h and was then heated to 60° C. and stirred for 12 hours. After completion, the reaction mixture was cooled to 10° C. and 15% Na₂SO₃ (20 L) was added. The organic layers were washed with H₂O (20 L*2), dried over Na₂SO₄, and concentrated under reduced pressure at 45° C. to afford ethyl 5-(bromomethyl)benzo[b]thiophene-2-carboxylate (2, 9.50 kg, 23.5 mol, 70.9% yield, 74.0% purity) as a yellow solid. LCMS (ESI): m/z=298.9 [M+H]⁺. ¹H NMR (400 MHz, CDCl₃) δ 8.034 (s, 1H), 7.89-7.84 (m, 2H), 7.50 (d, J=9.6 Hz, 1H), 4.64 (s, 2H), 4.45-4.40 (m, 2H), 1.45-1.41 (m, 3H).

### Step 2: Preparation of ethyl 5-((diethoxyphosphoryl)methyl)benzo[b]thiophene-2-carboxylate

To a solution of ethyl 5-(bromomethyl)benzo[b]thiophene-2-carboxylate (2, 9.50 kg, 31.8 mol, 1.0 eq.) in DMF (28.5 L) stirred at 20° C. was added triethyl phosphite (5.8 kg, 34.9 mol, 1.1 equiv). The mixture was heated to 100° C. and stirred for 5 h. After completion, the reaction mixture was cooled to 15° C., poured into H₂O (50.0 L), and extracted with EtOAc (20 L*2). The combined organics were washed with H₂O (20 L*2) and brine (10 L), dried over Na₂SO₄, and concentrated under reduced pressure at 45° C. to give a residue. Crude residue was purified by silica gel chromatography (Petroleum ether/Ethyl acetate=100/1 to 1/1, Petroleum ether/Ethyl acetate=0/1) to afford ethyl 5-((diethoxyphosphoryl)methyl)benzo[b]thiophene-2-carboxylate (3, 5.24 kg, 14 mol, 44.4% yield) as yellow solid. LCMS (ESI): m/z=356.9 [M+H]⁺. ¹H NMR (400 MHz, CDCl₃) δ 8.02 (s, 1H), 7.82-7.80 (m, 2H), 7.41 (d, J=2.0 Hz, 1H), 4.44-4.41 (m, 2H), 4.05-4.01 (m, 4H), 3.27 (d, J=21.6 Hz, 2H), 1.46-1.42 (m, 3H), 1.27-1.23 (m, 6H).

### Step 3: Preparation of ethyl 5-((diethoxyphosphoryl)fluoromethyl)benzo[b]thiophene-2-carboxylate

Three batches were carried out in parallel.

To a solution of ethyl 5-((diethoxyphosphoryl)methyl)benzo[b]thiophene-2-carboxylate (3, 250 g, 702 mmol, 1.00 eq) and N-(benzenesulfonyl)-N-fluorobenzenesulfonamide (221 g, 702 mmol, 1.00 eq) in THF (2.50 L) was added dropwise LiHMDS (1 M, 702 mL, 1.00 eq) at −70° C. under N₂. The mixture was stirred at −70° C. for 3 h. Following completion, the reaction mixture was poured into saturated NH₄Cl aqueous solution (5.00 L) slowly at 0° C. and the mixture was stirred at 0° C. for 0.5 hr. Then three batches were combined to workup. The mixture was extracted with ethyl acetate (5.00 L*3). The organic layers were combined, washed with brine (5.00 L), dried over anhydrous Na₂SO₄ and concentrated under reduced pressure. The residue was purified by column chromatography (SiO₂, petroleum ether/ethyl acetate=1/0-3/1, R_{f}=0.30, petroleum ether/ethyl acetate=1/1) to give ethyl 5-((diethoxyphosphoryl)fluoromethyl)benzo[b]thiophene-2-carboxylate (4, 357 g, 928 mmol, 44.1% yield) as yellow oil. LCMS (ESI): m/z=375.0 [M+H]⁺. ¹H NMR (400 MHz, CDCl₃) δ 8.07 (s, 1H), 8.00 (s, 1H), 7.90 (d, J=8.4 Hz, 1H), 7.59 (d, J=8.8 Hz, 1H), 5.88-5.75 (m, 1H), 4.45-4.40 (m, 2H), 4.15-4.05 (m, 4H), 1.45-1.41 (m 3H), 1.31-1.28 (m, 6H).

### Step 4: Preparation of 5-((diethoxyphosphoryl)fluoromethyl)benzo[b]thiophene-2-carboxylic acid

To a solution of ethyl 5-((diethoxyphosphoryl)fluoromethyl)benzo[b]thiophene-2-carboxylate (4, 252 g, 673 mmol, 1.00 eq) in MeOH (1.80 L) was added H₂O (760 mL) and LiOH·H₂O (56.5 g, 1.35 mol, 2.00 eq) at 10-20° C. under N₂. The mixture was stirred at 10-20° C. for 1 hr. TLC (petroleum ether/ethyl acetate=1/1) showed that compound 4 was consumed (R_{f}=0.30) and desired spot (R_{f}=0.10) was formed. The reaction mixture was quenched by H₂O (2.50 L) and then adjusted pH to 3-4 with HCl (aq. 1M). The mixture was extracted with dichloromethane (2.50 L*3). The combined organic layers were dried over anhydrous Na₂SO₄ and concentrated under reduced pressure to give 5-((diethoxyphosphoryl)fluoromethyl)benzo[b]thiophene-2-carboxylic acid (5, 222 g, 626 mmol, 93.0% yield) as a white solid.

### Step 5: Preparation of allyl 5-((diethoxyphosphoryl)fluoromethyl)benzo[b]thiophene-2-carboxylate

Two batches were carried out in parallel.

To a solution of 5-((diethoxyphosphoryl)fluoromethyl)benzo[b]thiophene-2-carboxylic acid (5, 178 g, 514 mmol, 1.00 eq) in DMF (1.78 L) was added K₂CO₃ (142 g, 1.03 mol, 2.00 eq) and allyl bromide (68.4 g, 565 mmol, 1.10 eq) at 10-20° C. The mixture was stirred at 10-20° C. for 12 h. TLC (petroleum ether/ethyl acetate=0/1) showed that compound 5 was consumed (R_{f}=0.60) and a new spot (R_{f}=0.70) was formed. The reaction mixture was diluted with H₂O (6.00 L), extracted with ethyl acetate (2.00 L*3). The organic layers were combined. The mixture was washed with brine (2.00 L) and NH₄Cl (2.00 L), dried over anhydrous Na₂SO₄ and concentrated under reduced pressure to give allyl 5-((diethoxyphosphoryl)fluoromethyl)benzo[b]thiophene-2-carboxylate (6, 388 g, 985 mmol, 95.8% yield) as yellow oil.

### Step 6: Preparation of ((2-((allyloxy)carbonyl)benzo[b]thiophen-5-yl)methyl)phosphonic acid

To a solution of allyl 5-((diethoxyphosphoryl)methyl)benzo[b]thiophene-2-carboxylate (3, 50 g, 136 mmol, 1.0 eq.) in DCM (5 L) was added TMSBr (411 g, 2.71 mol, 20.0 eq.) dropwise at 0° C. After addition, the reaction mixture was allowed to warm to room temperature and stirred for an additional 12 h. The reaction progress was monitored by LCMS. After completion, the reaction mixture was concentrated under reduced pressure and water was added. The resulting mixture was filtered and the filter cake was washed with water (2 L), then dried in vacuum to afford ((2-((allyloxy)carbonyl)benzo[b]thiophen-5-yl)methyl)phosphonic acid (40.3 g, 129 mmol, 95%) as a yellow solid. ¹H NMR (400 MHz, DMSO-d₆) δ 8.20 (s, 1H), 7.96 (d, J=8.4 Hz, 1H), 7.87 (s, 1H), 7.43 (d, J=8.4 Hz, 1H), 6.10-5.98 (m, 1H), 5.46-5.37 (m, 1H), 5.33-5.24 (m, 1H), 4.86-4.77 (m, 2H), 3.08 (d, J=21.2 Hz, 2H). LCMS (ESI) m/z=313.1 [M+H]⁺.

### Step 7: Preparation of ((2-((allyloxy)carbonyl)benzo[b]thiophen-5-yl)fluoromethyl)phosphonic acid

To a solution of allyl 5-((diethoxyphosphoryl)fluoromethyl)benzo[b]thiophene-2-carboxylate (6, 5.2 g, 13.5 mmol, 1.0 eq.) in DCM (500 mL) was added TMSBr (41.1 g, 270 mmol, 20.0 eq.) dropwise at 0° C. After the addition, the reaction mixture was allowed to warm to room temperature and stirred for an additional 12 h. The reaction progress was monitored by LCMS. After completion, the reaction mixture was concentrated under reduced pressure and water was added. The resulting mixture was filtered and the filter cake was washed with water (200 mL), then dried in vacuum to afford ((2-((allyloxy)carbonyl)benzo[b]thiophen-5-yl)fluoromethyl)phosphonic acid (3.1 g, 9.37 mmol, 69%) as a yellow solid. ¹H NMR (400 MHz, DMSO-d₆) δ 8.32 (s, 1H), 8.14-8.05 (m, 2H), 7.59 (d, J=8.4 Hz, 1H), 6.12-5.99 (m, 1H), 5.84 (dd, J=44.3, 8.2 Hz, 1H), 5.49-5.40 (m, 1H), 5.35-5.27 (m, 1H), 4.88-4.81 (m, 2H). LCMS (ESI): m/z=329.1 [M−H]⁻.

### Chiral Separation of allyl 5-((diethoxyphosphoryl)fluoromethyl)benzo[b]thiophene-2-carboxylate

Rac-allyl 5-((diethoxyphosphoryl)fluoromethyl)benzo[b]thiophene-2-carboxylate (617 g, 1.62 mol, 1.00 eq) was purified by SFC to give allyl (S)-5-((diethoxyphosphoryl)fluoromethyl)benzo[b]thiophene-2-carboxylate (Peak 1) and allyl (R)-5-((diethoxyphosphoryl)fluoromethyl)benzo[b]thiophene-2-carboxylate (Peak 2).

Preparative SFC method: Instrument: Waters 350 Preparative SFC. Column: REGIS (S,S) WHELK-O1, 250×50 mm I.D., 10 μm. Mobile phase: A for CO₂ and B for MEOH (Neu). Gradient: B 30%. Flow rate: 220 g/min. Back pressure: 100 bar. Column temperature: 35° C. Wavelength: 220 nm. Cycle-time: 3.3 min.

Analytical SFC method: Column: Kromasil (S,S) WHELK-01, 50×4.6 mm I.D., 3.5 μm. Mobile phase: A for CO₂ and B for MEOH (0.05% DEA). Gradient: B 5 to 40% Flow rate: 3 mL/min. Back pressure: 100 bar. Column temperature: 35° C. Wavelength: 220 nm.

allyl (S)-5-((diethoxyphosphoryl)fluoromethyl)benzo[b]thiophene-2-carboxylate (Peak 1, 267 g, 685 mmol, 39.7% yield, >99% ee, RT=1.36 min) was obtained as yellow oil. LCMS (ESI): m/z=387.1 [M+H]⁺.

allyl (R)-5-((diethoxyphosphoryl)fluoromethyl)benzo[b]thiophene-2-carboxylate (Peak 2, 270 g, 676 mmol, 39.2% yield, >99% ee, RT=1.55 min) was obtained as yellow oil. LCMS (ESI): m/z=387.1 [M+H]⁺.

Assignment of absolute stereochemical configuration was made by comparison of experimental vibrational circular dichroism (VCD) spectra with theoretical VCD spectra obtained from DFT calculations.

The intermediates in the table below were prepared using the method described above in Step 7 for the preparation of ((2-((allyloxy)carbonyl)benzo[b]thiophen-5-yl)fluoromethyl)phosphonic acid and utilizing the appropriate starting materials and modifications.

| Name | Structure | LCMS |
|---|---|---|
| (S)-((2- ((allyloxy)carbonyl)benzo[b]thiophen-5- yl)fluoromethyl)phosphonic acid | 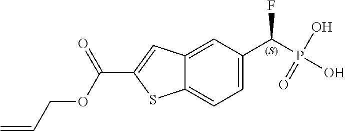 | 329 [M − H]⁻ |
| (R)-((2- ((allyloxy)carbonyl)benzo[b]thiophen-5- yl)fluoromethyl)phosphonic acid | 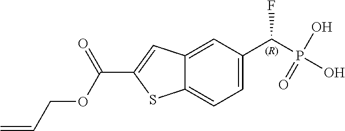 | 329 [M − H]⁻ |

### Synthesis of 5-(((2,6-dimethylphenoxy)(((S)-1-oxo-1-propoxypropan-2-yl)amino)phosphoryl)methyl)benzo[b]thiophene-2-carboxylic acid

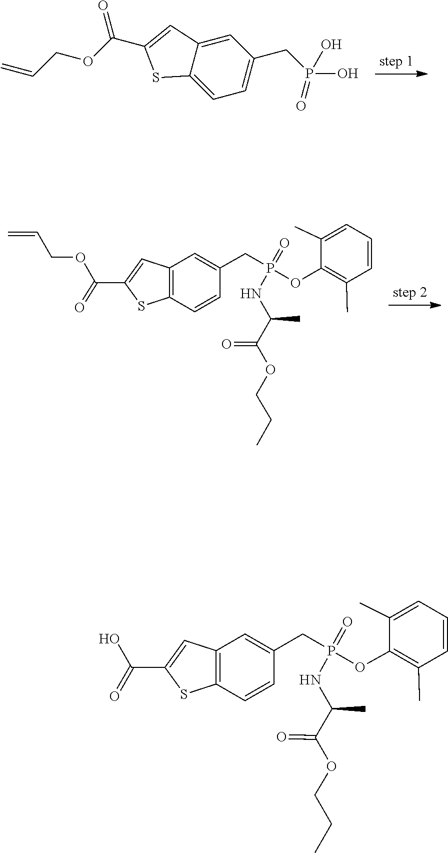 [SMILES: C=CCOC(=O)c1cc2cc(CP(=O)(O)O)ccc2s1 ; C=CCOC(=O)c1cc2cc(CP(=O)(N[C@@H](C)C(=O)OCCC)Oc3c(C)cccc3C)ccc2s1 ; CCCOC(=O)[C@H](C)NP(=O)(Cc1ccc2sc(C(=O)O)cc2c1)Oc1c(C)cccc1C]

### Step 1: Preparation of allyl 5-(((2,6-dimethylphenoxy)(((S)-1-oxo-1-propoxypropan-2-yl)amino)phosphoryl)methyl)benzo[b]thiophene-2-carboxylate

To a suspension of ((2-((allyloxy)carbonyl)benzo[b]thiophen-5-yl)methyl)phosphonic acid (0.150 g, 480 μmol, 1 eq) and 1 drop of DMF (cat.) in DCM (4 mL) at 0° C. was added oxalyl chloride (122 μL, 1.44 mmol, 3 eq) and the mixture was stirred for 2 days at room temperature. The reaction was concentrated under reduced pressure and then dried under high vacuum for 20 min. The crude residue was diluted in toluene (5 mL). A solution of 2,6-dimethylphenol (58.6 mg, 480 μmol, 1 eq) and triethylamine (333 μL, 2.40 mmol, 5 eq) in toluene (2 mL, dried with sodium sulfate) was added to the mixture and stirred at 90° C. for 3 h. Propyl (2S)-2-aminopropanoate hydrochloride (80.4 mg, 480 μmol, 1 eq) was added at once at 90° C. and the reaction was stirred at 90° C. for 2 h. The reaction was cooled down to room temperature and quenched with 2 drops of water and toluene was removed under reduced pressure. The product was purified by reverse phase chromatography on a 100 g Cis cartridge eluting with a gradient of 5-100% acetonitrile in water (with 0.1% formic acid) to afford allyl 5-(((2,6-dimethylphenoxy)(((S)-1-oxo-1-propoxypropan-2-yl)amino)phosphoryl)methyl)benzo[b]thiophene-2-carboxylate (50.0 mg, 19.6%) as a pale yellow oil. LCMS (ESI) m/z=530.4 [M+H]⁺.

The intermediates in the table below were prepared using the method described above in Step 1 for the preparation of allyl 5-(((2,6-dimethylphenoxy)(((S)-1-oxo-1-propoxypropan-2-yl)amino)phosphoryl)methyl)benzo[b]thiophene-2-carboxylate and utilizing the appropriate starting materials and modifications.

| Name | Structure | NMR; LCMS |
|---|---|---|
| allyl 5-((((2-methyl-3- oxo-3- propoxypropyl)amino) (phenoxy)phosphoryl) methyl)benzo[b]thiophene- 2-carboxylate | 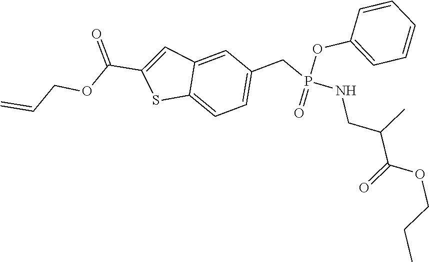 [SMILES: C=CCOC(=O)c1cc2cc(CP(=O)(NCC(C)C(=O)OCCC)Oc3ccccc3)ccc2s1] | ¹H NMR (400 MHz, CDCl₃) δ 8.07-8.02 (m, 1H), 7.88-7.80 (m, 2H), 7.50-7.43 (m, 1H), 7.33-7.27 (m, 2H), 7.19-7.10 (m, 3H), 6.11-5.99 (m, 1H), 5.49-5.41 (m, 1H), 5.36-5.30 (m, 1H), 4.89-4.83 (m, 2H), 4.03-3.85 (m, 2H), 3.47-3.36 (m, 2H), 3.14-2.95 (m, 3H), 2.43-2.31 (m, 1H), 1.64-1.51 (m, 2H), 1.01 (d, J = 7.3 Hz, 3H), 0.92-0.83 (m, 3H); 516.4 [M + H]⁺ |
| butyl (2S)-1-(((2- ((allyloxy)carbonyl)ben- zo[b]thiophen-5- yl)methyl)(phenoxy)phos- phoryl)-4,4- difluoropyrrolidine-2- carboxylate | 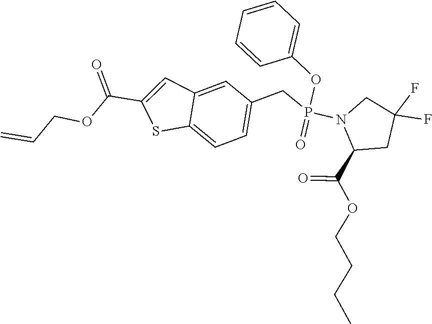 [SMILES: C=CCOC(=O)c1cc2cc(CP(=O)(Oc3ccccc3)N3CC(F)(F)C[C@H]3C(=O)OCCCC)ccc2s1] | ¹H NMR (400 MHz, CDCl₃) δ 8.10-8.02 (m, 1H), 8.00-7.89 (m, 1H), 7.88-7.80 (m, 1H), 7.63-7.50 (m, 1H), 7.37-7.10 (m, 5H), 6.14-5.98 (m, 1H), 5.49-5.42 (m, 1H), 5.39-5.28 (m, 1H), 4.89-4.83 (m, 2H), 4.73-4.67 (m, 0.4H), 4.30- 4.23 (m, 0.6H), 4.23-4.02 (m, 2H), 3.82-3.59 (m, 1H), 3.58- 3.41 (m, 2H), 3.31-3.04 (m, 1H), 2.54-2.20 (m, 2H), 1.70- 1.48 (m, 2H), 1.45-1.15 (m, 2H), 0.98-0.88 (m, 3H); 578.4 [M + H]⁺ |
| ally1 5-(((((S)-1-oxo-1- propoxybutan-2- yl)amino)(phenoxy)phos- phoryl)methyl)benzo [b]thiophene-2- carboxylate | 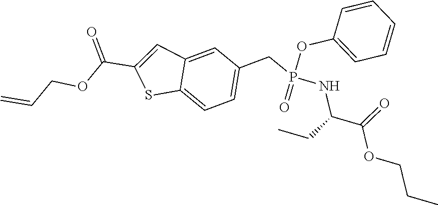 [SMILES: C=CCOC(=O)c1cc2cc(CP(=O)(N[C@@H](CC)C(=O)OCCC)Oc3ccccc3)ccc2s1] | ¹H NMR (400 MHz, CDCl₃) δ 8.09-8.00 (m, 1H), 7.93-7.81 (m, 2H), 7.56-7.44 (m, 1H), 7.33-7.27 (m, 2H), 7.19-7.04 (m, 3H), 6.12-6.00 (m, 1H), 5.49-5.42 (m, 1H), 5.37-5.30 (m, 1H), 4.89-4.82 (m, 2H), 4.05-3.87 (m, 3H), 3.51-3.39 (m, 2H), 3.33-3.07 (m, 1H), 1.63-1.46 (m, 4H), 0.93-0.83 (m, 3H), 0.78-0.59 (m, 3H); 516.2 [M + H]⁺ |
| allyl 5-((((4-oxo-4- propoxybutan-2- yl)amino)(phenoxy)phos- phoryl)methyl)benzo [b]thiophene-2- carboxylate | 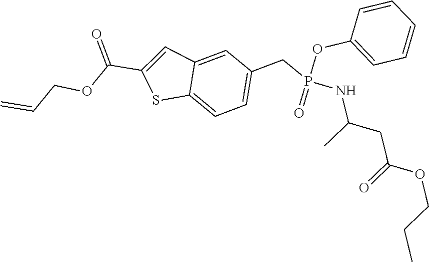 [SMILES: C=CCOC(=O)c1cc2cc(CP(=O)(NC(C)CC(=O)OCCC)Oc3ccccc3)ccc2s1] | ¹H NMR (400 MHz, CDCl₃) δ 8.07-8.02 (m, 1H), 7.90-7.78 (m, 2H), 7.50-7.44 (m, 1H), 7.34-7.27 (m, 2H), 7.21-7.08 (m, 3H), 6.13-5.99 (m, 1H), 5.49-5.41 (m, 1H), 5.36-5.30 (m, 1H), 4.89 (m, 2H) 4.03- 3.86 (m, 2H), 3.80-3.64 (m, 1H), 3.48-3.36 (m, 2H), 3.33- 3.12 (m, 1H), 2.34-2.14 (m, 2H), 1.67-1.50 (m, 2H), 1.10- 0.97 (m, 3H), 0.95-0.85 (m, 3H); 516.2 [M + H]⁺ |
| butyl (((2- ((allyloxy)carbonyl)ben- zo[b]thiophen-5- yl)methyl)(phenoxy)phos- phoryl)-L-prolinate | 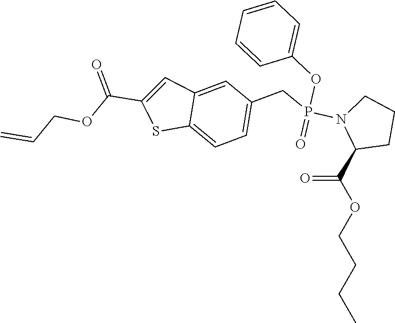 [SMILES: C=CCOC(=O)c1cc2cc(CP(=O)(Oc3ccccc3)N3CCC[C@H]3C(=O)OCCCC)ccc2s1] | 542.3 [M + H]⁺ |
| allyl 5-(((((S)-1- butoxy-1-oxopropan- 2- yl)amino)(naphthalen- 1- yloxy)phosphoryl)meth- yl)benzo[b]thiophene- 2-carboxylate | 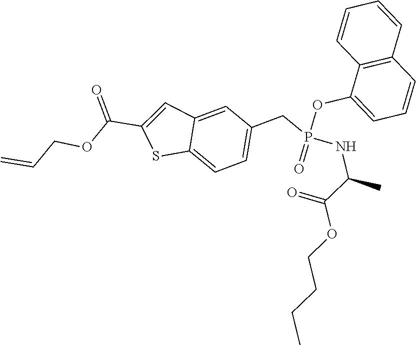 [SMILES: C=CCOC(=O)c1cc2cc(CP(=O)(N[C@@H](C)C(=O)OCCCC)Oc3cccc4ccccc34)ccc2s1] | 566.1 [M + H]⁺ |
| allyl 5-(((((S)-3- methyl-1-oxo-1- propoxybutan-2- yl)amino)(phenoxy)phos- phoryl)methyl)benzo [b]thiophene-2- carboxylate | 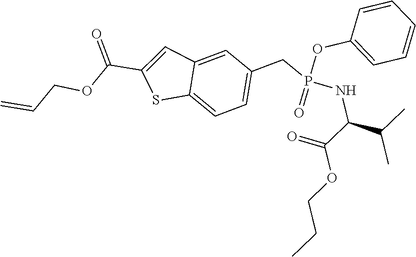 [SMILES: C=CCOC(=O)c1cc2cc(CP(=O)(N[C@H](C(=O)OCCC)C(C)C)Oc3ccccc3)ccc2s1] | ¹H NMR (400 MHz, CDCl3) δ 8.05 (d, J = 6.0 Hz, 1H), 7.92- 7.80 (m, 2H), 7.55-7.45 (m, 1H), 7.30-7.27 (m, 1H), 7.27- 7.24 (m, 1H), 7.15-7.08 (m, 3H), 6.13-5.99 (m, 1H), 5.45 (td, J = 1.2, 17.2 Hz, 1H), 5.36- 5.31 (m, 1H), 5.31 (s, 1H), 4.86 (d, J = 5.6 Hz, 2H), 4.03-3.90 (m, 1H), 3.86-3.75 (m, 1H), 3.50-3.39 (m, 2H), 3.21-3.03 (m, 1H), 1.94-1.83 (m, 1H), 1.59-1.50 (m, 2H), 0.91-0.86 (m, 3H), 0.85-0.78 (m, 3H), 0.67 (dd, J = 4.0, 6.8 Hz, 3H) |
| allyl 5-(((((S)-1-((R)- sec-butoxy)-1- oxopropan-2- yl)amino)(phenoxy)phos- phoryl)methyl) benzo[b]thiophene-2- carboxylate | 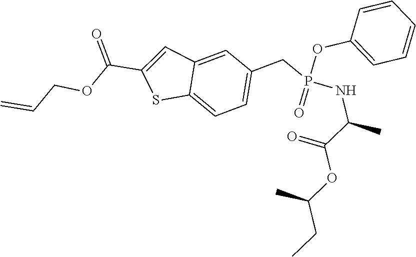 [SMILES: C=CCOC(=O)c1cc2cc(CP(=O)(N[C@@H](C)C(=O)O[C@H](C)CC)Oc3ccccc3)ccc2s1] | ¹H NMR (400 MHz, CDCl3) δ 8.04 (d, J = 6.4 Hz, 1H), 7.90- 7.79 (m, 2H), 7.48 (m, 1H), 7.30- 7.27 (m, 1H), 7.25 (d, J = 2.0 Hz, 1H), 7.17-7.09 (m, 3H), 6.09-5.99 (m, 1H), 5.47-5.40 (m, 1H), 5.33-5.29 (m, 1H), 4.85 (d, J = 5.6 Hz, 2H), 4.81- 4.71 (m, 1H), 4.01-3.90 (m, 1H), 3.48-3.38 (m, 2H), 1.55- 1.43 (m, 2H), 1.18-1.14 (m, 3H), 1.14-1.09 (m 3H), 0.83 (s, 3H) |
| allyl 5-(((((S)-1-oxo-1- propoxypropan-2- yl)amino)(2,2,2- trifluoroethoxy)phospho- ryl)methyl)benzo[b]thio- phene-2- carboxylate | 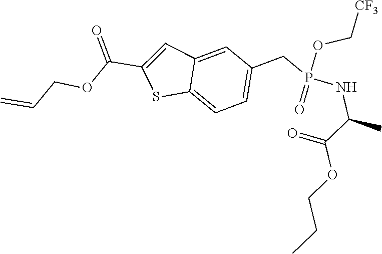 [SMILES: C=CCOC(=O)c1cc2cc(CP(=O)(N[C@@H](C)C(=O)OCCC)OCC(F)(F)F)ccc2s1] | ¹H NMR (400 MHz, CDCl₃) δ 8.08-8.01 (m, 1H), 7.86-7.79 (m, 2H), 7.47-7.37 (m, 1H), 6.12-5.98 (m, 1H), 5.45 (dd, J = 1.6, 17.2 Hz, 1H), 5.33 (dd, J = 1.2, 10.4 Hz, 1H), 4.86 (d, J = 5.6 Hz, 2H), 4.33-4.16 (m, 2H), 4.10-3.93 (m, 2H), 3.43-3.31 (m, 2H), 3.21-2.96 (m, 1H), 1.66-1.54 (m, 3H), 1.35 (d, J = 7.2 Hz, 2H), 1.26 (d, J = 7.2 Hz, 1H), 0.95-0.88 (m, 3H) |
| allyl 5-(((((S)-1- ethoxy-1-oxo-3- phenylpropan-2- yl)amino)(phenoxy)phos- phoryl)methyl)benzo [b]thiophene-2- carboxylate | 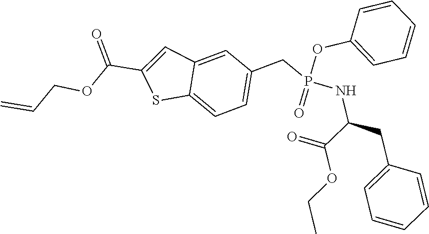 [SMILES: C=CCOC(=O)c1cc2cc(CP(=O)(N[C@@H](Cc3ccccc3)C(=O)OCC)Oc3ccccc3)ccc2s1] | 564 [M + H]⁺ |
| allyl 5-(((((S)-1- (benzyloxy)-4-methyl- 1-oxopentan-2- yl)amino)(phenoxy)phos- phoryl)methyl)benzo [b]thiophene-2- carboxylate | 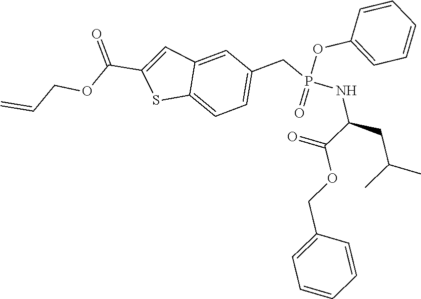 [SMILES: C=CCOC(=O)c1cc2cc(CP(=O)(N[C@@H](CC(C)C)C(=O)OCc3ccccc3)Oc3ccccc3)ccc2s1] | 592 [M + H]⁺ |
| allyl 5-(((((S)-1- (benzyloxy)-1- oxopropan-2- yl)amino)(phenoxy)phos- phoryl)methyl)benzo [b]thiophene-2- carboxylate | 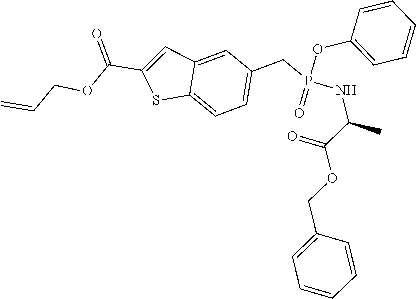 [SMILES: C=CCOC(=O)c1cc2cc(CP(=O)(N[C@@H](C)C(=O)OCc3ccccc3)Oc3ccccc3)ccc2s1] | 550 [M + H]⁺ |
| isopropyl (((2- ((allyloxy)carbonyl)ben- zo[b]thiophen-5- yl)methyl)(phenoxy)phos- phoryl)-L-prolinate | 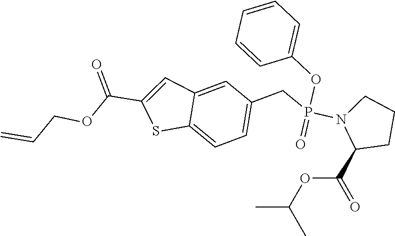 [SMILES: C=CCOC(=O)c1cc2cc(CP(=O)(Oc3ccccc3)N3CCC[C@H]3C(=O)OC(C)C)ccc2s1] | 528 [M + H]⁺ |
| allyl 5-(((((S)-2- oxoTHF-3- yl)amino)(phenoxy)phos- phoryl)methyl)benzo [b]thiophene-2- carboxylate | 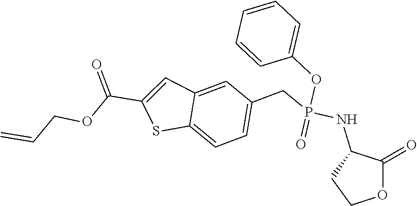 [SMILES: C=CCOC(=O)c1cc2cc(CP(=O)(N[C@H]3CCOC3=O)Oc3ccccc3)ccc2s1] | 472.1 [M + H]⁺ |
| allyl 5-(((((S)-1- (benzyloxy)-1-oxo-4- phenylbutan-2- yl)amino)(phenoxy)phos- phoryl)methyl)benzo [b]thiophene-2- carboxylate | 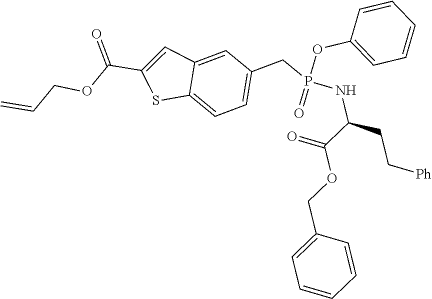 [SMILES: C=CCOC(=O)c1cc2cc(CP(=O)(N[C@@H](CCc3ccccc3)C(=O)OCc3ccccc3)Oc3ccccc3)ccc2s1] | 640 [M + H]⁺ |
| allyl 5-(((((S)-1- ethoxy-1-oxopropan- 2- yl)amino)(phenoxy)phos- phoryl)methyl)benzo [b]thiophene-2- carboxylate | 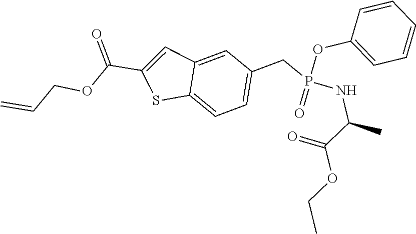 [SMILES: C=CCOC(=O)c1cc2cc(CP(=O)(N[C@@H](C)C(=O)OCC)Oc3ccccc3)ccc2s1] | 488 [M + H]⁺ |
| allyl 5-(((((S)-3- methoxy-1-oxo-1- propoxypropan-2- yl)amino)(phenoxy)phos- phoryl)methyl)benzo [b]thiophene-2- carboxylate | 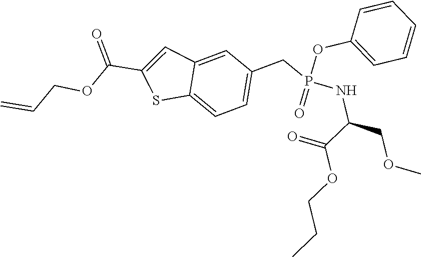 [SMILES: C=CCOC(=O)c1cc2cc(CP(=O)(N[C@@H](COC)C(=O)OCCC)Oc3ccccc3)ccc2s1] | 532 [M + H]⁺ |
| allyl 5-(((((S)-4- methoxy-1-oxo-1- propoxybutan-2- yl)amino)(phenoxy)phos- phoryl)methyl)benzo [b]thiophene-2- carboxylate | 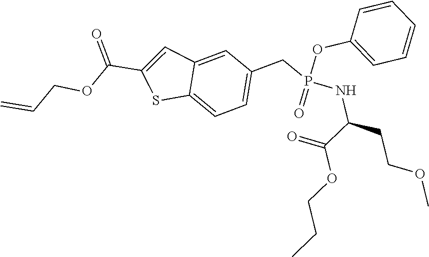 [SMILES: C=CCOC(=O)c1cc2cc(CP(=O)(N[C@@H](CCOC)C(=O)OCCC)Oc3ccccc3)ccc2s1] | 546 [M + H]⁺ |
| allyl 5-(((((S)-1-oxo-1- propoxy-3-(pyridin-2- yl)propan-2- yl)amino)(phenoxy)phos- phoryl)methyl)benzo [b]thiophene-2- carboxylate | 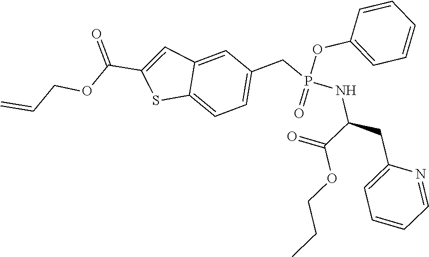 [SMILES: C=CCOC(=O)c1cc2cc(CP(=O)(N[C@@H](Cc3ccccn3)C(=O)OCCC)Oc3ccccc3)ccc2s1] | 579 [M + H]⁺ |
| allyl 5-((phenoxy((1- (propoxycarbonyl)cyclo- propyl)amino)phospho- ryl)methyl)benzo[b] thiophene-2- carboxylate | 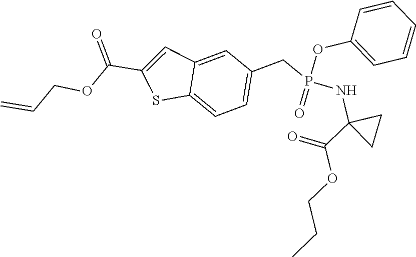 [SMILES: C=CCOC(=O)c1cc2cc(CP(=O)(NC3(C(=O)OCCC)CC3)Oc3ccccc3)ccc2s1] | 514.1 [M + H]⁺ |

### Step 2: Preparation of 5-(((2,6-dimethylphenoxy)(((S)-1-oxo-1-propoxypropan-2-yl)amino)phosphoryl)methyl)benzo[b]thiophene-2-carboxylic acid

To a stirred solution of allyl 5-(((2,6-dimethylphenoxy)(((S)-1-oxo-1-propoxypropan-2-yl)amino)phosphoryl)methyl)benzo[b]thiophene-2-carboxylate (50 mg, 94.4 μmol, 1 eq) in THF (4 mL) were added morpholine (40.6 μL, 472 μmol, 5 eq) and Pd(PPh₃)₄ (3.27 mg, 2.83 μmol, 0.03 eq) under nitrogen. The reaction mixture was stirred at room temperature 1 h. The solvent was removed under reduced pressure and the product was directly purified by reverse phase chromatography on a 100 g Cis cartridge eluting with a gradient of 5-80% acetonitrile in water (with 0.1% formic acid) to afford 5-(((2,6-dimethylphenoxy)(((S)-1-oxo-1-propoxypropan-2-yl)amino)phosphoryl)methyl)benzo[b]thiophene-2-carboxylic acid (36.0 mg, 77.9%) as a pale yellow solid. ¹H NMR (400 MHz, CDCl₃) δ 7.93-7.84 (m, 1H), 7.83-7.71 (m, 2H), 7.48-7.36 (m, 1H), 7.08-6.93 (m, 3H), 4.17-3.98 (m, 3H), 3.59-3.42 (m, 2H), 2.37 (s, 3H), 2.30 (s, 3H), 1.65-1.57 (m, 2H), 1.27-1.22 (m, 3H), 1.15-1.11 (m, 1H), 0.94-0.88 (m, 3H).

The intermediates in the table below were prepared using the method described above in Step 2 for the preparation of 5-(((2,6-dimethylphenoxy)(((S)-1-oxo-1-propoxypropan-2-yl)amino)phosphoryl)methyl)benzo[b]thiophene-2-carboxylic acid and utilizing the appropriate starting materials and modifications.

| Name | Structure | NMR; LCMS |
|---|---|---|
| 5-((((2-methyl-3-oxo- 3- propoxypropyl)amino) (phenoxy)phosphoryl) methyl)benzo[b]thiophene- 2-carboxylic acid | 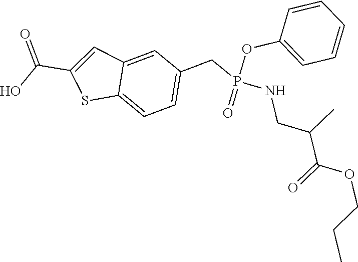 [SMILES: CCCOC(=O)C(C)CNP(=O)(Cc1ccc2sc(C(=O)O)cc2c1)Oc1ccccc1] | ¹H NMR (400 MHz, DMSO-d₆) δ 13.39 (br. s., 1H), 8.10-8.05 (m, 1H), 8.01-7.88 (m, 2H), 7.51- 7.44 (m, 1H), 7.39-7.28 (m, 2H), 7.22-7.05 (m, 3H), 5.22- 5.10 (m, 1H), 3.96-3.82 (m, 2H), 3.47-3.36 (m, 2H), 3.10-2.94 (m, 1H), 2.86-2.70 (m, 1H), 2.36- 2.22 (m, 1H), 1.56-1.45 (m, 2H), 0.90 (dd, J = 10.3, 7.1 Hz, 3H), 0.86-0.78 (m, 3H); 476.2 [M + H]⁺ |
| 5-((((S)-2- (butoxycarbonyl)-4,4- difluoropyrrolidin-1- yl)(phenoxy)phosphor- yl)methyl)benzo[b]thi- ophene-2-carboxylic acid | 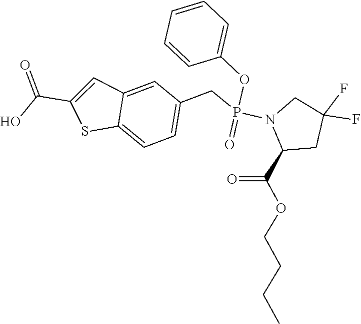 [SMILES: CCCCOC(=O)[C@@H]1CC(F)(F)CN1P(=O)(Cc1ccc2sc(C(=O)O)cc2c1)Oc1ccccc1] | ¹H NMR (400 MHz, DMSO-d₆) δ 13.49 (br. s., 1H), 8.14-8.06 (m, 1H), 8.04-7.91 (m, 2H), 7.55- 7.47 (m, 1H), 7.40-7.28 (m, 2H), 7.25-7.06 (m, 3H), 4.67- 4.56 (m, 0.4H), 4.35-4.28 (m, 0.6H), 4.08-3.69 (m, 3H), 3.68- 3.42 (m, 3H), 2.73-2.30 (m, 2H), 1.55-1.37 (m, 2H), 1.35-1.18 (m, 2H), 0.91-0.77 (m, 3H); 538.2 [M + H]⁺ |
| 5-(((((S)-1-oxo-1- propoxybutan-2- yl)amino)(phenoxy)phos- phoryl)methyl)benzo [b]thiophene-2- carboxylic acid | 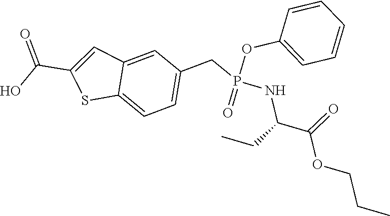 [SMILES: CCCOC(=O)[C@H](CC)NP(=O)(Cc1ccc2sc(C(=O)O)cc2c1)Oc1ccccc1] | ¹H NMR (400 MHz, CD₃OD) δ 8.01 (s, 1H), 7.97-7.83 (m, 2H), 7.54-7.47 (m, 1H), 7.36-7.25 (m, 2H), 7.21-7.11 (m, 3H), 3.95- 3.65 (m, 3H), 3.61-3.43 (m, 2H), 1.59-1.40 (m, 4H), 0.86 (q, J = 7.6 Hz, 3H), 0.73-0.63 (m, 3H); 476.2 [M + H]⁺ |
| 5-((((4-oxo-4- propoxybutan-2- yl)amino)(phenoxy)phos- phoryl)methyl)benzo [b]thiophene-2- carboxylic acid | 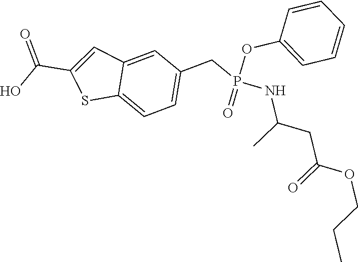 [SMILES: CCCOC(=O)CC(C)NP(=O)(Cc1ccc2sc(C(=O)O)cc2c1)Oc1ccccc1] | ¹H NMR (400 MHz, DMSO-d₆) δ 13.47 (br. s., 1H), 8.08 (s, 1H), 8.02-7.89 (m, 2H), 7.53-7.45 (m, 1H), 7.39-7.27 (m, 2H), 7.17- 7.09 (m, 3H), 5.22-5.04 (m, 1H), 3.92-3.80 (m, 2H), 3.61- 3.45 (m, 1H), 3.45-3.25 (m, 3H), 2.30-2.13 (m, 2H), 1.55-1.44 (m, 2H), 0.93-0.86 (m, 3H), 0.85- 0.79 (m, 3H); 476.2 [M + H]⁺ |
| 5-((((S)-2- (butoxycarbonyl)pyrro- lidin-1- yl)(phenoxy)phosphor- yl)methyl)benzo[b]thi- ophene-2-carboxylic acid | 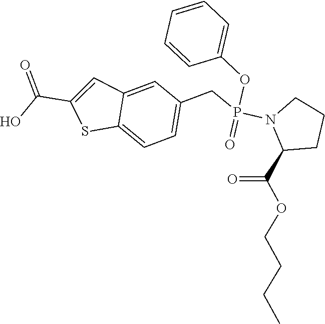 [SMILES: CCCCOC(=O)[C@@H]1CCCN1P(=O)(Cc1ccc2sc(C(=O)O)cc2c1)Oc1ccccc1] | 502.1 [M + H]⁺ |
| 5-(((((S)-1-butoxy-1- oxopropan-2- yl)amino)(naphthalen- 1- yloxy)phosphoryl)meth- yl)benzo[b]thiophene- 2-carboxylic acid | 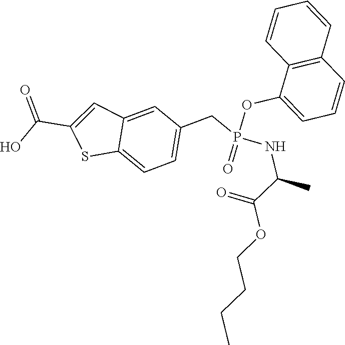 [SMILES: CCCCOC(=O)[C@H](C)NP(=O)(Cc1ccc2sc(C(=O)O)cc2c1)Oc1cccc2ccccc12] | 526.1 [M + H]⁺ |
| 5-(((((S)-3-methyl-1- oxo-1-propoxybutan- 2- yl)amino)(phenoxy)phos- phoryl)methyl)benzo [b]thiophene-2- carboxylic acid | 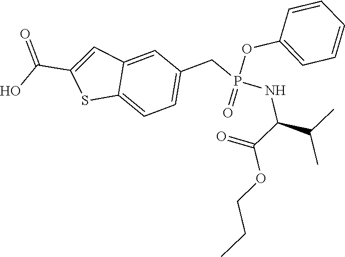 [SMILES: CCCOC(=O)[C@@H](NP(=O)(Cc1ccc2sc(C(=O)O)cc2c1)Oc1ccccc1)C(C)C] | 490.3 [M + H]⁺ |
| 5-(((((S)-1-((R)-sec- butoxy)-1-oxopropan- 2- yl)amino)(phenoxy)phos- phoryl)methyl)benzo [b]thiophene-2- carboxylic acid | 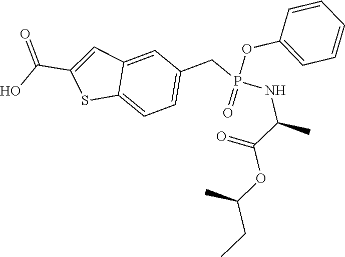 [SMILES: CC[C@@H](C)OC(=O)[C@H](C)NP(=O)(Cc1ccc2sc(C(=O)O)cc2c1)Oc1ccccc1] | 476.8 [M + H]⁺ |
| 5-(((((S)-1-oxo-1- propoxypropan-2- yl)amino)(2,2,2- trifluoroethoxy)phospho- ryl)methyl)benzo[b]thi- ophene-2-carboxylic acid | 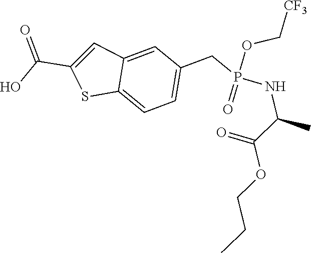 [SMILES: CCCOC(=O)[C@H](C)NP(=O)(Cc1ccc2sc(C(=O)O)cc2c1)OCC(F)(F)F] | ¹H NMR (400 MHz, CDCl₃) δ 7.83 (s, 1H), 7.81-7.75 (m, 2H), 7.32 (dd, J = 2.0, 8.4 Hz, 1H), 4.50-4.29 (m, 2H), 4.18-3.98 (m, 2H), 3.87-3.62 (m, 1H), 3.46- 3.32 (m, 2H), 1.72-1.64 (m, 2H), 1.42 (dd, J = 7.2, 17.2 Hz, 3H), 0.96 (q, J = 7.2 Hz, 3H) |
| 5-(((((S)-1-ethoxy-1- oxo-3-phenylpropan- 2- yl)amino)(phenoxy)phos- phoryl)methyl)benzo [b]thiophene-2- carboxylic acid | 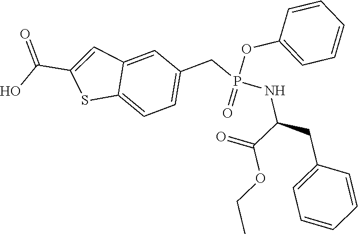 [SMILES: CCOC(=O)[C@H](Cc1ccccc1)NP(=O)(Cc1ccc2sc(C(=O)O)cc2c1)Oc1ccccc1] | 524 [M + H]⁺ |
| 5-(((((S)-1- (benzyloxy)-4-methyl- 1-oxopentan-2- yl)amino)(phenoxy)phos- phoryl)methyl)benzo [b]thiophene-2- carboxylic acid | 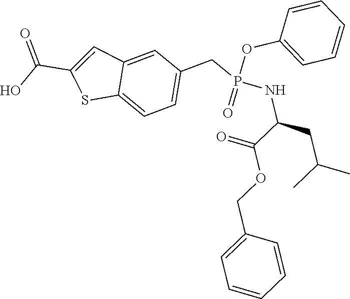 [SMILES: CC(C)C[C@H](NP(=O)(Cc1ccc2sc(C(=O)O)cc2c1)Oc1ccccc1)C(=O)OCc1ccccc1] | 552 [M + H]⁺ |
| 5-(((((S)-1- (benzyloxy)-1- oxopropan-2- yl)amino)(phenoxy)phos- phoryl)methyl)benzo [b]thiophene-2- carboxylic acid | 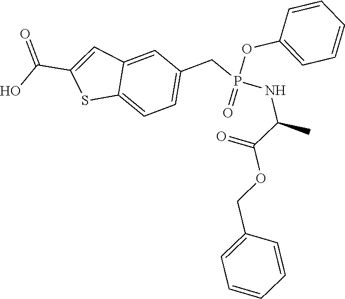 [SMILES: C[C@H](NP(=O)(Cc1ccc2sc(C(=O)O)cc2c1)Oc1ccccc1)C(=O)OCc1ccccc1] | 510 [M + H]⁺ |
| 5-((((S)-2- (isopropoxycarbonyl)pyr- rolidin-1- yl)(phenoxy)phosphor- yl)methyl)benzo[b]thi- ophene-2-carboxylic acid | 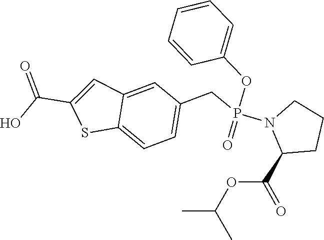 [SMILES: CC(C)OC(=O)[C@@H]1CCCN1P(=O)(Cc1ccc2sc(C(=O)O)cc2c1)Oc1ccccc1] | 488 [M + H]⁺ |
| 5-(((((S)-2-oxoTHF-3- yl)amino)(phenoxy)phos- phoryl)methyl)benzo [b]thiophene-2- carboxylic acid | 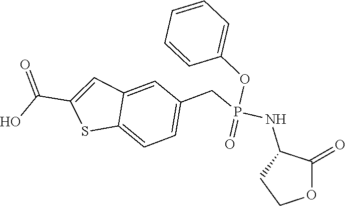 [SMILES: O=C(O)c1cc2cc(CP(=O)(N[C@H]3CCOC3=O)Oc3ccccc3)ccc2s1] | 432 [M + H]⁺ |
| 5-(((((S)-1- (benzyloxy)-1-oxo-4- phenylbutan-2- yl)amino)(phenoxy)phos- phoryl)methyl)benzo [b]thiophene-2- carboxylic acid | 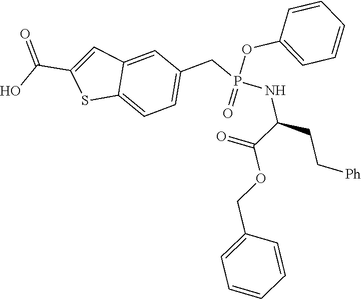 [SMILES: O=C(O)c1cc2cc(CP(=O)(N[C@@H](CCc3ccccc3)C(=O)OCc3ccccc3)Oc3ccccc3)ccc2s1] | 600 [M + H]⁺ |
| 5-(((((S)-1-ethoxy-1- oxopropan-2- yl)amino)(phenoxy)phos- phoryl)methyl)benzo [b]thiophene-2- carboxylic acid | 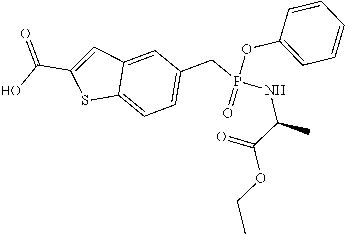 [SMILES: CCOC(=O)[C@H](C)NP(=O)(Cc1ccc2sc(C(=O)O)cc2c1)Oc1ccccc1] | 448 [M + H]⁺ |
| 5-(((((S)-3-methoxy-1- oxo-1-propoxypropan- 2- yl)amino)(phenoxy)phos- phoryl)methyl)benzo [b]thiophene-2- carboxylic acid | 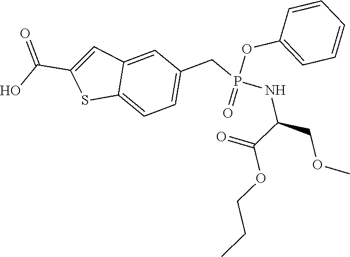 [SMILES: CCCOC(=O)[C@H](COC)NP(=O)(Cc1ccc2sc(C(=O)O)cc2c1)Oc1ccccc1] | 492 [M + H]⁺ |
| 5-(((((S)-4-methoxy-1- oxo-1-propoxybutan- 2- yl)amino)(phenoxy)phos- phoryl)methyl)benzo [b]thiophene-2- carboxylic acid | 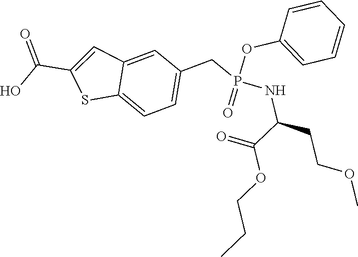 | 546 [M + H]⁺ |
| 5-(((((S)-1-oxo-1- propoxy-3-(pyridin-2- yl)propan-2- yl)amino)(phenoxy)phos- phoryl)methyl)benzo [b]thiophene-2- carboxylic acid | 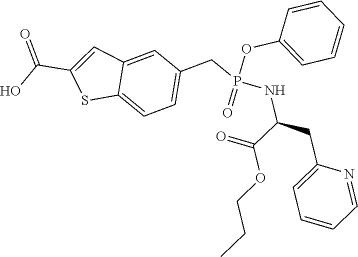 [SMILES: CCCOC(=O)[C@H](Cc1ccccn1)NP(=O)(Cc1ccc2sc(C(=O)O)cc2c1)Oc1ccccc1] | 539 [M + H]⁺ |
| 5-((phenoxy((1- (propoxycarbonyl)cyclo- propyl)amino)phospho- ryl)methyl)benzo[b]thio- phene-2-carboxylic acid | 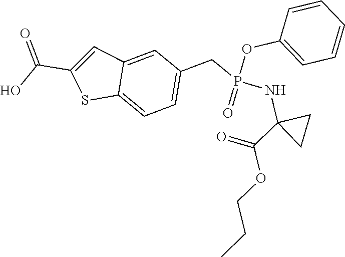 [SMILES: CCCOC(=O)C1(NP(=O)(Cc2ccc3sc(C(=O)O)cc3c2)Oc2ccccc2)CC1] | 474.0 [M + H]⁺ |

### Synthesis of 5-((morpholino(((S)-1-oxo-1-propoxypropan-2-yl)amino)phosphoryl)methyl)benzo[b]thiophene-2-carboxylic acid

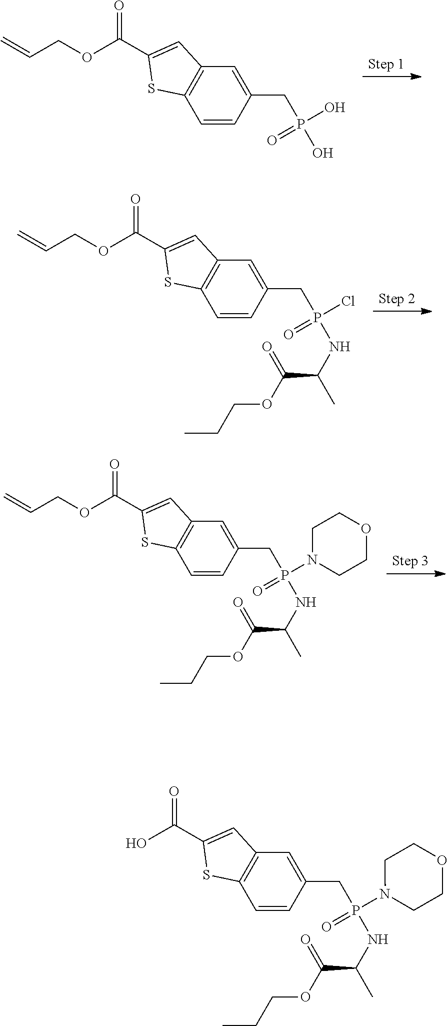 [SMILES: C=CCOC(=O)c1cc2cc(CP(=O)(O)O)ccc2s1 ; C=CCOC(=O)c1cc2cc(CP(=O)(Cl)N[C@@H](C)C(=O)OCCC)ccc2s1 ; C=CCOC(=O)c1cc2cc(CP(=O)(N[C@@H](C)C(=O)OCCC)N3CCOCC3)ccc2s1 ; CCCOC(=O)[C@H](C)NP(=O)(Cc1ccc2sc(C(=O)O)cc2c1)N1CCOCC1]

### Step 1: Synthesis of allyl 5-((chloro(((S)-1-oxo-1-propoxypropan-2-yl)amino)phosphoryl)methyl)benzo[b]thiophene-2-carboxylate

Oxalyl chloride (10 mL) was added dropwise to the solution of ((2-((allyloxy)carbonyl)benzo[b]thiophen-5-yl)methyl)phosphonic acid (1 g, 3.20 mmol, 1.0 eq.) in dry DCM (20 mL) and DMF (1 drop) at 20° C. The reaction mixture was stirred at 25° C. for an additional 1 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure to afford allyl 5-((dichlorophosphoryl)methyl)benzo[b]thiophene-2-carboxylate (960 mg, 2.74 mmol, 86%). allyl 5-((dichlorophosphoryl)methyl)benzo[b]thiophene-2-carboxylate (100 mg, 251 μmol, 1.0 eq.) was re-dissolved in anhydrous DCM (2 mL), then added to a mixture of propyl L-alaninate (36 mg, 276 μmol, 1.1 eq.) and triethylamine (101 mg, 1.00 mmol, 4.0 eq.) in anhydrous DCM (2 mL) at 0° C. The reaction was allowed to warm to 25° C., and stirred for an additional 15 min. The reaction progress was monitored by LCMS, and after completion, the reaction mixture was used in next Step directly without further purification.

### Step 2: Synthesis of allyl 5-((morpholino(((S)-1-oxo-1-propoxypropan-2-yl)amino)phosphoryl)methyl)benzo[b]thiophene-2-carboxylate

Allyl 5-((chloro(((S)-1-oxo-1-propoxypropan-2-yl)amino)phosphoryl)methyl)benzo[b]thiophene-2-carboxylate (200 mg, 450 μmol, 1.0 eq.) as a solution in DCM (5 mL) was added to a solution of morpholine (196 mg, 2.25 mmol, 5 eq.) and triethylamine (TEA) (273 mg, 2.7 mmol, 6 eq.) in DCM (1 mL). The mixture was stirred at 25° C. for 15 min, at which time LCMS showed formation of product. The mixture was concentrated under reduced pressure. The residue was purified by Biotage® C18 column chromatography to give allyl 5-((morpholino(((S)-1-oxo-1-propoxypropan-2-yl)amino)phosphoryl)methyl)benzo[b]thiophene-2-carboxylate (82 mg, 0.17 mmol, 38%) as a white solid. LCMS (ESI): m/z=495 [M+H]⁺.

The intermediates in the table below were prepared using the method described above in Step 2 for the preparation of allyl 5-((morpholino(((S)-1-oxo-1-propoxypropan-2-yl)amino)phosphoryl)methyl)benzo[b]thiophene-2-carboxylate and utilizing the appropriate starting materials and modifications.

| Name | Structure | LCMS |
|---|---|---|
| allyl 5-(((2-methoxyethoxy)(((S)-1- oxo-1-propoxypropan-2- yl)amino)phosphoryl)methyl)benzo [b]thiophene-2-carboxylate | 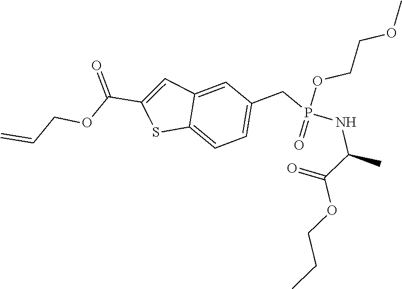 [SMILES: C=CCOC(=O)c1cc2cc(CP(=O)(N[C@@H](C)C(=O)OCCC)OCCOC)ccc2s1] | 484 [M + H]⁺ |
| allyl 5-((ethoxy(((S)-1-oxo-1- propoxypropan-2- yl)amino)phosphoryl)methyl)benzo [b]thiophene-2-carboxylate | 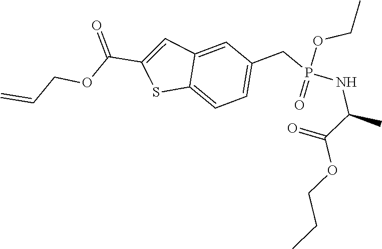 | 438 [M + H]⁺ |
| allyl 5-(((2,2- difluoropropoxy)(((S)-1-oxo-1- propoxypropan-2- yl)amino)phosphoryl)methyl)benzo [b]thiophene-2-carboxylate | 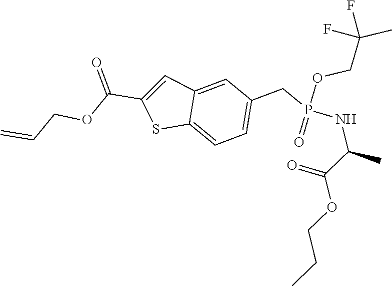 | 490 [M + H]⁺ |

### Step 3: Synthesis of 5-((morpholino(((S)-1-oxo-1-propoxypropan-2-yl)amino)phosphoryl)methyl)benzo[b]thiophene-2-carboxylic acid

To a solution of allyl 5-((morpholino(((S)-1-oxo-1-propoxypropan-2-yl)amino)phosphoryl)methyl)benzo[b]thiophene-2-carboxylate (82 mg, 0.17 mmol, 1.0 eq) in DCM (3 mL) were added Pd(PPh₃)₄ (20 mg, 17 μmol, 0.1 eq.) and pyrrolidine (12 mg, 0.17 mmol, 1.0 eq.). The mixture was purged and degassed with N₂ (three times), then stirred at 25° C. for 0.5 h. After completion, the mixture was concentrated under reduced pressure. The residue was purified by Biotage® C18 column chromatography to give 5-((morpholino(((S)-1-oxo-1-propoxypropan-2-yl)amino)phosphoryl)methyl)benzo[b]thiophene-2-carboxylic acid (50 mg, 0.11 mmol, 65%) as a white solid. LCMS (ESI): m/z=455 [M+H]⁺.

The intermediates in the table below were prepared using the method described above in Step 3 for the preparation of 5-((morpholino(((S)-1-oxo-1-propoxypropan-2-yl)amino)phosphoryl)methyl)benzo[b]thiophene-2-carboxylic acid and utilizing the appropriate starting materials and modifications.

| Name | Structure | LCMS |
|---|---|---|
| 5-(((2-methoxyethoxy)(((S)-1-oxo-1- propoxypropan-2- yl)amino)phosphoryl)methyl)benzo[b] thiophene-2-carboxylic acid | 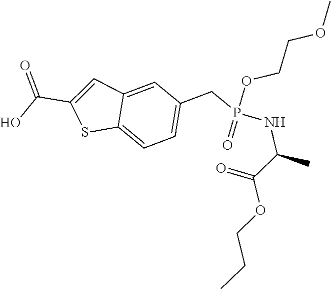 [SMILES: CCCOC(=O)[C@H](C)NP(=O)(Cc1ccc2sc(C(=O)O)cc2c1)OCCOC] | 444 [M + H]⁺ |
| 5-((ethoxy(((S)-1-oxo-1- propoxypropan-2- yl)amino)phosphoryl)methyl)benzo[b] thiophene-2-carboxylic acid | 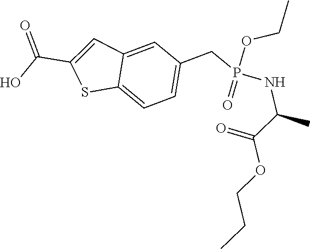 [SMILES: CCCOC(=O)[C@H](C)NP(=O)(Cc1ccc2sc(C(=O)O)cc2c1)OCC] | 414 [M + H]⁺ |
| 5-(((2,2-difluoropropoxy)(((S)-1-oxo- 1-propoxypropan-2- yl)amino)phosphoryl)methyl)benzo[b] thiophene-2-carboxylic acid | 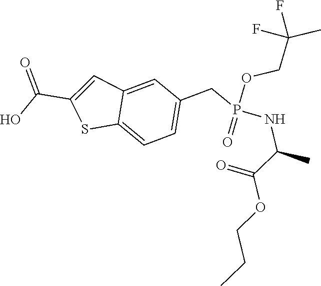 | 450 [M + H]⁺ |

### Synthesis of 5-(((((S)-4-methoxy-1-oxo-1-propoxybutan-2-yl)amino)(phenoxy)phosphoryl)methyl)benzo[b]thiophene-2-carboxylic acid

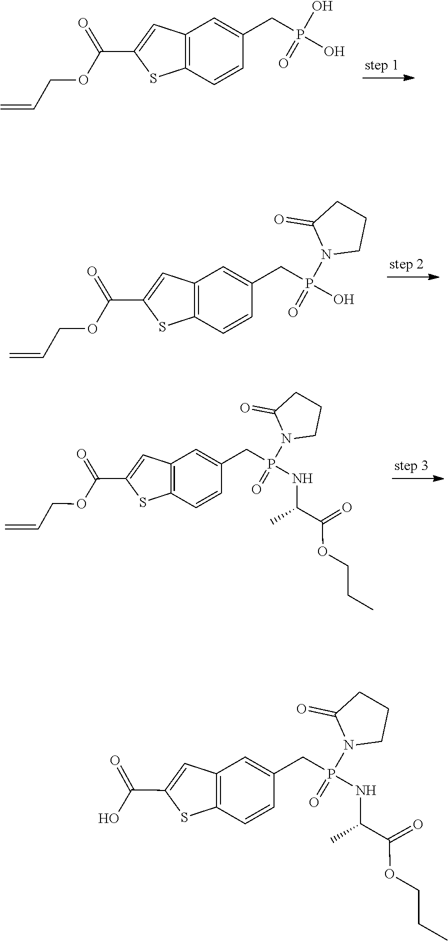 [SMILES: C=CCOC(=O)c1cc2cc(CP(=O)(O)O)ccc2s1 ; C=CCOC(=O)c1cc2cc(CP(=O)(O)N3CCCC3=O)ccc2s1 ; C=CCOC(=O)c1cc2cc(CP(=O)(N[C@@H](C)C(=O)OCCC)N3CCCC3=O)ccc2s1 ; CCCOC(=O)[C@H](C)NP(=O)(Cc1ccc2sc(C(=O)O)cc2c1)N1CCCC1=O]

### Step 1: Preparation of ((2-((allyloxy)carbonyl)benzo[b]thiophen-5-yl)methyl)(2-oxopyrrolidin-1-yl)phosphinic acid

Oxalyl chloride (10 mL) was added dropwise to the solution of ((2-((allyloxy)carbonyl)benzo[b]thiophen-5-yl)methyl)phosphonic acid (300 mg, 960 μmol, 1 eq.) in dry DCM (20 mL) and DMF (0.1 mL) at 0° C. The reaction mixture was stirred at 40° C. for an additional 1 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure to afford allyl 5-((dichlorophosphoryl)methyl)benzo[b]thiophene-2-carboxylate (300 mg). The residue (300 mg, 859 μmol, 1 eq.) was re-dissolved in anhydrous DCM (5 mL), then added to a mixture of pyrrolidin-2-one (218 mg, 2.57 mmol, 3 eq.) and TEA (259 mg, 2.57 mmol, 3 eq.) in anhydrous DCM (10 mL) at 0° C. The reaction was allowed to warm to 25° C. and stirred for an additional 2 h. After completion, the reaction mixture was concentrated under reduced pressure. The residue was purified by Biotage® C18 column chromatography to give ((2-((allyloxy)carbonyl)benzo[b]thiophen-5-yl)methyl)(2-oxopyrrolidin-1-yl)phosphinic acid (100 mg, 263 μmol, 31%) as a colorless oil. LCMS (ESI): m/z=380 [M+H]⁺.

### Step 2: Preparation of allyl 5-(((((S)-1-oxo-1-propoxypropan-2-yl)amino)(2-oxopyrrolidin-1-yl)phosphoryl)methyl)benzo[b]thiophene-2-carboxylate

Oxalyl chloride (201 mg, 1.59 mmol, 5 eq.) was added dropwise to the solution of ((2-((allyloxy)carbonyl)benzo[b]thiophen-5-yl)methyl)(2-oxopyrrolidin-1-yl)phosphinic acid (100 mg, 320 μmol, 1 eq.) in dry DCM (5 mL) and DMF (0.1 mL) at 0° C. The reaction mixture was stirred at 40° C. for an additional 1 hr. The reaction was monitored by pipetting out a small amount of crude sample and quenching it with MeOH to ensure mono-Cl phosphoryl chloride had been formed completely (mono-methoxy phosphonate was observed by LCMS). After completion, the excess oxalyl chloride and solvent were removed under reduced pressure to afford allyl 5-((chloro(2-oxopyrrolidin-1-yl)phosphoryl)methyl)benzo[b]thiophene-2-carboxylate (100 mg). The residue (100 mg, 251 μmol, 1 eq.) was re-dissolved in anhydrous DCM (10 mL), then added to a mixture of propyl L-alaninate (65.8 mg, 502 μmol, 2 eq.) and TEA (101 mg, 1.00 mmol, 4 eq.) in anhydrous DCM (10 mL) at 0° C. The reaction was allowed to warm to 25° C. and stirred for an additional 2 h. After completion, the reaction mixture was concentrated under reduced pressure. The residue was purified by Biotage® C18 column chromatography to give allyl 5-(((((S)-1-oxo-1-propoxypropan-2-yl)amino)(2-oxopyrrolidin-1-yl)phosphoryl)methyl)benzo[b]thiophene-2-carboxylate (23.0 mg, 46.6 μmol, 19%) as a colorless oil. LCMS (ESI): m/z=493 [M+H]⁺.

### Step 3: Preparation of 5-(((((S)-1-oxo-1-propoxypropan-2-yl)amino)(2-oxopyrrolidin-1-yl)phosphoryl)methyl)benzo[b]thiophene-2-carboxylic acid

To a solution of allyl 5-(((((S)-1-oxo-1-propoxypropan-2-yl)amino)(2-oxopyrrolidin-1-yl)phosphoryl)methyl)benzo[b]thiophene-2-carboxylate (22 mg, 44.6 μmol, 1 eq) in DCM (2 mL) were added Pd(PPh₃)₄ (5 mg, 4.46 μmol, 0.1 eq.) and pyrrolidine (3 mg, 44.6 μmol, 1.0 eq.). The mixture was purged and degassed with N₂ (three times), then stirred at 25° C. for 0.5 h. After completion, the mixture was quenched with HCl aqueous solution (1 N) and extracted with DCM (10 mL×3). The organic layers were combined and washed with brine (15 mL), dried over anhydrous Na₂SO₄, and concentrated under reduced pressure. The residue was purified by Biotage® C18 column chromatography to give 5-(((((S)-4-methoxy-1-oxo-1-propoxybutan-2-yl)amino)(phenoxy)phosphoryl)methyl)benzo[b]thiophene-2-carboxylic acid (13.0 mg, 28.7 μmol, 65%) as a colorless oil. LCMS (ESI): m/z=453 [M+H]⁺.

### Synthesis of 5-((bis((S)-2-(propoxycarbonyl)pyrrolidin-1-yl)phosphoryl)methyl)benzo[b]thiophene-2-carboxylic acid

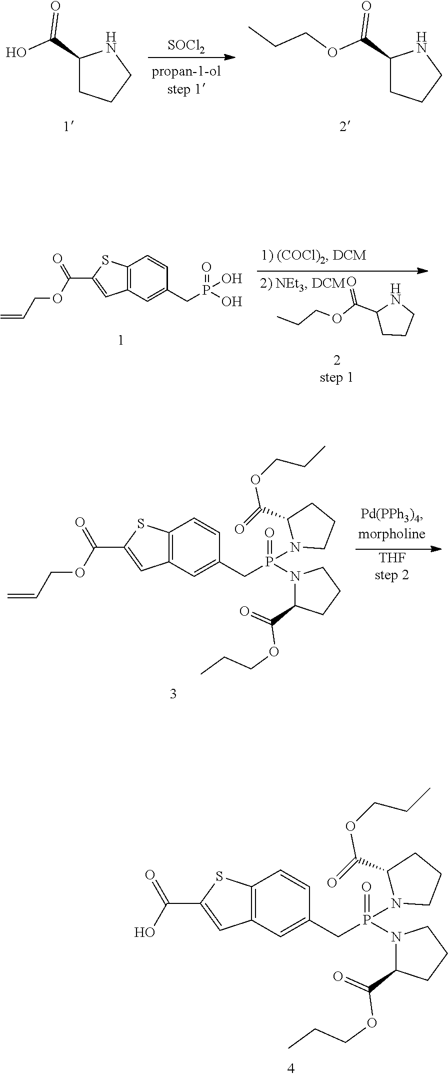

### Step 1: Preparation of propyl L-prolinate

To a solution of (2S)-pyrrolidine-2-carboxylic acid (2.00 g, 17.3 mmol, 1 eq) in propan-1-ol (13.0 mL, 266 mmol) at −78° C. was added dropwise thionyl chloride (3.76 mL, 51.9 mmol, 3 eq). The mixture was allowed to warm to room temperature and then heated at 80° C. overnight. The reaction mixture was concentrated under reduced pressure and the crude propyl L-prolinate (2.62 g, 16.6 mmol, 96.6%) was isolated as an orange sticky oil that was used directly in the next Step. LCMS (ESI): m/z=158.1 [M+H]⁺.

### Step 2: Preparation of dipropyl (((2-((allyloxy)carbonyl)benzo[b]thiophen-5-yl)methyl)phosphoryl)(S)-di-L-prolinate

To a solution of ((2-((allyloxy)carbonyl)benzo[b]thiophen-5-yl)methyl)phosphonic acid (300 mg, 0.9606 mmol, 1 eq) in DCM (10 mL) at 0° C. were added 2 drops of DMF (cat) and oxalyl chloride (246 μL, 2.88 mmol, 3 eq). The solution was stirred at 0° C. and was allowed to warm slowly to room temperature overnight. The reaction was concentrated under reduced pressure and then dried under high vacuum during 30 min. The crude residue was diluted in DCM (10 mL, dried with Na₂SO₄) and cooled down to 0° C. A solution of propyl L-prolinate (603 mg, 3.84 mmol, 4 eq) and triethylamine (668 μL, 4.80 mmol, 5 eq) in DCM (3 mL, dried with Na₂SO₄) was added and the mixture was stirred at room temperature for 3 d. The reaction was concentrated under reduced pressure and the crude residue was purified by reverse phase chromatography on a 150 g Cis cartridge eluting with 5-100% MeCN in water (with 0.1% formic acid) to give propyl dipropyl (((2-((allyloxy)carbonyl)benzo[b]thiophen-5-yl)methyl)phosphoryl)(S)-di-L-prolinate (274 mg, 48.3%) as an orange oil. LCMS (ESI): m/z=591.2 [M+H]⁺.

The intermediates in the table below were prepared using the method described above in Step 2 for the preparation of dipropyl (((2-((allyloxy)carbonyl)benzo[b]thiophen-5-yl)methyl)phosphoryl)(S)-di-L-prolinate and utilizing the appropriate starting materials and modifications.

| Name | Structure | LCMS |
|---|---|---|
| dibutyl 2,2′-((((2- ((allyloxy)carbonyl)benzo[b]thiophen-5- yl)methyl)phosphoryl)bis(azanediyl))(2S, 2′S)-dipropionate | 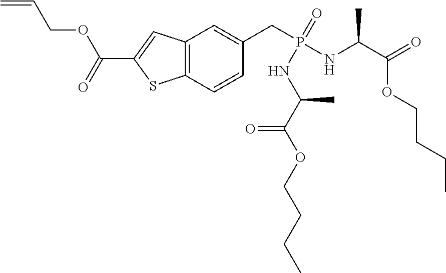 [SMILES: C=CCOC(=O)c1cc2cc(CP(=O)(N[C@@H](C)C(=O)OCCCC)N[C@@H](C)C(=O)OCCCC)ccc2s1] | 567 [M + H]⁺ |

### Step 3: Preparation of 5-((bis((S)-2-(propoxycarbonyl)pyrrolidin-1-yl)phosphoryl)methyl)benzo[b]thiophene-2-carboxylic acid

To a solution of propyl dipropyl (((2-((allyloxy)carbonyl)benzo[b]thiophen-5-yl)methyl)phosphoryl)(S)-di-L-prolinate (274 mg, 0.4638 mmol, 1 eq) in THF (30 mL) were added morpholine (199 μL, 2.31 mmol, 5 eq) and Pd(PPh₃)₄ (53.5 mg, 0.04638 mmol, 0.10 eq). The reaction mixture was stirred at room temperature for 2 h and THF was removed under reduced pressure. The crude residue was purified by reverse phase chromatography on a 100 g Cis cartridge eluting with 5-100% MeCN in water (with 0.1% formic acid) to give 5-((bis((S)-2-(propoxycarbonyl)pyrrolidin-1-yl)phosphoryl)methyl)benzo[b]thiophene-2-carboxylic acid (203 mg, 79.6%) as a white solid. LCMS (ESI): m/z=551.2 [M+H]⁺.

The intermediates in the table below were prepared using the method described above in Step 2 for the preparation of 5-((bis((S)-2-(propoxycarbonyl)pyrrolidin-1-yl)phosphoryl)methyl)benzo[b]thiophene-2-carboxylic acid and utilizing the appropriate starting materials and modifications.

| Name | Structure | LCMS |
|---|---|---|
| 5-((bis(((S)-1-butoxy-1-oxopropan-2- yl)amino)phosphoryl)methyl)benzo[b]thi- ophene-2-carboxylic acid | 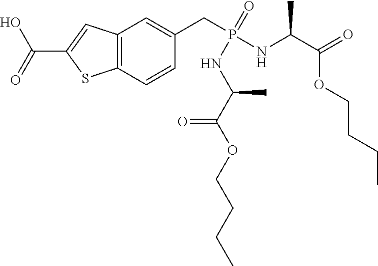 [SMILES: CCCCOC(=O)[C@H](C)NP(=O)(Cc1ccc2sc(C(=O)O)cc2c1)N[C@@H](C)C(=O)OCCCC] | 527 [M + H]⁺ |

### Synthesis of 5-(((((S)-1-isopropoxy-3-methoxy-1-oxopropan-2-yl)amino)(phenoxy)phosphoryl)methyl)benzo[b]thiophene-2-carboxylic acid

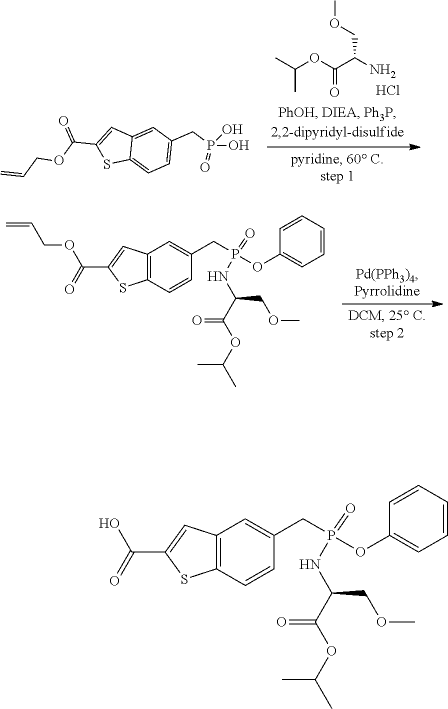 [SMILES: COC[C@H](N)C(=O)OC(C)C ; Cl ; C=CCOC(=O)c1cc2cc(CP(=O)(O)O)ccc2s1 ; C=CCOC(=O)c1cc2cc(CP(=O)(N[C@@H](COC)C(=O)OC(C)C)Oc3ccccc3)ccc2s1 ; COC[C@H](NP(=O)(Cc1ccc2sc(C(=O)O)cc2c1)Oc1ccccc1)C(=O)OC(C)C]

### Step 1: Preparation of allyl 5-(((((S)-1-isopropoxy-3-methoxy-1-oxopropan-2-yl)amino)(phenoxy)phosphoryl)methyl)benzo[b]thiophene-2-carboxylate

To a solution of ((2-((allyloxy)carbonyl)benzo[b]thiophen-5-yl)methyl)phosphonic acid (49.3 mg, 158 μmol, 1 eq.), propan-2-yl (2S)-2-amino3-methoxypropanoate hydrochloride (31.2 mg, 158 μmol, 1 eq.) and phenol (19.3 mg, 206 μmol, 1.3 eq.) in pyridine (3 mL) were added N,N-diisopropylethylamine (162 mg, 1.26 mmol, 8 eq.), 2,2-dipyridyl-disulfide (140 mg, 635 mol, 4 eq.) and triphenylphosphine (166 mg, 635 mol, 4 eq.) at 25° C. The mixture was heated to 60° C. and stirred at 60° C. for 48 h under N₂ atmosphere. After completion, the reaction mixture was concentrated under reduced pressure. The residue was purified by Biotage® C18 column chromatography to afford allyl 5-(((((S)-1-isopropoxy-3-methoxy-1-oxopropan-2-yl)amino)(phenoxy)phosphoryl)methyl)benzo[b]thiophene-2-carboxylate (35.0 mg, 65.8 mol, 42%) as a yellow solid. LCMS (ESI): m/z=532 [M+H]⁺.

The intermediates in the table below were prepared using the method described above in Step 1 for the preparation of allyl 5-(((((S)-1-isopropoxy-3-methoxy-1-oxopropan-2-yl)amino)(phenoxy)phosphoryl)methyl)benzo[b]thiophene-2-carboxylate and utilizing the appropriate starting materials and modifications.

| Name | Structure | LCMS |
|---|---|---|
| allyl 5-((((1-isopropoxy-2-methyl-1- oxopropan-2- yl)amino)(phenoxy)phosphoryl)methyl)ben- zo[b]thiophene-2-carboxylate | 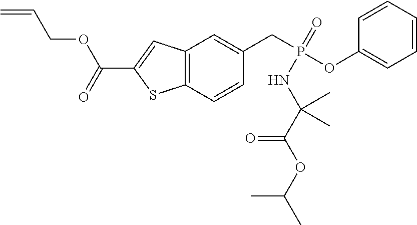 [SMILES: C=CCOC(=O)c1cc2cc(CP(=O)(NC(C)(C)C(=O)OC(C)C)Oc3ccccc3)ccc2s1] | 516.2 [M + H]⁺ |
| diisopropyl 2,2′-((((2- ((allyloxy)carbonyl)benzo[b]thiophen-5- yl)methyl)phosphoryl)bis(azanediyl))(2S, 2′S)-dipropionate | 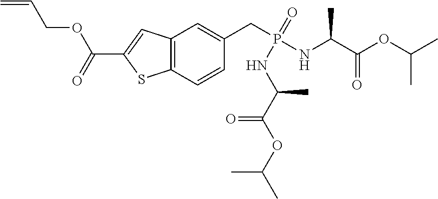 [SMILES: C=CCOC(=O)c1cc2cc(CP(=O)(N[C@@H](C)C(=O)OC(C)C)N[C@@H](C)C(=O)OC(C)C)ccc2s1] | 539 [M + H]⁺ |
| allyl 5-((((2-methyl-1-oxo-1- propoxypropan-2- yl)amino)(phenoxy)phosphoryl)methyl)ben- zo[b]thiophene-2-carboxylate | 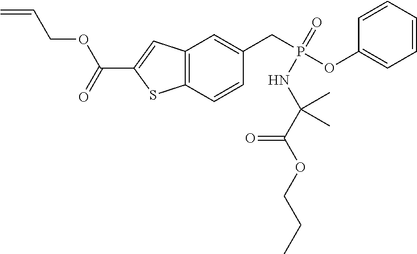 [SMILES: C=CCOC(=O)c1cc2cc(CP(=O)(NC(C)(C)C(=O)OCCC)Oc3ccccc3)ccc2s1] | 516.1 [M + H]⁺ |

### Step 2: Preparation of 5-(((((S)-1-isopropoxy-3-methoxy-1-oxopropan-2-yl)amino)(phenoxy)phosphoryl)methyl)benzo[b]thiophene-2-carboxylic acid

To a solution of allyl 5-(((((S)-1-isopropoxy-3-methoxy-1-oxopropan-2-yl)amino)(phenoxy)phosphoryl)methyl)benzo[b]thiophene-2-carboxylate (2, 150 mg, 282 μmol, 1 eq) in DCM (5 mL) were added Pd(PPh₃)₄ (65.1 mg, 56.4 μmol, 0.2 eq.) and pyrrolidine (20.0 mg, 282 μmol, 1.0 eq.). The mixture was purged and degassed with N₂ (three times), then stirred at 25° C. for 1 hr. After completion, the mixture was concentrated under reduced pressure. The residue was purified by Biotage® C18 column chromatography to give 5-(((((S)-1-isopropoxy-3-methoxy-1-oxopropan-2-yl)amino)(phenoxy)phosphoryl)methyl)benzo[b]thiophene-2-carboxylic acid (120 mg, 244 μmol, 87%) as a yellow solid. LCMS (ESI): m/z=492.1 [M+H]⁺.

The intermediates in the table below were prepared using the method described above in Step 2 for the preparation of 5-(((((S)-1-isopropoxy-3-methoxy-1-oxopropan-2-yl)amino)(phenoxy)phosphoryl)methyl)benzo[b]thiophene-2-carboxylic acid and utilizing the appropriate starting materials and modifications.

| Name | Structure | LCMS |
|---|---|---|
| 5-((((1-isopropoxy-2-methyl-1- oxopropan-2- yl)amino)(phenoxy)phosphoryl)methyl) benzo[b]thiophene-2-carboxylic acid | 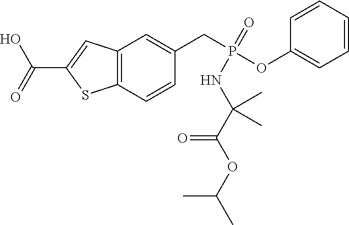 [SMILES: CC(C)OC(=O)C(C)(C)NP(=O)(Cc1ccc2sc(C(=O)O)cc2c1)Oc1ccccc1] | 476.5 [M + H]⁺ |
| 5-((bis(((S)-1-isopropoxy-1- oxopropan-2- yl)amino)phosphoryl)methyl)benzo[b]thio- phene-2-carboxylic acid | 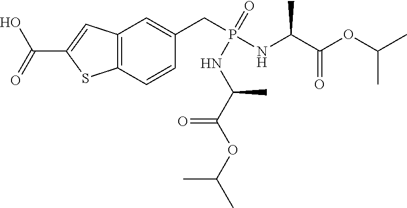 [SMILES: CC(C)OC(=O)[C@H](C)NP(=O)(Cc1ccc2sc(C(=O)O)cc2c1)N[C@@H](C)C(=O)OC(C)C] | 499 [M + H]⁺ |
| 5-((((2-methyl-1-oxo-1- propoxypropan-2- yl)amino)(phenoxy)phosphoryl)methyl) benzo[b]thiophene-2-carboxylic acid | 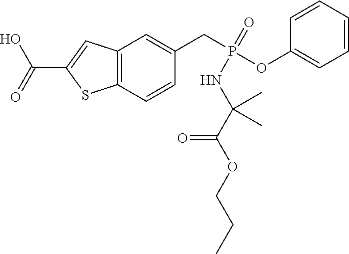 [SMILES: CCCOC(=O)C(C)(C)NP(=O)(Cc1ccc2sc(C(=O)O)cc2c1)Oc1ccccc1] | 476.2 [M + H]⁺ |

### Synthesis of 5-(1-fluoro-1-((((S)-1-oxo-1-propoxypropan-2-yl)amino)(phenoxy)phosphoryl)ethyl)benzo[b]thiophene-2-carboxylic acid

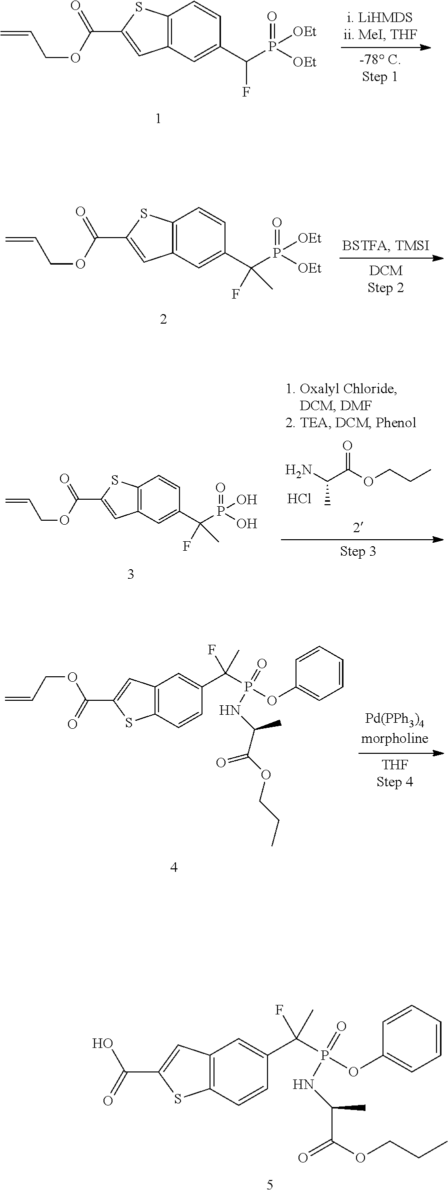

### Step 1: Preparation of allyl 5-(1-(diethoxyphosphoryl)-1-fluoroethyl)benzo[b]thiophene-2-carboxylate

To a solution of allyl 5-((diethoxyphosphoryl)fluoromethyl)benzo[b]thiophene-2-carboxylate (2.66 g, 6.88 mmol, 1 eq) in THF (50 mL) at −78° C. were added 1 M LiHMDS in THF (6.88 mL, 6.88 mmol, 1 eq) and methyl iodide (850 μL, 13.7 mmol, 2 eq) dropwise. The mixture was stirred at −78° C. for 5 min. The reaction was quenched with a saturated aqueous solution of ammonium chloride (30 mL), warmed up to room temperature and the product was extracted with EtOAc (2×100 mL). The combined organic extracts were dried with sodium sulfate, filtered and concentrated under reduced pressure. The crude material was purified by reverse phase chromatography on a 150 g Cis cartridge eluting with a gradient of 5-80% acetonitrile in water (with 0.1% formic acid). The fractions were combined and concentrated under reduced pressure to give allyl 5-(1-(diethoxyphosphoryl)-1-fluoroethyl)benzo[b]thiophene-2-carboxylate (1.44 g, 52.3%) as a brown oil. LCMS (ESI) m/z=401.2

### Step 2: Preparation of (1-(2-((allyloxy)carbonyl)benzo[b]thiophen-5-yl)-1-fluoroethyl)phosphonic acid

To a solution of allyl 5-(1-(diethoxyphosphoryl)-1-fluoroethyl)benzo[b]thiophene-2-carboxylate (1.44 g, 3.59 mmol, 1 eq) in DCM (30 mL) at 0° C. were added N,O-bis(trimethylsilyl)trifluoroacetamide (4.74 mL, 17.9 mmol) followed by slow addition of trimethylsilyl iodide (1.52 mL, 10.7 mmol, 3 eq) in DCM (2 mL). The solution was stirred at 0° C. for 1 h. The reaction was quenched with the addition of a 2:1 solution of water and acetonitrile (with 0.1% TFA) (2 mL). The solvent was removed under reduced pressure. The crude residue was purified by reverse phase chromatography on a 150 g Cis cartridge eluting with a gradient of 5-60% acetonitrile in water (with 0.1% formic acid). The combined fractions were concentrated and freeze-dried to give (1-(2-((allyloxy)carbonyl)benzo[b]thiophen-5-yl)-1-fluoroethyl)phosphonic acid (887 mg, 72.1%) as a pale-yellow solid. ¹H NMR (400 MHz, DMSO-d₆) 11.84-11.05 (m, 2H), 8.34-8.28 (m, 1H), 8.12-8.02 (m, 2H), 7.66-7.56 (m, 1H), 6.12-5.98 (m, 1H), 5.49-5.39 (m, 1H), 5.35-5.27 (m, 1H), 4.89-4.81 (m, 2H), 1.96-1.78 (m, 3H).

### Step 3: Preparation of allyl 5-(1-fluoro-1-((((S)-1-oxo-1-propoxypropan-2-yl)amino)(phenoxy)phosphoryl)ethyl)benzo[b]thiophene-2-carboxylate

To a suspension of (1-(2-((allyloxy)carbonyl)benzo[b]thiophen-5-yl)-1-fluoroethyl)phosphonic acid (160 mg, 464 μmol, 1 eq) and 1 drop of DMF (cat) in DCM (30 mL) at 0° C. was added oxalyl chloride (118 μL, 1.39 mmol, 3 eq) and the reaction was stirred for 20 h. The reaction was concentrated under reduced pressure and then dried under high vacuum during 10 min. The crude residue was diluted in 1,2-dichloroethane (3 mL) and a solution of phenol (43.6 mg, 464 μmol, 1 eq) and triethylamine (322 μL, 2.32 mmol, 5 eq) in 1,2-dichloroethane (3 mL, dried with Na₂SO₄) was added to the mixture which was stirred at 45° C. for 3 h. Propyl (2S)-2-aminopropanoate hydrochloride (77.7 mg, 464 μmol, 1 eq) in dichloromethane (10 mL, dried with Na₂SO₄) was added directly to the solution and stirred at room temperature overnight. The reaction mixture was quenched with water and the dichloromethane was removed in vacuo. The product was purified by reverse phase chromatography on a 50 g Cis cartridge eluting with a gradient of 5-100% acetonitrile in water (with 0.1% formic acid) to afford allyl 5-(1-fluoro-1-((((S)-1-oxo-1-propoxypropan-2-yl)amino)(phenoxy)phosphoryl)ethyl)benzo[b]thiophene-2-carboxylate (30.0 mg, 12%). ¹H NMR (400 MHz, CDCl₃) δ 8.12-8.02 (m, 2H), 7.93-7.82 (m, 1H), 7.72-7.64 (m, 1H), 7.38-7.28 (m, 1H), 7.25-7.13 (m, 2H), 7.09-7.00 (m, 1H), 7.00-6.92 (m, 1H), 6.13-5.98 (m, 1H), 5.45 (br d, J=17.1 Hz, 1H), 5.33 (br d, J=10.5 Hz, 1H), 4.86 (br d, J=4.9 Hz, 2H), 4.25-4.11 (m, 0.5H), 4.10-4.00 (m, 1H), 3.95-3.76 (m, 1H), 3.68-3.48 (m, 1.25H), 3.45-3.36 (m, 0.25H), 2.44-2.74 (m, 1H), 2.17-2.01 (m, 3H), 1.70-1.59 (m, 1H), 1.56-1.46 (m, 0.5H), 1.45-1.33 (m, 0.5H), 1.32-1.23 (m, 1.5H), 1.13-1.05 (m, 0.5H), 0.97-0.89 (m, 1.5H), 0.87-0.80 (m, 0.5H), 0.80-0.72 (m, 1H).

### Step 4: Preparation of 5-(1-fluoro-1-((((S)-1-oxo-1-propoxypropan-2-yl)amino)(phenoxy)phosphoryl)ethyl)benzo[b]thiophene-2-carboxylic acid

To a stirred solution of allyl 5-(1-fluoro-1-((((S)-1-oxo-1-propoxypropan-2-yl)amino)(phenoxy)phosphoryl)ethyl)benzo[b]thiophene-2-carboxylate (30 mg, 56.2 μmol, 1 eq) in THF (3 mL) were added morpholine (24.1 μL, 281 μmol, 5 eq) and Pd(PPh₃)₄ (3.24 mg, 2.81 μmol, 0.05 eq) under nitrogen. The reaction mixture was stirred at room temperature for 1 h. The reaction was concentrated and the crude was directly purified by reverse phase chromatography on a 50 g Cis cartridge eluting with a gradient of 5-100% acetonitrile in water (with 0.1% formic acid) to afford 5-(1-fluoro-1-((((S)-1-oxo-1-propoxypropan-2-yl)amino)(phenoxy)phosphoryl)ethyl)benzo[b]thiophene-2-carboxylic acid (30.0 mg, 108%) as a beige solid. LCMS (ESI) m/z=494.2 [M+H]⁺.

### Synthesis of perfluorophenyl 5-(((((S)-1-oxo-1-propoxypropan-2-yl)amino)(phenoxy)phosphoryl)methyl)benzo[b]thiophene-2-carboxylate

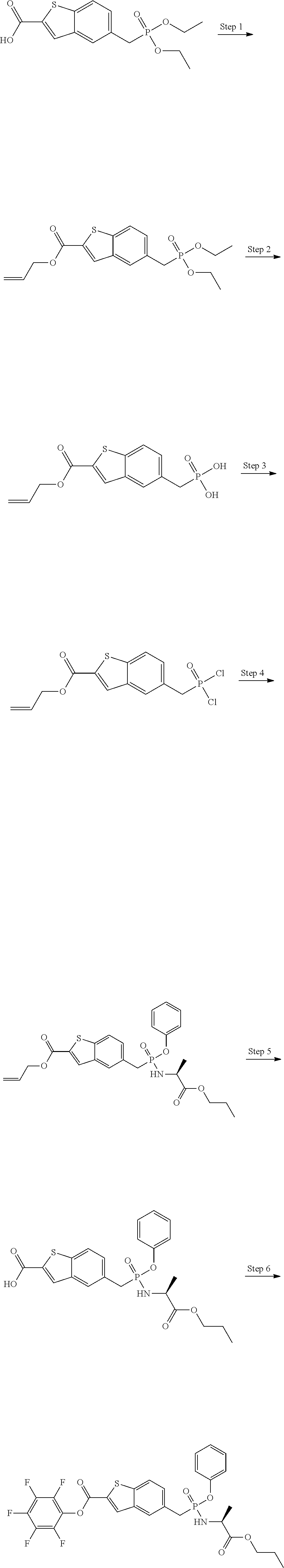

### Step 1: Preparation of allyl 5-((diethoxyphosphoryl)methyl)benzo[b]thiophene-2-carboxylate

To a solution of 5-((diethoxyphosphoryl)methyl)benzo[b]thiophene-2-carboxylic acid (25 g, 76 mmol, 1 eq) in DMF (250 mL) was added Na₂CO₃ (20 g, 190 mmol, 2.5 eq) and 3-bromoprop-1-ene (23 g, 190 mmol, 2.5 eq), the mixture was stirred at 25° C. for 12 h. The mixture was diluted with water (500 mL) and extracted with EtOAc (500 mL×3). The combined organic layers were washed with brine (500 mL×3), dried over anhydrous Na₂SO₄, filtered and concentrated under reduced pressure to give a residue. The residue was purified by flash silica gel chromatography to give allyl 5-((diethoxyphosphoryl)methyl)benzo[b]thiophene-2-carboxylate (25 g, 68 mmol, 89% yield) as a white solid. ¹H NMR (400 MHz, CDCl₃) δ 7.97 (s, 1H), 7.79-7.67 (m, 2H), 7.39-7.28 (m, 1H), 6.05-5.88 (m, 1H), 5.43-5.33 (m, 1H), 5.29-5.20 (m, 1H), 4.83-4.73 (m, 2H), 4.00-3.88 (m, 4H), 3.29-3.11 (m, 2H), 1.18 (d, J=6.4 Hz, 6H).

### Step 2: Preparation of ((2-((allyloxy)carbonyl)benzo[b]thiophen-5-yl)methyl)phosphonic acid

To a solution of allyl 5-((diethoxyphosphoryl)methyl)benzo[b]thiophene-2-carboxylate (25 g, 68 mmol, 1 eq) in DCM (250 mL) was added TMSI (54 g, 272 mmol, 4 eq) in a dropwise manner at 0° C., the mixture was stirred at 0° C. for 1 h. The reaction mixture was concentrated under reduced pressure to give a yellow oil. The oil was purified by reversed phase (TFA) to lyophilized to give ((2-((allyloxy)carbonyl)benzo[b]thiophen-5-yl)methyl)phosphonic acid (13 g, 42 mmol, 61% yield) as a white solid. LCMS (ESI) m/z=313.2 [M+H]⁺.

### Step 3: Preparation of allyl 5-((dichlorophosphoryl)methyl)benzo[b]thiophene-2-carboxylate

To a solution of ((2-((allyloxy)carbonyl)benzo[b]thiophen-5-yl)methyl)phosphonic acid (2.0 g, 6.4 mmol, 1 eq) in DCM (30 mL) was added DMF (0.09 mL) and was cooled to 0° C., then oxalic dichloride (2.4 g, 19.2 mmol, 3 eq) was dropwise, after that the reaction was warmed to 40° C. and was stirred 1 h to give a yellow clean solution. The reaction mixture was concentrated under reduced pressure to give allyl 5 ((dichlorophosphoryl)methyl)benzo[b]thiophene-2-carboxylate (2.0 g, crude) as a brassy yellow solid. LCMS (ESI) m/z=341.2 [M+H]⁺.

### Step 4: Preparation of allyl 5-(((((S)-1-oxo-1-propoxypropan-2-yl)amino)(phenoxy)phosphoryl)methyl)benzo[b]thiophene-2-carboxylate

To a solution of allyl 5-((dichlorophosphoryl)methyl)benzo[b]thiophene-2-carboxylate (2.0 g, crude, 1 eq) in DCM (40 mL) was cooled to 0° C. and was added a solution of phenol (0.43 g, 4.58 mmol, 1 eq) in DCM (8 mL), then ethylbis(propan-2-yl)amine (2.2 g, 17 mmol, 3 eq) in DCM (50 mL) was dropwise over 0.5 h. Then propyl L-alaninate hydrochloride (1.44 g, 1.58 mmol, 1.5 eq) in DCM (8 mL) was added, the mixture was stirred at 0° C. for 0.5 h. The reaction mixture was concentrated under reduced pressure to give a residue. The residue was purified by column chromatography to give allyl 5-(((((S)-1-oxo-1-propoxypropan-2-yl)amino)(phenoxy)phosphoryl)methyl)benzo[b]thiophene-2-carboxylate (1.4 g, 2.8 mmol, 43.8% yield) as a yellow oil. LCMS (ESI) m/z=502.2 [M+H]⁺.

### Step 5: Preparation of 5-(((((S)-1-oxo-1-propoxypropan-2-yl)amino)(phenoxy)phosphoryl)methyl)benzo[b]thiophene-2-carboxylic acid

To a solution of allyl 5-(((((S)-1-oxo-1-propoxypropan-2-yl)amino)(phenoxy)phosphoryl)methyl)benzo[b]thiophene-2-carboxylate (1.4 g, 2.8 mmol) in DCM (20 mL) was added Pd(PPh₃)₄ (0.3 g, 0.3 mmol, 0.1 eq) and pyrrolidine (0.4 g, 5.6 mmol, 2 eq). Then the reaction was stirred at 25° C. for 15 min to give a yellow clean solution. The reaction mixture was concentrated under reduced pressure to give a residue. The residue was purified by reversed phase (TFA) to freeze-drying to give 5-(((((S)-1-oxo-1-propoxypropan-2-yl)amino)(phenoxy)phosphoryl)methyl)benzo[b]thiophene-2-carboxylic acid (1.1 g, 2.4 mmol, 85.9% yield) as a yellow oil. LCMS (ESI) m/z=461.9 [M+H]⁺. ¹H NMR (400 MHz, CDCl₃) δ 7.91-7.86 (m, 1H), 7.84-7.76 (m, 2H), 7.45-7.38 (m, 1H), 7.38-7.31 (m, 2H), 7.27-7.15 (m, 3H), 4.28-3.95 (m, 3H), 3.56-3.42 (m, 2H), 1.64 (td, J=7.2, 14.0 Hz, 2H), 1.33-1.18 (m, 3H), 0.93 (dt, J=4.8, 7.2 Hz, 3H).

### Step 6: Preparation of perfluorophenyl 5-(((((S)-1-oxo-1-propoxypropan-2-yl)amino)(phenoxy)phosphoryl)methyl)benzo[b]thiophene-2-carboxylate

To a solution of 5-(((((S)-1-oxo-1-propoxypropan-2-yl)amino)(phenoxy)phosphoryl)methyl)benzo[b]thiophene-2-carboxylic acid (19 g, 41 mmol, 1 eq) and pyridine (11 g, 0.14 mol, 3.5 eq) in DMF (100 mL) was cooled to 0° C., then the solution of perfluorophenyl 2,2,2-trifluoroacetate (57 g, 0.21 mol, 5 eq) was dropwise added to the mixture at 0° C. Then the mixture was stirred at 0° C. for 1 h to give a yellow solution. The mixture was diluted with water (500 mL) and extracted with EtOAc (500 mL×3), the combined organic layers were dried over Na₂SO₄, filtered and concentrated under reduced pressure to give a residue. The residue was purified by flash silica gel chromatography to give perfluorophenyl 5-(((((S)-1-oxo-1-propoxypropan-2-yl)amino)(phenoxy)phosphoryl)methyl)benzo[b]thiophene-2-carboxylate (17 g, 27 mmol, 65% yield) was obtained as a yellow oil. LCMS (ESI) m/z=628.1 [M+H]⁺. ¹H NMR (400 MHz, CDCl₃) δ 8.33-8.23 (m, 1H), 8.03-7.85 (m, 2H), 7.60-7.53 (m, 1H), 7.28 (s, 2H), 7.20-7.05 (m, 3H), 4.08-3.87 (m, 3H), 3.55-3.42 (m, 2H), 1.63-1.52 (m, 2H), 1.19-1.15 (m, 3H), 0.95-0.83 (m, 3H).

### Synthesis of perfluorophenyl 5-((S)-fluoro((R)-(((S)-1-oxo-1-propoxypropan-2-yl)amino)(phenoxy)phosphoryl)methyl)benzo[b]thiophene-2-carboxylate

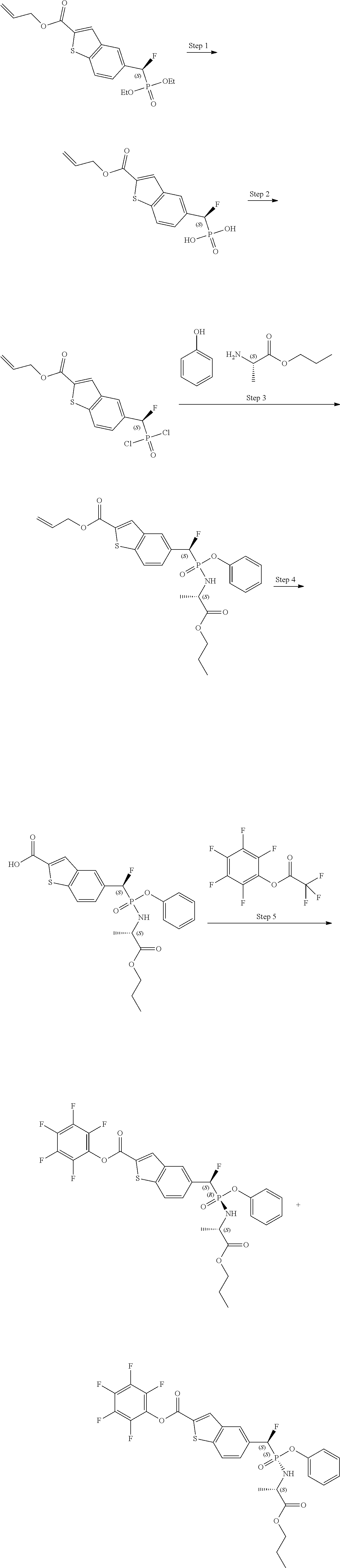

### Step 1: Preparation of (S)-((2-((allyloxy)carbonyl)benzo[b]thiophen-5-yl)fluoromethyl)phosphonic acid

To a solution of allyl (S)-5-((diethoxyphosphoryl)fluoromethyl)benzo[b]thiophene-2-carboxylate (20 g, 52 mmol, 1 eq) in methylene chloride (500 mL) was added trimethylsilyl iodide (21 g, 0.10 mol, 2 eq). The mixture was stirred at 0° C. for 1 hour to give a brown solution. The reaction mixture was concentrated under reduced pressure to give a residue. The reaction residue was purified by prep-HPLC (TFA) to lyophilized to give (S)-((2-((allyloxy)carbonyl)benzo[b]thiophen-5-yl)fluoromethyl)phosphonic acid (12 g, 36 mmol, 70% yield) as a brown solid. LCMS (ESI) m/z=330.9

### Step 2: Preparation of allyl (S)-5-((dichlorophosphoryl)fluoromethyl)benzo[b]thiophene-2-carboxylate

To a solution of (S)-((2-((allyloxy)carbonyl)benzo[b]thiophen-5-yl)fluoromethyl)phosphonic acid (11 g, 33 mmol, 1 eq) in methylene chloride (200 mL) was added dimethylformamide (0.24 g, 3.3 mmol, 0.1 eq) at 0° C. under N₂ atmosphere, then oxalyl chloride (13 g, 0.10 mol, 3 eq) was added dropwise and stirred at 0° C. for 30 minutes and warmed to 40° C. for 1 hour to give a brown solution. The reaction mixture was concentrated under reduced pressure to give allyl (S)-5-((dichlorophosphoryl)fluoromethyl)benzo[b]thiophene-2-carboxylate (11 g crude) as a yellow solid. LCMS (ESI) m/z=358.9.

### Step 3: Preparation of allyl 5-((1S)-fluoro((((S)-1-oxo-1-propoxypropan-2-yl)amino)(phenoxy)phosphoryl)methyl)benzo[b]thiophene-2-carboxylate

To a solution of (S)-5-((dichlorophosphoryl)fluoromethyl)benzo[b]thiophene-2-carboxylate (11 g, 30 mmol, 1 eq) in methylene chloride (150 mL) was added phenol (2.2 g, 24 mmol, 0.8 eq) in methylene chloride (20 mL) in 10 minutes at 0° C., then the solution of N,N-diisopropylethylamine (12 g, 90 mmol, 3 eq) in methylene chloride (200 mL) was dropwise over 1.5 hours and the mixture was stirred at 25° C. for 5 minutes to give a yellow clean solution then the solution of propyl (2S)-2-aminopropanoate (3.9 g, 30 mmol, 1 eq) in methylene chloride (20 mL) was added, the mixture was stirred at 25° C. for 1 hour to give a yellow clean solution. The reaction mixture was concentrated under reduced pressure to give a residue. The residue was purified by flash silica gel chromatography to give allyl 5-((1S)-fluoro((((S)-1-oxo-1-propoxypropan-2-yl)amino)(phenoxy)phosphoryl)methyl)benzo[b]thiophene-2-carboxylate (7.4 g, 14.2 mmol, 47% yield) as a yellow solid. LCMS (ESI) m/z=520.2

### Step 4: Preparation of 5-((1S)-fluoro((((S)-1-oxo-1-propoxypropan-2-yl)amino)(phenoxy)phosphoryl)methyl)benzo[b]thiophene-2-carboxylic acid

To a solution of allyl 5-((1S)-fluoro((((S)-1-oxo-1-propoxypropan-2-yl)amino)(phenoxy)phosphoryl)methyl)benzo[b]thiophene-2-carboxylate (3.5 g, 6.7 mmol, 1 eq) in methylene chloride (40 mL) at 0° C. under N₂ atmosphere, then pyrrolidine (0.38 g, 5.4 mmol, 0.8 eq) and Pd(PPh₃)₄ (0.7 g, 0.67 mmol, 0.11 eq) was added dropwise and stirred at 0° C. for 10 minutes and warmed to 25° C. for 10 minutes to give a brown solution. The reaction mixture was concentrated under reduced pressure to give a residue. The reaction residue was purified by prep-HPLC (TFA) to lyophilized to give 5-((1S)-fluoro((((S)-1-oxo-1-propoxypropan-2-yl)amino)(phenoxy)phosphoryl)methyl)benzo[b]thiophene-2-carboxylic acid (2.8 g, 5.9 mmol, 87% yield) as a white solid. ¹H NMR (400 MHz, CDCl₃) δ 8.10 (d, J=11.2 Hz, 2H), 7.97 (d, J=8.4 Hz, 1H), 7.66 (d, J=8.4 Hz, 1H), 7.38-7.30 (m, 2H), 7.19 (d, J=7.6 Hz, 3H), 6.20-5.99 (m, 1H), 4.05-3.77 (m, 3H), 1.68-1.42 (m, 2H), 1.22 (d, J=7.2 Hz, 3H), 0.89-0.85 (m, 3H)

### Step 5: Preparation of perfluorophenyl 5-((S)-fluoro((R)-(((S)-1-oxo-1-propoxypropan-2-yl)amino)(phenoxy)phosphoryl)methyl)benzo[b]thiophene-2-carboxylate and perfluorophenyl 5-((S)-fluoro((S)-(((S)-1-oxo-1-propoxypropan-2-yl)amino)(phenoxy)phosphoryl)methyl)benzo[b]thiophene-2-carboxylate

To a solution of 5-((1S)-fluoro((((S)-1-oxo-1-propoxypropan-2-yl)amino)(phenoxy)phosphoryl)methyl)benzo[b]thiophene-2-carboxylic acid (3.0 g, 6.3 mmol, 1 eq) in pyridine (15 mL) at 0° C. under N₂ atmosphere, then 2,3,4,5,6-pentafluorophenyl 2,2,2-trifluoroacetate (5.2 g, 19 mmol, 3 eq) was added dropwise and stirred at 0° C. for 10 minutes and warmed to 25° C. for 1 hour to give a brown solution. The reaction mixture was concentrated under reduced pressure to give a residue. The residue was purified by flash silica gel chromatography in 3:1 Petroleum ether/ethyl acetate to give the separated Phos isomers. The analogous (R)—F isomer was made in a similar manner. Analytical data for all 4 isomers are listed in the table below. SFC conditions used in the peak assignments are as follows: Chiralpak AS-3 50×4.6 mm I.D., 3 um. Mobile phase: Phase A for CO₂, and Phase B for EtOH (0.05% DEA); Gradient elution: B in A from 5% to 40%. Flow rate: 3 mL/min; Detector: PDA; Column Temp: 35C; Back Pressure: 100 Bar.

| Name | Structure | LCMS | NMR |
|---|---|---|---|
| perfluorophenyl 5- ((S)-fluoro((R)-(((S)- 1-oxo-1- propoxypropan-2- yl)amino)(phenoxy)phos- phoryl)methyl)benzo [b]thiophene-2- carboxylate | 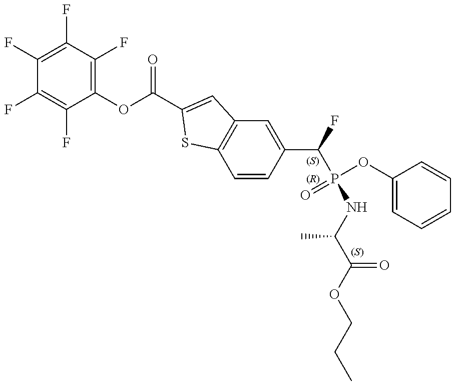 | 645.9 [M + H]⁺ | ¹H NMR (400 MHz, CD₃OD) δ 8.52 (s, 1H), 8.25 (s, 1H), 8.14 (d, J = 8.4 Hz, 1H), 7.81 (d, J = 8.4 Hz, 1H), 7.39-7.32 (m, 2H), 7.27-7.16 (m, 3H), 6.25-6.01 (m, 1H), 4.00 (t, J = 6.8 Hz, 2H), 3.92-3.82 (m, 1H), 1.63-1.52 (m, 2H), 1.14 (d, J = 7.2 Hz, 3H), 0.90 (t, J = 7.2 Hz, 3H) |
| or | or | | |
| perfluorophenyl 5- ((S)-fluoro((S)-(((S)- 1-oxo-1- propoxypropan-2- yl)amino)(phenoxy)phos- phoryl)methyl)benzo [b]thiophene-2- carboxylate | 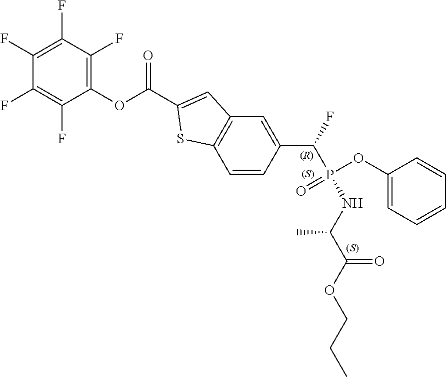 | | |
| | CFTE-27B, SFC Peak 2 | | |
| | Ret. Time = 1.46 Minutes | | |
| perfluorophenyl 5- ((S)-fluoro((R)-(((S)- 1-oxo-1- propoxypropan-2- yl)amino)(phenoxy)phos- phoryl)methyl)benzo [b]thiophene-2- carboxylate | 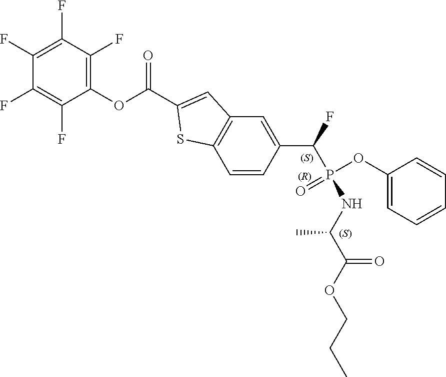 | 646.0 [M + H]⁺ | ¹H NMR (400 MHz, CD₃OD) δ 8.51 (s, 1H), 8.27 (s, 1H), 8.11 (d, J = 8.4 Hz, 1H), 7.79 (d, J = 8.8 Hz, 1H), 7.40-7.33 (m, 2H), 7.25-7.18 (m, 3H), 6.27-6.08 (m, 1H), 4.03-3.85 (m, 3H), 1.63-1.51 (m, 2H), 1.25 (d, J = 7.2 Hz, 3H), 0.89 (t, J = 7.6 Hz, 3H) |
| or | or | | |
| perfluorophenyl 5- ((S)-fluoro((S)-(((S)- 1-oxo-1- propoxypropan-2- yl)amino)(phenoxy)phos- phoryl)methyl)benzo [b]thiophene-2- carboxylate | 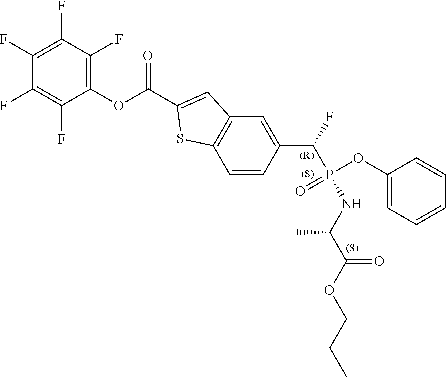 | | |
| | CFTE-27A, SFC Peak 1 | | |
| | Ret. Time = 0.89 Minutes | | |
| perfluorophenyl 5- ((R)-fluoro((R)-(((S)- 1-oxo-1- propoxypropan-2- yl)amino)(phenoxy)phos- phoryl)methyl)benzo [b]thiophene-2- carboxylate | 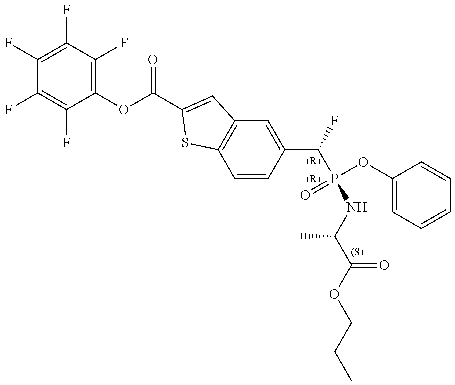 | 645.9 [M + H]⁺ | ¹H NMR (400 MHz, CD₃OD) δ 8.50-8.44 (m, 1H), 8.26-8.18 (m, 1H), 8.13-8.03 (m, 1H), 7.81-7.72 (m, 1H), 7.41-7.28 (m, 2H), 7.27-7.11 (m, 3H), 6.22-6.00 (m, 1H), 4.01-3.94 (m, 2H), 3.93-3.83 (m, 1H), 1.63-1.51 (m, 2H), 1.23 (d, J = 7.2 Hz, 3H), 0.93- 0.83 (m, 3H) |
| or | or | | |
| perfluorophenyl 5- ((R)-fluoro((S)-(((S)- 1-oxo-1- propoxypropan-2- yl)amino)(phenoxy)phos- phoryl)methyl)benzo [b]thiophene-2- carboxylate | 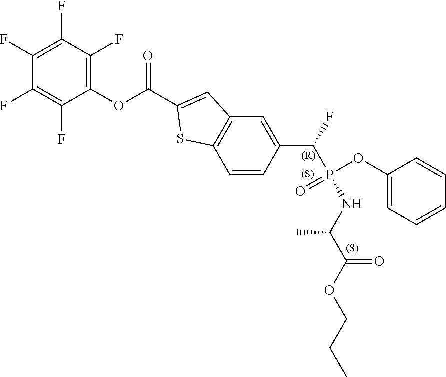 | | |
| | CFTE-28A, SFC Peak 2 | | |
| | Ret. Time = 1.95 Minutes | | |
| perfluorophenyl 5- ((R)-fluoro((R)-(((S)- 1-oxo-1- propoxypropan-2- yl)amino)(phenoxy)phos- phoryl)methyl)benzo [b]thiophene-2- carboxylate | 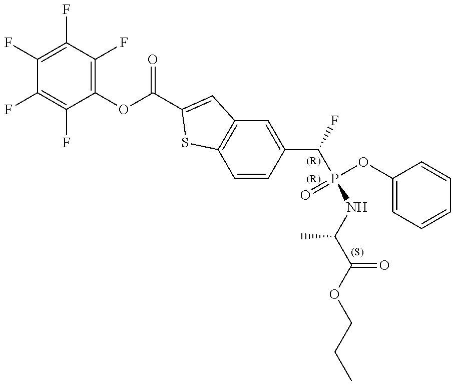 | 646.1 [M + H]⁺ | ¹H NMR (400 MHz, METHANOL-d4) δ 8.40 (s, 1H), 8.17- 8.06 (m, 1H), 8.04- 7.95 (m, 1H), 7.75- 7.64 (m, 1H), 7.29- 7.18 (m, 2H), 7.14- 6.98 (m, 3H), 6.18- 5.93 (m, 1H), 3.87- 3.79 (m, 1H), 3.79- 3.67 (m, 2H), 1.48- 1.36 (m, 2H), 1.15- 1.09 (m, 3H), 0.76 (t, J = 7.6 Hz, 3H) |
| or | or | | |
| perfluorophenyl 5- ((R)-fluoro((S)-(((S)- 1-oxo-1- propoxypropan-2- yl)amino)(phenoxy)phos- phoryl)methyl)benzo [b]thiophene-2- carboxylate | 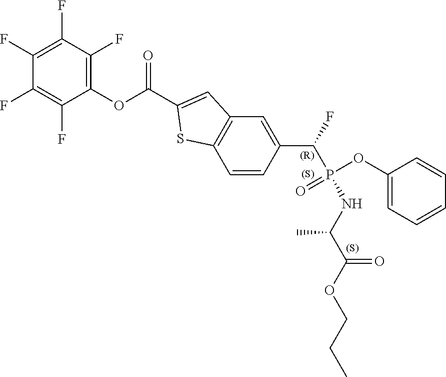 | | |
| | CFTE-28B, SFC Peak 1 | | |
| | Ret. Time = 1.51 Minutes | | |

The intermediates in the table below were prepared using the synthetic procedure described above Synthesis of perfluorophenyl 5-((S)-fluoro((R)-(((S)-1-oxo-1-propoxypropan-2-yl)amino)(phenoxy)phosphoryl)methyl)benzo[b]thiophene-2-carboxylate and utilizing the appropriate starting materials and modifications.

| Name | Structure | LCMS | NMR |
|---|---|---|---|
| perfluorophenyl 5- ((S)-fluoro((R)-(((S)- 1-isopropoxy-1- oxopropan-2- yl)amino)(phenoxy)phos- phoryl)methyl)benzo [b]thiophene-2- carboxylate | 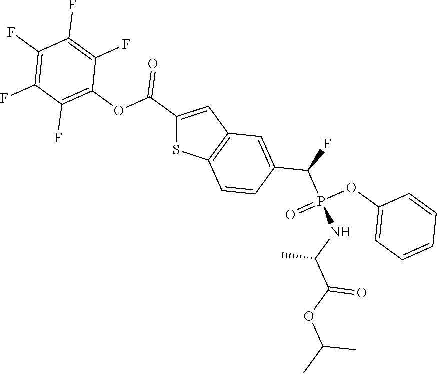 [SMILES: CC(C)OC(=O)[C@H](C)N[P@](=O)(Oc1ccccc1)[C@H](F)c1ccc2sc(C(=O)Oc3c(F)c(F)c(F)c(F)c3F)cc2c1] | 646.2 [M + H]⁺ | |
| or | or | | |
| perfluorophenyl 5- ((S)-fluoro((S)-(((S)-1- isopropoxy-1- oxopropan-2- yl)amino)(phenoxy)phos- phoryl)methyl)benzo [b]thiophene-2- carboxylate | 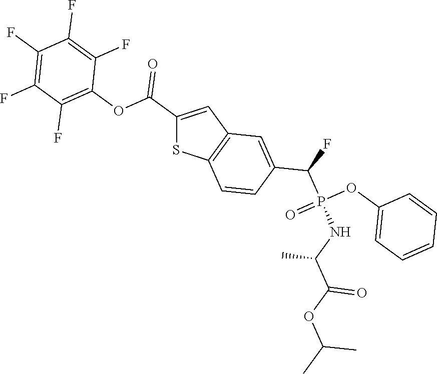 [SMILES: CC(C)OC(=O)[C@H](C)N[P@@](=O)(Oc1ccccc1)[C@H](F)c1ccc2sc(C(=O)Oc3c(F)c(F)c(F)c(F)c3F)cc2c1] | | |
| perfluorophenyl 5- ((S)-fluoro((R)-(((S)- 1-isopropoxy-1- oxopropan-2- yl)amino)(phenoxy)phos- phoryl)methyl)benzo [b]thiophene-2- carboxylate | 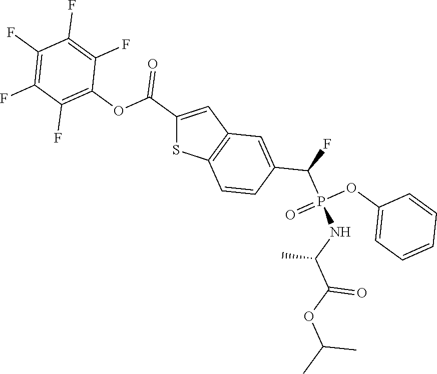 [SMILES: CC(C)OC(=O)[C@H](C)N[P@](=O)(Oc1ccccc1)[C@H](F)c1ccc2sc(C(=O)Oc3c(F)c(F)c(F)c(F)c3F)cc2c1] | 646.2 [M + H]⁺ | ¹H NMR (400 MHz, CDCl₃) δ 8.36 (s, 1H), 8.14 (s, 1H), 8.02-7.95 (m, 1H), 7.76 (d, J = 8.4 Hz, 1H), 7.32-7.28 (m, 2H), 7.18-7.14 (m, 1H), 7.12 (d, J = 8.0 Hz, 2H), 6.10-5.94 (m, 1H), 5.05 (m, 1H), 4.20-4.09 (m, 1H), 3.68 (t, J = 10.4 Hz, 1H), 1.32 (d, J = 7.2 Hz, 3H), 1.30-1.24 (m, 6H) |
| or | or | | |
| perfluorophenyl 5- ((S)-fluoro((S)-(((S)-1- isopropoxy-1- oxopropan-2- yl)amino)(phenoxy)phos- phoryl)methyl)benzo [b]thiophene-2- carboxylate | 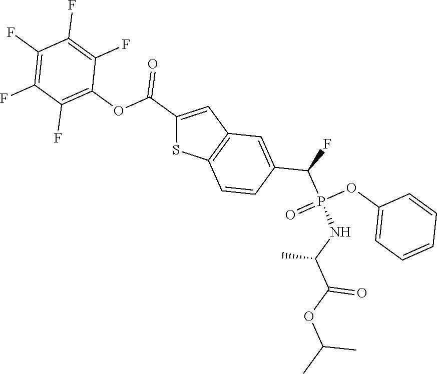 [SMILES: CC(C)OC(=O)[C@H](C)N[P@@](=O)(Oc1ccccc1)[C@H](F)c1ccc2sc(C(=O)Oc3c(F)c(F)c(F)c(F)c3F)cc2c1] | | |

The intermediates in the table below were prepared using the synthetic procedure described above for allyl 5-((1S)-fluoro((((S)-1-oxo-1-propoxypropan-2-yl)amino)(phenoxy)phosphoryl)methyl)benzo[b]thiophene-2-carboxylate and utilizing the appropriate starting materials and modifications.

| Name | Structure | LCMS |
|---|---|---|
| allyl 5-((1R)-fluoro((((S)-1- oxo-1-propoxypropan-2- yl)amino)(phenoxy)phosphoryl) methyl)benzo[b]thiophene-2- carboxylate | 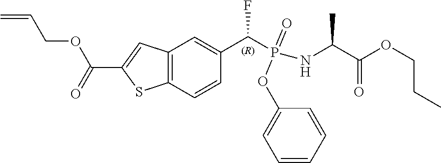 | 520.1 [M + H]⁺ |

The chiral intermediates in the table below were obtained by chiral SFC separation of the corresponding P-isomer mixtures, which were prepared using the synthetic procedure described above for allyl 5-((S) fluoro((((S)-h-oxo-1 propoxypropan-2-yl)amino)(phenoxy)phosphoryl)methyl)benzo[b]thiophene-2-carboxylate and utilizing the appropriate starting materials and modifications. Absolute stereochemical configuration at phosphorus is arbitrarily assigned as drawn.

| | | | Preparative SFC |
|---|---|---|---|
| | | | conditions for |
| Name | Structure | LCMS | separation |
| allyl 5-((S)-fluoro((R)- (((S)-1-oxo-1- propoxypropan-2- yl)amino)(phenoxy)phos- phoryl)methyl)benzo [b]thiophene-2- carboxylate | 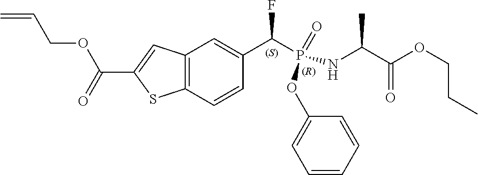 | 520.2 [M + H]⁺ | ChiralPak R,R- WHELK 250 × 21.2 mm 5 μm column; Mobile phase: A for CO₂ and B for MeOH + 0.1% NH₃H₂O; Gradient: B 50%; Flow rate: 40 mL/min; Back pressure: 100 bar; Column temperature: 35° C.; Wavelength: |
| | (Peak 1, RT = 4.46 min) | | 220 nm; Cycle-time: |
| allyl 5-((S)-fluoro((S)- (((S)-1-oxo-1- propoxypropan-2- yl)amino)(phenoxy)phos- phoryl)methyl)benzo [b]thiophene-2- carboxylate | 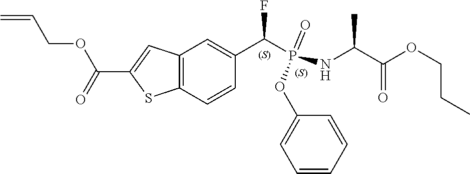 | 520.2 [M + H]⁺ | 11 min |
| | (Peak 2, RT = 6.54 min) | | |
| allyl 5-((R)- fluoro((R)-(((S)-1- oxo-1-propoxypropan- 2- yl)amino)(phenoxy)phos- phoryl)methyl)benzo [b]thiophene-2- carboxylate | 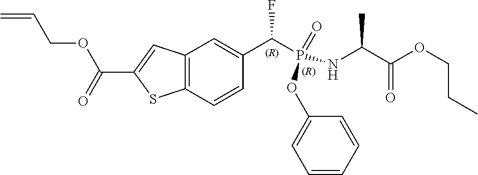 | 520.2 [M + H]⁺ | ChiralPak IH 250 × 21.2 mm 5 μm column; Mobile phase: A for CO₂ and B for MeOH + 0.1% NH₃H₂O; Gradient: B 35%; Flow rate: 40 mL/min; Back pressure: 100 bar; Column temperature: 35° C.; Wavelength: 220 nm; Cycle-time: 5 |
| | (Peak 1, RT = 2.72 min) | | min |
| allyl 5-((R)-fluoro((S)- (((S)-1-oxo-1- propoxypropan-2- yl)amino)(phenoxy)phos- phoryl)methyl)benzo [b]thiophene-2- carboxylate | 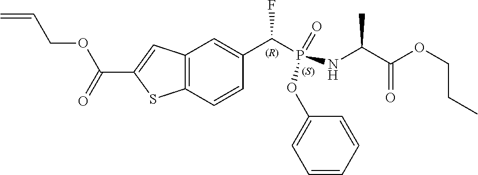 | 520.2 [M + H]⁺ | |
| | (Peak 2, RT = 4.11 min) | | |

The intermediates in the table below were prepared using the synthetic procedure described above for 5-((1S)-fluoro((((S)-1-oxo-1-propoxypropan-2-yl)amino)(phenoxy)phosphoryl)methyl)benzo[b]thiophene-2-carboxylic acid and utilizing the appropriate starting materials and modifications.

| Name | Structure | LCMS |
|---|---|---|
| 5-((S)-fluoro((R)-(((S)-1-oxo-1- propoxypropan-2- yl)amino)(phenoxy)phosphoryl) methyl)benzo[b]thiophene-2- carboxylic acid | 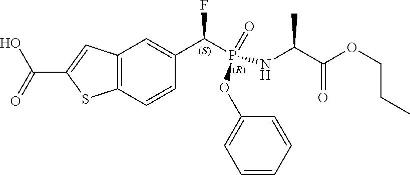 | 480.1 [M + H]⁺ |
| 5-((S)-fluoro((S)-(((S)-1-oxo-1- propoxypropan-2- yl)amino)(phenoxy)phosphoryl) methyl)benzo[b]thiophene-2- carboxylic acid | 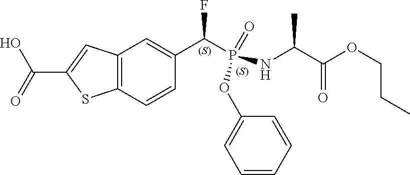 | 480.1 [M + H]⁺ |
| 5-((R)-fluoro((R)-(((S)-1-oxo- 1-propoxypropan-2- yl)amino)(phenoxy)phosphoryl) methyl)benzo[b]thiophene-2- carboxylic acid | 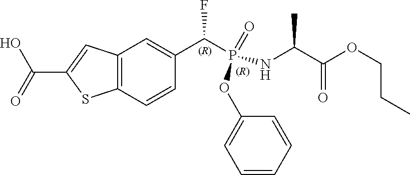 | 480.1 [M + H]⁺ |
| 5-((R)-fluoro((S)-(((S)-1-oxo-1- propoxypropan-2- yl)amino)(phenoxy)phosphoryl) methyl)benzo[b]thiophene-2- carboxylic acid | 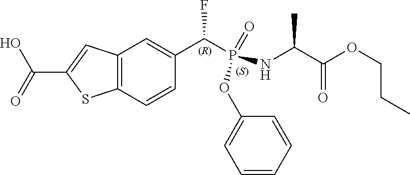 | 480.1 [M + H]⁺ |

The intermediates in the table below were prepared using the protocol described above for synthesis of 5-(((((S)-1-isopropoxy-1-oxopropan-2-yl)amino)(phenoxy)phosphoryl)methyl)benzo[b]thiophene-2-carboxylic acid and utilizing the appropriate starting materials and modifications.

| Name | Structure | LCMS |
|---|---|---|
| 5-(((((S)-1-ethoxy-1-oxopropan-2- yl)amino)(2,2,2- trifluoroethoxy)phosphoryl)fluoromethyl) benzo[b]thiophene-2-carboxylic acid | 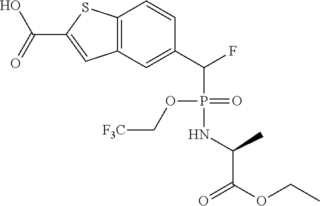 [SMILES: CCOC(=O)[C@H](C)NP(=O)(OCC(F)(F)F)C(F)c1ccc2sc(C(=O)O)cc2c1] | 472.1 [M + H]⁺ |
| 5-(((((S)-1-oxo-1-propoxypropan-2- yl)amino)(2,2,2- trifluoroethoxy)phosphoryl)methyl)benzo [b]thiophene-2-carboxylic acid | 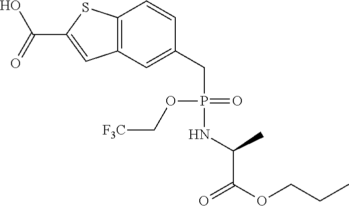 [SMILES: CCCOC(=O)[C@H](C)NP(=O)(Cc1ccc2sc(C(=O)O)cc2c1)OCC(F)(F)F] | 468.0 [M + H]⁺ |
| 5-(fluoro((((S)-1-oxo-1-propoxypropan- 2-yl)amino)(2,2,2- trifluoroethoxy)phosphoryl)methyl)benzo [b]thiophene-2-carboxylic acid | 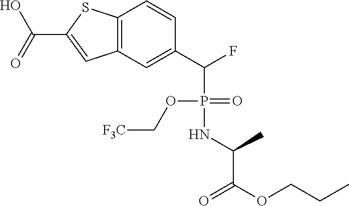 [SMILES: CCCOC(=O)[C@H](C)NP(=O)(OCC(F)(F)F)C(F)c1ccc2sc(C(=O)O)cc2c1] | 485.9 [M + H]⁺ |
| 5-(fluoro((((S)-1-oxo-1-propoxybutan-2- yl)amino)(phenoxy)phosphoryl)methyl)ben- zo[b]thiophene-2-carboxylic acid | 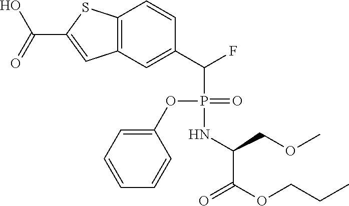 | 494.1 [M + H]⁺ |
| 5-(fluoro((((S)-3-methoxy-1-oxo-1- propoxypropan-2- yl)amino)(phenoxy)phosphoryl)methyl)ben- zo[b]thiophene-2-carboxylic acid | 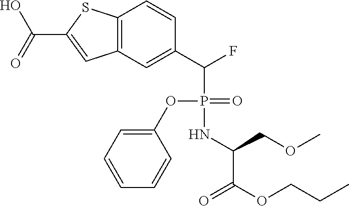 [SMILES: CCCOC(=O)[C@H](COC)NP(=O)(Oc1ccccc1)C(F)c1ccc2sc(C(=O)O)cc2c1] | 510.2 [M + H]⁺ |

### Synthesis of perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate and perfluorophenyl 5-((R)-fluoro((R)-(((S)-1-(neopentyloxy)-1-oxopropan-2-yl)amino)(phenoxy)phosphoryl)methyl)benzo[b]thiophene-2-carboxylate

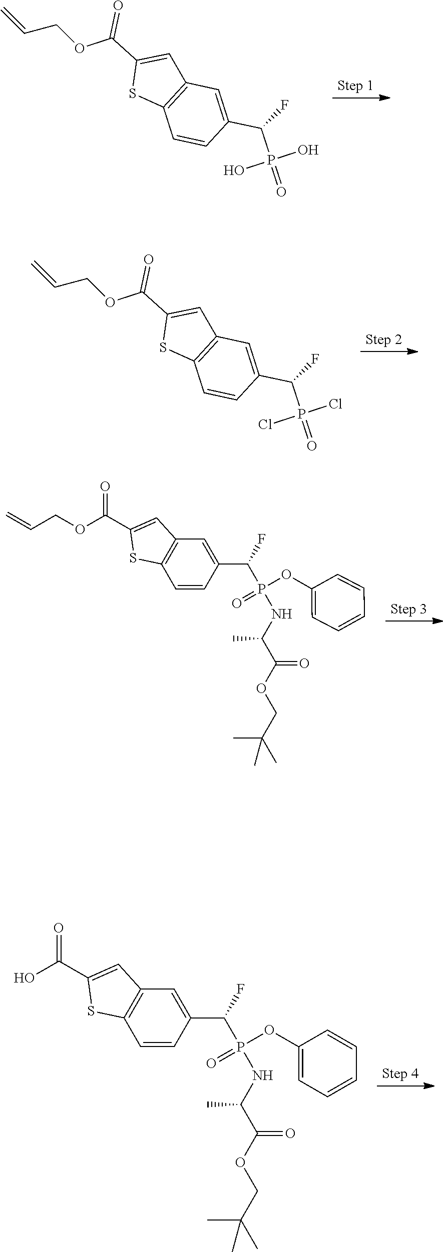 [SMILES: C=CCOC(=O)c1cc2cc([C@H](F)P(=O)(O)O)ccc2s1 ; C=CCOC(=O)c1cc2cc([C@H](F)P(=O)(Cl)Cl)ccc2s1 ; C=CCOC(=O)c1cc2cc([C@H](F)P(=O)(N[C@@H](C)C(=O)OCC(C)(C)C)Oc3ccccc3)ccc2s1 ; C[C@H](NP(=O)(Oc1ccccc1)[C@@H](F)c1ccc2sc(C(=O)O)cc2c1)C(=O)OCC(C)(C)C]

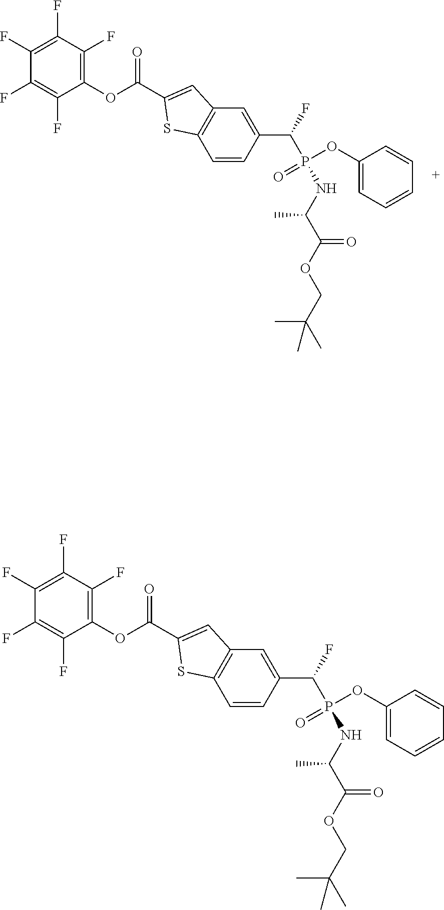 [SMILES: C[C@H](N[P@@](=O)(Oc1ccccc1)[C@@H](F)c1ccc2sc(C(=O)Oc3c(F)c(F)c(F)c(F)c3F)cc2c1)C(=O)OCC(C)(C)C ; C[C@H](N[P@](=O)(Oc1ccccc1)[C@@H](F)c1ccc2sc(C(=O)Oc3c(F)c(F)c(F)c(F)c3F)cc2c1)C(=O)OCC(C)(C)C]

### Step 1: Preparation of allyl (R)-5-((dichlorophosphoryl)fluoromethyl)benzo[b]thiophene-2-carboxylate

To a solution of (R)-((2-((allyloxy)carbonyl)benzo[b]thiophen-5-yl)fluoromethyl)phosphonic acid (2.0 g, 6.1 mmol, 1.0 eq) in DCM (30 mL) was added 3 drops of DMF (0.03 mL) cooled to 0° C., then the reaction was added oxalyl chloride (2.3 g, 18.1 mmol, 3 eq) slowly, after that the reaction was warmed to 45° C. and stirred 20 min to give a yellow clean solution. The reaction mixture was concentrated under reduced pressure to give allyl (R)-5-((dichlorophosphoryl)fluoromethyl)benzo[b]thiophene-2-carboxylate (2.0 g, crude) as a yellow oil and was used into the next Step without further purification. LCMS (ESI) m/z=358.9 [M+H]⁺.

### Step 2: Preparation of allyl 5-((1R)-fluoro((((S)-1-(neopentyloxy)-1-oxopropan-2-yl)amino)(phenoxy)phosphoryl)methyl)benzo[b]thiophene-2-carboxylate

To a solution of allyl (R)-5-((dichlorophosphoryl)fluoromethyl)benzo[b]thiophene-2-carboxylate (2.0 g, 5.4 mmol, 1.0 eq) in DCM (40 mL) was cooled to 0° C. and was added a solution of phenol (0.5 g, 5.4 mmol, 1.0 eq) in DCM (10 mL), then the mixture was added N,N-diisopropylethylamine (2.1 g, 16 mmol, 3.0 eq) in DCM (40 mL) dropwise. The reaction was stirred at 0° C. for 10 min and was added 2,2-dimethylpropyl (2S)-2-aminopropanoate (0.9 g, 5.4 mmol, 1.0 eq) in DCM (20 mL). Then the reaction was warmed to 25° C. and stirred for 1 h to give a light-yellow clean solution. The reaction mixture was concentrated under reduced pressure to give a residue. The residue was purified by flash column chromatography on silica gel to get allyl 5-((1R)-fluoro((((S)-1-(neopentyloxy)-1-oxopropan-2-yl)amino)(phenoxy)phosphoryl)methyl)benzo[b]thiophene-2-carboxylate (1.7 g, 3.1 mmol, 57% yield) was obtained as a yellow oil. LCMS (ESI) m/z=548.2. ¹H NMR (400 MHz, CDCl₃) δ 8.10 (s, 1H), 8.07-7.99 (m, 1H), 7.91 (d, J=8.4 Hz, 1H), 7.68-7.59 (m, 1H), 7.41-7.25 (m, 3H), 7.23-7.06 (m, 3H), 6.12-5.90 (m, 2H), 5.48-5.44 (m, 1H), 5.39-5.30 (m, 1H), 4.87 (d, J=5.6 Hz, 2H), 4.22-4.07 (m, 2H), 3.90-3.83 (m, 1H), 3.74-3.63 (m, 2H), 1.38-1.30 (m, 3H), 0.92 (s, 9H).

### Step 3: Preparation of 5-((1R)-fluoro((((S)-1-(neopentyloxy)-1-oxopropan-2-yl)amino)(phenoxy)phosphoryl)methyl)benzo[b]thiophene-2-carboxylic acid

To a solution of allyl 5-((1R)-fluoro((((S)-1-(neopentyloxy)-1-oxopropan-2-yl)amino)(phenoxy)phosphoryl)methyl)benzo[b]thiophene-2-carboxylate (1.7 g, 3.1 mmol, 1.0 eq) in DCM (20 mL) was added pyrrolidine (0.4 g, 6.2 mmol, 2.0 eq) and Pd(PPh₃)₄ (0.4 g, 0.3 mmol, 0.1 eq). Then the reaction stirred for 15 min to give a yellow clean solution. The reaction mixture was concentrated under reduced pressure to give a residue. The residue was purified by reversed phase (TFA) to lyophilized to give 5-((1R)-fluoro((((S)-1-(neopentyloxy)-1-oxopropan-2-yl)amino)(phenoxy)phosphoryl)methyl)benzo[b]thiophene-2-carboxylic acid (1.3 g, 2.6 mmol, 83% yield) as a white solid. LCMS (ESI) m/z=508.2

### Step 4: Preparation of perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate and perfluorophenyl 5-((R)-fluoro((R)-(((S)-1-(neopentyloxy)-1-oxopropan-2-yl)amino)(phenoxy)phosphoryl)methyl)benzo[b]thiophene-2-carboxylate

To a solution of 5-((1R)-fluoro((((S)-1-(neopentyloxy)-1-oxopropan-2-yl)amino)(phenoxy)phosphoryl)methyl)benzo[b]thiophene-2-carboxylic acid (1.2 g, 2.4 mmol, 1.0 eq) in pyridine (100 mL) was added 2,3,4,5,6-pentafluorophenyl 2,2,2-trifluoroacetate (0.66 g, 2.4 mmol, 1.0 eq) at 0° C., the mixture was stirred at 25° C. for 10 min to give a yellow clean solution. The reaction mixture was concentrated under reduced pressure to give a residue. The residue was purified by flash column chromatography on silica gel to give perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate or perfluorophenyl 5-((R)-fluoro((R)-(((S)-1-(neopentyloxy)-1-oxopropan-2-yl)amino)(phenoxy)phosphoryl)methyl)benzo[b]thiophene-2-carboxylate (0.4 g, 0.59 mmol, 25% yield, Rt=1.883 min, Peak 2) as a yellow oil and perfluorophenyl 5-((R)-fluoro((R)-(((S)-1-(neopentyloxy)-1-oxopropan-2-yl)amino)(phenoxy)phosphoryl)methyl)benzo[b]thiophene-2-carboxylate or perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate (0.50 g, 0.74 mmol, 32% yield, Rt=1.442 min, Peak 1) as a yellow oil.

Peak 2: perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate: LCMS (ESI) m/z=674.0 [M+H]⁺. ¹H NMR (400 MHz, CDCl₃) δ 8.34 (s, 1H), 8.09 (s, 1H), 7.97 (d, J=8.4 Hz, 1H), 7.68 (d, J=8.4 Hz, 1H), 7.40-7.35 (m, 2H), 7.32-7.27 (m, 2H), 7.24-7.19 (m, 1H), 6.07-5.89 (m, 1H), 4.16-4.05 (m, 1H), 3.88-3.76 (m, 2H), 3.72-3.64 (m, 1H), 1.15 (d, J=7.2 Hz, 3H), 0.91 (s, 9H)

Peak 1: perfluorophenyl 5-((R)-fluoro((R)-(((S)-1-(neopentyloxy)-1-oxopropan-2-yl)amino)(phenoxy)phosphoryl)methyl)benzo[b]thiophene-2-carboxylate or perfluorophenyl 5-((R)-fluoro((S)-(((S)-1-(neopentyloxy)-1-oxopropan-2-yl)amino)(phenoxy)phosphoryl)methyl)benzo[b]thiophene-2-carboxylate: LCMS (ESI) m/z=674.0 [M+H]⁺. ¹H NMR (400 MHz, CDCl₃) δ 8.34 (s, 1H), 8.12 (s, 1H), 7.96 (d, J=8.4 Hz, 1H), 7.74 (d, J=8.4 Hz, 1H), 7.29 (s, 1H), 7.25 (s, 1H), 7.16-7.05 (m, 3H), 6.13-5.96 (m, 1H), 4.22-4.10 (m, 1H), 3.89-3.83 (m, 1H), 3.73-3.66 (m, 1H), 3.62-3.54 (m, 1H), 1.33 (d, J=7.2 Hz, 3H), 0.92 (s, 9H).

### Synthesis of 5-(difluoro((((S)-1-isopropoxy-1-oxopropan-2-yl)amino)(phenoxy)phosphoryl)methyl)benzo[b]thiophene-2-carboxylic acid

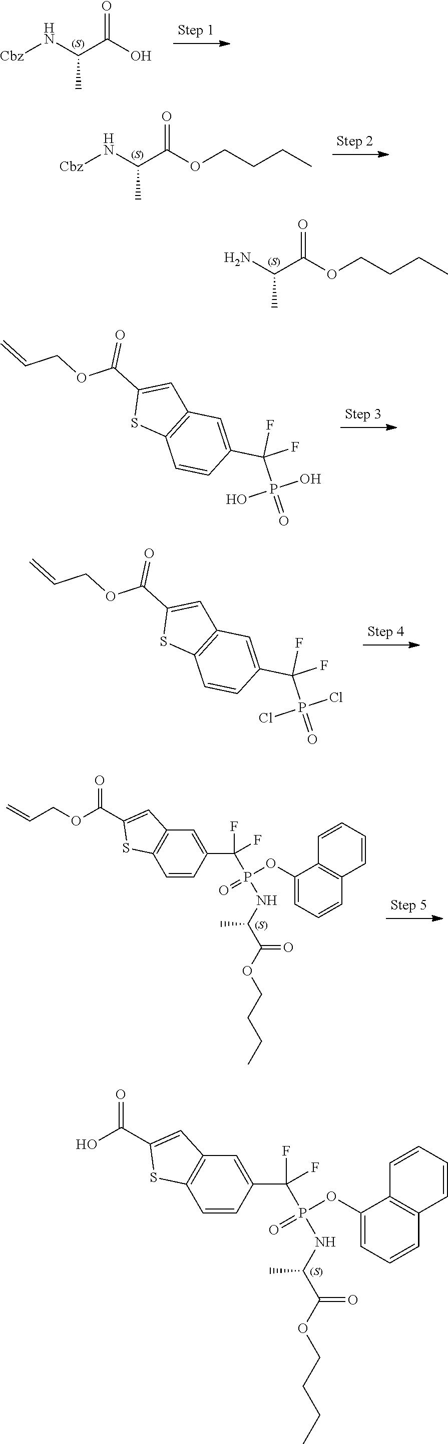

### Step 1: Preparation of allyl 5-((diethoxyphosphoryl)difluoromethyl)benzo[b]thiophene-2-carboxylate

To a solution of 5-((diethoxyphosphoryl)difluoromethyl)benzo[b]thiophene-2-carboxylic acid (5 g, 14 mmol, 1 eq) in dimethyl sulfoxide (40 mL) was added disodium carbonate (4.3 g, 41 mmol, 3 eq) and 3-bromoprop-1-ene (1.7 g, 14 mmol, 1 eq), the mixture was stirred at 25° C. for 12 h to give a white suspension. The mixture was diluted with water (200 mL) and extracted with EtOAc (200 mL×2), the combined organic layers were washed with saturated brine (200 mL×2), dried over Na₂SO₄, filtered and concentrated under reduced pressure to give a residue. The residue was purified by column chromatography to give allyl 5-((diethoxyphosphoryl)difluoromethyl)benzo[b]thiophene-2-carboxylate (4.6 g, 11.3 mmol, 83% yield) as a white oil. ¹H NMR (400 MHz, CDCl₃) δ 8.17-8.13 (m, 2H), 7.96 (d, J=8.8 Hz, 1H), 7.70 (d, J=8.4 Hz, 1H), 6.13-5.99 (m, 1H), 5.50-5.42 (m, 1H), 5.37-5.32 (m, 1H), 4.87 (d, J=5.6 Hz, 2H), 4.27-4.12 (m, 4H), 1.33 (t, J=7.2 Hz, 6H).

### Step 2: Preparation of ((2-((allyloxy)carbonyl)benzo[b]thiophen-5-yl)difluoromethyl)phosphonic acid

To a solution of allyl 5-((diethoxyphosphoryl)difluoromethyl)benzo[b]thiophene-2-carboxylate (4.6 g, 11.3 mmol, 1 eq) in DCM (40 mL) was added iodotrimethylsilane (9.0 g, 45 mmol, 4 eq) at 0° C., the mixture was stirred at 0° C. for 2 h. The reaction mixture was concentrated under reduced pressure to give a residue. The residue was purified by reversed phase (TFA) to give ((2-((allyloxy) carbonyl)benzo[b]thiophen-5-yl)difluoromethyl)phosphonic acid (2.3 g, 6.6 mmol, 58% yield) as a white solid. LCMS (ESI) m/z=348.7

### Step 3: Preparation of allyl 5-((dichlorophosphoryl)difluoromethyl)benzo[b]thiophene-2-carboxylate

To a solution of ((2-((allyloxy) carbonyl)benzo[b]thiophen-5-yl)difluoromethyl)phosphonic acid (1 g, 2.9 mmol, 1 eq) in DCM (3 mL) was added DMF (21 mg, 0.29 mmol, 0.1 eq), oxalic dichloride (1.2 g, 28 mmol, 3 eq) was dropwise at 0° C., the mixture was stirred at 40° C. for 1 h to give a yellow solution. The reaction mixture was concentrated under reduced pressure to give allyl 5-((dichlorophosphoryl)difluoromethyl)benzo[b]thiophene-2-carboxylate (1 g, crude) as a yellow solid. LCMS (ESI) m/z=376.6.

### Step 4: Preparation of allyl 5-(((((S)-1-butoxy-1-oxopropan-2-yl)amino)(naphthalen-1-yloxy)phosphoryl)difluoromethyl)benzo[b]thiophene-2-carboxylate

To a solution of prop-2-en-1-yl 5-[(dichlorophosphoryl)difluoromethyl]-1-benzothiophene-2-carboxylate (0.2 g, 0.52 mmol, 1 eq) in methylene chloride (10 mL) was added naphthalen-1-ol (60 mg, 0.42 mmol, 0.8 eq), the mixture was stirred at 0° C. for 5 min, then a solution of N,N-diisopropylethylamine (0.2 g, 1.6 mmol, 3.0 eq) in methylene chloride (10 mL) was added to the mixture, then a solution of butyl (2S)-2-aminopropanoate (75 mg, 0.52 mmol, 1 eq) in methylene chloride (10 mL) was added to the mixture, the mixture was stirred at 0° C. for 30 min to give yellow solution. The reaction mixture was concentrated under reduced pressure to give a residue.

The residue was purified by column chromatography to give prop-2-en-1-yl 5-[({[(2S)-1-butoxy-1-oxopropan-2-yl]amino}(naphthalen-1-yloxy)phosphoryl)difluoromethyl]-1-benzothiophene-2-carboxylate (0.15 g, 0.24 mmol, 46.4% yield) as a yellow oil. LCMS (ESI) m/z=602.1

### Step 5: Preparation of 5-(((((S)-1-butoxy-1-oxopropan-2-yl)amino)(naphthalen-1-yloxy)phosphoryl)difluoromethyl)benzo[b]thiophene-2-carboxylic acid

To a solution of prop-2-en-1-yl 5-[({[(2S)-1-butoxy-1-oxopropan-2-yl]amino}(naphthalen-1-yloxy)phosphoryl)difluoromethyl]-1-benzothiophene-2-carboxylate (0.14 g, 0.23 mmol, 1 eq) in methylene chloride (1.0 mL) was added Pd(PPh₃)₄ (27 mg, 23 μmol, 0.1 eq), the mixture was stirred at 0° C. for 5 min, then pyrrolidine (17 mg, 0.23 mmol, 1 eq) was added to the mixture, the mixture was stirred at 25° C. for 5 min to give yellow solution. The reaction mixture was concentrated under reduced pressure to give a residue. The residue was purified by reversed phase (TFA) to give 5-[({[(2S)-1-butoxy-1-oxopropan-2-yl]amino}(naphthalen-1-yloxy)phosphoryl)difluoromethyl]-1-benzothiophene-2-carboxylic acid (97 mg, 0.17 mmol, 74.6% yield) as a yellow oil. LCMS (ESI) m/z=562.1.

### Synthesis of 5-(((((S)-1-butoxy-1-oxopropan-2-yl)(methyl)amino)(phenoxy)phosphoryl)methyl)benzo[b]thiophene-2-carboxylic acid

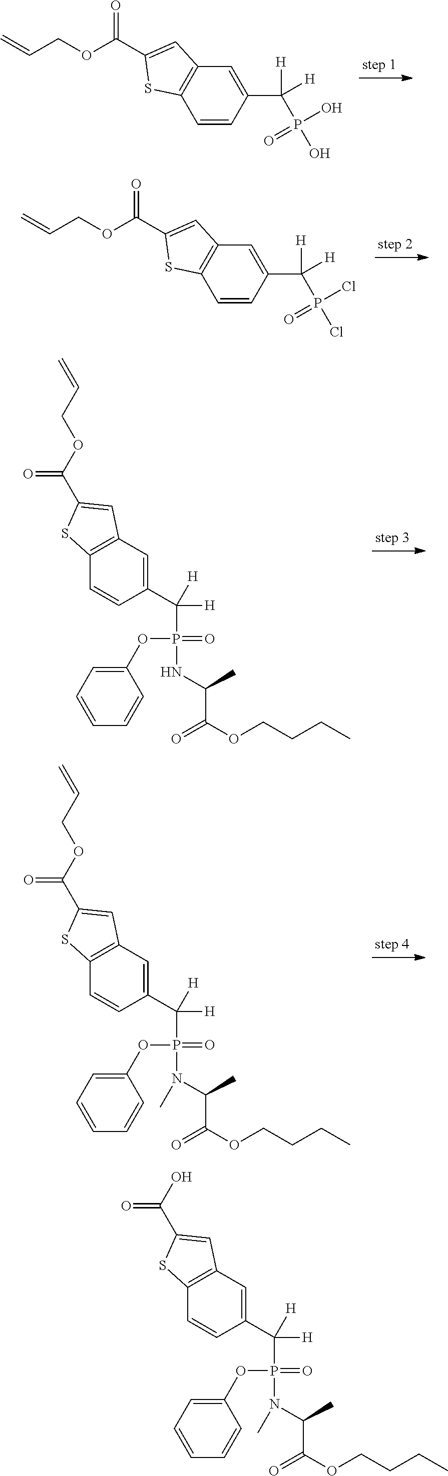

### Step 1: Preparation of allyl 5-((dichlorophosphoryl)methyl)benzo[b]thiophene-2-carboxylate

To a solution of ((2-((allyloxy)carbonyl)benzo[b]thiophen-5-yl)methyl)phosphonic acid (0.1 g, 0.32 mmol, 1 eq) in DCM (2 mL) was added DMF (2.3 mg, 32 μmol, 0.1 eq). The mixture was stirred at 0° C. for 5 min, then a solution of oxalyl chloride (0.12 g, 0.96 mmol, 3 eq) in DCM (2 mL) was added to the mixture, the mixture was stirred at 40° C. for 1 h to give yellow solution. The reaction mixture was concentrated under reduced pressure to give allyl 5-((dichlorophosphoryl)methyl)benzo[b]thiophene-2-carboxylate (0.100 g, crude) as a yellow oil. LCMS (ESI) m/z=341.0 (MeOH quench)

### Step 2: Preparation of allyl 5-(((((S)-1-butoxy-1-oxopropan-2-yl)amino)(phenoxy)phosphoryl)methyl)benzo[b]thiophene-2-carboxylate

To a solution of allyl 5-((dichlorophosphoryl)methyl)benzo[b]thiophene-2-carboxylate (0.10 g, 0.29 mmol, 1 eq) in DCM (2 mL) was added phenol (22 mg, 0.23 mmol, 0.8 eq) at 0° C. for 30 min, then a solution of DIEA (0.1 g, 0.86 mmol, 3.0 eq) in DCM (2 mL) was added to the mixture at 0° C. for 15 min, then a solution of butyl L-alaninate (42 mg, 0.29 mmol, 1 eq) in DCM (2 mL) was added to the mixture at 0° C. for 15 min to give yellow solution. The reaction mixture was concentrated under reduced pressure to give a residue. The residue was purified by column chromatography to give allyl 5-(((((S)-1-butoxy-1-oxopropan-2-yl)amino)(phenoxy)phosphoryl)methyl)benzo[b]thiophene-2-carboxylate (80 mg, 0.15 mmol, 80% yield,) as a yellow oil. LCMS (ESI) m/z=516.2 [M+H]⁺.

### Step 3: Preparation of allyl 5-(((((S)-1-butoxy-1-oxopropan-2-yl)(methyl)amino)(phenoxy)phosphoryl)methyl)benzo[b]thiophene-2-carboxylate

To a solution of allyl 5-(((((S)-1-butoxy-1-oxopropan-2-yl)amino)(phenoxy)phosphoryl)methyl)benzo[b]thiophene-2-carboxylate (0.2 g, 0.39 mmol, 1 eq) in THF (5 mL) was added LDA (83 mg, 0.77 mmol, 2 eq) and methyl iodide (0.55 g, 3.9 mmol, 10 eq), the mixture was stirred at −70° C. for 15 min to give yellow solution. The reaction mixture was added water (0.5 mL) and concentrated under reduced pressure to give a residue. The residue was purified by column chromatography to give allyl 5-(((((S)-1-butoxy-1-oxopropan-2-yl)(methyl)amino)(phenoxy)phosphoryl)methyl)benzo[b]thiophene-2-carboxylate (60 mg, 0.11 mmol, 28% yield) as a yellow oil. LCMS (ESI) m/z=530.2 [M+H]⁺.

### Step 4: Preparation of 5-(((((S)-1-butoxy-1-oxopropan-2-yl)(methyl)amino)(phenoxy)phosphoryl)methyl)benzo[b]thiophene-2-carboxylic acid

To a solution of allyl 5-(((((S)-1-butoxy-1-oxopropan-2-yl)(methyl)amino)(phenoxy)phosphoryl)methyl)benzo[b]thiophene-2-carboxylate (60 mg, 0.11 mmol, 1 eq) in DCM (2 mL) was added pyrrolidine (6.4 mg, 90 μmol, 0.8 eq) and Pd(PPh₃)₄ (1.3 mg, 1.1 μmol, 0.01 eq), the mixture was stirred at 25° C. for 5 min to give yellow solution. The reaction mixture was added water and concentrated under reduced pressure to give a residue. The residue was purified by reversed phase (TFA) then lyophilized to give 5-(((((S)-1-butoxy-1-oxopropan-2-yl)(methyl)amino)(phenoxy)phosphoryl)methyl)benzo[b]thiophene-2-carboxylic acid (49 mg, 99 μmol, 88% yield) as a yellow oil. LCMS (ESI) m/z=490.1 [M+H]⁺.

### Synthesis of perfluorophenyl 5-(fluoro((((S)-1-oxo-1-propoxypropan-2-yl)amino)(phenoxy)phosphoryl)methyl)benzo[b]thiophene-2-carboxylate

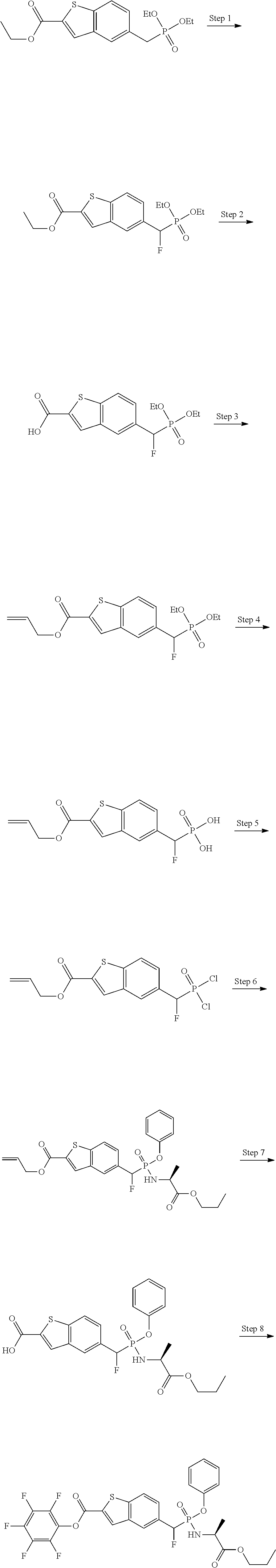

### Step 1: Preparation of ethyl 5-((diethoxyphosphoryl)fluoromethyl)benzo[b]thiophene-2-carboxylate

To a solution of ethyl 5-((diethoxyphosphoryl)methyl)benzo[b]thiophene-2-carboxylate (58 g, 0.16 mol, 1 eq) in THF (600 mL) was added lithium bis(trimethylsilyl)azanide (170 mL, 0.17 mol, 1.1 eq, 1.0 M in THF) at −70° C., then N-(benzenesulfonyl)-N-fluorobenzenesulfonamide (54 g, 0.17 mol, 1.1 eq) was added at −70° C., then stirred at −70° C. for 0.5 h. The reaction mixture was quenched by water (500 mL) at −70° C., then extracted with EtOAc (500 mL×2), the combined organic layers were washed with saturated brine (400 mL×2), dried over Na₂SO₄, filtered and concentrated under reduced pressure to give a residue. The residue was purified by flash silica gel chromatography to give ethyl 5-((diethoxyphosphoryl)fluoromethyl)benzo[b]thiophene-2-carboxylate (26 g, 67 mmol, 43% yield) as a yellow oil. ¹H NMR (400 MHz, CDCl₃) δ 8.00 (s, 1H), 7.92 (s, 1H), 7.83 (d, J=8.4 Hz, 1H), 7.51 (d, J=8.4 Hz, 1H), 5.84-5.63 (m, 1H), 4.39-4.29 (m, 2H), 4.09-3.96 (m, 4H), 1.35 (t, J=7.1 Hz, 3H), 1.23-1.19 (m, 6H).

### Step 2: Preparation of 5-((diethoxyphosphoryl)fluoromethyl)benzo[b]thiophene-2-carboxylic acid

To a solution of ethyl 5-((diethoxyphosphoryl)fluoromethyl)benzo[b]thiophene-2-carboxylate (15 g, 40 mmol, 1 eq) in THF (40 mL) was dropwise added LiOH—H₂O (1.8 g, 44 mmol, 1.1 eq) in H₂O (40 mL) at 0° C. and stirred at 25° C. for 1 h to give a yellow solution. The reaction mixture was extracted with EtOAc (80 mL), then the aqueous phase was used 1M HCl to adjust the pH=4-3 extracted with EtOAc (100 mL×2), the combined organic layers were dried over Na₂SO₄, filtered and concentrated under reduced pressure to give 5-((diethoxyphosphoryl)fluoromethyl)benzo[b]thiophene-2-carboxylic acid (11 g, crude) as a white solid. LCMS (ESI) m/z=346.8 [M+H]⁺.

### Step 3: Preparation of allyl 5-((diethoxyphosphoryl)fluoromethyl)benzo[b]thiophene-2-carboxylate

To a solution of 5-((diethoxyphosphoryl)fluoromethyl)benzo[b]thiophene-2-carboxylic acid (11 g, 32 mmol, 1 eq) in DMF (120 mL) was added sodium carbonate (6.7 g, 63 mmol, 2 eq) and stirred at 25° C. for 30 min, then 3-bromoprop-1-ene (7.7 g, 63 mmol, 2 eq) was added in it and stirred at 25° C. for 5 h to give a white suspension. The reaction mixture was diluted with water (300 mL) and extracted with EtOAc (400 mL×3), the combined organic layers were washed with saturated brine (500 mL×3), dried over Na₂SO₄, filtered and concentrated under reduced pressure to give a residue. The residue was purified by column chromatography to give allyl 5-((diethoxyphosphoryl)fluoromethyl)benzo[b]thiophene-2-carboxylate (10 g, 26 mmol, 82% yield) as a white solid. ¹H NMR (400 MHz, CDCl₃) δ 8.10 (s, 1H), 8.05-7.98 (m, 1H), 7.91 (d, J=8.4 Hz, 1H), 7.59 (d, J=8.4 Hz, 1H), 6.13-5.97 (m, 1H), 5.91-5.71 (m, 1H), 5.52-5.40 (m, 1H), 5.33 (dd, J=1.2, 10.4 Hz, 1H), 4.86 (dd, J=1.2, 5.6 Hz, 2H), 4.19-4.03 (m, 4H), 1.33-1.24 (m, 6H).

### Step 4: Preparation of ((2-((allyloxy)carbonyl)benzo[b]thiophen-5-yl)fluoromethyl)phosphonic acid

To a solution of allyl 5-((diethoxyphosphoryl)fluoromethyl)benzo[b]thiophene-2-carboxylate (9 g, 23 mmol, 1 eq) and (E)-(trimethylsilyl 2,2,2-trifluoro-N-(trimethylsilyl)ethanimidate) (18 g, 70 mmol, 3 eq) in DCM (100 mL) was added iodotrimethylsilane (16 g, 93 mmol, 4 eq) at 0° C. and stirred at 0° C. for 1 h to give a brown solution. The reaction mixture was concentrated under reduced pressure to give a residue. The crude was purified by reverse phase chromatography (TFA) then lyophilization to give ((2-((allyloxy)carbonyl)benzo[b]thiophen-5-yl)fluoromethyl)phosphonic acid (6.0 g, 18 mmol, 78% yield) as a yellow solid. ¹H NMR (400 MHz, DMSO-d₆) δ 8.31 (s, 1H), 8.14-8.05 (m, 2H), 7.59 (d, J=8.4 Hz, 1H), 6.13-5.98 (m, 1H), 5.90-5.76 (m, 1H), 5.44 (dd, J=1.6, 17.2 Hz, 1H), 5.31 (dd, J=1.6, 10.4 Hz, 1H), 4.86-4.83 (m, 2H).

### Step 5: Preparation of allyl 5-((dichlorophosphoryl)fluoromethyl)benzo[b]thiophene-2-carboxylate

To a solution of ((2-((allyloxy)carbonyl)benzo[b]thiophen-5-yl)fluoromethyl)phosphonic acid (2 g, 6.0 mmol, 1 eq) and DMF (88 mg, 1.2 mmol, 0.2 eq) in DCM (20 mL) was added oxalic dichloride (2.7 g, 21 mmol, 3.5 eq) at 0° C. and stirred at 40° C. for 0.5 h to give a yellow solution. The reaction mixture was concentrated under reduced pressure to give allyl 5-((dichlorophosphoryl)fluoromethyl)benzo[b]thiophene-2-carboxylate (2.2 g, crude) as a yellow solid. LCMS (ESI) m/z=717.1 (MeOH quench)

### Step 6: Preparation of allyl 5-(fluoro((((S)-1-oxo-1-propoxypropan-2-yl)amino)(phenoxy)phosphoryl)methyl)benzo[b]thiophene-2-carboxylate

To a solution of allyl 5-((dichlorophosphoryl)fluoromethyl)benzo[b]thiophene-2-carboxylate (2.2 g, 6.0 mmol, 1 eq) in DCM (20 mL), the solution was under N₂ and cooled to 0° C., then phenol (0.56 g, 6.0 mmol, 1 eq) in DCM (10 mL) was added in it, ethylbis(propan-2-yl)amine (3.1 g, 24 mmol, 4 eq) in DCM (60 mL) was dropwise added in it and stirred at 0° C. for 5 min, 3,3,3-trifluoropropyl (2S)-2-aminopropanoate (0.91 g, 6.0 mmol, 1 eq) in DCM (20 mL) was added in it and stirred at 0° C. for 10 min to give a yellow solution. The reaction mixture was concentrated under reduced pressure to give a residue. The residue was purified by column chromatography to give allyl 5-(fluoro((((S)-1-oxo-1-propoxypropan-2-yl)amino)(phenoxy)phosphoryl)methyl)benzo[b]thiophene-2-carboxylate (1.1 g, 1.9 mmol, 33% yield) as a white solid. ¹H NMR (400 MHz, CDCl₃) δ 8.11-8.07 (m, 1H), 8.05-7.99 (m, 1H), 7.93-7.86 (m, 1H), 7.67-7.58 (m, 1H), 7.40-7.32 (m, 1H), 7.27-7.17 (m, 2H), 7.15-7.05 (m, 2H), 6.10-5.89 (m, 2H), 5.50-5.42 (m, 1H), 5.34 (dd, J=1.2, 10.4 Hz, 1H), 4.87 (d, J=5.6 Hz, 2H), 4.22-4.08 (m, 1H), 4.00-3.76 (m, 1H), 3.69-3.51 (m, 1H), 1.67-1.59 (m, 2H), 1.33-1.26 (m, 3H), 0.96-0.89 (m, 3H).

### Step 7: Preparation of 5-(fluoro((((S)-1-oxo-1-propoxypropan-2-yl)amino)(phenoxy)phosphoryl)methyl)benzo[b]thiophene-2-carboxylic acid

To a solution of allyl 5-(fluoro((((S)-1-oxo-1-propoxypropan-2-yl)amino)(phenoxy)phosphoryl)methyl)benzo[b]thiophene-2-carboxylate (1 g, 1.9 mmol, 1 eq) and pyrrolidine (82 mg, 1.2 mmol, 0.6 eq) in DCM (10 mL) was added Pd(PPh₃)₄ (0.22 g, 0.19 mmol, 0.1 eq) and stirred at 25° C. for 1 h to give a yellow solution. The reaction mixture was concentrated under reduced pressure to give a residue. The crude was purified by reverse phase chromatography (TFA) then lyophilized to give 5-(fluoro((((S)-1-oxo-1-propoxypropan-2-yl)amino)(phenoxy)phosphoryl)methyl)benzo[b]thiophene-2-carboxylic acid (0.70 g, 1.5 mmol, 76% yield) as a white solid. ¹H NMR (400 MHz, CDCl₃) δ 8.15-7.91 (m, 1H), 7.90-7.79 (m, 2H), 7.70-7.51 (m, 1H), 7.46-7.29 (m, 3H), 7.25-7.15 (m, 2H), 6.15-5.98 (m, 1H), 4.24-4.05 (m, 3H), 1.79-1.58 (m, 2H), 1.41-1.22 (m, 3H), 0.96 (dd, J=12.4, 19.6 Hz, 3H).

### Step 8: Preparation of perfluorophenyl 5-(fluoro((((S)-1-oxo-1-propoxypropan-2-yl)amino)(phenoxy)phosphoryl)methyl)benzo[b]thiophene-2-carboxylate

To a solution of 5-(fluoro((((S)-1-oxo-1-propoxypropan-2-yl)amino)(phenoxy)phosphoryl)methyl)benzo[b]thiophene-2-carboxylic acid (0.70 g, 1.5 mmol, 1 eq) and pyridine (0.57 g, 7.2 mmol, 5 eq) in DMF (10 mL) was added 2,3,4,5,6-pentafluorophenyl 2,2,2-trifluoroacetate (2.0 g, 7.2 mmol, 5 eq) and stirred at 25° C. for 0.5 h to give a yellow solution. The reaction mixture was diluted with water (50 mL) and extracted with EtOAc (50 mL×2), the combined organic layers were washed with saturated brine (40 mL×2), dried over Na₂SO₄, filtered and concentrated under reduced pressure to give a residue. The residue was purified by column chromatography to give perfluorophenyl 5-(fluoro((((S)-1-oxo-1-propoxypropan-2-yl)amino)(phenoxy)phosphoryl)methyl)benzo[b]thiophene-2-carboxylate (0.80 g, 1.2 mmol, 86% yield) as a white solid. LCMS (ESI) m/z=646.1 [M+H]⁺. ¹H NMR (400 MHz, CDCl₃) δ 8.38-8.30 (m, 1H), 8.15-8.07 (m, 1H), 8.02-7.91 (m, 1H), 7.78-7.63 (m, 1H), 7.43-7.34 (m, 1H), 7.31-7.28 (m, 1H), 7.25-7.17 (m, 1H), 7.16-7.06 (m, 2H), 6.13-5.89 (m, 1H), 4.11-4.01 (m, 2H), 3.70-3.51 (m, 1H), 1.72-1.59 (m, 3H), 1.32 (d, J=7.2 Hz, 2H), 0.99-0.87 (m, 3H).

### Synthesis of ((2-((perfluorophenoxy)carbonyl)benzo[b]thiophen-5-yl)methyl)phosphonic acid

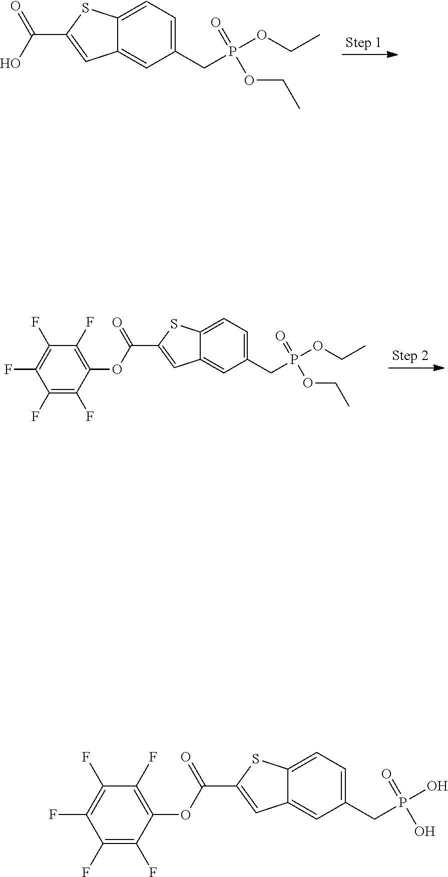

### Step 1: Preparation of ((2-((perfluorophenoxy)carbonyl)benzo[b]thiophen-5-yl)methyl)phosphonic acid

To a solution of 5-((diethoxyphosphoryl)methyl)benzo[b]thiophene-2-carboxylic acid (2 g, 6.1 mmol, 1 eq) in dimethylformamide (100 mL) was added pyridine (1.9 g, 24 mmol, 4 eq), the mixture was stirred at 0° C. for 5 min, then 2,3,4,5,6-pentafluorophenyl 2,2,2-trifluoroacetate (6.8 g, 24 mmol, 4 eq) was added to the mixture, the mixture was stirred at 25° C. for 15 min to give yellow solution. The mixture was diluted with water (50 mL) and extracted with EtOAc (50 mL×2), the combined organic layers were washed with saturated brine (40 mL×2), dried over Na₂SO₄, filtered and concentrated under reduced pressure to give a residue. The residue was purified by column chromatography to give perfluorophenyl 5-((diethoxyphosphoryl)methyl)benzo[b]thiophene-2-carboxylate (1.4 g, 2.9 mmol, 47% yield) as a yellow oil. LCMS (ESI) m/z=495.0

### Step 2: Preparation of ((2-((perfluorophenoxy)carbonyl)benzo[b]thiophen-5-yl)methyl)phosphonic acid

To a solution of perfluorophenyl 5-((diethoxyphosphoryl)methyl)benzo[b]thiophene-2-carboxylate (1.4 g, 2.8 mmol, 1 eq) in DCM (10 mL) was added trimethylsilyl iodide (2.3 g, 11 mmol, 4 eq), the mixture was stirred at 0° C. for 15 min to give yellow solution. The reaction mixture was concentrated under reduced pressure to give a residue. The residue was purified by reversed phase (TFA) to give ((2-((perfluorophenoxy)carbonyl)benzo[b]thiophen-5-yl)methyl)phosphonic acid (0.9 g, 2.1 mmol, 74% yield) as a white solid. LCMS (ESI) m/z=438.9 [M+H]⁺. ¹H NMR (400 MHz, CD₃OD) δ 8.41 (s, 1H), 8.01-7.94 (m, 2H), 7.60-7.55 (m, 1H), 3.32-3.32 (m, 1H), 3.26 (s, 1H).

### Synthesis of (fluoro(2-((perfluorophenoxy)carbonyl)benzo[b]thiophen-5-yl)methyl)phosphonic acid

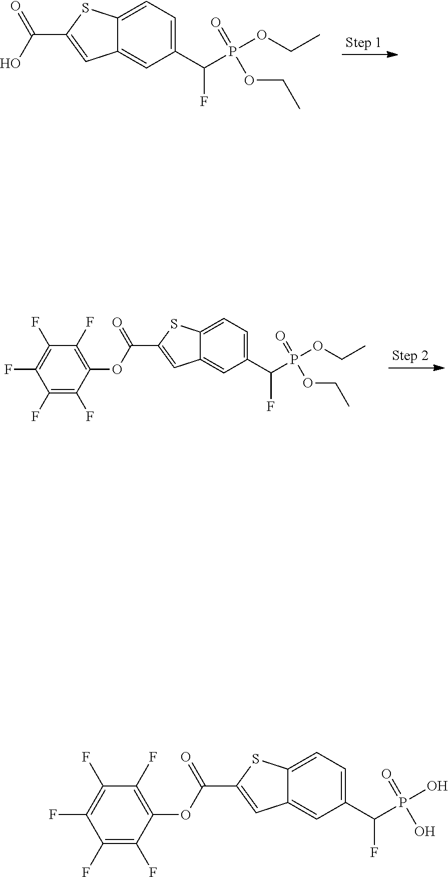

### Step 1: Preparation of perfluorophenyl 5-((diethoxyphosphoryl)fluoromethyl)benzo[b]thiophene-2-carboxylate

To a solution of 5-[(diethoxyphosphoryl)(fluoro)methyl]-1-benzothiophene-2-carboxylic acid (5 g, 14.40 mmol, 1 eq) in piperidine (6.13 g, 72 mmol, 5 eq) was added 2,3,4,5,6-pentafluorophenyl 2,2,2-trifluoroacetate (12 g, 43.20 mmol, 3 eq), the mixture was stirred at 0° C. for 30 min to give a brown clean solution. The reaction mixture was concentrated under reduced pressure to give a residue. The residue was diluted with water (200 mL) and extracted with EtOAc (500 mL×2), the combined organic layers were washed with saturated brine (500 mL×2), dried over Na₂SO₄, filtered and concentrated under reduced pressure to give a residue. The residue was purified by flash silica gel chromatography to give perfluorophenyl 5-((diethoxyphosphoryl)fluoromethyl)benzo[b]thiophene-2-carboxylate (6.50 g, 11.70 mmol, 81.40%) as a yellow oil. LCMS (ESI) m/z=513.1 [M+H]⁺. ¹H NMR (400 MHz, CDCl₃) δ 8.35 (s, 1H), 8.08 (s, 1H), 7.98 (d, J=8.4 Hz, 1H), 7.67 (d, J=8.4 Hz, 1H), 5.93-5.77 (m, 1H), 4.22-4.17 (m, 1H), 4.16-4.13 (m, 1H), 4.13-4.11 (m, 1H), 4.10-4.06 (m, 1H), 1.34-1.31 (m, 3H), 1.30-1.27 (m, 3H).

### Step 2: Preparation of (fluoro(2-((perfluorophenoxy)carbonyl)benzo[b]thiophen-5-yl)methyl)phosphonic acid

To a solution of perfluorophenyl 5-((diethoxyphosphoryl)fluoromethyl)benzo[b]thiophene-2-carboxylate (6 g, 12 mmol, 1 eq) in DCM (60 mL) was dropwise TMSI (9.3 g, 47 mmol, 4 eq) at 0 C, the mixture was stirred at 0° C. for 1 h. The reaction mixture was concentrated under reduced pressure to give a yellow oil. The oil was purified by reversed phase (TFA) to lyophilized to give (fluoro(2-((perfluorophenoxy)carbonyl)benzo[b]thiophen-5-yl)methyl)phosphonic acid (3.0 g, 6.6 mmol, 56% yield) as a white solid. LCMS (ESI) m/z=457.2 [M+H]⁺. ¹H NMR (400 MHz, CD₃OD) δ 8.48 (s, 1H), 8.17 (s, 1H), 8.07 (d, J=8.4 Hz, 1H), 7.73 (d, J=8.4 Hz, 1H), 5.97-5.78 (m, 1H).

### Synthesis of 5-((1S)-fluoro(phenoxy((S)-2-(propoxycarbonyl)pyrrolidin-1-yl)phosphoryl)methyl)benzo[b]thiophene-2-carboxylic acid

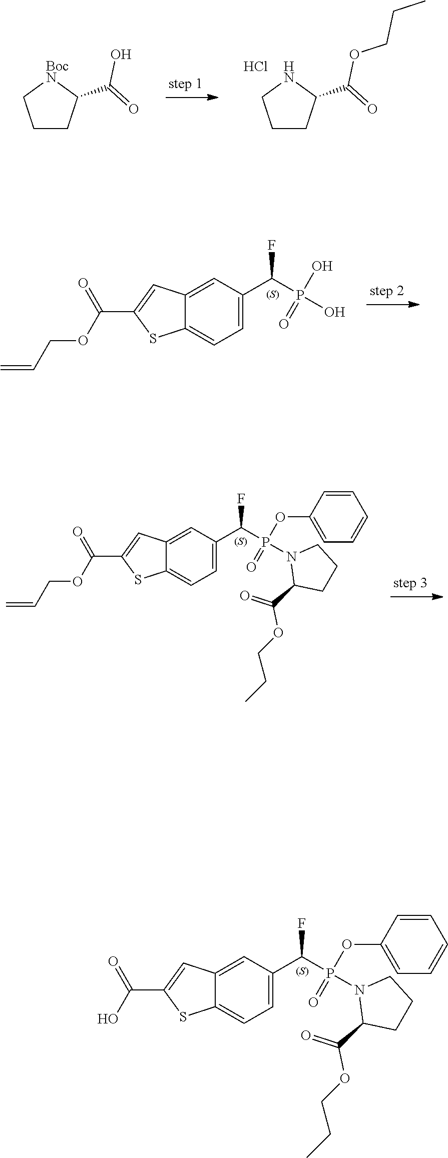

### Step 1: Preparation of propyl L-prolinate hydrochloride

Thionyl chloride (2.01 mL, 27.8 mmol, 3 eq) was added dropwise to a solution of (tert-butoxycarbonyl)-L-proline (2.00 g, 9.29 mmol, 1 eq) in propan-1-ol (20 mL, 332 μmol, eq) at 0° C. The mixture was allowed to warm to ambient temperature and then heated at 80° C. overnight. The reaction mixture was concentrated under reduced pressure to give crude propyl L-prolinate hydrochloride (1.85 g, 103%) as a brown oil. ¹H NMR (400 MHz, DMSO-d₆) δ 4.36-4.33 (m, 1H), 4.18-4.07 (m, 2H), 3.26-3.14 (m, 2H), 2.32-2.22 (m, 1H), 2.01-1.85 (m, 3H), 1.66-1.59 (m, 2H), 0.92-0.89 (m, 3H).

The intermediates in the table below were prepared using the method described above in Step 1 for the preparation of propyl L-prolinate hydrochloride and utilizing the appropriate starting materials and modifications.

| Name | Structure | LCMS |
|---|---|---|
| propyl (S)-azetidine-2- carboxylate (HCl salt) | 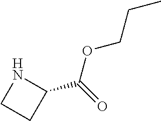 | 144.2 [M + H]⁺ |

### Step 2: Preparation of propyl (((S)-(2-((allyloxy)carbonyl)benzo[b]thiophen-5-yl)fluoromethyl)(phenoxy)phosphoryl)-L-prolinate

To a solution of (S)-((2-((allyloxy)carbonyl)benzo[b]thiophen-5-yl)fluoromethyl)phosphonic acid (500 mg, 1.51 mmol, 1 eq) and DMF (2 drops) in DCM (15 mL) was added dropwise oxalyl chloride (1.02 mL, 12.0 mmol, 8 eq). The reaction mixture was stirred 2 h at room temperature under N₂. The reaction mixture was concentrated under reduced pressure and dried completely under high vacuum for 30 min. to give a yellow solid. The yellow solid was diluted in DCM (15 mL, dried on Na₂SO₄) and cooled down to 0° C. A solution of phenol (134 mg, 1.43 mmol, 0.95 eq) and triethylamine (1.26 mL, 9.06 mmol, 6.0 eq) in DCM (5 mL, dried on Na₂SO₄) was slowly added onto the solution. The reaction mixture was stirred at 0° C. for 2 min. and then warmed up to room temperature and stirred for 18 h. A solution of propyl L-prolinate hydrochloride (584 mg, 3.02 mmol, 2 eq) in DCM (5 mL, dried on Na₂SO₄) was slowly added onto the orange solution. The reaction mixture was stirred at room temperature for 4 h. Water (1 mL) was added. The solvent was removed under reduced pressure. The crude residue was purified by reverse phase chromatography on a 150 g Cis cartridge eluting with 5-100% MeCN in water. The pure fractions were then concentrated under reduced pressure to give propyl (((S)-(2-((allyloxy)carbonyl)benzo[b]thiophen-5-yl)fluoromethyl)(phenoxy)phosphoryl)-L-prolinate (320 mg, 38.8%) as a yellow oil. LCMS (ESI): m/z=546.2 [M+H]⁺. ¹H NMR (400 MHz, CDCl₃) δ 8.17-8.04 (m, 2H), 7.94-7.86 (m, 1H), 7.79-7.62 (m, 1H), 7.34-7.28 (m, 2H), 7.24-7.20 (m, 1H), 7.19-7.03 (m, 2H), 6.10-6.00 (m, 1H), 5.49-5.31 (m, 2H), 4.89-4.84 (m, 2H), 4.57-4.22 (m, 1H), 4.15-3.96 (m, 2H), 3.66-3.21 (m, 1H), 3.13-2.67 (m, 1H), 2.18-2.04 (m, 1H), 1.97-1.86 (m, 1H), 1.83-1.46 (m, 5H), 1.00-0.86 (m, 3H).

The intermediates in the table below were prepared using the method described above in Step 2 for the preparation of propyl (((S)-(2-((allyloxy)carbonyl)benzo[b]thiophen-5-yl)fluoromethyl)(phenoxy)phosphoryl)-L-prolinate and utilizing the appropriate starting materials and modifications.

| Name | Structure | LCMS |
|---|---|---|
| propyl (2S)-1-(((S)-(2- ((allyloxy)carbonyl)benzo[b]thiophen- 5- yl)fluoromethyl)(phenoxy)phosphoryl) azetidine-2-carboxylate | 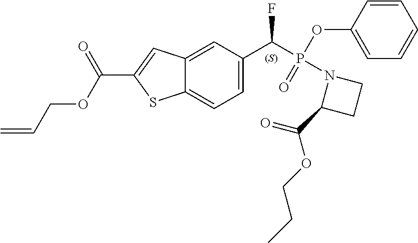 | 532.2 [M + H]⁺ |

### Step 3: Preparation of 5-((1S)-fluoro(phenoxy((S)-2-(propoxycarbonyl)pyrrolidin-1-yl)phosphoryl)methyl)benzo[b]thiophene-2-carboxylic acid

To a solution of propyl (((S)-(2-((allyloxy)carbonyl)benzo[b]thiophen-5-yl)fluoromethyl)(phenoxy)phosphoryl)-L-prolinate (320 mg, 586 μmol, 1 eq) in THF (6 mL) were added morpholine (251 μL, 2.92 mmol, 5 eq) and Pd(PPh₃)₄ (13.5 mg, 11.7 μmol, 0.02 eq). The reaction mixture was stirred at room temperature for 1 h. The reaction mixture was concentrated to a volume of 2 mL under N₂. The crude product was purified by reverse phase chromatography on a 100 g Cis cartridge eluting with 5-80% MeCN in water (with 0.1% formic acid). The combined fractions were concentrated under reduced pressure and then freeze dried to give 5-((1S)-fluoro(phenoxy((S)-2-(propoxycarbonyl)pyrrolidin-1-yl)phosphoryl)methyl)benzo[b]thiophene-2-carboxylic acid (172 mg) as a white solid which was re-purified by reverse phase chromatography on a 275 g Cis cartridge eluting with 5-80% MeCN in water (with 0.1% formic acid). The combined fractions were concentrated under reduced pressure and then freeze dried to give 5-((1S)-fluoro(phenoxy((S)-2-(propoxycarbonyl)pyrrolidin-1-yl)phosphoryl)methyl)benzo[b]thiophene-2-carboxylic acid (75.0 mg, 25.3%) as a white solid. LCMS (ESI): m/z=506.0 [M+H]⁺. ¹H NMR (400 MHz, DMSO-d₆) δ 13.58 (br. s., 1H), 8.21-8.10 (m, 2H), 7.73-7.63 (m, 1H), 7.41-7.31 (m, 3H), 7.30-7.16 (m, 2H), 7.12-7.08 (m, 1H), 6.86-6.28 (m, 1H), 4.43-3.41 (m, 4H), 3.19-2.82 (m, 1H), 2.10-1.61 (m, 4H), 1.57-1.45 (m, 2H), 0.92-0.72 (m, 3H).

The intermediates in the table below were prepared using the method described above in Step 3 for the preparation of 5-((1S)-fluoro(phenoxy((S)-2-(propoxycarbonyl)pyrrolidin-1-yl)phosphoryl)methyl)benzo[b]thiophene-2-carboxylic acid and utilizing the appropriate starting materials and modifications.

| Name | Structure | LCMS |
|---|---|---|
| 5-((1S)-fluoro(phenoxy((S)-2- (propoxycarbonyl)azetidin-1- yl)phosphoryl)methyl)benzo[b]thiophene- 2-carboxylic acid | 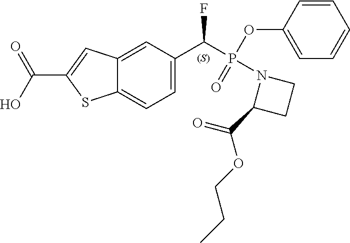 | 492.2 [M + H]⁺ |

### Synthesis of 5-((1R)-fluoro((((S)-1-(2-methoxy-2-methylpropoxy)-1-oxopropan-2-yl)amino)(phenoxy)phosphoryl)methyl)benzo[b]thiophene-2-carboxylic acid

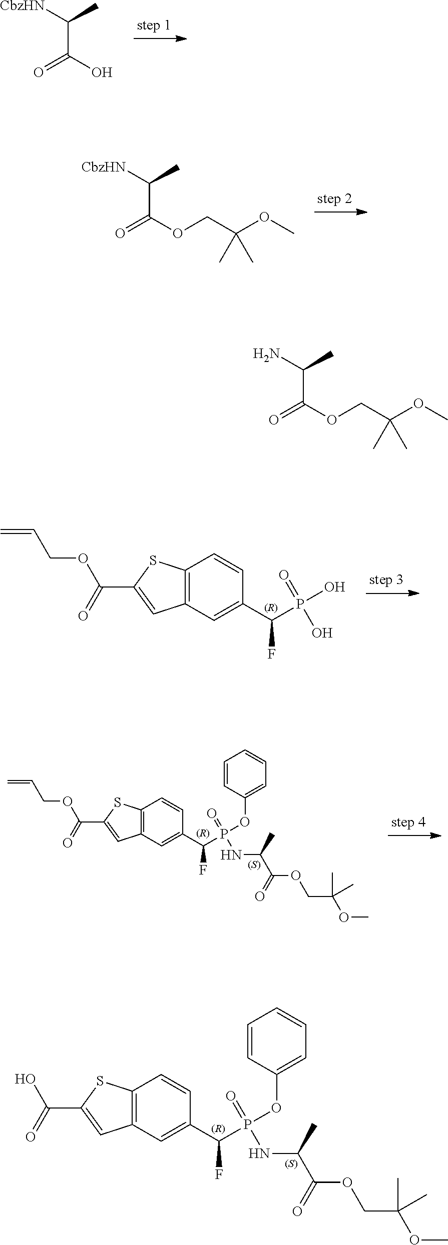

### Step 1: Preparation of 2-methoxy-2-methylpropyl ((benzyloxy)carbonyl)-L-alaninate

To a solution of ((benzyloxy)carbonyl)-L-alanine (1.64 g, 7.34 mmol, 1 eq.) and 2-methoxy-2-methylpropan-1-ol (916 mg, 8.80 mmol, 1.2 eq.) in ACN (20 ml) were added EDCI (1.68 g, 8.80 mmol, 1.2 eq.) and DMAP (1.07 g, 8.80 mmol, 1.2 eq.). The mixture was stirred at 25° C. for 16 h. After completion, the reaction mixture was diluted with H₂O (10 mL) and extracted with DCM (10 mL×3). The organic layers were combined and washed with brine (10 mL), dried over anhydrous Na₂SO₄, and concentrated under reduced pressure. The residue was purified by flash column chromatography on silica gel to afford 2-methoxy-2-methylpropyl ((benzyloxy)carbonyl)-L-alaninate (2, 1.63 g, 5.27 mmol, 72%) as a colorless oil. LCMS (ESI): m/z=310 [M+H]⁺.

The intermediates in the table below were prepared using the method described above in Step 1 for the preparation of 2-methoxy-2-methylpropyl ((benzyloxy)carbonyl)-L-alaninate and utilizing the appropriate starting materials and modifications.

| Name | Structure | LCMS |
|---|---|---|
| (tetrahydro-2H-pyran-4-yl)methyl ((benzyloxy)carbonyl)-L-alaninate | 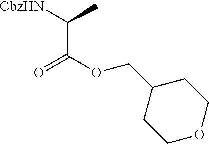 [SMILES: C[C@H](NC(=O)OCc1ccccc1)C(=O)OCC1CCOCC1] | 322 [M + H]⁺ |
| pyridazin-3-ylmethyl ((benzyloxy)carbonyl)-L-alaninate | 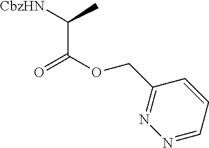 | 282 [M + H]⁺ |
| oxetan-3-ylmethyl((benzyloxy)carbonyl)- L-alaninate | 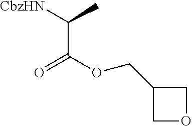 [SMILES: C[C@H](NC(=O)OCc1ccccc1)C(=O)OCC1COC1] | 294 [M + H]⁺ |
| (3-methyloxetan-3-yl)methyl ((benzyloxy)carbonyl)-L-alaninate | 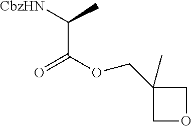 [SMILES: C[C@H](NC(=O)OCc1ccccc1)C(=O)OCC1(C)COC1] | 308 [M + H]⁺ |

### Step 2: Preparation of 2-methoxy-2-methylpropyl L-alaninate

To a solution of 2-methoxy-2-methylpropyl ((benzyloxy)carbonyl)-L-alaninate (1.5 g, 4.84 mmol, 1.0 eq.) in EtOAc (15 ml) was added Pd/C (150 mg) under nitrogen. The suspension was degassed under vacuum and purged with H₂ several times. The resulting mixture was stirred at room temperature for 14 h. After completion, the suspension was filtered through a pad of Celite®, the filter cake was washed with MeOH (20 mL). The combined filtrates were concentrated to dryness to give 2-methoxy-2-methylpropyl L-alaninate (640 mg, 3.65 mmol, 75%) as a colorless oil. LCMS (ESI): m/z=176 [M+H]⁺.

The intermediates in the table below were prepared using the method described above in Step 2 for the preparation of 2-methoxy-2-methylpropyl ((benzyloxy)carbonyl)-L-alaninate and utilizing the appropriate starting materials and modifications.

| Name | Structure | LCMS |
|---|---|---|
| (tetrahydro-2H-pyran-4-yl)methyl L-alaninate | 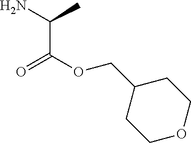 | 188 [M + H]⁺ |
| pyridazin-3-ylmethyl L-alaninate | 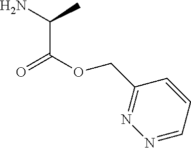 | 182 [M + H]⁺ |
| oxetan-3-ylmethyl L-alaninate | 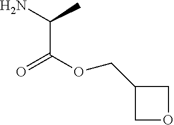 | 160 [M + H]⁺ |
| (3-methyloxetan-3-yl)methyl L-alaninate | 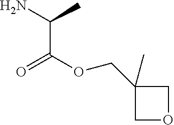 | 147.1 [M + H]⁺ |

### Step 3: Preparation of allyl 5-((1R)-fluoro((((S)-1-(2-methoxy-2-methylpropoxy)-1-oxopropan-2-yl)amino)(phenoxy)phosphoryl)methyl)benzo[b]thiophene-2-carboxylate

Oxalyl chloride (576 mg, 4.54 mmol, 5 eq.) was added dropwise to the solution of (R)-((2-((allyloxy)carbonyl)benzo[b]thiophen-5-yl)fluoromethyl)phosphonic acid (300 mg, 0.91 mmol, 1 eq.) in dry DCM (10 mL) and DMF (1 drop) at 25° C. The reaction mixture was stirred at 40° C. for an additional 0.5 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (10 mL), then added to a mixture of phenol (88.8 mg, 0.94 mmol, 1.05 eq.) and TEA (908 mg, 8.98 mmol, 10.0 eq.) in anhydrous DCM (5 mL) at 0° C. The reaction was stirred for 0.5 h. Then 2-methoxy-2-methylpropyl L-alaninate (313 mg, 1.79 mmol, 2 eq.) was added at 0° C. and the resulting mixture was stirred at 20° C. for 1 h under N₂. After completion, the reaction mixture was concentrated under reduced pressure. The residue was purified by Biotage® C18 column chromatography to give allyl 5-((1R)-fluoro((((S)-1-(2-methoxy-2-methylpropoxy)-1-oxopropan-2-yl)amino)(phenoxy)phosphoryl)methyl)benzo[b]thiophene-2-carboxylate (45.0 mg, 80 μmol, 9%) as a yellow solid. LCMS (ESI): m/z=564.0 [M+H]⁺.

The intermediates in the table below were prepared using the method described above in Step 3 for the preparation of allyl 5-((1R)-fluoro((((S)-1-(2-methoxy-2-methylpropoxy)-1-oxopropan-2-yl)amino)(phenoxy)phosphoryl)methyl)benzo[b]thiophene-2-carboxylate and utilizing the appropriate starting materials and modifications.

| Name | Structure | LCMS |
|---|---|---|
| allyl 5-((1R)-fluoro((((S)-1-oxo-1- ((tetrahydro-2H-pyran-4-yl)methoxy)propan- 2- yl)amino)(phenoxy)phosphoryl)methyl)benzo[b] thiophene-2-carboxylate | 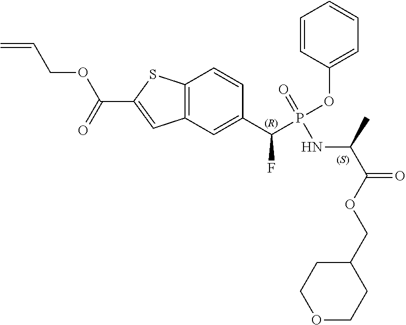 | 576 [M + H]⁺ |
| allyl 5-((1R)-fluoro((((S)-1-oxo-1-(pyridazin- 3-ylmethoxy)propan-2- yl)amino)(phenoxy)phosphoryl)methyl)benzo[b] thiophene-2-carboxylate | 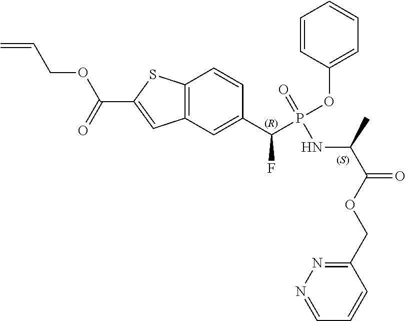 | 570 [M + H]⁺ |
| allyl 5-((1R)-fluoro((((S)-1-(oxetan-3- ylmethoxy)-1-oxopropan-2- yl)amino)(phenoxy)phosphoryl)methyl)benzo[b] thiophene-2-carboxylate | 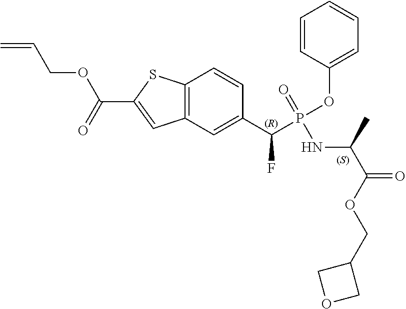 | 548 [M + H]⁺ |
| allyl 5-((1R)-fluoro((((S)-1-((3-methyloxetan- 3-yl)methoxy)-1-oxopropan-2- yl)amino)(phenoxy)phosphoryl)methyl)benzo[b] thiophene-2-carboxylate | 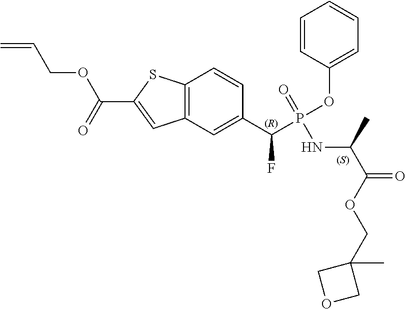 | 562 [M + H]⁺ |

### Step 4: Preparation of 5-((1R)-fluoro((((S)-1-(2-methoxy-2-methylpropoxy)-1-oxopropan-2-yl)amino)(phenoxy)phosphoryl)methyl)benzo[b]thiophene-2-carboxylic acid

To a solution of allyl 5-((1R)-fluoro((((S)-1-(2-methoxy-2-methylpropoxy)-1-oxopropan-2-yl)amino)(phenoxy)phosphoryl)methyl)benzo[b]thiophene-2-carboxylate (45.0 mg, 80 μmol, 1.0 eq) in DCM (6 mL) were added Pd(PPh₃)₄ (5 mg, 8 μmol, 0.1 eq.) and pyrrolidine (6 mg, 80 μmol, 1.0 eq.). The mixture was purged and degassed with N₂ (three times), then stirred at 25° C. for 1 hr. After completion, the mixture was concentrated under reduced pressure. The residue was purified by Biotage® C18 column chromatography to give 5-((1R)-fluoro((((S)-1-(2-methoxy-2-methylpropoxy)-1-oxopropan-2-yl)amino)(phenoxy)phosphoryl)methyl)benzo[b]thiophene-2-carboxylic acid (40 mg, 76 μmol, 95%) as a colorless oil. LCMS (ESI): m/z=524 [M+H]⁺.

The intermediates in the table below were prepared using the method described above in Step 4 for the preparation of 5-((1R)-fluoro((((S)-1-(2-methoxy-2-methylpropoxy)-1-oxopropan-2-yl)amino)(phenoxy)phosphoryl)methyl)benzo[b]thiophene-2-carboxylic acid and

| Name | Structure | LCMS |
|---|---|---|
| 5-((1R)-fluoro((((S)-1-oxo-1-((tetrahydro-2H- pyran-4-yl)methoxy)propan-2- yl)amino)(phenoxy)phosphoryl)methyl)benzo[b] thiophene-2-carboxylic acid | 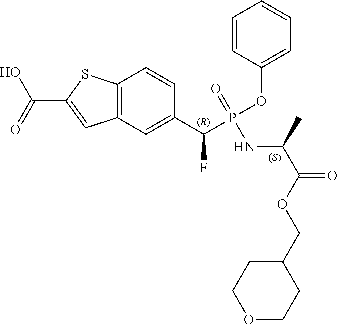 | 536 [M + H]⁺ |
| 5-((1R)-fluoro((((S)-1-oxo-1-(pyridazin-3- ylmethoxy)propan-2- yl)amino)(phenoxy)phosphoryl)methyl)benzo[b] thiophene-2-carboxylic acid | 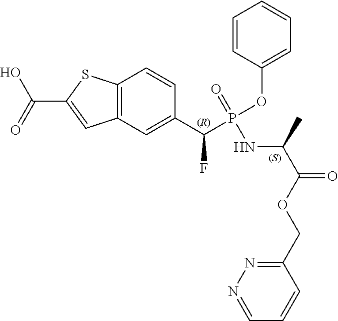 | 530 [M + H]⁺ |
| 5-((1R)-fluoro((((S)-1-(oxetan-3-ylmethoxy)- 1-oxopropan-2- yl)amino)(phenoxy)phosphoryl)methyl)benzo[b] thiophene-2-carboxylic acid | 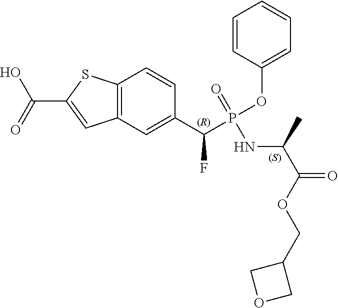 | 508 [M + H]⁺ |
| 5-((1R)-fluoro((((S)-1-((3-methyloxetan-3- yl)methoxy)-1-oxopropan-2- yl)amino)(phenoxy)phosphoryl)methyl)benzo[b] thiophene-2-carboxylic acid | 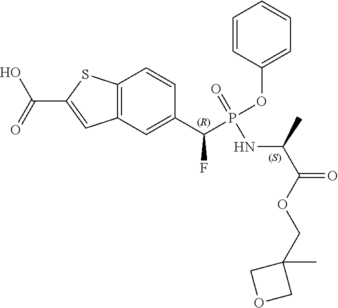 | 522 [M + H]⁺ |

### Synthesis of 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylic acid

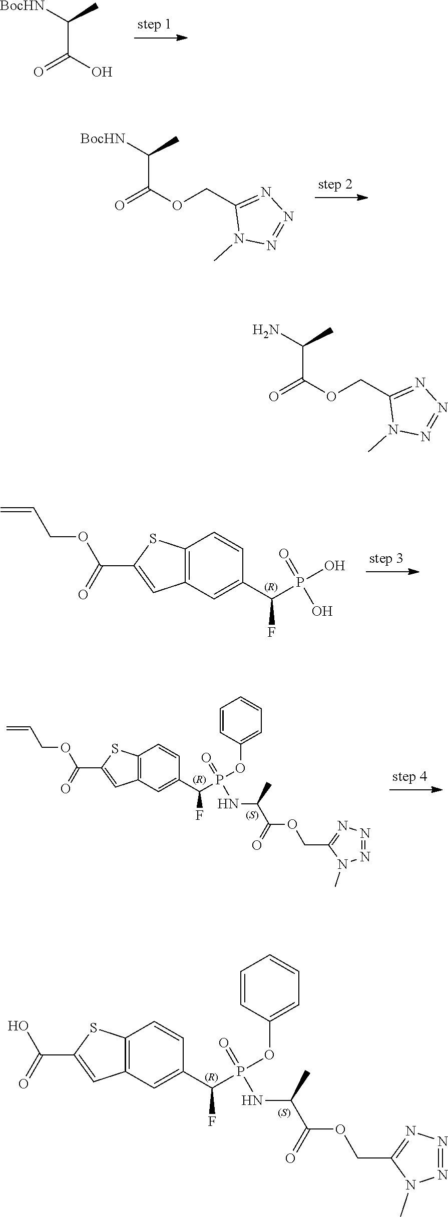

### Step 1: Preparation of (1-methyl-1H-tetrazol-5-yl)methyl (tert-butoxycarbonyl)-L-alaninate

To a solution of (tert-butoxycarbonyl)-L-alanine (1 g, 5.28 mmol, 1 eq.) and (1-methyl-1H-1,2,3,4-tetrazol-5-yl)methanol (903 mg, 7.92 mmol, 1.5 eq.) in DCM (15 ml) were added T₃P (50%) (5.02 g, 15.8 mmol, 3 eq.), DMAP (32.2 mg, 264 μmol, 0.05 eq.) and 4-methylmorpholine (1.59 g, 15.8 mmol, 3 eq.). The mixture was stirred at room temperature for 2 h. After completion, the reaction mixture was diluted with H₂O (10 mL) and extracted with DCM (10 mL×3). The organic layers were combined and washed with brine (10 mL), dried over anhydrous Na₂SO₄, and concentrated under reduced pressure. The residue was purified by flash column chromatography on silica gel to afford (1-methyl-1H-tetrazol-5-yl)methyl (tert-butoxycarbonyl)-L-alaninate (900 mg, 3.15 mmol, 60%) as a colorless oil. LCMS (ESI): m/z=286 [M+H]⁺.

The intermediates in the table below were prepared using the method described above in Step 1 for the preparation of (1-methyl-1H-tetrazol-5-yl)methyl (tert-butoxycarbonyl)-L-alaninate and utilizing the appropriate starting materials and modifications.

| Name | Structure | LCMS | NMR |
|---|---|---|---|
| (4-methyl-4H-1,2,4-triazol- 3-yl)methyl(tert- butoxycarbonyl)-L- alaninate | 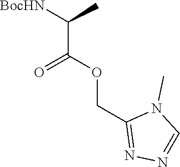 [SMILES: C[C@H](NC(=O)OC(C)(C)C)C(=O)OCc1nncn1C] | 285 [M + H]⁺ | |
| 2-methoxyethyl N-(tert- butoxycarbonyl)-O-methyl- L-serinate | 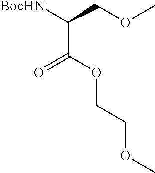 [SMILES: COCCOC(=O)[C@H](COC)NC(=O)OC(C)(C)C] | 278 [M + H]⁺ | ¹H NMR (400 MHz, DMSO-d₆) δ 7.19- 7.07 (m, 1H), 4.28- 4.10 (m, 3H), 3.61- 3.46 (m, 4H), 3.28- 3.19 (m, 6H), 1.38 (s, 9H). |
| 2-methoxyethyl(tert- butoxycarbonyl)-L- alaninate | 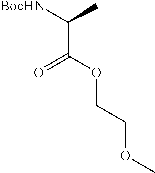 [SMILES: COCCOC(=O)[C@H](C)NC(=O)OC(C)(C)C] | 248 [M + H]⁺ | |
| propyl(tert- butoxycarbonyl)-L- alaninate | 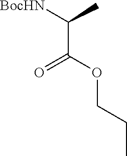 [SMILES: CCCOC(=O)[C@H](C)NC(=O)OC(C)(C)C] | | |
| propyl N-(tert- butoxycarbonyl)-O-methyl- L-serinate | 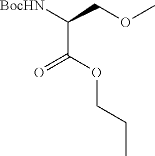 [SMILES: CCCOC(=O)[C@H](COC)NC(=O)OC(C)(C)C] | 262 [M + H]⁺ | |
| pyridin-2-ylmethyl(tert- butoxycarbonyl)-L- alaninate | 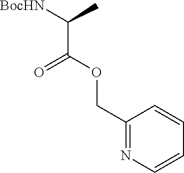 [SMILES: C[C@H](NC(=O)OC(C)(C)C)C(=O)OCc1ccccn1] | 281 [M + H]⁺ | |
| isobutyl(tert- butoxycarbonyl)-L- alaninate | 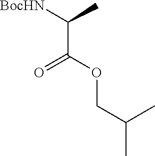 [SMILES: CC(C)COC(=O)[C@H](C)NC(=O)OC(C)(C)C] | 246 [M + H]⁺ | |
| cyclopropyl(tert- butoxycarbonyl)-L- alaninate | 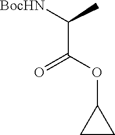 [SMILES: C[C@H](NC(=O)OC(C)(C)C)C(=O)OC1CC1] | 230 [M + H]⁺. | |
| neopentyl(tert- butoxycarbonyl)-L- alaninate | 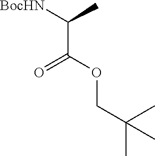 [SMILES: C[C@H](NC(=O)OC(C)(C)C)C(=O)OCC(C)(C)C] | 260 [M + H]⁺ | |

### Step 2: Preparation of (1-methyl-1H-tetrazol-5-yl)methyl L-alaninate

A solution of (1-methyl-1H-tetrazol-5-yl)methyl (tert-butoxycarbonyl)-L-alaninate (600 mg, 2.10 mmol, 1 eq) in HCl/EA (10 mL) was stirred at room temperature for 1 hr. After completion, the reaction mixture was concentrated under reduced pressure to give crude (1-methyl-1H-tetrazol-5-yl)methyl L-alaninate (390 mg, quant.)(HCl salt) as a white solid, which was used in next Step directly without further purification. LCMS (ESI): m/z=186 [M+H]⁺.

The intermediates in the table below were prepared using the method described above in Step 2 for the preparation of (1-methyl-1H-tetrazol-5-yl)methyl L-alaninate and utilizing the appropriate starting materials and modifications.

| Name | Structure | LCMS | NMR |
|---|---|---|---|
| (4-methyl-4H-1,2,4-triazol- 3-yl)methyl L-alaninate (HCl Salt) | 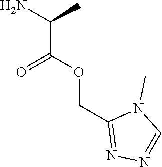 | 185 [M + H]⁺ | |
| 2-methoxyethyl O-methyl- L-serinate (HCl salt) | 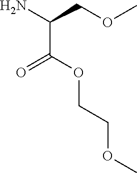 | | ¹H NMR (400 MHz, DMSO-d₆) δ 8.84 (s, 2H), 4.29-4.22 (m, 2H), 3.87-3.75 (m, 2H), 3.58-3.56 (m, 3H), 3.31-3.25 (m, 6H) |
| 2-methoxyethyl L-alaninate (HCl salt) | 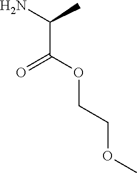 | 148 [M + H]⁺ | |
| propyl L-alaninate (HCl salt) | 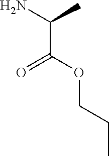 | | |
| propyl O-methyl-L-serinate (HCl salt) | 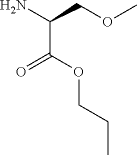 | 162 [M + H]⁺ | |
| pyridin-2-ylmethyl L- alaninate (HCl salt) | 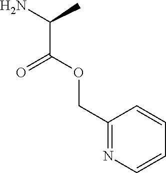 | 181 [M + H]⁺ | |
| isobutyl L-alaninate (HCl salt) | 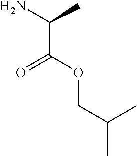 | 146 [M + H]⁺ | |
| cyclopropyl L-alaninate (HCl salt) | 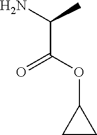 | 130 [M + H]⁺ | |
| benzyl L-alaninate (HCl salt) | 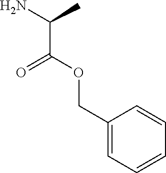 | 180 [M + H]⁺ | |
| neopentyl L-alaninate (HCl salt) | 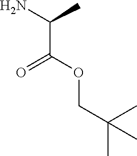 | 160 [M + H]⁺ | |

### Step 3: Preparation of allyl 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylate

Oxalyl chloride (1.05 g, 8.35 mmol, 5 eq.) was added dropwise to a solution of (R)-((2-((allyloxy)carbonyl)benzo[b]thiophen-5-yl)fluoromethyl)phosphonic acid (550 mg, 1.67 mmol, 1 eq.) in dry DCM (10 mL) and DMF (1 drop) at 25° C. The reaction mixture was stirred at 40° C. for an additional 0.5 hr. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (10 mL), then added to a mixture of phenol (146 mg, 1.56 mmol, 1.05 eq.) and TEA (752 mg, 7.45 mmol, 5 eq.) in anhydrous DCM (5 mL) at 0° C. The reaction was stirred for 0.5 h. Then (1-methyl-1H-tetrazol-5-yl)methyl L-alaninate (3, 551 mg, 2.98 mmol, 2 eq.) was added at 0° C. and the resulting mixture was stirred at 20° C. for 1 h under N₂. After completion, the reaction mixture was concentrated under reduced pressure. The residue was purified by Biotage® C18 column chromatography to give allyl 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylate (90.0 mg, 156 μmol, 11%) as a yellow oil. LCMS (ESI): m/z=574 [M+H]⁺.

The intermediates in the table below were prepared using the method described above in Step 3 for the preparation of allyl 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylate and utilizing the appropriate starting materials and modifications.

| Name | Structure | LCMS |
|---|---|---|
| allyl 5-((1R)-fluoro((((S)-1-((4- methyl-4H-1,2,4-triazol-3- yl)methoxy)-1-oxopropan-2- yl)amino)(phenoxy)phosphoryl)meth- yl)benzo[b]thiophene-2- carboxylate | 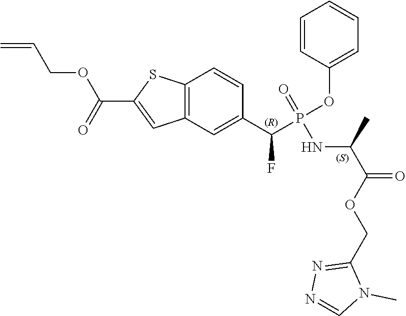 | 573 [M + H]⁺ |
| allyl 5-((1R)-fluoro((((S)-3- methoxy-1-(2-methoxyethoxy)-1- oxopropan-2- yl)amino)(phenoxy)phosphoryl)meth- yl)benzo[b]thiophene-2- carboxylate | 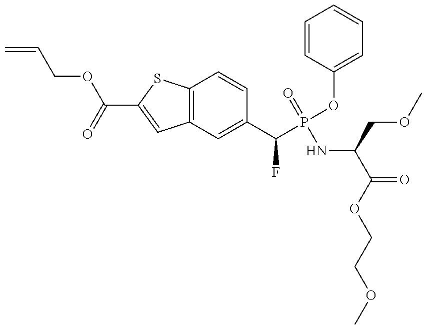 [SMILES: C=CCOC(=O)c1cc2cc([C@H](F)P(=O)(N[C@@H](COC)C(=O)OCCOC)Oc3ccccc3)ccc2s1] | 566 [M + H]⁺ |
| allyl 5-((1R)-fluoro((((S)-1-(2- methoxyethoxy)-1-oxopropan-2- yl)amino)(phenoxy)phosphoryl)meth- yl)benzo[b]thiophene-2- carboxylate | 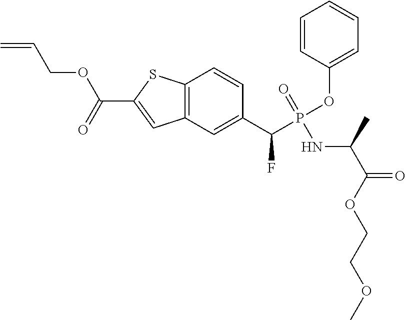 [SMILES: C=CCOC(=O)c1cc2cc([C@H](F)P(=O)(N[C@@H](C)C(=O)OCCOC)Oc3ccccc3)ccc2s1] | 536 [M + H]⁺ |
| allyl 5-((1R)-fluoro((((S)-1-(2- methoxyethoxy)-1-oxopropan-2- yl)amino)(2,2,2- trifluoroethoxy)phosphoryl)methyl) benzo[b]thiophene-2-carboxylate | 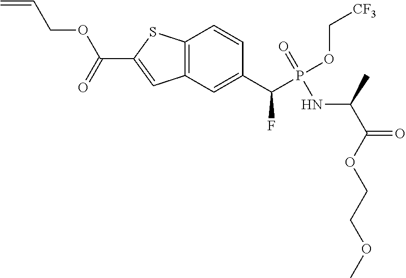 [SMILES: C=CCOC(=O)c1cc2cc([C@H](F)P(=O)(N[C@@H](C)C(=O)OCCOC)OCC(F)(F)F)ccc2s1] | 542 [M + H]⁺ |
| allyl 5-((1R)-fluoro((((S)-1-oxo-1- propoxypropan-2- yl)amino)(phenoxy)phosphoryl)meth- yl)benzo[b]thiophene-2- carboxylate | 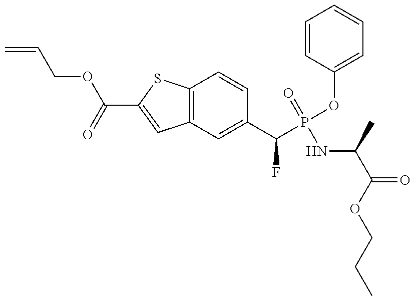 [SMILES: C=CCOC(=O)c1cc2cc([C@H](F)P(=O)(N[C@@H](C)C(=O)OCCC)Oc3ccccc3)ccc2s1] | 520 [M + H]⁺ |
| allyl 5-((1R)-fluoro((((S)-3- methoxy-1-oxo-1-propoxypropan- 2- yl)amino)(phenoxy)phosphoryl)meth- yl)benzo[b]thiophene-2- carboxylate | 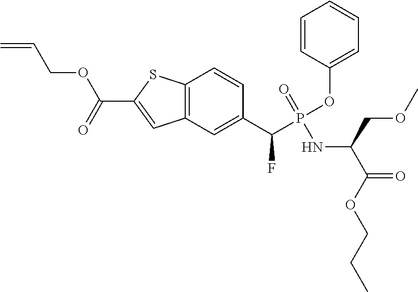 [SMILES: C=CCOC(=O)c1cc2cc([C@H](F)P(=O)(N[C@@H](COC)C(=O)OCCC)Oc3ccccc3)ccc2s1] | 550 [M + H]⁺ |
| allyl 5-((1R)-fluoro((((S)-1-oxo-1- (pyridin-2-ylmethoxy)propan-2- yl)amino)(phenoxy)phosphoryl)meth- yl)benzo[b]thiophene-2- carboxylate | 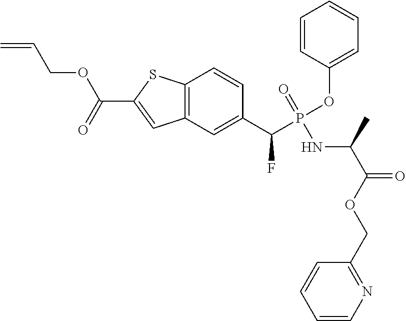 [SMILES: C=CCOC(=O)c1cc2cc([C@H](F)P(=O)(N[C@@H](C)C(=O)OCc3ccccn3)Oc3ccccc3)ccc2s1] | 569 [M + H]⁺ |
| allyl 5-((1R)-fluoro((((S)-1- isobutoxy-1-oxopropan-2- yl)amino)(phenoxy)phosphoryl)meth- yl)benzo[b]thiophene-2- carboxylate | 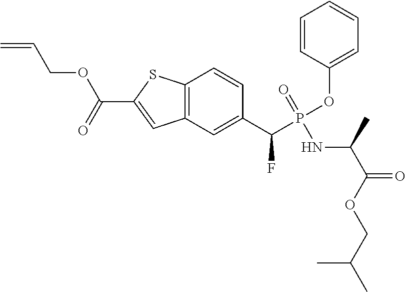 [SMILES: C=CCOC(=O)c1cc2cc([C@H](F)P(=O)(N[C@@H](C)C(=O)OCC(C)C)Oc3ccccc3)ccc2s1] | 534 [M + H]⁺ |
| allyl 5-((1R)-((((S)-1- cyclopropoxy-1-oxopropan-2- yl)amino)(phenoxy)phosphoryl)fluoro- methyl)benzo[b]thiophene-2- carboxylate | 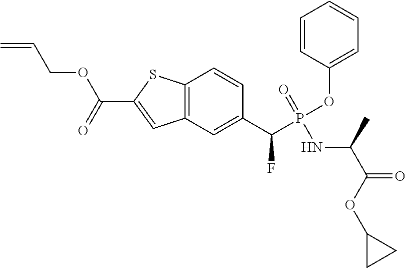 [SMILES: C=CCOC(=O)c1cc2cc([C@H](F)P(=O)(N[C@@H](C)C(=O)OC3CC3)Oc3ccccc3)ccc2s1] | 518 [M + H]⁺ |
| allyl 5-((1R)-((((S)-1-(2- ethylbutoxy)-1-oxopropan-2- yl)amino)(phenoxy)phosphoryl)fluoro- methyl)benzo[b]thiophene-2- carboxylate | 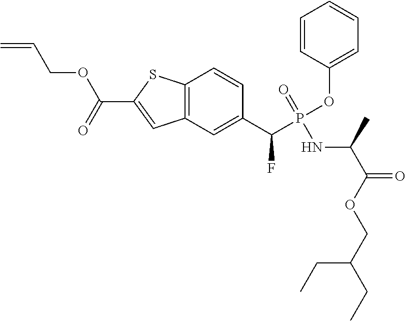 [SMILES: C=CCOC(=O)c1cc2cc([C@H](F)P(=O)(N[C@@H](C)C(=O)OCC(CC)CC)Oc3ccccc3)ccc2s1] | 562 [M + H]⁺ |
| allyl 5-((1R)-((((S)-1-(benzyloxy)- 1-oxopropan-2- yl)amino)(phenoxy)phosphoryl)fluoro- methyl)benzo[b]thiophene-2- carboxylate | 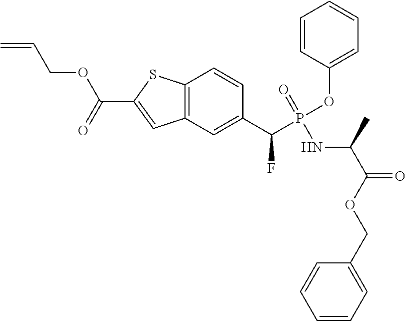 [SMILES: C=CCOC(=O)c1cc2cc([C@H](F)P(=O)(N[C@@H](C)C(=O)OCc3ccccc3)Oc3ccccc3)ccc2s1] | 568 [M + H]⁺ |
| allyl 5-((1R)-fluoro((((S)-1- (neopentyloxy)-1-oxopropan-2- yl)amino)(phenoxy)phoephoryl)meth- yl)benzo[b]thiophene-2- carboxylate | 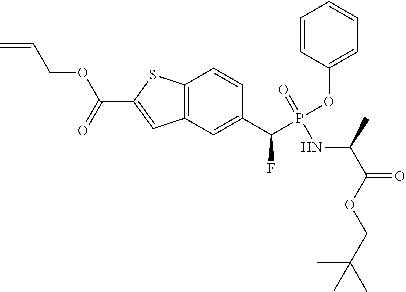 [SMILES: C=CCOC(=O)c1cc2cc([C@H](F)P(=O)(N[C@@H](C)C(=O)OCC(C)(C)C)Oc3ccccc3)ccc2s1] | 548 [M + H]⁺ |
| allyl 5-((1R)-((2,6- dimethylphenoxy)(((S)-1-oxo-1- propoxypropan-2- yl)amino)phosphoryl)fluoromethyl) benzo[b]thiophene-2-carboxylate | 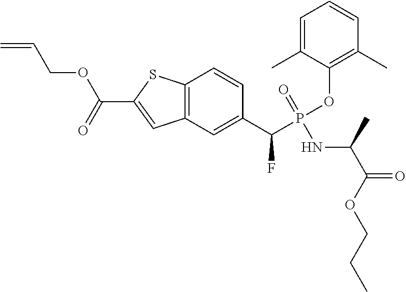 [SMILES: C=CCOC(=O)c1cc2cc([C@H](F)P(=O)(N[C@@H](C)C(=O)OCCC)Oc3c(C)cccc3C)ccc2s1] | 548 [M + H]⁺ |

### Step 4: Preparation of 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylic acid

To a solution of allyl 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylate (90 mg, 156 μmol, 1 eq.) in DCM (5 mL) were added Pd(PPh₃)₄ (9.0 mg, 7.8 μmol, 0.05 eq.) and pyrrolidine (11.0 mg, 156 μmol, 1.0 eq.). The mixture was purged and degassed with N₂ (three times), then stirred at 25° C. for 1 hr. After completion, the mixture was concentrated under reduced pressure. The residue was purified by Biotage® C18 column chromatography to give 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylic acid (50.0 mg, 93.7 μmol, 60%) as a yellow oil. LCMS (ESI): m/z=534 [M+H]⁺.

The intermediates in the table below were prepared using the method described above in Step 4 for the preparation of 5-((1R)-fluoro((((S)-1-((1-methyl-1H-tetrazol-5-yl)methoxy)-1-oxopropan-2-yl)amino)(phenoxy)phosphoryl)methyl)benzo[b]thiophene-2-carboxylic acid and utilizing the appropriate starting materials and modifications.

| Name | Structure | LCMS |
|---|---|---|
| 5-((1R)-fluoro((((S)-1-((4-methyl- 4H-1,2,4-triazol-3-yl)methoxy)-1- oxopropan-2- yl)amino)(phenoxy)phosphoryl)meth- yl)benzo[b]thiophene-2- carboxylic acid | 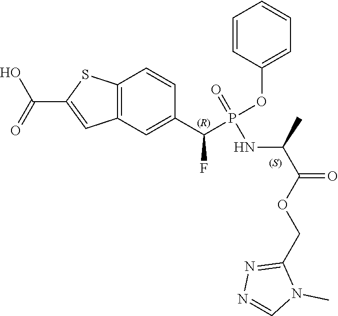 | 533 [M + H]⁺ |
| 5-((1R)-fluoro((((S)-3-methoxy-1- (2-methoxyethoxy)-1-oxopropan-2- yl)amino)(phenoxy)phosphoryl)meth- yl)benzo[b]thiophene-2- carboxylic acid | 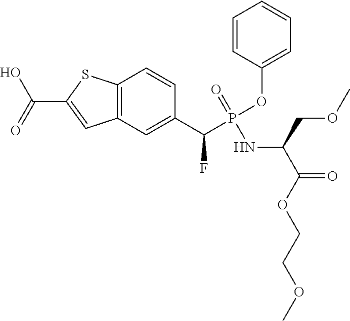 [SMILES: COCCOC(=O)[C@H](COC)NP(=O)(Oc1ccccc1)[C@@H](F)c1ccc2sc(C(=O)O)cc2c1] | 526 [M + H]⁺ |
| 5-((1R)-fluoro((((S)-1-(2- methoxyethoxy)-1-oxopropan-2- yl)amino)(phenoxy)phosphoryl)meth- yl)benzo[b]thiophene-2- carboxylic acid | 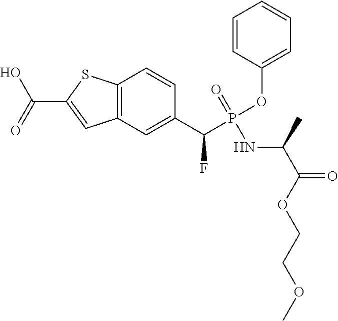 [SMILES: COCCOC(=O)[C@H](C)NP(=O)(Oc1ccccc1)[C@@H](F)c1ccc2sc(C(=O)O)cc2c1] | 496 [M + H]⁺ |
| 5-((1R)-fluoro((((S)-1-(2- methoxyethoxy)-1-oxopropan-2- yl)amino)(2,2,2- trifluoroethoxy)phosphoryl)methyl) benzo[b]thiophene-2-carboxylic acid | 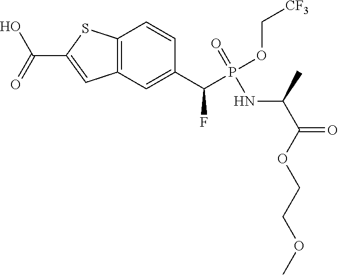 [SMILES: COCCOC(=O)[C@H](C)NP(=O)(OCC(F)(F)F)[C@@H](F)c1ccc2sc(C(=O)O)cc2c1] | 502 [M + H]⁺ |
| 5-((1R)-fluoro((((S)-1-oxo-1- propoxypropan-2- yl)amino)(phenoxy)phosphoryl)meth- yl)benzo[b]thiophene-2- carboxylic acid | 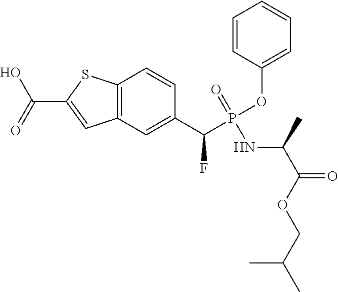 | 480 [M + H]⁺ |
| 5-((1R)-fluoro((((S)-3-methoxy-1- oxo-1-propoxypropan-2- yl)amino)(phenoxy)phosphoryl)meth- yl)benzo[b]thiophene-2- carboxylic acid | 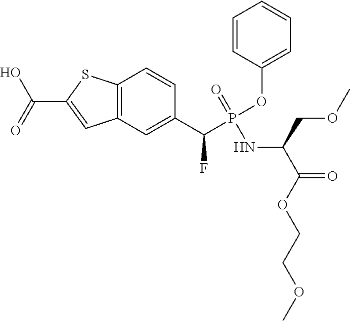 | 510 [M + H]⁺ |
| 5-((1R)-fluoro((((S)-1-oxo-1- (pyridin-2-ylmethoxy)propan-2- yl)amino)(phenoxy)phosphoryl)meth- yl)benzo[b]thiophene-2- carboxylic acid | 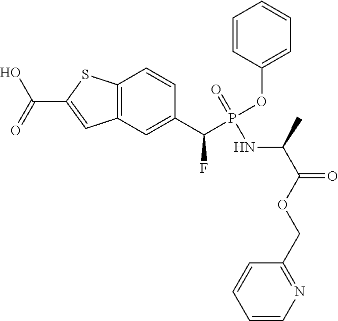 [SMILES: C[C@H](NP(=O)(Oc1ccccc1)[C@@H](F)c1ccc2sc(C(=O)O)cc2c1)C(=O)OCc1ccccn1] | 529 [M + H]⁺ |
| 5-((1R)-fluoro((((S)-1-isobutoxy-1- oxopropan-2- yl)amino)(phenoxy)phosphoryl)meth- yl)benzo[b]thiophene-2- carboxylic acid | 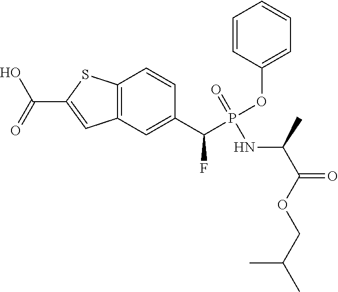 [SMILES: CC(C)COC(=O)[C@H](C)NP(=O)(Oc1ccccc1)[C@@H](F)c1ccc2sc(C(=O)O)cc2c1] | 494 [M + H]⁺ |
| 5-((1R)-((((S)-1-cyclopropoxy-1- oxopropan-2- yl)amino)(phenoxy)phosphoryl)fluoro- methyl)benzo[b]thiophene-2- carboxylic acid | 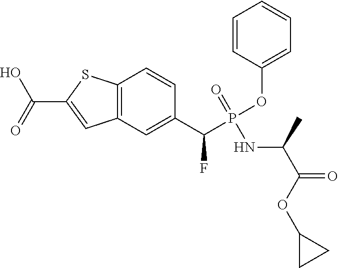 [SMILES: C[C@H](NP(=O)(Oc1ccccc1)[C@@H](F)c1ccc2sc(C(=O)O)cc2c1)C(=O)OC1CC1] | 478 [M + H]⁺ |
| 5-((1R)-((((S)-1-(2-ethylbutoxy)-1- oxopropan-2- yl)amino)(phenoxy)phosphoryl)fluoro- methyl)benzo[b]thiophene-2- carboxylic acid | 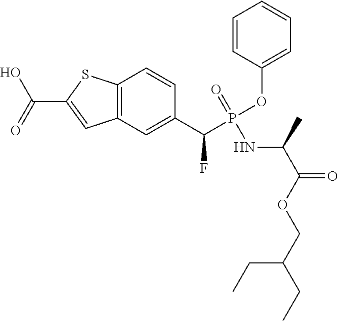 [SMILES: CCC(CC)COC(=O)[C@H](C)NP(=O)(Oc1ccccc1)[C@@H](F)c1ccc2sc(C(=O)O)cc2c1] | 522 [M + H]⁺ |
| 5-((1R)-((((S)-1-(benzyloxy)-1- oxopropan-2- yl)amino)(phenoxy)phosphoryl)fluoro- methyl)benzo[b]thiophene-2- carboxylic acid | 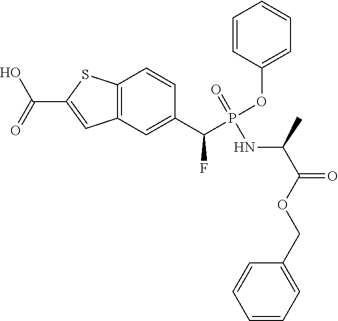 [SMILES: C[C@H](NP(=O)(Oc1ccccc1)[C@@H](F)c1ccc2sc(C(=O)O)cc2c1)C(=O)OCc1ccccc1] | 528 [M + H]⁺. |
| 5-((1R)-((2,6- dimethylphenoxy)(((S)-1-oxo-1- propoxypropan-2- yl)amino)phosphoryl)fluoromethyl) benzo[b]thiophene-2-carboxylic acid | 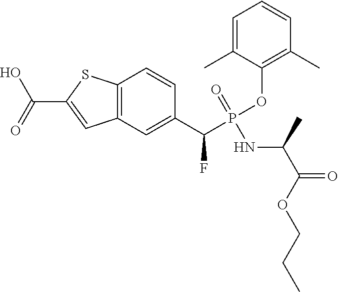 [SMILES: CCCOC(=O)[C@H](C)NP(=O)(Oc1c(C)cccc1C)[C@@H](F)c1ccc2sc(C(=O)O)cc2c1] | 508 [M + H]⁺ |

### Synthesis of 5-((1R)-fluoro((3-(2-methoxyethoxy)phenoxy)(((S)-1-oxo-1-propoxypropan-2-yl)amino)phosphoryl)methyl)benzo[b]thiophene-2-carboxylic acid

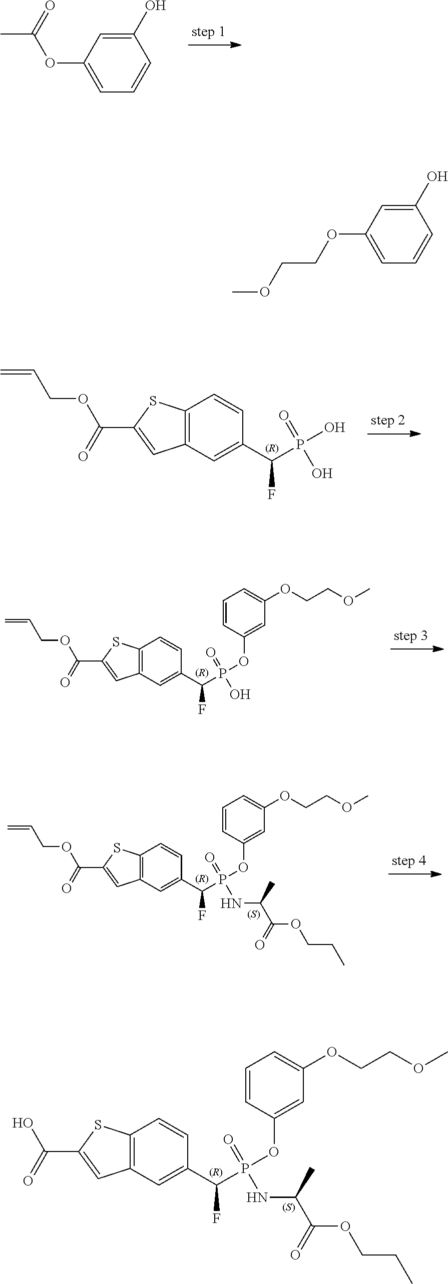

### Step 1: Preparation of 3-(2-methoxyethoxy)phenol

To a solution of 3-hydroxyphenyl acetate (5 g, 32.9 mmol, 1.0 eq.) and K₂CO₃ (13.6 g, 98.7 mmol, 3.0 eq.) in DMF (100 ml) was added 2-methoxyethyl 4-methylbenzenesulfonate (11.4 g, 49.4 mmol, 1.5 eq.). The mixture was stirred at 80° C. for 4 h. After completion, the reaction was cooled to room temperature and 20% NaOH (aq.) was added into the reaction mixture. The resulting mixture was stirred for 0.5 h and then quenched with HCl (1 N, aq.), diluted with H₂O (100 mL) and extracted with DCM (100 mL×3). The organic layers were combined and washed with brine (100 mL), dried over anhydrous Na₂SO₄, and concentrated under reduced pressure. The residue was purified by flash column chromatography on silica gel to afford 3-(2-methoxyethoxy)phenol (4 g, 23.8 mmol, 72%) as a colorless oil. LCMS (ESI): m/z=169 [M+H]⁺.

### Step 2: Preparation of allyl 5-((1R)-fluoro(hydroxy(3-(2-methoxyethoxy)phenoxy)phosphoryl)methyl)benzo[b]thiophene-2-carboxylate

Oxalyl chloride (3.82 g, 30.3 mmol, 5 eq.) was added dropwise to a solution of (R)-((2-((allyloxy)carbonyl)benzo[b]thiophen-5-yl)fluoromethyl)phosphonic acid (3, 2 g, 6.06 mmol, 1 eq.) in dry DCM (30 mL) and DMF (1 drop) at 25° C. The reaction mixture was stirred at 40° C. for an additional 0.5 h. The reaction was monitored by pipetting out a small amount of crude sample and quenching it with MeOH to ensure bis-Cl phosphoryl chloride had been formed completely (bis-methoxy phosphonate was observed by LCMS). After completion, the excess oxalyl chloride and solvent were removed under reduced pressure. The residue was re-dissolved in anhydrous DCM (20 mL), then added to a mixture of 3-(2-methoxyethoxy)phenol (1.07 g, 6.36 mmol, 1.05 eq.) and TEA (6.12 g, 60.6 mmol, 10.0 eq.) in anhydrous DCM (10 mL) at 0° C. The reaction was stirred for 0.5 h. After completion, the reaction mixture was concentrated under reduced pressure. The residue was purified by Biotage® C18 column chromatography to give allyl 5-((1R)-fluoro(hydroxy(3-(2-methoxyethoxy)phenoxy)phosphoryl)methyl)benzo[b]thiophene-2-carboxylate (2.00 g, 4.17 mmol, 69%) as a yellow solid. LCMS (ESI): m/z=481 [M+H]⁺.

### Step 3: Preparation of allyl 5-((1R)-fluoro((3-(2-methoxyethoxy)phenoxy)(((S)-1-oxo-1-propoxypropan-2-yl)amino)phosphoryl)methyl)benzo[b]thiophene-2-carboxylate

Oxalyl chloride (2.63 g, 20.85 mmol, 5 eq.) was added dropwise to the solution of allyl 5-((1R)-fluoro(hydroxy(3-(2-methoxyethoxy)phenoxy)phosphoryl)methyl)benzo[b]thiophene-2-carboxylate (2 g, 4.17 mmol, 1 eq.) in dry DCM (10 mL) and DMF (1 drop) at 25° C. The reaction mixture was stirred at 40° C. for an additional 0.5 h. The reaction was monitored by pipetting out a small amount of crude sample and quenching it with MeOH to ensure mono-C₁ phosphoryl chloride had been formed completely (mono-methoxy phosphonate was observed by LCMS). After completion, the excess oxalyl chloride and solvent were removed under reduced pressure. The residue was re-dissolved in anhydrous DCM (20 mL), then added to a mixture of propyl L-alaninate (1.09 g, 8.34 mmol, 2.0 eq.) and TEA (4.21 g, 41.7 mmol, 10.0 eq.) in anhydrous DCM (20 mL) at 0° C. The reaction was stirred for 0.5 h. After completion, the reaction mixture was concentrated under reduced pressure. The residue was purified by Biotage® C18 column chromatography to give allyl 5-((1R)-fluoro((3-(2-methoxyethoxy)phenoxy)(((S)-1-oxo-1-propoxypropan-2-yl)amino)phosphoryl)methyl)benzo[b]thiophene-2-carboxylate (1.8 g, 3.04 mmol, 73%) as a yellow solid. LCMS (ESI): m/z=594 [M+H]⁺.

### Step 4: 5-((1R)-fluoro((3-(2-methoxyethoxy)phenoxy)(((S)-1-oxo-1-propoxypropan-2-yl)amino)phosphoryl)methyl)benzo[b]thiophene-2-carboxylic acid

To a solution of allyl 5-((1R)-fluoro((3-(2-methoxyethoxy)phenoxy)(((S)-1-oxo-1-propoxypropan-2-yl)amino)phosphoryl)methyl)benzo[b]thiophene-2-carboxylate (900 mg, 1.52 mmol, 1.0 eq) in DCM (6 mL) were added Pd(PPh₃)₄ (175 mg, 152 μmol, 0.1 eq.) and pyrrolidine (109 mg, 1.52 mmol, 1.0 eq.). The mixture was purged and degassed with N₂ three times, then stirred at 25° C. for 1 hr. After completion, the mixture was concentrated under reduced pressure. The residue was purified by Biotage® C18 column chromatography to give 5-((1R)-fluoro((3-(2-methoxyethoxy)phenoxy)(((S)-1-oxo-1-propoxypropan-2-yl)amino)phosphoryl)methyl)benzo[b]thiophene-2-carboxylic acid (370 mg, 0.67 mmol, 44%) as a white solid. LCMS (ESI): m/z=554 [M+H]⁺.

### Synthesis of perfluorophenyl 5-(fluoro((((S)-1-oxo-1-(3,3,3-trifluoropropoxy)propan-2-yl)amino)(phenoxy)phosphoryl)methyl)benzo[b]thiophene-2-carboxylate

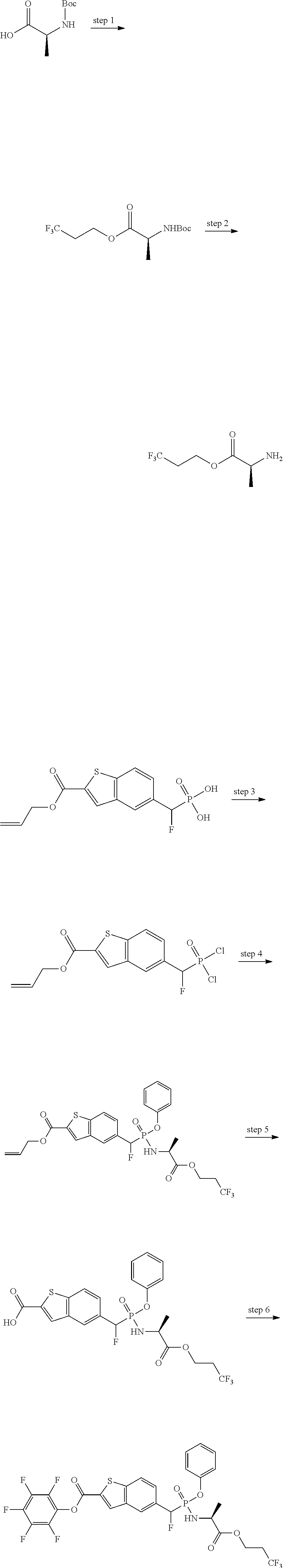

### Step 1: Preparation of (S)-3,3,3-trifluoropropyl 2-((tert-butoxycarbonyl)amino)propanoate

To a solution of (S)-2-((tert-butoxycarbonyl)amino)propanoic acid (5 g, 26.4 mmol, 1 eq) in dimethylformamide (50 mL) was added 3-chloro-1,1,1-trifluoropropane (10 g, 79 mmol, 3 eq) and K₂CO₃ (7.3 g, 53 mmol, 2 eq). The mixture was stirred at 60° C. for 16 h to give a white suspension. The mixture was diluted with water (250 mL) and extracted with EtOAc (1 L×3), the combined organic layers were washed with saturated brine (1 L×3), dried over Na₂SO₄, filtered and concentrated under reduced pressure to give a residue. The residue was purified by flash silica gel chromatography to give a (S)-3,3,3-trifluoropropyl 2-((tert-butoxycarbonyl)amino)propanoate (4.5 g, 15 mmol, 20% yield) as an off-white solid. LCMS (ESI) m/z=286.1 [M+H]⁺.

### Step 2: Preparation of (S)-3,3,3-trifluoropropyl 2-aminopropanoate

The (S)-3,3,3-trifluoropropyl 2-((tert-butoxycarbonyl)amino)propanoate (2 g, 7.0 mmol, 1 eq) was dissolved in DCM (20 mL) and HCL/dioxane (20 mL), the reaction solution was stirred at 25° C. for 1 h. The reaction mixture was concentrated under reduced pressure to give a (S)-3,3,3-trifluoropropyl 2-aminopropanoate (1.1 g, crude) as an off-white solid. ¹H NMR (400 MHz, CD₃OD) δ 4.59-4.40 (m, 2H), 4.15 (q, J=7.2 Hz, 1H), 2.75-2.61 (m, 2H), 1.56 (d, J=7.2 Hz, 3H).

### Step 3: Preparation of allyl 5-((dichlorophosphoryl)fluoromethyl)benzo[b]thiophene-2-carboxylate

To a solution of ((2-((allyloxy)carbonyl)benzo[b]thiophen-5-yl)fluoromethyl)phosphonic acid (0.90 g, 2.7 mmol, 1 eq) in DCM (9 mL) was added N,N-dimethylformamide (20 mg, 0.27 mmol, 0.1 eq) at 25° C., then cooled to 0° C., then oxalic dichloride (1.2 g, 9.5 mmol, 3.5 eq) was added at 0° C., then stirred at 40° C. for 0.5 h. The reaction mixture was concentrated under reduced pressure to give 5-((dichlorophosphoryl)fluoromethyl)benzo[b]thiophene-2-carboxylate (1.0 g, crude) as a yellow oil. LCMS (ESI) m/z=368.0 [M+H]⁺.

### Step 4: Preparation of allyl 5-(fluoro((((S)-1-oxo-1-(3,3,3-trifluoropropoxy)propan-2-yl)amino)(phenoxy)phosphoryl)methyl)benzo[b]thiophene-2-carboxylate

To a solution of allyl 5-((dichlorophosphoryl)fluoromethyl)benzo[b]thiophene-2-carboxylate (1.0 g, 2.7 mmol, 1 eq) in DCM (4 mL), the solution was under N₂ and cooled to 0° C., then phenol (0.20 g, 2.2 mmol, 0.8 eq) in DCM (4 mL) was added in it, ethylbis(propan-2-yl)amine (1.4 g, 11 mmol, 4 eq) in DCM (20 mL) was dropwise added in it and stirred at 0° C. for 5 min, (S)-3,3,3-trifluoropropyl 2-aminopropanoate (0.60 g, 3.3 mmol, 1.2 eq) in DCM (4 mL) was added in it and stirred at 0° C. for 10 min to give a yellow solution. The reaction mixture was concentrated under reduced pressure to give a residue. The residue was purified by flash silica gel chromatography to give a product 5-(fluoro((((S)-1-oxo-1-(3,3,3-trifluoropropoxy)propan-2-yl)amino)(phenoxy)phosphoryl)methyl)benzo[b]thiophene-2-carboxylate (0.70 g, 1.2 mmol, 44% yield) as a white solid. LCMS (ESI) m/z=574.1

### Step 5: Preparation of 5-(fluoro((((S)-1-oxo-1-(3,3,3-trifluoropropoxy)propan-2-yl)amino)(phenoxy)phosphoryl)methyl)benzo[b]thiophene-2-carboxylic acid

To a solution of 5-(fluoro((((S)-1-oxo-1-(3,3,3-trifluoropropoxy)propan-2-yl)amino)(phenoxy)phosphoryl)methyl)benzo[b]thiophene-2-carboxylate (0.58 g, 1.0 mmol, 1 eq) and Pd(PPh₃)₄ (0.17 g, 0.15 mmol, 1.5 eq) in DCM (5 mL) was added pyrrolidine (50 mg, 0.71 mmol, 0.7 eq) at 0° C. The mixture was stirred at 25° C. for 0.5 h to give a yellow suspension. The reaction mixture was filtrated. The filtered and concentrated under reduced pressure to give a residue. The residue was purified by reverse phase (TFA) to lyophilized to give 5-(fluoro((((S)-1-oxo-1-(3,3,3-trifluoropropoxy)propan-2-yl)amino)(phenoxy)phosphoryl)methyl)benzo[b]thiophene-2-carboxylic acid (0.38 g, 0.71 mmol, 70% yield) as a yellow solid. LCMS (ESI) m/z=534.1 [M+H]⁺.

### Step 6: Preparation of perfluorophenyl 5-(fluoro((((S)-1-oxo-1-(3,3,3-trifluoropropoxy)propan-2-yl)amino)(phenoxy)phosphoryl)methyl)benzo[b]thiophene-2-carboxylate

To a solution of 5-(fluoro((((S)-1-oxo-1-(3,3,3-trifluoropropoxy)propan-2-yl)amino)(phenoxy)phosphoryl)methyl)benzo[b]thiophene-2-carboxylic acid (0.40 g, 0.75 mmol, 1 eq) in dimethylformamide (4 mL) was added pyridine (0.18 g, 2.2 mmol, 3 eq), then perfluorophenyl 2,2,2-trifluoroacetate (1.0 g, 3.7 mmol, 5 eq) was added in the mixture at 0° C. to give a yellow solution. The mixture was diluted with water (20 mL) and extracted with EtOAc (20 mL×2), the combined organic layers were washed with saturated brine (20 mL×2), dried over Na₂SO₄, filtered and concentrated under reduced pressure to give a residue. The residue was purified by column chromatography to give a product perfluorophenyl 5-(fluoro((((S)-1-oxo-1-(3,3,3-trifluoropropoxy)propan-2-yl)amino)(phenoxy)phosphoryl)methyl)benzo[b]thiophene-2-carboxylate (0.45 mg, 0.64 mmol, 86% yield) as a white solid. LCMS (ESI) m/z=700.0 [M+H]⁺.

### Syntheses of perfluorophenyl 5-((diethoxyphosphoryl)difluoromethyl)benzo[b]thiophene-2-carboxylate

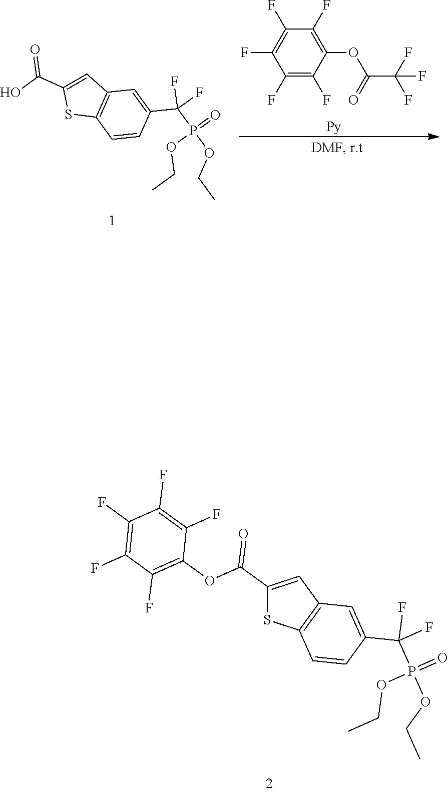

To a solution of 5-((diethoxyphosphoryl)difluoromethyl)benzo[b]thiophene-2-carboxylic acid (1, 3.64 g, 9.99 mmol, 1 eq.) in DMF (30 mL) were added pyridine (1.60 mL, 19.9 mmol, 2 eq.) and 2,3,4,5,6-pentafluorophenyl 2,2,2-trifluoroacetate (2.55 mL, 14.9 mmol, 1.5 eq.) under N₂ with stirring. The resulting mixture was stirred at room temperature for 1 hr. After completion, the reaction mixture was poured into H₂O (20 mL), then extracted with EtOAc (50 mL×3). The organic layers were combined and washed with brine (50 mL), dried over anhydrous Na₂SO₄, and concentrated under reduced pressure to afford perfluorophenyl 5-((diethoxyphosphoryl)difluoromethyl)benzo[b]thiophene-2-carboxylate (2, 5.20 g, quant.) as a white solid. LCMS (ESI) m/z=531.0 [M+H]⁺.

### Preparation of 5-(((benzylamino)(((S)-1-isopropoxy-1-oxopropan-2-yl)amino)phosphoryl)methyl)benzo[b]thiophene-2-carboxylic acid

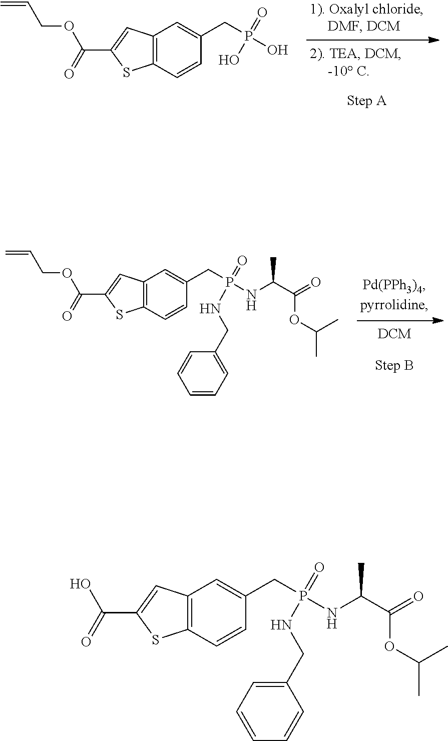 [SMILES: C=CCOC(=O)c1cc2cc(CP(=O)(O)O)ccc2s1 ; C=CCOC(=O)c1cc2cc(CP(=O)(NCc3ccccc3)N[C@@H](C)C(=O)OC(C)C)ccc2s1 ; CC(C)OC(=O)[C@H](C)NP(=O)(Cc1ccc2sc(C(=O)O)cc2c1)NCc1ccccc1]

### Step A: allyl 5-(((benzylamino)(((S)-1-isopropoxy-1-oxopropan-2-yl)amino)phosphoryl)methyl)benzo[b]thiophene-2-carboxylate

Oxalyl chloride (814.4 mg, 6.4 mmol, 10 eq.) was added dropwise to the solution of ((2-((allyloxy)carbonyl)benzo[b]thiophen-5-yl)methyl)phosphonic acid (200 mg, 0.64 mmol, 1.0 eq.) in dry DCM (6 mL) and DMF (1 drop) at 0° C. The reaction was allowed to warm to 40° C., then stirred for additional 1-2 hrs. The reaction was monitored by pipetting out a small amount of crude sample and quenching it with MeOH to ensure bis-Cl phosphoryl chloride allyl 5-((dichlorophosphoryl)methyl)benzo[b]thiophene-2-carboxylate had been formed completely (bis-methoxy phosphonate was observed by LCMS). After completion, the excess of oxalyl chloride and solvent were removed under reduced pressure, and the residue was re-dissolved in anhydrous DCM (5 mL). To the solution was then added BnNH₂ (64.8 mg, 0.64 mmol, 1.0 eq.) in anhydrous DCM (2 mL) and Et₃N (194.32 mg, 1.92 mmol, 3.0 eq.) at −40° C. The reaction was monitored by pipetting out a small amount of crude sample and quenching it with MeOH to ensure the most of the product was allyl 5-(((benzylamino)chlorophosphoryl)methyl)benzo[b]thiophene-2-carboxylate (mono-methoxy phosphonate was observed by LCMS). To the solution was then added isopropyl L-alaninate (107.2 mg, 0.64 mmol, 1.0 eq.) in anhydrous DCM (2 mL) at −40° C. The reaction was allowed to warm to room temperature and stirred for additional 2 hrs. After completion, the reaction was quenched by adding H₂O (10 mL), and extracted with DCM (10 mL×3). The organic layers were combined and washed with brine (20 mL), dried over anhydrous Na₂SO₄, and concentrated under reduced pressure. The residue was purified by flash column chromatography on silica gel to afford allyl 5-(((benzylamino)(((S)-1-isopropoxy-1-oxopropan-2-yl)amino)phosphoryl)methyl)benzo[b]thiophene-2-carboxylate (100 mg, 194.33 μmol, 30%). LCMS (ESI): m/z=515.2 [M+H]⁺.

### Step B: 5-(((benzylamino)(((S)-1-isopropoxy-1-oxopropan-2-yl)amino)phosphoryl)methyl)benzo[b]thiophene-2-carboxylic acid

A solution of allyl 5-(((benzylamino)(((S)-1-isopropoxy-1-oxopropan-2-yl)amino)phosphoryl)methyl)benzo[b]thiophene-2-carboxylate (100 mg, 194 μmol, 1 eq.), Pd(PPh₃)₄ (22.47 mg, 19.4 μmol, 0.1 eq.) and pyrrolidine (13.8 mg, 194 μmol, 1 eq.) in DCM (3 mL) was stirred at room temperature for 1 hr. After completion, the reaction mixture was concentrated under reduced pressure. The residue was purified by flash column chromatography on silica gel to afford 5-(((benzylamino)(((S)-1-isopropoxy-1-oxopropan-2-yl)amino)phosphoryl)methyl)benzo[b]thiophene-2-carboxylic acid (70 mg, 147.5 μmol, 76%). LCMS (ESI): m/z=475.1 [M+H]⁺.

### Synthesis of 5-((bis(((S)-1-isopropoxy-1-oxopropan-2-yl)amino)phosphoryl)methyl)benzo[b]thiophene-2-carboxylic acid

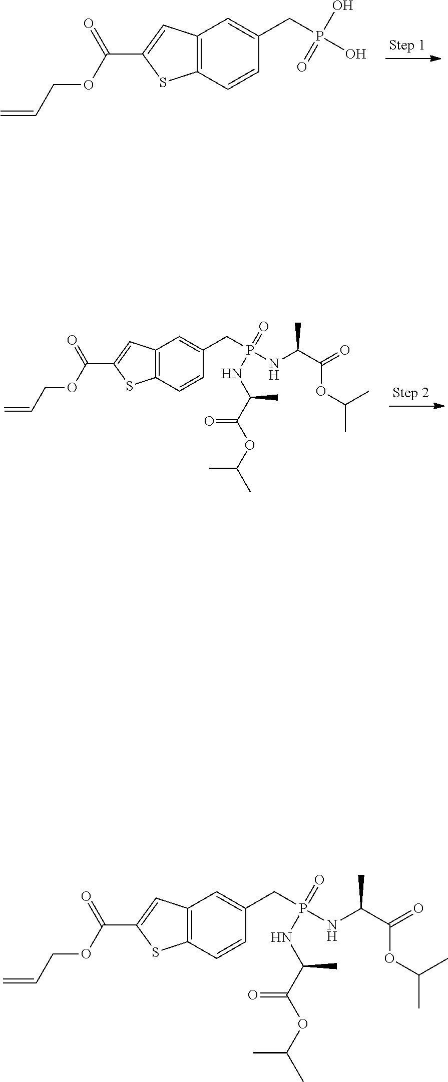

### Step 1: Preparation of diisopropyl 2,2′-((((2-((allyloxy)carbonyl)benzo[b]thiophen-5-yl)methyl)phosphoryl)bis(azanediyl))(2S,2'S)-dipropionate

To a solution of ((2-((allyloxy)carbonyl)benzo[b]thiophen-5-yl)methyl)phosphonic acid (300 mg, 960 μmol, 1.0 eq.) in dry DCM (10 mL) and DMF (cat.) at 0° C. was added oxalyl chloride (609 mg, 4.80 mmol, 5.0 eq.) dropwise. The reaction mixture was allowed to warm to 40° C., then stirred for additional 1-2 h. The reaction was monitored by pipetting out a small amount of crude sample and quenching it with MeOH to ensure bis-Cl phosphoryl chloride had been formed completely (bis-methoxy phosphonate was observed by LCMS). After completion, the excess of oxalyl chloride and solvent were removed under reduced pressure, and the residue was re-dissolved in anhydrous DCM (5 mL). This solution was then added to a mixture of isopropyl L-alaninate (502 mg, 3.83 mmol, 4.0 eq.) and N,N-diisopropylethylamine (620 mg, 4.80 mmol, 5.0 eq.) in anhydrous DCM (10 mL) at 0° C. The reaction was allowed to warm to room temperature and stirred for additional 18 h. The progress was monitored by LCMS. After completion, the reaction was quenched by adding H₂O (10 mL) and extracted with DCM (10 mL×3). The organic layers were combined and washed with brine (20 mL), dried over anhydrous Na₂SO₄, and concentrated under reduced pressure. The residue was purified by flash column chromatography on silica gel to afford diisopropyl 2,2′-((((2-((allyloxy)carbonyl)benzo[b]thiophen-5-yl)methyl)phosphoryl)bis(azanediyl))(2S,2'S)-dipropionate (70.0 mg, 129 μmol, 14% yield). LCMS (ESI): m/z=539.2 [M+H]⁺.

### Step 2: Preparation of 5-((bis(((S)-1-isopropoxy-1-oxopropan-2-yl)amino)phosphoryl)methyl)benzo[b]thiophene-2-carboxylic acid

To a solution of diisopropyl 2,2′-((((2-((allyloxy)carbonyl)benzo[b]thiophen-5-yl)methyl)phosphoryl)bis(azanediyl))(2S,2'S)-dipropionate (70 mg, 129 μmol, 1.0 eq.) in DCM (1 mL) was added pyrrolidine (9.17 mg, 129 μmol, 1.0 eq.) and Pd(PPh₃)₄ (14.9 mg, 12.9 μmol, 0.1 eq.) under N₂, and the resulting mixture was stirred at room temperature for 2 h. After completion, the reaction mixture was cooled down in an ice-bath, then neutralized carefully with HCl (aq. 1M) until the pH was adjusted to pH=4-6. The resulting mixture was extracted with DCM (10 mL×3), and the combined organic layers were washed with brine (10 mL×2), dried over anhydrous Na₂SO₄, then concentrated under reduced pressure. The residue was purified by Biotage® C18 column chromatography to give 5-((bis(((S)-1-isopropoxy-1-oxopropan-2-yl)amino)phosphoryl)methyl)benzo[b]thiophene-2-carboxylic acid (70.0 mg, 140 μmol, 108%, 60% purity) as a colorless oil. LCMS (ESI): m/z=499.2 [M+H]⁺.

### Synthesis of 5-(difluoro(((2-isopropoxy-2-oxoethyl)amino)(phenoxy)phosphoryl)methyl)benzo[b]thiophene-2-carboxylic acid

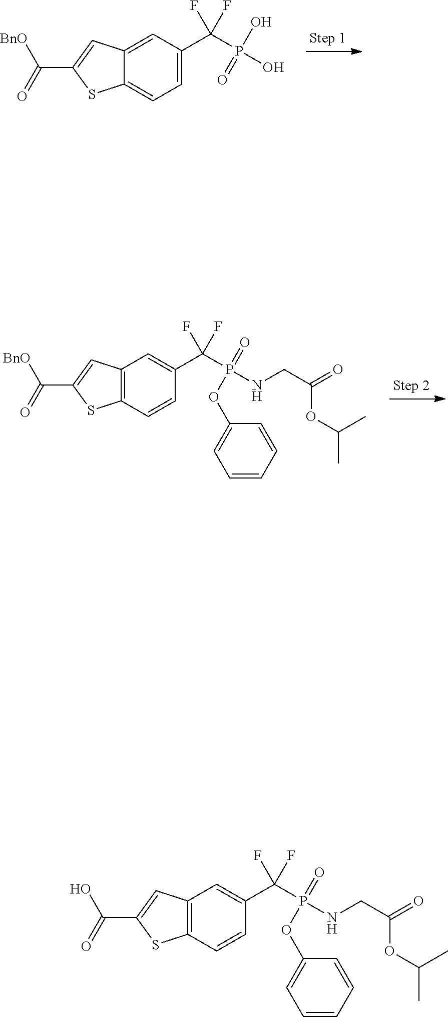 [SMILES: O=C(OCc1ccccc1)c1cc2cc(C(F)(F)P(=O)(O)O)ccc2s1 ; CC(C)OC(=O)CNP(=O)(Oc1ccccc1)C(F)(F)c1ccc2sc(C(=O)OCc3ccccc3)cc2c1 ; CC(C)OC(=O)CNP(=O)(Oc1ccccc1)C(F)(F)c1ccc2sc(C(=O)O)cc2c1]

### Step 1: Preparation of benzyl 5-(difluoro(((2-isopropoxy-2-oxoethyl)amino)(phenoxy)phosphoryl)methyl)benzo[b]thiophene-2-carboxylate

To a solution of ((2-((benzyloxy)carbonyl)benzo[b]thiophen-5-yl)difluoromethyl)phosphonic acid (300 mg, 753 μmol, 1 eq) in methylene chloride (8 mL) at 0° C. was added 2 drops of DMF (cat.) followed by dropwise addition of oxalyl chloride (192 μL, 2.25 mmol, 3 eq). The reaction was warmed up to room temperature and stirred for 2 h. The reaction was not soluble at first but became a clear solution upon adding oxalyl chloride and warming up to room temperature. The reaction was concentrated under reduced pressure. Then, the latter was diluted in methylene chloride (8 mL) and the solution was cooled down to −78° C. A solution of phenol (56.6 mg, 602 μmol, 0.8 eq) and triethylamine (155 μL, 1.12 mmol, 1.5 eq) in DCM (1 mL) was slowly added on the yellow solution over 5 min. The reaction mixture was stirred at −78° C. for 15 min., then warmed up to room temperature and stirred for 2 h. The reaction mixture was cooled down to −78° C. A solution of propan-2-yl 2-aminoacetate (88.2 mg, 753 μmol, 1 eq) and triethylamine (155 μL, 1.12 mmol, 1.5 eq) in DCM (1 mL) was slowly added on the yellow solution over 5 min. The reaction mixture was stirred at −78° C. for 15 min., then warmed up to room temperature and stirred for 18 h. Water (2-3 drops) was added and the reaction was concentrated under reduced pressure. The crude residue was purified by reverse phase chromatography on a 50 g Cis cartridge eluting with 5-100% MeCN in water (with 0.1% formic acid) to give benzyl 5-(difluoro(((2-isopropoxy-2-oxoethyl)amino)(phenoxy)phosphoryl)methyl)benzo[b]thiophene-2-carboxylate (90.0 mg, 156 μmol, 20.8% yield) as a yellow oil. LCMS: m/z=574.2 [M+H]⁺.

### Step 2: Preparation of 5-(difluoro(((2-isopropoxy-2-oxoethyl)amino)(phenoxy)phosphoryl)methyl)benzo[b]thiophene-2-carboxylic acid

To a solution of benzyl 5-(difluoro(((2-isopropoxy-2-oxoethyl)amino)(phenoxy)phosphoryl)methyl)benzo[b]thiophene-2-carboxylate (90 mg, 156 μmol, 1 eq) in anhydrous tetrahydrofuran (10 mL) under nitrogen was added 10% palladium on carbon (50% wet) (166 mg, 78.0 μmol, 0.5 eq). Hydrogen was bubbled for 5 min. and the reaction mixture was stirred at room temperature for 18 h under hydrogen (1 atm). Nitrogen was bubbled int the mixture and it was then filtered over Celite (rinsing with 2-MeTHF) and concentrated under reduced pressure. The isolated product 5-(difluoro(((2-isopropoxy-2-oxoethyl)amino)(phenoxy)phosphoryl)methyl)benzo[b]thiophene-2-carboxylic acid was used without further purification. LCMS: m/z=484.2 [M+H]⁺.

The intermediates in the table below were prepared according to the procedure described for the synthesis of 5-(difluoro(((2-isopropoxy-2-oxoethyl)amino)(phenoxy)phosphoryl)methyl)benzo[b]thiophene-2-carboxylic acid utilizing the appropriate starting materials.

| Name | Structure | LCMS; NMR |
|---|---|---|
| 5-(difluoro((((S)-3- isopropoxy-2- methyl-3- oxopropyl)amino)(phe- noxy)phosphoryl) methyl)benzo[b]thio- phene-2- carboxylic acid | 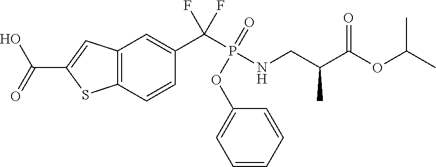 [SMILES: CC(C)OC(=O)[C@@H](C)CNP(=O)(Oc1ccccc1)C(F)(F)c1ccc2sc(C(=O)O)cc2c1] | 512.4 [M + H]⁺ |
| 5-(difluoro((((S)-1- isopropoxy-1-oxo- 3-phenylpropan-2- yl)amino)(phenoxy) phosphoryl)methyl) benzo[b]thiophene- 2-carboxylic acid | 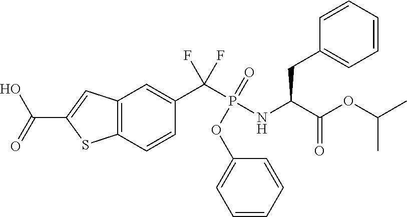 [SMILES: CC(C)OC(=O)[C@H](Cc1ccccc1)NP(=O)(Oc1ccccc1)C(F)(F)c1ccc2sc(C(=O)O)cc2c1] | 574.2 [M + H]⁺ |
| 5-(((((S)-1- (benzyloxy)-1-oxo- 4-phenylbutan-2- yl)amino)(phenoxy) phosphoryl)difluoro- methyl)benzo[b]thi- ophene-2- | 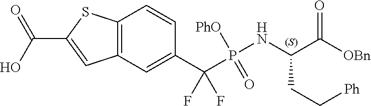 | 636.4 [M+H]+ |
| carboxylic acid | | |
| 5-(((((S)-1- (benzyloxy)-4- methyl-1- oxopentan-2- yl)amino)(phenoxy) phosphoryl)difluoro- methyl)benzo[b]thi- ophene-2- carboxylic acid | 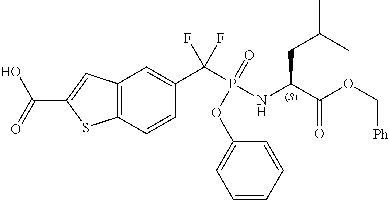 | (400 MHz, DMSO-d₆) δ 13.67 (br. s., 1H), 8.28- 8.13 (m, 3H), 7.73-7.63 (m, 1H), 7.42-7.27 (m, 7H), 7.23-7.06 (m, 3H), 6.79 (td, J = 14.9, 10.6 Hz, 1H), 5.09-4.88 (m, 2H), 3.88-3.64 (m, 1H), 1.49- 1.28 (m, 3H), 0.72-0.54 (m, 6H); 588.2 [M + H]+ |

### Synthesis of perfluorophenyl 5-((1R)-(ethoxy((2-methyl-1-oxo-1-propoxypropan-2-yl)amino)phosphoryl)fluoromethyl)benzo[b]thiophene-2-carboxylate

### Step A: propyl 2-((tert-butoxaycarbonyl)amino)-2-methylpropanoate

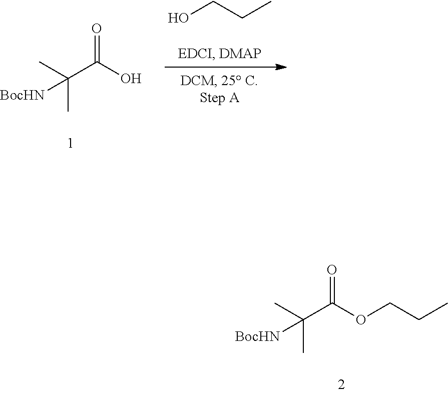

To a solution of 2-((tert-butoxycarbonyl)amino)-2-methylpropanoic acid (1, 2.0 g, 9.8 mmol, 1.0 eq.), EDCI (2.8 g, 14.7 mmol, 1.5 eq.) and DMAP (1.79 g, 14.7 mmol, 1.5 eq.) in DCM (30 mL) was added propan-1-ol (588 mg, 9.8 mmol, 1.0 eq.). The reaction mixture was stirred at 25° C. for 12 hrs. After completion, the mixture was diluted with water (30 mL) and extracted with DCM (30 mL×3), the organic layers were washed with saturated brine (30 mL×2), dried over anhydrous Na₂SO₄ and concentrated under reduced pressure. The residue was purified by flash chromatography on C18 (20%-80% ACN in H₂O) to afford propyl 2-((tert-butoxycarbonyl)amino)-2-methylpropanoate (2, 2.2 g, 9.0 mmol, 92%) as a colorless oil. LCMS (ESI): m/z=246 [M+H]⁺.

### Step B: propyl 2-amino-2-methylpropanoate hydrochloride

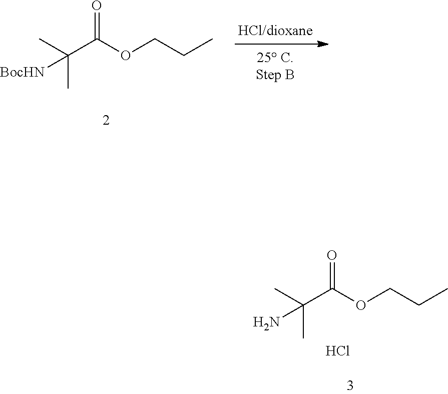

A solution of propyl 2-((tert-butoxycarbonyl) amino)-2-methylpropanoate (2, 2.2 g, 9.0 mmol, 1.0 eq.) in 4M HCl/dioxane (20 mL) was stirred at 25° C. for 2 hrs. After completion, the reaction mixture was concentrated under reduced pressure to give propyl 2-amino-2-methylpropanoate hydrochloride (3, 1.63 g, 9.0 mmol, crude) as a white solid, which was used in the next step directly without further purification. LCMS (ESI): m/z=146 [M+H]⁺.

### Step C: (R)-((2-((allyloxy)carbonyl)benzo[b]thiophen-5-yl)fluoromethyl)phosphonic acid

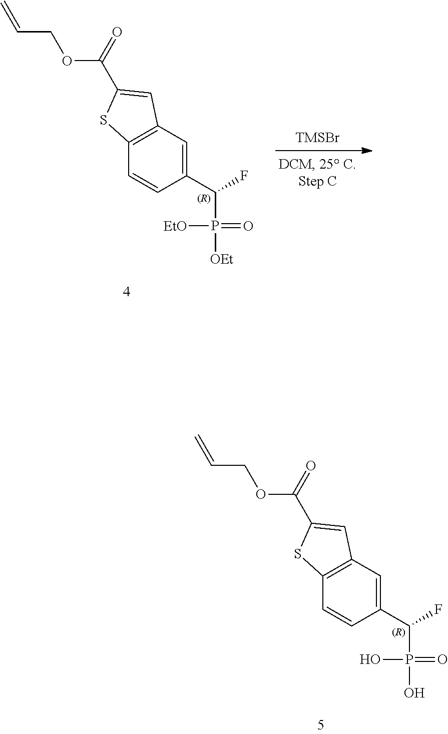

To a solution of methyl allyl (R)-5-((diethoxyphosphoryl)fluoromethyl)benzo[b]thiophene-2-carboxylate (4, 50 g, 130 mmol, 1.0 eq.) in DCM (500 mL) was added TMSBr (100 ml, 766 mol, 5.9 eq.). The resulting mixture was stirred at 25° C. for 16 hrs. After completion, the mixture was concentrated under reduced pressure. The residue was purified by flash chromatography on C18 to give (R)-((2-((allyloxy)carbonyl)benzo[b]thiophen-5-yl)fluoromethyl)phosphonic acid (5, 35 g, 106 mmol, 81%) as a white solid. LCMS (ESI): m/z=331 [M+H]⁺.

### Step D: allyl 5-((1R)-(ethoxy((2-methyl-1-oxo-1-propoxypropan-2-yl)amino)phosphoryl)fluoromethyl)benzo[b]thiophene-2-carboxylate

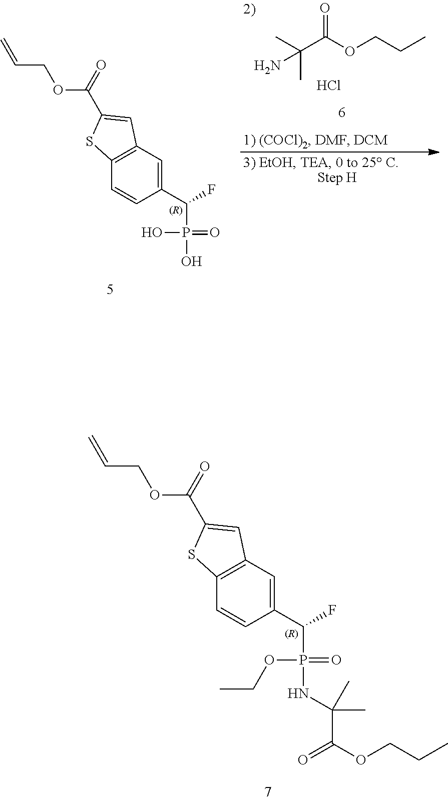

To a solution of allyl 5-((1R)-(ethoxy((2-methyl-1-oxo-1-propoxypropan-2-yl)amino)phosphoryl)fluoromethyl)benzo[b]thiophene-2-carboxylate (5, 5 g, 15.2 mmol, 1.0 eq.) in DCM (60 mL) were added oxalyl chloride (9.6 g, 75.6 mmol, 5.0 eq.) and DMF (0.1 mL) at 25° C. The reaction mixture was stirred at 25° C. for 2 hrs under N₂ atmosphere. The reaction solvent was removed under reduce pressure, the residue was dissolved in DCM (60 mL), triethylamine (7.73 g, 76.5 mmol, 5 eq.) was added to the above mixture at 0° C., then a solution of propyl 2-amino-2-methylpropanoate hydrochloride (6, 3.04 g, 16.7 mmol, 1.1 eq.) in DCM (10 mL) was added to the above mixture dropwise at 0° C. EtOH (6.7 g, 76.5 mmol, 5.0 eq.) was added dropwise after the reaction was stirred at 0° C. for 30 min under N₂ atmosphere. The reaction mixture was stirred at 25° C. for 16 hrs under N₂ atmosphere. After completion, the reaction mixture was poured into water (100 mL) and extracted with DCM (100 mL×2), the combined organic layers were washed with brine (100 mL), dried over anhydrous Na₂SO₄ and concentrated under reduced pressure. The residue was purified by flash chromatography on C18 to give allyl 5-((1R)-(ethoxy((2-methyl-1-oxo-1-propoxypropan-2-yl)amino)phosphoryl)fluoromethyl)benzo[b]thiophene-2-carboxylate (7, 3.0 g, 6.18 mmol, 40%) as a yellow solid. LCMS (ESI): m/z=486 [M+H]⁺.

### Step E: 5-((1R)-(ethoxy((2-methyl-1-oxo-1-propoxypropan-2-yl)amino)phosphoryl)fluoromethyl)benzo[b]thiophene-2-carboxylic acid

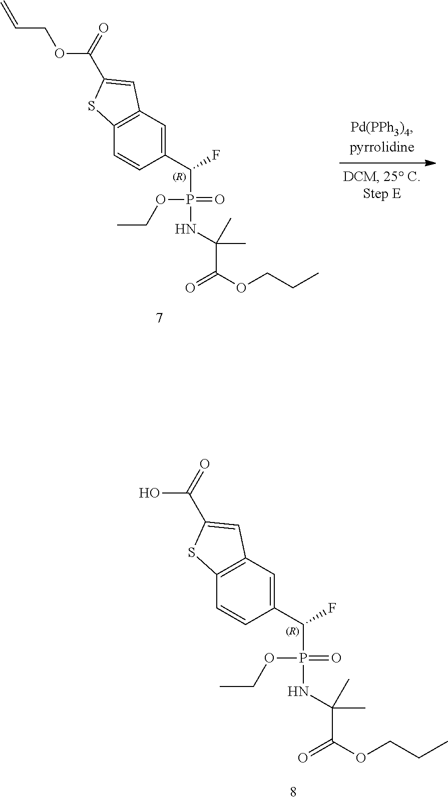

To a solution of allyl 5-((1R)-(ethoxy((2-methyl-1-oxo-1-propoxypropan-2-yl)amino)phosphoryl)fluoromethyl)benzo[b]thiophene-2-carboxylate (7, 3.0 g, 6.18 mmol, 1.0 eq.) and pyrrolidine (439 mg, 6.18 mmol, 1.0 eq.) in DCM (30 mL) was added, Pd(PPh₃)₄ (358 mg, 0.31 mmol, 0.05 eq.). The resulting mixture was stirred at 25° C. for 0.5 hrs under N₂ atmosphere. After completion, the mixture was adjusted to pH=5 with 1N HCl. The reaction mixture was poured into water (30 mL) and extracted with DCM (30 mL×2), the combined organic layers were washed with brine (30 mL), dried over anhydrous Na₂SO₄ and concentrated under reduced pressure to afford 5-((1R)-fluoro((((S)-1-((1s,3R)-3-fluorocyclobutoxy)-1-oxopropan-2-yl)amino)(phenoxy)phosphoryl)methyl)benzo[b]thiophene-2-carboxylic acid (8, 3.0 g, crude) as a yellow solid, which was used in the next step directly without further purification. LCMS (ESI): m/z=446 [M+H]⁺.

### Step F: perfluorophenyl 5-((1R)-(ethoxy((2-methyl-1-oxo-1-propoxypropan-2-yl)amino)phosphoryl)fluoromethyl)benzo[b]thiophene-2-carboxylate

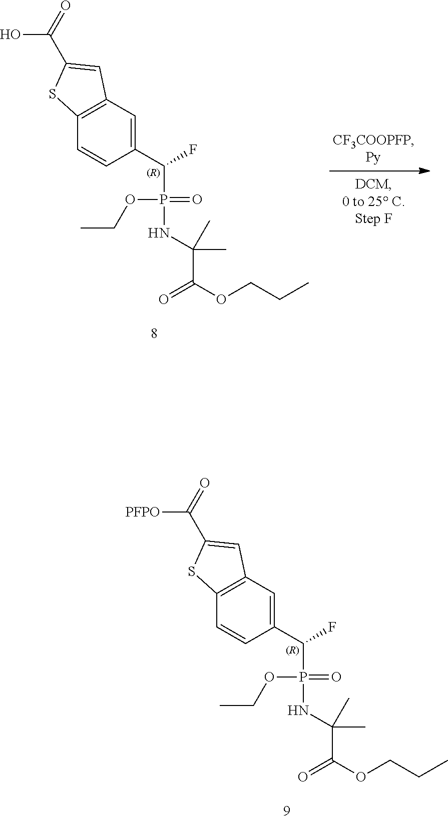

To a solution of 5-((1S)-fluoro((((S)-1-isobutoxy-1-oxopropan-2-yl)amino)(phenoxy)phosphoryl)methyl)benzo[b]thiophene-2-carboxylic acid (8, 3.0 g, crude) in DCM (30 mL) were added pyridine (1.95 g, 24.7 mmol, 4.0 eq.) and CF₃COOPFP (4.34 g, 15.5 mmol, 2.5 eq.) at 0° C. The reaction mixture was warmed up to 25° C. and stirred for 2 hrs. After completion, the reaction mixture was poured into water (30 mL) and extracted with DCM (30 mL×2), the combined organic layers were washed with brine (30 mL), dried over anhydrous Na₂SO₄ and concentrated under reduced pressure. The crude was purified by flash chromatography on C18 (0-90% acetonitrile in H₂O with TFA) to afford perfluorophenyl 5-((1R)-(ethoxy((2-methyl-1-oxo-1-propoxypropan-2-yl)amino)phosphoryl)fluoromethyl)benzo[b]thiophene-2-carboxylate (9, 2.7 g, 4.42 mmol, 71%) as a white solid. ¹H NMR (400 MHz, DMSO-d₆) δ 8.74 (s, 1H), 8.26-8.15 (m, 2H), 7.67 (d, J=8.6 Hz, 1H), 6.05-5.85 (m, 1H), 5.41 (m, 1H), 4.09-3.94 (m, 4H), 1.63-1.51 (m, 2H), 1.33 (m, 3H), 1.26-1.14 (m, 6H), 0.88 (m, 3H). LCMS (ESI): m/z=612 [M+H]⁺.

### Scheme G. General scheme for dimethyl glycine alkoxy benzothiophene esters

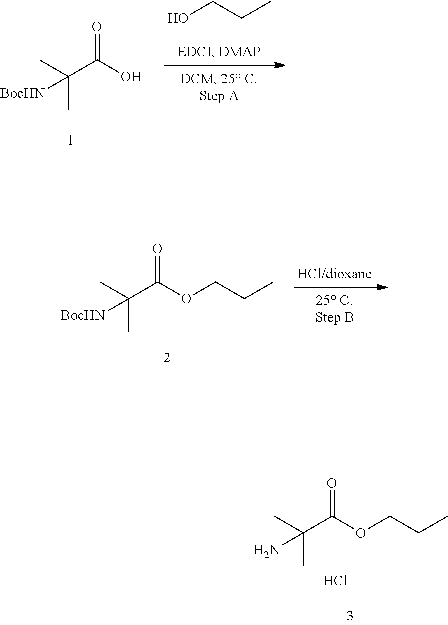

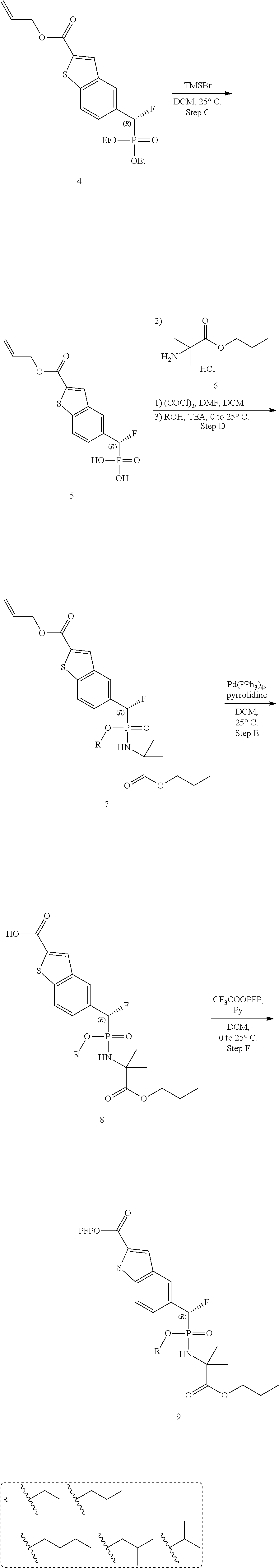

The intermediates in the table below were prepared using a similar protocol outlined above for synthesis of perfluorophenyl 5-((1R)-(ethoxy((2-methyl-1-oxo-1-propoxypropan-2-yl)amino)phosphoryl)fluoromethyl)benzo[b]thiophene-2-carboxylate and utilizing the appropriate intermediate(s) as starting material(s).

| Name | Structure | LC-MS |
|---|---|---|
| propyl 2-((ethoxy((1R)-fluoro(2- (((1-fluoro-2λ¹- diphosphaneyl)oxy)carbonyl)benzo[b] thiophen-5- yl)methyl)phosphoryl)amino)-2- methylpropanoate | 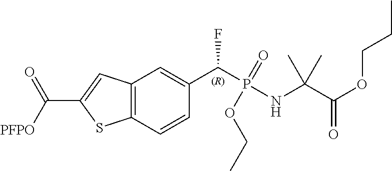 | 612 [M + H]⁺ |
| propyl 2-((((1R)-fluoro(2-(((1- fluoro-2λ¹- diphosphaneyl)oxy)carbonyl)benzo[b] thiophen-5- yl)methyl)(propoxy)phosphoryl)ami- no)-2-methylpropanoate | 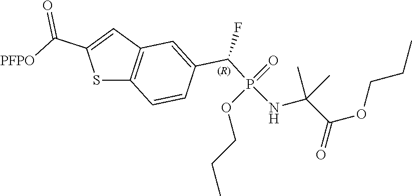 | 626 [M + H]⁺ |
| propyl 2-((butoxy((1R)-fluoro(2- (((1-fluoro-2λ¹- diphosphaneyl)oxy)carbonyl)benzo[b] thiophen-5- yl)methyl)phosphoryl)amino)-2- methylpropanote | 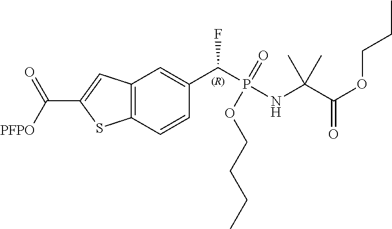 | 640 [M + H]⁺ |
| propyl 2-((((1R)-fluoro(2-(((1- fluoro-2λ¹- diphosphaneyl)oxy)carbonyl)benzo[b] thiophen-5- yl)methyl)(isopropoxy)phosphoryl)a- mino)-2-methylpropanote | 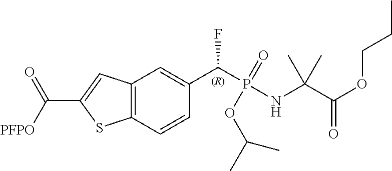 | 626 [M + H]⁺ |
| propyl 2-((((1R)-fluoro(2-(((1- fluoro-2λ¹- diphosphaneyl)oxy)carbonyl)benzo[b] thiophen-5- yl)methyl)(isobutoxy)phosphoryl)a- amino)-2-methylpropanote | 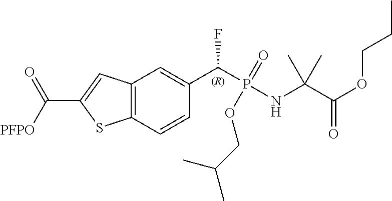 | 640 [M + H]⁺ |

### Synthesis of propyl ((S)-((S)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C17), ((S)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A1), and propyl ((R)-((S)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C25)

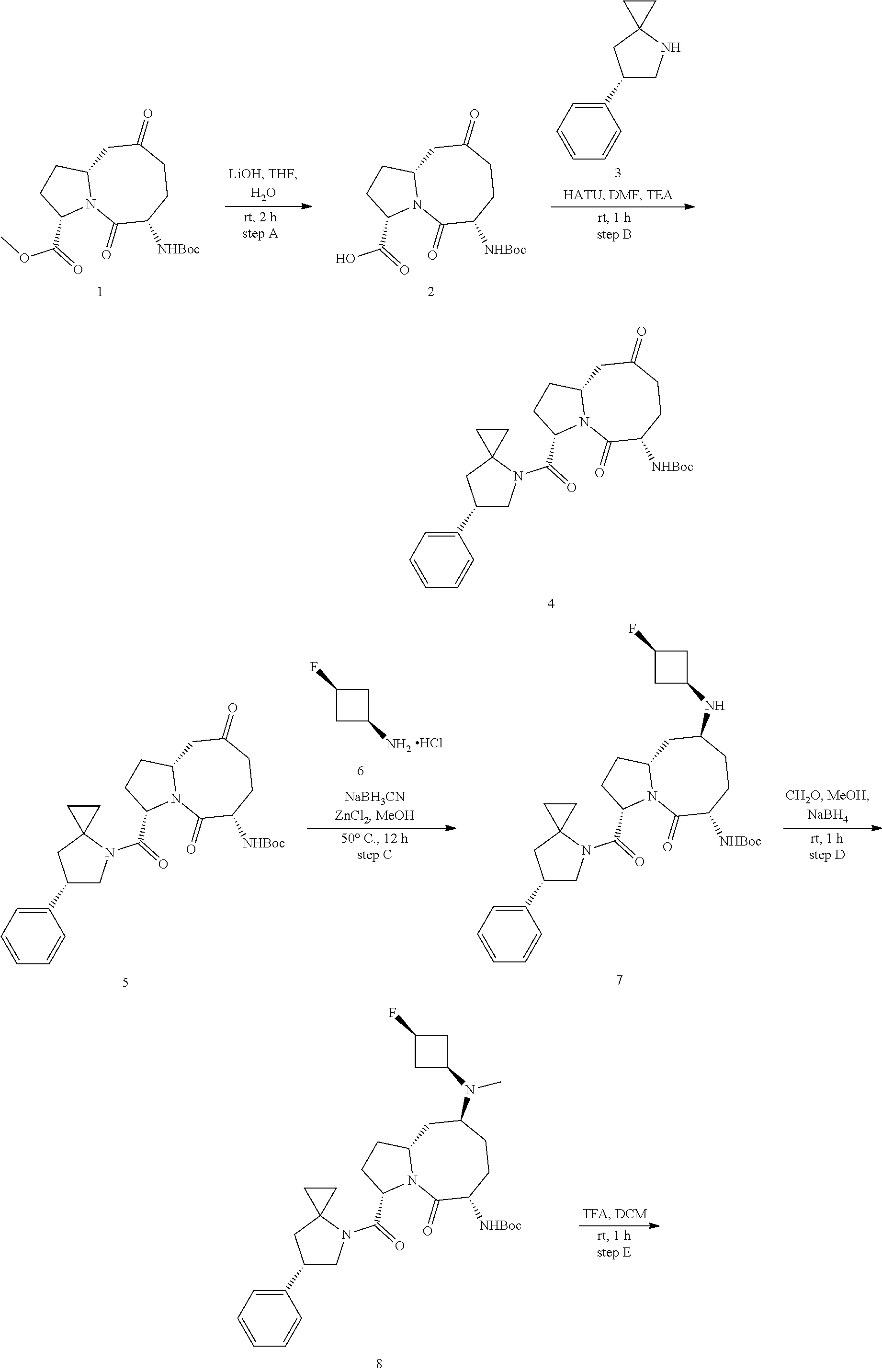

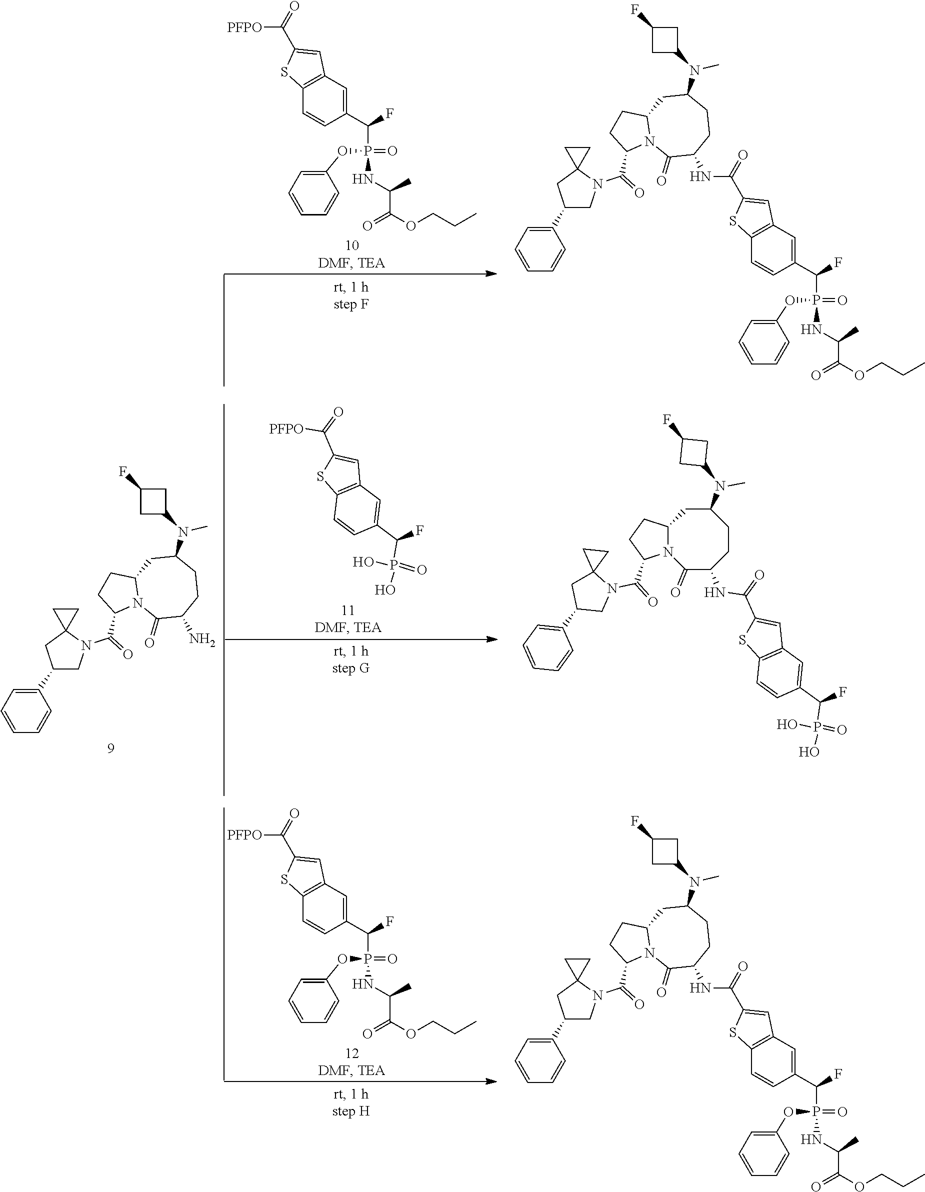

### Step A: (3S,6S,10aR)-6-((tert-butoxycarbonyl)amino)-5,9-dioxodecahydropyrrolo[1,2-a]azocine-3-carboxylic acid

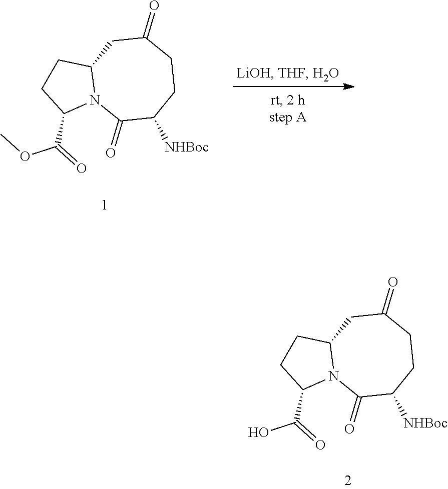 [SMILES: COC(=O)[C@@H]1CC[C@@H]2CC(=O)CC[C@H](NC(=O)OC(C)(C)C)C(=O)N21 ; CC(C)(C)OC(=O)N[C@H]1CCC(=O)C[C@H]2CC[C@@H](C(=O)O)N2C1=O]

To a solution of methyl (3S,6S,10aR)-6-((tert-butoxycarbonyl)amino)-5,9-dioxodecahydropyrrolo[1,2-a]azocine-3-carboxylate (1, 10 g, 28.2 mmol, 1.0 eq.) in THF (50 mL), H₂O (50 mL) was added LiOH—H₂O (3.6 g, 84.7 mmol, 3.0 eq.). The solution was stirred for 2 h at room temperature and the pH was adjusted to pH=3 with 1M HCl (aq.), then extracted with EtOAc (100 mL×3), the organic layers were combined and washed with brine (100 mL), then dried over Na₂SO₄, filtered, then concentrated under vacuum to afford (3S,6S,10aR)-6-((tert-butoxycarbonyl)amino)-5,9-dioxodecahydropyrrolo[1,2-a]azocine-3-carboxylic acid (2, 9.3 g, 27.4 mmol, 97.2% yield) as a white solid. LCMS (ESI): m/z=341 [M+H]⁺.

### Step B: tert-butyl ((3S,6S,10aR)-5,9-dioxo-3-((R)-6-phenyl-4-azaspiro[2.4]heptane-4-carbonyl)decahydropyrrolo[1,2-a]azocin-6-yl)carbamate

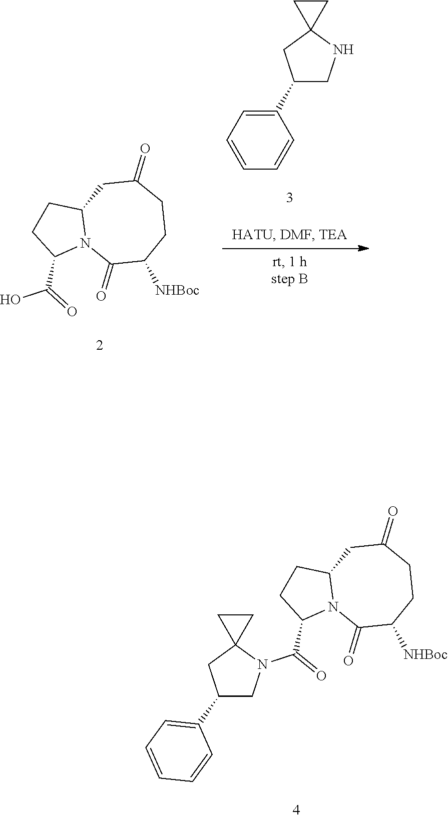 [SMILES: CC(C)(C)OC(=O)N[C@H]1CCC(=O)C[C@H]2CC[C@@H](C(=O)O)N2C1=O ; c1ccc([C@H]2CNC3(CC3)C2)cc1 ; CC(C)(C)OC(=O)N[C@H]1CCC(=O)C[C@H]2CC[C@@H](C(=O)N3C[C@H](c4ccccc4)CC34CC4)N2C1=O]

To a solution of (3S,6S,10aR)-6-((tert-butoxycarbonyl)amino)-5,9-dioxodecahydropyrrolo[1,2-a]azocine-3-carboxylic acid (2, 9 g, 26.5 mmol, 1.0 eq.) in DMF (100 mL) was added HATU (10.1 g, 26.5 mmol, 1 eq.), TEA (8.0 g, 79.0 mmol, 3 eq.), (R)-6-phenyl-4-azaspiro[2.4]heptane (3, 4.6 g, 26.5 mmol, 1 eq). The resulting mixture was stirred for 1 h at room temperature and the solution was poured into H₂O (400 mL), then extracted with EtOAc (100 mL×3), the organic layers were combined and washed with brine (100 mL×3), then dried over Na₂SO₄, then concentrated under vacuum, the crude product was purified by C18 column to afford tert-butyl ((3S,6S,10aR)-5,9-dioxo-3-((R)-6-phenyl-4-azaspiro[2.4]heptane-4-carbonyl)decahydropyrrolo[1,2-a]azocin-6-yl)carbamate (4, 10 g, 20.2 mmol, 77% yield) as a white solid. LCMS (ESI): m/z=496 [M+H]⁺.

### Step C: tert-butyl ((3S,6S,9S,10aR)-9-(((1s,3R)-3-fluorocyclobutyl)amino)-5-oxo-3-((R)-6-phenyl-4-azaspiro[2.4]heptane-4-carbonyl)decahydropyrrolo[1,2-a]azocin-6-yl)carbamate

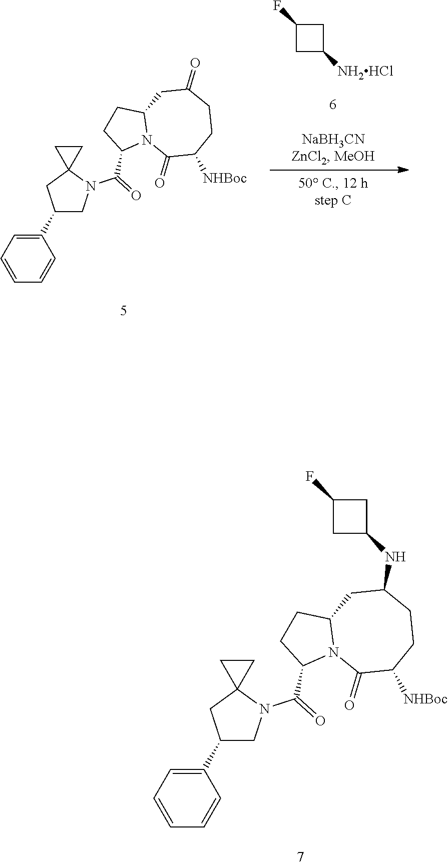

To a solution of tert-butyl ((3S,6S,10aR)-5,9-dioxo-3-((R)-6-phenyl-4-azaspiro[2.4]heptane-4-carbonyl)decahydropyrrolo[1,2-a]azocin-6-yl)carbamate (5, 3 g, 6.06 mmol, 1.0 eq.) in MeOH (60 mL) was added (1s,3s)-3-fluorocyclobutan-1-amine hydrochloride (6, 1.14 g, 9.09 mmol, 1.5 eq.), ZnCl₂ (8.3 g, 60.6 mmol, 10.0 eq.), the solution was stirred for 2 hr at 50° C. under an atmosphere of N₂. Then NaBH₃CN (3.81 g, 60.6 mmol, 10.0 eq.) was added portion wise every 30 mins (2 eq. (762 mg each time) added 5 times), then the resulting mixture was stirred at 50° C. for 12 hr, then cooled to room temperature. The resulting mixture was used to next step without further workup. LCMS (ESI): m/z=569 [M+H]⁺.

### Step D: tert-butyl ((3S,6S,9S,10aR)-9-(((1s,3R)-3-fluorocyclobutyl)(methyl)amino)-5-oxo-3-((R)-6-phenyl-4-azaspiro[2.4]heptane-4-carbonyl)decahydropyrrolo[1,2-a]azocin-6-yl)carbamate

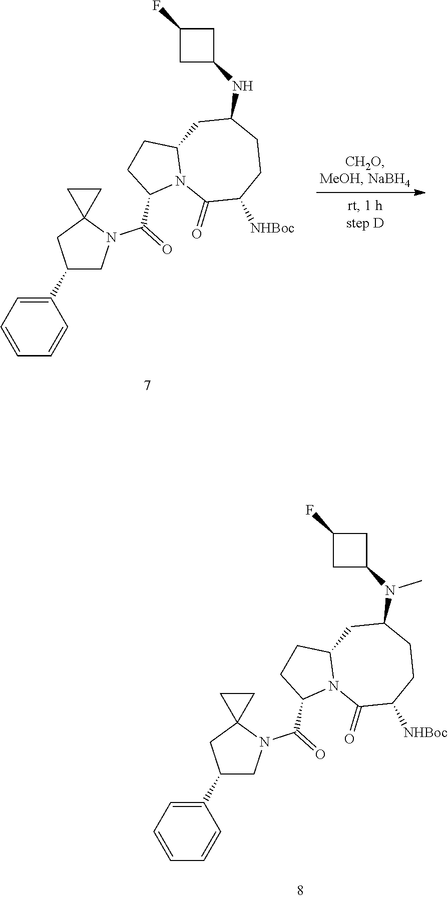 [SMILES: CC(C)(C)OC(=O)N[C@H]1CC[C@H](N[C@H]2C[C@@H](F)C2)C[C@H]2CC[C@@H](C(=O)N3C[C@H](c4ccccc4)CC34CC4)N2C1=O ; CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](F)C1]

To the above mixture (step C) was added 40% formaldehyde aqueous (3 mL) at room temperature, the mixture was stirred for 30 mins, then NaBH₄ (1.15 g, 30.3 mmol, 5 eq.) was added in several portions during 30 mins, the mixture was stirred for additional 30 mins. The reaction was quenched by NH₄Cl (sat. aq.), diluted with H₂O (200 mL) and extracted with EtOAc (100 mL×3). The organic solution was dried over Na₂SO₄, filtered, then concentrated under vacuum. the crude product was purified by C18 column (MeCN:H₂O (0.1% ammonium hydroxide)=5%-70%) to afford tert-butyl ((3S,6S,9S,10aR)-9-(((1s,3R)-3-fluorocyclobutyl)(methyl)amino)-5-oxo-3-((R)-6-phenyl-4-azaspiro[2.4]heptane-4-carbonyl)decahydropyrrolo[1,2-a]azocin-6-yl)carbamate (8, 2.8 g, 4.81 mmol, 79.4% yield two steps) as a white solid. LCMS (ESI): m/z=583 [M+H]⁺.

### Step E: (3S,6S,9S,10aR)-6-amino-9-(((1s,3R)-3-fluorocyclobutyl)(methyl)amino)-3-((R)-6-phenyl-4-azaspiro[2.4]heptane-4-carbonyl)octahydropyrrolo[1,2-a]azocin-5(1H)-one

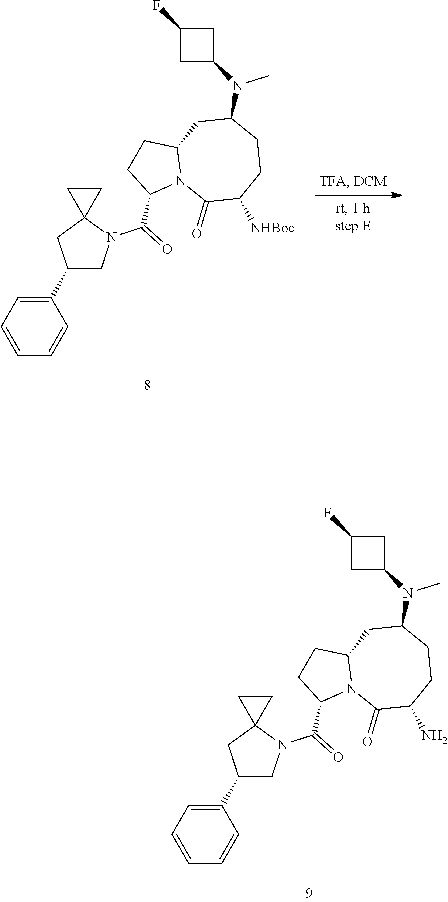 [SMILES: CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](F)C1 ; CN([C@H]1CC[C@H](N)C(=O)N2[C@H](CC[C@H]2C(=O)N2C[C@H](c3ccccc3)CC23CC3)C1)[C@H]1C[C@@H](F)C1]

To a solution of tert-butyl ((3S,6S,9S,10aR)-9-(((1s,3R)-3-fluorocyclobutyl)(methyl)amino)-5-oxo-3-((R)-6-phenyl-4-azaspiro[2.4]heptane-4-carbonyl)decahydropyrrolo[1,2-a]azocin-6-yl)carbamate (8, 2.8 g, 4.81 mmol, 1.0 eq.) in DCM (30 mL) was added TFA (15 mL). The solution was stirred for 1 hr. at room temperature. The resulting mixture was concentrated to dryness under reduced pressure to afford crude product (3S,6S,9S,10aR)-6-amino-9-(((1s,3R)-3-fluorocyclobutyl)(methyl)amino)-3-((R)-6-phenyl-4-azaspiro[2.4]heptane-4-carbonyl)octahydropyrrolo[1,2-a]azocin-5(1H)-one (9, 2.8 g, TFA salt) as a yellow oil. LCMS (ESI): m/z=483 [M+H]⁺.

### Step F: propyl ((S)-((S)-fluoro(2-(((3S,6S,9S,10aR)-9-(((1s,3R)-3-fluorocyclobutyl)(methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

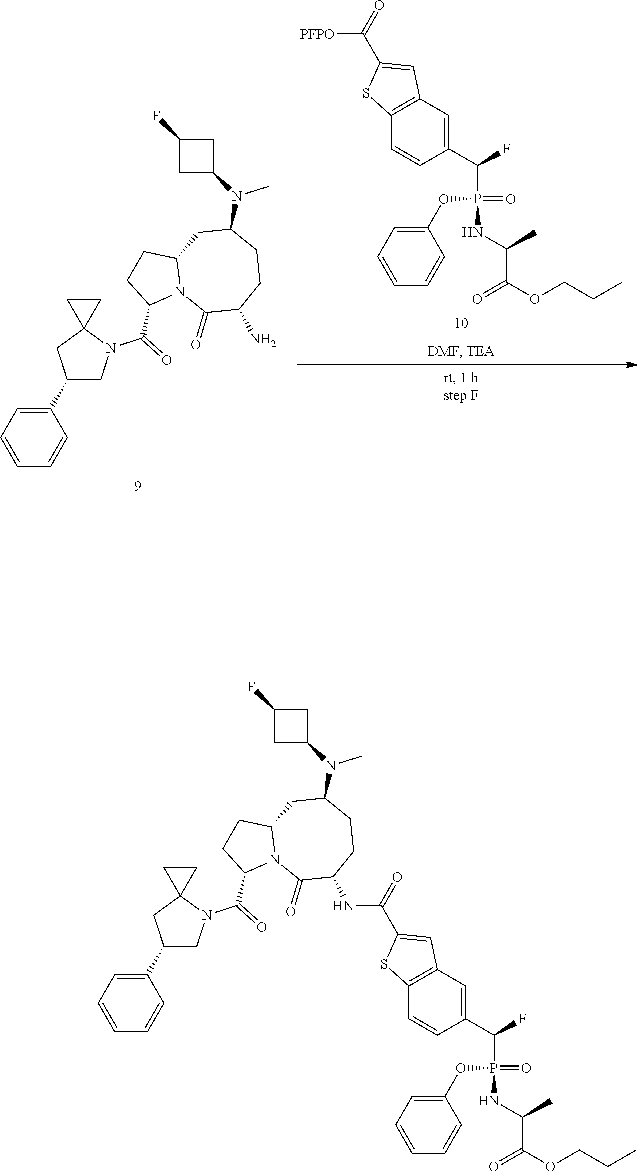

To a solution of (3S,6S,9S,10aR)-6-amino-9-(((1s,3R)-3-fluorocyclobutyl)(methyl)amino)-3-((R)-6-phenyl-4-azaspiro[2.4]heptane-4-carbonyl)octahydropyrrolo[1,2-a]azocin-5(1H)-one (9, 954 mg, 1.70 mmol, 1 eq) in DMF (10 mL) was added TEA (515 mg, 5.10 mmol, 3 eq.), propyl ((S)-((1S)-fluoro(2-(((1-fluoro-2l1-diphosphanyl)oxy)carbonyl)benzo[b]thiophen-5-yl)methyl)(phenoxy)phosphoryl)-L-alaninate (10, 825 mg, 1.70 mmol, 1 eq.). The solution was stirred for 1 hr at room temperature. The crude product was purified by C18 column chromatography (MeCN:H₂O (0.1% NH₄CO₃)=5%-80%) to afford propyl ((S)-((S)-fluoro(2-(((3S,6S,9S,10aR)-9-(((1s,3R)-3-fluorocyclobutyl)(methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C17, 1.09 g, 1.16 mmol, 68.1%) as white solid. LCMS (ESI): m/z=944.6 [M+H]⁺. ¹H NMR (400 MHz, DMSO-d₆) δ (ppm) 10.21-9.42 (m, 1H), 8.94-8.74 (m, 1H), 8.30-8.24 (m, 1H), 8.12-8.04 (m, 2H), 7.65-7.58 (m, 1H), 7.40-7.28 (m, 6H), 7.25-7.16 (m, 4H), 6.24-6.08 (m, 2H), 5.01-4.78 (m, 2H), 4.60-4.48 (m, 2H), 4.19-4.10 (m, 1H), 3.94-3.88 (m, 2H), 3.80-3.67 (m, 3H), 3.59-3.50 (m, 2H), 3.42-3.04 (m, 1H), 2.83-2.62 (m, 2H), 2.61-2.55 (m, 3H), 2.48-2.35 (m, 2H), 2.26-1.47 (m, 15H), 0.95 (d, J=7.1 Hz, 3H), 0.84-0.78 (m, 3H), 0.62-0.44 (m, 2H). ¹⁹F NMR (377 MHz, DMSO-d₆) δ (ppm) −167.05 (s), −193.13-−195.64 (m). ³¹P NMR (162 MHz, DMSO-d₆) δ (ppm) 16.93 (d, J=82.4 Hz).

### Step G: ((S)-fluoro(2-(((3S,6S,9S,10aR)-9-(((1s,3R)-3-fluorocyclobutyl)(methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

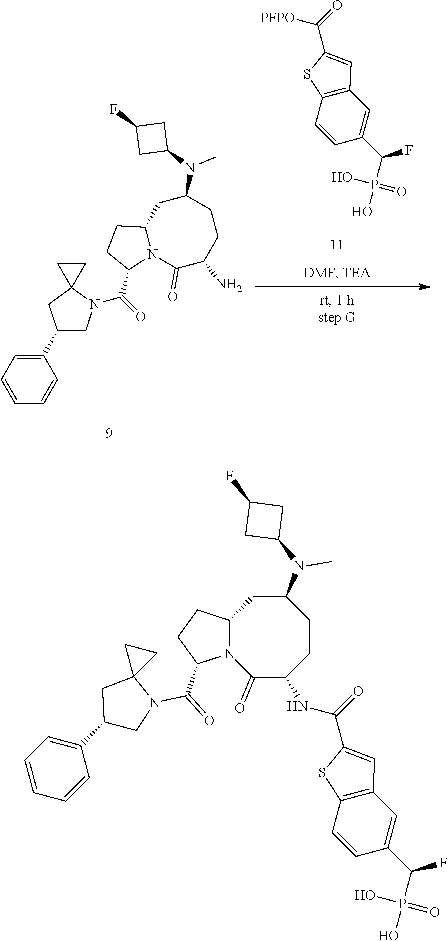

To a solution of (3S,6S,9S,10aR)-6-amino-9-{methyl[(1s,3s)-3-fluorocyclobutyl]amino}-3-[(6R)-6-phenyl-4-azaspiro[2.4]heptane-4-carbonyl]-decahydropyrrolo[1,2-a]azocin-5-one (9, 40 mg, 82.8 μmol, 1 eq.) in DMF (3 mL) was added N,N-diisopropylethylamine (53.5 mg, 414 μmol, 5 eq.) and [(S)-fluoro({2-[(2,3,4,5,6-pentafluorophenoxy)carbonyl]-1-benzothiophen-5-yl})methyl]phosphonic acid (11, 37.7 mg, 82.8 μmol, 1 eq.) 25° C. The reaction mixture was stirred at 25° C. for 1 hr. The crude product was purified by prep-HPLC (C18, 0-50% acetonitrile in H₂O with TFA) to afford [(S)-(2-{[(3S,6S,9S,10aR)-9-{methyl[(1s,3s)-3-fluorocyclobutyl]amino}-5-oxo-3-[(6R)-6-phenyl-4-azaspiro[2.4]heptane4-carbonyl]-decahydropyrrolo[1,2-a]azocin-6-yl]carbamoyl}-1-benzothiophen-5-yl)(fluoro)methyl]phosphonic acid (Compound A1, 30.0 mg, 39.7 μmol, 48.0%) as a white solid. LCMS (ESI): m/z=755.3 [M+H]⁺. ¹H NMR (400 MHz, DMSO-d6) δ (ppm) 10.26-9.53 (m, 1H), 8.91-8.72 (m, 1H), 8.25 (s, 1H), 8.05-7.95 (m, 2H), 7.53 (d, J=8.5 Hz, 1H), 7.38-7.29 (m, 4H), 7.25-7.18 (m, 1H), 5.82 (dd, J=44.4, 7.9 Hz, 1H), 4.99-4.81 (m, 2H), 4.61-4.49 (m, 2H), 4.19-4.11 (m, 1H), 3.86-3.68 (m, 2H), 3.65-3.48 (m, 2H), 3.41-3.05 (m, 1H), 2.79-2.62 (m, 2H), 2.57 (s, 3H), 2.48-2.34 (m, 2H), 2.31-1.50 (m, 13H), 0.63-0.41 (m, 2H). ¹⁹F NMR (377 MHz, DMSO-d₆) δ (ppm) −166.57-−167.90 (m), −193.77-−195.72 (m). ³¹P NMR (162 MHz, DMSO-d₆) δ (ppm) 10.91 (d, J=81.7 Hz), 6 (ppm) 10.81 (d, J=79.8 Hz).

### Step H: propyl ((R)-((S)-fluoro(2-(((3S,6S,9S,10aR)-9-(((1s,3R)-3-fluorocyclobutyl)(methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

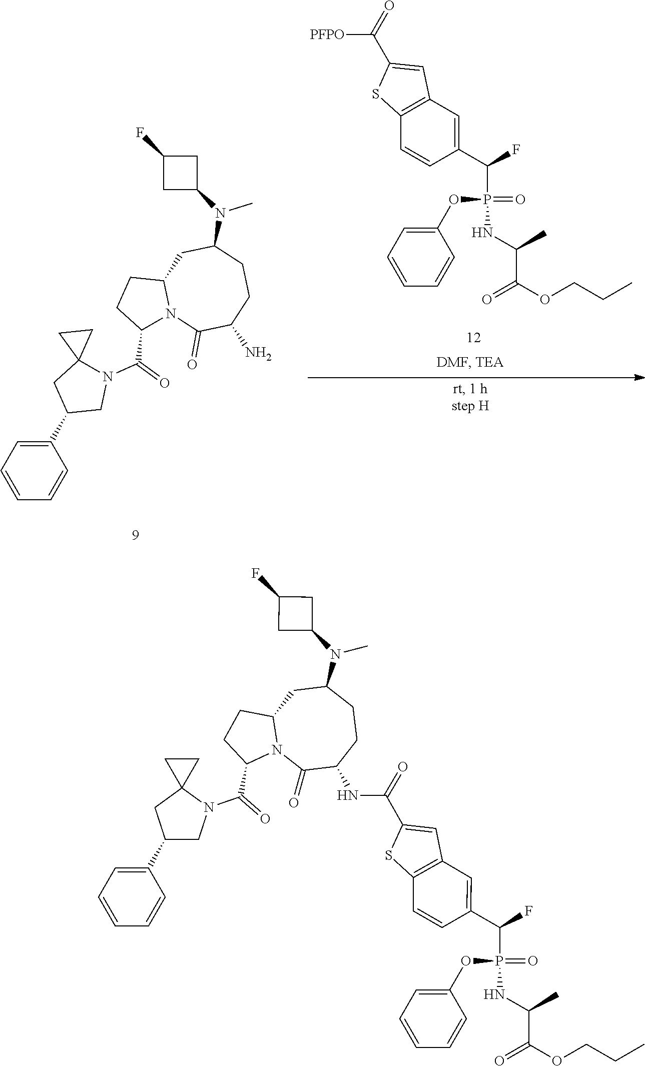

To a solution of (3S,6S,9S,10aR)-6-amino-9-(((1s,3R)-3-fluorocyclobutyl)(methyl)amino)-3-((R)-6-phenyl-4-azaspiro[2.4]heptane-4-carbonyl)octahydropyrrolo[1,2-a]azocin-5(1H)-one (9, 954 mg, 1.70 mmol, 1 eq.) in DMF (10 mL) was added TEA (515 mg, 5.10 mmol, 3 eq.), perfluorophenyl-5-((S)-fluoro((R)-(((S)-1-oxo-1-propoxypropan-2-yl)amino)(phenoxy)phosphoryl)methyl)benzo[b]thiophene-2-carboxylate (12, 825 mg, 1.70 mmol, 1 eq.). The solution was stirred for 1 hr at room temperature. The crude product was purified by C18 column chromatography (MeCN:H₂O (0.1% TFA)=5%-50%) to afford propyl ((R)-((S)-fluoro(2-(((3S,6S,9S,10aR)-9-(((1s,3R)-3-fluorocyclobutyl)(methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C25, 724 mg, 0.767 mmol, 60.3%) as white solid. LCMS (ESI): m/z=944.6 [M+H]⁺. ¹H NMR (400 MHz, DMSO-d₆) δ (ppm) 10.05-9.43 (m, 1H), 8.94-8.75 (m, 1H), 8.32-8.21 (m, 1H), 8.11-7.98 (m, 2H), 7.60 (d, J=8.7 Hz, 1H), 7.39-7.25 (m, 6H), 7.24-7.09 (m, 4H), 6.32-6.14 (m, 2H), 5.04-4.75 (m, 2H), 4.64-4.44 (m, 2H), 4.22-4.06 (m, 1H), 3.97-3.82 (m, 3H), 3.79-3.69 (m, 2H), 3.56-3.53 (m, 2H), 3.36-2.99 (m, 1H), 2.84-2.62 (m, 2H), 2.61-2.55 (m, 3H), 2.48-2.31 (m, 2H), 2.26-1.43 (m, 15H), 1.17 (d, J=7.0 Hz, 3H), 0.81 (t, J=7.4 Hz, 3H), 0.63-0.44 (m, 2H). (TFA salt). ¹⁹F NMR (376 MHz, DMSO) δ (ppm) −166.86-−167.58 (m), −195.89-−196.42 (m). ³¹P NMR (162 MHz, DMSO-d6) δ (ppm) 17.34 (d, J=86.8 Hz).

### Synthesis of propyl (((R)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(2,2,2-trifluoroethoxy)phosphoryl)-L-alaninate (Compound C64)

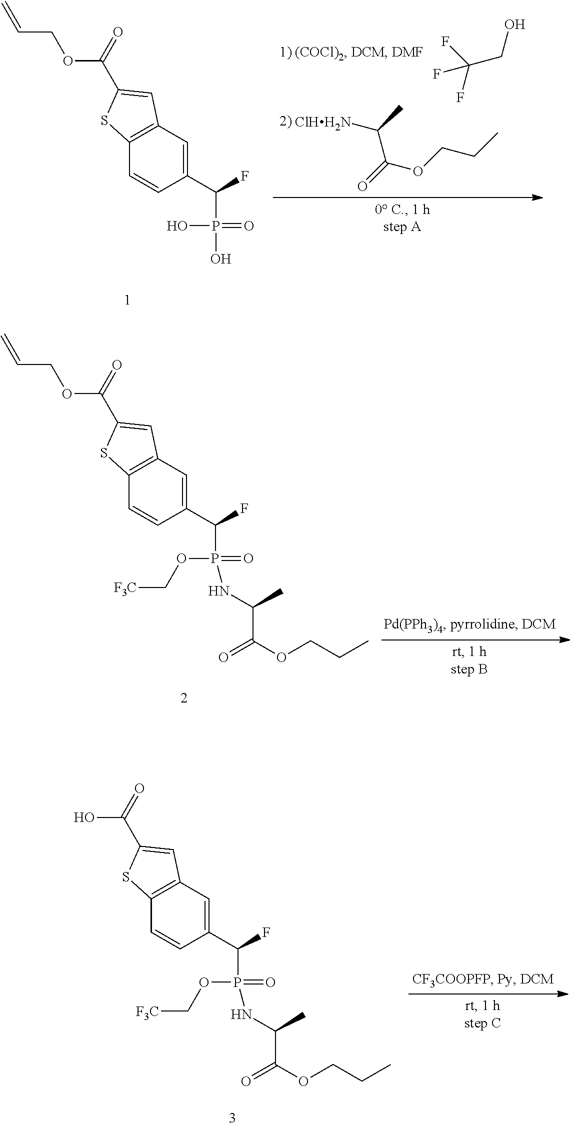

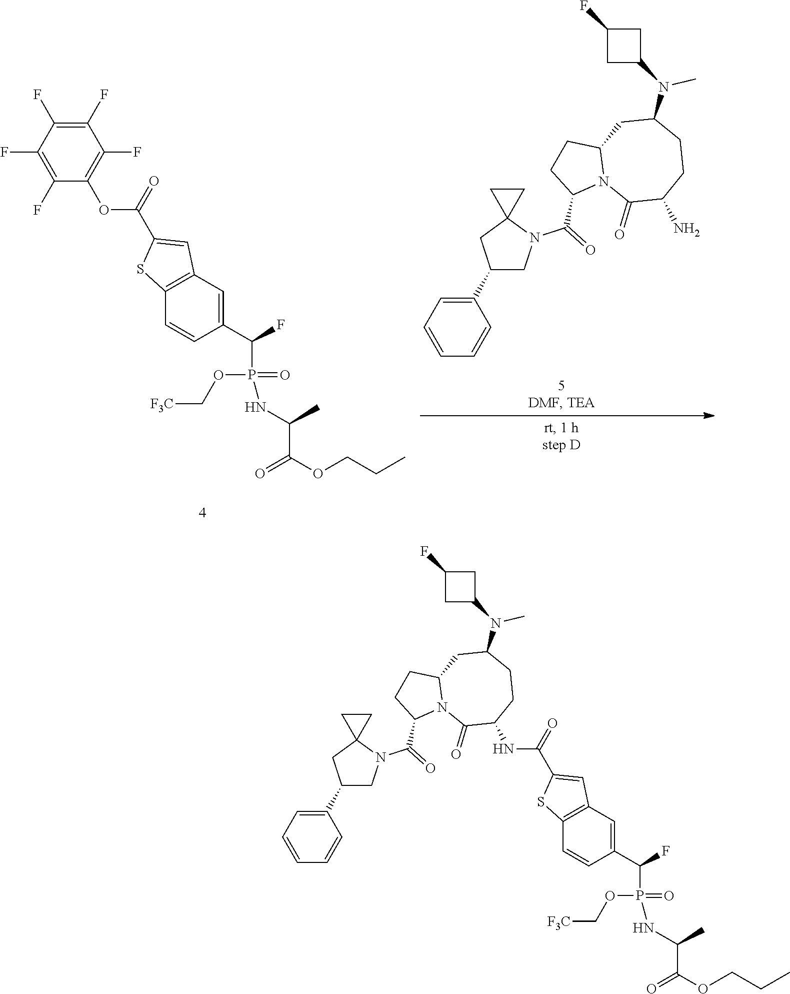

### Step A: allyl 5-((1S)-fluoro((((S)-1-oxo-1-propoxypropan-2-yl)amino)(2,2,2-trifluoroethoxy)phosphoryl)methyl)benzo[b]thiophene-2-carboxylate

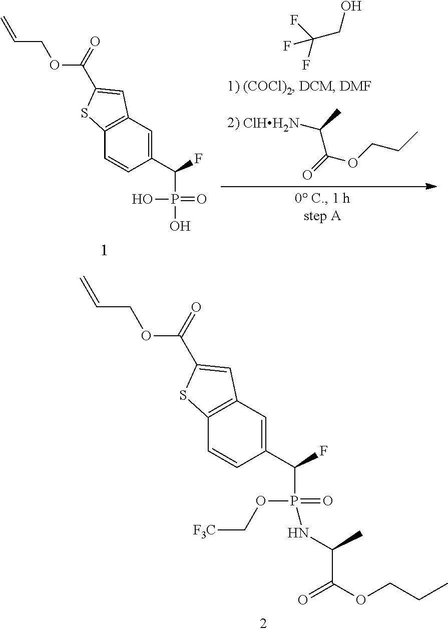

To a solution of (S)-((2-((allyloxy)carbonyl)benzo[b]thiophen-5-yl)fluoromethyl)phosphonic acid (1, 1.3 g, 3.94 mmol, 1 eq.) in DCM (20 mL) was added DMF (catalytic amount), and oxalyl chloride (2.5 g, 19.7 mmol, 5 eq.). The resulting mixture was stirred for 1 h at room temperature, then DCM was removed under reduced pressure. The residue was redissolved in DCM (20 mL) and cooled to 0° C., and 2,2,2-trifluoroethan-1-ol (394 mg, 3.94 mmol, 1 eq.) was added to the solution, TEA (2 g, 19.7 mmol, 5 eq.) was added dropwise to the solution, the mixture was stirred for 1 h at 0° C. Propyl L-alaninate hydrochloride (1.32 g, 7.88 mmol, 2 eq.) was added to the mixture, then stirred for 10 mins, then H₂O (50 mL) was added to the solution, and extracted with DCM (50 mL×2), the organic layers were combined and washed with brine (20 mL×3), then dried over Na₂SO₄, filtered, and concentrated to dryness under reduced pressure. The crude product was purified by silica gel column chromatography to afford allyl 5-((1S)-fluoro((((S)-1-oxo-1-propoxypropan-2-yl)amino)(2,2,2-trifluoroethoxy)phosphoryl)methyl)benzo[b]thiophene-2-carboxylate (2, 860 mg, 1.64 mmol, 41.6% yield) as a colorless oil. LCMS (ESI): m/z=526 [M+H]⁺.

### Step B: 5-((1S)-fluoro((((S)-1-oxo-1-propoxypropan-2-yl)amino)(2,2,2-trifluoroethoxy)phosphoryl)methyl)benzo[b]thiophene-2-carboxylic acid

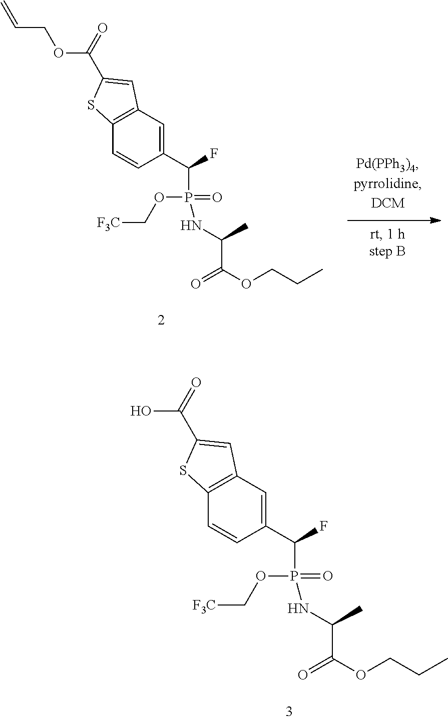 [SMILES: C=CCOC(=O)c1cc2cc([C@@H](F)P(=O)(N[C@@H](C)C(=O)OCCC)OCC(F)(F)F)ccc2s1 ; CCCOC(=O)[C@H](C)NP(=O)(OCC(F)(F)F)[C@H](F)c1ccc2sc(C(=O)O)cc2c1]

To a solution of allyl 5-((1S)-fluoro((((S)-1-oxo-1-propoxypropan-2-yl)amino)(2,2,2-trifluoroethoxy)phosphoryl)methyl)benzo[b]thiophene-2-carboxylate (2, 860 mg, 1.64 mmol, 1 eq.) in DCM (10 mL) was added Pd(PPh₃)₄ (184 mg, 0.16 mmol, 0.1 eq.), and pyrrolidine (116 mg, 1.64 mmol, 1 eq.). The resulting mixture was stirred for 1 h at room temperature under N₂ atmosphere. The mixture was adjusted to pH=3-4 with 1 M HCl (aq.), then extracted with DCM (20 mL×3), and the organic layers were combined and washed with brine (20 mL×2) dried over Na₂SO₄, filtered, and concentrated to dryness under reduced pressure to afford crude product 5-((1S)-fluoro((((S)-1-oxo-1-propoxypropan-2-yl)amino)(2,2,2-trifluoroethoxy)phosphoryl)methyl)benzo[b]thiophene-2-carboxylic acid (3, 650 mg, 1.34 mmol, 81.7% yield) as a yellow solid. LCMS (ESI): m/z=486 [M+H]⁺.

### Step C: perfluorophenyl 5-((1S)-fluoro((((S)-1-oxo-1-propoxypropan-2-yl)amino)(2,2,2-trifluoroethoxy)phosphoryl)methyl)benzo[b]thiophene-2-carboxylate

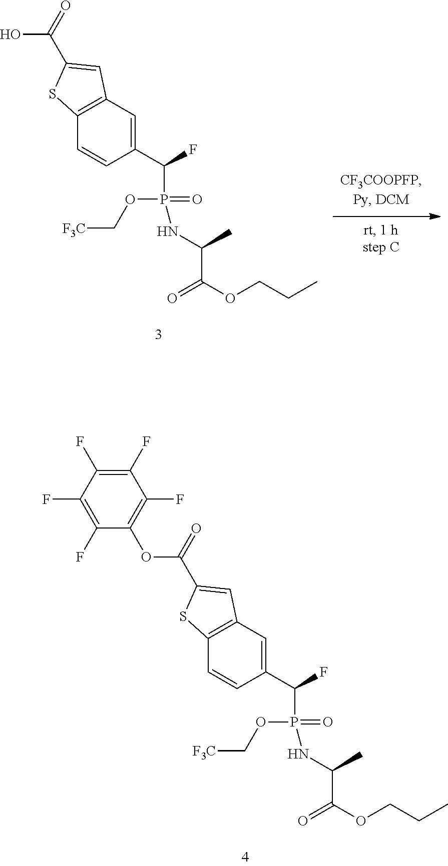 [SMILES: CCCOC(=O)[C@H](C)NP(=O)(OCC(F)(F)F)[C@H](F)c1ccc2sc(C(=O)O)cc2c1 ; CCCOC(=O)[C@H](C)NP(=O)(OCC(F)(F)F)[C@H](F)c1ccc2sc(C(=O)Oc3c(F)c(F)c(F)c(F)c3F)cc2c1]

To a solution of 5-((1S)-fluoro((((S)-1-oxo-1-propoxypropan-2-yl)amino)(2,2,2-trifluoroethoxy)phosphoryl)methyl)benzo[b]thiophene-2-carboxylic acid (3, 650 mg, 1.34 mmol, 1 eq.) in DCM (10 mL) was added pyridine (317 mg, 4.02 mmol, 3 eq.), perfluorophenyl 2,2,2-trifluoroacetate (750 mg, 2.68 mmol, 2 eq.). The resulting mixture was stirred for 1 h at room temperature and H₂O (20 mL) was added to quench the reaction, which was then extracted with DCM (30 mL×2). The organic layers were combined and washed with brine (20 mL×2), dried over Na₂SO₄, and concentrated to dryness under reduced pressure. The crude product was purified by silica gel column chromatography to afford perfluorophenyl 5-((1S)-fluoro((((S)-1-oxo-1-propoxypropan-2-yl)amino)(2,2,2-trifluoroethoxy)phosphoryl)methyl)benzo[b]thiophene-2-carboxylate (4, 700 mg, 1.08 mmol, 80% yield) as a white solid. LCMS (ESI): m/z=652 [M+H]⁺.

### Step D: propyl (((S)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(2,2,2-trifluoroethoxy)phosphoryl)-L-alaninate

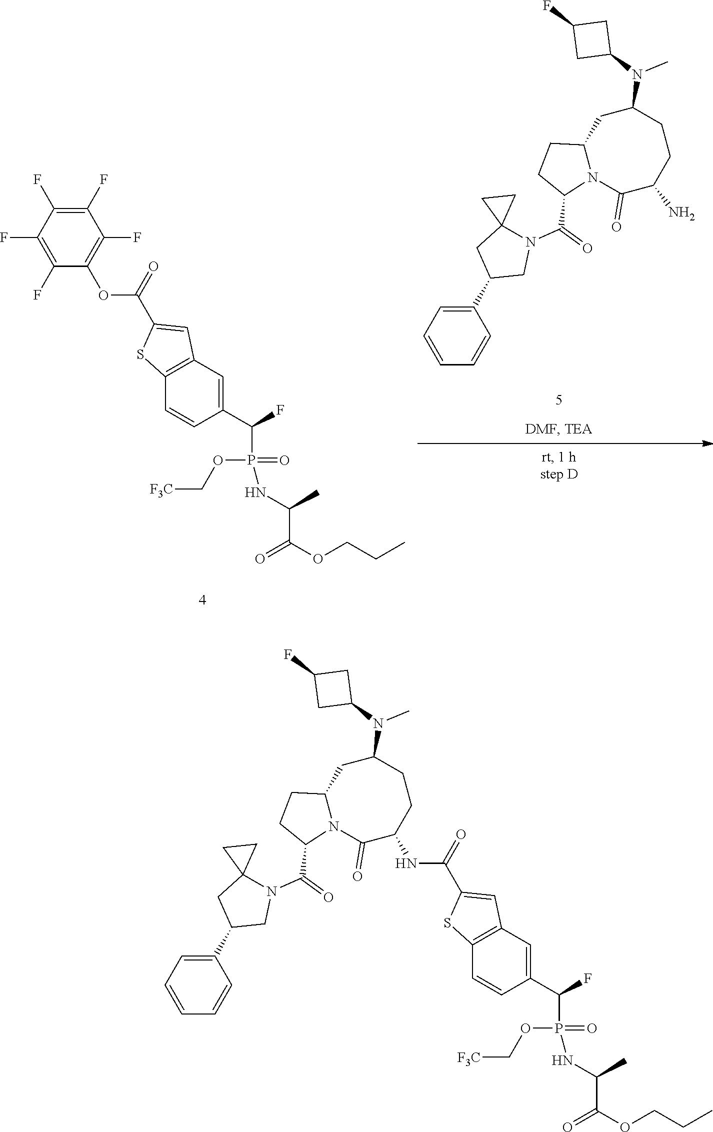 [SMILES: CCCOC(=O)[C@H](C)NP(=O)(OCC(F)(F)F)[C@H](F)c1ccc2sc(C(=O)Oc3c(F)c(F)c(F)c(F)c3F)cc2c1 ; CN([C@H]1CC[C@H](N)C(=O)N2[C@H](CC[C@H]2C(=O)N2C[C@H](c3ccccc3)CC23CC3)C1)[C@H]1C[C@@H](F)C1 ; CCCOC(=O)[C@H](C)NP(=O)(OCC(F)(F)F)[C@H](F)c1ccc2sc(C(=O)N[C@H]3CC[C@H](N(C)[C@H]4C[C@@H](F)C4)C[C@H]4CC[C@@H](C(=O)N5C[C@H](c6ccccc6)CC56CC6)N4C3=O)cc2c1]

To a solution of perfluorophenyl 5-((1S)-fluoro((((S)-1-oxo-1-propoxypropan-2-yl)amino)(2,2,2-trifluoroethoxy)phosphoryl)methyl)benzo[b]thiophene-2-carboxylate (4, 30 mg, 0.046 mml, 1 eq.) in DMF (3 mL) was added (3S,6S,9S,10aR)-6-amino-9-(((1s,3R)-3-fluorocyclobutyl)(methyl)amino)-3-((S)-6-phenyl-4-azaspiro[2.4]heptane-4-carbonyl)octahydropyrrolo[1,2-a]azocin-5(1H)-one (5, 22 mg, 0.046 mmol, 1 eq.), TEA (14 mg, 0.138 mmol, 3 eq.). The resulting mixture was stirred for 1 h at room temperature. The crude product was purified by Prep-HPLC to afford propyl (((S)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(2,2,2-trifluoroethoxy)phosphoryl)-L-alaninate (Compound C64), 19 mg, 0.020 mmol, 45.6% yield) as a white solid. LCMS (ESI): m/z=950 [M+H]⁺. ¹H NMR (400 MHz, DMSO-d₆) δ (ppm) 8.86-8.75 (m, 1H), 8.30-8.22 (m, 1H), 8.10-8.02 (m, 1H), 8.01-7.95 (m, 1H), 7.56-7.47 (m, 1H), 7.37-7.27 (m, 4H), 7.24-7.18 (m, 1H), 6.27-5.91 (m, 2H), 4.92-4.68 (m, 2H), 4.66-4.42 (m, 3H), 4.36-4.24 (m, 1H), 4.16-4.08 (m, 1H), 4.05-3.93 (m, 2H), 3.84-3.50 (m, 3H), 3.08-2.93 (m, 1H), 2.64-2.56 (m, 1H), 2.44-2.31 (m, 2H), 2.30-1.45 (m, 21H), 1.31-1.01 (m, 3H), 0.89-0.82 (m, 3H), 0.58-0.39 (m, 2H). ¹⁹F NMR (376 MHz, DMSO-d₆) δ (ppm) −73.67 (d, J=22.8 Hz), −165.29 (s), −195.24 (d, J=83.7 Hz), −197.01 (d, J=86.2 Hz). ³¹P NMR (162 MHz, DMSO-d6) δ (ppm) 21.32 (d, J=84.0 Hz), 20.98 (d, J=86.2 Hz).

### Synthesis of ((R)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A4) and propyl ((R)-((1R)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)-2,3-dihydrobenzo[b]thiophen-5-yl)methyl)(phenoxy)phosphoryl)-L-alaninate (Compound C5)

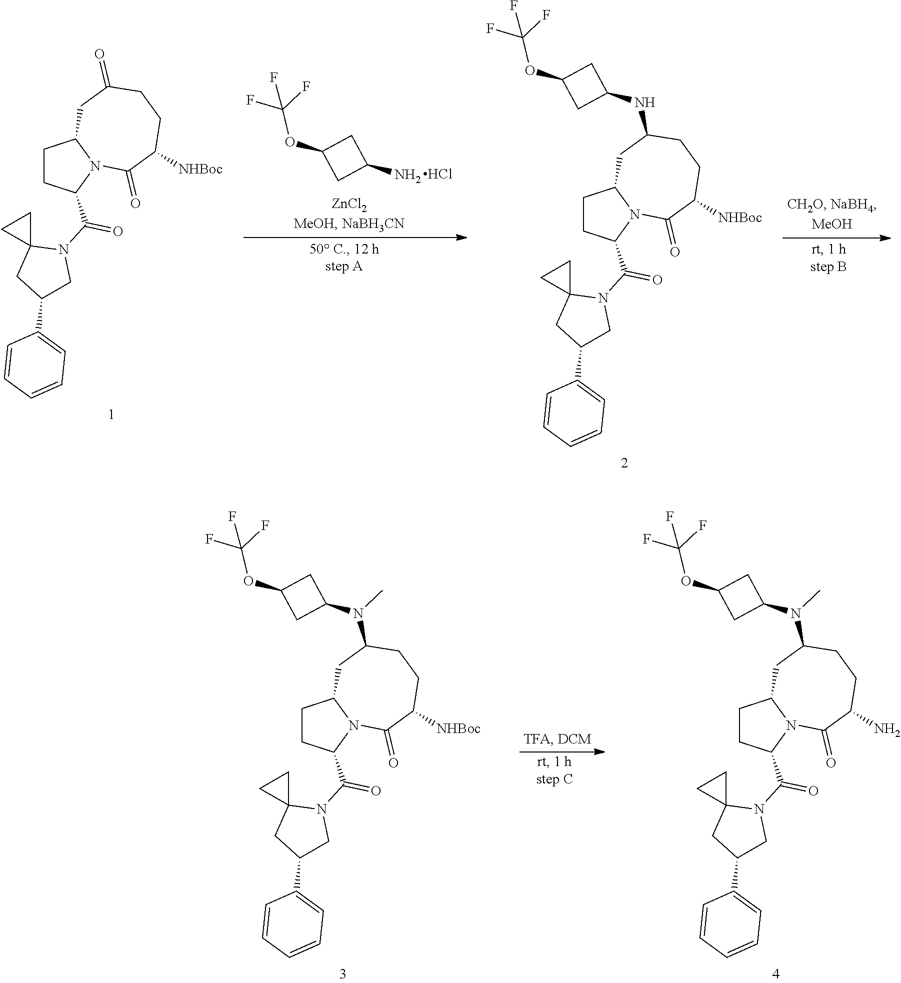 [SMILES: CC(C)(C)OC(=O)N[C@H]1CCC(=O)C[C@H]2CC[C@@H](C(=O)N3C[C@H](c4ccccc4)CC34CC4)N2C1=O ; N[C@H]1C[C@@H](OC(F)(F)F)C1 ; Cl ; CC(C)(C)OC(=O)N[C@H]1CC[C@H](N[C@H]2C[C@@H](OC(F)(F)F)C2)C[C@H]2CC[C@@H](C(=O)N3C[C@H](c4ccccc4)CC34CC4)N2C1=O ; CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](OC(F)(F)F)C1 ; CN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1]

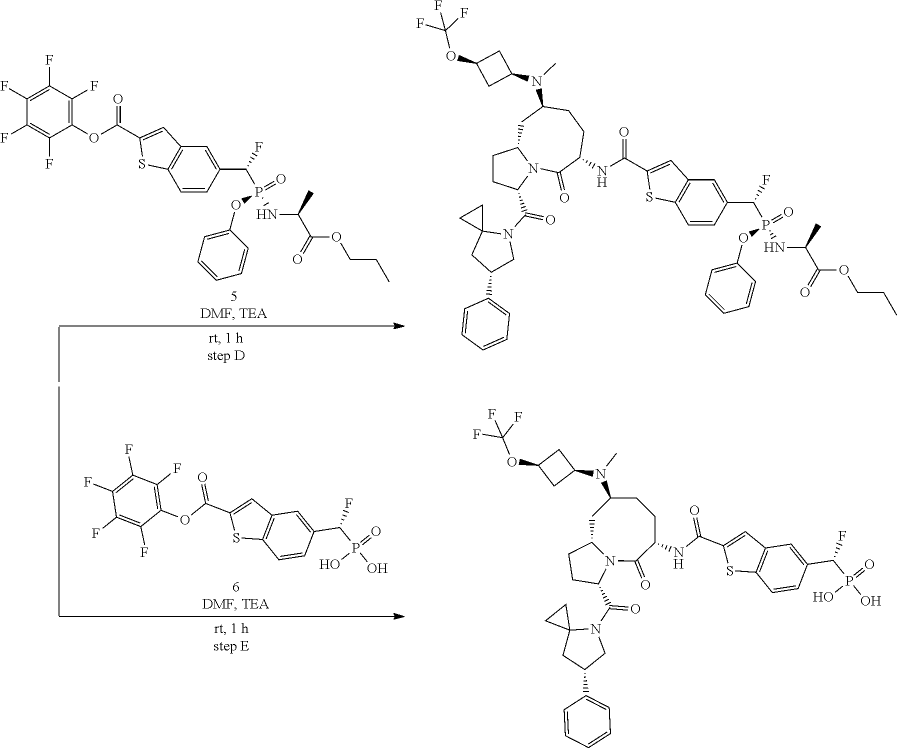 [SMILES: CCCOC(=O)[C@H](C)N[P@](=O)(Oc1ccccc1)[C@@H](F)c1ccc2sc(C(=O)Oc3c(F)c(F)c(F)c(F)c3F)cc2c1 ; CCCOC(=O)[C@H](C)N[P@](=O)(Oc1ccccc1)[C@@H](F)c1ccc2sc(C(=O)N[C@H]3CC[C@H](N(C)[C@H]4C[C@@H](OC(F)(F)F)C4)C[C@H]4CC[C@@H](C(=O)N5C[C@H](c6ccccc6)CC56CC6)N4C3=O)cc2c1 ; O=C(Oc1c(F)c(F)c(F)c(F)c1F)c1cc2cc([C@H](F)P(=O)(O)O)ccc2s1 ; CN([C@H]1CC[C@H](NC(=O)c2cc3cc([C@H](F)P(=O)(O)O)ccc3s2)C(=O)N2[C@H](CC[C@H]2C(=O)N2C[C@H](c3ccccc3)CC23CC3)C1)[C@H]1C[C@@H](OC(F)(F)F)C1]

### Step A: tert-butyl ((3S,6S,9S,10aR)-5-oxo-3-((S)-6-phenyl-4-azaspiro[2.4]heptane-4-carbonyl)-9-(((1 s, 3R)-3-(trifluoromethoxy)cyclobutyl)amino)decahydropyrrolo[1,2-a]azocin-6-yl)carbamate

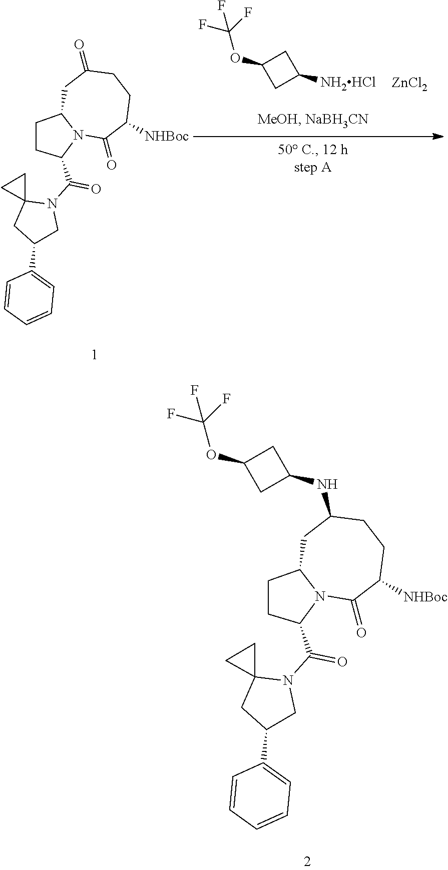

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (1, 1.5 g, 3.03 mmol, 1 eq.) in MeOH (20 mL) was added (1s,3s)-3-(trifluoromethoxy)cyclobutan-1-amine hydrochloride (868 mg, 4.55 mml, 1.5 eq.), ZnCl₂ (4.13 g, 30.3 mmol, 10 eq.). The mixture was stirred for 1 h at 50° C., then NaBH₃CN (1.91 g, 30.3 mmol, 10 eq.) added portion wise every 30 mins (2 eq. (382 mg each time) added 5 times). The mixture was stirred for 12 h at 50° C., then cooled to room temperature. The resulting mixture was used in the next step directly without further workup. LCMS (ESI): m/z=635 [M+H]⁺.

### Step B: tert-butyl ((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamate

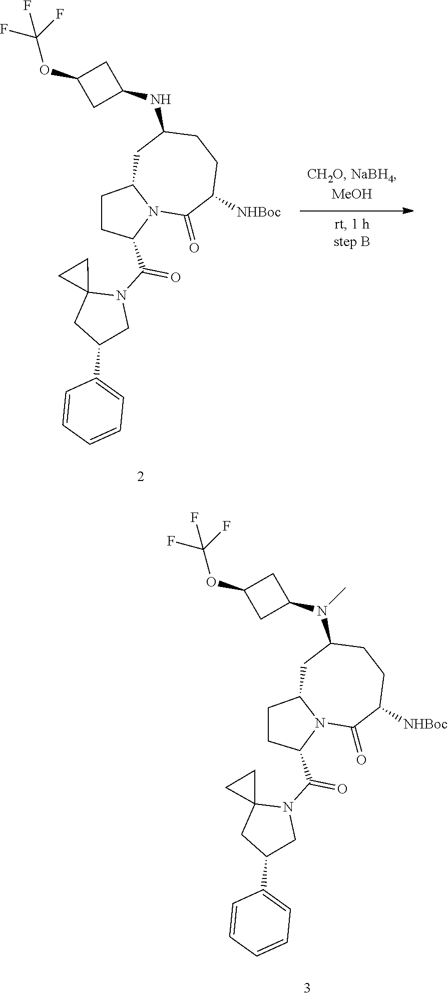 [SMILES: CC(C)(C)OC(=O)N[C@H]1CC[C@H](N[C@H]2C[C@@H](OC(F)(F)F)C2)C[C@H]2CC[C@@H](C(=O)N3C[C@H](c4ccccc4)CC34CC4)N2C1=O ; CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](OC(F)(F)F)C1]

To a above mixture (step A) was added 40% formaldehyde (1 mL) at room temperature, the mixture was stirred for 10 min, then NaBH₄ (1.15 g, 30.3 mmol, 10 eq.) was added to the mixture in several portions during 20 mins, stirred for additional 30 mins, LCMS show that the reaction was completed. The reaction was quenched by NH₄Cl (sat. aq.), diluted with H₂O (200 mL) and extracted with EtOAc (100 mL×3). The organic layers were dried over Na₂SO₄, concentrated to dryness under reduced pressure, and the crude product was purified by C18 column (MeCN:H₂O (0.1% ammonium hydroxide)=5%-70%) to afford tert-butyl ((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamate (3, 1 g, 1.54 mmol, 76.9% yield, two steps) as a white solid. LCMS (ESI): m/z=649 [M+H]⁺.

### Step C: (3S,6S,9S,10aR)-6-amino-9-(methyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one

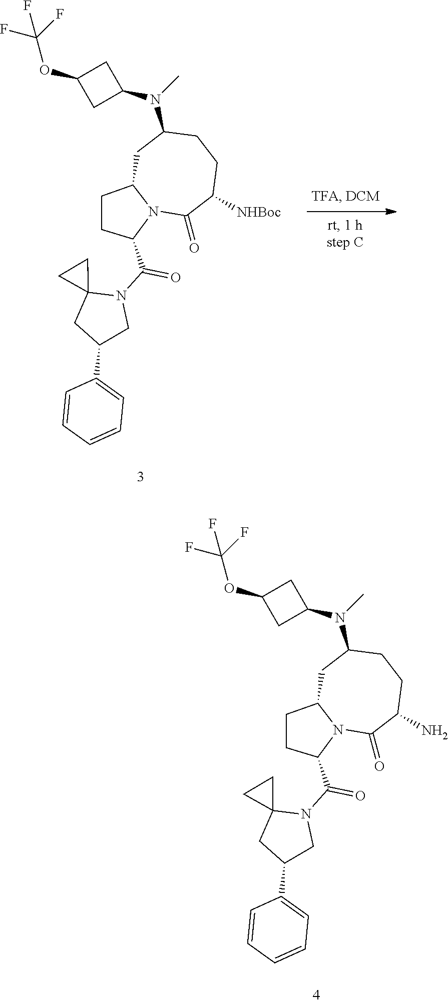 [SMILES: CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](OC(F)(F)F)C1 ; CN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1]

To a solution of tert-butyl ((3S,6S,9S,10aR)-9-methyl(1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamate (3, 30 mg, 0.046 mmol) in DCM (2 mL) was added TFA (2 mL), The resulting mixture was stirred for 1 h at room temperature and was concentrated under reduced pressure to afford crude product (3S,6S,9S,10aR)-6-amino-9-(methyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one (4, 30 mg, TFA salt) as a yellow oil which used in the next step without further purification. LCMS (ESI): m/z=549 [M+H]⁺.

### Step D: propyl ((R)-((R)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

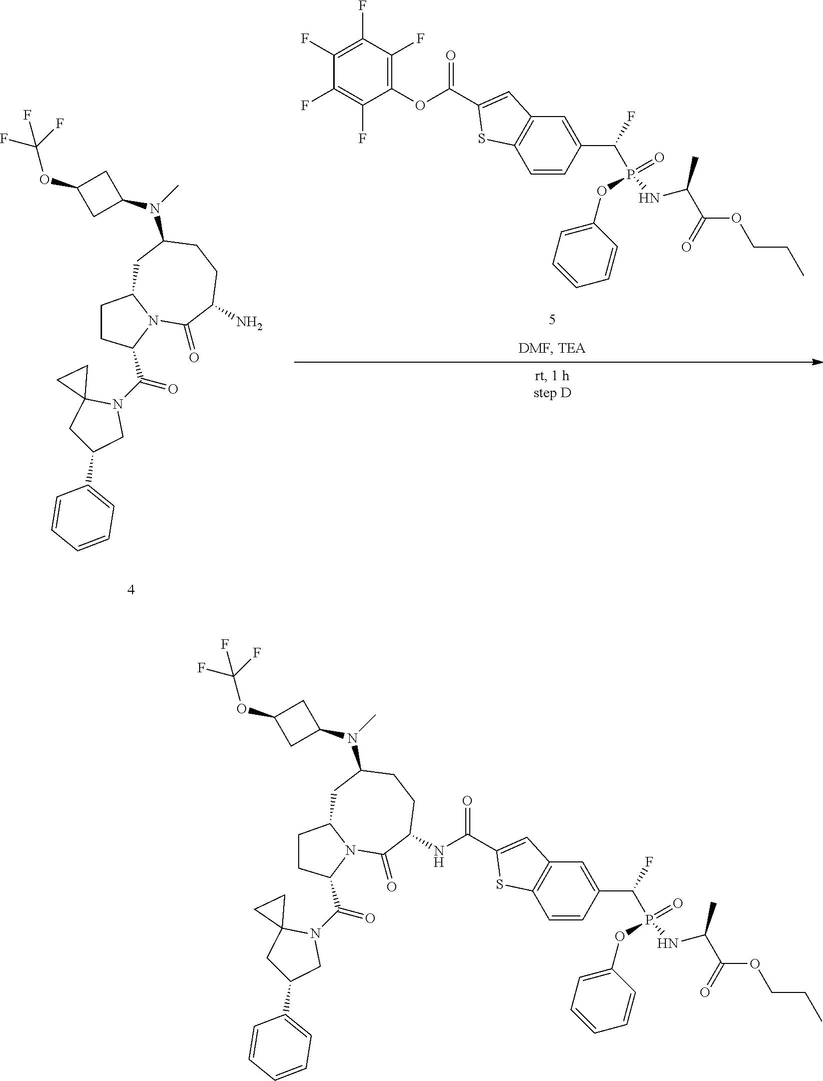 [SMILES: CN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1 ; CCCOC(=O)[C@H](C)N[P@](=O)(Oc1ccccc1)[C@@H](F)c1ccc2sc(C(=O)Oc3c(F)c(F)c(F)c(F)c3F)cc2c1 ; CCCOC(=O)[C@H](C)N[P@](=O)(Oc1ccccc1)[C@@H](F)c1ccc2sc(C(=O)N[C@H]3CC[C@H](N(C)[C@H]4C[C@@H](OC(F)(F)F)C4)C[C@H]4CC[C@@H](C(=O)N5C[C@H](c6ccccc6)CC56CC6)N4C3=O)cc2c1]

To a solution of (3S,6S,9S,10aR)-6-amino-9-(methyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one (4, 30 mg, TFA salt) in DMF (2 mL) was added TEA (9.4 mg, 0.093 mmol, 2 eq.), perfluorophenyl 5-((R)-fluoro((R)-(((S)-1-oxo-1-propoxypropan-2-yl)amino)(phenoxy)phosphoryl)methyl)benzo[b]thiophene-2-carboxylate (5, 30 mg, 0.046 mmol, 1 eq). The resulting mixture was stirred for 1 h at room temperature and the crude product was purified by Prep-HPLC to afford propyl ((R)-((R)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C5, 24 mg, 0.024 mmol) as a white solid. LCMS (ESI): m/z=1010.7 [M+H]⁺. ¹H NMR (400 MHz, DMSO-d₆) δ (ppm) 8.79 (d, J=7.6 Hz, 1H), 8.27 (s, 1H), 8.13-7.98 (m, 2H), 7.60 (d, J=8.6 Hz, 1H), 7.40-7.26 (m, 6H), 7.23-7.12 (m, 4H), 6.29-6.03 (m, 2H), 4.71 (t, J=8.5 Hz, 1H), 4.57-4.43 (m, 2H), 4.34-4.24 (m, 1H), 4.12 (t, J=8.7 Hz, 1H), 3.87-3.68 (m, 4H), 3.60-3.47 (m, 1H), 3.08-2.92 (m, 1H), 2.72-2.58 (m, 2H), 2.46-2.09 (m, 5H), 2.03-1.92 (m, 6H), 1.90-1.47 (m, 9H), 1.46-1.35 (m, 2H), 1.13 (d, J=7.1 Hz, 3H), 0.77 (t, J=7.4 Hz, 3H), 0.56-0.37 (m, 2H). ¹⁹F NMR (376 MHz, DMSO-d₆) δ (ppm) −57.57 (s), −194.28 (d, J=83.0 Hz). ³¹P NMR (162 MHz, DMSO-d₆) δ (ppm) 17.32 (d, J=83.5 Hz).

### Step E: ((R)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

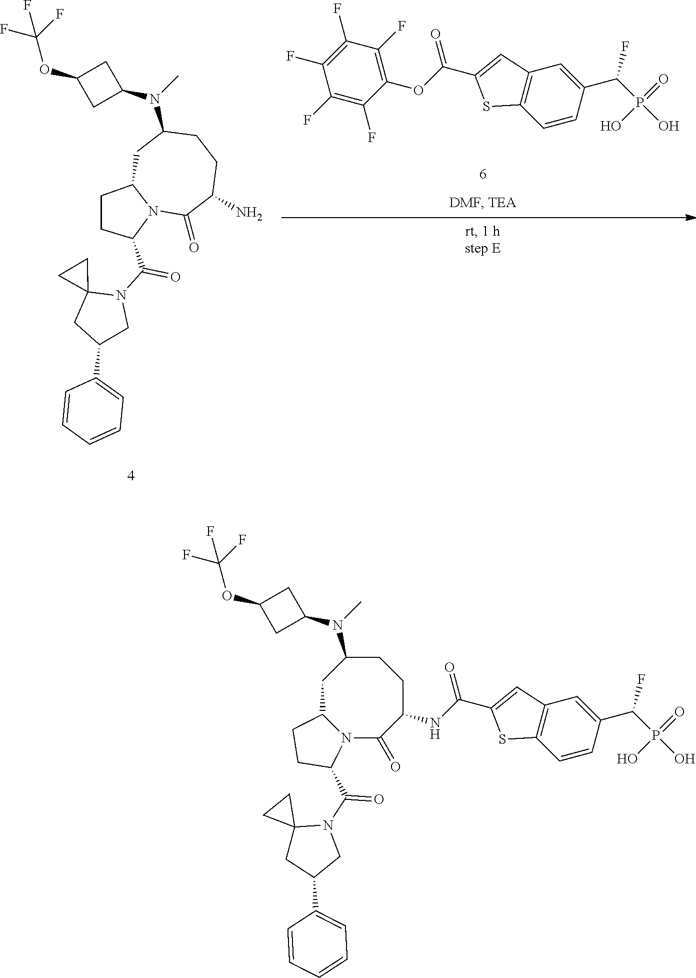

To a solution of (3S,6S,9S,10aR)-6-amino-9-(methyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one (4, 30 mg, TFA salt) in DMF (2 mL) was added TEA (9.4 mg, 0.093 mmol, 2 eq.), (R)-(fluoro(2-((perfluorophenoxy)carbonyl)benzo[b]thiophen-5-yl)methyl)phosphonic acid (6, 21.1 mg, 0.046 mmol, 1 eq.). The resulting mixture was stirred for 1 h at room temperature, and the crude product was purified by Prep-HPLC to afford ((R)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A4, 12 mg, 0.015 mmol) as a white solid. LCMS (ESI): m/z=821.2 [M+H]⁺. 1H NMR (400 MHz, CD₃OD-d₄) δ (ppm) 8.13-8.00 (m, 2H), 7.92 (d, J=8.5 Hz, 1H), 7.70 (d, J=8.5 Hz, 1H), 7.34-7.15 (m, 5H), 5.79-5.60 (m, 1H), 4.67-4.46 (m, 3H), 4.40-4.28 (m, 1H), 3.99-3.75 (m, 2H), 3.75-3.64 (m, 1H), 3.57-3.42 (m, 1H), 3.05-2.91 (m, 1H), 2.87-1.19 (m, 21H), 0.56-0.33 (m, 2H). ¹⁹F NMR (376 MHz, CD₃OD-d₄) δ (ppm) −61.08 (s), −195.27 (d, J=77.8 Hz). ³¹P NMR (162 MHz, CD₃OD-d₄) δ (ppm) 9.67 (d, J=76.4 Hz).

### Synthesis of ((S)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A5)

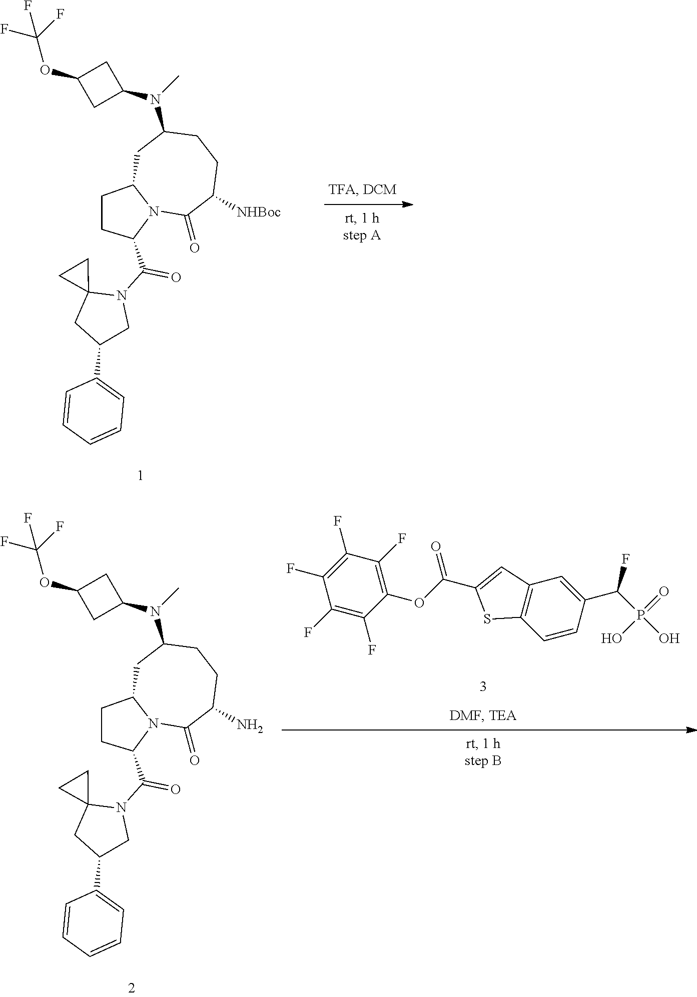 [SMILES: CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](OC(F)(F)F)C1 ; CN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1 ; O=C(Oc1c(F)c(F)c(F)c(F)c1F)c1cc2cc([C@@H](F)P(=O)(O)O)ccc2s1]

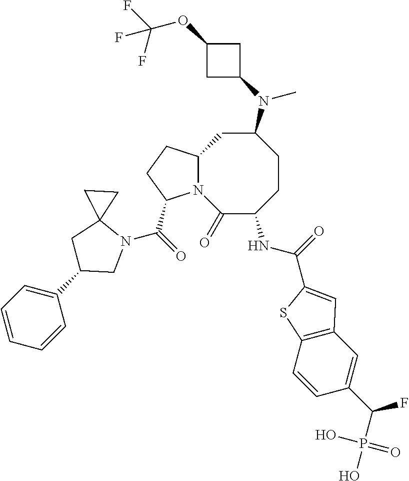 [SMILES: CN([C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1)[C@H]1C[C@@H](OC(F)(F)F)C1]

### Step A: (3S,6S,9S,10aR)-6-amino-9-(methyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one

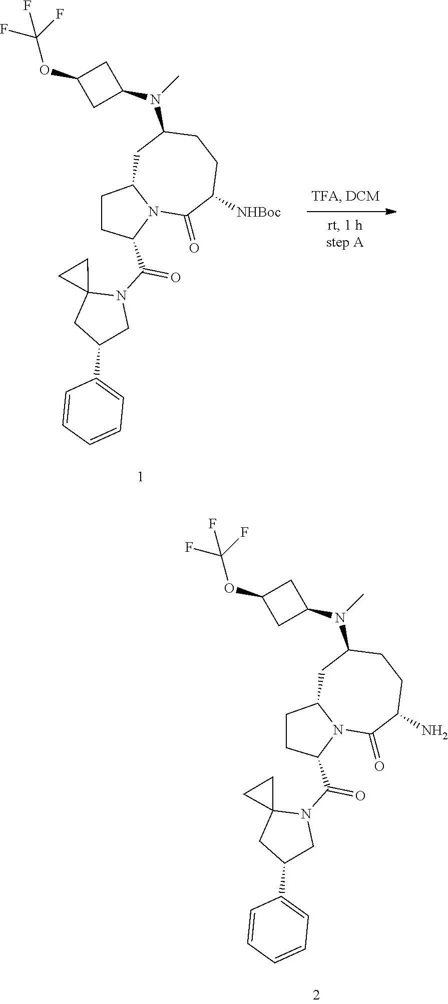 [SMILES: CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](OC(F)(F)F)C1 ; CN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1]

To a solution of tert-butyl ((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamate (1, 30 mg, 0.046 mmol) in DCM (2 mL) was added TFA (2 mL), The resulting mixture was stirred for 1 h at room temperature. The resulting solution was concentrated under reduced pressure to afford crude product (3S,6S,9S,10aR)-6-amino-9-(methyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one (2, 30 mg, TFA salt) as a yellow oil which used in the next step without further purification. LCMS (ESI): m/z=549 [M+H]⁺.

### Step B: ((S)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

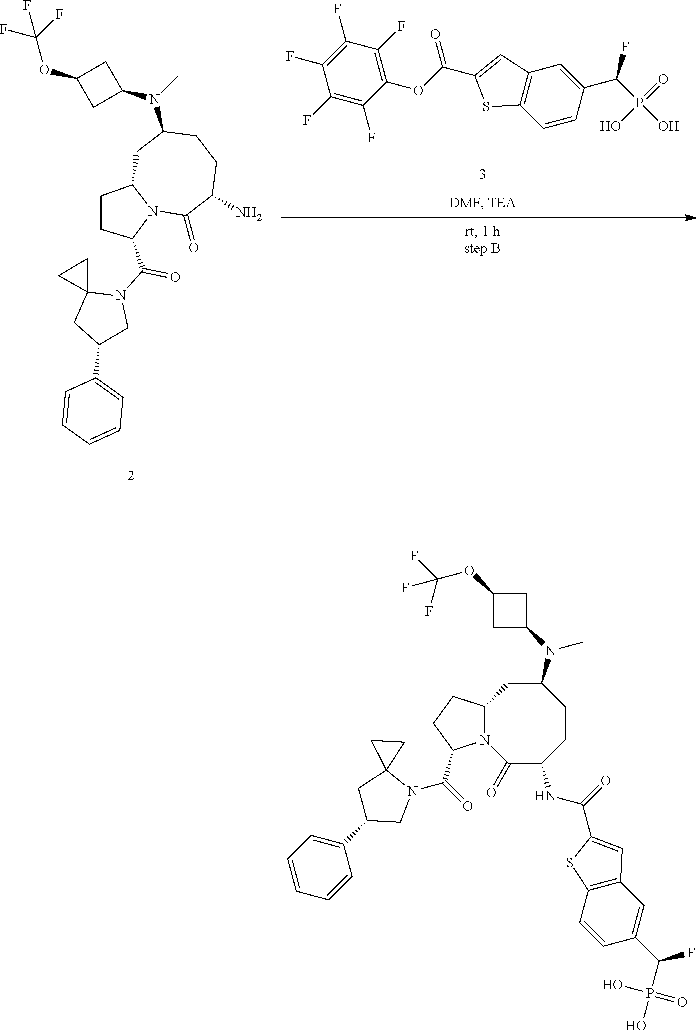 [SMILES: CN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1 ; O=C(Oc1c(F)c(F)c(F)c(F)c1F)c1cc2cc([C@@H](F)P(=O)(O)O)ccc2s1 ; CN([C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1)[C@H]1C[C@@H](OC(F)(F)F)C1]

To a solution of (3S,6S,9S,10aR)-6-amino-9-(methyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one (2, 30 mg, TFA salt) in DMF (2 mL) was added TEA (9.4 mg, 0.093 mmol, 2 eq.), (S)-(fluoro(2-((perfluorophenoxy)carbonyl)benzo[b]thiophen-5-yl)methyl)phosphonic acid (3, 21.1 mg, 0.046 mmol, 1 eq.). The resulting mixture was stirred for 1 h at room temperature, and the crude product was purified by Prep-HPLC to afford ((S)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A5, 18 mg, 0.022 mmol) as a white solid. LCMS (ESI): m/z=821.3 [M+H]⁺. ¹H NMR (400 MHz, CD₃OD-d₄) δ (ppm) 8.12-8.01 (m, 2H), 7.87 (d, J=8.4 Hz, 1H), 7.62 (d, J=8.5 Hz, 1H), 7.35-7.26 (m, 4H), 7.20 (t, J=7.0 Hz, 1H), 5.78-5.54 (m, 1H), 4.67 (d, J=10.1 Hz, 1H), 4.59-4.48 (m, 2H), 4.42-4.35 (m, 1H), 3.99 (t, J=8.6 Hz, 1H), 3.88 (t, J=9.7 Hz, 1H), 3.63-3.46 (m, 2H), 3.23-3.17 (m, 1H), 2.91-2.81 (m, 1H), 2.71-2.61 (m, 1H), 2.60-2.50 (m, 1H), 2.40-1.57 (m, 21H), 0.54-0.41 (m, 2H). ¹⁹F NMR (376 MHz, CD₃OD-d₄) δ (ppm) −60.66-−61.44 (m), −196.29 (d, J=77.8 Hz). ³¹P NMR (162 MHz, CD₃OD-d₄) δ (ppm) 9.73 (d, J=75.8 Hz).

### Synthesis of isobutyl (cyclopropoxy((S)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphoryl)-L-alaninate (Compound C156)

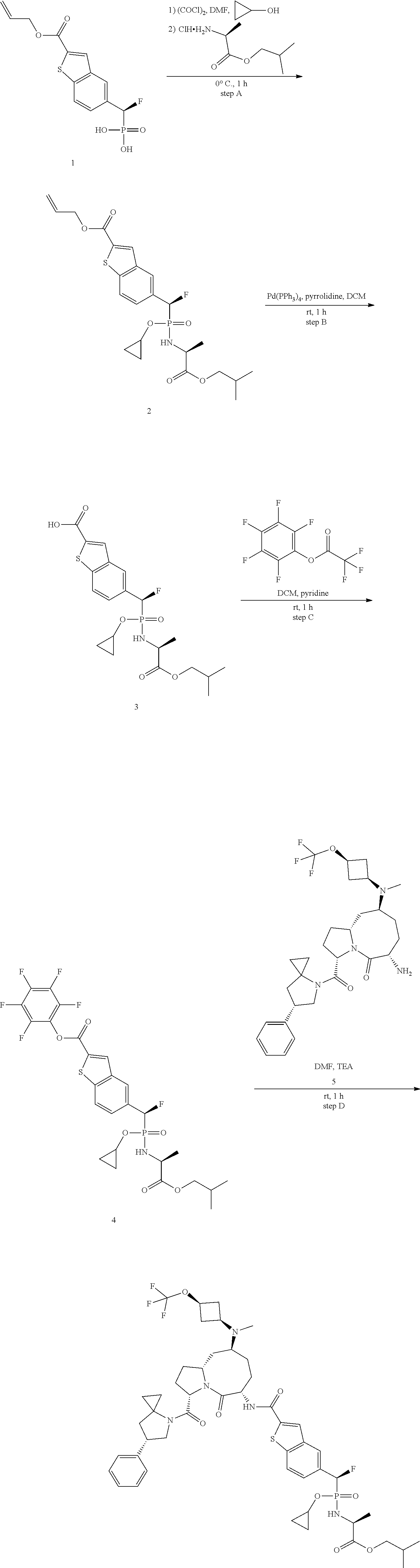

### Step A: allyl 5-((1S)-(cyclopropoxy(((S)-1-isobutoxy-1-oxopropan-2-yl)amino)phosphoryl)fluoromethyl)benzo[b]thiophene-2-carboxylate

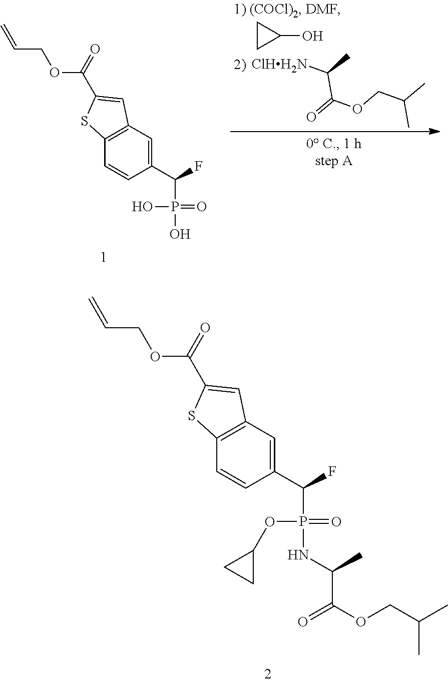

To a solution of (S)-((2-((allyloxy)carbonyl)benzo[b]thiophen-5-yl)fluoromethyl)phosphonic acid (1, 1 g, 3.03 mmol, 1 eq.) in DCM (20 mL) was added DMF (1 drop), and oxalyl chloride (1.92 g, 15.15 mmol, 5 eq.). The resulting mixture was stirred for 1 h at room temperature, then DCM was evaporated to dryness under reduced pressure to remove the excess oxalyl chloride, then redissolved in dry DCM (20 mL), the solution was cooled down to 0° C., cyclopropanol (176 mg, 3.03 mmol, 1 eq.) was added, followed by TEA (1.53 g, 15.15 mmol, 5 eq.) dropwise. The resulting mixture was stirred for 1 h at 0° C., then isobutyl L-alaninate hydrochloride (1.1 g, 6.06 mmol, 2 eq.) was added to the solution, then stirred for 10 min at 0° C. The reaction was quenched by adding H₂O (20 mL), then extracted with DCM (20 mL×2), the organic layers were combined and washed with brine (20 mL), then dried over Na₂SO₄, then concentrated under reduced pressure, the crude product was purified by silica gel column to afford allyl 5-((1S)-(cyclopropoxy(((S)-1-isobutoxy-1-oxopropan-2-yl)amino)phosphoryl)fluoromethyl)benzo[b]thiophene-2-carboxylate (2, 500 mg, 1.01 mmol, 33% yield) as a white solid. LCMS (ESI): m/z=498 [M+H]⁺.

### Step B: 5-((1S)-(cyclopropoxy(((S)-1-isobutoxy-1-oxopropan-2-yl)amino)phosphoryl)fluoromethyl)benzo[b]thiophene-2-carboxylic acid

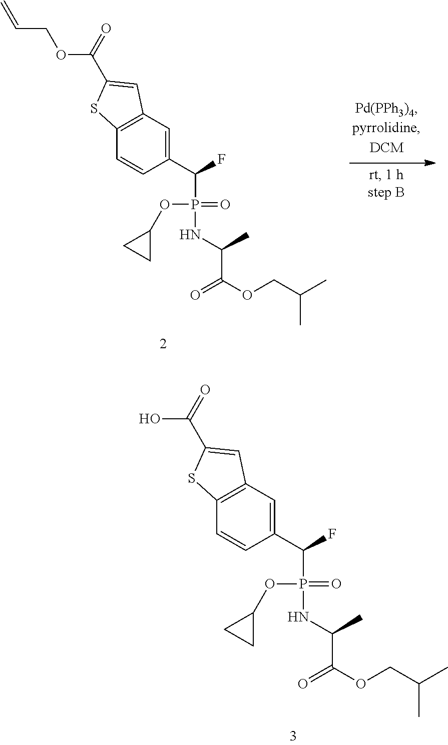 [SMILES: C=CCOC(=O)c1cc2cc([C@@H](F)P(=O)(N[C@@H](C)C(=O)OCC(C)C)OC3CC3)ccc2s1 ; CC(C)COC(=O)[C@H](C)NP(=O)(OC1CC1)[C@H](F)c1ccc2sc(C(=O)O)cc2c1]

To a solution of allyl 5-((1S)-(cyclopropoxy(((S)-1-isobutoxy-1-oxopropan-2-yl)amino)phosphoryl)fluoromethyl)benzo[b]thiophene-2-carboxylate (2, 500 mg, 1.01 mmol, 1 eq.) in DCM (10 mL) was added Pd(PPh₃)₄ (115 mg, 0.1 mmol, 0.1 eq.), pyrrolidine (71 mg, 1.01 mmol, 1 eq.). The resulting mixture was stirred for 1 h at room temperature under the atmosphere of N₂. The reaction was quenched by adding H₂O (10 mL), and adjusted pH=3 with 1 M HCl (aq.). Then extracted with DCM (10 mL×3), the organic layers were combined and washed with brine (20 mL), dried over Na₂SO₄, then concentrated to dryness under reduced pressure to afford crude product 5-((1S)-(cyclopropoxy(((S)-1-isobutoxy-1-oxopropan-2-yl)amino)phosphoryl)fluoromethyl)benzo[b]thiophene-2-carboxylic acid (3, 450 mg, 0.98 mmol, 97% yield) as a yellow solid, which was used in next step without further purification. LCMS (ESI): m/z=458 [M+H]⁺.

### Step C: perfluorophenyl 5-((1S)-(cyclopropoxy(((S)-1-isobutoxy-1-oxopropan-2-yl)amino)phosphoryl)fluoromethyl)benzo[b]thiophene-2-carboxylate

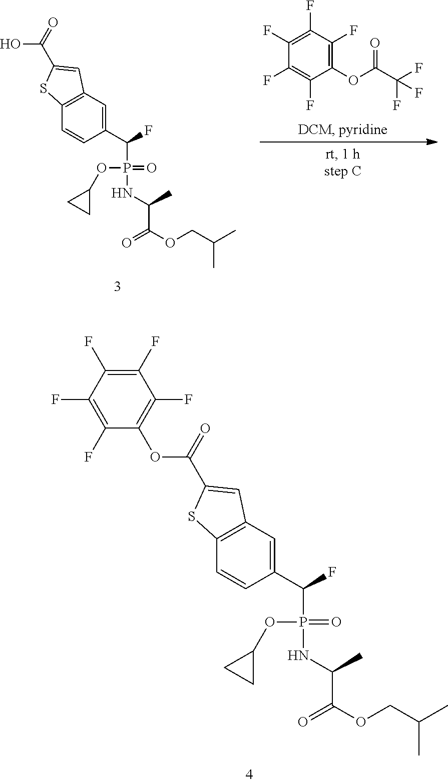 [SMILES: CC(C)COC(=O)[C@H](C)NP(=O)(OC1CC1)[C@H](F)c1ccc2sc(C(=O)O)cc2c1 ; O=C(Oc1c(F)c(F)c(F)c(F)c1F)C(F)(F)F ; CC(C)COC(=O)[C@H](C)NP(=O)(OC1CC1)[C@H](F)c1ccc2sc(C(=O)Oc3c(F)c(F)c(F)c(F)c3F)cc2c1]

To a solution of 5-((1S)-(cyclopropoxy(((S)-1-isobutoxy-1-oxopropan-2-yl)amino)phosphoryl)fluoromethyl)benzo[b]thiophene-2-carboxylic acid (3, 450 mg, 0.98 mmol, 1 eq.) in DCM (5 mL) was added pyridine (233 mg, 2.95 mmol, 3 eq.), perfluorophenyl 2,2,2-trifluoroacetate (276 mg, 1.97 mmol, 2 eq.). The resulting mixture was stirred for 1 h at room temperature, LCMS show that the reaction was completed. H₂O (10 mL) was added to the solution, which was adjusted to pH=3 with 1M HCl (aq.), then extracted with DCM (10 mL×3). The organic layers were combined and washed with brine, dried over Na₂SO₄, concentrated to dryness under reduced pressure the crude product was purified by C18 column to afford perfluorophenyl 5-((1S)-(cyclopropoxy(((S)-1-isobutoxy-1-oxopropan-2-yl)amino)phosphoryl)fluoromethyl)benzo[b]thiophene-2-carboxylate (4, 500 mg, 0.80 mmol, 81.6% yield) as a white solid. LCMS (ESI): m/z=624 [M+H]⁺.

### Step D: isobutyl (cyclopropoxy((S)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphoryl)-L-alaninate

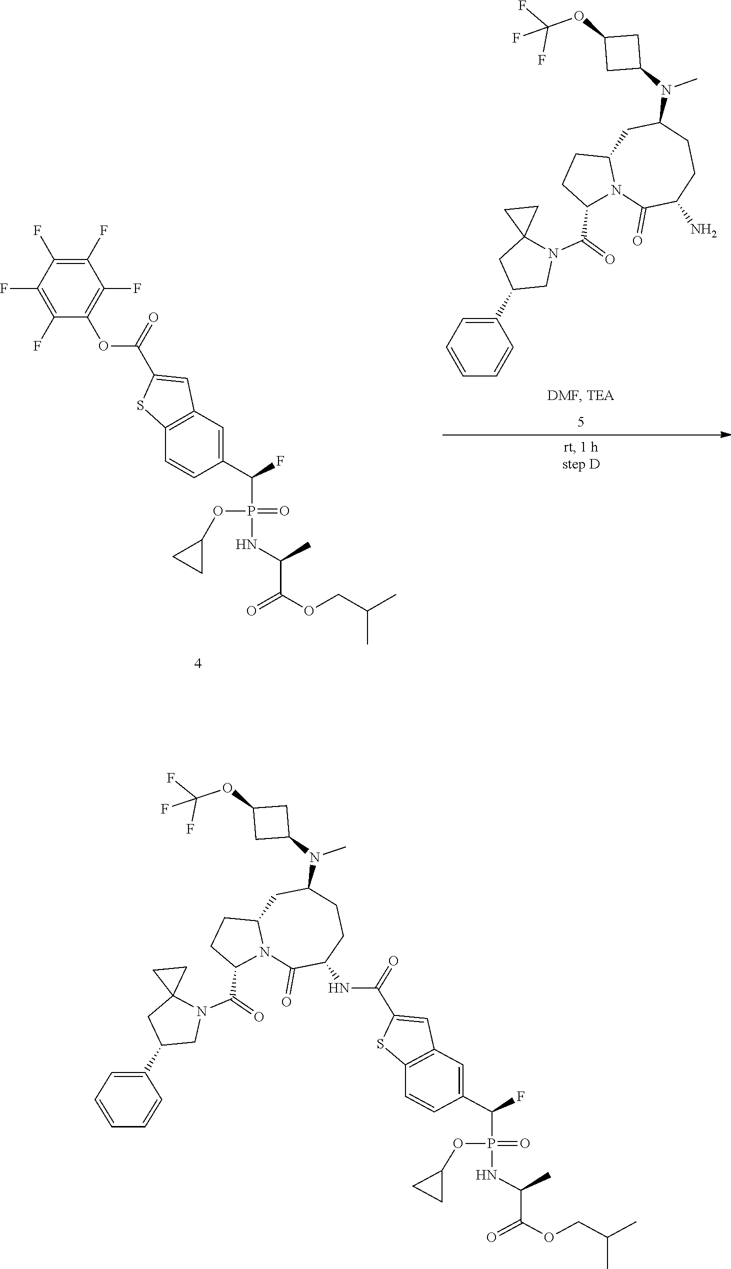 [SMILES: CC(C)COC(=O)[C@H](C)NP(=O)(OC1CC1)[C@H](F)c1ccc2sc(C(=O)Oc3c(F)c(F)c(F)c(F)c3F)cc2c1 ; CN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1 ; CC(C)COC(=O)[C@H](C)NP(=O)(OC1CC1)[C@H](F)c1ccc2sc(C(=O)N[C@H]3CC[C@H](N(C)[C@H]4C[C@@H](OC(F)(F)F)C4)C[C@H]4CC[C@@H](C(=O)N5C[C@H](c6ccccc6)CC56CC6)N4C3=O)cc2c1]

To a solution of perfluorophenyl 5-((1S)-(cyclopropoxy(((S)-1-isobutoxy-1-oxopropan-2-yl)amino)phosphoryl)fluoromethyl)benzo[b]thiophene-2-carboxylate (4, 30 mg, 0.048 mmol, 1 eq.) in DMF (2 mL) was added TEA (9.7 mg, 0.096 mmol, 2 eq.), (3S,6S,9S,10aR)-6-amino-9-(methyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one (5, 26 mg, 0.048 mmol, 1 eq.). The resulting mixture was stirred for 1 h at room temperature. The crude product was purified by Prep-HPLC to afford isobutyl (cyclopropoxy((S)-fluoro(2-(((3S,6S,9S,10aR)-9-(methyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphoryl)-L-alaninate (Compound C156, 26 mg, 0.026 mmol) as a white solid. LCMS (ESI): m/z=988 [M+H]⁺. ¹H NMR (400 MHz, DMSO-d₆) δ (ppm) 8.87-8.70 (m, 1H), 8.32-8.20 (m, 1H), 8.08-7.98 (m, 1H), 7.98-7.92 (m, 1H), 7.55-7.44 (m, 1H), 7.38-7.26 (m, 4H), 7.24-7.17 (m, 1H), 6.11-5.60 (m, 2H), 4.78-4.64 (m, 1H), 4.59-4.42 (m, 2H), 4.36-4.23 (m, 1H), 4.17-4.06 (m, 1H), 4.02-3.86 (m, 1H), 3.86-3.60 (m, 4H), 3.59-3.46 (m, 1H), 3.08-2.92 (m, 1H), 2.74-2.57 (m, 2H), 2.47-2.29 (m, 2H), 2.28-2.18 (m, 1H), 2.17-2.08 (m, 2H), 2.05-1.92 (m, 6H), 1.90-1.45 (m, 10H), 1.32-0.97 (m, 3H), 0.89-0.82 (m, 6H), 0.81-0.62 (m, 2H), 0.61-0.53 (m, 2H), 0.54-0.35 (m, 2H). ¹⁹F NMR (376 MHz, DMSO-d₆) δ (ppm) −57.57 (s), −193.97 (d, J=81.1 Hz), −196.49 (d, J=82.0 Hz). ³¹P NMR (162 MHz, DMSO-d₆) δ (ppm) 19.89 (d, J=82.2 Hz), 19.58 (d, J=80.7 Hz).

### Synthesis of ((R)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A2)

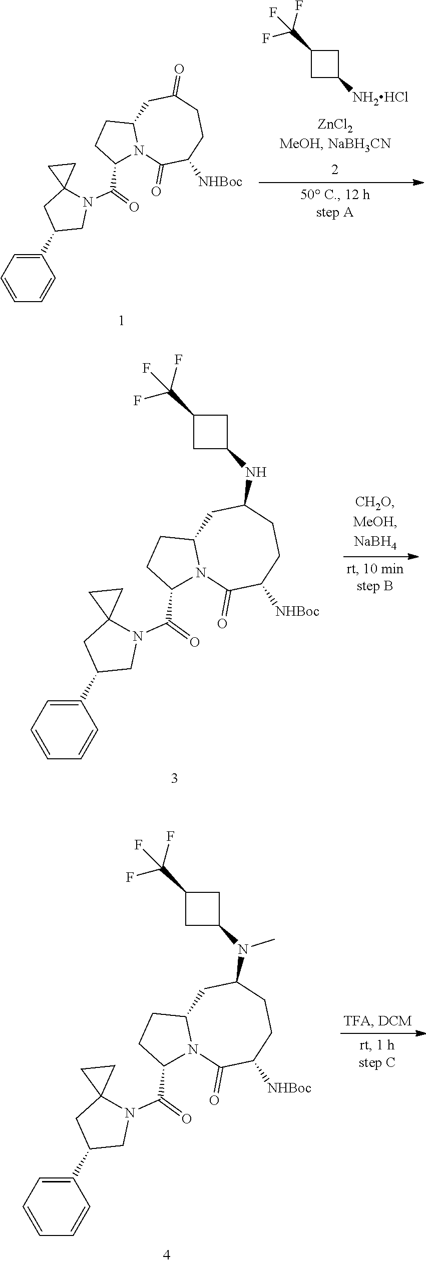

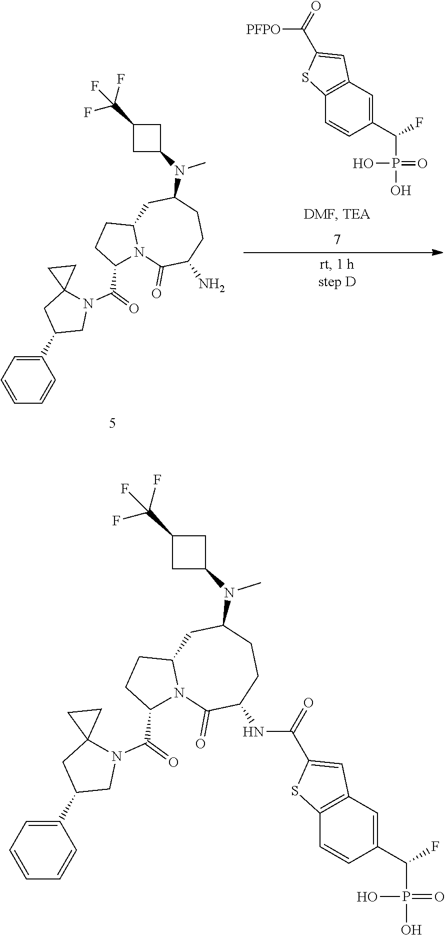

### Step A: tert-butyl ((3S,6S,9S,10aR)-5-oxo-3-((S)-6-phenyl-4-azaspiro[2.4]heptane-4-carbonyl)-9-(((1 s,3R)-3-(trifluoromethyl)cyclobutyl)amino)decahydropyrrolo[1,2-a]azocin-6-yl)carbamate

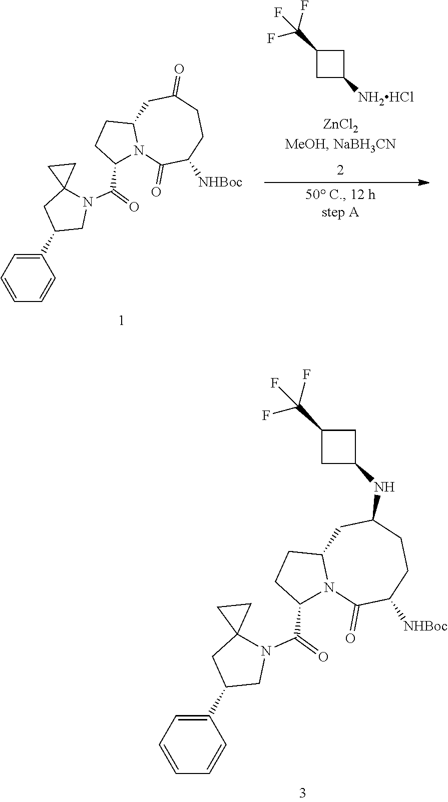

To a solution of tert-butyl ((3S,6S,10aR)-5,9-dioxo-3-((R)-6-phenyl-4-azaspiro[2.4]heptane-4-carbonyl)decahydropyrrolo[1,2-a]azocin-6-yl)carbamate (1, 1 g, 2.02 mmol, 1 eq.) in MeOH (20 mL) was added (1s,3s)-3-(trifluoromethyl)cyclobutan-1-amine hydrochloride (2, 530 mg, 3.03 mmol, 1.5 eq.), ZnCl₂ (2.77 g, 20.2 mmol, 10 eq.), and the solution was stirred at 50° C. for 2 hr under the atmosphere of N₂, then NaBH₃CN (1.27 g, 20.2 mmol, 10 eq.) added portion wise every 30 mins (2 eq. (254 mg each time) added 5 times). The solution was stirred at 50° C. for additional 12 hr, then cooled to room temperature and used to next step without further workup. LCMS (ESI): m/z=619 [M+H]⁺.

### Step B: tert-butyl ((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamate

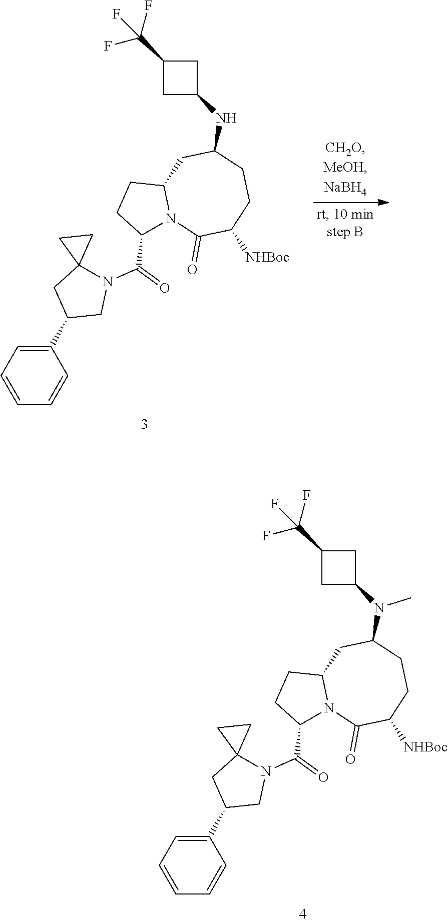 [SMILES: CC(C)(C)OC(=O)N[C@H]1CC[C@H](N[C@H]2C[C@@H](C(F)(F)F)C2)C[C@H]2CC[C@@H](C(=O)N3C[C@H](c4ccccc4)CC34CC4)N2C1=O ; CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](C(F)(F)F)C1]

To the above mixture (step A) was added 40% formaldehyde aqueous (3 mL) at room temperature, the mixture was stirred for 30 min, then NaBH₄ (383 mg, 10.1 mmol, 5 eq.) was added in several portions and the resulting mixture was stirred for additional 30 min. The resulting mixture was cooled down to 0° C. and was quenched by NH₄Cl (sat. aq), diluted with H₂O (80 mL) and extracted with EtOAc (50 mL×3), the organic layers were combined and washed with brine (50 mL), dried over Na₂SO₄, filtered, then concentrated to dryness under reduced pressure. The crude product was purified by C18 column (MeCN:H₂O (0.1% ammonium hydroxide)=5%-70%) to afford tert-butyl ((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamate (4, 1 g, 1.58 mmol, 78.9% yield two steps) as a white solid. LCMS (ESI): m/z=633 [M+H]⁺.

### Step C: (3S,6S,9S,10aR)-6-amino-9-(methyl((1s,3R)-3-(trifluoromethyl)cyclobutyl)amino)-3-((S)-6-phenyl-4-azaspiro[2.4]heptane-4-carbonyl)octahydropyrrolo[1,2-a]azocin-5(1H)-one

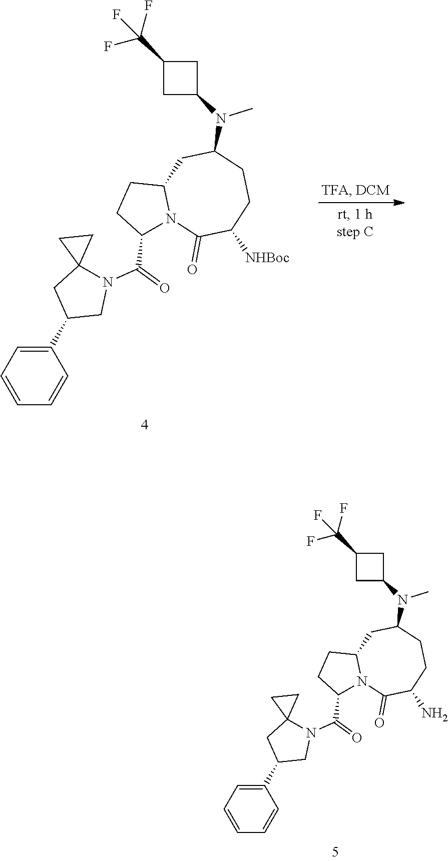 [SMILES: CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](C(F)(F)F)C1 ; CN([C@H]1CC[C@H](N)C(=O)N2[C@H](CC[C@H]2C(=O)N2C[C@H](c3ccccc3)CC23CC3)C1)[C@H]1C[C@@H](C(F)(F)F)C1]

To a solution of tert-butyl ((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamate (4, 30 mg, 0.047 mmol) in DCM (2 mL) was added TFA (2 mL). The resulting mixture was stirred for 1 h at room temperature and concentrated to dryness under reduced pressure to afford crude product (3S,6S,9S,10aR)-6-amino-9-(methyl((1s,3R)-3-(trifluoromethyl)cyclobutyl)amino)-3-((S)-6-phenyl-4-azaspiro[2.4]heptane-4-carbonyl)octahydropyrrolo[1,2-a]azocin-5(1H)-one (5, 30 mg, TFA salt) as a yellow oil which was used in the next step without further purification. LCMS (ESI): m/z=533 [M+H]⁺.

### Step D: ((R)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

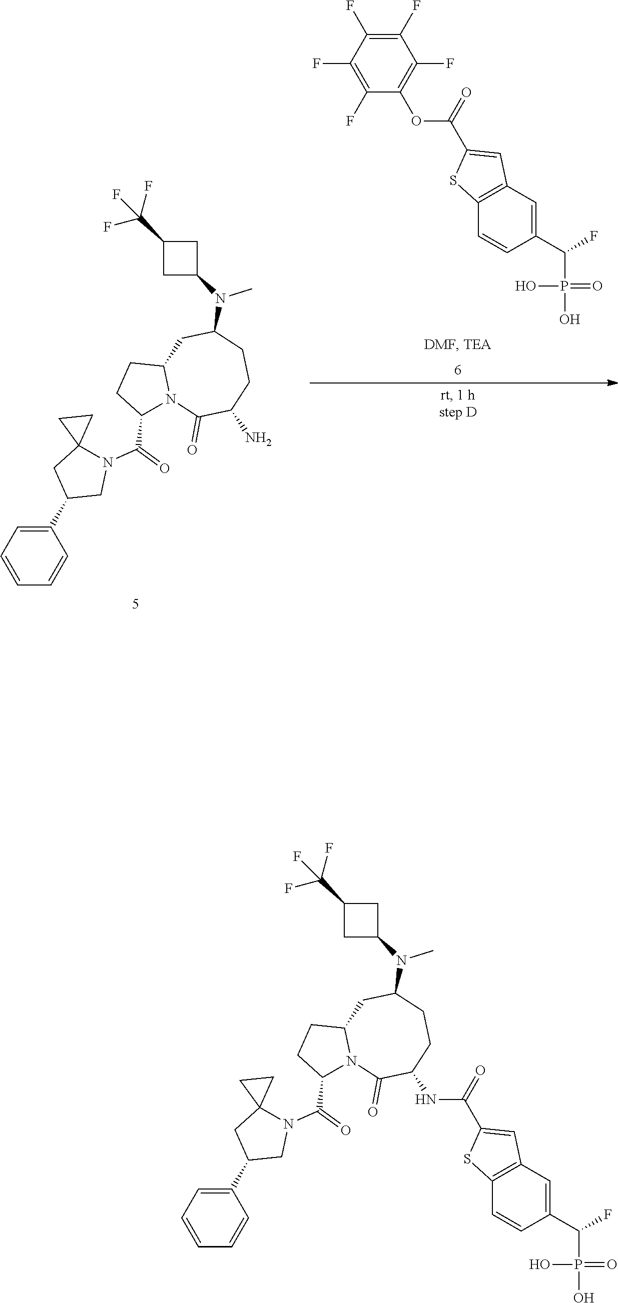 [SMILES: CN([C@H]1CC[C@H](N)C(=O)N2[C@H](CC[C@H]2C(=O)N2C[C@H](c3ccccc3)CC23CC3)C1)[C@H]1C[C@@H](C(F)(F)F)C1 ; O=C(Oc1c(F)c(F)c(F)c(F)c1F)c1cc2cc([C@H](F)P(=O)(O)O)ccc2s1 ; CN([C@H]1CC[C@H](NC(=O)c2cc3cc([C@H](F)P(=O)(O)O)ccc3s2)C(=O)N2[C@H](CC[C@H]2C(=O)N2C[C@H](c3ccccc3)CC23CC3)C1)[C@H]1C[C@@H](C(F)(F)F)C1]

To a solution of (3S,6S,9S,10aR)-6-amino-9-(methyl((1s,3R)-3-(trifluoromethyl)cyclobutyl)amino)-3-((S)-6-phenyl-4-azaspiro[2.4]heptane-4-carbonyl)octahydropyrrolo[1,2-a]azocin-5(1H)-one (5, 30 mg, TFA salt) in DMF (2 mL) was added TEA (9.6 mg, 0.095 mmol, 2 eq.) and (R)-(fluoro(2-((perfluorophenoxy)carbonyl)benzo[b]thiophen-5-yl)methyl)phosphonic acid (6, 21.6 mg, 0.047 mmol, 1 eq.). The resulting mixture was stirred for 1 h at room temperature and the crude product was purified by Prep-HPLC to afford ((R)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A2, 18 mg) as a white solid. LCMS (ESI): m/z=805 [M+H]⁺. ¹H NMR (400 MHz, CD₃OD-d₄) δ (ppm) 8.05 (s, 2H), 7.89 (d, J=8.5 Hz, 1H), 7.66 (d, J=8.4 Hz, 1H), 7.36-7.27 (m, 4H), 7.23-7.17 (m, 1H), 5.76-5.53 (m, 1H), 4.69-4.60 (m, 2H), 4.51-4.44 (m, 1H), 4.05-3.80 (m, 4H), 3.59-3.51 (m, 1H), 2.93-2.58 (m, 6H), 2.49-2.29 (m, 4H), 2.20-2.12 (m, 2H), 2.02-1.58 (m, 10H), 0.57-0.45 (m, 2H). ¹⁹F NMR (376 MHz, CD₃OD-d₄) δ (ppm) −74.51 (s), −195.88 (s). ³¹P NMR (162 MHz, CD₃OD-d₄) δ (ppm) 9.78 (d, J=74.9 Hz).

### Synthesis of ((S)-tetrahydrofuran-3-yl)methyl (((R)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C174)

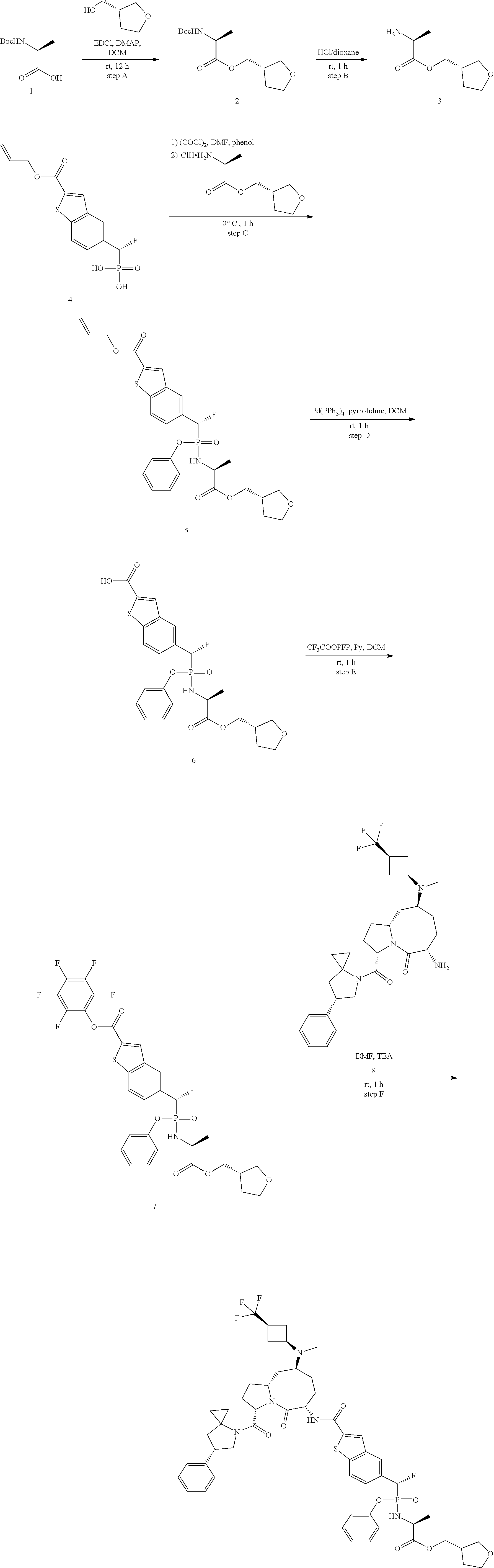

### Step A: ((S)-tetrahydrofuran-3-yl)methyl (tert-butoxycarbonyl)-L-alaninate

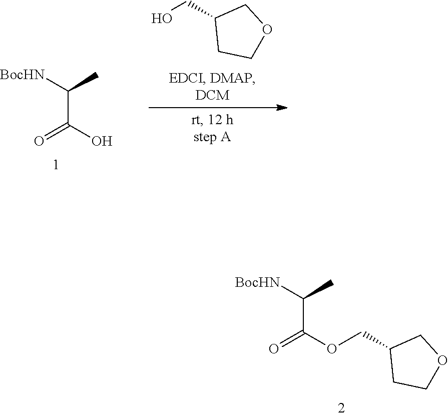 [SMILES: OC[C@H]1CCOC1 ; C[C@H](NC(=O)OC(C)(C)C)C(=O)O ; C[C@H](NC(=O)OC(C)(C)C)C(=O)OC[C@H]1CCOC1]

To a solution of (tert-butoxycarbonyl)-L-alanine (1, 2 g, 10.58 mmol, 1 eq.) in DCM (20 mL) was added (R)-(tetrahydrofuran-3-yl)methanol (1.08 g, 10.58 mmol, 1 eq.), DMAP (1.42 g, 11.64 mmol, 1.1 eq.) and EDCI (2.23 g, 11.64 mmol, 1.1 eq.). The solution was stirred for 12 h at room temperature and was quenched by adding H₂O (20 mL), then extracted with DCM (20 mL×2). The organic layers were combined and washed with brine (20 mL), dried over Na₂SO₄, filtered, and concentrated to dryness under reduced pressure. The crude product was purified by silica gel column to afford ((S)-tetrahydrofuran-3-yl)methyl (tert-butoxycarbonyl)-L-alaninate (2, 2.5 g, 9.16 mmol, 86.5% yield) as a colorless oil. LCMS (ESI): m/z=274 [M+H]⁺.

### Step B: ((S)-tetrahydrofuran-3-yl)methyl L-alaninate hydrochloride

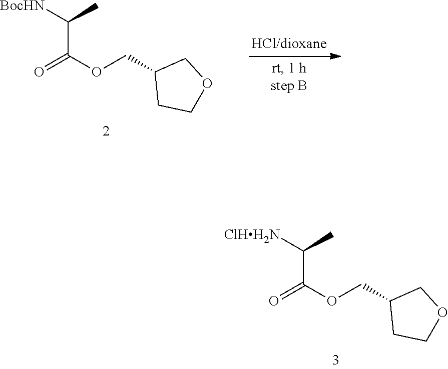

To a solution of ((S)-tetrahydrofuran-3-yl)methyl (tert-butoxycarbonyl)-L-alaninate (2, 2.5 g, 9.16 mmol) in dioxane (5 mL) was added 4M HCl in dioxane (10 mL). The solution was stirred for 1 hr at room temperature, LCMS showed that the reaction was completed. The solution was concentrated under vacuum to afford ((S)-tetrahydrofuran-3-yl)methyl L-alaninate hydrochloride (3, 1.9 g, 9.1 mmol, 99% yield) as a white solid (HCl salt). LCMS (ESI): m/z=174 [M+H]⁺.

### Step C: ((S)-tetrahydrofuran-3-yl)methyl L-alaninate hydrochloride

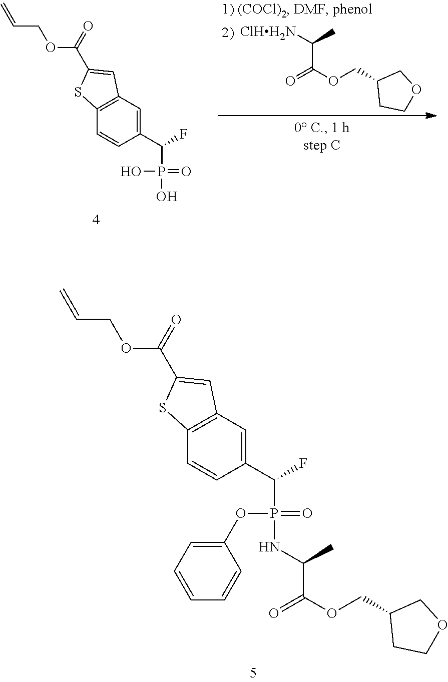

To a solution of (R)-((2-((allyloxy)carbonyl)benzo[b]thiophen-5-yl)fluoromethyl)phosphonic acid (4, 1.3 g, 3.94 mmol, 1 eq.) in DCM (20 mL) was added DMF (1 drop) and oxalyl chloride (2.5 g, 19.7 mmol, 5 eq). The resulting mixture was stirred for 1 h at room temperature, then excess oxalyl chloride was removed under reduced pressure. The residue was redissolved in DCM (20 mL) and cooled to 0° C., phenol (285 mg, 3.94 mmol, 1 eq) was added to the solution, TEA (2 g, 19.7 mmol, 5 eq.) was added dropwise, the mixture was stirred for 1 h at 0° C., ((S)-tetrahydrofuran-3-yl)methyl L-alaninate (1.27 g, 7.88 mmol, 2 eq.) was added, then the resulting mixture was stirred for additional 10 min. The reaction was quenched by adding H₂O (50 mL), then extracted with DCM (50 mL×2), the organic layers were combined and washed with brine, then dried over Na₂SO₄, filtered, and concentrated to dryness under reduced pressure. The crude product was purified by silica gel column chromatography to afford allyl 5-((1R)-fluoro((((S)-1-oxo-1-(((S)-tetrahydrofuran-3-yl)methoxy)propan-2-yl)amino)(phenoxy)phosphoryl)methyl)benzo[b]thiophene-2-carboxylate (5, 800 mg, 1.43 mmol, 47% yield) as a colorless oil. LCMS (ESI): m/z=562 [M+H]⁺.

### Step D: 5-((1R)-fluoro((((S)-1-oxo-1-(((S)-tetrahydrofuran-3-yl)methoxy)propan-2-yl)amino)(phenoxy)phosphoryl)methyl)benzo[b]thiophene-2-carboxylic acid

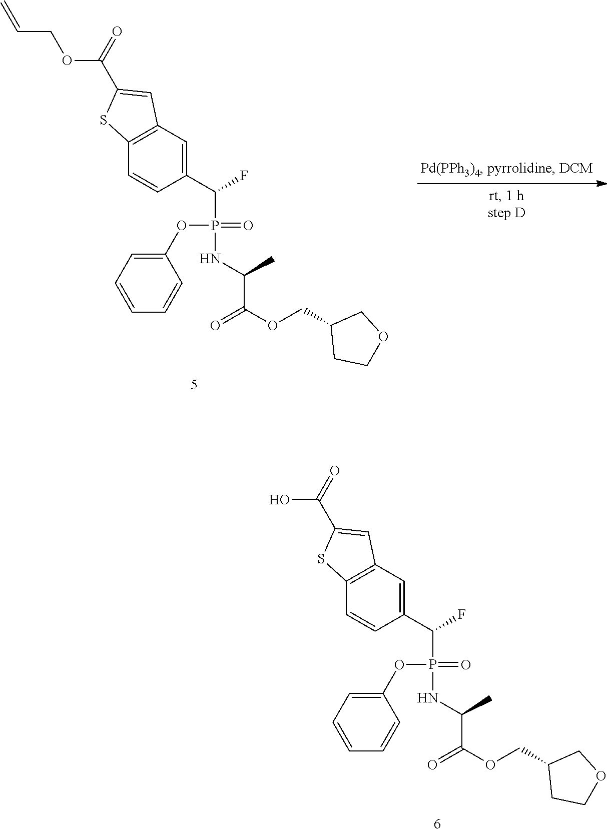 [SMILES: C=CCOC(=O)c1cc2cc([C@H](F)P(=O)(N[C@@H](C)C(=O)OC[C@H]3CCOC3)Oc3ccccc3)ccc2s1 ; C[C@H](NP(=O)(Oc1ccccc1)[C@@H](F)c1ccc2sc(C(=O)O)cc2c1)C(=O)OC[C@H]1CCOC1]

To a solution of allyl 5-((1R)-fluoro((((S)-1-oxo-1-(((S)-tetrahydrofuran-3-yl)methoxy)propan-2-yl)amino)(phenoxy)phosphoryl)methyl)benzo[b]thiophene-2-carboxylate (5, 800 mg, 1.43 mmol, 1 eq.) in DCM (10 mL) was added Pd(PPh₃)₄ (165 mg, 0.14 mmol, 0.1 eq.) and pyrrolidine (101 mg, 1.43 mmol, 1 eq.). The resulting mixture was stirred for 1 h at room temperature under the atmosphere of N₂. The pH was adjusted pH=3 with 1M HCl (aq.), then extracted with DCM (20 mL×2). The organic layers were combined and washed with brine (20 mL), dried over Na₂SO₄, filtered and the resulting mixture was concentrated to dryness under reduced pressure to afford crude product 5-((1R)-fluoro((((S)-1-oxo-1-(((S)-tetrahydrofuran-3-yl)methoxy)propan-2-yl)amino)(phenoxy)phosphoryl)methyl)benzo[b]thiophene-2-carboxylic acid (6, 700 mg, 1.34 mmol, 94% yield) as a yellow solid. LCMS (ESI): m/z=522 [M+H]⁺.

### Step E: perfluorophenyl 5-((1R)-fluoro((((S)-1-oxo-1-(((S)-tetrahydrofuran-3-yl)methoxy)propan-2-yl)amino)(phenoxy)phosphoryl)methyl)benzo[b]thiophene-2-carboxylate

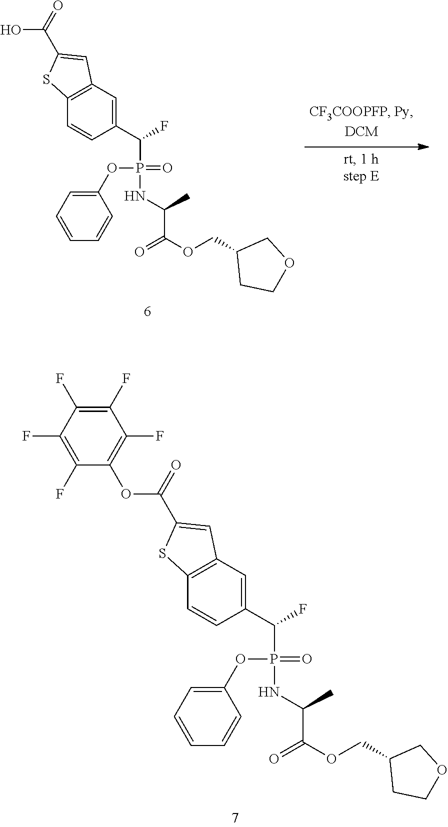 [SMILES: C[C@H](NP(=O)(Oc1ccccc1)[C@@H](F)c1ccc2sc(C(=O)O)cc2c1)C(=O)OC[C@H]1CCOC1 ; C[C@H](NP(=O)(Oc1ccccc1)[C@@H](F)c1ccc2sc(C(=O)Oc3c(F)c(F)c(F)c(F)c3F)cc2c1)C(=O)OC[C@H]1CCOC1]

To a solution of 5-((1R)-fluoro((((S)-1-oxo-1-(((S)-tetrahydrofuran-3-yl)methoxy)propan-2-yl)amino)(phenoxy)phosphoryl)methyl)benzo[b]thiophene-2-carboxylic acid (6, 700 mg, 1.34 mmol, 1 eq.) in DCM (10 mL) was added pyridine (318 mg, 4.03 mmol, 3 eq.) and perfluorophenyl 2,2,2-trifluoroacetate (753 mg, 2.69 mmol, 2 eq.). The resulting mixture was stirred for 1 h at room temperature, and the reaction was quenched by adding H₂O (20 mL), then extracted with DCM (20 mL×2). The organic layers were combined and washed with brine, dried over Na₂SO₄, concentrated to dryness under reduced pressure. The crude product was purified by silica gel column chromatography to afford perfluorophenyl 5-((1R)-fluoro((((S)-1-oxo-1-(((S)-tetrahydrofuran-3-yl)methoxy)propan-2-yl)amino)(phenoxy)phosphoryl)methyl)benzo[b]thiophene-2-carboxylate (7, 700 mg, 1.02 mmol, 76% yield) as a white solid. LCMS (ESI): m/z=688 [M+H]⁺.

### Step F: ((S)-tetrahydrofuran-3-yl)methyl (((R)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

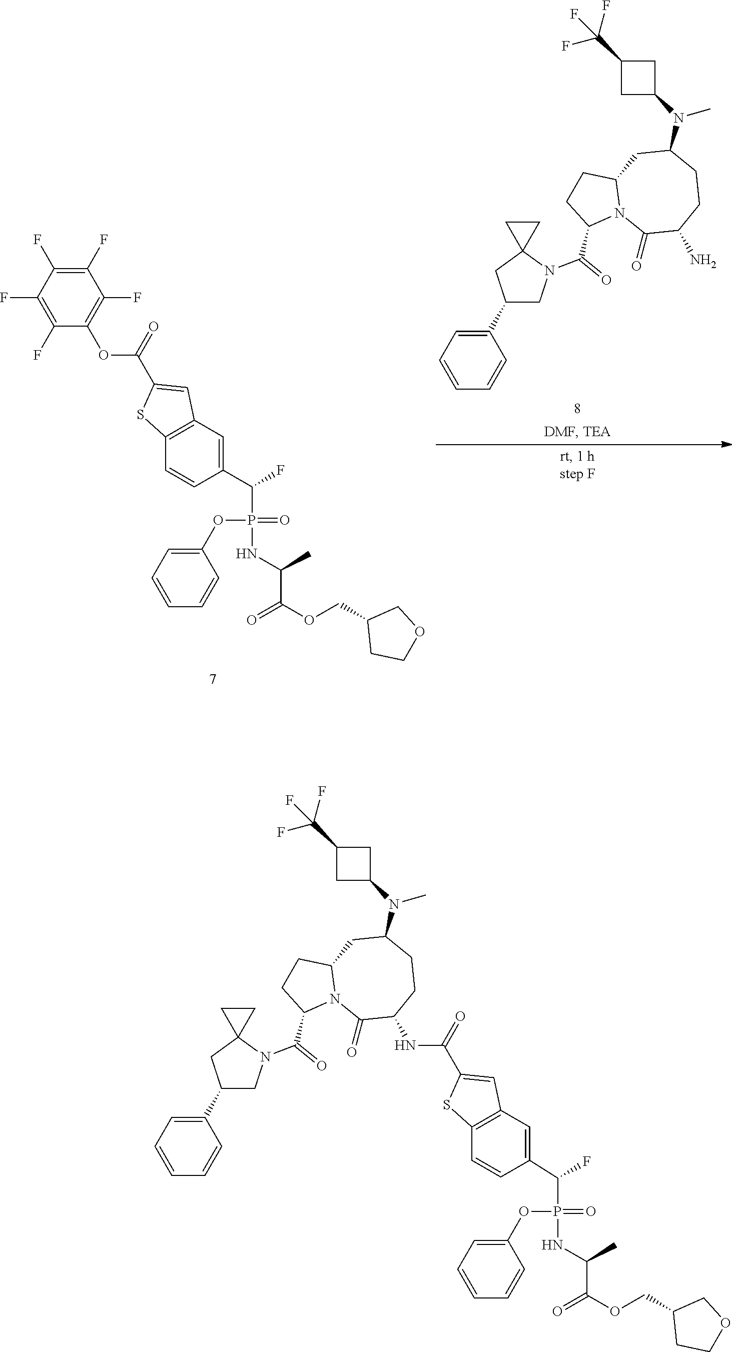 [SMILES: C[C@H](NP(=O)(Oc1ccccc1)[C@@H](F)c1ccc2sc(C(=O)Oc3c(F)c(F)c(F)c(F)c3F)cc2c1)C(=O)OC[C@H]1CCOC1 ; CN([C@H]1CC[C@H](N)C(=O)N2[C@H](CC[C@H]2C(=O)N2C[C@H](c3ccccc3)CC23CC3)C1)[C@H]1C[C@@H](C(F)(F)F)C1 ; C[C@H](NP(=O)(Oc1ccccc1)[C@@H](F)c1ccc2sc(C(=O)N[C@H]3CC[C@H](N(C)[C@H]4C[C@@H](C(F)(F)F)C4)C[C@H]4CC[C@@H](C(=O)N5C[C@H](c6ccccc6)CC56CC6)N4C3=O)cc2c1)C(=O)OC[C@H]1CCOC1]

To a solution of perfluorophenyl 5-((1R)-fluoro((((S)-1-oxo-1-(((S)-tetrahydrofuran-3-yl)methoxy)propan-2-yl)amino)(phenoxy)phosphoryl)methyl)benzo[b]thiophene-2-carboxylate (7, 20 mg, 0.029 mml, 1 eq.) in DMF (3 mL) was added (3S,6S,9S,10aR)-6-amino-9-(methyl((1s,3R)-3-(trifluoromethyl)cyclobutyl)amino)-3-((S)-6-phenyl-4-azaspiro[2.4]heptane-4-carbonyl)octahydropyrrolo[1,2-a]azocin-5(1H)-one (8, 16 mg, 0.029 mmol, 1 eq.), TEA (5.9 mg, 0.058 mmol, 2 eq). The resulting mixture was stirred for 1 hr at room temperature. The crude product was purified by Prep-HPLC to afford ((S)-tetrahydrofuran-3-yl)methyl (((R)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C174, 17 mg, 0.016 mmol) as a white solid. LCMS (ESI): m/z=1036 [M+H]⁺. ¹H NMR (400 MHz, DMSO-d₆) δ (ppm) 8.78 (d, J=7.1 Hz, 1H), 8.27 (s, 1H), 8.10-8.00 (m, 2H), 7.60 (d, J=8.3 Hz, 1H), 7.40-7.26 (m, 6H), 7.23-7.12 (m, 4H), 6.29-6.07 (m, 2H), 4.76-4.66 (m, 1H), 4.48 (t, J=8.4 Hz, 1H), 4.34-4.22 (m, 1H), 4.12 (t, J=8.6 Hz, 1H), 3.98-3.84 (m, 1H), 3.81-3.49 (m, 7H), 3.07-2.92 (m, 2H), 2.83-2.69 (m, 1H), 2.40-2.19 (m, 4H), 2.14-2.03 (m, 3H), 2.02-1.90 (m, 6H), 1.88-1.33 (m, 12H), 1.17-1.05 (m, 3H), 0.52-0.37 (m, 2H). ¹⁹F NMR (376 MHz, DMSO-d₆) δ (ppm) −71.25 (s), −194.09 (d, J=82.9 Hz), −196.24 (d, J=85.9 Hz). ³¹P NMR (162 MHz, DMSO-d₆) δ (ppm) 17.35 (d, J=82.7 Hz), 17.09 (d, J=85.8 Hz).

### Synthesis of ((R)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A3)

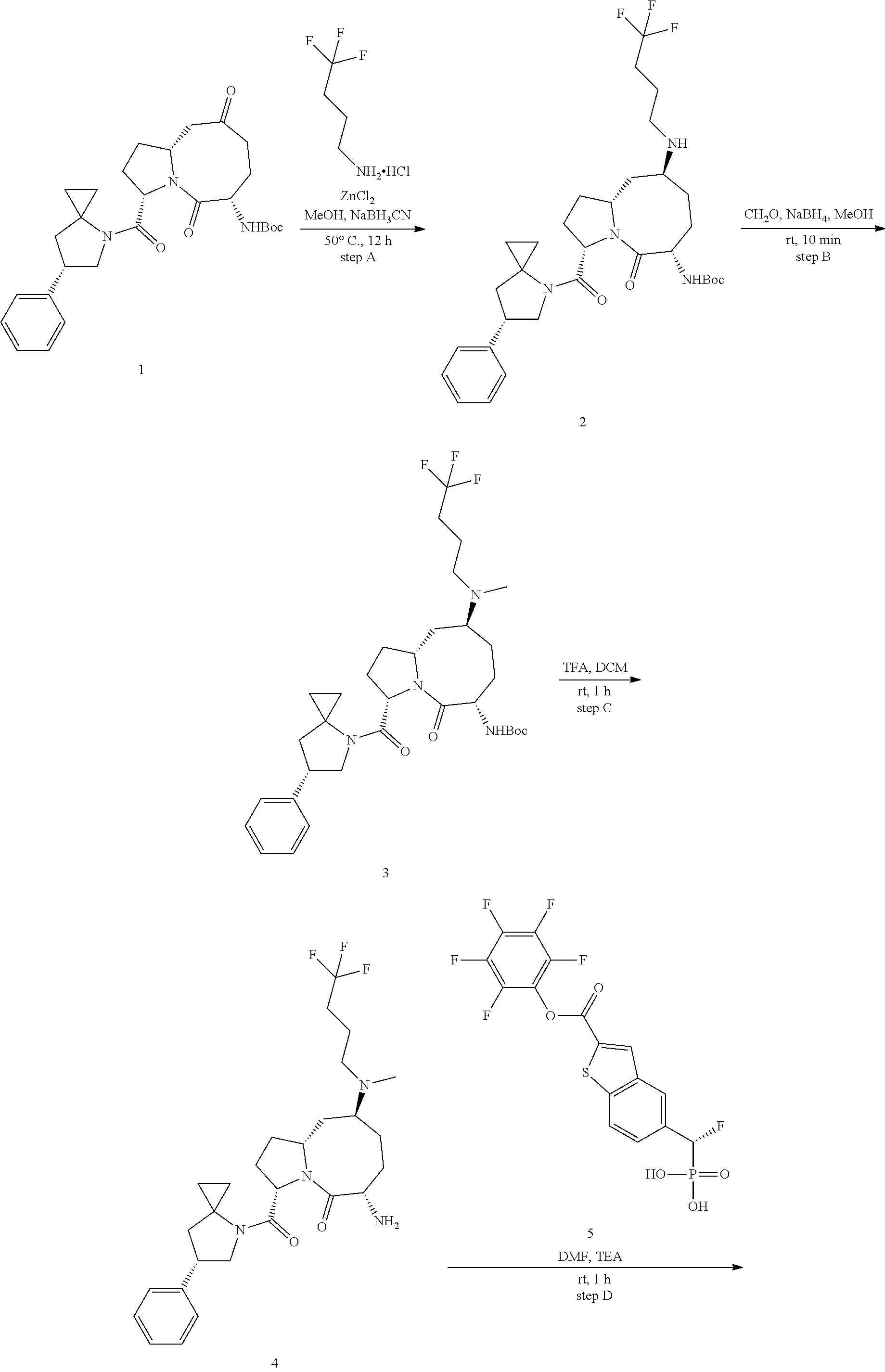

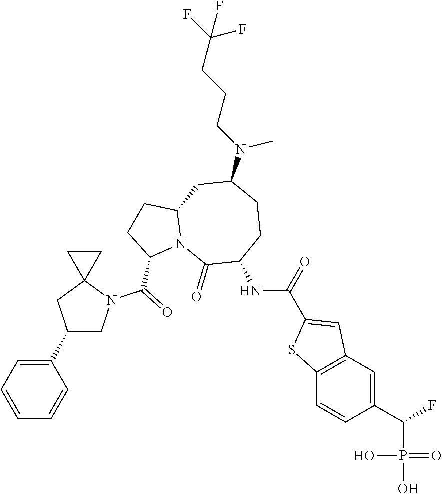 [SMILES: CN(CCCC(F)(F)F)[C@H]1CC[C@H](NC(=O)c2cc3cc([C@H](F)P(=O)(O)O)ccc3s2)C(=O)N2[C@H](CC[C@H]2C(=O)N2C[C@H](c3ccccc3)CC23CC3)C1]

### Step A: tert-butyl ((3S,6S,9S,10aR)-5-oxo-3-((S)-6-phenyl-4-azaspiro[2.4]heptane-4-carbonyl)-9-((4,4,4-trifluorobutyl)amino)decahydropyrrolo[1,2-a]azocin-6-yl)carbamate

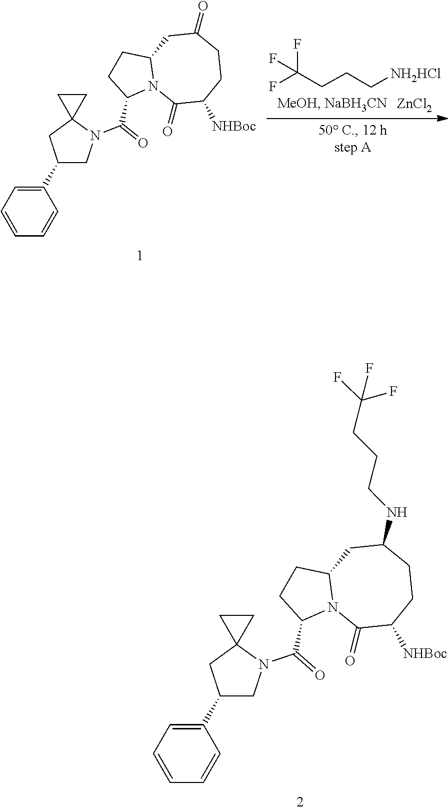

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (1, 2 g, 4.04 mmol, 1 eq.) in MeOH (40 mL) was added 4,4,4-trifluorobutan-1-amine hydrochloride (988 mg, 6.06 mml, 1.5 eq.), ZnCl₂ (5.5 g, 40.4 mmol, 10 eq.). The resulting mixture was stirred for 1 h at 50° C., then NaBH₃CN (2.54 g, 40.4 mmol, 10 eq.) was added portion wise every 30 mins (2 eq. (508 mg each time) added 5 times). The reaction mixture was stirred at 50° C. for additional 12 h, LCMS show that the reaction was completed. Then cooled to room temperature, the mixture was used to the next step directly without further workup. LCMS (ESI): m/z=607 [M+H]⁺.

### Step B: tert-butyl ((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamate

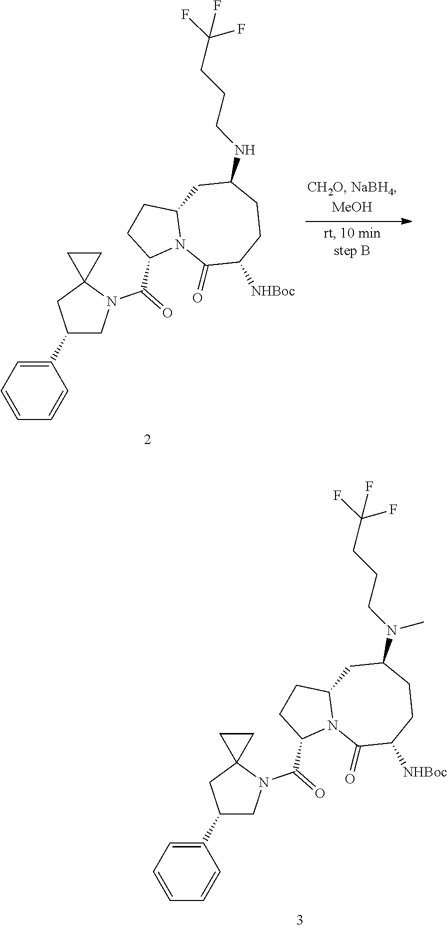 [SMILES: CC(C)(C)OC(=O)N[C@H]1CC[C@H](NCCCC(F)(F)F)C[C@H]2CC[C@@H](C(=O)N3C[C@H](c4ccccc4)CC34CC4)N2C1=O ; CN(CCCC(F)(F)F)[C@H]1CC[C@H](NC(=O)OC(C)(C)C)C(=O)N2[C@H](CC[C@H]2C(=O)N2C[C@H](c3ccccc3)CC23CC3)C1]

To the above mixture (step A) was added 40% formaldehyde (2 mL) at room temperature and the mixture was stirred for 10 min, then NaBH₄ (1.53 g, 40.4 mmol, 10 eq.) was added to the mixture, stirred for another 10 min. The reaction mixture was cooled down to 0° C. and quenched by NH₄Cl (sat. aq.), diluted with H₂O (100 mL) and extracted with EtOAc (50 mL×3). The organic layers were combined and washed with brine (20 mL), dried over Na₂SO₄, filtered, then concentrated to dryness under reduced pressure, the crude product was purified by C18 column (MeCN:H₂O (0.1% ammonium hydroxide)=5%-70%) to afford tert-butyl ((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamate (3, 1.5 g, 2.42 mmol, 80% yield, two steps) as a white solid. LCMS (ESI): m/z=621 [M+H]⁺.

### Step C: (3S,6S,9S,10aR)-6-amino-9-(methyl(4,4,4-trifluorobutyl)amino)-3-((S)-6-phenyl-4-azaspiro[2.4]heptane-4-carbonyl)octahydropyrrolo[1,2-a]azocin-5(1H)-one

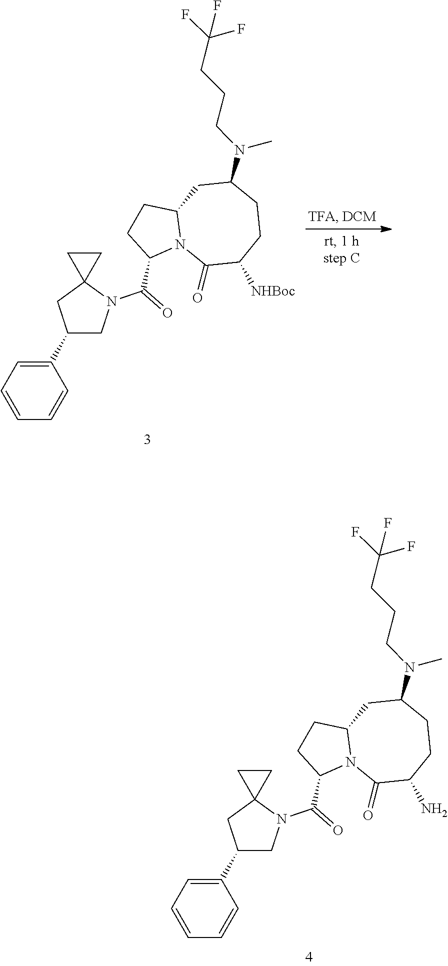 [SMILES: CN(CCCC(F)(F)F)[C@H]1CC[C@H](NC(=O)OC(C)(C)C)C(=O)N2[C@H](CC[C@H]2C(=O)N2C[C@H](c3ccccc3)CC23CC3)C1 ; CN(CCCC(F)(F)F)[C@H]1CC[C@H](N)C(=O)N2[C@H](CC[C@H]2C(=O)N2C[C@H](c3ccccc3)CC23CC3)C1]

To a solution of tert-butyl ((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamate (3, 20 mg, 0.032 mmol) in DCM (2 mL) was added TFA (2 mL), the resulting mixture was stirred for 1 h at room temperature then concentrated to dryness under reduced pressure to afford crude product (3S,6S,9S,10aR)-6-amino-9-(methyl(4,4,4-trifluorobutyl)amino)-3-((S)-6-phenyl-4-azaspiro[2.4]heptane-4-carbonyl)octahydropyrrolo[1,2-a]azocin-5(1H)-one (4, 20 mg, TFA salt) as a yellow oil which used in the next step without further purification. LCMS (ESI): m/z=521 [M+H]⁺.

### Step D: ((R)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

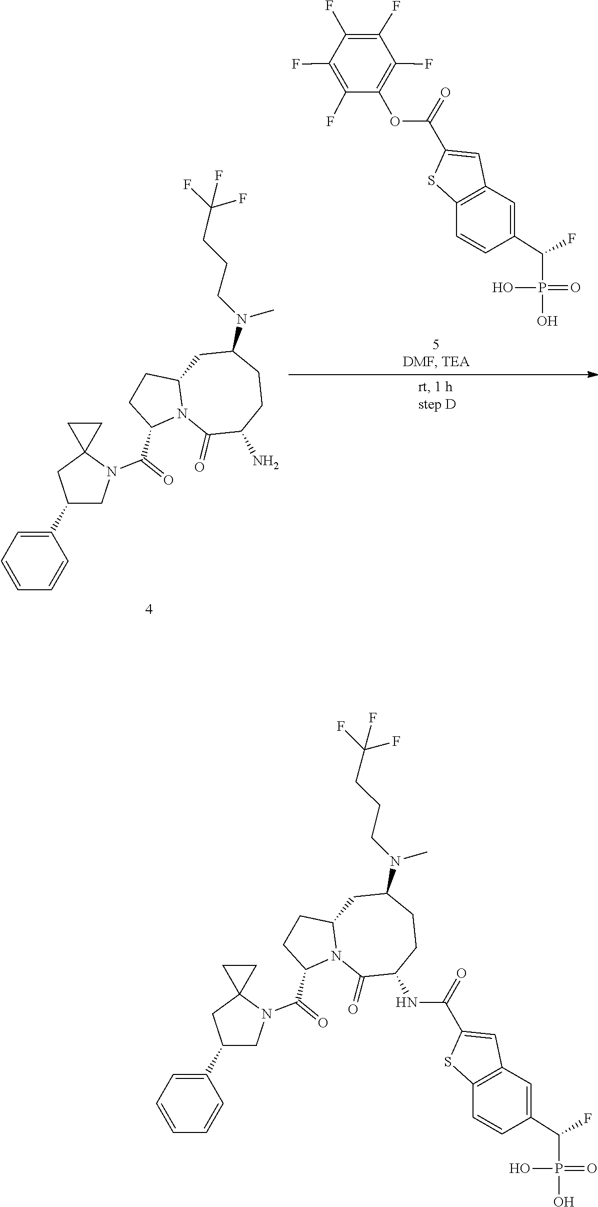 [SMILES: CN(CCCC(F)(F)F)[C@H]1CC[C@H](N)C(=O)N2[C@H](CC[C@H]2C(=O)N2C[C@H](c3ccccc3)CC23CC3)C1 ; O=C(Oc1c(F)c(F)c(F)c(F)c1F)c1cc2cc([C@H](F)P(=O)(O)O)ccc2s1 ; CN(CCCC(F)(F)F)[C@H]1CC[C@H](NC(=O)c2cc3cc([C@H](F)P(=O)(O)O)ccc3s2)C(=O)N2[C@H](CC[C@H]2C(=O)N2C[C@H](c3ccccc3)CC23CC3)C1]

To a solution of (3S,6S,9S,10aR)-6-amino-9-(methyl(4,4,4-trifluorobutyl)amino)-3-((S)-6-phenyl-4-azaspiro[12.4]heptane-4-carbonyl)octahydropyrrolo[1,2-a]azocin-5(1H)-one (4, 20 mg, TFA salt) in DMF (2 mL) was added TEA (6.5 mg, 0.065 mmol, 2 eq.), and (R)-(fluoro(2-((perfluorophenoxy)carbonyl)benzo[ib]thiophen-5-yl)methyl)phosphonic acid (6, 14.67 mg, 0.032 mmol, 1 eq). The resulting mixture was stirred for 1 h at room temperature. The crude product was purified by Prep-HPLC to afford ((R)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A3, 9 mg, 0.011 mmol) as a white solid. LCMS (ESI): m/z=793 [M+H]⁺. ¹H NMR (400 MHz, CD₃OD-d₄) δ (ppm) 8.09 (s, 1H), 8.05 (s, 1H), 7.94 (d, J=8.5 Hz, 1H), 7.70 (d, J=8.6 Hz, 1H), 7.38-7.26 (m, 4H), 7.22 (d, J=6.7 Hz, 1H), 5.79-5.58 (m, 1H), 4.64-4.47 (m, 2H), 4.38-4.21 (m, 1H), 4.07-3.80 (m, 2H), 3.77-3.63 (m, 1H), 3.58-3.47 (m, 1H), 3.14-2.94 (m, 2H), 2.73-2.60 (m, 3H), 2.53-1.47 (m, 19H), 0.60-0.36 (in, 2H). ¹⁹F NMR (377 MHz, CD₃OD-d₄) δ (ppm) −66.25˜−68.60 (m), −193.51˜−198.61 (m). ³¹P NMR (162 MHz, CD₃OD-d₄) δ (ppm) 9.79 (d, J=76.6 Hz).

### Synthesis of isobutyl ((S)-((R)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C135)

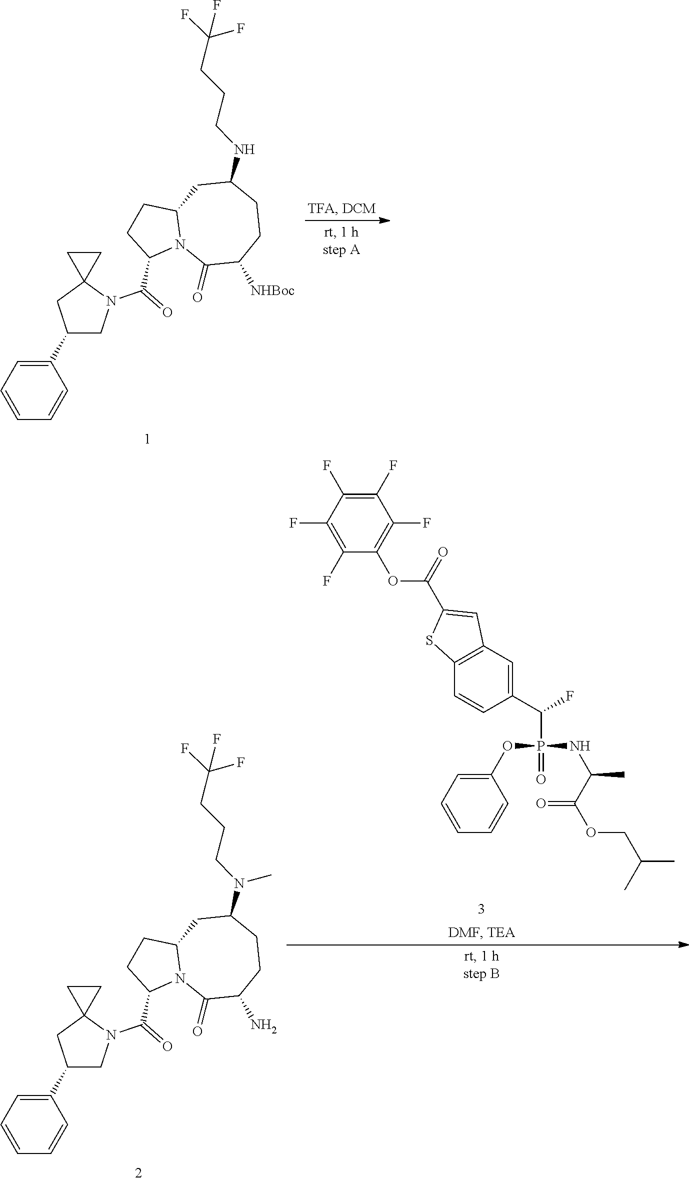 [SMILES: CC(C)(C)OC(=O)N[C@H]1CC[C@H](NCCCC(F)(F)F)C[C@H]2CC[C@@H](C(=O)N3C[C@H](c4ccccc4)CC34CC4)N2C1=O ; CN(CCCC(F)(F)F)[C@H]1CC[C@H](N)C(=O)N2[C@H](CC[C@H]2C(=O)N2C[C@H](c3ccccc3)CC23CC3)C1 ; CC(C)COC(=O)[C@H](C)N[P@@](=O)(Oc1ccccc1)[C@@H](F)c1ccc2sc(C(=O)Oc3c(F)c(F)c(F)c(F)c3F)cc2c1]

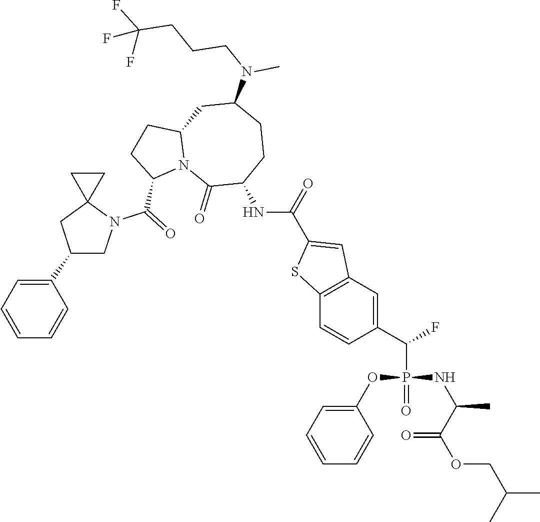 [SMILES: CC(C)COC(=O)[C@H](C)N[P@@](=O)(Oc1ccccc1)[C@@H](F)c1ccc2sc(C(=O)N[C@H]3CC[C@H](N(C)CCCC(F)(F)F)C[C@H]4CC[C@@H](C(=O)N5C[C@H](c6ccccc6)CC56CC6)N4C3=O)cc2c1]

### Step A: (3S,6S,9S,10aR)-6-amino-9-(methyl(4,4,4-trifluorobutyl)amino)-3-((S)-6-phenyl-4-azaspiro[2.4]heptane-4-carbonyl)octahydropyrrolo[1,2-a]azocin-5(1H)-one

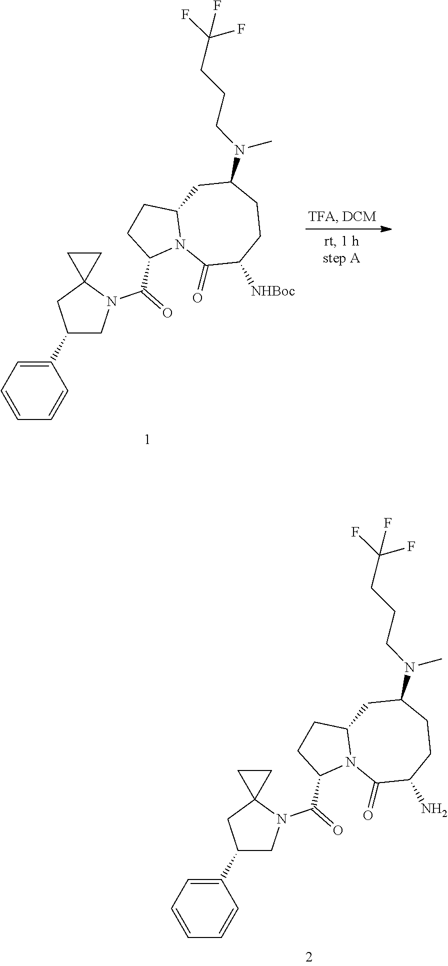 [SMILES: CN(CCCC(F)(F)F)[C@H]1CC[C@H](NC(=O)OC(C)(C)C)C(=O)N2[C@H](CC[C@H]2C(=O)N2C[C@H](c3ccccc3)CC23CC3)C1 ; CN(CCCC(F)(F)F)[C@H]1CC[C@H](N)C(=O)N2[C@H](CC[C@H]2C(=O)N2C[C@H](c3ccccc3)CC23CC3)C1]

To a solution of tert-butyl ((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamate (1, 20 mg, 0.032 mmol) in DCM (2 mL) was added TFA (2 mL) and the resulting mixture was stirred for 1 h at room temperature. The resulting mixture was concentrated to dryness under reduced pressure to afford crude product (3S,6S,9S,10aR)-6-amino-9-(methyl(4,4,4-trifluorobutyl)amino)-3-((S)-6-phenyl-4-azaspiro[2.4]heptane-4-carbonyl)octahydropyrrolo[1,2-a]azocin-5(1H)-one (2, 20 mg, TFA salt) as a yellow oil which used in the next step without further purification. LCMS (ESI): m/z=521 [M+H]⁺.

### Step B: isobutyl ((S)-((R)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

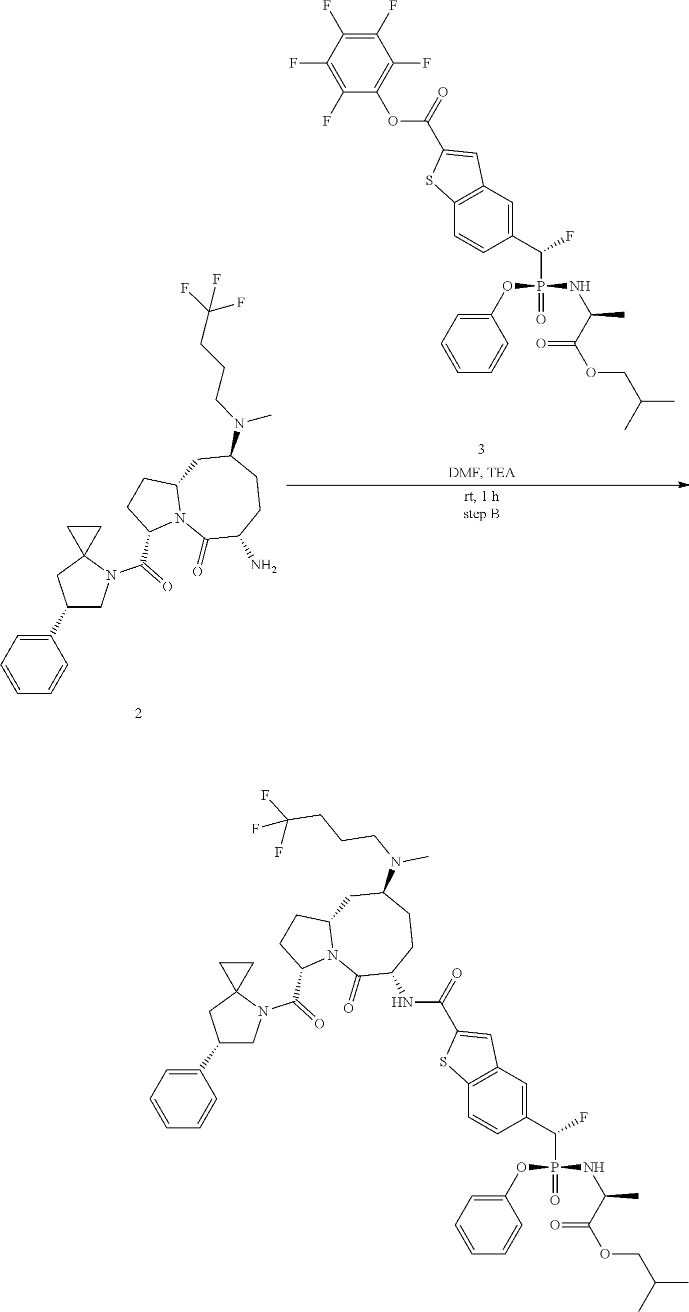 [SMILES: CN(CCCC(F)(F)F)[C@H]1CC[C@H](N)C(=O)N2[C@H](CC[C@H]2C(=O)N2C[C@H](c3ccccc3)CC23CC3)C1 ; CC(C)COC(=O)[C@H](C)N[P@@](=O)(Oc1ccccc1)[C@@H](F)c1ccc2sc(C(=O)Oc3c(F)c(F)c(F)c(F)c3F)cc2c1 ; CC(C)COC(=O)[C@H](C)N[P@@](=O)(Oc1ccccc1)[C@@H](F)c1ccc2sc(C(=O)N[C@H]3CC[C@H](N(C)CCCC(F)(F)F)C[C@H]4CC[C@@H](C(=O)N5C[C@H](c6ccccc6)CC56CC6)N4C3=O)cc2c1]

To a solution of (3S,6S,9S,10aR)-6-amino-9-(methyl(4,4,4-trifluorobutyl)amino)-3-((S)-6-phenyl-4-azaspiro[2.4]heptane-4-carbonyl)octahydropyrrolo[1,2-a]azocin-5(1H)-one (2, 20 mg, TFA salt) in DMF (2 mL) was added TEA (6.5 mg, 0.065 mmol, 2 eq.), and perfluorophenyl 5-((R)-fluoro((S)-(((S)-1-isobutoxy-1-oxopropan-2-yl)amino)(phenoxy)phosphoryl)methyl)benzo[b]thiophene-2-carboxylate (3, 21.2 mg, 0.032 mmol, 1 eq). The resulting mixture was stirred for 1 h at room temperature. The crude product was purified by Prep-HPLC to afford isobutyl ((S)-((R)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C135, 10 mg, 0.010 mmol) as a white solid. LCMS (ESI): m/z=996 [M+H]⁺. ¹H NMR (400 MHz, DMSO-d₆) δ (ppm) 8.85-8.67 (m, 1H), 8.34-8.18 (m, 1H), 8.12-7.99 (m, 2H), 7.68-7.53 (m, 1H), 7.39-7.14 (m, 10H), 6.32-6.07 (m, 2H), 4.89-4.69 (m, 1H), 4.56-4.40 (m, 1H), 4.36-4.21 (m, 1H), 4.16-4.04 (m, 1H), 3.85-3.67 (m, 4H), 3.58-3.44 (m, 1H), 3.01-2.85 (m, 1H), 2.46-2.17 (m, 7H), 2.14-2.08 (m, 3H), 2.05-1.95 (m, 3H), 1.87-1.52 (m, 11H), 1.19-1.07 (m, 3H), 0.89-0.75 (m, 6H), 0.54-0.37 (m, 2H). ¹⁹F NMR (376 MHz, DMSO-d₆) δ (ppm) −64.55 (s), −196.27 (d, J=85.8 Hz). ³¹P NMR (162 MHz, DMSO-d₆) δ (ppm) 17.08 (d, J=86.2 Hz). (free base).

### Synthesis of ((R)-(2-(((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A20)

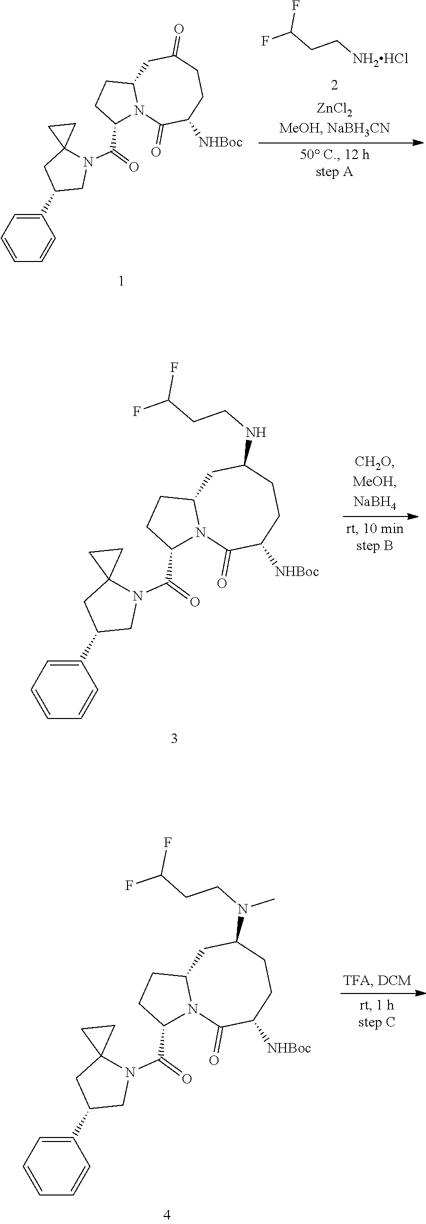

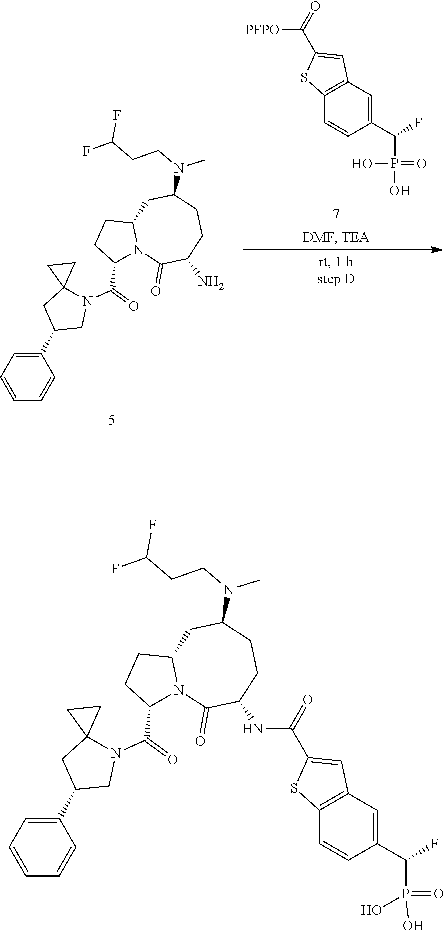

### Step A: tert-butyl ((3S,6S,9S,10aR)-9-((3,3-difluoropropyl)amino)-5-oxo-3-((R)-6-phenyl-4-azaspiro[2.4]heptane-4-carbonyl)decahydropyrrolo[1,2-a]azocin-6-yl)carbamate

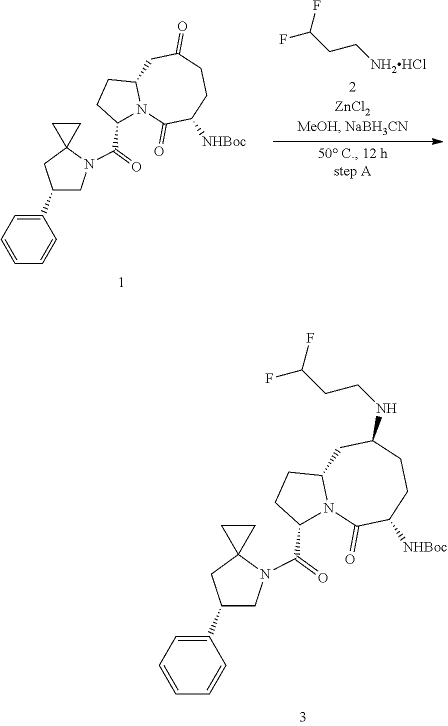

To a solution of tert-butyl ((3S,6S,10aR)-5,9-dioxo-3-((R)-6-phenyl-4-azaspiro[2.4]heptane-4-carbonyl)decahydropyrrolo[1,2-a]azocin-6-yl)carbamate (1, 1.5 g, 3.03 mmol, 1 eq.) in MeOH (30 mL) was added 3,3-difluoropropan-1-amine hydrochloride (2, 595 mg, 4.55 mmol, 1.5 eq.), ZnCl₂ (4.2 g, 30.3 mmol, 10 eq.) and the solution was stirred for 2 hr at 50° C. under the atmosphere of N₂. Then NaBH₃CN (1.90 g, 30.3 mmol, 10 eq.) was added portion wise every 30 mins (2 eq. (380 mg each time) added 5 times). The solution was stirred for 12 hr at 50° C., then cooled to room temperature and used in the next step without further workup. LCMS (ESI): m/z=575 [M+H]⁺.

### Step B: tert-butyl ((3S,6S,9S,10aR)-9-((3,3-difluoropropyl)(methyl)amino)-5-oxo-3-((R)-6-phenyl-4-azaspiro[2.4]heptane-4-carbonyl)decahydropyrrolo[1,2-a]azocin-6-yl)carbamate

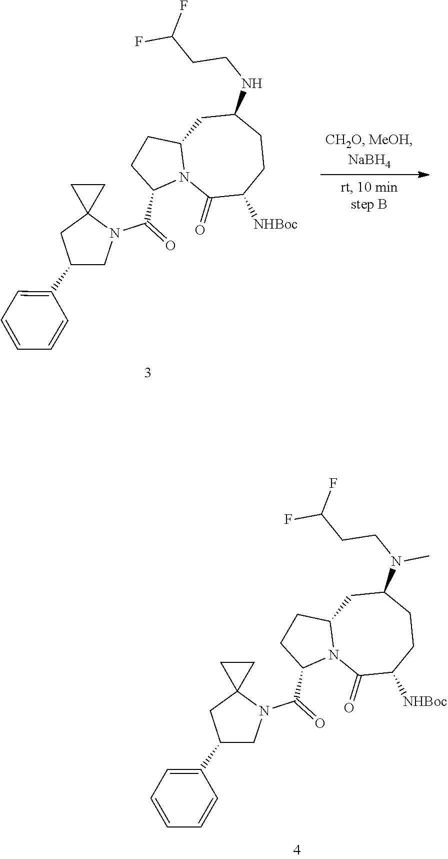 [SMILES: CC(C)(C)OC(=O)N[C@H]1CC[C@H](NCCC(F)F)C[C@H]2CC[C@@H](C(=O)N3C[C@H](c4ccccc4)CC34CC4)N2C1=O ; CN(CCC(F)F)[C@H]1CC[C@H](NC(=O)OC(C)(C)C)C(=O)N2[C@H](CC[C@H]2C(=O)N2C[C@H](c3ccccc3)CC23CC3)C1]

To the above mixture (step A) was added 40% formaldehyde aqueous (1.5 mL) at room temperature and the mixture was stirred for 10 min, then NaBH₄ (565 mg, 15.2 mmol, 5 eq.) was added in portions, and the mixture was stirred for additional 10 min. The reaction mixture was cooled down to 0° C., quenched by NH₄Cl (sat. aq), diluted with H₂O (100 mL), then extracted with EtOAc (50 mL×3), the organic layers were combined and washed with brine (20 mL), dried over Na₂SO₄, then concentrated to dryness under reduced pressure, and purified by C18 column (MeCN:H₂O (0.1% ammonium hydroxide)=5%-70%) to tert-butyl ((3S,6S,9S,10aR)-9-((3,3-difluoropropyl)(methyl)amino)-5-oxo-3-((R)-6-phenyl-4-azaspiro[2.4]heptane-4-carbonyl)decahydropyrrolo[1,2-a]azocin-6-yl)carbamate (4, 1.5 g, 2.55 mmol, 84% yield two steps) as a white solid. LCMS (ESI): m/z=589 [M+H]⁺.

### Step C: (3S,6S,9S,10aR)-6-amino-9-((3,3-difluoropropyl)(methyl)amino)-3-((R)-6-phenyl-4-azaspiro[2.4]heptane-4-carbonyl)octahydropyrrolo[1,2-a]azocin-5(1H)-one

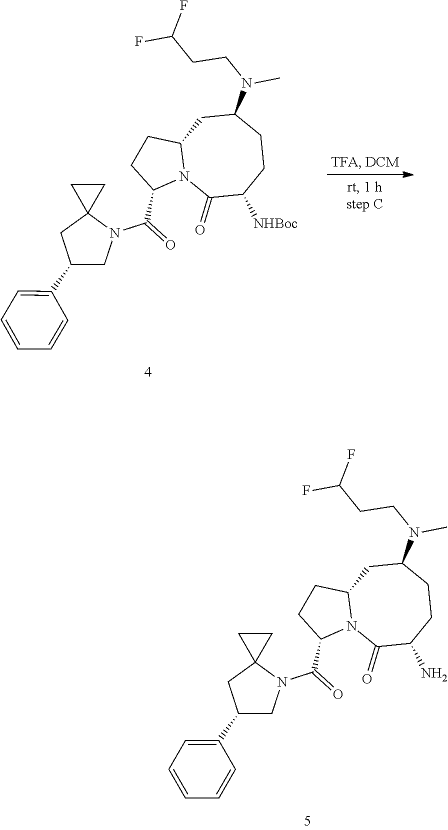 [SMILES: CN(CCC(F)F)[C@H]1CC[C@H](NC(=O)OC(C)(C)C)C(=O)N2[C@H](CC[C@H]2C(=O)N2C[C@H](c3ccccc3)CC23CC3)C1 ; CN(CCC(F)F)[C@H]1CC[C@H](N)C(=O)N2[C@H](CC[C@H]2C(=O)N2C[C@H](c3ccccc3)CC23CC3)C1]

To a solution of tert-butyl ((3S,6S,9S,10aR)-9-((3,3-difluoropropyl)(methyl)amino)-5-oxo-3-((R)-6-phenyl-4-azaspiro[2.4]heptane-4-carbonyl)decahydropyrrolo[1,2-a]azocin-6-yl)carbamate (4, 20 mg, 0.034 mmol) in DCM (2 mL) was added TFA (2 mL). The resulting mixture was stirred for 1 h at room temperature, and concentrated to dryness under reduced pressure to afford crude product (3S,6S,9S,10aR)-6-amino-9-((3,3-difluoropropyl)(methyl)amino)-3-((R)-6-phenyl-4-azaspiro[2.4]heptane-4-carbonyl)octahydropyrrolo[1,2-a]azocin-5(1H)-one (5, 20 mg, TFA salt) as a yellow oil which was used in the next step without further purification. LCMS (ESI): m/z=489 [M+H]⁺.

### Step D: ((R)-(2-(((3S,6S,9S,10aR)-9-((3,3-difluoropropyl)(methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid

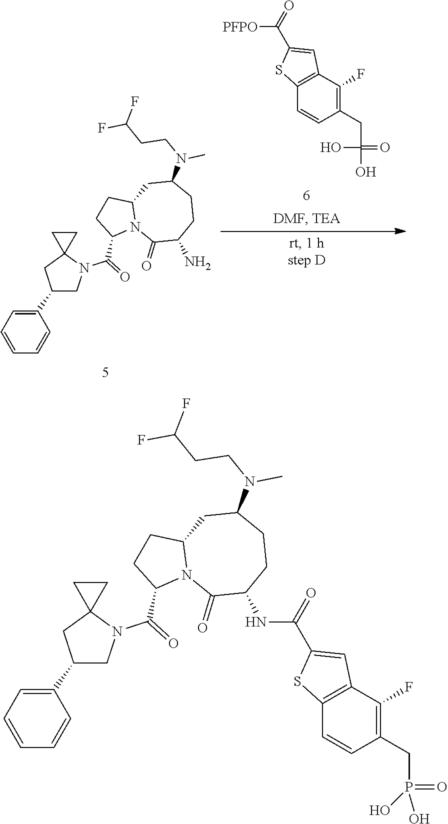

To a solution of (3S,6S,9S,10aR)-6-amino-9-((3,3-difluoropropyl)(methyl)amino)-3-((R)-6-phenyl-4-azaspiro[2.4]heptane-4-carbonyl)octahydropyrrolo[1,2-a]azocin-5(1H)-one (5, 20 mg, TFA salt) in DMF (2 mL) was added TEA (6.9 mg, 0.068 mmol, 2 eq.), (R)-(fluoro(2-((perfluorophenoxy)carbonyl)benzo[b]thiophen-5-yl)methyl)phosphonic acid (6, 15.5 mg, 0.034 mmol, 1 eq.). The resulting mixture was stirred for 1 hr at room temperature, LCMS show that the reaction was completed. The crude product was purified by Prep-HPLC to afford ((R)-(2-(((3S,6S,9S,10aR)-9-((3,3-difluoropropyl)(methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A20, 13 mg) as a white solid. LCMS (ESI): m/z=761 [M+H]⁺. 1H NMR (400 MHz, DMSO-d₆) δ (ppm) 8.87-8.67 (m, 1H), 8.24 (s, 1H), 8.03 (d, J=8.4 Hz, 1H), 7.98 (s, 1H), 7.53 (d, J=8.4 Hz, 1H), 7.38-7.29 (m, 4H), 7.26-7.19 (m, 1H), 6.40-6.03 (m, 1H), 5.91-5.75 (m, 1H), 4.81 (s, 1H), 4.60-4.50 (m, 2H), 4.17-4.09 (m, 1H), 3.85-3.70 (m, 2H), 3.57-3.51 (m, 1H), 3.35-3.19 (m, 2H), 2.80-2.69 (m, 3H), 2.49-2.42 (m, 1H), 2.39-1.70 (m, 14H), 1.56 (s, 1H), 0.59-0.42 (m, 2H). ¹⁹F NMR (377 MHz, DMSO-d₆) δ (ppm) −116.26 (d, J=78.4 Hz), −194.89 (d, J=80.9 Hz). ³¹P NMR (162 MHz, DMSO-d₆) δ (ppm) 10.94 (d, J=81.0 Hz).

### Synthesis of benzyl (((R)-(2-(((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C52)

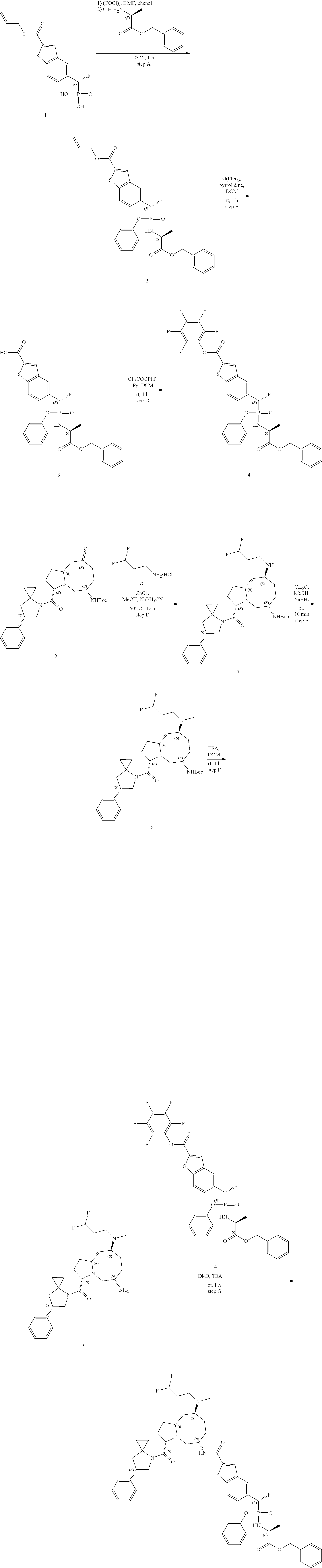

### Step A: allyl 5-((1R)—((((S)-1-(benzyloxy)-1-oxopropan-2-yl)amino)(phenoxy)phosphoryl)fluoromethyl)benzo[b]thiophene-2-carboxylate

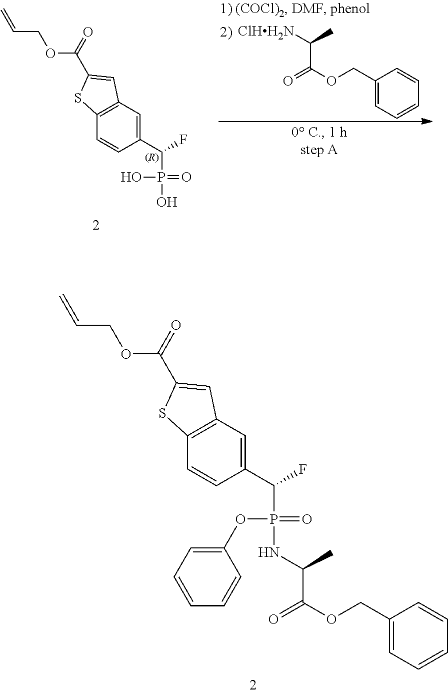

To a solution of (R)-((2-((allyloxy)carbonyl)benzo[b]thiophen-5-yl)fluoromethyl)phosphonic acid (1, 1.3 g, 3.94 mmol, 1 eq.) in DCM (20 mL) was added DMF (1 drop), oxalyl chloride (2.5 g, 19.7 mmol, 5 eq). The resulting mixture was stirred for 1 h at room temperature, then excess oxalyl chloride was removed under reduced pressure. The residue was redissolved in DCM (20 mL) and cooled to 0° C., phenol (285 mg, 3.94 mmol, 1 eq) was added to the solution, TEA (2 g, 19.7 mmol, 5 eq.) was added dropwise to the solution at 0° C., the mixture was stirred for 1 h at 0° C., benzyl L-alaninate hydrochloride (1.69 g, 7.88 mmol, 2 eq.) was added to the solution, then stirred for additional 10 min. The resulting mixture was quenched by adding H₂O (50 mL), then extracted with DCM (50 mL×2), and the organic layers were combined and washed with brine, then dried over Na₂SO₄, filtered, and concentrated to dryness under reduced pressure. The crude product was purified by silica gel column chromatography to afford allyl 5-((1R)—((((S)-1-(benzyloxy)-1-oxopropan-2-yl)amino)(phenoxy)phosphoryl)fluoromethyl)benzo[b]thiophene-2-carboxylate (2, 800 mg, 1.41 mmol, 36% yield) as a colorless oil. LCMS (ESI): m/z=568 [M+H]⁺.

### Step B: 5-((1R)-fluoro((((S)-1-oxo-1-(((S)-tetrahydrofuran-3-yl)methoxy)propan-2-yl)amino)(phenoxy)phosphoryl)methyl)benzo[b]thiophene-2-carboxylic acid

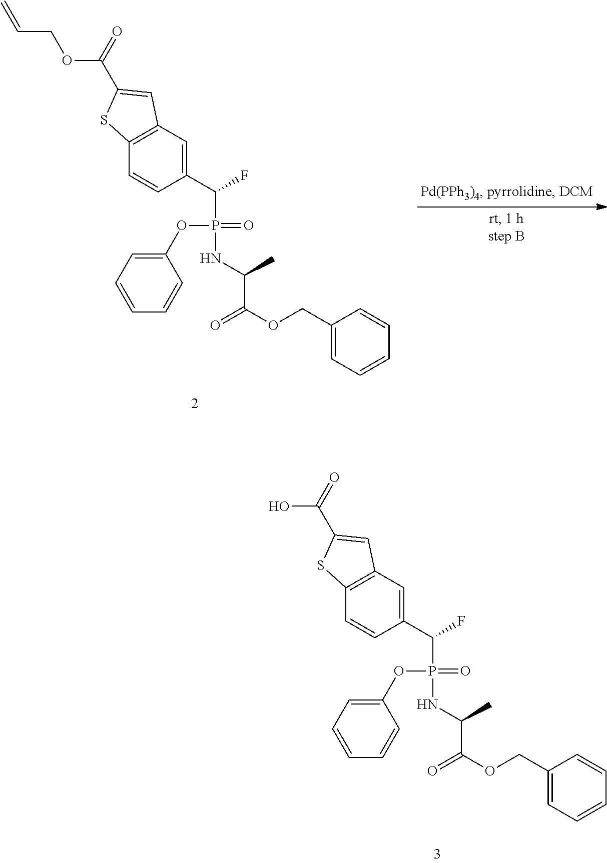 [SMILES: C=CCOC(=O)c1cc2cc([C@H](F)P(=O)(N[C@@H](C)C(=O)OCc3ccccc3)Oc3ccccc3)ccc2s1 ; C[C@H](NP(=O)(Oc1ccccc1)[C@@H](F)c1ccc2sc(C(=O)O)cc2c1)C(=O)OCc1ccccc1]

To a solution of allyl 5-((1R)—((((S)-1-(benzyloxy)-1-oxopropan-2-yl)amino)(phenoxy)phosphoryl)fluoromethyl)benzo[b]thiophene-2-carboxylate (2, 800 mg, 1.41 mmol, 1 eq.) in DCM (10 mL) was added Pd(PPh₃)₄ (165 mg, 0.14 mmol, 0.1 eq.) and pyrrolidine (100 mg, 1.41 mmol, 1 eq.). The resulting mixture was stirred for 1 h at room temperature under the atmosphere of N₂, LCMS show that the reaction was completed, adjusted pH=3 with 1M HCl (aq.), then extracted with DCM (20 mL×2). The organic layers were combined and washed with brine (20 mL) dried over Na₂SO₄, filtered, and concentrated to dryness under reduced pressure to afford crude product 5-((1R)—((((S)-1-(benzyloxy)-1-oxopropan-2-yl)amino)(phenoxy)phosphoryl)fluoromethyl)benzo[b]thiophene-2-carboxylic acid (3, 700 mg, 1.32 mmol, 94% yield) as a yellow solid. LCMS (ESI): m/z=528 [M+H]⁺.

### Step C: perfluorophenyl 5-((1R)—((((S)-1-(benzyloxy)-1-oxopropan-2-yl)amino)(phenoxy)phosphoryl)fluoromethyl)benzo[b]thiophene-2-carboxylate

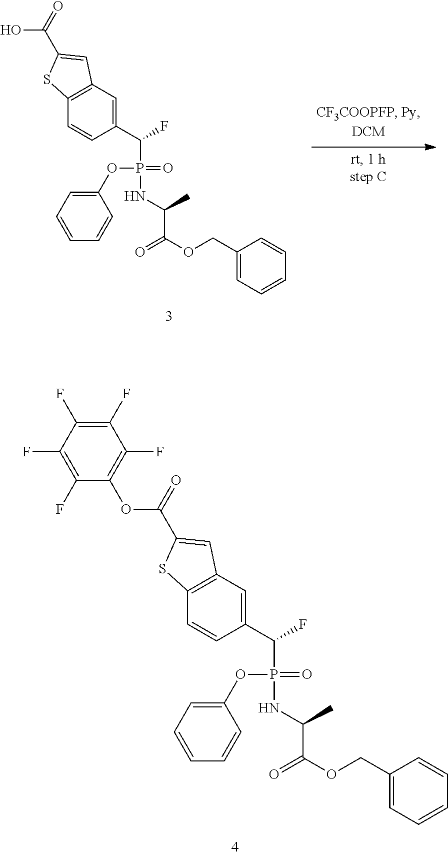 [SMILES: C[C@H](NP(=O)(Oc1ccccc1)[C@@H](F)c1ccc2sc(C(=O)O)cc2c1)C(=O)OCc1ccccc1 ; C[C@H](NP(=O)(Oc1ccccc1)[C@@H](F)c1ccc2sc(C(=O)Oc3c(F)c(F)c(F)c(F)c3F)cc2c1)C(=O)OCc1ccccc1]

To a solution of 5-((1R)—((((S)-1-(benzyloxy)-1-oxopropan-2-yl)amino)(phenoxy)phosphoryl)fluoromethyl)benzo[b]thiophene-2-carboxylic acid (3, 700 mg, 1.32 mmol, 1 eq.) in DCM (10 mL) was added pyridine (313 mg, 3.96 mmol, 3 eq.), perfluorophenyl 2,2,2-trifluoroacetate (739 mg, 2.64 mmol, 2 eq.). The resulting mixture was stirred for 1 h at room temperature and was quenched by adding H₂O (20 mL), then extracted with DCM (20 mL×2), the organic layers were combined and washed with brine, dried over Na₂SO₄, filtered, then concentrated under vacuum. The crude product was purified by silica gel column chromatography to afford perfluorophenyl 5-((1R)—((((S)-1-(benzyloxy)-1-oxopropan-2-yl)amino)(phenoxy)phosphoryl)fluoromethyl)benzo[b]thiophene-2-carboxylate (4, 700 mg, 1.01 mmol, 76% yield) as a white solid. LCMS (ESI): m/z=694 [M+H]⁺.

### Step D: tert-butyl ((3S,6S,9S,10aR)-9-((3,3-difluoropropyl)amino)-5-oxo-3-((S)-6-phenyl-4-azaspiro[2.4]heptane-4-carbonyl)decahydropyrrolo[1,2-a]azocin-6-yl)carbamate

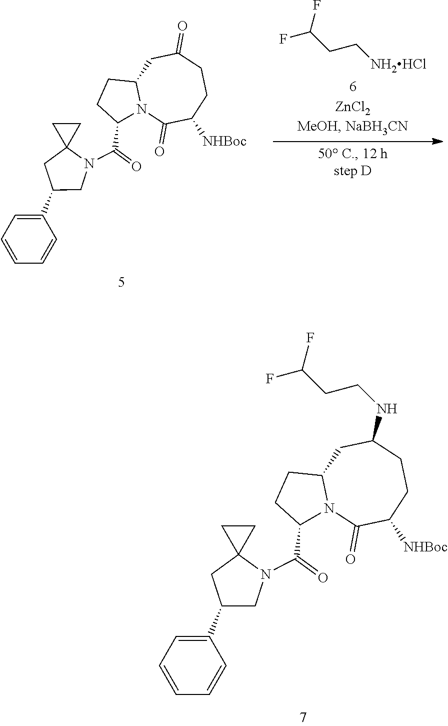

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (5, 3 g, 6.06 mmol, 1 eq.) in MeOH (60 mL) was added 3,3-difluoropropan-1-amine hydrochloride (2, 1.19 g, 9.09 mmol, 1.5 eq.), and ZnCl₂ (8.3 g, 60.6 mmol, 10 eq.) and the resulting solution was stirred for 2 hr at 50° C. under the atmosphere of N₂. Then NaBH₃CN (3.81 g, 60.6 mmol, 10 eq.) was added portion wise every 30 mins (2 eq. (762 mg each time) added 5 times). The solution was stirred at 50° C. for additional 12 hr, then cooled to room temperature and used in the next step without further purification. LCMS (ESI): m/z=575 [M+H]⁺.

### Step E: tert-butyl ((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamate

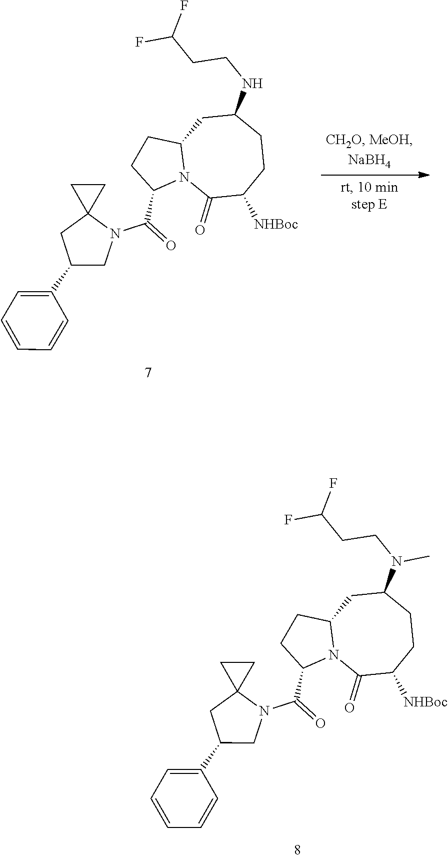 [SMILES: CC(C)(C)OC(=O)N[C@H]1CC[C@H](NCCC(F)F)C[C@H]2CC[C@@H](C(=O)N3C[C@H](c4ccccc4)CC34CC4)N2C1=O ; CN(CCC(F)F)[C@H]1CC[C@H](NC(=O)OC(C)(C)C)C(=O)N2[C@H](CC[C@H]2C(=O)N2C[C@H](c3ccccc3)CC23CC3)C1]

To the above mixture (step A) was added 40% formaldehyde aqueous (3 mL) at room temperature, the resulting mixture was stirred for 30 min, then NaBH₄ (1.15 g, 30.3 mmol, 5 eq.) was added in portions, and the mixture was stirred for additional 30 min. The reaction mixture was cooled down to 0° C., quenched by NH₄Cl (sat. aq), diluted with H₂O (200 mL), then extracted with EtOAc (100 mL×3), and the combined organic layers were washed with brine (20 mL), dried over Na₂SO₄, then concentrated to dryness under reduced pressure, the crude product was purified by C18 column (MeCN:H₂O (0.1% ammonium hydroxide)=5%-70%) to afford tert-butyl ((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamate (8, 3 g, 5.10 mmol, 84% yield two steps) as a white solid. LCMS (ESI): m/z=589 [M+H]⁺.

### Step F: (3S,6S,9S,10aR)-6-amino-9-((3,3-difluoropropyl)(methyl)amino)-3-((S)-6-phenyl-4-azaspiro[2.4]heptane-4-carbonyl)octahydropyrrolo[1,2-a]azocin-5(1H)-one

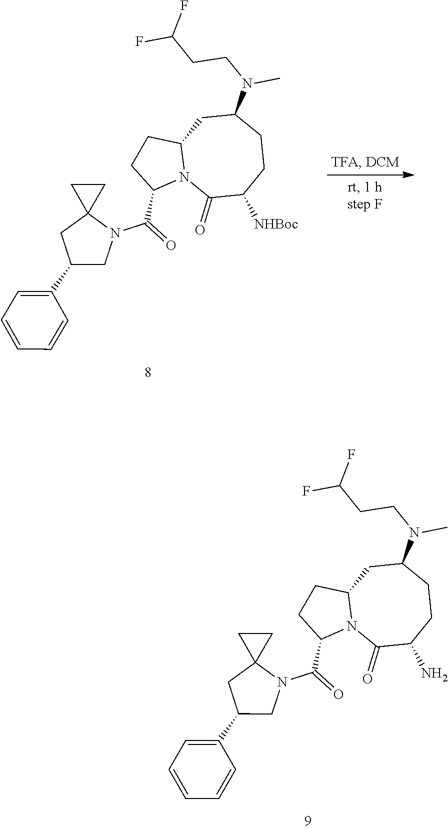 [SMILES: CN(CCC(F)F)[C@H]1CC[C@H](NC(=O)OC(C)(C)C)C(=O)N2[C@H](CC[C@H]2C(=O)N2C[C@H](c3ccccc3)CC23CC3)C1 ; CN(CCC(F)F)[C@H]1CC[C@H](N)C(=O)N2[C@H](CC[C@H]2C(=O)N2C[C@H](c3ccccc3)CC23CC3)C1]

To a solution of tert-butyl ((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamate (8, 20 mg, 0.034 mmol) in DCM (2 mL) was added TFA (2 mL). The resulting mixture was stirred for 1 h at room temperature. The resulting mixture was concentrated to dryness under reduced pressure to afford crude product (3S,6S,9S,10aR)-6-amino-9-((3,3-difluoropropyl)(methyl)amino)-3-((S)-6-phenyl-4-azaspiro[2.4]heptane-4-carbonyl)octahydropyrrolo[1,2-a]azocin-5(1H)-one (9, 20 mg, TFA salt) as a yellow oil which was used to the next step without further purification. LCMS (ESI): m/z=489 [M+H]⁺.

### Step G: benzyl (((R)-(2-(((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate

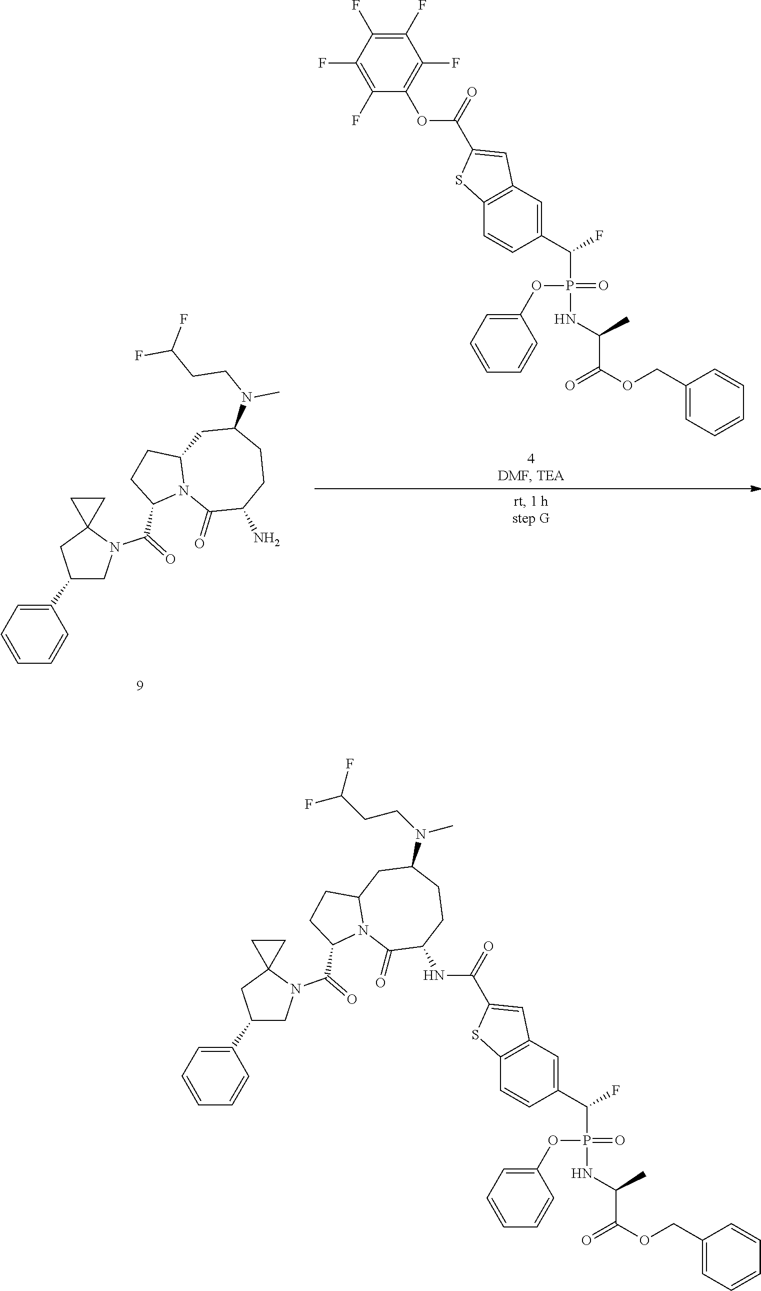 [SMILES: CN(CCC(F)F)[C@H]1CC[C@H](N)C(=O)N2[C@H](CC[C@H]2C(=O)N2C[C@H](c3ccccc3)CC23CC3)C1 ; C[C@H](NP(=O)(Oc1ccccc1)[C@@H](F)c1ccc2sc(C(=O)Oc3c(F)c(F)c(F)c(F)c3F)cc2c1)C(=O)OCc1ccccc1 ; C[C@H](NP(=O)(Oc1ccccc1)[C@@H](F)c1ccc2sc(C(=O)N[C@H]3CC[C@H](N(C)CCC(F)F)CC4CC[C@@H](C(=O)N5C[C@H](c6ccccc6)CC56CC6)N4C3=O)cc2c1)C(=O)OCc1ccccc1]

To a solution of (3S,6S,9S,10aR)-6-amino-9-((3,3-difluoropropyl)(methyl)amino)-3-((S)-6-phenyl-4-azaspiro[2.4]heptane-4-carbonyl)octahydropyrrolo[1,2-a]azocin-5(1H)-one (9, 20 mg, TFA salt) in DMF (2 mL) was added TEA (6.9 mg, 0.068 mmol, 2 eq.), perfluorophenyl 5-((1R)—((((S)-1-(benzyloxy)-1-oxopropan-2-yl)amino)(phenoxy)phosphoryl)fluoromethyl)benzo[b]thiophene-2-carboxylate (4, 23.6 mg, 0.034 mmol, 1 eq.). The resulting mixture was stirred for 1 hr at room temperature and the crude product was purified by Prep-HPLC to afford benzyl (((R)-(2-(((3S,6S,9S,10aR)-9-((3,3-difluoropropyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C52, 21 mg) as a white solid. LCMS (ESI): m/z=998 [M+H]⁺. ¹H NMR (400 MHz, DMSO-d₆) δ (ppm) 8.79 (M, 1H), 8.28 (s, 1H), 8.10-8.02 (m, 2H), 7.60 (t, J=7.5 Hz, 1H), 7.39-7.11 (m, 16H), 6.37-6.05 (m, 3H), 5.09-4.90 (m, 2H), 4.84-4.72 (m, 1H), 4.50 (t, J=8.5 Hz, 1H), 4.38-4.27 (m, 1H), 4.16-4.06 (m, 1H), 3.97-3.83 (m, 1H), 3.75 (t, J=9.7 Hz, 1H), 3.60-3.49 (m, 1H), 3.06-2.94 (m, 1H), 2.47-2.33 (m, 2H), 2.25-2.19 (m, 1H), 2.16-2.11 (m, 4H), 2.07-1.93 (m, 5H), 1.89-1.54 (m, 8H), 1.22-1.15 (m, 3H), 0.55-0.41 (m, 2H). ¹⁹F NMR (377 MHz, DMSO-d₆) δ (ppm) −115.74-−116.43 (m), −194.66-−196.64 (m). ³¹P NMR (162 MHz, DMSO-d₆) δ (ppm) 17.26 (d, J=82.3 Hz), 17.15 (d, J=86.2 Hz).

### Synthesis of ((R)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A21)

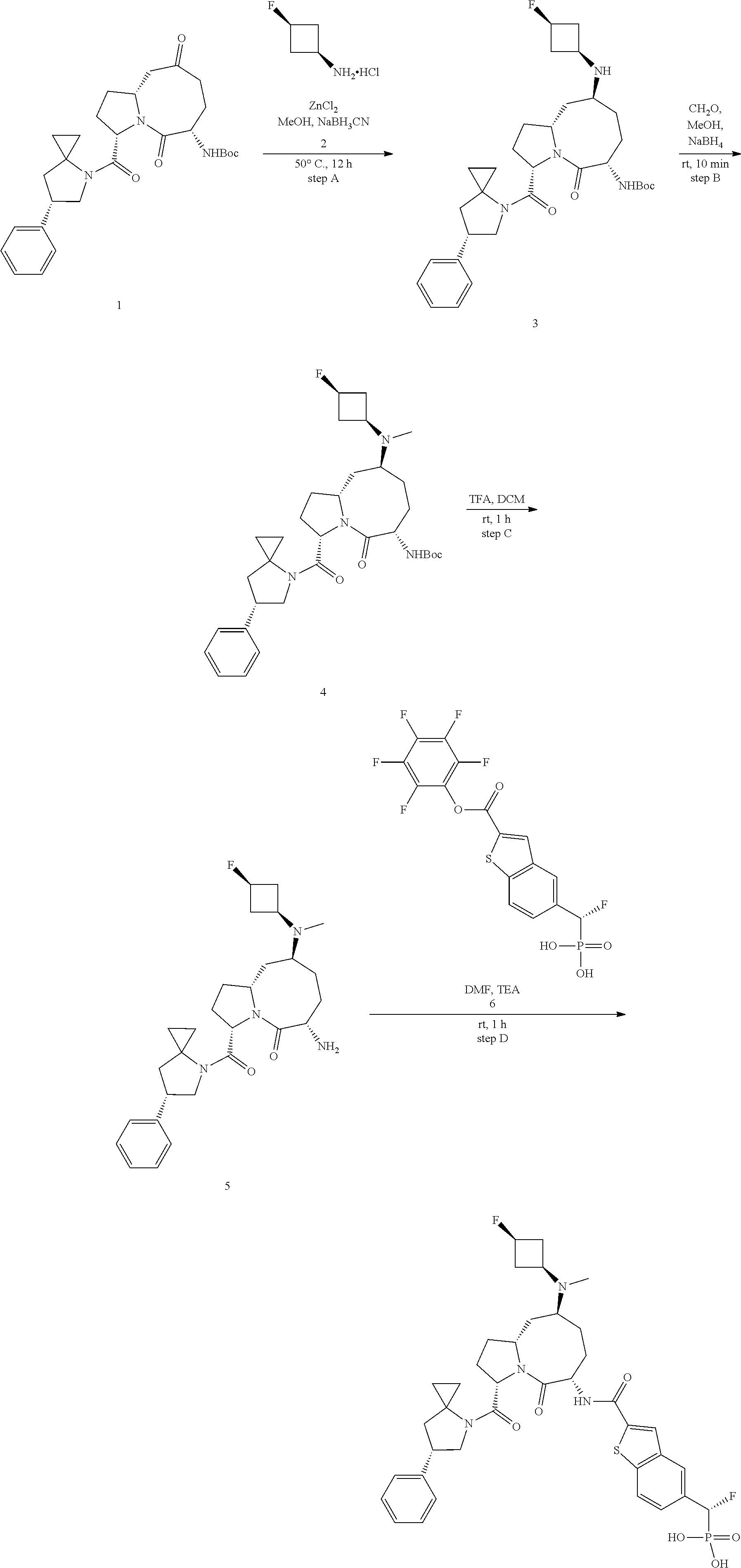

### Step A: tert-butyl ((3S,6S,9S,10aR)-9-(((1s,3R)-3-fluorocyclobutyl)amino)-5-oxo-3-((S)-6-phenyl-4-azaspiro[2.4]heptane-4-carbonyl)decahydropyrrolo[1,2-a]azocin-6-yl)carbamate

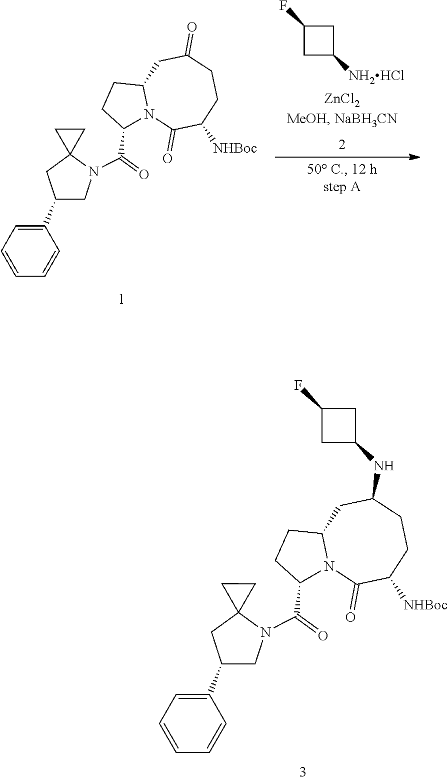

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (1, 3 g, 6.06 mmol, 1 eq.) in MeOH (60 mL) was added (1s,3s)-3-fluorocyclobutan-1-amine hydrochloride (2, 1.14 g, 9.09 mmol, 1.5 eq.), ZnCl₂ (8.3 g, 60.6 mmol, 10 eq.), the resulting mixture was stirred for 2 hr. at 50° C. under the atmosphere of N₂. Then NaBH₃CN (3.81 g, 60.6 mmol, 10 eq.) was added portion wise every 30 mins (2 eq. (762 mg each time) added 5 times). The solution was stirred for 12 hr at 50° C., then cooled to room temperature. The solution was used to next step without further workup. LCMS (ESI): m/z=569 [M+H]⁺.

### Step B: tert-butyl ((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamate

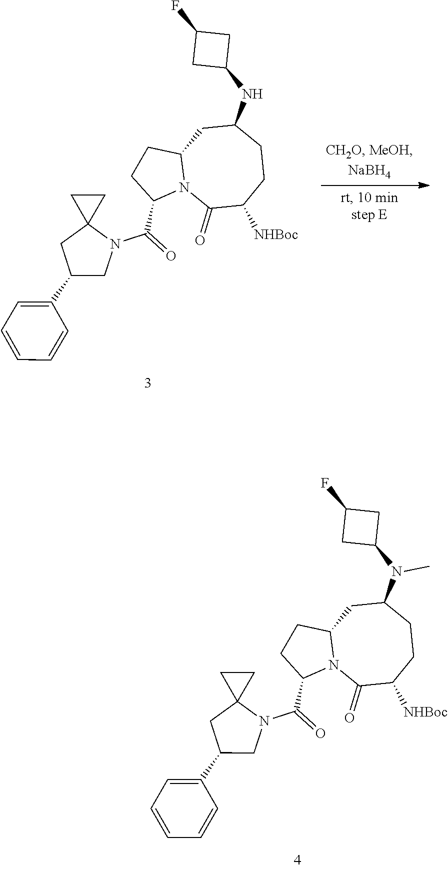 [SMILES: CC(C)(C)OC(=O)N[C@H]1CC[C@H](N[C@H]2C[C@@H](F)C2)C[C@H]2CC[C@@H](C(=O)N3C[C@H](c4ccccc4)CC34CC4)N2C1=O ; CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](F)C1]

To the above mixture (step A) was added 40% formaldehyde aqueous (3 mL) at room temperature, the mixture was stirred for 30 min, then NaBH₄ (1.15 g, 30.3 mmol, 5 eq.) was added in portions and the mixture was stirred for another 30 min. The reaction was quenched by NH₄Cl (sat. aq), diluted with H₂O (200 mL), then extracted with EtOAc (100 mL×3), the organic layers were combined and washed with brine (20 mL), dried over Na₂SO₄, filtered, then concentrated to dryness under reduced pressure. The crude product was purified by C18 column (MeCN:H₂O (0.1% ammonium hydroxide)=5%-70%) to afford tert-butyl ((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamate (4, 3 g, 5.15 mmol, 85% yield two steps) as a white solid. LCMS (ESI): m/z=583 [M+H]⁺.

### Step C: (3S,6S,9S,10aR)-6-amino-9-(((1s,3R)-3-fluorocyclobutyl)(methyl)amino)-3-((S)-6-phenyl-4-azaspiro[2.4]heptane-4-carbonyl)octahydropyrrolo[1,2-a]azocin-5(1H)-one

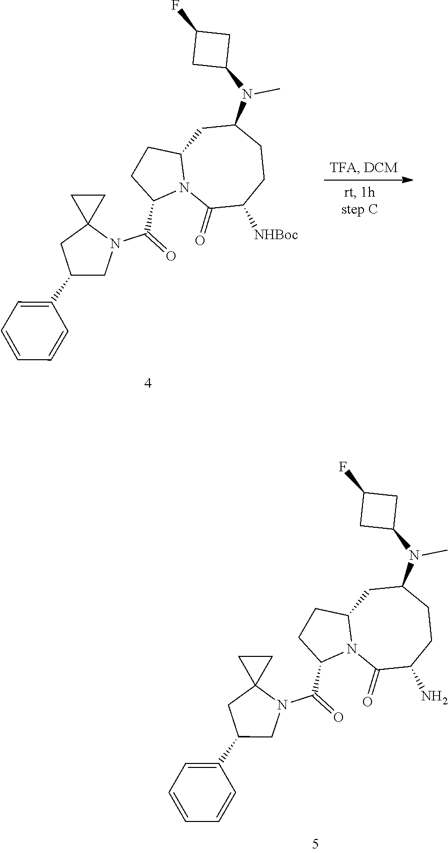 [SMILES: CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](F)C1 ; CN([C@H]1CC[C@H](N)C(=O)N2[C@H](CC[C@H]2C(=O)N2C[C@H](c3ccccc3)CC23CC3)C1)[C@H]1C[C@@H](F)C1]

To a solution of tert-butyl ((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamate (4, 20 mg, 0.034 mmol) in DCM (2 mL) was added TFA (2 mL). The resulting mixture was stirred for 1 h at room temperature. The resulting mixture was concentrated to dryness under reduced pressure to afford crude product (3S,6S,9S,10aR)-6-amino-9-(((1s,3R)-3-fluorocyclobutyl)(methyl)amino)-3-((S)-6-phenyl-4-azaspiro[2.4]heptane-4-carbonyl)octahydropyrrolo[1,2-a]azocin-5(1H)-one (5, 20 mg, TFA salt) as a yellow oil which was used to the next step without further purification. LCMS (ESI): m/z=483 [M+H]⁺.

### Step D: ((R)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

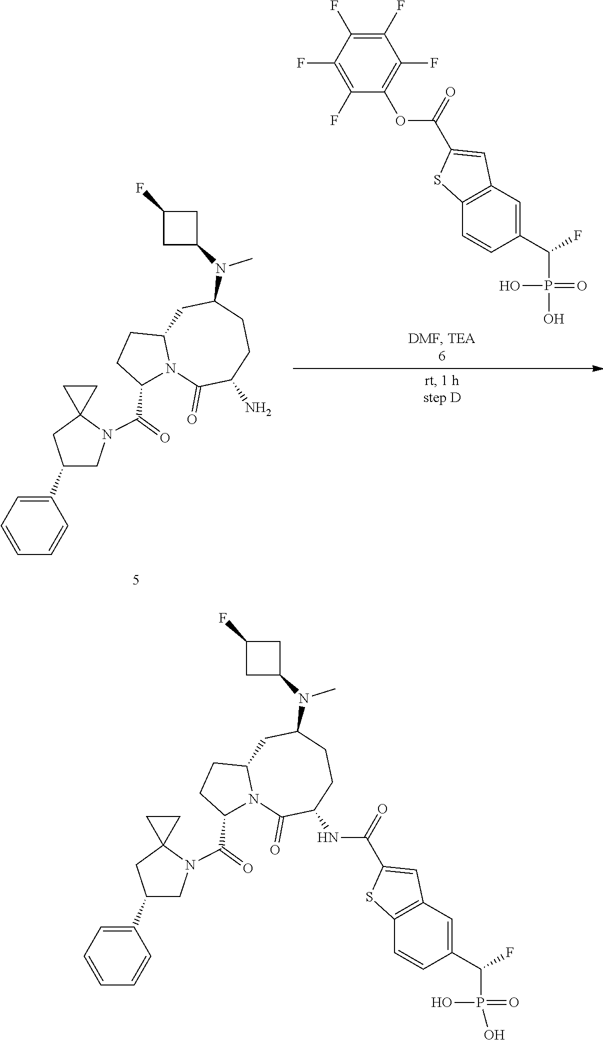 [SMILES: CN([C@H]1CC[C@H](N)C(=O)N2[C@H](CC[C@H]2C(=O)N2C[C@H](c3ccccc3)CC23CC3)C1)[C@H]1C[C@@H](F)C1 ; O=C(Oc1c(F)c(F)c(F)c(F)c1F)c1cc2cc([C@H](F)P(=O)(O)O)ccc2s1 ; CN([C@H]1CC[C@H](NC(=O)c2cc3cc([C@H](F)P(=O)(O)O)ccc3s2)C(=O)N2[C@H](CC[C@H]2C(=O)N2C[C@H](c3ccccc3)CC23CC3)C1)[C@H]1C[C@@H](F)C1]

To a solution of (3S,6S,9S,10aR)-6-amino-9-(((1s,3R)-3-fluorocyclobutyl)(methyl)amino)-3-((S)-6-phenyl-4-azaspiro[2.4]heptane-4-carbonyl)octahydropyrrolo[1,2-a]azocin-5(1H)-one (5, 20 mg, TFA salt) in DMF (2 mL) was added TEA (6.9 mg, 0.068 mmol, 2 eq.), (R)-(fluoro(2-((perfluorophenoxy)carbonyl)benzo[b]thiophen-5-yl)methyl)phosphonic acid (6, 15.6 mg, 0.034 mmol, 1 eq.). The resulting mixture was stirred for 1 h at room temperature. The crude product was purified by Prep-HPLC to afford ((R)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A21, 6 mg) as a white solid. LCMS (ESI): m/z=755 [M+H]⁺. 1H NMR (400 MHz, CD₃OD-d₄) δ (ppm) 8.08-8.01 (m, 2H), 7.88 (d, J=8.4 Hz, 1H), 7.66 (d, J=8.4 Hz, 1H), 7.37-7.26 (m, 4H), 7.24-7.17 (m, 1H), 5.65 (dd, J=45.2, 8.4 Hz, 1H), 4.79-4.68 (m, 2H), 4.66-4.58 (m, 1H), 4.52-4.40 (m, 1H), 4.07-3.99 (m, 1H), 3.96-3.89 (m, 1H), 3.86-3.73 (m, 1H), 3.61-3.49 (m, 1H), 3.00-2.86 (m, 1H), 2.81-2.68 (m, 1H), 2.65-2.06 (m, 11H), 2.03-1.60 (m, 9H), 0.59-0.44 (m, 2H). ¹⁹F NMR (376 MHz, CD₃OD-d₄) δ (ppm) −169.82 (s), −195.37-−196.18 (m). ³¹P NMR (162 MHz, CD₃OD-d₄) δ (ppm) 9.80 (d, J=76.8 Hz).

### Synthesis of (3,3-difluorocyclobutyl)methyl (((R)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C66)

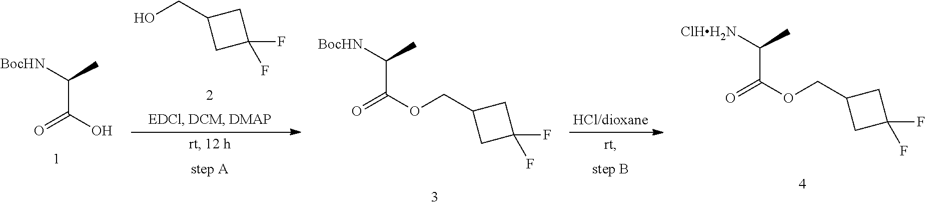

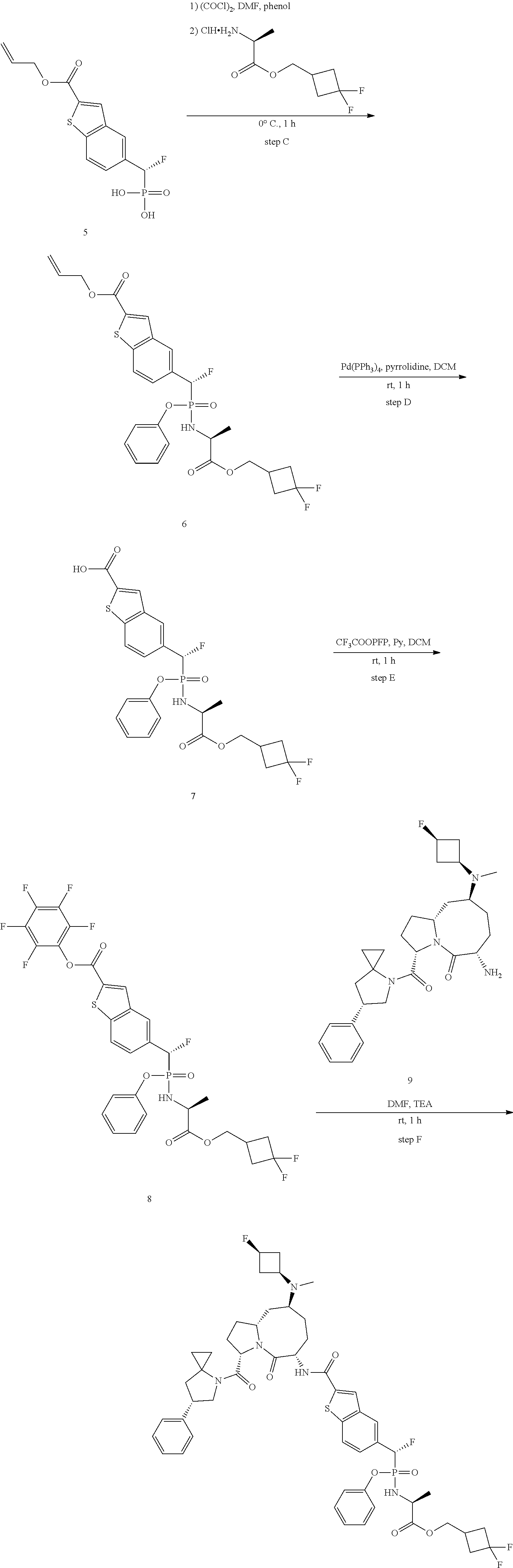

### Step A: (3,3-difluorocyclobutyl)methyl (tert-butoxycarbonyl)-L-alaninate

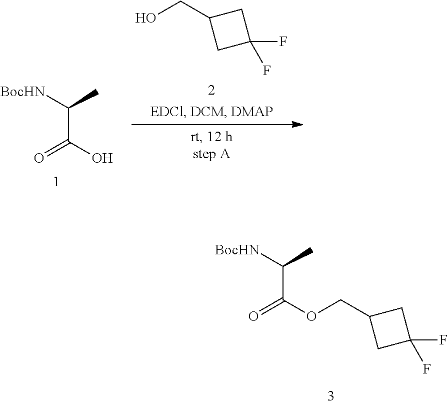 [SMILES: OCC1CC(F)(F)C1 ; C[C@H](NC(=O)OC(C)(C)C)C(=O)O ; C[C@H](NC(=O)OC(C)(C)C)C(=O)OCC1CC(F)(F)C1]

To a solution of (tert-butoxycarbonyl)-L-alanine (1, 2 g, 10.58 mmol, 1 eq.) in DCM (20 mL) was added (3,3-difluorocyclobutyl)methanol (2, 1.29 g, 10.58 mmol, 1 eq.), DMAP (1.42 g, 11.64 mmol, 1.1 eq.), and EDCI (2.23 g, 11.64 mmol, 1.1 eq.), The solution was stirred for 12 h at room temperature then H₂O (20 mL) was added to the solution, then extracted with DCM (20 mL×2), the organic layers were combined and washed with brine (20 mL), dried over Na₂SO₄, filtered and concentrated to dryness under reduced pressure. The crude product was purified by silica gel column to afford (3,3-difluorocyclobutyl)methyl (tert-butoxycarbonyl)-L-alaninate (3, 2.5 g, 8.5 mmol, 80.6% yield) as a colorless oil. LCMS (ESI): m/z=294 [M+H]⁺.

### Step B: (3,3-difluorocyclobutyl)methyl L-alaninate hydrochloride

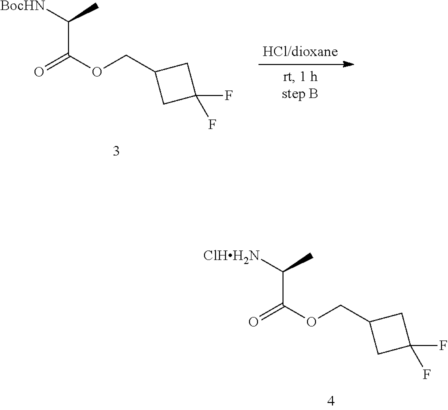

To a solution of (3,3-difluorocyclobutyl)methyl (tert-butoxycarbonyl)-L-alaninate (2, 2.5 g, 8.5 mmol) in dioxane (5 mL) was added 4M HCl in dioxane (10 mL). The resulting mixture was stirred for 1 h at room temperature then concentrated under vacuum to afford (3,3-difluorocyclobutyl)methyl L-alaninate hydrochloride (4, 1.95 g, 8.5 mmol 100% yield) as a white solid. LCMS (ESI): m/z=194 [M+H]⁺.

### Step C: allyl 5-((1R)—((((S)-1-((3,3-difluorocyclobutyl)methoxy)-1-oxopropan-2-yl)amino)(phenoxy)phosphoryl)fluoromethyl)benzo[b]thiophene-2-carboxylate

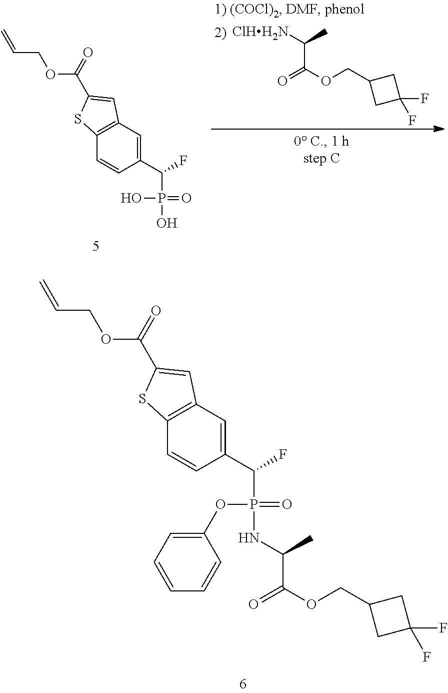

To a solution of (R)-((2-((allyloxy)carbonyl)benzo[b]thiophen-5-yl)fluoromethyl)phosphonic acid (5, 1.3 g, 3.94 mmol, 1 eq.) in DCM (20 mL) was added DMF (1 drop) and oxalyl chloride (2.5 g, 19.7 mmol, 5 eq). The resulting mixture was stirred for 1 h at room temperature, then oxalyl chloride was removed under reduced pressure. The residue was redissolved in DCM (20 mL) and cooled to 0° C. and phenol (285 mg, 3.94 mmol, 1 eq) was added to the solution followed by the dropwise addition of TEA (2 g, 19.7 mmol, 5 eq.). The mixture was stirred for 1 h at 0° C. then (3,3-difluorocyclobutyl)methyl L-alaninate hydrochloride (4, 1.80 g, 7.88 mmol, 2 eq.) was added to the solution, then stirred for 10 mins and quenched by adding H₂O (50 mL), then extracted with DCM (50 mL×2), the organic layers were combined and washed with brine (30 mL), then dried over Na₂SO₄, filtered, and concentrated to dryness under reduced pressure. The crude product was purified by silica gel chromatography column to afford allyl 5-((1R)—((((S)-1-((3,3-difluorocyclobutyl)methoxy)-1-oxopropan-2-yl)amino)(phenoxy)phosphoryl)fluoromethyl)benzo[b]thiophene-2-carboxylate (6, 800 mg, 1.38 mmol, 35% yield) as a colorless oil. LCMS (ESI): m/z=582 [M+H]⁺.

### Step D: 5-((1R)—((((S)-1-((3,3-difluorocyclobutyl)methoxy)-1-oxopropan-2-yl)amino)(phenoxy)phosphoryl)fluoromethyl)benzo[b]thiophene-2-carboxylic acid

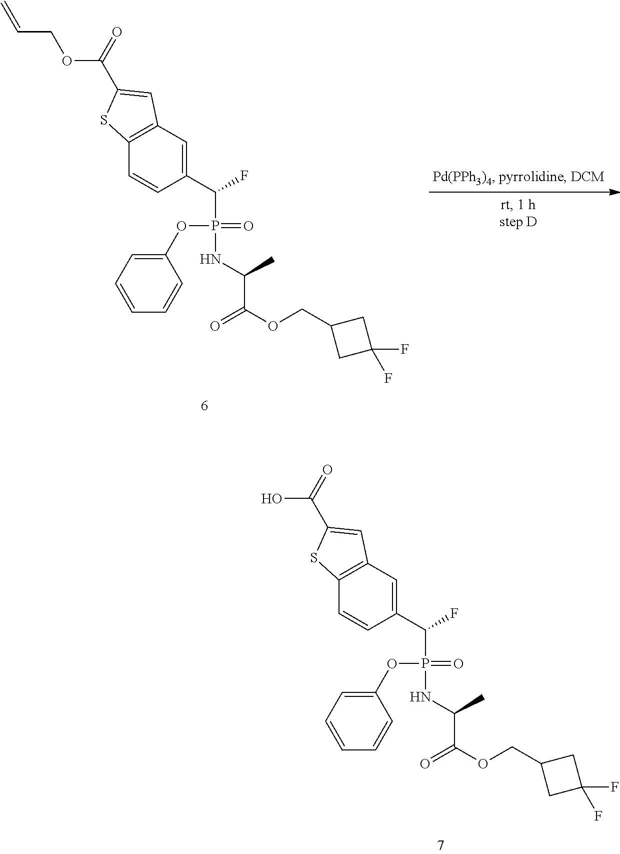 [SMILES: C=CCOC(=O)c1cc2cc([C@H](F)P(=O)(N[C@@H](C)C(=O)OCC3CC(F)(F)C3)Oc3ccccc3)ccc2s1 ; C[C@H](NP(=O)(Oc1ccccc1)[C@@H](F)c1ccc2sc(C(=O)O)cc2c1)C(=O)OCC1CC(F)(F)C1]

To a solution of allyl 5-((1R)—((((S)-1-((3,3-difluorocyclobutyl)methoxy)-1-oxopropan-2-yl)amino)(phenoxy)phosphoryl)fluoromethyl)benzo[b]thiophene-2-carboxylate (6, 800 mg, 1.38 mmol, 1 eq.) in DCM (10 mL) was added Pd(PPh₃)₄ (165 mg, 0.14 mmol, 0.1 eq.) and pyrrolidine (98 mg, 1.38 mmol, 1 eq.). The resulting mixture was stirred for 1 hr at room temperature under the atmosphere of N₂. The pH was adjusted to pH=3 with 1M HCl (aq.), then extracted with DCM (20 mL×2), the organic layers were combined and washed with brine (20 mL) dried over Na₂SO₄, filtered and concentrated to dryness under reduced pressure to afford crude product 5-((1R)—((((S)-1-((3,3-difluorocyclobutyl)methoxy)-1-oxopropan-2-yl)amino)(phenoxy)phosphoryl)fluoromethyl)benzo[b]thiophene-2-carboxylic acid (7, 700 mg, 1.29 mmol, 93% yield) as a yellow solid. LCMS (ESI): m/z=542 [M+H]⁺.

### Step E: perfluorophenyl 5-((1R)—((((S)-1-((3,3-difluorocyclobutyl)methoxy)-1-oxopropan-2-yl)amino)(phenoxy)phosphoryl)fluoromethyl)benzo[b]thiophene-2-carboxylate

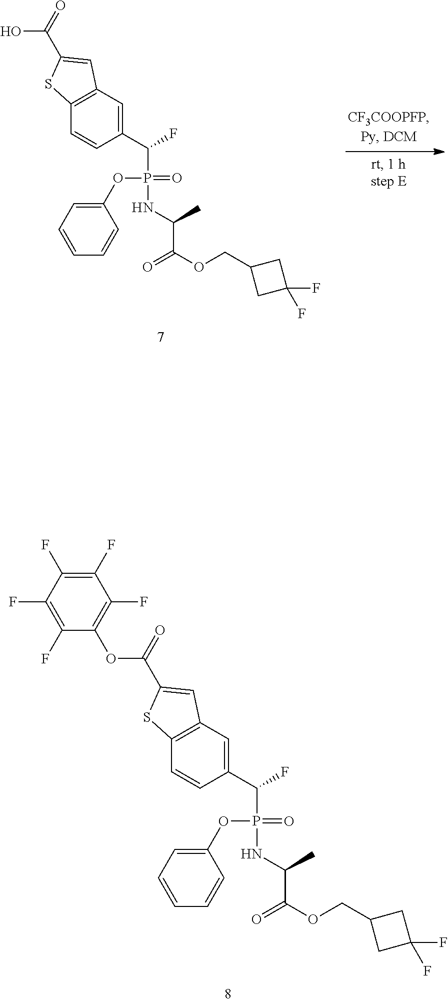 [SMILES: C[C@H](NP(=O)(Oc1ccccc1)[C@@H](F)c1ccc2sc(C(=O)O)cc2c1)C(=O)OCC1CC(F)(F)C1 ; C[C@H](NP(=O)(Oc1ccccc1)[C@@H](F)c1ccc2sc(C(=O)Oc3c(F)c(F)c(F)c(F)c3F)cc2c1)C(=O)OCC1CC(F)(F)C1]

To a solution of 5-((1R)—((((S)-1-((3,3-difluorocyclobutyl)methoxy)-1-oxopropan-2-yl)amino)(phenoxy)phosphoryl)fluoromethyl)benzo[b]thiophene-2-carboxylic acid (7, 700 mg, 1.29 mmol, 1 eq.) in DCM (10 mL) was added pyridine (306 mg, 3.87 mmol, 3 eq.) and perfluorophenyl 2,2,2-trifluoroacetate (722 mg, 2.58 mmol, 2 eq.). The resulting mixture was stirred for 1 h at room temperature, then H₂O (20 mL) was added to the solution which was then extracted with DCM (20 mL×2). The organic layers were combined and washed with brine, dried over Na₂SO₄, then concentrated under vacuum. The crude product was purified by silica gel column chromatography to afford perfluorophenyl 5-((1R)—((((S)-1-((3,3-difluorocyclobutyl)methoxy)-1-oxopropan-2-yl)amino)(phenoxy)phosphoryl)fluoromethyl)benzo[b]thiophene-2-carboxylate (8, 700 mg, 0.99 mmol, 77% yield) as a white solid. LCMS (ESI): m/z=708 [M+H]⁺.

### Step F: (3,3-difluorocyclobutyl)methyl (((R)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

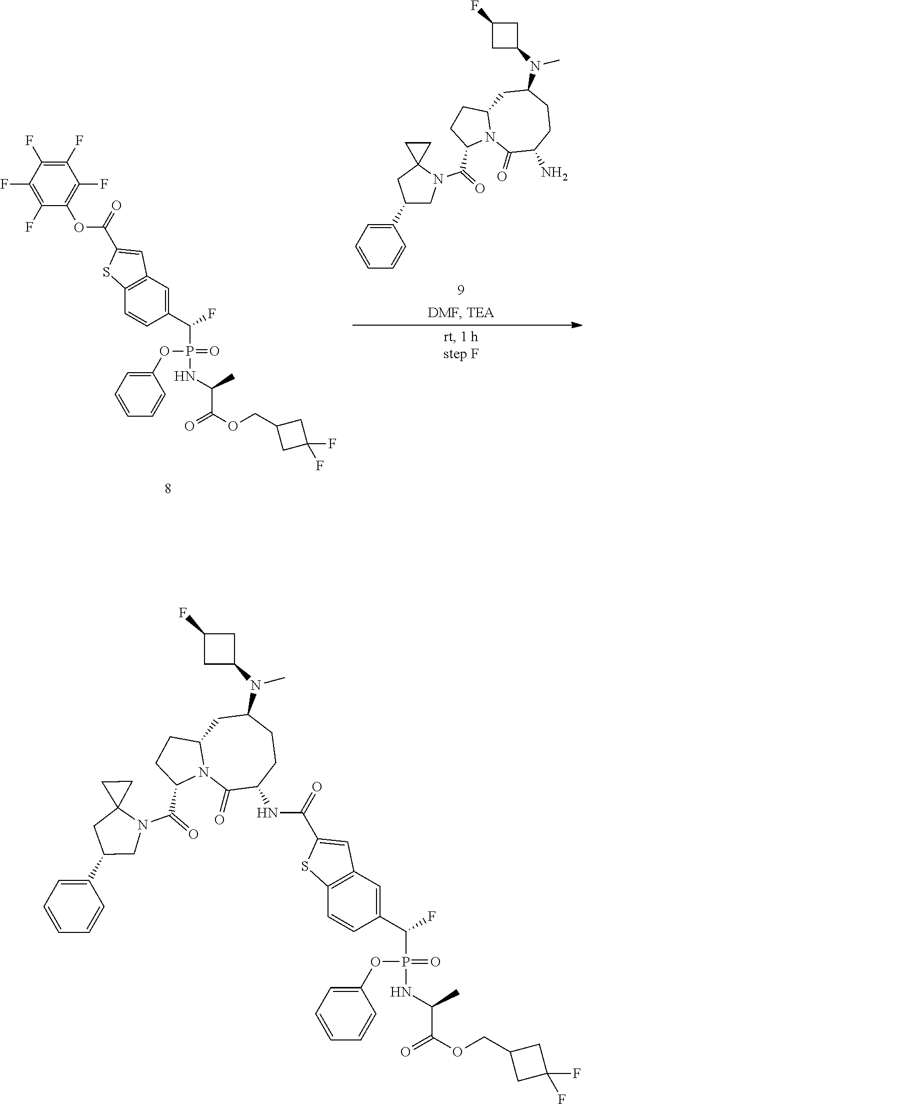 [SMILES: C[C@H](NP(=O)(Oc1ccccc1)[C@@H](F)c1ccc2sc(C(=O)Oc3c(F)c(F)c(F)c(F)c3F)cc2c1)C(=O)OCC1CC(F)(F)C1 ; CN([C@H]1CC[C@H](N)C(=O)N2[C@H](CC[C@H]2C(=O)N2C[C@H](c3ccccc3)CC23CC3)C1)[C@H]1C[C@@H](F)C1 ; C[C@H](NP(=O)(Oc1ccccc1)[C@@H](F)c1ccc2sc(C(=O)N[C@H]3CC[C@H](N(C)[C@H]4C[C@@H](F)C4)C[C@H]4CC[C@@H](C(=O)N5C[C@H](c6ccccc6)CC56CC6)N4C3=O)cc2c1)C(=O)OCC1CC(F)(F)C1]

To a solution of perfluorophenyl 5-((1R)—((((S)-1-((3,3-difluorocyclobutyl)methoxy)-1-oxopropan-2-yl)amino)(phenoxy)phosphoryl)fluoromethyl)benzo[b]thiophene-2-carboxylate (8, 20 mg, 0.028 mml, 1 eq.) in DMF (3 mL) was added (3S,6S,9S,10aR)-6-amino-9-(((1s,3R)-3-fluorocyclobutyl)(methyl)amino)-3-((S)-6-phenyl-4-azaspiro[2.4]heptane-4-carbonyl)octahydropyrrolo[1,2-a]azocin-5(1H)-one (9, 14 mg, 0.029 mmol, 1 eq.), TEA (5.7 mg, 0.056 mmol, 2 eq). The resulting mixture was stirred for 1 h at room temperature. The crude product was purified by Prep-HPLC to afford (3,3-difluorocyclobutyl)methyl (((R)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C66, 18 mg, 0.018 mmol, 64% yield) as a white solid. LCMS (ESI): m/z=1006 [M+H]⁺. ¹H NMR (400 MHz, DMSO-d6) δ (ppm) 8.89-8.71 (m, 1H), 8.27 (s, 1H), 8.12-7.99 (m, 2H), 7.65-7.56 (m, 1H), 7.39-7.27 (m, 6H), 7.24-7.13 (m, 4H), 6.31-6.21 (m, 1H), 6.18-6.10 (m, 1H), 4.90-4.66 (m, 2H), 4.52-4.43 (m, 1H), 4.33-4.24 (m, 1H), 4.15-3.70 (m, 5H), 3.59-3.48 (m, 1H), 3.06-2.93 (m, 1H), 2.63-2.53 (m, 3H), 2.49-1.45 (m, 24H), 1.15 (d, J=7.0 Hz, 3H), 0.60-0.36 (m, 2H). ¹⁹F NMR (377 MHz, DMSO-d6) δ (ppm) −80.84-−82.82 (m), −91.58-−93.03 (m), −165.27 (s), −194.62 (d, J=82.8 Hz), −196.26 (d, J=86.1 Hz). ³¹P NMR (162 MHz, DMSO-d6) δ (ppm) 17.25 (d, J=83.1 Hz), 17.12 (d, J=86.1 Hz).

### Synthesis of ((S)-(2-(((3S,6S,9S,10aR)-9-(ethyl((1s,3R)-3-fluorocyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A34) and propyl (((S)-(2-(((3S,6S,9S,10aR)-9-(ethyl((1s,3R)-3-fluorocyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C56)

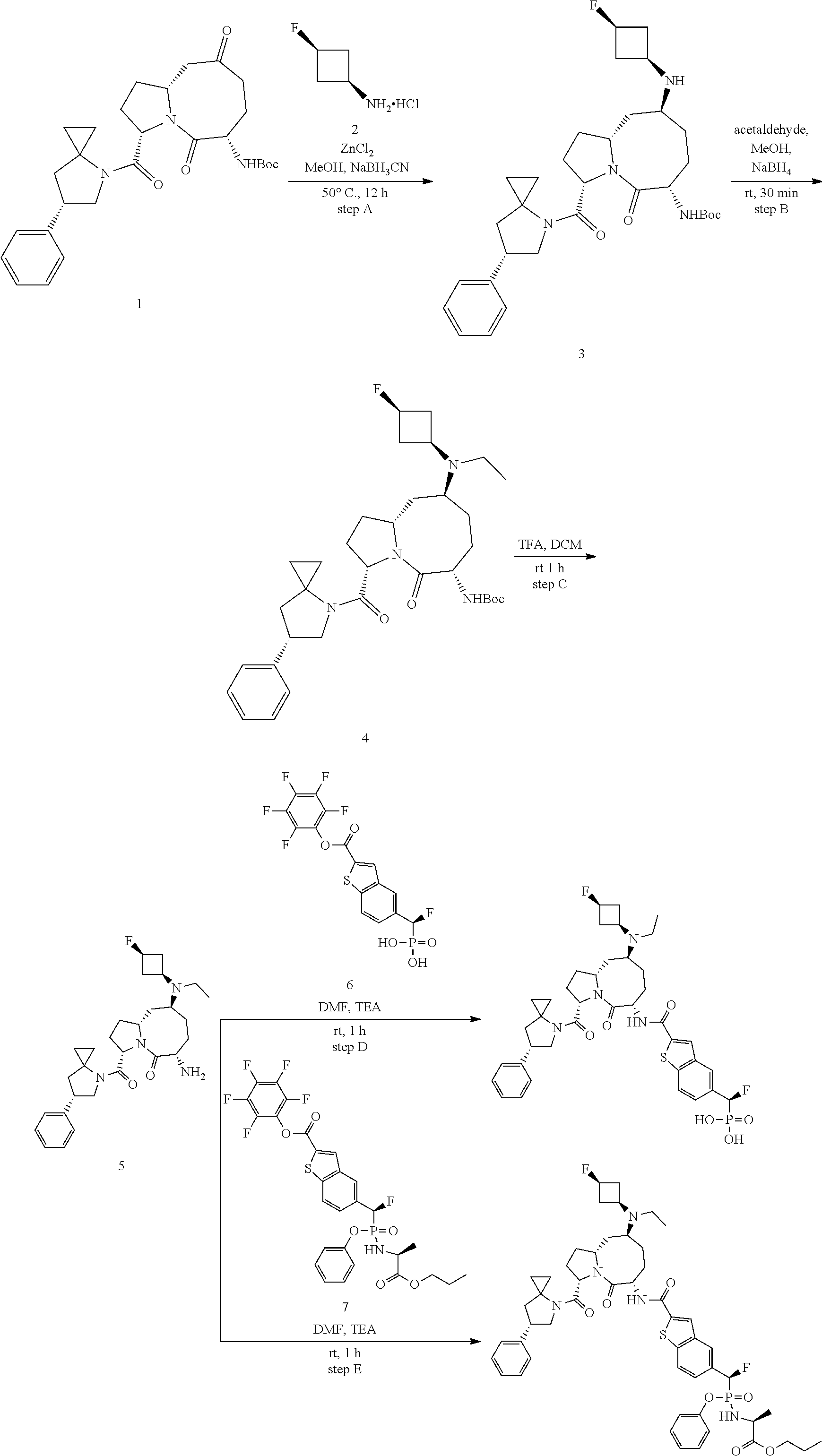

### Step A: tert-butyl ((3S,6S,9S,10aR)-9-(((1s,3R)-3-fluorocyclobutyl)amino)-5-oxo-3-((S)-6-phenyl-4-azaspiro[2.4]heptane-4-carbonyl)decahydropyrrolo[1,2-a]azocin-6-yl)carbamate

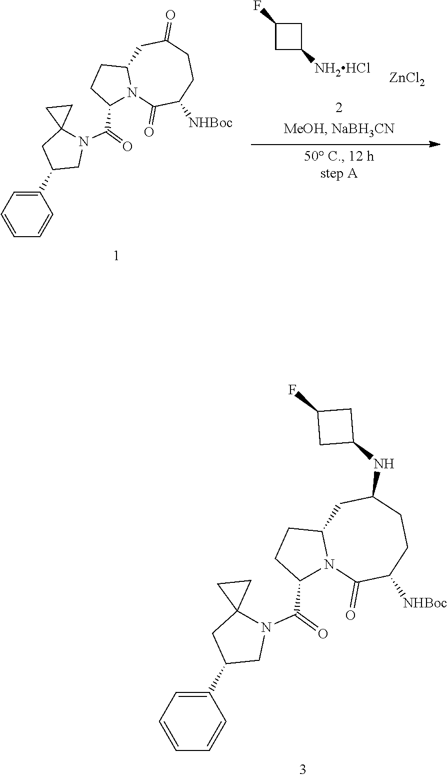

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (1, 3 g, 6.06 mmol, 1 eq.) in MeOH (60 mL) was added (1s,3s)-3-fluorocyclobutan-1-amine hydrochloride (2, 1.14 g, 9.09 mmol, 1.5 eq.), ZnCl₂ (8.3 g, 60.6 mmol, 10 eq.) and the resulting mixture was stirred for 2 hr at 50° C. under the atmosphere of N₂. Then NaBH₃CN (3.81 g, 60.6 mmol, 10 eq.) was added portion wise every 30 mins (2 eq. (762 mg each time) added 5 times). The solution was stirred for 12 hr at 50° C. then cooled to room temperature. The solution was used to next step without further workup. LCMS (ESI): m/z=569 [M+H]⁺.

### Step B: tert-butyl ((3S,6S,9S,10aR)-9-(ethyl((1s,3R)-3-fluorocyclobutyl)amino)-5-oxo-3-((S)-6-phenyl-4-azaspiro[2.4]heptane-4-carbonyl)decahydropyrrolo[1,2-a]azocin-6-yl)carbamate

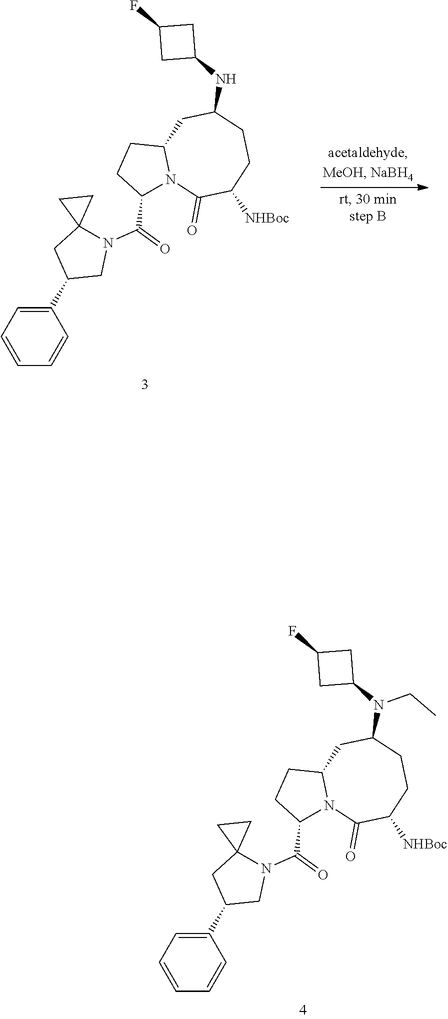 [SMILES: CC(C)(C)OC(=O)N[C@H]1CC[C@H](N[C@H]2C[C@@H](F)C2)C[C@H]2CC[C@@H](C(=O)N3C[C@H](c4ccccc4)CC34CC4)N2C1=O ; CCN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](F)C1]

To the above mixture (step A) was added acetaldehyde (800 mg, 18.18 mmol, 3 eq.) at room temperature, the mixture was stirred for 30 min, then NaBH₄ (1.15 g, 30.3 mmol, 5 eq.) was added in portions, the mixture was stirred for another 30 min, LCMS show that the reaction was completed. The reaction was quenched by NH₄Cl (sat. aq), diluted with H₂O (200 mL) and extracted with EtOAc (100 mL×3), the organic solution was dried over Na₂SO₄, filtered, then concentrated under vacuum. The crude product was purified by C18 column (MeCN:H₂O (0.1% ammonium hydroxide)=5%-70%) to afford tert-butyl ((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamate (4, 3 g, 5.03 mmol, 83% yield two steps) as a white solid. LCMS (ESI): m/z=597 [M+H]⁺.

### Step C: (3S,6S,9S,10aR)-6-amino-9-(ethyl((1s,3R)-3-fluorocyclobutyl)amino)-3-((S)-6-phenyl-4-azaspiro[2.4]heptane-4-carbonyl)octahydropyrrolo[1,2-a]azocin-5(1H)-one

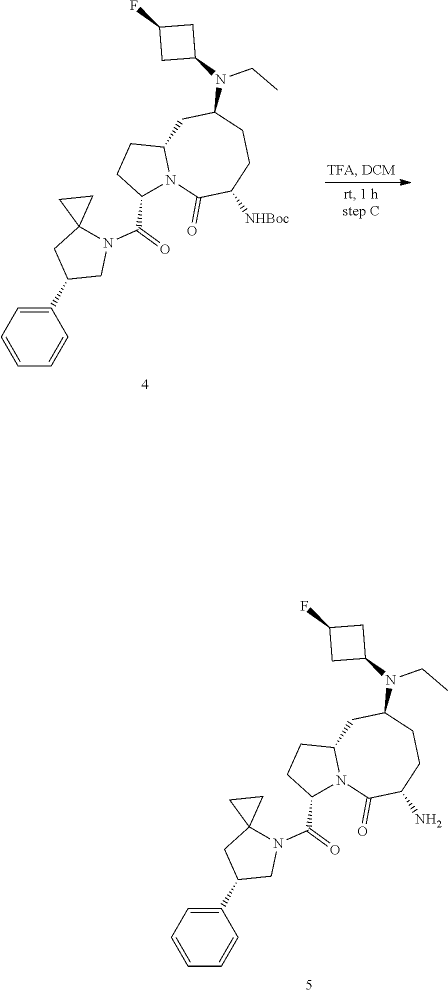 [SMILES: CCN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](F)C1 ; CCN([C@H]1CC[C@H](N)C(=O)N2[C@H](CC[C@H]2C(=O)N2C[C@H](c3ccccc3)CC23CC3)C1)[C@H]1C[C@@H](F)C1]

To a solution of tert-butyl ((3S,6S,9S,10aR)-9-(ethyl((1s,3R)-3-fluorocyclobutyl)amino)-5-oxo-3-((S)-6-phenyl-4-azaspiro[2.4]heptane-4-carbonyl)decahydropyrrolo[1,2-a]azocin-6-yl)carbamate (4, 20 mg, 0.034 mmol) in DCM (2 mL) was added TFA (2 mL). The resulting mixture was stirred for 1 h at room temperature then concentrated to dryness under reduced pressure to afford crude product (3S,6S,9S,10aR)-6-amino-9-(ethyl((1s,3R)-3-fluorocyclobutyl)amino)-3-((S)-6-phenyl-4-azaspiro[2.4]heptane-4-carbonyl)octahydropyrrolo[1,2-a]azocin-5(1H)-one (5, 20 mg, TFA salt) as a yellow oil which was used to the next step without further purification. LCMS (ESI): m/z=497 [M+H]⁺.

### Step D: ((S)-(2-(((3S,6S,9S,10aR)-9-(ethyl((1s,3R)-3-fluorocyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid

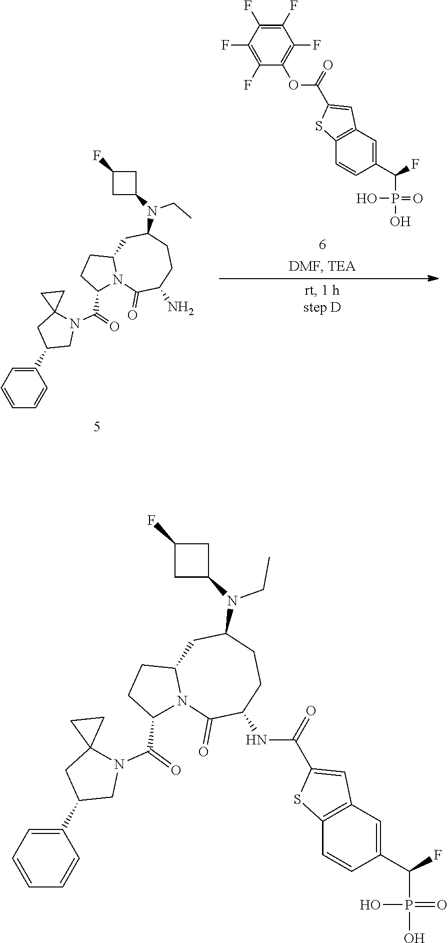 [SMILES: CCN([C@H]1CC[C@H](N)C(=O)N2[C@H](CC[C@H]2C(=O)N2C[C@H](c3ccccc3)CC23CC3)C1)[C@H]1C[C@@H](F)C1 ; O=C(Oc1c(F)c(F)c(F)c(F)c1F)c1cc2cc([C@@H](F)P(=O)(O)O)ccc2s1 ; CCN([C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1)[C@H]1C[C@@H](F)C1]

To a solution of (3S,6S,9S,10aR)-6-amino-9-(ethyl((1s,3R)-3-fluorocyclobutyl)amino)-3-((S)-6-phenyl-4-azaspiro[2.4]heptane-4-carbonyl)octahydropyrrolo[1,2-a]azocin-5(1H)-one (5, 20 mg, TFA salt) in DMF (2 mL) was added TEA (6.8 mg, 0.067 mmol, 2 eq.), (S)-(fluoro(2-((perfluorophenoxy)carbonyl)benzo[b]thiophen-5-yl)methyl)phosphonic acid (6, 15.3 mg, 0.034 mmol, 1 eq.). The resulting mixture was stirred for 1 h at room temperature then purified by Prep-HPLC to afford ((S)-(2-(((3S,6S,9S,10aR)-9-(ethyl((1s,3R)-3-fluorocyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A34, 13 mg) as a white solid. LCMS (ESI): m/z=769 [M+H]⁺. ¹H NMR (400 MHz, CD₃OD-d₄) δ (ppm) 8.09-8.02 (m, 2H), 7.87 (d, J=8.4 Hz, 1H), 7.62 (d, J=8.5 Hz, 1H), 7.38-7.25 (m, 4H), 7.23-7.16 (m, 1H), 5.76-5.52 (m, 1H), 4.77-4.37 (m, 4H), 4.09-3.85 (m, 2H), 3.79-3.64 (m, 1H), 3.62-3.49 (m, 1H), 3.08-1.54 (m, 21H), 1.33-1.20 (m, 3H), 0.52 (s, 2H). ³¹P NMR (162 MHz, CD₃OD-d₄) δ (ppm) 9.79 (d, J=75.4 Hz). ¹⁹F NMR (376 MHz, CD₃OD-d₄) δ (ppm) −169.78-−170.16 (m), −196.02 (d, J=75.9 Hz).

### Step E: propyl (((S)-(2-(((3S,6S,9S,10aR)-9-(ethyl((1s,3R)-3-fluorocyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate

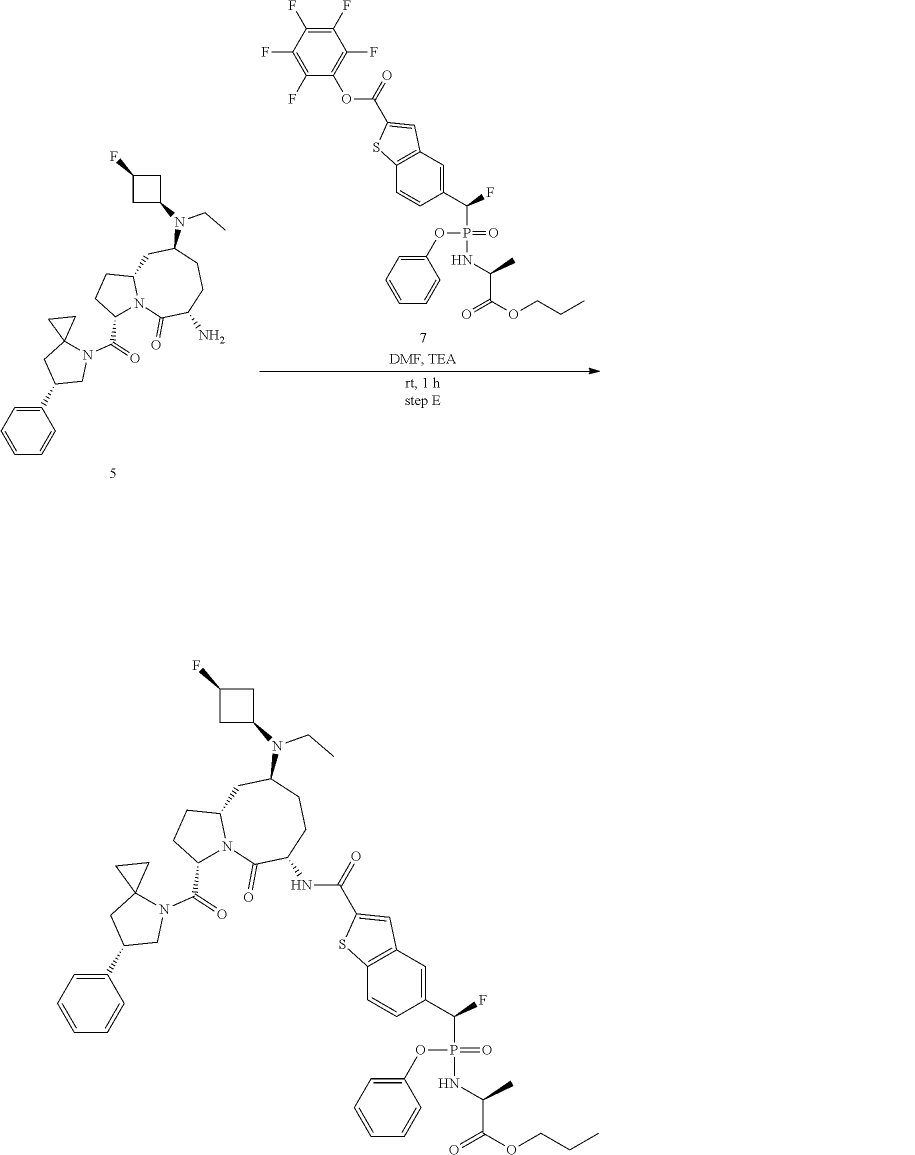 [SMILES: CCN([C@H]1CC[C@H](N)C(=O)N2[C@H](CC[C@H]2C(=O)N2C[C@H](c3ccccc3)CC23CC3)C1)[C@H]1C[C@@H](F)C1 ; CCCOC(=O)[C@H](C)NP(=O)(Oc1ccccc1)[C@H](F)c1ccc2sc(C(=O)Oc3c(F)c(F)c(F)c(F)c3F)cc2c1 ; CCCOC(=O)[C@H](C)NP(=O)(Oc1ccccc1)[C@H](F)c1ccc2sc(C(=O)N[C@H]3CC[C@H](N(CC)[C@H]4C[C@@H](F)C4)C[C@H]4CC[C@@H](C(=O)N5C[C@H](c6ccccc6)CC56CC6)N4C3=O)cc2c1]

To a solution of (3S,6S,9S,10aR)-6-amino-9-(ethyl((1s,3R)-3-fluorocyclobutyl)amino)-3-((S)-6-phenyl-4-azaspiro[2.4]heptane-4-carbonyl)octahydropyrrolo[1,2-a]azocin-5(1H)-one (5, 20 mg, TFA salt) in DMF (2 mL) was added TEA (6.8 mg, 0.067 mmol, 2 eq.), perfluorophenyl 5-((1S)-fluoro((((S)-1-oxo-1-propoxypropan-2-yl)amino)(phenoxy)phosphoryl)methyl)benzo[b]thiophene-2-carboxylate (7, 21.6 mg, 0.034 mmol, 1 eq.). The resulting mixture was stirred for 1 h at room temperature then crude product was purified by Prep-HPLC to afford propyl (((S)-(2-(((3S,6S,9S,10aR)-9-(ethyl((1s,3R)-3-fluorocyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate. (Compound C56, 15 mg) as a white solid. LCMS (ESI): m/z=958 [M+H]⁺. ¹H NMR (400 MHz, DMSO-d₆) δ (ppm) 8.84-8.73 (m, 1H), 8.31-8.23 (m, 1H), 8.11-8.01 (m, 2H), 7.64-7.56 (m, 1H), 7.41-7.28 (m, 6H), 7.23-7.11 (m, 4H), 6.29-6.08 (m, 2H), 4.88-4.66 (m, 2H), 4.47 (t, J=8.5 Hz, 1H), 4.33-4.23 (m, 1H), 4.17-4.09 (m, 1H), 3.94-3.69 (m, 4H), 3.60-3.48 (m, 1H), 3.09-2.98 (m, 1H), 2.74-2.66 (m, 1H), 2.61-2.53 (m, 1H), 2.45-2.38 (m, 1H), 2.37-1.89 (m, 10H), 1.88-1.37 (m, 11H), 1.18-1.16 (m, 1H), 1.00-0.93 (m, 4H), 0.83-0.78 (m, 3H), 0.55-0.41 (m, 2H). ¹⁹F NMR (377 MHz, DMSO-d₆) −165.89 (s), −194.18 (d, J=82.6 Hz), −196.07 (d, J=86.6 Hz). ³¹P NMR (162 MHz, DMSO-d6) δ (ppm) 17.32 (d, J=86.6 Hz), 16.94 (d, J=82.5 Hz).

### Synthesis of ((R)-fluoro(2-(((3S,6S,9S,10aR)-9-(methyl((3-methyloxetan-3-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A56) and cyclobutyl ((R)-((R)-fluoro(2-(((3S,6S,9S,10aR)-9-(methyl((3-methyloxetan-3-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C172)

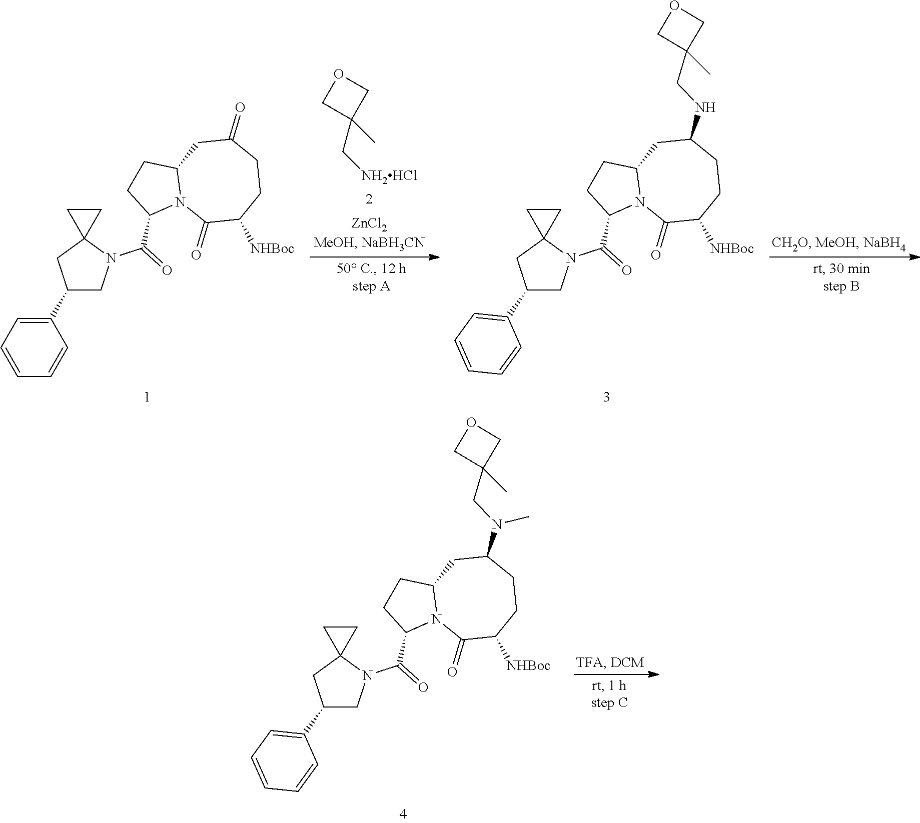

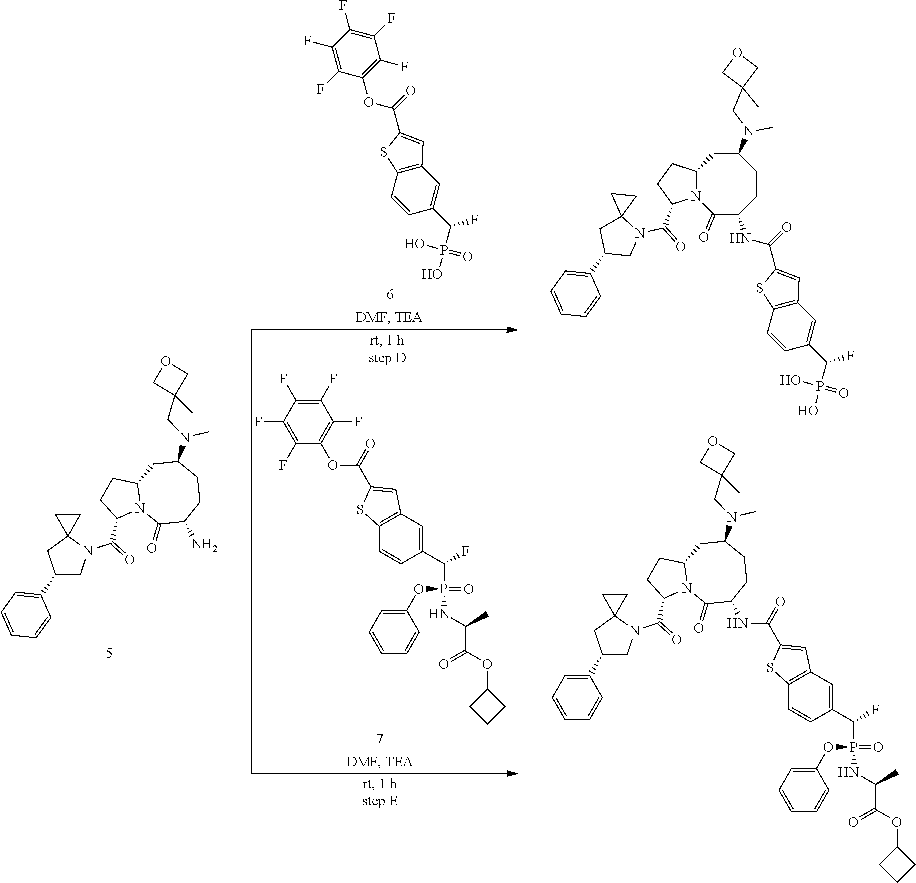

### Step A: tert-butyl ((3S,6S,9S,10aR)-9-(((3-methyloxetan-3-yl)methyl)amino)-5-oxo-3-((S)-6-phenyl-4-azaspiro[2.4]heptane-4-carbonyl)decahydropyrrolo[1,2-a]azocin-6-yl)carbamate

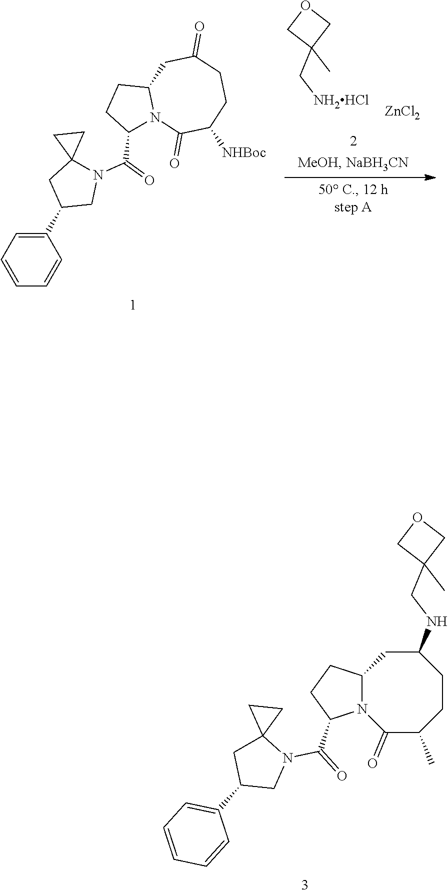

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (1, 1 g, 2.02 mmol, 1 eq.) in MeOH (20 mL) was added (3-methyloxetan-3-yl)methanamine hydrochloride (2, 417 mg, 3.03 mmol, 1.5 eq.), ZnCl₂ (2.77 g, 20.2 mmol, 10 eq.), the resulting mixture was stirred for 2 hr. at 50° C. under the atmosphere of N₂. Then NaBH₃CN (1.27 g, 20.2 mmol, 10 eq.) was added portion wise every 30 mins (2 eq. (254 mg each time) added 5 times). The mixture was stirred for 12 hr. at 50° C., LCMS show that the reaction was completed, then cooled to room temperature, The solution was used to next step without further workup. LCMS (ESI): m/z=581 [M+H]⁺.

### Step B: tert-butyl ((3S,6S,9S,10aR)-9-(methyl((3-methyloxetan-3-yl)methyl)amino)-5-oxo-3-((S)-6-phenyl-4-azaspiro[2.4]heptane-4-carbonyl)decahydropyrrolo[1,2-a]azocin-6-yl)carbamate

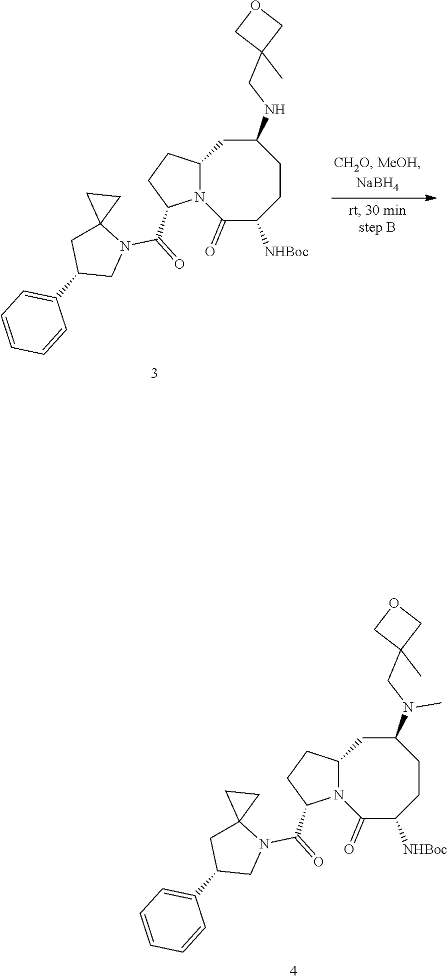 [SMILES: CC1(CN[C@H]2CC[C@H](NC(=O)OC(C)(C)C)C(=O)N3[C@H](CC[C@H]3C(=O)N3C[C@H](c4ccccc4)CC34CC4)C2)COC1 ; CN(CC1(C)COC1)[C@H]1CC[C@H](NC(=O)OC(C)(C)C)C(=O)N2[C@H](CC[C@H]2C(=O)N2C[C@H](c3ccccc3)CC23CC3)C1]

To the above mixture (step A) was added 40% Formaldehyde (3 mL) at room temperature, the mixture was stirred for 30 min, then NaBH₄ (383 mg, 10.1 mmol, 5 eq.) was added in portions, the mixture was stirred for another 30 min, LCMS show that the reaction was completed. The reaction was quenched by NH₄Cl (sat. aq), diluted with H₂O (100 mL) and extracted with EtOAc (50 mL×3), the organic solution was dried over Na₂SO₄, then concentrated under vacuum, the crude product was purified by C18 column (MeCN:H₂O (0.1% ammonium hydroxide)=5%-70%) to afford tert-butyl ((3S,6S,9S,10aR)-9-(methyl((3-methyloxetan-3-yl)methyl)amino)-5-oxo-3-((S)-6-phenyl-4-azaspiro[2.4]heptane-4-carbonyl)decahydropyrrolo[1,2-a]azocin-6-yl)carbamate (4, 3 g, 5.04 mmol, 83% yield two steps) as a white solid. LCMS (ESI): m/z=595 [M+H]⁺.

### Step C: (3S,6S,9S,10aR)-6-amino-9-(methyl((3-methyloxetan-3-yl)methyl)amino)-3-((S)-6-phenyl-4-azaspiro[2.4]heptane-4-carbonyl)octahydropyrrolo[1,2-a]azocin-5(1H)-one

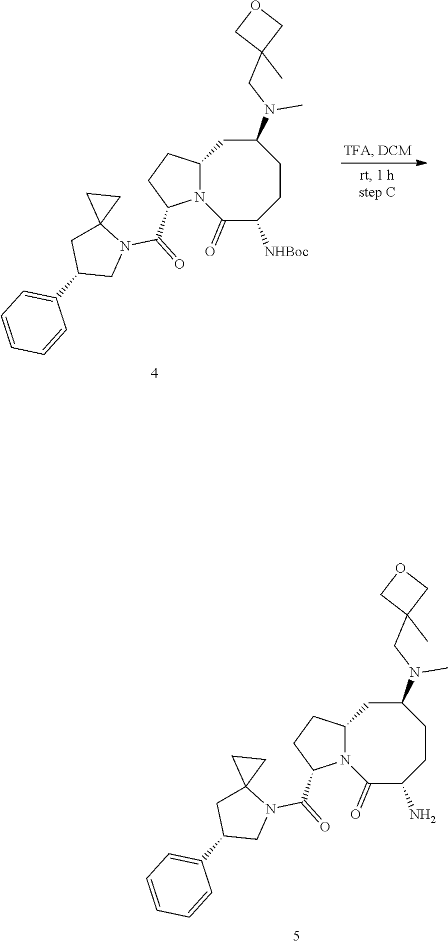 [SMILES: CN(CC1(C)COC1)[C@H]1CC[C@H](NC(=O)OC(C)(C)C)C(=O)N2[C@H](CC[C@H]2C(=O)N2C[C@H](c3ccccc3)CC23CC3)C1 ; CN(CC1(C)COC1)[C@H]1CC[C@H](N)C(=O)N2[C@H](CC[C@H]2C(=O)N2C[C@H](c3ccccc3)CC23CC3)C1]

To a solution of tert-butyl ((3S,6S,9S,10aR)-9-(methyl((3-methyloxetan-3-yl)methyl)amino)-5-oxo-3-((S)-6-phenyl-4-azaspiro[2.4]heptane-4-carbonyl)decahydropyrrolo[1,2-a]azocin-6-yl)carbamate (4, 20 mg, 0.034 mmol) in DCM (2 mL) was added TFA (2 mL). The resulting mixture was stirred for 1 h at room temperature, then concentrated to dryness under reduced pressure to afford crude product (3S,6S,9S,10aR)-6-amino-9-(methyl((3-methyloxetan-3-yl)methyl)amino)-3-((S)-6-phenyl-4-azaspiro[2.4]heptane-4-carbonyl)octahydropyrrolo[1,2-a]azocin-5(1H)-one (5, 20 mg, TFA salt) as a yellow oil which was used to the next step without further purification. LCMS (ESI): m/z=495 [M+H]⁺.

### Step D: ((R)-fluoro(2-(((3S,6S,9S,10aR)-9-(methyl((3-methyloxetan-3-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

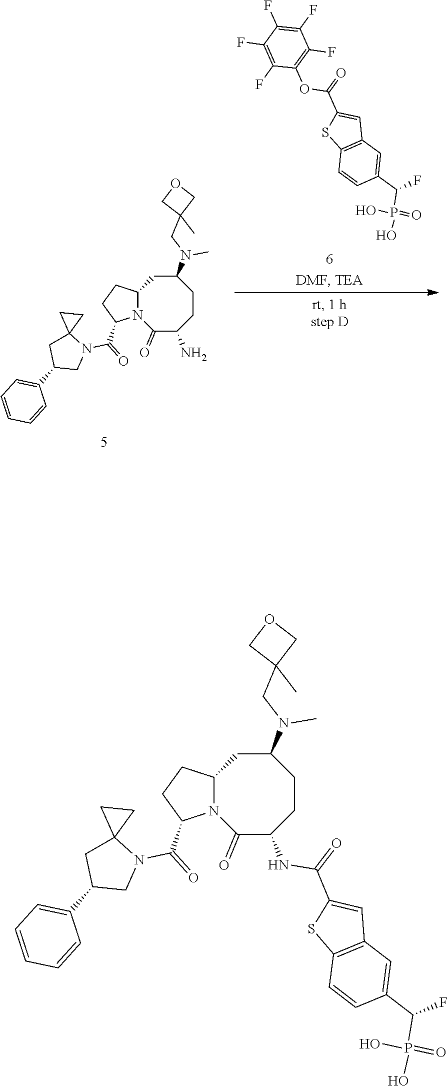 [SMILES: CN(CC1(C)COC1)[C@H]1CC[C@H](N)C(=O)N2[C@H](CC[C@H]2C(=O)N2C[C@H](c3ccccc3)CC23CC3)C1 ; O=C(Oc1c(F)c(F)c(F)c(F)c1F)c1cc2cc([C@H](F)P(=O)(O)O)ccc2s1 ; CN(CC1(C)COC1)[C@H]1CC[C@H](NC(=O)c2cc3cc([C@H](F)P(=O)(O)O)ccc3s2)C(=O)N2[C@H](CC[C@H]2C(=O)N2C[C@H](c3ccccc3)CC23CC3)C1]

To a solution of (3S,6S,9S,10aR)-6-amino-9-(methyl((3-methyloxetan-3-yl)methyl)amino)-3-((S)-6-phenyl-4-azaspiro[2.4]heptane-4-carbonyl)octahydropyrrolo[1,2-a]azocin-5(1H)-one (5, 20 mg, TFA salt) in DMF (2 mL) was added TEA (6.8 mg, 0.067 mmol, 2 eq.), (R)-(fluoro(2-((perfluorophenoxy)carbonyl)benzo[b]thiophen-5-yl)methyl)phosphonic acid (6, 15.3 mg, 0.034 mmol, 1 eq.). The resulting mixture was stirred for 1 h at room temperature then was purified by Prep-HPLC to afford ((R)-fluoro(2-(((3S,6S,9S,10aR)-9-(methyl((3-methyloxetan-3-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A56, 20 mg) as a white solid. LCMS (ESI): m/z=767 [M+H]⁺. ¹H NMR (400 MHz, CD₃OD-d₄) δ (ppm) 8.09-7.99 (m, 2H), 7.88 (d, J=8.5 Hz, 1H), 7.65 (d, J=8.5 Hz, 1H), 7.36-7.26 (m, 4H), 7.23-7.18 (m, 1H), 5.64 (dd, J=45.3, 8.3 Hz, 1H), 4.75-4.67 (m, 1H), 4.63-4.54 (m, 3H), 4.44-4.30 (m, 3H), 4.06-3.87 (m, 2H), 3.61-3.46 (m, 2H), 3.26-3.07 (m, 2H), 2.55 (s, 3H), 2.48-2.39 (m, 1H), 2.30 (t, J=11.4 Hz, 1H), 2.22-1.92 (m, 7H), 1.92-1.75 (m, 4H), 1.66-1.59 (m, 1H), 1.52 (s, 3H), 0.57-0.44 (m, 2H). ¹⁹F NMR (377 MHz, CD₃OD-d₄) δ (ppm) −195.52-−195.88 (m). ³¹P NMR (162 MHz, CD₃OD-d₄) δ (ppm) 9.71 (d, J=76.3 Hz).

### Step E: cyclobutyl ((R)-((R)-fluoro(2-(((3S,6S,9S,10aR)-9-(methyl((3-methyloxetan-3-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

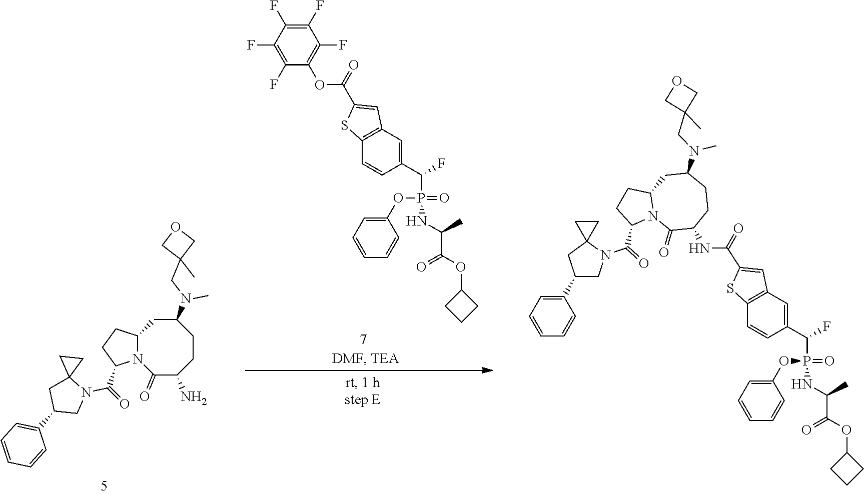 [SMILES: CN(CC1(C)COC1)[C@H]1CC[C@H](N)C(=O)N2[C@H](CC[C@H]2C(=O)N2C[C@H](c3ccccc3)CC23CC3)C1 ; C[C@H](N[P@](=O)(Oc1ccccc1)[C@@H](F)c1ccc2sc(C(=O)Oc3c(F)c(F)c(F)c(F)c3F)cc2c1)C(=O)OC1CCC1 ; C[C@H](N[P@](=O)(Oc1ccccc1)[C@@H](F)c1ccc2sc(C(=O)N[C@H]3CC[C@H](N(C)CC4(C)COC4)C[C@H]4CC[C@@H](C(=O)N5C[C@H](c6ccccc6)CC56CC6)N4C3=O)cc2c1)C(=O)OC1CCC1]

To a solution of (3S,6S,9S,10aR)-6-amino-9-(methyl((3-methyloxetan-3-yl)methyl)amino)-3-((S)-6-phenyl-4-azaspiro[2.4]heptane-4-carbonyl)octahydropyrrolo[1,2-a]azocin-5(1H)-one (5, 30 mg, TFA salt) in DMF (2 mL) was added TEA (10.2 mg, 0.1 mmol, 2 eq.), perfluorophenyl 5-((R)-((R)—(((S)-1-cyclobutoxy-1-oxopropan-2-yl)amino)(phenoxy)phosphoryl)fluoromethyl)benzo[b]thiophene-2-carboxylate (7, 33.15 mg, 0.051 mmol, 1 eq.). The resulting mixture was stirred for 1 h at room temperature then purified by Prep-HPLC to afford cyclobutyl ((R)-((R)-fluoro(2-(((3S,6S,9S,10aR)-9-(methyl((3-methyloxetan-3-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C172, 15 mg) as a white solid. LCMS (ESI): m/z=968 [M+H]⁺. ¹H NMR (400 MHz, DMSO-d₆) δ (ppm) 8.77 (d, J=7.7 Hz, 1H), 8.27 (s, 1H), 8.10-7.99 (m, 2H), 7.61 (d, J=8.6 Hz, 1H), 7.40-7.27 (m, 6H), 7.23-7.12 (m, 4H), 6.27-6.04 (m, 2H), 4.81-4.65 (m, 2H), 4.52-4.43 (m, 1H), 4.40 (d, J=5.4 Hz, 1H), 4.36 (d, J=5.4 Hz, 1H), 4.32-4.24 (m, 1H), 4.19 (d, J=5.4 Hz, 1H), 4.14-4.05 (m, 2H), 3.84-3.67 (m, 2H), 3.59-3.48 (m, 1H), 2.88-2.78 (m, 1H), 2.58-2.52 (m, 1H), 2.39-2.31 (m, 1H), 2.22-1.50 (m, 23H), 1.28 (s, 3H), 1.11 (d, J=7.0 Hz, 3H), 0.54-0.38 (m, 2H). ¹⁹F NMR (376 MHz, DMSO-d₆) δ (ppm) −194.33 (d, J=82.8 Hz). ³¹P NMR (162 MHz, DMSO-d₆) δ (ppm) 17.28 (d, J=82.9 Hz).

### Synthesis of ((S)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A65) and propyl ((S)-((S)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C210)

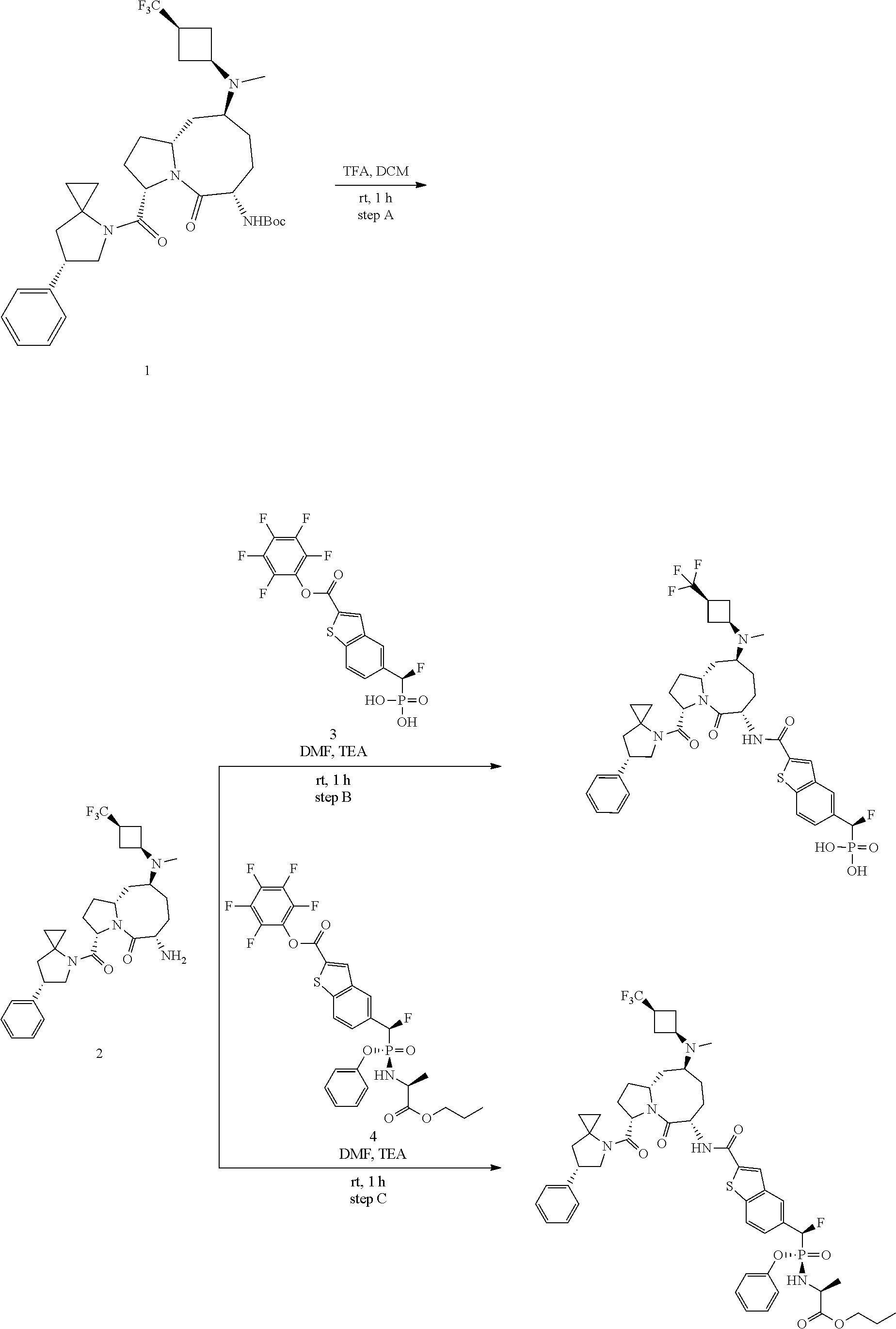

### Step A: (3S,6S,9S,10aR)-6-amino-9-(methyl((1s,3R)-3-(trifluoromethyl)cyclobutyl)amino)-3-((S)-6-phenyl-4-azaspiro[2.4]heptane-4-carbonyl)octahydropyrrolo[1,2-a]azocin-5(1H)-one

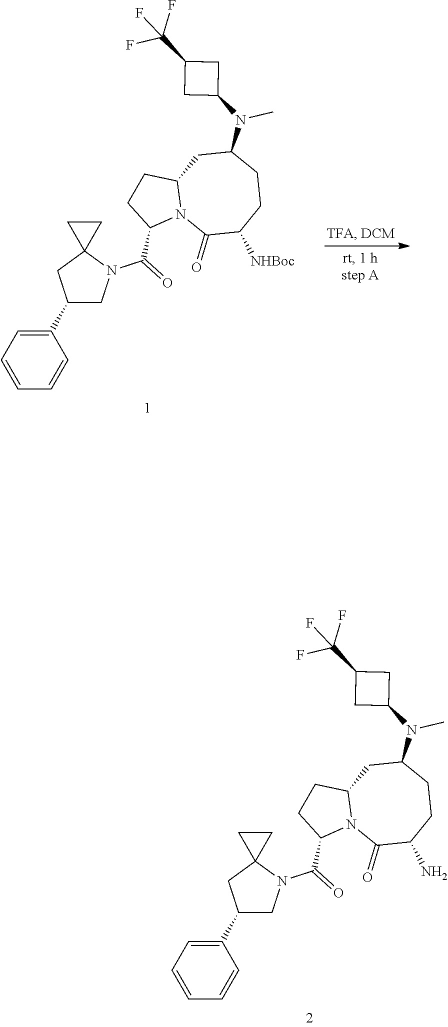 [SMILES: CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](C(F)(F)F)C1 ; CN([C@H]1CC[C@H](N)C(=O)N2[C@H](CC[C@H]2C(=O)N2C[C@H](c3ccccc3)CC23CC3)C1)[C@H]1C[C@@H](C(F)(F)F)C1]

To a solution of tert-butyl ((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamate (1, 30 mg, 0.047 mmol) in DCM (2 mL) was added TFA (2 mL). The resulting mixture was stirred for 1 h at room temperature and the resulting mixture was concentrated to dryness under reduced pressure to afford crude product (3S,6S,9S,10aR)-6-amino-9-(methyl((1s,3R)-3-(trifluoromethyl)cyclobutyl)amino)-3-((S)-6-phenyl-4-azaspiro[2.4]heptane-4-carbonyl)octahydropyrrolo[1,2-a]azocin-5(1H)-one (2, 30 mg, TFA salt) as a yellow oil which was used to the next step without further purification. LCMS (ESI): m/z=533 [M+H]⁺.

### Step B: ((S)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

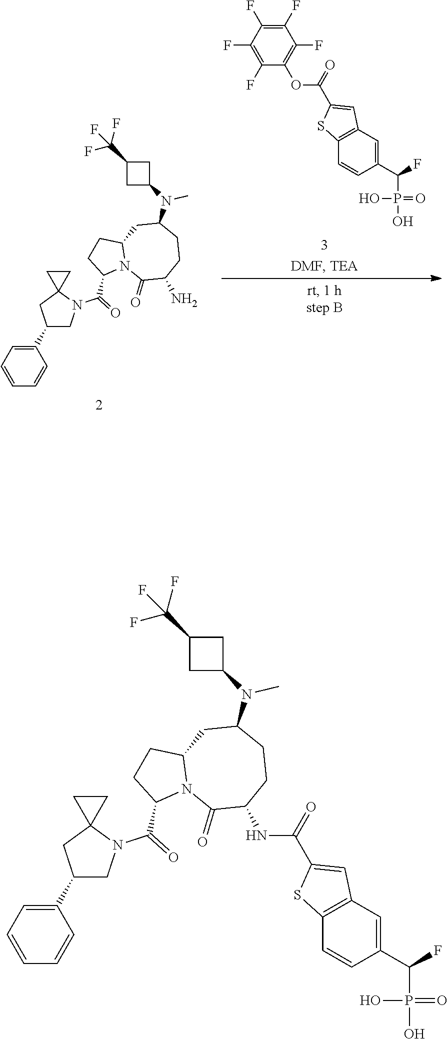 [SMILES: CN([C@H]1CC[C@H](N)C(=O)N2[C@H](CC[C@H]2C(=O)N2C[C@H](c3ccccc3)CC23CC3)C1)[C@H]1C[C@@H](C(F)(F)F)C1 ; O=C(Oc1c(F)c(F)c(F)c(F)c1F)c1cc2cc([C@@H](F)P(=O)(O)O)ccc2s1 ; CN([C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1)[C@H]1C[C@@H](C(F)(F)F)C1]

To a solution of (3S,6S,9S,10aR)-6-amino-9-(methyl((1s,3R)-3-(trifluoromethyl)cyclobutyl)amino)-3-((S)-6-phenyl-4-azaspiro[2.4]heptane-4-carbonyl)octahydropyrrolo[1,2-a]azocin-5(1H)-one (2, 30 mg, TFA salt) in DMF (2 mL) was added TEA (9.6 mg, 0.095 mmol, 2 eq.), (S)-(fluoro(2-((perfluorophenoxy)carbonyl)benzo[b]thiophen-5-yl)methyl)phosphonic acid (3, 21.6 mg, 0.047 mmol, 1 eq.). The resulting mixture was stirred for 1 h at room temperature then purified by Prep-HPLC to afford ((S)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A65, 23 mg) as a white solid. LCMS (ESI): m/z=805 [M+H]⁺. 1H NMR (400 MHz, CD₃OD-d₄) δ (ppm) 8.08 (d, J=8.7 Hz, 2H), 7.89 (d, J=8.4 Hz, 1H), 7.61 (d, J=8.4 Hz, 1H), 7.36-7.15 (m, 5H), 5.79-5.50 (m, 1H), 4.71-4.50 (m, 2H), 4.48-4.31 (m, 1H), 4.10-3.65 (m, 4H), 3.57-3.46 (m, 1H), 2.99-2.50 (m, 6H), 2.45-1.55 (m, 16H), 0.60-0.35 (m, 2H). ¹⁹F NMR (376 MHz, CD₃OD-d₄) δ (ppm) −74.43 (s), −196.02 (d, J=76.0 Hz). ³¹P NMR (162 MHz, CD₃OD-d₄) δ (ppm) 9.65 (d, J=76.0 Hz).

### Step C: propyl ((S)-((S)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

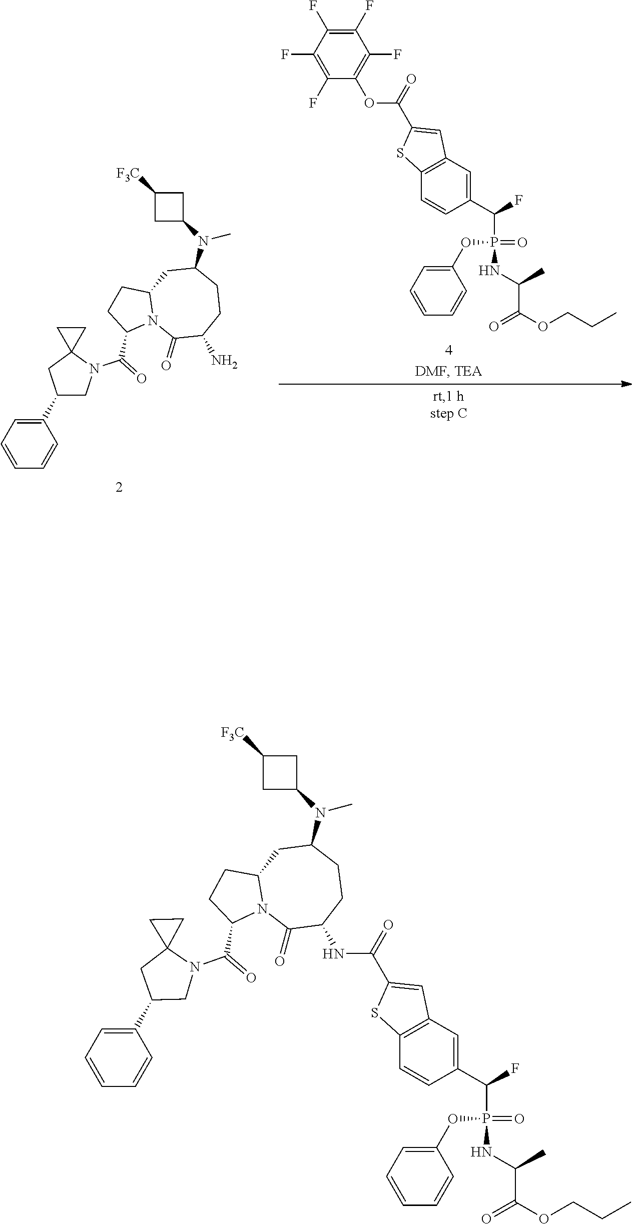 [SMILES: CN([C@H]1CC[C@H](N)C(=O)N2[C@H](CC[C@H]2C(=O)N2C[C@H](c3ccccc3)CC23CC3)C1)[C@H]1C[C@@H](C(F)(F)F)C1 ; CCCOC(=O)[C@H](C)N[P@@](=O)(Oc1ccccc1)[C@H](F)c1ccc2sc(C(=O)Oc3c(F)c(F)c(F)c(F)c3F)cc2c1 ; CCCOC(=O)[C@H](C)N[P@@](=O)(Oc1ccccc1)[C@H](F)c1ccc2sc(C(=O)N[C@H]3CC[C@H](N(C)[C@H]4C[C@@H](C(F)(F)F)C4)C[C@H]4CC[C@@H](C(=O)N5C[C@H](c6ccccc6)CC56CC6)N4C3=O)cc2c1]

To a solution of (3S,6S,9S,10aR)-6-amino-9-(methyl((1s,3R)-3-(trifluoromethyl)cyclobutyl)amino)-3-((S)-6-phenyl-4-azaspiro[2.4]heptane-4-carbonyl)octahydropyrrolo[1,2-a]azocin-5(1H)-one (2, 30 mg, TFA salt) in DMF (2 mL) was added TEA (9.6 mg, 0.095 mmol, 2 eq.), (perfluorophenyl 5-((S)-fluoro((S)-(((S)-1-oxo-1-propoxypropan-2-yl)amino)(phenoxy)phosphoryl)methyl)benzo[b]thiophene-2-carboxylate (4, 30.6 mg, 0.047 mmol, 1 eq.). The resulting mixture was stirred for 1 h at room temperature, then the crude product was purified by Prep-HPLC to afford propyl ((S)-((S)-fluoro(2-(((3S,6S,9S,10aR)-9-(methyl((1s,3R)-3-(trifluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C210, 13 mg) as a white solid. LCMS (ESI): m/z=994 [M+H]⁺. 1H NMR (400 MHz, DMSO-d₆) δ (ppm) 8.77 (d, J=7.6 Hz, 1H), 8.28 (s, 1H), 8.13-7.99 (m, 2H), 7.61 (d, J=8.5 Hz, 1H), 7.39-7.26 (m, 6H), 7.24-7.13 (m, 4H), 6.24-6.06 (m, 2H), 4.78-4.64 (m, 1H), 4.48 (t, J=8.6 Hz, 1H), 4.36-4.23 (m, 1H), 4.11 (t, J=8.6 Hz, 1H), 3.96-3.85 (m, 2H), 3.77-3.63 (m, 2H), 3.59-3.46 (m, 1H), 3.07-2.90 (m, 2H), 2.84-2.69 (m, 1H), 2.38-2.17 (m, 3H), 2.14-2.03 (m, 3H), 2.02-1.90 (m, 6H), 1.86-1.44 (m, 11H), 0.94 (d, J=7.1 Hz, 3H), 0.81 (t, J=7.4 Hz, 3H), 0.54-0.37 (m, 2H). ¹⁹F NMR (376 MHz, DMSO-d₆) δ (ppm) −71.26 (s), −194.18 (d, J=82.9 Hz). ³¹P NMR (162 MHz, DMSO-d₆) δ (ppm) 16.94 (d, J=82.8 Hz).

### Synthesis of ((R)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A37)

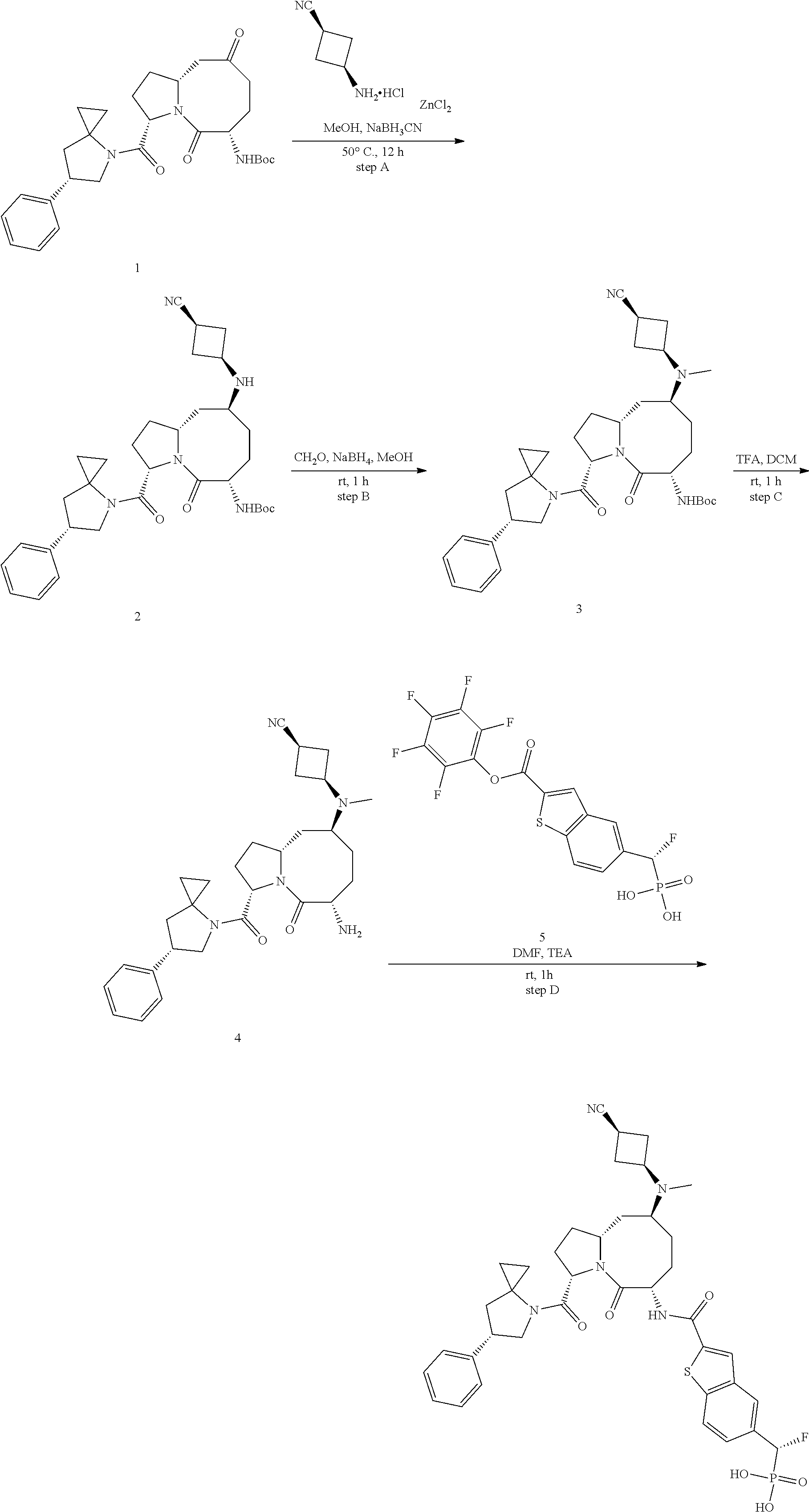

### Step A: tert-butyl ((3S,6S,9S,10aR)-9-(((1s,3R)-3-cyanocyclobutyl)amino)-5-oxo-3-((S)-6-phenyl-4-azaspiro[2.4]heptane-4-carbonyl)decahydropyrrolo[1,2-a]azocin-6-yl)carbamate

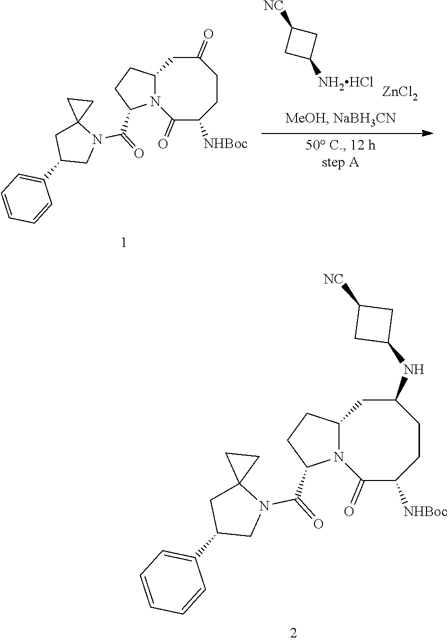

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (1, 1 g, 2.02 mmol, 1 eq.) in MeOH (20 mL) was added (1s,3s)-3-aminocyclobutane-1-carbonitrile hydrochloride (400 mg, 3.03 mml, 1.5 eq.), ZnCl₂ (2.75 g, 20.2 mmol, 10 eq.). The resulting mixture was stirred for 1 h at 50° C., then NaBH₃CN (1.27 g, 20.2 mmol, 10 eq.) was added portion wise every 30 mins (2 eq. (254 mg each time) added 5 times). The mixture was stirred for 12 h at 50° C., then cooled to room temperature, and used to the next step directly without further workup. LCMS (ESI): m/z=576 [M+H]⁺.

### Step B: tert-butyl ((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamate

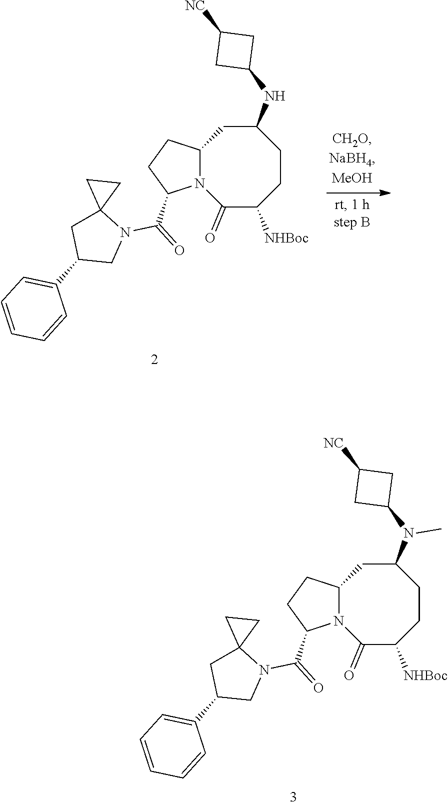 [SMILES: CC(C)(C)OC(=O)N[C@H]1CC[C@H](N[C@H]2C[C@@H](C#N)C2)C[C@H]2CC[C@@H](C(=O)N3C[C@H](c4ccccc4)CC34CC4)N2C1=O ; CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](C#N)C1]

To the above mixture (step A) was added 40% formaldehyde (1 mL) at room temperature, the mixture was stirred for 10 min, then NaBH₄ (764 mg, 20.2 mmol, 10 eq.) was added to the mixture, stirred for another 1 hr. The reaction was quenched by NH₄Cl (sat. aq), diluted with H₂O (100 mL), then extracted with EtOAc (50 mL×3), the organic solution was dried over Na₂SO₄, then concentrated under reduced pressure, the crude product was purified by C18 column (MeCN:H₂O (0.1% ammonium hydroxide)=5%-70%) to afford tert-butyl ((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamate (3, 1.1 g, 1.87 mmol, 92% yield, two steps) as a white solid. LCMS (ESI): m/z=590 [M+H]⁺.

### Step C: (1R,3s)-3-(((3S,6S,9S,10aR)-6-amino-5-oxo-3-((S)-6-phenyl-4-azaspiro[2.4]heptane-4-carbonyl)decahydropyrrolo[1,2-a]azocin-9-yl)(methyl)amino)cyclobutane-1-carbonitrile

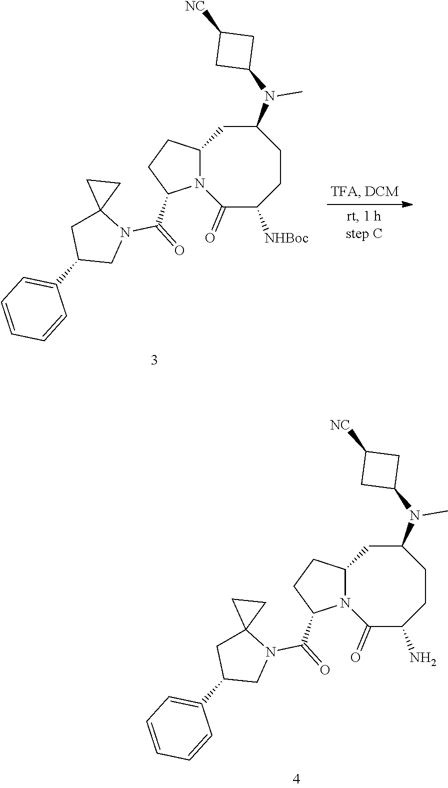 [SMILES: CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](C#N)C1 ; CN([C@H]1CC[C@H](N)C(=O)N2[C@H](CC[C@H]2C(=O)N2C[C@H](c3ccccc3)CC23CC3)C1)[C@H]1C[C@@H](C#N)C1]

To a solution of tert-butyl ((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamate (3, 30 mg, 0.051 mmol) in DCM (2 mL) was added TFA (2 mL), The resulting mixture was stirred for 1 h at room temperature, and the resulting mixture was concentrated to dryness under reduced pressure to afford crude product (1R,3s)-3-(((3S,6S,9S,10aR)-6-amino-5-oxo-3-((S)-6-phenyl-4-azaspiro[2.4]heptane-4-carbonyl)decahydropyrrolo[1,2-a]azocin-9-yl)(methyl)amino)cyclobutane-1-carbonitrile (4, 30 mg, TFA salt) as a yellow oil which used in the next step without further purification. LCMS (ESI): m/z=490 [M+H]⁺.

### Step D: ((R)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid

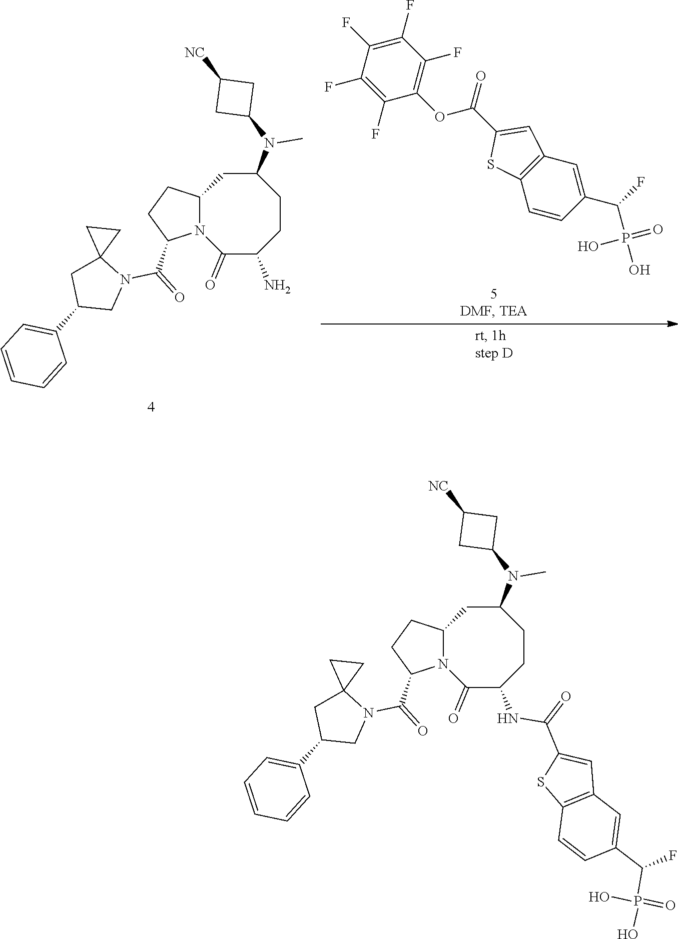 [SMILES: CN([C@H]1CC[C@H](N)C(=O)N2[C@H](CC[C@H]2C(=O)N2C[C@H](c3ccccc3)CC23CC3)C1)[C@H]1C[C@@H](C#N)C1 ; O=C(Oc1c(F)c(F)c(F)c(F)c1F)c1cc2cc([C@H](F)P(=O)(O)O)ccc2s1 ; CN([C@H]1CC[C@H](NC(=O)c2cc3cc([C@H](F)P(=O)(O)O)ccc3s2)C(=O)N2[C@H](CC[C@H]2C(=O)N2C[C@H](c3ccccc3)CC23CC3)C1)[C@H]1C[C@@H](C#N)C1]

To a solution of (1R,3s)-3-(((3S,6S,9S,10aR)-6-amino-5-oxo-3-((S)-6-phenyl-4-azaspiro[2.4]heptane-4-carbonyl)decahydropyrrolo[1,2-a]azocin-9-yl)(methyl)amino)cyclobutane-1-carbonitrile (4, 30 mg, TFA salt) in DMF (2 mL) was added TEA (10.3 mg, 0.102 mmol, 2 eq.), (R)-(fluoro(2-((perfluorophenoxy)carbonyl)benzo[b]thiophen-5-yl)methyl)phosphonic acid (5, 23.2 mg, 0.051 mmol, 1 eq). The resulting mixture was stirred for 1 h at room temperature. The crude product was purified by Prep-HPLC to afford propyl ((R)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A37, 11 mg, 0.015 mmol) as a white solid. LCMS (ESI): m/z=762 [M+H]⁺. 1H NMR (400 MHz, CD₃OD-d₄) δ (ppm) 8.15-8.08 (m, 1H), 8.07-8.01 (m, 1H), 8.00-7.92 (m, 1H), 7.66-7.54 (m, 1H), 7.42-7.15 (m, 5H), 6.06-5.68 (m, 1H), 4.91-4.80 (m, 1H), 4.73-4.63 (m, 1H), 4.62-4.50 (m, 1H), 4.20-3.79 (m, 4H), 3.69-3.47 (m, 1H), 3.24-3.10 (m, 1H), 3.01-2.73 (m, 3H), 2.73-2.66 (m, 3H), 2.58-2.45 (m, 1H), 2.43-1.62 (m, 13H), 0.67-0.53 (m, 2H). ¹⁹F NMR (377 MHz, CD₃OD-d₄) δ (ppm) −199.44 (d, J=82.9 Hz). ³¹P NMR (162 MHz, CD₃OD-d₄) δ (ppm) 13.11 (d, J=85.9 Hz).

### Synthesis of propyl (((R)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(2,2,2-trifluoroethoxy)phosphoryl)-L-alaninate (Compound C110)

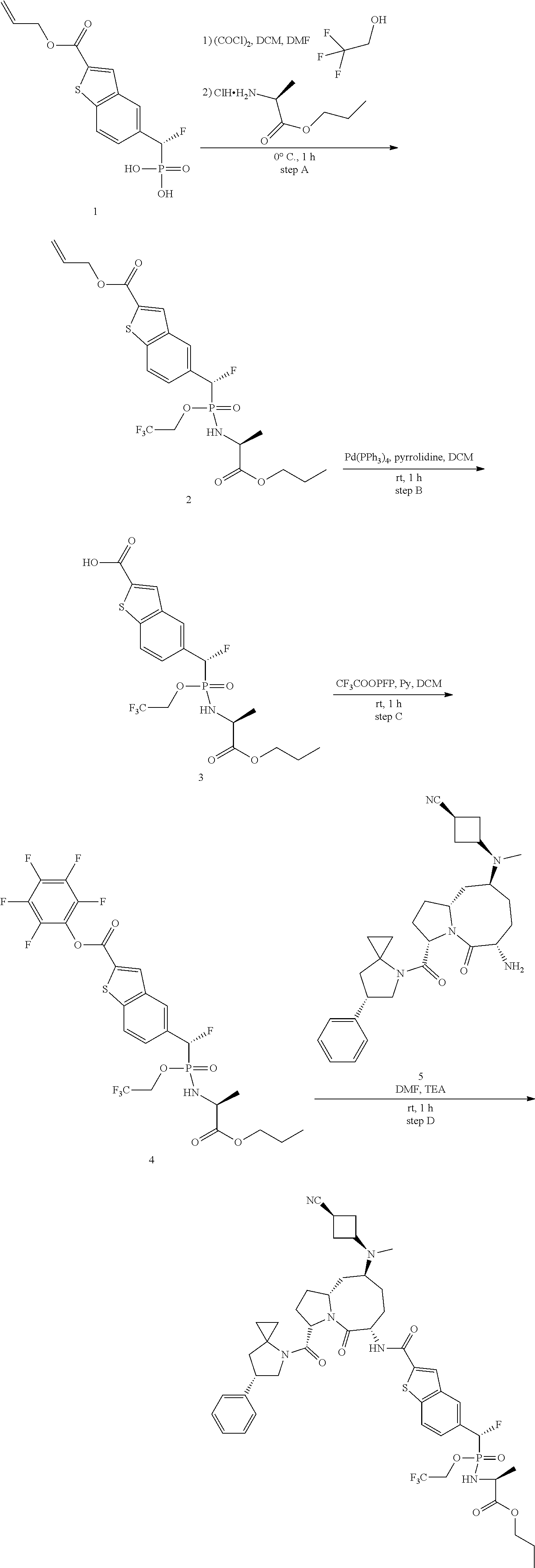

### Step A: allyl 5-((1R)-fluoro((((S)-1-oxo-1-propoxypropan-2-yl)amino)(2,2,2-trifluoroethoxy)phosphoryl)methyl)benzo[b]thiophene-2-carboxylate

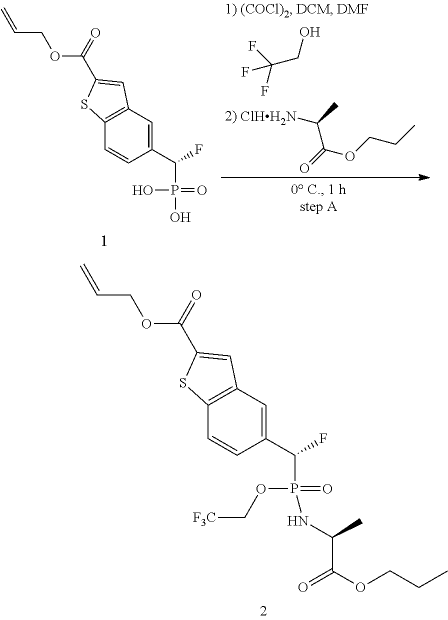

To a solution of (R)-((2-((allyloxy)carbonyl)benzo[b]thiophen-5-yl)fluoromethyl)phosphonic acid (1, 2.6 g, 7.88 mmol, 1 eq.) in DCM (40 mL) was added DMF (1 drop) and oxalyl chloride (5 g, 39.4 mmol, 5 eq). The resulting mixture was stirred for 1 h at room temperature, then dried under reduced pressure. The residue was dissolved in DCM (40 mL) and cooled to 0° C., 2,2,2-trifluoroethan-1-ol (788 mg, 7.88 mmol, 1 eq) was added to the solution, then TEA (4 g, 39.4 mmol, 5 eq.) was added dropwise to the solution, the mixture was stirred for 1 h at 0° C. and propyl L-alaninate hydrochloride (2.64 g, 15.76 mmol, 2 eq.) was added to the solution, then stirred for 10 min. The reaction was quenched by adding H₂O (50 mL), then extracted with DCM (50 mL×2), the organic layers were combined and washed with brine, then dried over Na₂SO₄, concentrated to dryness under reduced pressure The crude product was purified by silica gel column chromatography to afford allyl 5-((1R)-fluoro((((S)-1-oxo-1-propoxypropan-2-yl)amino)(2,2,2-trifluoroethoxy)phosphoryl)methyl)benzo[b]thiophene-2-carboxylate (2, 7.72 g, 3.28 mmol, 41.6% yield) as a colorless oil. LCMS (ESI): m/z=526 [M+H]⁺.

### Step B: 5-((1R)-fluoro((((S)-1-oxo-1-propoxypropan-2-yl)amino)(2,2,2-trifluoroethoxy)phosphoryl)methyl)benzo[b]thiophene-2-carboxylic acid

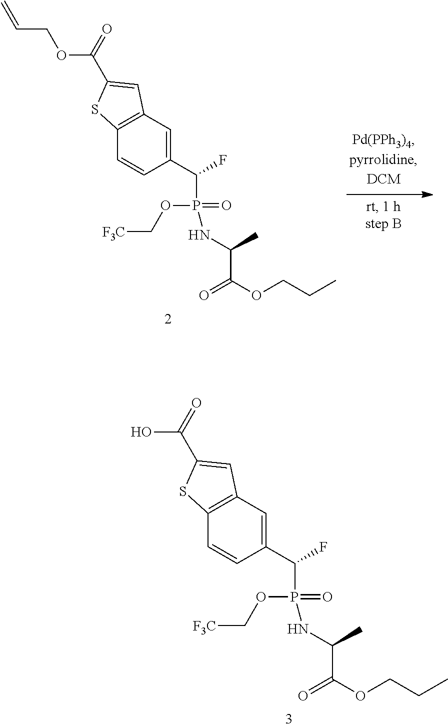 [SMILES: C=CCOC(=O)c1cc2cc([C@H](F)P(=O)(N[C@@H](C)C(=O)OCCC)OCC(F)(F)F)ccc2s1 ; CCCOC(=O)[C@H](C)NP(=O)(OCC(F)(F)F)[C@@H](F)c1ccc2sc(C(=O)O)cc2c1]

To a solution of allyl 5-((1R)-fluoro((((S)-1-oxo-1-propoxypropan-2-yl)amino)(2,2,2-trifluoroethoxy)phosphoryl)methyl)benzo[b]thiophene-2-carboxylate (2, 860 mg, 1.64 mmol, 1 eq.) in DCM (10 mL) was added Pd(PPh₃)₄ (184 mg, 0.16 mmol, 0.1 eq.), pyrrolidine (116 mg, 1.64 mmol, 1 eq.). The resulting mixture was stirred for 1 h at room temperature under the atmosphere of N₂, The pH was adjusted to pH=3 with 1M HCl (aq.), then extracted with DCM (20 mL×2), the organic layers were combined and washed with brine (20 mL) dried over Na₂SO₄, The resulting mixture was concentrated to dryness under reduced pressure to afford crude product 5-((1R)-fluoro((((S)-1-oxo-1-propoxypropan-2-yl)amino)(2,2,2-trifluoroethoxy)phosphoryl)methyl)benzo[b]thiophene-2-carboxylic acid (3, 650 mg, 1.34 mmol, 81.7% yield) as a yellow solid. LCMS (ESI): m/z=486 [M+H]⁺.

### Step C: perfluorophenyl 5-((1R)-fluoro((((S)-1-oxo-1-propoxypropan-2-yl)amino)(2,2,2-trifluoroethoxy)phosphoryl)methyl)benzo[b]thiophene-2-carboxylate

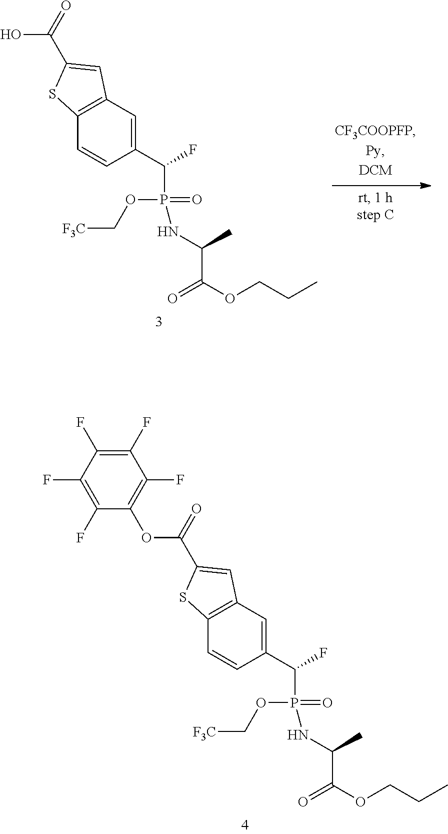 [SMILES: CCCOC(=O)[C@H](C)NP(=O)(OCC(F)(F)F)[C@@H](F)c1ccc2sc(C(=O)O)cc2c1 ; CCCOC(=O)[C@H](C)NP(=O)(OCC(F)(F)F)[C@@H](F)c1ccc2sc(C(=O)Oc3c(F)c(F)c(F)c(F)c3F)cc2c1]

To a solution of 5-((1R)-fluoro((((S)-1-oxo-1-propoxypropan-2-yl)amino)(2,2,2-trifluoroethoxy)phosphoryl)methyl)benzo[b]thiophene-2-carboxylic acid (3, 650 mg, 1.34 mmol, 1 eq.) in DCM (10 mL) was added pyridine (317 mg, 4.02 mmol, 3 eq.), perfluorophenyl 2,2,2-trifluoroacetate (750 mg, 2.68 mmol, 2 eq.). The resulting mixture was stirred for 1 hr at room temperature, then. H₂O (20 mL) was added to the solution, then extracted with DCM (30 mL×3), the organic layers were combined and washed with brine (30 mL), dried over Na₂SO₄, then concentrated under vacuum. The crude product was purified by silica gel column chromatography to afford perfluorophenyl 5-((1R)-fluoro((((S)-1-oxo-1-propoxypropan-2-yl)amino)(2,2,2-trifluoroethoxy)phosphoryl)methyl)benzo[b]thiophene-2-carboxylate (4, 600 mg, 0.92 mmol, 67% yield) as a white solid. LCMS (ESI): m/z=652 [M+H]⁺.

### Step D: propyl (((R)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(2,2,2-trifluoroethoxy)phosphoryl)-L-alaninate

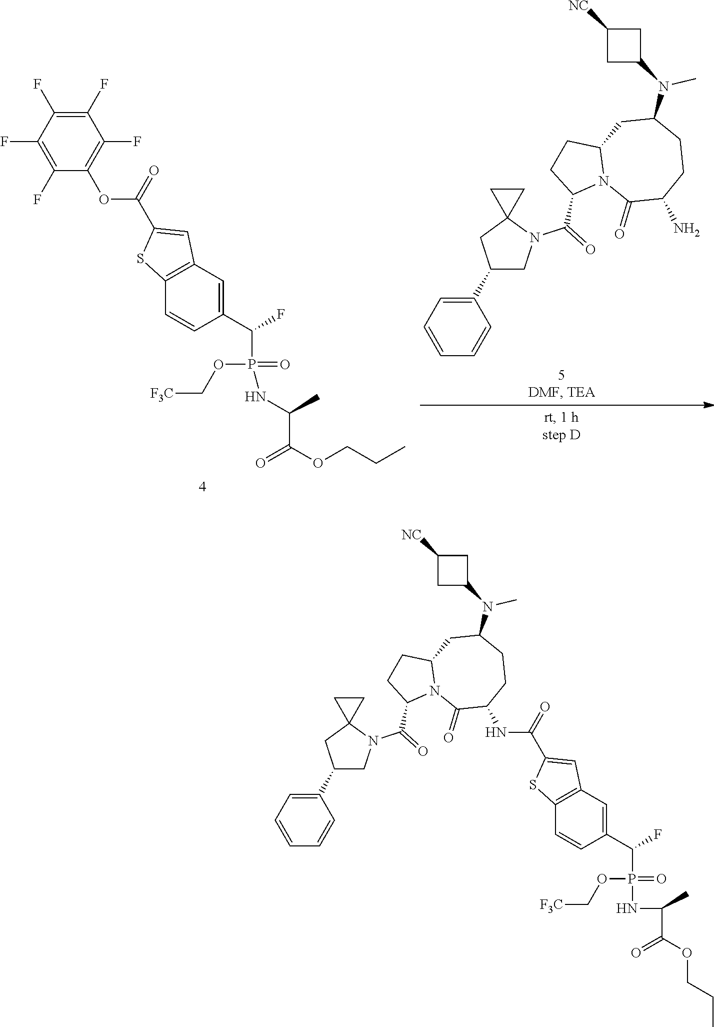

To a solution of perfluorophenyl 5-((1R)-fluoro((((S)-1-oxo-1-propoxypropan-2-yl)amino)(2,2,2-trifluoroethoxy)phosphoryl)methyl)benzo[b]thiophene-2-carboxylate (4, 30 mg, 0.046 mml, 1 eq.) in DMF (3 mL) was added (1R,3s)-3-(((3S,6S,9S,10aR)-6-amino-5-oxo-3-((S)-6-phenyl-4-azaspiro[2.4]heptane-4-carbonyl)decahydropyrrolo[1,2-a]azocin-9-yl)(methyl)amino)cyclobutane-1-carbonitrile (5, 22.5 mg, 0.046 mmol, 1 eq.), TEA (14 mg, 0.138 mmol, 3 eq). The resulting mixture was stirred for 1 hr at room temperature, then the crude product was purified by Prep-HPLC to afford propyl (((R)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(2,2,2-trifluoroethoxy)phosphoryl)-L-alaninate (Compound C110, 16 mg, 0.017 mmol) as a white solid. LCMS (ESI): m/z=957 [M+H]⁺. ¹H NMR (400 MHz, DMSO-d₆) δ (ppm) 9.94-9.42 (m, 1H), 8.99-8.71 (m, 1H), 8.33-8.16 (m, 1H), 8.14-7.93 (m, 2H), 7.67-7.47 (m, 1H), 7.42-7.27 (m, 4H), 7.26-7.14 (m, 1H), 6.38-5.86 (m, 2H), 4.86-4.75 (m, 1H), 4.66-4.47 (m, 4H), 4.20-4.09 (m, 2H), 4.04-3.89 (m, 4H), 3.87-3.77 (m, 2H), 3.75-3.62 (m, 2H), 3.59-3.51 (m, 1H), 3.27-3.13 (m, 1H), 2.74-2.62 (m, 2H), 2.61-2.53 (m, 4H), 2.49-2.42 (m, 1H), 2.29-2.19 (m, 1H), 2.15-1.97 (m, 5H), 1.90-1.74 (m, 4H), 1.60-1.45 (m, 3H), 1.32-1.16 (m, 3H), 0.91-0.77 (m, 3H), 0.66-0.38 (m, 2H). ¹⁹F NMR (377 MHz, DMSO-d₆) δ (ppm) −72.97-−75.39 (m), −193.72-−197.54 (m). ³¹P NMR (162 MHz, DMSO-d₆) δ (ppm) 22.10-20.38 (m).

### Synthesis of ((S)-(2-(((3S,6S,9S,10aR)-9-(((S)-3,3-difluorocyclopentyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A36) and propyl (((S)-(2-(((3S,6S,9S,10aR)-9-(((S)-3,3-difluorocyclopentyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(2,2,2-trifluoroethoxy)phosphoryl)-L-alaninate (Compound C95)

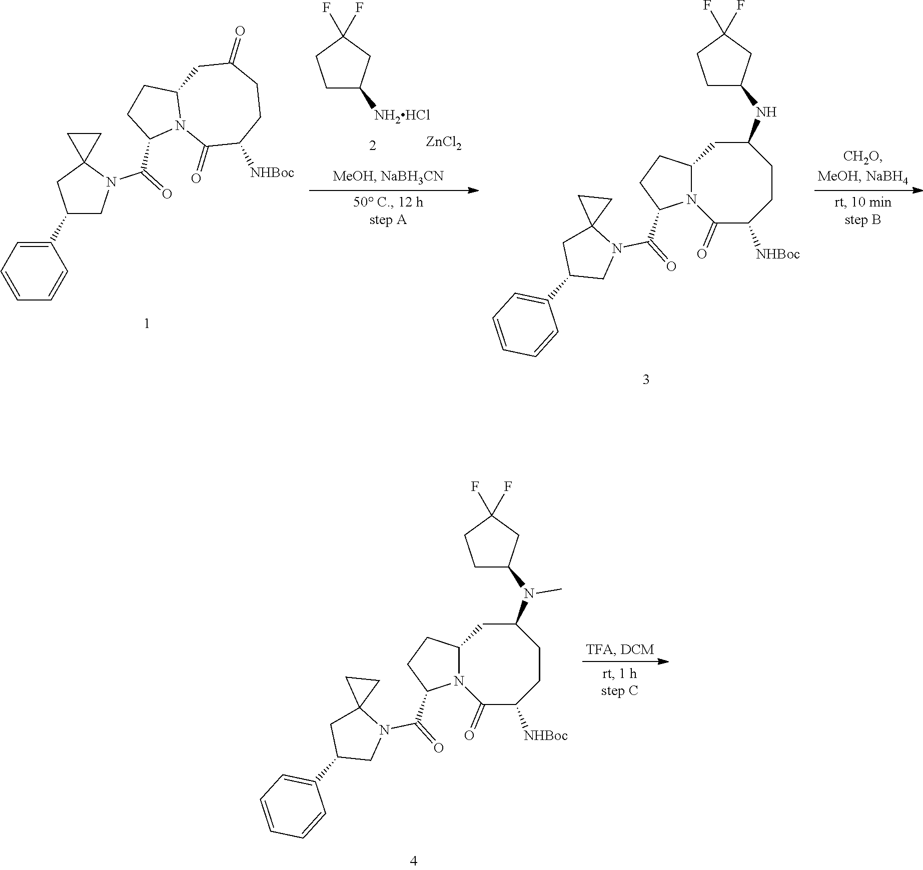

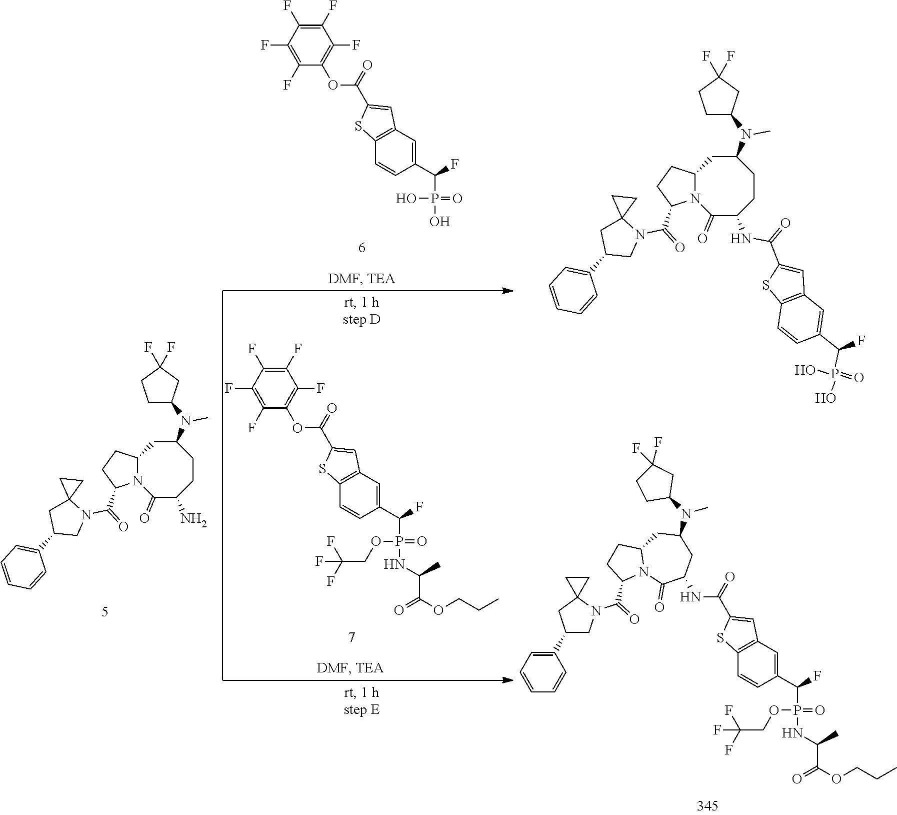

Step A: tert-butyl ((3S,6S,9S,10aR)-9-(((S)-3,3-difluorocyclopentyl)amino)-5-oxo-3-((S)-6-phenyl-4-azaspiro[2.4]heptane-4-carbonyl)decahydropyrrolo[1,2-a]azocin-6-yl)carbamate

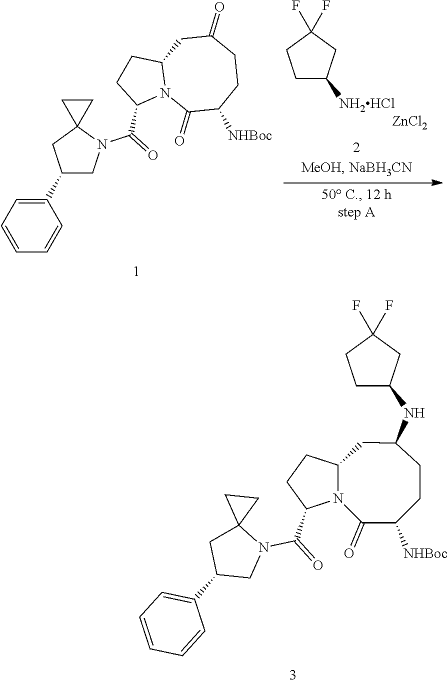

To a solution of tert-butyl ((3S,6S,10aR)-5,9-dioxo-3-((R)-6-phenyl-4-azaspiro[2.4]heptane-4-carbonyl)decahydropyrrolo[1,2-a]azocin-6-yl)carbamate (1, 1 g, 2.02 mmol, 1 eq.) in MeOH (20 mL) was added (1s,3s)-3-(trifluoromethyl)cyclobutan-1-amine hydrochloride (2, 477 mg, 3.03 mmol, 1.5 eq.), ZnCl₂ (2.77 g, 20.2 mmol, 10 eq.), the resulting mixture was stirred for 2 hr. at 50° C. under the atmosphere of N₂. Then NaBH₃CN (1.27 g, 20.2 mmol, 10 eq.) was added portion wise every 30 mins (2 eq. (254 mg each time) added 5 times). The mixture was stirred for 12 hr at 50° C., then cooled to room temperature and used to next step without further workup. LCMS (ESI): m/z=601 [M+H]⁺.

### Step B: tert-butyl ((3S,6S,9S,10aR)-9-(((S)-3,3-difluorocyclopentyl)(methyl)amino)-5-oxo-3-((S)-6-phenyl-4-azaspiro[2.4]heptane-4-carbonyl)decahydropyrrolo[1,2-a]azocin-6-yl)carbamate

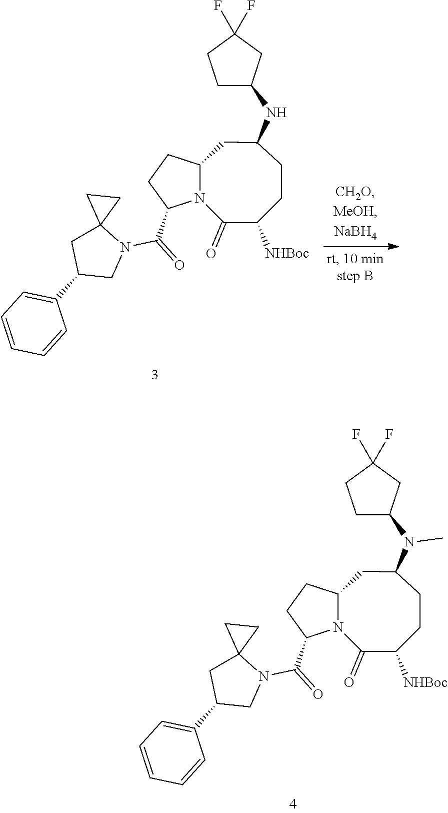 [SMILES: CC(C)(C)OC(=O)N[C@H]1CC[C@H](N[C@H]2CCC(F)(F)C2)C[C@H]2CC[C@@H](C(=O)N3C[C@H](c4ccccc4)CC34CC4)N2C1=O ; CN([C@H]1CC[C@H](NC(=O)OC(C)(C)C)C(=O)N2[C@H](CC[C@H]2C(=O)N2C[C@H](c3ccccc3)CC23CC3)C1)[C@H]1CCC(F)(F)C1]

To the above mixture (step A) was added 40% formaldehyde aqueous (3 mL) at room temperature, the mixture was stirred for 30 min, then NaBH₄ (383 mg, 10.1 mmol, 5 eq.) was added in portions, the mixture was stirred for another 30 min. The reaction was quenched by NH₄Cl (sat. aq), diluted with H₂O (80 mL), then extracted with EtOAc (40 mL×3), the organic layers were combined and washed with brine (20 mL), dried over Na₂SO₄, then concentrated to dryness under reduced pressure. The crude product was purified by C18 column (MeCN:H₂O (0.1% ammonium hydroxide)=5%-70%) to afford tert-butyl ((3S,6S,9S,10aR)-9-(((S)-3,3-difluorocyclopentyl)(methyl)amino)-5-oxo-3-((S)-6-phenyl-4-azaspiro[2.4]heptane-4-carbonyl)decahydropyrrolo[1,2-a]azocin-6-yl)carbamate (4, 700 mg, 1.14 mmol, 56.4% yield two steps) as a white solid. LCMS (ESI): m/z=615 [M+H]⁺.

### Step C: (3S,6S,9S,10aR)-6-amino-9-(((S)-3,3-difluorocyclopentyl)(methyl)amino)-3-((S)-6-phenyl-4-azaspiro[2.4]heptane-4-carbonyl)octahydropyrrolo[1,2-a]azocin-5(1H)-one

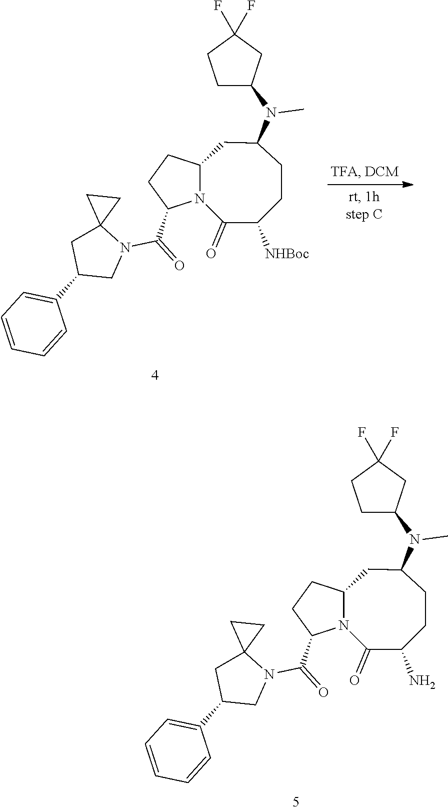 [SMILES: CN([C@H]1CC[C@H](NC(=O)OC(C)(C)C)C(=O)N2[C@H](CC[C@H]2C(=O)N2C[C@H](c3ccccc3)CC23CC3)C1)[C@H]1CCC(F)(F)C1 ; CN([C@H]1CC[C@H](N)C(=O)N2[C@H](CC[C@H]2C(=O)N2C[C@H](c3ccccc3)CC23CC3)C1)[C@H]1CCC(F)(F)C1]

To a solution of tert-butyl ((3S,6S,9S,10aR)-9-(((S)-3,3-difluorocyclopentyl)(methyl)amino)-5-oxo-3-((S)-6-phenyl-4-azaspiro[2.4]heptane-4-carbonyl)decahydropyrrolo[1,2-a]azocin-6-yl)carbamate (4, 30 mg, 0.047 mmol) in DCM (2 mL) was added TFA (2 mL). The resulting mixture was stirred for 1 h at room temperature then the resulting mixture was concentrated to dryness under reduced pressure to afford crude product (3S,6S,9S,10aR)-6-amino-9-(((S)-3,3-difluorocyclopentyl)(methyl)amino)-3-((S)-6-phenyl-4-azaspiro[2.4]heptane-4-carbonyl)octahydropyrrolo[1,2-a]azocin-5(1H)-one (5, 30 mg, TFA salt) as a yellow oil which was used to the next step without further purification. LCMS (ESI): m/z=515 [M+H]⁺.

### Step D: ((S)-(2-(((3S,6S,9S,10aR)-9-(((S)-3,3-difluorocyclopentyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid

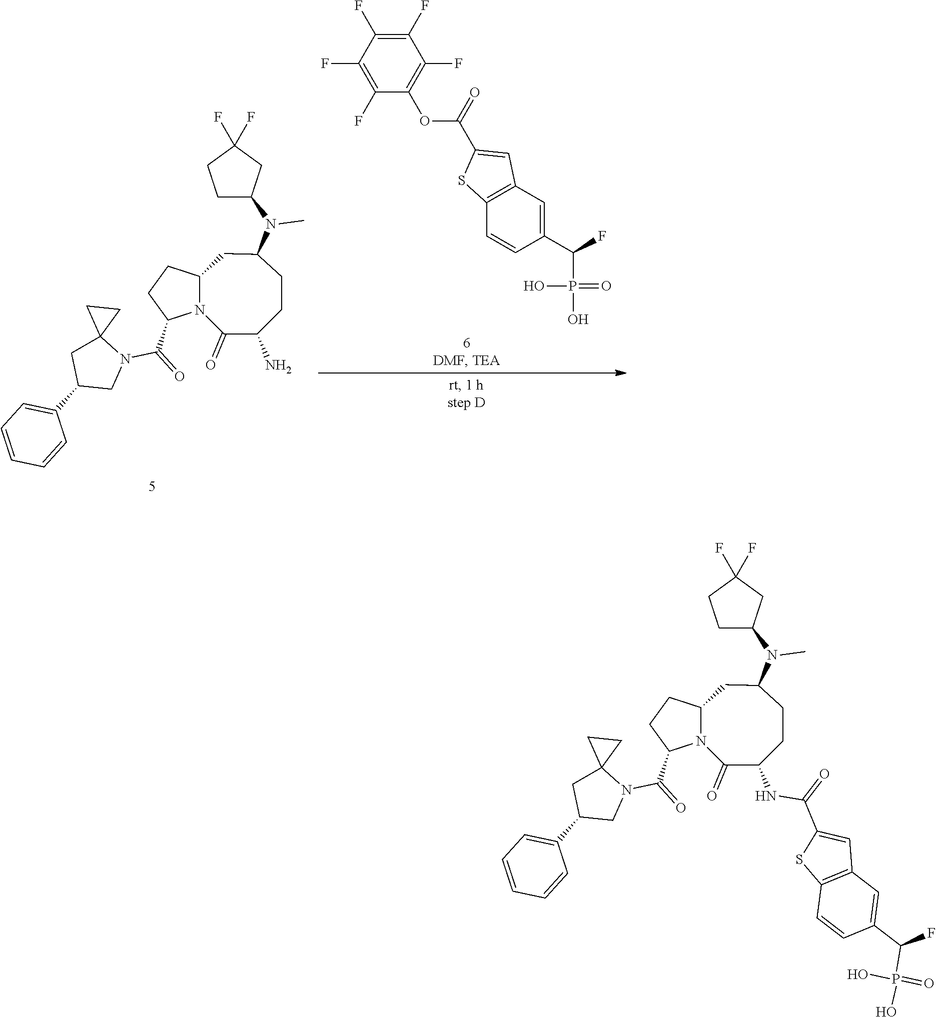 [SMILES: CN([C@H]1CC[C@H](N)C(=O)N2[C@H](CC[C@H]2C(=O)N2C[C@H](c3ccccc3)CC23CC3)C1)[C@H]1CCC(F)(F)C1 ; O=C(Oc1c(F)c(F)c(F)c(F)c1F)c1cc2cc([C@@H](F)P(=O)(O)O)ccc2s1 ; CN([C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1)[C@H]1CCC(F)(F)C1]

To a solution of (3S,6S,9S,10aR)-6-amino-9-(((S)-3,3-difluorocyclopentyl)(methyl)amino)-3-((S)-6-phenyl-4-azaspiro[2.4]heptane-4-carbonyl)octahydropyrrolo[1,2-a]azocin-5(1H)-one (5, 30 mg, TFA salt) in DMF (2 mL) was added TEA (9.9 mg, 0.098 mmol, 2 eq.), (S)-(fluoro(2-((perfluorophenoxy)carbonyl)benzo[b]thiophen-5-yl)methyl)phosphonic acid (6, 22.3 mg, 0.049 mmol, 1 eq.). The resulting mixture was stirred for 1 h at room temperature, LCMS show that the reaction was completed. The crude product was purified by Prep-HPLC to afford ((S)-(2-(((3S,6S,9S,10aR)-9-(((S)-3,3-difluorocyclopentyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A36, 8 mg) as a white solid. LCMS (ESI): m/z=787 [M+H]⁺. ¹H NMR (400 MHz, CD₃OD-d₄) δ (ppm) 8.09 (d, J=5.4 Hz, 1H), 8.03 (s, 1H), 7.98-7.89 (m, 1H), 7.58 (d, J=8.4 Hz, 1H), 7.38-7.25 (m, 4H), 7.24-7.17 (m, 1H), 5.93-5.71 (m, 1H), 4.71-4.54 (m, 2H), 4.39-4.03 (m, 2H), 3.98-3.83 (m, 2H), 3.67-3.32 (m, 2H), 2.84-2.76 (m, 3H), 2.55-1.85 (m, 19H), 1.73-1.58 (m, 1H), 0.62-0.43 (m, 2H). 1⁹F NMR (376 MHz, CD₃OD-d₄) δ (ppm) −89.99 (d, J=12.3 Hz), −90.74-92.79 (m), −199.39 (dd, J=85.6, 5.4 Hz). ³¹P NMR (162 MHz, CD₃OD-d₄) δ (ppm) 13.07 (d, J=85.3 Hz).

### Step E: propyl (((S)-(2-(((3S,6S,9S,10aR)-9-(((S)-3,3-difluorocyclopentyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(2,2,2-trifluoroethoxy)phosphoryl)-L-alaninate

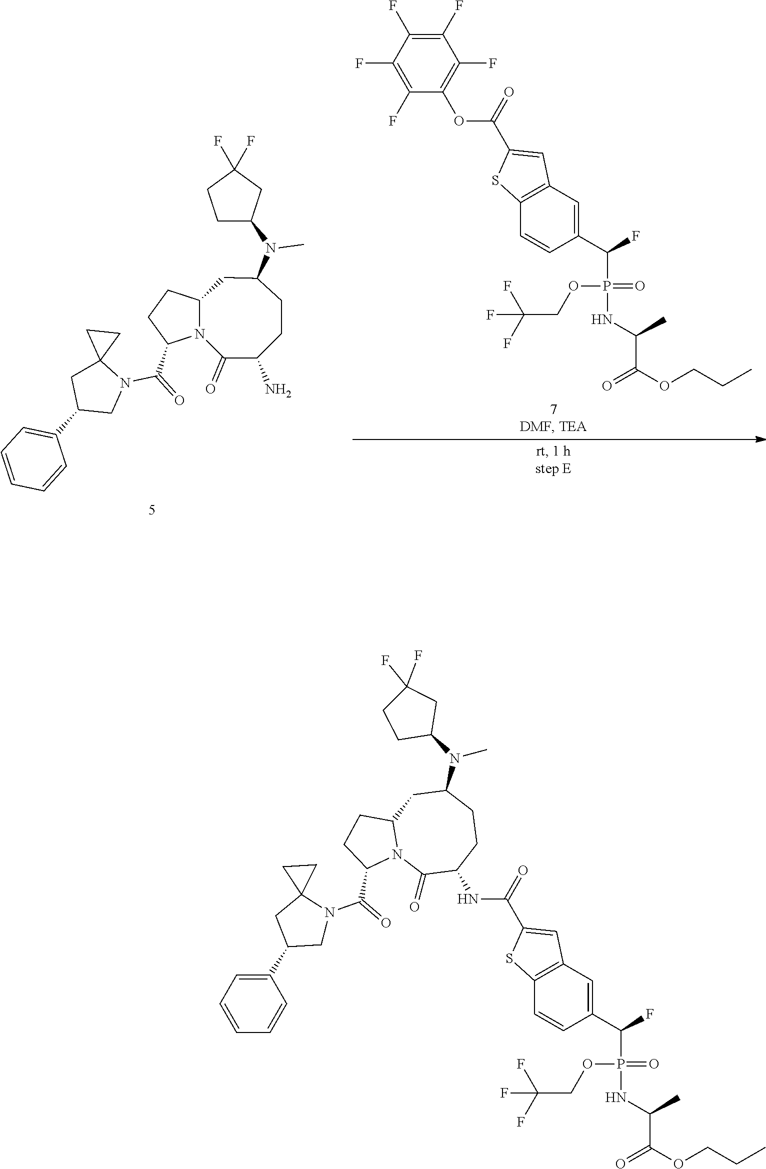

To a solution of (3S,6S,9S,10aR)-6-amino-9-(((S)-3,3-difluorocyclopentyl)(methyl)amino)-3-((S)-6-phenyl-4-azaspiro[2.4]heptane-4-carbonyl)octahydropyrrolo[1,2-a]azocin-5(1H)-one (5, 30 mg, TFA salt) in DMF (2 mL) was added TEA (9.9 mg, 0.098 mmol, 2 eq.), perfluorophenyl 5-((1S)-fluoro((((S)-1-oxo-1-propoxypropan-2-yl)amino)(2,2,2-trifluoroethoxy)phosphoryl)methyl)benzo[b]thiophene-2-carboxylate (7, 31.8 mg, 0.049 mmol, 1 eq.). The resulting mixture was stirred for 1 h at room temperature. The crude product was purified by Prep-HPLC to afford propyl (((S)-(2-(((3S,6S,9S,10aR)-9-(((S)-3,3-difluorocyclopentyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(2,2,2-trifluoroethoxy)phosphoryl)-L-alaninate (Compound C95, 11 mg) as a white solid. LCMS (ESI): m/z=982 [M+H]⁺. ¹H NMR (400 MHz, DMSO-d₆) δ (ppm) 8.85-8.71 (m, 1H), 8.31-8.22 (m, 1H), 8.11-7.94 (m, 2H), 7.56-7.46 (m, 1H), 7.37-7.28 (m, 4H), 7.24-7.15 (m, 1H), 6.28-5.90 (m, 2H), 4.79-4.68 (m, 1H), 4.66-4.41 (m, 3H), 4.35-4.25 (m, 1H), 4.14-3.62 (m, 5H), 3.58-3.47 (m, 1H), 3.11-2.93 (m, 2H), 2.72-2.58 (m, 1H), 2.40-2.32 (m, 1H), 2.25-2.06 (m, 6H), 2.02-1.68 (m, 12H), 1.64-1.48 (m, 5H), 1.30-1.00 (m, 3H), 0.89-0.81 (m, 3H), 0.52-0.38 (m, 2H). ¹⁹F NMR (376 MHz, DMSO-d₆) δ (ppm) −72.10-−76.44 (m), −83.74-−88.46 (m), −196.13 (dd, J=663.5, 84.9 Hz). ³¹P NMR (162 MHz, DMSO-d₆) δ (ppm) 21.33 (d, J=83.6 Hz), δ (ppm) 20.98 (d, J=86.2 Hz).

### Synthesis of ((S)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A40)

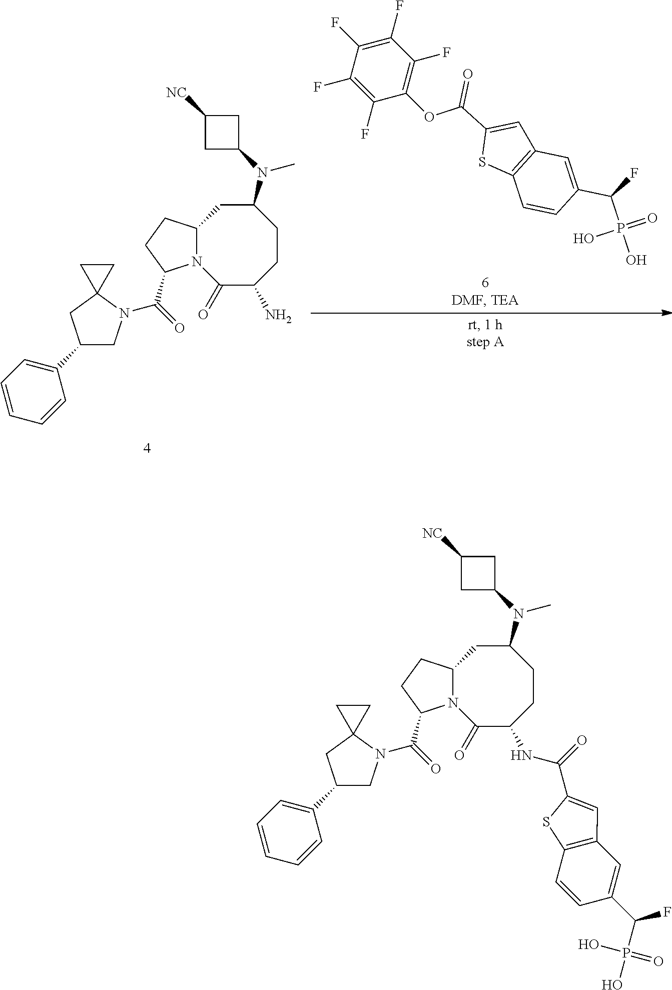 [SMILES: CN([C@H]1CC[C@H](N)C(=O)N2[C@H](CC[C@H]2C(=O)N2C[C@H](c3ccccc3)CC23CC3)C1)[C@H]1C[C@@H](C#N)C1 ; O=C(Oc1c(F)c(F)c(F)c(F)c1F)c1cc2cc([C@@H](F)P(=O)(O)O)ccc2s1 ; CN([C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1)[C@H]1C[C@@H](C#N)C1]

### Step A: ((S)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid

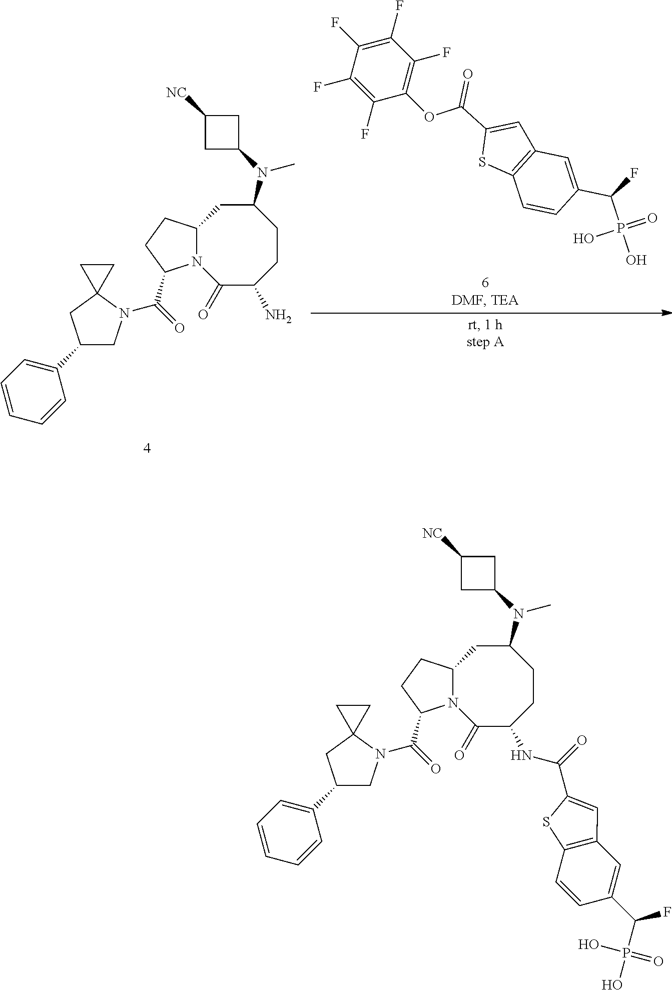 [SMILES: CN([C@H]1CC[C@H](N)C(=O)N2[C@H](CC[C@H]2C(=O)N2C[C@H](c3ccccc3)CC23CC3)C1)[C@H]1C[C@@H](C#N)C1 ; O=C(Oc1c(F)c(F)c(F)c(F)c1F)c1cc2cc([C@@H](F)P(=O)(O)O)ccc2s1 ; CN([C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1)[C@H]1C[C@@H](C#N)C1]

To a solution of (1R,3s)-3-(((3S,6S,9S,10aR)-6-amino-5-oxo-3-((S)-6-phenyl-4-azaspiro[2.4]heptane-4-carbonyl)decahydropyrrolo[1,2-a]azocin-9-yl)(methyl)amino)cyclobutane-1-carbonitrile (4, 30 mg, TFA salt) in DMF (2 mL) was added TEA (10.3 mg, 0.102 mmol, 2 eq.), (S)-(fluoro(2-((perfluorophenoxy)carbonyl)benzo[b]thiophen-5-yl)methyl)phosphonic acid (6, 23.2 mg, 0.051 mmol, 1 eq). The resulting mixture was stirred for 1 h at room temperature, LCMS show that the reaction was completed. The crude product was purified by Prep-HPLC to afford ((S)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A40, 16 mg, 0.021 mmol) as a white solid. LCMS (ESI): m/z=762 [M+H]⁺. 1H NMR (400 MHz, CD₃OD-d₄) δ (ppm) 8.04 (s, 2H), 7.86 (d, J=8.4 Hz, 1H), 7.63 (d, J=8.6 Hz, 1H), 7.40-7.26 (m, 4H), 7.23-7.16 (m, 1H), 5.75-5.52 (m, 1H), 4.78-4.71 (m, 2H), 4.64-4.52 (m, 2H), 4.46-4.35 (m, 1H), 4.12-3.88 (m, 2H), 3.65-3.52 (m, 1H), 3.02-2.86 (m, 1H), 2.70-2.57 (m, 1H), 2.55-2.15 (m, 8H), 2.11 (s, 3H), 2.02-1.93 (m, 3H), 1.88-1.79 (m, 2H), 1.78-1.61 (m, 4H), 0.57-0.46 (m, 2H). ¹⁹F NMR (377 MHz, CD₃OD-d₄) δ (ppm) −195.94 (d, J=76.2 Hz). ³¹P NMR (162 MHz, CD₃OD-d₄) δ (ppm) 9.91 (d, J=76.2 Hz).

### Synthesis of cyclobutyl (((S)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C112)

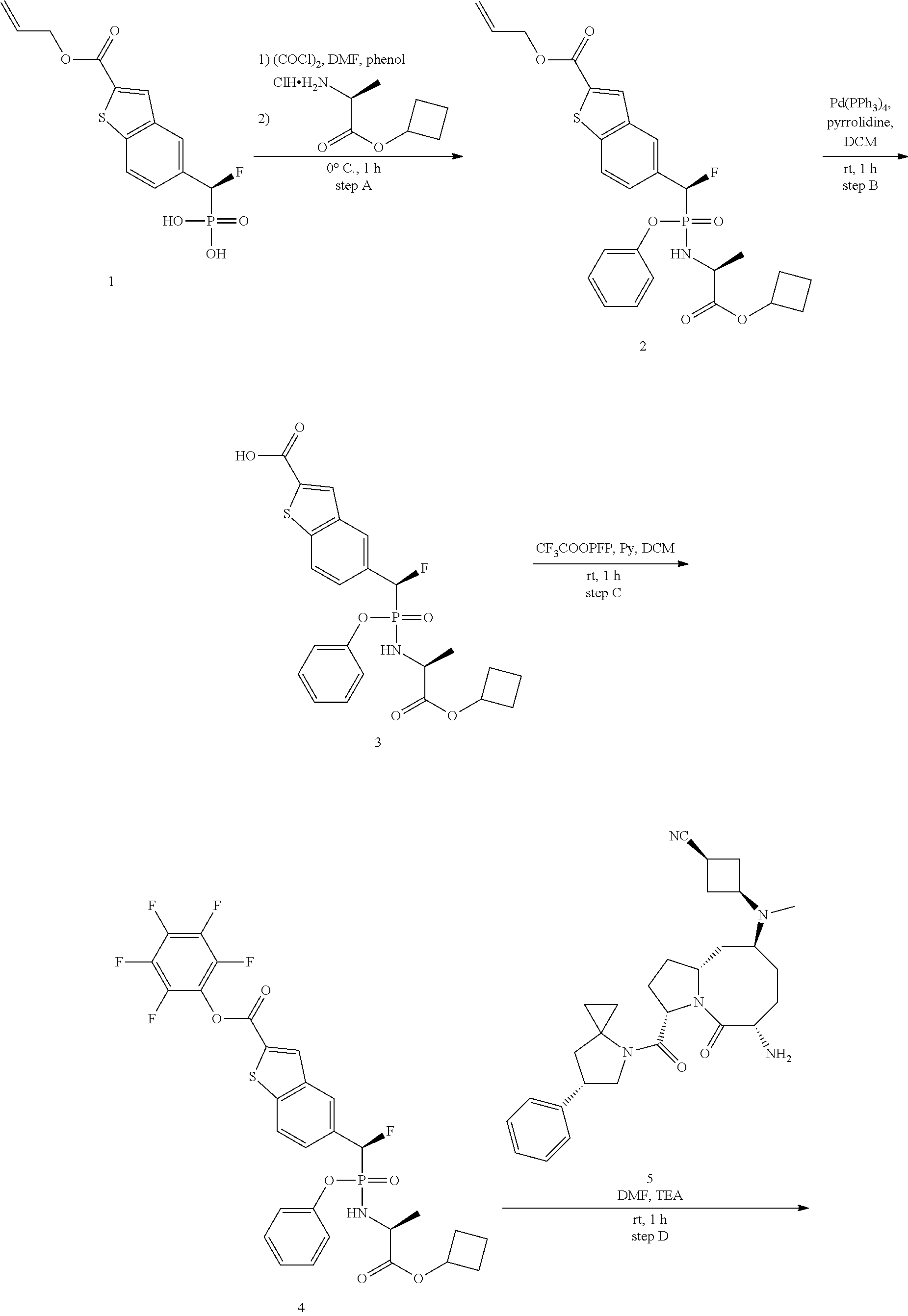

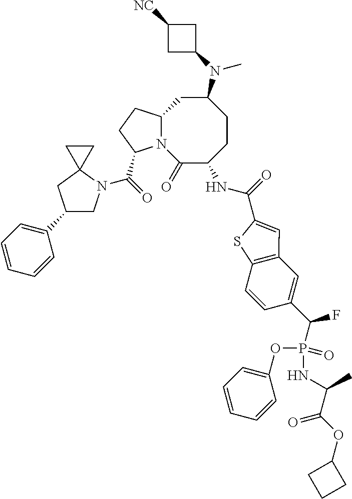 [SMILES: C[C@H](NP(=O)(Oc1ccccc1)[C@H](F)c1ccc2sc(C(=O)N[C@H]3CC[C@H](N(C)[C@H]4C[C@@H](C#N)C4)C[C@H]4CC[C@@H](C(=O)N5C[C@H](c6ccccc6)CC56CC6)N4C3=O)cc2c1)C(=O)OC1CCC1]

### Step A: allyl 5-((1S)—((((S)-1-cyclobutoxy-1-oxopropan-2-yl)amino)(phenoxy)phosphoryl)fluoromethyl)benzo[b]thiophene-2-carboxylate

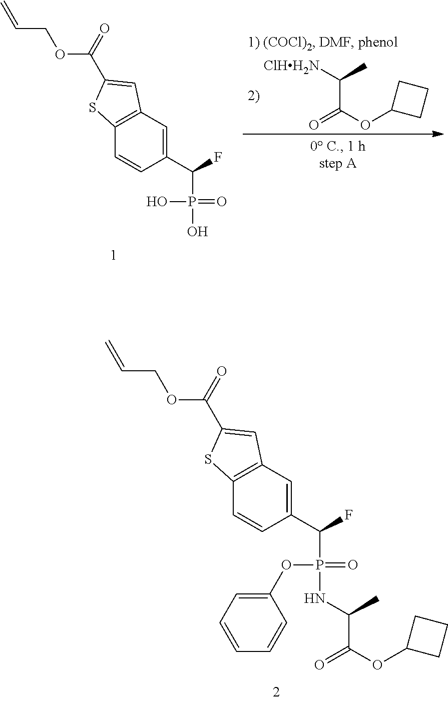

To a solution of (S)-((2-((allyloxy)carbonyl)benzo[b]thiophen-5-yl)fluoromethyl)phosphonic acid (1, 1.3 g, 3.94 mmol, 1 eq.) in DCM (20 mL) was added DMF (1 drop) and oxalyl chloride (2.5 g, 19.7 mmol, 5 eq). The resulting mixture was stirred for 1 h at room temperature, then DCM was removed by reduced pressure. The residue was dissolved in DCM (20 mL) and cooled to 0° C., phenol (285 mg, 3.94 mmol, 1 eq) was added to the solution and TEA (2 g, 19.7 mmol, 5 eq.) was added dropwise then the mixture was stirred for 1 h at 0° C. Then cyclobutyl L-alaninate hydrochloride (4, 1.41 g, 7.88 mmol, 2 eq.) was added to the solution, then H₂O (50 mL) was added to the solution, then extracted with DCM (50 mL×2). The organic layers were combined and washed with brine, then dried over Na₂SO₄, concentrated to dryness under reduced pressure The crude product was purified by silica gel column chromatography to afford allyl 5-((1S)—((((S)-1-cyclobutoxy-1-oxopropan-2-yl)amino)(phenoxy)phosphoryl)fluoromethyl)benzo[b]thiophene-2-carboxylate (2, 800 mg, 1.51 mmol, 38% yield) as a colorless oil. LCMS (ESI): m/z=532 [M+H]⁺.

### Step B: 5-((1S)—((((S)-1-cyclobutoxy-1-oxopropan-2-yl)amino)(phenoxy)phosphoryl)fluoromethyl)benzo[b]thiophene-2-carboxylic acid

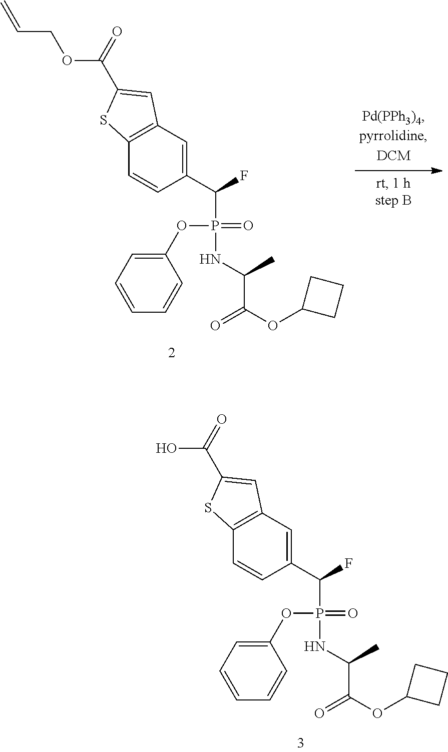 [SMILES: C=CCOC(=O)c1cc2cc([C@@H](F)P(=O)(N[C@@H](C)C(=O)OC3CCC3)Oc3ccccc3)ccc2s1 ; C[C@H](NP(=O)(Oc1ccccc1)[C@H](F)c1ccc2sc(C(=O)O)cc2c1)C(=O)OC1CCC1]

To a solution of allyl 5-((1S)—((((S)-1-cyclobutoxy-1-oxopropan-2-yl)amino)(phenoxy)phosphoryl)fluoromethyl)benzo[b]thiophene-2-carboxylate (2, 800 mg, 1.51 mmol, 1 eq.) in DCM (10 mL) was added Pd(PPh₃)₄ (173 mg, 0.15 mmol, 0.1 eq.) and pyrrolidine (107 mg, 1.51 mmol, 1 eq.). The resulting mixture was stirred for 1 h at room temperature under the atmosphere of N₂, then adjusted pH=3 with 1M HCl (aq.) extracted with DCM (20 mL×2). The organic layers were combined and washed with brine (20 mL) dried over Na₂SO₄, The resulting mixture was concentrated to dryness under reduced pressure to afford crude product 5-((1S)—((((S)-1-cyclobutoxy-1-oxopropan-2-yl)amino)(phenoxy)phosphoryl)fluoromethyl)benzo[b]thiophene-2-carboxylic acid (3, 650 mg, 1.32 mmol, 88% yield) as a yellow solid. LCMS (ESI): m/z=492 [M+H]⁺.

### Step C: perfluorophenyl 5-((1S)—((((S)-1-cyclobutoxy-1-oxopropan-2-yl)amino)(phenoxy)phosphoryl)fluoromethyl)benzo[b]thiophene-2-carboxylate

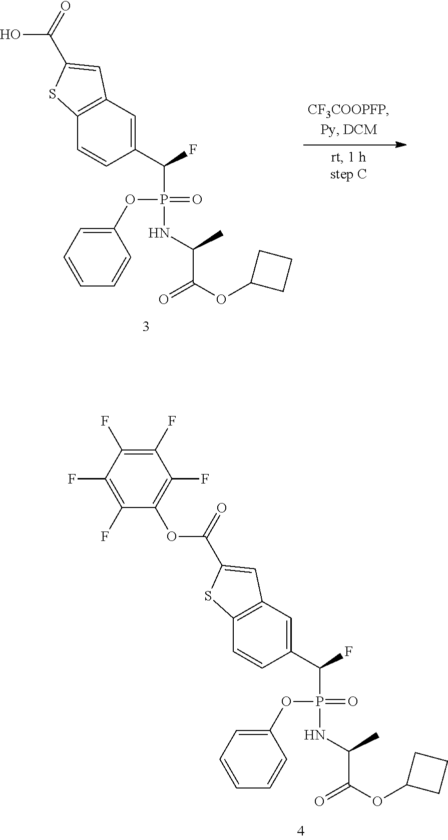 [SMILES: C[C@H](NP(=O)(Oc1ccccc1)[C@H](F)c1ccc2sc(C(=O)O)cc2c1)C(=O)OC1CCC1 ; C[C@H](NP(=O)(Oc1ccccc1)[C@H](F)c1ccc2sc(C(=O)Oc3c(F)c(F)c(F)c(F)c3F)cc2c1)C(=O)OC1CCC1]

To a solution of 5-((1S)—((((S)-1-cyclobutoxy-1-oxopropan-2-yl)amino)(phenoxy)phosphoryl)fluoromethyl)benzo[b]thiophene-2-carboxylic acid (3, 650 mg, 1.32 mmol, 1 eq.) in DCM (10 mL) was added pyridine (106 mg, 1.32 mmol, 3 eq.), perfluorophenyl 2,2,2-trifluoroacetate (741 mg, 2.64 mmol, 2 eq.). The resulting mixture was stirred for 1 h at room temperature, then the reaction was quenched by adding H₂O (20 mL), then extracted with DCM (20 mL×2), the organic layers were combined and washed with brine (20 mL), dried over Na₂SO₄, then concentrated to dryness under reduced pressure. The crude product was purified by silica gel column chromatography to afford perfluorophenyl 5-((1S)—((((S)-1-cyclobutoxy-1-oxopropan-2-yl)amino)(phenoxy)phosphoryl)fluoromethyl)benzo[b]thiophene-2-carboxylate (4, 620 mg, 0.942 mmol, 71% yield) as a white solid. LCMS (ESI): m/z=658 [M+H]⁺.

### Step D: cyclobutyl (((S)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate

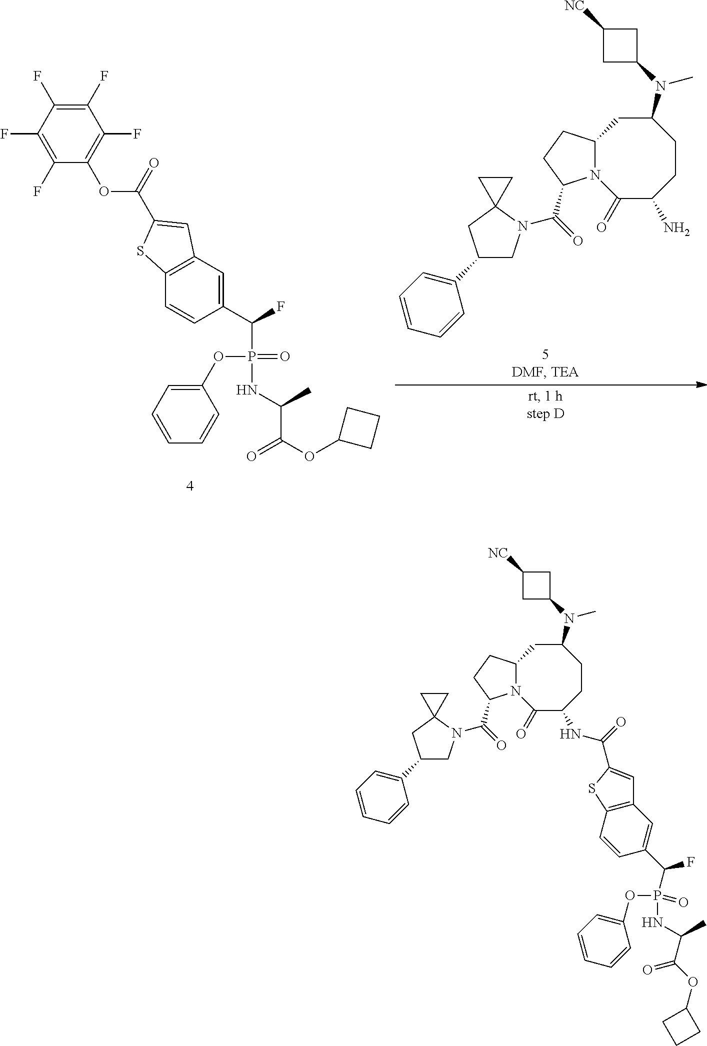 [SMILES: C[C@H](NP(=O)(Oc1ccccc1)[C@H](F)c1ccc2sc(C(=O)Oc3c(F)c(F)c(F)c(F)c3F)cc2c1)C(=O)OC1CCC1 ; CN([C@H]1CC[C@H](N)C(=O)N2[C@H](CC[C@H]2C(=O)N2C[C@H](c3ccccc3)CC23CC3)C1)[C@H]1C[C@@H](C#N)C1 ; C[C@H](NP(=O)(Oc1ccccc1)[C@H](F)c1ccc2sc(C(=O)N[C@H]3CC[C@H](N(C)[C@H]4C[C@@H](C#N)C4)C[C@H]4CC[C@@H](C(=O)N5C[C@H](c6ccccc6)CC56CC6)N4C3=O)cc2c1)C(=O)OC1CCC1]

To a solution of perfluorophenyl 5-((1S)—((((S)-1-cyclobutoxy-1-oxopropan-2-yl)amino)(phenoxy)phosphoryl)fluoromethyl)benzo[b]thiophene-2-carboxylate (4, 20 mg, 0.030 mml, 1 eq.) in DMF (3 mL) was added (1R,3s)-3-(((3S,6S,9S,10aR)-6-amino-5-oxo-3-((S)-6-phenyl-4-azaspiro[2.4]heptane-4-carbonyl)decahydropyrrolo[1,2-a]azocin-9-yl)(methyl)amino)cyclobutane-1-carbonitrile (5, 15 mg, 0.030 mmol, 1 eq.), TEA (6.1 mg, 0.061 mmol, 2 eq). Procedure for 5 is described in syntheses of Compound A37 and Compound C110. The resulting mixture was stirred for 1 h at room temperature, then the crude product was purified by Prep-HPLC to afford cyclobutyl (((S)-(2-(((3S,6S,9S,10aR)-9-(((1s,3R)-3-cyanocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C112, 13 mg, 0.014 mmol, 69% yield) as a white solid. LCMS (ESI): m/z=963 [M+H]⁺. ¹H NMR (400 MHz, DMSO-d₆) δ (ppm) 8.80 (t, J=7.1 Hz, 1H), 8.30-8.24 (m, 1H), 8.10-8.03 (m, 2H), 7.63-7.57 (m, 1H), 7.40-7.27 (m, 6H), 7.24-7.10 (m, 4H), 6.34-6.00 (m, 2H), 4.87-4.65 (m, 2H), 4.47 (t, J=8.4 Hz, 1H), 4.33-4.25 (m, 1H), 4.12 (t, J=8.5 Hz, 1H), 3.88-3.48 (m, 3H), 3.07-2.89 (m, 3H), 2.38-2.29 (m, 2H), 2.26-2.11 (m, 5H), 2.05-1.47 (m, 20H), 1.20-0.87 (m, 3H), 0.55-0.42 (m, 2H). ¹⁹F NMR (377 MHz, DMSO-d₆) δ (ppm) −194.13 (d, J=82.6 Hz), −195.92 (d, J=87.3 Hz). ³¹P NMR (162 MHz, DMSO-d₆) δ (ppm) 17.29 (d, J=86.7 Hz), 16.94 (d, J=82.5 Hz).

### Synthesis of ((S)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A38) and propyl (((S)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(2,2,2-trifluoroethoxy)phosphoryl)-L-alaninate (Compound C87)

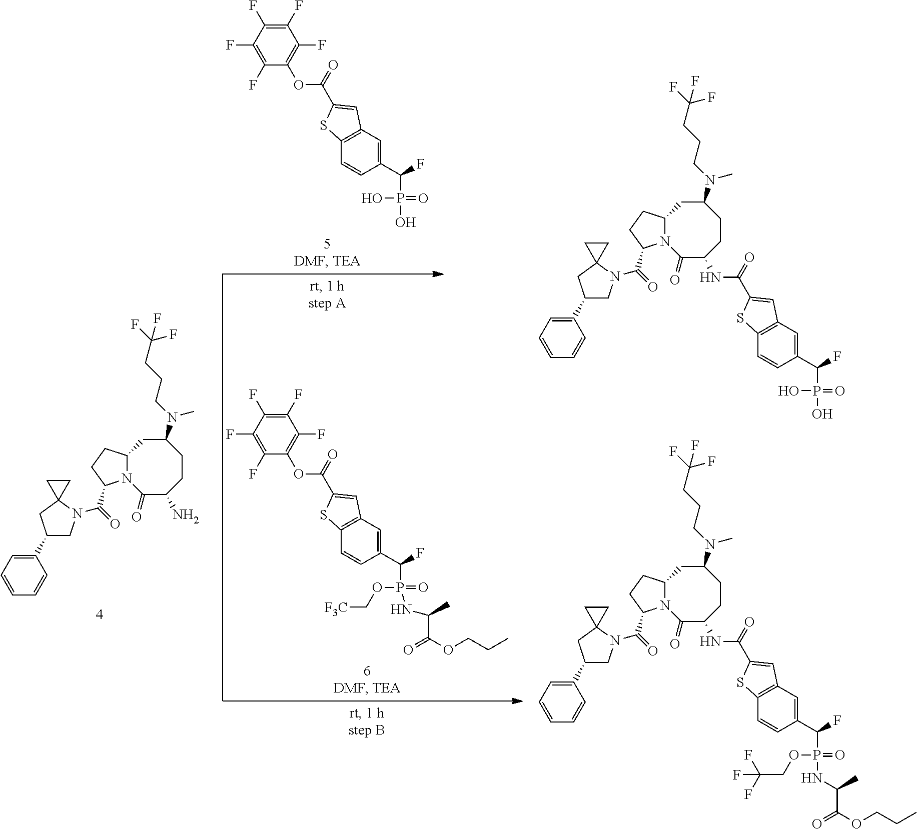

### Step A: ((S)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

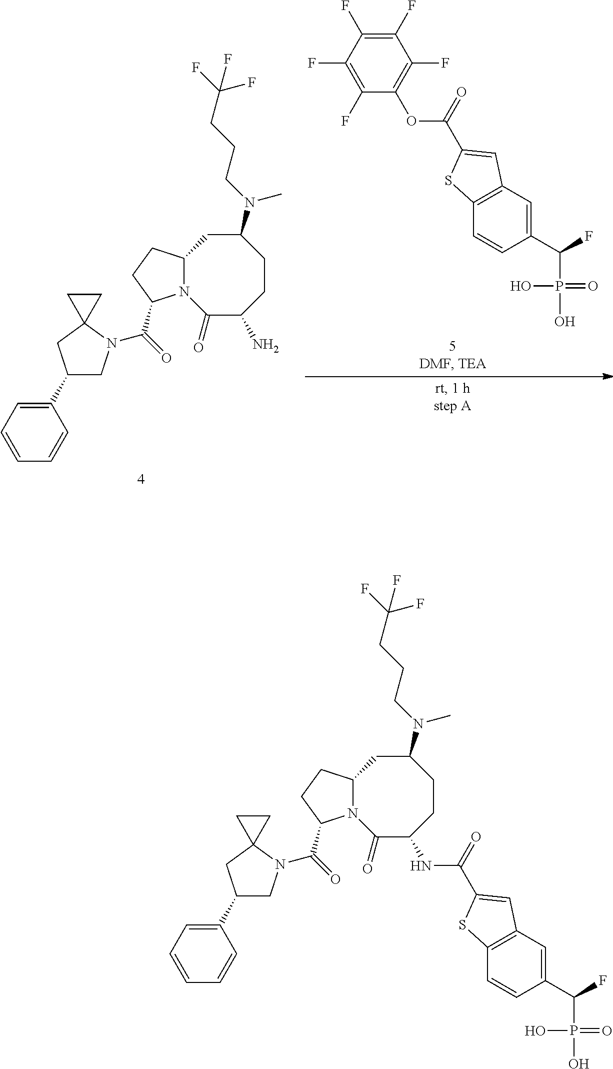 [SMILES: CN(CCCC(F)(F)F)[C@H]1CC[C@H](N)C(=O)N2[C@H](CC[C@H]2C(=O)N2C[C@H](c3ccccc3)CC23CC3)C1 ; O=C(Oc1c(F)c(F)c(F)c(F)c1F)c1cc2cc([C@@H](F)P(=O)(O)O)ccc2s1 ; CN(CCCC(F)(F)F)[C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1]

Procedure for 4 is described in the synthesis of Compound A3 and Compound C135. To a solution of (3S,6S,9S,10aR)-6-amino-9-(methyl(4,4,4-trifluorobutyl)amino)-3-((S)-6-phenyl-4-azaspiro[2.4]heptane-4-carbonyl)octahydropyrrolo[1,2-a]azocin-5(1H)-one (4, 20 mg, TFA salt) in DMF (2 mL) was added TEA (6.5 mg, 0.065 mmol, 2 eq.), (S)-(fluoro(2-((perfluorophenoxy)carbonyl)benzo[b]thiophen-5-yl)methyl)phosphonic acid (5, 14.67 mg, 0.032 mmol, 1 eq). The resulting mixture was stirred for 1 h at room temperature, then the crude product was purified by Prep-HPLC to afford ((S)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A38, 17 mg, 0.021 mmol) as a white solid. LCMS (ESI): m/z=793 [M+H]⁺. 1H NMR (400 MHz, DMSO-d₆) δ (ppm) 9.72-9.32 (m, 1H), 8.91-8.69 (m, 1H), 8.25 (s, 1H), 8.08-7.94 (m, 2H), 7.53 (d, J=8.5 Hz, 1H), 7.38-7.28 (m, 4H), 7.25-7.20 (m, 1H), 5.81 (dd, J=44.3, 7.9 Hz, 1H), 4.81 (s, 1H), 4.60-4.50 (m, 2H), 4.17-4.09 (m, 1H), 3.90-3.69 (m, 2H), 3.63-3.51 (m, 1H), 3.23-3.00 (m, 2H), 2.79-2.68 (m, 3H), 2.41-1.75 (m, 17H), 1.58-1.49 (m, 1H), 0.57-0.42 (m, 2H). ¹⁹F NMR (376 MHz, DMSO d₆) δ (ppm) −64.73 (d, J=58.3 Hz), −194.65 (dd, J=82.9, 16.4 Hz). ³¹P NMR (162 MHz, DMSO-d₆) δ (ppm) 10.87 (d, J=80.8 Hz).

### Step B: propyl (((S)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(2,2,2-trifluoroethoxy)phosphoryl)-L-alaninate

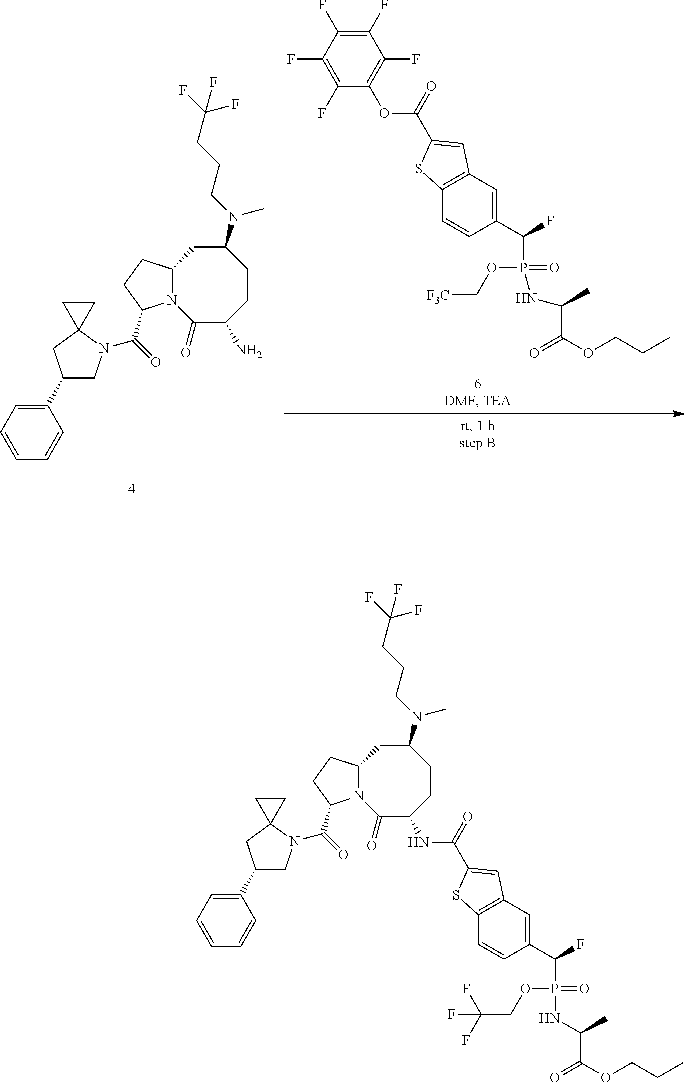 [SMILES: CN(CCCC(F)(F)F)[C@H]1CC[C@H](N)C(=O)N2[C@H](CC[C@H]2C(=O)N2C[C@H](c3ccccc3)CC23CC3)C1 ; CCCOC(=O)[C@H](C)NP(=O)(OCC(F)(F)F)[C@H](F)c1ccc2sc(C(=O)Oc3c(F)c(F)c(F)c(F)c3F)cc2c1 ; CCCOC(=O)[C@H](C)NP(=O)(OCC(F)(F)F)[C@H](F)c1ccc2sc(C(=O)N[C@H]3CC[C@H](N(C)CCCC(F)(F)F)C[C@H]4CC[C@@H](C(=O)N5C[C@H](c6ccccc6)CC56CC6)N4C3=O)cc2c1]

Procedure for 6 is described in the synthesis of Compound C64. To a solution of perfluorophenyl 5-((1S)-fluoro((((S)-1-oxo-1-propoxypropan-2-yl)amino)(2,2,2-trifluoroethoxy)phosphoryl)methyl)benzo[b]thiophene-2-carboxylate (6, 15 mg, 0.023 mml, 1 eq.) in DMF (3 mL) was added (3S,6S,9S,10aR)-6-amino-9-(methyl(4,4,4-trifluorobutyl)amino)-3-((S)-6-phenyl-4-azaspiro[2.4]heptane-4-carbonyl)octahydropyrrolo[1,2-a]azocin-5(1H)-one (4, 14 mg, 0.023 mmol, 1 eq.), TEA (14 mg, 0.138 mmol, 6 eq). The resulting mixture was stirred for 1 h at room temperature, then the crude product was purified by Prep-HPLC to afford propyl (((S)-fluoro(2-(((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(2,2,2-trifluoroethoxy)phosphoryl)-L-alaninate (Compound C87, 7 mg, 0.007 mmol) as a white solid. LCMS (ESI): m/z=988 [M+H]⁺. ¹H NMR (400 MHz, DMSO-d₆) δ (ppm) 8.84-8.72 (m, 1H), 8.26 (s, 1H), 8.11-8.02 (m, 1H), 7.98 (d, J=9.6 Hz, 1H), 7.58-7.46 (m, 1H), 7.39-7.26 (m, 4H), 7.24-7.19 (m, 1H), 6.27-5.92 (m, 2H), 4.84-4.73 (m, 1H), 4.65-4.42 (m, 3H), 4.36-4.26 (m, 1H), 4.16-4.06 (m, 1H), 4.05-3.93 (m, 2H), 3.84-3.63 (m, 2H), 3.58-3.49 (m, 1H), 3.01-2.89 (m, 1H), 2.47-2.43 (m, 1H), 2.35-2.20 (m, 4H), 2.16-2.08 (m, 4H), 2.04-1.94 (m, 3H), 1.90-1.48 (m, 13H), 1.31-1.02 (m, 3H), 0.89-0.80 (m, 3H), 0.54-0.39 (m, 2H). ¹⁹F NMR (376 MHz, DMSO-d₆) δ (ppm) −64.56 (s), −73.67 (d, J=22.0 Hz), −195.22 (d, J=83.8 Hz), −197.00 (d, J=86.1 Hz). ³¹P NMR (162 MHz, DMSO-d₆) δ (ppm) 21.32 (d, J=83.8 Hz), 20.98 (d, J=86.2 Hz).

### Synthesis of propyl (((R)-fluoro(2-(((3S,6S,9S,10aR)-9-(((1s,3R)-3-fluorocyclobutyl)(methyl)amino)-5-oxo-3-(4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C85) and ((R)-fluoro(2-(((3S,6S,9S,10aR)-9-(((1s,3R)-3-fluorocyclobutyl)(methyl)amino)-5-oxo-3-(4-azaspiro[2.4]heptane-4-carbonyl)decahydropyrrolo[1,2-a]azocin-6-yl)carbamoyl)benzo[b]thiophen-5-yl)methyl)phosphonic acid (Compound A39)

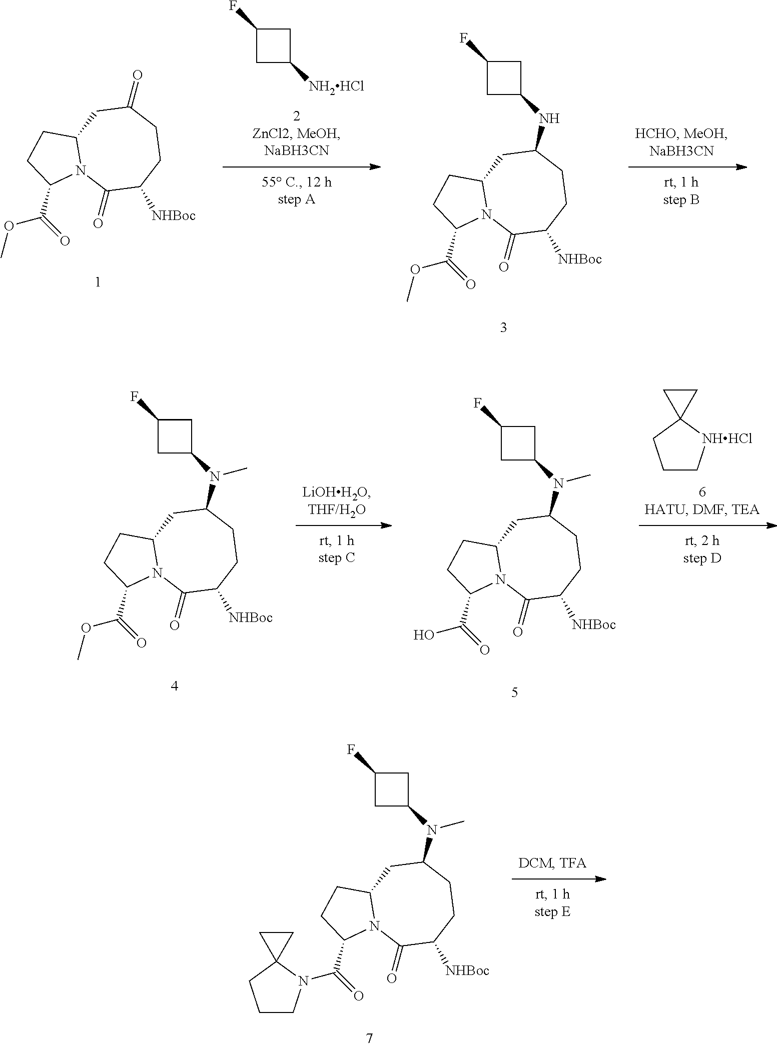

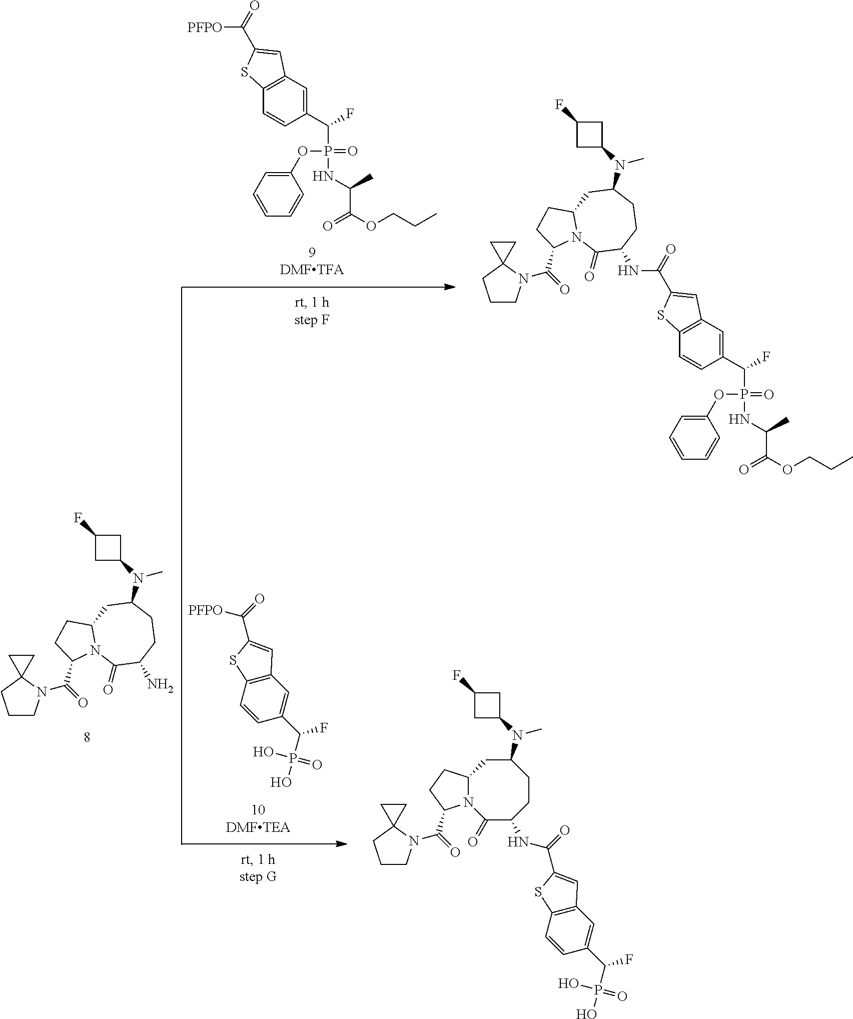

### Step A: methyl (3S,6S,9S,10aR)-6-((tert-butoxycarbonyl)amino)-9-(((1s,3R)-3-fluorocyclobutyl)amino)-5-oxodecahydropyrrolo[1,2-a]azocine-3-carboxylate

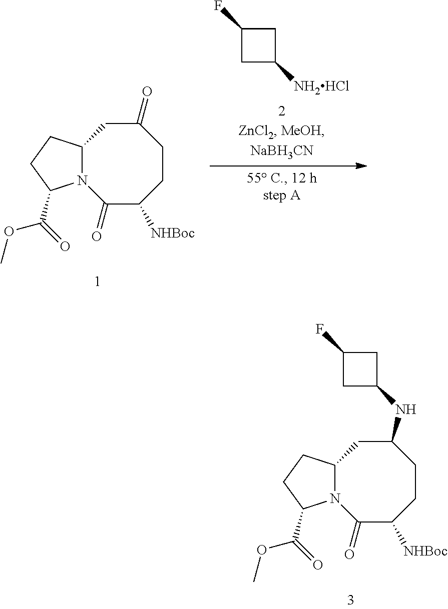

To a solution of methyl (3S,6S,10aR)-6-((tert-butoxycarbonyl)amino)-5,9-dioxodecahydropyrrolo[1,2-a]azocine-3-carboxylate (1, 1 g, 2.82 mmol, 1.0 eq.) and (1s,3s)-3-fluorocyclobutan-1-amine hydrochloride (2, 635 mg, 5.08 mmol, 1.8 eq.) in MeOH (10 mL) was added ZnCl₂ (1.89 g, 14.1 mmol, 5.0 eq.). The resulting mixture was stirred at 55° C. for 1 hr under N₂, then NaBH₃CN (1.78 g, 28.2 mmol, 10.0 eq.) was added portion wise every 30 mins (2 eq. (356 mg each time) added 5 times) and then the resulting mixture was stirred at 55° C. for 12 h under N₂. The reaction mixture was cooled to room temperature then used to next step directly without further purification. LCMS (ESI): m/z=428 [M+H]⁺.

### Step B: methyl (3S,6S,9S,10aR)-6-((tert-butoxycarbonyl)amino)-9-(((1s,3R)-3-fluorocyclobutyl)(methyl)amino)-5-oxodecahydropyrrolo[1,2-a]azocine-3-carboxylate

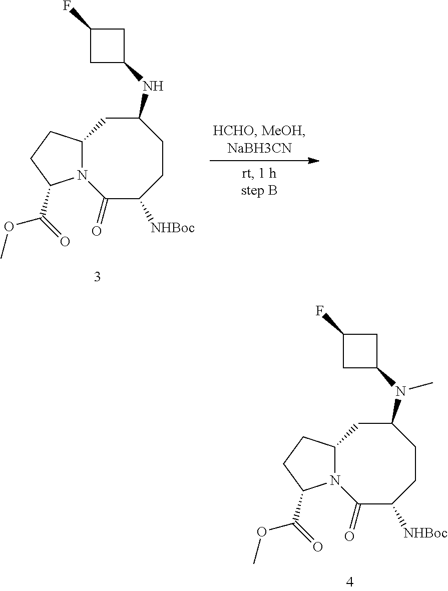

To a mixture of methyl (3S,6S,9S,10aR)-6-((tert-butoxycarbonyl)amino)-9-(((1s,3R)-3-fluorocyclobutyl)amino)-5-oxodecahydropyrrolo[1,2-a]azocine-3-carboxylate (3, step A above, crude.) were added HCHO (2 mL, 30 wt % in water) and NaBH₃CN (355 mg, 5.64 mmol, 2.0 eq.). The resulting mixture was stirred at 55° C. for 30 min under N₂ then the reaction was quenched by NH₄Cl (sat. aq), diluted with H₂O (50 mL), then extracted with EtOAc (30 mL×3). The organic layers were combined and washed with brine (10 mL), dried over Na₂SO₄, then concentrated to dryness under reduced pressure. The crude product was purified by reverse phase chromatography using a 40 g C18 cartridge eluting with a gradient of 5-80% MeCN in water (with 0.1% NH₃H₂O) to afford methyl (3S,6S,9S,10aR)-6-((tert-butoxycarbonyl)amino)-9-(((1s,3R)-3-fluorocyclobutyl)(methyl)amino)-5-oxodecahydropyrrolo[1,2-a]azocine-3-carboxylate (4, 0.6 g, 1.36 mmol, 48% over 2 steps). LCMS (ESI): m/z=442 [M+H]⁺.

### Step C: (3S,6S,9S,10aR)-6-((tert-butoxycarbonyl)amino)-9-(((1s,3R)-3-fluorocyclobutyl)(methyl)amino)-5-oxodecahydropyrrolo[1,2-a]azocine-3-carboxylic acid

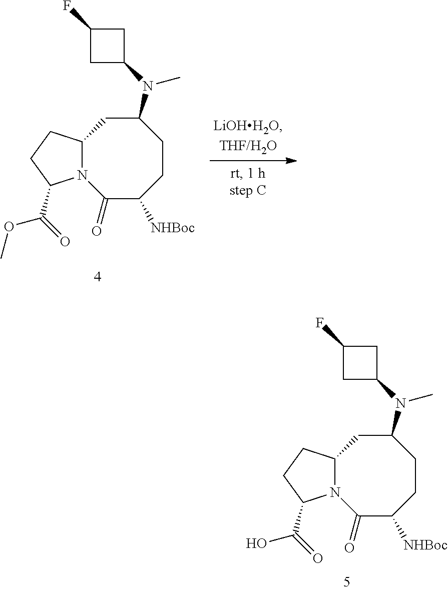

To a solution of methyl (3S,6S,9S,10aR)-6-((tert-butoxycarbonyl)amino)-9-(((1s,3R)-3-fluorocyclobutyl)(methyl)amino)-5-oxodecahydropyrrolo[1,2-a]azocine-3-carboxylate (4, 600 mg, 1.36 mmol, 1 eq) in a 3:1 mixture of THF/H₂O (12 mL) was added lithium hydroxide monohydrate (171.1 mg, 4.08 mmol, 3 eq.), and the resulting mixture was stirred at room temperature for 1 h, then the reaction mixture was adjusted pH=3 with 1M HCl (aq.), then extracted with EtOAc (15 mL*3). The combined organic layers were washed with brine (10 mL), dried over anhydrous Na₂SO₄, filtered and concentrated under reduced pressure to give (3S,6S,9S,10aR)-6-((tert-butoxycarbonyl)amino)-9-(((1s,3R)-3-fluorocyclobutyl)(methyl)amino)-5-oxodecahydropyrrolo[1,2-a]azocine-3-carboxylic acid (5, 500 mg, 1.17 mmol, 86%) as a white solid. LCMS (ESI): m/z=428 [M+H]⁺.

### Step D: tert-butyl ((3S,6S,9S,10aR)-9-(((1s,3R)-3-fluorocyclobutyl)(methyl)amino)-5-oxo-3-(4-azaspiro[2.4]heptane-4-carbonyl)decahydropyrrolo[1,2-a]azocin-6-yl)carbamate

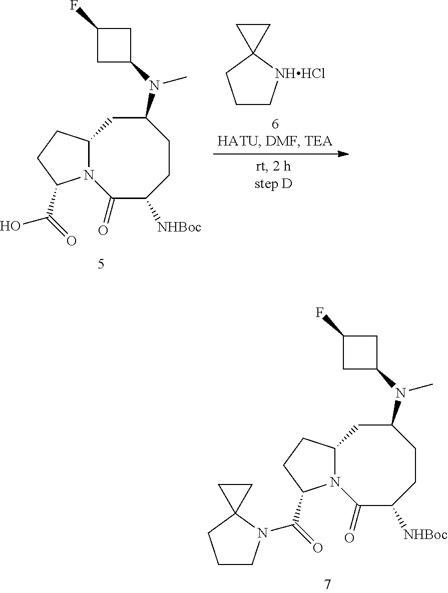

To a solution of (3S,6S,9S,10aR)-6-((tert-butoxycarbonyl)amino)-9-(((1s,3R)-3-fluorocyclobutyl)(methyl)amino)-5-oxodecahydropyrrolo[1,2-a]azocine-3-carboxylic acid (5, 100 mg, 0.234 mmol, 1 eq) in DMF (3 mL) were added TEA (70.9 mg, 0.702 mmol, 3 eq.), HATU (97.8 mg, 0.257 mmol, 1.1 eq.) and 4-azaspiro[2.4]heptane hydrochloride (6, 34.4 mg, 0.257 mmol, 1.1 eq.), and the resulting mixture was stirred at room temperature for 2 h. LCMS indicated the complete consumption of the starting material. The reaction mixture was purified by reverse phase chromatography using a C18 column eluting with 20-70% CH₃CN in water (0.1% TFA) to give tert-butyl ((3S,6S,9S,10aR)-9-(((1s,3R)-3-fluorocyclobutyl)(methyl)amino)-5-oxo-3-(4-azaspiro[2.4]heptane-4-carbonyl)decahydropyrrolo[1,2-a]azocin-6-yl)carbamate (7, 105 mg, 0.207 mmol, 88.5%) as a white solid. LCMS (ESI): m/z=507 [M+H]⁺.

### Step E: (3S,6S,9S,10aR)-6-amino-9-(((1s,3R)-3-fluorocyclobutyl)(methyl)amino)-3-(4-azaspiro[2.4]heptane-4-carbonyl)octahydropyrrolo[1,2-a]azocin-5(1H)-one

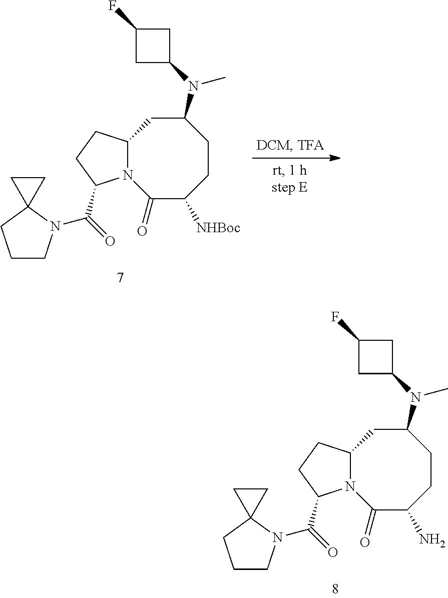 [SMILES: CN([C@H]1CC[C@H](NC(=O)OC(C)(C)C)C(=O)N2[C@H](CC[C@H]2C(=O)N2CCCC23CC3)C1)[C@H]1C[C@@H](F)C1 ; CN([C@H]1CC[C@H](N)C(=O)N2[C@H](CC[C@H]2C(=O)N2CCCC23CC3)C1)[C@H]1C[C@@H](F)C1]

To a solution of tert-butyl ((3S,6S,9S,10aR)-9-(((1s,3R)-3-fluorocyclobutyl)(methyl)amino)-5-oxo-3-(4-azaspiro[2.4]heptane-4-carbonyl)decahydropyrrolo[1,2-a]azocin-6-yl)carbamate (7, 80 mg, 0.158 mmol, 1 eq.) in DCM (2 mL) was added TFA (2 mL), The resulting mixture was stirred for 1 h at room temperature, LCMS show that the reaction was completed. The resulting mixture was concentrated to dryness under reduced pressure to afford crude product (3S,6S,9S,10aR)-6-amino-9-(((1s,3R)-3-fluorocyclobutyl)(methyl)amino)-3-(4-azaspiro[2.4]heptane-4-carbonyl)octahydropyrrolo[1,2-a]azocin-5(1H)-one (8, 80 mg, TFA salt) as a yellow oil which used in the next step without further purification. LCMS (ESI): m/z=407 [M+H]⁺.

### Step F: propyl (((R)-fluoro(2-(((3S,6S,9S,10aR)-9-(((1s,3R)-3-fluorocyclobutyl)(methyl)amino)-5-oxo-3-(4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

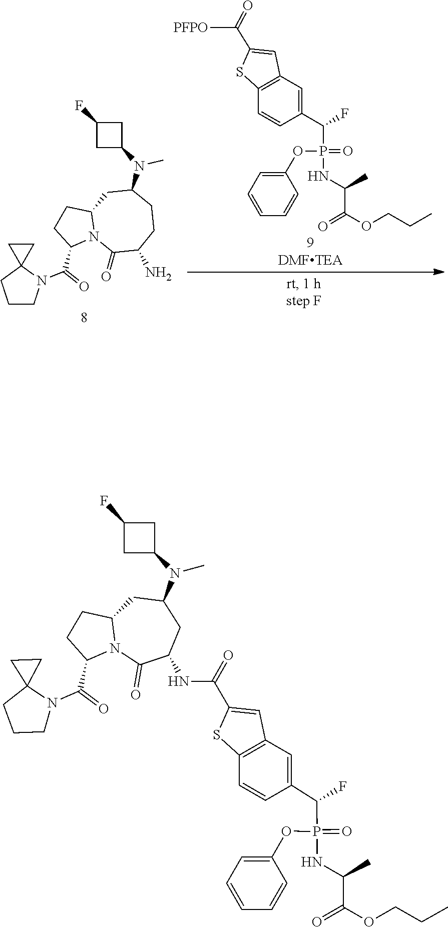

To a solution of (3S,6S,9S,10aR)-6-amino-9-(((1s,3R)-3-fluorocyclobutyl)(methyl)amino)-3-(4-azaspiro[2.4]heptane-4-carbonyl)octahydropyrrolo[1,2-a]azocin-5(1H)-one (8, 40 mg, TFA salt) in DMF (2 mL) was added TEA (23.9 mg, 0.228 mmol, 3 eq.), perfluorophenyl 5-((1R)-fluoro((((S)-1-oxo-1-propoxypropan-2-yl)amino)(phenoxy)phosphoryl)methyl)benzo[b]thiophene-2-carboxylate (9, 50 mg, 0.076 mmol, 1 eq.). The resulting mixture was stirred for 1 h at room temperature, then the crude product was purified by Prep-HPLC to give propyl (((R)-fluoro(2-(((3S,6S,9S,10aR)-9-(((1s,3R)-3-fluorocyclobutyl)(methyl)amino)-5-oxo-3-(4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C85, 9 mg, 0.0103 mmol, 13.5%) as a white solid. LCMS (ESI): m/z=868.5 [M+H]⁺. ¹H NMR (400 MHz, DMSO-d₆) δ (ppm) 8.84-8.71 (m, 1H), 8.27 (s, 1H), 8.10-8.03 (m, 2H), 7.61 (d, J=8.1 Hz, 1H), 7.36 (t, J=7.8 Hz, 2H), 7.21-7.11 (m, 3H), 6.27-6.06 (m, 2H), 4.90-4.65 (m, 2H), 4.45 (t, J=8.5 Hz, 1H), 4.33-4.23 (m, 1H), 3.97-3.73 (m, 4H), 3.64-3.54 (m, 1H), 2.97 (s, 1H), 2.65-2.55 (m, 1H), 2.41-2.24 (m, 2H), 2.13-1.92 (m, 6H), 1.91-1.37 (m, 17H), 1.17-1.09 (m, 3H), 0.86-0.75 (m, 3H), 0.46-0.35 (m, 2H). 1⁹F NMR (377 MHz, DMSO-d₆) δ (ppm) −165.30 (s), −194.33 (d, J=82.9 Hz), −196.33 (d, J=86.1 Hz). ³¹P NMR (162 MHz, DMSO-d₆) δ (ppm) 17.32 (d, J=82.6 Hz), 17.07 (d, J=86.5 Hz).

### Step G: ((R)-fluoro(2-(((3S,6S,9S,10aR)-9-(((1s,3R)-3-fluorocyclobutyl)(methyl)amino)-5-oxo-3-(4-azaspiro[2.4]heptane-4-carbonyl)decahydropyrrolo[1,2-a]azocin-6-yl)carbamoyl)benzo[b]thiophen-5-yl)methyl)phosphonic acid

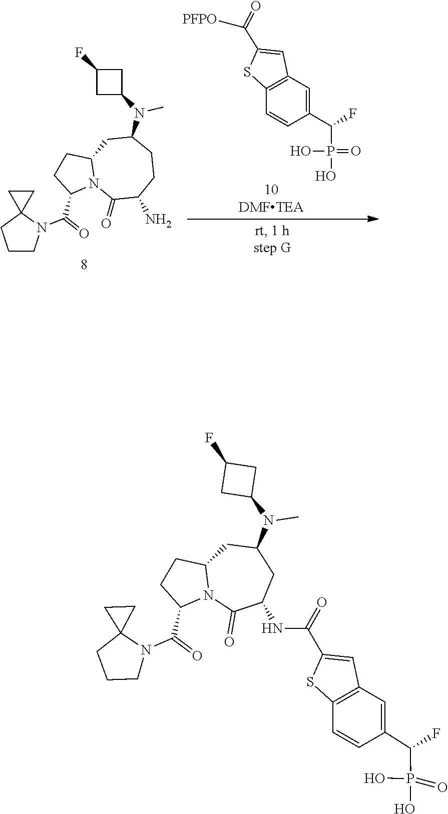

To a solution of (3S,6S,9S,10aR)-6-amino-9-(((1s,3R)-3-fluorocyclobutyl)(methyl)amino)-3-(4-azaspiro[2.4]heptane-4-carbonyl)octahydropyrrolo[1,2-a]azocin-5(1H)-one (8, 40 mg, TFA salt) in DMF (2 mL) was added TEA (23.9 mg, 0.228 mmol, 3 eq.), (R)-(fluoro(2-((perfluorophenoxy)carbonyl)benzo[b]thiophen-5-yl)methyl)phosphonic acid (10, 34 mg, 0.076 mmol, 1 eq). The resulting mixture was stirred for 1 h at room temperature then the crude product was purified by Prep-HPLC to afford ((R)-fluoro(2-(((3S,6S,9S,10aR)-9-(((1s,3R)-3-fluorocyclobutyl)(methyl)amino)-5-oxo-3-(4-azaspiro[2.4]heptane-4-carbonyl)decahydropyrrolo[1,2-a]azocin-6-yl)carbamoyl)benzo[b]thiophen-5-yl)methyl)phosphonic acid (Compound A39, 26.7 mg, 0.039 mmol, 51.3%) as a white solid. LCMS (ESI): m/z=679.4 [M+H]⁺. ¹H NMR (400 MHz, CD₃OD-d₄) δ (ppm) 8.04 (d, J=10.2 Hz, 2H), 7.94 (d, J=8.5 Hz, 1H), 7.60 (d, J=8.3 Hz, 1H), 5.80 (dd, J=44.6, 8.0 Hz, 1H), 4.83-4.74 (m, 2H), 4.72-4.49 (m, 2H), 4.13-3.81 (m, 2H), 3.69-3.43 (m, 2H), 3.00-2.74 (m, 2H), 2.67 (s, 3H), 2.52-2.41 (m, 2H), 2.30-2.13 (m, 3H), 2.06-1.69 (m, 13H), 0.56-0.45 (m, 2H). ¹⁹F NMR (376 MHz, CD₃OD-d₄) δ (ppm) −169.68 (s), −198.76 (d, J=83.9 Hz). ³¹P NMR (162 MHz, CD₃OD-d₄) δ (ppm) 12.46 (d, J=83.5 Hz). (TFA salt).

### Synthesis of propyl (((S)-(2-(((3S,6S,9S,10aR)-9-(ethyl((1r,3S)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C119) and ((S)-(2-(((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A47)

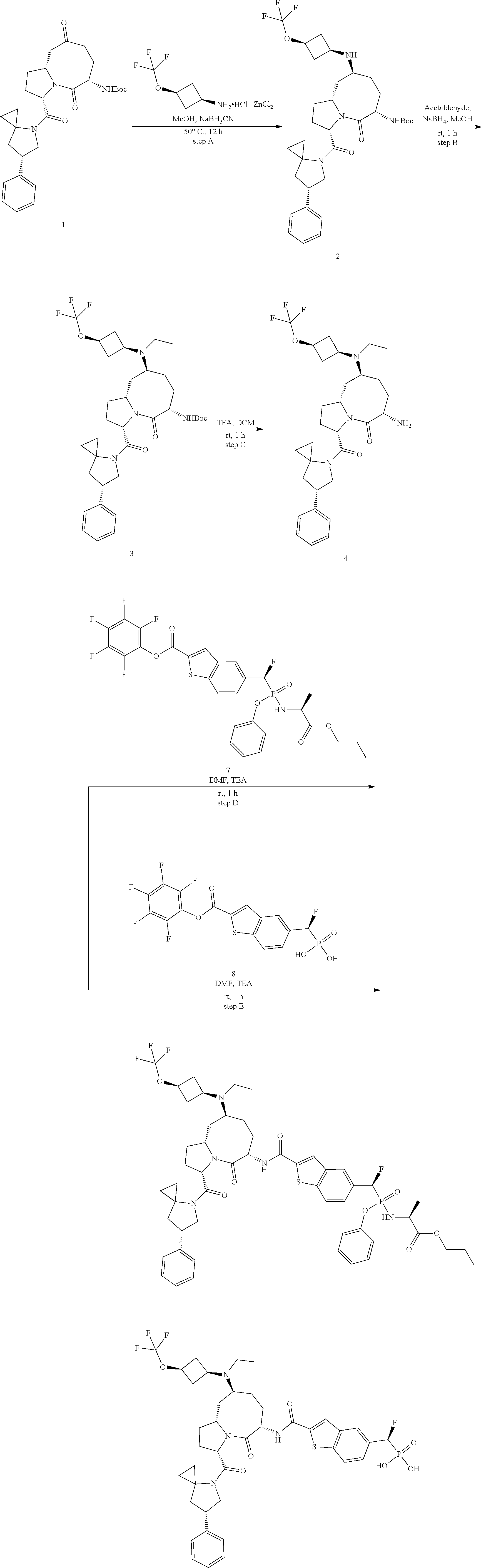

### Step A: tert-butyl ((3S,6S,9S,10aR)-5-oxo-3-((S)-6-phenyl-4-azaspiro[2.4]heptane-4-carbonyl)-9-(((1s,3R)-3-(trifluoromethoxy)cyclobutyl)amino)decahydropyrrolo[1,2-a]azocin-6-yl)carbamate

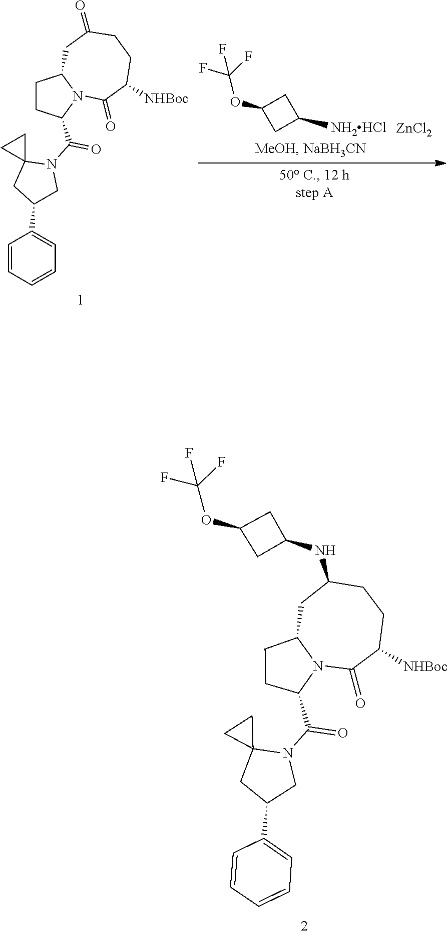

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (1, 1 g, 2.02 mmol, 1 eq.) in MeOH (20 mL) was added (1s,3s)-3-(trifluoromethoxy)cyclobutan-1-amine hydrochloride (579 mg, 3.03 mml, 1.5 eq.), ZnCl₂ (2.75 g, 20.2 mmol, 10 eq.). The resulting mixture was stirred for 1 h at 50° C., then NaBH₃CN (1.27 g, 20.2 mmol, 10 eq.) was added portion wise every 30 mins (2 eq. (254 mg each time) added 5 times). The mixture was stirred for 12 h at 50° C., then cooled to room temperature, and used to the next step directly without further workup. LCMS (ESI): m/z=635 [M+H]⁺.

### Step B: tert-butyl ((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamate

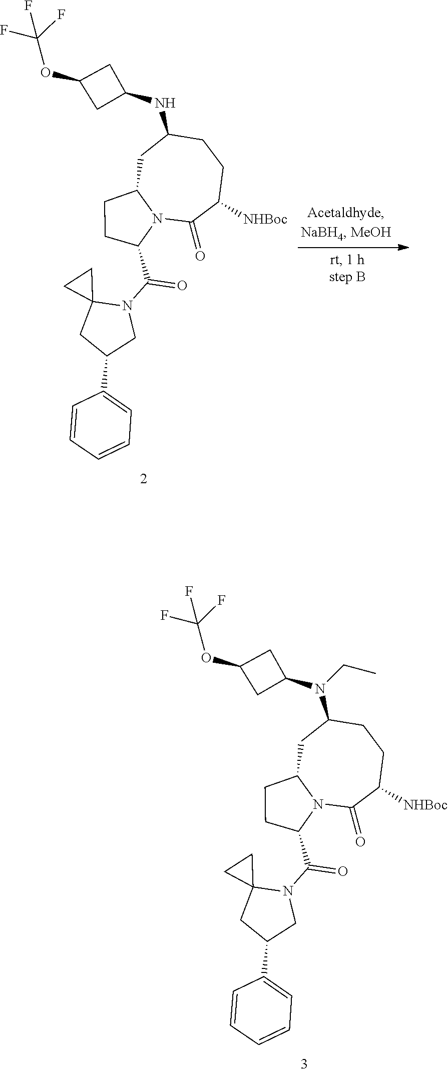 [SMILES: CC(C)(C)OC(=O)N[C@H]1CC[C@H](N[C@H]2C[C@@H](OC(F)(F)F)C2)C[C@H]2CC[C@@H](C(=O)N3C[C@H](c4ccccc4)CC34CC4)N2C1=O ; CCN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](OC(F)(F)F)C1]

To the above mixture (step A) was added acetaldehyde (1 mL) at room temperature, the mixture was stirred for 10 min, then NaBH₄ (767 mg, 20.2 mmol, 10 eq.) was added to the mixture and stirred for another 10 min. The reaction was quenched by NH₄Cl (sat. aq), diluted with H₂O (100 mL), then extracted with EtOAc (50 mL×3). The organic layers were combined and washed with brine (30 mL), dried over Na₂SO₄, then concentrated under reduced pressure. The crude product was purified by C18 column (ACN:H₂O (0.1% ammonium hydroxide)=5%-70%) to afford tert-butyl ((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamate (3, 470 mg, 0.709 mmol, 35.1% yield, two steps) as a white solid. LCMS (ESI): m/z=663 [M+H]⁺.

### Step C: (3S,6S,9S,10aR)-6-amino-9-(ethyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one

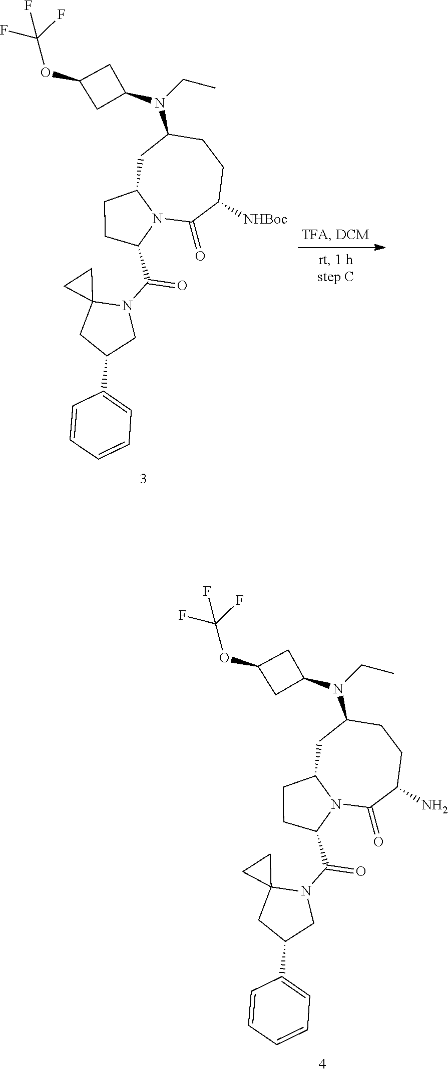 [SMILES: CCN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](OC(F)(F)F)C1 ; CCN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1]

To a solution of tert-butyl ((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamate (3, 120 mg, 0.181 mmol) in DCM (2 mL) was added TFA (2 mL) and the resulting mixture was stirred for 1 h at room temperature, then the resulting mixture was concentrated to dryness under reduced pressure to afford crude product (3S,6S,9S,10aR)-6-amino-9-(ethyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one (4, 30 mg, TFA salt) as a yellow oil which used in the next step without further purification. LCMS (ESI): m/z=563 [M+H]⁺.

### Step D: propyl (((S)-(2-(((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate

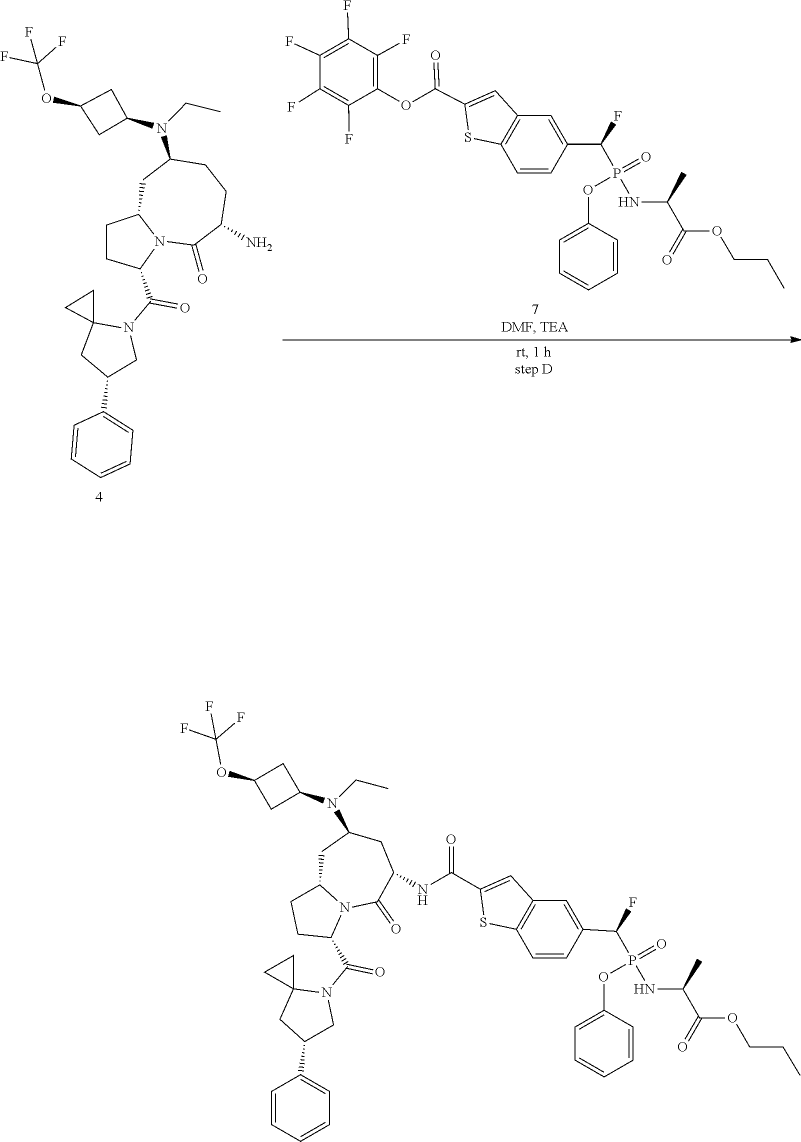 [SMILES: CCN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1 ; CCCOC(=O)[C@H](C)NP(=O)(Oc1ccccc1)[C@H](F)c1ccc2sc(C(=O)Oc3c(F)c(F)c(F)c(F)c3F)cc2c1 ; CCCOC(=O)[C@H](C)NP(=O)(Oc1ccccc1)[C@H](F)c1ccc2sc(C(=O)N[C@H]3C[C@H](N(CC)[C@H]4C[C@@H](OC(F)(F)F)C4)C[C@H]4CC[C@@H](C(=O)N5C[C@H](c6ccccc6)CC56CC6)N4C3=O)cc2c1]

To a solution of (3S,6S,9S,10aR)-6-amino-9-(ethyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one (4, 30 mg, TFA salt) in DMF (2 mL) was added TEA (13.7 mg, 0.135 mmol, 3 eq.), perfluorophenyl 5-((1S)-fluoro((((S)-1-oxo-1-propoxypropan-2-yl)amino)(phenoxy)phosphoryl)methyl)benzo[b]thiophene-2-carboxylate (7, 29 mg, 0.045 mmol, 1 eq). The resulting mixture was stirred for 1 h at room temperature, then the crude product was purified by Prep-HPLC to afford propyl (((S)-(2-(((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C119, 26 mg, 0.025 mmol, 55.5%) as a white solid. LCMS (ESI): m/z=1024.8 [M+H]⁺. 1H NMR (400 MHz, DMSO-d₆) δ (ppm) 8.89-8.68 (m, 1H), 8.27 (d, J=3.8 Hz, 1H), 8.13-8.00 (m, 2H), 7.60 (t, J=7.5 Hz, 1H), 7.40-7.26 (m, 6H), 7.24-7.08 (m, 4H), 6.34-6.04 (m, 2H), 4.77-4.62 (m, 1H), 4.57-4.41 (m, 2H), 4.34-4.22 (m, 1H), 4.12 (t, J=8.4 Hz, 1H), 3.96-3.66 (m, 4H), 3.60-3.47 (m, 1H), 3.11-2.98 (m, 1H), 2.92-2.79 (m, 1H), 2.60-2.54 (m, 1H), 2.47-2.40 (m, 1H), 2.34-1.41 (m, 21H), 1.17 (d, J=7.2 Hz, 1H), 1.01-0.92 (m, 4H), 0.81 (t, J=7.4 Hz, 3H), 0.53-0.33 (m, 2H). ¹⁹F NMR (376 MHz, DMSO-d₆) δ (ppm) −57.62 (s), −194.20 (d, J=82.7 Hz), −196.08 (d, J=86.9 Hz). ³¹P NMR (162 MHz, DMSO-d₆) δ (ppm) 16.94 (d, J=82.4 Hz), 17.32 (d, J=87.0 Hz).

### Step E: ((S)-(2-(((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid

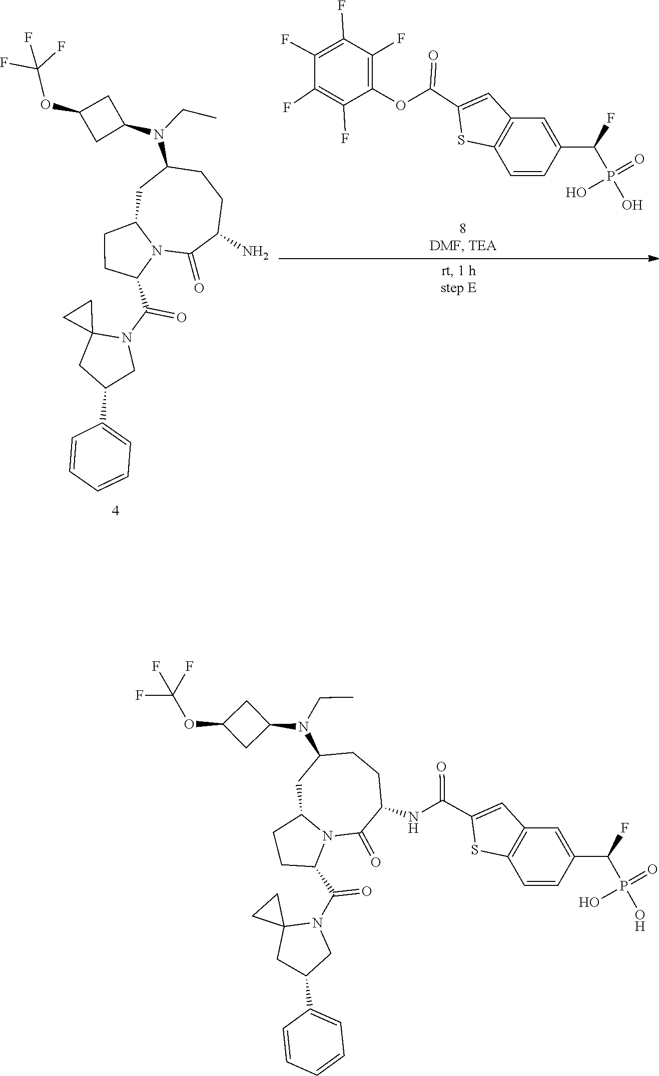 [SMILES: CCN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1 ; O=C(Oc1c(F)c(F)c(F)c(F)c1F)c1cc2cc([C@@H](F)P(=O)(O)O)ccc2s1 ; CCN([C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1)[C@H]1C[C@@H](OC(F)(F)F)C1]

To a solution of (3S,6S,9S,10aR)-6-amino-9-(ethyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one (4, 30 mg, TFA salt) in DMF (2 mL) was added TEA (13.7 mg, 0.135 mmol, 3 eq.), (S)-(fluoro(2-((perfluorophenoxy)carbonyl)benzo[b]thiophen-5-yl)methyl)phosphonic acid (8, 20 mg, 0.045 mmol, 1 eq.). The resulting mixture was stirred for 1 h at room temperature then the crude product was purified by Prep-HPLC to afford ((S)-(2-(((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A47, 9.4 mg, 0.011 mmol, 24.4%) as a white solid. LCMS (ESI): m/z=835.3 [M+H]⁺. 1H NMR (400 MHz, CD₃OD-d₄) δ (ppm) 8.08 (s, 1H), 8.07 (s, 1H), 7.95 (d, J=8.5 Hz, 1H), 7.62 (d, J=8.4 Hz, 1H), 7.45-7.36 (m, 2H), 7.36-7.28 (m, 2H), 7.28-7.19 (m, 1H), 5.80 (dd, J=44.6, 8.0 Hz, 1H), 4.86-4.77 (m, 2H), 4.75-4.48 (m, 3H), 4.19-3.90 (m, 3H), 3.84-3.53 (m, 2H), 3.28-2.93 (m, 3H), 2.91-2.81 (m, 1H), 2.75-2.60 (m, 1H), 2.57-2.48 (m, 1H), 2.46-2.35 (m, 1H), 2.35-2.18 (m, 4H), 2.17-1.72 (m, 7H), 1.70-1.58 (m, 1H), 1.51-1.34 (m, 3H), 0.71-0.41 (m, 2H). ¹⁹F NMR (377 MHz, CD₃OD-d₄) δ (ppm) −61.29 (s), −198.41 (d, J=82.6 Hz). ³¹P NMR (162 MHz, CD₃OD-d₄) δ (ppm) 12.06 (d, J=82.0 Hz).

### Synthesis of propyl (((S)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C101) and ((S)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A44)

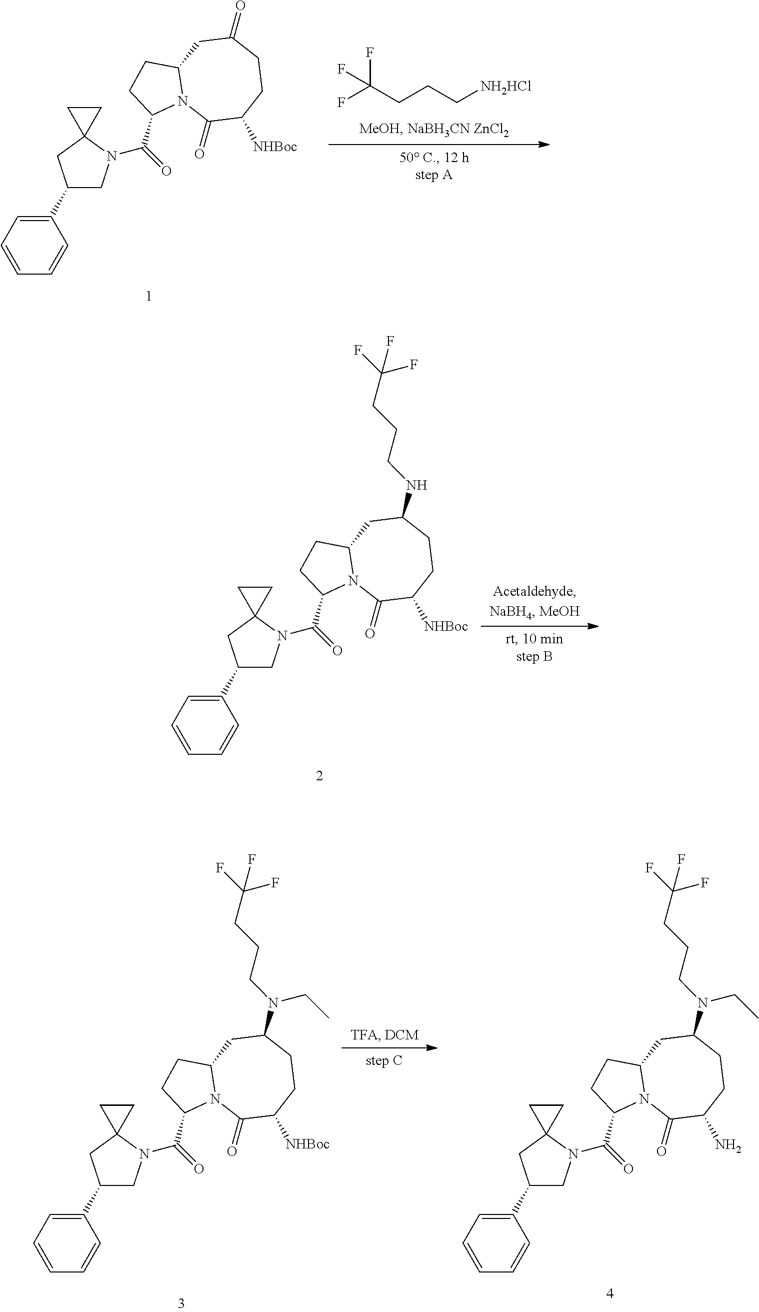

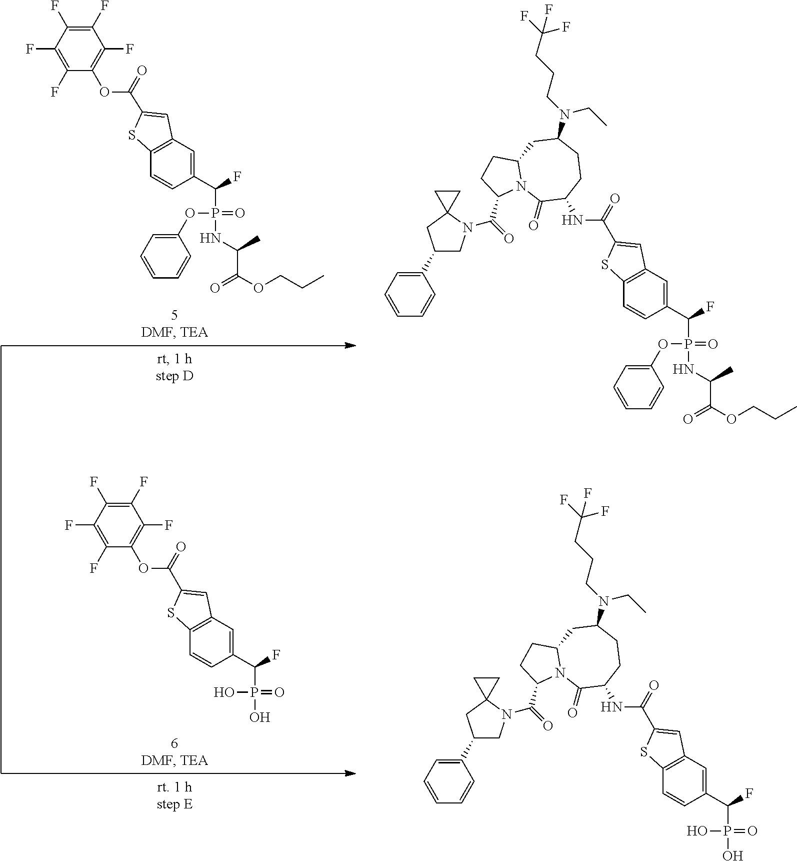 [SMILES: CCCOC(=O)[C@H](C)NP(=O)(Oc1ccccc1)[C@H](F)c1ccc2sc(C(=O)Oc3c(F)c(F)c(F)c(F)c3F)cc2c1 ; CCCOC(=O)[C@H](C)NP(=O)(Oc1ccccc1)[C@H](F)c1ccc2sc(C(=O)N[C@H]3CC[C@H](N(CC)CCCC(F)(F)F)C[C@H]4CC[C@@H](C(=O)N5C[C@H](c6ccccc6)CC56CC6)N4C3=O)cc2c1 ; O=C(Oc1c(F)c(F)c(F)c(F)c1F)c1cc2cc([C@@H](F)P(=O)(O)O)ccc2s1 ; CCN(CCCC(F)(F)F)[C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1]

### Step A: tert-butyl ((3S,6S,9S,10aR)-5-oxo-3-((S)-6-phenyl-4-azaspiro[2.4]heptane-4-carbonyl)-9-((4,4,4-trifluorobutyl)amino)decahydropyrrolo[1,2-a]azocin-6-yl)carbamate

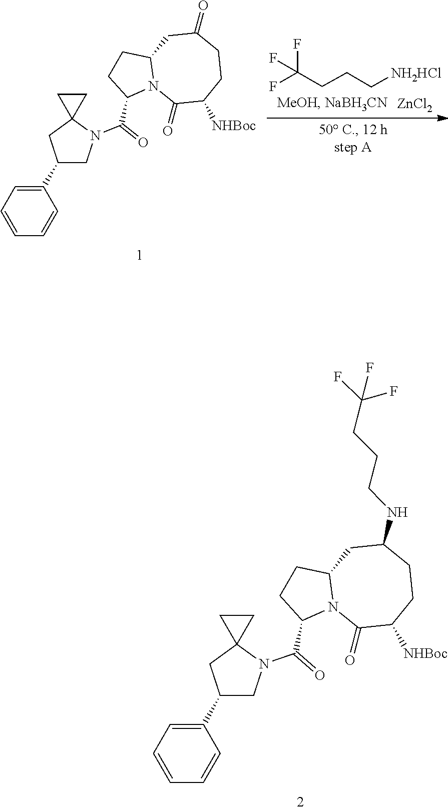

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (1, 1 g, 2.02 mmol, 1 eq.) in MeOH (20 mL) was added 4,4,4-trifluorobutan-1-amine hydrochloride (494 mg, 3.03 mml, 1.5 eq.), ZnCl₂ (2.75 g, 20.2 mmol, 10 eq.). The mixture was stirred for 1 h at 50° C., then NaBH₃CN (1.27 g, 20.2 mmol, 10 eq.) was added portion wise every 30 mins (2 eq. (254 mg each time) added 5 times). The mixture was stirred for 12 h at 50° C., then cooled to room temperature and used directly in the next step without further workup. LCMS (ESI): m/z=607 [M+H]⁺.

### Step B: tert-butyl ((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamate

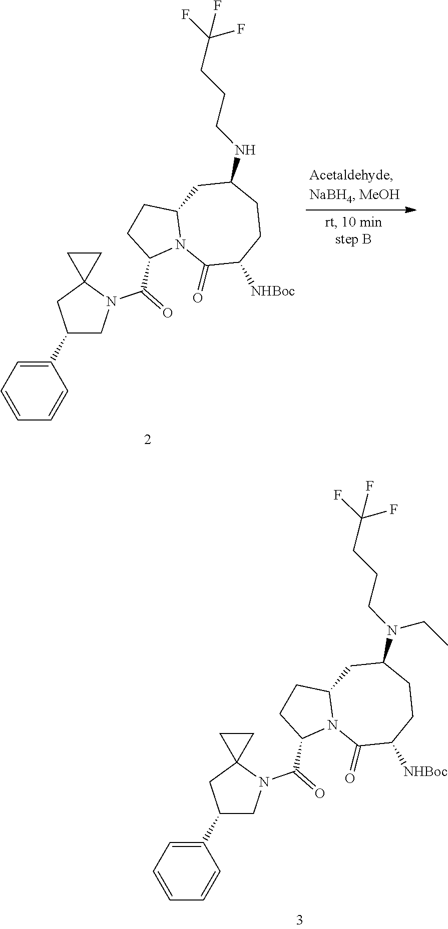 [SMILES: CC(C)(C)OC(=O)N[C@H]1CC[C@H](NCCCC(F)(F)F)C[C@H]2CC[C@@H](C(=O)N3C[C@H](c4ccccc4)CC34CC4)N2C1=O ; CCN(CCCC(F)(F)F)[C@H]1CC[C@H](NC(=O)OC(C)(C)C)C(=O)N2[C@H](CC[C@H]2C(=O)N2C[C@H](c3ccccc3)CC23CC3)C1]

To the above mixture (step A) was added 40% acetaldehyde (1 mL) at room temperature, and stirred for 10 min, then NaBH₄ (767 mg, 20.2 mmol, 10 eq.) was added to the mixture, stirred for additional 10 min, then the reaction was quenched by NH₄Cl (sat. aq), diluted with H₂O (100 mL), then extracted with EtOAc (50 mL×3). The organic layers were combined and washed with brine (20 mL), dried over Na₂SO₄, then concentrated to dryness under reduced pressure. The crude product was purified by C18 column (MeCN:H₂O (0.1% ammonium hydroxide)=5%-70%) to afford tert-butyl ((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamate (3, 650 mg, 1.02 mmol, 50.5% yield, two steps) as a white solid. LCMS (ESI): m/z=635 [M+H]⁺.

### Step C: (3S,6S,9S,10aR)-6-amino-9-(ethyl(4,4,4-trifluorobutyl)amino)-3-((S)-6-phenyl-4-azaspiro[2.4]heptane-4-carbonyl)octahydropyrrolo[1,2-a]azocin-5(1H)-one

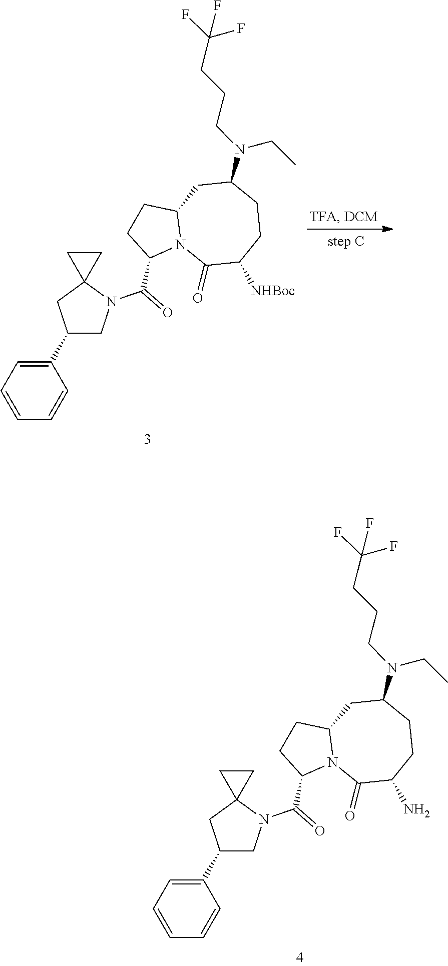 [SMILES: CCN(CCCC(F)(F)F)[C@H]1CC[C@H](NC(=O)OC(C)(C)C)C(=O)N2[C@H](CC[C@H]2C(=O)N2C[C@H](c3ccccc3)CC23CC3)C1 ; CCN(CCCC(F)(F)F)[C@H]1CC[C@H](N)C(=O)N2[C@H](CC[C@H]2C(=O)N2C[C@H](c3ccccc3)CC23CC3)C1]

To a solution of tert-butyl ((3S,6S,9S,10aR)-9-(methyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamate (3, 120 mg, 0.188 mmol, 1 eq.) in DCM (2 mL) was added TFA (2 mL) and the resulting mixture was stirred for 1 h at room temperature. The resulting mixture was concentrated to dryness under reduced pressure to afford crude product (3S,6S,9S,10aR)-6-amino-9-(ethyl(4,4,4-trifluorobutyl)amino)-3-((S)-6-phenyl-4-azaspiro[2.4]heptane-4-carbonyl)octahydropyrrolo[1,2-a]azocin-5(1H)-one (4, 120 mg, TFA salt) as a yellow oil which used in the next step without further purification. LCMS (ESI): m/z=535 [M+H]⁺.

### Step D: propyl (((S)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate

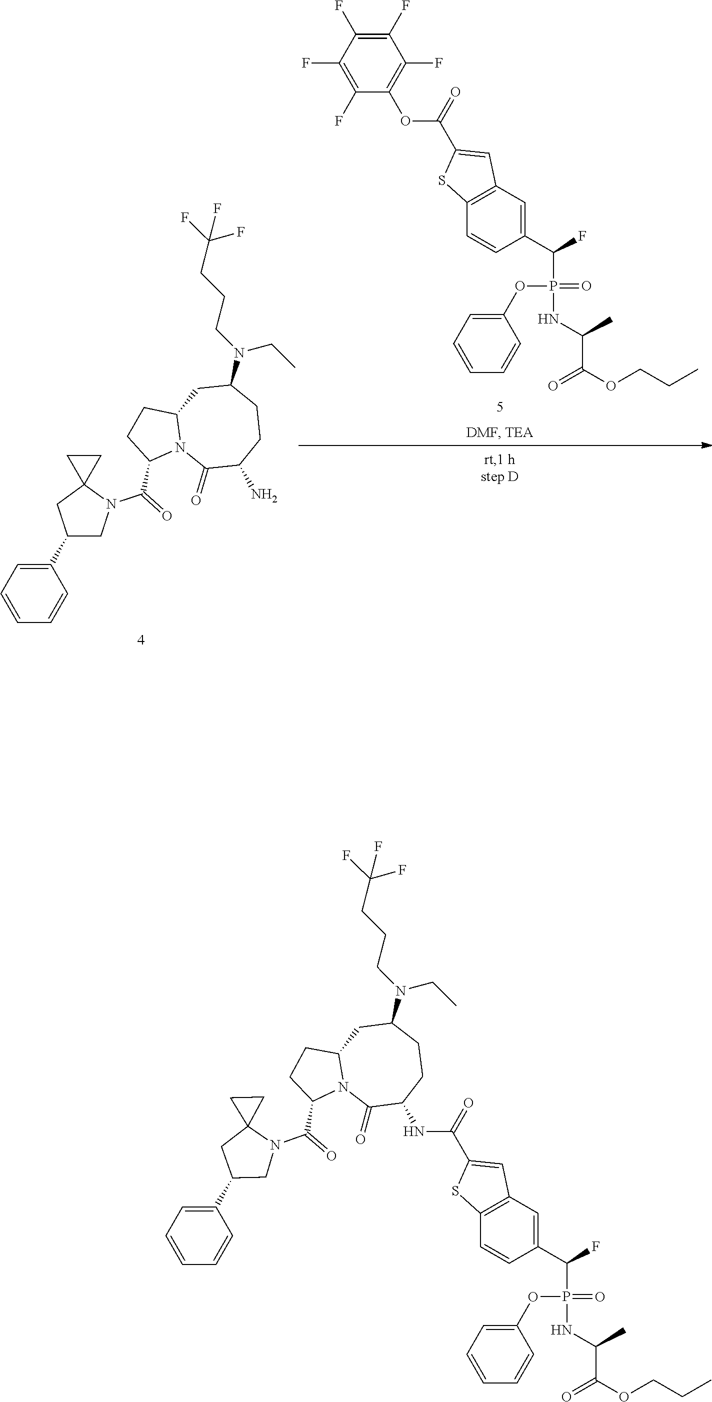 [SMILES: CCN(CCCC(F)(F)F)[C@H]1CC[C@H](N)C(=O)N2[C@H](CC[C@H]2C(=O)N2C[C@H](c3ccccc3)CC23CC3)C1 ; CCCOC(=O)[C@H](C)NP(=O)(Oc1ccccc1)[C@H](F)c1ccc2sc(C(=O)Oc3c(F)c(F)c(F)c(F)c3F)cc2c1 ; CCCOC(=O)[C@H](C)NP(=O)(Oc1ccccc1)[C@H](F)c1ccc2sc(C(=O)N[C@H]3CC[C@H](N(CC)CCCC(F)(F)F)C[C@H]4CC[C@@H](C(=O)N5C[C@H](c6ccccc6)CC56CC6)N4C3=O)cc2c1]

To a solution of (3S,6S,9S,10aR)-6-amino-9-(ethyl(4,4,4-trifluorobutyl)amino)-3-((S)-6-phenyl-4-azaspiro[2.4]heptane-4-carbonyl)octahydropyrrolo[1,2-a]azocin-5(1H)-one (4, 30 mg, TFA salt) in DMF (2 mL) was added TEA (14.3 mg, 0.141 mmol, 3 eq.), perfluorophenyl 5-((1S)-fluoro((((S)-1-oxo-1-propoxypropan-2-yl)amino)(phenoxy)phosphoryl)methyl)benzo[b]thiophene-2-carboxylate (5, 30 mg, 0.046 mmol, 1 eq). The resulting mixture was stirred for 1 h at room temperature, LCMS show that the reaction was completed. The crude product was purified by Prep-HPLC to afford propyl (((S)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C101, 25 mg, 0.025 mmol, 54.3%) as a white solid. LCMS (ESI): m/z=996.6 [M+H]⁺. 1H NMR (400 MHz, DMSO-d₆) δ (ppm) 8.83-8.74 (m, 1H), 8.29-8.24 (m, 1H), 8.11-8.01 (m, 2H), 7.65-7.55 (m, 1H), 7.39-7.27 (m, 6H), 7.24-7.11 (m, 4H), 6.31-6.08 (m, 2H), 4.78-4.67 (m, 1H), 4.51-4.43 (m, 1H), 4.35-4.25 (m, 1H), 4.16-4.06 (m, 1H), 3.95-3.65 (m, 4H), 3.58-3.48 (m, 1H), 3.07-2.96 (m, 1H), 2.48-2.16 (m, 8H), 2.15-2.08 (m, 1H), 2.04-1.92 (m, 3H), 1.85-1.43 (m, 12H), 1.20-0.92 (m, 6H), 0.84-0.77 (m, 3H), 0.53-0.38 (m, 2H). ¹⁹F NMR (376 MHz, DMSO d₆) δ (ppm) −64.51 (d, J=2.7 Hz), −194.17 (d, J=83.3 Hz), −196.05 (d, J=87.0 Hz). ³¹P NMR (162 MHz, DMSO-d₆) δ (ppm) 17.33 (d, J=86.5 Hz), 16.94 (d, J=82.4 Hz).

### Step E: ((S)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid

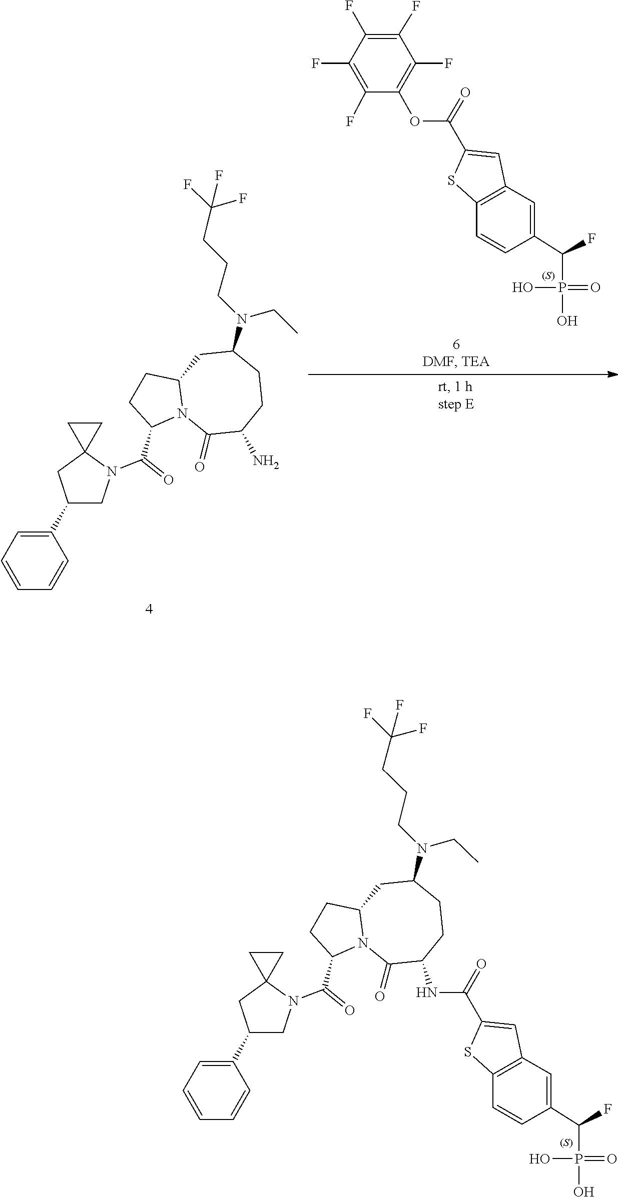

To a solution of (3S,6S,9S,10aR)-6-amino-9-(ethyl(4,4,4-trifluorobutyl)amino)-3-((S)-6-phenyl-4-azaspiro[2.4]heptane-4-carbonyl)octahydropyrrolo[1,2-a]azocin-5(1H)-one (4, 30 mg, TFA salt) in DMF (2 mL) was added TEA (14.3 mg, 0.141 mmol, 3 eq.), (S)-(fluoro(2-((perfluorophenoxy)carbonyl)benzo[b]thiophen-5-yl)methyl)phosphonic acid (6, 21 mg, 0.046 mmol, 1 eq). The resulting mixture was stirred for 1 h at room temperature, then the crude product was purified by Prep-HPLC to afford propyl ((S)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A44, 10 mg, 0.012 mmol, 26.1%) as a white solid. LCMS (ESI): m/z=807.3 [M+H]⁺. ¹H NMR (400 MHz, DMSO-d₆) δ (ppm) 8.97 (s, 1H), 8.86-8.78 (m, 1H), 8.24 (d, J=4.2 Hz, 1H), 8.03 (d, J=8.5 Hz, 1H), 7.98 (s, 1H), 7.53 (d, J=8.6 Hz, 1H), 7.38-7.28 (m, 4H), 7.26-7.19 (m, 1H), 5.93-5.73 (m, 1H), 4.86-4.78 (m, 1H), 4.59-4.50 (m, 2H), 4.17-4.10 (m, 1H), 3.88-3.72 (m, 2H), 3.56-3.51 (m, 1H), 3.22-3.06 (m, 4H), 2.49-2.30 (m, 3H), 2.26-2.19 (m, 1H), 2.14-1.73 (m, 13H), 1.58-1.48 (m, 1H), 1.34-1.24 (m, 3H), 0.58-0.41 (m, 2H). ¹⁹F NMR (376 MHz, DMSO-d₆) δ (ppm) −64.75 (s), −194.71 (d, J=81.7 Hz). ³¹P NMR (162 MHz, DMSO-d₆) δ (ppm) 10.95 (d, J=80.8 Hz).

### Synthesis of ((R)-(2-(((3S,6S,9S,10aR)-9-(((1s,3R)-3-(difluoromethyl)cyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A35) and propyl ((S)-((R)-(2-(((3S,6S,9S,10aR)-9-(((1s,3R)-3-(difluoromethyl)cyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C71)

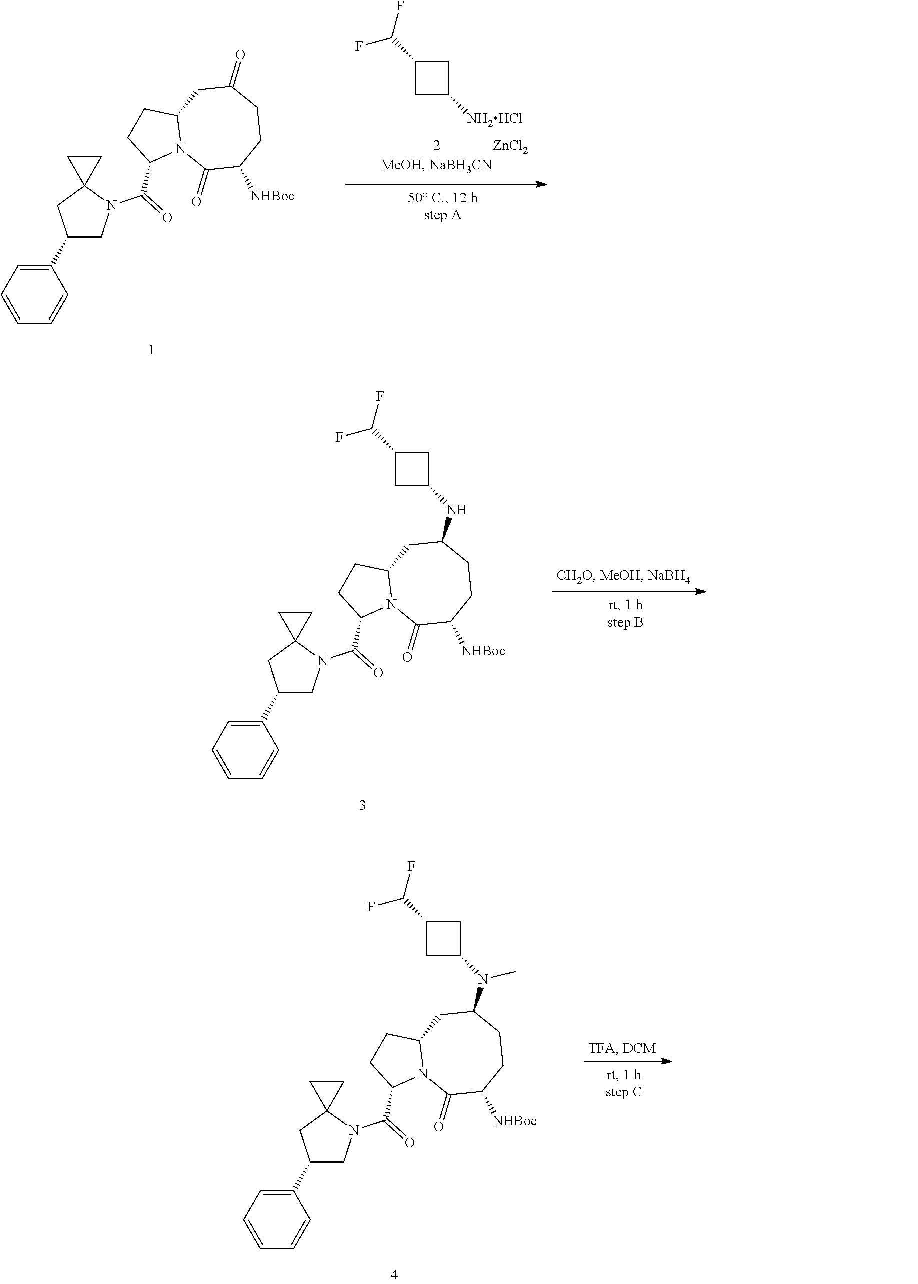

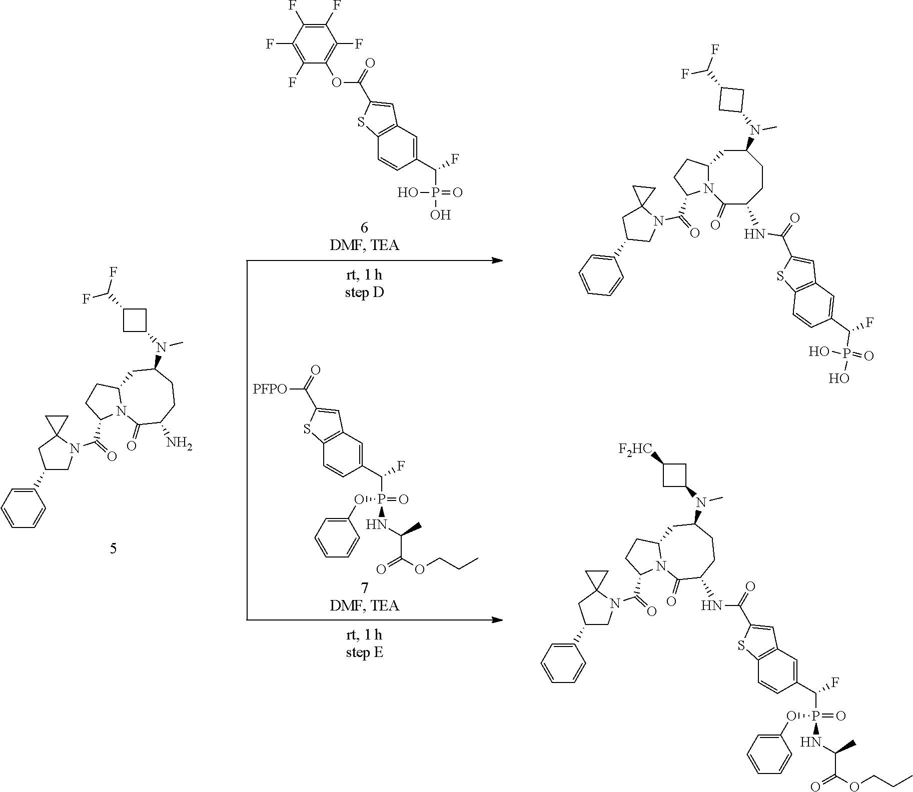

### Step A: tert-butyl ((3S,6S,9S,10aR)-9-(((1s,3R)-3-(difluoromethyl)cyclobutyl)amino)-5-oxo-3-((S)-6-phenyl-4-azaspiro[2.4]heptane-4-carbonyl)decahydropyrrolo[1,2-a]azocin-6-yl)carbamate

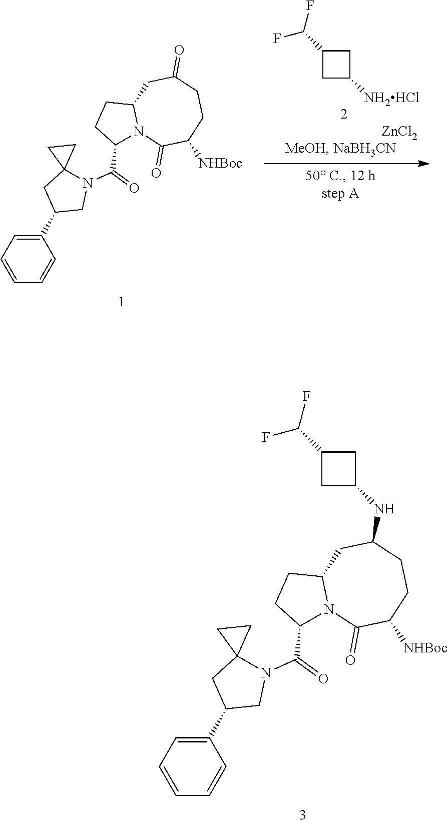

To a solution of tert-butyl ((3S,6S,10aR)-5,9-dioxo-3-((R)-6-phenyl-4-azaspiro[2.4]heptane-4-carbonyl)decahydropyrrolo[1,2-a]azocin-6-yl)carbamate (1, 3 g, 6.06 mmol, 1 eq.) in MeOH (60 mL) was added (1s,3s)-3-(trifluoromethyl)cyclobutan-1-amine hydrochloride (2, 1.43 g, 9.09 mmol, 1.5 eq.), ZnCl₂ (8.3 g, 60.6 mmol, 10 eq.), the solution was stirred for 2 hr at 50° C. under the atmosphere of N₂. Then NaBH₃CN (3.81 g, 60.6 mmol, 10 eq.) was added portion wise every 30 mins (2 eq. (762 mg each time) added 5 times). The solution was stirred for 12 hr at 50° C., then cooled to room temperature and was used to next step without further workup. LCMS (ESI): m/z=601 [M+H]⁺.

### Step B: tert-butyl ((3S,6S,9S,10aR)-9-(((1s,3R)-3-(difluoromethyl)cyclobutyl)(methyl)amino)-5-oxo-3-((S)-6-phenyl-4-azaspiro[2.4]heptane-4-carbonyl)decahydropyrrolo[1,2-a]azocin-6-yl)carbamate

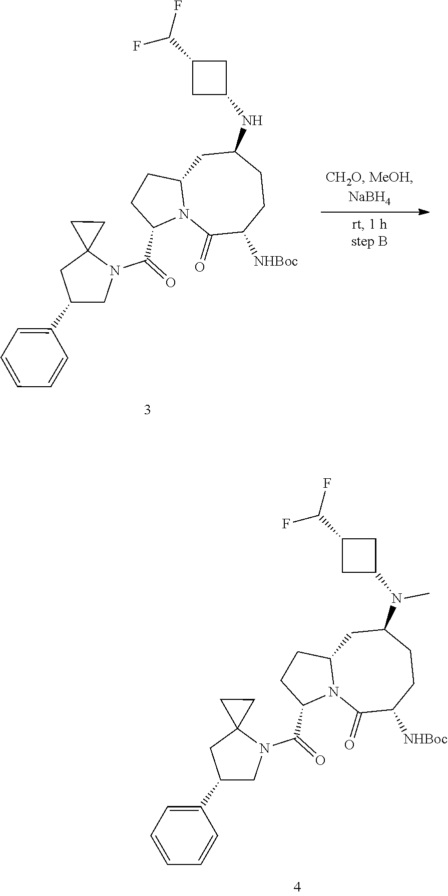

To the above mixture (step A) was added 40% formaldehyde aqueous (3 mL) at room temperature, the mixture was stirred for 30 min, then NaBH₄ (1.15 g, 30.3 mmol, 5 eq.) was added in portions, the mixture was stirred for another 30 min, then reaction was quenched by NH₄Cl (sat. aq), diluted with H₂O (200 mL), then extracted with EtOAc (80 mL×3). The organic layers were combined and washed with brine (20 mL), dried over Na₂SO₄, then concentrated to dryness under reduced pressure. The crude product was purified by C18 column (MeCN:H₂O (0.1% ammonium hydroxide)=5%-70%) to afford tert-butyl ((3S,6S,9S,10aR)-9-(((1s,3R)-3-(difluoromethyl)cyclobutyl)(methyl)amino)-5-oxo-3-((S)-6-phenyl-4-azaspiro[2.4]heptane-4-carbonyl)decahydropyrrolo[1,2-a]azocin-6-yl)carbamate (4, 3 g, 4.86 mmol, 78.9% yield two steps) as a white solid. LCMS (ESI): m/z=615 [M+H]⁺.

### Step C: (3S,6S,9S,10aR)-6-amino-9-(((1s,3R)-3-(difluoromethyl)cyclobutyl)(methyl)amino)-3-((S)-6-phenyl-4-azaspiro[2.4]heptane-4-carbonyl)octahydropyrrolo[1,2-a]azocin-5(1H)-one

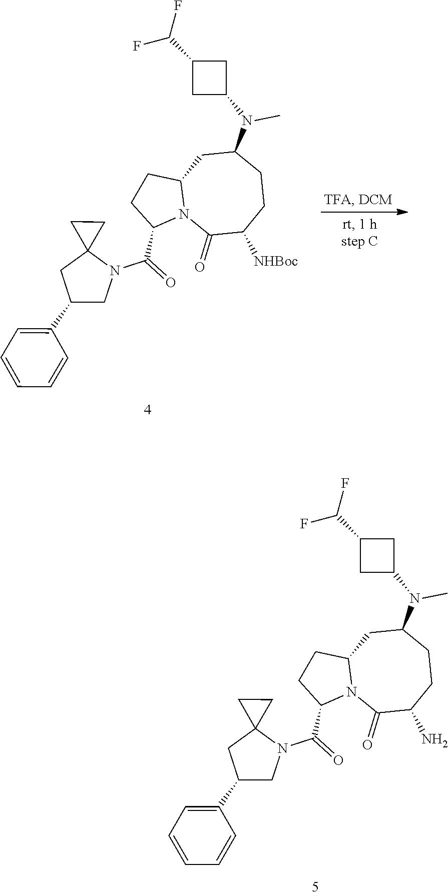

To a solution of tert-butyl ((3S,6S,9S,10aR)-9-(((1s,3R)-3-(difluoromethyl)cyclobutyl)(methyl)amino)-5-oxo-3-((S)-6-phenyl-4-azaspiro[2.4]heptane-4-carbonyl)decahydropyrrolo[1,2-a]azocin-6-yl)carbamate (4, 30 mg, 0.047 mmol) in DCM (2 mL) was added TFA (2 mL). The resulting mixture was stirred for 1 h at room temperature, then the resulting mixture was concentrated to dryness under reduced pressure to afford crude product (3S,6S,9S,10aR)-6-amino-9-(((1s,3R)-3-(difluoromethyl)cyclobutyl)(methyl)amino)-3-((S)-6-phenyl-4-azaspiro[2.4]heptane-4-carbonyl)octahydropyrrolo[1,2-a]azocin-5(1H)-one (5, 30 mg, TFA salt) as a yellow oil which was used to the next step without further purification. LCMS (ESI): m/z=515 [M+H]⁺.

### Step D: ((R)-(2-(((3S,6S,9S,10aR)-9-(((1s,3R)-3-(difluoromethyl)cyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid

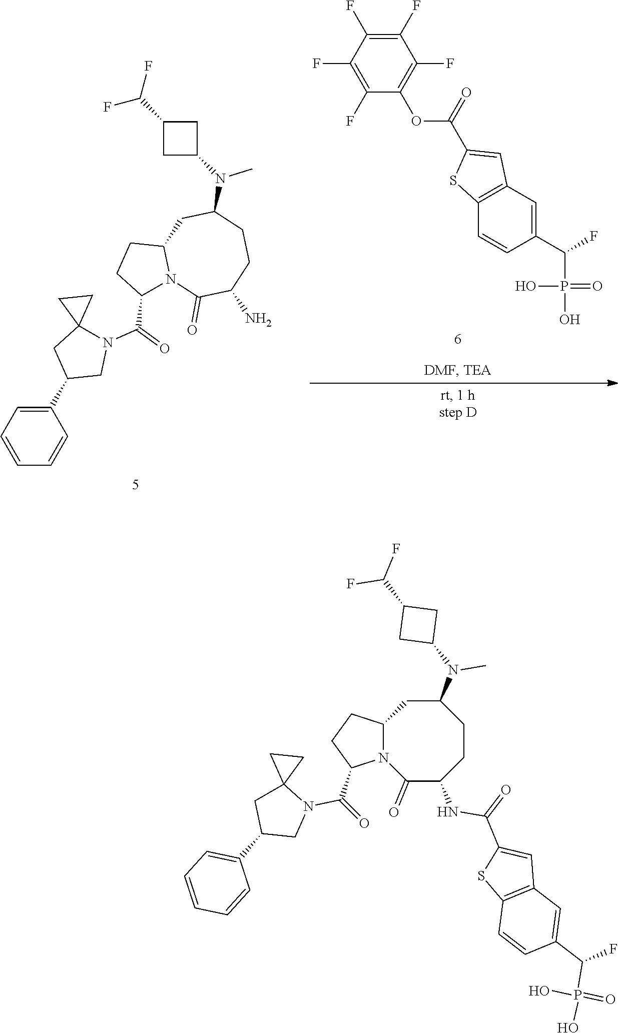 [SMILES: CN([C@H]1CC[C@H](N)C(=O)N2[C@H](CC[C@H]2C(=O)N2C[C@H](c3ccccc3)CC23CC3)C1)[C@H]1C[C@@H](C(F)F)C1 ; O=C(Oc1c(F)c(F)c(F)c(F)c1F)c1cc2cc([C@H](F)P(=O)(O)O)ccc2s1 ; CN([C@H]1CC[C@H](NC(=O)c2cc3cc([C@H](F)P(=O)(O)O)ccc3s2)C(=O)N2[C@H](CC[C@H]2C(=O)N2C[C@H](c3ccccc3)CC23CC3)C1)[C@H]1C[C@@H](C(F)F)C1]

To a solution of (3S,6S,9S,10aR)-6-amino-9-(((1s,3R)-3-(difluoromethyl)cyclobutyl)(methyl)amino)-3-((S)-6-phenyl-4-azaspiro[2.4]heptane-4-carbonyl)octahydropyrrolo[1,2-a]azocin-5(1H)-one (5, 30 mg, TFA salt) in DMF (2 mL) was added TEA (9.9 mg, 0.098 mmol, 2 eq.), (R)-(fluoro(2-((perfluorophenoxy)carbonyl)benzo[b]thiophen-5-yl)methyl)phosphonic acid (6, 21.3 mg, 0.049 mmol, 1 eq.). The resulting mixture was stirred for 1 h at room temperature, then the crude product was purified by Prep-HPLC to afford ((R)-(2-(((3S,6S,9S,10aR)-9-(((1s,3R)-3-(difluoromethyl)cyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A35, 17 mg) as a white solid. LCMS (ESI): m/z=787 [M+H]⁺. 1H NMR (400 MHz, DMSO-d₆) δ (ppm) 9.74-9.31 (m, 1H), 8.89-8.69 (m, 1H), 8.29-8.20 (m, 1H), 8.08-7.94 (m, 2H), 7.57-7.49 (m, 1H), 7.38-7.28 (m, 4H), 7.26-7.21 (m, 1H), 6.23-5.74 (m, 2H), 4.85-4.75 (m, 1H), 4.62-4.46 (m, 2H), 4.20-4.07 (m, 1H), 3.87-3.74 (m, 2H), 3.60-3.49 (m, 2H), 2.62-2.53 (m, 3H), 2.47-1.49 (m, 19H), 0.61-0.40 (m, 2H). ¹⁹F NMR (376 MHz, DMSO-d₆) δ (ppm)-122.05-−122.83 (m), −194.81-−195.03 (m). ³¹P NMR (162 MHz, DMSO-d₆) δ (ppm) 11.22-10.71 (m).

### Step E: propyl ((S)-((R)-(2-(((3S,6S,9S,10aR)-9-(((1s,3R)-3-(difluoromethyl)cyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate

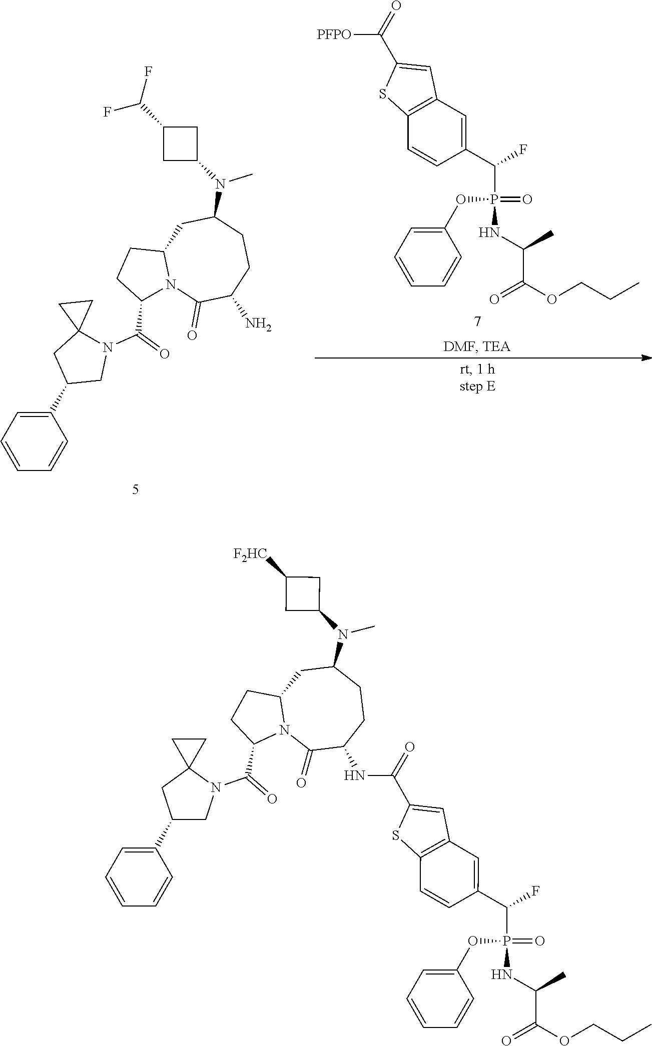

To a solution of (3S,6S,9S,10aR)-6-amino-9-(((1s,3R)-3-(difluoromethyl)cyclobutyl)(methyl)amino)-3-((S)-6-phenyl-4-azaspiro[2.4]heptane-4-carbonyl)octahydropyrrolo[1,2-a]azocin-5(1H)-one (5, 30 mg, TFA salt) in DMF (2 mL) was added TEA (9.9 mg, 0.098 mmol, 2 eq.), perfluorophenyl 5-((R)-fluoro((S)-(((S)-1-oxo-1-propoxypropan-2-yl)amino)(phenoxy)phosphoryl)methyl)benzo[b]thiophene-2-carboxylate (7, 31.5 mg, 0.049 mmol, 1 eq.). The resulting mixture was stirred for 1 h at room temperature. The crude product was purified by Prep-HPLC to afford propyl ((S)-((R)-(2-(((3S,6S,9S,10aR)-9-(((1s,3R)-3-(difluoromethyl)cyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C71, 17 mg) as a white solid. LCMS (ESI): m/z=976 [M+H]⁺. ¹H NMR (400 MHz, DMSO-dd) δ (ppm) 8.81 (d, J=7.4 Hz, 1H), 8.27 (s, 1H), 8.11-8.04 (m, 2H), 7.60 (d, J=8.5 Hz, 1H), 7.39-7.27 (m, 6H), 7.25-7.14 (m, 4H), 6.29-5.82 (m, 3H), 4.81-4.66 (m, 1H), 4.50 (t, J=8.5 Hz, 1H), 4.43-4.29 (m, 1H), 4.13 (t, J=8.6 Hz, 1H), 3.99-3.88 (m, 2H), 3.83-3.68 (m, 2H), 3.60-3.46 (m, 1H), 3.41-3.33 (m, 2H), 2.44-1.44 (m, 24H), 1.13 (d, J=7.1 Hz, 3H), 0.83 (t, J=7.4 Hz, 3H), 0.56-0.39 (m, 2H). ¹⁹F NMR (376 MHz, DMSO-d₆) δ (ppm) −121.32 (s), −196.32 (d, J=85.8 Hz). ³¹P NMR (162 MHz, DMSO-d₆) δ (ppm) 17.08 (d, J=86.2 Hz).

### Synthesis of propyl (((R)-fluoro(2-(((3S,6S,9S,10aR)-9-(methyl((R)-tetrahydrofuran-3-yl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C24) and ((R)-fluoro(2-(((3S,6S,9S,10aR)-9-(methyl((R)-tetrahydrofuran-3-yl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A19)

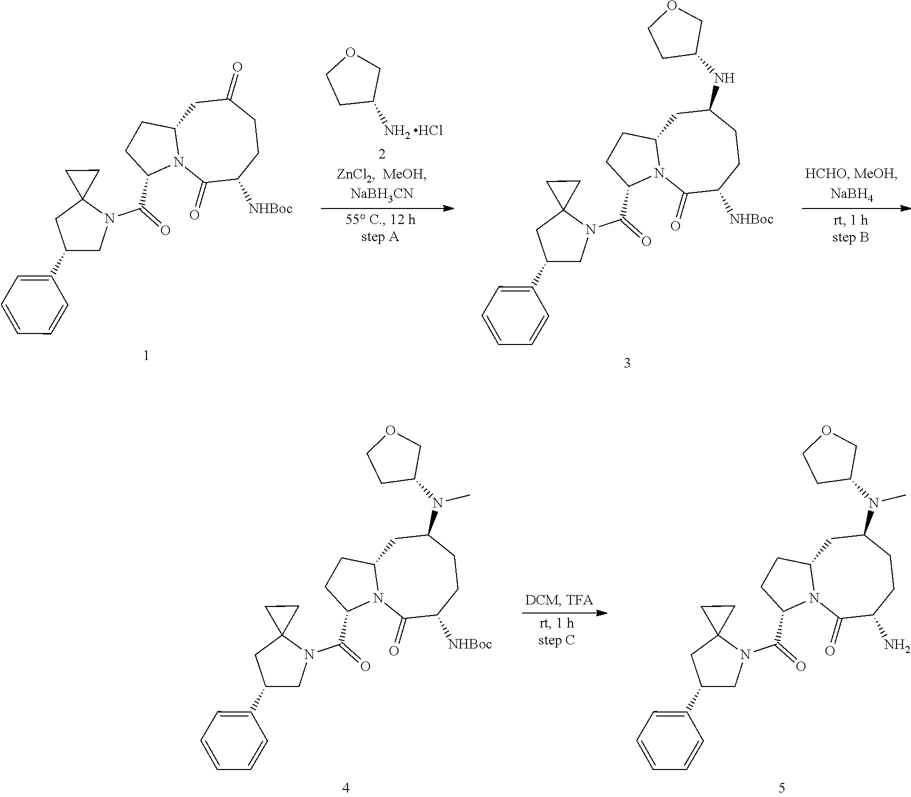 [SMILES: CC(C)(C)OC(=O)N[C@H]1CCC(=O)C[C@H]2CC[C@@H](C(=O)N3C[C@H](c4ccccc4)CC34CC4)N2C1=O ; N[C@@H]1CCOC1 ; Cl ; CC(C)(C)OC(=O)N[C@H]1CC[C@H](N[C@@H]2CCOC2)C[C@H]2CC[C@@H](C(=O)N3C[C@H](c4ccccc4)CC34CC4)N2C1=O ; CN([C@@H]1CCOC1)[C@H]1CC[C@H](NC(=O)OC(C)(C)C)C(=O)N2[C@H](CC[C@H]2C(=O)N2C[C@H](c3ccccc3)CC23CC3)C1 ; CN([C@@H]1CCOC1)[C@H]1CC[C@H](N)C(=O)N2[C@H](CC[C@H]2C(=O)N2C[C@H](c3ccccc3)CC23CC3)C1]

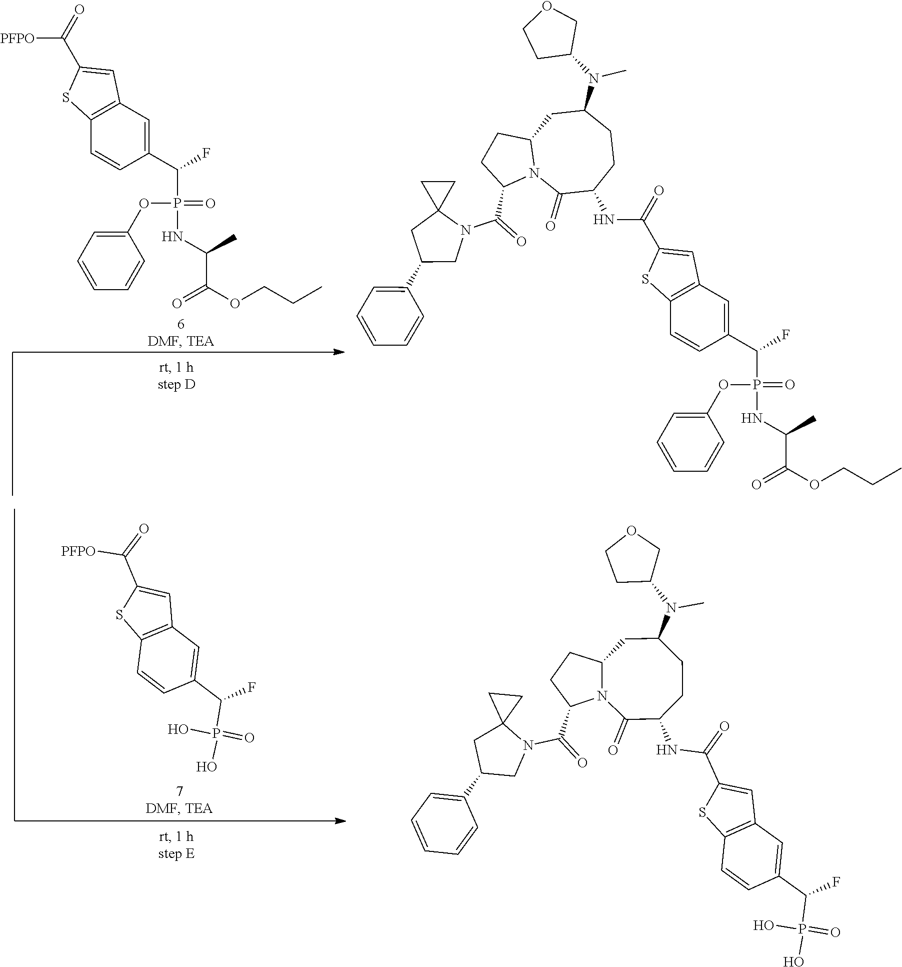

### Step A: tert-butyl ((3S,6S,9S,10aR)-5-oxo-3-((S)-6-phenyl-4-azaspiro[2.4]heptane-4-carbonyl)-9-(((R)-tetrahydrofuran-3-yl)amino)decahydropyrrolo[1,2-a]azocin-6-yl)carbamate

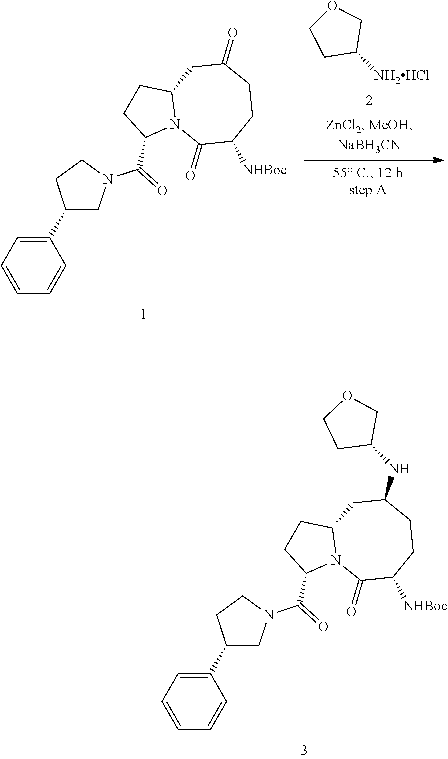

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (1, 1 g, 2.02 mmol, 1 eq.) in MeOH (20 mL) was added (R)-tetrahydrofuran-3-amine hydrochloride (2, 372.7 mg, 3.03 mmol, 1.5 eq.), ZnCl₂ (2.75 g, 20.2 mmol, 10 eq.). The resulting mixture was stirred for 1 h at 50° C., then NaBH₃CN (1.27 g, 20.2 mmol, 10 eq.) was added portion wise every 30 mins (2 eq. (254 mg each time) added 5 times). The mixture was stirred for 12 h at 50° C., LCMS show that the reaction was completed. Then cooled to room temperature, the mixture was used to the next step directly without further workup. LCMS (ESI): m/z=567 [M+H]⁺.

### Step B: tert-butyl ((3S,6S,9S,10aR)-9-(methyl((R)-tetrahydrofuran-3-yl)amino)-5-oxo-3-((S)-6-phenyl-4-azaspiro[2.4]heptane-4-carbonyl)decahydropyrrolo[1,2-a]azocin-6-yl)carbamate

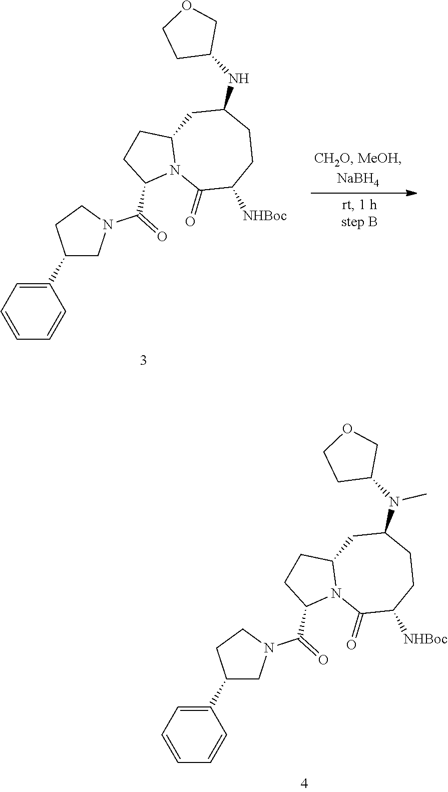 [SMILES: CC(C)(C)OC(=O)N[C@H]1CC[C@H](N[C@@H]2CCOC2)C[C@H]2CC[C@@H](C(=O)N3CC[C@@H](c4ccccc4)C3)N2C1=O ; CN([C@@H]1CCOC1)[C@H]1CC[C@H](NC(=O)OC(C)(C)C)C(=O)N2[C@H](CC[C@H]2C(=O)N2CC[C@@H](c3ccccc3)C2)C1]

To a above mixture (step A) was added 40% formaldehyde (1 mL) at room temperature, then the mixture was stirred for 10 min, then NaBH₄ (767 mg, 20.2 mmol, 10 eq.) was added to the mixture, stirred for another 10 min, LCMS show that the reaction was completed, the reaction was quenched by NH₄Cl (sat. aq), diluted with H₂O (80 mL), then extracted with EtOAc (40 mL×3), the organic layers were combined and washed with brine (20 mL), dried over Na₂SO₄, filtered, then concentrated to dryness under reduced pressure. The crude product was purified by C18 column (MeCN:H₂O (0.1% ammonium hydroxide)=5%-70%) to afford tert-butyl ((3S,6S,9S,10aR)-9-(methyl((R)-tetrahydrofuran-3-yl)amino)-5-oxo-3-((S)-6-phenyl-4-azaspiro[2.4]heptane-4-carbonyl)decahydropyrrolo[1,2-a]azocin-6-yl)carbamate (4, 480 mg, 0.826 mmol, 40.9% yield, two steps) as a white solid. LCMS (ESI): m/z=581 [M+H]⁺.

### Step C: (3S,6S,9S,10aR)-6-amino-9-(methyl((R)-tetrahydrofuran-3-yl)amino)-3-((S)-6-phenyl-4-azaspiro[2.4]heptane-4-carbonyl)octahydropyrrolo[1,2-a]azocin-5(1H)-one

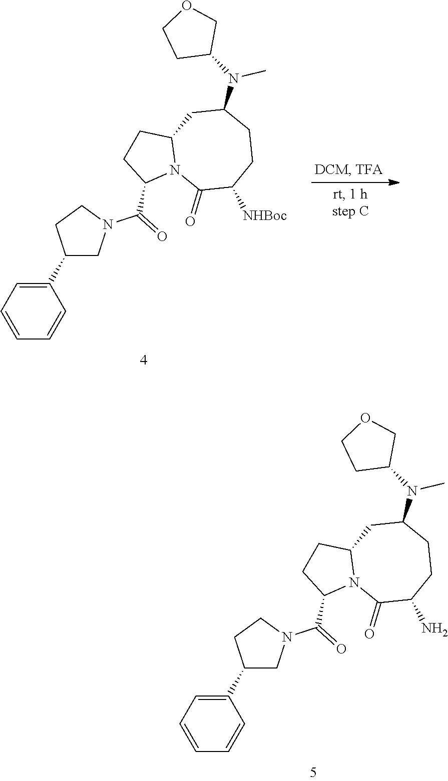 [SMILES: CN([C@@H]1CCOC1)[C@H]1CC[C@H](NC(=O)OC(C)(C)C)C(=O)N2[C@H](CC[C@H]2C(=O)N2CC[C@@H](c3ccccc3)C2)C1 ; CN([C@@H]1CCOC1)[C@H]1CC[C@H](N)C(=O)N2[C@H](CC[C@H]2C(=O)N2CC[C@@H](c3ccccc3)C2)C1]

To a solution of tert-butyl ((3S,6S,9S,10aR)-9-(methyl((R)-tetrahydrofuran-3-yl)amino)-5-oxo-3-((S)-6-phenyl-4-azaspiro[2.4]heptane-4-carbonyl)decahydropyrrolo[1,2-a]azocin-6-yl)carbamate (4, 40 mg, 0.069 mmol, 1 eq.) in DCM (2 mL) was added TFA (2 mL), The resulting mixture was stirred for 1 hr at room temperature. The resulting mixture was concentrated to dryness under reduced pressure to afford crude product (3S,6S,9S,10aR)-6-amino-9-(methyl((R)-tetrahydrofuran-3-yl)amino)-3-((S)-6-phenyl-4-azaspiro[2.4]heptane-4-carbonyl)octahydropyrrolo[1,2-a]azocin-5(1H)-one (5, 40 mg, TFA salt) as a yellow oil which used in the next step without further purification. LCMS (ESI): m/z=481 [M+H]⁺.

### Step D: propyl (((R)-fluoro(2-(((3S,6S,9S,10aR)-9-(methyl((R)-tetrahydrofuran-3-yl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

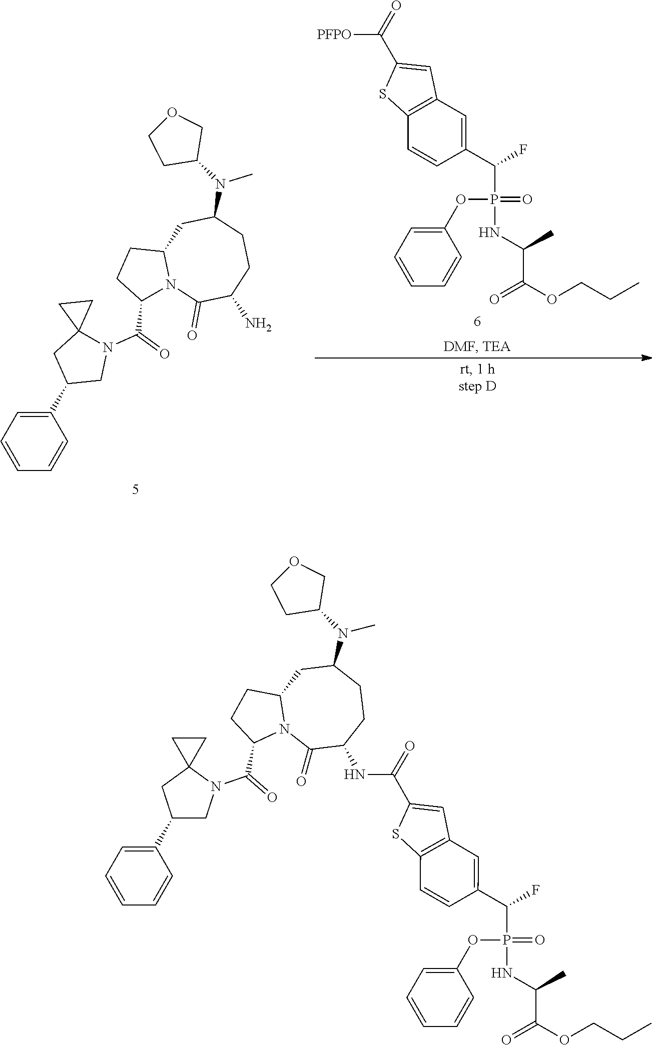

To a solution of (3S,6S,9S,10aR)-6-amino-9-(methyl((R)-tetrahydrofuran-3-yl)amino)-3-((S)-6-phenyl-4-azaspiro[2.4]heptane-4-carbonyl)octahydropyrrolo[1,2-a]azocin-5(1H)-one (5, 20 mg, TFA salt) in DMF (2 mL) was added TEA (10.4 mg, 0.103 mmol, 3 eq.), perfluorophenyl 5-((1R)-fluoro((((S)-1-oxo-1l-propoxypropan-2-yl)amino)(phenoxy)phosphoryl)methyl)benzo[b]thiophene-2-carboxylate (6, 22.2 mg, 0.034 mmol, 1 eq). The resulting mixture was stirred for 1 h at room temperature, then the crude product was purified by Prep-HPLC to afford propyl (((R)-fluoro(2-(((3S,6S,9S,10aR)-9-(methyl((R)-tetrahydrofuran-3-yl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C24, 21 mg, 0.022 mmol, 64.7%) as a white solid. LCMS (ESI): m/z=942.6 [M+H]⁺. ¹H NMR (400 MHz, DMSO-d₆) δ (ppm) 8.83-8.73 (m, 1H), 8.27 (s, 1H), 8.09-8.02 (m, 2H), 7.64-7.56 (m, 1H), 7.38-7.26 (m, 6H), 7.24-7.13 (m, 4H), 6.28-6.05 (m, 2H), 4.77-4.66 (m, 1H), 4.47 (t, J=8.6 Hz, 1H), 4.34-4.23 (m, 1H), 4.12 (t, J=8.5 Hz, 1H), 3.98-3.89 (m, 1H), 3.86-3.60 (m, 6H), 3.59-3.47 (m, 2H), 3.17-3.06 (m, 1H), 2.99-2.87 (m, 1H), 2.40-2.30 (m, 1H), 2.23 (t, J=11.5 Hz, 1H), 2.16-2.09 (m, 2H), 2.07 (s, 3H), 2.03-1.87 (m, 4H), 1.85-1.48 (m, 9H), 1.47-1.36 (m, 1H), 1.17-1.09 (m, 3H), 0.85-0.75 (m, 3H), 0.54-0.41 (m, 2H). ¹⁹F NMR (376 MHz, DMSO-d₆) δ (ppm) −194.29 (d, J=82.5 Hz), −196.30 (d, J=85.9 Hz). ³¹P NMR (162 MHz, DMSO-d₆) δ (ppm) 17.32 (d, J=82.5 Hz), 17.08 (d, J=86.2 Hz).

### Step E: ((R)-fluoro(2-(((3S,6S,9S,10aR)-9-(methyl((R)-tetrahydrofuran-3-yl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

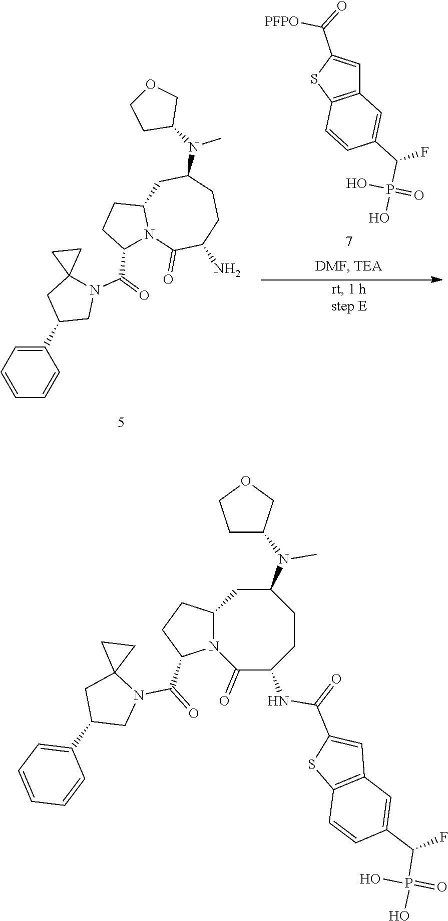

To a solution of (3S,6S,9S,10aR)-6-amino-9-(methyl((R)-tetrahydrofuran-3-yl)amino)-3-((S)-6-phenyl-4-azaspiro[2.4]heptane-4-carbonyl)octahydropyrrolo[1,2-a]azocin-5(1H)-one (5, 20 mg, TFA salt) in DMF (2 mL) was added TEA (10.4 mg, 0.103 mmol, 3 eq.), (R)-(fluoro(2-((perfluorophenoxy)carbonyl)benzo[b]thiophen-5-yl)methyl)phosphonic acid (7, 15.4 mg, 0.034 mmol, 1 eq). The resulting mixture was stirred for 1 h at room temperature, then the crude product was purified by Prep-HPLC to afford ((R)-fluoro(2-(((3S,6S,9S,10aR)-9-(methyl((R)-tetrahydrofuran-3-yl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A19, 16 mg, 0.021 mmol, 61.7%) as a white solid. LCMS (ESI): m/z=753.2 [M+H]⁺. 1H NMR (400 MHz, DMSO-d₆) δ (ppm) 9.77-9.44 (m, 1H), 8.90-8.68 (m, 1H), 8.25 (s, 1H), 8.02 (d, J=8.4 Hz, 1H), 7.97 (s, 1H), 7.53 (d, J=8.5 Hz, 1H), 7.39-7.28 (m, 4H), 7.26-7.20 (m, 1H), 5.81 (dd, J=44.3, 7.9 Hz, 1H), 4.87-4.75 (m, 1H), 4.61-4.45 (m, 2H), 4.18-4.10 (m, 2H), 3.99-3.93 (m, 1H), 3.85-3.80 (m, 1H), 3.74-3.64 (m, 3H), 3.58-3.52 (m, 1H), 2.74-2.62 (m, 3H), 2.48-2.40 (m, 1H), 2.38-2.17 (m, 3H), 2.15-1.90 (m, 8H), 1.89-1.63 (m, 4H), 1.57-1.47 (m, 1H), 0.65-0.41 (m, 2H). ¹⁹F NMR (377 MHz, DMSO-d₆) δ (ppm) −194.82 (d, J=80.3 Hz). ³¹P NMR (162 MHz, DMSO-d₆) δ (ppm) 10.87 (d, J=81.0 Hz).

### Synthesis of ((S)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A31) and propyl (((S)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C51)

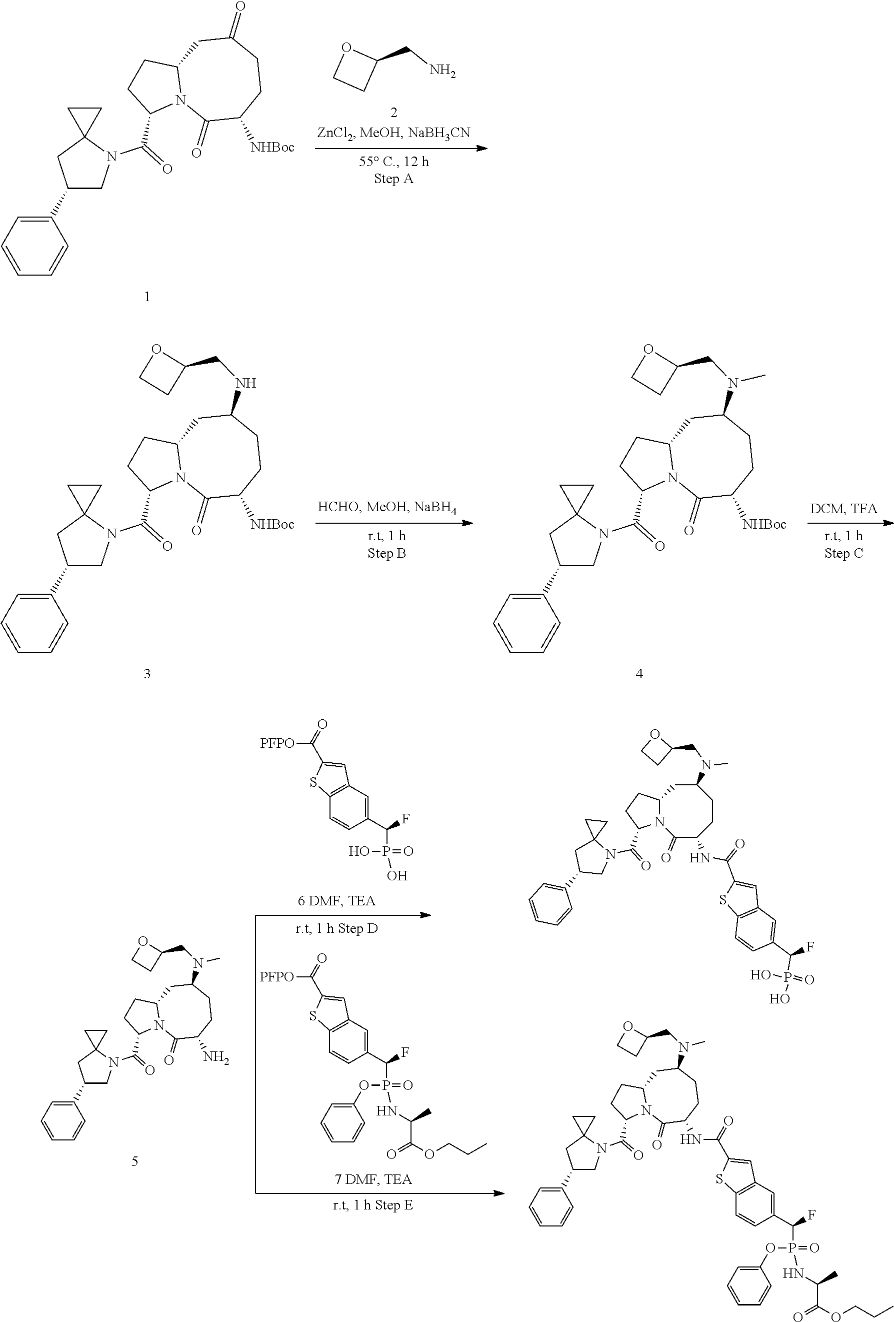

### Step A: tert-butyl ((3S,6S,9S,10aR)-9-((((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamate

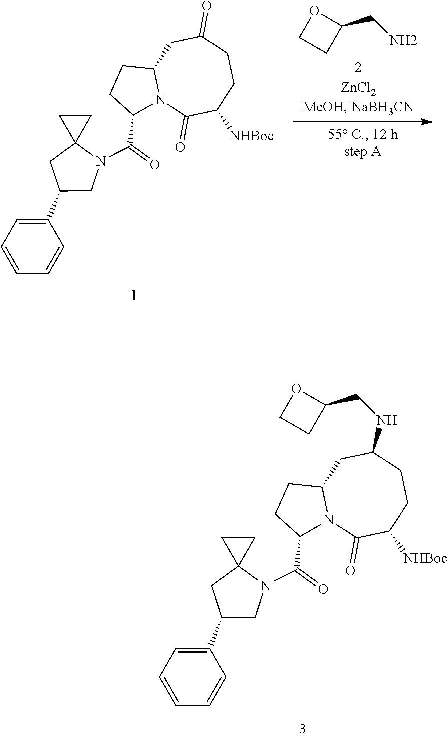 [SMILES: CC(C)(C)OC(=O)N[C@H]1CCC(=O)C[C@H]2CC[C@@H](C(=O)N3C[C@H](c4ccccc4)CC34CC4)N2C1=O ; NC[C@H]1CCO1 ; CC(C)(C)OC(=O)N[C@H]1CC[C@H](NC[C@H]2CCO2)C[C@H]2CC[C@@H](C(=O)N3C[C@H](c4ccccc4)CC34CC4)N2C1=O]

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (1, 150 mg, 302.65 mol, 1 eq.) in MeOH (10 mL) was added (R)-oxetan-2-ylmethanamine (2, 39.55 mg, 453.98 mol, 1.5 eq), ZnCl₂ (412.45 mg, 3.03 mmol, 10 eq.). The solution was stirred for 2 h at 55° C. under the atmosphere of N₂, then NaBH₃CN (190 mg, 3.02 mmol, 10 eq.) was added portion wise every 10 mins (2 eq. (38 mg each time) added 5 times. The mixture was stirred for 12 hr at 55° C., then the reaction mixture was cooled to room temperature, then was used to next step without further workup. LCMS (ESI): m/z=567 [M+H]⁺.

### Step B: tert-butyl ((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamate

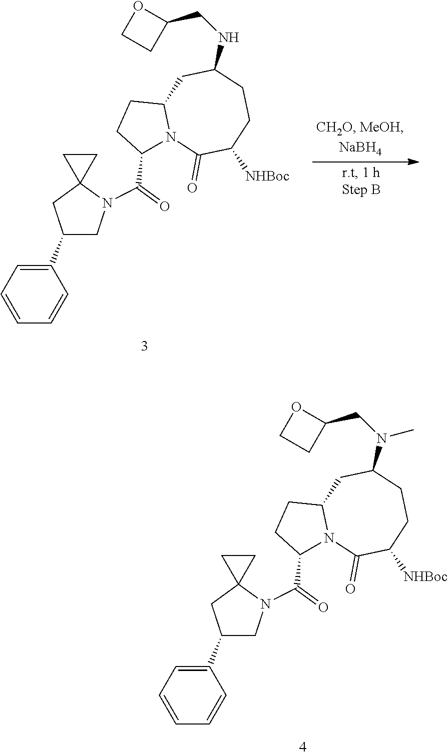 [SMILES: CC(C)(C)OC(=O)N[C@H]1CC[C@H](NC[C@H]2CCO2)C[C@H]2CC[C@@H](C(=O)N3C[C@H](c4ccccc4)CC34CC4)N2C1=O ; CN(C[C@H]1CCO1)[C@H]1CC[C@H](NC(=O)OC(C)(C)C)C(=O)N2[C@H](CC[C@H]2C(=O)N2C[C@H](c3ccccc3)CC23CC3)C1]

To the above solution (Step A) was added 40% formaldehyde (0.5 mL) at room temperature, then NaBH₄ (23 mg, 605.3 mol, 2 eq.), and the mixture was stirred 1 h. LCMS indicated the reaction was completed. Sat. aq. NH₄Cl (20 mL) was added and the mixture was extracted with EtOAc (20 mL×3) and the combined organics was washed with brine, dried (Na₂SO₄), filtered, and concentrated. The crude product was purified by C18 column eluting with (MeCN:H₂O (0.1% ammonium hydroxide)=5%-70%) to afford tert-butyl ((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamate (4, 129 mg, 222.1 mol, 70.7% yield two steps) as a white solid. LCMS (ESI): m/z=581 [M+H]⁺.

### Step C: (3S,6S,9S,10aR)-6-amino-9-(methyl(((R)-oxetan-2-yl)methyl)amino)-3-((S)-6-phenyl-4-azaspiro[2.4]heptane-4-carbonyl)octahydropyrrolo[1,2-a]azocin-5(1H)-one

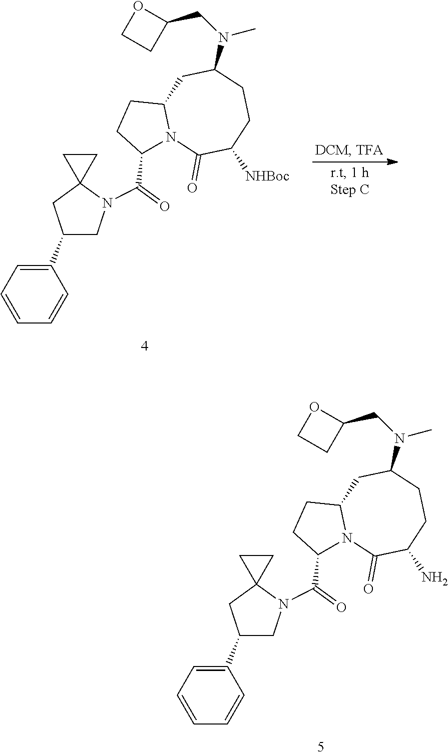 [SMILES: CN(C[C@H]1CCO1)[C@H]1CC[C@H](NC(=O)OC(C)(C)C)C(=O)N2[C@H](CC[C@H]2C(=O)N2C[C@H](c3ccccc3)CC23CC3)C1 ; CN(C[C@H]1CCO1)[C@H]1CC[C@H](N)C(=O)N2[C@H](CC[C@H]2C(=O)N2C[C@H](c3ccccc3)CC23CC3)C1]

To a solution of tert-butyl ((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamate (4, 180 mg, 154.97 mol, 1 eq.) in DCM (3 mL) was added TFA (1 mL). The resulting mixture was stirred for 1 h at room temperature then concentrated to dryness under reduced pressure to afford crude product (3S,6S,9S,10aR)-6-amino-9-(methyl(((R)-oxetan-2-yl)methyl)amino)-3-((S)-6-phenyl-4-azaspiro[2.4]heptane-4-carbonyl)octahydropyrrolo[1,2-a]azocin-5(1H)-one (5, 180 mg, TFA salt) as a yellow oil. LCMS (ESI): m/z=481 [M+H]⁺.

### Step D: ((S)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

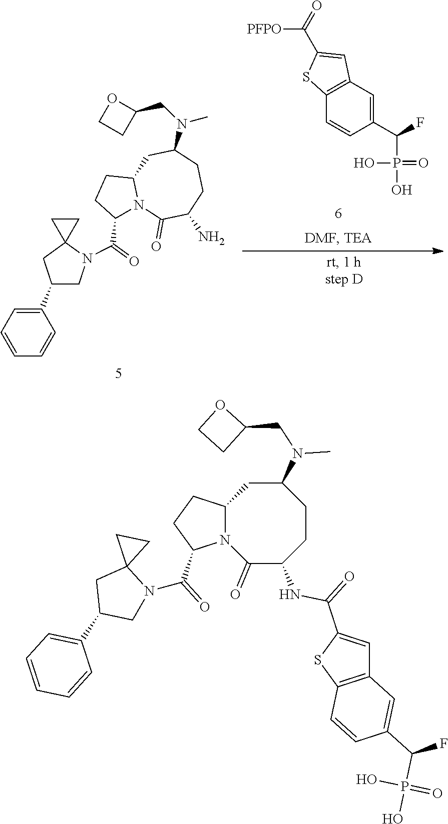

To a solution of (3S,6S,9S,10aR)-6-amino-9-(methyl(((R)-oxetan-2-yl)methyl)amino)-3-((S)-6-phenyl-4-azaspiro[2.4]heptane-4-carbonyl)octahydropyrrolo[1,2-a]azocin-5(1H)-one (5, 45 mg, TFA salt) in DMF (2 mL) were added TEA (23.29 mg. 230.14 mol, 3 eq.) and (S)-(fluoro(2-((perfluorophenoxy)carbonyl)benzo[b]thiophen-5-yl)methyl)phosphonic acid (6, 35 mg, 76.71 mol, 1 eq.), and the resulting mixture was stirred at room temperature for 1 hr, then the crude product was purified by Prep-HPLC to afford ((S)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A31, 18 mg, 23.90 mol) as a white solid. LCMS (ESI): m/z=753.3 (M+H)+. ¹H NMR (400 MHz, DMSO-d₆) δ (ppm) 8.88-8.65 (m, 1H), 8.24 (s, 1H), 8.01 (d, J=8.5 Hz, 1H), 7.97 (s, 1H), 7.52 (d, J=8.5 Hz, 1H), 7.38-7.16 (m, 5H), 5.77 (dd, J=44.6, 7.7 Hz, 1H), 5.26-5.03 (m, 1H), 4.83-4.74 (m, 1H), 4.65-4.42 (m, 4H), 4.16-4.11 (m, 1H), 3.76-3.71 (m, 2H), 3.55-3.51 (m, 2H), 3.28-3.20 (m, 1H), 2.83-2.61 (m, 4H), 2.48-2.32 (m, 2H), 2.29-2.18 (m, 1H), 2.15-1.66 (m, 11H), 1.61-1.53 (m, 1H), 0.60-0.44 (m, 2H). ¹⁹F NMR (376 MHz, DMSO-d₆) δ (ppm) −194.52 (d, J=80.3 Hz). ³¹P NMR (162 MHz, DMSO-d₆) δ (ppm) 10.73 (d, J=79.4 Hz).

### Step E: propyl (((S)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

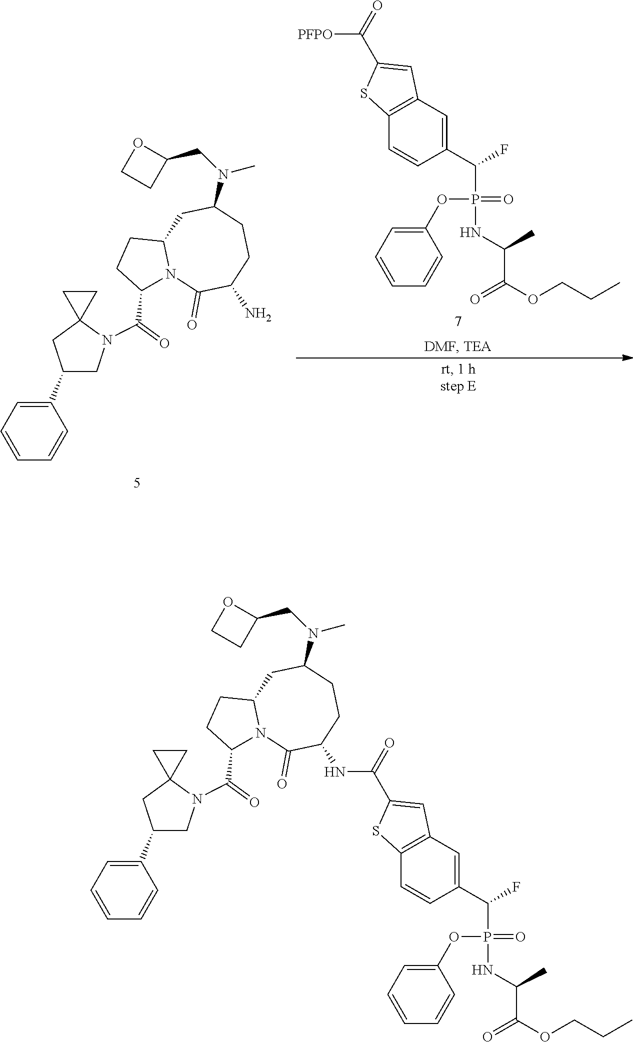

To a solution of (3S,6S,9S,10aR)-6-amino-9-(methyl(((R)-oxetan-2-yl)methyl)amino)-3-((S)-6-phenyl-4-azaspiro[2.4]heptane-4-carbonyl)octahydropyrrolo[1,2-a]azocin-5(1H)-one (5, 45 mg, TFA salt) in DMF (2 mL) were added TEA (23.51 mg. 232.37 mol, 3 eq.) and perfluorophenyl 5-((1S)-fluoro((((S)-1-oxo-1-propoxypropan-2-yl)amino)(phenoxy)phosphoryl)methyl)benzo[b]thiophene-2-carboxylate (7, 50 mg, 77.46 mol, 1 eq.), and the resulting mixture was stirred at room temperature for 1 hr. The crude product was purified by Prep-HPLC to afford propyl (((S)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C51, 25 mg, 26.54 mol, 34.3%) as a white solid. LCMS (ESI): m/z=942.5 (M+H)+. 1H NMR (400 MHz, DMSO-d₆) δ (ppm) 8.80-8.68 (m, 1H), 8.30-8.22 (m, 1H), 8.12-7.99 (m, 2H), 7.65-7.55 (m, 1H), 7.40-7.26 (m, 6H), 7.23-7.10 (m, 4H), 6.30-6.07 (m, 2H), 4.85-4.67 (m, 2H), 4.53-4.42 (m, 2H), 4.41-4.34 (m, 1H), 4.33-4.25 (m, 1H), 4.16-4.06 (m, 1H), 3.96-3.63 (m, 4H), 3.58-3.47 (m, 1H), 2.96-2.84 (m, 1H), 2.73-2.52 (m, 3H), 2.41-2.21 (m, 3H), 2.20-2.08 (m, 4H), 2.04-1.92 (m, 3H), 1.90-1.61 (m, 6H), 1.59-1.40 (m, 4H), 1.19-0.92 (m, 3H), 0.81 (t, J=7.4 Hz, 3H), 0.55-0.39 (m, 2H). ¹⁹F NMR (377 MHz, DMSO-d₆) δ (ppm) −194.14 (d, J=82.5 Hz), −196.07 (d, J=86.8 Hz). ³¹P NMR (162 MHz, DMSO-d₆) δ (ppm) 17.32 (d, J=86.6 Hz), 16.94 (d, J=82.5 Hz).

### Synthesis of ((R)-fluoro(2-(((3S,6S,9S,10aR)-9-(methyl(oxetan-3-yl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A13) and propyl ((R)-((S)-fluoro(2-(((3S,6S,9S,10aR)-9-(methyl(oxetan-3-yl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C30)

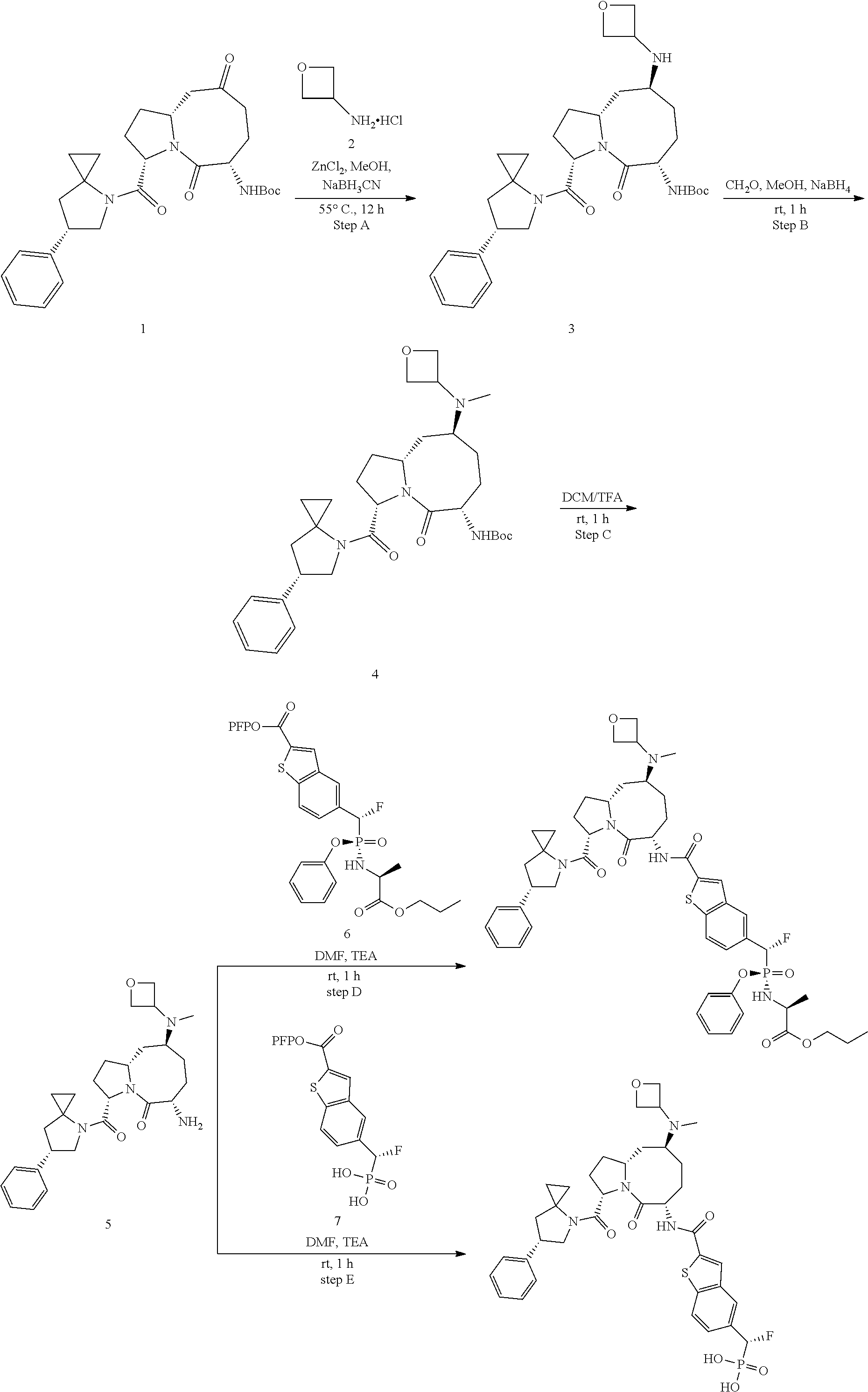

### Step A: tert-butyl ((3S,6S,9S,10aR)-9-(oxetan-3-ylamino)-5-oxo-3-((R)-6-phenyl-4-azaspiro[2.4]heptane-4-carbonyl)decahydropyrrolo[1,2-a]azocin-6-yl)carbamate

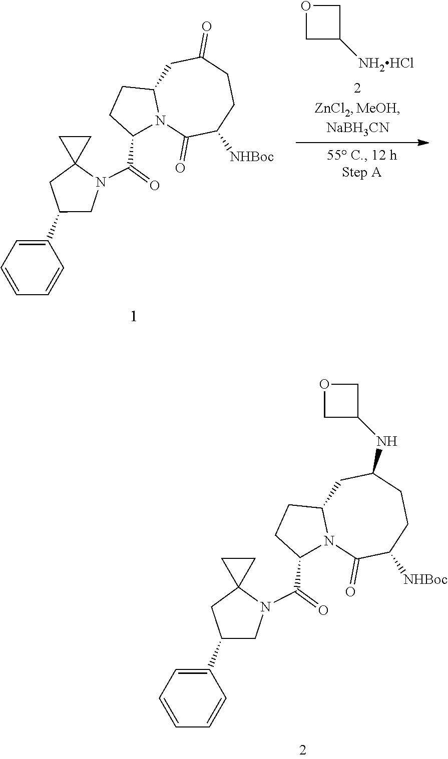

To a solution of tert-butyl ((3S,6S,10aR)-5,9-dioxo-3-((R)-6-phenyl-4-azaspiro[2.4]heptane-4-carbonyl)decahydropyrrolo[1,2-a]azocin-6-yl)carbamate (1, 2 g, 4.04 mmol, 1 eq.) in MeOH (40 mL) was added oxetan-3-amine hydrochloride (2, 663.13 mg, 6.05 mmol, 1.5 eq), ZnCl₂ (5.50 g, 40.35 mmol, 10 eq.). The resulting mixture was stirred for 2 h at 55° C. under the atmosphere of N₂, then NaBH₃CN (2.54 g, 40.4 mmol, 10 eq) was added portion wise every 30 mins (2 eq. (508 mg each time) added 5 times). The solution was stirred for 12 h at 55° C., then the reaction mixture was cooled to room temperature, then was used to next step without further workup. LCMS (ESI): m/z=553 [M+H]⁺.

### Step B: tert-butyl ((3S,6S,9S,10aR)-9-(methyl(oxetan-3-yl)amino)-5-oxo-3-((R)-6-phenyl-4-azaspiro[2.4]heptane-4-carbonyl)decahydropyrrolo[1,2-a]azocin-6-yl)carbamate

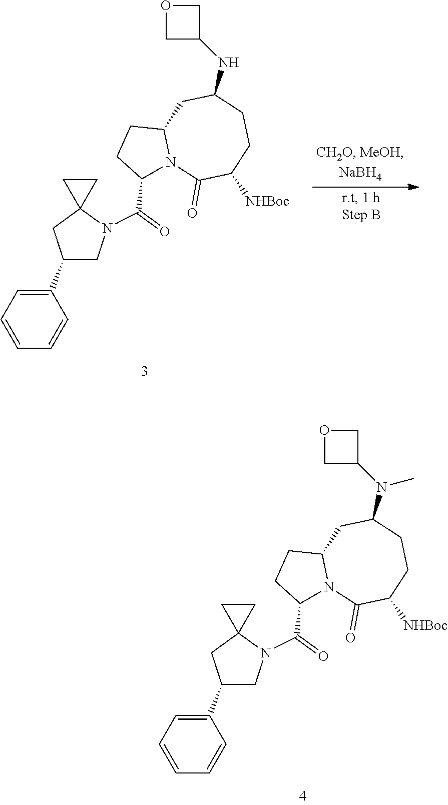 [SMILES: CC(C)(C)OC(=O)N[C@H]1CC[C@H](NC2COC2)C[C@H]2CC[C@@H](C(=O)N3C[C@H](c4ccccc4)CC34CC4)N2C1=O ; CN(C1COC1)[C@H]1CC[C@H](NC(=O)OC(C)(C)C)C(=O)N2[C@H](CC[C@H]2C(=O)N2C[C@H](c3ccccc3)CC23CC3)C1]

To the above solution (Step A) was added 40% formaldehyde (3 mL) at room temperature, and the solution was stirred for 30 min, then NaBH₄ (764 mg, 20.2 mmol, 5 eq.) in several portions, the mixture was stirred for additional 30 mins, the reaction was quenched by NH₄Cl (sat. aq), diluted with H₂O (150 mL), extracted with EtOAc (60 mL×3), the organic solution was dried over Na₂SO₄, then concentrated under vacuum, filtered, and the crude product was purified by C18 column (MeCN:H₂O (0.1% ammonium hydroxide)=5%-70%) to afford tert-butyl ((3S,6S,9S,10aR)-9-(methyl(oxetan-3-yl)amino)-5-oxo-3-((R)-6-phenyl-4-azaspiro[2.4]heptane-4-carbonyl)decahydropyrrolo[1,2-a]azocin-6-yl)carbamate (4, 1.8 g, 3.17 mmol, 78.6% yield two steps) as a white solid. LCMS (ESI): m/z=567 [M+H]⁺.

### Step C: (3S,6S,9S,10aR)-6-amino-9-(methyl(oxetan-3-yl)amino)-3-((R)-6-phenyl-4-azaspiro[2.4]heptane-4-carbonyl)octahydropyrrolo[1,2-a]azocin-5(1H)-one

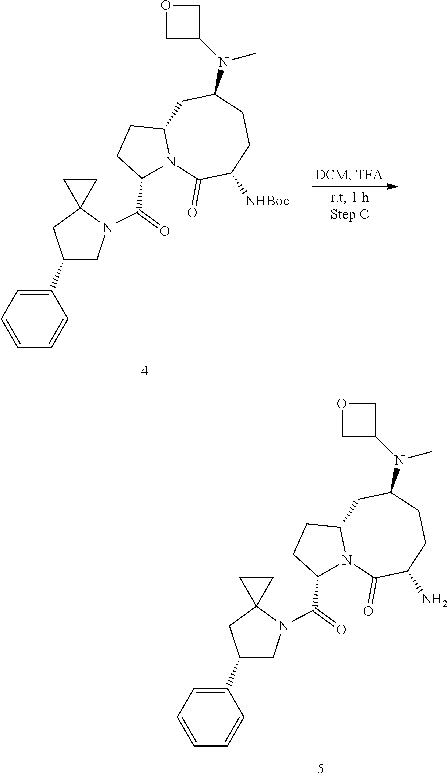 [SMILES: CN(C1COC1)[C@H]1CC[C@H](NC(=O)OC(C)(C)C)C(=O)N2[C@H](CC[C@H]2C(=O)N2C[C@H](c3ccccc3)CC23CC3)C1 ; CN(C1COC1)[C@H]1CC[C@H](N)C(=O)N2[C@H](CC[C@H]2C(=O)N2C[C@H](c3ccccc3)CC23CC3)C1]

To a solution of tert-butyl ((3S,6S,9S,10aR)-9-(methyl(oxetan-3-yl)amino)-5-oxo-3-((R)-6-phenyl-4-azaspiro[2.4]heptane-4-carbonyl)decahydropyrrolo[1,2-a]azocin-6-yl)carbamate (4, 1.8 g, 3.17 mmol, 1 eq.) in DCM (18 mL) was added TFA (6 mL). The resulting mixture was stirred for 1 hr at room temperature. The resulting mixture was concentrated to dryness under reduced pressure to afford crude product (3S,6S,9S,10aR)-6-amino-9-(methyl(oxetan-3-yl)amino)-3-((R)-6-phenyl-4-azaspiro[2.4]heptane-4-carbonyl)octahydropyrrolo[1,2-a]azocin-5(1H)-one (5, 1.8 g, TFA salt) as a yellow oil. LCMS (ESI): m/z=467 [M+H]⁺.

### Step D: propyl ((R)-((S)-fluoro(2-(((3S,6S,9S,10aR)-9-(methyl(oxetan-3-yl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

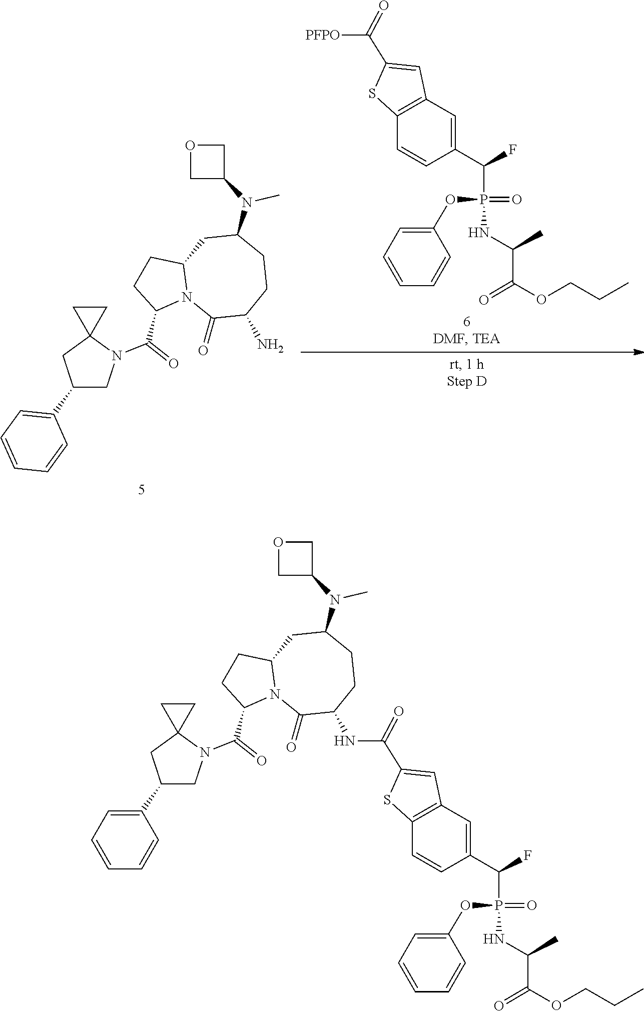

To a solution of (3S,6S,9S,10aR)-6-amino-9-(methyl(oxetan-3-yl)amino)-3-((R)-6-phenyl-4-azaspiro[2.4]heptane-4-carbonyl)octahydropyrrolo[1,2-a]azocin-5(1H)-one (5, 50 mg, TFA salt) in DMF (2 mL) were added TEA (25.75 mg. 0.255 mmol, 3 eq.) and perfluorophenyl 5-((S)-fluoro((R)-(((S)-1-oxo-1-propoxypropan-2-yl)amino)(phenoxy)phosphoryl)methyl)benzo[b]thiophene-2-carboxylate (6, 55 mg, 0.085 mmol, 1 eq.), and the resulting mixture was stirred at room temperature for 1 hr. The crude product was purified by Prep-HPLC to afford propyl ((R)-((S)-fluoro(2-(((3S,6S,9S,10aR)-9-(methyl(oxetan-3-yl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C30, 41 mg, 0.044 mmol, 51.8%) as a white solid. LCMS (ESI): m/z=928.5 [M+H]⁺. 1H NMR (400 MHz, DMSO-d₆) δ (ppm) 8.78 (d, J=7.6 Hz, 1H), 8.26 (s, 1H), 8.08-8.02 (m, 2H), 7.59 (d, J=8.5 Hz, 1H), 7.38-7.27 (m, 6H), 7.23-7.09 (m, 4H), 6.30-6.14 (m, 2H), 4.76-4.65 (m, 1H), 4.58-4.50 (m, 2H), 4.49-4.39 (m, 2H), 4.37-4.33 (m, 1H), 4.32-4.24 (m, 1H), 4.15-4.07 (m, 1H), 3.95-3.81 (m, 3H), 3.79-3.67 (m, 2H), 3.60-3.47 (m, 1H), 2.88-2.78 (m, 1H), 2.42-2.31 (m, 1H), 2.28-2.18 (m, 1H), 2.16-2.07 (m, 1H), 2.04-1.90 (m, 6H), 1.84-1.55 (m, 7H), 1.54-1.40 (m, 3H), 1.17 (d, J=7.1 Hz, 3H), 0.81 (t, J=7.4 Hz, 3H), 0.60-0.38 (m, 2H). ¹⁹F NMR (376 MHz, DMSO-d₆) δ (ppm) −196.08 (d, J=86.6 Hz). ³¹P NMR (162 MHz, DMSO-d₆) δ (ppm) 17.32 (d, J=87.4 Hz).

### Step E: ((R)-fluoro(2-(((3S,6S,9S,10aR)-9-(methyl(oxetan-3-yl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

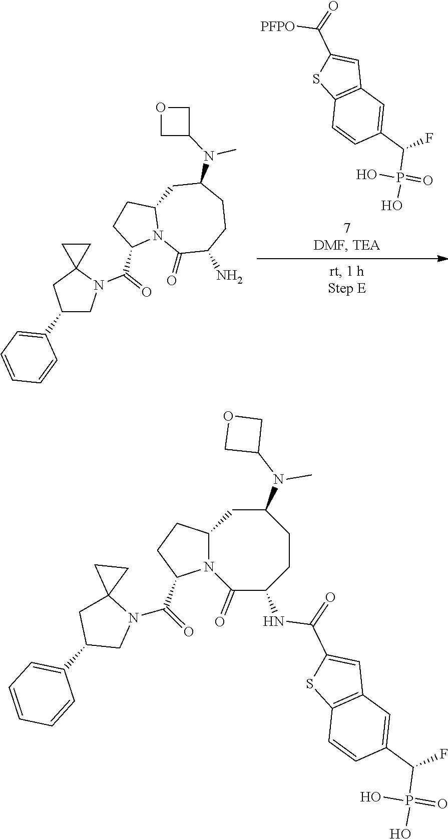

To a solution of (3S,6S,9S,10aR)-6-amino-9-(methyl(oxetan-3-yl)amino)-3-((R)-6-phenyl-4-azaspiro[12.4]heptane-4-carbonyl)octahydropyrrolo[1,2-a]azocin-5(1H)-one (5, 50 mg, TFA salt) in DMF (2 mL) were added TEA (25.75 mg. 0.255 mmol, 3 eq.) and (R)-(fluoro(2-((perfluorophenoxy)carbonyl)benzo[b]thiophen-5-yl)methyl)phosphonic acid (7, 40 mg, 0.088 mmol, 1 eq.), and the resulting mixture was stirred at room temperature for 1 h. The crude product was purified by Prep-HPLC to afford ((R)-fluoro(2-(((3S,6S,9S,10aR)-9-(methyl(oxetan-3-yl)amino)-5-oxo-3-((R)-6-phenyl-4-azaspiro[2.4]heptane-4-carbonyl)decahydropyrrolo[[1,2-a]azocin-6-yl)carbamoyl)benzo(b]thiophen-5-yl)methyl)phosphonic acid (Compound A13, 36 mg, 0.048 mmol, 54.5%) as a white solid. LCMS (ESI): m/z=739.6 [M+H]⁺. ¹H NMR (400 MHz, DMSO-d₆) δ (ppm) 10.85-10.19 (m, 1H), 8.87-8.68 (m, 1H), 8.24 (s, 1H), 8.03 (d, J=8.5 Hz, 1H), 7.97 (s, 1H), 7.53 (d, J=8.6 Hz, 1H), 7.38-7.30 (m, 4H), 7.25-7.21 (m, 1H), 5.83 (dd, J=44.4, 8.0 Hz, 1H), 4.92-4.50 (m, 8H), 4.18-4.09 (m, 1H), 3.74-3.68 (m, 1H), 3.60-3.52 (m, 2H), 2.60 (s, 3H), 2.48-2.41 (m, 1H), 2.28-2.21 (m, 1H), 2.16-1.70 (m, 11H), 1.57-1.51 (m, 1H), 0.61-0.48 (m, 2H). ¹⁹F NMR (377 MHz, DMSO-d₆) δ (ppm) −194.91 (d, J=80.8 Hz). ³¹P NMR (162 MHz, DMSO-d₆) δ (ppm) 10.92 (d, J=80.9 Hz).

### Synthesis of ((R)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A17) and propyl (((1R)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)-2,3-dihydrobenzo[b]thiophen-5-yl)methyl)(phenoxy)phosphoryl)-L-alaninate (Compound C20)

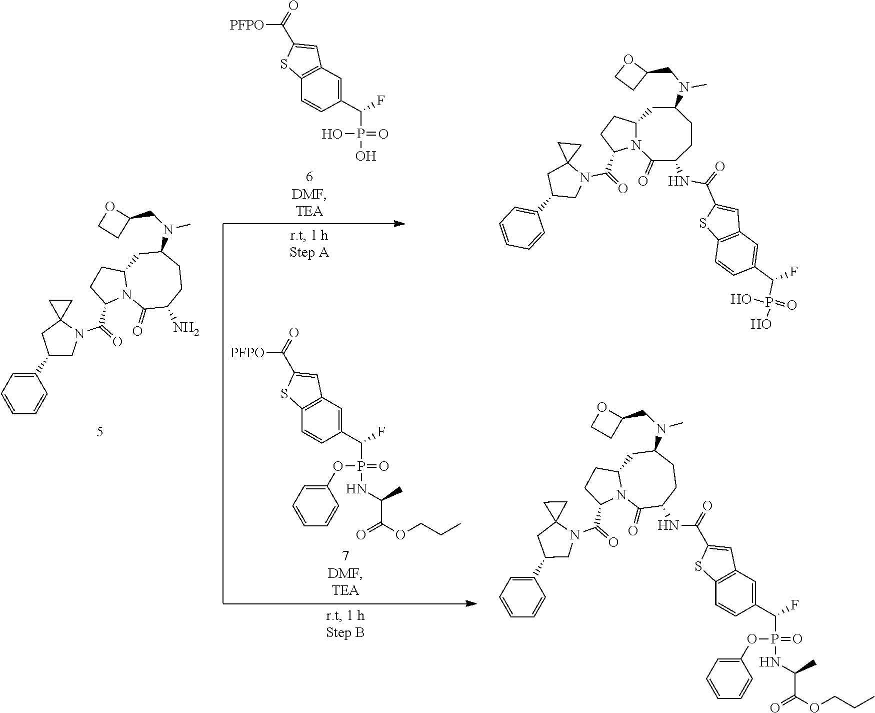

### Step A: ((R)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

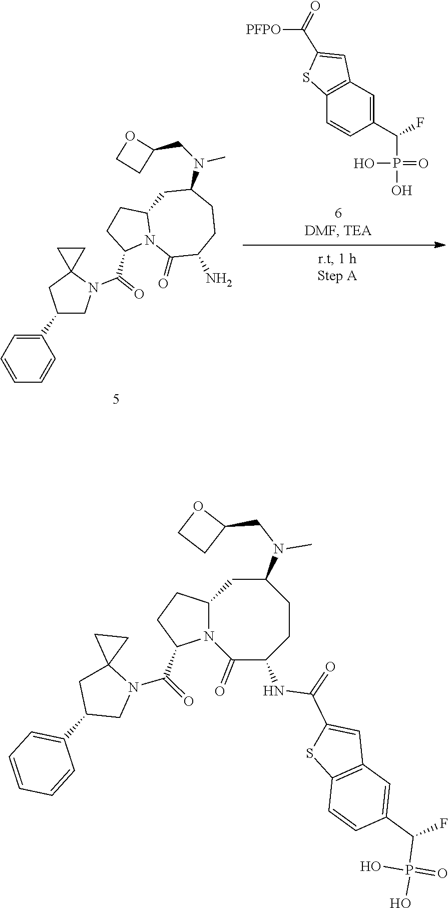

To a solution of (3S,6S,9S,10aR)-6-amino-9-(methyl(((R)-oxetan-2-yl)methyl)amino)-3-((S)-6-phenyl-4-azaspiro[2.4]heptane-4-carbonyl)octahydropyrrolo[1,2-a]azocin-5(1H)-one (5, 45 mg, TFA salt) in DMF (2 mL) were added TEA (23.29 mg. 230.14 mol, 3 eq.) and (R)-(fluoro(2-((perfluorophenoxy)carbonyl)benzo[b]thiophen-5-yl)methyl)phosphonic acid (6, 35 mg, 76.71 mol, 1 eq.), and the resulting mixture was stirred at room temperature for 1 h. The crude product was purified by Prep-HPLC to afford ((R)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A17, 11.7 mg, 15.54 mol, 20.2%) as a white solid. LCMS (ESI): m/z=753.2 [M+H]⁺. ¹H NMR (400 MHz, DMSO-d₆) δ (ppm) 9.44 (d, J=48.2 Hz, 1H), 8.77 (d, J=15.4 Hz, 1H), 8.25 (s, 1H), 8.04-7.89 (m, 2H), 7.52 (d, J=8.5 Hz, 1H), 7.39-7.20 (m, 5H), 5.78 (dd, J=44.4, 7.9 Hz, 1H), 5.14 (d, J=40.9 Hz, 1H), 4.84-4.73 (m, 1H), 4.58-4.52 (m, 2H), 4.14 (s, 1H), 3.73 (d, J=12.7 Hz, 2H), 3.57-3.50 (m, 2H), 3.35-3.26 (m, 1H), 3.20 (s, 1H), 2.79-2.63 (m, 4H), 2.48-2.15 (m, 4H), 2.13-1.71 (m, 11H), 1.63-1.55 (m, 1H), 0.53 (s, 2H). (TFA salt). ¹⁹F NMR (377 MHz, DMSO-d₆) δ (ppm) −36.21-−36.91 (m). ³¹P NMR (162 MHz, DMSO-d₆) δ (ppm) 10.78 (d, J=80.1 Hz).

### Step B: propyl (((R)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

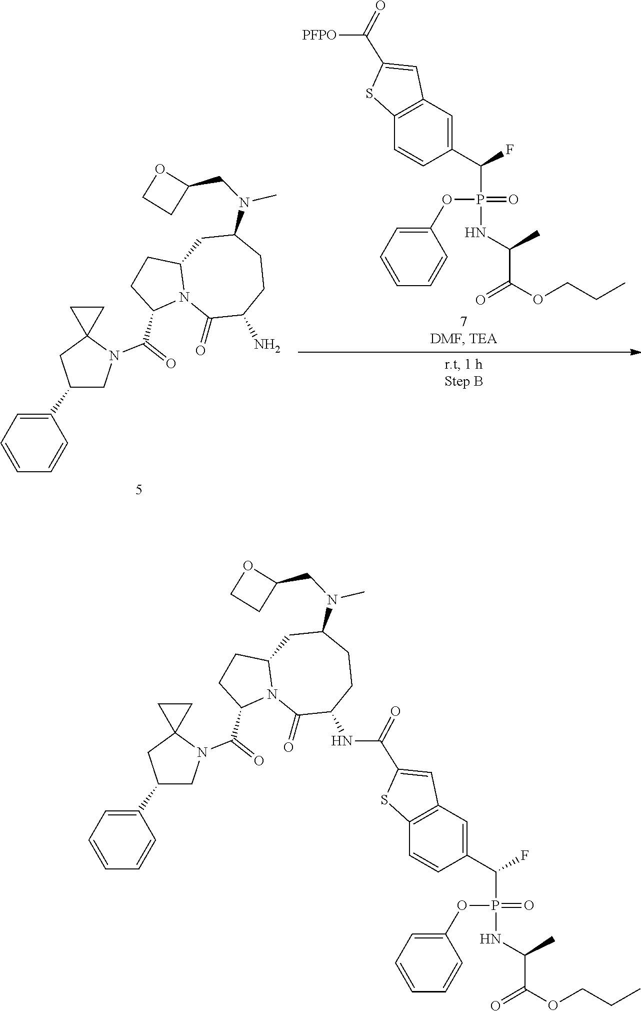

Procedure for 5 is described in the synthesis of Compound A31 and Compound C51. To a solution of (3S,6S,9S,10aR)-6-amino-9-(methyl(((R)-oxetan-2-yl)methyl)amino)-3-((S)-6-phenyl-4-azaspiro[12.4]heptane-4-carbonyl)octahydropyrrolo[1,2-a]azocin-5(1H)-one (5, 45 mg, TFA salt) in DMF (2 mL) were added TEA (23.51 mg. 232.37 mol, 3 eq.) and perfluorophenyl 5-((1R)-fluoro((((S)-1-oxo-1-propoxypropan-2-yl)amino)(phenoxy)phosphoryl)methyl)benzo[b]thiophene-2-carboxylate (7, 50 mg, 77.46 μmol, 1 eq.), and the resulting mixture was stirred at room temperature for 1 hr. LCMS indicated the complete consumption of the starting material. The crude product was purified by Prep-HPLC to afford propyl (((R)-fluoro(2-(((3S,6S,9S,10aR)-9-(methyl(((R)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C20, 26.6 mg, 28.23 mol, 36.4%) as a white solid. LCMS (ESI): m/z 943.0 [M+H]⁺. ¹H NMR (400 MHz, DMSO-do) δ (ppm) 8.83-8.69 (m, 1H), 8.27 (s, 1H), 8.09-8.02 (m, 2H), 7.65-7.58 (m, 1H), 7.40-7.26 (m, 6H), 7.24-7.13 (m, 4H), 6.29-5.99 (m, 2H), 4.86-4.67 (m, 2H), 4.54-4.41 (m, 2H), 4.40-4.34 (m, 1H), 4.33-4.25 (m, 1H), 4.12 (t, J=8.5 Hz, 1H), 4.00-3.88 (m, 1H), 3.87-3.68 (m, 3H), 3.59-3.48 (m, 1H), 2.93-2.87 (m, 1H), 2.70-2.51 (m, 3H), 2.40-2.19 (m, 3H), 2.16 (s, 3H), 2.14-2.08 (m, 1H), 2.04-1.92 (m, 3H), 1.90-1.62 (m, 6H), 1.61-1.36 (m, 4H), 1.18-1.07 (m, 3H), 0.86-0.73 (m, 3H), 0.54-0.40 (m, 2H). ¹⁹F NMR (377 MHz, DMSO-d₆) δ (ppm) −194.27 (d, J=82.7 Hz), −196.29 (d, J=86.0 Hz). ³¹P NMR (162 MHz, DMSO-d₆) δ (ppm) 17.32 (d, J=82.8 Hz), 17.07 (d, J=86.1 Hz).

### Synthesis of ((R)-fluoro(2-(((3S,6S,9S,10aR)-9-(methyl(((S)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A15) and propyl (((R)-fluoro(2-(((3S,6S,9S,10aR)-9-(methyl(((S)-oxetan-2-yl)methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C18)

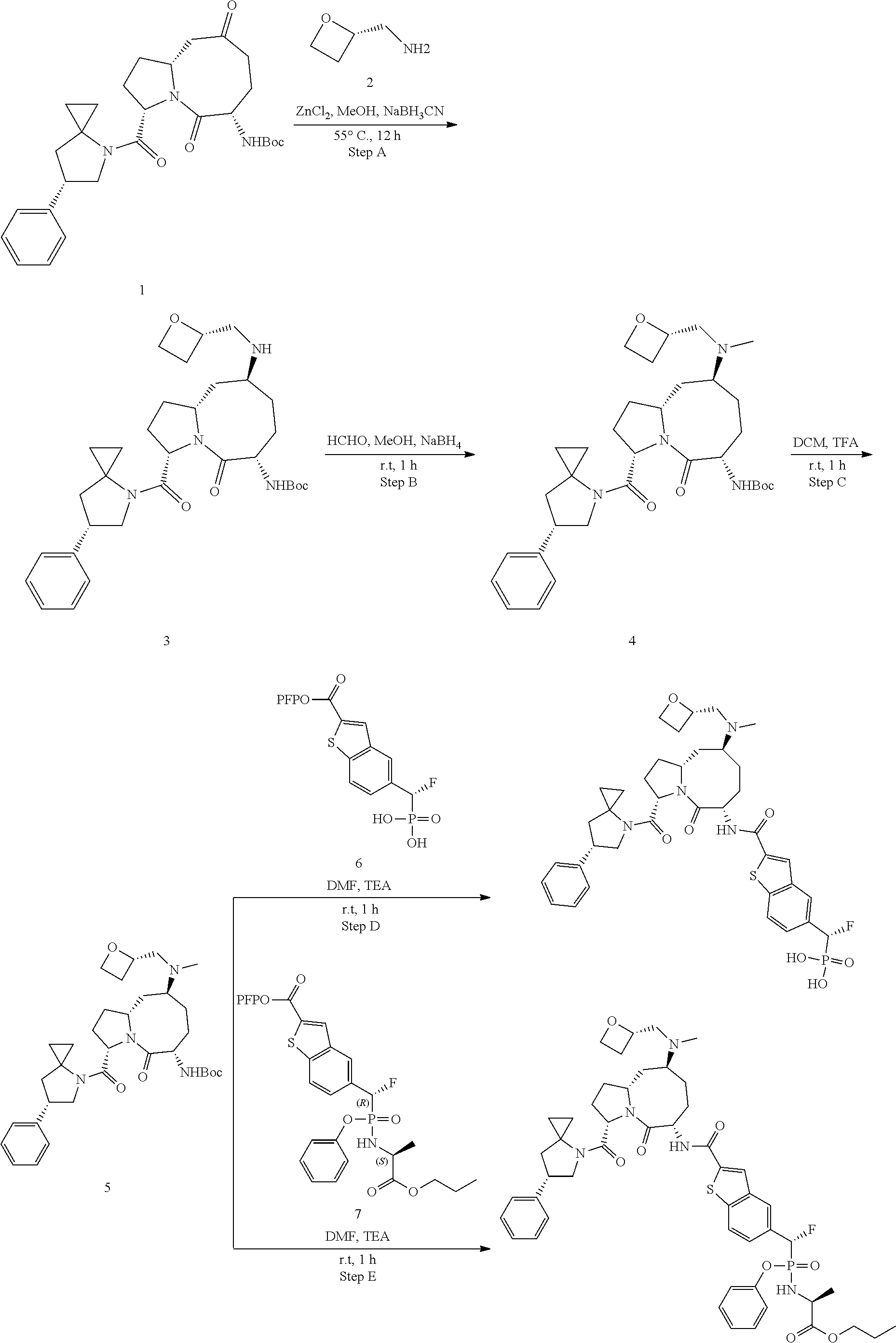

### Step A: tert-butyl ((3S,6S,9S,10aR)-9-((((S)-oxetan-2-yl)methyl)amino)-5-oxo-3-((R)-6-phenyl-4-azaspiro[2.4]heptane-4-carbonyl)decahydropyrrolo[1,2-a]azocin-6-yl)carbamate

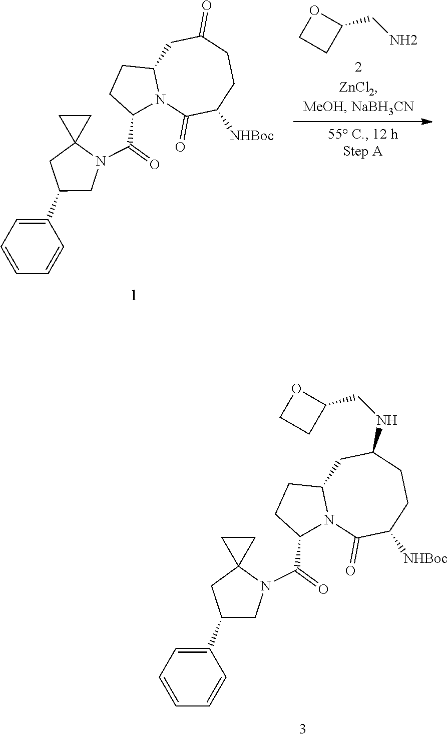 [SMILES: CC(C)(C)OC(=O)N[C@H]1CCC(=O)C[C@H]2CC[C@@H](C(=O)N3C[C@H](c4ccccc4)CC34CC4)N2C1=O ; NC[C@@H]1CCO1 ; CC(C)(C)OC(=O)N[C@H]1CC[C@H](NC[C@@H]2CCO2)C[C@H]2CC[C@@H](C(=O)N3C[C@H](c4ccccc4)CC34CC4)N2C1=O]

To a solution of tert-butyl ((3S,6S,10aR)-5,9-dioxo-3-((R)-6-phenyl-4-azaspiro[2.4]heptane-4-carbonyl)decahydropyrrolo[1,2-a]azocin-6-yl)carbamate (1, 150 mg, 302.65 mol, 1 eq.) in MeOH (5 mL) was added (S)-oxetan-2-ylmethanamine (2, 39.55 mg, 453.98 mol, 1.5 eq), ZnCl₂ (412.45 mg, 3.03 mmol, 10 eq.). The resulting mixture was stirred for 2 h at 55° C. under the atmosphere of N₂, then NaBH₃CN (190 mg, 3.03 mmol, 2 eq) was added portion wise every 10 mins (2 eq. (38 mg each time) added 5 times). The solution was stirred for 12 h at 55° C., then the reaction mixture was cooled to room temperature, then was used to next step without further workup. LCMS (ESI): m/z=567 [M+H]⁺.

### Step B: tert-butyl ((3S,6S,9S,10aR)-9-(methyl(((S)-oxetan-2-yl)methyl)amino)-5-oxo-3-((R)-6-phenyl-4-azaspiro[2.4]heptane-4-carbonyl)decahydropyrrolo[1,2-a]azocin-6-yl)carbamate

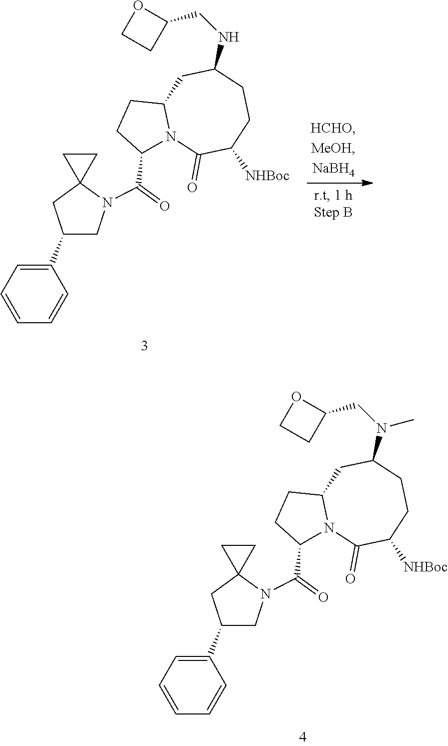 [SMILES: CC(C)(C)OC(=O)N[C@H]1CC[C@H](NC[C@@H]2CCO2)C[C@H]2CC[C@@H](C(=O)N3C[C@H](c4ccccc4)CC34CC4)N2C1=O ; CN(C[C@@H]1CCO1)[C@H]1CC[C@H](NC(=O)OC(C)(C)C)C(=O)N2[C@H](CC[C@H]2C(=O)N2C[C@H](c3ccccc3)CC23CC3)C1]

To the above solution (Step A) was added 40% formaldehyde (0.5 mL) at room temperature, and the solution was stirred for 30 min, then NaBH₄ (57.25 mg, 1.51 mmol, 5 eq) was added in one portion, the mixture was stirred for another 30 mins, then the reaction was quenched by NH₄Cl (sat. aq), diluted with H₂O (20 mL), then extracted with EtOAc (10 mL×3), the organic solution was dried over Na₂SO₄, filtered, then concentrated under vacuum. The crude product was purified by C18 column (MeCN:H₂O (0.1% ammonium hydroxide)=5%-70%) to afford tert-butyl ((3S,6S,9S,10aR)-9-(methyl(((S)-oxetan-2-yl)methyl)amino)-5-oxo-3-((R)-6-phenyl-4-azaspiro[2.4]heptane-4-carbonyl)decahydropyrrolo[1,2-a]azocin-6-yl)carbamate (4, 120 mg, 206.6 mol, 68.2% yield two steps) as a white solid. LCMS (ESI): m/z=581 [M+H]⁺.

### Step C: (3S,6S,9S,10aR)-6-amino-9-(methyl(((S)-oxetan-2-yl)methyl)amino)-3-((R)-6-phenyl-4-azaspiro[2.4]heptane-4-carbonyl)octahydropyrrolo[1,2-a]azocin-5(1H)-one

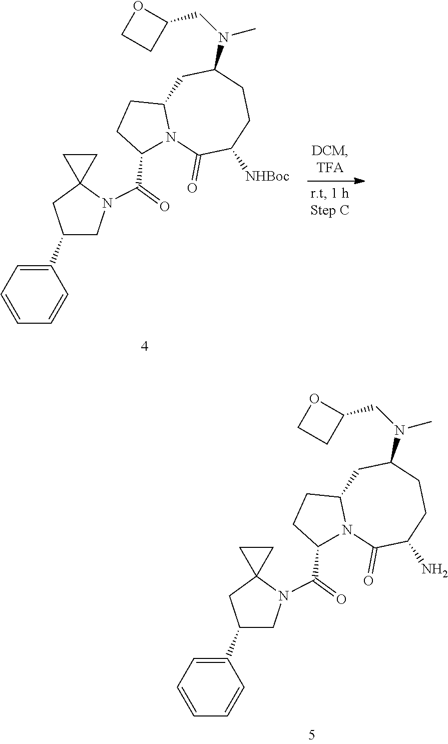 [SMILES: CN(C[C@@H]1CCO1)[C@H]1CC[C@H](NC(=O)OC(C)(C)C)C(=O)N2[C@H](CC[C@H]2C(=O)N2C[C@H](c3ccccc3)CC23CC3)C1 ; CN(C[C@@H]1CCO1)[C@H]1CC[C@H](N)C(=O)N2[C@H](CC[C@H]2C(=O)N2C[C@H](c3ccccc3)CC23CC3)C1]

To a solution of tert-butyl ((3S,6S,9S,10aR)-9-(methyl(((S)-oxetan-2-yl)methyl)amino)-5-oxo-3-((R)-6-phenyl-4-azaspiro[2.4]heptane-4-carbonyl)decahydropyrrolo[1,2-a]azocin-6-yl)carbamate (4, 90 mg, 154.97 mol, 1 eq.) in DCM (3 mL) was added TFA (1 mL). The resulting mixture was stirred for 1 h at room temperature. The resulting mixture was concentrated to dryness under reduced pressure to afford crude product (3S,6S,9S,10aR)-6-amino-9-(methyl(((S)-oxetan-2-yl)methyl)amino)-3-((R)-6-phenyl-4-azaspiro[2.4]heptane-4-carbonyl)octahydropyrrolo[1,2-a]azocin-5(1H)-one (5, 90 mg, TFA salt) as a yellow oil. LCMS (ESI): m/z=481 [M+H]⁺.

### Step D: ((R)-fluoro(2-(((3S,6S,9S,10aR)-9-(methyl(((S)-oxetan-2-yl)methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

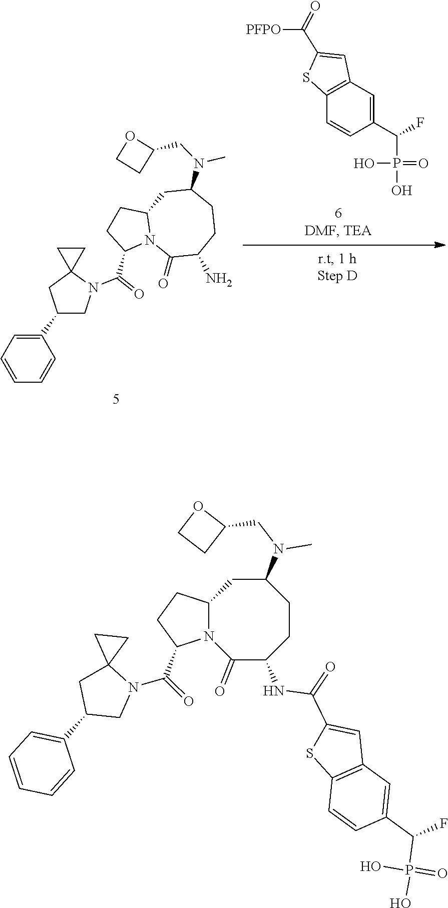

To a solution of (3S,6S,9S,10aR)-6-amino-9-(methyl(((S)-oxetan-2-yl)methyl)amino)-3-((R)-6-phenyl-4-azaspiro[2.4]heptane-4-carbonyl)octahydropyrrolo[1,2-a]azocin-5(1H)-one (5, 45 mg, TFA salt) in DMF (2 mL) were added TEA (23.29 mg. 230.14 mol, 3 eq.) and (R)-(fluoro(2-((perfluorophenoxy)carbonyl)benzo[b]thiophen-5-yl)methyl)phosphonic acid (6, 35 mg, 76.71 mol, 1 eq.), and the resulting mixture was stirred at room temperature for 1 h. The crude product was purified by Prep-HPLC to afford ((R)-fluoro(2-(((3S,6S,9S,10aR)-9-(methyl(((S)-oxetan-2-yl)methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A15, 22.8 mg, 30.28 mol, 39.4%) as a white solid. LCMS (ESI): m/z=753.3 [M+H]⁺. ¹H NMR (400 MHz, DMSO-d₆) δ (ppm) 9.61-9.30 (m, 1H), 8.86-8.69 (m, 1H), 8.29-8.22 (m, 1H), 8.06-7.93 (m, 2H), 7.53 (d, J=8.5 Hz, 1H), 7.40-7.29 (m, 4H), 7.23 (t, J=7.0 Hz, 1H), 5.81 (dd, J=44.4, 8.0 Hz, 1H), 5.21-5.06 (m, 1H), 4.84-4.75 (m, 1H), 4.62-4.51 (m, 3H), 4.15-4.09 (m, 1H), 3.78-3.66 (m, 2H), 3.60-3.48 (m, 2H), 3.38-3.16 (m, 1H), 2.83-2.64 (m, 4H), 2.49-1.70 (m, 15H), 1.63-1.53 (m, 1H), 0.57-0.43 (m, 2H). (TFA salt). ¹⁹F NMR (376 MHz, DMSO-d₆) δ (ppm) −194.80 (d, J=80.8 Hz). ³¹P NMR (162 MHz, DMSO-d₆) δ (ppm) 10.86 (d, J=80.8 Hz).

### Step E: propyl (((R)-fluoro(2-(((3S,6S,9S,10aR)-9-(methyl(((S)-oxetan-2-yl)methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

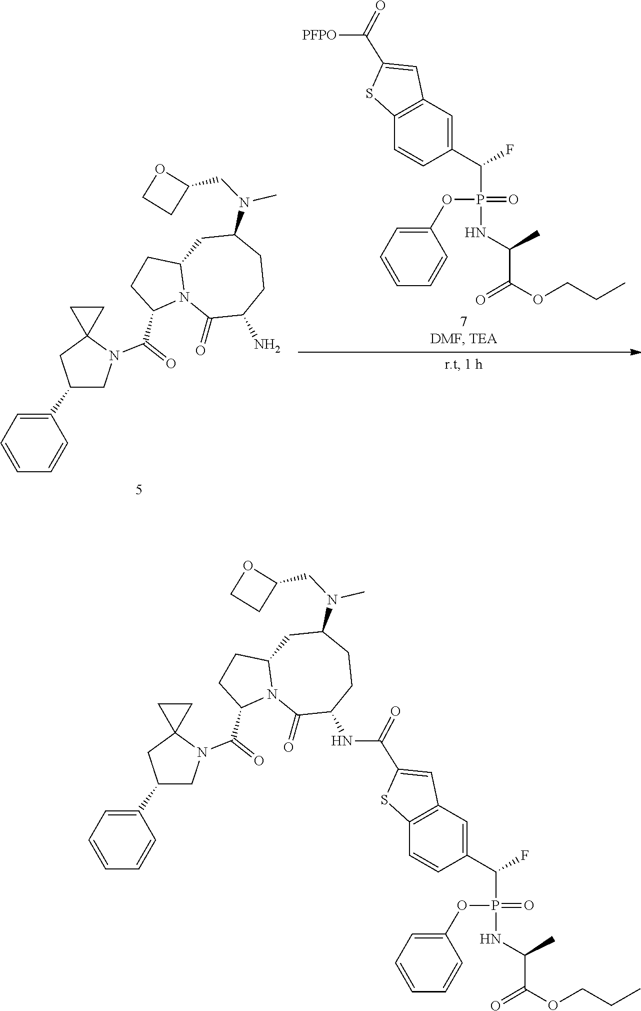

To a solution of (3S,6S,9S,10aR)-6-amino-9-(methyl(((S)-oxetan-2-yl)methyl)amino)-3-((R)-6-phenyl-4-azaspiro[2.4]heptane-4-carbonyl)octahydropyrrolo[1,2-a]azocin-5(1H)-one (5, 45 mg, TFA salt) in DMF (2 mL) were added TEA (23.51 mg. 232.37 mol, 3 eq.) and perfluorophenyl 5-((1R)-fluoro((((S)-1-oxo-1-propoxypropan-2-yl)amino)(phenoxy)phosphoryl)methyl)benzo[b]thiophene-2-carboxylate (7, 50 mg, 77.46 mol, 1 eq.), and the resulting mixture was stirred at room temperature for 1 h. The crude product was purified by Prep-HPLC to afford propyl (((R)-fluoro(2-(((3S,6S,9S,10aR)-9-(methyl(((S)-oxetan-2-yl)methyl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C18, 7.2 mg, 7.64 mol, 9.8%) as a white solid. LCMS (ESI): m/z=943.0 [M+H]⁺. ¹H NMR (400 MHz, DMSO-d₆) δ (ppm) 8.79-8.68 (m, 1H), 8.27 (s, 1H), 8.11-8.01 (m, 2H), 7.64-7.55 (m, 1H), 7.39-7.25 (m, 6H), 7.23-7.09 (m, 4H), 6.26-6.01 (m, 2H), 4.83-4.70 (m, 2H), 4.54-4.42 (m, 2H), 4.41-4.25 (m, 2H), 4.15-4.08 (m, 1H), 3.96-3.69 (m, 4H), 3.59-3.49 (m, 1H), 2.95-2.84 (m, 1H), 2.70-2.55 (m, 2H), 2.42-1.35 (m, 21H), 1.13 (d, J=7.1 Hz, 3H), 0.84-0.71 (m, 3H), 0.57-0.41 (m, 2H). ¹⁹F NMR (376 MHz, DMSO-d₆) δ (ppm) −194.25 (d, J=83.0 Hz), −196.30 (d, J=85.7 Hz). ³¹P NMR (162 MHz, DMSO-d₆) δ (ppm) 17.32 (d, J=82.8 Hz), 17.07 (d, J=85.3 Hz).

### Synthesis of ((S)-fluoro(2-(((3S,6S,9S,10aR)-9-morpholino-5-oxo-3-((R)-6-(pyridin-3-yl)-4-azaspiro[2.4]heptane-4-carbonyl)decahydropyrrolo[1,2-a]azocin-6-yl)carbamoyl)benzo[b]thiophen-5-yl)methyl)phosphonic acid (Compound A10) and propyl ((S)-((S)-fluoro(2-(((3S,6S,9S,10aR)-9-morpholino-5-oxo-3-((R)-6-(pyridin-3-yl)-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C43)

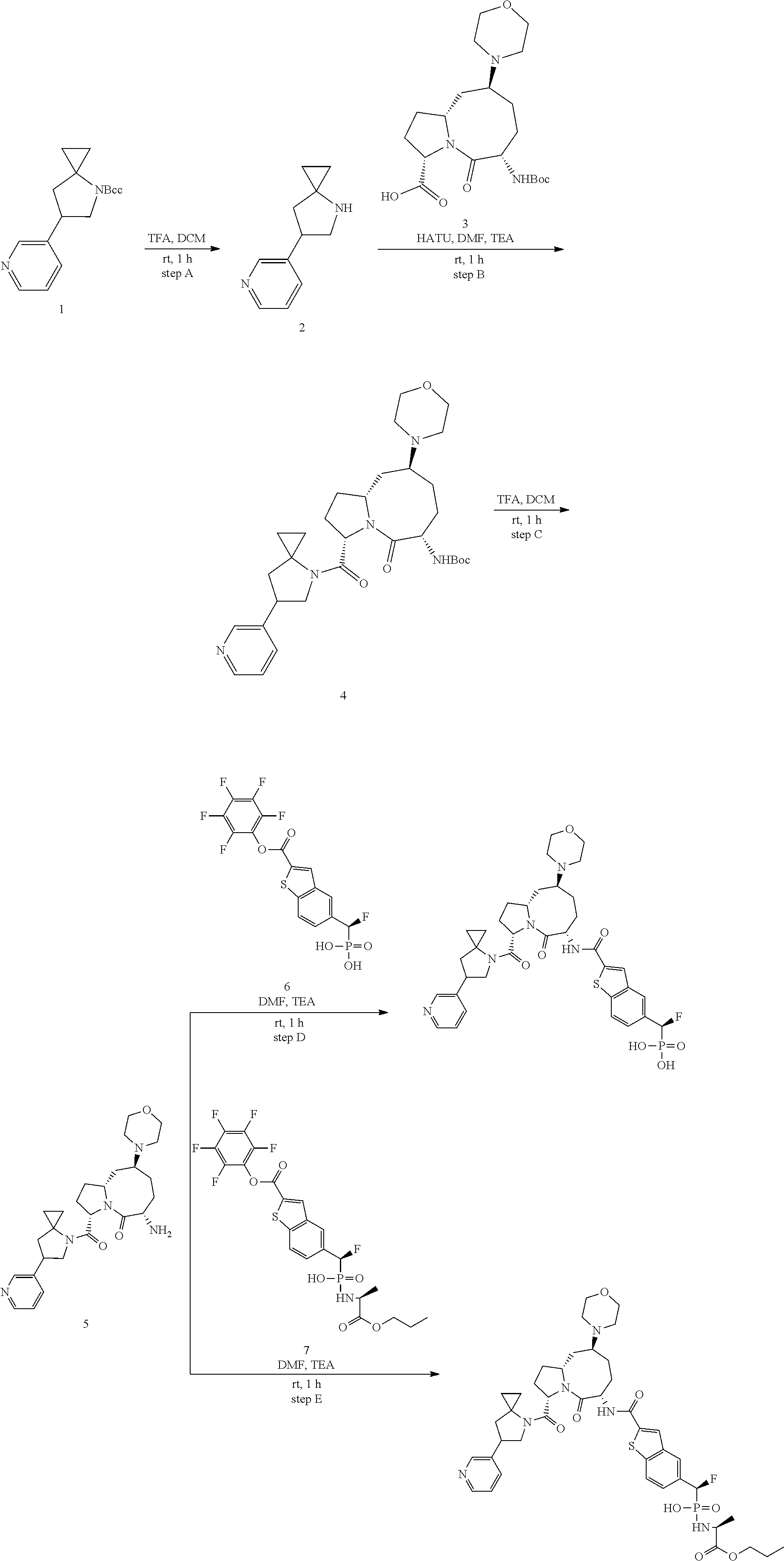

### Step A: (R)-6-(pyridin-3-yl)-4-azaspiro[2.4]heptane

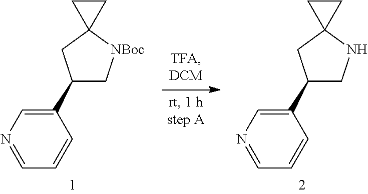 [SMILES: CC(C)(C)OC(=O)N1C[C@@H](c2cccnc2)CC12CC2 ; c1cncc([C@@H]2CNC3(CC3)C2)c1]

To a solution of tert-butyl (R)-6-(pyridin-3-yl)-4-azaspiro[2.4]heptane-4-carboxylate (1, 1 g, 3.65 mmol, 1 eq.) in DCM (10 mL) was added TFA (10 mL), The resulting mixture was stirred for 1 h at room temperature. The resulting mixture was concentrated to dryness under reduced pressure to afford crude product (R)-6-(pyridin-3-yl)-4-azaspiro[2.4]heptane (2, 1.0 g, TFA salt) as a yellow oil which was used to the next step without further purification. LCMS (ESI): m/z=175 [M+H]⁺.

### Step B: tert-butyl ((3S,6S,9S,10aR)-9-morpholino-5-oxo-3-((R)-6-(pyridin-3-yl)-4-azaspiro[2.4]heptane-4-carbonyl)decahydropyrrolo[1,2-a]azocin-6-yl)carbamate

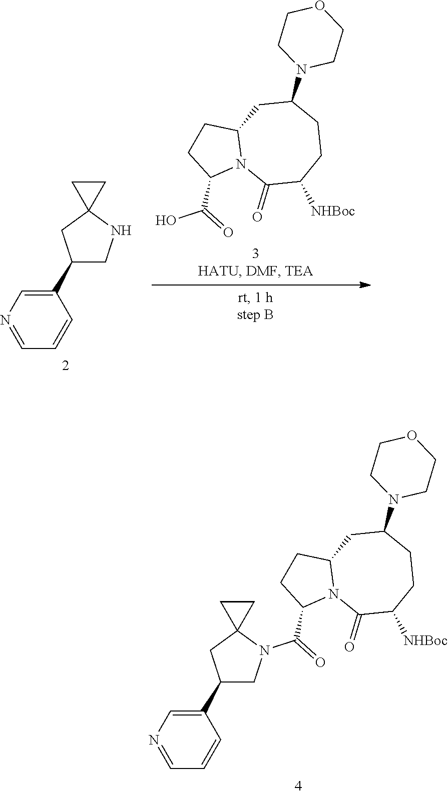 [SMILES: c1cncc([C@@H]2CNC3(CC3)C2)c1 ; CC(C)(C)OC(=O)N[C@H]1CC[C@H](N2CCOCC2)C[C@H]2CC[C@@H](C(=O)O)N2C1=O ; CC(C)(C)OC(=O)N[C@H]1CC[C@H](N2CCOCC2)C[C@H]2CC[C@@H](C(=O)N3C[C@@H](c4cccnc4)CC34CC4)N2C1=O]

To a solution of (3S,6S,9S,10aR)-6-((tert-butoxycarbonyl)amino)-9-morpholino-5-oxodecahydropyrrolo[1,2-a]azocine-3-carboxylic acid (3, 1.5 g, 3.65 mmol, 1 eq.) in DMF (10 mL) was added HATU (1.39 g, 3.65 mmol, 1 eq.), TEA (737 mg, 7.30 mmol, 2 eq). The solution was stirred for 10 min, then (R)-6-(pyridin-3-yl)-4-azaspiro[2.4]heptane (2, 1.0 g, TFA salt) was added to the solution, The resulting mixture was stirred for 1 h at room temperature. The crude product was purified by C18 column to afford tert-butyl ((3S,6S,9S,10aR)-9-morpholino-5-oxo-3-((R)-6-(pyridin-3-yl)-4-azaspiro[2.4]heptane-4-carbonyl)decahydropyrrolo[1,2-a]azocin-6-yl)carbamate (4, 1.7 g, 3.0 mmol, 82.1% yield, two steps) as a white solid. LCMS (ESI): m/z=468 [M+H]⁺.

### Step C: (3S,6S,9S,10aR)-6-amino-9-morpholino-3-((R)-6-(pyridin-3-yl)-4-azaspiro[2.4]heptane-4-carbonyl)octahydropyrrolo[1,2-a]azocin-5(1H)-one

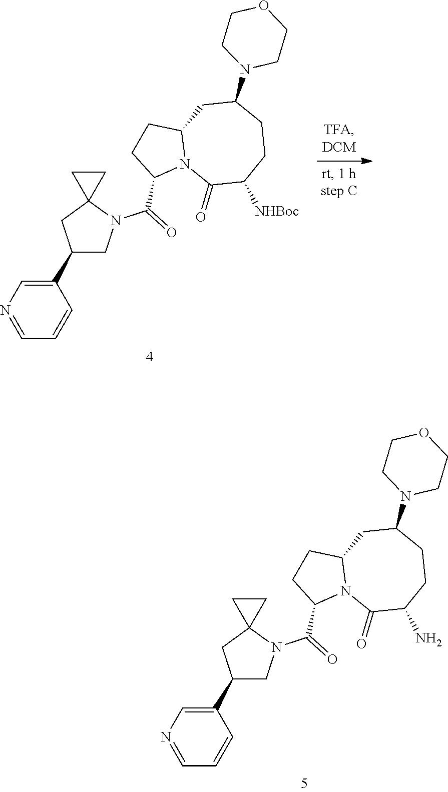 [SMILES: CC(C)(C)OC(=O)N[C@H]1CC[C@H](N2CCOCC2)C[C@H]2CC[C@@H](C(=O)N3C[C@@H](c4cccnc4)CC34CC4)N2C1=O ; N[C@H]1CC[C@H](N2CCOCC2)C[C@H]2CC[C@@H](C(=O)N3C[C@@H](c4cccnc4)CC34CC4)N2C1=O]

To a solution of tert-butyl ((3S,6S,9S,10aR)-9-morpholino-5-oxo-3-((R)-6-(pyridin-3-yl)-4-azaspiro[2.4]heptane-4-carbonyl)decahydropyrrolo[1,2-a]azocin-6-yl)carbamate (4, 20 mg, 0.035 mmol) in DCM (2 mL) was added TFA (1 mL) and the resulting mixture was stirred for 1 h at room temperature. The resulting mixture was concentrated to dryness under reduced pressure to afford crude product (3S,6S,9S,10aR)-6-amino-9-morpholino-3-((R)-6-(pyridin-3-yl)-4-azaspiro[2.4]heptane-4-carbonyl)octahydropyrrolo[1,2-a]azocin-5(1H)-one (5, 20 mg, TFA salt) as a yellow oil which was used to the next step without further purification. LCMS (ESI): m/z=568 [M+H]⁺.

### Step D: ((S)-fluoro(2-(((3S,6S,9S,10aR)-9-morpholino-5-oxo-3-((R)-6-(pyridin-3-yl)-4-azaspiro[2.4]heptane-4-carbonyl)decahydropyrrolo[1,2-a]azocin-6-yl)carbamoyl)benzo[b]thiophen-5-yl)methyl)phosphonic acid

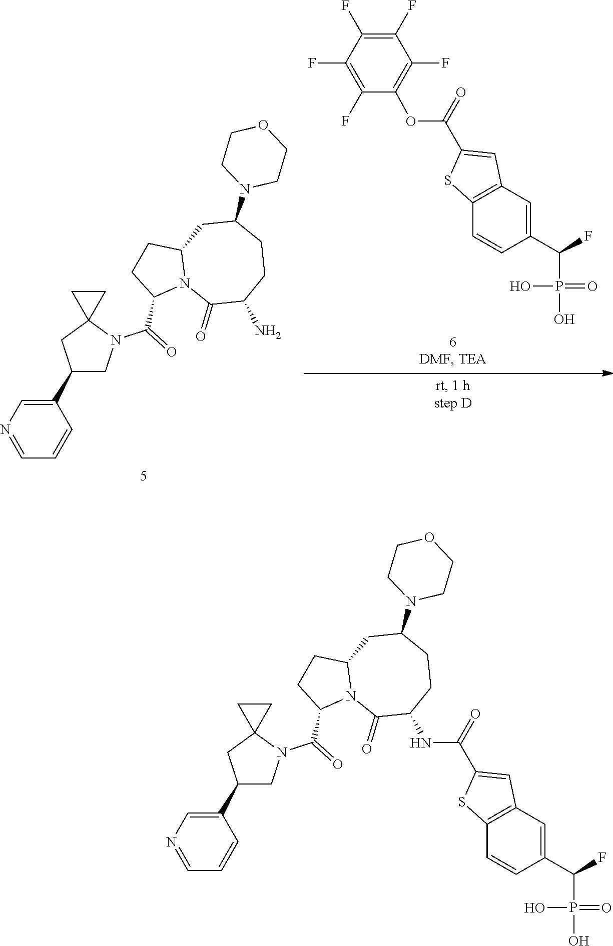 [SMILES: N[C@H]1CC[C@H](N2CCOCC2)C[C@H]2CC[C@@H](C(=O)N3C[C@@H](c4cccnc4)CC34CC4)N2C1=O ; O=C(Oc1c(F)c(F)c(F)c(F)c1F)c1cc2cc([C@@H](F)P(=O)(O)O)ccc2s1 ; O=C(N[C@H]1CC[C@H](N2CCOCC2)C[C@H]2CC[C@@H](C(=O)N3C[C@@H](c4cccnc4)CC34CC4)N2C1=O)c1cc2cc([C@@H](F)P(=O)(O)O)ccc2s1]

To a solution of (3S,6S,9S,10aR)-6-amino-9-morpholino-3-((R)-6-(pyridin-3-yl)-4-azaspiro[2.4]heptane-4-carbonyl)octahydropyrrolo[1,2-a]azocin-5(1H)-one (5, 20 mg, TFA salt) was added TEA (7.1 mg, 0.07 mmol, 2 eq.), (S)-(fluoro(2-((perfluorophenoxy)carbonyl)benzo[b]thiophen-5-yl)methyl)phosphonic acid (6, 16.1 mg, 0.035 mml, 1 eq.). The resulting mixture was stirred for 1 h at room temperature. The crude product was purified by Prep-HPLC to afford ((S)-fluoro(2-(((3S,6S,9S,10aR)-9-morpholino-5-oxo-3-((R)-6-(pyridin-3-yl)-4-azaspiro[2.4]heptane-4-carbonyl)decahydropyrrolo[1,2-a]azocin-6-yl)carbamoyl)benzo[b]thiophen-5-yl)methyl)phosphonic acid (Compound A10, 10 mg, 0.014 mmol) as a white solid. LCMS (ESI): m/z=740 [M+H]⁺. ¹H NMR (400 MHz, DMSO-d₆) δ (ppm) 8.82-8.74 (m, 1H), 8.61 (s, 1H), 8.51-8.43 (m, 1H), 8.24 (s, 1H), 8.06-7.95 (m, 2H), 7.89-7.81 (m, 1H), 7.57-7.49 (m, 1H), 7.43-7.35 (m, 1H), 5.91-5.74 (m, 1H), 4.85-4.78 (m, 1H), 4.56 (t, J=8.5 Hz, 2H), 4.19-4.11 (m, 2H), 4.06-4.01 (m, 2H), 3.84-3.78 (m, 2H), 3.73-3.69 (m, 1H), 3.62-3.55 (m, 2H), 3.28-3.22 (m, 2H), 3.15-3.08 (m, 1H), 2.25-1.95 (m, 9H), 1.87-1.61 (m, 5H), 0.56-0.44 (m, 2H). ¹⁹F NMR (377 MHz, DMSO-d₆) δ (ppm) −194.71 (d, J=81.3 Hz). ³¹P NMR (162 MHz, DMSO-d₆) δ (ppm) 10.92 (d, J=81.1 Hz).

### Step E: propyl ((S)-((S)-fluoro(2-(((3S,6S,9S,10aR)-9-morpholino-5-oxo-3-((R)-6-(pyridin-3-yl)-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

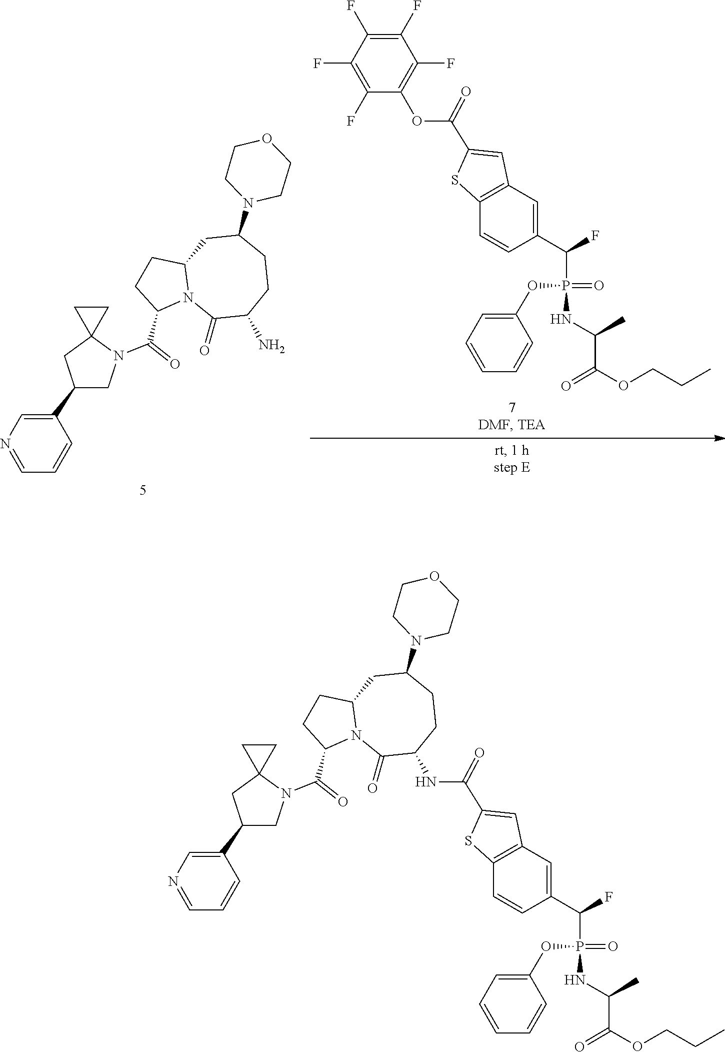 [SMILES: N[C@H]1CC[C@H](N2CCOCC2)C[C@H]2CC[C@@H](C(=O)N3C[C@@H](c4cccnc4)CC34CC4)N2C1=O ; CCCOC(=O)[C@H](C)N[P@@](=O)(Oc1ccccc1)[C@H](F)c1ccc2sc(C(=O)Oc3c(F)c(F)c(F)c(F)c3F)cc2c1 ; CCCOC(=O)[C@H](C)N[P@@](=O)(Oc1ccccc1)[C@H](F)c1ccc2sc(C(=O)N[C@H]3CC[C@H](N4CCOCC4)C[C@H]4CC[C@@H](C(=O)N5C[C@@H](c6cccnc6)CC56CC6)N4C3=O)cc2c1]

To a solution of (3S,6S,9S,10aR)-6-amino-9-morpholino-3-((R)-6-(pyridin-3-yl)-4-azaspiro[2.4]heptane-4-carbonyl)octahydropyrrolo[1,2-a]azocin-5(1H)-one (5, 20 mg, TFA salt) was added TEA (7.1 mg, 0.07 mmol, 2 eq.), perfluorophenyl 5-((S)-fluoro((S)-(((S)-1-oxo-1-propoxypropan-2-yl)amino)(phenoxy)phosphoryl)methyl)benzo[b]thiophene-2-carboxylate (7, 22.5 mg, 0.035 mml, 1 eq.). The resulting mixture was stirred for 1 h at room temperature. The crude product was purified by Prep-HPLC to afford propyl ((S)-((S)-fluoro(2-(((3S,6S,9S,10aR)-9-morpholino-5-oxo-3-((R)-6-(pyridin-3-yl)-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C43, 15 mg, 0.016 mmol, 57% yield) as a white solid. LCMS (ESI): m/z=929 [M+H]⁺. ¹H NMR (400 MHz, DMSO-d₆) δ (ppm) 8.79-8.65 (m, 1H), 8.63-8.51 (m, 1H), 8.46-8.38 (m, 1H), 8.28 (s, 1H), 8.14-7.99 (m, 2H), 7.82-7.73 (m, 1H), 7.65-7.56 (m, 1H), 7.41-7.29 (m, 3H), 7.24-7.14 (m, 3H), 6.25-6.03 (m, 2H), 5.04-4.87 (m, 1H), 4.56-4.44 (m, 1H), 4.37-4.24 (m, 1H), 4.20-4.06 (m, 1H), 3.98-3.85 (m, 2H), 3.84-3.49 (m, 7H), 2.80-2.69 (m, 1H), 2.48-1.57 (m, 18H), 1.54-1.42 (m, 2H), 0.98-0.89 (m, 3H), 0.85-0.74 (m, 3H), 0.56-0.37 (m, 2H). ¹⁹F NMR (376 MHz, DMSO-d₆) δ (ppm) −194.13 (d, J=82.5 Hz). ³¹P NMR (162 MHz, DMSO-d₆) δ (ppm) 16.93 (d, J=82.5 Hz).

### Synthesis of propyl (((R)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-D-alaninate (Compound C84)

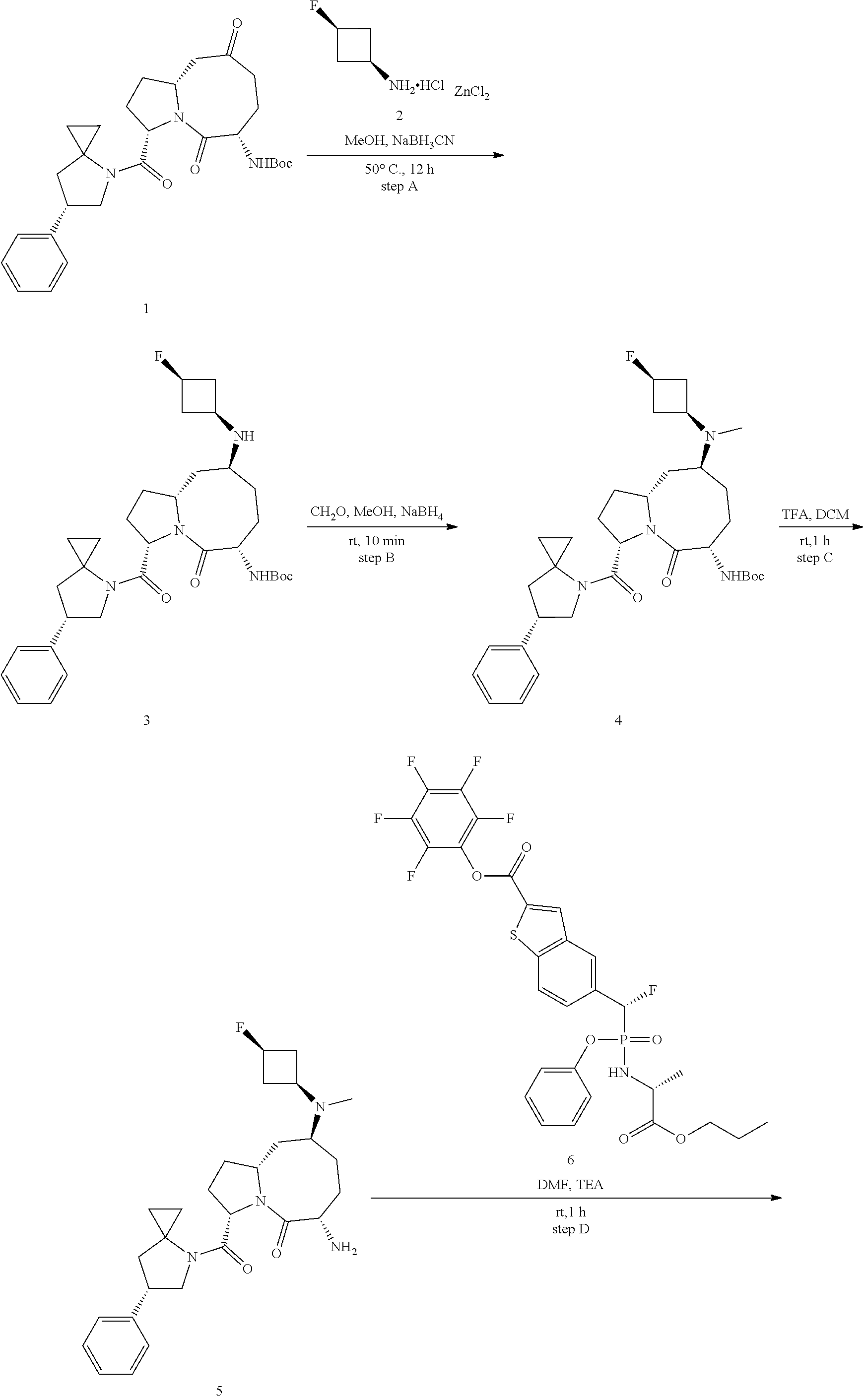

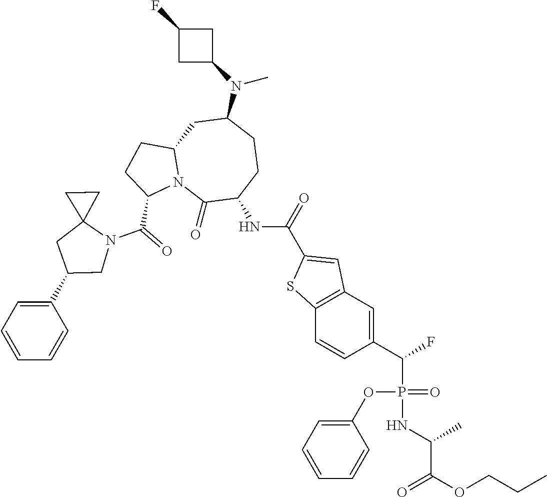 [SMILES: CCCOC(=O)[C@@H](C)NP(=O)(Oc1ccccc1)[C@@H](F)c1ccc2sc(C(=O)N[C@H]3CC[C@H](N(C)[C@H]4C[C@@H](F)C4)C[C@H]4CC[C@@H](C(=O)N5C[C@H](c6ccccc6)CC56CC6)N4C3=O)cc2c1]

### Step A: tert-butyl ((3S,6S,9S,10aR)-9-(((1s,3R)-3-fluorocyclobutyl)amino)-5-oxo-3-((S)-6-phenyl-4-azaspiro[2.4]heptane-4-carbonyl)decahydropyrrolo[1,2-a]azocin-6-yl)carbamate

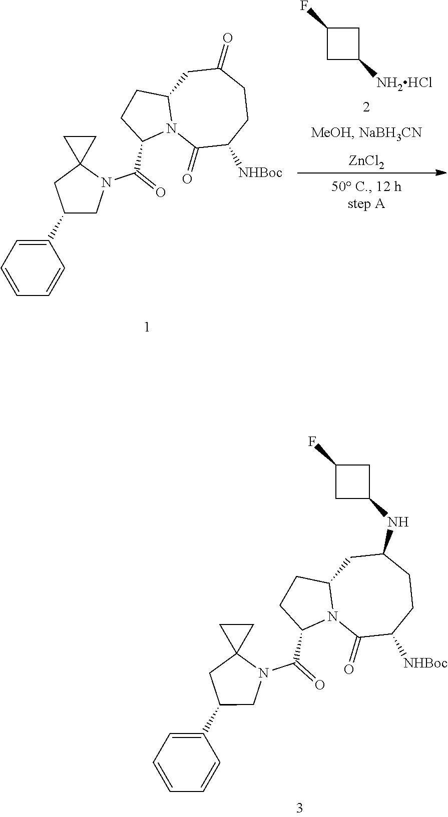

To a solution of tert-butyl ((3S,6S,10aR)-5,9-dioxo-3-((S)-6-phenyl-4-azaspiro[2.4]heptane-4-carbonyl)decahydropyrrolo[1,2-a]azocin-6-yl)carbamate (1, 3 g, 6.06 mmol, 1 eq.) in MeOH (60 mL) was added (1s,3s)-3-fluorocyclobutan-1-amine hydrochloride (2, 1.14 g, 9.09 mmol, 1.5 eq.), ZnCl₂ (8.3 g, 60.6 mmol, 10 eq.), the solution was stirred for 2 hr at 50° C. under the atmosphere of N₂. Then NaBH₃CN (3.81 g, 60.6 mmol, 10 eq.) was added portion wise every 30 mins (2 eq. (762 mg each time) added 5 times). The resulting mixture was stirred for 12 hr at 50° C., then cooled to room temperature, The solution was used to next step without further workup. LCMS (ESI): m/z=569 [M+H]⁺.

### Step B: tert-butyl ((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamate

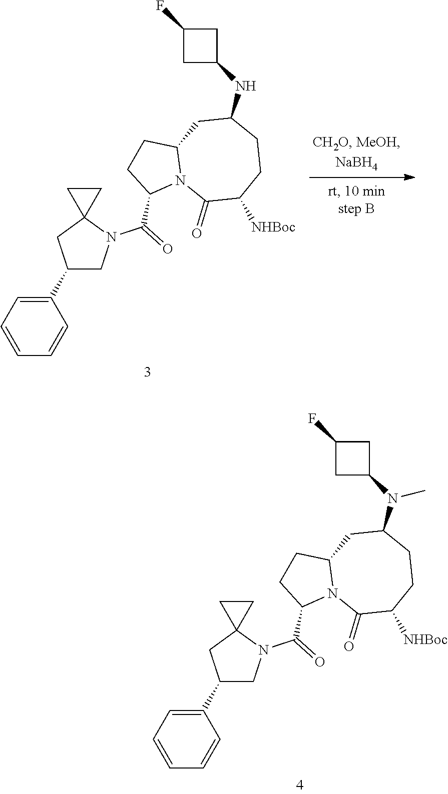 [SMILES: CC(C)(C)OC(=O)N[C@H]1CC[C@H](N[C@H]2C[C@@H](F)C2)C[C@H]2CC[C@@H](C(=O)N3C[C@H](c4ccccc4)CC34CC4)N2C1=O ; CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](F)C1]

To the above mixture (step A) was added 40% formaldehyde aqueous (3 mL) at room temperature, the mixture was stirred for 30 min, then NaBH₄ (1.15 g, 30.3 mmol, 5 eq.) was added in portions, the mixture was stirred for another 30 mins. The reaction was quenched by NH₄Cl (sat. aq), diluted with H₂O (200 mL) and extracted with EtOAc (100 mL×3), the organic solution was dried over Na₂SO₄, then concentrated under vacuum, the crude product was purified by C18 column (MeCN:H₂O (0.1% ammonium hydroxide)=5%-70%) to afford tert-butyl ((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamate (4, 3 g, 5.15 mmol, 85% yield two steps) as a white solid. LCMS (ESI): m/z=583 [M+H]⁺.

### Step C: (3S,6S,9S,10aR)-6-amino-9-(((1s,3R)-3-fluorocyclobutyl)(methyl)amino)-3-((S)-6-phenyl-4-azaspiro[2.4]heptane-4-carbonyl)octahydropyrrolo[1,2-a]azocin-5(1H)-one

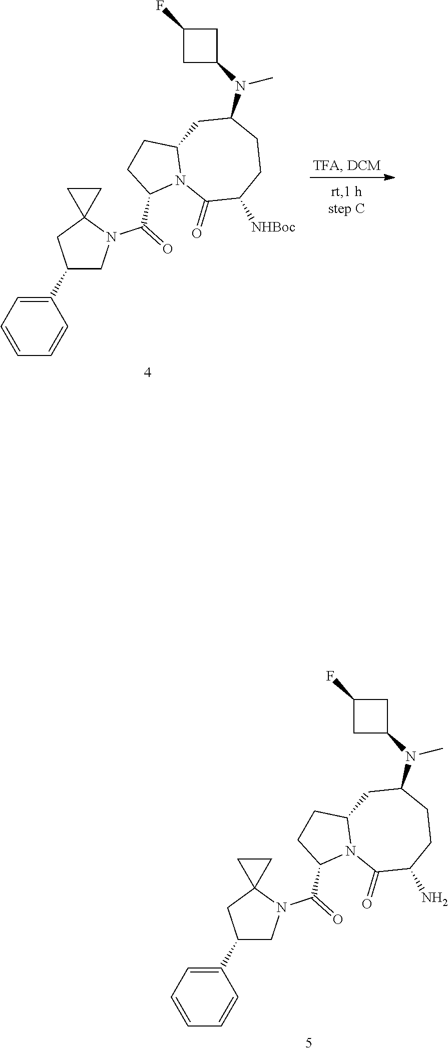 [SMILES: CN([C@H]1CC[C@H](NC(=O)OC(C)(C)C)C(=O)N2[C@H](CC[C@H]2C(=O)N2C[C@H](c3ccccc3)CC23CC3)C1)[C@H]1C[C@@H](F)C1 ; CN([C@H]1CC[C@H](N)C(=O)N2[C@H](CC[C@H]2C(=O)N2C[C@H](c3ccccc3)CC23CC3)C1)[C@H]1C[C@@H](F)C1]

To a solution of tert-butyl((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamate (4, 20 mg, 0.034 mmol) in DCM (2 mL) was added TFA (2 mL). The resulting mixture was stirred for 1 h at room temperature. The resulting mixture was concentrated to dryness under reduced pressure to afford crude product (3S,6S,9S,10aR)-6-amino-9-(((1s,3R)-3-fluorocyclobutyl)(methyl)amino)-3-((S)-6-phenyl-4-azaspiro[2.4]heptane-4-carbonyl)octahydropyrrolo[1,2-a]azocin-5(1H)-one (5, 20 mg, TFA salt) as a yellow oil which was used to the next step without further purification. LCMS (ESI): m/z=483 [M+H]⁺.

### Step D: propyl (((R)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-D-alaninate

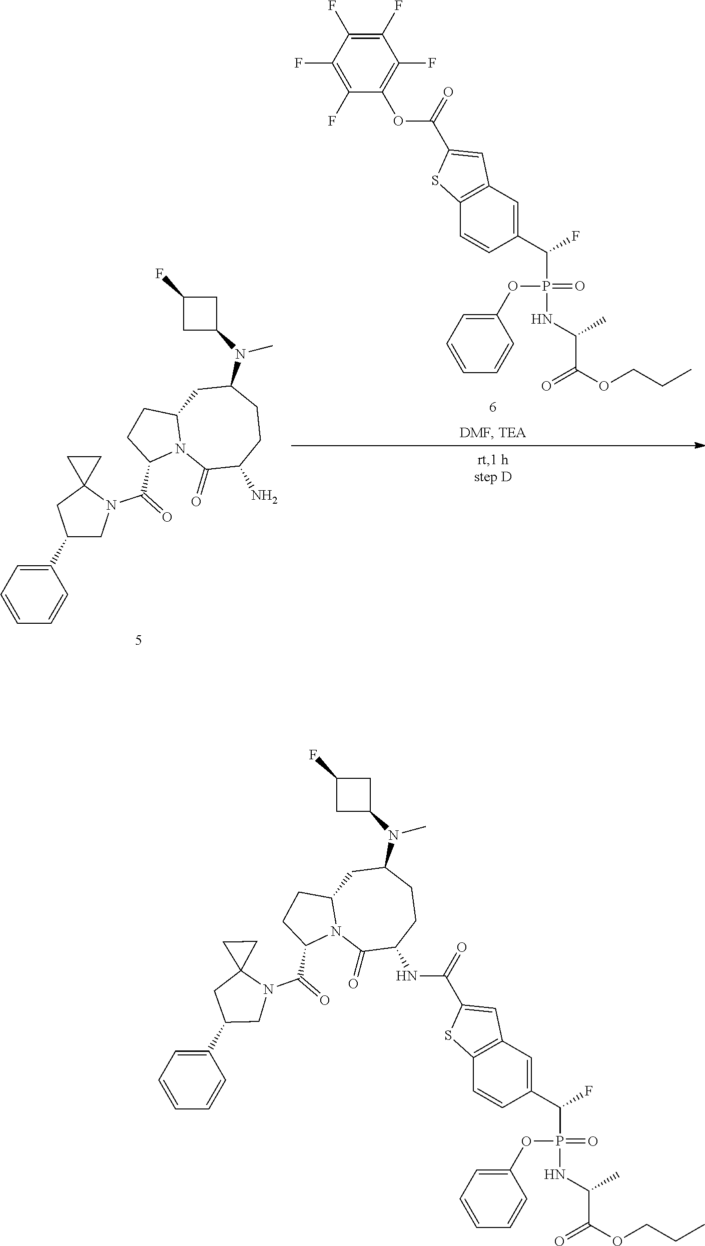 [SMILES: CN([C@H]1CC[C@H](N)C(=O)N2[C@H](CC[C@H]2C(=O)N2C[C@H](c3ccccc3)CC23CC3)C1)[C@H]1C[C@@H](F)C1 ; CCCOC(=O)[C@@H](C)NP(=O)(Oc1ccccc1)[C@@H](F)c1ccc2sc(C(=O)Oc3c(F)c(F)c(F)c(F)c3F)cc2c1 ; CCCOC(=O)[C@@H](C)NP(=O)(Oc1ccccc1)[C@@H](F)c1ccc2sc(C(=O)N[C@H]3CC[C@H](N(C)[C@H]4C[C@@H](F)C4)C[C@H]4CC[C@@H](C(=O)N5C[C@H](c6ccccc6)CC56CC6)N4C3=O)cc2c1]

To a solution of (3S,6S,9S,10aR)-6-amino-9-(((1s,3R)-3-fluorocyclobutyl)(methyl)amino)-3-((S)-6-phenyl-4-azaspiro[2.4]heptane-4-carbonyl)octahydropyrrolo[1,2-a]azocin-5(1H)-one (5, 20 mg, TFA salt) in DMF (2 mL) was added TEA (6.9 mg, 0.068 mmol, 2 eq.), perfluorophenyl 5-((1R)-fluoro((((R)-1-oxo-1-propoxypropan-2-yl)amino)(phenoxy)phosphoryl)methyl)benzo[b]thiophene-2-carboxylate (7, 22.2 mg, 0.034 mmol, 1 eq.). The resulting mixture was stirred for 1 h at room temperature, LCMS shows that the reaction was completed. The crude product was purified by Prep-HPLC to afford propyl (((R)-fluoro(2-(((3S,6S,9S,10aR)-9-(((1s,3R)-3-fluorocyclobutyl)(methyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-D-alaninate (Compound C84, 21 mg) as a white solid. LCMS (ESI): m/z=944 [M+H]⁺. 1H NMR (400 MHz, DMSO-d₆) δ (ppm) 8.85-8.73 (m, 1H), 8.27 (d, J=4.2 Hz, 1H), 8.11-8.01 (m, 2H), 7.64-7.54 (m, 1H), 7.40-7.27 (m, 6H), 7.24-7.11 (m, 4H), 6.30-6.08 (m, 2H), 4.91-4.65 (m, 2H), 4.52-4.41 (m, 1H), 4.29 (s, 1H), 4.17-4.07 (m, 1H), 3.96-3.67 (m, 4H), 3.59-3.46 (m, 1H), 3.05-2.93 (m, 1H), 2.64-2.54 (m, 1H), 2.44-2.30 (m, 2H), 2.27-2.03 (m, 3H), 2.02-1.92 (m, 6H), 1.89-1.75 (m, 3H), 1.74-1.43 (m, 8H), 1.19-0.94 (m, 3H), 0.84-0.74 (m, 3H), 0.55-0.40 (m, 2H). ¹⁹F NMR (376 MHz, DMSO-d₆) δ (ppm) −165.28 (s), −194.23 (d, J=82.5 Hz), −196.21 (d, J=86.3 Hz). ³¹P NMR (162 MHz, DMSO-d₆) δ (ppm) 17.36 (d, J=86.2 Hz), 16.87 (d, J=82.3 Hz).

### Synthesis of propyl (((R)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C100) and ((R)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A43)

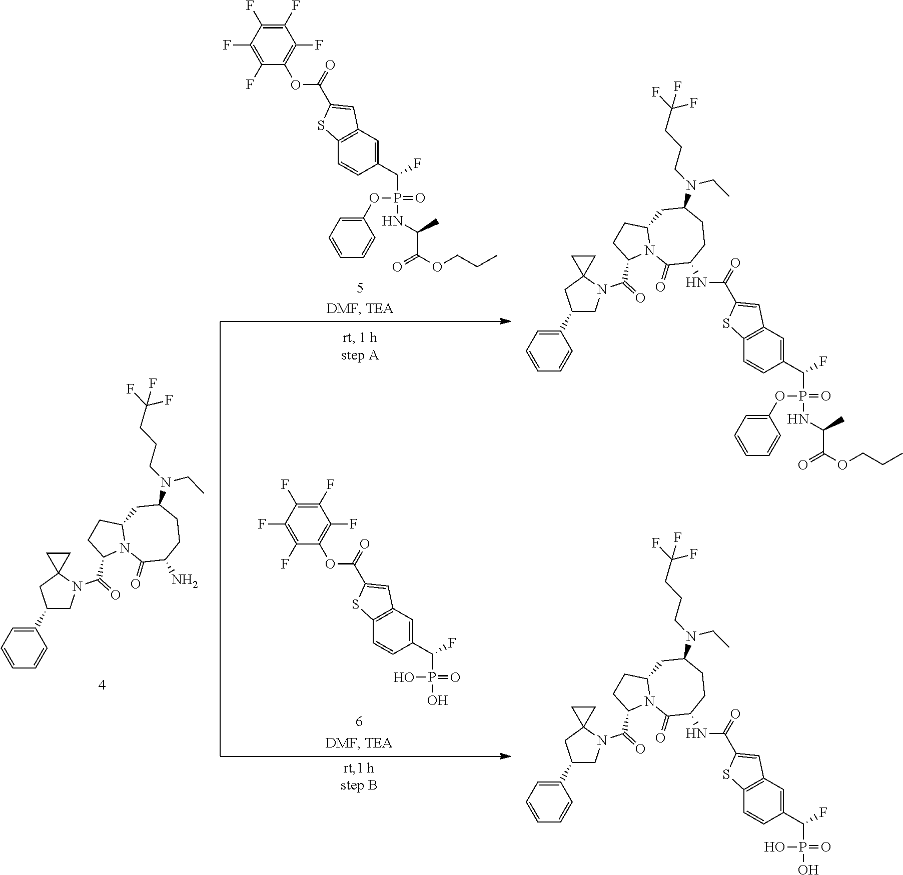

### Step A: propyl (((R)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate

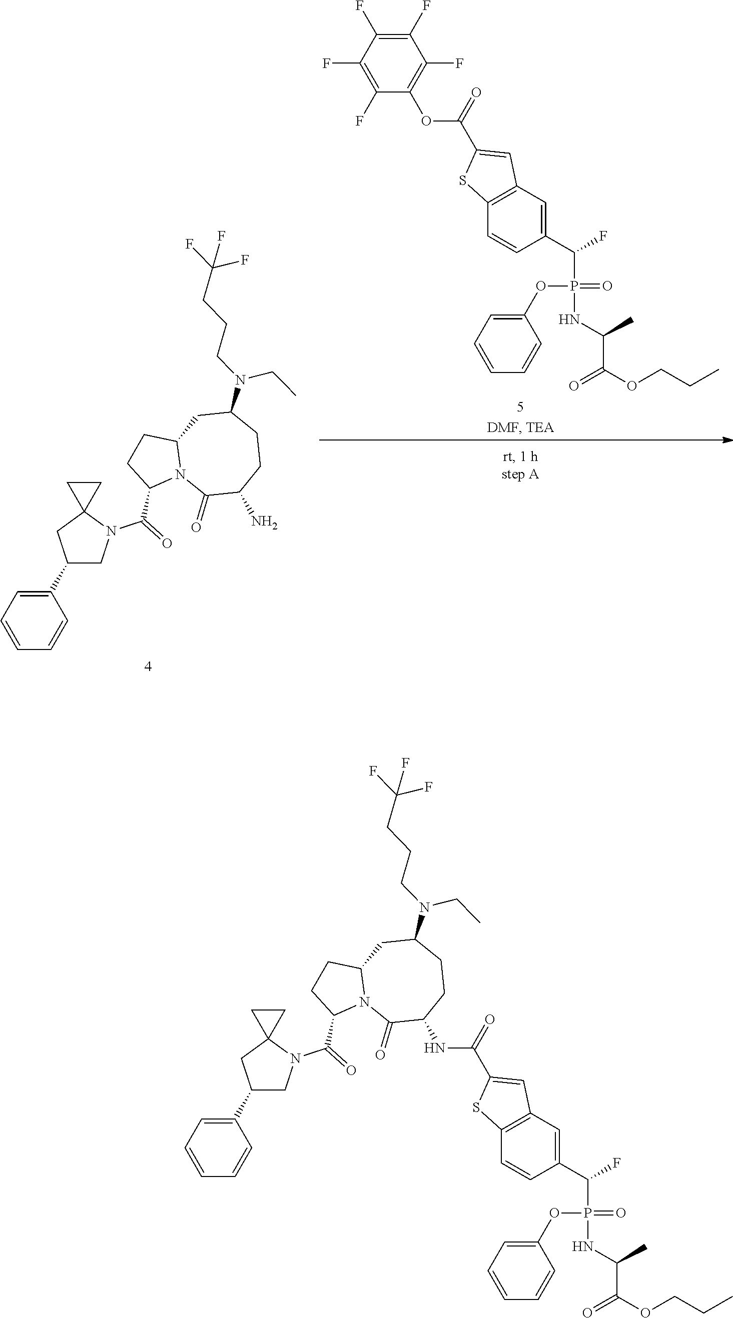 [SMILES: CCN(CCCC(F)(F)F)[C@H]1CC[C@H](N)C(=O)N2[C@H](CC[C@H]2C(=O)N2C[C@H](c3ccccc3)CC23CC3)C1 ; CCCOC(=O)[C@H](C)NP(=O)(Oc1ccccc1)[C@@H](F)c1ccc2sc(C(=O)Oc3c(F)c(F)c(F)c(F)c3F)cc2c1 ; CCCOC(=O)[C@H](C)NP(=O)(Oc1ccccc1)[C@@H](F)c1ccc2sc(C(=O)N[C@H]3CC[C@H](N(CC)CCCC(F)(F)F)C[C@H]4CC[C@@H](C(=O)N5C[C@H](c6ccccc6)CC56CC6)N4C3=O)cc2c1]

To a solution of (3S,6S,9S,10aR)-6-amino-9-(ethyl(4,4,4-trifluorobutyl)amino)-3-((S)-6-phenyl-4-azaspiro[2.4]heptane-4-carbonyl)octahydropyrrolo[1,2-a]azocin-5(1H)-one (4, 30 mg, TFA salt) in DMF (2 mL) was added TEA (14.3 mg, 0.141 mmol, 3 eq.), perfluorophenyl 5-((1R)-fluoro((((S)-1-oxo-1-propoxypropan-2-yl)amino)(phenoxy)phosphoryl)methyl)benzo[b]thiophene-2-carboxylate (5, 30 mg, 0.046 mmol, 1 eq). The resulting mixture was stirred for 1 h at room temperature. The crude product was purified by Prep-HPLC to afford propyl (((R)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C100, 35 mg, 0.035 mmol, 76.1%) as a white solid. LCMS (ESI): m/z=996.6 [M+H]⁺. ¹H NMR (400 MHz, DMSO-d₆) δ (ppm) 8.82-8.75 (m, 1H), 8.27 (s, 1H), 8.09-8.03 (m, 2H), 7.63-7.57 (m, 1H), 7.38-7.27 (m, 6H), 7.24-7.14 (m, 4H), 6.30-6.04 (m, 2H), 4.78-4.67 (m, 1H), 4.50-4.42 (m, 1H), 4.33-4.25 (m, 1H), 4.16-4.06 (m, 1H), 3.95-3.67 (m, 4H), 3.58-3.48 (m, 1H), 3.07-2.97 (m, 1H), 2.48-2.09 (m, 9H), 2.04-1.92 (m, 3H), 1.84-1.39 (m, 12H), 1.16-1.08 (m, 3H), 0.96 (t, J=7.0 Hz, 3H), 0.86-0.74 (m, 3H), 0.53-0.38 (m, 2H). ¹⁹F NMR (377 MHz, DMSO-d₆) δ (ppm) −64.51 (s), −194.30 (d, J=83.0 Hz), −196.31 (d, J=85.8 Hz). ³¹P NMR (162 MHz, DMSO-d₆) δ (ppm) 17.32 (d, J=82.6 Hz), 17.07 (d, J=86.1 Hz). (freebase).

### Step B: ((R)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid

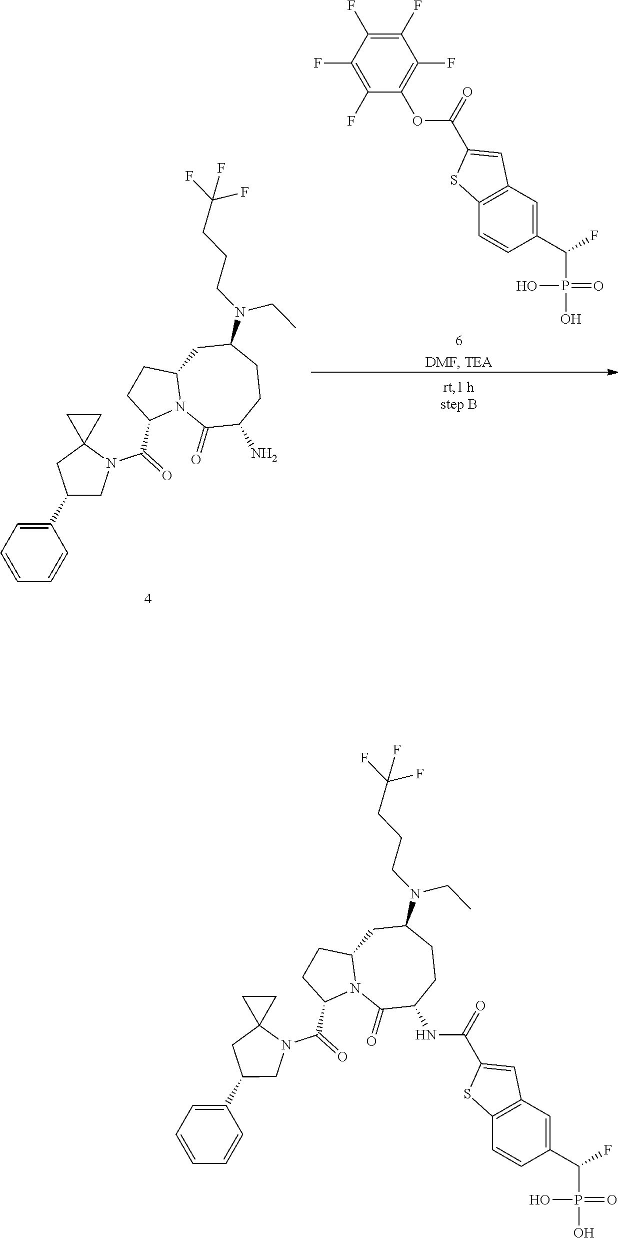 [SMILES: CCN(CCCC(F)(F)F)[C@H]1CC[C@H](N)C(=O)N2[C@H](CC[C@H]2C(=O)N2C[C@H](c3ccccc3)CC23CC3)C1 ; O=C(Oc1c(F)c(F)c(F)c(F)c1F)c1cc2cc([C@H](F)P(=O)(O)O)ccc2s1 ; CCN(CCCC(F)(F)F)[C@H]1CC[C@H](NC(=O)c2cc3cc([C@H](F)P(=O)(O)O)ccc3s2)C(=O)N2[C@H](CC[C@H]2C(=O)N2C[C@H](c3ccccc3)CC23CC3)C1]

Procedure for 4 is described in the synthesis of Compound C101 and Compound A44. To a solution of (3S,6S,9S,10aR)-6-amino-9-(ethyl(4,4,4-trifluorobutyl)amino)-3-((S)-6-phenyl-4-azaspiro[2.4]heptane-4-carbonyl)octahydropyrrolo[1,2-a]azocin-5(1H)-one (4, 30 mg, TFA salt) in DMF (2 mL) was added TEA (14.3 mg, 0.141 mmol, 3 eq.), (R)-(fluoro(2-((perfluorophenoxy)carbonyl)benzo[b]thiophen-5-yl)methyl)phosphonic acid (6, 21 mg, 0.046 mmol, 1 eq). The resulting mixture was stirred for 1 h at room temperature. The crude product was purified by Prep-HPLC to afford propyl ((R)-(2-(((3S,6S,9S,10aR)-9-(ethyl(4,4,4-trifluorobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A43, 23 mg, 0.028 mmol, 60.9%) as a white solid. LCMS (ESI): m/z=807.7 [M+H]⁺. ¹H NMR (400 MHz, DMSO-d₆) δ (ppm) 9.03-8.86 (m, 1H), 8.86-8.72 (m, 1H), 8.30-8.16 (m, 1H), 8.07-7.89 (m, 2H), 7.58-7.47 (m, 1H), 7.39-7.27 (m, 4H), 7.26-7.20 (m, 1H), 5.93-5.57 (m, 1H), 4.94-4.73 (m, 1H), 4.62-4.42 (m, 2H), 4.19-4.04 (m, 1H), 3.90-3.70 (m, 2H), 3.59-3.47 (m, 2H), 3.45-3.17 (m, 3H), 2.48-2.15 (m, 4H), 2.14-1.71 (m, 13H), 1.59-1.44 (m, 1H), 1.36-1.20 (m, 3H), 0.61-0.38 (m, 2H). ¹⁹F NMR (377 MHz, DMSO-d₆) δ (ppm) −64.75 (s), −194.80 (d, J=80.6 Hz). ³¹P NMR (162 MHz, DMSO-d₆) δ (ppm) 10.86 (d, J=80.8 Hz). (TFA salt).

### Synthesis of propyl (((R)-(2-(((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate (Compound C99) and ((R)-(2-(((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A42)

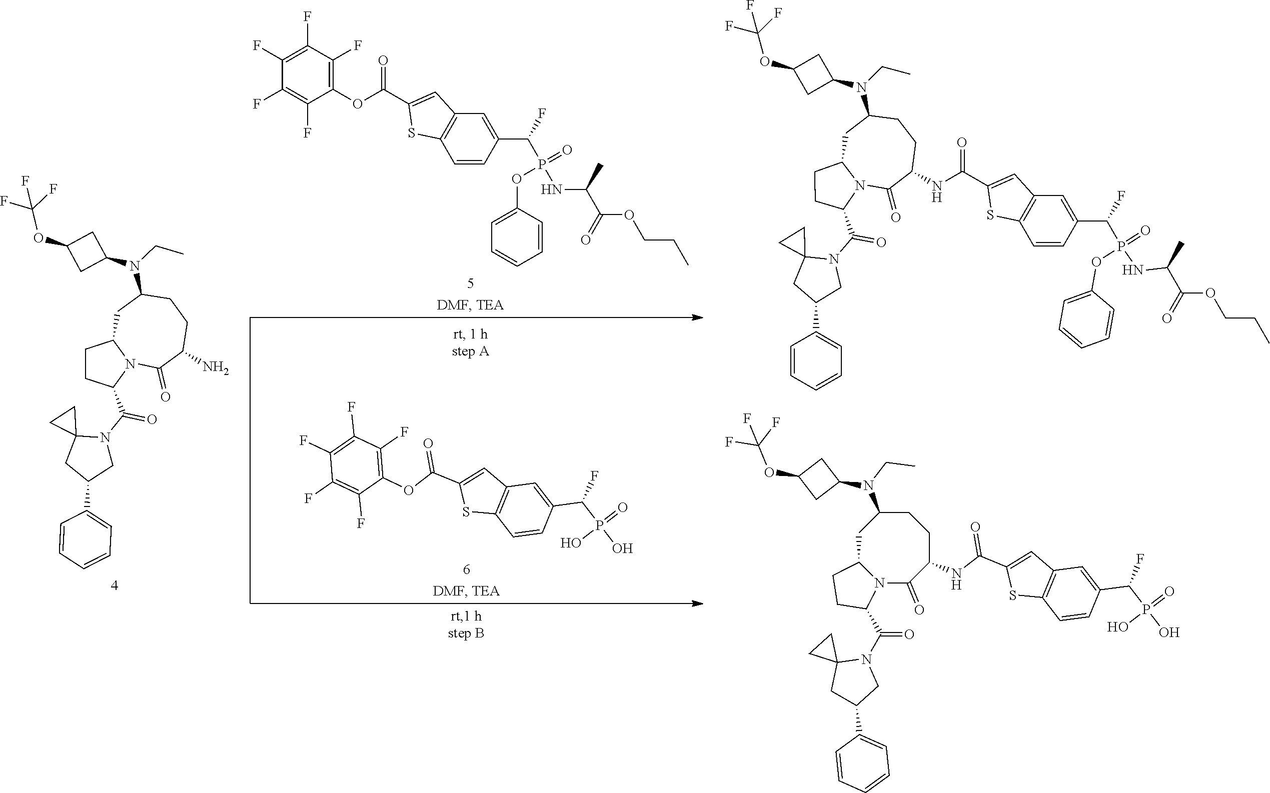

### STEP A: propyl (((R)-(2-(((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)(phenoxy)phosphoryl)-L-alaninate

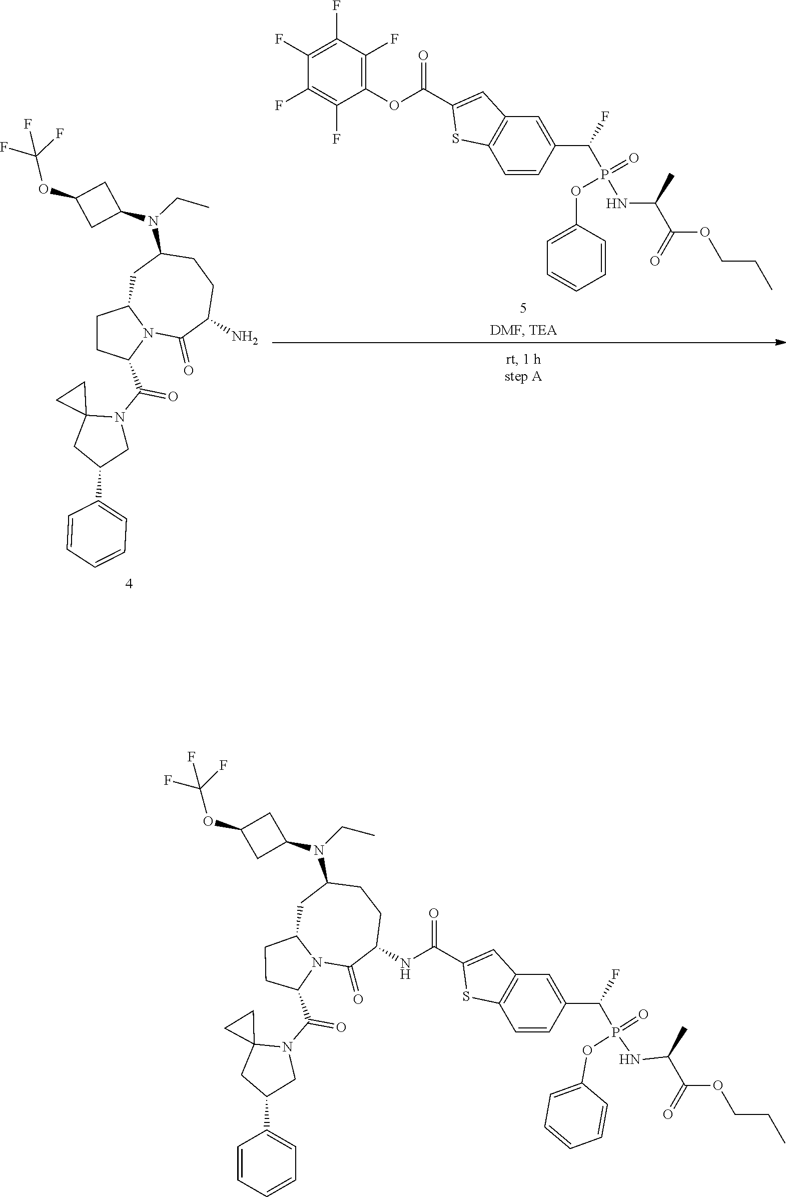 [SMILES: CCN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1 ; CCCOC(=O)[C@H](C)NP(=O)(Oc1ccccc1)[C@@H](F)c1ccc2sc(C(=O)Oc3c(F)c(F)c(F)c(F)c3F)cc2c1 ; CCCOC(=O)[C@H](C)NP(=O)(Oc1ccccc1)[C@@H](F)c1ccc2sc(C(=O)N[C@H]3CC[C@H](N(CC)[C@H]4C[C@@H](OC(F)(F)F)C4)C[C@H]4CC[C@@H](C(=O)N5C[C@H](c6ccccc6)CC56CC6)N4C3=O)cc2c1]

Procedure for 4 is described in the syntheses of Compound C119 and Compound A47. To a solution of (3S,6S,9S,10aR)-6-amino-9-(ethyl((1s,3R)-3-(trifluoromethoxy)cyclobutyl)amino)-3-((S)-6-phenyl-4-azaspiro[12.4]heptane-4-carbonyl)octahydropyrrolo[1,2-a]azocin-5(1H)-one (4, 30 mg, TFA salt) in DMF (2 mL) was added TEA (13.7 mg, 0.135 mmol, 3 eq.), perfluorophenyl 5-((1R)-fluoro((((S)-1-oxo-1-propoxypropan-2-yl)amino)(phenoxy)phosphoryl)methyl)benzo[b]thiophene-2-carboxylate (5, 29 mg, 0.045 mmol, 1 eq). The resulting mixture was stirred for 1 h at room temperature, LCMS show that the reaction was completed. The crude product was purified by Prep-HPLC to afford propyl (((R)-(2-(((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[1b]thiophen-5-yl)fluoromethyl)(phenoxy)phosphoryl)-L-alaninate (Compound C99, 23 mg, 0.022 mmol, 48.9%) as a white solid. LCMS (ESI): m/z=1024.7 [M+H]⁺. ¹H NMR (400 MHz, DMSO-d₆) δ (ppm) 8.85-8.76 (1, 1H), 8.27 (s, 1H), 8.06 (d, J=4.8 Hz, 2H), 7.60 (d, J=7.2 Hz, 1H), 7.39-7.27 (m, 6H), 7.23-7.13 (1, 4H), 6.28-6.06 (m, 2H), 4.76-4.65 (m, 1H), 4.54-4.43 (m, 2H), 4.34-4.23 (m, 1H), 4.13 (t, J=8.7 Hz, 1H), 3.97-3.89 (m, 1H), 3.85-3.70 (m, 3H), 3.59-3.49 (m, 1H), 3.08-2.99 (m, 1H), 2.89-2.79 (m, 1H), 2.63-2.53 (m, 1H), 2.37-1.90 (m, 10H), 1.88-1.38 (m, 11H), 1.13 (d, J=7.0 Hz, 3H), 0.97 (t, J=7.1 Hz, 3H), 0.85-0.74 (m, 3H), 0.52-0.39 (m, 2H). ¹⁹F NMR (377 MHz, DMSO-d) δ (ppm) −57.60 (s), −194.29 (d, J=83.1 Hz), −196.31 (d, J=86.0 Hz). ³¹P NMR (162 MHz, DMSO-d₆) δ (ppm) 17.32 (d, J=82.5 Hz), 17.07 (d, J=86.0 Hz).

### Step B: ((R)-(2-(((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid

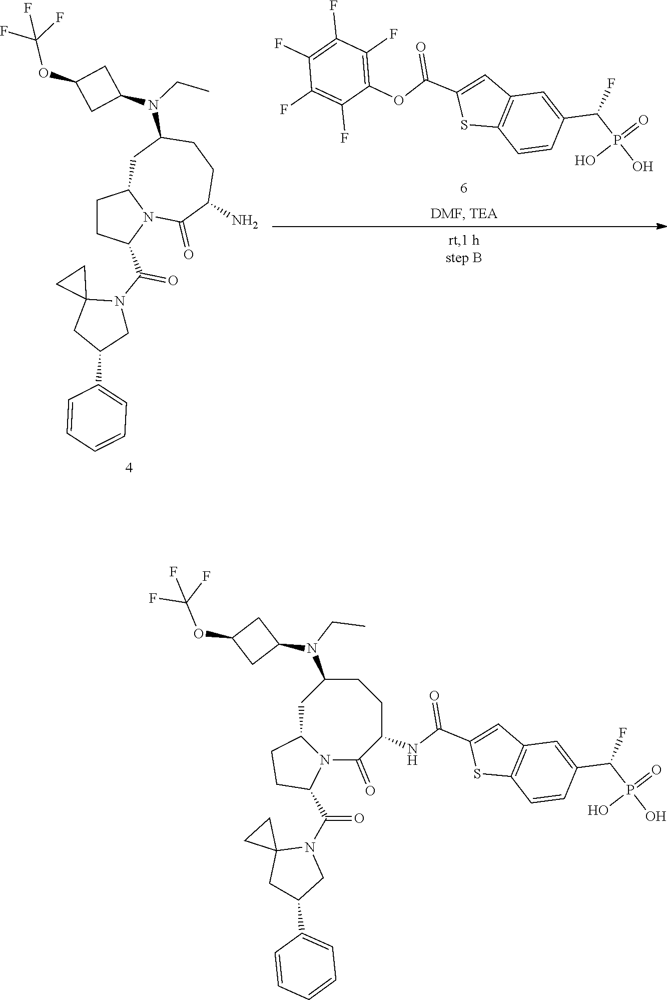 [SMILES: CCN([C@H]1CC[C@H](N)C(=O)N2[C@H](CC[C@H]2C(=O)N2C[C@H](c3ccccc3)CC23CC3)C1)[C@H]1C[C@@H](OC(F)(F)F)C1 ; O=C(Oc1c(F)c(F)c(F)c(F)c1F)c1cc2cc([C@H](F)P(=O)(O)O)ccc2s1 ; CCN([C@H]1CC[C@H](NC(=O)c2cc3cc([C@H](F)P(=O)(O)O)ccc3s2)C(=O)N2[C@H](CC[C@H]2C(=O)N2C[C@H](c3ccccc3)CC23CC3)C1)[C@H]1C[C@@H](OC(F)(F)F)C1]

To a solution of (3S,6S,9S,10aR)-6-amino-9-(ethyl((1s,3R)-3-(trifluoromethoxy)cyclobutyl)amino)-3-((S)-6-phenyl-4-azaspiro[2.4]heptane-4-carbonyl)octahydropyrrolo[1,2-a]azocin-5(1H)-one (4, 30 mg, TFA salt) in DMF (2 mL) was added TEA (13.7 mg, 0.135 mmol, 3 eq.), (R)-(fluoro(2-((perfluorophenoxy)carbonyl)benzo[b]thiophen-5-yl)methyl)phosphonic acid (6, 20 mg, 0.045 mmol, 1 eq.). The resulting mixture was stirred for 1 h at room temperature. The crude product was purified by Prep-HPLC to ((R)-(2-(((3S,6S,9S,10aR)-9-(ethyl((1s,3R)-3-(trifluoromethoxy)cyclobutyl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)fluoromethyl)phosphonic acid (Compound A42, 20 mg, 0.024 mmol, 54.5%) as a white solid. LCMS (ESI): m/z=835.4 [M+H]⁺. ¹H NMR (400 MHz, CD₃OD-d₄) δ (ppm) 8.05 (d, J=9.8 Hz, 2H), 7.93 (d, J=8.5 Hz, 1H), 7.60 (d, J=8.6 Hz, 1H), 7.38-7.27 (m, 4H), 7.21 (t, J=7.3 Hz, 1H), 5.79 (dd, J=44.8, 8.0 Hz, 1H), 4.70-4.47 (m, 4H), 4.11-3.90 (m, 3H), 3.82-3.51 (m, 3H), 3.13 (s, 1H), 2.88-2.81 (m, 1H), 2.70-2.42 (m, 3H), 2.39-2.14 (m, 6H), 2.11-1.84 (m, 8H), 1.38-1.35 (m, 3H), 0.61-0.48 (m, 2H). ¹⁹F NMR (377 MHz, CD₃OD-d₄) δ (ppm) −61.29 (s), −198.66 (d, J=83.0 Hz). ³¹P NMR (162 MHz, CD₃OD-d₄) δ (ppm) 12.31 (d, J=84.2 Hz). (TFA salt).

### Synthesis of propyl ((R)-((R)-fluoro(2-(((3S,6S,9S,10aR)-9-(methyl(oxetan-3-yl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C37) and ((R)-fluoro(2-(((3S,6S,9S,10aR)-9-(methyl(oxetan-3-yl)amino)-5-oxo-3-((S)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A13)

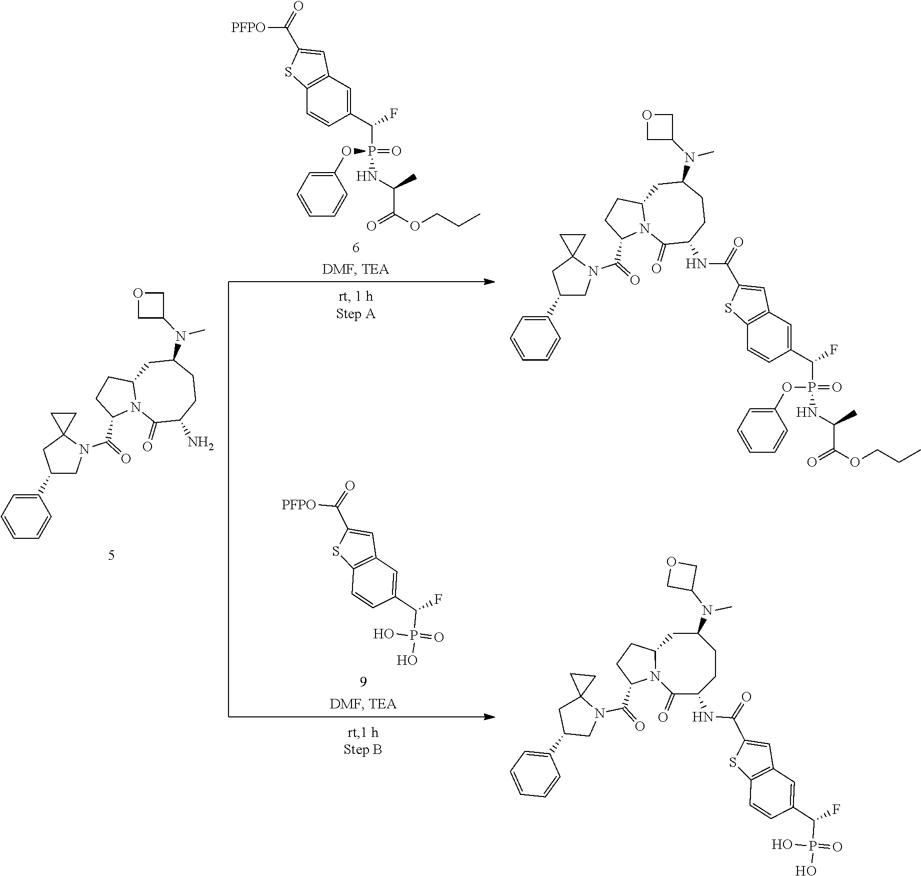

### STEP A: propyl ((R)-((R)-fluoro(2-(((3S,6S,9S,10aR)-9-(methyl(oxetan-3-yl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate

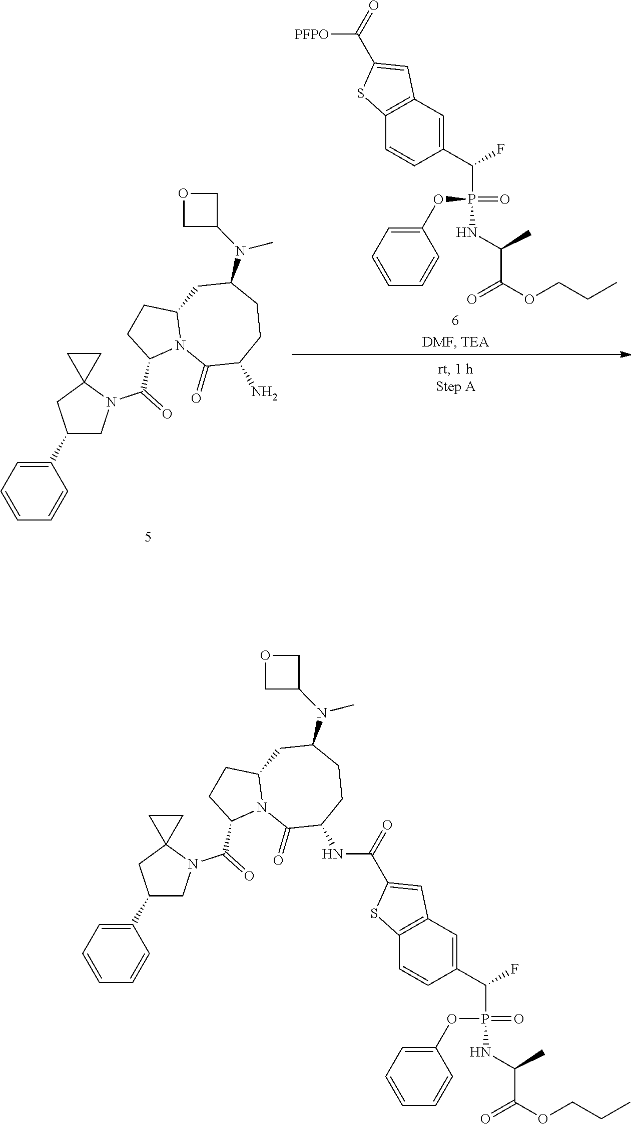

Procedure for 5 is described in the syntheses of Compound A16 and Compound C30. To a solution of (3S,6S,9S,10aR)-6-amino-9-(methyl(oxetan-3-yl)amino)-3-((R)-6-phenyl-4-azaspiro[2.4]heptane-4-carbonyl)octahydropyrrolo[1,2-a]azocin-5(1H)-one (5, 50 mg, TFA salt) in DMF (2 mL) were added TEA (25.75 mg. 0.255 mmol, 3 eq.) and perfluorophenyl 5-((R)-fluoro((R)-(((S)-1-oxo-1-propoxypropan-2-yl)amino)(phenoxy)phosphoryl)methyl)benzo[b]thiophene-2-carboxylate (6, 55 mg, 0.085 mmol, 1 eq.), and the resulting mixture was stirred at room temperature for 1 h. The crude product was purified by Prep-HPLC to afford propyl ((R)-((R)-fluoro(2-(((3S,6S,9S,10aR)-9-(methyl(oxetan-3-yl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)(phenoxy)phosphoryl)-L-alaninate (Compound C37, 46 mg, 0.049 mmol, 57.6%) as a white solid. LCMS (ESI): m/z=928.5 [M+H]⁺. ¹H NMR (400 MHz, DMSO-d₆) δ (ppm) 8.81-8.74 (m, 1H), 8.27 (s, 1H), 8.09-8.02 (m, 2H), 7.64-7.58 (m, 1H), 7.40-7.12 (m, 10H), 6.27-6.03 (m, 2H), 4.80-4.63 (m, 1H), 4.59-4.23 (m, 6H), 4.18-4.04 (m, 1H), 3.89-3.66 (m, 5H), 3.59-3.48 (m, 1H), 2.88-2.76 (m, 1H), 2.41-2.29 (m, 1H), 2.27-2.16 (m, 1H), 2.15-2.07 (m, 1H), 2.06-1.86 (m, 6H), 1.85-1.34 (m, 10H), 1.17-1.08 (m, 3H), 0.82-0.73 (m, 3H), 0.57-0.42 (m, 2H). ¹⁹F NMR (376 MHz, DMSO-d₆) δ (ppm) −194.21-194.43 (m). ³¹P NMR (162 MHz, DMSO-d₆) δ (ppm) 17.58-17.07 (m).

### Step B: ((R)-fluoro(2-(((3S,6S,9S,10aR)-9-(methyl(oxetan-3-yl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid

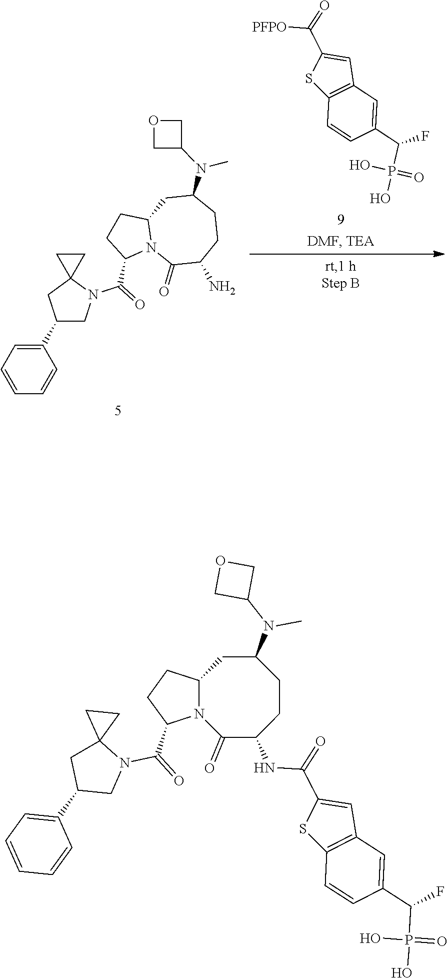

To a solution of (3S,6S,9S,10aR)-6-amino-9-(methyl(oxetan-3-yl)amino)-3-((R)-6-phenyl-4-azaspiro[2.4]heptane-4-carbonyl)octahydropyrrolo[1,2-a]azocin-5(1H)-one (5, 50 mg, TFA salt) in DMF (2 mL) were added TEA (25.75 mg. 0.255 mmol, 3 eq.) and (R)-(fluoro(2-((perfluorophenoxy)carbonyl)benzo[b]thiophen-5-yl)methyl)phosphonic acid (9, 40 mg, 0.088 mmol, 1 eq.), and the resulting mixture was stirred at room temperature for 1 h. The crude product was purified by Prep-HPLC to afford ((R)-fluoro(2-(((3S,6S,9S,10aR)-9-(methyl(oxetan-3-yl)amino)-5-oxo-3-((R)-6-phenyl-4-azaspiro[2.4]heptane-4-carbonyl)decahydropyrrolo[1,2-a]azocin-6-yl)carbamoyl)benzo[b]thiophen-5-yl)methyl)phosphonic acid (Compound A13, 36 mg, 0.048 mmol, 54.5%) as a white solid. LCMS (ESI): m/z=739.6 [M+H]⁺. ¹H NMR (400 MHz, DMSO-d₆) δ (ppm) 10.85-10.19 (m, 1H), 8.87-8.68 (m, 1H), 8.24 (s, 1H), 8.03 (d, J=8.5 Hz, 1H), 7.97 (s, 1H), 7.53 (d, J=8.6 Hz, 1H), 7.38-7.30 (m, 4H), 7.25-7.21 (m, 1H), 5.83 (dd, J=44.4, 8.0 Hz, 1H), 4.92-4.50 (m, 8H), 4.18-4.09 (m, 1H), 3.74-3.68 (m, 1H), 3.60-3.52 (m, 2H), 2.60 (s, 3H), 2.48-2.41 (m, 1H), 2.28-2.21 (m, 1H), 2.16-1.70 (m, 11H), 1.57-1.51 (m, 1H), 0.61-0.48 (m, 2H). ¹⁹F NMR (377 MHz, DMSO-d₆) δ (ppm) −194.91 (d, J=80.8 Hz). ³¹P NMR (162 MHz, DMSO-d₆) δ (ppm) 10.92 (d, J=80.9 Hz).

The following compounds in Table 1A, Table 1B, and Table 1C were prepared according to the representative procedures described above, using the appropriate starting materials and modifications.

TABLE 1A

| No. | Name | Structure | NMR and LCMS |
|---|---|---|---|
| A1 | ((S)-fluoro(2- (((3S,6S,9S, 10aR)-9-(((1s, 3R)-3-fluoro- cyclobutyl) (methyl) amino)-5-oxo- 3-((S)-6- phenyl-4- azaspiro[2.4] heptane-4- carbonyl) decahydro- pyrrolo[1,2- a]azocin-6- yl)carbamoyl) benzo[b] thiophen-5- yl)methyl) phosphonic acid | 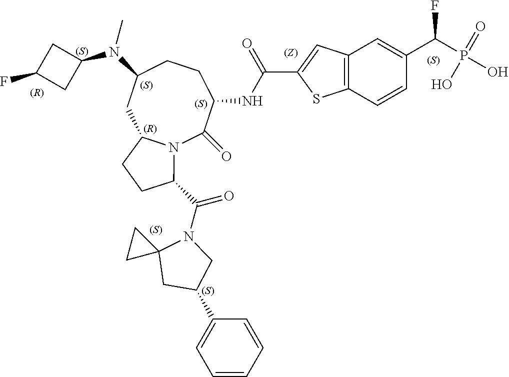 | ¹H NMR (400 MHz, MeOD-d4) δ (ppm) 8.12-8.02 (m, 2H), 7.89 (d, J = 8.4 Hz, 1H), 7.61 (d, J = 8.3 Hz, 1H), 7.33-7.17 (m, 5H), 5.67 (dd, J = 45.2, 8.3 Hz, 1H), 4.78-4.52 (m, 3H), 4.44-4.36 (m, 1H), 4.01-3.75 (m, 3H), 3.56-3.46 (m, 1H), 3.01-2.84 (m, 1H), 2.77-1.55 (m, 21H), 0.55-0.39 (m, 2H); 755.2 [M + H]⁺. |
| A2 | ((R)-fluoro(2- (((3S,6S,9S, 10aR)-9- (methyl((1s, 3R)-3- (trifluoromethyl) cyclobutyl) amino)-5-oxo-3- ((S)-6-phenyl-4- azaspiro[2.4] heptane-4- carbonyl) decahydro- pyrrolo[1,2-a] azocin-6-yl) carbamoyl) benzo[b] thiophen-5- yl)methyl) phosphonic acid | 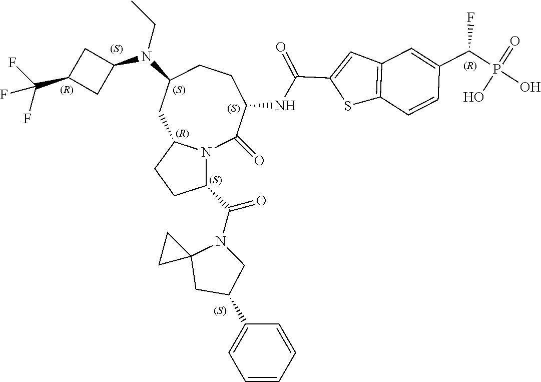 | ¹H NMR (400 MHz, DMSO-d6) δ (ppm) 8.98-8.82 (m, 1H), 8.77-8.68 (m, 1H), 8.27-8.19 (m, 1H), 8.08-7.92 (m, 2H), 7.65-7.25 (m, 2H), 6.85-6.65 (m, 2H), 5.65 (dd, J = 44.8, 8.1 Hz, 1H), 5.14- 4.88 (m, 3H), 4.40- 4.28 (m, 1H), 3.58- 3.46 (m, 1H), 3.41- 3.29 (m, 1H), 2.32- 2.07 (m, 4H), 2.02- 1.53 (m, 9H), 1.52- 1.40 (m, 3H), 1.38- 1.14 (m, 1H), 1.08- 0.80 (m, 2H), 0.78- 0.55 (m, 2H); 805.3 [M + H]⁺. |
| A3 | ((R)-fluoro(2- (((3S,6S,9S, 10aR)-9- (methyl(4,4,4- trifluorobutyl) amino)-5-oxo- 3-((S)-6-phenyl- 4-azaspiro[2.4] heptane-4- carbonyl) decahydro- pyrrolo[1,2-a] azocin-6-yl) carbamoyl) benzo[b] thiophen-5- yl)methyl) phosphonic acid | 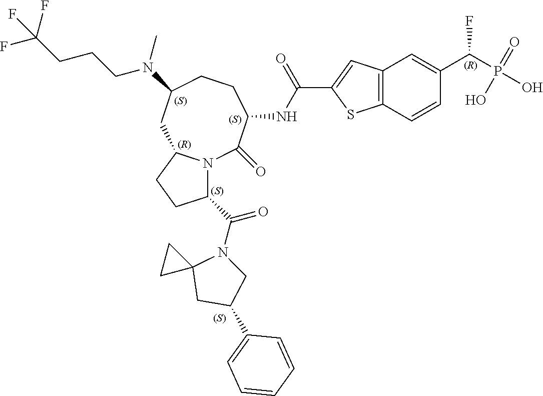 | 1H NMR (400 MHz, MeOD-d4) δ (ppm) 8.09 (s, 1H), 8.05 (s, 1H), 7.94 (d, J = 8.5 Hz, 1H), 7.70 (d, J = 8.6 Hz, 1H), 7.38-7.26 (m, 4H), 7.25-7.18 (m, 1H), 5.68 (dd, J = 45.3, 8.1 Hz, 1H), 4.64-4.47 (m, 2H), 4.38-4.21 (m, 1H), 4.07-3.80 (m, 2H), 3.77-3.63 (m, 1H), 3.58-3.47 (m, 1H), 3.14-2.94 (m, 2H), 2.73-2.60 (m, 3H), 2.50-2.36 (m, 1H), 2.20-1.77 (m, 10H), 1.91-1.78 (m, 3H), 1.76-1.50 (m, 4H), 0.60-0.36 (m, 2H); 793.2 [M + H]⁺. |
| A4 | ((R)-fluoro(2- (((3S,6S,9S, 10aR)-9- (methyl((1s,3R)- 3-(trifluoro- methoxy) cyclobutyl) amino)-5-oxo-3- ((S)-6-phenyl-4- azaspiro[2.4] heptane-4- carbonyl) decahydro- pyrrolo[1,2- a]azocin-6- yl)carbamoyl) benzo[b] thiophen-5- yl)methyl) phosphonic acid | 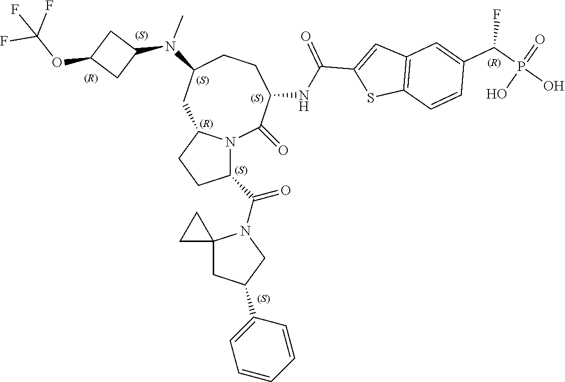 | 1H NMR (400 MHz, MeOD-d4) δ (ppm) 8.09-8.02 (m, 2H), 7.91 (d, J = 8.5 Hz, 1H), 7.69 (d, J = 8.5 Hz, 1H), 7.38-7.27 (m, 4H), 7.25-7.18 (m, 1H), 5.68 (dd, J = 45.3, 8.3 Hz, 1H), 4.71- 4.64 (m, 1H), 4.63- 4.49 (m, 2H), 4.45- 4.35 (m, 1H), 4.08- 3.85 (m, 2H), 3.70- 3.47 (m, 2H), 3.28- 3.12 (m, 1H), 2.98- 2.83 (m, 1H), 2.79- 2.64 (m, 1H), 2.63- 2.51 (m, 1H), 2.47- 1.59 (m, 18H), 0.60- 0.40 (m, 2H); 821.2 [M + H]⁺. |
| A5 | ((S)-fluoro(2- (((3S,6S,9S, 10aR)-9- (methyl((1s,3R)- 3- (trifluoro- methoxy)cyclo- butyl)amino)-5- oxo-3-((S)-6- phenyl-4- azaspiro[2.4] heptane-4- carbonyl) decahydro- pyrrolo[1,2-a] azocin-6- yl)carbamoyl) benzo[b] thiophen-5- yl)methyl) phosphonic acid | 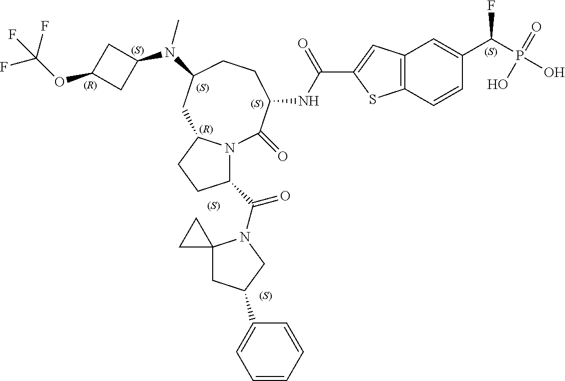 | 1H NMR (400 MHz, MeOD-d4) δ (ppm) 8.12-8.03 (m, 1H), 7.89 (d, J = 8.4 Hz, 1H), 7.60 (d, J = 8.4 Hz, 1H), 7.36-7.23 (m, 4H), 7.22-7.16 (m, 1H), 5.69 (dd, J = 45.2, 8.4 Hz, 1H), 4.71- 4.31 (m, 4H), 4.14- 3.78 (m, 3H), 3.56- 3.43 (m, 2H), 2.82- 2.67 (m, 2H), 2.65- 1.52 (m, 19H), 0.56- 0.38 (m, 2H); 821.3 [M + H]⁺. |
| A6 | ((R)-(2- (((3S,6S,9S, 10aR)-3-((6S, 7R)-7-cyano-6- phenyl-4- azaspiro[2.4] heptane-4- carbonyl)-9- morpholino-5- oxodecahydro- pyrrolo[1,2-a] azocin-6- yl)carbamoyl) benzo[b] thiophen-5- yl)fluoromethyl) phosphonic acid | 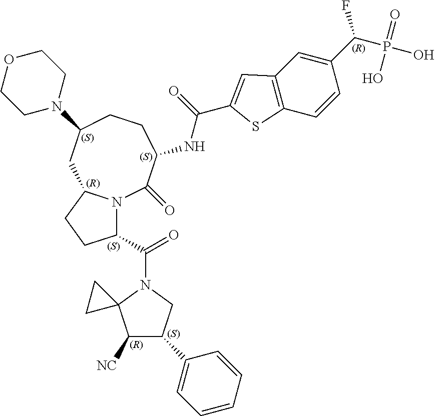 | ¹H NMR (400 MHz, MeOD) δ 8.08 (s, 1H), 8.01 (s, 1H), 7.94-7.92 (m, 1H), 7.45-7.27 (m, 5H), 5.77-5.58 (m, 1H), 4.62-4.57 (m, 1H), 4.51-4.32 (m, 2H), 4.15-4.05 (m, 1H), 3.87-3.75 (m, 5H), 3.74-3.67 (m, 2H), 3.10 3.00 (m, 1H), 2.90-2.80 (m, 4H), 2.41-2.24 (m, 2H), 2.22-2.09 (m, 1H), 2.04-1.90 (m, 4H), 1.87-1.67 (m, 3H), 1.53-1.43 (m, 1H), 1.35-1.25 (m, 1H), 0.99-0.89 (m, 1H), 0.75-0.64 (m, 1H); 764.1 [M + H]⁺. |
| A7 | ((R)-fluoro(2- (((3S,6S,9S, 10aR)-9- morpholino-5- oxo-3-((R)-6- (pyridin-3-yl)- 4-azaspiro[2.4] heptane-4- carbonyl) decahydro- pyrrolo[1,2-a] azocin-6- yl)carbamoyl) benzo[b] thiophen-5- yl)methyl) phosphonic acid | 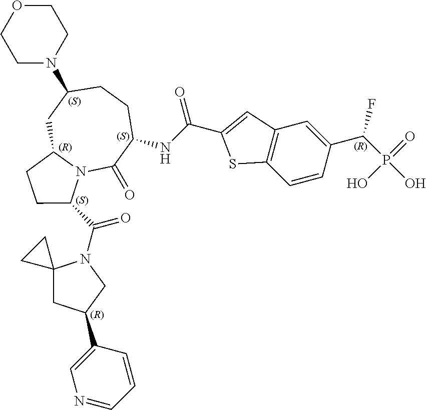 | 1H NMR (400 MHz, DMSO-d6) δ (ppm) 8.68-8.57 (m, 1H), 8.57-8.51 (m, 1H), 8.45-8.38 (m, 1H), 8.21 (s, 1H), 8.04-7.91 (m, 2H), 7.81-7.71 (m, 1H), 7.61-7.52 (m, 1H), 7.35-7.26 (m, 1H), 5.58 (dd, J = 45.4, 8.1 Hz, 1H), 4.49-4.40 (m, 2H), 4.17-4.04 (m, 3H), 3.78-3.73 (m, 3H), 3.58-3.50 (m, 2H), 3.21-3.13 (m, 1H), 2.91-2.68 (m, 4H), 2.21-1.56 (m, 12H), 1.49-1.17 (m, 2H), 0.54-0.31 (m, 2H); 740.4 [M + H]⁺. |
| A8 | ((R)-(2- (((3S,6S,9S, 10aR)-3-((R)-6- (4-(dimethyl- amino)pyridin- 3-yl)-4- azaspiro[2.4] heptane-4- carbonyl)-9- morpholino-5- oxodecahydro- pyrrolo[1,2-a] azocin-6- yl)carbamoyl) benzo[b] thiophen-5- yl)fluoromethyl) phosphonic acid | 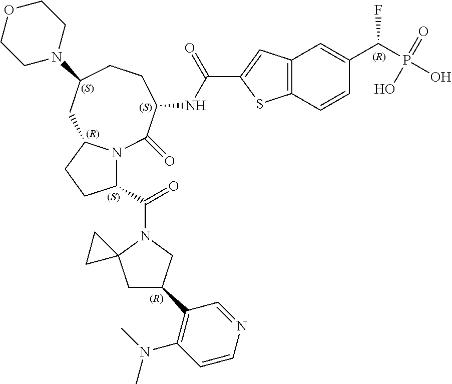 | 1H NMR (400 MHz, DMSO-d6) δ (ppm) 8.71 (d, J = 7.3 Hz, 1H), 8.37 (s, 1H), 8.27-8.21 (m, 2H), 8.03 (d, J = 8.5 Hz, 1H), 7.98 (s, 1H), 7.54 (d, J = 8.6 Hz, 1H), 7.11 (d, J = 7.1 Hz, 1H), 5.92- 5.60 (m, 1H), 4.84- 4.77 (m, 1H), 4.59- 4.51 (m, 2H), 4.13- 3.99 (m, 4H), 3.93- 3.85 (m, 2H), 3.82- 3.62 (m, 6H), 3.17 (s, 6H), 2.46-2.38 (m, 1H), 2.28-2.16 (m, 2H), 2.15-1.98 (m, 5H), 1.96-1.75 (m, 5H), 1.74-1.66 (m, 1H), 0.58-0.46 (m, 2H); 783.5 [M + H]⁺. |
| A9 | ((R)-fluoro(2- (((3S,6S,9S, 10aR)-9- morpholino-5- oxo-3-((S)-6- (pyridin-3-yl)-4- azaspiro[2.4] heptane-4- carbonyl) decahydro- pyrrolo[1,2-a] azocin-6- yl)carbamoyl) benzo[b] thiophen-5-yl) methyl) phosphonic acid | 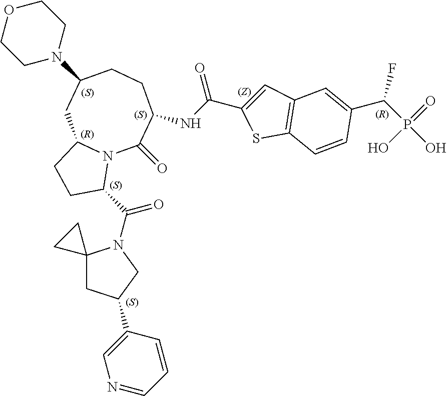 | 1H NMR (400 MHz, DMSO-d6) δ (ppm) 8.68-8.59 (m, 1H), 8.59-8.53 (m, 1H), 8.47-8.39 (m, 1H), 8.25-8.11 (m, 1H), 8.05-7.87 (m, 2H), 7.83-7.68 (m, 1H), 7.62-7.51 (m, 1H), 7.41-7.28 (m, 1H), 5.58 (dd, J = 45.2, 8.1 Hz, 1H), 4.55-4.48 (m, 1H), 4.43-4.28 (m, 2H), 4.15-4.07 (m, 1H), 3.81 (s, 4H), 3.60- 3.47 (m, 3H), 3.25- 3.16 (m, 1H), 2.82 (s, 3H), 2.40-2.21 (m, 2H), 2.13-1.13 (m, 12H), 0.46 (s, 2H); 740.5 [M + H]⁺. |
| A10 | ((S)-fluoro(2- (((3S,6S,9S, 10aR)-9- morpholino-5- oxo-3-((R)-6- (pyridin-3-yl)-4- azaspiro[2.4] heptane-4- carbonyl)deca- hydropyrrolo [1,2-a]azocin-6- yl)carbamoyl) benzo[b] thiophen-5-yl) methyl) phosphonic acid | 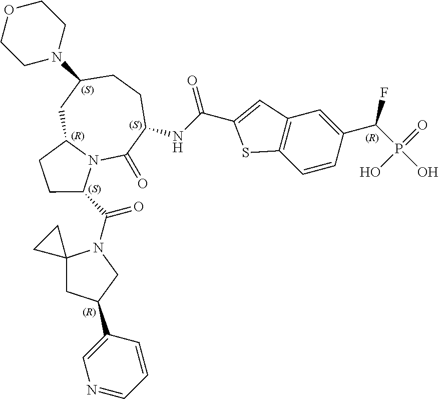 | 1H NMR (400 MHz, DMSO-d6) δ (ppm) 8.68-8.34 (m, 3H), 8.25-7.87 (m, 3H), 7.78-7.23 (m, 3H), 5.58 (dd, J = 45.4, 8.0 Hz, 1H), 4.55-4.38 (m, 2H), 4.22-3.99 (m, 3H), 3.88-3.67 (m, 4H), 3.56-3.46 (m, 1H), 3.28-3.15 (m, 1H), 2.94-2.63 (m, 4H), 2.42-1.13 (m, 14H), 0.51-0.23 (m, 2H); 740.4 [M + H]⁺. |
| A11 | ((R)-(2- (((3S,6S,9S, 10aR)-3-((6S, 7R)-7-cyano-6- (pyridin-3-yl)-4- azaspiro[2.4] heptane-4- carbonyl)-9- morpholino-5- oxodecahydro- pyrrolo[1,2-a] azocin-6- yl)carbamoyl) benzo[b] thiophen-5- yl)fluoromethyl) phosphonic acid | 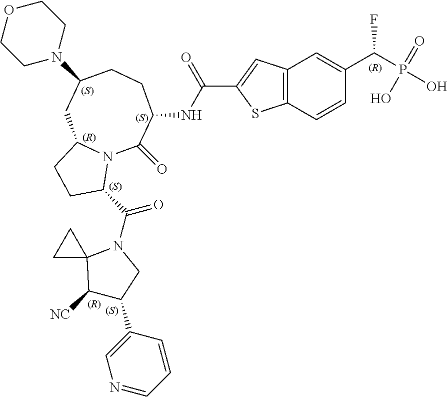 | 1H NMR (400 MHz, DMSO-d6) δ (ppm) 8.69-8.49 (m, 3H), 8.24-8.11 (m, 1H), 8.01-7.83 (m, 3H), 7.59-7.54 (m, 1H), 7.44-7.38 (m, 1H), 5.58 (dd, J = 45.2, 8.0 Hz, 1H), 4.56-4.25 (m, 3H), 4.15-4.00 (m, 2H), 3.83-3.67 (m, 6H), 3.11-3.01 (m, 1H), 2.96-2.63 (m, 4H), 2.37-2.13 (m, 2H), 2.02-1.54 (m, 8H), 1.52-1.12 (m, 2H), 0.85-0.58 (m, 2H); 765.3 [M + H]⁺. |
| A12 | ((S)-(2- (((3S,6S,9S, 10aR)-3-((R)-6- (4-(dimethyl- amino)pyridin- 3-yl)-4- azaspiro[2.4] heptane-4- carbonyl)-9- morpholino-5- oxodecahydro- pyrrolo[1,2-a] azocin-6- yl)carbamoyl) benzo[b] thiophen-5- yl)fluoromethyl) phosphonic acid | 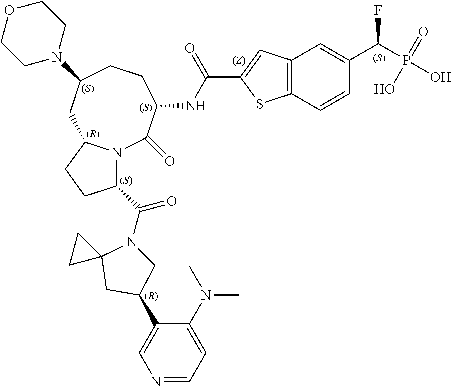 | 1H NMR (400 MHz, DMSO-d6) δ (ppm) 9.46-8.51 (m, 1H), 8.46-7.66 (m, 4H), 7.62-7.25 (m, 1H), 7.22-6.54 (m, 1H), 5.85-5.44 (m, 1H), 5.05-4.58 (m, 1H), 4.51-4.25 (m, 2H), 4.16-3.79 (m, 4H), 3.53-3.11 (m, 8H), 2.76-2.64 (m, 6H), 2.38-2.13 (m, 3H), 2.12-1.83 (m, 7H), 1.78-1.55 (m, 4H), 0.55-0.29 (m, 2H); 783.4 [M + H]⁺. |
| A13 | ((R)-fluoro(2- (((3S,6S,9S, 10aR)-9- (methyl(oxetan- 3-yl)amino)-5- oxo-3-((S)-6- phenyl-4- azaspiro[2.4] heptane-4- carbonyl) decahydro- pyrrolo[1,2-a] azocin-6- yl)carbamoyl) benzo[b] thiophen-5-yl) methyl) phosphonic acid | 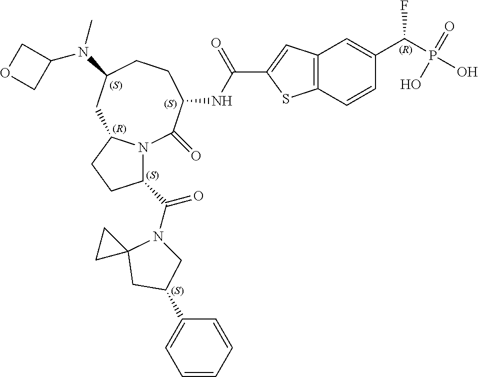 | 1H NMR (400 MHz, DMSO-d6) δ (ppm) 10.38 (s, 1H), 8.87-8.68 (m, 1H), 8.24 (s, 1H), 8.03 (d, J = 8.5 Hz, 1H), 7.97 (s, 1H), 7.53 (d, J = 8.6 Hz, 1H), 7.38-7.30 (m, 4H), 7.25-7.21 (m, 1H), 5.83 (dd, J = 44.4, 8.0 Hz, 1H), 4.92- 4.50 (m, 8H), 4.18- 4.09 (m, 1H), 3.74- 3.68 (m, 1H), 3.60- 3.52 (m, 2H), 2.60 (s, 3H), 2.48-2.41 (m, 1H), 2.28-2.21 (m, 1H), 2.16-1.70 (m, 11H), 1.57- 1.51 (m, 1H), 0.61- 0.48 (m, 2H); 739.6 [M + H]⁺. |
| A14 | ((R)-fluoro(2- (((3S,6S,9S, 10aR)-9- (methyl(oxetan- 3-ylmethyl) amino)-5-oxo-3- ((S)-6-phenyl- 4-azaspiro[2.4] heptane-4- carbonyl) decahydro- pyrrolo[1,2-a] azocin-6-yl) carbamoyl) benzo[b] thiophen-5-yl) methyl) phosphonic acid | 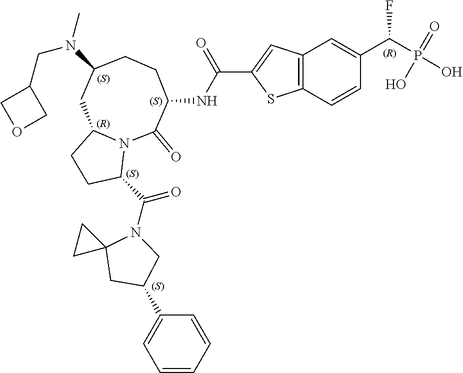 | 1H NMR (400 MHz, DMSO-d6) δ (ppm) 9.73-9.33 (m, 1H), 8.84-8.68 (m, 1H), 8.29-8.16 (m, 1H), 8.06-7.90 (m, 2H), 7.58-7.45 (m, 1H), 7.39-7.19 (m, 5H), 5.79 (dd, J = 44.4, 7.9 Hz, 1H), 4.83-4.76 (m, 1H), 4.69-4.66 (m, 1H), 4.58-4.50 (m, 2H), 4.41-4.30 (m, 2H), 4.15-4.10 (m, 1H), 3.79-3.66 (m, 3H), 3.55-3.44 (m, 3H), 3.12-3.00 (m, 1H), 2.74-2.53 (m, 3H), 2.32-1.46 (m, 14H), 0.61-0.40 (m, 2H); 753.5 [M + H]⁺. |
| A15 | ((R)-fluoro(2- (((3S,6S,9S, 10aR)-9- (methyl(((S)- oxetan-2-yl) methyl)amino)- 5-oxo-3-((S)-6- phenyl-4- azaspiro[2.4] heptane-4- carbonyl) decahydro- pyrrolo[1,2-a] azocin-6-yl) carbamoyl) benzo[b] thiophen-5-yl) methyl) phosphonic acid | 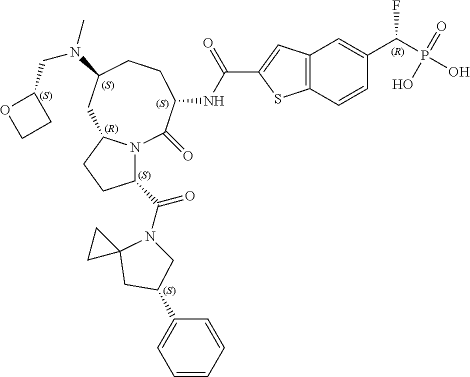 | 1H NMR (400 MHz, DMSO-d6) δ (ppm) 9.61-9.30 (m, 1H), 8.86-8.69 (m, 1H), 8.29-8.22 (m, 1H), 8.06-7.93 (m, 2H), 7.53 (d, J = 8.5 Hz, 1H), 7.40- 7.29 (m, 4H), 7.23 (t, J = 7.0 Hz, 1H), 5.81 (dd, J = 44.4, 8.0 Hz, 1H), 5.21- 5.06 (m, 1H), 4.84- 4.75 (m, 1H), 4.62- 4.51 (m, 3H), 4.15- 4.09 (m, 1H), 3.78- 3.66 (m, 2H), 3.60- 3.48 (m, 2H), 3.38- 3.16 (m, 1H), 2.83- 2.64 (m, 4H), 2.49- 1.70 (m, 15H), 1.63- 1.53 (m, 1H), 0.57- 0.43 (m, 2H); 753.3 [M + H]⁺. |
| A16 | ((S)-fluoro(2- (((3S,6S,9S, 10aR)-9- (methyl(oxetan- 3-yl)amino)-5- oxo-3-((S)-6- phenyl-4- azaspiro[2.4] heptane-4- carbonyl) decahydro- pyrrolo[1,2- a]azocin-6- yl)carbamoyl) benzo[b] thiophen-5- yl)methyl) phosphonic acid | 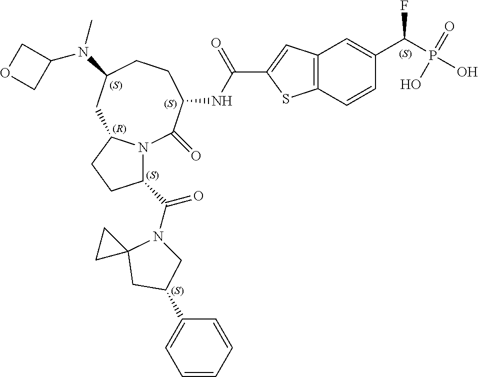 | 1H NMR (400 MHz, DMSO-d6) δ (ppm) 8.84-8.68 (m, 1H), 8.24 (s, 1H), 8.03-7.84 (m, 2H), 7.57-7.13 (m, 6H), 5.59 (dd, J = 44.9, 8.4 Hz, 1H), 4.81-4.42 (m, 6H), 4.39-4.27 (m, 1H), 4.24-4.00 (m, 2H), 3.77-3.65 (m, 1H), 3.56-3.46 (m, 1H), 3.24-3.03 (m, 1H), 2.44-1.44 (m, 17H), 0.55-0.36 (m, 2H); 739.7 [M + H]⁺. |
| A17 | ((R)-fluoro(2- (((3S,6S,9S, 10aR)-9- (methyl(((R)- oxetan-2- yl)methyl) amino)-5-oxo- 3-((S)-6-phenyl- 4-azaspiro[2.4] heptane-4- carbonyl) decahydro- pyrrolo[1,2-a] azocin-6- yl)carbamoyl) benzo[b] thiophen-5- yl)methyl) phosphonic acid | 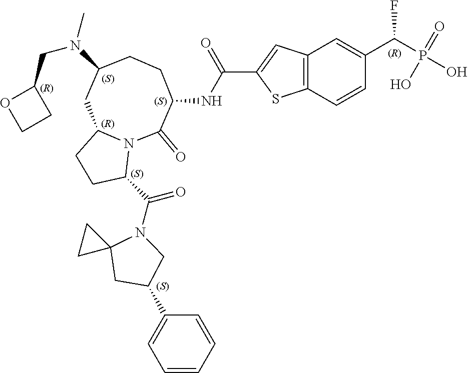 | 1H NMR (400 MHz, DMSO-d6) δ (ppm) 9.59-9.23 (m, 1H), 8.86-8.63 (m, 1H), 8.25 (s, 1H), 8.04-7.89 (m, 2H), 7.52 (d, J = 8.5 Hz, 1H), 7.39-7.20 (m, 5H), 5.78 (dd, J = 44.4, 7.9 Hz, 1H), 5.26-5.04 (m, 1H), 4.84-4.73 (m, 1H), 4.64-4.46 (m, 4H), 4.17-4.08 (m, 1H), 3.77-3.68 (m, 2H), 3.57-3.50 (m, 2H), 3.34-3.22 (m, 1H), 2.82-2.64 (m, 4H), 2.48-2.15 (m, 4H), 2.13-1.71 (m, 11H), 1.63-1.55 (m, 1H), 0.60-0.43 (m, 2H); 753.2 [M + H]⁺. |
| A18 | ((R)-fluoro(2- (((3S,6S,9S, 10aR)-9-(methyl ((S)-tetrahydro- furan-3-yl) amino)-5-oxo- 3-((S)-6-phenyl- 4-azaspiro[2.4] heptane-4- carbonyl) decahydro- pyrrolo[1,2-a] azocin-6-yl) carbamoyl) benzo[b] thiophen-5-yl) methyl) phosphonic acid | 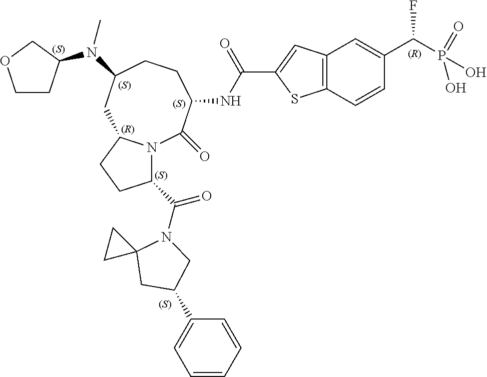 | 1H NMR (400 MHz, DMSO-d6) δ (ppm) 8.90-8.61 (m, 1H), 8.24 (s, 1H), 8.08-7.88 (m, 2H), 7.61-7.44 (m, 1H), 7.41-7.14 (m, 5H), 6.00-5.62 (m, 1H), 4.89-4.71 (m, 1H), 4.64-4.45 (m, 2H), 4.21-4.04 (m, 2H), 3.93-3.84 (m, 2H), 3.77-3.67 (m, 2H), 3.58-3.50 (m, 2H), 2.77-2.57 (m, 3H), 2.47-2.40 (m, 1H), 2.34-1.92 (m, 11H), 1.87-1.46 (m, 5H), 0.62-0.43 (m, 2H); 753.2 [M + H]⁺. |
| A19 | ((R)-fluoro(2- (((3S,6S,9S, 10aR)-9- (methyl((R)- tetrahydrofuran- 3-yl)amino)-5- oxo-3-((S)-6- phenyl-4- azaspiro[2.4] heptane-4- carbonyl) decahydro- pyrrolo[1,2-a] azocin-6-yl) carbamoyl) benzo[b] thiophen-5-yl) methyl) phosphonic acid | 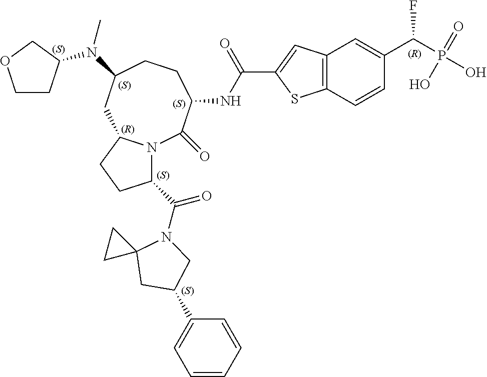 | 1H NMR (400 MHz, DMSO-d6) δ (ppm) 9.77-9.44 (m, 1H), 8.90-8.68 (m, 1H), 8.25 (s, 1H), 8.02 (d, J = 8.4 Hz, 1H), 7.97 (s, 1H), 7.53 (d, J = 8.5 Hz, 1H), 7.39-7.28 (m, 4H), 7.26-7.20 (m, 1H), 5.81 (dd, J = 44.3, 7.9 Hz, 1H), 4.87-4.75 (m, 1H), 4.61-4.45 (m, 2H), 4.18-4.10 (m, 2H), 3.99-3.93 (m, 1H), 3.85-3.80 (m, 1H), 3.77-3.63 (m, 4H), 3.58-3.52 (m, 1H), 2.74-2.62 (m, 3H), 2.48-2.40 (m, 1H), 2.35-2.17 (m, 3H), 2.17-1.91 (m, 8H), 1.86-1.69 (m, 3H), |
| | | | 1.57-1.47 (m, 1H), |
| | | | 0.65-0.41 (m, 2H); |
| | | | 753.2 |
| | | | [M + H]⁺. |
| A20 | ((R)-(2- (((3S,6S,9S, 10aR)-9-((3,3- difluoropropyl) (methyl)amino)- 5-oxo-3-((S)-6- phenyl-4- azaspiro[2.4] heptane-4- carbonyl)deca- hydropyrrolo [1,2-a]azocin-6- yl)carbamoyl) benzo[b] thiophen-5-yl) fluoromethyl) phosphonic acid | 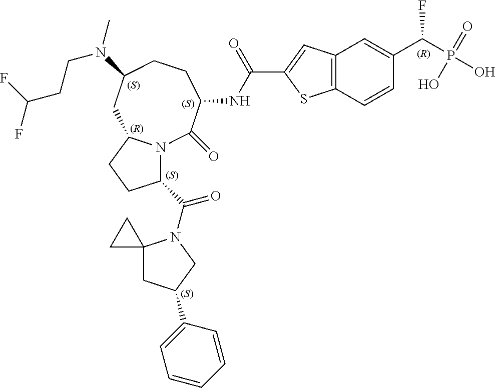 | 1H NMR (400 MHz, DMSO-d6) δ (ppm) 8.87-8.67 (m, 1H), 8.24 (s, 1H), 8.03 (d, J = 8.4 Hz, 1H), 7.98 (s, 1H), 7.53 (d, J = 8.4 Hz, 1H), 7.38-7.29 (m, 4H), 7.26-7.19 (m, 1H), 6.40-6.03 (m, 1H), 5.83 (dd, J = 44.3, 8.0 Hz, 1H), 4.81 (s, 1H), 4.60- 4.50 (m, 2H), 4.17- 4.09 (m, 1H), 3.85- 3.70 (m, 2H), 3.57- 3.51 (m, 1H), 3.35- 3.19 (m, 2H), 2.80- 2.69 (m, 3H), 2.49- 2.42 (m, 1H), 2.39- 1.70 (m, 14H), 1.56 (s, 1H), 0.59-0.42 (m, 2H); 761.5 [M + H]⁺. |
| A21 | ((R)-fluoro(2- (((3S,6S,9S, 10aR)-9-(((1s, 3R)-3-fluoro- cyclobutyl) (methyl)amino)- 5-oxo-3-((S)-6- phenyl-4- azaspiro[2.4] heptane-4- carbonyl)deca- hydropyrrolo [1,2-a]azocin-6- yl)carbamoyl) benzo[b] thiophen-5-yl) methyl) phosphonic acid | 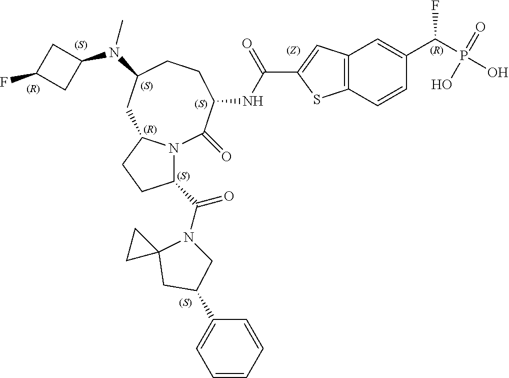 | 1H NMR (400 MHz, MeOD-d4) δ (ppm) 8.08-8.01 (m, 2H), 7.88 (d, J = 8.4 Hz, 1H), 7.66 (d, J = 8.4 Hz, 1H), 7.37-7.26 (m, 4H), 7.24-7.17 (m, 1H), 5.65 (dd, J = 45.2, 8.4 Hz, 1H), 4.79- 4.68 (m, 2H), 4.66- 4.58 (m, 1H), 4.52- 4.40 (m, 1H), 4.07- 3.99 (m, 1H), 3.96- 3.89 (m, 1H), 3.86- 3.73 (m, 1H), 3.61- 3.49 (m, 1H), 3.00- 2.86 (m, 1H), 2.81- 2.68 (m, 1H), 2.65- 2.06 (m, 11H), 2.03- 1.60 (m, 9H), 0.59- 0.44 (m, 2H); [M + H]⁺. |
| A22 | ((R)-fluoro(2- (((3S,6S,9S, 10aR)-9-(((1r, 3S)-3-fluoro- cyclobutyl) (methyl)amino)- 5-oxo-3-((S)-6- phenyl-4- azaspiro[2.4] heptane-4- carbonyl)deca- hydropyrrolo [1,2-a]azocin-6- yl)carbamoyl) benzo[b] thiophen-5-yl) methyl) phosphonic acid | 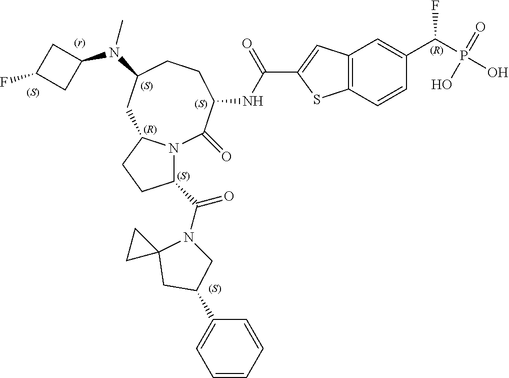 | 1H NMR (400 MHz, DMSO-d6) δ (ppm) 8.90-8.56 (m, 1H), 8.23 (s, 1H), 8.04-7.87 (m, 2H), 7.67-7.43 (m, 1H), 7.37-7.19 (m, 5H), 5.77-5.57 (m, 1H), 5.36-5.09 (m, 1H), 4.55-4.41 (m, 2H), 4.15-4.02 (m, 2H), 3.77-3.66 (m, 2H), 3.56-3.45 (m, 2H), 2.96-2.60 (m, 3H), 2.46-2.13 (m, 5H), 2.12-1.42 (m, 13H), 0.63-0.40 (m, 2H); 755.3 [M + H]⁺. |
| A23 | 2,2,2- trifluoroacetic acid compound with ((R)-(2- (((3S,6S,9S, 10aR)-9-((3,3- difluorocyclo- butyl)(methyl) amino)-5-oxo- 3-((S)-6-phenyl- 4-azaspiro[2.4] heptane-4- carbonyl)deca- hydropyrrolo [1,2-a]azocin-6- yl)carbamoyl) benzo[b] thiophen-5- yl)fluoromethyl) phosphonic acid | 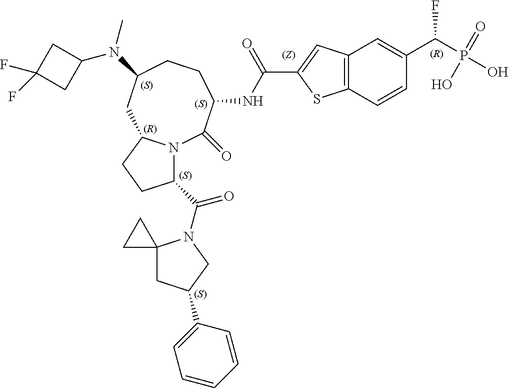 | 1H NMR (400 MHz, DMSO-d6) δ (ppm) 10.48-9.56 (m, 1H), 8.78 (s, 1H), 8.24 (s, 1H), 8.05-7.95 (m, 2H), 7.53 (d, J = 8.5 Hz, 1H), 7.38-7.29 (m, 4H), 7.23 (t, J = 7.0 Hz, 1H), 5.76 (dd, J = 44.6, 7.0 Hz, 1H), 4.84-4.74 (m, 1H), 4.60-4.45 (m, 2H), 4.18-4.10 (m, 1H), 3.73-3.71 (m, 5H), 3.02-2.89 (m, 2H), 2.63 (s, 3H), 2.48- 2.43 (m, 1H), 2.35- 2.16 (m, 2H), 2.13- 1.75 (m, 11H), 1.57- 1.51 (m, 1H), 0.61- 0.48 (m, 2H); 773.3 [M + H]⁺. |
| A24 | ((S)-fluoro(2- (((3S,6S,9S, 10aR)-9-(methyl (((S)-oxetan-2- yl)methyl) amino)-5-oxo- 3-((S)-6- phenyl-4- azaspiro[2.4] heptane-4- carbonyl)deca- hydropyrrolo [1,2-a]azocin- 6-yl)carbamoyl) benzo[b] thiophen-5-yl) methyl) phosphonic acid | 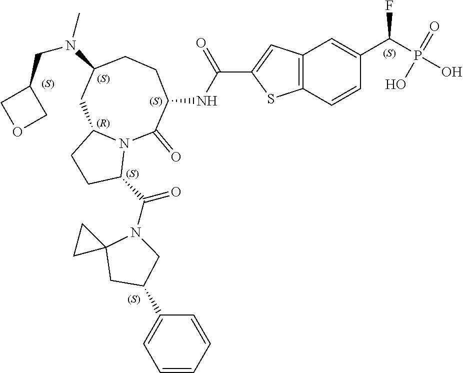 | 1H NMR (400 MHz, DMSO-d6) δ (ppm) 9.67-9.28 (m, 1H), 8.86-8.70 (m, 1H), 8.25 (s, 1H), 8.06-7.93 (m, 2H), 7.52 (d, J = 8.5 Hz, 1H), 7.38-7.27 (m, 4H), 7.26-7.19 (m, 1H), 5.78 (dd, J = 44.4, 8.0 Hz, 1H), 5.26-5.04 (m, 1H), 4.82-4.74 (m, 1H), 4.58-4.45 (m, 3H), 4.19-4.05 (m, 1H), 3.78-3.67 (m, 2H), 3.59-3.49 (m, 2H), 3.38-3.17 (m, 1H), 2.84-2.61 (m, 4H), 2.47-2.18 (m, 3H), 2.17-1.42 (m, 13H), 0.58-0.45 (m, 2H); 753.3 [M + H]⁺. |
| A25 | ((S)-(2- (((3S,6S,9S, 10aR)-9-((3,3- difluorocyclo- butyl)(methyl) amino)-5-oxo- 3-((S)-6-phenyl- 4-azaspiro[2.4] heptane-4- carbonyl)deca- hydropyrrolo [1,2-a]azocin- 6-yl)carbamoyl) benzo[b] thiophen-5-yl) fluoromethyl) phosphonic acid | 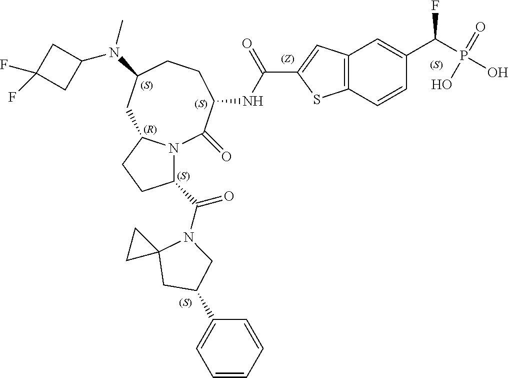 | 1H NMR (400 MHz, DMSO-d6) δ (ppm) 10.06 (d, J = 176.4 Hz, 1H), 8.77 (s, 1H), 8.24 (s, 1H), 8.06-7.94 (m, 2H), 7.53 (d, J = 8.5 Hz, 1H), 7.38-7.29 (m, 4H), 7.27-7.20 (m, 1H), 5.81 (dd, J = 44.3, 8.0 Hz, 1H), 4.85-4.75 (m, 1H), 4.58-4.46 (m, 2H), 4.14 (s, 1H), 3.82- 3.68 (m, 2H), 3.64- 3.49 (m, 2H), 3.15- 2.83 (m, 4H), 2.62 (s, 3H), 2.49-2.41 (m, 1H), 2.27-2.21 (m, 1H), 2.12-1.62 (m, 11H), 1.58-1.49 (m, 1H), 0.60-0.47 (m, 2H); 773.2 [M + H]⁺. |
| A26 | ((S)-fluoro(2- (((3S,6S,9S, 10aR)-9-(methyl ((R)-tetrahydro- furan-3-yl) amino)-5-oxo- 3-((S)-6-phenyl- 4-azaspiro[2.4] heptane-4- carbonyl)deca- hydropyrrolo [1,2-a]azocin-6- yl)carbamoyl) benzo[b] thiophen-5- yl)methyl) phosphonic acid | 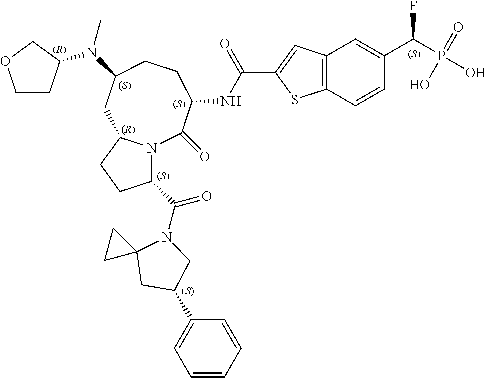 | 1H NMR (400 MHz, DMSO-d6) δ (ppm) 8.72 (s, 1H), 8.22 (s, 1H), 8.03- 7.85 (m, 2H), 7.57- 7.43 (m, 1H), 7.38- 7.25 (m, 4H), 7.23- 7.17 (m, 1H), 5.67- 5.42 (m, 1H), 4.63- 4.19 (m, 3H), 4.13- 4.04 (m, 1H), 3.93- 3.74 (m, 4H), 3.71- 3.62 (m, 3H), 2.43- 1.57 (m, 19H), 1.54- 1.48 (m, 1H), 0.56- 0.37 (m, 2H); 753.3 [M + H]⁺. |
| A27 | ((R)-fluoro(2- (((3S,6S,9S, 10aR)-3-((S)-6- (3-fluoro- phenyl)-4- azaspiro[2.4] heptane-4- carbonyl)-9- (methyl(oxetan- 3-yl)amino)-5- oxodecahydro- pyrrolo[1,2-a] azocin-6-yl) carbamoyl) benzo[b] thiophen-5-yl) methyl) phosphonic acid | 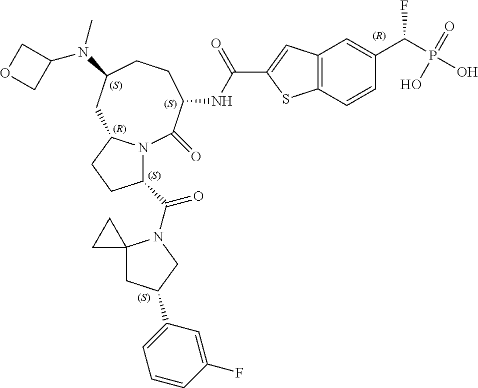 | 1H NMR (400 MHz, DMSO-d6) δ (ppm) 10.80-10.14 (m, 1H), 8.89-8.62 (m, 1H), 8.27-8.17 (m, 1H), 8.03 (s, 1H), 7.97 (s, 1H), 7.53 (d, J = 8.6 Hz, 1H), 7.40-7.33 (m, 1H), 7.25-7.18 (m, 2H), 7.09-7.03 (m, 1H), 5.83 (dd, J = 44.3, 8.0 Hz, 1H), 4.89-4.50 (m, 8H), 4.17-4.09 (m, 1H), 3.75-3.69 (m, 1H), 3.59-3.56 (m, 1H), 2.60 (s, 3H), 2.48- 2.40 (m, 1H), 2.26- 1.52 (m, 14H), 0.61- 0.40 (m, 2H); 757.4 [M + H]⁺. |
| A28 | ((S)-fluoro(2- (((3S,6S,9S, 10aR)-9-(methyl ((S)-tetrahydro- furan-3-yl) amino)-5-oxo- 3-((S)-6-phenyl- 4-azaspiro[2.4] heptane-4- carbonyl)deca- hydropyrrolo [1,2-a]azocin-6- yl)carbamoyl) benzo[b] thiophen-5-yl) methyl) phosphonic acid | 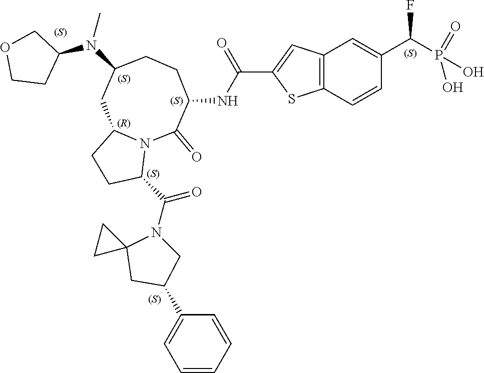 | 1H NMR (400 MHz, DMSO-d6) δ (ppm) 10.01-9.49 (m, 1H), 8.90-8.65 (m, 1H), 8.24 (s, 1H), 8.06-7.92 (m, 2H), 7.53 (d, J = 8.5 Hz, 1H), 7.39-7.27 (m, 4H), 7.26-7.20 (m, 1H), 5.80 (dd, J = 44.4, 8.0 Hz, 1H), 4.85-4.75 (m, 1H), 4.62-4.48 (m, 2H), 4.18-4.02 (m, 2H), 3.93-3.85 (m, 2H), 3.77-3.63 (m, 2H), 3.62-3.40 (m, 2H), 2.76-2.60 (m, 3H), 2.49-2.41 (m, 1H), 2.35-1.46 (m, 16H), 0.62-0.42 (m, 2H); 753.2 [M + H]⁺. |
| A29 | ((S)-(2- (((3S,5S,9S, 10aR)-9-((3,3- difluoropropyl) (methyl)amino)- 5-oxo-3-((S)-6- phenyl-4- azaspiro[2.4] heptane-4- carbonyl)deca- hydropyrrolo [1,2-a]azocin-6- yl)carbamoyl) benzo[b] thiophen-5- yl)fluoromethyl) phosphonic acid | 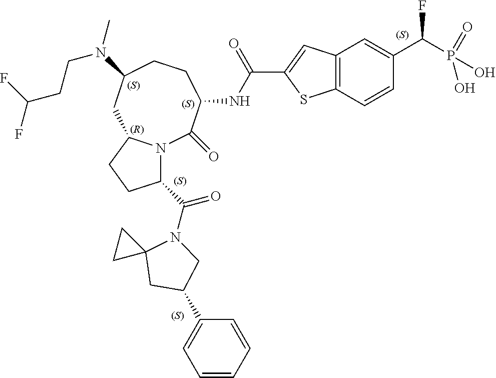 | 1H NMR (400 MHz, DMSO-d6) 1H NMR (400 MHz, DMSO-d6) δ (ppm) 8.85-8.61 (m, 1H), 8.29-8.17 (m, 1H), 8.02-7.92 (m, 2H), 7.60-7.41 (m, 1H), 7.37-7.26 (m, 4H), 7.21 (t, J = 7.0 Hz, 1H), 6.38- 5.93 (m, 1H), 5.74- 5.47 (m, 1H), 4.73- 4.32 (m, 4H), 4.13- 4.03 (m, 1H), 3.76- 3.64 (m, 1H), 3.54- 3.46 (m, 1H), 3.05 (2, 2H), 2.69-2.54 (m, 3H), 2.47-2.14 (m, 4H), 2.06-1.66 (m, 11H), 1.59- 1.51 (m, 1H), 0.51- 0.36 (m, 2H); 761.4 [M + H]⁺. |
| A30 | ((R)-fluoro(2- (((3S,6S,9S, 10aR)-9- (((1s,3R)-3- fluorocyclo- butyl)(methyl) amino)-5-oxo- 3-((R)-6- (pyridin-3-yl)- 4-azaspiro[2.4] heptane-4- carbonyl)deca- hydropyrrolo [1,2-a]azocin-6- yl)carbamoyl) benzo[b] thiophen-5- yl)methyl) phosphonic acid. | 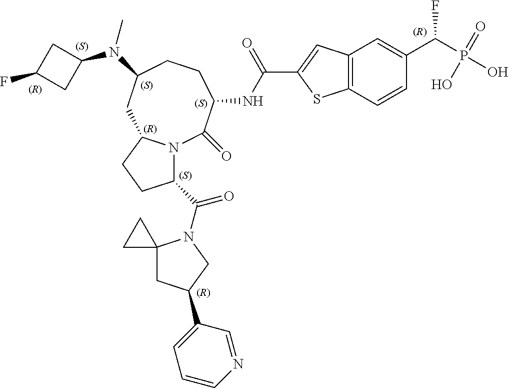 | 1H NMR (400 MHz, DMSO-d6) δ (ppm) 8.93-8.43 (m, 3H), 8.30-8.05 (m, 2H), 8.05-7.94 (m, 2H), 7.69-7.48 (m, 2H), 5.84 (dd, J = 44.3, 8.0 Hz, 1H), 5.02-4.75 (m, 2H), 4.61-4.48 (m, 2H), 4.24-4.11 (m, 1H), 3.90-3.74 (m, 2H), 3.73-3.62 (m, 1H), 3.45-3.00 (m, 1H), 2.80-2.62 (m, 2H), 2.62-2.54 (m, 3H), 2.50-2.36 (m, 3H), 2.27-1.59 (m, 13H), 0.63-0.35 (m, 2H); 756.6 [M + H]⁺. |
| A31 | ((S)-fluoro(2- (((3S,6S,9S, 10aR)-9- (methyl(((R)- oxetan-2- yl)methyl) amino)-5-oxo-3- ((S)-6-phenyl-4- azaspiro[2.4] heptane-4- carbonyl)deca- hydropyrrolo [1,2-a]azocin-6- yl)carbamoyl) benzo[b] thiophen-5- yl)methyl) phosphonic acid | 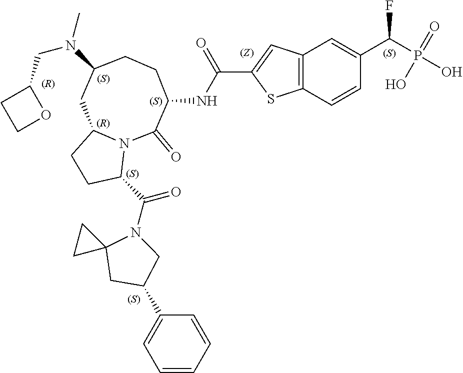 | 1H NMR (400 MHz, DMSO-d6) δ (ppm) 8.88-8.65 (m, 1H), 8.24 (s, 1H), 8.07-7.92 (m, 2H), 7.52 (d, J = 8.5 Hz, 1H), 7.38-7.16 (m, 5H), 5.77 (dd, J = 44.6, 7.7 Hz, 1H), 5.26-5.03 (m, 1H), 4.83-4.74 (m, 1H), 4.65-4.42 (m, 4H), 4.16-4.11 (m, 1H), 3.76-3.71 (m, 2H), 3.55-3.51 (m, 2H), 3.28-3.20 (m, 1H), 2.83-2.61 (m, 4H), 2.48-2.32 (m, 2H), 2.29-2.18 (m, 1H), 2.15-1.66 (m, 11H), 1.61-1.53 (m, 1H), 0.60-0.44 (m, 2H); 753.3 [M + H]⁺. |
| A32 | ((R)-(2- (((3S,6S,9S, 10aR)-9-(((S)- 3,3-difluoro- cyclopentyl) (methyl)amino)- 5-oxo-3-((S)- 6-phenyl-4- azaspiro[2.4] heptane-4- carbonyl)deca- hydropyrrolo [1,2-a]azocin-6- yl)carbamoyl) benzo[b] thiophen-5-yl) fluoromethyl) phosphonic acid | 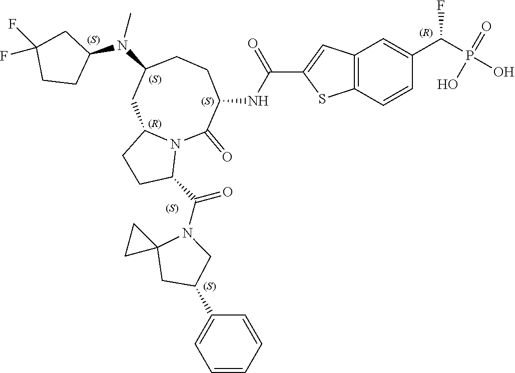 | 1H NMR (400 MHz, DMSO-d6) δ (ppm) 8.82-8.64 (m, 1H), 8.25-8.19 (m, 1H), 8.04-7.90 (m, 2H), 7.60-7.49 (m, 1H), 7.38-7.26 (m, 4H), 7.25-7.12 (m, 1H), 5.79-5.52 (m, 1H), 4.57-4.40 (m, 2H), 4.14-4.04 (m, 2H), 3.76-3.68 (m, 2H), 3.56-3.47 (m, 2H), 2.66-2.55 (m, 3H), 2.48-2.16 (m, 5H), 2.16-1.85 (m, 9H), 1.85-1.64 (m, 5H), 1.64-1.52 (m, 1H), 0.60-0.38 (m, 2H); 787.4 [M + H]⁺. |
| A33 | ((R)-(2- (((3S,6S,9S, 10aR)-9-(ethyl ((1s,3R)-3- fluorocyclo- butyl)amino)-5- oxo-3-((S)-6- phenyl-4- azaspiro[2.4] heptane-4- carbonyl)deca- hydropyrrolo [1,2-a]azocin-6- yl)carbamoyl) benzo[b] thiophen-5-yl) fluoromethyl) phosphonic acid | 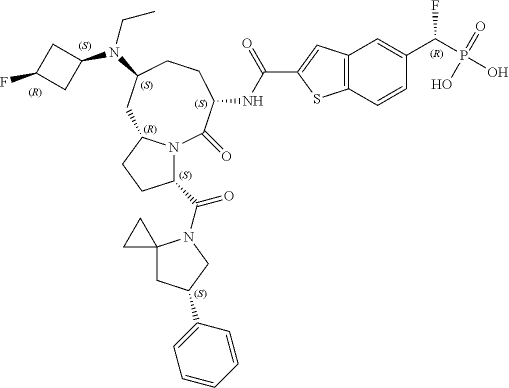 | 1H NMR (400 MHz, DMSO-d6) δ (ppm) 9.65-9.12 (m, 1H), 8.90-8.71 (m, 1H), 8.30-8.22 (m, 1H), 8.03-7.91 (m, 2H), 7.55-7.47 (m, 1H), 7.38-7.20 (m, 5H), 5.86-5.63 (m, 1H), 5.00-4.72 (m, 2H), 4.59-4.45 (m, 2H), 4.20-4.09 (m, 1H), 3.77-3.72 (m, 1H), 3.57-3.53 (m, 1H), 3.24-3.15 (m, 1H), 3.09-3.03 (m, 1H), 2.89-2.53 (m, 4H), 2.46-1.50 (m, 16H), 1.32- 1.21 (m, 3H), 0.61- 0.42 (m, 2H); 769.3 [M + H]⁺. |
| A34 | ((S)-(2-(((3S,6S, 9S,10aR)-9- (ethyl((1s,3R)-3- fluorocyclo- butyl)amino)-5- oxo-3-((S)-6- phenyl-4- azaspiro[2.4] heptane-4- carbonyl)deca- hydropyrrolo [1,2-a]azocin-6- yl)carbamoyl) benzo[b] thiophen-5-yl) fluoromethyl) phosphonic acid | 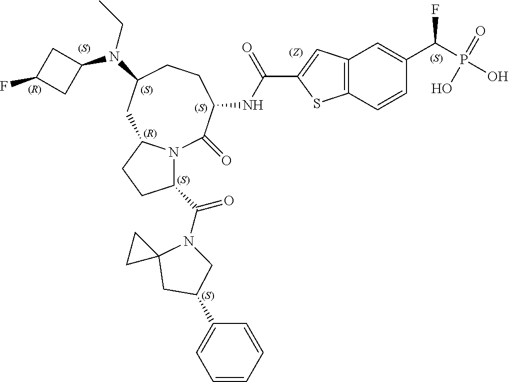 | 1H NMR (400 MHz, MeOD-d4) δ (ppm) 8.06 (s, 2H), 7.89 (d, J = 8.5 Hz, 1H), 7.61 (d, J = 8.4 Hz, 1H), 7.36-7.25 (m, 4H), 7.24-7.16 (m, 1H), 5.69 (dd, J = 45.1, 8.3 Hz, 1H), 4.84-4.73 (m, 1H), 4.69 (d, J = 11.6 Hz, 1H), 4.65-4.58 (m, 1H), 4.52-4.40 (m, 1H), 4.07-3.83 (m, 3H), 3.62-3.43 (m, 2H), 3.24-3.01 (m, 2H), 3.00-2.38 (m, 5H), 2.35-1.72 (m, 12H), 1.59 (s, 1H), 1.36 (s, 3H), 0.59- 0.45 (m, 2H); 769.4 [M + H]⁺. |
| A35 | ((R)-(2-(((3S, 6S,9S,10aR)-9- (((1s,3R)-3- (difluoromethyl) cyclobutyl) (methyl)amino)- 5-oxo-3-((S)-6- phenyl-4- azaspiro[2.4] heptane-4- carbonyl)deca- hydropyrrolo [1,2-a]azocin- 6-yl)carbamoyl) benzo[b] thiophen-5- yl)fluoromethyl) phosphonic acid | 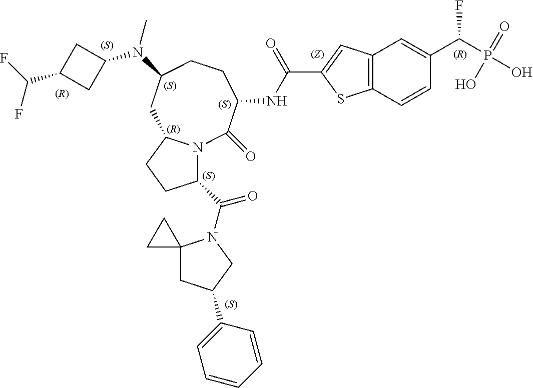 | 1H NMR (400 MHz, DMSO-d6) δ (ppm) 9.74-9.31 (m, 1H), 8.89-8.69 (m, 1H), 8.29-8.20 (m, 1H), 8.08-7.94 (m, 2H), 7.57-7.49 (m, 1H), 7.38-7.28 (m, 4H), 7.26-7.21 (m, 1H), 6.22-5.90 (m, 1H), 5.83 (dd, J = 44.4, 8.1 Hz, 1H), 4.85-4.75 (m, 1H), 4.62-4.46 (m, 2H), 4.20-4.07 (m, 1H), 3.87-3.74 (m, 2H), 3.60-3.49 (m, 2H), 2.62-2.53 (m, 3H), 2.47-1.49 (m, 19H), 0.61-0.40 (m, 2H); 787.4 [M + H]⁺. |
| A36 | ((S)-(2-(((3S, 6S,9S,10aR)-9- (((S)-3,3- difluorocyclo- pentyl)(methyl) amino)-5-oxo- 3-((S)-6-phenyl- 4-azaspiro[2.4] heptane-4- carbonyl)deca- hydropyrrolo [1,2-a]azocin-6- yl)carbamoyl) benzo[b] thiophen-5-yl) fluoromethyl) phosphonic acid | 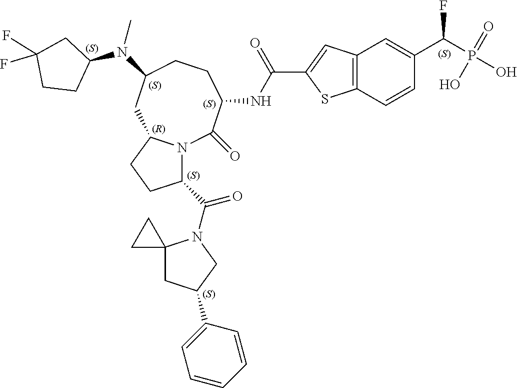 | 1H NMR (400 MHz, MeOD-d4) δ (ppm) 8.09 (d, J = 5.4 Hz, 1H), 8.03 (s, 1H), 7.98-7.89 (m, 1H), 7.58 (d, J = 8.4 Hz, 1H), 7.38-7.25 (m, 4H), 7.24-7.17 (m, 1H), 5.93-5.71 (m, 1H), 4.71-4.54 (m, 2H), 4.39-4.03 (m, 2H), 3.98-3.83 (m, 2H), 3.67-3.32 (m, 2H), 2.84-2.76 (m, 3H), 2.55-1.85 (m, 19H), 1.73- 1.58 (m, 1H), 0.62- 0.43 (m, 2H); 787.3 [M + H]⁺. |
| A37 | ((R)-(2-(((3S, 6S,9S,10aR)-9- (((1s,3R)-3- cyanocyclo- butyl)(methyl) amino)-5-oxo-3- ((S)-6-phenyl-4- azaspiro[2.4] heptane-4- carbonyl)deca- hydropyrrolo [1,2-a]azocin-6- yl)carbamoyl) benzo[b] thiophen-5-yl) fluoromethyl) phosphonic acid | 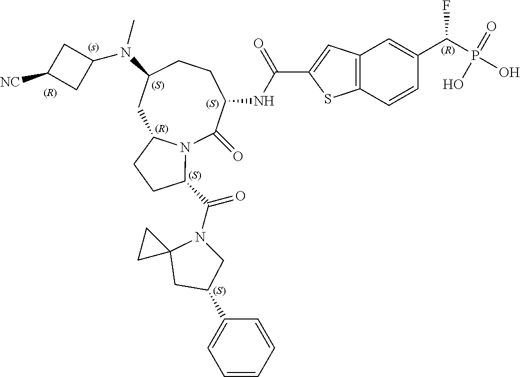 | 1H NMR (400 MHz, MeOD-d4) δ (ppm) 8.16-8.08 (m, 1H), 8.08-8.01 (m, 1H), 8.01-7.92 (m, 1H), 7.65-7.54 (m, 1H), 7.45-7.16 (m, 5H), 6.00-5.69 (m, 1H), 4.90-4.81 (m, 1H), 4.75-4.64 (m, 1H), 4.64-4.53 (m, 1H), 4.20-3.80 (m, 4H), 3.67-3.47 (m, 1H), 3.24-3.12 (m, 1H), 3.03-2.62 (m, 7H), 2.60-2.45 (m, 1H), 2.39-2.19 (m, 5H), 2.15-1.89 (m, 6H), 1.87-1.59 (m, 2H), 0.68-0.50 (m, 2H); 762.3 [M + H]⁺. |
| A38 | ((S)-fluoro(2- (((3S,6S,9S, 10aR)-9-(methyl (4,4,4-trifluoro- butyl)amino)-5- oxo-3-((S)-6- phenyl-4- azaspiro[2.4] heptane-4- carbonyl)deca- hydropyrrolo [1,2-a]azocin- 6-yl)carbamoyl) benzo[b] thiophen-5-yl) methyl) phosphonic acid | 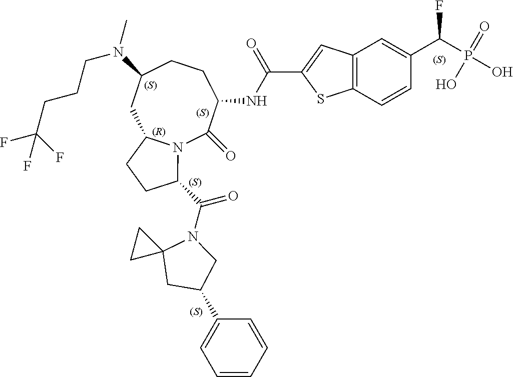 | 1H NMR (400 MHz, DMSO-d6) δ (ppm) 9.72-9.32 (m, 1H), 8.91-8.69 (m, 1H), 8.25 (s, 1H), 8.08-7.94 (m, 2H), 7.53 (d, J = 8.5 Hz, 1H), 7.38-7.28 (m, 4H), 7.25-7.20 (m, 1H), 5.81 (dd, J = 44.3, 7.9 Hz, 1H), 4.81 (s, 1H), 4.60- 4.50 (m, 2H), 4.17- 4.09 (m, 1H), 3.90- 3.69 (m, 2H), 3.63- 3.51 (m, 1H), 3.23- 3.00 (m, 2H), 2.79- 2.68 (m, 3H), 2.41- 1.75 (m, 17H), 1.58- 1.49 (m, 1H), 0.57- 0.42 (m, 2H); 793.3 [M + H]⁺. |
| A39 | ((R)-fluoro(2- (((3S,6S,9S, 10aR)-9-(((1s, 3R)-3-fluoro- cyclobutyl) (methyl)amino)- 5-oxo-3-(4- azaspiro[2.4] heptane-4- carbonyl)deca- hydropyrrolo [1,2-a]azocin-6- yl)carbamoyl) benzo[b] thiophen-5-yl) methyl) phosphonic acid | 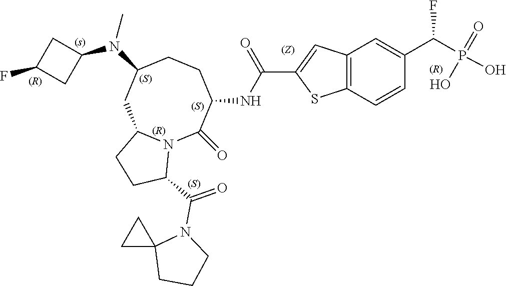 | 1H NMR (400 MHz, MeOD-d4) δ (ppm) 8.04 (d, J = 10.2 Hz, 2H), 7.94 (d, J = 8.5 Hz, 1H), 7.60 (d, J = 8.3 Hz, 1H), 5.80 (dd, J = 44.6, 8.0 Hz, 1H), 4.83-4.74 (m, 2H), 4.72-4.49 (m, 2H), 4.13-3.81 (m, 2H), 3.69-3.43 (m, 2H), 3.00-2.74 (m, 2H), 2.67 (s, 3H), 2.52- 2.41 (m, 2H), 2.30- 2.13 (m, 3H), 2.06- 1.69 (m, 13H), 0.56- 0.45 (m, 2H); 679.4 [M + H]⁺. |
| A40 | ((S)-(2-(((3S,6S, 9S,10aR)-9- (((1s,3R)-3- cyanocyclo- butyl)(methyl) amino)-5-oxo- 3-((S)-6-phenyl- 4-azaspiro[2.4] heptane-4- carbonyl)deca- hydropyrrolo [1,2-a]azocin-6- yl)carbamoyl) benzo[b] thiophen-5-yl) fluoromethyl) phosphonic acid | 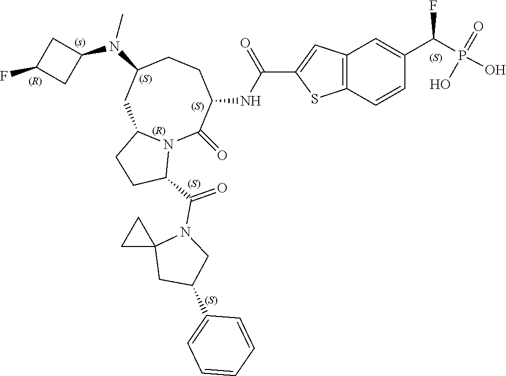 | 1H NMR (400 MHz, MeOD-d4) δ (ppm) 8.04 (s, 2H), 7.86 (d, J = 8.4 Hz, 1H), 7.63 (d, J = 8.6 Hz, 1H), 7.40-7.26 (m, 4H), 7.23-7.16 (m, 1H), 5.64 (dd, J = 45.2, 8.4 Hz, 1H), 4.78-4.71 (m, 2H), 4.64-4.52 (m, 2H), 4.46-4.35 (m, 1H), 4.12-3.88 (m, 2H), 3.65-3.52 (m, 1H), 3.02-2.86 (m, 1H), 2.70-2.57 (m, 1H), 2.55-2.15 (m, 8H), 2.11 (s, 3H), 2.02- 1.93 (m, 3H), 1.88- 1.79 (m, 2H), 1.78- 1.61 (m, 4H), 0.57- 0.46 (m, 2H); 762.4 [M + H]⁺. |
| A41 | 2,2,2-trifluoro- acetic acid compound with ((R)-(2-(((3S,6S, 9S,10aR)-9- (ethyl((3- methyloxetan-3- yl)methyl) amino)-5-oxo- 3-((S)-6-phenyl- 4-azaspiro[2.4] heptane-4- carbonyl)deca- hydropyrrolo [1,2-a]azocin-6- yl)carbamoyl) benzo[b] thiophen-5- yl)fluoromethyl) phosphonic acid | 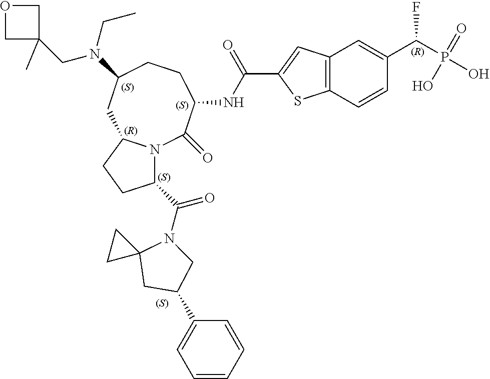 | 1H NMR (400 MHz, DMSO-d6) δ (ppm) 8.86-8.76 (m, 1H), 8.66-8.37 (m, 1H), 8.28-8.21 (m, 1H), 8.06-7.94 (m, 2H), 7.53 (d, J = 8.5 Hz, 1H), 7.39- 7.27 (m, 4H), 7.26- 7.20 (m, 1H), 5.82 (dd, J = 44.3, 8.0 Hz, 1H), 4.86-4.78 (m, 1H), 4.57-4.34 (m, 4H), 4.30-4.20 (m, 2H), 4.16-4.09 (m, 1H), 3.91-3.79 (m, 1H), 3.78-3.71 (m, 1H), 3.61-3.49 (m, 2H), 3.42-3.33 (m, 1H), 3.25-3.15 (m, 1H), 3.06-2.87 (m, 1H), 2.49-1.55 (m, 14H), 1.51 (s, 3H), 1.31 (t, J = 6.9 Hz, 3H), 0.58-0.45 |
| | | | (m, 2H); 781.4 |
| | | | [M + H]⁺. |
| A42 | ((R)-(2-(((3S, 6S,9S,10aR)-9- (ethyl((1s,3R)-3- (trifluoro- methoxy)cyclo- butyl)amino)-5- oxo-3-((S)-6- phenyl-4- azaspiro[2.4] heptane-4- carbonyl)deca- hydropyrrolo [1,2-a]azocin-6- yl)carbamoyl) benzo[b] thiophen-5-yl) fluoromethyl) phosphonic acid | 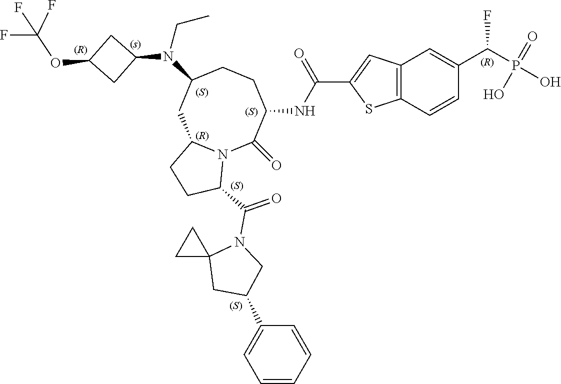 | 1H NMR (400 MHz, MeOD-d4) δ (ppm) 8.05 (d, J = 9.8 Hz, 2H), 7.93 (d, J = 8.5 Hz, 1H), 7.60 (d, J = 8.6 Hz, 1H), 7.38-7.27 (m, 4H), 7.21 (t, J = 7.3 Hz, 1H), 5.79 (dd, J = 44.8, 8.0 Hz, 1H), 4.70-4.47 (m, 4H), 4.11-3.90 (m, 3H), 3.82-3.51 (m, 3H), 3.13 (s, 1H), 2.88- 2.81 (m, 1H), 2.70- 2.42 (m, 3H), 2.39- 2.14 (m, 6H), 2.11- 1.84 (m, 8H), 1.38- 1.35 (m, 3H), 0.61- 0.48 (m, 2H); 835.4 [M + H]⁺. |
| A43 | ((R)-(2-(((3S,6S, 9S,10aR)-9- (ethyl(4,4,4- trifluorobutyl) amino)-5-oxo-3- ((S)-6-phenyl-4- azaspiro[2.4] heptane-4- carbonyl)deca- hydropyrrolo [1,2-a]azocin-6- yl)carbamoyl) benzo[b] thiophen-5-yl) fluoromethyl) phosphonic acid | 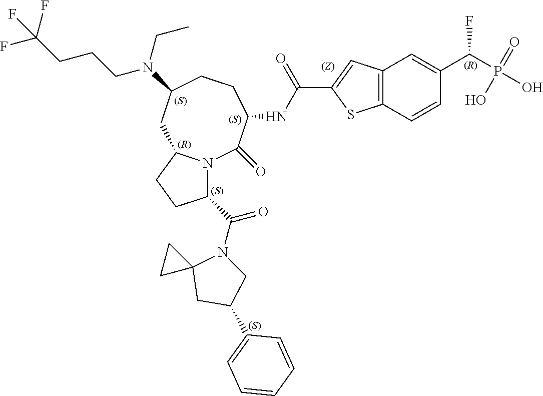 | 1H NMR (400 MHz, DMSO-d6) δ (ppm) 9.03-8.86 (m, 1H), 8.86-8.72 (m, 1H), 8.30-8.16 (m, 1H), 8.07-7.89 (m, 2H), 7.58-7.47 (m, 1H), 7.39-7.27 (m, 4H), 7.26-7.20 (m, 1H), 5.80 (dd, J = 44.4, 8.0 Hz, 1H), 4.94-4.73 (m, 1H), 4.62-4.42 (m, 2H), 4.19-4.04 (m, 1H), 3.90-3.70 (m, 2H), 3.59-3.47 (m, 2H), 3.45-3.17 (m, 3H), 2.48-2.15 (m, 4H), 2.14-1.71 (m, 13H), 1.59-1.44 (m, 1H), 1.36-1.20 (m, 3H), 0.61-0.38 (m, 2H); 807.7 [M + H]⁺. |
| A44 | ((S)-(2- (((3S,6S,9S, 10aR)-9- (ethyl(4,4,4- trifluorobutyl) amino)-5-oxo-3- ((S)-6-phenyl-4- azaspiro[2.4] heptane-4- carbonyl)deca- hydropyrrolo [1,2-a]azocin-6- yl)carbamoyl) benzo[b] thiophen-5-yl) fluoromethyl) phosphonic acid | 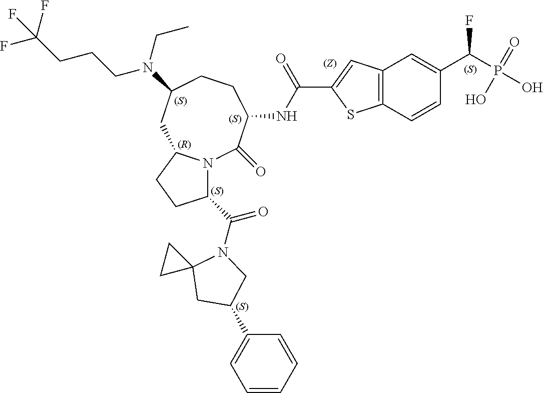 | 1H NMR (400 MHz, DMSO-d6) δ (ppm) 8.97 (s, 1H), 8.86-8.78 (m, 1H), 8.24 (d, J = 4.2 Hz, 1H), 8.03 (d, J = 8.5 Hz, 1H), 7.98 (s, 1H), 7.53 (d, J = 8.6 Hz, 1H), 7.38-7.28 (m, 4H), 7.26-7.19 (m, 1H), 5.83 (dd, J = 44.3, 8.0 Hz, 1H), 4.86-4.78 (m, 1H), 4.59-4.50 (m, 2H), 4.17-4.10 (m, 1H), 3.88-3.72 (m, 2H), 3.56-3.51 (m, 1H), 3.22-3.06 (m, 4H), 2.49-2.30 (m, 3H), 2.26-2.19 (m, 1H), 2.14-1.73 (m, 13H), 1.58-1.48 (m, 1H), 1.34-1.24 (m, 3H), 0.58-0.41 (m, 2H); 807.3 |
| | | | [M + H]⁺. |
| A45 | ((R)-(2- (((3S,6S,9S, 10aR)-9- (ethyl((1s,3R)-3- (trifluoromethyl) cyclobutyl) amino)-5-oxo-3- ((S)-6-phenyl-4- azaspiro[2.4] heptane-4- carbonyl)deca- hydropyrrolo [1,2-a]azocin-6- yl)carbamoyl) benzo[b] thiophen-5-yl) fluoromethyl) phosphonic acid | 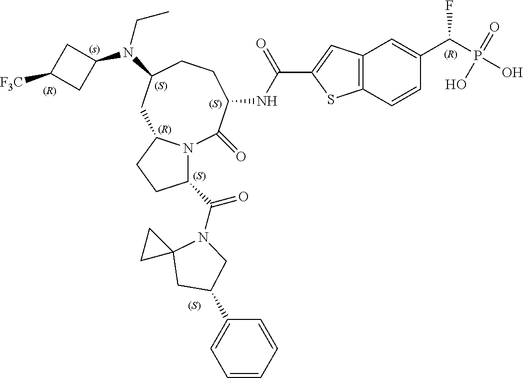 | 1H NMR (400 MHz, DMSO-d6) δ (ppm) 8.86-8.72 (m, 1H), 8.30-8.16 (m, 1H), 8.03-7.92 (m, 2H), 7.57-7.48 (m, 1H), 7.40-7.20 (m, 5H), 5.76 (dd, J = 44.6, 7.7 Hz, 1H), 4.88-4.73 (m, 1H), 4.65-4.43 (m, 2H), 4.21-4.07 (m, 1H), 4.03-3.90 (m, 1H), 3.87-3.68 (m, 3H), 3.60-3.48 (m, 2H), 3.21-3.12 (m, 1H), 2.78-2.60 (m, 1H), 2.50-1.50 (m, 17H), 1.35-1.14 (m, 3H), 0.66-0.34 (m, 2H); 819.4 [M + H]⁺. |
| A46 | ((S)-(2- (((3S,6S,9S, 10aR)-9-(ethyl ((1r,3S)-3- (trifluoromethyl) cyclobutyl) amino)-5-oxo- 3-((S)-6-phenyl- 4-azaspiro[2.4] heptane-4- carbonyl)deca- hydropyrrolo [1,2-a]azocin-6- yl)carbamoyl) benzo[b] thiophen-5-yl) fluoromethyl) phosphonic acid | 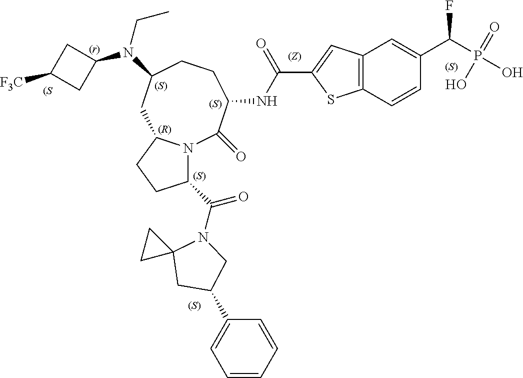 | 1H NMR (400 MHz, DMSO-d6) δ (ppm) 8.89-8.73 (m, 1H), 8.30-8.20 (m, 1H), 8.05-7.93 (m, 2H), 7.56-7.48 (m, 1H), 7.39-7.19 (m, 5H), 5.80 (dd, J = 44.3, 8.0 Hz, 1H), 4.85-4.71 (m, 1H), 4.62-4.44 (m, 2H), 4.20-4.08 (m, 1H), 4.02-3.62 (m, 4H), 3.60-3.49 (m, 1H), 3.24-3.02 (m, 2H), 2.74-2.52 (m, 2H), 2.45-1.51 (m, 16H), 1.31-1.19 (m, 3H), 0.58-0.41 (m, 2H); 819.4 [M + H]⁺. |
| A47 | ((S)-(2-(((3S,6S, 9S,10aR)-9- (ethyl((1s,3R)-3- (trifluoro- methoxy)cyclo- butyl)amino)-5- oxo-3-((S)-6- phenyl-4- azaspiro[2.4] heptane-4- carbonyl)deca- hydropyrrolo [1,2-a]azocin-6- yl)carbamoyl) benzo[b] thiophen-5-yl) fluoromethyl) phosphonic acid | 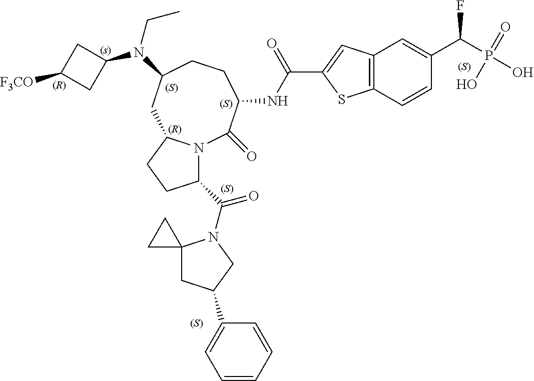 | 1H NMR (400 MHz, MeOD-d4) δ (ppm) 8.19-8.02 (m, 2H), 7.95 (d, J = 8.5 Hz, 1H), 7.62 (d, J = 8.4 Hz, 1H), 7.45-7.36 (m, 2H), 7.36-7.28 (m, 2H), 7.28-7.19 (m, 1H), 5.80 (dd, J = 44.6, 8.0 Hz, 1H), 4.86- 4.77 (m, 2H), 4.75- 4.48 (m, 3H), 4.19- 3.88 (m, 3H), 3.84- 3.53 (m, 2H), 3.29- 3.09 (m, 2H), 3.06- 2.93 (m, 1H), 2.92- 2.79 (m, 1H), 2.72- 2.58 (m, 1H), 2.57- 2.47 (m, 1H), 2.42- 2.16 (m, 5H), 2.15- 1.90 (m, 6H), 1.89- 1.71 (m, 1H), 1.69- 1.58 (m, 1H), 1.51- |
| | | | 1.34 (m, 3H), 0.71- |
| | | | 0.41 (m, 2H); 8.35.3 |
| | | | [M + H]⁺. |
| A48 | ((R)-fluoro(2- (((3S,6S,9S, 10aR)-9-(methyl (((1s,3R)-3- (trifluoromethyl) cyclobutyl) methyl)amino)- 5-oxo-3-((S)-6- phenyl-4- azaspiro[2.4] heptane-4- carbonyl)deca- hydropyrrolo [1,2-a]azocin-6- yl)carbamoyl) benzo[b] thiophen-5- yl)methyl) phosphonic acid | 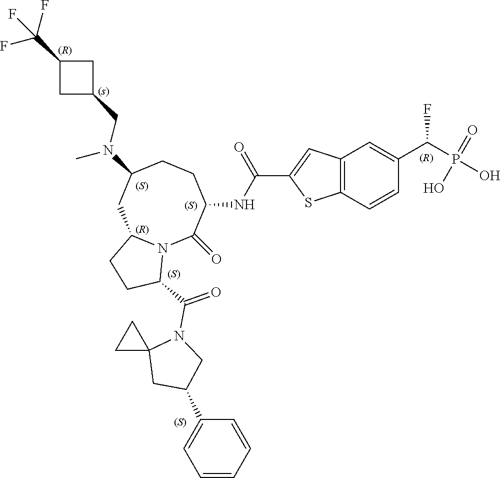 | 1H NMR (400 MHz, DMSO-d6) δ (ppm) 8.77 (dd, J = 32.3, 7.1 Hz, 1H), 8.31-8.19 (m, 1H), 8.03 (d, J = 8.4 Hz, 1H), 7.98 (s, 1H), 7.53 (d, J = 8.6 Hz, 1H), 7.42-7.17 (m, 5H), 5.83 (dd, J = 44.4, 8.1 Hz, 1H), 4.86-4.73 (m, 1H), 4.60-4.46 (m, 2H), 4.13 (t, J = 8.3 Hz, 1H), 3.79-3.69 (m, 2H), 3.58-3.52 (m, 1H), 3.27-2.95 (m, 4H), 2.84-2.57 (m, 4H), 2.42-1.64 (m, 16H), 1.63-1.51 (m, 1H), 0.54 (s, 2H); 819.5 [M + H]⁺. |
| A49 | ((R)-fluoro(2- (((3S,6S,9S, 10aR)-9- (methyl(((1s, 3R)-3- (trifluoromethyl) cyclobutyl) methyl)amino)- 5-oxo-3-((S)- 6-phenyl-4- azaspiro[2.4] heptane-4- carbonyl)deca- hydropyrrolo [1,2-a]azocin-6- yl)carbamoyl) benzo[b] thiophen-5-yl) methyl) phosphonic acid | 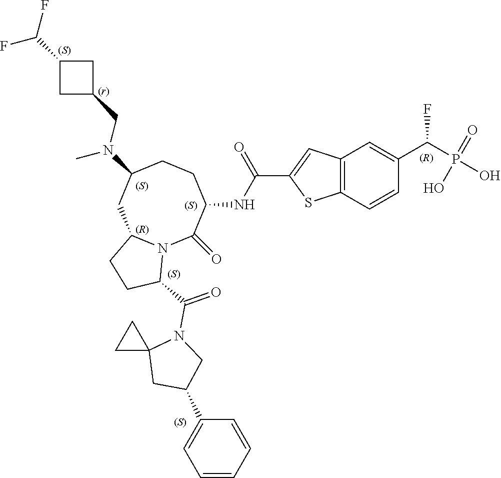 | 1H NMR (400 MHz, MeOD-d4) δ (ppm) 8.13-8.09 (m, 1H), 8.08-7.96 (m, 2H), 7.96-7.92 (m, 1H), 7.63-7.26 (m, 4H), 7.37-7.56 (m, 1H), 7.23-7.17 (m, 1H), 6.15-5.95 (m, 1H), 5.93-5.79 (m, 1H), 4.89-4.79 (m, 1H), 4.74-4.51 (m, 2H), 4.12-3.87 (m, 3H), 3.68-3.36 (m, 2H), 3.27-3.18 (m, 1H), 2.92-2.69 (m, 5H), 2.57-2.44 (m, 1H), 2.38-1.91 (m, 15H), 1.72- 1.57 (m, 1H), 0.63- 0.47 (m, 2H); 801.5 [M + H]⁺. |
| A50 | ((R)-fluoro(2- (((3S,6S,9S, 10aR)-9-(methyl (2-(1- (trifluoromethyl) cyclopropyl) ethyl)amino)-5- oxo-3-((S)-6- phenyl-4- azaspiro[2.4] heptane-4- carbonyl)deca- hydropyrrolo [1,2-a]azocin-6- yl)carbamoyl) benzo[b] thiophen-5- yl)methyl) phosphonic acid | 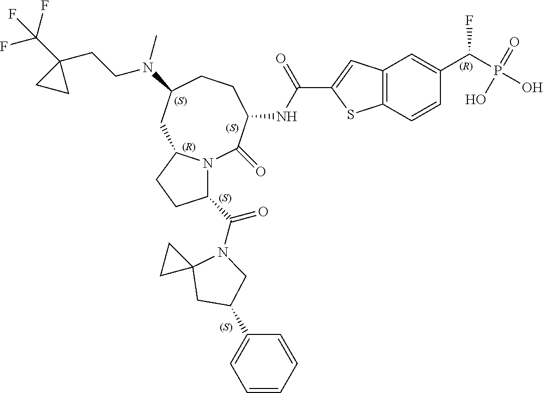 | 1H NMR (400 MHz, MeOD-d4) δ (ppm) 8.11-8.07 (m, 1H), 8.05 (s, 1H), 7.99-7.92 (m, 1H), 7.63 (d, J = 8.1 Hz, 1H), 7.40-7.27 (m, 4H), 7.26-7.20 (m, 1H), 5.81 (dd, J = 44.6, 7.7 Hz, 1H), 4.82-4.70 (m, 1H), 4.70-4.59 (m, 1H), 4.58-4.44 (m, 1H), 4.13-3.86 (m, 3H), 3.67-3.35 (m, 2H), 3.30-3.17 (m, 1H), 2.90-2.69 (m, 3H), 2.57-2.44 (m, 1H), 2.39-2.27 (m, 1H), 2.26-1.76 (m, 13H), 1.71-1.62 (m, 1H), 1.14-1.02 (m, 2H), 0.93-0.82 (m, 2H), 0.62-0.47 (m, 2H); 819.3 [M + H]⁺. |
| A51 | ((R)-fluoro(2- (((3S,6S,9S, 10aR)-9- (methyl((1s,3R)- 3-(trifluoro- methoxy)cyclo- butyl)amino)-5- oxo-3-(4- azaspiro[2.4] heptane-4- carbonyl) decahydro- pyrrolo[1,2-a] azocin-6-yl) carbamoyl) benzo[b] thiophen-5-yl) methyl) | 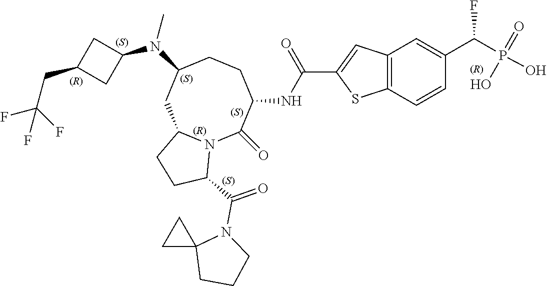 | 1H NMR (400 MHz, MeOD-d4) δ (ppm) 8.13-7.97 (m, 2H), 7.96-7.78 (m, 1H), 7.68-7.50 (m, 1H), 5.66 (dd, J = 45.2, 8.4 Hz, 1H), 4.71-4.59 (m, 2H), 4.58-4.48 (m, 1H), 4.48-4.33 (m, 1H), 3.98-3.84 (m, 1H), 3.83-3.67 (m, 1H), 3.66-3.56 (m, 1H), 3.52-3.36 (m, 1H), 3.01-2.88 (m, 1H), 2.84-2.70 (m, 1H), 2.68-2.37 (m, 6H), 2.29-2.08 (m, 1H), |
| | phosphonic | | 2.05-1.66 (m, 14H), |
| | acid | | 0.64-0.28 (m, 2H); |
| | | | 745.5 [M + H]⁺. |
| A52 | ((R)-fluoro(2- (((3S,6S,9S, 10aR)-9-(methyl (3-(trifluoro- methoxy)propyl) amino)-5-oxo-3- ((S)-6-phenyl- 4-azaspiro[2.4] heptane-4- carbonyl)deca- hydropyrrolo [1,2-a]azocin-6- yl)carbamoyl) benzo[b] thiophen-5- yl)methyl) phosphonic acid | 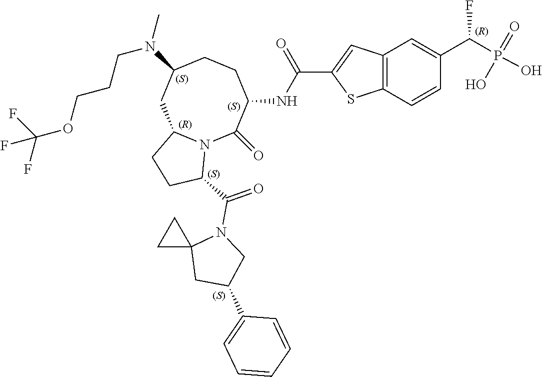 | 1H NMR (400 MHz, DMSO-d6) δ (ppm) 10.87-9.92 (m, 1H), 8.73 (s, 1H), 8.23 (s, 1H), 8.09-7.72 (m, 2H), 7.63-7.41 (m, 1H), 7.39-7.10 (m, 5H), 5.63 (dd, J = 45.1, 7.8 Hz, 1H), 4.82- 4.60 (m, 1H), 4.56- 4.35 (m, 2H), 4.20- 4.01 (m, 3H), 3.79- 3.60 (m, 2H), 3.55- 3.47 (m, 1H), 3.22- 2.95 (m, 2H), 2.84- 2.53 (m, 3H), 2.45- 2.37 (m, 1H), 2.24- 1.63 (m, 14H), 1.58- 1.49 (m, 1H), 0.57- 0.29 (m, 2H); 809.5 [M + H]⁺. |
| A53 | ((R)-fluoro(2- (((3S,6S,9S, 10aR)-9-(methyl ((S)-4,4,4- trifluoro-3- methoxybutyl) amino)-5-oxo-3- ((S)-6-phenyl-4- azaspiro[2.4] heptane-4- carbonyl)deca- hydropyrrolo [1,2-a]azocin-6- yl)carbamoyl) benzo[b] thiophen-5-yl) methyl) phosphonic acid | 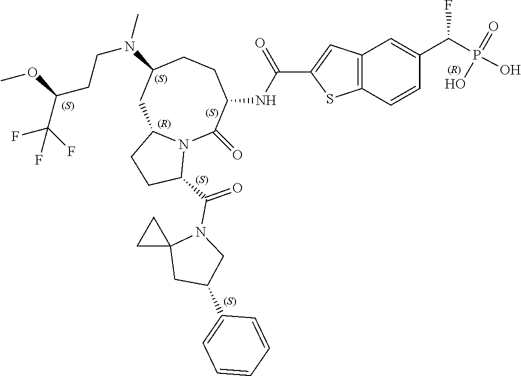 | 1H NMR (400 MHz, MeOD-d4) δ (ppm) 8.03-7.89 (m, 2H), 7.86-7.75 (m, 1H), 7.51 (d, J = 8.4 Hz, 1H), 7.32- 7.01 (m, 5H), 5.69 (dd, J = 44.7, 8.0 Hz, 1H), 4.71-4.62 (m, 1H), 4.59-4.48 (m, 1H), 4.46-4.34 (m, 1H), 4.01-3.67 (m, 4H), 3.56-3.25 (m, 5H), 3.10-2.83 (m, 1H), 2.80-2.60 (m, 3H), 2.46-2.32 (m, 1H), 2.26-1.72 (m, 14H), 1.62-1.46 (m, 1H), 0.54-0.23 (m, 2H); 823.4 [M + H]⁺. |
| A54 | ((R)-fluoro(2- (((3S,6S,9S, 10aR)-9- morpholino-5- oxo-3-((S)-6- phenyl-4- azaspiro[2.4] heptane-4- carbonyl)deca- hydropyrrolo [1,2-a]azocin-6- yl)carbamoyl) benzo[b] thiophen-5-yl) methyl) phosphonic acid | 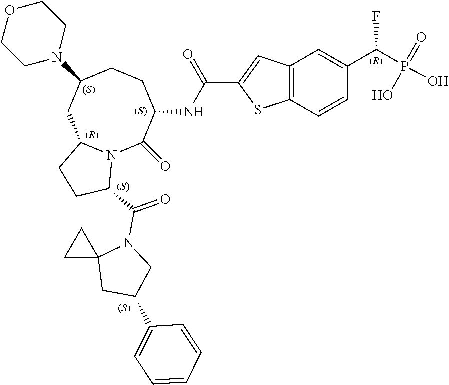 | 1H NMR (400 MHz, DMSO-d6) δ (ppm) 8.82-8.70 (m, 1H), 8.25-8.20 (m, 1H), 8.06-7.91 (m, 2H), 7.56-7.49 (m, 1H), 7.38-7.26 (m, 4H), 7.26-7.19 (m, 1H), 5.77 (dd, J = 44.5, 8.1 Hz, 1H), 4.82-4.71 (m, 1H), 4.57-4.41 (m, 2H), 4.16-4.09 (m, 1H), 4.04-3.87 (m, 2H), 3.74-3.48 (m, 5H), 3.23-3.06 (m, 4H), 2.35-1.95 (m, 8H), 1.93-1.48 (m, 6H), 0.58-0.40 (m, 2H); 739.4 [M + H]⁺. |
| A55 | ((R)-(2- (((3S,6S,9S, 10aR)-9-(((R)- 3,3-difluoro- cyclopentyl) (methyl) amino)-5-oxo-3- ((S)-6-phenyl-4- azaspiro[2.4] heptane-4- carbonyl)deca- hydropyrrolo [1,2-a]azocin-6- yl)carbamoyl) benzo[b] thiophen-5-yl) fluoromethyl) phosphonic acid | 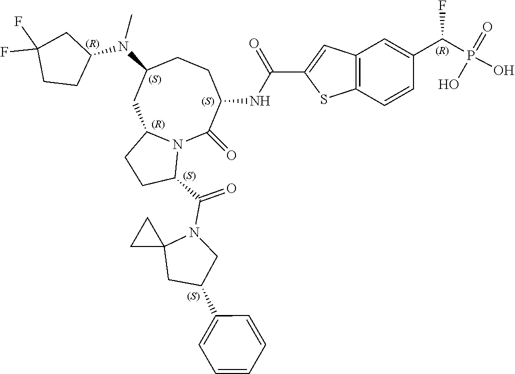 | 1H NMR (400 MHz, DMSO-d6) δ (ppm) 8.86-8.64 (m, 1H), 8.23 (s, 1H), 8.04-7.92 (m, 2H), 7.53 (d, J = 8.4 Hz, 1H), 7.37-7.28 (m, 4H), 7.25-7.18 (m, 1H), 5.72 (dd, J = 44.7, 7.5 Hz, 1H), 4.87-4.71 (m, 1H), 4.60-4.46 (m, 2H), 4.18-4.01 (m, 2H), 3.84-3.71 (m, 2H), 3.58-3.50 (m, 1H), 2.65 (s, 3H), 2.48- 2.20 (m, 5H), 2.15- 1.62 (m, 14H), 1.57- 1.48 (m, 1H), 0.59- 0.44 (m, 2H); 787.3 [M + H]⁺. |
| A56 | ((R)-fluoro(2- (((3S,6S,9S, 10aR)-9-(methyl ((3-methyl- oxetan-3-yl) methyl)amino)- 5-oxo-3-((S)-6- phenyl-4- azaspiro[2.4] heptane-4- carbonyl)deca- hydropyrrolo [1,2-a]azocin-6- yl)carbamoyl) benzo[b] thiophen-5-yl) methyl) phosphonic acid | 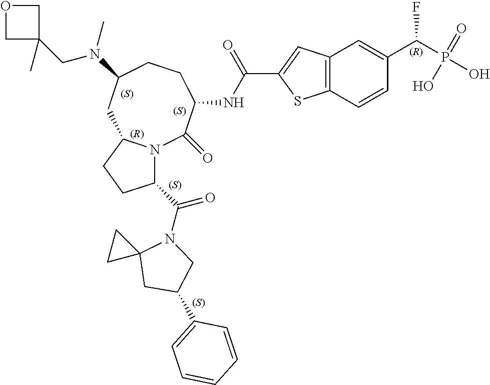 | 1H NMR (400 MHz, MeOD-d4) δ (ppm) 8.09-7.99 (m, 2H), 7.88 (d, J = 8.5 Hz, 1H), 7.65 (d, J = 8.5 Hz, 1H), 7.36-7.26 (m, 4H), 7.23-7.18 (m, 1H), 5.64 (dd, J = 45.3, 8.3 Hz, 1H), 4.75- 4.67 (m, 1H), 4.63- 4.54 (m, 3H), 4.44- 4.30 (m, 3H), 4.06- 3.87 (m, 2H), 3.61- 3.46 (m, 2H), 3.26- 3.07 (m, 2H), 2.55 (s, 3H), 2.48-2.39 (m, 1H), 2.30 (t, J = 11.4 Hz, 1H), 2.22- 1.92 (m, 7H), 1.92- 1.75 (m, 4H), 1.66- 1.59 (m, 1H), 1.52 (s, 3H), 0.57-0.44 (m, 2H); 767.4 [M + H]⁺. |
| A57 | ((S)-fluoro(2- (((3S,6S,9S, 10aR)-9-(methyl ((1R,3S)-3- (trifluoro- methoxy)cyclo- pentyl)amino)- 5-oxo-3-((S)-6- phenyl-4- azaspiro[2.4] heptane-4- carbonyl)deca- hydropyrrolo [1,2-a]azocin-6- yl)carbamoyl) benzo[b] thiophen-5-yl) methyl) phosphonic acid | 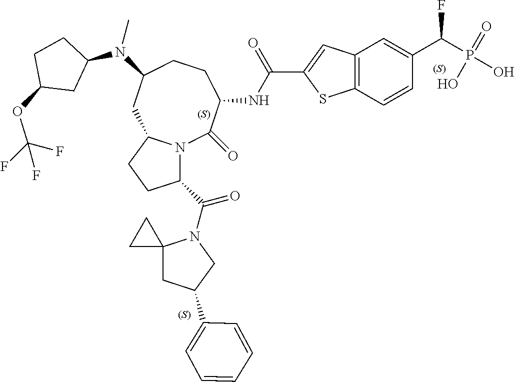 | 1H NMR (400 MHz, MeOD-d4) δ (ppm) 8.11-8.04 (m, 2H), 7.95 (d, J = 8.5 Hz, 1H), 7.62 (d, J = 8.3 Hz, 1H), 7.41-7.19 (m, 5H), 5.79 (dd, J = 44.7, 8.2 Hz, 1H), 4.80- 4.42 (m, 3H), 4.26- 3.51 (m, 5H), 2.83- 1.61 (m, 24H), 0.61- 0.48 (m, 2H); 835.2 [M + H]⁺. |
| A58 | (difluoro(2- (((3S,6S,9S, 10aR)-9-(methyl ((1s,3R)-3- (trifluoro- methoxy)cyclo- butyl)amino)-5- oxo-3-((S)-6- phenyl-4- azaspiro[2.4] heptane-4- carbonyl)deca- hydropyrrolo [1,2-a]azocin-6- yl)carbamoyl) benzo[b] thiophen-5-yl) methyl) phosphonic acid | 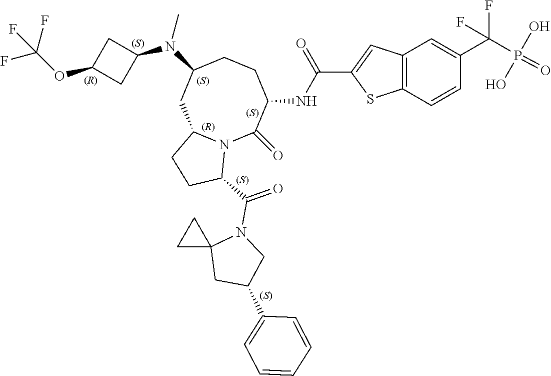 | 1H NMR (400 MHz, DMSO-d6) δ (ppm) 8.86 (s, 1H), 8.45- 8.26 (m, 1H), 8.21- 8.07 (m, 1H), 8.01 (d, J = 8.5 Hz, 1H), 7.71-7.56 (m, 1H), 7.37-7.13 (m, 5H), 4.71-4.26 (m, 4H), 4.10-4.00 (m, 1H), 3.73-3.67 (m, 1H), 3.52-3.43 (m, 2H), 3.42-3.27 (m, 2H), 3.10-2.92 (m, 1H), 2.83-2.56 (m, 3H), 2.43-2.29 (m, 3H), 2.22-2.14 (m, 1H), 2.07-1.31 (m, 12H), 0.60-0.32 (m, 2H), 839.5 [M + H]⁺. |
| A59 | ((R)-fluoro(2- (((3S,6S,9S, 10aR)-9- (methyl((1s,3R)- 3-(trifluoro- methoxy)cyclo- butyl)amino)-5- oxo-3-((R)-6- (pyridin-3-yl)- 4-azaspiro[2.4] heptane-4- carbonyl)deca- hydropyrrolo [1,2-a]azocin-6- yl)carbamoyl) benzo[b] thiophen-5-yl) methyl) phosphonic acid | 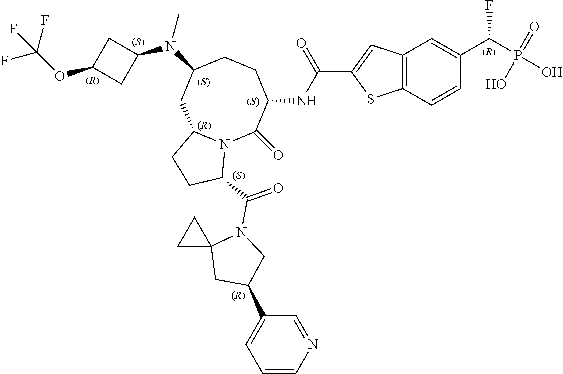 | 1H NMR (400 MHz, DMSO-d6) δ (ppm) 10.20-9.46 (m, 1H), 8.91-8.48 (m, 3H), 8.24 (s, 1H), 8.05-7.92 (m, 3H), 7.54 (d, J = 8.6 Hz, 2H), 5.83 (dd, J = 44.3, 8.0 Hz, 1H), 4.85-4.77 (m, 1H), 4.70-4.65 (m, 1H), 4.61-4.53 (m, 2H), 4.20-4.16 (m, 2H), 3.84-3.82 (m, 2H), 2.86-2.56 (m, 6H), 2.49-2.40 (m, 2H), 2.28-1.56 (m, 14H), 0.62-0.42 (m, 2H); 822.3 [M + H]⁺. |
| A60 | ((R)-fluoro(2- (((3S,6S,9S, 10aR)-9-(methyl ((1s,3R)-3- (trifluoro- methoxy)cyclo- butyl)amino)-5- oxo-3-((S)-6- (pyridin-3-yl)- 4-azaspiro[2.4] heptane-4- carbonyl)deca- hydropyrrolo [1,2-a]azocin-6- yl)carbamoyl) benzo[b] thiophen-5-yl) methyl) phosphonic acid | 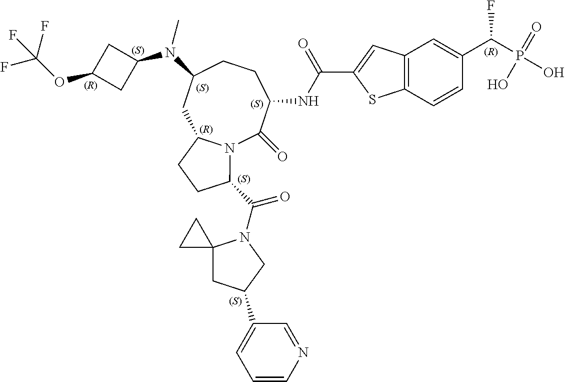 | 1H NMR (400 MHz, DMSO-d6) δ (ppm) 9.86-9.43 (m, 1H), 8.92-8.71 (m, 1H), 8.65 (s, 1H), 8.57-8.48 (m, 1H), 8.28-8.20 (m, 1H), 8.07-7.90 (m, 1H), 7.57-7.44 (m, 2H), 5.84 (dd, J = 44.3, 8.1 Hz, 1H), 4.86-4.75 (m, 1H), 4.70-4.36 (m, 5H), 3.68-3.67 (m, 2H), 2.83-2.74 (m, 1H), 2.70-2.54 (m, 5H), 2.46-2.27 (m, 3H), 2.16-1.51 (m, 13H), 0.59-0.44 (m, 2H); 822.4 [M + H]⁺. |
| A61 | ((R)-fluoro(2- (((3S,6S,9S, 10aR)-3-((6S, 7S)-7-methoxy- 6-phenyl-4- azaspiro[2.4] heptane-4- carbonyl)-9- (methyl((1s,3R)- 3-(trifluoro- methoxy)cyclo- butyl)amino)-5- oxodecahydro- pyrrolo[1,2-a] azocin-6-yl) carbamoyl) benzo[b] thiophen-5- yl)methyl) phosphonic acid | 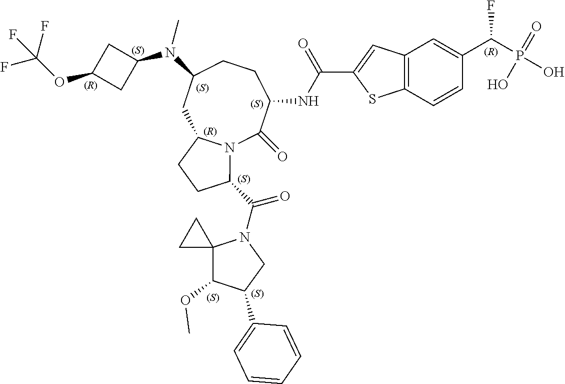 | 1H NMR (400 MHz, MeOD-d4) δ (ppm) 8.13-8.02 (m, 2H), 7.96-7.88 (m, 1H), 7.71-7.60 (m, 1H), 7.45-7.25 (m, 5H), 5.72 (dd, J = 44.8, 7.7 Hz, 1H), 4.81-4.36 (m, 5H), 4.05-3.95 (m, 1H), 3.78-3.58 (m, 2H), 3.51-3.39 (m, 1H), 3.08 (s, 3H), 2.89- 2.79 (m, 1H), 2.73- 2.63 (m, 3H), 2.57- 2.44 (m, 2H), 2.32- 2.16 (m, 3H), 2.08- 1.98 (m, 3H), 1.94- 1.89 (m, 1H), 1.61- 1.52 (m, 1H), 1.41- 1.27 (m, 5H), 0.97- 0.85 (m, 2H), 0.75-0.63 (m, 1H); 851.4 [M + H]⁺. |
| | | Absolute stereochemistry of cis-3-OMe-4-Ph pyrrolidine arbitrarily assigned | |
| A62 | ((R)-fluoro(2- (((3S,6S,9S, 10aR)-3-((6R, 7R)-7-methoxy- 6-phenyl-4- azaspiro[2.4] heptane-4- carbonyl)-9- (methyl((1s,3R)- 3-(trifluoro- methoxy)cyclo- butyl)amino)-5- oxodecahydro- pyrrolo[1,2-a] azocin-6-yl) carbamoyl) benzo[b] thiophen-5- yl)methyl) phosphonic acid | 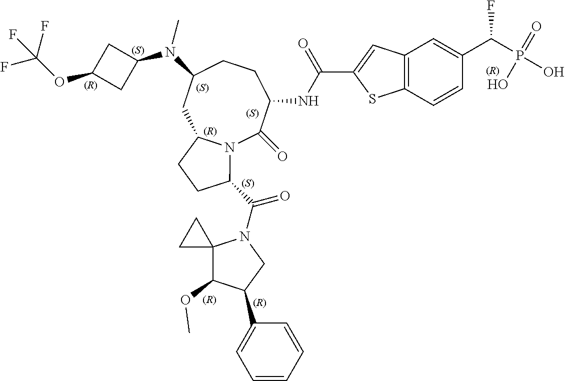 | 1H NMR (400 MHz, DMSO-d6) δ (ppm) 9.68-8.64 (m, 1H), 8.25 (s, 1H), 8.05-7.89 (m, 2H), 7.60-7.46 (m, 1H), 7.42-7.17 (m, 5H), 5.87-5.54 (m, 1H), 4.85-4.44 (m, 4H), 4.17-3.98 (m, 3H), 3.68-3.65 (m, 2H), 3.02 (s, 3H), 2.75-2.52 (m, 5H), 2.47-2.31 (m, 2H), 2.22-1.22 (m, 13H), 0.88-0.58 (m, 2H); 851.1 [M + H]⁺. |
| | | Absolute stereochemistry of cis-3-OMe-4-Ph pyrrolidine arbitrarily assigned | |
| A63 | ((R)-fluoro(2- (((3S,6S,9S, 10aR)-9-(methyl ((S)-3,3,3- trifluoro-2- methoxypropyl) amino)-5-oxo-3- ((S)-6-phenyl-4- azaspiro[2.4] heptane-4- carbonyl)deca- hydropyrrolo [1,2-a]azocin-6- yl)carbamoyl) benzo[b] thiophen-5- yl)methyl) phosphonic acid | 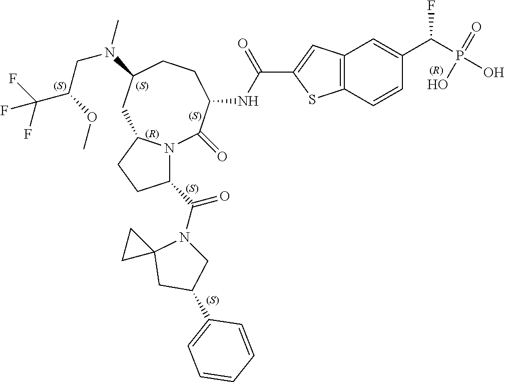 | 1H NMR (400 MHz, MeOD-d4) δ (ppm) 8.10-8.06 (m, 1H), 8.05 (s, 1H), 7.95 (d, J = 8.5 Hz, 1H), 7.65 (d, J = 8.5 Hz, 1H), 7.37 (d, J = 7.3 Hz, 2H), 7.32 (t, J = 7.6 Hz, 2H), 7.23 (t, J = 7.2 Hz, 1H), 5.78 (dd, J = 44.8, 8.1 Hz, 1H), 5.25-4.91 (m, 1H), 4.78 (d, J = 10.8 Hz, 1H), 4.70-4.63 (m, 1H), 4.59-4.49 (m, 1H), 4.12-3.85 (m, 3H), 3.74 (s, 3H), 3.65-3.52 (m, 1H), 3.38 (s, 1H), 2.92 (s, 3H), 2.58-2.44 (m, 1H), 2.34 (t, J = 11.5 Hz, 1H), 2.27-1.80 (m, 12H), 1.74- 1.61 (m, 1H), 0.64-0.45 |
| | | | (m, 2H); 809.2 |
| | | | [M + H]⁺. |
| A64 | ((R)-fluoro(2- (((3S,6S,9S, 10aR)-9-(methyl (2-(trifluoro- methoxy)ethyl) amino)-5-oxo-3- ((S)-6-phenyl- 4-azaspiro[2.4] heptane-4- carbonyl)deca- hydropyrrolo [1,2-a]azocin-6- yl)carbamoyl) benzo[b] thiophen-5-yl) methyl) phosphonic acid | 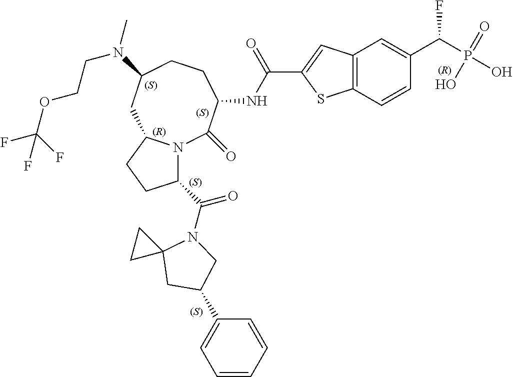 | 1H NMR (400 MHz, DMSO-d6) δ (ppm) 8.84-8.60 (m, 1H), 8.34-8.11 (m, 1H), 8.05-7.86 (m, 2H), 7.59-7.45 (m, 1H), 7.37-7.19 (m, 5H), 5.70 (dd, J = 44.6, 7.8 Hz, 1H), 4.83-4.67 (m, 1H), 4.58-4.34 (m, 4H), 4.15-4.08 (m, 1H), 3.76-3.70 (m, 1H), 3.57-3.49 (m, 2H), 3.18-3.03 (m, 1H). 2.81-2.55 (m, 3H), 2.45-2.35 (m, 1H), 2.26-2.18 (m, 1H), 2.12-1.68 (m, 12H), 1.60-1.54 (m, 1H), 0.55- 0.41 (m, 2H); 795.3 [M + H]⁺. |
| A65 | ((S)-fluoro(2- (((3S,6S,9S, 10aR)-9-(methyl ((1s,3R)-3- (trifluoromethyl) cyclobutyl) amino)-5-oxo-3- ((S)-6-phenyl-4- azaspiro[2.4] heptane-4- carbonyl)deca- hydropyrrolo [1,2-a]azocin-6- yl)carbamoyl) benzo[b] thiophen-5-yl) methyl) phosphonic acid | 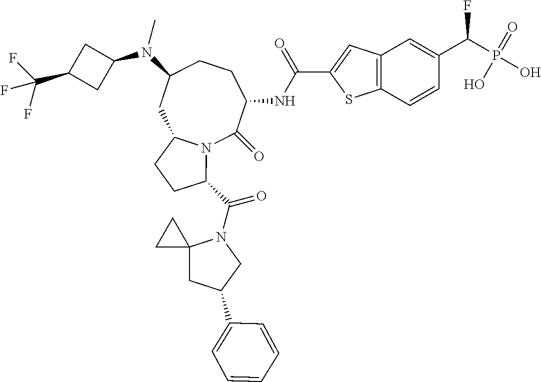 [SMILES: CN([C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1)[C@H]1C[C@@H](C(F)(F)F)C1] | 1H NMR (400 MHz, CD₃OD-d4) δ (ppm) 8.08 (d, J = 8.7 Hz, 2H), 7.89 (d, J = 8.4 Hz, 1H), 7.61 (d, J = 8.4 Hz, 1H), 7.36-7.15 (m, 5H), 5.79-5.50 (m, 1H), 4.71-4.50 (m, 2H), 4.48-4.31 (m, 1H), 4.10-3.65 (m, 4H), 3.57-3.46 (m, 1H), 2.99-2.50 (m, 6H), 2.45-1.55 (m, 16H), 0.60-0.35 (m, 2H); 805 [M + H]⁺. |

TABLE 1B

| No. | Name | Structure | NMR and LCMS |
|---|---|---|---|
| B1 | (((S)-fluoro(2- (((3S,6S,9S,10a R)-9-(((1s,3R)-3- fluorocyclobutyl) (methyl)amino)- 5-oxo-3-((S)-6- phenyl-4-azaspiro [2.4]heptane-4- carbonyl) decahydropyrrolo [1,2-a]azocin-6- yl)carbamoyl) benzo[b]thiophen-5- yl)methyl)(hydroxy) phosphoryl)-L-alanine | 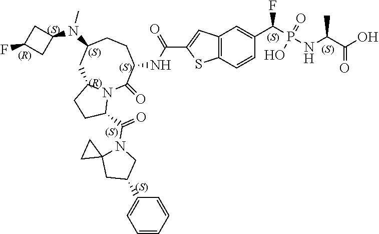 | 1H NMR (400 MHz, MeOD-d4) δ (ppm) 8.05 (s, 2H), 7.86 (d, J = 8.4 Hz, 1H), 7.60 (d, J = 8.5 Hz, 1H), 7.36 − 7.26 (m, 4H), 7.20 (t, J = 7.0 Hz, 1H), 7.17 − 6.74 (m, 1H), 5.66 (dd, J = 45.3, 7.0 Hz, 1H), 4.79 − 4.55 (m, 3H), 4.48 − 4.39 (m, 1H), 4.00 (t, J = 8.7 Hz, 1H), 3.94 − 3.75 (m, 3H), 3.59 − |
| | | | 3.48 (m, 1H), 3.39 − |
| | | | 3.32 (m, 1H), 3.01 − |
| | | | 2.90 (m, 1H), 2.80 − |
| | | | 2.70 (m, 1H), 2.69 − |
| | | | 2.57 (m, 1H), 2.55 |
| | | | (s, 3H), 2.50 − 2.37 |
| | | | (m, 2H), 2.31 (t, J = |
| | | | 11.5 Hz, 1H), 2.22 − |
| | | | 2.11 (m, 2H), 2.06 |
| | | | (d, J = 12.0 Hz, 1H), |
| | | | 2.01 − 1.69 (m, 8H), |
| | | | 1.64 − 1.56 (m, 1H), |
| | | | 1.32 (d, J = 7.0 Hz, |
| | | | 3H), 0.57 − 0.43 (m, |
| | | | 2H); 826.5 [M + H]⁺. |
| B2 | (((R)-fluoro(2-(((3S,6S,9S,10a R)-9-(methyl((1s,3R)- 3-(trifluoromethoxy) cyclobutyl)amino)-5-oxo-3- ((S)-6-phenyl-4-azaspiro [2.4]heptane-4-carbonyl) decahydropyrrolo[1,2- a]azocin-6-yl)carbamoyl) benzo[b]thiophen-5- yl)methyl)(hydroxy) phosphoryl)- L-alanine | 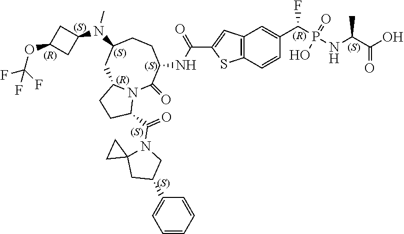 | 1H NMR (400 MHz, MeOD-d4) δ (ppm) 8.03 (d, J = 16.1 Hz, 2H), 7.82 (d, J = 8.5 Hz, 1H), 7.63 (d, J = 8.5 Hz, 1H), 7.37 − 7.26 (m, 4H), 7.22 − 7.15 (m, 1H), 5.61 (dd, J = 45.3, 7.2 Hz, 1H), 4.75 (d, J = 10.3 Hz, 1H), 4.59 (t, J = 8.7 Hz, 1H), 4.50 − 4.43 (m, 1H), 4.41 − 4.34 (m, 1H), 4.04 (t, J = |
| | | | 8.7 Hz, 1H), 3.91 (t, |
| | | | J = 9.8 Hz, 1H), |
| | | | 3.74 − 3.64 (m, 1H), |
| | | | 3.60 − 3.50 (m, 1H), |
| | | | 3.29 − 3.22 (m, 1H), |
| | | | 2.87 − 2.76 (m, 1H), |
| | | | 2.74 − 2.64 (m, 1H), |
| | | | 2.60 − 2.50 (m, 1H), |
| | | | 2.44 − 2.25 (m, 4H), |
| | | | 2.22 − 2.05 (m, 6H), |
| | | | 2.02 − 1.91 (m, 3H), |
| | | | 1.88 − 1.58 (m, 6H), |
| | | | 1.30 (d, J = 6.9 Hz, |
| | | | 3H), 0.54 − 0.43 (m, |
| | | | 2H); 892.4 [M + H]⁺. |
| B3 | (((S)-fluoro(2- (((3S,6S,9S,10aR)-9- (methyl((1s,3R)-3- (trifluoromethoxy) cyclobutyl)amino)-5-oxo-3- ((S)-6-phenyl-4-azaspiro [2.4]heptane-4-carbonyl) decahydropyrrolo[1,2- a]azocin-6-yl)carbamoyl) benzo[b]thiophen-5- yl)methyl)(hydroxy) phosphoryl)- L-alanine | 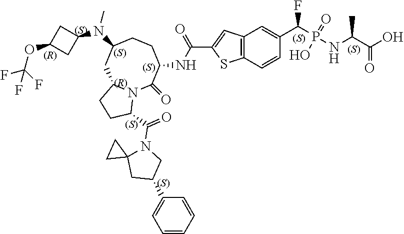 | 1H NMR (400 MHz, DMSO-d6) δ (ppm) 8.76 (s, 1H), 8.21 (s, 1H), 7.92 (d, J = 8.0 Hz, 2H), 7.53 (d, J = 8.5 Hz, 1H), 7.38 − 7.28 (m, 4H), 7.22 (t, J = 7.0 Hz, 2H), 5.63 (dd, J = 45.6, 7.3 Hz, 1H), 4.71 (s, 1H), 4.61 − 4.45 (m, 2H), 4.35 (s, 1H), 4.12 (t, J = 8.4 Hz, 1H), 3.74 (t, J = 9.9 Hz, 1H), |
| | | | 3.58 − 3.42 (m, 3H), |
| | | | 2.78 − 2.65 (m, 1H), |
| | | | 2.42 − 1.49 (m, |
| | | | 20H), 1.10 (d, J = |
| | | | 6.8 Hz, 3H), 0.56 − |
| | | | 0.39 (m, 2H); 892.5 |
| | | | [M + H]⁺. |
| B4 | (((R)-fluoro(2-(((3S,6S,9S, 10aR)-9-(methyl((1s,3R)- 3-(trifluoromethyl) cyclobutyl)amino)-5- oxo-3-((S)-6-phenyl-4- azaspiro[2.4]heptane-4- carbonyl)decahydropyrrolo [1,2-a]azocin-6- yl)carbamoyl)benzo [b]thiophen-5- yl)methyl)(hydroxy) phosphoryl)- L-alanine | 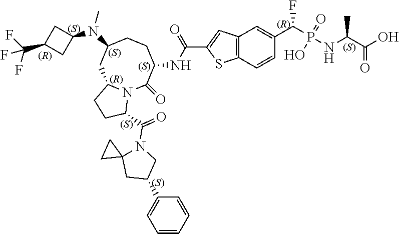 | 1H NMR (400 MHz, DMSO-d6) δ (ppm) 8.79 (s, 1H), 8.22 (s, 1H), 7.92 (d, J = 8.5 Hz, 2H), 7.56 (d, J = 8.5 Hz, 1H), 7.36 − 7.28 (m, 4H), 7.26 − 6.95 (m, 2H), 5.64 (dd, J = 45.7, 7.4 Hz, 1H), 4.70 (s, 1H), 4.58 − 4.33 (m, 2H), 4.14 − 4.05 (m, 1H), 3.81 3.38 (m, 5H), 2.96 − |
| | | | 2.78 (m, 1H), 2.46- |
| | | | 1.48 (m, 21H), 1.09 |
| | | | (d, J = 6.8 Hz, 3H), |
| | | | 0.56 − 0.39 (m, 2H); |
| | | | 876.4 [M + H]⁺. |
| B5 | ((S)-((R)-fluoro(2-(((3S,6S, 9S,10aR)-9-(methyl(4,4,4- trifluorobutyl)amino)-5- oxo-3-((S)-6-phenyl-4- azaspiro[2.4]heptane-4- carbonyl)decahydropyrrolo [1,2-a]azocin-6- yl)carbamoyl) benzo[b]thiophen-5- yl)methyl)(hydroxy) phosphoryl)- L-alanine | 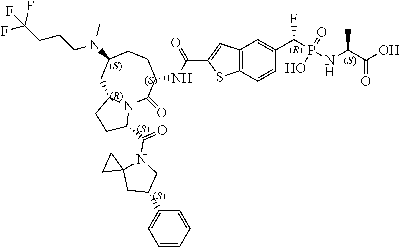 | 1H NMR (400 MHz, DMSO-d6) δ (ppm) 8.85 − 8.63 (m, 1H), 8.27 − 8.10 (m, 1H), 7.96 − 7.86 (m, 2H), 7.54 (d, J = 8.5 Hz, 1H), 7.40 − 7.27 (m, 4H), 7.25 − 7.14 (m, 1H), 5.62 (dd, J = 45.7, 7.6 Hz, 1H), 4.85 − 4.66 (m, 1H), 4.54 − 4.27 (m, 2H), 4.19 − 4.00 (m, 1H), 3.77 − 3.67 (m, 1H), 3.60 − 3.48 |
| | | | (m, 1H), 3.44 − 3.39 |
| | | | (m, 2H), 3.24 − 3.15 |
| | | | (m, 2H), 2.42 − 2.17 |
| | | | (m, 6H), 2.13 − 1.46 |
| | | | (m, 15H), 1.22 − |
| | | | 0.94 (m, 3H), 0.57 − |
| | | | 0.29 (m, 2H); 864.4 |
| | | | [M + H]⁺. |
| B6 | (((R)-(2-(((3S,6S,9S,10a R)-9-((3,3-difluoropropyl) (methyl)amino)- 5-oxo-3-((S)-6-phenyl-4- azaspiro[2.4]heptane-4- carbonyl)decahydropyrrolo [1,2-a]azocin-6-yl) carbamoyl)benzo[b] thiophen-5-yl) fluoromethyl)(hydroxy) phosphoryl)-L-alanine | 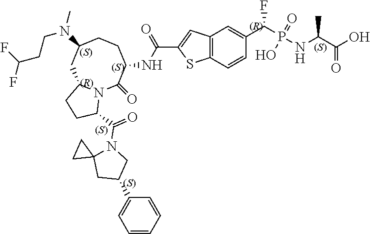 | 1H NMR (400 MHz, DMSO-d6) δ (ppm) 8.70 (d, J = 7.4 Hz, 1H), 8.20 (s, 1H), 7.92 (d, J = 8.1 Hz, 2H), 7.54 (d, J = 8.5 Hz, 1H), 7.38 − 7.28 (m, 4H), 7.22 (t, J = 7.1 Hz, 1H), 6.10 (t, J = 57.2 Hz, 1H), 5.61 (dd, J = 45.7, 7.6 Hz, 1H), 4.81 − 4.69 (m, 1H), 4.49 (t, J = 8.3 Hz, 1H), 4.34 (s, 1H), |
| | | | 4.10 (t, J = 8.6 Hz, |
| | | | 1H), 3.74 (t, J = 9.8 |
| | | | Hz, 1H), 3.58 − 3.37 |
| | | | (m, 2H), 3.28 − 2.98 |
| | | | (m, 2H), 2.45 − 2.30 |
| | | | (m, 2H), 2.29 − 2.15 |
| | | | (m, 3H), 2.14 − 2.05 |
| | | | (m, 2H), 2.04 − 1.93 |
| | | | (m, 4H), 1.92 − 1.35 |
| | | | (m, 9H), 1.06 (d, J = |
| | | | 6.8 Hz, 3H), 0.53 − |
| | | | 0.41 (m, 2H); 832.5 |
| | | | [M + H]⁺. |
| B7 | (((R)-fluoro(2-(((3S,6S,9S, 10aR)-9-(((1s,3R)-3- fluorocyclobutyl) (methyl)amino)- 5-oxo-3-((S)-6-phenyl-4- azaspiro[2.4]heptane-4- carbonyl) decahydropyrrolo[1,2- a]azocin-6-yl)carbamoyl) benzo[b]thiophen-5- yl)methyl)(hydroxy) phosphoryl)- L-alanine | 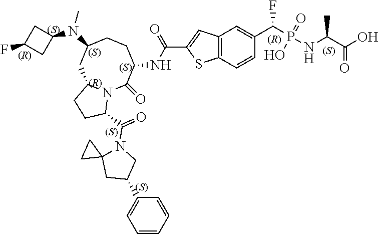 | 1H NMR (400 MHz, DMSO-d6) δ (ppm) 8.77 (s, 1H), 8.22 (s, 1H), 7.92 (d, J = 7.7 Hz, 2H), 7.56 (d, J = 8.2 Hz, 1H), 7.33 (dd, J = 12.2, 7.3 Hz, 4H), 7.22 (t, J = 7.0 Hz, 1H), 7.14 (t, J = 7.3 Hz, 1H), 6.76 (t, J = 7.2 Hz, 1H), 5.63 (dd, J = 45.7, 7.4 Hz, 1H), 4.99 − 4.64 (m, 2H), 4.57 − 4.33 |
| | | | (m, 2H), 4.16 − 4.06 |
| | | | (m, 1H), 3.74 (t, |
| | | | 1H), 3.61 − 3.45 (m, |
| | | | 2H), 2.80 − 2.56 (m, |
| | | | 2H), 2.47 − 1.39 (m, |
| | | | 21H), 1.09 (d, J = |
| | | | 6.7 Hz, 3H), 0.57 − |
| | | | 0.42 (m, 2H); 826.5 |
| | | | [M + H]⁺. |
| B8 | (((S)-(2-(((3S,6S,9S,10a R)-9-(ethyl((1s,3R)-3- fluorocyclobutyl)amino)- 5-oxo-3-((S)-6-phenyl-4- azaspiro[2.4]heptane-4- carbonyl)decahydropyrrolo [1,2-a]azocin-6- yl)carbamoyl)benzo[b] thiophen-5-yl) fluoromethyl)(hydroxy) phosphoryl)-L-alanine | 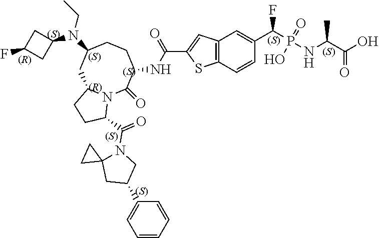 | 1H NMR (400 MHz, MeOD-d4) δ (ppm) 8.82 (s, 1H), 8.23 (s, 1H), 8.05 − 7.80 (m, 2H), 7.54 (d, J = 8.5 Hz, 1H), 7.38 − 7.26 (m, 4H), 7.25 − 7.19 (m, 1H), 5.64 (dd, J = 45.4, 7.3 Hz, 1H), 4.97 − 4.62 (m, 2H), 4.57 − 4.30 (m, 2H), 4.17 − 4.06 (m, 1H), 3.77 − 3.71 (m, 1H), 3.53 − 3.46 (m, 2H), 3.04 − |
| | | | 2.91 (m, 1H), 2.85 − |
| | | | 2.55 (m, 3H), 2.43 − |
| | | | 2.36 (m, 1H), 2.27 − |
| | | | 2.20 (m, 1H), 2.16 − |
| | | | 1.62 (m, 11H), 1.59 − |
| | | | 1.51 (m, 1H), 1.35 − |
| | | | 1.01 (m, 6H), 0.60 − |
| | | | 0.39 (m, 2H); |
| | | | 840.4 [M + H]⁺. |
| B9 | (((R)-fluoro(2-(((3S,6S,9S, 10aR)-9-(methyl((3- methyloxetan-3-yl)methyl) amino)-5-oxo-3-((S)-6- phenyl-4-azaspiro[2.4] heptane-4-carbonyl) decahydropyrrolo[1,2- a]azocin-6-yl)carbamoyl) benzo[b]thiophen-5- yl)methyl)(hydroxy) phosphoryl)- L-alanine | 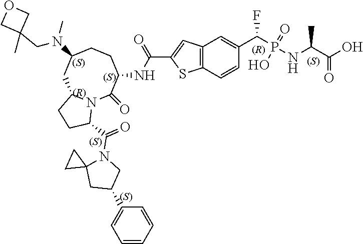 | 1H NMR (400 MHz, DMSO-d6) δ (ppm) 8.72 (d, J = 7.2 Hz, 1H), 8.20 (s, 1H), 7.93 (d, J = 8.1 Hz, 2H), 7.54 (d, J = 8.6 Hz, 1H), 7.39 − 7.27 (m, 4H), 7.26 − 7.08 (m, 2H), 5.72 − 5.63 (dd, J = 45.7, 7.6 Hz, 1H), 4.80 − 4.71 (m, 1H), 4.55 − 4.47 (m, 1H), 4.44 − 4.36 (m, 2H), 4.25 − 4.15 (m, 2H), 4.12 − |
| | | | 4.05 (m, 1H), 3.76 − |
| | | | 3.71 (m, 1H), 3.55 − |
| | | | 3.47 (m, 1H), 3.46 − |
| | | | 3.27 (m, 6H), 2.49 − |
| | | | 1.26 (m, 21H), 1.14 − |
| | | | 1.02 (m, 3H), 0.55 − |
| | | | 0.41 (m, 2H); |
| | | | 838.5 [M + H]⁺. |
| B10 | (((S)-fluoro(2-(((3S,6S,9S, 10aR)-9-(methyl((1s,3R)-3- (trifluoromethyl) cyclobutyl)amino)-5- oxo-3-((S)-6-phenyl-4- azaspiro[2.4]heptane-4- carbonyl) decahydropyrrolo[1,2- a]azocin-6- yl)carbamoyl)benzo[b] thiophen-5-yl)methyl) (hydroxy)phosphoryl)- L-alanine | 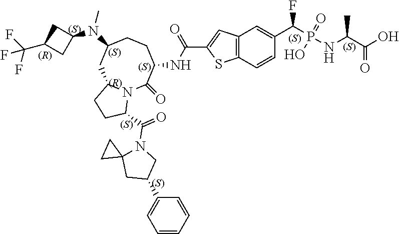 | 1H NMR (400 MHz, DMSO-d6) δ (ppm) 8.78 (s, 1H), 8.23 (s, 1H), 7.93 (d, J = 8.9 Hz, 2H), 7.53 (d, J = 8.5 Hz, 1H), 7.37 − 7.28 (m, 4H), 7.24 − 7.00 (m, 2H), 5.63 (dd, J = 45.5, 7.4 Hz, 1H), 4.72 (s, 1H), 4.58 − 4.28 (m, 2H), 4.17 − 4.06 (m, 1H), 3.81 − 3.39 (m, 5H), 2.96 − 2.78 (m, 1H), 2.45 − |
| | | | 1.45 (m, 21H), 1.10 |
| | | | (d, J = 6.8 Hz, 3H), |
| | | | 0.58 − 0.36 (m, 2H); |
| | | | 876.4 [M + H]⁺. |
| B11 | (((R)-(2-(((3S,6S,9S,10a R)-9-(((1s,3R)-3- cyanocyclobutyl) (methyl)amino)- 5-oxo-3-((S)-6- phenyl-4-azaspiro[2.4] heptane-4-carbonyl) decahydropyrrolo[1,2- a]azocin-6-yl)carbamoyl) benzo[b]thiophen-5- yl)fluoromethyl) (hydroxy)phosphoryl)- L-alanine | 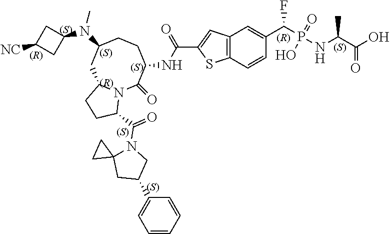 | 1H NMR (400 MHz, DMSO-d6) δ (ppm) 8.78 (s, 1H), 8.21 (s, 1H), 7.92 (d, J = 8.6 Hz, 2H), 7.56 (d, J = 8.5 Hz, 1H), 7.38 − 7.28 (m, 4H), 7.26 − 7.02 (m, 2H), 5.64 (dd, J = 45.7, 7.5 Hz, 1H), 4.70 (s, 1H), 4.57 − 4.32 (m, 2H), 4.12 (t, J = 8.3 Hz, 1H), 3.78 − 3.70 (m, 1H), 3.59 − 3.44 (m, 2H), |
| | | | 3.28 − 3.22 (m, 1H), |
| | | | 3.11 − 3.00 (m, 1H), |
| | | | 2.69 − 2.58 (m, 1H), |
| | | | 2.50 − 1.39 (m, |
| | | | 21H), 1.15 − 1.02 |
| | | | (m, 3H), 0.59 − 0.42 |
| | | | (m, 2H); 833.6 |
| | | | [M + H]⁺. |
| B12 | (((S)-(2-(((3S,6S,9S,10a R)-9-(((S)-3,3- difluorocyclopentyl) (methyl)amino)-5-oxo-3- ((S)-6-phenyl-4- azaspiro[2.4]heptane-4- carbonyl) decahydropyrrolo[1,2- a]azocin-6-yl)carbamoyl) benzo[b]thiophen-5- yl)fluoromethyl) (hydroxy)phosphoryl)- L-alanine | 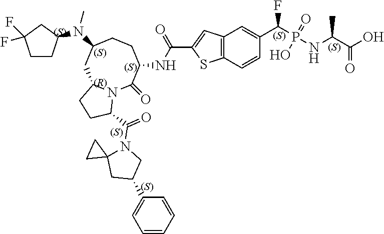 | 1H NMR (400 MHz, DMSO-d6) δ (ppm) 8.85 − 8.61 (m, 1H), 8.22 (s, 1H), 8.05 − 7.83 (m, 2H), 7.53 (d, J = 8.5 Hz, 1H), 7.37 − 7.27 (m, 4H), 7.23 − 7.19 (m, 1H), 5.65 (dd, J = 45.5, 7.3 Hz, 1H), 4.76 − 4.44 (m, 2H), 4.42 − 4.28 (m, 1H), 4.14 − 4.03 (m, 1H), 3.82 − 3.41 (m, 5H), 2.50 − 1.38 (m, |
| | | | 23H), 1.12 (d, J = |
| | | | 6.8 Hz, 3H), 0.57 − |
| | | | 0.35 (m, 2H); 858.4 |
| | | | [M + H]⁺. |
| B13 | ((R)-((S)-fluoro(2- (((3S,6S,9S,10a R)-9-(((1s,3R)-3- fluorocyclobutyl) (methyl)amino)- 5-oxo-3-((S)-6-phenyl-4- azaspiro[2.4]heptane-4- carbonyl) decahydropyrrolo[1,2- a]azocin-6- yl)carbamoyl)benzo[b] thiophen-6- yl)methyl)(hydroxy) phosphoryl)-L-alanine | 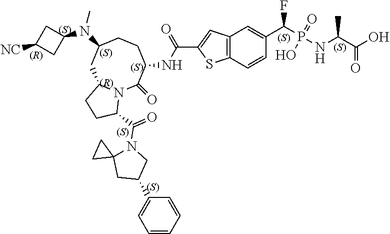 | 1H NMR (400 MHz, MeOD-d4) δ (ppm) 8.05 (s, 2H), 7.86 (d, J = 8.4 Hz, 1H), 7.60 (d, J = 8.5 Hz, 1H), 7.36 − 7.26 (m, 4H), 7.20 (t, J = 7.0 Hz, 1H), 7.17 − 6.74 (m, 1H), 5.66 (dd, J = 45.3, 7.0 Hz, 1H), 4.79 − 4.55 (m, 3H), 4.48 − 4.39 (m, 1H), 4.00 (t, J = 8.7 Hz, 1H), 3.94 − 3.75 (m, 3H), 3.59 − |
| | | | 3.48 (m, 1H), 3.39 − |
| | | | 3.32 (m, 1H), 3.01 − |
| | | | 2.90 (m, 1H), 2.80 − |
| | | | 2.70 (m, 1H), 2.69 − |
| | | | 2.57 (m, 1H), 2.55 |
| | | | (s, 3H), 2.50 − 2.37 |
| | | | (m, 2H), 2.31 (t, J = |
| | | | 11.5 Hz, 1H), 2.22 − |
| | | | 2.11 (m, 2H), 2.06 |
| | | | (d, J = 12.0 Hz, 1H), |
| | | | 2.01 − 1.69 (m, 8H), |
| | | | 1.64 − 1.56 (m, 1H), |
| | | | 1.32 (d, J = 7.0 Hz, |
| | | | 3H), 0.57 − 0.43 (m, |
| | | | 3H); 833.7 [M + H]⁺. |
| B14 | (((S)-fluoro(2-(((3S,6S,9S, 10aR)-9-(methyl(4,4,4- trifluorobutyl)amino)-5- oxo-3-((S)-6-phenyl-4- azaspiro[2.4]heptane-4- carbonyl)decahydropyrrolo [1,2-a]azocin-6-yl) carbamoyl)benzo[b] thiophen-5- yl)methyl)(hydroxy) phosphoryl)- L-alanine | 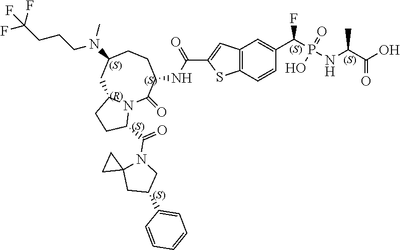 | 1H NMR (400 MHz, MeOD-d4) δ (ppm) 8.11 − 8.04 (m, 2H), 7.90 (d, J = 8.5 Hz, 1H), 7.61 (d, J = 8.5 Hz, 1H), 7.39 − 7.28 (m, 4H), 7.26 − 7.20 (m, 1H), 5.67 (dd, J = 45.3, 7.0 Hz, 1H), 4.71 − 4.58 (m, 2H), 4.46 − 4.35 (m, 1H), 4.06 − 3.76 (m, 4H), 3.61 − 3.52 (m, 1H), 3.21 − 3.11 (m, 2H), 2.78 (s, 3H), 2.52 − 2.41 (m, 1H), 2.37 − |
| | | | 2.27 (m, 3H), 2.20 − |
| | | | 1.81 (m, 13H), 1.66 − |
| | | | 1.57 (m, 1H), 1.33 |
| | | | (d, J = 7.0 Hz, 3H), |
| | | | 0.58 − 0.39 (m, 2H); |
| | | | 864.6 |
| | | | [M + H]⁺. |
| B15 | (((R)-fluoro(2(((3S,6S,9S, 10aR)-9-(((1s,3R)-3- fluorocyclobutyl) (methyl)amino)-5-oxo-3- (4-azaspiro[2.4]heptane-4- carbonyl)decahydropyrrolo [1,2-a]azocin-6- yl)carbamoyl)benzo[b] thiophen-5-yl)methyl) (hydroxy)phosphoryl)- L-alanine | 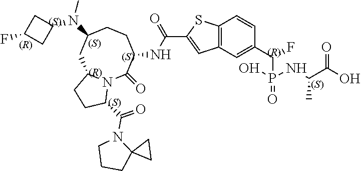 | 1H NMR (400 MHz, MeOD-d4) δ (ppm) 8.07 − 7.98 (m, 2H), 7.82 (d, J = 8.5 Hz, 1H), 7.64 (d, J = 8.5 Hz, 1H), 5.61 (dd, J = 45.3, 7.2 Hz, 1H), 4.79 − 4.55 (m, 3H), 4.43 − 4.35 (m, 1H), 4.00 − 3.92 (m, 1H), 3.75 − |
| | | | 3.56 (m, 2H), 3.27 − |
| | | | 3.21 (m, 1H), 2.73 |
| | | | 2.59 (m, 2H), 2.56 |
| | | | 2.46 (m, 1H), 2.42 |
| | | | 2.33 (m, 1H), 2.27 − |
| | | | 2.11 (m, 3H), 2.07 |
| | | | (s, 3H), 2.02 − 1.60 |
| | | | (m, 14H), 1.30 (d, J = |
| | | | 6.9 Hz, 3H), 0.51 − |
| | | | 0.39 (m, 2H); |
| | | | 750.6 [M + H]⁺. |
| B16 | (((S)-(2-(((3S,6S,9S,10a R)-9-(ethyl((1s,3R)-3- (trifluoromethoxy) cyclobutyl)amino)-5-oxo- 3-((S)-6-phenyl-4- azaspiro[2.4]heptane-4- carbonyl)decahydropyrrolo [1,2-a]azocin-6-yl) carbamoyl)benzo[b] thiophen-5- yl)fluoromethyl) (hydroxy)phosphoryl)- L-alanine | 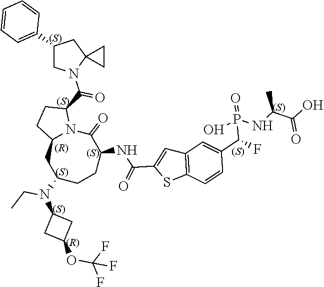 | 1H NMR (400 MHz, MeOD-d4) δ (ppm) 8.05 (d, J = 3.1 Hz, 2H), 7.86 (d, J = 8.4 Hz, 1H), 7.60 (d, J = 8.4 Hz, 1H), 7.37 − 7.25 (m, 4H), 7.20 (t, J = 7.1 Hz, 1H), 5.65 (dd, J = 45.3, 7.0 Hz, 1H), 4.68 − 4.52 (m, 3H), 4.48 − 4.38 (m, 1H), 4.01 (t, J = 8.8 Hz, 1H), 3.93 − 3.73 (m, 3H), 3.59 − 3.48 (m, 2H), 3.17 − 3.07 (m, 1H), 3.06 − 2.95 (m, 2H), 2.84 − 2.54 (m, |
| | | | 3H), 2.48 − 2.39 (m, |
| | | | 1H), 2.32 (t, J = 11.5 |
| | | | Hz, 1H), 2.23 − 2.11 |
| | | | (m, 2H), 2.07 − 1.81 |
| | | | (m, 8H), 1.78 − 1.67 |
| | | | (m, 1H), 1.62 − 1.54 |
| | | | (m, 1H), 1.36 − 1.24 |
| | | | (m, 6H), 0.58 − 0.42 |
| | | | (m, 2H); 906.4 |
| | | | [M + H]⁺. |
| B17 | (((S)-(2-(((3S,6S,9S,10a R)-9-(ethyl(4,4,4- trifluorobutyl)amino)-5- oxo-3-((S)-6-phenyl-4- azaspiro[2.4]heptane-4- carbonyl) decahydropyrrolo[1,2-a] azocin-6-yl)carbamoyl) benzo[b]thiophen-5- yl)fluoromethyl) (hydroxy)phosphoryl)- L-alanine | 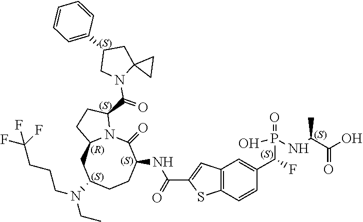 | 1H NMR (400 MHz, MeOD-d4) δ (ppm) 8.06 − 8.00 (m, 2H), 7.81 (d, J = 8.5 Hz, 1H), 7.61 (d, J = 8.5 Hz, 1H), 7.39 − 7.34 (m, 2H), 7.29 (t, J = 7.6 Hz, 2H), 7.22 − 7.17 (m, 1H), 5.61 (dd, J = 45.4, 7.2 Hz, 1H), 4.78 − 4.72 (m, 1H), 4.59 (t, J = 8.7 Hz, 1H), 4.45 − 4.34 (m, |
| | | | 1H), 4.07 − 3.91 (m, |
| | | | 2H), 3.71 − 3.50 (m, |
| | | | 2H), 3.25 − 3.17 (m, |
| | | | 1H), 2.61 − 2.09 (m, |
| | | | 12H), 2.04 − 1.73 |
| | | | (m, 9H), 1.65 − 1.55 |
| | | | (m, 2H), 1.32 (d, J = |
| | | | 6.9 Hz, 3H), 1.07 (t, |
| | | | J = 7.1 Hz, 3H), |
| | | | 0.54 − 0.43 (m, 2H); |
| | | | 878.6 |
| | | | [M + H]⁺. |
| B18 | ((S)-((R)-(2-(((3S,6S,9S, 10aR)-9-(((1s,3R)-3- (difluoromethyl) cyclobutyl)(methyl)amino)- 5-oxo-3-((S)-6-phenyl-4- azaspiro[2.4]heptane-4- carbonyl)decahydropyrrolo [1,2-a]azocin-6-yl) carbamoyl)benzo[b] thiophen-5-yl) fluoromethyl)(hydroxy) phosphoryl)-L-alanine | 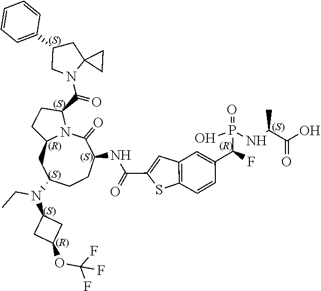 | 1H NMR (400 MHz, MeOD-d4) δ (ppm) 8.10 − 7.99 (m, 2H), 7.89 (d, J = 8.8 Hz, 1H), 7.66 (d, J = 9.8 Hz, 1H), 7.34 − 7.25 (m, 4H), 7.19 (t, J = 7.0 Hz, 1H), 6.07 − 5.58 (m, 2H), 4.64 4.54 (m, 2H), 4.44 − 4.35 (m, 1H), 3.98 (t, J = 8.7 Hz, 1H), 3.91 − 3.65 (m, 4H), 3.55 − 3.45 (m, 1H), 2.62 − 2.05 (m, 13H), 1.99 − 1.66 (m, 9H), 1.62 − 1.55 (m, 1H), 1.33 (d, J = 7.0 Hz, |
| | | | 3H), 0.55 − 0.40 (m, |
| | | | 2H); 858.6 |
| | | | [M + H]⁺. |
| B19 | (((R)-fluoro(2-(((3S,6S,9S, 10aR)-9-(methyl((R)- tetrahydrofuran- 3-yl)amino)-5-oxo-3-((S)-6- phenyl-4-azaspiro[2.4] heptane-4-carbonyl) decahydropyrrolo[1,2-a] azocin-6-yl)carbamoyl) benzo[b]thiophen-5- yl)methyl)(hydroxy) phosphoryl)-L-alanine | 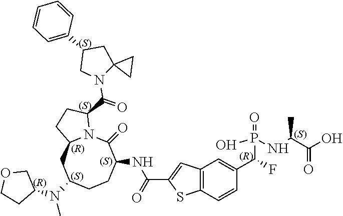 | 1H NMR (400 MHz, MeOD-d4) δ (ppm) 7.92 (s, 1H), 7.86 (s, 1H), 7.77 (d, J = 8.5 Hz, 1H), 7.53 (d, J = 8.5 Hz, 1H), 7.22 − 7.12 (m, 4H), 7.09 − 7.03 (m, 1H), 5.52 (dd, J = 45.3, 7.0 Hz, 1H), 4.45 (t, J = 8.3 Hz, 1H), 4.27 (d, J = 11.7 Hz, 1H), 4.14 − 3.99 (m, 2H), 3.94 3.84 (m, 3H), 3.79 |
| | | | 3.66 (m, 3H), 3.61 − |
| | | | 3.51 (m, 2H), 3.45 − |
| | | | 3.36 (m, 1H), 2.53 |
| | | | (s, 3H), 2.37 − 2.26 |
| | | | (m, 2H), 2.17 (t, J = |
| | | | 11.5 Hz, 1H), 2.09 − |
| | | | 1.68 (m, 9H), 1.64 − |
| | | | 1.51 (m, 2H), 1.49 |
| | | | 1.41 (m, 1H), 1.37 |
| | | | 1.27 (m, 1H), 1.21 |
| | | | (d, J = 7.0 Hz, 3H), |
| | | | 0.41 − 0.31 (m, 2H); |
| | | | 824.6 |
| | | | [M + H]⁺. |
| B20 | (((S)-fluoro(2-(((3S,6S,9S, 10aR)-9-(methyl(((R)- oxetan-2-yl)methyl)amino)- 5-oxo-3-((S)-6-phenyl-4- azaspiro[2.4]heptane-4- carbonyl)decahydropyrrolo [1,2-a]azocin-6- yl)carbamoyl)benzo[b] thiophen-5-yl)methyl) (hydroxy)phosphoryl)- L-alanine | 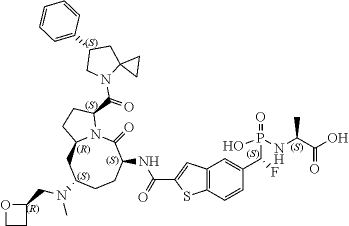 | 1H NMR (400 MHz, MeOD-d4) δ (ppm) 8.05 (d, J = 6.0 Hz, 2H), 7.84 (d, J = 8.4 Hz, 1H), 7.60 (d, J = 8.5 Hz, 1H), 7.36 − 7.25 (m, 4H), 7.23 − 7.16 (m, 1H), 5.62 (dd, J = 45.4, 7.1 Hz, 1H), 5.23 − 5.10 (m, 1H), 4.72 − 4.52 (m, 4H), 4.42 − 4.33 (m, 1H), 4.06 − 3.86 (m, 2H), |
| | | | 3.74 − 3.65 (m, 1H), |
| | | | 3.60 − 3.47 (m, 2H), |
| | | | 3.27 − 3.18 (m, 1H), |
| | | | 3.11 − 3.01 (m, 1H), |
| | | | 2.84 − 2.71 (m, 1H), |
| | | | 2.55 (s, 3H), 2.47 − |
| | | | 2.36 (m, 2H), 2.33 − |
| | | | 1.93 (m, 8H), 1.91 − |
| | | | 1.60 (m, 6H), 1.31 |
| | | | (d, J = 7.0 Hz, 3H), |
| | | | 0.54 − 0.40 (m, 2H); |
| | | | 824.3 [M + H]⁺. |
| B21 | (((S)-fluoro(2-(((3S,6S,9S, 10aR)-9-(methyl(oxetan- 3-yl)amino)-5-oxo-3-((S)-6- phenyl-4-azaspiro[2.4] heptane-4-carbonyl) decahydropyrrolo[1,2-a] azocin-6-yl)carbamoyl) benzo[b]thiophen-5- yl)methyl)(hydroxy) phosphoryl)-L-alanine | 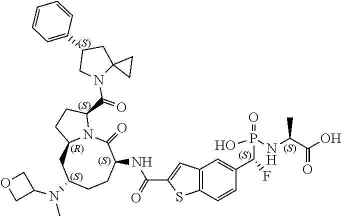 | 1H NMR (400 MHz, MeOD-d4) δ (ppm) 8.06 − 8.01 (m, 2H), 7.85 − 7.80 (m, 1H), 7.62 (d, J = 8.3 Hz, 1H), 7.38 − 7.27 (m, 4H), 7.20 (t, J = 7.2 Hz, 1H), 5.61 (dd, J = 45.4, 7.1 Hz, 1H), 4.81 − 4.71 (m, 4H), 4.66 − 4.55 (m, 3H), 4.45 − 4.36 (m, 1H), 4.04 (t, J = 8.7 Hz, |
| | | | 1H), 3.99 − 3.89 (m, |
| | | | 2H), 3.71 − 3.64 (m, |
| | | | 1H), 3.61 − 3.50 (m, |
| | | | 1H), 3.14 − 3.04 (m, |
| | | | 1H), 2.44 − 2.32 (m, |
| | | | 2H), 2.28 − 2.10 (m, |
| | | | 6H), 2.01 − 1.93 (m, |
| | | | 3H), 1.89 − 1.72 (m, |
| | | | 4H), 1.69 − 1.61 (m, |
| | | | 2H), 1.31 (d, J = 6.8 |
| | | | Hz, 3H), 0.55 − 0.47 |
| | | | (m, 2H); 810.4 |
| | | | [M + H]⁺. |
| B22 | (((R)-fluoro(2-(((3S,6S,9S, 10aR)-9-(methyl(((R)- oxetan-2-yl)methyl)amino)- 5-oxo-3-((S)-6-phenyl-4- azaspiro[2.4]heptane-4- carbonyl)decahydropyrrolo [1,2-a]azocin-6-yl) carbamoyl)benzo[b] thiophen-5- yl)methyl)(hydroxy) phosphoryl)- L-alanine | 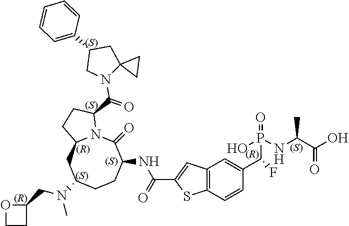 | 1H NMR (400 MHz, MeOD-d4) δ (ppm) 8.08 − 7.99 (m, 2H), 7.88 − 7.79 (m, 1H), 7.67 − 7.61 (m, 1H), 7.39 − 7.26 (m, 4H), 7.20 (t, J = 7.2 Hz, 1H), 5.63 (dd, J = 45.3, 7.3 Hz, 1H), 5.19 − 5.10 (m, 1H), 4.73 − 4.53 (m, 4H), 4.46 − 4.35 (m, 1H), 4.04 (t, J = 8.7 Hz, 1H), 3.97 − |
| | | | 3.87 (m, 1H), 3.78 − |
| | | | 3.68 (m, 1H), 3.59 − |
| | | | 3.46 (m, 2H), 3.23 − |
| | | | 3.11 (m, 1H), 3.09 − |
| | | | 3.00 (m, 1H), 2.84 − |
| | | | 2.71 (m, 1H), 2.49 − |
| | | | 2.39 (m, 2H), 2.33 |
| | | | (t, J = 11.5 Hz, 1H), |
| | | | 2.28 − 2.21 (m, 1H), |
| | | | 2.20 − 1.93 (m, 7H), |
| | | | 1.93 − 1.77 (m, 5H), |
| | | | 1.75 − 1.61 (m, 2H), |
| | | | 1.36 − 1.24 (m, 3H), |
| | | | 0.57 − 0.45 (m, 2H); |
| | | | 824.4 [M + H]⁺. |
| B23 | (((R)-fluoro(2- (((3S,6S,9S,10aR)-9- (methyl(((S)-oxetan-2- yl)methyl)amino)- 5-oxo-3-((S)-6-phenyl-4- azaspiro[2.4]heptane-4- carbonyl)decahydropyrrolo [1,2-a]azocin-6- yl)carbamoyl) benzo[b]thiophen-5- yl)methyl)(hydroxy) phosphoryl)-L-alanine | 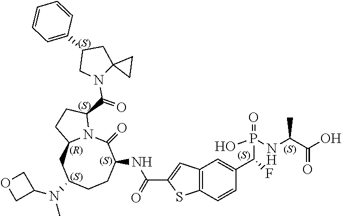 | 1H NMR (400 MHz, MeOD-d4) δ (ppm) 8.07 − 8.04 (m, 1H), 8.03 − 7.99 (m, 1H), 7.87 − 7.83 (m, 1H), 7.66 − 7.60 (m, 1H), 7.36 − 7.25 (m, 4H), 7.22 − 7.18 (m, 1H), 5.64 (dd, J = 45.3, 7.3 Hz, 1H), 5.22 − 5.14 (m, 1H), 4.71 − 4.62 (m, 2H), 4.62 − 4.53 (m, 2H), |
| | | | 4.43 − 4.34 (m, 1H), |
| | | | 4.06 − 3.99 (m, 1H), |
| | | | 3.95 − 3.87 (m, 1H), |
| | | | 3.80 − 3.70 (m, 1H), |
| | | | 3.63 − 3.47 (m, 2H), |
| | | | 3.30 − 3.24 (m, 1H), |
| | | | 3.15 − 3.07 (m, 1H), |
| | | | 2.84 − 2.73 (m, 1H), |
| | | | 2.48 − 2.37 (m, 2H), |
| | | | 2.36 − 2.28 (m, 1H), |
| | | | 2.25 − 2.13 (m, 2H), |
| | | | 2.10 − 1.92 (m, 5H), |
| | | | 1.91 − 1.78 (m, 3H), |
| | | | 1.75 − 1.60 (m, 2H), |
| | | | 1.35 − 1.24 (m, 3H), |
| | | | 0.58 − 0.41 (m, 2H); |
| | | | 824.4 [M + H]⁺. |
| B24 | (((S)-fluoro(2- (((3S,6S,9S,10a R)-9-morpholino-5-oxo-3- ((R)-6-(pyridin-3-yl)-4- azaspiro[2.4]heptane-4- carbonyl)decahydropyrrolo [1,2-a]azocin-6- yl)carbamoyl)benzo [b]thiophen-5-yl) methyl)(hydroxy) phosphoryl)-L-alanine | 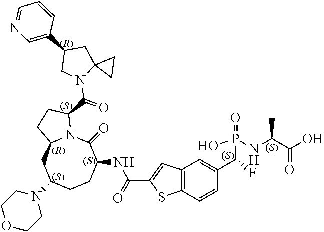 | 1H NMR (400 MHz, MeOD-d4) δ (ppm) 8.59 − 8.52 (m, 1H), 8.44 − 8.36 (m, 1H), 8.14 − 8.07 (m, 2H), 7.91 (t, J = 7.5 Hz, 2H), 7.63 − 7.55 (m, 1H), 7.42 − 7.35 (m, 1H), 5.68 (dd, J = 45.2, 6.6 Hz, 1H), 4.63 − 4.55 (m, 1H), 4.49 − 4.31 (m, 1H), 4.18 − 3.97 (m, 3H), 3.91 − 3.76 |
| | | | (m, 5H), 3.69 − 3.59 |
| | | | (m, 1H), 3.12 − 3.03 |
| | | | (m, 1H), 2.98 − 2.73 |
| | | | (m, 4H), 2.50 − 2.38 |
| | | | (m, 1H), 2.30 − 2.10 |
| | | | (m, 4H), 2.07 − 1.65 |
| | | | (m, 7H), 1.50 − 1.35 |
| | | | (m, 4H), 1.28 − 1.09 |
| | | | (m, 1H), 0.58 − 0.44 |
| | | | (m, 2H); 811.6 |
| | | | [M + H]⁺. |
| B25 | (((R)-fluoro(2-(((3S,6S,9S, 10aR)-9-(((1s,3R)-3- fluorocyclobutyl)(methyl) amino)-5-oxo-3-((S)-6- phenyl-4-azaspiro[2.4] heptane-4-carbonyl) decahydropyrrolo[1,2- a]azocin-6-yl)carbamoyl) benzo[b]thiophen-5- yl)methyl)(hydroxy) phosphoryl)-D-alanine | 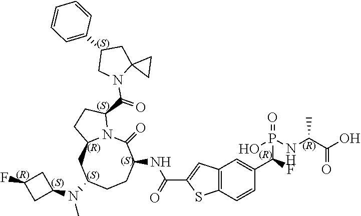 | 1H NMR (400 MHz, MeOD-d4) δ (ppm) 8.02 (d, J = 10.9 Hz, 2H), 7.82 (d, J = 8.5 Hz, 1H), 7.64 (d, J = 8.5 Hz, 1H), 7.39 − 7.26 (m, 4H), 7.20 (t, J = 7.2 Hz, 1H), 5.61 (dd, J = 45.4, 7.2 Hz, 1H), 4.78 − 4.64 (m, 2H), 4.60 (t, J = 8.7 Hz, 1H), 4.44 − 4.35 (m, 1H), 4.04 (t, J = 8.7 |
| | | | Hz, 1H), 3.93 (t, J = |
| | | | 9.8 Hz, 1H), 3.76 − |
| | | | 3.66 (m, 1H), 3.61 − |
| | | | 3.51 (m, 1H), 2.80 − |
| | | | 2.62 (m, 2H), 2.60 − |
| | | | 2.49 (m, 1H), 2.46 − |
| | | | 2.23 (m, 4H), 2.22 − |
| | | | 2.05 (m, 6H), 2.02 − |
| | | | 1.92 (m, 3H), 1.87 − |
| | | | 1.74 (m, 2H), 1.73 − |
| | | | 1.58 (m, 3H), 1.31 |
| | | | (d, J = 8.2 Hz, 3H), |
| | | | 0.55 − 0.46 (m, 2H); |
| | | | 827.5 [M + H]⁺. |
| B26 | (((R)-(2-(((3S,6S,9S,10a R)-9-(ethyl(4,4,4- trifluorobutyl)amino)-5- oxo-3-((S)-6-phenyl-4- azaspiro[2.4]heptane-4- carbonyl)decahydropyrrolo [1,2-a]azocin-6-yl) carbamoyl)benzo[b] thiophen-5-yl)fluoromethyl) (hydroxy)phosphoryl)- L-alanine | 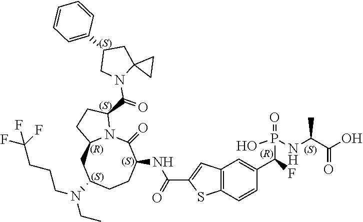 | 1H NMR (400 MHz, MeOD-d4) δ (ppm) 8.06 − 8.00 (m, 2H), 7.86 − 7.80 (m, 1H), 7.64 (d, J = 8.5 Hz, 1H), 7.38 − 7.26 (m, 4H), 7.20 (t, J = 7.2 Hz, 1H), 5.62 (dd, J = 45.3, 7.2 Hz, 1H), 4.73 (d, J = 10.6 Hz, 1H), 4.59 (t, J = 8.7 Hz, 1H), 4.43 − 4.34 (m, 1H), 4.04 (t, J = 8.7 |
| | | | Hz, 1H), 3.94 (t, J = |
| | | | 9.7 Hz, 1H), 3.75 − |
| | | | 3.67 (m, 1H), 3.59 − |
| | | | 3.49 (m, 1H), 3.40 − |
| | | | 3.32 (m, 1H), 2.73 − |
| | | | 2.50 (m, 4H), 2.49 − |
| | | | 2.31 (m, 2H), 2.29 − |
| | | | 2.11 (m, 5H), 2.06 − |
| | | | 1.91 (m, 4H), 1.87 − |
| | | | 1.74 (m, 5H), 1.69 − |
| | | | 1.59 (m, 2H), 1.31 |
| | | | (d, J = 6.9 Hz, 3H), |
| | | | 1.12 (t, J = 7.0 Hz, |
| | | | 3H), 0.53 − 0.45 (m, |
| | | | 2H); 878.4 [M + H]⁺. |
| B27 | (((R)-(2-(((3S,6S,9S,10a R)-9-(ethyl((1s,3R)-3- (trifluoromethoxy) cyclobutyl)amino)-5-oxo- 3-((S)-6-phenyl-4- azaspiro[2.4]heptane-4- carbonyl)decahydropyrrolo [1,2-a]azocin-6-yl)carbamoyl) benzo[b]thiophen-5- yl)fluoromethyl)(hydroxy) phosphoryl)-L-alanine | 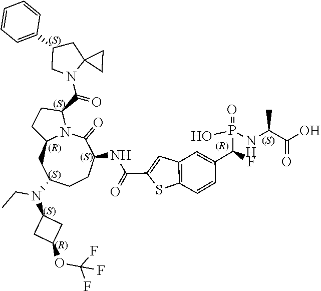 | 1H NMR (400 MHz, MeOD-d4) δ (ppm) 8.08 − 7.99 (m, 2H), 7.88 − 7.81 (m, 1H), 7.63 (d, J = 8.5 Hz, 1H), 7.38 − 7.26 (m, 4H), 7.22 − 7.17 (m, 1H), 5.65 (dd, J = 45.3, 7.2 Hz, 1H), 4.73 − 4.57 (m, 2H), 4.55 − 4.48 (m, 1H), 4.46 − 4.38 (m, 1H), 4.09 − 3.97 (m, 1H), 3.96 − 3.86 (m, 1H), 3.79 − 3.67 (m, 1H), 3.67 − 3.50 (m, 2H), 3.39 − 3.32 (m, 1H), 3.01 − 2.78 |
| | | | (m, 3H), 2.76 − 2.55 |
| | | | (m, 2H), 2.49 − 2.29 |
| | | | (m, 3H), 2.25 − 2.12 |
| | | | (m, 2H), 2.08 − 1.92 |
| | | | (m, 5H), 1.88 − 1.69 |
| | | | (m, 4H), 1.59 (s, |
| | | | 1H), 1.31 (d, J = 6.9 |
| | | | Hz, 3H), 1.27 − 1.21 |
| | | | (m, 3H), 0.50 (s, |
| | | | 2H); 906.4 [M + H]⁺. |
| B28 | (((R)-fluoro(2-(((3S,6S,9S, 10aR)-9-(methyl(oxetan- 3-yl)amino)-5-oxo-3-((S)-6- phenyl-4-azaspiro[ 2.4] heptane-4-carbonyl) decahdropyrrolo[1,2- a]azocin-6-yl)carbamoyl) benzo[b]thiophen-5- yl)methyl)(hydroxy) phosphoryl)-L-alanine | 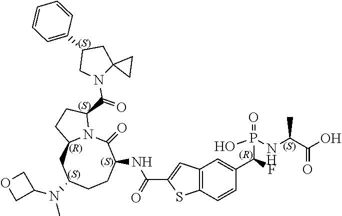 | 1H NMR (400 MHz, MeOD-d4) δ (ppm) 8.02 (d, J = 11.3 Hz, 2H), 7.82 (d, J = 8.5 Hz, 1H), 7.63 (d, J = 8.5 Hz, 1H), 7.36 (d, J = 7.3 Hz, 2H), 7.32 − 7.26 (m, 2H), 7.23 − 7.17 (m, 1H), 5.61 (dd, J = 45.3, 7.2 Hz, 1H), 4.79 − 4.70 (m, 3H), 4.65 − 4.56 (m, 3H), 4.43 − 4.34 (m, 1H), |
| | | | 4.07 − 4.01 (m, 1H), |
| | | | 3.98 − 3.88 (m, 2H), |
| | | | 3.74 − 3.65 (m, 1H), |
| | | | 3.63 − 3.52 (m, 1H), |
| | | | 3.12 − 3.04 (m, 1H), |
| | | | 2.46 − 2.13 (m, 5H), |
| | | | 2.10 (s, 3H), 2.03 − |
| | | | 1.91 (m, 3H), 1.87 − |
| | | | 1.70 (m, 4H), 1.69 − |
| | | | 1.59 (m, 2H), 1.30 |
| | | | (d, J = 6.9 Hz, 3H), |
| | | | 0.55 − 0.47 (m, 2H); |
| | | | 810.7 [M + H]⁺. |

TABLE 1C

| No. | Name | Structure |
|---|---|---|
| C1 | propyl (((R)-(2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 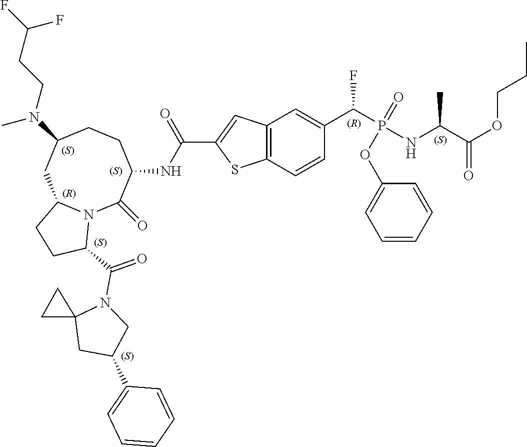 |
| C2 | propyl (((1R)-(2- (((3S,6S,9S,10aR)- 9-((3,3- difluorocyclobutyl) (methyl)amino)- 5-oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)fluoromethyl) (phenoxy) phosphoryl)- L-alaninate | 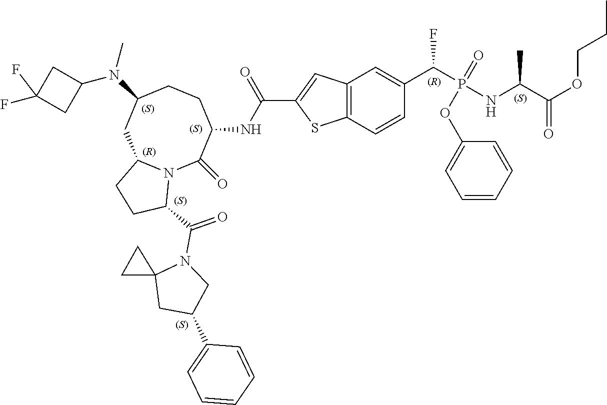 |
| C3 | propyl (((1S)-(2- (((3S,6S,9S,10aR)- 9-((3,3- difluorocyclobutyl) (methyl)amino)- 5-oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)fluoromethyl) (phenoxy) phosphoryl)- L-alaninate | 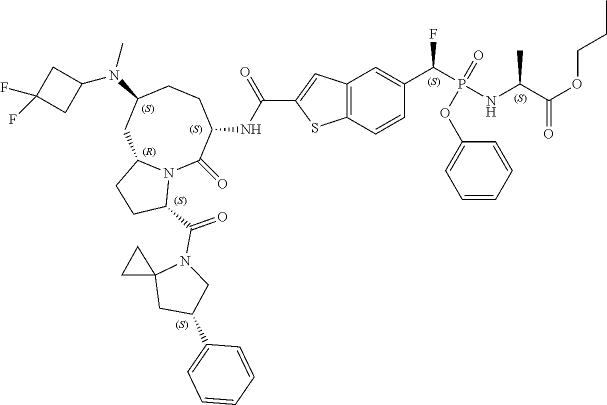 |
| C4 | propyl ((R)-((1R)- fluoro(2- (((3S,6S,9S,10aR)- 9- (methyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)methyl) (phenoxy) phosphoryl)-L- alaninate | 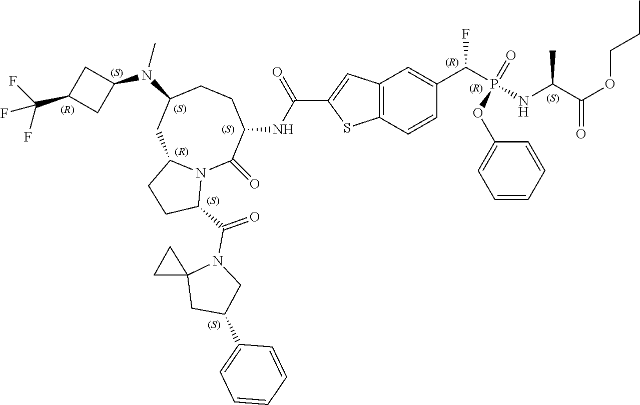 |
| C5 | propyl ((R)-((1R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro [2.4] heptane-4- carbonyl) decahydropyrrolo [1,2- a]azocin-6- yl)carbamoyl)- 2,3- dihydrobenzo[b] thiophen-5- yl)methyl) (phenoxy) phosphoryl)-L- alaninate | 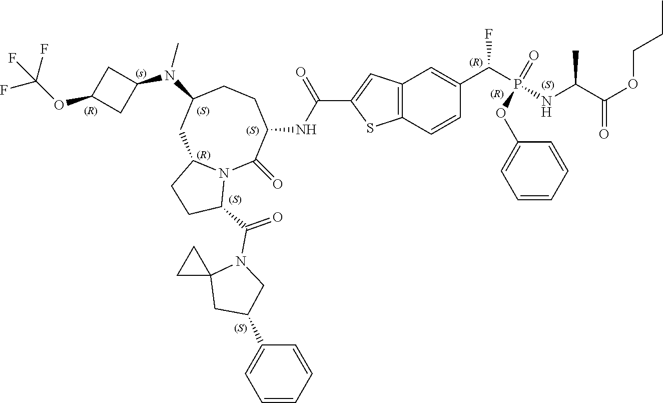 |
| C6 | propyl ((S)-((1R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)methyl) (phenoxy) phosphoryl)-L- alaninate | 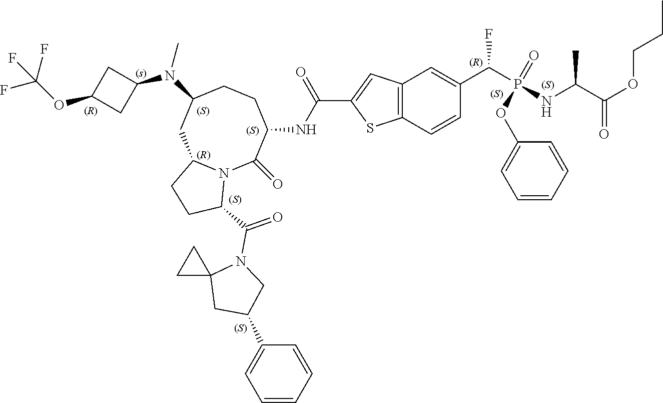 |
| C7 | propyl ((R)-((S)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 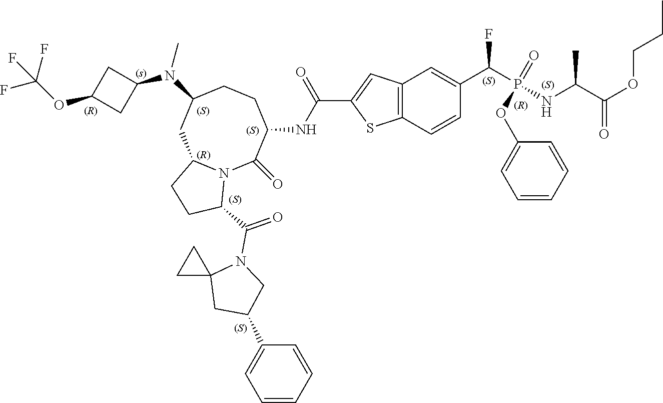 |
| C8 | propyl ((S)-((R)- fluoro(2- (((3S,6S,9S,10aR)- 9-(methyl(4,4,4- trifluorobutyl) amino)-5-oxo-3-((S)- 6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 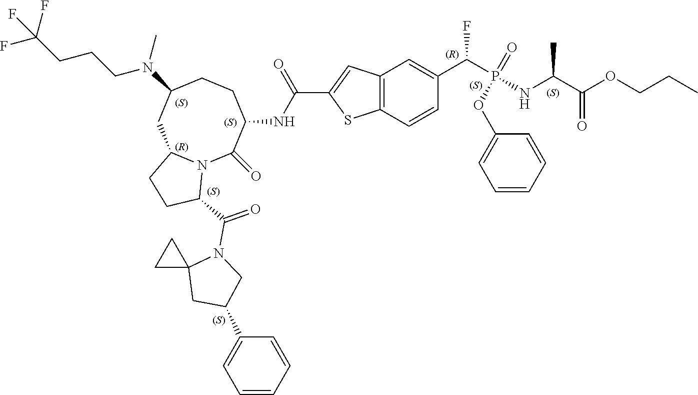 |
| C9 | propyl (((1S)-(2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)fluoromethyl) (phenoxy) phosphoryl)- L-alaninate | 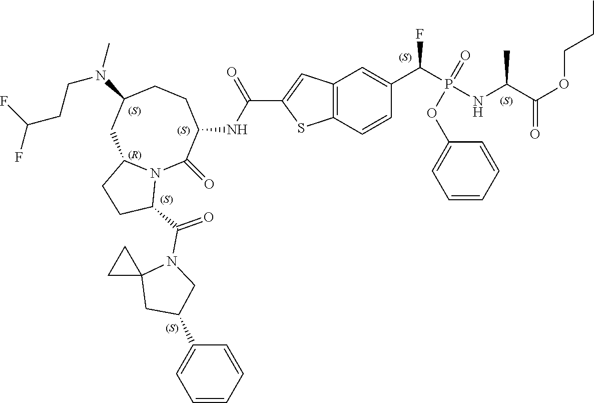 |
| C10 | propyl ((S)-((R)- fluoro(2- (((3S,6S,9S,10aR)- 9- (methyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 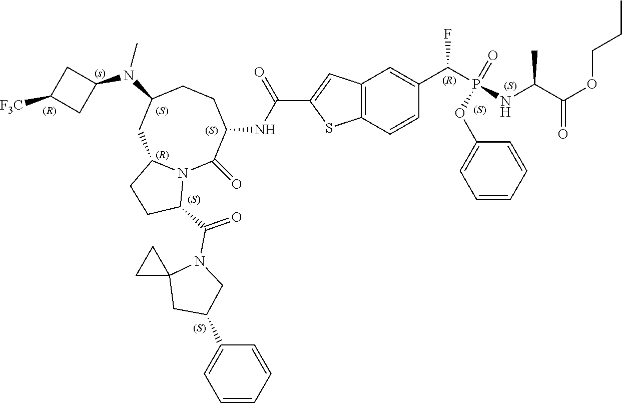 |
| C11 | propyl ((S)-((R)- (2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 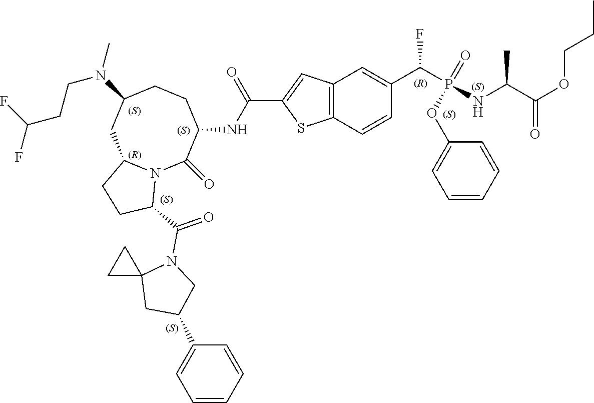 |
| C12 | propyl ((R)-((R)- (2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 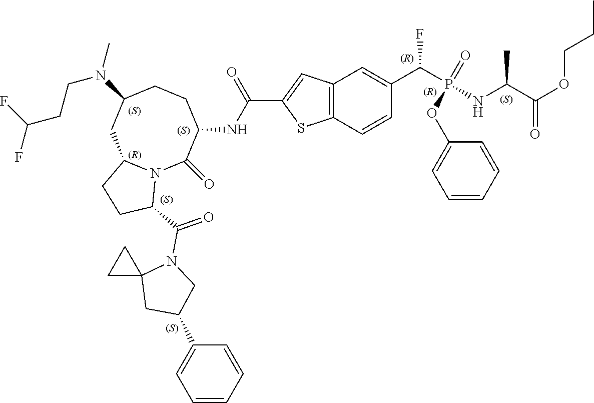 |
| C13 | propyl (((R)- fluoro(2- (((3S,6S,9S,10aR)- 9- (methyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 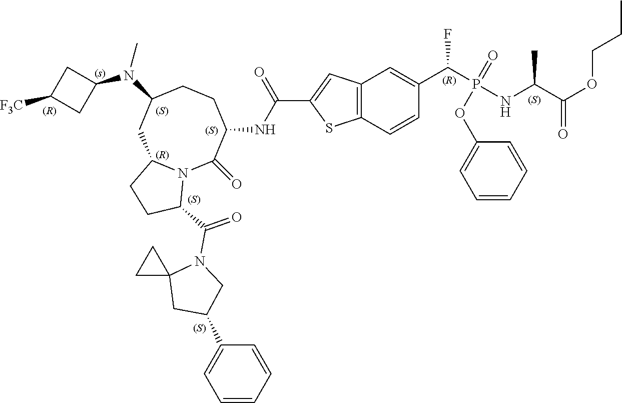 |
| C14 | cyclobutyl ((S)- ((S)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 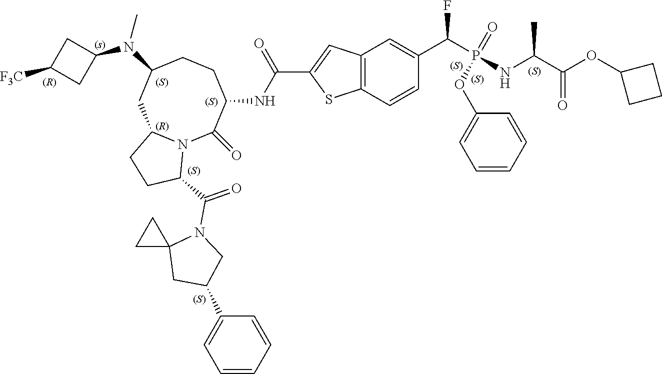 Phosphorus absolute stereochemistry arbitrarily assigned |
| C15 | cyclobutyl ((R)- ((S)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 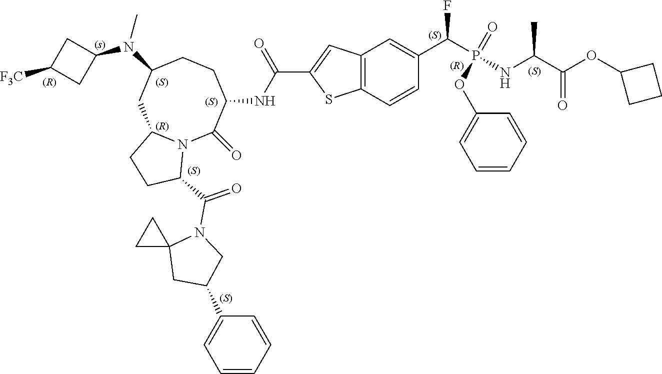 Phosphorus absolute stereochemistry arbitrarily assigned |
| C16 | propyl (((R)- fluoro(2- (((3S,6S,9S,10aR)- 9-(((1r,3S)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 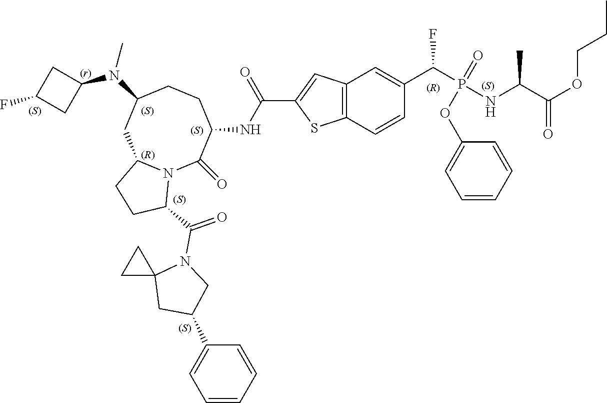 |
| C17 | propyl ((S)-((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 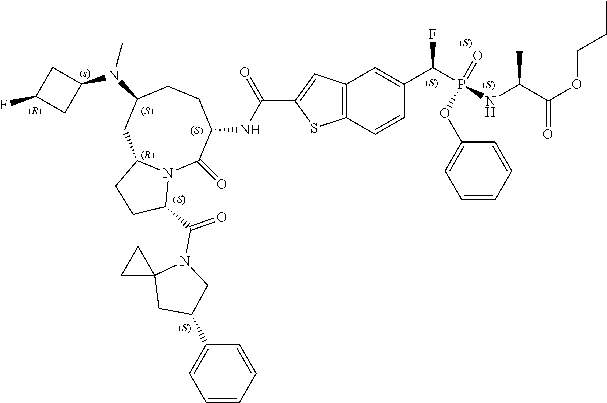 |
| C18 | propyl (((R)- fluoro(2- (((3S,6S,9S,10aR)- 9-(methyl(((S)- oxetan-2- yl)methyl)amino)- 5-oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 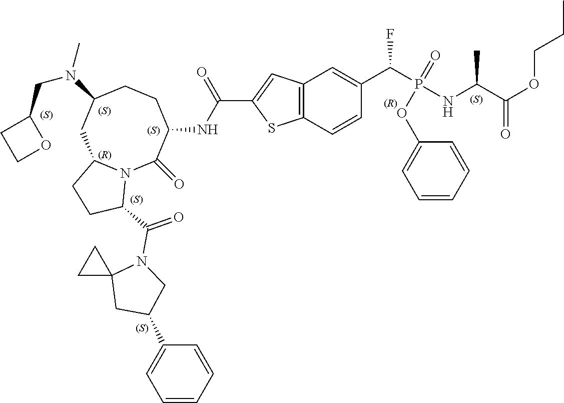 |
| C19 | propyl ((R)-((1R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)methyl) (phenoxy) phosphoryl)-L- alaninate | 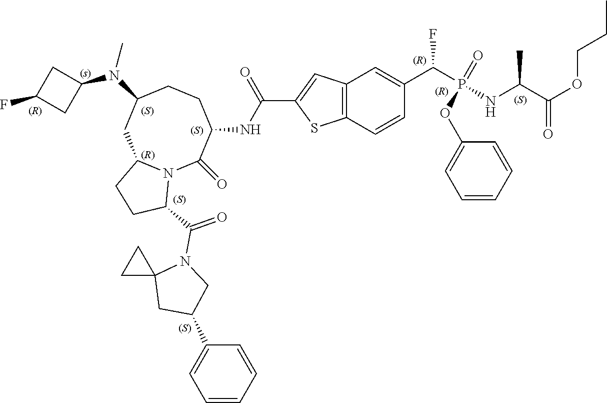 |
| C20 | propyl (((1R)- fluoro(2- (((3S,6S,9S,10aR)- 9-(methyl(((R)- oxetan-2- yl)methyl)amino)- 5-oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)methyl) (phenoxy) phosphoryl)-L- alaninate | 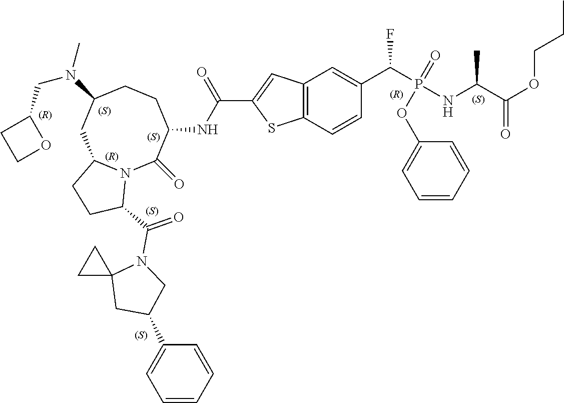 |
| C21 | propyl (((1S)- fluoro(2- (((3S,6S,9S,10aR)- 9-(methyl(((S)- oxetan-2- yl)methyl)amino)- 5-oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)methyl) (phenoxy) phosphoryl)-L- alaninate | 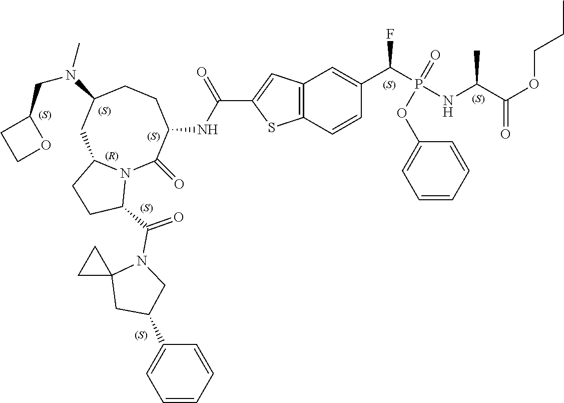 |
| C22 | propyl (((1S)- fluoro(2- (((3S,6S,9S,10aR)- 9-(methyl((R)- tetrahydrofuran-3- yl)amino)-5-oxo- 3-((S)-6-phenyl-4- azaspiro [2.4] heptane-4- carbonyl) decahydropyrrolo [1,2- a]azocin-6- yl)carbamoyl)- 2,3- dihydrobenzo[b] thiophen-5- yl)methyl) (phenoxy) phosphoryl)-L- alaninate | 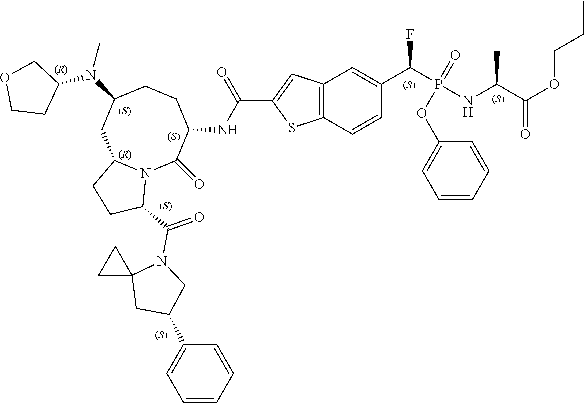 |
| C23 | propyl ((S)-((R)- fluoro(2- (((3S,6S,9S,10aR)- 9-(((1s,3R)-3- fluorocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 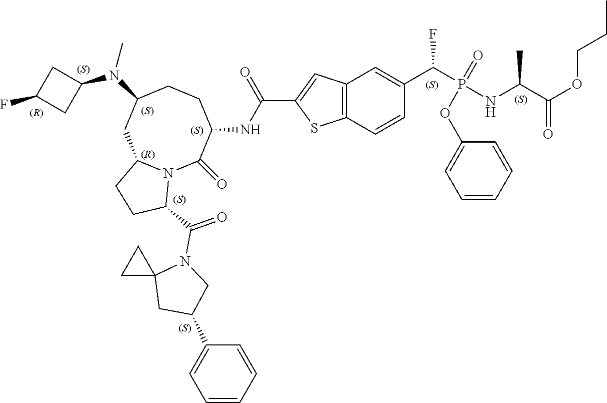 |
| C24 | propyl (((R)- fluoro(2- (((3S,6S,9S,10aR)- 9-(methyl((R)- tetrahydrofuran-3- yl)amino)-5-oxo- 3-((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 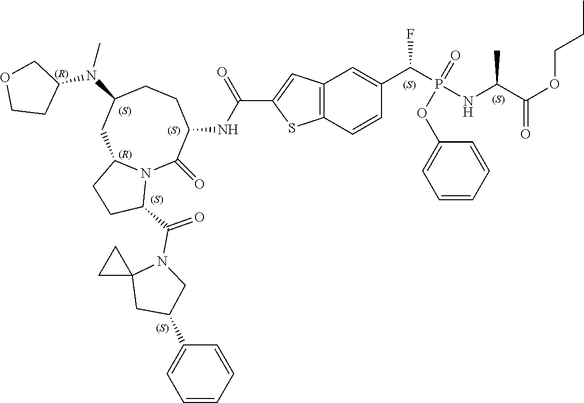 |
| C25 | propyl ((R)-((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 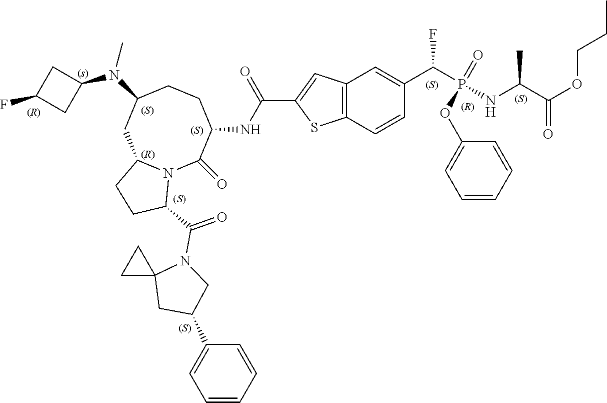 |
| C26 | propyl (((R)- fluoro(2- (((3S,6S,9S,10aR)- 9- (methyl(oxetan-3- ylmethyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 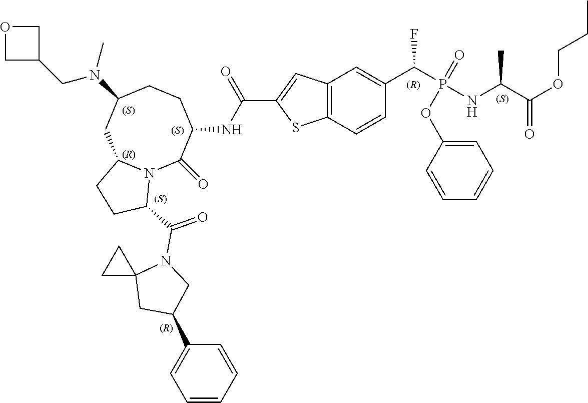 |
| C27 | propyl (((S)- fluoro(2- (((3S,6S,9S,10aR)- 9-(methyl((S)- tetrahydrofuran-3- yl)amino)-5-oxo- 3-((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 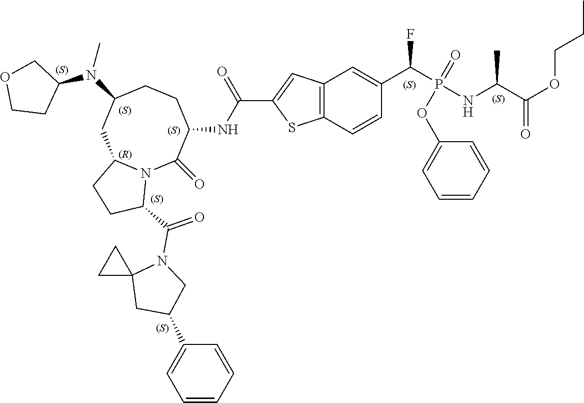 |
| C28 | propyl (((R)- fluoro(2- (((3S,6S,9S,10aR)- 9-(((1s,3R)-3- fluorocyclobutyl) (methyl)amino)-5- oxo-3-((R)-6- (pyridin-3-yl)-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 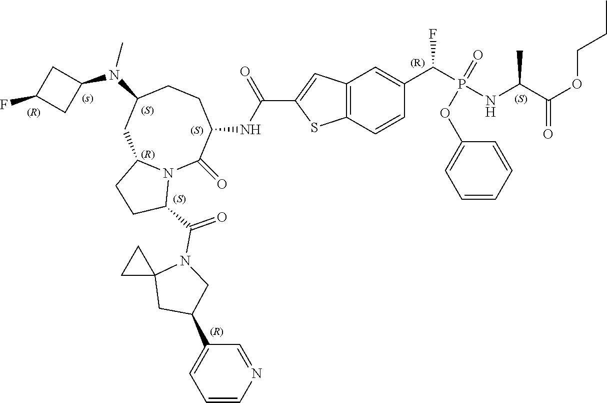 |
| C29 | propyl ((S)-((R)- fluoro(2- (((3S,6S,9S,10aR)- 9-morpholino-5- oxo-3-((R)-6- (pyridin-3-yl)-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 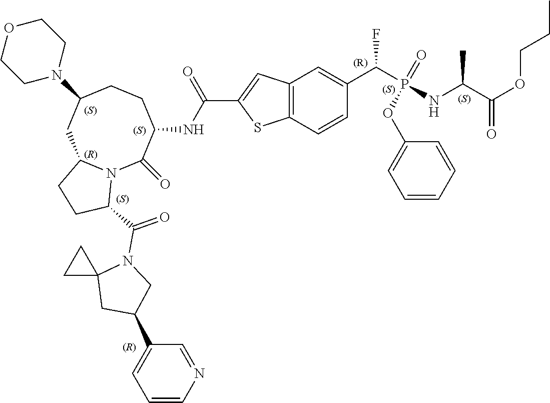 |
| C30 | propyl ((R)-((S)- fluoro(2- (((3S,6S,9S,10aR)- 9- (methyl(oxetan-3- yl)amino)-5-oxo- 3-((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 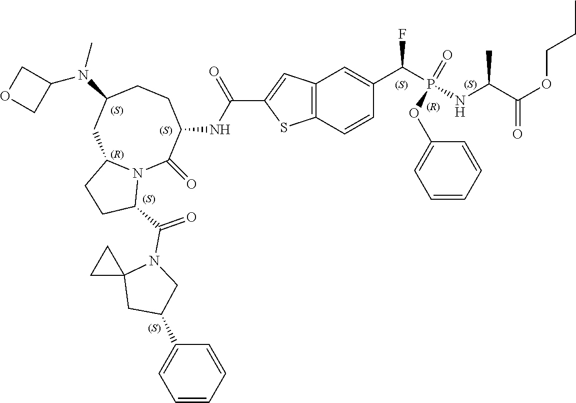 |
| C31 | propyl ((S)-((R)- (2- (((3S,6S,9S,10aR)- 3-((R)-6-(4- (dimethylamino) pyridin-3-yl)-4- azaspiro[2.4] heptane-4-carbonyl)- 9-morpholino-5- oxodecahydropyrrolo [1,2-a]azocin- 6- yl)carbamoyl) benzo [b]thiophen-5- yl)fluoromethyl) (phenoxy) phosphoryl)- L-alaninate | 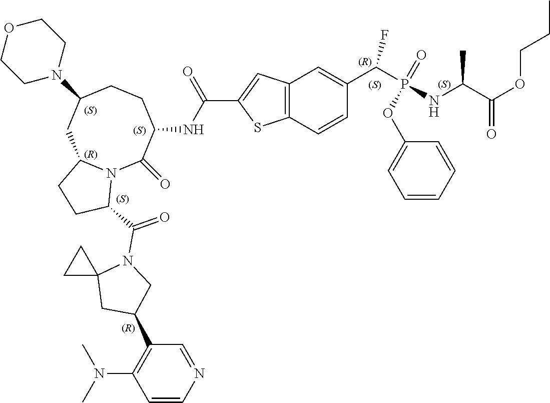 |
| C32 | propyl (((R)- fluoro(2- (((3S,6S,9S,10aR)- 9-(methyl((S)- tetrahydrofuran-3- yl)amino)-5-oxo- 3-((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 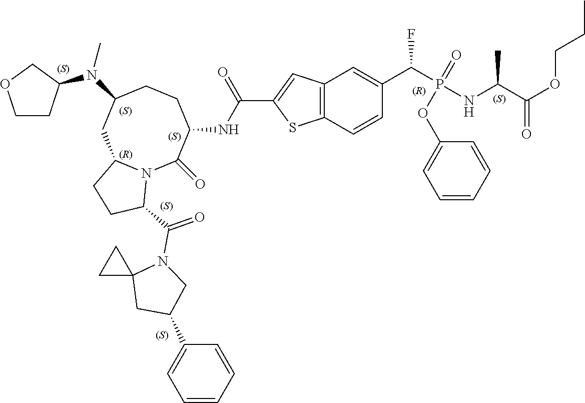 |
| C33 | propyl ((R)-((S)- fluoro(2- (((3S,6S,9S,10aR)- 9-morpholino-5- oxo-3-((R)-6- (pyridin-3-yl)-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 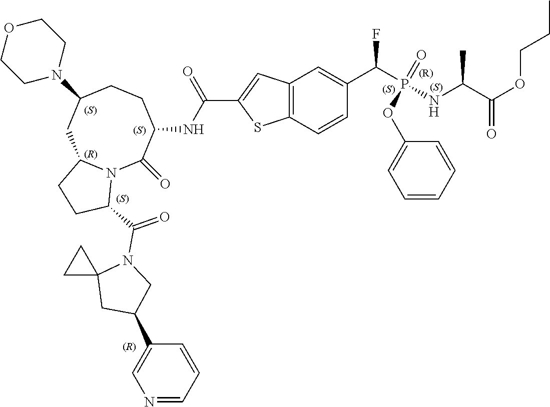 |
| C34 | propyl ((S)-((R)- fluoro(2- (((3S,6S,9S,10aR)- 9- (methyl(oxetan-3- yl)amino)-5-oxo- 3-((S)-6-phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 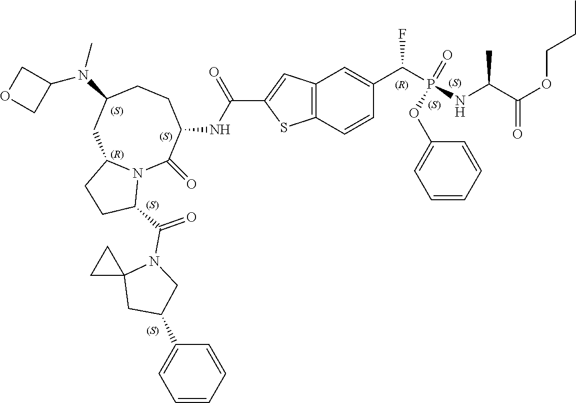 |
| C35 | propyl ((S)-((S)- fluoro(2- (((3S,6S,9S,10aR)- 9- (methyl(oxetan-3- yl)amino)-5-oxo- 3-((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 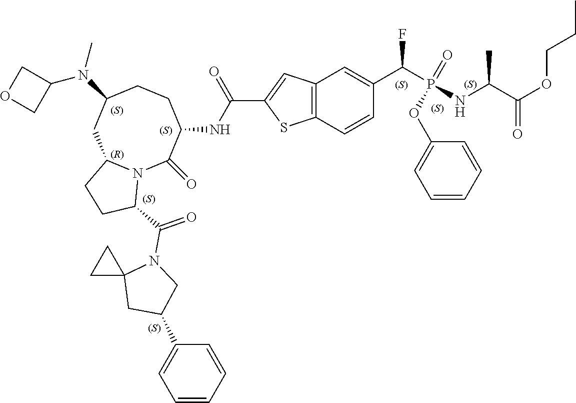 |
| C36 | propyl (((R)- fluoro(2- (((3S,6S,9S,10aR)- 3-((S)-6-(3- fluorophenyl)-4- azaspiro [2.4] heptane-4-carbonyl)-9- (methyl(oxetan-3- yl)amino)-5- oxodecahydropyrrolo [1,2-a]azocin- 6- yl)carbamoyl) benzo [b]thiophen-5- yl)methyl) (phenoxy) phosphoryl)-L- alaninate | 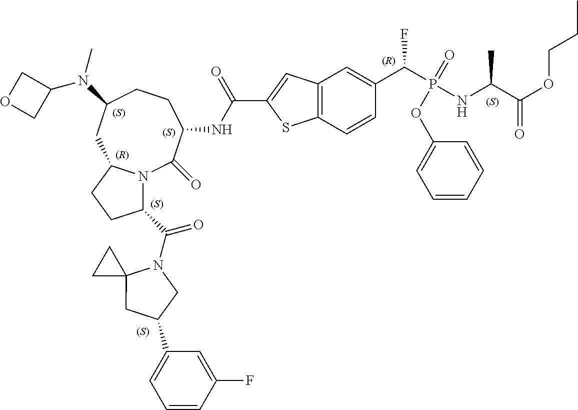 |
| C37 | propyl ((R)-((R)- fluoro(2- (((3S,6S,9S,10aR)- 9- (methyl(oxetan-3- yl)amino)-5-oxo- 3-((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 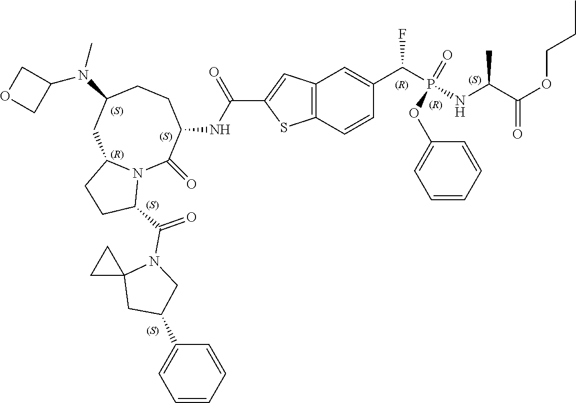 |
| C38 | propyl (((R)-(2- (((3S,6S,9S,10aR)- 3-((6S,7R)-7- cyano-6-phenyl-4- azaspiro [2.4] heptane-4-carbonyl)- 9-morpholino-5- oxodecahydropyrrolo [1,2-a]azocin- 6- yl)carbamoyl) benzo [b]thiophen-5- yl)fluoromethyl) (phenoxy) phosphoryl)- L-alaninate | 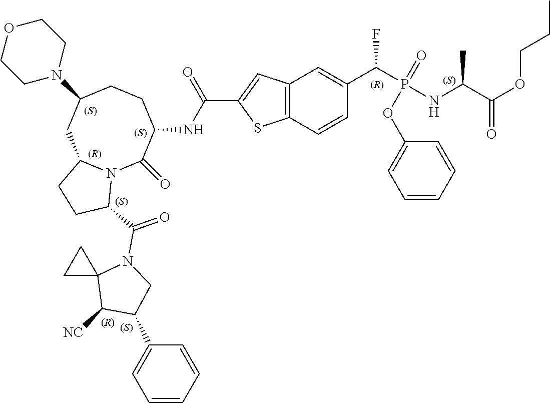 |
| C39 | propyl (((R)- fluoro(2- (((3S,6S,9S,10aR)- 9-morpholino-5- oxo-3-((R)-6- (pyridin-3-yl)-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 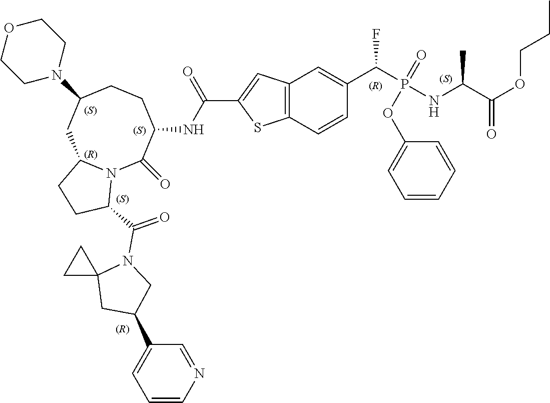 |
| C40 | propyl ((R)-((R)- fluoro(2- (((3S,6S,9S,10aR)- 9-morpholino-5- oxo-3-((R)-6- (pyridin-3-yl)-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 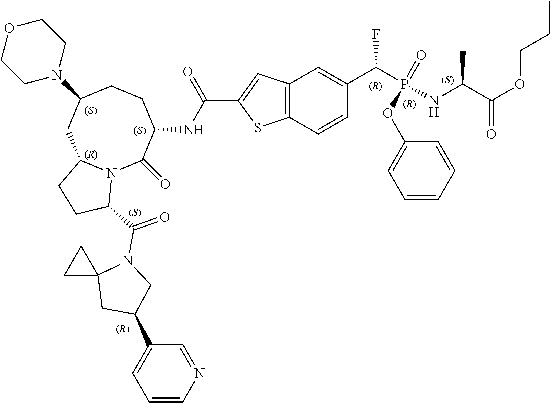 |
| C41 | propyl ((R)-((R)- fluoro(2- (((3S,6S,9S,10aR)- 9-morpholino-5- oxo-3-((S)-6- (pyridin-3-yl)-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 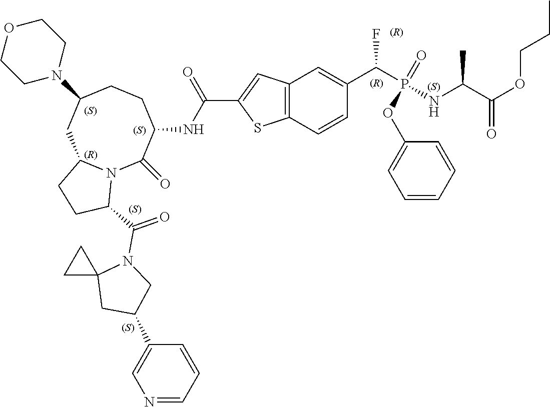 |
| C42 | propyl ((S)-((R)- fluoro(2- (((3S,6S,9S,10aR)- 9-morpholino-5- oxo-3-((S)-6- (pyridin-3-yl)-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 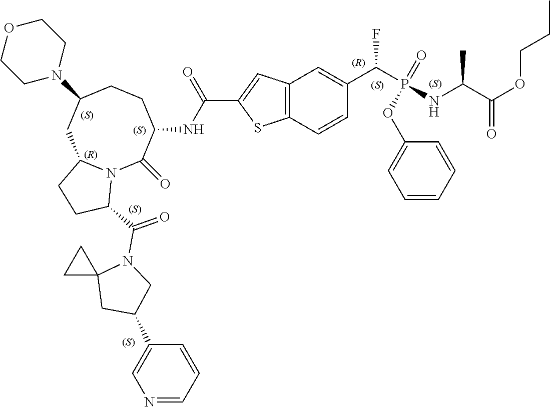 |
| C43 | propyl ((S)-((S)- fluoro(2- (((3S,6S,9S,10aR)- 9-morpholino-5- oxo-3-((R)-6- (pyridin-3-yl)-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 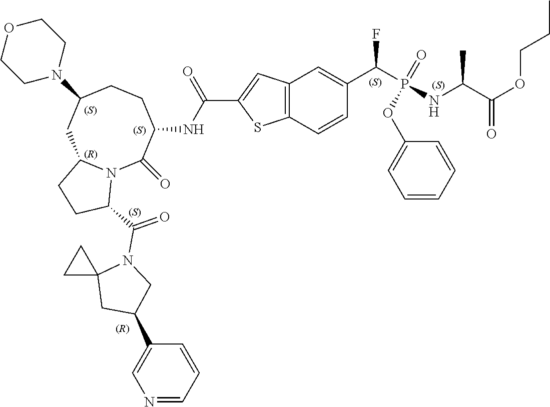 |
| C44 | propyl ((R)-((R)- (2- (((3S,6S,9S,10aR)- 3-((6S,7R)-7- cyano-6-(pyridin- 3-y1)-4- azaspiro[2.4] heptane-4-carbonyl)- 9-morpholino-5- oxodecahydropyrrolo [1,2-a]azocin- 6- yl)carbamoyl) benzo [b]thiophen-5- yl)fluoromethyl) (phenoxy) phosphoryl)- L-alaninate | 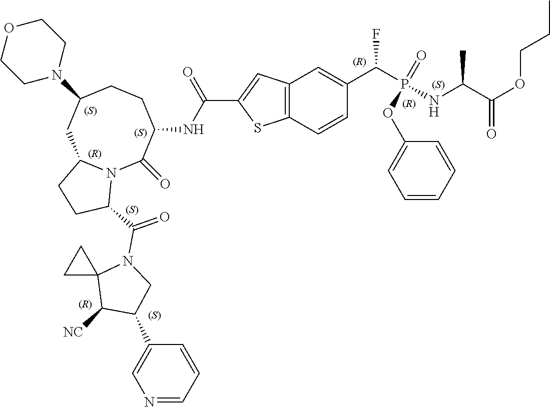 |
| C45 | propyl ((S)-((R)- (2- (((3S,6S,9S,10aR)- 3-((6S,7R)-7- cyano-6-(pyridin- 3-yl)-4- azaspiro[2.4] heptane-4-carbonyl)- 9-morpholino-5- oxodecahydropyrrolo [1,2-a]azocin- 6- yl)carbamoyl) benzo [b]thiophen-5- yl)fluoromethyl) (phenoxy) phosphoryl)- L-alaninate | 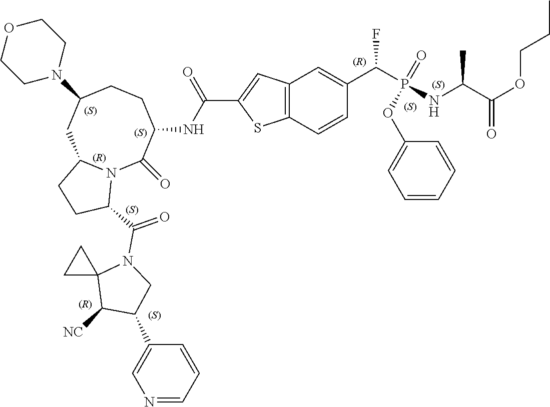 |
| C46 | propyl ((R)-((R)- (2- (((3S,6S,9S,10aR)- 3-((R)-6-(4- (dimethylamino) pyridin-3-yl)-4- azaspiro[2.4] heptane-4-carbonyl)- 9-morpholino-5- oxodecahydropyrrolo [1,2-a]azocin- 6- yl)carbamoyl) benzo [b]thiophen-5- yl)fluoromethyl) (phenoxy) phosphoryl)- L-alaninate | 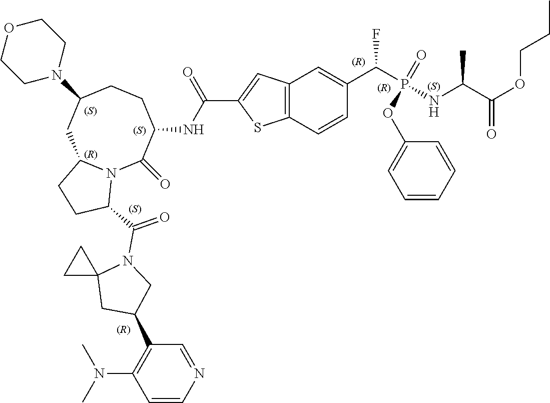 |
| C47 | propyl ((R)-((S)- (2- (((3S,6S,9S,10aR)- 3-((R)-6-(4- (dimethylamino) pyridin-3-yl)-4- azaspiro[2.4] heptane-4-carbonyl)- 9-morpholino-5- oxodecahydropyrrolo [1,2-a]azocin- 6- yl)carbamoyl) benzo [b]thiophen-5- yl)fluoromethyl) (phenoxy) phosphoryl)- L-alaninate | 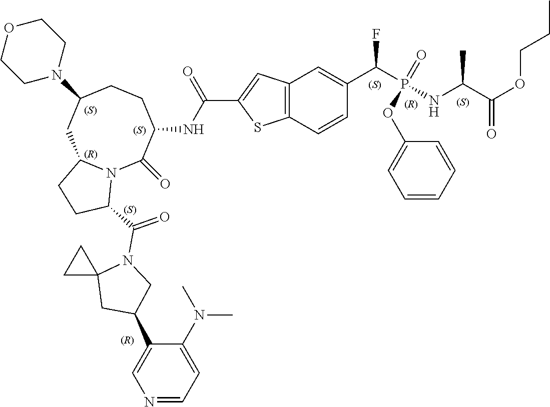 |
| C48 | propyl ((S)-((S)- (2- (((3S,6S,9S,10aR)- 3-((R)-6-(4- (dimethylamino) pyridin-3-yl)-4- azaspiro[2.4] heptane-4-carbonyl)- 9-morpholino-5- oxodecahydropyrrolo [1,2-a]azocin- 6- yl)carbamoyl) benzo [b]thiophen-5- yl)fluoromethyl) (phenoxy) phosphoryl)- L-alaninate | 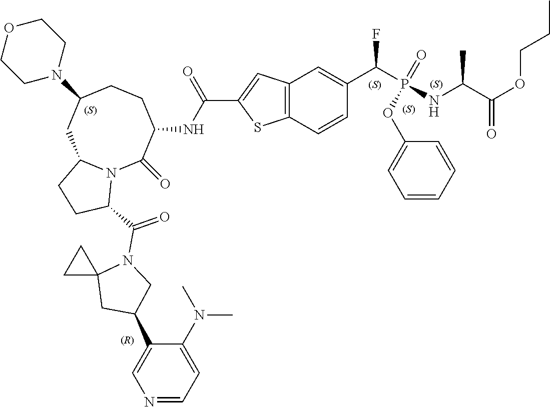 |
| C49 | isopropyl ((S)- ((R)-fluoro(2- (((3S,6S,9S,10aR)- 9-morpholino-5- oxo-3-((R)-6- (pyridin-3-yl)-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 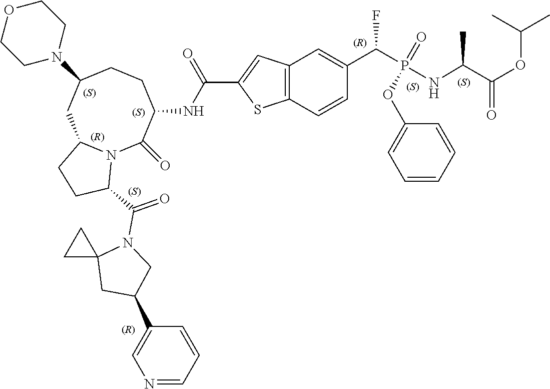 Phosphorus absolute stereochemistry arbitrarily assigned |
| C50 | isopropyl ((R)- ((R)-fluoro(2- (((3S,6S,9S,10aR)- 9-morpholino-5- oxo-3-((R)-6- (pyridin-3-yl)-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 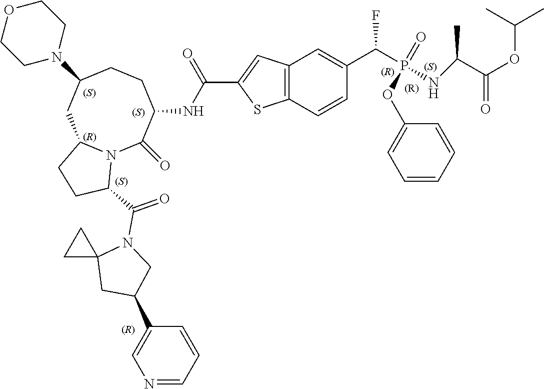 Phosphorus absolute stereochemistry arbitrarily assigned |
| C51 | propyl (((S)- fluoro(2- (((3S,6S,9S,10aR)- 9-(methyl(((R)- oxetan-2- yl)methyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 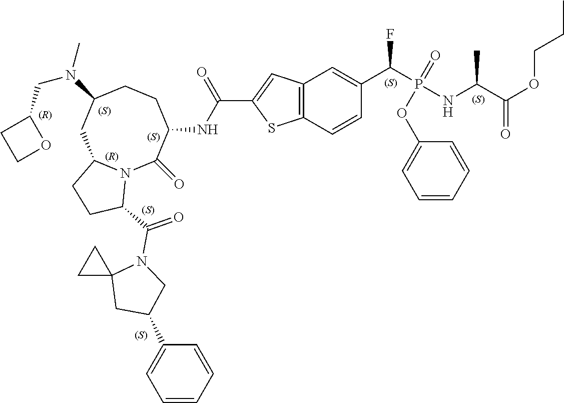 |
| C52 | benzyl (((R)-(2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 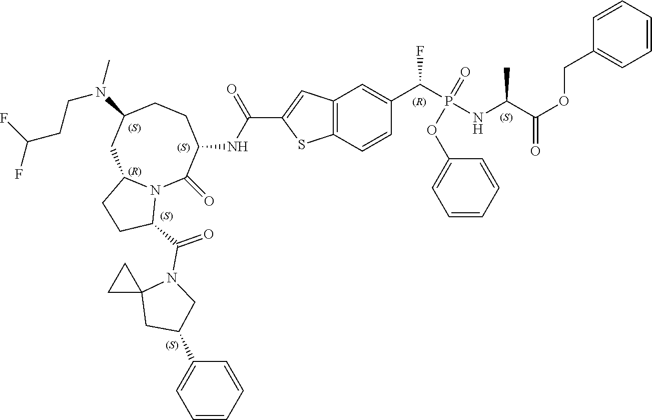 |
| C53 | propyl (2S)-2- ((((R)-(2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl) amino) butanoate. | 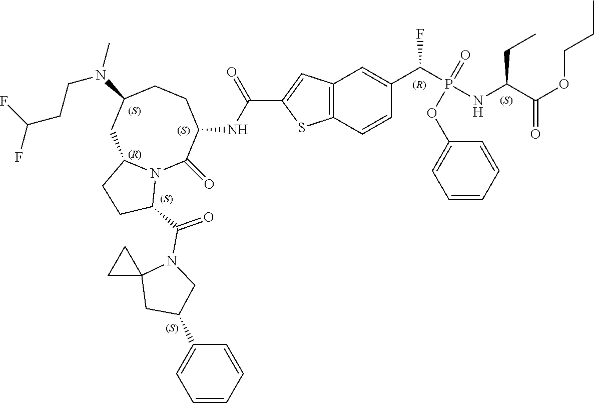 |
| C54 | 2-methoxyethyl (((R)-(2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 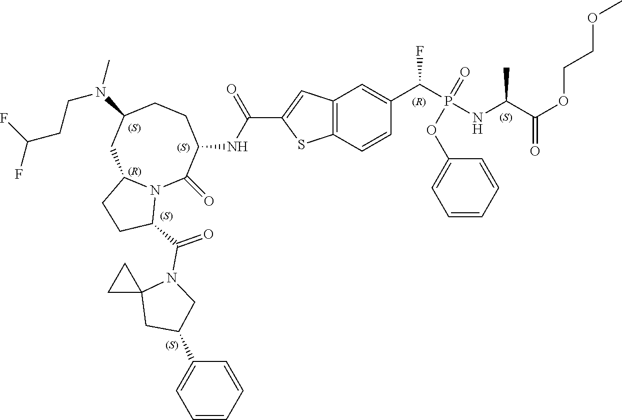 |
| C55 | propyl (((R)-(2- (((3S,6S,9S,10aR)- 9-(ethyl((1s,3R)- 3- fluorocyclobutyl)a mino)-5-oxo-3- ((S)-6-phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 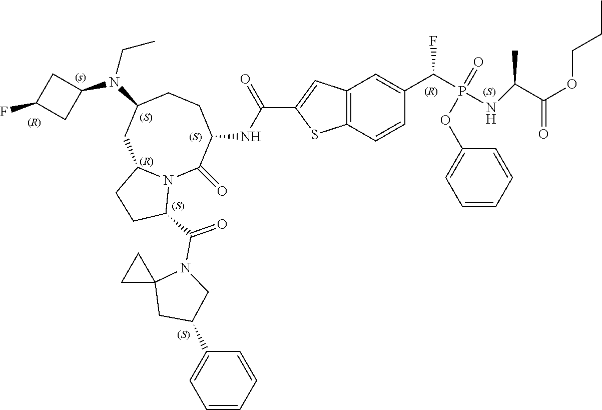 |
| C56 | propyl (((S)-(2- (((3S,6S,9S,10aR)- 9-(ethyl((1s,3R)- 3- fluorocyclobutyl)a mino)-5-oxo-3- ((S)-6-phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 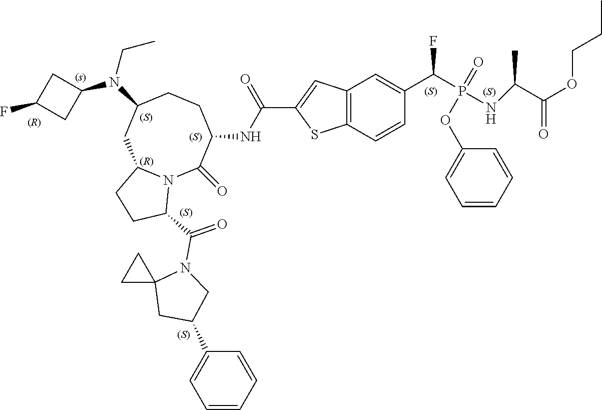 |
| C57 | isopropyl (((R)- (2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 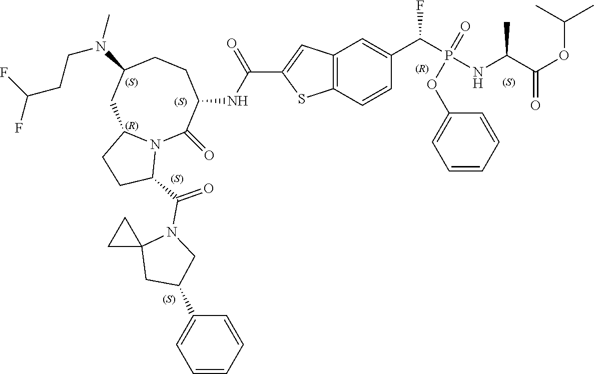 |
| C58 | ethyl (((R)-(2- (((3S,6S,9S,10aR)- 9-((3,3- difluoropropyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 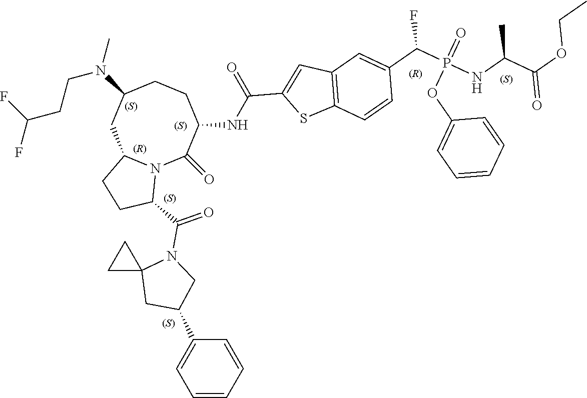 |
| C59 | propyl ((R)-((R)- fluoro(2- (((3S,6S,9S,10aR)- 9-(methyl((R)- tetrahydrofuran-3- yl)amino)-5-oxo- 3-((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 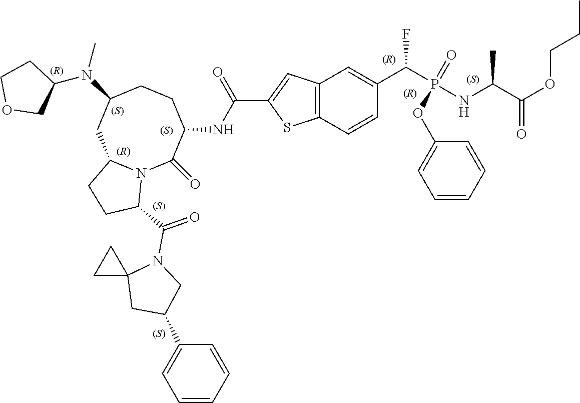 |
| C60 | propyl ((S)-((R)- fluoro(2- (((3S,6S,9S,10aR)- 9-(methyl((R)- tetrahydrofuran-3- yl)amino)-5-oxo- 3-((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 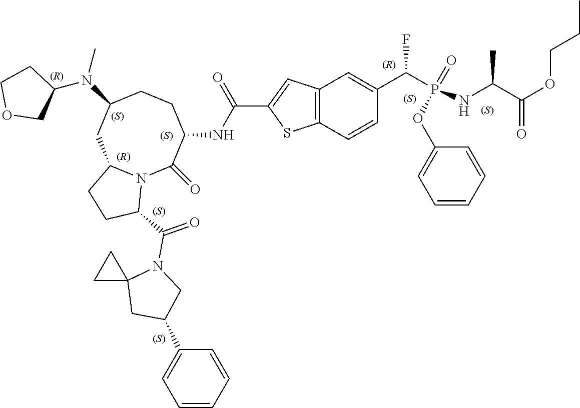 |
| C61 | cyclobutyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 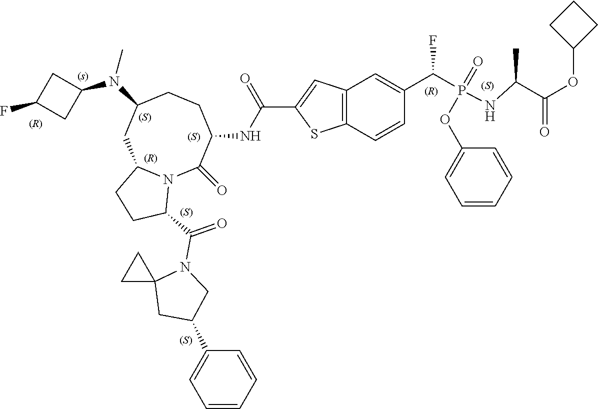 |
| C62 | cyclobutyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 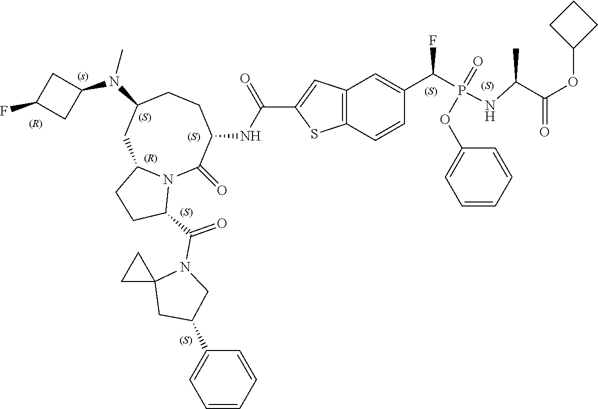 |
| C63 | propyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 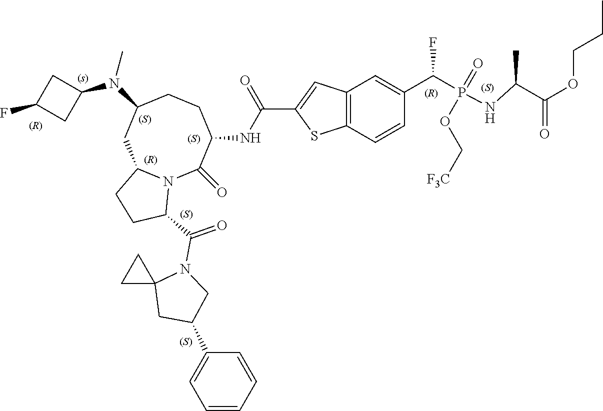 |
| C64 | propyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 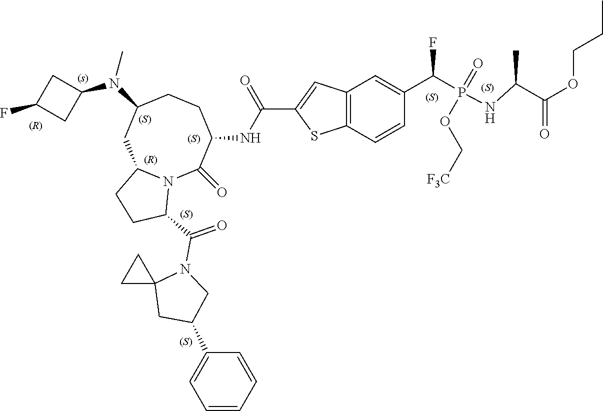 |
| C65 | propyl (((R)-(2- (((3S,6S,9S,10aR)- 9-(((1s,3R)-3- cyanocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 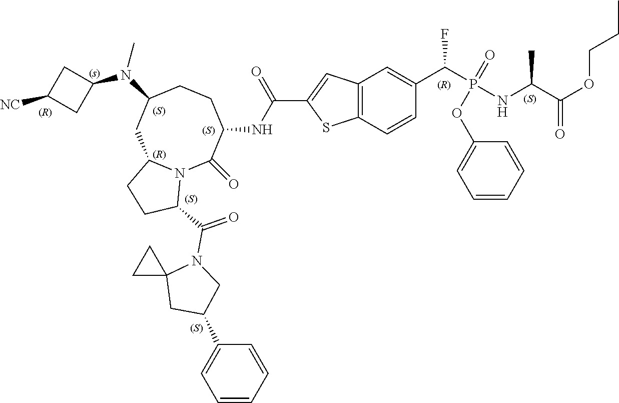 |
| C66 | (3,3- difluorocyclobutyl) methyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 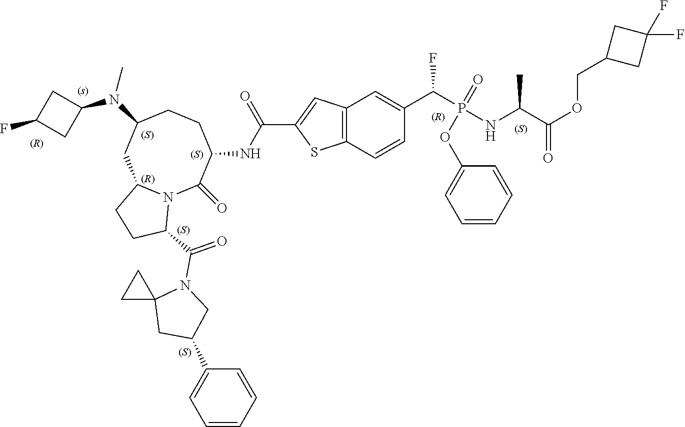 |
| C67 | (3,3- difluorocyclobutyl) methyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 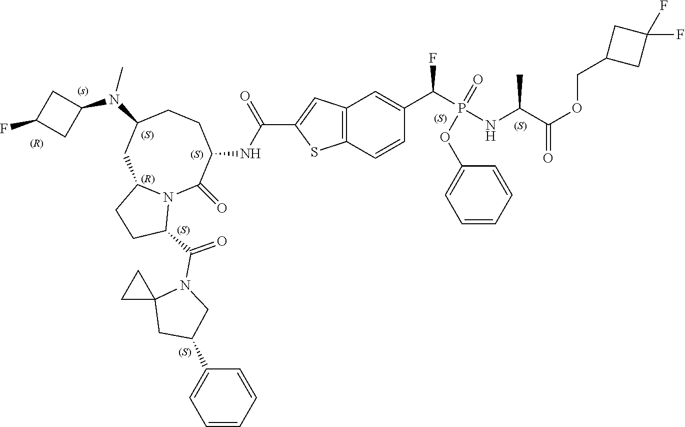 |
| C68 | isopropyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 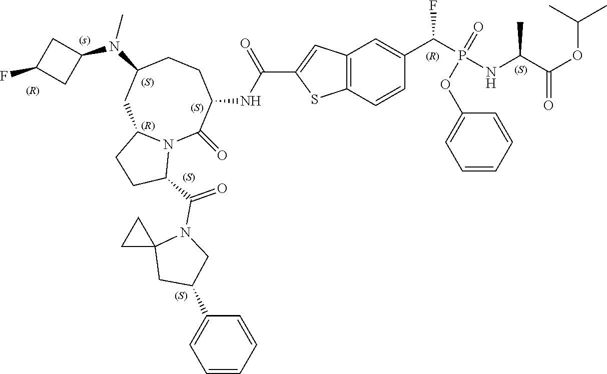 |
| C69 | isopropyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 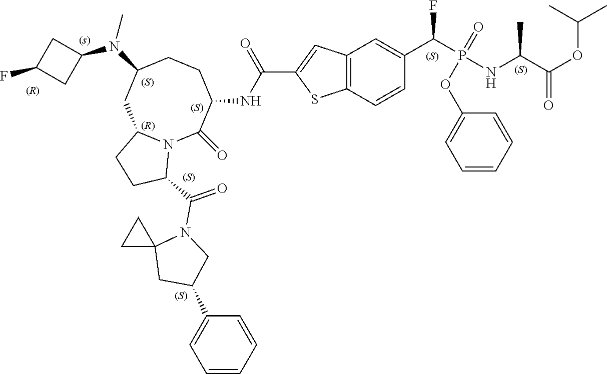 |
| C70 | propyl ((R)-((R)- (2- (((3S,6S,9S,10aR)- 9-(((1s,3R)-3- (difluoromethyl) cyclobutyl)(methyl) amino)-5-oxo-3- ((S)-6-phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 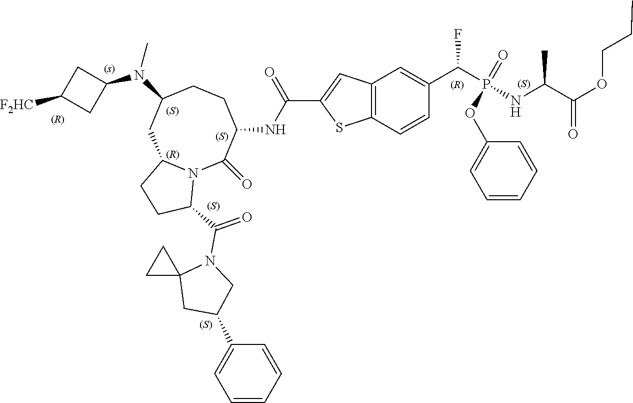 |
| C71 | propyl ((S)-((R)- (2- (((3S,6S,9S,10aR)- 9-(((1s,3R)-3- (difluoromethyl) cyclobutyl)(methyl) amino)-5-oxo-3- ((S)-6-phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 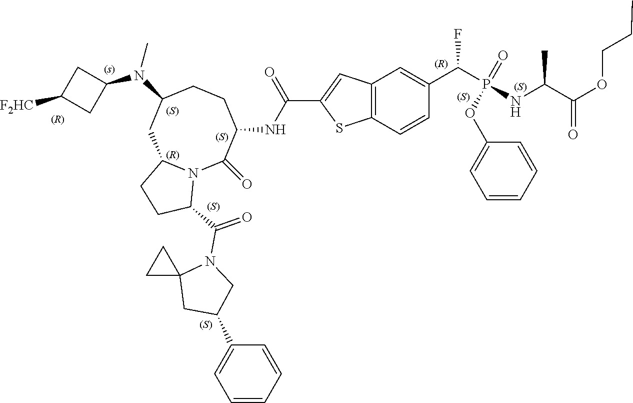 |
| C72 | (1r,3S)-3- fluorocyclobutyl (((S)-fluoro(2- (((3S,6S,9S,10aR)- 9-(((1s,3R)-3- fluorocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 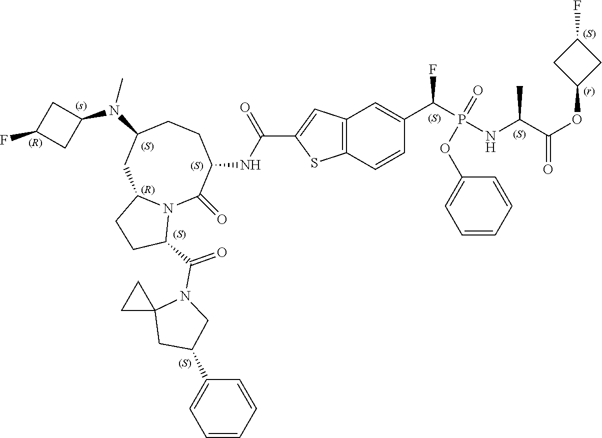 |
| C73 | (1r,3S)-3- fluorocyclobutyl (((R)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 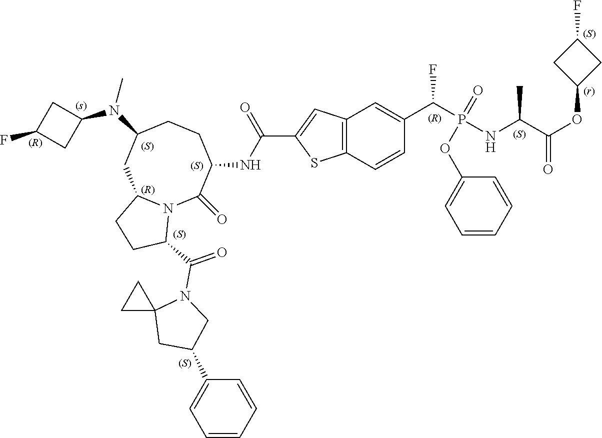 |
| C74 | 2-ethylbutyl (((R)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 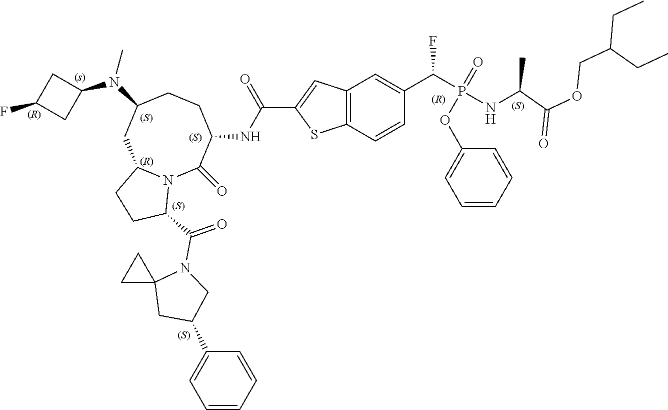 |
| C75 | neopentyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 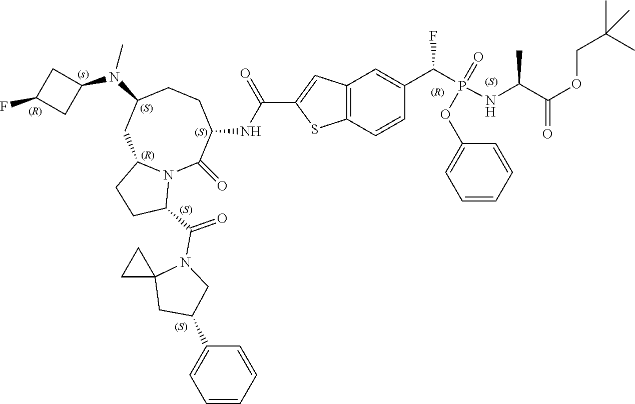 |
| C76 | (1s,3R)-3- fluorocyclobutyl (((S)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 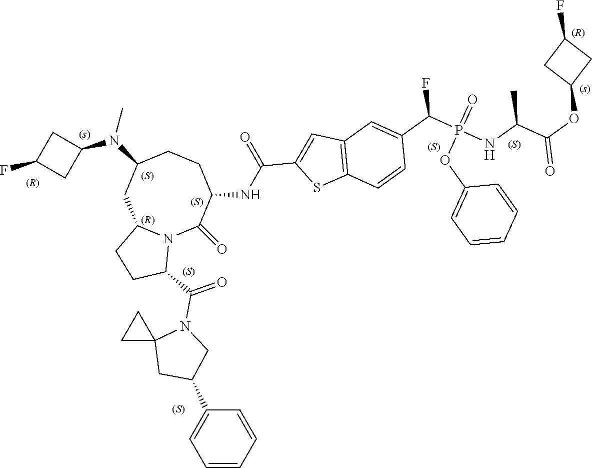 |
| C77 | (1s,3R)-3- fluorocyclobutyl (((R)-fluoro(2- (((3S,6S,9S,10aR)- 9-(((1s,3R)-3- fluorocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 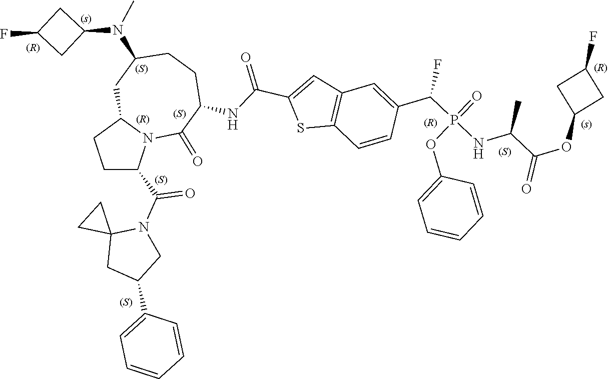 |
| C78 | isopropyl ((R)- ethoxy((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 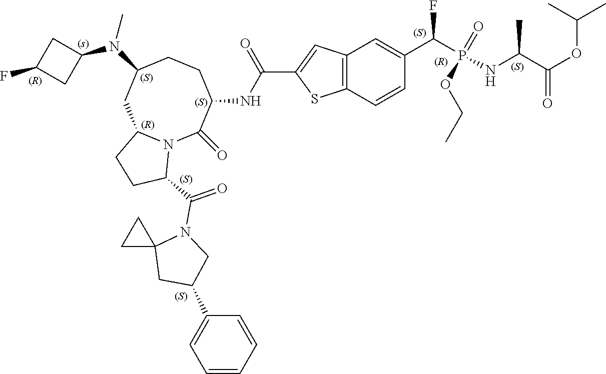 Phosphorus absolute stereochemistry arbitrarily assigned |
| C79 | 2-ethylbutyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 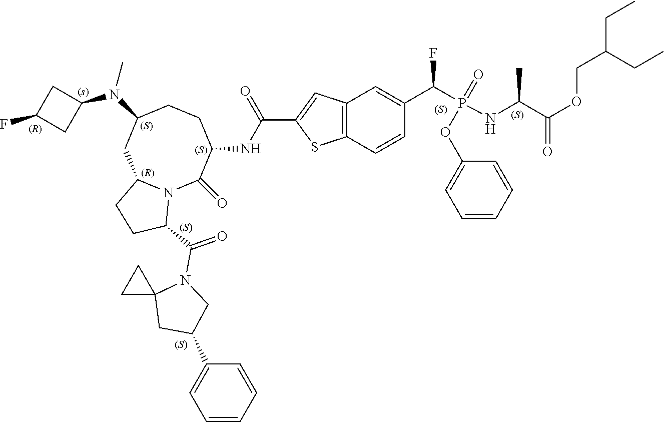 |
| C80 | neopentyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 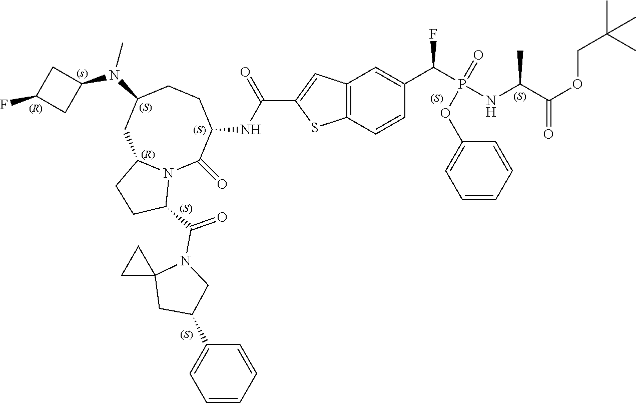 |
| C81 | propyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)- D-alaninate | 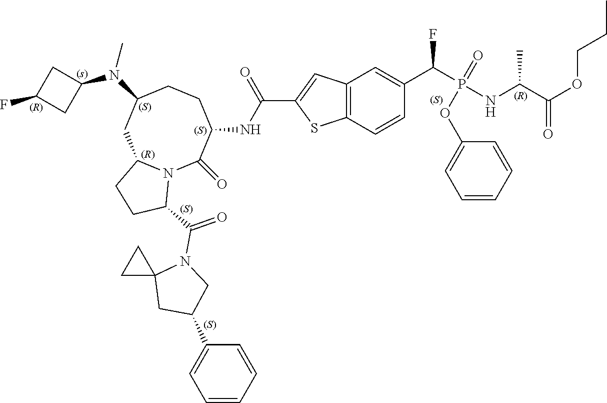 |
| C82 | propyl ((R)-((R)- fluoro(2- (((3S,6S,9S,10aR)- 9-(methyl(4,4,4- trifluorobutyl) amino)-5-oxo-3-((S)- 6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 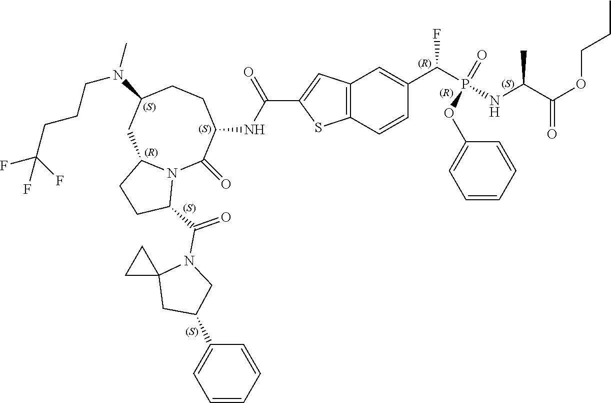 |
| C83 | isopropyl ((S)- ethoxy((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 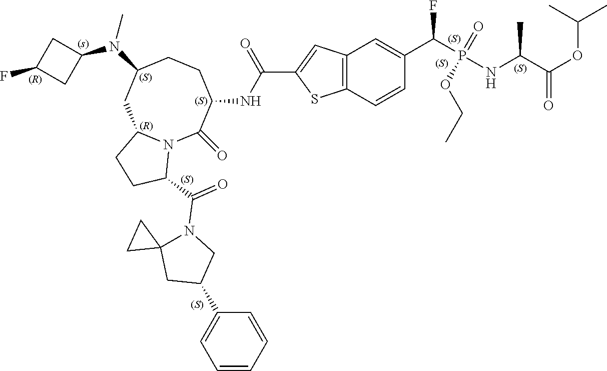 Phosphorus absolute stereochemistry arbitrarily assigned |
| C84 | propyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)- D-alaninate | 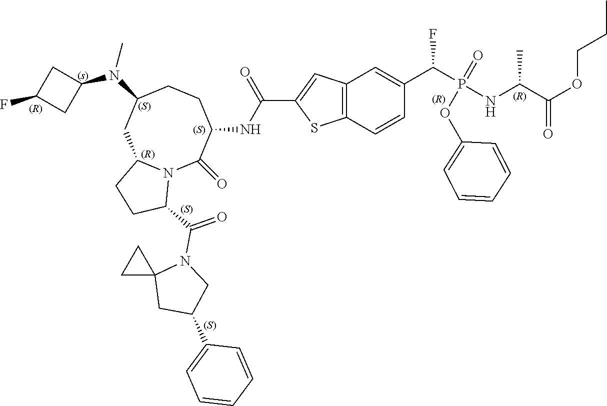 |
| C85 | propyl (((R)- fluoro(2- (((3S,6S,9S,10aR)- 9-(((1s,3R)-3- fluorocyclobutyl) (methyl)amino)-5- oxo-3-(4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 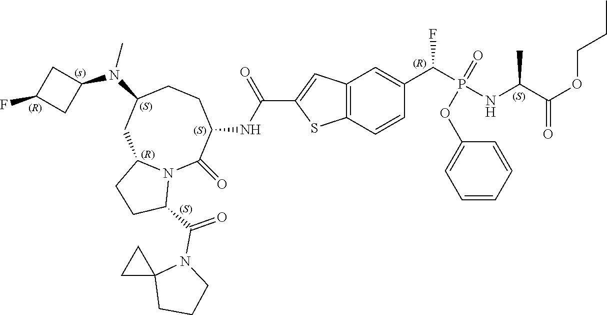 |
| C86 | isopropyl ((R)- ethoxy((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 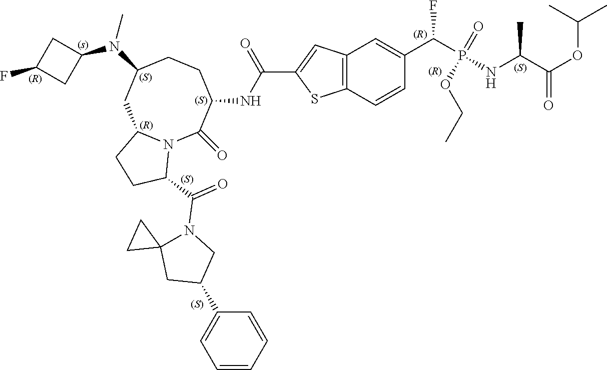 Phosphorus absolute stereochemistry arbitrarily assigned |
| C87 | propyl (((S)- fluoro(2- (((3S,6S,9S,10aR)- 9-(methyl(4,4,4- trifluorobutyl) amino)-5-oxo-3-((S)- 6-phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 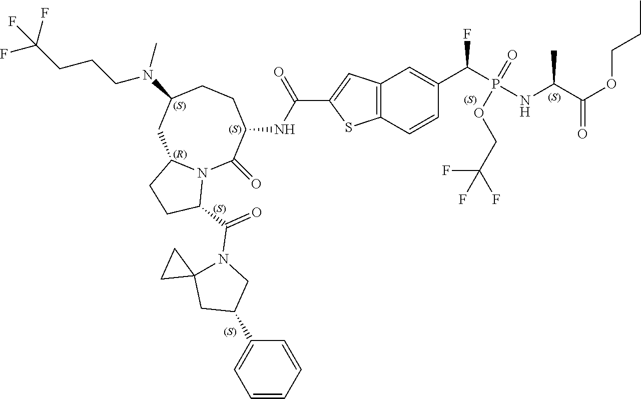 |
| C88 | propyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(propox y)phosphoryl)-L- alaninate | 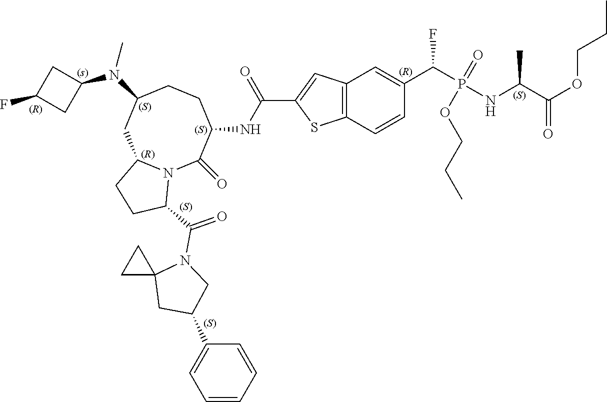 |
| C89 | cyclobutyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 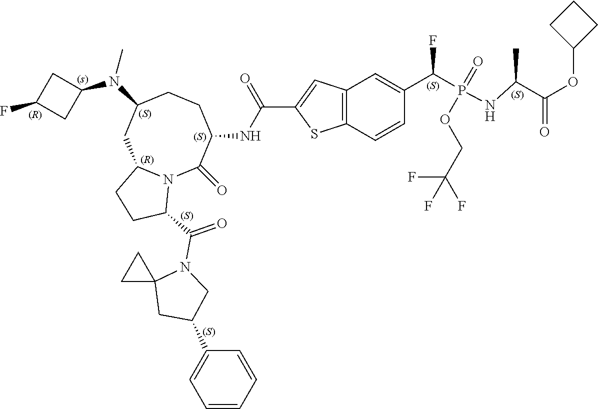 |
| C90 | propyl (((R)-(2- (((3S,6S,9S,10aR)- 9-(((S)-3,3- difluorocyclopent yl)(methyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (2,2,2- trifluoroethoxy) phosphoryl)-L- alaninate | 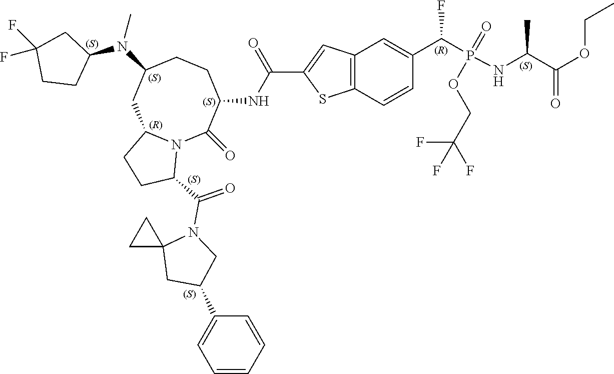 |
| C91 | propyl (((R)- fluoro(2- (((3S,6S,9S,10aR)- 9-(methyl(4,4,4- trifluorobutyl) amino)-5-oxo-3-((S)- 6-phenyl-4- azaspiro [2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 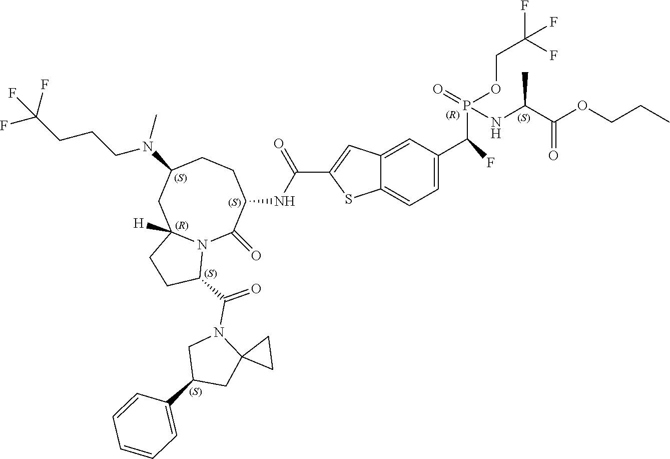 |
| C92 | propyl (cyclopropoxy((R)- fluoro(2- (((3S,6S,9S,10aR)- 9-(((1s,3R)-3- fluorocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) phosphoryl)- L-alaninate | 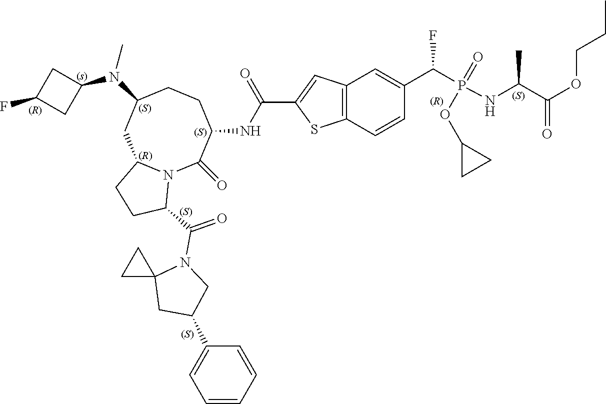 |
| C93 | isopropyl ((S)- ethoxy((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 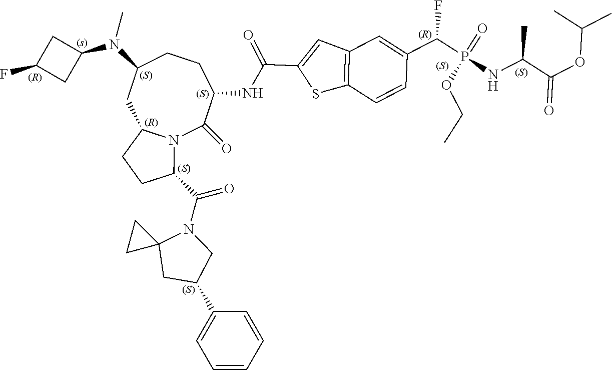 Phosphorus absolute stereochemistry arbitrarily assigned |
| C94 | isopropyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 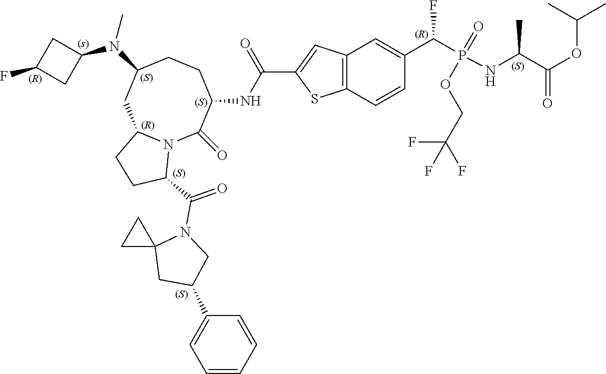 |
| C95 | propyl (((S)-(2- (((3S,6S,9S,10aR)- 9-(((S)-3,3- difluorocyclopent yl)(methyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (2,2,2- trifluoroethoxy) phosphoryl)-L- alaninate | 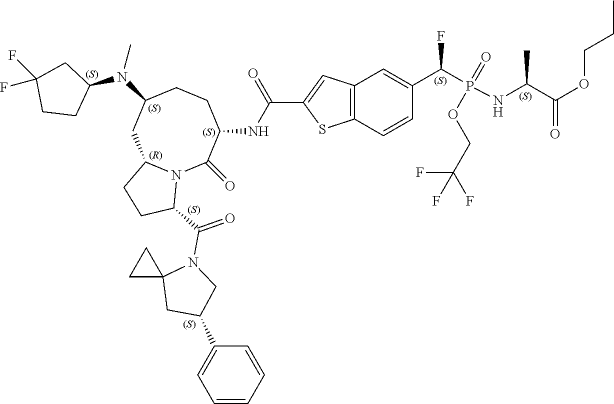 |
| C96 | propyl (((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 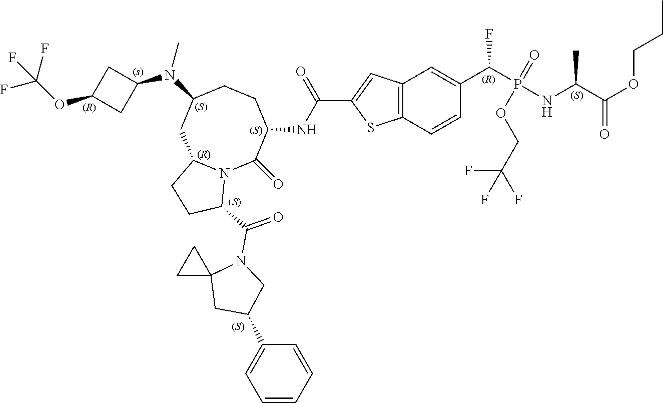 |
| C97 | propyl (((S)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 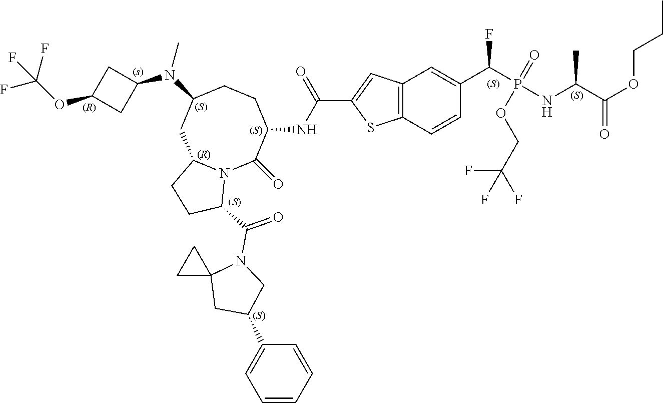 |
| C98 | propyl (((R)-(2- (((3S,6S,9S,10aR)- 9-(ethyl((3- methyloxetan-3- yl)methyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 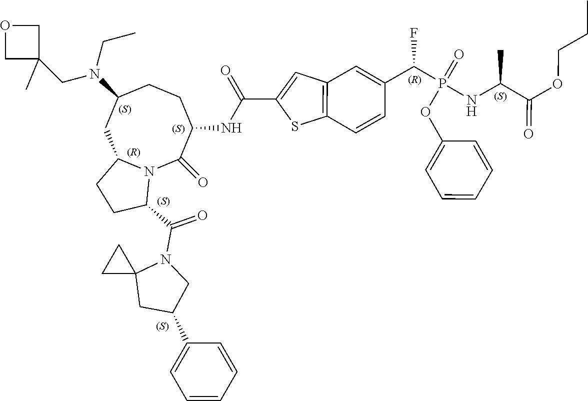 |
| C99 | propyl (((R)-(2- (((3S,6S,9S,10aR)- 9-(ethyl((1s,3R)- 3- (trifluoromethoxy) cyclobutyl)amino)- 5-oxo-3-((S)-6- phenyl-4- azaspiro [2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 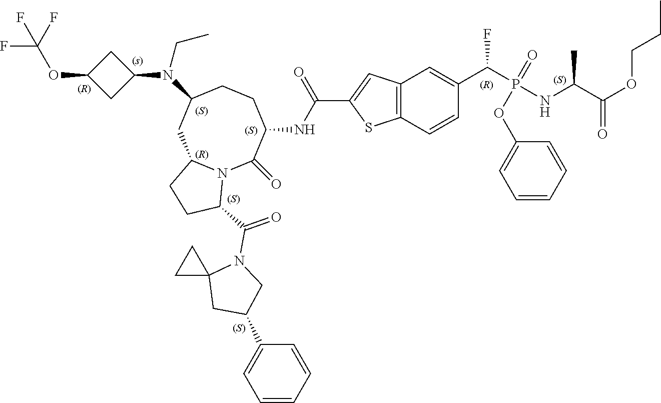 |
| C100 | propyl (((R)-(2- (((3S,6S,9S,10aR)- 9-(ethyl(4,4,4- trifluorobutyl) amino)-5-oxo-3-((S)- 6-phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 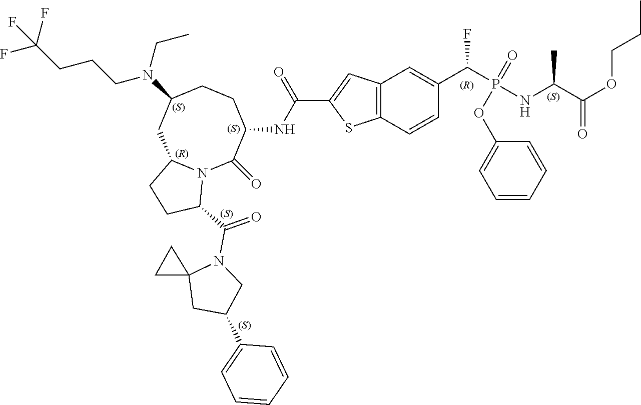 |
| C101 | propyl (((S)-(2- (((3S,6S,9S,10aR)- 9-(ethyl(4,4,4- trifluorobutyl) amino)-5-oxo-3-((S)- 6-phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 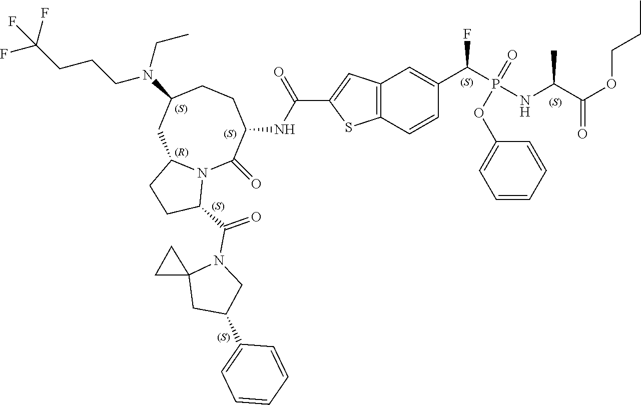 |
| C102 | cyclobutyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 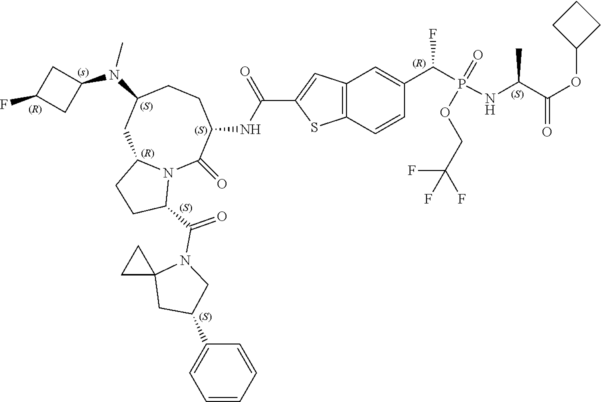 |
| C103 | isopropyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 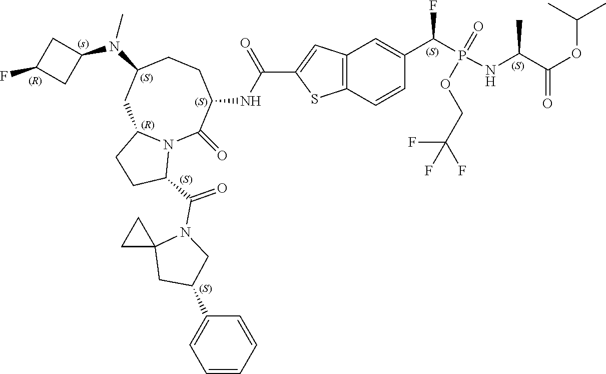 |
| C104 | isobutyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 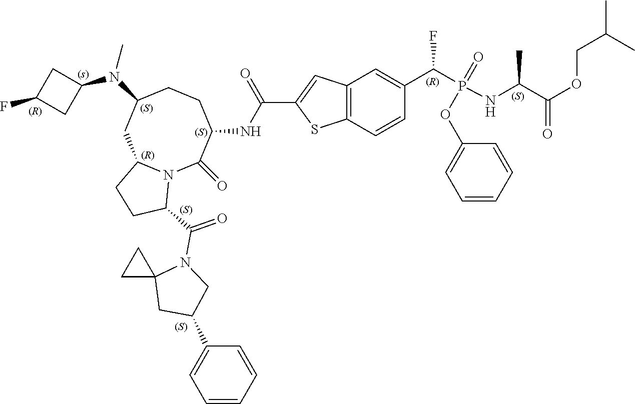 |
| C105 | isobutyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 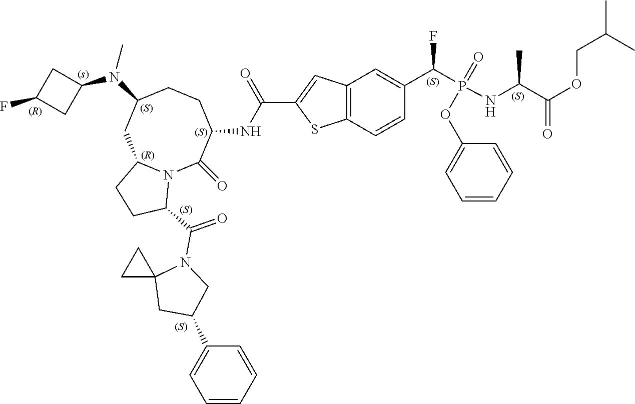 |
| C106 | cyclobutyl (ethoxy((R)- fluoro(2- (((3S,6S,9S,10aR)- 9-(((1s,3R)-3- fluorocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) phosphoryl)- L-alaninate | 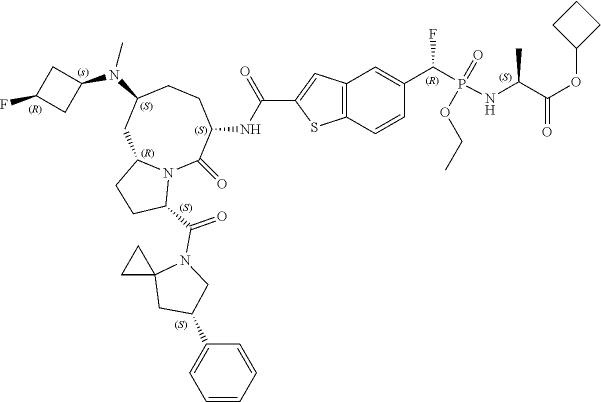 |
| C107 | cyclobutyl (ethoxy((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 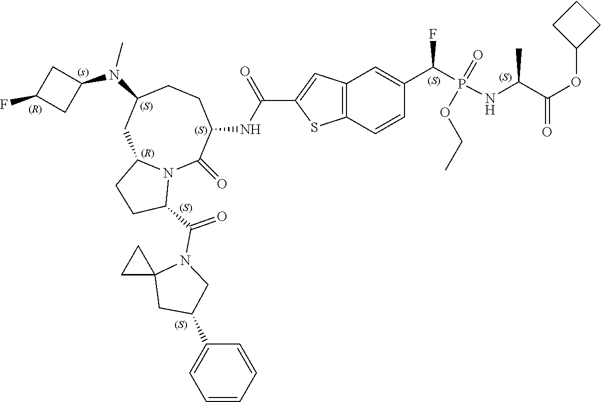 |
| C108 | propyl (((R)-(2- (((3S,6S,9S,10aR)- 9-(ethyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 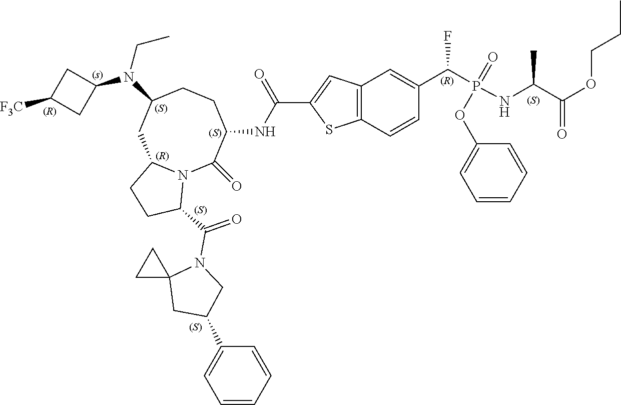 |
| C109 | propyl (((S)-(2- (((3S,6S,9S,10aR)- 9-(ethyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 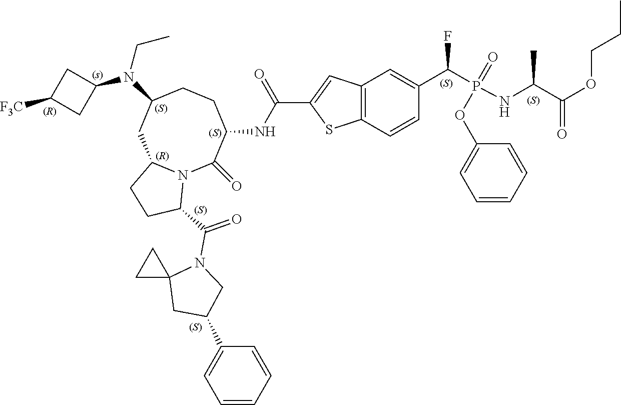 |
| C110 | propyl (((R)-(2- (((3S,6S,9S,10aR)- 9-(((1s,3R)-3- cyanocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (2,2,2- trifluoroethoxy) phosphoryl)-L- alaninate | 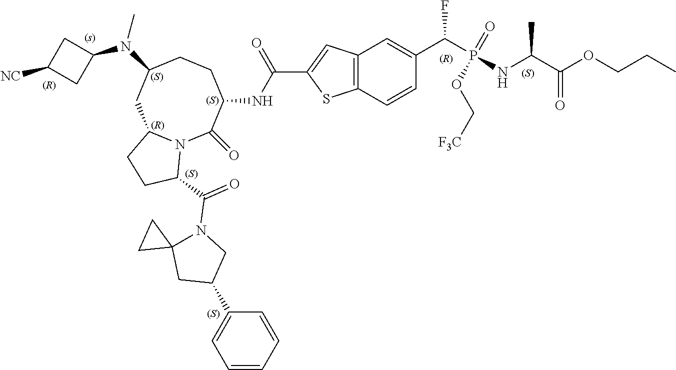 |
| C111 | cyclobutyl (((R)- (2- (((3S,6S,9S,10aR)- 9-(((1s,3R)-3- cyanocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 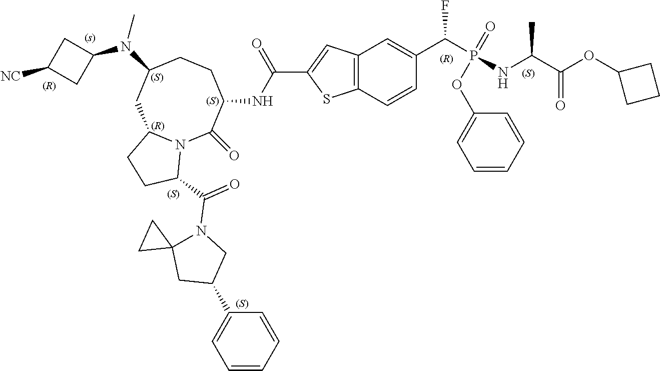 |
| C112 | cyclobutyl (((S)- (2- (((3S,6S,9S,10aR)- 9-(((1s,3R)-3- cyanocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 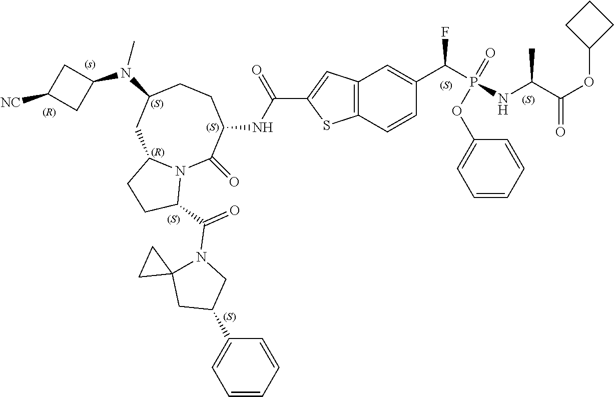 |
| C113 | cyclobutyl (((R)- fluoro(2- (((3S,6S,9S,10aR)- 9- (methyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 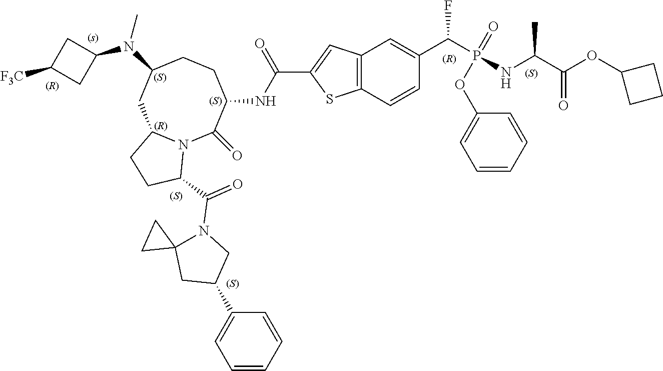 |
| C114 | propyl (((R)- fluoro(2- (((3S,6S,9S,10aR)- 9- (methyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 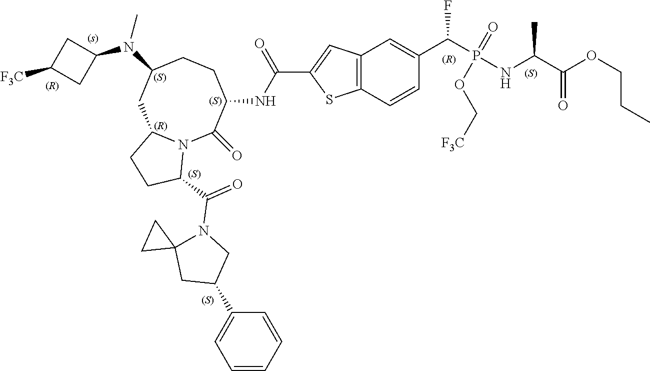 |
| C115 | propyl ((S)-((S)- fluoro(2- (((3S,6S,9S,10aR)- 9-(methyl(4,4,4- trifluorobutyl) amino)-5-oxo-3-((S)- 6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 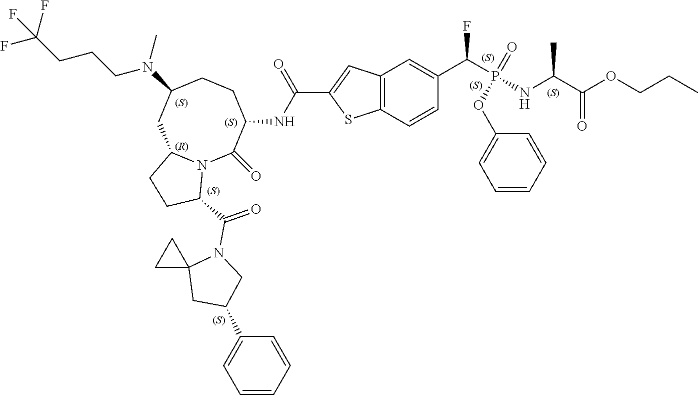 |
| C116 | propyl (cyclopropoxy((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 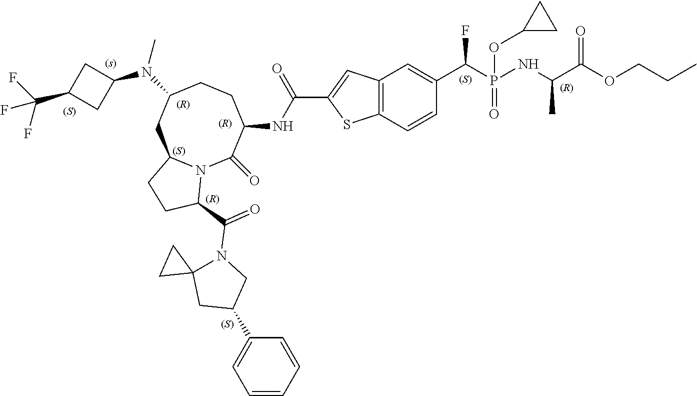 |
| C117 | propyl (cyclopropoxy((R)- fluoro(2- (((3S,6S,9S,10aR)- 9- (methyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 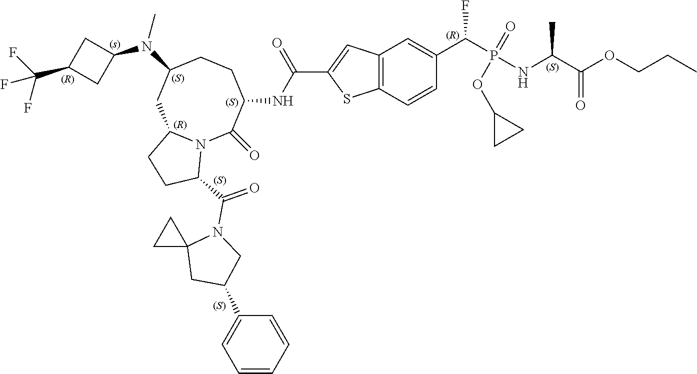 |
| C118 | isopropyl (((S)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 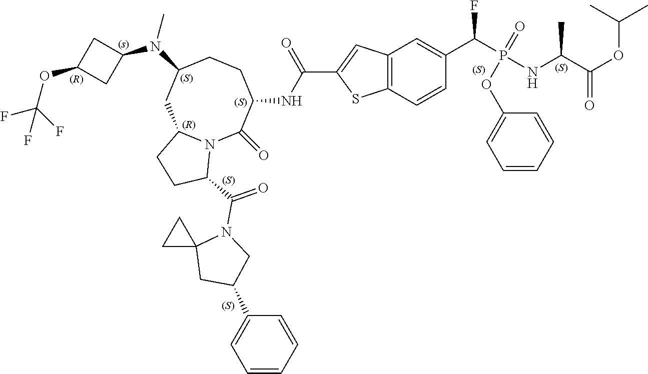 |
| C119 | propyl (((S)-(2- (((3S,6S,9S,10aR)- 9-(ethyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 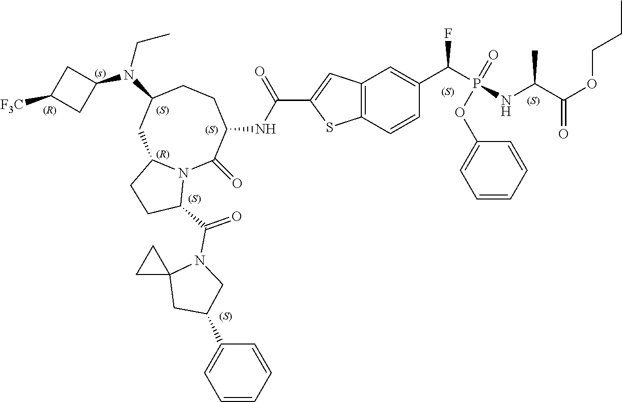 |
| C120 | propyl ((R)-((S)- fluoro(2- (((3S,6S,9S,10aR)- 9-(methyl(4,4,4- trifluorobutyl) amino)-5-oxo-3-((S)- 6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 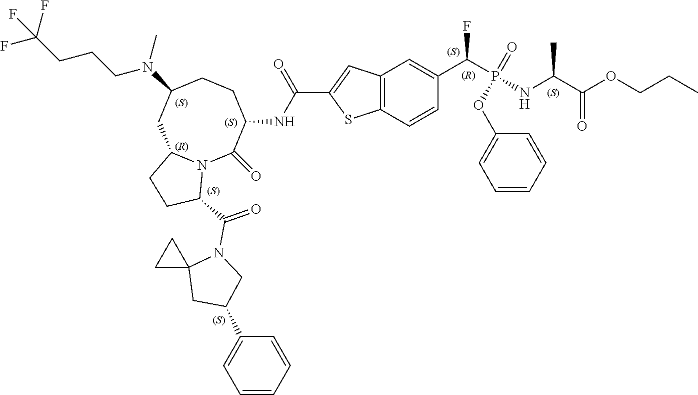 |
| C121 | isopropyl (((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate 2,2,2- trifluoroacetate | 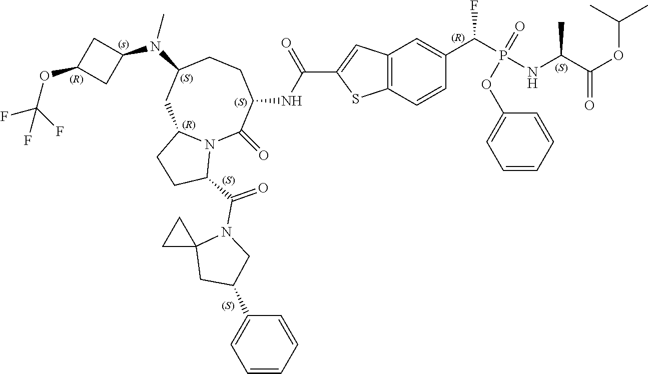 |
| C122 | isobutyl (cyclopropoxy((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) phosphoryl)- L-alaninate | 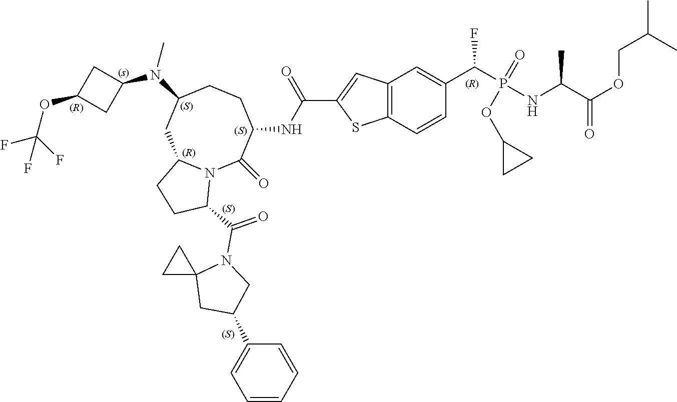 |
| C123 | cyclobutyl (ethoxy((R)- fluoro(2- (((3S,6S,9S,10aR)- 9- (methyl((1s,3R)- 3. (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 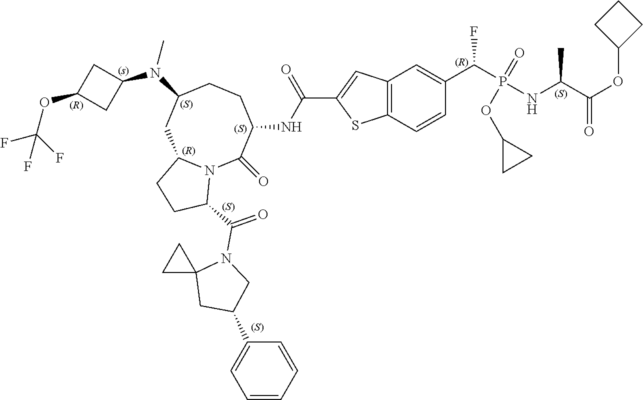 |
| C124 | cyclobutyl ((S)- ((R)-fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 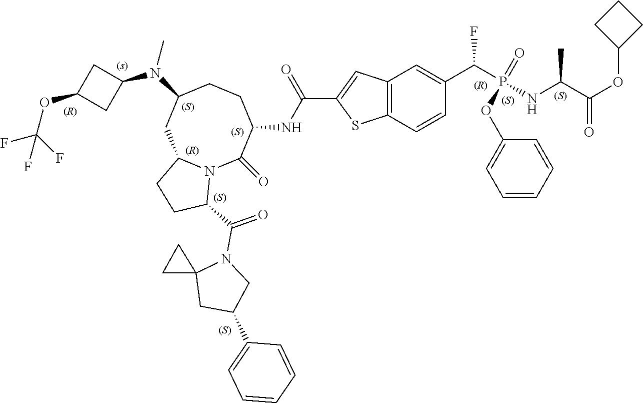 Phosphorus absolute stereochemistry arbitrarily assigned |
| C125 | cyclobutyl ((R)- ((R)-fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 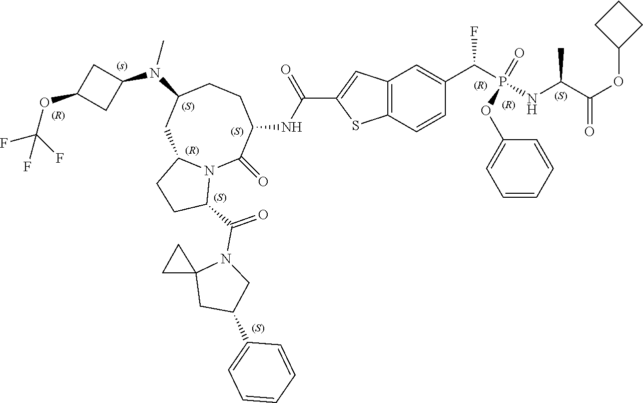 Phosphorus absolute stereochemistry arbitrarily assigned |
| C126 | propyl N-(((R)- fluoro(2- (((3S,6S,9S,10aR)- 9- (methyl((1s,3R)- 3. (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)- O-methyl-L- serinate | 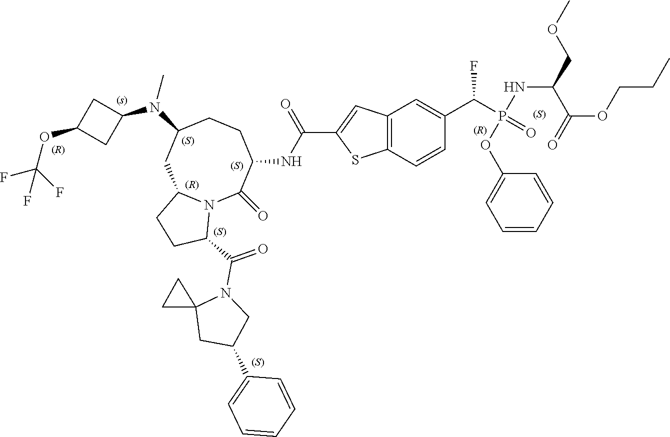 |
| C127 | propyl 2-((((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl) amino)-2- methylpropanoate | 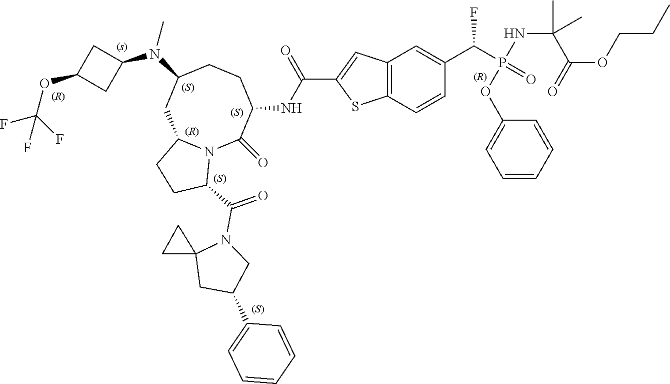 |
| C128 | propyl (((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- phenylalaninate | 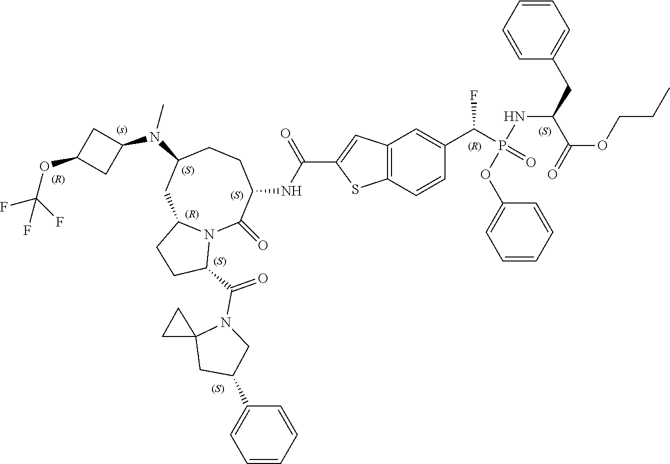 |
| C129 | isobutyl (((R)- fluoro(2- (((3S,6S,9S,10aR)- 9- (methyl(((1s,3R)- 3- (trifluoromethyl) cyclobutyl)methyl) amino)-5-oxo-3- ((S)-6-phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 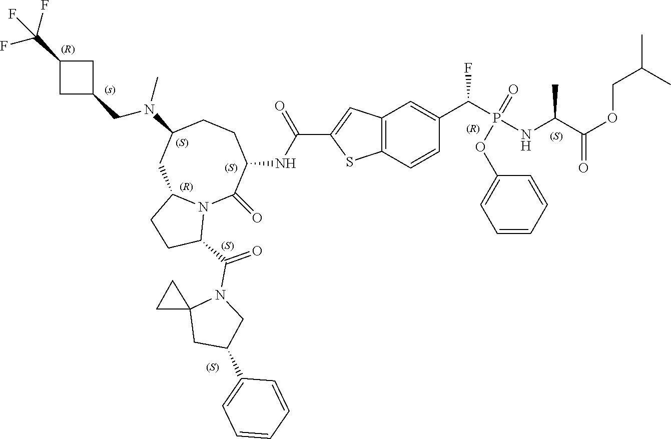 |
| C130 | isobutyl (((R)-(2- (((3S,6S,9S,10aR)- 9-((((1r,3S)-3- (difluoromethyl) cyclobutyl)methyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 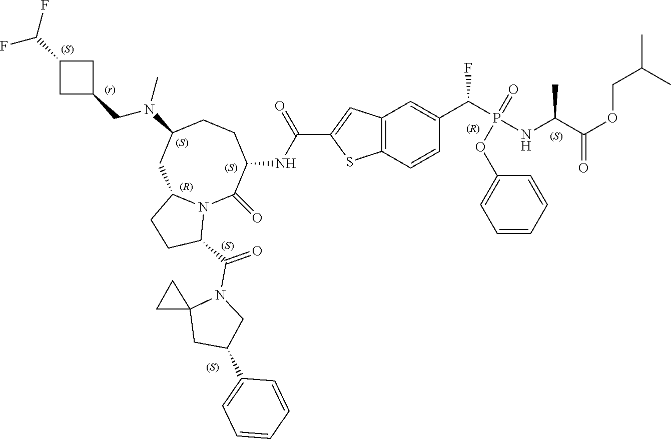 |
| C131 | isobutyl (((R)- fluoro(2- (((3S,6S,9S,10aR)- 9-(methyl(2-(1- (trifluoromethyl)c yclopropyl)ethyl)a mino)-5-oxo-3- ((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 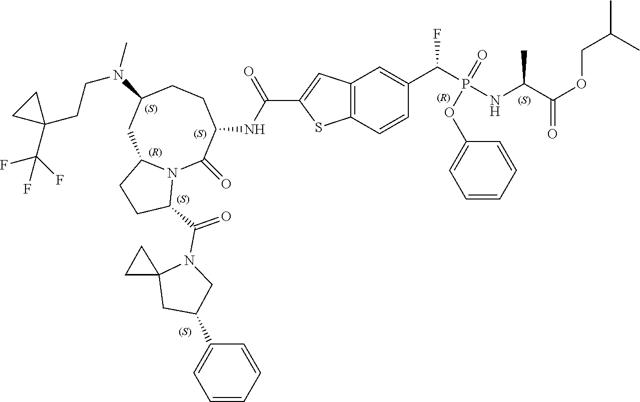 |
| C132 | isobutyl (((R)- fluoro(2- (((3S,6S,9S,10aR)- 9- (methyl((1s,3R)- 3- (trifluoromethoxy) cyclobutyl)amino)- 5-oxo-3-(4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 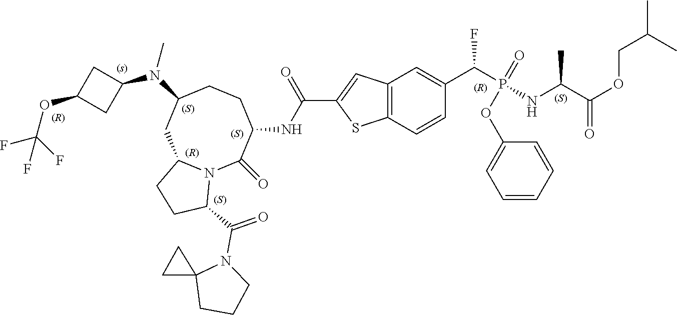 |
| C133 | isobutyl (((R)- fluoro(2- (((3S,6S,9S,10aR)- 9-(methyl(3- (trifluoromethoxy) propyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 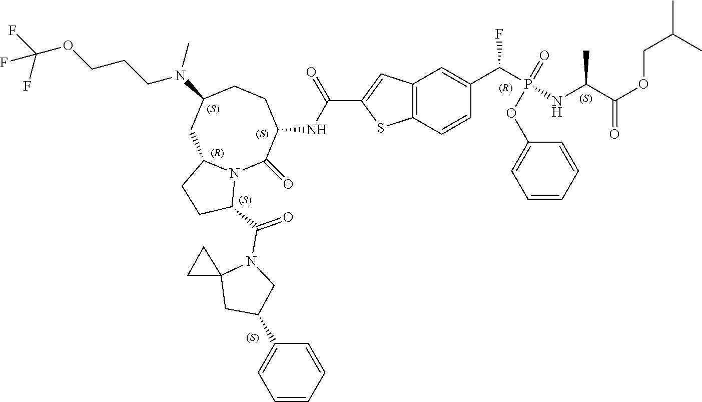 |
| C134 | isobutyl ((R)- ((R)-fluoro(2- (((3S,6S,9S,10aR)- 9- (methyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 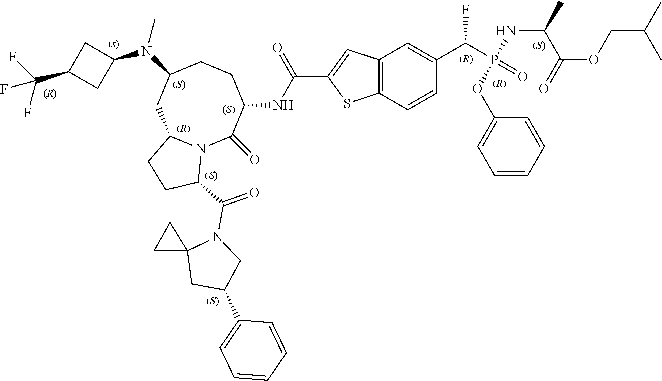 Phosphorus absolute stereochemistry arbitrarily assigned |
| C135 | isobutyl ((S)-((R)- fluoro(2- (((3S,6S,9S,10aR)- 9-(methyl(4,4,4- trifluorobutyl) amino)-5-oxo-3-((S)- 6-phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 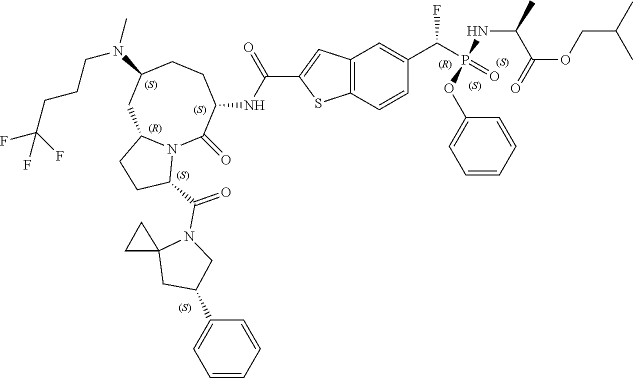 Phosphorus absolute stereochemistry arbitrarily assigned |
| C136 | isobutyl ((R)- ((R)-fluoro(2- (((3S,6S,9S,10aR)- 9-(methyl(4,4,4- trifluorobutyl) amino)-5-oxo-3-((S)- 6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 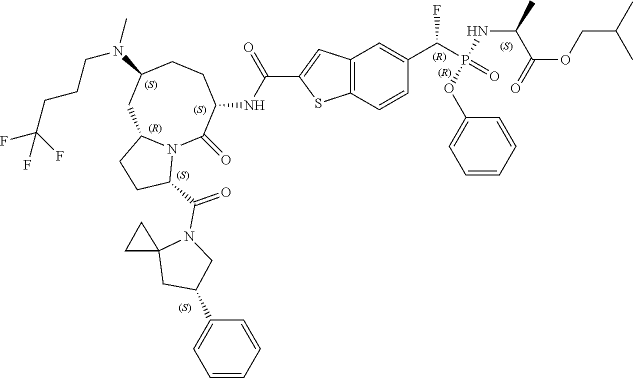 Phosphorus absolute stereochemistry arbitrarily assigned |
| C137 | propyl (((R)- fluoro(2- (((3S,6S,9S,10aR)- 9-(methyl((S)- 4,4,4-trifluoro-3- methoxybutyl) amino)-5-oxo-3-((S)- 6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 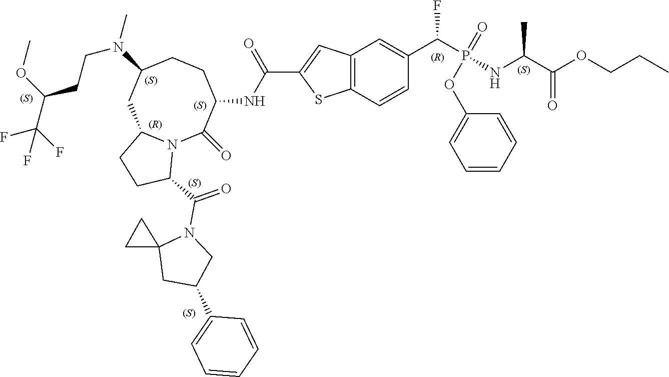 |
| C138 | propyl (((R)-(2- (((3S,6S,9S,10aR)- 9-((((1r,3S)-3- (difluoromethyl) cyclobutyl)methyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 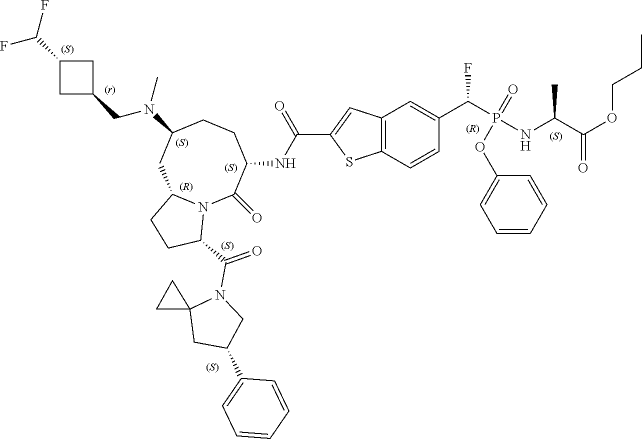 |
| C139 | propyl (((R)- fluoro(2- (((3S,6S,9S,10aR)- 9-(methyl(2-(1- (trifluoromethyl)c yclopropyl)ethyl)a mino)-5-oxo-3- ((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 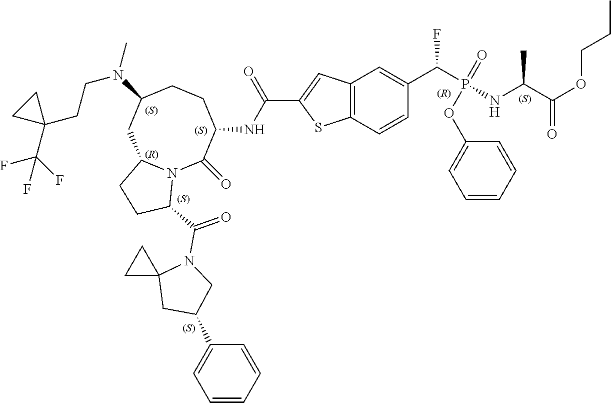 |
| C140 | propyl ((R)-((R)- fluoro(2- (((3S,6S,9S,10aR)- 9-(methyl(3- (trifluoromethoxy) propyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 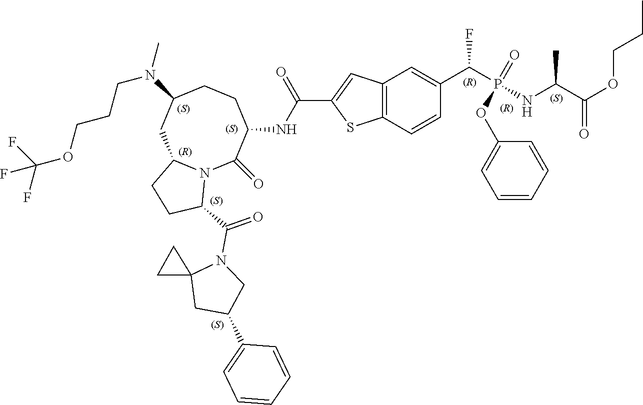 |
| C141 | propyl ((S)-((R)- fluoro(2- (((3S,6S,9S,10aR)- 9-(methyl(3- (trifluoromethoxy) propyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 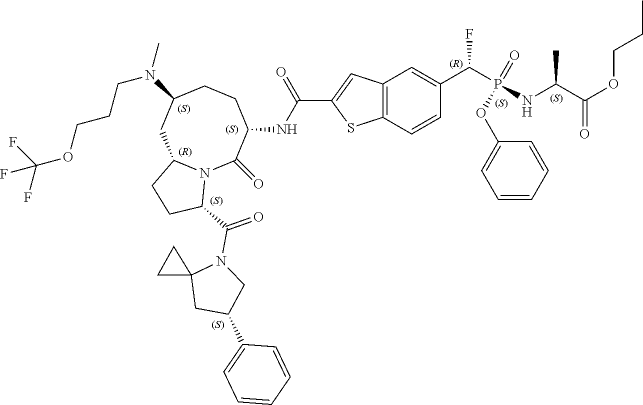 |
| C142 | propyl (((R)-(2- (((3S,6S,9S,10aR)- 9-(((R)-3,3- difluorocyclopent yl)(methyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 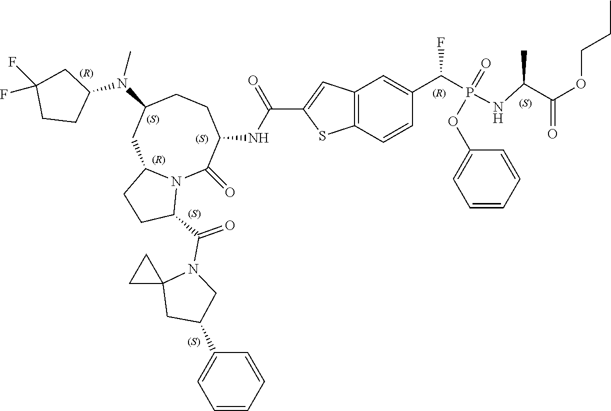 |
| C143 | propyl (((R)-(2- (((3S,6S,9S,10aR)- 9-(((S)-3,3- difluorocyclopent yl)(methyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 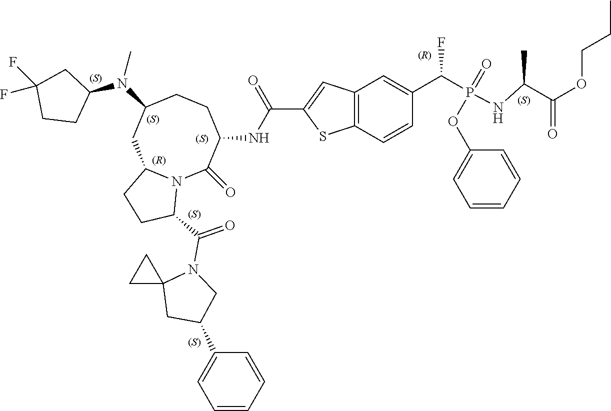 |
| C144 | propyl (((R)- fluoro(2- (((3S,6S,9S,10aR)- 9-(methyl(4,4,4- trifluorobutyl) amino)-5-oxo-3- ((S)- 6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 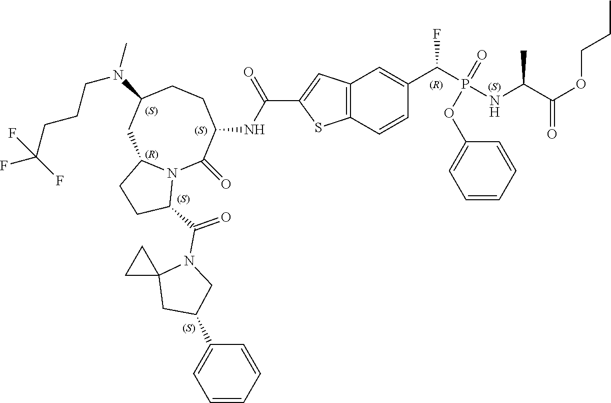 |
| C145 | propyl ((S)-((R)- fluoro(2- (((3S,6S,9S,10aR)- 9-morpholino-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 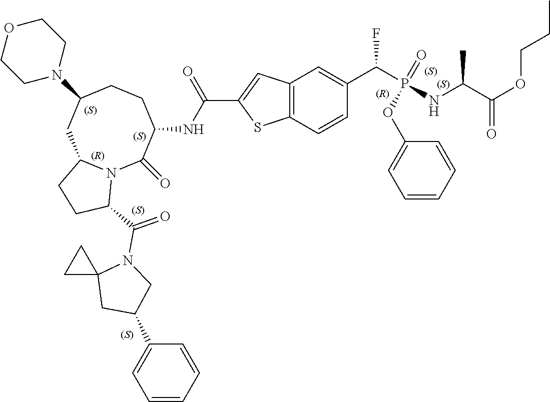 |
| C146 | propyl (((R)- fluoro(2- (((3S,6S,9S,10aR)- 9-(methyl((3- methyloxetan-3- yl)methyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 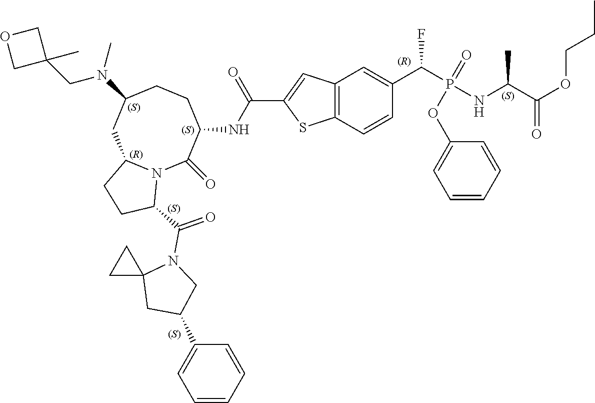 |
| C147 | propyl (((R)- fluoro(2- (((3S,6S,9S,10aR)- 9- (methyl(oxetan-3- yl)amino)-5-oxo- 3-((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 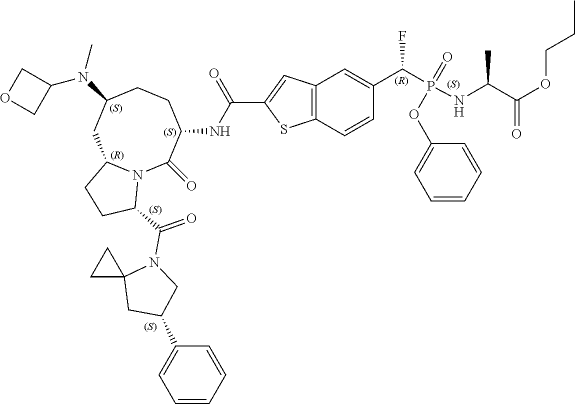 |
| C148 | isobutyl (((R)-(2- (((3S,6S,9S,10aR)- 9-((3,3- difluoropropyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 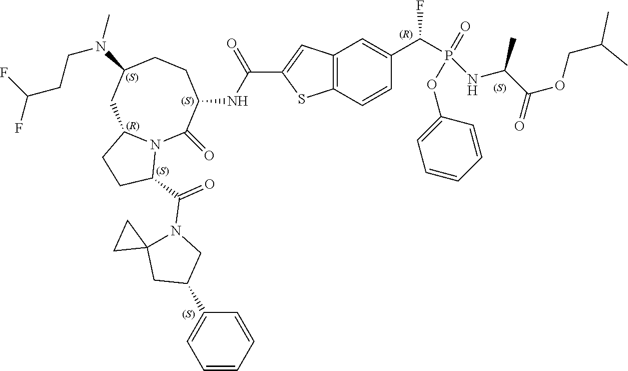 |
| C149 | propyl N-(((R)-(2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- O-methyl-L- serinate | 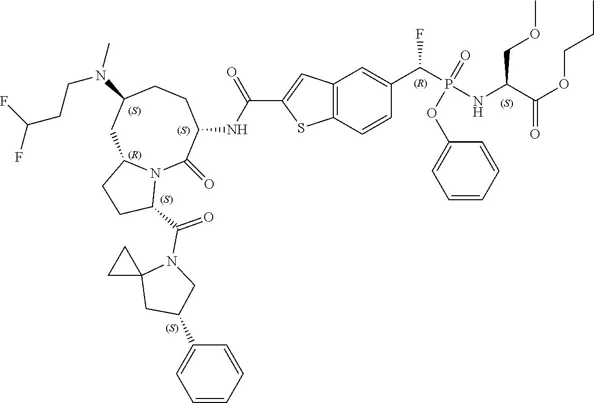 |
| C150 | propyl 2-((((R)- (2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl) amino)-2- methylpropanoate | 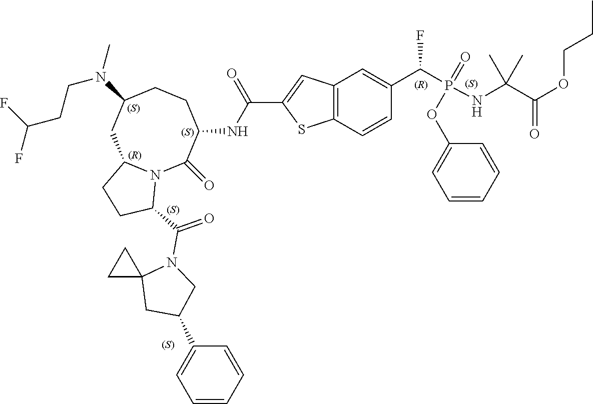 |
| C151 | isopropyl (S)-2- (((S)-((R)-(2- (((3S,6S,9S,10aR)- 9-((3,3- difluoropropyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)fluoromethyl) (phenoxy) phosphoryl) amino) butanoate | 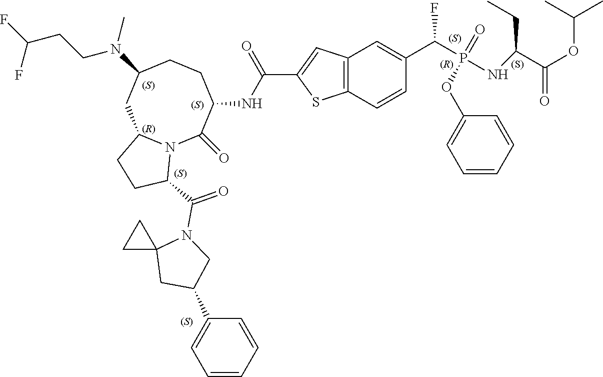 |
| C152 | propyl ((difluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl) prolinate | 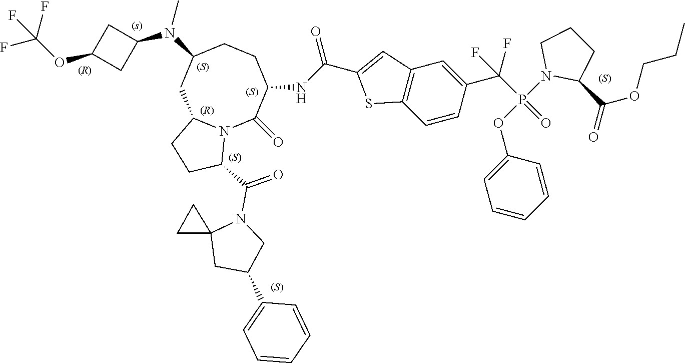 |
| C153 | propyl 1- (ethoxy((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) phosphoryl)- 3,3- difluoropyrrolidine- 2-carboxylate | 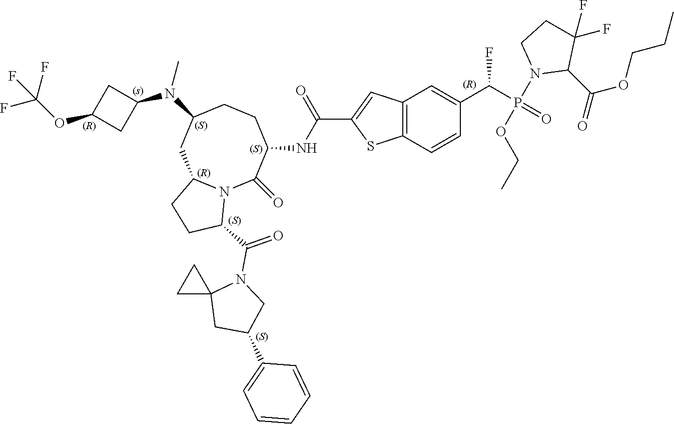 |
| C154 | propyl ((difluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 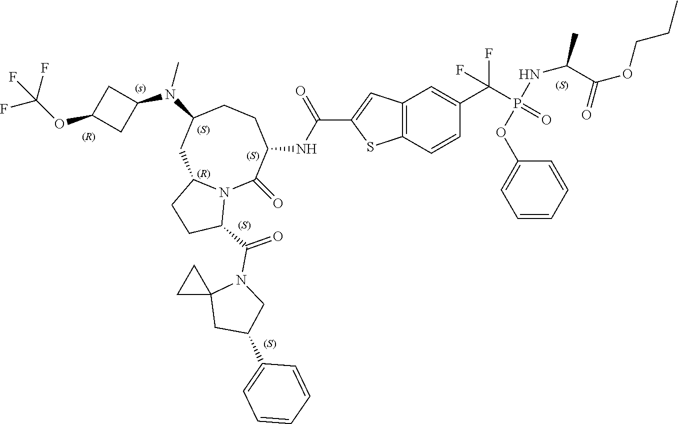 |
| C155 | dipropyl 2,2′- ((((R)-fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl) bis(azanediyl)) (2S,2′S)- dipropionate | 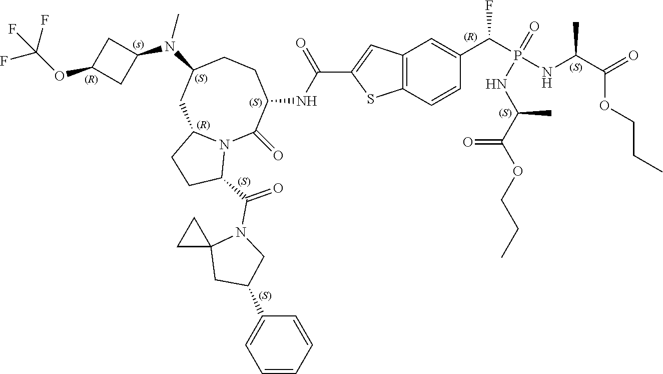 |
| C156 | isobutyl (cyclopropoxy((S)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 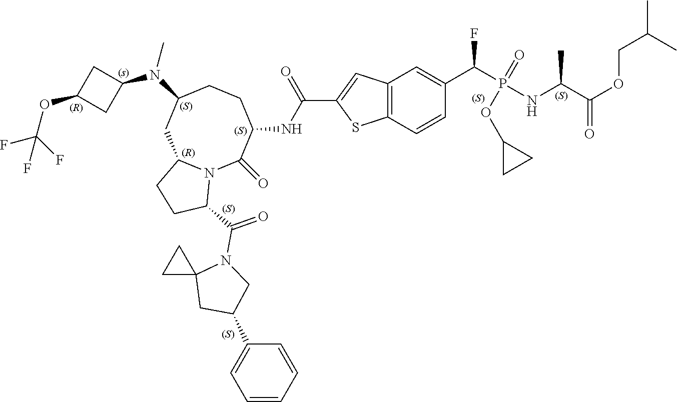 |
| C157 | propyl 1-((((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl) amino) cyclopropane- 1-carboxylate | 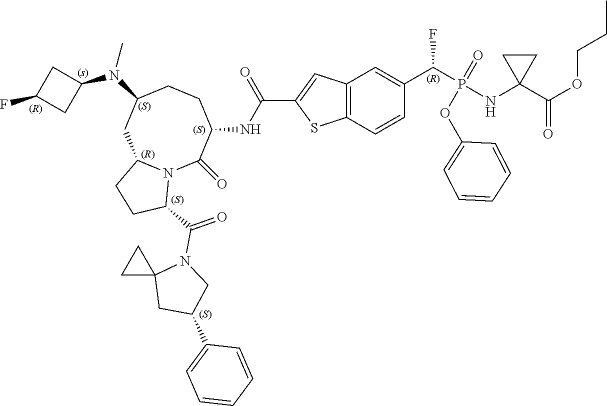 |
| C158 | isobutyl (((R)- fluoro(2- (((3S,6S,9S,10aR)- 9- (methyl((1R,3S)- 3- (trifluoromethoxy) cyclopentyl) amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 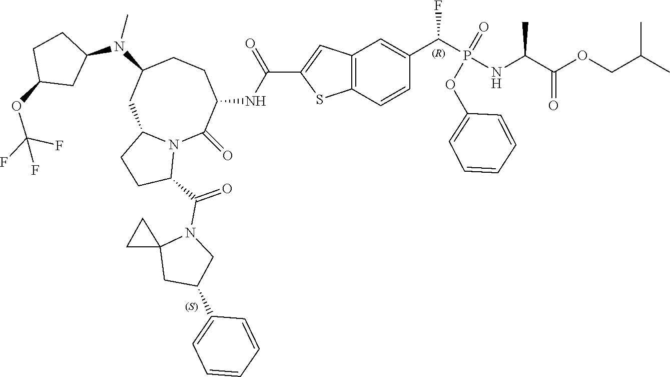 |
| C159 | isobutyl (((S)- fluoro(2- (((3S,6S,9S,10aR)- 9- (methyl((1R,3S)- 3- (trifluoromethoxy) cyclopentyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 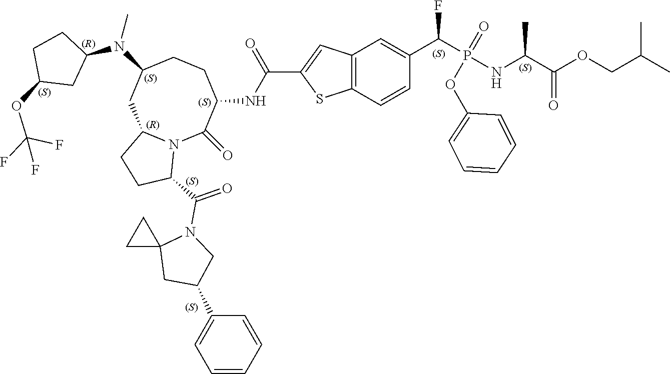 |
| C160 | propyl (((S)- fluoro(2- (((3S,6S,9S,10aR)- 9- (methyl((1R,3S)- 3- (trifluoromethoxy) cyclopentyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 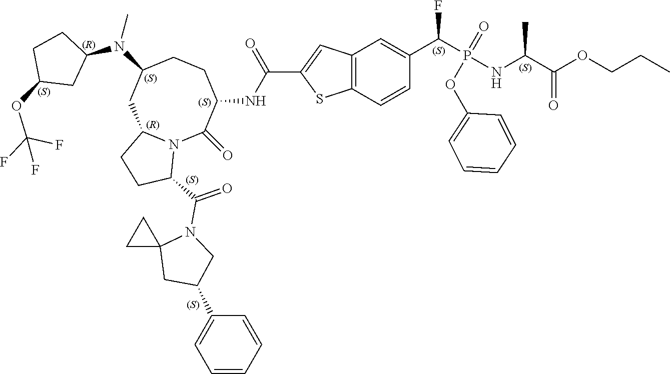 |
| C161 | (1- fluorocyclobutyl) methyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 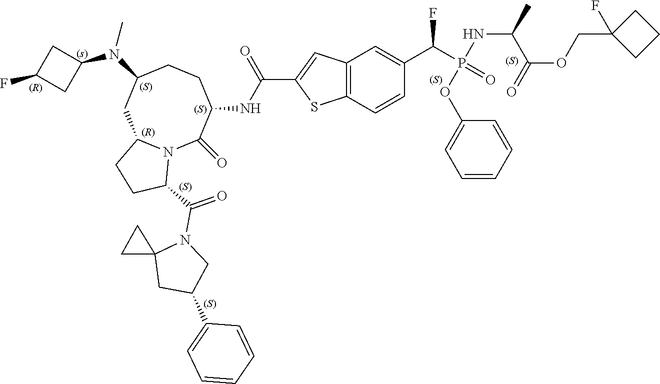 |
| C162 | propyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(3,3,3- trifluoropropoxy) phosphoryl)-L- alaninate | 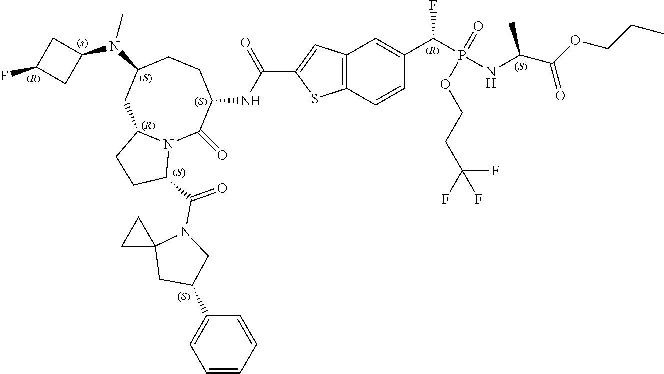 |
| C163 | propyl (((R)- fluoro(2- (((3S,6S,9S,10aR)- 9- (methyl((1s,3R)- 3- (trifluoromethoxy) cyclobutyl)amino)- 5-oxo-3-((R)-6- (pyridin-3-yl)-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 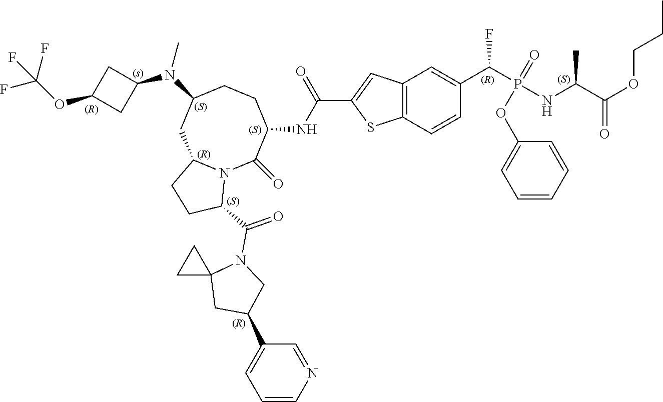 |
| C164 | propyl (((R)- fluoro(2- (((3S,6S,9S,10aR)- 9- (methyl((1s,3R)- 3- (trifluoromethoxy) cyclobutyl)amino)- 5-oxo-3-((S)-6- (pyridin-3-yl)-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 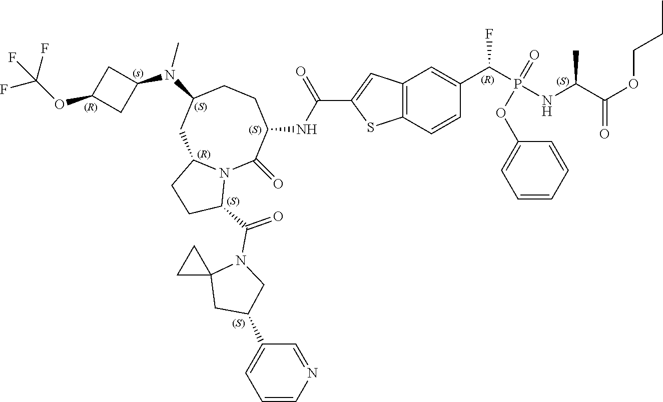 |
| C165 | propyl (((R)- fluoro(2- (((3S,6S,9S,10aR)- 3-((6S,7S)-7- methoxy-6- phenyl-4- azaspiro[2.4] heptane-4-carbonyl)- 9- (methyl((1s,3R)- 3- (trifluoromethoxy) cyclobutyl)amino)- 5- oxodecahydropyrrolo [1,2-a]azocin- 6- yl)carbamoyl) benzo [b]thiophen-5- yl)methyl) (phenoxy) phosphoryl)-L- alaninate | 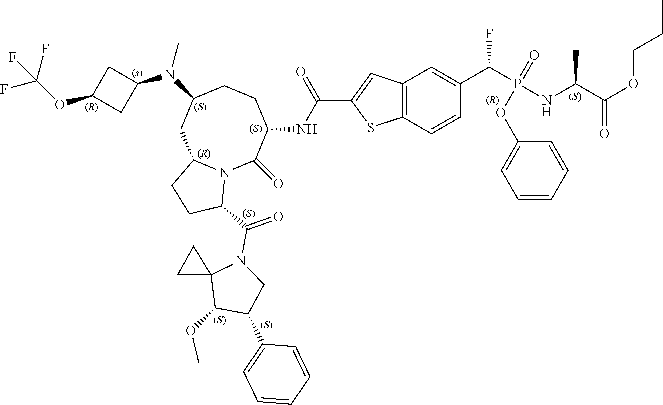 Absolute stereochemistry of cis-3-OMe-4-Ph pyrrolidine arbitrarily assigned |
| C166 | propyl (((R)- fluoro(2- (((3S,6S,9S,10aR)- 3-((6R,7R)-7- methoxy-6- phenyl-4- azaspiro[2.4] heptane-4-carbonyl)- 9- (methyl((1s,3R)- 3- (trifluoromethoxy) cyclobutyl)amino)- 5- oxodecahydropyrrolo [1,2-a]azocin- 6- yl)carbamoyl) benzo [b]thiophen-5- yl)methyl) (phenoxy) phosphoryl)-L- alaninate | 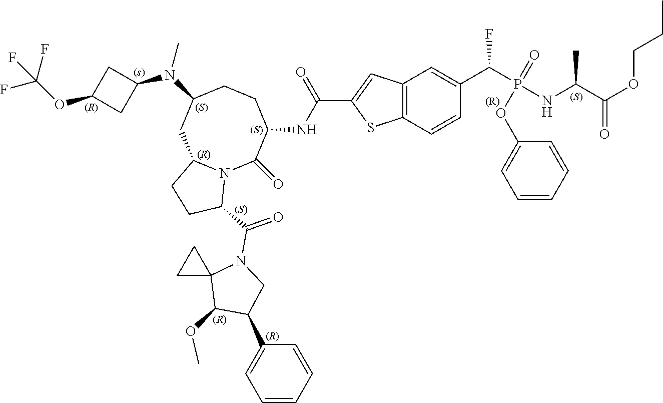 Absolute stereochemistry of cis-3-OMe-4-Ph pyrrolidine arbitrarily assigned |
| C167 | propyl (((R)- fluoro(2- (((3S,6S,9S,10aR)- 9-(methyl((S)- 3,3,3-trifluoro-2- methoxypropyl)a mino)-5-oxo-3- ((S)-6-phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 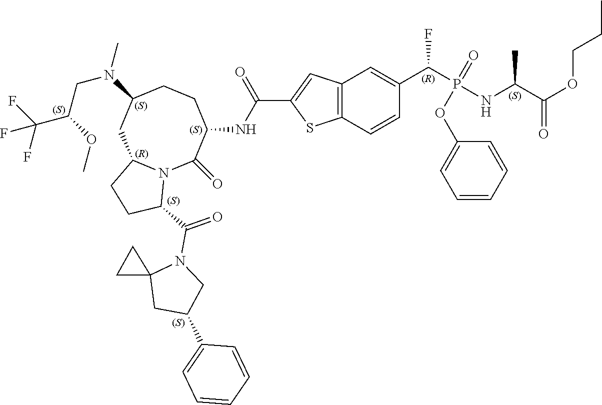 |
| C168 | propyl ((R)-((R)- fluoro(2- (((3S,6S,9S,10aR)- 9-(methyl(2- (trifluoromethoxy) ethyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 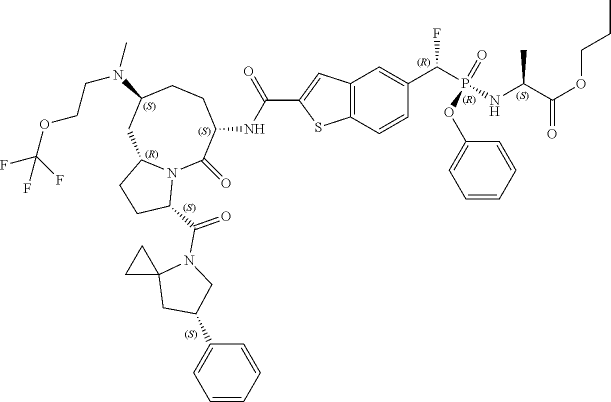 |
| C169 | propyl ((S)-((R)- fluoro(2- (((3S,6S,9S,10aR)- 9-(methyl(2- (trifluoromethoxy) ethyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 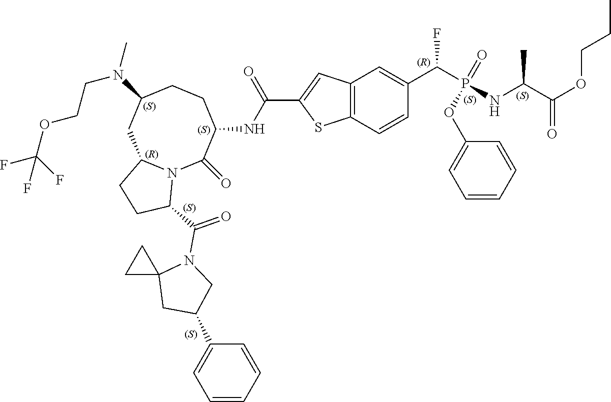 |
| C170 | propyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2- oxopyrrolidin-1- yl)phosphoryl)-L- alaninate | 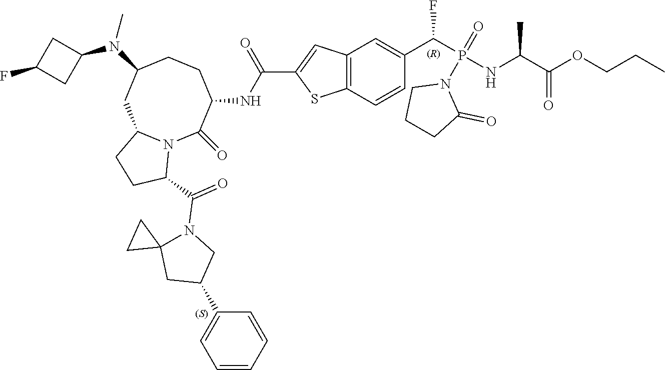 |
| C172 | cyclobutyl ((R)- ((R)-fluoro(2- (((3S,6S,9S,10aR)- 9-(methyl((3- methyloxetan-3- yl)methyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 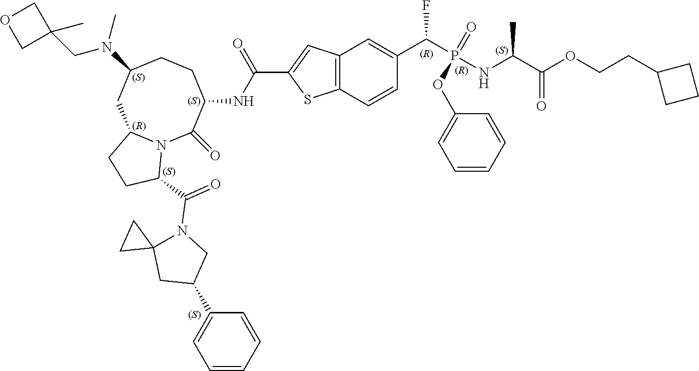 Phosphorus absolute stereochemistry arbitrarily assigned |
| C173 | propyl ((S)-((R)- fluoro(2- (((3S,6S,9S,10aR)- 9-(methyl((3- methyloxetan-3- yl)methyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 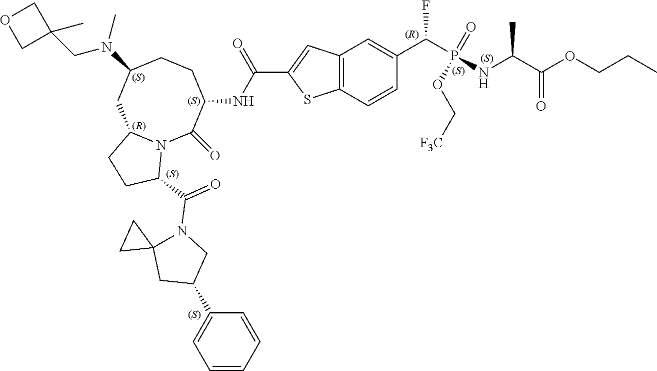 Phosphorus absolute stereochemistry arbitrarily assigned |
| C174 | ((S)- tetrahydrofuran-3- yl)methyl (((R)- fluoro(2- (((3S,6S,9S,10aR)- 9- (methyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 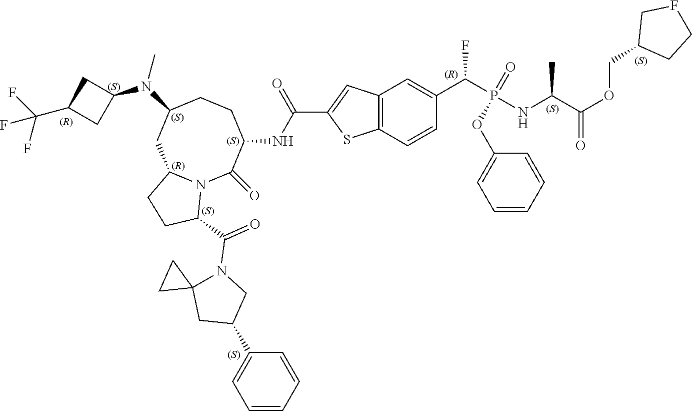 |
| C175 | propyl ((R)-((R)- fluoro(2- (((3S,6S,9S,10aR)- 9- (methyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 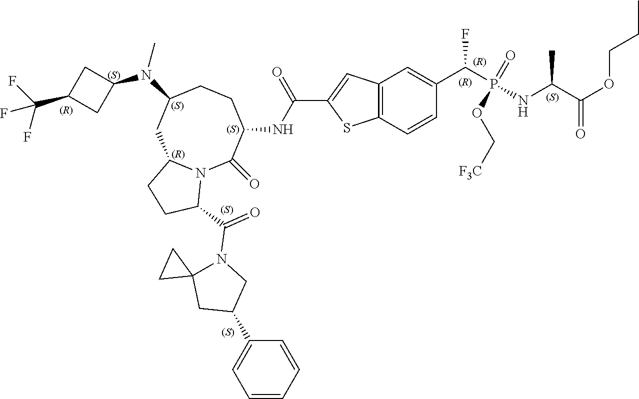 Phosphorus absolute stereochemistry arbitrarily assigned |
| C176 | isopropyl ((R)- ethoxy((R)- fluoro(2- (((3S,6S,9S,10aR)- 9- (methyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 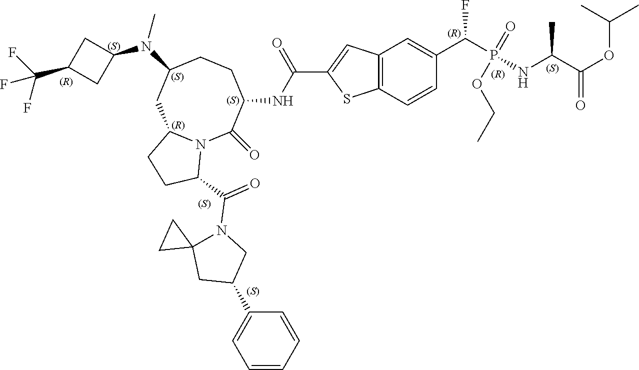 Phosphorus absolute stereochemistry arbitrarily assigned |
| C177 | isopropyl ((S)- ethoxy((R)- fluoro(2- (((3S,6S,9S,10aR)- 9-(methyl(4,4,4- trifluorobutyl) amino)-5-oxo-3-((S)- 6-phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 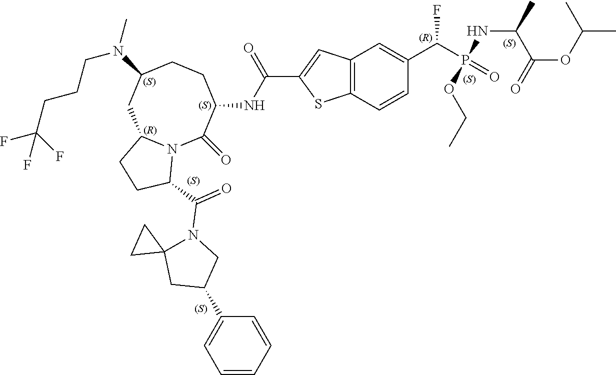 Phosphorus absolute stereochemistry arbitrarily assigned |
| C178 | propyl (ethoxy((R)-(2- (((3S,6S,9S,10aR)- 9-(ethyl((1s,3R)- 3- fluorocyclobutyl)a mino)-5-oxo-3- ((S)-6-phenyl-4- azaspiro[2.4] heptane-4- carbonyl) decahydropyrrolo [1,2- a]azocin-6- yl)carbamoyl) benzo [b]thiophen-5- yl)fluoromethyl) phosphoryl)-L- alaninate | 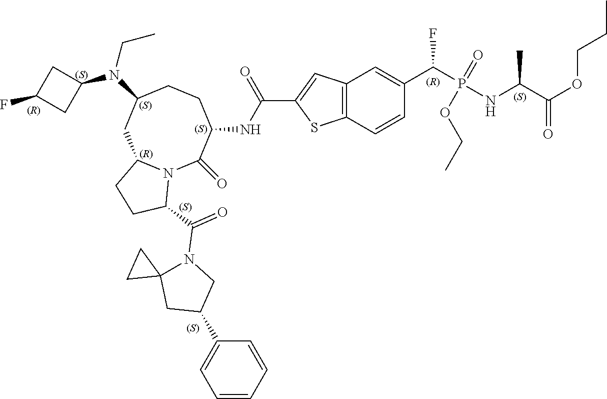 |
| C179 | 2- morpholinoethyl (((R)-(2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 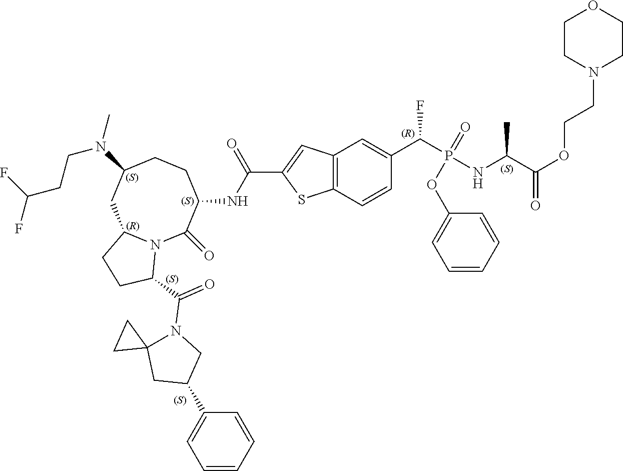 |
| C180 | neopentyl (((R)- (2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-alaninate | 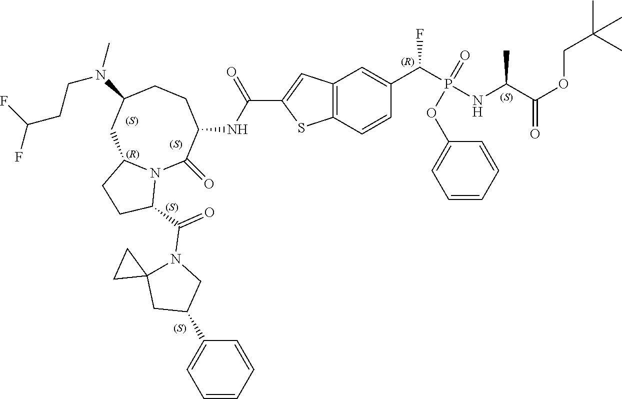 |
| C181 | propyl ((R)-((R)- (2- (((3S,6S,9S,10aR)- 9-((3,3- difluoropropyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)fluoromethyl) (phenoxy) phosphoryl)- L-prolinate | 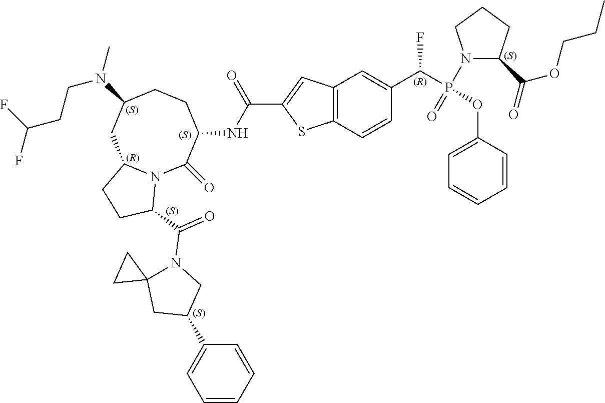 |
| C182 | 2-methoxy-2- methylpropyl (((R)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 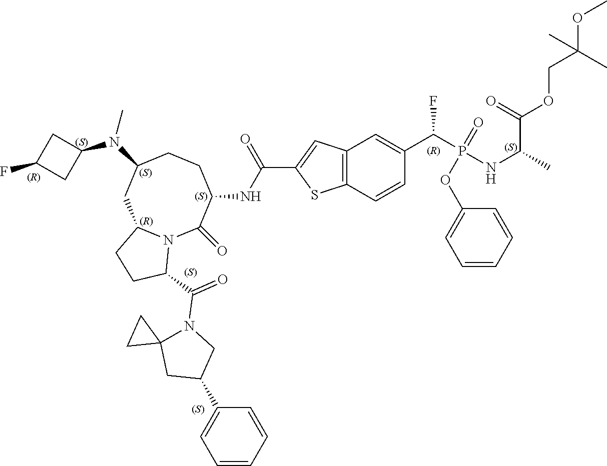 |
| C183 | 6,6- difluorospiro[3.3] heptan-2-yl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 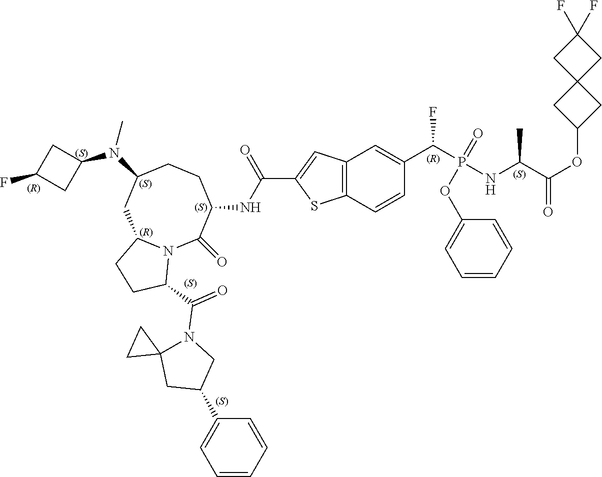 |
| C184 | ((R)- tetrahydrofuran-3- yl)methyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 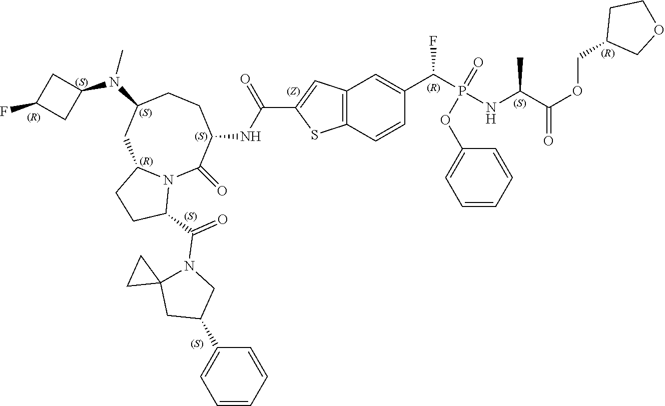 |
| C185 | ((S)- tetrahydrofuran-3- yl)methyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 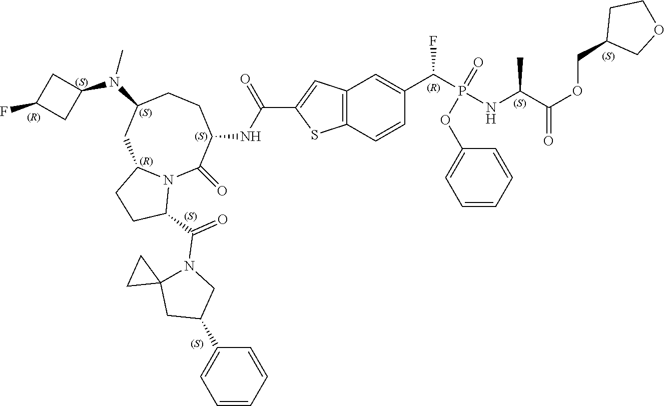 |
| C186 | (tetrahydro-2H- pyran-4-yl)methyl (((R)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 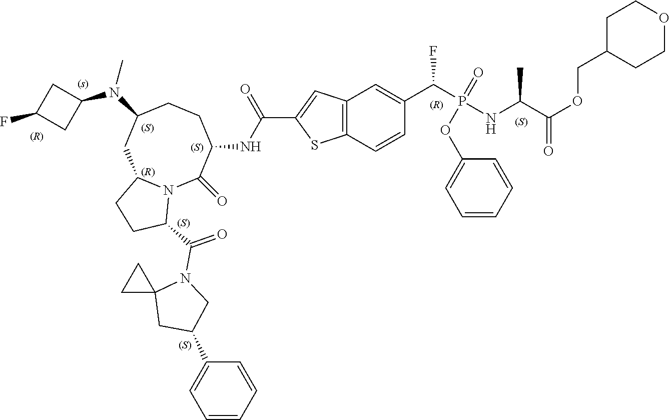 |
| C187 | isobutyl (2S)-2- ((((R)-fluoro(2- (((3S,6S,9S,10aR)- 9-(((1s,3R)-3- fluorocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl) amino) butanoate | 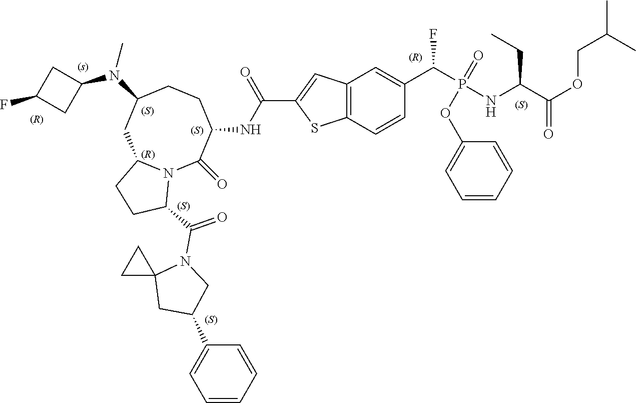 |
| C188 | isopentyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 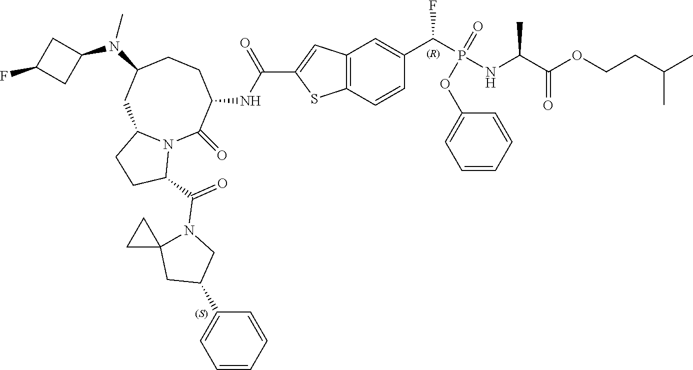 |
| C189 | propyl (2S)-2- ((((R)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl) amino) pentanoate | 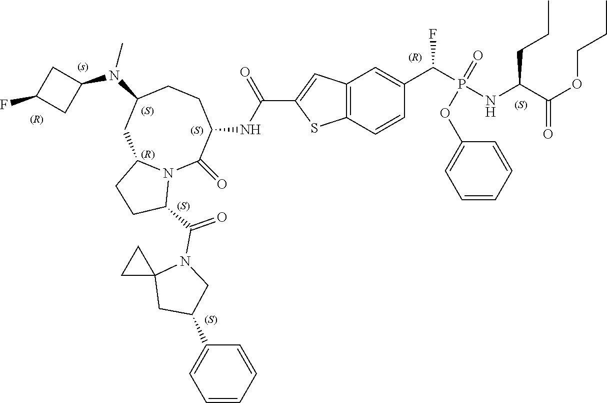 |
| C190 | propyl ((cyclohexyloxy) ((R)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 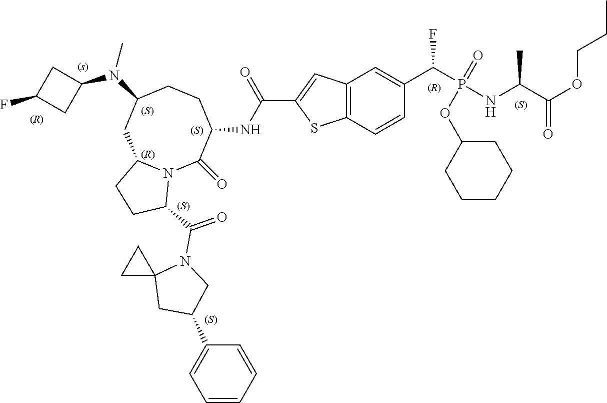 |
| C191 | propyl ((cyclopentyloxy) ((R)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 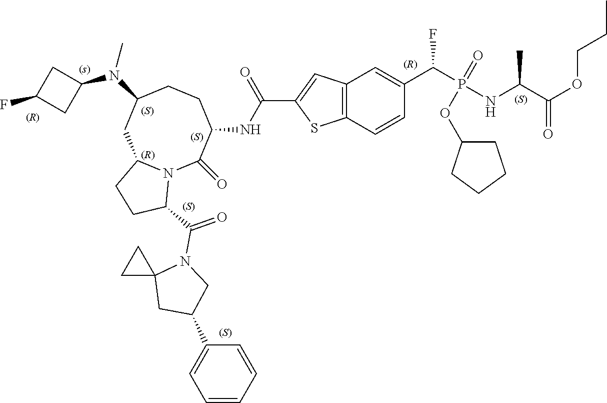 |
| C192 | propyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (isopropoxy) phosphoryl)- L-alaninate | 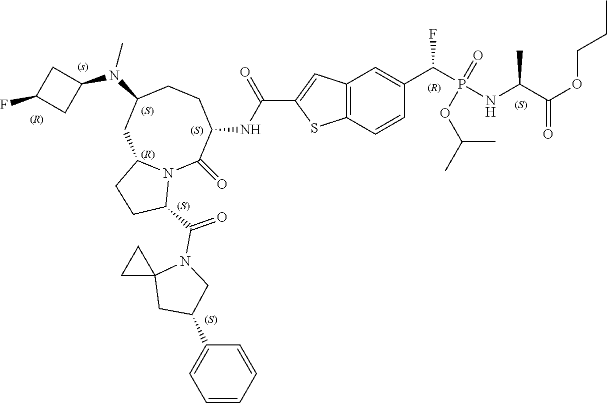 |
| C193 | neopentyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-L- alaninate | 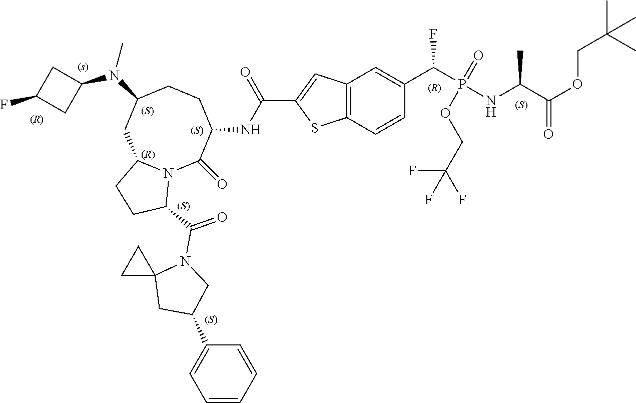 |
| C194 | propyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2- methoxyethoxy) phosphoryl)-L- alaninate | 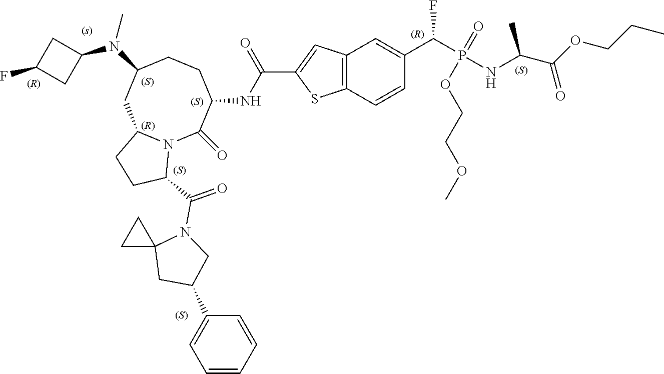 |
| C195 | spiro[3.3]heptan- 2-yl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 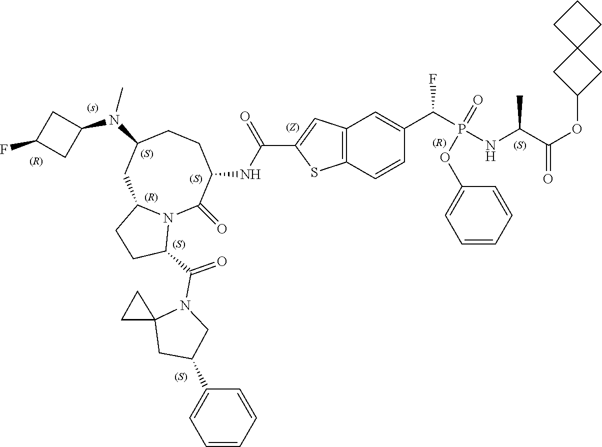 |
| C196 | propyl (((R)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- valinate | 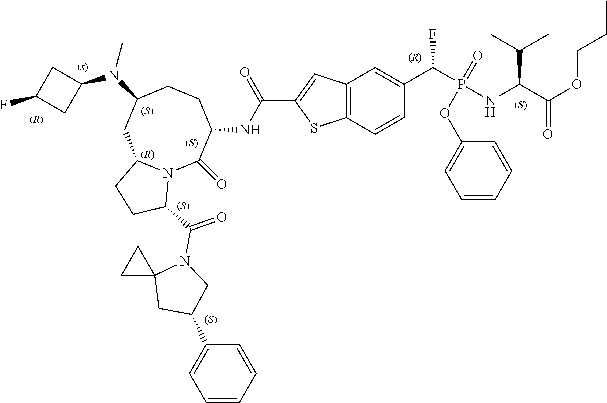 |
| C197 | (R)- tetrahydrofuran-3- yl (((R)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 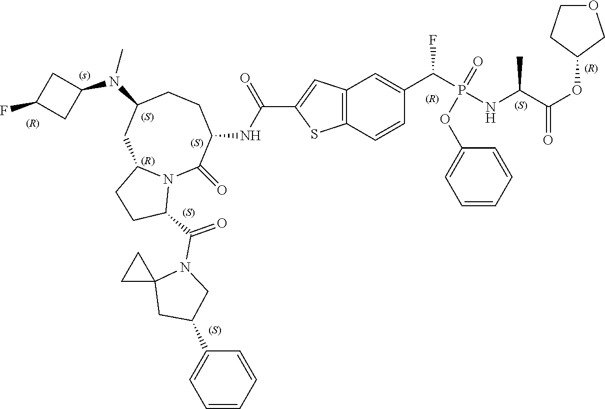 |
| C198 | (S)- tetrahydrofuran-3- yl (((R)-fluoro(2- (((3S,6S,9S,10aR)- 9-(((1s,3R)-3- fluorocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 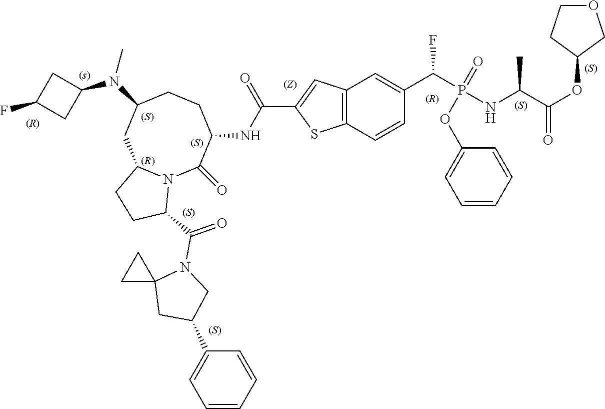 |
| C199 | 2-fluoro-2- methylpropyl (((S)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 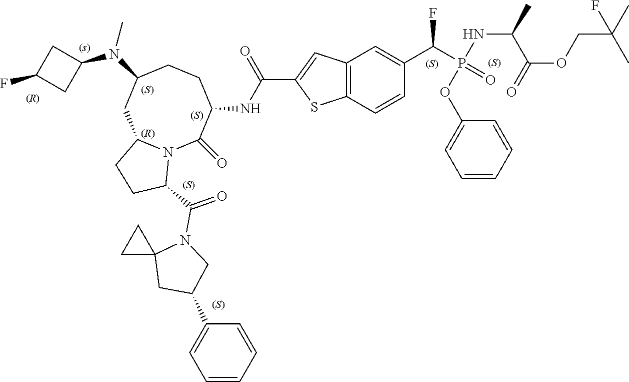 |
| C200 | ((R)- tetrahydrofuran-3- yl)methyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 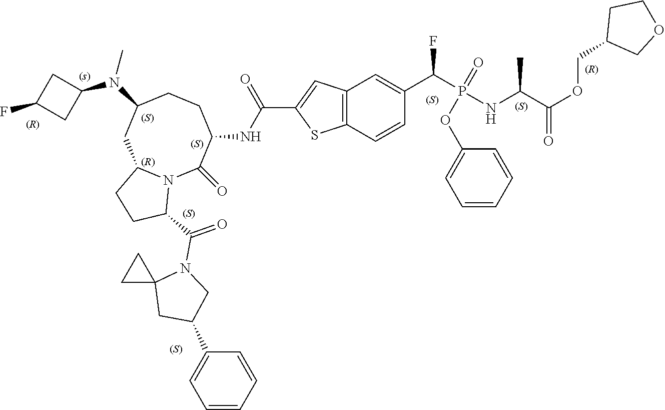 |
| C201 | ((S)- tetrahydrofuran-3- yl)methyl (((S)- fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 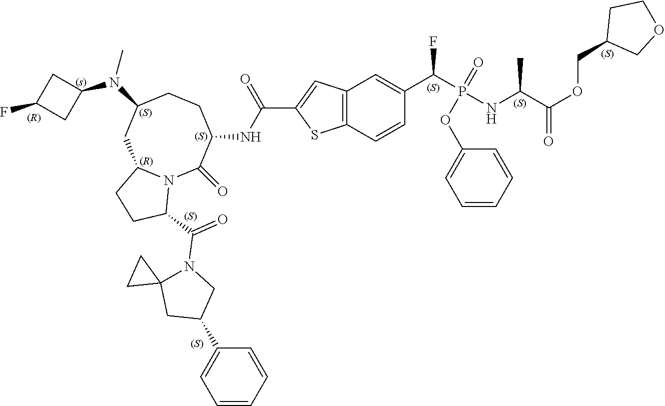 |
| C202 | (tetrahydro-2H- pyran-4-yl)methyl (((S)-fluoro(2- (((3S,6S,9S,10aR)- 9-(((1s,3R)-3- fluorocyclobutyl) (methyl)amino)-5- oxo-3-((S)-6- phenyl-4- azaspiro [2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 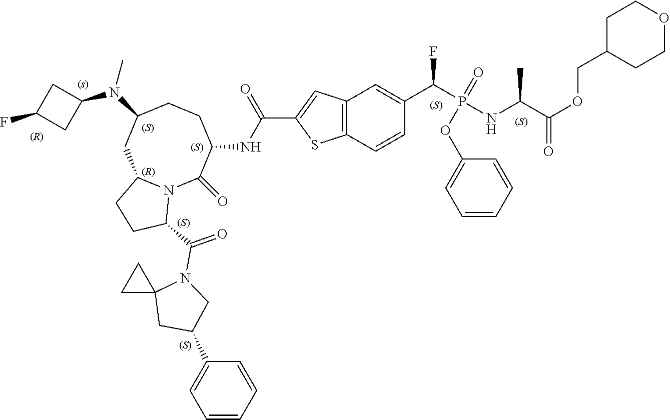 |
| C203 | propyl (2S)-4,4- difluoro-1-(((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl) pyrrolidine-2- carboxylate | 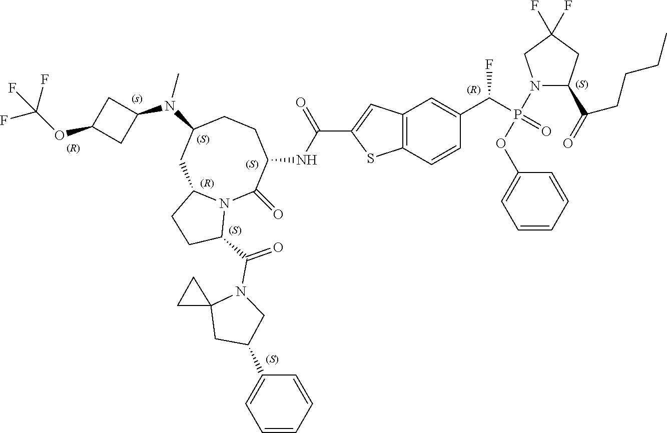 |
| C204 | propyl N-(((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl)(2,2,2- trifluoroethoxy) phosphoryl)-N- methyl-L- alaninate | 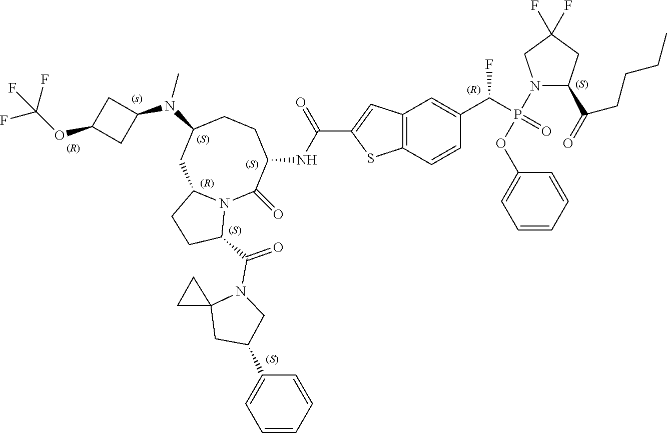 |
| C205 | 2-ethylbutyl (ethoxy((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-alaninate | 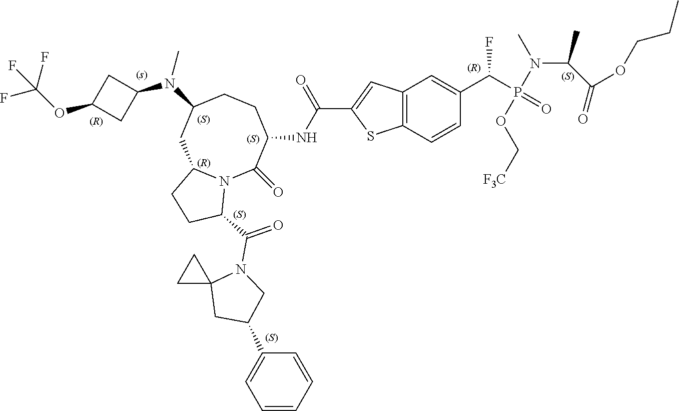 |
| C206 | propyl N- (ethoxy((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- N-methyl-L- alaninate | 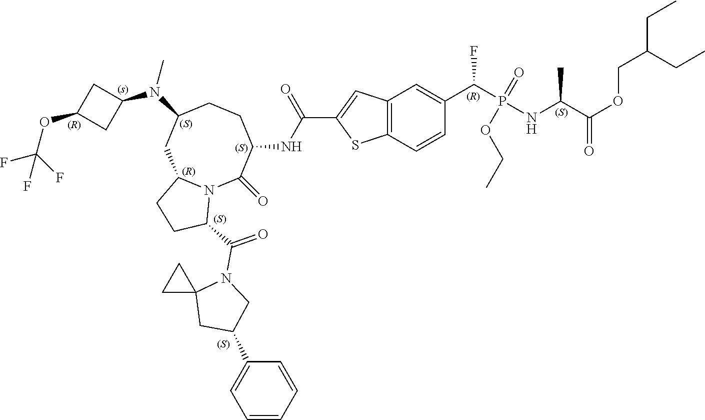 |
| C207 | propyl (ethoxy((R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) phosphoryl)- L-prolinate | 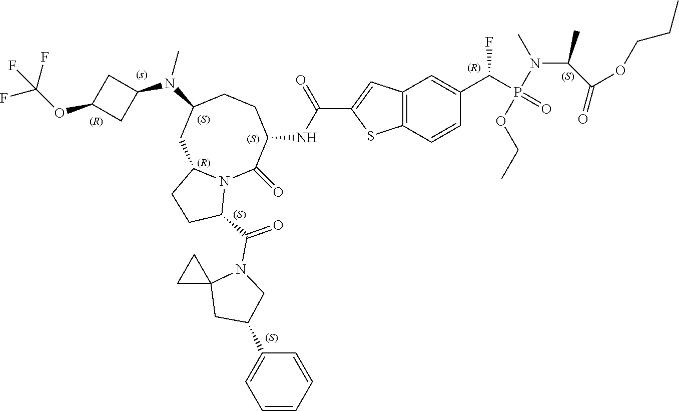 |
| C208 | spiro[3.4]octan-2- yl (((R)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 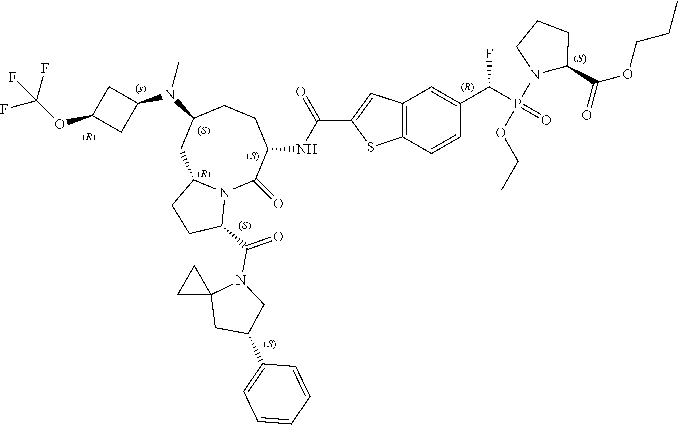 |
| C209 | spiro[2.3]hexan-5- yl (((R)-fluoro(2- (((3S,6S,9S,10aR)- 9-(((1s,3R)-3- fluorocyclobutyl) (methyl)amino)-5- oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 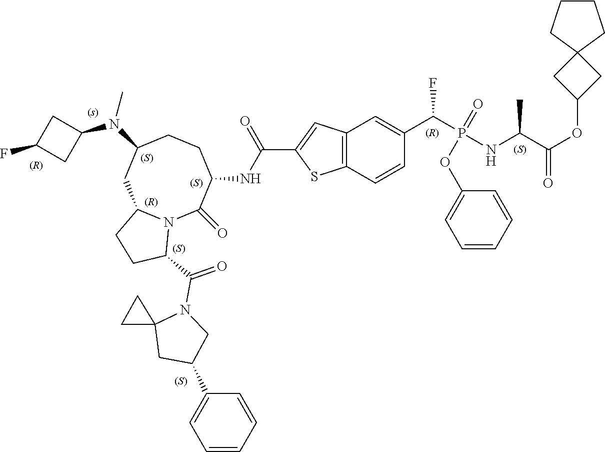 |
| C210 | propyl ((S)-((S)- fluoro(2- (((3S,6S,9S,10aR)- 9- (methyl((1s,3R)- 3- (trifluoromethyl) cyclobutyl)amino)- 5-oxo-3-((S)-6- phenyl-4- azaspiro[2.4] heptane-4- carbonyl) decahydropyrrolo [1,2- a]azocin-6- yl)carbamoyl) benzo [b]thiophen-5- yl)methyl) (phenoxy) phosphoryl)-L- alaninate | 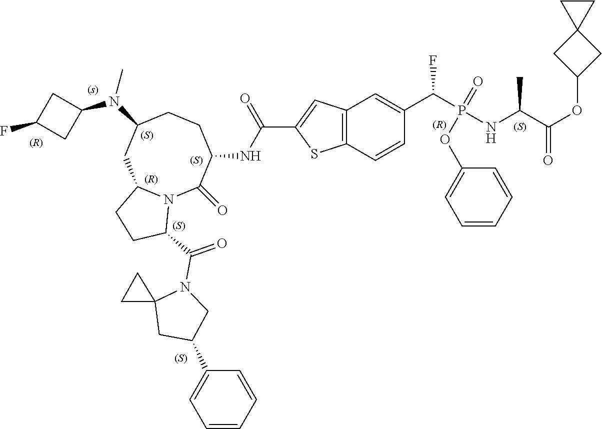 |
| C211 | propyl 2-((((1R)- fluoro(2- (((3R,6R,9R,10aS)- 9- (methyl((1s,3S)- 3- (trifluoromethoxy) cyclobutyl)amino)- 5-oxo-3-((R)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)methyl)(isobut oxy)phosphoryl) amino)-2- methylpropanoate | 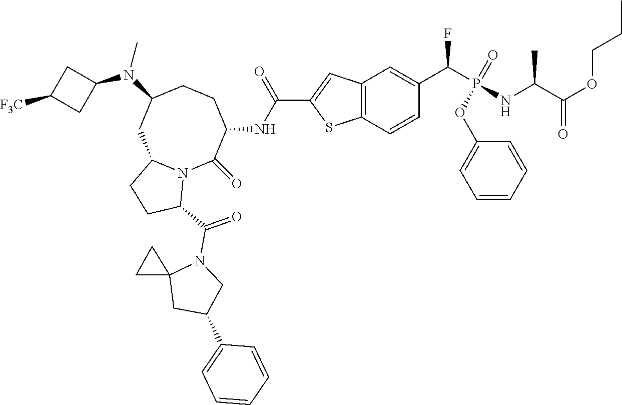 [SMILES: CCCOC(=O)[C@H](C)N[P@@](=O)(Oc1ccccc1)[C@H](F)c1ccc2sc(C(=O)N[C@H]3CC[C@H](N(C)[C@H]4C[C@@H](C(F)(F)F)C4)C[C@H]4CC[C@@H](C(=O)N5C[C@H](c6ccccc6)CC56CC6)N4C3=O)cc2c1] |
| C212 | propyl 2-((((1R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)methyl) (isopropoxy) phosphoryl) amino)-2- methylpropanoate | 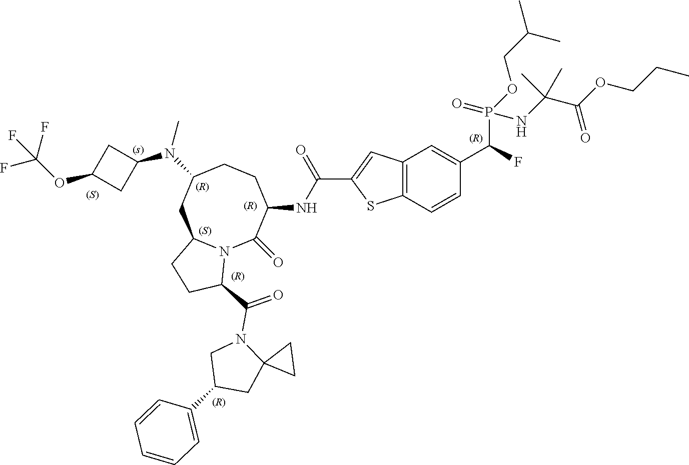 |
| C213 | propyl 2- ((butoxy((1S)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)methyl) phosphoryl) amino)-2- methylpropanoate | 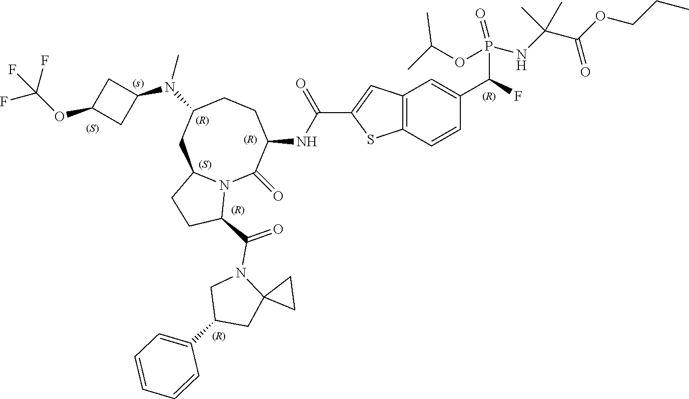 |
| C214 | propyl 2-((((1R)- fluoro(2- (((3S,6S,9S,10aR)- 9- (methyl((1s,3R)- 3- (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)methyl) (propoxy) phosphoryl) amino)-2- methylpropanoate | 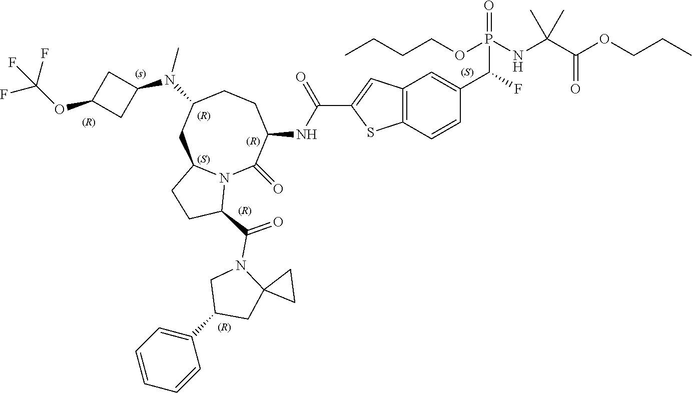 |
| C215 | propyl 2- ((ethoxy((1R)- fluoro(2- (((3S,6S,9S,10aR)- 9- (methyl((1s,3R)- 3. (trifluoromethoxy) cyclobutyl)amino)- 5-oxo-3-((S)-6- phenyl-4- azaspiro[2.4] heptane-4- carbonyl) decahydropyrrolo [1,2- a]azocin-6- yl)carbamoyl)- 2,3- dihydrobenzo[b] thiophen-5- yl)methyl) phosphoryl) amino)-2- methylpropanoate | 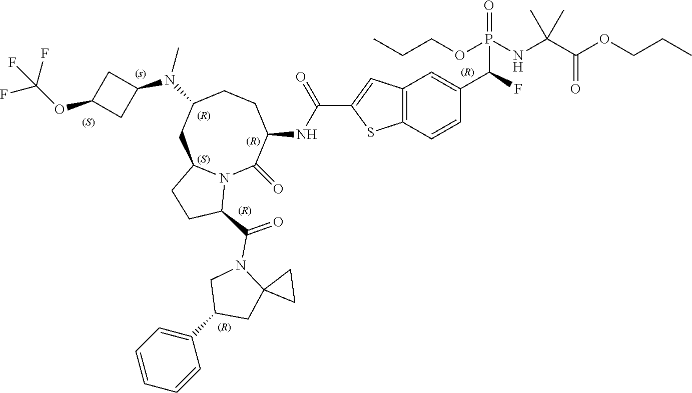 |
| | No. | NMR and LCMS |
| | C1 | 1H NMR (400 MHz, DMSO-d6) δ (ppm) 8.85-8.69 (m, 1H), 8.27 (s, 1H), 8.12-7.97 (m, 2H), 7.65- 7.52 (m, 1H), 7.39- 7.12 (m, 10H), 6.27-5.87 (m, 3H), 4.84-4.71 (m, 1H), 4.54- 4.40 (m, 1H), 4.36- 4.23 (m, 1H), 4.16-4.05 (m, 1H), 4.00-3.68 (m, 4H), 3.62- 3.44 (m, 1H), 3.04- 2.91 (m, 1H), 2.48-1.34 (m, 23H), 1.17-1.05 (m, 3H), 0.85- 0.75 (m, 3H), 0.52- 0.41 (m, 2H); 950.5 [M + H]⁺ . |
| | C2 | 1H NMR (400 MHz, DMSO-d6) δ (ppm) 8.86-8.71 (m, 1H), 8.27 (s, 1H), 8.12-8.01 (m, 2H), 7.65- 7.56 (m, 1H), 7.40- 7.28 (m, 6H), 7.21-7.08 (m, 4H), 6.29-6.06 (m, 2H), 4.76- 4.66 (m, 1H), 4.48 (t, J = 8.6 Hz, 1H), 4.35-4.26 (m, 1H), 4.12 (t, J = 8.5 Hz, 1H), 3.96- 3.89 (m, 1H), 3.85- 3.67 (m, 3H), 3.58-3.49 (m, 1H), 3.06-2.89 (m, 2H), 2.77 (s, 1H), 2.58 (s, 2H), 2.41-2.20 (m, 3H), 2.12-2.06 (m, 1H), 2.03- 1.89 (m, 6H), 1.88- 1.76 (m, 2H), 1.74-1.40 (m, 8H), 1.16-1.08 (m, 3H), 0.85- 0.75 (m, 3H), 0.59- 0.39 (m, 2H); |
| | C3 | 963.0 [M + H]⁺ . 1H NMR (400 MHz, DMSO-d6) δ (ppm) 10.20-9.71 (m, 1H), 8.91- 8.72 (m, 1H), 8.32- 8.20 (m, 1H), 8.13-8.01 (m, 2H), 7.66-7.55 (m, 1H), 7.39- 7.28 (m, 6H), 7.25- 7.10 (m, 4H), 6.31-6.06 (m, 2H), 4.85-4.77 (m, 1H), 4.62- 4.47 (m, 2H), 4.22- 4.01 (m, 1H), 3.89-3.76 (m, 5H), 3.65-3.50 (m, 2H), 3.23- 2.83 (m, 4H), 2.69- 2.60 (m, 3H), 2.48-2.40 (m, 1H), 2.32-2.16 (m, 2H), 2.12- 1.97 (m, 5H), 1.95- 1.59 (m, 5H), 1.57-1.40 (m, 3H), 1.20-0.91 (m, 3H), 0.81 (t, J = 7.4 Hz, 3H), 0.63- 0.44 (m, 2H); 962.8 [M + H]⁺ . |
| | C4 | 1H NMR (400 MHz, DMSO-d6) δ (ppm) 8.85-8.70 (m, 1H), 8.27 (s, 1H), 8.09-8.03 (m, 2H), 7.61 (d, J = 8.5 Hz, 1H), 7.39- 7.26 (m, 6H), 7.24-7.12 (m, 4H), 6.27-6.04 (m, 2H), 4.77- 4.64 (m, 1H), 4.52- 4.43 (m, 1H), 4.35-4.23 (m, 1H), 4.16-4.06 (m, 1H), 3.88- 3.68 (m, 4H), 3.60- 3.48 (m, 1H), 3.08-2.90 (m, 2H), 2.84-2.70 (m, 1H), 2.39- 2.18 (m, 3H), 2.15- 2.04 (m, 3H), 2.02-1.90 (m, 6H), 1.89-1.48 (m, 9H), 1.46- 1.36 (m, 2H), 1.13 (d, J = 7.1 Hz, 3H), 0.78 (t, J = 7.4 Hz, 3H), 0.53-0.38 (m, 2H); 994.3 [M + H]⁺ . |
| | C5 | 1H NMR (400 MHz, DMSO-d6) δ (ppm) 8.79 (d, J = 7.7 Hz, 1H), 8.27 (s, 1H), 8.10-8.01 (m, 2H), 7.61 (d, J = 8.6 Hz, 1H), 7.38- 7.27 (m, 6H), 7.24-7.13 (m, 4H), 6.26-6.06 (m, 2H), 4.71 (t, J = 8.8 Hz, 1H), 4.57- 4.44 (m, 2H), 4.34-4.24 (m, 1H), 4.12 (t, J = 8.6 Hz, 1H), 3.88- 3.70 (m, 4H), 3.59- 3.49 (m, 1H), 3.05-2.94 (m, 1H), 2.71-2.58 (m, 2H), 2.45- 2.30 (m, 2H), 2.23 (t, J = 11.6 Hz, 1H), 2.18-2.08 (m, 2H), 2.01- 1.91 (m, 6H), 1.90- 1.49 (m, 9H), 1.46-1.36 (m, 2H), 1.13 (d, J = 7.1 Hz, 3H), 0.78 (t, J = 7.4 Hz, 3H), 0.53-0.39 (m, 2H); 1010.8 [M + H]⁺ . |
| | C6 | 1H NMR (400 MHz, DMSO-d6) δ (ppm) 8.80 (d, J = 7.7 Hz, 1H), 8.27 (s, 1H), 8.12-8.03 (m, 2H), 7.60 (d, J = 8.6 Hz, 1H), 7.39- 7.27 (m, 6H), 7.24-7.15 (m, 4H), 6.27-6.11 (m, 2H), 4.71 (t, J = 8.9 Hz, 1H), 4.57- 4.44 (m, 2H), 4.35-4.24 (m, 1H), 4.12 (t, J = 8.6 Hz, 1H), 3.99- 3.88 (m, 2H), 3.82- 3.69 (m, 2H), 3.60-3.48 (m, 1H), 3.07-2.96 (m, 1H), 2.72- 2.58 (m, 2H), 2.46- 2.30 (m, 2H), 2.23 (t, J = 11.6 Hz, 1H), 2.17- 2.08 (m, 2H), 2.03- 1.91 (m, 6H), 1.91-1.45 (m, 11H), 1.13 (d, J = 7.1 Hz, 3H), 0.83 (t, J = 7.4 Hz, 3H), 0.57-0.36 (m, 2H); 1011.3 [M + H]⁺ . |
| | C7 | 1H NMR (400 MHz, DMSO-d6) δ (ppm) 8.85-8.74 (m, 1H), 8.31- 8.23 (m, 1H), 8.09- 8.00 (m, 2H), 7.66-7.55 (m, 1H), 7.39-7.09 (m, 10H), 6.32- 6.12 (m, 2H), 4.76- 4.65 (m, 1H), 4.58-4.43 (m, 2H), 4.33-4.23 (m, 1H), 4.16- 4.08 (m, 1H), 3.96- 3.82 (m, 3H), 3.79-3.69 (m, 1H), 3.60-3.49 (m, 1H), 3.06- 2.94 (m, 1H), 2.73- 2.57 (m, 2H), 2.44-2.11 (m, 5H), 1.99-1.42 (m, 17H), 1.21- 1.12 (m, 3H), 0.85- 0.76 (m, 3H), 0.54-0.35 (m, 2H); 1010.7 [M + H]⁺ . |
| | C8 | 1H NMR (400 MHz, DMSO-d6) δ (ppm) 8.78 (d, J = 7.5 Hz, 1H), 8.27 (s, 1H), 8.12-8.01 (m, 2H), 7.60 (d, J = 8.6 Hz, 1H), 7.40- 7.26 (m, 6H), 7.24-7.15 (m, 4H), 6.28-6.10 (m, 2H), 4.85- 4.72 (m, 1H), 4.47 (t, J = 8.6 Hz, 1H), 4.36-4.24 (m, 1H), 4.16-4.05 (m, 1H), 3.99- 3.88 (m, 2H), 3.82- 3.68 (m, 2H), 3.59-3.47 (m, 1H), 3.00-2.88 (m, 1H), 2.47- 2.08 (m, 10H), 2.02- 1.47 (m, 15H), 1.13 (d, J = 7.1 Hz, 3H), 0.83 (t, J = 7.4 Hz, 3H), 0.55- 0.38 (m, 2H); 982.5 [M + H]⁺ . |
| | C9 | 1H NMR (400 MHz, DMSO-d6) δ (ppm) 8.92-8.67 (m, 1H), 8.32- 8.23 (m, 1H), 8.13- 8.02 (m, 2H), 7.64-7.56 (m, 1H), 7.42-7.26 (m, 6H), 7.25- 7.05 (m, 4H), 6.34- 5.98 (m, 3H), 4.93-4.71 (m, 1H), 4.64-4.35 (m, 2H), 4.19- 4.07 (m, 1H), 4.01- 3.79 (m, 3H), 3.77-3.62 (m, 2H), 3.59-3.46 (m, 1H), 3.24- 3.04 (m, 1H), 2.89- 2.55 (m, 3H), 2.46-1.63 (m, 16H), 1.61-1.39 (m, 3H), 1.20- 0.90 (m, 3H), 0.86- 0.72 (m, 3H), 0.58-0.39 (m, 2H); 950.9 [M + H]⁺ . |
| | C10 | 1H NMR (400 MHz, DMSO-d6) δ (ppm) 8.79 (d, J = 7.6 Hz, 1H), 8.27 (s, 1H), 8.11-8.00 (m, 2H), 7.60 (d, J = 8.5 Hz, 1H), 7.41- 7.26 (m, 6H), 7.24-7.13 (m, 4H), 6.28-6.09 (m, 2H), 4.71 (t, J = 8.6 Hz, 1H), 4.48 (t, J = 8.5 Hz, 1H), 4.34-4.23 (m, 1H), 4.12 (t, J = 8.6 Hz, 1H), 3.99- 3.88 (m, 2H), 3.82- 3.69 (m, 2H), 3.59-3.48 (m, 1H), 3.08-2.89 (m, 2H), 2.86- 2.65 (m, 1H), 2.40- 2.17 (m, 3H), 2.15-2.03 (m, 3H), 2.02-1.90 (m, 6H), 1.84- 1.44 (m, 11H), 1.13 (d, J = 7.1 Hz, 3H), 0.83 (t, J = 7.4 Hz, 3H), 0.53-0.36 (m, 2H); 994.6 [M + H]⁺ . |
| | C11 | 1H NMR (400 MHz, DMSO-d6) δ (ppm) 8.77 (d, J = 7.7 Hz, 1H), 8.27 (s, 1H), 8.11-8.02 (m, 2H), 7.60 (d, J = 8.5 Hz, 1H), 7.39- 7.26 (m, 6H), 7.24-7.14 (m, 4H), 6.27-5.91 (m, 3H), 4.84- 4.71 (m, 1H), 4.53- 4.43 (m, 1H), 4.36-4.26 (m, 1H), 4.17-4.05 (m, 1H), 3.99- 3.87 (m, 2H), 3.83- 3.68 (m, 2H), 3.59-3.47 (m, 1H), 3.05-2.94 (m, 1H), 2.57- 2.51 (m, 1H), 2.46- 2.32 (m, 2H), 2.26-2.17 (m, 1H), 2.16-2.08 (m, 4H), 2.05- 1.45 (m, 15H), 1.14 (d, J = 7.1 Hz, 3H), 0.83 (t, J = 7.4 Hz, 3H), 0.54-0.39 (m, 2H); 950.6 [M + H]⁺ . |
| | C12 | 1H NMR (400 MHz, DMSO-d6) δ (ppm) 8.81-8.70 (m, 1H), 8.27 (s, 1H), 8.10-8.02 (m, 2H), 7.61 (d, J = 8.5 Hz, 1H), 7.39- 7.26 (m, 6H), 7.24-7.12 (m, 4H), 6.26-5.91 (m, 3H), 4.82- 4.72 (m, 1H), 4.53- 4.43 (m, 1H), 4.35-4.26 (m, 1H), 4.15-4.07 (m, 1H), 3.91- 3.69 (m, 4H), 3.59- 3.48 (m, 1H), 3.04-2.93 (m, 1H), 2.57-2.51 (m, 1H), 2.46- 2.31 (m, 2H), 2.26- 2.17 (m, 1H), 2.16-2.08 (m, 4H), 2.07-1.89 (m, 5H), 1.89- 1.62 (m, 6H), 1.61- 1.50 (m, 2H), 1.47-1.36 (m, 2H), 1.19-1.08 (m, 3H), 0.82- 0.74 (m, 3H), 0.54- 0.37 (m, 2H); 950.4 [M + H]⁺ . |
| | C13 | 1H NMR (400 MHz, DMSO-d6) δ (ppm) 8.90-8.69 (m, 1H), 8.27 (s, 1H), 8.10-8.02 (m, 1H), 7.60 (d, J = 8.7 Hz, 1H), 7.38- 7.28 (m, 6H), 7.23-7.11 (m, 4H), 6.29-6.07 (m, 2H), 4.79- 4.63 (m, 1H), 4.54- 4.44 (m, 1H), 4.37-4.23 (m, 1H), 4.17-4.07 (m, 1H), 3.98- 3.89 (m, 1H), 3.86- 3.71 (m, 3H), 3.59-3.47 (m, 1H), 3.12-2.88 (m, 2H), 2.87- 2.71 (m, 1H), 2.38- 2.21 (m, 3H), 2.17-2.05 (m, 3H), 2.03-1.88 (m, 6H), 1.85- 1.39 (m, 11H), 1.13 (d, J = 7.0 Hz, 3H), 0.84-0.75 (m, 3H), 0.55-0.37 (m, 2H); 994.9 [M + H]⁺ . |
| | C14 | 1H NMR (400 MHz, DMSO-d6) δ (ppm) 8.79 (d, J = 7.6 Hz, 1H), 8.28 (s, 1H), 8.11-8.03 (m, 2H), 7.61 (d, J = 8.6 Hz, 1H), 7.40- 7.27 (m, 6H), 7.24-7.15 (m, 4H), 6.23-6.06 (m, 2H), 4.91- 4.66 (m, 3H), 4.52- 4.43 (m, 1H), 4.33-4.24 (m, 1H), 4.16-4.07 (m, 1H), 3.78- 3.49 (m, 3H), 3.04- 2.94 (m, 1H), 2.62-2.56 (m, 1H), 2.44-2.30 (m, 2H), 2.27- 2.12 (m, 5H), 2.02- 1.93 (m, 6H), 1.91-1.51 (m, 14H), 0.92 (d, J = 7.1 Hz, 3H), 0.55- 0.41 (m, 2H); 956.4 [M + H]⁺ . |
| | C15 | 1H NMR (400 MHz, DMSO-d6) δ (ppm) 8.91-8.76 (m, 1H), 8.33- 8.25 (m, 1H), 8.12- 8.02 (m, 2H), 7.66-7.54 (m, 1H), 7.40-7.28 (m, 6H), 7.25- 7.11 (m, 4H), 6.34- 6.12 (m, 2H), 4.93-4.65 (m, 3H), 4.55-4.43 (m, 1H), 4.35- 4.24 (m, 1H), 4.20- 4.07 (m, 1H), 3.91-3.70 (m, 2H), 3.61-3.49 (m, 1H), 3.08- 2.92 (m, 1H), 2.70- 2.57 (m, 1H), 2.46-2.31 (m, 2H), 2.30-2.08 (m, 5H), 2.04- 1.49 (m, 20H), 1.22- 1.08 (m, 3H), 0.60-0.36 (m, 2H); 956.5 [M + H]⁺ . |
| | C16 | 1H NMR (400 MHz, DMSO-d6) δ (ppm) 8.85-8.75 (m, 1H), 8.27 (s, 1H), 8.10-8.02 (m, 2H), 7.64- 7.57 (m, 1H), 7.38- 7.28 (m, 6H), 7.23-7.14 (m, 4H), 6.29-6.18 (m, 1H), 6.15- 6.06 (m, 1H), 5.20- 4.98 (m, 1H), 4.79-4.62 (m, 1H), 4.53-4.42 (m, 1H), 4.37- 4.23 (m, 1H), 4.16- 4.07 (m, 1H), 3.98-3.89 (m, 1H), 3.84-3.68 (m, 3H), 3.57- 3.49 (m, 1H), 3.14- 2.94 (m, 1H), 2.40-2.29 (m, 2H), 2.27-2.19 (m, 2H), 2.15- 2.08 (m, 2H), 2.07- 1.87 (m, 7H), 1.87-1.63 (m, 5H), 1.62-1.45 (m, 5H), 1.44- 1.37 (m, 1H), 1.16- 1.08 (m, 3H), 0.85-0.76 (m, 3H), 0.54-0.41 (m, 2H); 944.5 [M + H]⁺ . |
| | C17 | 1H NMR (400 MHz, DMSO-d6) δ (ppm) 8.80 (d, J = 7.5 Hz, 1H), 8.29 (s, 1H), 8.14-8.02 (m, 2H), 7.62 (d, J = 8.4 Hz, 1H), 7.41- 7.27 (m, 6H), 7.25-7.13 (m, 4H), 6.24-6.07 (m, 2H), 4.89- 4.68 (m, 2H), 4.48 (t, J = 8.4 Hz, 1H), 4.33-4.24 (m, 1H), 4.12 (t, J = 8.5 Hz, 1H), 3.97- 3.86 (m, 2H), 3.78- 3.65 (m, 2H), 3.59-3.49 (m, 1H), 3.06-2.92 (m, 1H), 2.64- 2.56 (m, 1H), 2.44- 2.31 (m, 2H), 2.24-1.46 (m, 21H), 0.95 (d, J = 7.1 Hz, 3H), 0.81 (t, J = 7.3 Hz, 3H), 0.56-0.36 (m, 2H); 944.8 [M + H]⁺ . |
| | C18 | 1H NMR (400 MHz, DMSO-d6) δ (ppm) 8.81-8.73 (m, 1H), 8.27 (s, 1H), 8.09-8.04 (m, 2H), 7.64- 7.57 (m, 1H), 7.38- 7.29 (m, 6H), 7.22-7.12 (m, 4H), 6.27-6.17 (m, 1H), 6.16- 6.04 (m, 1H), 4.85- 4.69 (m, 2H), 4.51-4.44 (m, 2H), 4.39-4.25 (m, 2H), 4.15- 4.08 (m, 1H), 3.97- 3.89 (m, 1H), 3.86-3.70 (m, 3H), 3.59-3.47 (m, 1H), 2.95- 2.84 (m, 1H), 2.68- 2.56 (m, 2H), 2.34-2.12 (m, 7H), 2.03-1.93 (m, 3H), 1.91- 1.63 (m, 7H), 1.57- 1.39 (m, 4H), 1.13 (d, J = 7.0 Hz, 3H), 0.86-0.76 (m, 3H), 0.53- 0.41 (m, 2H); 943.0 [M + H]⁺ . |
| | C19 | 1H NMR (400 MHz, DMSO-d6) δ (ppm) 8.79 (d, J = 7.8 Hz, 1H), 8.27 (s, 1H), 8.08-8.03 (m, 2H), 7.60 (d, J = 8.6 Hz, 1H), 7.38- 7.29 (m, 6H), 7.23-7.13 (m, 4H), 6.25-6.04 (m, 2H), 4.90- 4.68 (m, 2H), 4.47 (t, J = 8.8 Hz, 1H), 4.36-4.24 (m, 1H), 4.12 (t, J = 8.5 Hz, 1H), 3.86- 3.68 (m, 4H), 3.60- 3.49 (m, 1H), 3.04-2.96 (m, 1H), 2.64-2.55 (m, 1H), 2.43- 2.30 (m, 2H), 2.29- 2.17 (m, 1H), 2.15-1.76 (m, 12H), 1.73-1.50 (m, 6H), 1.46- 1.34 (m, 2H), 1.13 (d, J = 7.1 Hz, 3H), 0.77 (t, J = 7.4 Hz, 3H), 0.53-0.42 (m, 2H); 944.5 [M + H]⁺ . |
| | C20 | 1H NMR (400 MHz, DMSO-d6) δ (ppm) 8.79-8.71 (m, 1H), 8.27 (s, 1H), 8.10-8.03 (m, 2H), 7.63- 7.57 (m, 1H), 7.40- 7.25 (m, 6H), 7.24-7.12 (m, 4H), 6.28-6.06 (m, 2H), 4.84- 4.70 (m, 2H), 4.52- 4.42 (m, 2H), 4.40-4.34 (m, 1H), 4.33-4.25 (m, 1H), 4.12 (t, J = 8.6 Hz, 1H), 3.98- 3.89 (m, 1H), 3.89-3.68 (m, 3H), 3.59-3.48 (m, 1H), 2.95- 2.84 (m, 1H), 2.69- 2.52 (m, 3H), 2.40-2.19 (m, 3H), 2.18-2.08 (m, 4H), 2.04- 1.93 (m, 3H), 1.91- 1.62 (m, 6H), 1.61-1.37 (m, 4H), 1.17-1.08 (m, 3H), 0.85- 0.75 (m, 3H), 0.55- 0.41 (m, 2H); 943.0 [M + H]⁺ . |
| | C21 | 1H NMR (400 MHz, DMSO-d6) δ (ppm) 8.78-8.71 (m, 1H), 8.31-8.24 (m, 1H), 8.11- 8.03 (m, 2H), 7.65- 7.56 (m, 1H), 7.38- 7.27 (m, 6H), 7.22-7.10 (m, 4H), 6.29-6.10 (m, 2H), 4.84- 4.72 (m, 2H), 4.52- 4.43 (m, 2H), 4.41-4.34 (m, 1H), 4.30 (s, 1H), 4.11 (t, J = 8.5 Hz, 1H), 3.94-3.85 (m, 2H), 3.77- 3.63 (m, 2H), 3.59- 3.47 (m, 1H), 2.90 (s, 1H), 2.71- 2.58 (m, 2H), 2.40- 2.18 (m, 4H), 2.16 (s, 3H), 2.03- 1.92 (m, 3H), 1.90- 1.61 (m, 7H), 1.58-1.43 (m, 4H), 1.17 (d, J = 7.1 Hz, 1H), 0.94 (d, J = 7.1 Hz, 2H), 0.84-0.78 (m, 3H), 0.55-0.41 (m, 2H); 942.5 [M+ H]+ |
| | C22 | 1H NMR (400 MHz, DMSO-d6) δ (ppm) 8.82-8.73 (m, 1H), 8.31- 8.24 (m, 1H), 8.12- 8.02 (m, 2H), 7.64-7.56 (m, 1H), 7.40-7.27 (m, 6H), 7.24- 7.11 (m, 4H), 6.32- 6.07 (m, 2H), 4.78-4.67 (m, 1H), 4.47 (t, J = 8.6 Hz, 1H), 4.33- 4.23 (m, 1H), 4.11 (t, J = 8.6 Hz, 1H), 3.95-3.86 (m, 2H), 3.85-3.59 (m, 5H), 3.58- 3.47 (m, 2H), 3.18- 3.06 (m, 1H), 3.00-2.88 (m, 1H), 2.41-2.30 (m, 1H), 2.22 (t, J = 11.5 Hz, 1H), 2.17-2.09 (m, 2H), 2.07 (s, 3H), 2.03-1.88 (m, 4H), 1.87-1.53 (m, 8H), 1.52- 1.42 (m, 2H), 1.20- 0.92 (m, 3H), 0.85-0.76 m, 3H), 0.54-0.41 (m, 2H); 942.8 [M + H]⁺ . |
| | C23 | 1H NMR (400 MHz, DMSO-d6) δ (ppm) 8.81 (d, J = 7.6 Hz, 1H), 8.28 (s, 1H), 8.11-8.03 (m, 2H), 7.60 (d, J = 8.4 Hz, 1H), 7.39- 7.15 (m, 10H), 6.28-6.11 (m, 2H), 4.90-4.67 (m, 2H), 4.48 (t, J = 8.4 Hz, 1H), 4.34- 4.24 (m, 1H), 4.16-4.07 (m, 1H), 3.99-3.88 (m, 2H), 3.83- 3.70 (m, 2H), 3.60- 3.48 (m, 1H), 3.05-2.95 (m, 1H), 2.65-2.55 (m, 1H), 2.45- 2.29 (m, 2H), 2.23 (t, J = 12.0 Hz, 1H), 2.18-1.92 (m, 8H), 1.91- 1.62 (m, 6H), 1.62- 1.44 (m, 6H), 1.14 (d, J = 7.2 Hz, 3H), 0.83 (t, J = 7.4 Hz, 3H), 0.55- 0.41 (m, 2H); 944.4 [M + H]⁺ . |
| | C24 | 1H NMR (400 MHz, DMSO-d6) δ (ppm) 8.83-8.73 (m, 1H), 8.27 (s, 1H), 8.09-8.02 (m, 2H), 7.64- 7.56 (m, 1H), 7.38- 7.26 (m, 6H), 7.24-7.13 (m, 4H), 6.28-6.05 (m, 2H), 4.77- 4.66 (m, 1H), 4.47 (t, J = 8.6 Hz, 1H), 4.34-4.23 (m, 1H), 4.12 (t, J = 8.5 Hz, 1H), 3.98- 3.89 (m, 1H), 3.86- 3.60 (m, 6H), 3.59-3.47 (m, 2H), 3.17-3.06 (m, 1H), 2.99- 2.87 (m, 1H), 2.40- 2.30 (m, 1H), 2.23 (t, J = 11.5 Hz, 1H), 2.16- 2.09 (m, 2H), 2.07 (s, 3H), 2.03-1.87 (m, 4H), 1.85- 1.48 (m, 9H), 1.47- 1.36 (m, 1H), 1.17-1.09 (m, 3H), 0.85-0.75 (m, 3H), 0.54- 0.41 (m, 2H); 942.6 [M + H]⁺ . |
| | C25 | 1H NMR (400 MHz, DMSO-d6) δ (ppm) 10.05-9.43 (m, 1H), 8.94- 8.75 (m, 1H), 8.32- 8.21 (m, 1H), 8.11-7.98 (m, 2H), 7.60 (d, J = 8.7 Hz, 1H), 7.39- 7.25 (m, 6H), 7.24- 7.09 (m, 4H), 6.32-6.14 (m, 2H), 5.04-4.75 (m, 2H), 4.64- 4.44 (m, 2H), 4.22- 4.06 (m, 1H), 3.97-3.82 (m, 3H), 3.79-3.69 (m, 2H), 3.56- 3.53 (m, 2H), 3.36- 2.99 (m, 1H), 2.84-2.62 (m, 2H), 2.61-2.55 (m, 3H), 2.48- 2.31 (m, 2H), 2.26- 1.43 (m, 15H), 1.17 (d, J = 7.0 Hz, 3H), 0.81 (t, J = 7.4 Hz, 3H), 0.63- 0.44 (m, 2H); 944.6 [M + H]⁺ . |
| | C26 | 1H NMR (400 MHz, DMSO-d6) δ (ppm) 9.12-8.72 (m, 1H), 8.37- 8.21 (m, 1H), 8.16- 8.00 (m, 2H), 7.62 (d, J = 8.6 Hz, 1H), 7.44-7.10 (m, 10H), 6.30- 6.05 (m, 2H), 4.89- 4.77 (m, 1H), 4.67-4.37 (m, 3H), 4.31-4.01 (m, 4H), 3.98- 3.78 (m, 6H), 3.58- 3.51 (m, 3H), 3.11-2.68 (m, 4H), 2.41-1.70 (m, 12H), 1.66- 1.34 (m, 3H), 1.14 (d, J = 7.1 Hz, 3H), 0.90-0.71 (m, 3H), 0.62-0.39 (m, 2H); 942.6 [M + H]⁺ . |
| | C27 | 1H NMR (400 MHz, DMSO-d6) δ (ppm) 8.86-8.65 (m, 1H), 8.36- 8.22 (m, 1H), 8.14- 8.00 (m, 2H), 7.60 (t, J = 7.7 Hz, 1H), 7.45-7.25 (m, 6H), 7.25- 7.07 (m, 4H), 6.37- 5.96 (m, 2H), 4.81-4.61 (m, 1H), 4.47 (t, J = 8.5 Hz, 1H), 4.37- 4.22 (m, 1H), 4.12 (t, J = 8.4 Hz, 1H), 4.01-3.85 (m, 3H), 3.78-3.64 (m, 4H), 3.59- 3.46 (m, 1H), 3.24- 2.92 (m, 2H), 2.44- 1.33 (m, 22H), 1.19-0.91 (m, 3H), 0.81 (t, J = 7.4 Hz, 3H), 0.58- 0.36 (m, 2H); 942.6 [M + H]⁺ . |
| | C28 | 1H NMR (400 MHz, DMSO-d6) δ (ppm) 8.87-8.74 (m, 1H), 8.61- 8.53 (m, 1H), 8.47- 8.38 (m, 1H), 8.29-8.24 (m, 1H), 8.11-8.01 (m, 2H), 7.81- 7.73 (m, 1H), 7.63- 7.55 (m, 1H), 7.39-7.27 (m, 3H), 7.22-7.11 (m, 3H), 6.30- 6.05 (m, 2H), 4.90- 4.65 (m, 2H), 4.54-4.43 (m, 1H), 4.36-4.24 (m, 1H), 4.19- 4.08 (m, 1H), 3.97- 3.87 (m, 1H), 3.86-3.69 (m, 3H), 3.64-3.49 (m, 1H), 3.13- 2.92 (m, 1H), 2.65- 2.56 (m, 1H), 2.45-2.30 (m, 2H), 2.23-1.35 (m, 21H), 1.18- 1.08 (m, 3H), 0.89- 0.71 (m, 3H), 0.54-0.37 (m, 2H); 945.6 [M + H]⁺ . |
| | C29 | 1H NMR (400 MHz, DMSO-d6) δ (ppm) 8.75 (d, J = 7.1 Hz, 1H), 8.58 (s, 1H), 8.43 (d, J = 4.0 Hz, 1H), 8.28 (s, 1H), 8.13-8.02 (m, 2H), 7.78 (d, J = 7.6 Hz, 1H), 7.60 (d, J = 8.4 Hz, 1H), 7.43-7.27 (m, 3H), 7.19 (d, J = 7.6 Hz, 3H), 6.31- 6.10 (m, 2H), 5.01- 4.90 (m, 1H), 4.55-4.47 (m, 1H), 4.36-4.27 (m, 1H), 4.17- 4.09 (m, 1H), 3.97- 3.89 (m, 2H), 3.83-3.73 (m, 2H), 3.65-3.53 (m, 5H), 2.86- 2.76 (m, 1H), 2.46- 2.31 (m, 4H), 2.25-1.57 (m, 14H), 1.56-1.45 (m, 2H), 1.14 (d, J = 6.9 Hz, 3H), 0.83 (t, J = 7.3 Hz, 3H), 0.56-0.35 (m, 2H); 929.2 [M + H]⁺ . |
| | C30 | 1H NMR (400 MHz, DMSO-d6) δ (ppm) 8.78 (d, J = 7.6 Hz, 1H), 8.26 (s, 1H), 8.11-7.96 (m, 2H), 7.63- 7.56 (m, 1H), 7.40- 7.26 (m, 6H), 7.25-7.08 (m, 4H), 6.31-6.13 (m, 2H), 4.77- 4.66 (m, 1H), 4.58- 4.50 (m, 2H), 4.49-4.38 (m, 2H), 4.38-4.25 (m, 2H), 4.11 (t, J = 8.6 Hz, 1H), 3.94- 3.83 (m, 3H), 3.78-3.69 (m, 2H), 3.60-3.48 (m, 1H), 2.87- 2.78 (m, 1H), 2.42- 2.30 (m, 1H), 2.22 (t, J = 11.5 Hz, 1H), 2.16- 2.07 (m, 1H), 2.05- 1.91 (m, 6H), 1.86-1.44 (m, 10H), 1.17 (d, J = 7.1 Hz, 3H), 0.85- 0.75 (m, 3H), 0.56- 0.42 (m, 2H); 928.5 [M + H]⁺ . |
| | C31 | 1H NMR (400 MHz, DMSO-d6) δ (ppm) 8.85-8.69 (m, 1H), 8.42 (s, 1H), 8.29 (s, 1H), 8.24-8.18 (m, 1H), 8.10-8.04 (m, 2H), 7.63- 7.56 (m, 1H), 7.40- 7.32 (m, 2H), 7.23-7.14 (m, 3H), 6.96-6.89 (m, 1H), 6.29- 6.08 (m, 2H), 5.03- 4.93 (m, 1H), 4.52-4.43 (m, 1H), 4.36-4.23 (m, 1H), 4.18- 4.08 (m, 1H), 4.00- 3.87 (m, 2H), 3.86-3.68 (m, 3H), 3.59 (s, 4H), 2.74 (s, 7H), 2.48- 2.22 (m, 5H), 2.19- 2.09 (m, 2H), 2.07-1.60 (m, 11H), 1.56-1.43 (m, 2H), 1.18- 1.07 (m, 3H), 0.88- 0.78 (m, 3H), 0.55-0.43(m, 2H); 972.5 [M + H]⁺ . |
| | C32 | 1H NMR (400 MHz, DMSO-d6) δ (ppm) 8.86-8.66 (m, 1H), 8.26 (s, 1H), 8.10-8.00 (m, 2H), 7.64- 7.56 (m, 1H), 7.41- 7.11 (m, 10H), 6.29-6.02 (m, 2H), 4.79-4.64 (m, 1H), 4.52- 4.40 (m, 1H), 4.33- 4.21 (m, 1H), 4.15-4.04 (m, 1H), 3.97-3.49 (m, 8H), 3.15- 2.97 (m, 2H), 2.38- 1.36 (m, 22H), 1.19-1.05 (m, 3H), 0.88-0.73 (m, 3H), 0.56- 0.36 (m, 2H); 942.5 [M + H]⁺ . |
| | C33 | 1H NMR (400 MHz, DMSO-d6) δ (ppm) 8.78-8.69 (m, 1H), 8.60- 8.55 (m, 1H), 8.46- 8.37 (m, 1H), 8.31-8.21 (m, 1H), 8.09-8.00 (m, 2H), 7.83- 7.72 (m, 1H), 7.65- 7.53 (m, 1H), 7.39-7.28 (m, 3H), 7.20-7.09 (m, 3H), 6.32- 6.13 (m, 2H), 5.06- 4.84 (m, 1H), 4.57-4.43 (m, 1H), 4.38-4.24 (m, 1H), 4.19- 4.06 (m, 1H), 3.96- 3.75 (m, 4H), 3.63-3.52 (m, 5H), 2.82-2.64 (m, 1H), 2.44- 2.29 (m, 4H), 2.23- 1.58 (m, 14H), 1.51-1.41 (m, 2H), 1.20-1.12 (m, 3H), 0.85- 0.76 (m, 3H), 0.55- 0.38 (m, 2H); 929.4 [M + H]⁺ . |
| | C34 | 1H NMR (400 MHz, DMSO-d6) δ (ppm) 8.79 (d, J = 7.7 Hz, 1H), 8.27 (s, 1H), 8.10-8.03 (m, 2H), 7.60 (d, J = 8.6 Hz, 1H), 7.39- 7.27 (m, 6H), 7.24-7.14 (m, 4H), 6.28-6.11 (m, 2H), 4.78- 4.64 (m, 1H), 4.59- 4.50 (m, 2H), 4.50-4.38 (m, 2H), 4.38-4.33 (m, 1H), 4.33- 4.25 (m, 1H), 4.16- 4.06 (m, 1H), 3.99-3.88 (m, 2H), 3.83-3.65 (m, 3H), 3.60- 3.48 (m, 1H), 2.90- 2.76 (m, 1H), 2.42-2.30 (m, 1H), 2.28-2.18 (m, 1H), 2.15- 2.07 (m, 1H), 2.05- 1.89 (m, 6H), 1.87-1.56 (m, 7H), 1.55-1.44 (m, 3H), 1.13 (d, J = 7.1 Hz, 3H), 0.83 (t, J = 7.2 Hz, 3H), 0.59-0.40 (m, 2H); 928.4 [M + H]⁺ . |
| | C35 | 1H NMR (400 MHz, DMSO-d6) δ (ppm) 8.77 (d, J = 7.7 Hz, 1H), 8.27 (s, 1H), 8.11-8.03 (m, 2H), 7.61 (d, J = 8.5 Hz, 1H), 7.39- 7.27 (m, 6H), 7.24-7.15 (m, 4H), 6.23-6.07 (m, 2H), 4.74- 4.64 (m, 1H), 4.57- 4.50 (m, 2H), 4.50-4.38 (m, 2H), 4.37-4.26 (m, 2H), 4.16- 4.07 (m, 1H), 3.97- 3.86 (m, 2H), 3.79-3.64 (m, 3H), 3.60-3.48 (m, 1H), 2.88- 2.79 (m, 1H), 2.40- 2.29 (m, 1H), 2.27-2.17 (m, 1H), 2.14-2.07 (m, 1H), 2.03- 1.91 (m, 6H), 1.85- 1.46 (m, 10H), 0.94 (d, J = 7.1 Hz, 3H), 0.81 (t, J = 7.4 Hz, 3H), 0.56- 0.42 (m, 2H); 929.0 [M + H]⁺ . |
| | C36 | 1H NMR (400 MHz, DMSO-d6) δ (ppm) 8.83-8.72 (m, 1H), 8.30- 8.22 (m, 1H), 8.10- 8.02 (m, 2H), 7.63-7.56 (m, 1H), 7.38-7.31 (m, 3H), 7.23- 7.12 (m, 5H), 7.07- 7.00 (m, 1H), 6.29-6.17 (m, 1H), 6.14-6.06 (m, 1H), 4.76- 4.66 (m, 1H), 4.57- 4.27 (m, 6H), 4.18-4.08 (m, 1H), 3.99-3.87 (m, 1H), 3.86- 3.69 (m, 4H), 3.62- 3.52 (m, 1H), 2.90-2.75 (m, 1H), 2.41-2.32 (m, 1H), 2.25- 2.17 (m, 1H), 2.12- 1.91 (m, 7H), 1.84-1.38 (m, 10H), 1.13 (d, J = 7.1 Hz, 3H), 0.85- 0.75 (m, 3H), 0.57- 0.40 (m, 2H); 946.6 [M + H]⁺ . |
| | C37 | 1H NMR (400 MHz, DMSO-d6) δ (ppm) 8.81-8.74 (m, 1H), 8.27 (s, 1H), 8.09-8.02 (m, 2H), 7.64- 7.58 (m, 1H), 7.40- 7.12 (m, 10H), 6.27-6.03 (m, 2H), 4.80-4.63 (m, 1H), 4.59- 4.23 (m, 6H), 4.18- 4.04 (m, 1H), 3.89-3.66 (m, 5H), 3.59-3.48 (m, 1H), 2.88- 2.76 (m, 1H), 2.41- 2.29 (m, 1H), 2.27-2.16 (m, 1H), 2.15-2.07 (m, 1H), 2.06- 1.86 (m, 6H), 1.85- 1.34 (m, 10H), 1.17-1.08 (m, 3H), 0.82-0.73 (m, 3H), 0.57- 0.42 (m, 2H); 928.5 [M + H]⁺ . |
| | C38 | 1H NMR (400 MHz, MeOD) 8 8.13-8.02 (m, 2H), 7.97-7.94 (m, 1H), 7.66- 7.61 (m, 1H), 7.43- 7.27 (m, 7H), 7.25-7.12 (m, 3H), 6.19-5.95 (m, 1H), 5.04- 4.93 (m, 1H), 4.66- 4.62 (m, 1H), 4.55-4.38 (m, 2H), 4.00-3.78 (m, 4H), 3.75- 3.69 (m, 5H), 2.95- 2.85 (m, 1H), 2.67-2.49 (m, 4H), 2.40-2.25 (m, 2H), 2.21- 1.93 (m, 5H), 1.90- 1.82 (m, 3H), 1.78-1.65 (m, 1H), 1.62-1.46 (m, 2H), 1.37- 1.26 (m, 2H), 1.23- 1.21 (m, 3H), 1.00-0.92 (m, 1H), 0.90-0.84 (m, 3H), 0.76- 0.65 (m, 1H); 953.2 [M + H]⁺ . |
| | C39 | 1H NMR (400 MHz, DMSO-d6) δ (ppm) 8.84-8.72 (m, 1H), 8.62- 8.55 (m, 1H), 8.47- 8.40 (m, 1H), 8.31-8.24 (m, 1H), 8.11-8.03 (m, 2H), 7.82- 7.75 (m, 1H), 7.66- 7.56 (m, 1H), 7.39-7.30 (m, 3H), 7.22-7.13 (m, 3H), 6.29- 6.05 (m, 2H), 4.97- 4.80 (m, 1H), 4.57-4.49 (m, 1H), 4.18-4.10 (m, 1H), 3.97- 3.54 (m, 9H), 3.36- 3.29 (m, 5H), 2.26-1.34 (m, 16H), 1.13 (d, J = 7.1 Hz, 3H), 0.87- 0.73 (m, 3H), 0.57- 0.40 (m, 2H); 929.7 [M+ H]+ |
| | C40 | 1H NMR (400 MHz, DMSO-d6) δ (ppm) 8.74 (d, J = 7.5 Hz, 1H), 8.58 (d, J = 2.0 Hz, 1H), 8.43 (dd, J = 4.7, 1.5 Hz, 1H), 8.28 (s, 1H), 8.12-7.97 (m, 2H), 7.78 (d, J = 8.0 Hz, 1H), 7.61 (d, J = 8.7 Hz, 1H), 7.42-7.27 (m, 3H), 7.24-7.09 (m, 3H), 6.29 6.04 (m, 2H), 5.03- 4.88 (m, 1H), 4.58-4.43 (m, 1H), 4.40-4.25 (m, 1H), 4.21- 4.07 (m, 1H), 3.89- 3.72 (m, 4H), 3.63-3.51 (m, 7H), 2.82-2.69 (m, 1H), 2.46- 2.29 (m, 4H), 2.12- 1.97 (m, 4H), 1.81-1.59 (m, 8H), 1.45-1.36 (m, 2H), 1.13 (d, J = 7.1 Hz, 3H), 0.78 (t, J = 7.4 Hz, 3H), 0.61-0.33 (m, 2H); 930.0 [M + H]⁺ . |
| | C41 | 1H NMR (400 MHz, DMSO-d6) δ (ppm) 8.79-8.66 (m, 1H), 8.61- 8.51 (m, 1H), 8.49- 8.40 (m, 1H), 8.29 (s, 1H), 8.14- 7.99 (m, 2H), 7.82- 7.73 (m, 1H), 7.66-7.55 (m, 1H), 7.42-7.27 (m, 3H), 7.22- 7.06 (m, 3H), 6.32- 6.01 (m, 2H), 5.09-4.81 (m, 1H), 4.65-4.21 (m, 3H), 3.93- 3.69 (m, 3H), 3.68- 3.44 (m, 6H), 2.82-2.68 (m, 1H), 2.48-2.22 (m, 6H), 2.10- 1.55 (m, 12H), 1.47- 1.34 (m, 2H), 1.18-0.99 (m, 3H), 0.83-0.69 (m, 3H), 0.49 (s, 2H); 930.6 [M + H]⁺ . |
| | C42 | 1H NMR (400 MHz, DMSO-d6) δ (ppm) 8.80-8.71 (m, 1H), 8.61- 8.54 (m, 1H), 8.48- 8.42 (m, 1H), 8.31-8.26 (m, 1H), 8.10-8.04 (m, 2H), 7.82- 7.74 (m, 1H), 7.64- 7.57 (m, 1H), 7.41-7.31 (m, 3H), 7.22-7.14 (m, 3H), 6.29- 6.10 (m, 2H), 5.04- 4.89 (m, 1H), 4.60-4.49 (m, 1H), 4.48-4.36 (m, 1H), 4.35- 4.22 (m, 1H), 4.01- 3.86 (m, 2H), 3.84-3.71 (m, 1H), 3.66-3.49 (m, 6H), 2.84- 2.70 (m, 1H), 2.49- 2.42 (m, 2H), 2.42-2.23 (m, 4H), 2.12-1.45 (m, 15H), 1.19- 1.08 (m, 3H), 0.89- 0.76 (m, 3H), 0.55-0.41 (m, 2H); 929.6 [M + H]⁺ . |
| | C43 | 1H NMR (400 MHz, DMSO-d6) δ (ppm) 8.79-8.65 (m, 1H), 8.63 8.51 (m, 1H), 8.46- 8.38 (m, 1H), 8.28 (s, 1H), 8.14- 7.99 (m, 2H), 7.82- 7.73 (m, 1H), 7.65-7.56 (m, 1H), 7.41-7.29 (m, 3H), 7.24 7.14 (m, 3H), 6.25- 6.03 (m, 2H), 5.04-4.87 (m, 1H), 4.56-4.44 (m, 1H), 4.37- 4.24 (m, 1H), 4.20- 4.06 (m, 1H), 3.98-3.85 (m, 2H), 3.84-3.49 (m, 7H), 2.80- 2.69 (m, 1H), 2.48- 1.57 (m, 18H), 1.54-1.42 (m, 2H), 0.98-0.89 (m, 3H), 0.85- 0.74 (m, 3H), 0.56- 0.37 (m, 2H); 929.8 [M + H]⁺ . |
| | C44 | 1H NMR (400 MHz, DMSO-d6) δ (ppm) 8.80-8.73 (m, 1H), 8.72- 8.67 (m, 1H), 8.56- 8.50 (m, 1H), 8.29 (s, 1H), 8.09- 8.03 (m, 2H), 7.95- 7.89 (m, 1H), 7.64-7.59 (m, 1H), 7.46-7.40 (m, 1H), 7.38- 7.33 (m, 2H), 7.19- 7.13 (m, 3H), 6.26-6.06 (m, 2H), 5.00-4.92 (m, 1H), 4.55- 4.41 (m, 2H), 4.35- 4.28 (m, 1H), 4.08-4.02 (m, 1H), 3.86-3.73 (m, 5H), 3.61- 3.55 (m, 4H), 2.77- 2.71 (m, 1H), 2.46-2.16 (m, 6H), 2.03-1.66 (m, 10H), 1.46- 1.37 (m, 2H), 1.16- 1.09 (m, 3H), 0.84-0.76 (m, 4H), 0.72-0.65 (m, 1H); 954.8 [M + H]⁺ . |
| | C45 | 1H NMR (400 MHz, DMSO-d6) δ (ppm) 8.78 (d, J = 7.6 Hz, 1H), 8.72- 8.67 (m, 1H), 8.57- 8.51 (m, 1H), 8.29 (s, 1H), 8.11- 8.04 (m, 2H), 7.95- 7.90 (m, 1H), 7.60 (d, J = 8.6 Hz, 1H), 7.46-7.40 (m, 1H), 7.39- 7.33 (m, 2H), 7.22- 7.14 (m, 3H), 6.28-6.11 (m, 2H), 5.01-4.90 (m, 1H), 4.56- 4.49 (m, 1H), 4.48- 4.40 (m, 1H), 4.36-4.26 (m, 1H), 4.06 (d, J = 9.3 Hz, 1H), 4.00- 3.88 (m, 2H), 3.85- 3.71 (m, 3H), 3.65-3.51 (m, 4H), 2.80-2.70 (m, 1H), 2.48- 2.33 (m, 4H), 2.33- 2.16 (m, 2H), 2.04-1.91 (m, 3H), 1.89-1.60 (m, 7H), 1.56- 1.46 (m, 2H), 1.14 (d, J = 7.1 Hz, 3H), 0.86-0.78 (m, 4H), 0.73-0.64 (m, 1H); 954.6 [M + H]⁺ . |
| | C46 | 1H NMR (400 MHz, DMSO-d6) δ (ppm) 8.80-8.70 (m, 1H), 8.42 (s, 1H), 8.29 (s, 1H), 8.24-8.18 (m, 1H), 8.08-8.03 (m, 2H), 7.64- 7.56 (m, 1H), 7.41- 7.32 (m, 2H), 7.21-7.11 (m, 3H), 6.97-6.90 (m, 1H), 6.30- 6.03 (m, 2H), 5.04- 4.91 (m, 1H), 4.56-4.44 (m, 1H), 4.36-4.23 (m, 1H), 4.18- 4.07 (m, 1H), 3.87- 3.69 (m, 5H), 3.59 (s, 4H), 2,84- 2.70 (m, 7H), 2.49- 2.23 (m, 5H), 2.20-1.57 (m, 13H), 1.49-1.33 (m, 2H), 1.12 (d, J = 7.1 Hz, 3H), 0.78 (t, J = 7.2 Hz, 3H), 0.57-0.43 (m, 2H); 972.6 [M + H]⁺ . |
| | C47 | 1H NMR (400 MHz, DMSO-d6) δ (ppm) 8.81-8.70 (m, 1H), 8.42 (s, 1H), 8.28 (s, 1H), 8.23-8.19 (m, 1H), 8.08-8.02 (m, 2H), 7.62- 7.56 (m, 1H), 7.38- 7.31 (m, 2H), 7.19-7.09 (m, 3H), 6.95-6.90 (m, 1H), 6.30- 6.13 (m, 2H), 5.01- 4.93 (m, 1H), 4.52-4.44 (m, 1H), 4.34-4.25 (m, 1H), 4.17- 4.08 (m, 1H), 3.95- 3.72 (m, 5H), 3.63-3.52 (m, 4H), 2.74 (s, 6H), 2.48-2.25 (m, 6H), 2.19-2.10 (m, 2H), 2.08- 1.87 (m, 5H), 1.81- 1.58 (m, 6H), 1.52-1.41 (m, 2H), 1.20-1.12 (m, 3H), 0.81 (t, J = 7.4 Hz, 3H), 0.53- 0.44 (m, 2H); 972.5 [M + H]⁺ . |
| | C48 | 1H NMR (400 MHz, DMSO-d6) δ (ppm) 8.75 (d, J = 7.2 Hz, 1H), 8.42 (s, 1H), 8.29 (s, 1H), 8.21 (d, J = 5.2 Hz, 1H), 8.12- 8.04 (m, 2H), 7.61 (d, J = 8.4 Hz, 1H), 7.42-7.31 (m, 2H), 7.27-7.14 (m, 3H), 6.93 (d, J = 5.2 Hz, 1H), 6.25- 6.03 (m, 2H), 5.04-4.90 (m, 1H), 4.53-4.44 (m, 1H), 4.36- 4.25 (m, 1H), 4.18- 4.07 (m, 1H), 3.96-3.86 (m, 2H), 3.84-3.54 (m, 7H), 2.84- 2.65 (m, 7H), 2.49- 1.43 (m, 20H), 0.94 (d, J = 6.8 Hz, 3H), 0.81 (t, J = 6.8 Hz, 3H), 0.56- 0.42 (m, 2H); 972.5 [M + H]⁺ . |
| | C49 | 1H NMR (400 MHz, DMSO-d6) δ (ppm) 8.81-8.68 (m, 1H), 8.62- 8.54 (m, 1H), 8.46- 8.36 (m, 1H), 8.28 (s, 1H), 8.12- 8.00 (m, 2H), 7.83- 7.74 (m, 1H), 7.65-7.55 (m, 1H), 7.42-7.27 (m, 3H), 7.24- 7.11 (m, 3H), 6.28- 6.08 (m, 2H), 5.04-4.91 (m, 1H), 4.90-4.74 (m, 1H), 4.58- 4.42 (m, 1H), 4.38- 4.24 (m, 1H), 4.20-4.07 (m, 1H), 3.89-3.65 (m, 2H), 3.64- 3.46 (m, 5H), 2.82- 2.61 (m, 1H), 2.46-2.43 (m, 1H), 2.42-2.30 (m, 3H), 2.25- 1.53 (m, 14H), 1.15- 1.08 (m, 9H), 0.60-0.33 (m, 2H); 929.6 [M + H]⁺ . |
| | C50 | 1H NMR (400 MHz, DMSO-d6) δ (ppm) 8.76-8.70 (m, 1H), 8.60- 8.56 (m, 1H), 8.45- 8.40 (m, 1H), 8.27 (s, 1H), 8.08- 8.03 (m, 2H), 7.81- 7.76 (m, 1H), 7.63-7.58 (m, 1H), 7.38-7.30 (m, 3H), 7.19- 7.12 (m, 3H), 6.27- 6.04 (m, 2H), 5.00-4.91 (m, 1H), 4.77-4.70 (m, 1H), 4.54 4.46 (m, 1H), 4.35- 4.26 (m, 1H), 4.18-4.11 (m, 1H), 3.83-3.73 (m, 2H), 3.62- 3.52 (m, 5H), 2.79- 2.69 (m, 1H), 2.45-2.32 (m, 4H), 2.22-1.91 (m, 7H), 1.87- 1.58 (m, 7H), 1.14- 1.07 (m, 6H), 1.02 (d, J = 6.2 Hz, 3H), 0.53-0.41 (m, 2H); 929.6 [M + H]⁺ . |
| | C51 | 1H NMR (400 MHz, DMSO-d6) δ (ppm) 8.80-8.68 (m, 1H), 8.27 (d, J = 3.6 Hz, 1H), 8.12- 7.99 (m, 2H), 7.65-7.55 (m, 1H), 7.40-7.26 (m, 6H), 7.23- 7.10 (m, 4H), 6.30- 6.07 (m, 2H), 4.85-4.67 (m, 2H), 4.53-4.42 (m, 2H), 4.41- 4.34 (m, 1H), 4.33- 4.25 (m, 1H), 4.16-4.06 (m, 1H), 3.96-3.63 (m, 4H), 3.58- 3.47 (m, 1H), 2.96- 2.84 (m, 1H), 2.73-2.52 (m, 3H), 2.41-2.21 (m, 3H), 2.20- 2.08 (m, 4H), 2.04- 1.92 (m, 3H), 1.90-1.61 (m, 6H), 1.59-1.40 (m, 4H), 1.19- 0.92 (m, 3H), 0.81 (t, J = 7.4 Hz, 3H), 0.55-0.39 (m, 2H); 942.5 [M + H]⁺ . |
| | C52 | 1H NMR (400 MHz, DMSO-d6) δ (ppm) 8.79 (M, 1H), 8.28 (s, 1H), 8.10-8.02 (m, 2H), 7.60 (t, J = 7.5 Hz, 1H), 7.39- 7.11 (m, 16H), 6.37- 6.05 (m, 3H), 5.09-4.90 (m, 2H), 4.84-4.72 (m, 1H), 4.50 (t, J = 8.5 Hz, 1H), 4.38- 4.27 (m, 1H), 4.16-4.06 (m, 1H), 3.97-3.83 (m, 1H), 3.75 (t, J = 9.7 Hz, 1H), 3.60- 3.49 (m, 1H), 3.06-2.94 (m, 1H), 2.47-2.33 (m, 2H), 2.25- 2.19 (m, 1H), 2.16- 2.11 (m, 4H), 2.07-1.93 (m, 5H), 1.89-1.54 (m, 8H), 1.22- 1.15 (m, 3H), 0.55- 0.41 (m, 2H); 998.6 [M + H]⁺ . |
| | C53 | 1H NMR (400 MHz, DMSO-d6) δ (ppm) 9.86-9.38 (m, 1H), 8.91- 8.74 (m, 1H), 8.31- 8.23 (m, 1H), 8.11-8.03 (m, 2H), 7.61 (d, J = 8.5 Hz, 1H), 7.39 7.27 (m, 6H), 7.24- 7.11 (m, 4H), 6.39-5.95 (m, 3H), 4.87-4.76 (m, 1H), 4.63- 4.47 (m, 2H), 4.17- 4.08 (m, 1H), 3.98-3.89 (m, 1H), 3.79-3.68 (m, 3H), 3.60- 3.56 (m, 2H), 3.35- 3.12 (m, 2H), 2.82-2.68 (m, 3H), 2.48-1.68 (m, 15H), 1.61- 1.35 (m, 5H), 0.84 (t, J = 7.4 Hz, 1H), 0.79-0.72 (m, 3H), 0.63 (t, J = 7.4 Hz, 2H), 0.57- 0.43 (m, 2H); 964.5 [M + H]⁺ . |
| | C54 | 1H NMR (400 MHz, DMSO-d6) δ (ppm) 8.81-8.71 (m, 1H), 8.27 (s, 1H), 8.07 (d, J = 7.5 Hz, 2H), 7.60 (d, J = 8.5 Hz, 1H), 7.38-7.28 (m, 6H), 7.23-7.12 (m, 4H), 6.29- 5.93 (m, 3H), 4.78 (d, J = 7.6 Hz, 1H), 4.53-4.43 (m, 1H), 4.31 (s, 1H), 4.15-3.96 (m, 3H), 3.92-3.69 (m, 2H), 3.57- 3.49 (m, 1H), 3.47- 3.37 (m, 2H), 3.21 (d, J = 5.6 Hz, 3H), 2.99 (s, 1H), 2.44-2.32 (m, 2H), 2.26-2.18 (m, 1H), 2.16- 1.51 (m, 18H), 1.18- 1.10 (m, 3H), 0.52-0.40 (m, 2H); 966.5 [M + H]⁺ . |
| | C55 | 1H NMR (400 MHz, DMSO-d6) δ (ppm) 8.92-8.67 (m, 1H), 8.28 (s, 1H), 8.14-7.96 (m, 2H), 7.68- 7.54 (m, 1H), 7.43- 7.27 (m, 6H), 7.25-7.10 (m, 4H), 6.32-6.06 (m, 2H), 4.90- 4.60 (m, 2H), 4.48 (t, J = 8.5 Hz, 1H), 4.36-4.22 (m, 1H), 4.12 (t, J = 8.4 Hz, 1H), 3.99- 3.69 (m, 4H), 3.63- 3.46 (m, 1H), 3.10-2.96 (m, 1H), 2.77-2.62 (m, 1H), 2.60- 2.53 (m, 1H), 2.47- 1.32 (m, 21H), 1.13 (d, J = 7.0 Hz, 3H), 0.98 (t, J = 6.9 Hz, 3H), 0.88- 0.72 (m, 3H), 0.60- 0.35 (m, 2H); 958.5 [M + H]⁺ . |
| | C56 | 1H NMR (400 MHz, DMSO-d6) δ (ppm) 8.84-8.73 (m, 1H), 8.27 (d, J = 3.7 Hz, 1H), 8.11- 8.01 (m, 2H), 7.60 (t, J = 7.6 Hz, 1H), 7.41-7.28 (m, 6H), 7.23- 7.11 (m, 4H), 6.29- 6.08 (m, 2H), 4.88-4.66 (m, 2H), 4.47 (t, J = 8.5 Hz, 1H), 4.33- 4.23 (m, 1H), 4.12 (t, J = 8.5 Hz, 1H), 3.94-3.69 (m, 4H), 3.60-3.48 (m, 1H), 3.09- 2.98 (m, 1H), 2.74- 2.66 (m, 1H), 2.61-2.53 (m, 1H), 2.47-1.92 (m, 11H), 1.89- 1.62 (m, 6H), 1.60- 1.43 (m, 4H), 1.17 (d, J = 7.1 Hz, 1H), 1.03-0.90 (m, 5H), 0.81 (t, J = 7.4 Hz, 3H), 0.55- 0.41 (m, 2H); 958.6 [M + H]⁺ . |
| | C57 | 1H NMR (400 MHz, DMSO-d6) δ (ppm) 8.84-8.70 (m, 1H), 8.27 (s, 1H), 8.11-7.98 (m, 2H), 7.61 (d, J = 8.4 Hz, 1H), 7.44- 7.06 (m, 10H), 6.31-5.89 (m, 3H), 4.91-4.64 (m, 2H), 4.56- 4.41 (m, 1H), 4.38- 4.25 (m, 1H), 4.14-4.01 (m, 1H), 3.84-3.64 (m, 2H), 3.60- 3.43 (m, 1H), 2.99 (s, 1H), 2.44-2.27 (m, 2H), 2.24- 1.49 (m, 18H), 1.20- 0.94 (m, 9H), 0.55-0.35 (m, 2H); 950.6 [M + H]⁺ . |
| | C58 | 1H NMR (400 MHz, DMSO-d6) δ 8.81-8.72 (m, 1H), 8.27 (s, 1H), 8.11-8.03 (m, 2H), 7.66-7.57 (m, 1H), 7.38- 7.28 (m, 6H), 7.23- 7.13 (m, 4H), 6.26-5.93 (m, 3H), 4.83-4.71 (m, 1H), 4.54- 4.43 (m, 1H), 4.38- 4.26 (m, 1H), 4.16-4.07 (m, 1H), 4.06-3.97 (m, 1H), 3.95- 3.69 (m, 3H), 3.59- 3.48 (m, 1H), 3.09-2.89 (m, 1H), 2.44-2.32 (m, 2H), 2.25- 1.53 (m, 19H), 1.15- 1.01 (m, 6H), 0.54-0.40 (m, 2H); 936.7 [M + H]⁺ . |
| | C59 | 1H NMR (400 MHz, DMSO-d6) δ (ppm) 8.78 (d, J = 7.7 Hz, 1H), 8.27 (s, 1H), 8.11-8.01 (m, 2H), 7.61 (d, J = 8.6 Hz, 1H), 7.39- 7.13 (m, 10H), 6.27-6.04 (m, 2H), 4.79-4.66 (m, 1H), 4.52- 4.39 (m, 1H), 4.34- 4.21 (m, 1H), 4.18-4.05 (m, 1H), 3.87-3.48 (m, 9H), 3.19- 3.05 (m, 1H), 2.99- 2.85 (m, 1H), 2.40-2.30 (m, 1H), 2.28-2.19 (m, 1H), 2.15- 1.52 (m, 17H), 1.47- 1.35 (m, 2H), 1.18-1.08 (m, 3H), 0.82-0.70 (m, 3H), 0.55- 0.38 (m, 2H); 942.6 [M + H]⁺ . |
| | C60 | 1H NMR (400 MHz, DMSO-d6) δ (ppm) 8.80 (d, J = 7.3 Hz, 1H), 8.27 (s, 1H), 8.15-8.00 (m, 2H), 7.60 (d, J = 8.5 Hz, 1H), 7.40- 7.27 (m, 6H), 7.24-7.12 (m, 4H), 6.29-6.09 (m, 2H), 4.80- 4.66 (m, 1H), 4.47 (t, J = 8.4 Hz, 1H), 4.36-4.23 (m, 1H), 4.11 (t, J = 8.4 Hz, 1H), 3.99- 3.88 (m, 2H), 3.82- 3.58 (m, 5H), 3.57-3.49 (m, 2H), 3.20-3.07 (m, 1H), 3.03- 2.88 (m, 1H), 2.40- 2.30 (m, 1H), 2.22 (t, J = 11.4 Hz, 1H), 2.16- 2.05 (m, 5H), 2.03- 1.88 (m, 4H), 1.88-1.48 (m, 10H), 1.14 (d, J = 6.9 Hz, 3H), 0.83 (t, J = 7.4 Hz, 3H), 0.57-0.39 (m, 2H); 942.8 [M + H]⁺ . |
| | C61 | 1H NMR (400 MHz, DMSO-d6) δ (ppm) 10.02-9.32 (m, 1H), 8.99- 8.73 (m, 1H), 8.33- 8.23 (m, 1H), 8.12-8.02 (m, 2H), 7.66-7.55 (m, 1H), 7.41- 7.27 (m, 6H), 7.25- 7.11 (m, 4H), 6.28-6.05 (m, 2H), 4.99-4.71 (m, 3H), 4.61- 4.47 (m, 2H), 4.21- 4.07 (m, 1H), 3.80-3.71 (m, 2H), 3.61-3.49 (m, 2H), 3.44- 2.97 (m, 1H), 2.78- 2.55 (m, 5H), 2.49-1.44 (m, 22H), 1.20-1.03 (m, 3H), 0.63- 0.41 (m, 2H); 957 [M + H]⁺ . |
| | C62 | 1H NMR (400 MHz, DMSO-d6) δ (ppm) 8.87-8.74 (m, 1H), 8.33- 8.23 (m, 1H), 8.09- 8.01 (m, 2H), 7.63-7.55 (m, 1H), 7.38-7.27 (m, 6H), 7.24- 7.10 (m, 4H), 6.31- 6.13 (m, 2H), 4.93-4.66 (m, 3H), 4.52-4.39 (m, 1H), 4.34- 4.22 (m, 1H), 4.18- 4.05 (m, 1H), 3.89-3.68 (m, 2H), 3.62-3.46 (m, 1H), 3.07- 2.92 (m, 1H), 2.66- 2.54 (m, 1H), 2.41-1.49 (m, 27H), 1.20-1.08 (m, 3H), 0.56- 0.38 (m, 2H); 956.6 [M + H]⁺ . |
| | C63 | 1H NMR (400 MHz, DMSO-d6) δ (ppm) 8.81 (d, J = 7.7 Hz, 1H), 8.29- 8.25 (m, 1H), 8.06 (d, J = 8.4 Hz, 1H), 7.99 (s, 1H), 7.51 (d, J = 8.5 Hz, 1H), 7.37-7.28 (m, 4H), 7.24-7.18 (m, 1H), 6.26- 6.01 (m, 2H), 4.91- 4.56 (m, 4H), 4.51-4.43 (m, 1H), 4.34-4.24 (m, 1H), 4.16- 4.08 (m, 1H), 4.05- 3.92 (m, 2H), 3.78-3.61 (m, 2H), 3.59-3.48 (m, 1H), 3.06- 2.95 (m, 1H), 2.64- 2.55 (m, 1H), 2.43-2.30 (m, 2H), 2.27-2.19 (m, 1H), 2.17- 1.45 (m, 20H), 1.27- 1.18 (m, 3H), 0.90-0.81 (m, 3H), 0.55-0.41 (m, 2H); 950.6 [M + H]⁺ . |
| | C64 | 1H NMR (400 MHz, DMSO-d6) δ (ppm) 8.86-8.75 (m, 1H), 8.30- 8.22 (m, 1H), 8.10- 8.02 (m, 1H), 8.01-7.95 (m, 1H), 7.56-7.47 (m, 1H), 7.37- 7.27 (m, 4H), 7.24- 7.18 (m, 1H), 6.27-5.91 (m, 2H), 4.92-4.68 (m, 2H), 4.66- 4.42 (m, 3H), 4.36- 4.24 (m, 1H), 4.16-4.08 (m, 1H), 4.05-3.93 (m, 2H), 3.84- 3.50 (m, 3H), 3.08- 2.93 (m, 1H), 2.64-2.56 (m, 1H), 2.44-2.31 (m, 2H), 2.30- 1.45 (m, 21H), 1.31- 1.01 (m, 3H), 0.89-0.82 (m, 3H), 0.58-0.39 (m, 2H); 950.7 [M + H]⁺ . |
| | C65 | 1H NMR (400 MHz, DMSO-d6) δ (ppm) 8.86-8.75 (m, 1H), 8.27 (s, 1H), 8.10-8.02 (m, 2H), 7.60 (d, J = 8.0 Hz, 1H), 7.39- 7.27 (m, 6H), 7.24-7.13 (m, 4H), 6.28-6.18 (m, 1H), 6.15- 6.05 (m, 1H), 4.71 (m, 1H), 4.48 (m, 1H), 4.34-4.23 (m, 1H), 4.12 (m = 8.6 Hz, 1H), 3.98- 3.89 (m, 1H), 3.87- 3.70 (m, 3H), 3.60-3.49 (m, 1H), 3.07-2.90 (m, 3H), 2.40- 2.28 (m, 2H), 2.26- 2.10 (m, 3H), 2.05-1.91 (m, 7H), 1.90-1.34 (m, 11H), 1.13 (d, J = 7.1 Hz, 3H), 0.86- 0.75 (m, 3H), 0.55-0.42 (m, 2H); 951.0 [M+ H]+ |
| | C66 | 1H NMR (400 MHz, DMSO-d6) δ (ppm) 8.89-8.71 (m, 1H), 8.27 (s, 1H), 8.12-7.99 (m, 2H), 7.65- 7.56 (m, 1H), 7.39- 7.27 (m, 6H), 7.24-7.13 (m, 4H), 6.31-6.21 (m, 1H), 6.18- 6.10 (m, 1H), 4.90- 4.66 (m, 2H), 4.52-4.43 (m, 1H), 4.33-4.24 (m, 1H), 4.15- 3.70 (m, 5H), 3.59- 3.48 (m, 1H), 3.06-2.93 (m, 1H), 2.63-2.53 (m, 3H), 2.49-1.45 (m, 24H), 1.15 (d, J = 7.0 Hz, 3H), 0.60- 0.36 (m, 2H); 1006.7 [M + H]⁺ . |
| | C67 | 1H NMR (400 MHz, DMSO-d6) δ (ppm) 8.85-8.74 (m, 1H), 8.30- 8.23 (m, 1H), 8.08- 8.00 (m, 2H), 7.63-7.54 (m, 1H), 7.40-7.26 (m, 6H), 7.24- 7.10 (m, 4H), 6.30- 6.14 (m, 2H), 4.90-4.67 (m, 2H), 4.52-4.42 (m, 1H), 4.34- 4.21 (m, 1H), 4.15- 3.84 (m, 4H), 3.78-3.67 (m, 1H), 3.58-3.45 (m, 1H), 3.09- 2.94 (m, 1H), 2.66- 2.50 (m, 3H), 2.43-1.48 (m, 24H), 1.22-1.13 (m, 3H), 0.56- 0.38 (m, 2H); 1007.7 [M + H]⁺ . |
| | C68 | 1H NMR (400 MHz, DMSO-d6) δ (ppm) 8.87-8.74 (m, 1H), 8.27 (s, 1H), 8.11-8.03 (m, 2H), 7.60 (d, J = 8.5 Hz, 1H), 7.39 7.27 (m, 6H), 7.24- 7.12 (m, 4H), 6.32- 5.99 (m, 2H), 4.92- 4.63 (m, 3H), 4.53- 4.41 (m, 1H), 4.35- 4.24 (m, 1H), 4.17- 4.07 (m, 1H), 3.85- 3.67 (m, 2H), 3.60- 3.47 (m, 1H), 3.06- 2.95 (m, 1H), 2.64- 2.55 (m, 1H), 2.46- 1.41 (m, 21H), 1.16- 0.98 (m, 9H), 0.57- 0.40 (m, 2H); 944.7 [M + H]⁺ . |
| | C69 | 1H NMR (400 MHz, DMSO-d6) δ (ppm) 8.90-8.71 (m, 1H), 8.34-8.20 (m, 1H), 8.10-8.03 (m, 2H), 7.63-7.57 (m, 1H), 7.40-7.27 (m, 6H), 7.23-7.09 (m, 4H), 6.32-6.04 (m, 2H), 4.91-4.67 (m, 3H), 4.47 (t, J = 8.5 Hz, 1H), 4.34- 4.25 (m, 1H), 4.12 (t, J = 8.6 Hz, 1H), 3.88-3.49 (m, 3H), 3.08-2.94 (m, 1H), 2.65-2.57 (m, 1H), 2.45-2.29 (m, 2H), 2.23 (t, J = 11.6 Hz, 1H), 2.17-2.10 (m, 1H), 2.03-1.94 (m, 5H), 1.89-1.46 (m, 9H), 1.20-1.04 (m, 7H), 0.91 (d, J = 7.1 Hz, 2H), 0.56- 0.39 (m, 2H); 944.7 [M + H]⁺ . |
| | C70 | 1H NMR (400 MHz, DMSO-d6) δ (ppm) 8.78 (d, J = 7.7 Hz, 1H), 8.27 (s, 1H), 8.16 (s, 1H), 8.11-7.92 (m, 2H), 7.60 (d, J = 8.5 Hz, 1H), 7.40 7.09 (m, 10H), 6.30- 5.79 (m, 3H), 4.77- 4.65 (m, 1H), 4.53- 4.41 (m, 1H), 4.34- 4.24 (m, 1H), 4.16- 4.02 (m, 1H), 3.89- 3.64 (m, 4H), 3.60- 3.50 (m, 1H), 3.10- 2.94 (m, 2H), 2.38- 2.09 (m, 5H), 2.03- 1.86 (m, 8H), 1.85- 1.51 (m, 9H), 1.47- 1.35 (m, 2H), 1.13 (d, J = 7.1 Hz, 3H), 0.78 (m, 3H), 0.55- 0.34 (m, 2H); 976.4 [M + H]⁺ . |
| | C71 | 1H NMR (400 MHz, DMSO-d6) δ (ppm) 8.81 (d, J = 7.4 Hz, 1H), 8.27 (s, 1H), 8.11-8.04 (m, 2H), 7.60 (d, J = 8.5 Hz, 1H), 7.39- 7.27 (m, 6H), 7.25-7.14 (m, 4H), 6.29-5.82 (m, 3H), 4.81- 4.66 (m, 1H), 4.50 (t, J = 8.5 Hz, 1H), 4.43-4.29 (m, 1H), 4.13 (t, J = 8.6 Hz, 1H), 3.99- 3.88 (m, 2H), 3.83- 3.68 (m, 2H), 3.60-3.46 (m, 1H), 3.41-3.33 (m, 2H), 2.44- 1.44 (m, 24H), 1.13 (d, J = 7.1 Hz, 3H), 0.83 (m, 3H), 0.56- 0.39 (m, 2H); 976.6 [M + H]⁺ . |
| | C72 | 1H NMR (400 MHz, DMSO-d6) δ (ppm) 9.86-9.42 (m, 1H), 8.93-8.75 (m, 1H), 8.31-8.24 (m, 1H), 8.13-8.03 (m, 2H), 7.64-7.57 (m, 1H), 7.43-7.09 (m, 10H), 6.32 6.07 (m, 2H), 5.27- 4.76 (m, 4H), 4.61- 4.48 (m, 2H), 4.21- 4.08 (m, 1H), 3.93- 3.68 (m, 4H), 3.43- 3.07 (m, 1H), 2.83- 2.56 (m, 5H), 2.50- 1.48 (m, 20H), 1.21- 0.90 (m, 3H), 0.64- 0.43 (m, 2H); 974.5 [M + H]⁺ . |
| | C73 | 1H NMR (400 MHz, DMSO-d6) δ (ppm) 9.92-9.51 (m, 1H), 8.97- 8.72 (m, 1H), 8.30- 8.21 (m, 1H), 8.12-7.98 (m, 2H), 7.61 (d, J = 8.3 Hz, 1H), 7.40- 7.27 (m, 6H), 7.24- 7.09 (m, 4H), 6.28-6.09 (m, 2H), 5.01-4.77 (m, 2H), 4.62- 4.47 (m, 2H), 4.21- 4.09 (m, 1H), 3.96-3.89 (m, 1H), 3.87-3.63 (m, 4H), 3.58- 3.53 (m, 1H), 3.46- 3.03 (m, 1H), 2.82-2.71 (m, 1H), 2.67-2.57 (m, 3H), 2.32- 1.71 (m, 12H), 1.70- 1.50 (m, 2H), 1.45-1.20 (m, 5H), 1.17-1.10 (m, 3H), 0.85- 0.73 (m, 6H), 0.62- 0.46 (m, 2H); 974.9 [M + H]⁺ . |
| | C74 | 1H NMR (400 MHz, DMSO-d6) δ (ppm) 9.69 (d, J = 120.2 Hz, 1H), 8.97-8.72 (m, 1H), 8.27 (d, J = 4.2 Hz, 1H), 8.07 (d, J = 3.3 Hz, 2H), 7.61 (d, J = 8.3 Hz, 1H), 7.40-7.27 (m, 6H), 7.24- 7.09 (m, 4H), 6.28- 6.09 (m, 2H), 4.82 (s, 2H), 4.52 (s, 2H), 4.21-4.09 (m, 1H), 3.96- 3.89 (m, 1H), 3.87- 3.63 (m, 4H), 3.54 (d, J = 7.3 Hz, 1H), 3.46-3.03 (m, 1H), 2.75 (s, 1H), 2.67-2.57 (m, 3H), 2.32- 1.71 (m, 12H), 1.70- 1.50 (m, 2H), 1.45-1.20 (m, 5H), 1.14 (d, J = 6.3 Hz, 3H), 0.85- 0.73 (m, 6H), 0.51 (s, 2H); 987.1 [M + H]⁺ . |
| | C75 | 1H NMR (400 MHz, DMSO-d6) δ (ppm) 9.86-9.44 (m, 1H), 8.97- 8.75 (m, 1H), 8.35- 8.23 (m, 1H), 8.07 (d, J = 6.1 Hz, 2H), 7.68-7.55 (m, 1H), 7.43- 7.03 (m, 10H), 6.33- 6.04 (m, 2H), 5.07-4.71 (m, 2H), 4.67-4.46 (m, 2H), 4.29- 4.06 (m, 1H), 3.85- 3.78 (m, 1H), 3.77-3.72 (m, 1H), 3.71-3.64 (m, 1H), 3.62- 3.47 (m, 2H), 3.45- 3.00 (m, 1H), 2.82-2.71 (m, 1H), 2.63-2.54 (m, 3H), 2.47- 1.30 (m, 18H), 1.25- 1.12 (m, 3H), 0.95-0.74 (m, 9H), 0.64-0.43 (m, 2H); 972.5 [M + H]⁺ . |
| | C76 | 1H NMR (400 MHz, DMSO-d6) δ (ppm) 8.85-8.71 (m, 1H), 8.31- 8.23 (m, 1H), 8.14- 8.00 (m, 2H), 7.61 (d, J = 8.4 Hz, 1H), 7.40-7.26 (m, 6H), 7.23- 7.10 (m, 4H), 6.32- 6.05 (m, 2H), 4.91-4.80 (m, 1H), 4.77-4.64 (m, 2H), 4.53- 4.37 (m, 2H), 4.34- 4.23 (m, 1H), 4.17-4.05 (m, 1H), 3.97-3.62 (m, 2H), 3.59- 3.48 (m, 1H), 3.06- 2.96 (m, 1H), 2.89-2.68 (m, 2H), 2.64-2.56 (m, 1H), 2.45- 1.42 (m, 23H), 1.22- 1.15 (m, 1H), 0.99-0.90 (m, 2H), 0.56-0.36 (m, 2H); 974.9 [M + H]⁺ . |
| | C77 | 1H NMR (400 MHz, DMSO-d6) δ (ppm) 8.80 (s, 1H), 8.27 (d, J = 3.6 Hz, 1H), 8.11-8.02 (m, 2H), 7.61 (d, J = 8.9 Hz, 1H), 7.40- 7.26 (m, 6H), 7.24-7.12 (m, 4H), 6.34-6.07 (m, 2H), 4.96- 4.64 (m, 3H), 4.57- 4.23 (m, 3H), 4.19-4.05 (m, 1H), 3.95-3.66 (m, 2H), 3.59- 3.49 (m, 1H), 3.10- 2.52 (m, 5H), 2.42-1.50 (m, 22H), 1.20-1.08 (m, 3H), 0.58- 0.38 (m, 2H); 974.6 [M + H]⁺ . |
| | C78 | 1H NMR (400 MHz, DMSO-d6) δ (ppm) 8.78 (d, J = 7.6 Hz, 1H), 8.26 (s, 1H), 8.12-7.90 (m, 2H), 7.52 (d, J = 8.5 Hz, 1H), 7.37- 7.27 (m, 4H), 7.24-7.17 (m, 1H), 5.87 (dd, J = 44.3, 6.5 Hz, 1H), 5.58-5.38 (m, 1H), 4.89-4.85 (m, 1H), 4.73- 4.69 (m, 1H), 4.52- 4.41 (m, 1H), 4.34-4.23 (m, 1H), 4.15-4.01 (m, 3H), 3.79- 3.67 (m, 1H), 3.59- 3.46 (m, 2H), 3.07-2.94 (m, 1H), 2.63-2.55 (m, 1H), 2.43- 1.48 (m, 22H), 1.25 (t, J = 7.0 Hz, 3H), 1.20-1.11 (m, 6H), 0.97 (d, J = 7.2 Hz, 3H), 0.56- 0.40 (m, 2H); 986.7 [M + H]⁺ . |
| | C79 | 1H NMR (400 MHz, DMSO-d6) δ (ppm) 9.90-9.47 (m, 1H), 8.92- 8.74 (m, 1H), 8.30- 8.23 (m, 1H), 8.11-8.03 (m, 2H), 7.60 (t, J = 8.2 Hz, 1H), 7.40- 7.27 (m, 6H), 7.26- 7.10 (m, 4H), 6.30-6.06 (m, 2H), 5.05-4.76 (m, 2H), 4.62- 4.47 (m, 2H), 4.20- 4.07 (m, 1H), 3.96-3.67 (m, 5H), 3.58-3.53 (m, 1.5H), 3.13 3.00 (m, 0.5H), 2.83-2.55 (m, 5H), 2.49-2.34 (m, 2H), 2.30- 1.48 (m, 14H), 1.45- 1.33 (m, 1H), 1.30-1.20 (m, 4H), 1.19-0.92 (m, 3H), 0.85- 0.72 (m, 6H), 0.62- 0.43 (m, 2H); 986.6 [M + H]⁺ . |
| | C80 | 1H NMR (400 MHz, DMSO-d6) δ (ppm) 9.82-9.43 (m, 1H), 8.95- 8.74 (m, 1H), 8.30- 8.25 (m, 1H), 8.17-8.03 (m, 2H), 7.71-7.56 (m, 1H), 7.39- 7.28 (m, 6H), 7.23- 7.06 (m, 4H), 6.31-6.07 (m, 2H), 5.03-4.73 (m, 2H), 4.65- 4.47 (m, 2H), 4.21- 4.11 (m, 1H), 3.83-3.69 (m, 4H), 3.63-3.52 (m, 2H), 3.43 3.02 (m, 1H), 2.82- 2.62 (m, 2H), 2.61-2.55 (m, 3H), 2.49-1.45 (m, 16H), 1.23- 0.95 (m, 3H), 0.83 (s, 8H), 0.64-0.44 (m, 2H); 973.0 [M + H]⁺ . |
| | C81 | 1H NMR (400 MHz, DMSO-d6) δ (ppm) 8.80 (d, J = 7.5 Hz, 1H), 8.27 (s, 1H), 8.11-7.99 (m, 2H), 7.60 (d, J = 8.7 Hz, 1H), 7.43- 7.26 (m, 6H), 7.23-7.08 (m, 4H), 6.29-6.04 (m, 2H), 4.94- 4.66 (m, 2H), 4.53- 4.41 (m, 1H), 4.29 (s, 1H), 4.19- 4.04 (m, 1H), 3.99- 3.87 (m, 1H), 3.86-3.67 (m, 3H), 3.60-3.46 (m, 1H), 3.00 (s, 1H), 2.69-2.57 (m, 1H), 2.33 (s, 2H), 2.27-1.48 (m, 20H), 1.45- 1.33 (m, 1H), 1.18- 1.07 (m, 3H), 0.88-0.70 (m, 3H), 0.57-0.40 (m, 2H); 944.6 [M + H]⁺ . |
| | C82 | 1H NMR (400 MHz, DMSO-d6) δ (ppm) 8.77 (d, J = 7.6 Hz, 1H), 8.27 (s, 1H), 8.10-8.00 (m, 2H), 7.61 (d, J = 8.5 Hz, 1H), 7.40- 7.26 (m, 6H), 7.24-7.11 (m, 4H), 6.26-6.04 (m, 2H), 4.85- 4.71 (m, 1H), 4.52- 4.42 (m, 1H), 4.35-4.25 (m, 1H), 4.17-4.06 (m, 1H), 3.87- 3.67 (m, 4H), 3.57- 3.48 (m, 1H), 2.99-2.87 (m, 1H), 2.46-2.42 (m, 1H), 2.33- 1.55 (m, 22H), 1.46- 1.36 (m, 2H), 1.13 (d, J = 7.1 Hz, 3H), 0.83-0.70 (m, 3H), 0.53- 0.37 (m, 2H); 982.5 [M + H]⁺ . |
| | C83 | 1H NMR (400 MHz, DMSO-d6) δ (ppm) 8.79 (d, J = 7.7 Hz, 1H), 8.25 (s, 1H), 8.01 (d, J = 8.4 Hz, 1H), 7.96 (s, 1H), 7.51 (d, J = 8.5 Hz, 1H), 7.36- 7.28 (m, 4H), 7.24- 7.18 (m, 1H), 6.01 (dd, J = 44.1, 7.0 Hz, 1H), 5.68- 5.56 (m, 1H), 4.95- 4.65 (m, 3H), 4.54-4.43 (m, 1H), 4.33-4.24 (m, 1H), 4.15- 4.08 (m, 1H), 4.00- 3.89 (m, 2H), 3.77-3.66 (m, 2H), 3.58-3.49 (m, 1H), 3.06- 2.94 (m, 1H), 2.63- 2.55 (m, 1H), 2.44-2.29 (m, 2H), 2.26-2.19 (m, 1H), 2.17- 1.89 (m, 9H), 1.88- 1.76 (m, 3H), 1.74-1.49 (m, 6H), 1.25 (d, J = 7.1 Hz, 3H), 1.19- 1.10 (m, 9H), 0.54- 0.42 (m, 2H); 896.6 [M + H]⁺ . |
| | C84 | 1H NMR (400 MHz, DMSO-d6) δ (ppm) 8.85-8.73 (m, 1H), 8.27 (d, J = 4.2 Hz, 1H), 8.11- 8.01 (m, 2H), 7.64-7.54 (m, 1H), 7.40-7.27 (m, 6H), 7.24- 7.11 (m, 4H), 6.30- 6.08 (m, 2H), 4.91-4.65 (m, 2H), 4.52-4.41 (m, 1H), 4.29 (s, 1H), 4.17-4.07 (m, 1H), 3.96- 3.67 (m, 4H), 3.59- 3.46 (m, 1H), 3.05-2.93 (m, 1H), 2.64-2.54 (m, 1H), 2.44- 2.30 (m, 2H), 2.27- 2.03 (m, 3H), 2.02-1.92 (m, 6H), 1.89-1.75 (m, 3H), 1.74- 1.43 (m, 8H), 1.19- 0.94 (m, 3H), 0.84-0.74 (m, 3H), 0.55-0.40 (m, 2H); 944.6 [M + H]⁺ . |
| | C85 | 1H NMR (400 MHz, DMSO-d6) δ (ppm) 8.84-8.71 (m, 1H), 8.27 (s, 1H), 8.10-8.03 (m, 2H), 7.61 (d, J = 8.1 Hz, 1H), 7.36 (t, J = 7.8 Hz, 2H), 7.21-7.11 (m, 3H), 6.27-6.06 (m, 2H), 4.90- 4.65 (m, 2H), 4.45 (t, J = 8.5 Hz, 1H), 4.33-4.23 (m, 1H), 3.97-3.73 (m, 4H), 3.64- 3.54 (m, 1H), 2.97 (s, 1H), 2.65-2.55 (m, 1H), 2.41- 2.24 (m, 2H), 2.13- 1.92 (m, 6H), 1.91-1.37 (m, 17H), 1.17-1.09 (m, 3H), 0.86- 0.75 (m, 3H), 0.46- 0.35 (m, 2H); 868.5 [M + H]⁺ . |
| | C86 | 1H NMR (400 MHz, DMSO-d6) δ (ppm) 8.89-8.66 (m, 1H), 8.25 (s, 1H), 8.09-7.89 (m, 2H), 7.53 (d, J = 8.5 Hz, 1H), 7.38- 7.27 (m, 4H), 7.25-7.18 (m, 1H), 6.06-5.82 (m, 1H), 5.64- 5.47 (m, 1H), 4.91- 4.63 (m, 3H), 4.55-4.41 (m, 1H), 4.37-4.22 (m, 1H), 4.17- 4.06 (m, 1H), 4.03- 3.90 (m, 2H), 3.80-3.65 (m, 2H), 3.59-3.48 (m, 1H), 3.06- 2.92 (m, 1H), 2.63- 2.55 (m, 1H), 2.49-1.46 (m, 21H), 1.26-1.06 (m, 12H), 0.59- 0.38 (m, 2H); 896.7 [M + H]⁺ . |
| | C87 | 1H NMR (400 MHz, DMSO-d6) δ (ppm) 8.84-8.72 (m, 1H), 8.26 (s, 1H), 8.11-8.02 (m, 1H), 7.98 (d, J = 9.6 Hz, 1H), 7.58- 7.46 (m, 1H), 7.39-7.26 (m, 4H), 7.24-7.19 (m, 1H), 6.27- 5.92 (m, 2H), 4.84- 4.73 (m, 1H), 4.65-4.42 (m, 3H), 4.36-4.26 (m, 1H), 4.16- 4.06 (m, 1H), 4.05- 3.93 (m, 2H), 3.84-3.63 (m, 2H), 3.58-3.49 (m, 1H), 3.01- 2.89 (m, 1H), 2.47- 2.43 (m, 1H), 2.35-2.20 (m, 4H), 2.16-2.08 (m, 4H), 2.04- 1.94 (m, 3H), 1.90- 1.48 (m, 13H), 1.31-1.02 (m, 3H), 0.89-0.80 (m, 3H), 0.54 0.39 (m, 2H); 988.5 [M + H]⁺ . |
| | C88 | 1H NMR (400 MHz, DMSO-d6) δ (ppm) 8.79 (d, J = 7.7 Hz, 1H), 8.25 (d, J = 3.0 Hz, 1H), 8.08-7.95 (m, 2H), 7.56-7.48 (m, 1H), 7.38- 7.17 (m, 5H), 6.07- 5.88 (m, 1H), 5.69-5.53 (m, 1H), 4.90-4.67 (m, 2H), 4.53- 4.41 (m, 1H), 4.34- 4.23 (m, 1H), 4.16-4.08 (m, 1H), 4.05-3.81 (m, 4H), 3.80- 3.47 (m, 3H), 3.05- 2.93 (m, 1H), 2.65-2.55 (m, 1H), 2.46-1.46 (m, 25H), 1.29- 1.17 (m, 3H), 0.93- 0.73 (m, 6H), 0.57-0.36 (m, 2H); 910.5 [M + H]⁺ . |
| | C89 | 1H NMR (400 MHz, DMSO-d6) δ (ppm) 8.81 (m 7.5 Hz, 1H), 8.27 (s, 1H), 8.10-7.93 (m, 2H), 7.51 (m, 1H), 7.39-7.26 (m, 4H), 7.25- 7.16 (m, 1H), 6.28- 5.89 (m, 2H), 4.96-4.39 (m, 6H), 4.35-4.22 (m, 1H), 4.17- 4.06 (m, 1H), 3.85- 3.48 (m, 3H), 3.00 (s, 1H), 2.67- 2.56 (m, 1H), 2.45- 1.39 (m, 27H), 1.30-0.98 (m, 3H), 0.55-0.40 (m, 2H); 962.5 [M + H]⁺ . |
| | C90 | 1H NMR (400 MHz, DMSO-d6) δ (ppm) 8.82-8.74 (m, 1H), 8.30- 8.23 (m, 1H), 8.09- 7.97 (m, 2H), 7.56-7.47 (m, 1H), 7.39-7.27 (m, 4H), 7.24- 7.18 (m, 1H), 6.24- 5.88 (m, 2H), 4.78-4.69 (m, 1H), 4.64-4.41 (m, 3H), 4.37- 4.24 (m, 1H), 4.15- 3.50 (m, 6H), 3.12-2.95 (m, 2H), 2.72-2.57 (m, 1H), 2.42- 2.28 (m, 1H), 2.25- 2.05 (m, 6H), 2.02-1.67 (m, 12H), 1.64-1.42 (m, 5H), 1.28- 1.17 (m, 3H), 0.90- 0.76 (m, 3H), 0.52-0.38 (m, 2H); 982.5 [M + H]⁺ . |
| | C91 | 1H NMR (400 MHz, DMSO-d6) δ (ppm) 8.88-8.68 (m, 1H), 8.34- 8.19 (m, 1H), 8.11- 7.95 (m, 2H), 7.58-7.46 (m, 1H), 7.38-7.17 (m, 5H), 6.24- 5.93 (m, 2H), 4.86- 4.70 (m, 1H), 4.66-4.43 (m, 3H), 4.35-4.25 (m, 1H), 4.15- 3.50 (m, 6H), 3.01- 2.87 (m, 1H), 2.44-2.20 (m, 6H), 2.14-2.10 (m, 3H), 2.05- 1.92 (m, 3H), 1.88- 1.44 (m, 13H), 1.28-1.20 (m, 3H), 0.89-0.77 (m, 3H), 0.54- 0.37 (m, 2H); 988.7 [M + H]⁺ . |
| | C92 | 1H NMR (400 MHz, DMSO-d6) δ (ppm) 9.04-8.72 (m, 1H), 8.25 (s, 1H), 8.07-7.95 (m, 2H), 7.51 (d, J = 8.2 Hz, 1H), 7.38- 7.28 (m, 4H), 7.25-7.19 (m, 1H), 6.06-5.55 (m, 2H), 5.05- 4.71 (m, 2H), 4.64- 4.23 (m, 2H), 4.20-4.10 (m, 1H), 4.01-3.52 (m, 7H), 3.15- 2.90 (m, 1H), 2.84- 2.55 (m, 4H), 2.39-1.40 (m, 19H), 1.31-1.19 (m, 3H), 0.88- 0.78 (m, 3H), 0.78- 0.63 (m, 2H), 0.62-0.44 (m, 4H); 908.7 [M + H]⁺ . |
| | C93 | 1H NMR (400 MHz, DMSO-d6) δ (ppm) 8.89-8.70 (m, 1H), 8.26 (s, 1H), 8.10-7.90 (m, 2H), 7.59- 7.45 (m, 1H), 7.40- 7.17 (m, 5H), 6.07-5.84 (m, 1H), 5.71-5.54 (m, 1H), 4.93- 4.66 (m, 3H), 4.56- 4.42 (m, 1H), 4.34-4.23 (m, 1H), 4.17-4.00 (m, 3H), 3.78- 3.69 (m, 1H), 3.58- 3.50 (m, 2H), 3.05-2.97 (m, 1H), 2.63-2.56 (m, 1H), 2.43- 2.31 (m, 2H), 2.27- 2.19 (m, 1H), 2.17-1.46 (m, 18H), 1.28-1.11 (m, 12H), 0.59- 0.38 (m, 2H); 896.7 [M + H]⁺ . |
| | C94 | 1H NMR (400 MHz, DMSO-d6) δ (ppm) 9.65-9.40 (m, 1H), 8.85 (m, 1H), 8.31-8.22 (m, 1H), 8.12- 7.94 (m, 2H), 7.53 (d, J = 8.6 Hz, 1H), 7.39-7.28 (m, 4H), 7.25-7.19 (m, 1H), 6.27- 5.91 (m, 2H), 5.08- 4.77 (m, 3H), 4.71-4.40 (m, 4H), 4.23-4.04 (m, 1H), 3.95- 3.68 (m, 2H), 3.66- 3.48 (m, 2H), 3.46-2.99 (m, 1H), 2.83-2.65 (m, 2H), 2.64- 2.54 (m, 3H), 2.47- 2.38 (m, 2H), 2.34-1.48 (m, 14H), 1.29-1.06 (m, 9H), 0.66- 0.43 (m, 2H); 950.1 [M + H]⁺ . |
| | C95 | 1H NMR (400 MHz, DMSO-d6) δ (ppm) 8.85-8.71 (m, 1H), 8.31- 8.22 (m, 1H), 8.11- 7.94 (m, 2H), 7.56-7.46 (m, 1H), 7.37-7.28 (m, 4H), 7.24- 7.15 (m, 1H), 6.28- 5.90 (m, 2H), 4.79-4.68 (m, 1H), 4.66-4.41 (m, 3H), 4.35- 4.25 (m, 1H), 4.14- 3.62 (m, 5H), 3.58-3.47 (m, 1H), 3.11-2.93 (m, 2H), 2.72- 2.58 (m, 1H), 2.40- 2.32 (m, 1H), 2.25-2.06 (m, 6H), 2.02-1.68 (m, 12H), 1.64- 1.48 (m, 5H), 1.30- 1.00 (m, 3H), 0.89-0.81 (m, 3H), 0.52-0.38 (m, 2H); 982.5 [M + H]⁺ . |
| | C96 | 1H NMR (400 MHz, DMSO-d6) δ (ppm) 8.98-8.58 (m, 1H), 8.38- 8.14 (m, 1H), 8.13- 7.83 (m, 2H), 7.60-7.47 (m, 1H), 7.41-7.07 (m, 5H), 6.27- 5.89 (m, 2H), 4.87- 4.40 (m, 5H), 4.36-4.24 (m, 1H), 4.20-3.46 (m, 6H), 3.06- 2.91 (m, 1H), 2.75- 2.57 (m, 2H), 2.48-1.42 (m, 22H), 1.34-1.13 (m, 3H), 0.95- 0.70 (m, 3H), 0.59- 0.29 (m, 2H); 982.5 [M + H]⁺ . |
| | C97 | 1H NMR (400 MHz, DMSO-d6) δ (ppm) 8.88-8.62 (m, 1H), 8.37- 8.12 (m, 1H), 8.11- 7.82 (m, 2H), 7.59-7.43 (m, 1H), 7.40-7.08 (m, 5H), 6.33- 5.82 (m, 2H), 4.81- 4.34 (m, 5H), 4.34-4.20 (m, 1H), 4.17-4.07 (m, 1H), 4.05- 3.88 (m, 2H), 3.86- 3.47 (m, 3H), 3.07-2.89 (m, 1H), 2.75-2.57 (m, 2H), 2.46- 2.08 (m, 5H), 2.05- 1.39 (m, 17H), 1.32-0.98 (m, 3H), 0.93-0.72 (m, 3H), 0.60- 0.29 (m, 2H); 1016.9 [M + H]⁺ . |
| | C98 | 1H NMR (400 MHz, DMSO-d6) δ (ppm) 8.85-8.74 (m, 1H), 8.27 (s, 1H), 8.11-8.01 (m, 2H), 7.65- 7.57 (m, 1H), 7.42- 7.26 (m, 6H), 7.24-7.11 (m, 4H), 6.30-6.04 (m, 2H), 4.78- 4.65 (m, 1H), 4.50- 4.38 (m, 3H), 4.34-4.25 (m, 1H), 4.21-4.16 (m, 1H), 4.15- 4.06 (m, 2H), 3.99- 3.89 (m, 1H), 3.87-3.67 (m, 3H), 3.59-3.48 (m, 1H), 2.93- 2.81 (m, 1H), 2.48- 2.43 (m, 1H), 2.41-2.31 (m, 2H), 2.29-2.16 (m, 2H), 2.14- 1.36 (m, 15H), 1.29 (s, 3H), 1.18-1.09 (m, 3H), 0.94 (t, J = 6.9 Hz, 3H), 0.85- 0.75 (m, 3H), 0.53-0.42 (m, 2H); 970.4 [M + H]⁺ . |
| | C99 | 1H NMR (400 MHz, DMSO-d6) δ (ppm) 8.85-8.76 (m, 1H), 8.27 (s, 1H), 8.06 (d, J = 4.8 Hz, 2H), 7.60 (d, J = 7.2 Hz, 1H), 7.39- 7.27 (m, 6H), 7.23- 7.13 (m, 4H), 6.28- 6.06 (m, 2H), 4.76- 4.65 (m, 1H), 4.54- 4.43 (m, 2H), 4.34- 4.23 (m, 1H), 4.13 (t, J = 8.7 Hz, 1H), 3.97-3.89 (m, 1H), 3.85-3.70 (m, 3H), 3.59-3.49 (m, 1H), 3.08-2.99 (m, 1H), 2.89-2.79 (m, 1H), 2.63-2.53 (m, 1H), 2.37-1.90 (m, 10H), 1.88-1.38 (m, 11H), 1.13 (d, J = 7.0 Hz, 3H), 0.97 (t, J = 7.1 Hz, 3H), 0.85-0.74 (m, 3H), 0.52-0.39 (m, 2H); 1024.7 [M + H]⁺ . |
| | C100 | 1H NMR (400 MHz, DMSO-d6) δ (ppm) 8.82-8.75 (m, 1H), 8.27 (s, 1H), 8.09-8.03 (m, 2H), 7.63-7.57 (m, 1H), 7.38-7.27 (m, 6H), 7.24-7.14 (m, 4H), 6.30-6.04 (m, 2H), 4.78-4.67 (m, 1H), 4.50-4.42 (m, 1H), 4.33-4.25 (m, 1H), 4.16-4.06 (m, 1H), 3.95-3.67 (m, 4H), 3.58-3.48 (m, 1H), 3.07-2.97 (m, 1H), 2.48-2.09 (m, 9H), 2.04-1.92 (m, 3H), 1.84-1.39 (m, 12H), 1.16-1.08 (m, 3H), 0.96 (t, J = 7.0 Hz, 3H), 0.86- 0.74 (m, 3H), 0.53- 0.38 (m, 2H); 996.6 [M + H]⁺ . |
| | C101 | 1H NMR (400 MHz, DMSO-d6) δ (ppm) 8.83-8.74 (m, 1H), 8.29- 8.24 (m, 1H), 8.11- 8.01 (m, 2H), 7.65-7.55 (m, 1H), 7.39-7.27 (m, 6H), 7.24- 7.11 (m, 4H), 6.31- 6.08 (m, 2H), 4.78-4.67 (m, 1H), 4.51-4.43 (m, 1H), 4.35- 4.25 (m, 1H), 4.16- 4.06 (m, 1H), 3.95-3.65 (m, 4H), 3.58-3.48 (m, 1H), 3.07- 2.96 (m, 1H), 2.48- 2.16 (m, 8H), 2.15-2.08 (m, 1H), 2.04-1.92 (m, 3H), 1.85- 1.43 (m, 12H), 1.20- 0.92 (m, 6H), 0.84-0.77 (m, 3H), 0.53-0.38 (m, 2H); 996.6 [M + H]⁺ . |
| | C102 | 1H NMR (400 MHz, DMSO-d6) δ (ppm) 8.85-8.76 (m, 1H), 8.29- 8.22 (m, 1H), 8.08- 8.03 (m, 1H), 8.00-7.98 (m, 1H), 7.55-7.49 (m, 1H), 7.37- 7.27 (m, 4H), 7.24- 7.18 (m, 1H), 6.23-5.93 (m, 2H), 4.94-4.44 (m, 6H), 4.33- 4.25 (m, 1H), 4.12 (t, J = 8.8 Hz, 1H), 3.83-3.47 (m, 3H), 3.05-2.90 (m, 1H), 2.65- 2.55 (m, 1H), 2.41- 2.30 (m, 2H), 2.28-2.10 (m, 4H), 2.05-1.47 (m, 21H), 1.26- 1.17 (m, 3H), 0.55- 0.40 (m, 2H); 962.5 [M + H]⁺ . |
| | C103 | 1H NMR (400 MHz, DMSO-d6) δ (ppm) 9.06-8.65 (m, 1H), 8.26 (s, 1H), 8.12-7.96 (m, 2H), 7.56- 7.49 (m, 1H), 7.39- 7.27 (m, 4H), 7.25-7.19 (m, 1H), 6.29-5.89 (m, 2H), 5.02- 4.71 (m, 3H), 4.68- 4.34 (m, 4H), 4.21-4.07 (m, 1H), 3.93-3.75 (m, 1H), 3.74- 3.48 (m, 3H), 2.84- 2.54 (m, 5H), 2.33-1.51 (m, 17H), 1.31-1.24 (m, 1H), 1.20- 1.10 (m, 6H), 1.09- 0.96 (m, 2H), 0.64-0.42 (m, 2H); 950.5 [M+ H]+ |
| | C104 | 1H NMR (400 MHz, DMSO-d6) δ (ppm) 9.88-9.41 (m, 1H), 8.95- 8.74 (m, 1H), 8.27 (d, J = 5.1 Hz, 1H), 8.13-7.97 (m, 2H), 7.61 (d, J = 8.3 Hz, 1H), 7.41- 7.26 (m, 6H), 7.25- 7.13 (m, 4H), 6.29-6.08 (m, 2H), 5.04-4.73 (m, 2H), 4.64- 4.41 (m, 2H), 4.23- 4.03 (m, 1H), 3.90-3.64 (m, 4H), 3.62-3.02 (m, 3H), 2.81- 2.54 (m, 5H), 2.49- 1.46 (m, 17H), 1.21-1.04 (m, 3H), 0.90-0.70 (m, 6H), 0.63- 0.40 (m, 2H); 958.7 [M + H]⁺ . |
| | C105 | 1H NMR (400 MHz, DMSO-d6) δ (ppm) 9.86-9.40 (m, 1H), 8.96- 8.70 (m, 1H), 8.31- 8.20 (m, 1H), 8.14-7.97 (m, 2H), 7.68-7.50 (m, 1H), 7.44- 7.06 (m, 10H), 6.30- 6.08 (m, 2H), 5.00-4.78 (m, 2H), 4.62-4.47 (m, 2H), 4.21- 4.07 (m, 1H), 3.80- 3.67 (m, 4H), 3.67-2.93 (m, 3H), 2.81-2.54 (m, 5H), 2.49- 1.50 (m, 17H), 1.23- 0.90 (m, 3H), 0.89-0.72 (m, 6H), 0.64-0.41 (m, 2H); 958.6 [M + H]⁺ . |
| | C106 | 1H NMR (400 MHz, DMSO-d6) δ (ppm) 10.00-9.43 (m, 1H), 8.95- 8.76 (m, 1H), 8.31- 8.22 (m, 1H), 8.08-7.94 (m, 2H), 7.56-7.48 (m, 1H), 7.39- 7.28 (m, 4H), 7.27- 7.18 (m, 1H), 6.09-5.60 (m, 2H), 5.05-4.74 (m, 4H), 4.66- 4.49 (m, 2H), 4.22- 3.92 (m, 4H), 3.85-3.73 (m, 1H), 3.61-3.54 (m, 1H), 3.44- 2.98 (m, 1H), 2.84- 2.63 (m, 2H), 2.64-2.55 (m, 3H), 2.47-2.39 (m, 1H), 2.34- 1.51 (m, 21H), 1.29- 1.17 (m, 6H), 0.64-0.44 (m, 2H); 908.5 [M + H]⁺ . |
| | C107 | 1H NMR (400 MHz, DMSO-d6) δ (ppm) 10.13-9.39 (m, 1H), 8.95- 8.72 (m, 1H), 8.31- 8.22 (m, 1H), 8.12-7.93 (m, 2H), 7.52 (t, J = 7.9 Hz, 1H), 7.40- 7.30 (m, 4H), 7.27- 7.18 (m, 1H), 6.11-5.48 (m, 2H), 5.02-4.77 (m, 3H), 4.62- 4.48 (m, 2H), 4.22- 4.05 (m,2H), 4.00- 3.90 (m, 1H), 3.85-3.74 (m, 2H), 3.65-3.53 (m, 2H), 3.45- 2.97 (m, 1H), 2.80- 2.60 (m, 2H), 2.60-2.53 (m, 3H), 2.49-1.46 (m, 22H), 1.31- 0.93 (m, 6H), 0.68- 0.41 (m, 1H); 908.6 [M + H]⁺ . |
| | C108 | 1H NMR (400 MHz, DMSO-d6) δ (ppm) 9.73-9.14 (m, 1H), 8.92- 8.77 (m, 1H), 8.27 (s, 1H), 8.13-8.02 (m, 2H), 7.62 (d, J = 8.3 Hz, 1H), 7.39- 7.09 (m, 10H), 6.29-6.07 (m, 2H), 4.83-4.76 (m, 1H), 4.59- 4.50 (m, 2H), 4.20- 4.10 (m, 1H), 3.98-3.91 (m, 1H), 3.84-3.75 (m, 2H), 3.60- 3.47 (m, 1H), 3.22- 2.88 (m, 4H), 2.76-2.53 (m, 2H), 2.47-1.02 (m, 26H), 0.86- 0.72 (m, 3H), 0.61- 0.38 (m, 2H); 1009.4 [M + H]⁺ . |
| | C109 | 1H NMR (400 MHz, DMSO-d6) δ (ppm) 8.93-8.79 (m, 1H), 8.33- 8.20 (m, 1H), 8.13- 8.01 (m, 2H), 7.67-7.56 (m, 1H), 7.39-7.09 (m, 10H), 6.30- 6.08 (m, 2H), 4.86- 4.73 (m, 1H), 4.61-4.47 (m, 2H), 4.20-4.07 (m, 1H), 4.01- 3.80 (m, 4H), 3.79- 3.65 (m, 2H), 3.63-3.45 (m, 1H), 3.21-3.10 (m, 1H), 3.08- 2.84 (m, 2H), 2.77- 2.54 (m, 2H), 2.46-0.92 (m, 24H), 0.85-0.76 (m, 3H), 0.62- 0.36 (m, 2H); 1008.5 [M + H]⁺ . |
| | C110 | 1H NMR (400 MHz, DMSO-d6) δ (ppm) 9.94-9.42 (m, 1H), 8.99- 8.71 (m, 1H), 8.33- 8.16 (m, 1H), 8.14-7.93 (m, 2H), 7.67-7.47 (m, 1H), 7.42- 7.27 (m, 4H), 7.26- 7.14 (m, 1H), 6.38-5.86 (m, 2H), 4.86-4.75 (m, 1H), 4.66- 4.47 (m, 4H), 4.20- 4.09 (m, 2H), 4.04-3.89 (m, 4H), 3.87-3.77 (m, 2H), 3.75- 3.62 (m, 2H), 3.59- 3.51 (m, 1H), 3.27-3.13 (m, 1H), 2.74-2.62 (m, 2H), 2.61- 2.53 (m, 4H), 2.49- 2.42 (m, 1H), 2.29-2.19 (m, 1H), 2.15-1.97 (m, 5H), 1.90- 1.74 (m, 4H), 1.60- 1.45 (m, 3H), 1.32-1.16 (m, 3H), 0.91-0.77 (m, 3H), 0.66- 0.38 (m, 2H); 957.5 [M + H]⁺ . |
| | C111 | 1H NMR (400 MHz, DMSO-d6) δ (ppm) 8.88-8.72 (m, 1H), 8.34- 8.23 (m, 1H), 8.12- 7.99 (m, 2H), 7.67-7.56 (m, 1H), 7.41-7.26 (m, 6H), 7.25- 7.07 (m, 4H), 6.31- 6.03 (m, 2H), 4.93-4.65 (m, 2H), 4.56-4.40 (m, 1H), 4.36- 4.21 (m, 1H), 4.19- 4.07 (m, 1H), 3.88-3.65 (m, 2H), 3.61-3.46 (m, 1H), 3.10- 2.87 (m, 3H), 2.41- 1.39 (m, 27H), 1.21-1.03 (m, 3H), 0.62-0.37 (m, 2H); 964.4 [M + H]⁺ . |
| | C112 | 1H NMR (400 MHz, DMSO-d6) δ (ppm) 8.80 (t, J = 7.1 Hz, 1H), 8.30- 8.24 (m, 1H), 8.10- 8.03 (m, 2H), 7.63-7.57 (m, 1H), 7.40-7.27 (m, 6H), 7.24- 7.10 (m, 4H), 6.34- 6.00 (m, 2H), 4.87-4.65 (m, 2H), 4.47 (t, J = 8.4 Hz, 1H), 4.33- 4.25 (m, 1H), 4.12 (t, J = 8.5 Hz, 1H), 3.88-3.48 (m, 3H), 3.07-2.89 (m, 3H), 2.38- 2.29 (m, 2H), 2.26- 2.11 (m, 5H), 2.05-1.47 (m, 20H), 1.20-0.87 (m, 3H), 0.55- 0.42 (m, 2H); 963.6 [M + H]⁺ . |
| | C113 | 1H NMR (400 MHz, DMSO-d6) δ (ppm) 8.85-8.74 (m, 1H), 8.27 (s, 1H), 8.09-8.02 (m, 2H), 7.63- 7.56 (m, 1H), 7.39- 7.27 (m, 6H), 7.24-7.12 (m, 4H), 6.28-6.05 (m, 2H), 4.88- 4.67 (m, 2H), 4.51- 4.44 (m, 1H), 4.34-4.25 (m, 1H), 4.16-4.07 (m, 1H), 3.86- 3.68 (m, 2H), 3.59- 3.48 (m, 1H), 3.06-2.94 (m, 2H), 2.84-2.70 (m, 1H), 2.32- 1.49 (m, 27H), 1.18- 1.08 (m, 3H), 0.52-0.38 (m, 2H); 1006.7 [M + H]⁺ . |
| | C114 | 1H NMR (400 MHz, DMSO-d6) δ (ppm) 8.80 (d, J = 7.7 Hz, 1H), 8.27 (d, J = 3.9 Hz, 1H), 8.09-8.03 (m, 1H), 8.00 (s, 1H), 7.58-7.47 (m, 1H), 7.38-7.26 (m, 4H), 7.24- 7.18 (m, 1H), 6.24- 5.92 (m, 2H), 4.77-4.42 (m, 4H), 4.35-4.24 (m, 1H), 4.18- 4.06 (m, 1H), 4.04- 3.62 (m, 4H), 3.59-3.49 (m, 1H), 3.08-2.89 (m, 2H), 2.84- 2.70 (m, 1H), 2.39- 2.18 (m, 3H), 2.16-2.04 (m, 3H), 2.03-1.89 (m, 6H), 1.87- 1.44 (m, 11H), 1.29- 1.19 (m, 3H), 0.90-0.76 (m, 3H), 0.54-0.33 (m, 2H); 1000.6 [M + H]⁺ . |
| | C115 | 1H NMR (400 MHz, DMSO-d6) δ (ppm) 8.96-8.68 (m, 1H), 8.28 (s, 1H), 8.15-8.03 (m, 2H), 7.62 (d, J = 8.4 Hz, 1H), 7.42- 7.27 (m, 6H), 7.24-7.15 (m, 4H), 6.28-6.04 (m, 2H), 4.88- 4.74 (m, 1H), 4.61- 4.43 (m, 2H), 4.18-4.07 (m, 1H), 3.97-3.85 (m, 2H), 3.76- 3.66 (m, 2H), 3.59- 3.48 (m, 1H), 3.22-2.93 (m, 2H), 2.80-2.55 (m, 3H), 2.47- 2.42 (m, 1H), 2.41- 2.29 (m, 2H), 2.26-2.18 (m, 1H), 2.15-1.68 (m, 14H), 1.59- 1.52 (m, 1H), 1.52- 1.44 (m, 2H), 0.94 (d, J = 7.0 Hz, 3H), 0.81 (t, J = 7.4 Hz, 3H), 0.58- 0.42 (m, 2H); 982.5 [M + H]⁺ . |
| | C116 | 1H NMR (400 MHz, DMSO-d6) δ (ppm) 8.85-8.71 (m, 1H), 8.29- 8.18 (m, 1H), 8.07- 7.91 (m, 2H), 7.50 (d, J = 8.6 Hz, 1H), 7.41-7.27 (m, 4H), 7.25- 7.18 (m, 1H), 6.10- 5.61 (m, 2H), 4.79-4.63 (m, 1H), 4.58-4.42 (m, 2H), 4.36- 4.24 (m, 1H), 4.12 (t, J = 8.5 Hz, 1H), 4.05-3.94 (m, 1H), 3.92-3.85 (m, 1H), 3.84- 3.70 (m, 2H), 3.61- 3.48 (m, 1H), 3.05-2.94 (m, 1H), 2.73-2.58 (m, 2H), 2.46- 2.29 (m, 2H), 2.27- 2.18 (m, 1H), 2.17-2.09 (m, 2H), 2.03-1.40 (m, 18H), 1.29- 1.16 (m, 3H), 0.89- 0.55 (m, 7H), 0.53-0.39 (m, 2H); 975.3 [M + H]⁺ . |
| | C117 | 1H NMR (400 MHz, DMSO-d6) δ (ppm) 8.82-8.73 (m, 1H), 8.28- 8.21 (m, 1H), 8.06- 7.92 (m, 2H), 7.50 (d, J = 8.4 Hz, 1H), 7.39-7.26 (m, 4H), 7.25- 7.18 (m, 1H), 6.08- 5.61 (m, 2H), 4.76-4.64 (m, 1H), 4.48 (t, J = 8.5 Hz, 1H), 4.35- 4.21 (m, 1H), 4.12 (t, J = 8.5 Hz, 1H), 4.04-3.94 (m, 1H), 3.92-3.85 (m, 1H), 3.84- 3.70 (m, 2H), 3.61- 3.47 (m, 1H), 3.10-2.89 (m, 2H), 2.85-2.69 (m, 1H), 2.42- 2.16 (m, 3H), 2.13- 2.03 (m, 3H), 2.02-1.90 (m, 6H), 1.87-1.41 (m, 11H), 1.30- 1.18 (m, 3H), 0.92- 0.56 (m, 7H), 0.52-0.36 (m, 2H); 958.5 [M + H]⁺ . |
| | C118 | 1H NMR (400 MHz, DMSO-d6) δ (ppm) 8.94-8.71 (m, 1H), 8.32- 8.22 (m, 1H), 8.11- 8.01 (m, 2H), 7.64-7.56 (m, 1H), 7.41-7.27 (m, 6H), 7.24- 7.10 (m, 4H), 6.30- 6.04 (m, 2H), 4.89-4.69 (m, 2H), 4.61-4.25 (m, 3H), 4.16- 4.08 (m, 1H), 3.84- 3.51 (m, 3H), 2.80-2.60 (m, 2H), 2.44-1.50 (m, 20H), 1.21- 1.02 (m, 8H), 0.96- 0.86 (m, 2H), 0.55-0.41 (m, 2H); 1011.2 [M + H]⁺ . |
| | C119 | 1H NMR (400 MHz, DMSO-d6) δ (ppm) 8.89-8.68 (m, 1H), 8.27 (d, J = 3.8 Hz, 1H), 8.13- 8.00 (m, 2H), 7.60 (t, J = 7.5 Hz, 1H), 7.40-7.26 (m, 6H), 7.24- 7.08 (m, 4H), 6.34- 6.04 (m, 2H), 4.77-4.62 (m, 1H), 4.57-4.41 (m, 2H), 4.34- 4.22 (m, 1H), 4.12 (t, J = 8.4 Hz, 1H), 3.96-3.66 (m, 4H), 3.60-3.47 (m, 1H), 3.11- 2.98 (m, 1H), 2.92- 2.79 (m, 1H), 2.60-2.54 (m, 1H), 2.47-2.40 (m, 1H), 2.34-1.41 (m, 21H), 1.17 (d, J = 7.2 Hz, 1H), 1.01- 0.92 (m, 4H), 0.81 (t, J = 7.4 Hz, 3H), 0.53-0.33 (m, 2H); 1024.8 [M + H]⁺ . |
| | C120 | 1H NMR (400 MHz, DMSO-d6) δ (ppm) 8.77 (d, J = 7.6 Hz, 1H), 8.26 (s, 1H), 8.09-8.00 (m, 2H), 7.59 (d, J = 8.5 Hz, 1H), 7.38- 7.27 (m, 6H), 7.24-7.11 (m, 4H), 6.31-6.13 (m, 2H), 4.85- 4.71 (m, 1H), 4.47 (t, J = 8.5 Hz, 1H), 4.36-4.25 (m, 1H), 4.16-4.07 (m, 1H), 3.95- 3.82 (m, 3H), 3.73 (t, J = 9.8 Hz, 1H), 3.58-3.48 (m, 1H), 3.03-2.86 (m, 1H), 2.47- 2.07 (m, 10H), 2.02- 1.44 (m, 15H), 1.17 (d, J = 7.1 Hz, 3H), 0.81 (t, J = 7.4 Hz, 3H), 0.53- 0.39 (m, 2H); 982.5 [M + H]⁺ . |
| | C121 | 1H NMR (400 MHz, DMSO-d6) δ (ppm) 10.01-9.50 (m, 1H), 8.95- 8.75 (m, 1H), 8.30- 8.25 (m, 1H), 8.12-8.04 (m, 2H), 7.62 (d, J = 8.5 Hz, 1H), 7.41 7.27 (m, 6H), 7.25- 7.12 (m, 4H), 6.31-6.02 (m, 2H), 4.88-4.48 (m, 5H), 4.21- 4.08 (m, 1H), 3.76- 3.73 (m, 1H), 3.67-3.60 (m, 1H), 3.57-3.50 (m, 1H), 3.19- 3.04 (m, 0.5H), 2.85-2.75 (m, 1H), 2.74-2.63 (m, 1.5H), 2.62- 2.57 (m, 3H), 2.57- 2.51 (m, 1H), 2.50-2.40 (m, 2H), 2.29-1.71 (m, 12H), 1.70- 1.47 (m, 2H), 1.15- 1.01 (m, 9H), 0.61 z-0.43 (m, 2H); 1010.6 [M+ H]+ |
| | C122 | 1H NMR (400 MHz, DMSO-d6) δ (ppm) 8.87-8.72 (m, 1H), 8.28- 8.19 (m, 1H), 8.08- 7.91 (m, 2H), 7.54-7.46 (m, 1H), 7.38-7.27 (m, 4H), 7.25- 7.16 (m, 1H), 6.07- 5.59 (m, 2H), 4.80-4.63 (m, 1H), 4.59-4.41 (m, 2H), 4.35- 4.23 (m, 1H), 4.17- 4.05 (m, 1H), 3.99-3.49 (m, 6H), 3.07-2.92 (m, 1H), 2.68- 2.62 (m, 1H), 2.45- 1.46 (m, 22H), 1.29-1.18 (m, 3H), 0.88-0.56 (m, 10H), 0.53- 0.35 (m, 2H); 989.2 [M + H]⁺ . |
| | C123 | 1H NMR (400 MHz, DMSO-d6) δ (ppm) 8.83-8.73 (m, 1H), 8.29- 8.19 (m, 1H), 8.06- 7.94 (m, 2H), 7.54-7.46 (m, 1H), 7.36-7.28 (m, 4H), 7.24- 7.16 (m, 1H), 6.07- 5.85 (m, 1H), 5.70-5.49 (m, 1H), 4.92-4.66 (m, 2H), 4.57- 4.44 (m, 2H), 4.30 (d, J = 5.3 Hz, 1H), 4.15-3.94 (m, 3H), 3.76-3.49 (m, 3H), 3.06- 2.93 (m, 1H), 2.65 (s, 2H), 2.45-2.12 (m, 7H), 2.03- 1.48 (m, 19H), 1.26- 1.15 (m, 6H), 0.53-0.39 (m, 2H); 974.6 [M + H]⁺ . |
| | C124 | 1H NMR (400 MHz, DMSO-d6) δ (ppm) 8.88-8.72 (m, 1H), 8.32- 8.21 (m, 1H), 8.11- 8.01 (m, 2H), 7.65-7.53 (m, 1H), 7.45-7.25 (m, 6H), 7.25- 7.06 (m, 4H), 6.38- 5.99 (m, 2H), 4.94-4.79 (m, 1H), 4.77-4.63 (m, 1H), 4.60- 4.39 (m, 2H), 4.35- 4.21 (m, 1H), 4.17-4.04 (m, 1H), 3.83-3.65 (m, 2H), 3.60- 3.44 (m, 1H), 3.08- 2.92 (m, 1H), 2.73-2.56 (m, 2H), 2.47-2.30 (m, 2H), 2.27- 2.09 (m, 5H), 2.01- 1.50 (m, 19H), 1.16-1.06 (m, 3H), 0.57-0.34 (m, 2H); 1022.4 [M + H]⁺ . |
| | C125 | 1H NMR (400 MHz, DMSO-d6) δ (ppm) 8.80 (d, J = 7.7 Hz, 1H), 8.27 (s, 1H), 8.11-8.01 (m, 2H), 7.61 (d, J = 8.6 Hz, 1H), 7.41- 7.10 (m, 10H), 6.26-6.05 (m, 2H), 4.80-4.63 (m, 2H), 4.58- 4.42 (m, 2H), 4.35- 4.23 (m, 1H), 4.19-4.06 (m, 1H), 3.85-3.69 (m, 2H), 3.61- 3.47 (m, 1H), 3.06- 2.93 (m, 1H), 2.72-2.58 (m, 2H), 2.47-2.29 (m, 2H), 2.28- 2.07 (m, 5H), 2.03- 1.92 (m, 6H), 1.91-1.75 (m, 5H), 1.74-1.44 (m, 8H), 1.12 (d, J = 7.1 Hz, 3H), 0.57- 0.37 (m, 2H); 1022.5 [M + H]⁺ . |
| | C126 | 1H NMR (400 MHz, DMSO-d6) δ (ppm) 8.86-8.78 (m, 1H), 8.29 (s, 1H), 8.12-8.04 (m, 2H), 7.65- 7.58 (m, 1H), 7.41- 7.30 (m, 6H), 7.26-7.14 (m, 4H), 6.32-6.04 (m, 2H), 4.79- 4.68 (m, 1H), 4.60- 4.45 (m, 2H), 4.37-4.26 (m, 1H), 4.19-4.09 (m, 1H), 4.04- 3.68 (m, 4H), 3.62- 3.49 (m, 1H), 3.42-3.37 (m, 1H), 3.20-3.10 (m, 3H), 3.07- 2.98 (m, 1H), 2.73- 2.61 (m, 2H), 2.47-2.32 (m, 3H), 2.30-2.21 (m, 1H), 2.20- 2.11 (m, 2H), 2.04- 1.95 (m, 6H), 1.91-1.32 (m, 11H), 0.89-0.71 (m, 3H), 0.57- 0.41 (m, 2H); 1041.2 [M + H]⁺ . |
| | C127 | 1H NMR (400 MHz, DMSO-d6) δ (ppm) 8.76 (d, J = 7.7 Hz, 1H), 8.26 (s, 1H), 8.11-7.98 (m, 2H), 7.62- 7.53 (m, 1H), 7.37- 7.26 (m, 6H), 7.24-7.12 (m, 4H), 6.21-5.97 (m, 1H), 5.95- 5.78 (m, 1H), 4.71 (t, J = 8.6 Hz, 1H), 4.57-4.41 (m, 2H), 4.37-4.21 (m, 1H), 4.12 (t, J = 8.6 Hz, 1H), 3.95- 3.81 (m, 2H), 3.73 (t, J = 9.9 Hz, 1H), 3.60-3.47 (m, 1H), 3.06- 2.95 (m, 1H), 2.72- 2.58 (m, 2H), 2.45-2.10 (m, 5H), 2.05-1.40 (m, 18H), 1.26- 1.17 (m, 5H), 0.84- 0.77 (m, 3H), 0.55-0.38 (m, 2H); 1024.6 [M+ H]+ |
| | C128 | 1H NMR (400 MHz, DMSO-d6) δ (ppm) 8.89-8.69 (m, 1H), 8.25 (s, 1H), 8.13-7.92 (m, 2H), 7.60- 7.45 (m, 1H), 7.39- 7.12 (m, 11H), 7.11-6.92 (m, 4H), 6.40-5.95 (m, 2H), 4.77- 4.66 (m, 1H), 4.59- 4.42 (m, 2H), 4.35-4.23 (m, 1H), 4.18-4.07 (m, 1H), 3.99- 3.86 (m, 1H), 3.85- 3.78 (m, 1H), 3.76-3.46 (m, 3H), 3.06-2.94 (m, 1H), 2.91- 2.77 (m, 1H), 2.75- 2.63 (m, 2H), 2.44-2.28 (m, 2H), 2.27-2.06 (m, 3H), 1.92- 1.17 (m, 18H), 0.74- 0.58 (m, 3H), 0.53-0.37 (m, 2H); 1086.9 [M + H]⁺ . |
| | C129 | 1H NMR (400 MHz, DMSO-d6) δ (ppm) 8.82-8.70 (m, 1H), 8.33- 8.21 (m, 1H), 8.10- 8.01 (m, 2H), 7.65-7.55 (m, 1H), 7.37-7.28 (m, 6H), 7.24- 7.14 (m, 4H), 6.26- 6.05 (m, 2H), 4.83-4.66 (m, 1H), 4.52-4.40 (m, 1H), 4.34- 4.22 (m, 1H), 4.17- 4.06 (m, 1H), 3.84-3.63 (m, 3H), 3.60-3.47 (m, 2H), 3.05- 2.89 (m, 2H), 2.47- 2.16 (m, 8H), 2.12-2.07 (m, 3H), 2.04-1.93 (m, 3H), 1.86- 1.53 (m, 11H), 1.18- 1.07 (m, 3H), 0.84-0.71 (m, 6H), 0.55-0.44 (m, 2H); 1023.0 [M + H]⁺ . |
| | C130 | 1H NMR (400 MHz, DMSO-d6) δ (ppm) 8.80-8.69 (m, 1H), 8.27 (s, 1H), 8.09-8.00 (m, 2H), 7.63- 7.54 (m, 1H), 7.39- 7.26 (m, 6H), 7.24-7.12 (m, 4H), 6.27-5.95 (m, 3H), 4.80- 4.68 (m, 1H), 4.47 (t, J = 8.5 Hz, 1H), 4.34-4.23 (m, 1H), 4.11 (t, J = 8.6 Hz, 1H), 3.83- 3.46 (m, 6H), 3.00- 2.90 (m, 1H), 2.70-2.59 (m, 1H), 2.38-2.31 (m, 2H), 2.26- 2.18 (m, 1H), 2.15- 2.07 (m, 4H), 2.05-1.92 (m, 5H), 1.85-1.63 (m, 10H), 1.60- 1.52 (m, 2H), 1.19- 1.07 (m, 3H), 0.86-0.71 (m, 6H), 0.54-0.40 (m, 2H); 1004.7 [M + H]⁺ . |
| | C131 | 1H NMR (400 MHz, DMSO-d6) δ (ppm) 8.81-8.71 (m, 1H), 8.31- 8.21 (m, 1H), 8.10- 8.01 (m, 2H), 7.65-7.55 (m, 1H), 7.41-7.26 (m, 6H), 7.25- 7.09 (m, 4H), 6.29- 6.04 (m, 2H), 4.84-4.68 (m, 1H), 4.54-4.41 (m, 1H), 4.36- 4.21 (m, 1H), 4.16- 4.06 (m, 1H), 3.89-3.62 (m, 3H), 3.61-3.48 (m, 2H), 3.00- 2.86 (m, 1H), 2.41- 2.29 (m, 2H), 2.27-2.17 (m, 1H), 2.15-2.06 (m, 4H), 2.05- 1.47 (m, 15H), 1.17- 1.08 (m, 3H), 0.90-0.83 (m, 2H), 0.81-0.70 (m, 8H), 0.56- 0.36 (m, 2H); 1022.4 [M + H]⁺ . |
| | C132 | 1H NMR (400 MHz, DMSO-d6) δ (ppm) 8.82-8.68 (m, 1H), 8.26 (s, 1H), 8.12-7.95 (m, 2H), 7.60 (d, J = 8.0 Hz, 1H), 7.35 (t, J = 7.9 Hz, 2H), 7.17 (t, J = 8.7 Hz, 3H), 6.29-6.18 (m, 1H), 6.15- 6.05 (m, 1H), 4.70 (t, J = 8.5 Hz, 1H), 4.55-4.39 (m, 2H), 4.36-4.20 (m, 1H), 3.90- 3.53 (m, 5H), 3.03- 2.90 (m, 1H), 2.70-2.56 (m, 2H), 2.42-2.34 (m, 1H), 2.31- 2.24 (m, 1H), 2.14- 2.05 (m, 1H), 1.99-1.51 (m, 20H), 1.20-1.08 (m, 3H), 0.88- 0.75 (m, 6H), 0.44- 0.29 (m, 2H); 948.4 [M + H]⁺ . |
| | C133 | 1H NMR (400 MHz, DMSO-d6) δ (ppm) 8.81-8.70 (m, 1H), 8.27 (s, 1H), 8.13-7.99 (m, 2H), 7.60 (d, J = 7.7 Hz, 1H), 7.41- 7.10 (m, 10H), 6.30-6.04 (m, 2H), 4.86-4.67 (m, 1H), 4.55- 4.41 (m, 1H), 4.37- 4.23 (m, 1H), 4.20-4.02 (m, 3H), 3.86-3.49 (m, 5H), 3.03- 2.86 (m, 1H), 2.48- 1.48 (m, 22H), 1.21-1.07 (m, 3H), 0.86-0.74 (m, 6H), 0.57- 0.38 (m, 2H); 1012.5 [M + H]⁺ . |
| | C134 | 1H NMR (400 MHz, DMSO-d6) δ (ppm) 8.78 (d, J = 7.4 Hz, 1H), 8.27 (s, 1H), 8.09-8.00 (m, 2H), 7.60 (d, J = 8.7 Hz, 1H), 7.40- 7.25 (m, 6H), 7.23-7.12 (m, 4H), 6.27-6.03 (m, 2H), 4.76- 4.62 (m, 1H), 4.47 (t, J = 8.6 Hz, 1H), 4.35-4.23 (m, 1H), 4.12 (t, J = 8.8 Hz, 1H), 3.89- 3.62 (m, 3H), 3.60- 3.48 (m, 2H), 3.06-2.92 (m, 2H), 2.87-2.70 (m, 1H), 2.37- 2.15 (m, 3H), 2.15- 1.91 (m, 9H), 1.85-1.45 (m, 10H), 1.13 (d, J = 7.1 Hz, 3H), 0.77 (d, J = 6.7 Hz, 6H), 0.55-0.36 (m, 2H); 1008.4 [M + H]⁺ . |
| | C135 | 1H NMR (400 MHz, DMSO-d6) δ (ppm) 8.85-8.67 (m, 1H), 8.34- 8.18 (m, 1H), 8.12- 7.99 (m, 2H), 7.68-7.53 (m, 1H), 7.39-7.14 (m, 10H), 6.32- 6.07 (m, 2H), 4.89- 4.69 (m, 1H), 4.56-4.40 (m, 1H), 4.36-4.21 (m, 1H), 4.16- 4.04 (m, 1H), 3.85- 3.67 (m, 4H), 3.58-3.44 (m, 1H), 3.01-2.85 (m, 1H), 2.46- 2.17 (m, 7H), 2.14- 2.08 (m, 3H), 2.05-1.95 (m, 3H), 1.87-1.52 (m, 11H), 1.19- 1.07 (m, 3H), 0.89- 0.75 (m, 6H), 0.54-0.37 (m, 2H); 996.4 [M + H]⁺ . |
| | C136 | 1H NMR (400 MHz, DMSO-d6) δ (ppm) 8.77 (d, J = 7.8 Hz, 1H), 8.27 (s, 1H), 8.10-8.00 (m, 2H), 7.60 (d, J = 8.4 Hz, 1H), 7.40- 7.25 (m, 6H), 7.24-7.11 (m, 4H), 6.27-6.02 (m, 2H), 4.86- 4.72 (m, 1H), 4.47 (t, J = 8.6 Hz, 1H), 4.36-4.25 (m, 1H), 4.16-4.05 (m, 1H), 3.90- 3.45 (m, 5H), 3.01- 2.87 (m, 1H), 2.44-2.17 (m, 6H), 2.12 (s, 3H), 2.10-1.90 (m, 4H), 1.87-1.51 (m, 11H), 1.13 (d, J = 7.1 Hz, 3H), 0.77 (d, J = 6.7 Hz, 6H), 0.53-0.40 (m, 2H); 996.5 [M + H]⁺ . |
| | C137 | 1H NMR (400 MHz, DMSO-d6) δ (ppm) 8.82-8.74 (m, 1H), 8.28 (s, 1H), 8.10-8.01 (m, 2H), 7.64- 7.55 (m, 1H), 7.40- 7.27 (m, 6H), 7.24-7.13 (m, 4H), 6.27-6.05 (m, 2H), 4.80- 4.72 (m, 1H), 4.50- 4.41 (m, 1H), 4.36-4.27 (m, 1H), 4.14-4.07 (m, 1H), 3.97- 3.67 (m, 5H), 3.58- 3.46 (m, 4H), 3.01-2.91 (m, 1H), 2.46-2.31 (m, 2H), 2.26- 1.64 (m, 16H), 1.60- 1.37 (m, 5H), 1.16-1.09 (m, 3H), 0.87-0.74 (m, 3H), 0.52- 0.39 (m, 2H); 996.5 [M + H]⁺ . |
| | C138 | 1H NMR (400 MHz, DMSO-d6) δ (ppm) 8.80-8.71 (m, 1H), 8.26 (s, 1H), 8.11-8.00 (m, 2H), 7.60 (d, J = 8.5 Hz, 1H), 7.39- 7.27 (m, 6H), 7.23-7.12 (m, 4H), 6.25-5.93 (m, 3H), 4.82- 4.69 (m, 1H), 4.47 (t, J = 8.4 Hz, 1H), 4.35-4.23 (m, 1H), 4.11 (t, J = 8.6 Hz, 1H), 3.97- 3.67 (m, 4H), 3.59- 3.46 (m, 1H), 3.01-2.79 (m, 1H), 2.75-2.57 (m, 1H), 2.37- 1.34 (m, 26H), 1.13 (d, J = 7.1 Hz, 3H), 0.84-0.75 (m, 3H), 0.54-0.43 (m, 2H); 990.4 [M + H]⁺ . |
| | C139 | 1H NMR (400 MHz, DMSO-d6) δ (ppm) 8.80-8.72 (m, 1H), 8.27 (s, 1H), 8.09-8.02 (m, 2H), 7.64- 7.56 (m, 1H), 7.41- 7.26 (m, 6H), 7.25-7.11 (m, 4H), 6.29-6.05 (m, 2H), 4.83- 4.68 (m, 1H), 4.51- 4.39 (m, 1H), 4.35-4.24 (m, 1H), 4.15-4.06 (m, 1H), 3.99- 3.87 (m, 1H), 3.87- 3.67 (m, 3H), 3.60-3.47 (m, 1H), 3.02-2.87 (m, 1H), 2.61- 2.51 (m, 1H), 2.42- 2.30 (m, 2H), 2.27-2.17 (m, 1H), 2.15-2.06 (m, 4H), 2.03- 1.91 (m, 3H), 1.89- 1.62 (m, 8H), 1.60-1.47 (m, 3H), 1.47-1.36 (m, 1H), 1.17- 1.09 (m, 3H), 0.90- 0.71 (m, 7H), 0.56-0.40 (m, 2H); 1008.6 [M + H]⁺ . |
| | C140 | 1H NMR (400 MHz, DMSO-d6) δ (ppm) 8.82-8.69 (m, 1H), 8.27 (s, 1H), 8.12-8.00 (m, 2H), 7.61 (d, J = 8.6 Hz, 1H), 7.40- 7.12 (m, 10H), 6.31-5.99 (m, 2H), 4.87-4.66 (m, 1H), 4.55- 4.40 (m, 1H), 4.37- 4.23 (m, 1H), 4.20-3.99 (m, 3H), 3.92-3.64 (m, 4H), 3.58- 3.47 (m, 1H), 3.02- 2.89 (m, 1H), 2.49-2.43 (m, 1H), 2.41-2.30 (m, 2H), 2.26- 2.18 (m, 1H), 2.17- 2.06 (m, 4H), 2.05-1.93 (m, 3H), 1.89-1.51 (m, 10H), 1.47- 1.36 (m, 2H), 1.13 (d, J = 7.1 Hz, 3H), 0.83-0.71 (m, 3H), 0.62-0.33 (m, 2H); 998.3 [M + H]⁺ . |
| | C141 | 1H NMR (400 MHz, DMSO-d6) δ (ppm) 8.86-8.66 (m, 1H), 8.27 (s, 1H), 8.17-7.95 (m, 2H), 7.60 (d, J = 8.3 Hz, 1H), 7.40- 7.26 (m, 6H), 7.24-7.14 (m, 4H), 6.39-6.05 (m, 2H), 4.84- 4.69 (m, 1H), 4.58- 4.40 (m, 1H), 4.37-4.24 (m, 1H), 4.22-4.01 (m, 3H), 4.01- 3.85 (m, 2H), 3.81- 3.65 (m, 2H), 3.64-3.42 (m, 1H), 3.05-2.83 (m, 1H), 2.48- 2.41 (m, 1H), 2.41- 2.30 (m, 2H), 2.27-2.17 (m, 1H), 2.16-2.05 (m, 4H), 2.04- 1.92 (m, 3H), 1.90- 1.62 (m, 8H), 1.60-1.46 (m, 4H), 1.13 (d, J = 7.0 Hz, 3H), 0.83 (t, J = 7.4 Hz, 3H), 0.58-0.39 (m, 2H); 998.3 [M + H]⁺ . |
| | C142 | 1H NMR (400 MHz, DMSO-d6) δ (ppm) 8.82-8.75 (m, 1H), 8.26 (s, 1H), 8.09-8.03 (m, 2H), 7.60 (d, J = 7.3 Hz, 1H), 7.40- 7.26 (m, 6H), 7.22-7.11 (m, 4H), 6.27-6.18 (m, 1H), 6.15- 6.05 (m, 1H), 4.76- 4.68 (m, 1H), 4.48 (t, J = 8.4 Hz, 1H), 4.34-4.24 (m, 1H), 4.12 (t, J = 8.6 Hz, 1H), 3.97- 3.90 (m, 1H), 3.85-3.70 (m, 3H), 3.59-3.49 (m, 1H), 3.12- 2.93 (m, 2H), 2.39- 2.32 (m, 1H), 2.26-2.18 (m, 2H), 2.16-1.93 (m, 11H), 1.88- 1.81 (m, 1H), 1.76- 1.37 (m, 10H), 1.13 (d, J = 7.0 Hz, 3H), 0.85-0.75 (m, 3H), 0.53- 0.41 (m, 2H); 976.5 [M + H]⁺ . |
| | C143 | 1H NMR (400 MHz, DMSO-d6) δ (ppm) 8.96-8.69 (m, 1H), 8.39- 8.19 (m, 1H), 8.14- 8.00 (m, 2H), 7.67-7.55 (m, 1H), 7.41-7.28 (m, 6H), 7.24- 7.10 (m, 4H), 6.30- 6.04 (m, 2H), 4.90-4.73 (m, 1H), 4.63-4.42 (m, 2H), 4.21- 4.06 (m, 1H), 3.99- 3.85 (m, 2H), 3.78-3.71 (m, 2H), 3.58-3.52 (m, 3H), 2.75- 2.60 (m, 3H), 2.48- 2.40 (m, 1H), 2.40-2.15 (m, 5H), 2.15-1.59 (m, 13H), 1.59- 1.30 (m, 3H), 1.20- 1.06 (m, 3H), 0.87-0.72 (m, 3H), 0.60-0.37 (m, 2H); 976.6 [M + H]⁺ . |
| | C144 | 1H NMR (400 MHz, DMSO-d6) δ (ppm) 8.84-8.73 (m, 1H), 8.31- 8.23 (m, 1H), 8.11- 8.01 (m, 2H), 7.65-7.55 (m, 1H), 7.39-7.12 (m, 10H), 6.31- 6.03 (m, 2H), 4.84- 4.72 (m, 1H), 4.53-4.41 (m, 1H), 4.39-4.27 (m, 1H), 4.16- 4.05 (m, 1H), 3.99- 3.68 (m, 4H), 3.58-3.49 (m, 1H), 3.16-2.84 (m, 1H), 2.47- 1.31 (m, 25H), 1.19- 1.06 (m, 3H), 0.87-0.73 (m, 3H), 0.54-0.38 (m, 2H); 983.6 [M + H]⁺ . |
| | C145 | 1H NMR (400 MHz, DMSO-d6) δ (ppm) 8.74 (d, J = 7.2 Hz, 1H), 8.28 (s, 1H), 8.11-8.02 (m, 2H), 7.60 (d, J = 8.5 Hz, 1H), 7.41 7.30 (m, 6H), 7.24- 7.16 (m, 4H), 6.28- 6.11 (m, 2H), 4.99- 4.91 (m, 1H), 4.49 (t, J = 8.2 Hz, 1H), 4.37-4.25 (m, 1H), 4.19-4.08 (m, 1H), 4.00-3.88 (m, 2H), 3.82-3.72 (m, 2H), 3.64-3.50 (m, 5H), 2.85-2.72 (m, 1H), 2.44-2.33 (m, 3H), 2.26-2.20 (m, 1H), 2.12-1.50 (m, 16H), 1.13 (d, J = 7.0 Hz, 3H), 0.83 (t, J = 7.4 Hz, 3H), 0.56-0.40 (m, 2H); 928.6 [M + H]⁺ . |
| | C146 | 1H NMR (400 MHz, DMSO-d6) δ (ppm) 8.83-8.69 (m, 1H), 8.27 (s, 1H), 8.13-7.97 (m, 2H), 7.67- 7.50 (m, 1H), 7.44- 7.05 (m, 10H), 6.30-6.01 (m, 2H), 4.81-4.69 (m, 1H), 4.55- 4.04 (m, 7H), 3.98- 3.66 (m, 4H), 3.56-3.50 (m, 1H), 2.88-2.79 (m, 1H), 2.40- 2.31 (m, 1H), 2.24- 2.17 (m, 1H), 2.12-1.35 (m, 18H), 1.28 (s, 3H), 1.26-1.20 (m, 1H), 1.18-1.07 (m, 3H), 0.85- 0.74 (m, 3H), 0.56- 0.35 (m, 2H); 956.5 [M + H]⁺ . |
| | C147 | 1H NMR (400 MHz, DMSO-d6) δ (ppm) 8.81-8.74 (m, 1H), 8.27 (s, 1H), 8.10-8.02 (m, 2H), 7.65- 7.56 (m, 1H), 7.40- 7.26 (m, 6H), 7.24-7.12 (m, 4H), 6.28-6.05 (m, 2H), 4.78- 4.64 (m, 1H), 4.59- 4.50 (m, 2H), 4.49-4.38 (m, 2H), 4.38-4.33 (m, 1H), 4.33- 4.24 (m, 1H), 4.17- 4.06 (m, 1H), 3.99-3.86 (m, 1H), 3.85-3.68 (m, 4H), 3.59- 3.49 (m, 1H), 2.89- 2.76 (m, 1H), 2.40-2.30 (m, 1H), 2.27-2.16 (m, 1H), 2.15- 2.07 (m, 1H), 2.04- 1.99 (m, 1H), 1.96 (s, 4H), 1.86- 1.68 (m, 3H), 1.65- 1.55 (m, 3H), 1.54-1.37 (m, 3H), 1.23 (s, 2H), 1.18-1.08 (m, 3H), 0.87-0.82 (m, 1H), 0.81- 0.74 (m, 2H), 0.57- 0.42 (m, 2H); 928.6 [M + H]⁺ . |
| | C148 | 1H NMR (400 MHz, DMSO-d6) δ (ppm) 8.93-8.74 (m, 1H), 8.32- 8.23 (m, 1H), 8.11- 8.02 (m, 2H), 7.65-7.57 (m, 1H), 7.38-7.28 (m, 6H), 7.24- 7.14 (m, 4H), 6.39- 6.04 (m, 3H), 4.87-4.76 (m, 1H), 4.61-4.50 (m, 2H), 4.18- 4.10 (m, 1H), 3.79- 3.72 (m, 3H), 3.58-3.54 (m, 2H), 3.39-3.06 (m, 3H), 2.83- 2.68 (m, 3H), 2.45- 1.64 (m, 16H), 1.62-1.50 (m, 1H), 1.18-1.10 (m, 3H), 0.84- 0.76 (m, 6H), 0.58- 0.44 (m, 2H); 964.9 [M + H]⁺ . |
| | C149 | 1H NMR (400 MHz, DMSO-d6) δ (ppm) 8.83-8.64 (m, 1H), 8.30- 8.21 (m, 1H), 8.14- 7.93 (m, 2H), 7.64-7.52 (m, 1H), 7.42-7.26 (m, 6H), 7.24- 7.07 (m, 4H), 6.53- 5.89 (m, 3H), 4.84-4.67 (m, 1H), 4.53-4.43 (m, 1H), 4.37- 4.26 (m, 1H), 4.19- 4.06 (m, 1H), 4.03-3.63 (m, 4H), 3.60-3.45 (m, 1H), 3.43- 3.35 (m, 1H), 3.26- 3.18 (m, 1H), 3.18-3.06 (m, 3H), 3.02-2.97 (m, 1H), 2.47- 2.31 (m, 3H), 2.26- 2.18 (m, 1H), 2.17-2.07 (m, 4H), 2.05-1.92 (m, 5H), 1.89- 1.62 (m, 6H), 1.61- 1.41 (m, 3H), 1.40-1.32 (m, 1H), 1.29-1.18 (m, 1H), 0.89- 0.70 (m, 3H), 0.54- 0.37 (m, 2H); 980.9 [M + H]⁺ . |
| | C150 | 1H NMR (400 MHz, DMSO-d6) δ (ppm) 8.73 (d, J = 7.5 Hz, 1H), 8.26 (s, 1H), 8.10-8.00 (m, 2H), 7.63- 7.54 (m, 1H), 7.37- 7.27 (m, 6H), 7.24-7.12 (m, 4H), 6.24-5.83 (m, 3H), 4.84- 4.69 (m, 1H), 4.52- 4.43 (m, 1H), 4.37-4.25 (m, 1H), 4.16-4.05 (m, 1H), 3.95- 3.83 (m, 2H), 3.78- 3.67 (m, 1H), 3.59-3.47 (m, 1H), 3.06-2.92 (m, 1H), 2.45- 2.31 (m, 2H), 2.29- 1.40 (m, 21H), 1.32-1.20 (m, 6H), 0.84-0.78 (m, 3H), 0.55- 0.38 (m, 2H); 964.6 [M + H]⁺ . |
| | C151 | 1H NMR (400 MHz, DMSO-d6) δ (ppm) 8.83-8.72 (m, 1H), 8.26 (s, 1H), 8.11-8.01 (m, 2H), 7.60 (d, J = 8.5 Hz, 1H), 7.38- 7.27 (m, 6H), 7.24-7.12 (m, 4H), 6.24-5.93 (m, 3H), 4.89- 4.66 (m, 2H), 4.54- 4.44 (m, 1H), 4.38-4.26 (m, 1H), 4.16-4.06 (m, 1H), 3.76- 3.50 (m, 3H), 3.31- 3.28 (m, 1H), 3.05-2.91 (m, 1H), 2.45-2.32 (m, 2H), 2.24- 1.37 (m, 21H), 1.15- 1.12 (m, 3H), 1.08 (d, J = 6.2 Hz, 1H), 0.96 (d, J = 6.2 Hz, 1H), 0.87- 0.73 (m, 2H), 0.65- 0.60 (m, 1H), 0.53-0.39 (m, 2H); 964.6 [M + H]⁺ . |
| | C152 | 1H NMR (400 MHz, DMSO-d6) δ (ppm) 10.02-9.48 (m, 1H), 9.02- 8.80 (m, 1H), 8.32 (s, 1H), 8.27-8.18 (m, 2H), 7.73- 7.65 (m, 1H), 7.45- 7.15 (m, 9H), 7.10 (d, J = 8.2 Hz, 1H), 4.87-4.76 (m, 1H), 4.75- 4.46 (m, 3H), 4.38- 4.15 (m, 1H), 3.97-3.59 (m, 6H), 3.33-3.02 (m, 3H), 2.85- 2.54 (m, 6H), 2.47- 2.42 (m, 1H), 2.32-1.42 (m, 20H), 0.81 (t, J = 7.4 Hz, 3H), 0.63- 0.41 (m, 2H); 1055.0 [M + H]⁺ . |
| | C153 | 1H NMR (400 MHz, DMSO-d6) δ (ppm) 9.75-9.45 (m, 1H), 8.98- 8.71 (m, 1H), 8.30- 8.20 (m, 1H), 8.12-8.05 (m, 1H), 8.05-7.99 (m, 1H), 7.59- 7.48 (m, 1H), 7.39- 7.27 (m, 4H), 7.25-7.19 (m, 1H), 6.41-6.19 (m, 1H), 4.87- 4.74 (m, 1H), 4.68- 4.48 (m, 3H), 4.19-4.05 (m, 4H), 4.03-3.89 (m, 1H), 3.81- 3.52 (m, 5H), 3.21- 3.10 (m, 1H), 2.85-2.75 (m, 1H), 2.68-2.56 (m, 4H), 2.43- 1.75 (m, 14H), 1.65- 1.44 (m, 4H), 1.32-1.15 (m, 6H), 0.91-0.84 (m, 3H), 0.61- 0.41 (m, 2H); 1025.1 [M+ H]+ |
| | C154 | 1H NMR (400 MHz, DMSO-d6) δ (ppm) 9.72-9.45 (m, 1H), 9.02- 8.78 (m, 1H), 8.36- 8.27 (m, 1H), 8.23-8.14 (m, 2H), 7.67 (d, J = 8.7 Hz, 1H), 7.39- 7.30 (m, 6H), 7.24- 7.11 (m, 4H), 6.86-6.75 (m, 1H), 4.89-4.75 (m, 1H), 4.73- 4.46 (m, 3H), 4.22- 4.07 (m, 1H), 3.97-3.72 (m, 4H), 3.66-3.49 (m, 2H), 2.85- 2.55 (m, 6H), 2.46- 2.41 (m, 1H), 2.25-1.77 (m, 11H), 1.66-1.14 (m, 9H), 0.86- 0.78 (m, 3H), 0.62- 0.41 (m, 2H); 1028.7 [M + H]⁺ . |
| | C155 | 1H NMR (400 MHz, DMSO-d6) δ (ppm) 8.80 (d, J = 7.8 Hz, 1H), 8.24 (s, 1H), 8.04-7.91 (m, 2H), 7.51 (d, J = 8.5 Hz, 1H), 7.39- 7.26 (m, 4H), 7.24-7.16 (m, 1H), 5.99-5.76 (m, 1H), 5.05- 4.93 (m, 1H), 4.89- 4.79 (m, 1H), 4.75-4.66 (m, 1H), 4.58-4.43 (m, 2H), 4.34- 4.24 (m, 1H), 4.19- 4.08 (m, 1H), 4.01-3.81 (m, 4H), 3.78-3.70 (m, 1H), 3.64- 3.49 (m, 2H), 3.06- 2.91 (m, 1H), 2.72-2.56 (m, 2H), 2.44-2.33 (m, 2H), 2.25- 2.10 (m, 3H), 2.03- 1.43 (m, 20H), 1.30-1.23 (m, 3H), 1.18 (d, J = 7.2 Hz, 3H), 0.90- 0.79 (m, 6H), 0.54- 0.39 (m, 2H); 1048.4 [M + H]⁺ . |
| | C156 | 1H NMR (400 MHz, DMSO-d6) δ (ppm) 8.87-8.70 (m, 1H), 8.32- 8.20 (m, 1H), 8.08- 7.98 (m, 1H), 7.98-7.92 (m, 1H), 7.55-7.44 (m, 1H), 7.38- 7.26 (m, 4H), 7.24- 7.17 (m, 1H), 6.11-5.60 (m, 2H), 4.78-4.64 (m, 1H), 4.59- 4.42 (m, 2H), 4.36- 4.23 (m, 1H), 4.17-4.06 (m, 1H), 4.02-3.86 (m, 1H), 3.86- 3.60 (m, 4H), 3.59- 3.46 (m, 1H), 3.08-2.92 (m, 1H), 2.74-2.57 (m, 2H), 2.47- 2.29 (m, 2H), 2.28- 2.18 (m, 1H), 2.17-2.08 (m, 2H), 2.05-1.92 (m, 6H), 1.90- 1.45 (m, 10H), 1.32- 0.97 (m, 3H), 0.89-0.82 (m, 6H), 0.81-0.62 (m, 2H), 0.61- 0.53 (m, 2H), 0.54- 0.35 (m, 2H); 988.4 [M + H]⁺ . |
| | C157 | 1H NMR (400 MHz, DMSO-d6) δ (ppm) 9.68-9.40 (m, 1H), 8.96- 8.66 (m, 1H), 8.34- 8.24 (m, 1H), 8.10-7.98 (m, 2H), 7.63-7.50 (m, 1H), 7.40- 7.25 (m, 6H), 7.25- 7.11 (m, 4H), 6.67-6.52 (m, 1H), 6.26-6.03 (m, 1H), 5.05- 4.76 (m, 2H), 4.65- 4.45 (m, 2H), 4.25-4.06 (m, 1H), 3.90-3.72 (m, 5H), 3.68- 3.59 (m, 3H), 2.85- 2.53 (m, 6H), 2.45-1.34 (m, 17H), 0.97-0.85 (m, 1H), 0.85- 0.75 (m, 3H), 0.75- 0.44 (m, 3H); 957.4 [M + H]⁺ . |
| | C158 | 1H NMR (400 MHz, DMSO-d6) δ (ppm) 8.95-8.68 (m, 1H), 8.27 (s, 1H), 8.10-7.98 (m, 2H), 7.61 (d, J = 8.5 Hz, 1H), 7.41- 7.25 (m, 6H), 7.23-7.09 (m, 4H), 6.27-6.02 (m, 2H), 4.95- 4.63 (m, 2H), 4.60- 4.27 (m, 2H), 4.19-4.04 (m, 1H), 3.80-3.52 (m, 5H), 2.78- 2.57 (m, 2H), 2.40- 1.37 (m, 24H), 1.17-1.08 (m, 3H), 0.84-0.71 (m, 6H), 0.55- 0.38 (m, 2H); 1038.5 [M + H]⁺ . |
| | C159 | 1H NMR (400 MHz, DMSO-d6) δ (ppm) 8.85-8.69 (m, 1H), 8.32- 8.20 (m, 1H), 8.13- 8.00 (m, 2H), 7.66-7.54 (m, 1H), 7.42-7.25 (m, 6H), 7.24- 7.10 (m, 4H), 6.31- 6.06 (m, 2H), 4.84-4.66 (m, 2H), 4.54-4.40 (m, 1H), 4.35- 4.20 (m, 1H), 4.18- 4.05 (m, 1H), 3.92-3.66 (m, 4H), 3.58-3.48 (m, 1H), 3.19- 2.97 (m, 1H), 2.91- 2.72 (m, 1H), 2.64-2.52 (m, 1H), 2.39-2.30 (m, 1H), 2.26- 2.18 (m, 1H), 2.16- 1.47 (m, 21H), 1.21-1.15 (m, 1H), 1.02-0.92 (m, 2H), 0.85- 0.76 (m, 6H), 0.56- 0.32 (m, 2H); 1038.3 [M + H]⁺ . |
| | C160 | 1H NMR (400 MHz, DMSO-d6) δ (ppm) 8.82-8.72 (m, 1H), 8.29- 8.23 (m, 1H), 8.10- 8.01 (m, 2H), 7.60 (t, J = 7.7 Hz, 1H), 7.40-7.27 (m, 6H), 7.24- 7.10 (m, 4H), 6.30- 6.06 (m, 2H), 4.82-4.68 (m, 2H), 4.47 (t, J = 8.6 Hz, 1H), 4.33- 4.23 (m, 1H), 4.11 (t, J = 8.6 Hz, 1H), 3.97-3.65 (m, 4H), 3.58-3.46 (m, 1H), 3.13- 3.01 (m, 1H), 2.87- 2.76 (m, 1H), 2.61-2.53 (m, 1H), 2.39-2.28 (m, 1H), 2.22 (t, J = 11.5 Hz, 1H), 2.16-2.03 (m, 4H), 2.02-1.93 (m, 3H), 1.91- 1.78 (m, 4H), 1.75- 1.43 (m, 11H), 1.20-0.91 (m, 3H), 0.81 (t, J = 7.4 Hz, 3H), 0.52- 0.39 (m, 2H); 1024.6 [M+ H]+ |
| | C161 | 1H NMR (400 MHz, DMSO) δ 8.87-8.72 (m, 1H), 8.35-8.20 (m, 1H), 8.12- 8.00 (m, 2H), 7.66- 7.55 (m, 1H), 7.42-7.27 (m, 6H), 7.25-7.07 (m, 4H), 6.32- 6.06 (m, 2H), 4.91- 4.63 (m, 2H), 4.54-4.41 (m, 1H), 4.54-4.40 (m, 1H), 4.35- 4.06 (m, 4H), 4.02- 3.68 (m, 2H), 3.63-3.38 (m, 1H), 3.09-2.90 (m, 1H), 2.64- 2.55 (m, 1H), 2.44- 1.91 (m, 16H), 1.88-1.45 (m, 11H), 1.22-0.95 (m, 3H), 0.59- 0.37 (m, 2H); 988.4 [M + H]⁺ . |
| | C162 | 1H NMR (400 MHz, DMSO-d6) δ (ppm) 8.81 (d, J = 7.6 Hz, 1H), 8.30- 8.21 (m, 1H), 8.03 (t, J = 8.1 Hz, 1H), 7.97 (s, 1H), 7.56- 7.46 (m, 1H), 7.38- 7.26 (m, 4H), 7.25-7.17 (m, 1H), 6.09-5.90 (m, 1H), 5.86- 5.69 (m, 1H), 4.91- 4.65 (m, 2H), 4.47 (t, J = 8.6 Hz, 1H), 4.33-4.07 (m, 4H), 4.04- 3.50 (m, 5H), 3.07- 2.93 (m, 1H), 2.73-2.56 (m, 3H), 2.46-1.41 (m, 23H), 1.30- 1.18 (m, 3H), 0.89- 0.78 (m, 3H), 0.60-0.36 (m, 2H); 965.0 [M + H]⁺ . |
| | C163 | 1H NMR (400 MHz, DMSO-d6) δ (ppm) 10.13-9.47 (m, 1H), 8.95-8.71 (m, 1H), 8.73-8.46 (m, 2H), 8.26 (s, 1H), 8.10-7.88 (m, 3H), 7.68-7.46 (m, 2H), 7.40-7.25 (m, 2H), 7.23-7.06 (m, 3H), 6.30-6.05 (m, 2H), 4.86-4.48 (m, 4H), 4.25-4.10 (m, 1H), 3.97-3.89 (m, 1H), 3.86-3.75 (m, 5H), 2.85-2.55 (m, 6H), 2.48-2.36 (m, 2H), 2.25-1.38 (m, 16H), 1.13 (d, J = 6.9 Hz, 3H), 0.86- 0.72 (m, 3H), 0.59- 0.43 (m, 2H); 1011.5 [M+ H]+ |
| | C164 | 1H NMR (400 MHz, DMSO-d6) δ (ppm) 9.97-9.42 (m, 1H), 8.98- 8.72 (m, 1H), 8.71- 8.51 (m, 2H), 8.35-8.21 (m, 1H), 8.14-7.88 (m, 3H), 7.65- 7.47 (m, 2H), 7.40- 7.28 (m, 2H), 7.22-7.09 (m, 3H), 6.31-6.05 (m, 2H), 4.87- 4.75 (m, 1H), 4.72- 4.34 (m, 4H), 3.97-3.90 (m, 1H), 3.85-3.77 (m, 3H), 3.64- 3.62 (m, 2H), 2.85- 2.53 (m, 6H), 2.48-2.26 (m, 3H), 2.23-1.36 (m, 15H), 1.14 (d, J = 7.1 Hz, 3H), 0.87- 0.74 (m, 3H), 0.60-0.43 (m, 2H); 1011.5 [M+ H]+ |
| | C165 | 1H NMR (400 MHz, DMSO-d6) δ (ppm) 9.69-9.43 (m, 1H), 8.95- 8.68 (m, 1H), 8.32- 8.23 (m, 1H), 8.13-8.01 (m, 2H), 7.67-7.56 (m, 1H), 7.39- 7.27 (m, 6H), 7.27- 7.13 (m, 4H), 6.26-6.05 (m, 2H), 4.87-4.77 (m, 1H), 4.73- 4.48 (m, 3H), 4.42- 4.23 (m, 1H), 3.98-3.74 (m, 4H), 3.70-3.56 (m, 2H), 3.54- 3.46 (m, 1H), 3.06- 2.99 (m, 3H), 2.86-2.55 (m, 6H), 2.47-2.41 (m, 1H), 2.25- 1.98 (m, 5H), 1.93- 1.36 (m, 9H), 1.27-1.21 (m, 1H), 1.16-1.09 (m, 3H), 0.93- 0.74 (m, 4H), 0.72- 0.61 (m, 1H); 1040.6 [M + H]⁺ . |
| | C166 | 1H NMR (400 MHz, DMSO-d6) δ (ppm) 9.74-9.44 (m, 1H), 8.96- 8.74 (m, 1H), 8.32- 8.22 (m, 1H), 8.11-8.03 (m, 2H), 7.66-7.58 (m, 1H), 7.39-7.27 (m, 6H), 7.25- 7.13 (m, 4H), 6.29- 6.05 (m, 2H), 4.89-4.78 (m, 1H), 4.73-4.46 (m, 3H), 4.18- 3.99 (m, 2H), 3.96- 3.89 (m, 1H), 3.86-3.64 (m, 4H), 3.54-3.49 (m, 1H), 3.06- 3.00 (m, 3H), 2.85- 2.55 (m, 6H), 2.47-2.40 (m, 1H), 2.22-1.22 (m, 15H), 1.14 (d, J = 7.1 Hz, 3H), 0.88- 0.62 (m, 5H); 1040.5 [M + H]⁺ . |
| | C167 | 1H NMR (400 MHz, DMSO-d6) δ (ppm) 9.86-9.31 (m, 1H), 8.92- 8.71 (m, 1H), 8.28 (s, 1H), 8.08 (d, J = 4.1 Hz, 2H), 7.62 (d, J = 8.6 Hz, 1H), 7.42-7.15 (m, 10H), 6.30-5.99 (m, 2H), 4.90- 4.74 (m, 1H), 4.67- 4.44 (m, 2H), 4.14 (t, J = 8.3 Hz, 1H), 3.99-3.89 (m, 1H), 3.88- 3.71 (m, H), 3.47- 3.30 (m, 3H), 3.28- 3.11 (m, 1H), 2.92 (s, 1H), 2.77 (s, 2H), 2.35-1.56 (m, 16H), 1.54- 1.46 (m, 1H), 1.46- 1.38 (m, 1H), 1.14 (d, J = 7.1 Hz, 3H), 0.88-0.72 (m, 3H), 0.60- 0.43 (m, 2H); 998.4 [M + H]⁺ . |
| | C168 | 1H NMR (400 MHz, DMSO-d6) δ (ppm) 9.79-9.43 (m, 1H), 8.90- 8.71 (m, 1H), 8.34- 8.21 (m, 1H), 8.15-8.00 (m, 2H), 7.72-7.53 (m, 1H), 7.42- 7.27 (m, 6H), 7.26- 7.09 (m, 4H), 6.29-6.04 (m, 2H), 4.88-4.38 (m, 5H), 4.20- 4.07 (m, 1H), 3.90- 3.78 (m, 3H), 3.78-3.70 (m, 3H), 3.57-3.55 (m, 1H), 3.42- 3.31 (m, 1H), 2.90- 2.70 (m, 3H), 2.28-1.30 (m, 16H), 1.18-1.08 (m, 3H), 0.83- 0.71 (m, 3H), 0.64- 0.41 (m, 2H); 984.7 [M + H]⁺ . |
| | C169 | 1H NMR (400 MHz, DMSO-d6) δ (ppm) 9.77-9.48 (m, 1H), 8.92- 8.73 (m, 1H), 8.38- 8.20 (m, 1H), 8.17-7.97 (m, 2H), 7.68-7.53 (m, 1H), 7.44 7.26 (m, 6H), 7.26- 7.10 (m, 4H), 6.30-6.09 (m, 2H), 4.88-4.43 (m, 5H), 4.16- 4.11 (m, 1H), 3.97- 3.89 (m, 3H), 3.77-3.71 (m, 2H), 3.57-3.36 (m, 3H), 2.92- 2.69 (m, 3H), 2.28- 2.19 (m, 1H), 2.10-2.01 (m, 4H), 1.95-1.89 (m, 3H), 1.85- 1.75 (m, 3H), 1.72- 1.65 (m, 2H), 1.61-1.47 (m, 3H), 1.18-1.05 (m, 3H), 0.86- 0.77 (m, 3H), 0.64- 0.38 (m, 2H); 984.7 [M + H]⁺ . |
| | C170 | 1H NMR (400 MHz, DMSO-d6) δ (ppm) 8.88-8.72 (m, 1H), 8.29- 8.21 (m, 1H), 8.14- 7.91 (m, 2H), 7.61-7.42 (m, 1H), 7.38-7.26 (m, 4H), 7.25- 7.15 (m, 1H), 6.34- 6.13 (m, 1H), 6.00-5.57 (m, 1H), 4.92-4.66 (m, 2H), 4.54- 4.42 (m, 1H), 4.38- 4.22 (m, 1H), 4.18-4.07 (m, 1H), 4.05-3.51 (m, 6H), 3.24- 2.91 (m, 2H), 2.67- 2.56 (m, 1H), 2.46-1.50 (m, 27H), 1.25-1.16 (m, 3H), 0.90- 0.72 (m, 3H), 0.56- 0.37 (m, 2H); 935.3 [M + H]⁺ . |
| | C172 | 1H NMR (400 MHz, DMSO-d6) δ (ppm) 8.77 (d, J = 7.7 Hz, 1H), 8.27 (s, 1H), 8.10-7.99 (m, 2H), 7.61 (d, J = 8.6 Hz, 1H), 7.40- 7.27 (m, 6H), 7.23-7.12 (m, 4H), 6.27-6.04 (m, 2H), 4.81- 4.65 (m, 2H), 4.52- 4.43 (m, 1H), 4.40 (d, J = 5.4 Hz, 1H), 4.36 (d, J = 5.4 Hz, 1H), 4.32- 4.24 (m, 1H), 4.19 (d, J = 5.4 Hz, 1H), 4.14-4.05 (m, 2H), 3.84-3.67 (m, 2H), 3.59- 3.48 (m, 1H), 2.88- 2.78 (m, 1H), 2.58-2.52 (m, 1H), 2.39-2.31 (m, 1H), 2.22- 1.50 (m, 23H), 1.28 (s, 3H), 1.11 (d, J = 7.0 Hz, 3H), 0.54- 0.38 (m, 2H); 968.7 [M + H]⁺ . |
| | C173 | 1H NMR (400 MHz, DMSO-d6) δ (ppm) 8.86-8.71 (m, 1H), 8.31- 8.21 (m, 1H), 8.08- 7.95 (m, 2H), 7.58-7.47 (m, 1H), 7.41-7.17 (m, 5H), 6.22- 5.93 (m, 2H), 4.84- 4.69 (m, 1H), 4.65-4.24 (m, 6H), 4.21-4.16 (m, 1H), 4.14- 4.06 (m, 2H), 3.94- 3.68 (m, 4H), 3.59-3.47 (m, 1H), 2.90-2.78 (m, 1H), 2.60- 2.51 (m, 1H), 2.41- 1.40 (m, 20H), 1.34-1.17 (m, 6H), 0.85-0.78 (m, 3H), 0.54- 0.39 (m, 2H); 962.4 [M + H]⁺ . |
| | C174 | 1H NMR (400 MHz, DMSO-d6) δ (ppm) 8.78 (d, J = 7.1 Hz, 1H), 8.27 (s, 1H), 8.10-8.00 (m, 2H), 7.60 (d, J = 8.3 Hz, 1H), 7.40- 7.26 (m, 6H), 7.23-7.12 (m, 4H), 6.29-6.07 (m, 2H), 4.76- 4.66 (m, 1H), 4.48 (t, J = 8.4 Hz, 1H), 4.34-4.22 (m, 1H), 4.12 (t, J = 8.6 Hz, 1H), 3.98- 3.84 (m, 1H), 3.81- 3.49 (m, 7H), 3.07-2.92 (m, 2H), 2.83-2.69 (m, 1H), 2.40- 2.19 (m, 4H), 2.14- 2.03 (m, 3H), 2.02-1.90 (m, 6H), 1.88-1.33 (m, 12H), 1.17- 1.05 (m, 3H), 0.52- 0.37 (m, 2H); 1036.6 [M + H]⁺ . |
| | C175 | 1H NMR (400 MHz, DMSO-d6) δ (ppm) 8.80 (d, J = 7.6 Hz, 1H), 8.27 (s, 1H), 8.06 (d, J = 8.4 Hz, 1H), 8.00 (s, 1H), 7.52 (d, J = 8.5 Hz, 1H), 7.37- 7.27 (m, 4H), 7.24- 7.18 (m, 1H), 6.26-6.03 (m, 2H), 4.76-4.55 (m, 3H), 4.48 (t, J = 8.5 Hz, 1H), 4.33- 4.24 (m, 1H), 4.15-4.07 (m, 1H), 4.06-3.94 (m, 2H), 3.78- 3.61 (m, 2H), 3.58- 3.49 (m, 1H), 3.08-2.92 (m, 2H), 2.84-2.71 (m, 1H), 2.37- 2.19 (m, 3H), 2.11- 1.51 (m, 20H), 1.23 (d, J = 7.2 Hz, 3H), 0.86 (t, J = 7.4 Hz, 3H), 0.52 0.39 (m, 2H); 1000.4 [M + H]⁺ . |
| | C176 | 1H NMR (400 MHz, DMSO-d6) δ (ppm) 8.78 (d, J = 7.6 Hz, 1H), 8.26 (s, 1H), 8.03 (d, J = 8.4 Hz, 1H), 7.98 (s, 1H), 7.51 (d, J = 8.5 Hz, 1H), 7.37- 7.27 (m, 4H), 7.24- 7.18 (m, 1H), 6.05-5.89 (m, 1H), 5.68-5.59 (m, 1H), 4.93- 4.82 (m, 1H), 4.76- 4.65 (m, 1H), 4.53-4.43 (m, 1H), 4.35-4.23 (m, 1H), 4.16- 3.99 (m, 3H), 3.79- 3.68 (m, 1H), 3.60-3.48 (m, 2H), 3.09-2.91 (m, 2H), 2.84- 2.69 (m, 1H), 2.39- 2.18 (m, 3H), 2.14-2.04 (m, 3H), 2.02-1.90 (m, 6H), 1.89- 1.64 (m, 5H), 1.64- 1.44 (m, 4H), 1.23 (t, J = 7.1 Hz, 3H), 1.20-1.14 (m, 9H), 0.53- 0.38 (m, 2H); 946.4 [M + H]⁺ . |
| | C177 | 1H NMR (400 MHz, DMSO-d6) δ (ppm) 8.76 (d, J = 7.6 Hz, 1H), 8.25 (s, 1H), 8.06-7.94 (m, 2H), 7.53 (d, J = 8.5 Hz, 1H), 7.39- 7.17 (m, 5H), 5.94 (dd, J = 44.0, 6.6 Hz, 1H), 5.60- 5.49 (m, 1H), 4.87- 4.72 (m, 2H), 4.47 (t, J = 8.5 Hz, 1H), 4.36-4.25 (m, 1H), 4.11 (t, J = 8.6 Hz, 1H), 4.03- 3.92 (m, 2H), 3.78-3.63 (m, 2H), 3.59-3.47 (m, 1H), 2.94 (s, 1H), 2.44-1.52 (m, 23H), 1.25- 1.13 (m, 9H), 1.09 (d, J = 6.3 Hz, 3H), 0.55-0.39 (m, 2H); 934.9 [M + H]⁺ . |
| | C178 | 1H NMR (400 MHz, DMSO-d6) δ (ppm) 8.79 (d, J = 7.7 Hz, 1H), 8.25 (s, 1H), 8.09-7.92 (m, 2H), 7.53 (d, J = 8.5 Hz, 1H), 7.37- 7.27 (m, 4H), 7.24-7.17 (m, 1H), 6.05-5.82 (m, 1H), 5.65- 5.51 (m, 1H), 4.89- 4.62 (m, 2H), 4.53-4.41 (m, 1H), 4.35-4.21 (m, 1H), 4.18- 4.08 (m, 1H), 4.05- 3.66 (m, 6H), 3.59-3.49 (m, 1H), 3.11-2.97 (m, 1H), 2.76- 2.65 (m, 1H), 2.59- 2.52 (m, 1H), 2.44-1.43 (m, 21H), 1.27-1.16 (m, 6H), 1.03- 0.93 (m, 3H), 0.86- 0.78 (m, 3H), 0.56-0.39 (m, 2H); 910.6 [M + H]⁺ . |
| | C179 | 1H NMR (400 MHz, DMSO-d6) δ (ppm) 9.67-9.42 (m, 1H), 8.92- 8.66 (m, 1H), 8.27 (s, 1H), 8.15-7.98 (m, 2H), 7.61 (d, J = 8.2 Hz, 1H), 7.41- 7.28 (m, 6H), 7.25-7.11 (m, 4H), 6.47-5.94 (m, 3H), 4.87- 4.69 (m, 1H), 4.65- 4.43 (m, 2H), 4.33-3.51 (m, 13H), 3.29-2.99 (m, 6H), 2.81- 2.69 (m, 3H), 2.39- 1.74 (m, 15H), 1.61-1.51 (m, 1H), 1.23-1.12 (m, 3H), 0.60- 0.39 (m, 2H); 1021.5 [M + H]⁺ . |
| | C180 | 1H NMR (400 MHz, DMSO-d6) δ (ppm) 9.78-9.39 (m, 1H), 8.92- 8.72 (m, 1H), 8.35- 8.00 (m, 3H), 7.61 (d, J = 8.6 Hz, 1H), 7.38-7.27 (m, 6H), 7.25- 7.12 (m, 4H), 6.38- 6.00 (m, 3H), 4.81 (s, 1H), 4.60- 4.50 (m, 2H), 4.18- 4.08 (m, 1H), 3.87-3.66 (m, 4H), 3.60-3.51 (m, 2H), 3.31 3.09 (m, 2H), 2.85- 2.66 (m, 1H), 2.48-1.49 (m, 16H), 1.21-1.13 (m, 3H), 0.88- 0.74 (m, 9H), 0.56- 0.44 (m, 2H); 979.0 [M + H]⁺ . |
| | C181 | 1H NMR (400 MHz, DMSO-d6) δ (ppm) 9.84-9.20 (m, 1H), 8.89- 8.71 (m, 1H), 8.30- 8.25 (m, 1H), 8.13-8.09 (m, 2H), 7.62 (d, J = 8.6 Hz, 1H), 7.42- 7.29 (m, 6H), 7.25- 7.17 (m, 4H), 6.50-6.36 (m, 1H), 6.34-6.01 (m, 1H), 4.87- 4.78 (m, 1H), 4.60- 4.50 (m, 2H), 4.17-4.11 (m, 1H), 4.02-3.88 (m, 4H), 3.78- 3.73 (m, 1H), 3.60- 3.46 (m, 2H), 3.39-3.10 (m, 3H), 2.83-2.68 (m, 3H), 2.45- 1.74 (m, 19H), 1.63- 1.42 (m, 3H), 0.81 (t, J = 7.4 Hz, 3H), 0.49 (s, 2H); 977.8 [M + H]⁺ . |
| | C182 | 1H NMR (400 MHz, DMSO-d6) δ (ppm) 8.87-8.71 (m, 1H), 8.27 (s, 1H), 8.14-7.96 (m, 2H), 7.60 (d, J = 8.6 Hz, 1H), 7.40- 7.25 (m, 6H), 7.24-7.12 (m, 4H), 6.33-6.09 (m, 2H), 4.90- 4.66 (m, 2H), 4.47 (t, J = 8.5 Hz, 1H), 4.35-4.22 (m, 1H), 4.12 (t, J = 8.6 Hz, 1H), 4.02- 3.67 (m, 4H), 3.61- 3.45 (m, 1H), 3.11-2.95 (m, 4H), 2.64-2.55 (m, 1H), 2.40- 1.49 (m, 21H), 1.19- 1.13 (m, 3H), 1.09-1.01 (m, 6H), 0.55-0.39 (m, 2H); 989.0 [M + H]⁺ . |
| | C183 | 1H NMR (400 MHz, DMSO-d6) δ (ppm) 9.82-9.44 (m, 1H), 8.96- 8.74 (m, 1H), 8.27 (m, 1H), 8.12- 8.00 (m, 2H), 7.67- 7.57 (m, 1H), 7.43-7.28 (m, 6H), 7.26-7.11 (m, 4H), 6.31- 6.03 (m, 2H), 5.04- 4.75 (m, 3H), 4.65-4.48 (m, 3H), 4.21-4.08 (m, 1H), 3.81- 3.69 (m, 2H), 3.60- 3.50 (m, 1H), 2.81-2.72 (m, 1H), 2.68-2.52 (m, 8H), 2.47- 1.49 (m, 20H), 1.11 (d, J = 7.1 Hz, 3H), 0.67-0.41 (m, 2H); 1032.4 [M + H]⁺ . |
| | C184 | 1H NMR (400 MHz, DMSO-d6) δ (ppm) 8.86-8.75 (m, 1H), 8.27 (s, 1H), 8.12-7.98 (m, 2H), 7.60 (d, J = 7.5 Hz, 1H), 7.40- 7.10 (m, 10H), 6.32-6.05 (m, 2H), 4.92-4.64 (m, 2H), 4.55- 4.41 (m, 1H), 4.37- 4.21 (m, 1H), 4.17-4.05 (m, 1H), 4.03-3.46 (m, 8H), 3.03- 2.95 (m, 1H), 2.64- 2.55 (m, 1H), 2.45-1.31 (m, 25H), 1.19-1.07 (m, 3H), 0.57- 0.39 (m, 2H); 986.6 [M + H]⁺ . |
| | C185 | 1H NMR (400 MHz, DMSO-d6) δ (ppm) 8.84-8.74 (m, 1H), 8.27 (s, 1H), 8.11-8.01 (m, 2H), 7.65- 7.55 (m, 1H), 7.42- 7.26 (m, 6H), 7.25-7.10 (m, 4H), 6.30-6.06 (m, 2H), 4.90- 4.66 (m, 2H), 4.47 (t, J = 8.6 Hz, 1H), 4.35-4.22 (m, 1H), 4.12 (t, J = 8.6 Hz, 1H), 4.01- 3.51 (m, 8H), 3.39- 3.33 (m, 1H), 3.05-2.95 (m, 1H), 2.65-2.55 (m, 1H), 2.41- 1.39 (m, 24H), 1.13 (d, J = 7.1 Hz, 3H), 0.58-0.37 (m, 2H); 987.1 [M + H]⁺ . |
| | C186 | 1H NMR (400 MHz, DMSO-d6) δ (ppm) 9.87-9.47 (m, 1H), 8.95- 8.77 (m, 1H), 8.37- 8.23 (m, 1H), 8.13-8.03 (m, 2H), 7.61 (d, J = 8.6 Hz, 1H), 7.45- 7.27 (m, 6H), 7.25- 7.12 (m, 4H), 6.27-6.08 (m, 2H), 5.02-4.79 (m, 2H), 4.62- 4.50 (m, 2H), 4.22- 4.09 (m, 2H), 3.87-3.79 (m, 3H), 3.71-3.50 (m, 5H), 3.43- 3.01 (m, 3H), 2.82- 2.64 (m, 2H), 2.64-2.55 (m, 3H), 2.48-2.40 (m, 2H), 2.31- 1.50 (m, 14H), 1.48- 1.37 (m, 2H), 1.19-1.04 (m, 5H), 0.65-0.44 (m, 2H); 500.9 [M/2 + H]⁺ . |
| | C187 | 1H NMR (400 MHz, DMSO-d6) δ (ppm) 9.77-9.41 (m, 1H), 9.01- 8.67 (m, 1H), 8.32- 8.18 (m, 1H), 8.13-7.96 (m, 2H), 7.67-7.54 (m, 1H), 7.39- 7.28 (m, 6H), 7.24- 7.11 (m, 4H), 6.28-5.92 (m, 2H), 5.09-4.71 (m, 2H), 4.66- 4.45 (m, 2H), 4.22- 4.07 (m, 1H), 3.87-3.66 (m, 3H), 3.62-3.52 (m, 3H), 2.82- 2.63 (m, 2H), 2.63- 2.55 (m, 3H), 2.48-2.41 (m, 2H), 2.34-1.71 (m, 13H), 1.70- 1.36 (m, 5H), 0.87- 0.82 (m, 2H), 0.80-0.69 (m, 5H), 0.67-0.60 (m, 2H), 0.59- 0.44 (m, 2H); 972.6 [M + H]⁺ . |
| | C188 | 1H NMR (400 MHz, DMSO-d6) δ (ppm) 8.91-8.74 (m, 1H), 8.29 (s, 1H), 8.18-8.03 (m, 2H), 7.62 (d, J = 6.7 Hz, 1H), 7.46- 7.27 (m, 6H), 7.27-7.13 (m, 4H), 6.30-6.19 (m, 1H), 6.19- 6.05 (m, 1H), 4.95- 4.67 (m, 2H), 4.49 (t, J = 8.5 Hz, 1H), 4.40-4.23 (m, 1H), 4.14 (t, J = 8.5 Hz, 1H), 4.07- 3.67 (m, 4H), 3.64-3.51 (m, 1H), 3.10-2.95 (m, 1H), 2.68- 2.57 (m, 1H), 2.45- 2.32 (m, 2H), 2.25 (t, J = 11.5 Hz, 1H), 2.20- 2.06 (m, 2H), 2.05- 1.95 (m, 6H), 1.91-1.51 (m, 10H), 1.43-1.30 (m, 2H), 1.19- 1.12 (m, 3H), 0.94- 0.74 (m, 7H), 0.60-0.37 (m, 2H); 972.6 [M + H]⁺ . |
| | C189 | 1H NMR (400 MHz, DMSO-d6) δ (ppm) 9.71-9.44 (m, 1H), 8.96- 8.76 (m, 1H), 8.32- 8.24 (m, 1H), 8.10-8.03 (m, 2H), 7.61 (d, J = 8.2 Hz, 1H), 7.41- 7.29 (m, 6H), 7.25- 7.09 (m, 4H), 6.30-5.96 (m, 2H), 5.03-4.78 (m, 2H), 4.61- 4.49 (m, 2H), 4.22- 4.13 (m, 2H), 3.96-3.91 (m, 1H), 3.78-3.72 (m, 2H), 3.65- 3.54 (m, 2H), 3.12- 2.71 (m, 1H), 2.63-2.55 (m, 3H), 2.50-1.81 (m, 14H), 1.81- 0.95 (m, 10H), 0.88- 0.65 (m, 6H), 0.63-0.46 (m, 2H); 972.6 [M + H]⁺ . |
| | C190 | 1H NMR (400 MHz, DMSO-d6) δ (ppm) 8.78 (d, J = 7.6 Hz, 1H), 8.25 (s, 1H), 8.07-7.90 (m, 2H), 7.52 (d, J = 8.7 Hz, 1H), 7.37- 7.26 (m, 4H), 7.23-7.18 (m, 1H), 5.97-5.73 (m, 1H), 5.52- 5.38 (m, 1H), 4.89- 4.66 (m, 2H), 4.53-4.38 (m, 2H), 4.33-4.23 (m, 1H), 4.16- 4.06 (m, 1H), 4.03- 3.92 (m, 2H), 3.78-3.68 (m, 1H), 3.65-3.50 (m, 2H), 3.05- 2.94 (m, 1H), 2.62- 2.57 (m, 1H), 2.44-1.35 (m, 33H), 1.08-0.97 (m, 3H), 0.90- 0.82 (m, 3H), 0.55- 0.38 (m, 2H); 950.8 [M + H]⁺ . |
| | C191 | 1H NMR (400 MHz, DMSO-d6) δ (ppm) 8.80 (d, J = 7.7 Hz, 1H), 8.25 (d, J = 2.8 Hz, 1H), 8.06-7.99 (m, 1H), 7.95 (s, 1H), 7.49 (d, J = 8.6 Hz, 1H), 7.37-7.26 (m, 4H), 7.24- 7.14 (m, 1H), 6.02- 5.80 (m, 1H), 5.64-5.48 (m, 1H), 4.97-4.66 (m, 3H), 4.51- 4.43 (m, 1H), 4.34- 4.24 (m, 1H), 4.14-4.08 (m, 1H), 4.02-3.47 (m, 5H), 3.04- 2.95 (m, 1H), 2.65- 2.55 (m, 1H), 2.44-1.45 (m, 31H), 1.30-1.18 (m, 3H), 0.92- 0.81 (m, 3H), 0.56- 0.38 (m, 2H); 936.9 [M + H]⁺ . |
| | C192 | 1H NMR (400 MHz, DMSO-d6) δ (ppm) 8.79 (d, J = 7.7 Hz, 1H), 8.25 (s, 1H), 8.08-7.89 (m, 2H), 7.52 (d, J = 8.6 Hz, 1H), 7.37- 7.26 (m, 4H), 7.24-7.17 (m, 1H), 6.02-5.76 (m, 1H), 5.61- 5.43 (m, 1H), 4.90- 4.66 (m, 2H), 4.63-4.53 (m, 1H), 4.51-4.42 (m, 1H), 4.35- 4.22 (m, 1H), 4.18- 4.05 (m, 1H), 3.95-3.48 (m, 5H), 3.06-2.93 (m, 1H), 2.65- 2.56 (m, 1H), 2.44- 1.41 (m, 23H), 1.29-1.13 (m, 9H), 0.89-0.75 (m, 3H), 0.56- 0.37 (m, 2H); 910.7 [M + H]⁺ . |
| | C193 | 1H NMR (400 MHz, DMSO-d6) δ (ppm) 9.76-9.42 (m, 1H), 8.98- 8.75 (m, 1H), 8.31- 8.21 (m, 1H), 8.09-7.96 (m, 2H), 7.53 (d, J = 8.3 Hz, 1H), 7.39- 7.27 (m, 4H), 7.26- 7.19 (m, 1H), 6.22-5.95 (m, 2H), 4.97-4.78 (m, 2H), 4.60- 4.48 (m, 4H), 4.19- 4.11 (m, 1H), 3.89-3.69 (m, 4H), 3.63-3.49 (m, 3H), 3.45- 2.99 (m, 1H), 2.81- 2.72 (m, 1H), 2.66-2.55 (m, 4H), 2.46-2.41 (m, 1H), 2.29- 1.51 (m, 14H), 1.30- 1.24 (m, 3H), 0.88-0.80 (m, 9H), 0.63-0.44 (m, 2H); 978.9 [M + H]⁺ . |
| | C194 | 1H NMR (400 MHz, DMSO-d6) δ (ppm) 8.80 (d, J = 7.6 Hz, 1H), 8.35- 8.19 (m, 1H), 8.08- 7.91 (m, 2H), 7.59-7.47 (m, 1H), 7.39-7.16 (m, 5H), 6.13- 5.86 (m, 1H), 5.77- 5.50 (m, 1H), 4.93-4.65 (m, 2H), 4.47 (t, J = 8.5 Hz, 1H), 4.35- 4.23 (m, 1H), 4.16- 3.46 (m, 10H), 3.29-3.21 (m, 3H), 3.06-2.93 (m, 1H), 2.63- 2.56 (m, 1H), 2.43- 1.44 (m, 23H), 1.28-1.18 (m, 3H), 0.90-0.76 (m, 3H), 0.58- 0.38 (m, 2H); 926.8 [M + H]⁺ . |
| | C195 | 1H NMR (400 MHz, DMSO-d6) δ (ppm) 10.07-9.41 (m, 1H), 8.98- 8.73 (m, 1H), 8.31- 8.23 (m, 1H), 8.11-8.03 (m, 2H), 7.61 (d, J = 8.3 Hz, 1H), 7.40- 7.28 (m, 6H), 7.25- 7.10 (m, 4H), 6.31-6.04 (m, 2H), 5.05-4.90 (m, 1H), 4.88- 4.76 (m, 2H), 4.75- 4.65 (m, 1H), 4.64-4.47 (m, 3H), 4.23-4.06 (m, 2H), 3.75- 3.70 (m, 1H), 3.56- 3.50 (m, 1H), 3.45-3.02 (m, 1H), 2.81-2.62 (m, 2H), 2.62- 2.55 (m, 3H), 2.48- 2.41 (m, 1H), 2.39-2.17 (m, 4H), 2.16-1.49 (m, 20H), 1.17- 0.88 (m, 3H), 0.64- 0.45 (m, 2H); 996.5 [M + H]⁺ . |
| | C196 | 1H NMR (400 MHz, DMSO-d6) δ (ppm) 9.93-9.45 (m, 1H), 8.95- 8.76 (m, 1H), 8.31- 8.24 (m, 1H), 8.12-8.02 (m, 2H), 7.61 (d, J = 8.5 Hz, 1H), 7.40- 7.06 (m, 10H), 6.25- 5.87 (m, 2H), 4.99-4.78 (m, 2H), 4.58-4.46 (m, 2H), 4.16- 4.10 (m, 2H), 3.94- 3.89 (m, 1H), 3.79-3.74 (m, 1H), 3.70-3.62 (m, 1H), 3.57- 3.51 (m, 1H), 3.44- 3.33 (m, 1H), 3.09-2.73 (m, 1H), 2.70-2.56 (m, 4H), 2.48- 1.43 (m, 19H), 1.33- 1.21 (m, 1H), 0.88-0.78 (m, 5H), 0.73-0.68 (m, 1H), 0.65- 0.47 (m, 5H); 972.6 [M + H]⁺ . |
| | C197 | 1H NMR (400 MHz, DMSO-d6) δ (ppm) 8.85-8.72 (m, 1H), 8.31- 8.24 (m, 1H), 8.10- 8.01 (m, 2H), 7.60 (d, J = 8.6 Hz, 1H), 7.40-7.27 (m, 6H), 7.25- 7.10 (m, 4H), 6.30- 6.19 (m, 1H), 6.18-6.09 (m, 1H), 5.20-5.03 (m, 1H), 4.90- 4.65 (m, 2H), 4.52- 4.44 (m, 1H), 4.34-4.24 (m, 1H), 4.16-4.07 (m, 1H), 3.88- 3.63 (m, 5H), 3.58- 3.42 (m, 2H), 3.06-2.96 (m, 1H), 2.66-2.51 (m, 2H), 2.43- 2.29 (m, 2H), 2.27- 2.20 (m, 1H), 2.16-1.51 (m, 19H), 1.19-1.07 (m, 3H), 0.58- 0.39 (m, 2H); 972.5 [M + H]⁺ . |
| | C198 | 1H NMR (400 MHz, DMSO-d6) δ (ppm) 8.83-8.75 (m, 1H), 8.27 (s, 1H), 8.11-7.96 (m, 2H), 7.60 (d, J = 8.2 Hz, 1H), 7.40- 7.10 (m, 10H), 6.34-6.04 (m, 2H), 5.19-5.01 (m, 1H), 4.90- 4.66 (m, 2H), 4.54- 4.44 (m, 1H), 4.35-4.24 (m, 1H), 4.12 (t, J = 8.5 Hz, 1H), 3.82- 3.48 (m, 7H), 3.05- 2.93 (m, 1H), 2.63-2.56 (m, 1H), 2.42-2.30 (m, 2H), 2.27- 2.20 (m, 1H), 2.16- 1.48 (m, 20H), 1.17-1.04 (m, 3H), 0.55-0.40 (m, 2H); 972.8 [M + H]⁺ . |
| | C199 | 1H NMR (400 MHz, DMSO-d6) δ (ppm) 8.85-8.73 (m, 1H), 8.27 (d, J = 4.6 Hz, 1H), 8.11- 8.02 (m, 2H), 7.60 (t, J = 7.7 Hz, 1H), 7.39-7.27 (m, 6H), 7.23- 7.11 (m, 4H), 6.35- 6.07 (m, 2H), 4.90-4.66 (m, 2H), 4.47 (t, J = 8.6 Hz, 1H), 4.34- 4.24 (m, 1H), 4.16- 3.69 (m, 5H), 3.60-3.47 (m, 1H), 3.05-2.95 (m, 1H), 2.64- 2.55 (m, 1H), 2.41- 2.31 (m, 2H), 2.23 (t, J = 11.6 Hz, 1H), 2.16- 2.10 (m, 1H), 2.03- 1.91 (m, 7H), 1.89-1.61 (m, 6H), 1.60-1.51 (m, 3H), 1.35- 1.14 (m, 9H), 0.99 (d, J = 7.1 Hz, 1H), 0.54-0.40 (m, 2H); 976.3 [M + H]⁺ . |
| | C200 | 1H NMR (400 MHz, DMSO-d6) δ (ppm) 8.84-8.73 (m, 1H), 8.31- 8.23 (m, 1H), 8.11- 8.03 (m, 2H), 7.63-7.56 (m, 1H), 7.38-7.27 (m, 6H), 7.23- 7.14 (m, 4H), 6.24- 6.06 (m, 2H), 4.90-4.66 (m, 2H), 4.52-4.40 (m, 1H), 4.36- 4.22 (m, 1H), 4.18- 4.06 (m, 1H), 3.92-3.84 (m, 2H), 3.78-3.51 (m, 6H), 3.06- 2.94 (m, 1H), 2.65- 2.56 (m, 1H), 2.44-1.42 (m, 25H), 1.22-1.11 (m, 1H), 0.97- 0.93 (m, 2H), 0.55- 0.39 (m, 2H); 987.8 [M + H]⁺ . |
| | C201 | 1H NMR (400 MHz, DMSO-d6) δ (ppm) 8.79 (t, J = 6.8 Hz, 1H), 8.31- 8.23 (m, 1H), 8.12- 8.00 (m, 2H), 7.65-7.54 (m, 1H), 7.43-7.26 (m, 6H), 7.24- 7.07 (m, 4H), 6.31- 6.07 (m, 2H), 4.92-4.63 (m, 2H), 4.47 (t, J = 8.6 Hz, 1H), 4.35- 4.24 (m, 1H), 4.12 (t, J = 8.5 Hz, 1H), 3.99-3.48 (m, 8H), 3.37-3.33 (m, 1H), 3.04- 2.95 (m, 1H), 2.63- 2.54 (m, 1H), 2.42-1.43 (m, 24H), 1.22-0.91 (m, 3H), 0.57- 0.37 (m, 2H); 987.1 [M + H]⁺ . |
| | C202 | 1H NMR (400 MHz, DMSO-d6) δ (ppm) 8.86-8.71 (m, 1H), 8.30- 8.25 (m, 1H), 8.14- 7.99 (m, 2H), 7.68-7.50 (m, 1H), 7.42-7.08 (m, 10H), 6.33- 6.02 (m, 2H), 4.94- 4.59 (m, 2H), 4.54-4.39 (m, 1H), 4.35-4.21 (m, 1H), 4.18- 4.03 (m, 1H), 3.94- 3.64 (m, 6H), 3.60-3.47 (m, 1H), 3.25-3.14 (m, 2H), 3.06-2.92 (m, 1H), 2.64- 2.56 (m, 1H), 2.45- 1.32 (m, 24H), 1.22-0.91 (m, 5H), 0.60-0.35 (m, 2H); 1000.6 [M + H]⁺ . |
| | C203 | 1H NMR (400 MHz, DMSO-d6) δ (ppm) 9.70-9.41 (m, 1H), 8.98- 8.71 (m, 1H), 8.35- 8.23 (m, 1H), 8.17-8.03 (m, 2H), 7.62 (d, 1H), 7.48-7.11 (m, 10H), 6.63-6.43 (m, 1H), 4.88- 4.75 (m, 1H), 4.74- 4.44 (m, 3H), 4.43-4.30 (m, 1H), 4.25-3.87 (m, 4H), 3.84 3.46 (m, 5H), 3.18- 3.05 (m, 1H), 2.83-2.62 (m, 4H), 2.34-1.69 (m, 13H), 1.56- 1.41 (m, 3H), 1.33- 1.13 (m, 4H), 0.85-0.73 (m, 3H), 0.65-0.38 (m, 2H); 1072.7 [M + H]⁺ . |
| | C204 | 1H NMR (400 MHz, DMSO-d6) δ (ppm) 8.91-8.65 (m, 1H), 8.26 (s, 1H), 8.14-7.90 (m, 2H), 7.54 (d, J = 8.6 Hz, 1H), 7.38- 7.15 (m, 5H), 6.42-6.17 (m, 1H), 4.77-4.40 (m, 5H), 4.39- 4.20 (m, 2H), 4.19- 4.04 (m, 1H), 3.97-3.82 (m, 2H), 3.80-3.65 (m, 1H), 3.61- 3.44 (m, 1H), 3.06- 2.93 (m, 1H), 2.72-2.52 (m, 5H), 2.44-2.10 (m, 5H), 2.01- 1.44 (m, 17H), 1.31- 1.23 (m, 3H), 0.85-0.76 (m, 3H), 0.58-0.31 (m, 2H); 1030.4 [M + H]⁺ . |
| | C205 | 1H NMR (400 MHz, DMSO-d6) δ (ppm) 10.09-9.53 (m, 1H), 8.98- 8.72 (m, 1H), 8.32- 8.20 (m, 1H), 8.12-7.95 (m, 2H), 7.60-7.49 (m, 1H), 7.42- 7.29 (m, 4H), 7.26- 7.17 (m, 1H), 6.08-5.86 (m, 1H), 5.73-5.56 (m, 1H), 4.90- 4.45 (m, 5H), 4.26- 3.85 (m, 6H), 3.80-3.71 (m, 3H), 2.81-2.72 (m, 1H), 2.63- 2.56 (m, 3H), 2.37- 1.70 (m, 14H), 1.69-1.33 (m, 4H), 1.30-1.17 (m, 10H), 0.88- 0.76 (m, 6H), 0.62- 0.43 (m, 2H); 1004.6 [M + H]⁺ . |
| | C206 | 1H NMR (400 MHz, DMSO-d6) δ (ppm) 9.91-9.42 (m, 1H), 8.92- 8.71 (m, 1H), 8.31- 8.19 (m, 1H), 8.13-7.96 (m, 2H), 7.64-7.48 (m, 1H), 7.39- 7.27 (m, 4H), 7.26- 7.19 (m, 1H), 6.29-6.01 (m, 1H), 4.87-4.76 (m, 1H), 4.72- 4.46 (m, 3H), 4.35- 3.87 (m, 6H), 3.81-3.48 (m, 6H), 2.85-2.75 (m, 1H), 2.73- 2.65 (m, 1H), 2.63- 2.52 (m, 6H), 2.47-2.43 (m, 1H), 2.28-1.49 (m, 15H), 1.34- 1.11 (m, 6H), 0.92- 0.81 (m, 3H), 0.65-0.39 (m, 2H); 976.8 [M + H]⁺ . |
| | C207 | 1H NMR (400 MHz, DMSO-d6) δ (ppm) 9.77-9.36 (m, 1H), 8.95- 8.67 (m, 1H), 8.30- 8.19 (m, 1H), 8.10-7.97 (m, 2H), 7.61-7.49 (m, 1H), 7.45- 7.06 (m, 5H), 6.30- 6.06 (m, 1H), 4.85-4.75 (m, 1H), 4.71-4.41 (m, 3H), 4.39- 4.27 (m, 1H), 4.23- 4.07 (m, 1H), 4.01 (t, J = 6.5 Hz, 2H), 3.96-3.84 (m, 2H), 3.83- 3.71 (m, 1H), 3.65- 3.41 (m, 7H), 3.20-3.14 (m, 1H), 2.99-2.91 (m, 1H), 2.84- 2.74 (m, 1H), 2.68- 2.58 (m, 3H), 2.30-1.46 (m, 19H), 1.16 (t, J = 7.0 Hz, 3H), 0.89 (t, J = 7.4 Hz, 3H), 0.59-0.36 (m, 2H); 988.8 [M+ H]+ |
| | C208 | 1H NMR (400 MHz, DMSO-d6) δ (ppm) 10.09-9.39 (m, 1H), 8.98- 8.75 (m, 1H), 8.32- 8.24 (m, 1H), 8.12-8.02 (m, 2H), 7.66-7.58 (m, 1H), 7.40- 7.28 (m, 6H), 7.25- 7.14 (m, 4H), 6.29-6.05 (m, 2H), 4.95-4.81 (m, 2H), 4.59- 4.52 (m, 2H), 4.20- 4.10 (m, 1H), 3.83-3.47 (m, 5H), 3.45-3.02 (m, 1H), 2.84- 2.53 (m, 6H), 2.48- 2.43 (m, 1H), 2.31-1.40 (m, 26H), 1.18-1.05 (m, 3H), 0.67- 0.39 (m, 2H); 1011.0 [M+ H]+ |
| | C209 | 1H NMR (400 MHz, DMSO-d6) δ (ppm) 8.85-8.72 (m, 1H), 8.27 (s, 1H), 8.11-7.99 (m, 2H), 7.66- 7.57 (m, 1H), 7.41- 7.11 (m, 10H), 6.27-6.05 (m, 2H), 4.95-4.66 (m, 3H), 4.51- 4.42 (m, 1H), 4.35- 4.23 (m, 1H), 4.18-4.05 (m, 1H), 3.86-3.67 (m, 2H), 3.60- 3.48 (m, 1H), 3.06- 2.93 (m, 1H), 2.43-1.48 (m, 26H), 1.19-1.05 (m, 3H), 0.57- 0.27 (m, 6H); |
| | C210 | 982.5 [M + H]⁺ . 1H NMR (400 MHz, DMSO-d6) δ (ppm) 8.77 (d, J = 7.6 Hz, 1H), 8.28 (s, 1H), 8.13-7.99 (m, 2H), 7.61 (d, J = 8.5 Hz, 1H), 7.39-7.26 (m, 6H), 7.24-7.13 (m, 4H), 6.24-6.06 (m, 2H), 4.78-4.64 (m, 1H), 4.48 (t, J = 8.6 Hz, 1H), 4.36-4.23 (m, 1H), 4.11 (t, J = 8.6 Hz, 1H), 3.96-3.85 (m, 2H), 3.77-3.63 (m, 2H), 3.59-3.46 (m, 1H), 3.07-2.90 (m, 2H), 2.84-2.69 (m, 1H), 2.38-2.17 (m, 3H), 2.14-2.03 (m, 3H), 2.02-1.90 (m, 6H), 1.86-1.44 (m, 11H), 0.94 (d, J = 7.1 Hz, 3H), 0.81 (t, J = 7.4 Hz, 3H), 0.54-0.37 (m, 2H); 994 [M + H]⁺ . |
| | C211 | 1H NMR (400 MHz, DMSO) δ 8.82-8.71 (m, 1H), 8.24 (s, 1H), 8.02 (d, J = 8.4 Hz, 1H), 7.95 (s, 1H), 7.50 (d, J = 8.5 Hz, 1H), 7.36-7.26 (m, 4H), 7.24- 7.18 (m, 1H), 5.98- 5.82 (m, 1H), 5.37-5.25 (m, 1H), 4.75-4.64 (m, 1H), 4.57- 4.43 (m, 2H), 4.35- 4.22 (m, 1H), 4.16-4.06 (m, 1H), 4.01-3.94 (m, 2H), 3.78- 3.66 (m, 3H), 3.59- 3.49 (m, 1H), 3.04-2.93 (m, 1H), 2.70-2.59 (m, 2H), 2.44- 2.30 (m, 2H), 2.26- 2.19 (m, 1H), 2.18-2.08 (m, 2H), 2.03-1.91 (m, 6H), 1.90- 1.50 (m, 12H), 1.36- 1.30 (m, 3H), 1.27-1.23 (m, 3H), 0.91-0.85 (m, 3H), 0.84- 0.78 (m, 6H), 0.53- 0.38 (m, 2H); 1004.7 [M + H]⁺ . |
| | C212 | 1H NMR (400 MHz, DMSO) δ 8.87-8.66 (m, 1H), 8.25 (s, 1H), 8.08-7.88 (m, 2H), 7.50 (d, J = 8.5 Hz, 1H), 7.40- 7.17 (m, 5H), 5.99- 5.73 (m, 1H), 5.30-5.16 (m, 1H), 4.76-4.63 (m, 2H), 4.57- 4.42 (m, 2H), 4.34- 4.25 (m, 1H), 4.15-4.07 (m, 1H), 4.02-3.90 (m, 2H), 3.81- 3.66 (m, 1H), 3.57- 3.45 (m, 1H), 3.04-2.93 (m, 1H), 2.72-2.59 (m, 2H), 2.45- 2.29 (m, 2H), 2.24- 2.07 (m, 3H), 2.02-1.92 (m, 6H), 1.89-1.48 (m, 11H), 1.34- 1.10 (m, 12H), 0.92- 0.81 (m, 3H), 0.54-0.36 (m, 2H); 990.9 [M+ H]+ |
| | C213 | 1H NMR (400 MHz, DMSO) δ 8.76 (m, 1H), 8.24 (s, 1H), 8.02 (d, J = 8.4 Hz, 1H), 7.94 (s, 1H), 7.49 (d, J = 8.5 Hz, 1H), 7.39- 7.25 (m, 4H), 7.25- 7.16 (m, 1H), 6.00-5.79 (m, 1H), 5.42-5.25 (m, 1H), 4.77- 4.64 (m, 1H), 4.61- 4.41 (m, 2H), 4.35-4.25 (m, 1H), 4.17-4.08 (m, 1H), 4.02- 3.86 (m, 4H), 3.82- 3.65 (m, 1H), 3.58-3.48 (m, 1H), 3.07-2.94 (m, 1H), 2.73- 2.55 (m, 2H), 2.45- 2.29 (m, 2H), 2.28-2.19 (m, 1H), 2.17-2.09 (m, 2H), 2.05. 1.92 (m, 6H), 1.90- 1.66 (m, 5H), 1.62-1.44 (m, 8H), 1.36-1.20 (m, 8H), 0.93- 0.85 (m, 3H), 0.84- 0.77 (m, 3H), 0.54-0.37 (m, 2H); 1004 [M+ H]+ |
| | C214 | 1H NMR (400 MHz, DMSO) § 8.79-8.72 (m, 1H), 8.24 (s, 1H), 8.02 (d, J = 8.4 Hz, 1H), 7.95 (s, 1H), 7.49 (d, J = 8.4 Hz, 1H), 7.36-7.27 (m, 4H), 7.23- 7.18 (m, 1H), 5.98- 5.81 (m, 1H), 5.37-5.26 (m, 1H), 4.76-4.66 (m, 1H), 4.57- 4.44 (m, 2H), 4.33- 4.23 (m, 1H), 4.16-4.07 (m, 1H), 4.00-3.85 (m, 4H), 3.76- 3.68 (m, 1H), 3.58- 3.49 (m, 1H), 3.06-2.92 (m, 1H), 2.70-2.59 (m, 2H), 2.45- 2.30 (m, 2H), 2.27- 2.19 (m, 1H), 2.18-2.10 (m, 2H), 2.02-1.92 (m, 6H), 1.91- 1.61 (m, 6H), 1.60- 1.50 (m, 7H), 1.37-1.30 (m, 3H), 1.26-1.21 (m, 3H), 0.91- 0.79 (m, 6H), 0.52- 0.40 (m, 2H); 990.9 [M + H]⁺ . |
| | C215 | 1H NMR (400 MHz, DMSO) & 8.79-8.70 (m, 1H), 8.27-8.21 (m, 1H), 8.06- 7.99 (d, J = 8.4 Hz, 1H), 7.95 (s, 1H), 7.53-7.44 (d, J = 8.5 Hz, 1H), 7.38- 7.27 (m, 4H), 7.21 (m, 1H), 5.98- 5.77 (m, 1H), 5.39- 5.30 (m, 1H), 4.75-4.66 (m, 1H), 4.59-4.43 (m, 2H), 4.37- 4.23 (m, 1H), 4.18- 4.08 (m, 1H), 4.07-3.93 (m, 4H), 3.73 (m, 1H), 3.58-3.50 (m, 1H), 3.05-2.95 (m, 1H), 2.70- 2.58 (m, 2H), 2.48- 2.45 (m, 1H), 2.45-2.38 (m, 1H), 2.36-2.30 (m, 1H), 2.27- 2.19 (m, 1H), 2.18- 2.08 (m, 2H), 2.02-1.92 (m, 6H), 1.88-1.63 (m, 5H), 1.60- 1.51 (m, 5H), 1.35- 1.27 (m, 3H), 1.24-1.14 (m, 6H), 0.91-0.85 (m, 3H), 0.53- 0.40 (m, 2H); 976.8 [M + H]⁺ . |

### Example 2: SH2 Scan Assay

SH2 domain-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining SH2 domains were produced in HEK-293 cells and subsequently tagged with DNA for qPCR detection. Streptavidin-coated magnetic beads were treated with biotinylated small molecule ligands for 30 minutes at room temperature to generate affinity resins for SH2 domain assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining SH2 domains, liganded affinity beads, and test compounds in 1× binding buffer (10 mM HEPES, 50 mM NaCl, 1 mM EDTA, 0.01% Tween 20, 6 mM DTT). Test compounds were prepared as 111× stocks in 100% DMSO. Kds were determined using an 11-point 3-fold compound dilution series with three DMSO control points. All compounds for Kd measurements were distributed by acoustic transfer (non-contact dispensing) in 100% DMSO. The compounds were then diluted directly into the assays such that the final concentration of DMSO was 0.9%. All reactions were performed in polypropylene 384-well plate. Each was a final volume of 0.02 ml. The assay plates were incubated at room temperature with shaking for 1 hour and the affinity beads were washed with wash buffer (10 mM HEPES, 50 mM NaCl, 1 mM EDTA, 0.01% Tween 20). The beads were then re-suspended in elution buffer (10 mM HEPES, 50 mM NaCl, 1 mM EDTA, 0.01% Tween 20, 20 mM sodium phenyl phosphate) and incubated at room temperature with shaking for 30 minutes. The SH2 domain concentration in the eluates was measured by qPCR.

Results are shown in Table 2 for STAT1, STAT2, STAT3, STAT4, STAT5a, and STAT5b Kds, where 5 nM≤A<50 nM, 50 nM≤B<100 nM, 100 nM≤C<500 nM, 500 nM≤D<1000 nM, 1000 nM≤E<5000 nM (E* indicates only that the Kd was determined to be >1000 nM), 5000 nM≤F<10000 nM, and G>10000 nM; NT=not tested.

TABLE 2
(STAT1/2/3/4/5a/5b)

| No. | STAT1 | STAT2 | STAT3 | STAT4 | STAT5a | STAT5b |
|---|---|---|---|---|---|---|
| A1 | F | E | F | F | C | C |
| A2 | F | C | E | F | E | C |
| A3 | G | E | F | F | D | C |
| A4 | F | C | E | E | C | C |
| A5 | F | E | F | G | D | D |
| A6 | E | C | D | F | A | A |
| A7 | E* | D | D | E* | A | B |
| A8 | A | C | A | C | A | A |
| A9 | D | E* | D | E* | NT | A |
| A10 | E* | E* | E* | E* | C | C |
| A11 | E* | C | E* | E* | A | A |
| A12 | B | D | A | E* | B | A |
| A13 | E* | E* | E* | E* | A | B |
| A14 | E* | D | E* | E* | C | B |
| A15 | E* | E* | E* | E* | C | C |
| A16 | E* | D | E* | E* | B | C |
| A17 | E* | E* | E* | E* | C | C |
| A18 | E* | C | E* | E* | A | A |
| A19 | E* | C | E* | D | B | B |
| A20 | E* | E* | E* | E* | C | C |
| A21 | G | D | E | F | C | C |
| A22 | E* | D | E* | E* | C | B |
| A23 | E* | D | E* | E* | B | B |
| A24 | E* | E* | D | E* | D | D |
| A25 | E* | D | E* | E* | B | C |
| A26 | E* | E* | F* | E* | C | C |
| A27 | E* | E* | E* | E* | C | C |
| A28 | E* | E* | E* | E* | C | C |
| A29 | E* | E* | E* | E* | D | E* |
| A30 | E* | C | E* | E* | C | C |
| A31 | E* | E* | C | E* | E* | E* |
| A32 | D | E* | E* | E* | C | B |
| A33 | E* | D | E* | E* | C | B |
| A34 | E* | E* | E* | E* | C | C |
| A35 | E* | C | E* | E* | C | C |
| A36 | E* | E* | E* | E* | D | D |
| A37 | E* | E* | E* | E* | B | B |
| A38 | E* | E* | E* | E* | E* | E* |
| A39 | E* | E* | E* | E* | E* | E* |
| A40 | E* | E* | E* | E* | C | C |
| A41 | E* | E* | E* | E* | B | B |
| A42 | E* | D | E* | E* | C | C |
| A43 | E* | E* | E* | E* | C | C |
| A44 | E* | E* | E* | E* | D | D |
| A45 | E* | D | E* | E* | C | C |
| A46 | E* | E* | E* | E* | C | E* |
| A47 | E* | E* | E* | E* | C | C |
| A48 | E* | E* | E* | E* | B | C |
| A49 | E* | E* | E* | E* | C | C |
| A50 | E* | E* | E* | E* | E* | E* |
| A51 | E* | E* | E* | E* | E* | E* |
| A52 | E* | E* | E* | E* | C | D |
| A53 | E* | E* | E* | E* | D | E* |
| A54 | E* | D | E* | E* | B | B |
| A55 | E* | C | E* | E* | B | B |
| A56 | E* | E* | D | E* | B | C |
| A57 | E* | E* | E* | E* | C | C |
| A58 | E* | D | E* | E* | C | C |
| A59 | E* | C | C | E* | C | C |
| A60 | E* | E* | E* | E* | D | D |
| A61 | E* | E* | E* | E* | E* | E* |
| A62 | E* | E* | E* | E* | D | C |
| A63 | E* | E* | E* | E* | C | D |
| A64 | E* | E* | E* | E* | C | C |
| A65 | — | D | D | D | C | C |
| B1 | E* | E* | E* | E* | E* | E* |
| B2 | F | E | G | G | E | E |
| B3 | G | F | G | G | E | E |
| B4 | G | E | G | G | E | E |
| B5 | G | F | G | G | E | E |
| B6 | F | E | G | G | E | E |
| B7 | E* | E* | E* | E* | D | D |
| B8 | G | G | G | G | E | E |
| B9 | E* | E* | E* | E* | C | D |
| B10 | G | E | G | G | G | F |
| B11 | G | E | G | G | C | D |
| B12 | G | F | G | G | E | F |
| B13 | G | G | G | G | G | G |
| B14 | E* | E* | E* | E* | E* | E* |
| B15 | E* | E* | E* | E* | E* | E* |
| B16 | E* | E* | E* | E* | E* | E* |
| B17 | E* | E* | E* | E* | E* | E* |
| B18 | E* | E* | E* | E* | D | D |
| B19 | E* | E* | E* | E* | C | C |
| B20 | E* | E* | E* | E* | E* | E* |
| B21 | E* | E* | E* | E* | E* | E* |
| B22 | E* | E* | E* | E* | E* | E* |
| B23 | E* | E* | E* | E* | E* | E* |
| B24 | E* | E* | E* | E* | D | E* |
| B25 | E* | E* | E* | E* | E* | E* |
| B26 | E* | E* | E* | E* | E* | E* |
| B27 | E* | E* | E* | E* | E* | E* |
| B28 | E* | E* | E* | E* | E* | E* |

Results are shown in Table 3 for STAT6 Kd, where A<0.1 nM; 0.1 nM≤B<0.5 nM; 0.5 nM≤C<1 nM; 1 nM≤D<5 nM; and E≥5 nM.

TABLE 3
(STAT6)

| No. | STAT6 |
|---|---|
| A1 | B |
| A2 | B |
| A3 | A |
| A4 | A |
| A5 | A |
| A6 | A |
| A7 | A |
| A8 | A |
| A9 | B |
| A10 | D |
| A11 | A |
| A12 | B |
| A13 | A |
| A14 | A |
| A15 | B |
| A16 | B |
| A17 | B |
| A18 | A |
| A19 | A |
| A20 | A |
| A21 | A |
| A22 | A |
| A23 | A |
| A24 | C |
| A25 | A |
| A26 | C |
| A27 | B |
| A28 | B |
| A29 | B |
| A30 | A |
| A31 | C |
| A32 | A |
| A33 | A |
| A34 | B |
| A35 | A |
| A36 | A |
| A37 | A |
| A38 | A |
| A39 | B |
| A40 | A |
| A41 | B |
| A42 | A |
| A43 | A |
| A44 | A |
| A45 | B |
| A46 | B |
| A47 | A |
| A48 | A |
| A49 | A |
| A50 | A |
| A51 | B |
| A52 | A |
| A53 | A |
| A54 | B |
| A55 | A |
| A56 | B |
| A57 | A |
| A58 | A |
| A59 | A |
| A60 | A |
| A61 | A |
| A62 | B |
| A63 | B |
| A64 | B |
| A65 | A |
| B1 | C |
| B2 | B |
| B3 | B |
| B4 | B |
| B5 | B |
| B6 | B |
| B7 | B |
| B8 | D |
| B9 | C |
| B10 | D |
| B11 | B |
| B12 | C |
| B13 | E |
| B14 | B |
| B15 | E |
| B16 | B |
| B17 | D |
| B18 | B |
| B19 | B |
| B20 | E |
| B21 | E |
| B22 | D |
| B23 | D |
| B24 | D |
| B25 | C |
| B26 | D |
| B27 | B |
| B28 | E |

### Example 3: MSD-pSTAT-PBMC Assay

Materials: Cryopreserved Peripheral Blood Mononuclear Cells (PBMC) are from STEM CELL. Recombinant Human IL-4 is from Peprotech. Rabbit monoclonal Anti-pY641-STAT6 antibody, and lysis buffer are from Cell Signaling Technology (CST). Mouse monoclonal anti-STAT6 antibody is from BioLegend. Assay plates, blocker, and anti-rabbit secondary antibody are from Meso Scale Discovery (MSD).

Assay Method: Cryopreserved PBMCs were thawed out and allowed to recover overnight in IMDM+10% heat-inactivated FBS prior to plating 30,000 (STAT6) cells per well in 96-well U-bottom tissue culture plates. Cells were treated with compound for 3 hrs or 20 hrs, then stimulated with 1 ng/mL IL-4 (STAT6) for 10 min. Cells were then spun down and washed with ice-cold PBS prior to lysing the cell pellet with 1× lysis buffer (CST) with 1×HALT protease and phosphatase inhibitor cocktail (Thermo). Lysates were freeze-thawed and then transferred to and incubated overnight at 4° C. with shaking in QuickPlex 96-well high bind assay plates (MSD) pre-coated overnight with 30 μL per well of 2 μg/mL mouse monoclonal anti-STAT6 antibody (STAT6) in 1×PBS, and pre-blocked for 1 hour with 3% Blocker-A (MSD). Captured protein in the assay plates were then washed and probed with 25 μL per well of 0.18 μg/ml rabbit monoclonal Anti-pY641-STAT6 antibody (STAT6) in 1% Blocker-A for 1 hour at room temperature with shaking, then washed and probed with 25 ul per well of 1 ug/ml Sulfo-TAG Labeled Goat Anti-Rabbit Antibody (MSD) in 1% Blocker-A for 1 hour at room temperature with shaking. Assay plates were then washed and 150 μL of 1× Read Buffer-T (MSD) was added to each well prior to reading on an SQ120 MSD Plate Reader. Assay signal from each sample was subtracted by the signal from unstimulated control wells and normalized to DMSO control wells. IC₅₀ values were calculated using Graph Pad Prism Dose-Response Nonlinear Regression with variable slope. Results are shown in Table 4. For pSTAT6: A<1 nM; 1 nM≤B<5 nM; 5 nM≤C<10 nM; 10 nM≤D<50 nM; and E≥50 nM.

TABLE 4
(pSTAT6)

| No. | pSTAT6 |
|---|---|
| C1 | B |
| C2 | B |
| C3 | B |
| C4 | A |
| C5 | A |
| C6 | A |
| C7 | A |
| C8 | A |
| C9 | B |
| C10 | A |
| C11 | B |
| C12 | B |
| C13 | A |
| C14 | B |
| C15 | B |
| C16 | B |
| C17 | A |
| C18 | B |
| C19 | A |
| C20 | B |
| C21 | D |
| C22 | C |
| C23 | A |
| C24 | B |
| C25 | A |
| C26 | B |
| C27 | C |
| C28 | B |
| C29 | C |
| C30 | D |
| C31 | D |
| C32 | B |
| C33 | D |
| C34 | B |
| C35 | D |
| C36 | C |
| C37 | B |
| C38 | B |
| C39 | C |
| C40 | B |
| C41 | C |
| C42 | D |
| C43 | E |
| C44 | D |
| C45 | D |
| C46 | D |
| C47 | D |
| C48 | E |
| C49 | D |
| C50 | B |
| C51 | D |
| C52 | B |
| C53 | A |
| C54 | A |
| C55 | B |
| C56 | B |
| C57 | B |
| C59 | B |
| C60 | B |
| C61 | A |
| C62 | B |
| C63 | A |
| C64 | A |
| C65 | A |
| C66 | A |
| C67 | B |
| C68 | A |
| C69 | B |
| C70 | A |
| C71 | A |
| C72 | B |
| C73 | A |
| C74 | A |
| C75 | A |
| C76 | B |
| C77 | B |
| C78 | B |
| C79 | B |
| C80 | B |
| C81 | B |
| C82 | A |
| C83 | A |
| C84 | A |
| C85 | B |
| C86 | B |
| C87 | B |
| C88 | B |
| C89 | A |
| C90 | A |
| C91 | A |
| C92 | A |
| C93 | A |
| C94 | A |
| C95 | B |
| C105 | A |
| C106 | A |
| C107 | A |
| C108 | B |
| C109 | C |
| C110 | A |
| C111 | A |
| C112 | B |
| C113 | B |
| C114 | A |
| C115 | B |
| C116 | A |
| C117 | B |
| C118 | A |
| C119 | B |
| C120 | B |
| C121 | A |
| C122 | A |
| C123 | A |
| C124 | A |
| C125 | A |
| C126 | A |
| C127 | A |
| C128 | A |
| C129 | B |
| C130 | B |
| C131 | B |
| C132 | A |
| C133 | A |
| C134 | B |
| C135 | A |
| C136 | A |
| C137 | A |
| C138 | B |
| C139 | B |
| C140 | A |
| C141 | A |
| C142 | B |
| C143 | A |
| C144 | A |
| C145 | C |
| C146 | B |
| C147 | B |
| C148 | A |
| C149 | B |
| C150 | B |
| C151 | B |
| C152 | E |
| C153 | E |
| C154 | B |
| C155 | A |
| C156 | A |
| C157 | B |
| C158 | B |
| C159 | B |
| C160 | A |
| C161 | B |
| C162 | A |
| C167 | B |
| C168 | D |
| C169 | C |
| C170 | A |
| C172 | B |
| C173 | A |
| C174 | B |
| C175 | A |
| C176 | A |
| C177 | A |
| C178 | B |
| C179 | B |
| C180 | A |
| C181 | C |
| C182 | A |
| C183 | B |
| C184 | B |
| C185 | A |
| C186 | A |
| C187 | B |
| C188 | A |
| C189 | A |
| C190 | C |
| C191 | B |
| C192 | B |
| C193 | B |
| C194 | A |
| C195 | A |
| C196 | B |
| C197 | A |
| C198 | A |
| C199 | B |
| C200 | B |
| C201 | B |
| C202 | B |
| C203 | B |
| C204 | C |
| C205 | A |
| C206 | E |
| C207 | E |
| C208 | A |
| C209 | A |
| C210 | B |
| C211 | C |
| C212 | B |
| C213 | B |
| C214 | B |
| C215 | B |

### Example 4: Flow Cytometry-pSTAT6-Whole Blood Assay

Materials: Recombinant canine IL-4 is from Novus Biologics. PE anti-STAT6 Phospho (Tyr641) Antibody and Human TruStain FcX are from BioLegend. BD Phosflow Lyse/Fix Buffer 5× and BD Phosflow Perm Buffer III are from BD Biosciences.

Assay Method: Whole blood is serially collected into K₂EDTA tubes from beagle dogs via the cephalic or saphenous vein at various times pre- and post-dosing (P.O.) and stored on wet ice until the following protocol can be performed. 25 μL of each whole blood sample is stimulated with 1 ng/mL IL-4 for 15 min. Each sample then receives 500 μL of 1× Lyse/Fix Buffer, which is prepared by adding 4 volumes of water to 1 volume of 5× Lyse/Fix Buffer, and is incubated at 37° C. for 10 min. Samples are then spun down and washed with PBS prior to addition of 200 μL Perm Buffer III and incubation on ice for 30 min. Samples are then spun down and washed twice with PBS. Each sample is then stained with 50 μL of PBS containing 5 μL of Human TruStain FcX and incubated on ice for 15 min. Each sample then receives 50 μL of PBS containing 5 μL of PE anti-STAT6 Phospho (Tyr641) Antibody and is incubated at room temperature in the dark for 30 min. Samples are spun down and washed with PBS prior to final resuspension in 100 μL of PBS. Samples are analyzed on a flow cytometer capable of detecting signal from the PE fluorophore. Cells expressing pSTAT6 signal are determined by gating relative to a sample-matched unstimulated (no IL-4) control. Inhibition of pSTAT6 is determined by subtracting the percentage of pSTAT6 positive cells in each sample divided by the percentage of pSTAT6 positive cells in the animal-matched pre-dose control sample from 100. Additional normalization to a vehicle-matched control may be applied, such that the average inhibition of vehicle control samples at each individual timepoint is equal to 0%.

### Example 5: PBMC PK Assay

Assay Method: Up to 5 mL whole blood is serially collected into K₂EDTA tubes from beagle dogs via the cephalic or saphenous vein at various times pre- and post-dosing (P.O.) and stored on wet ice until the following protocol can be performed. Each whole blood sample is diluted with 10 volumes of RBC Lysis Buffer and incubated on ice for 10 min with mixing. Samples are quenched with ice-cold PBS, spun down at 4° C., and washed again with ice-cold PBS prior to resuspension in 1 mL ice-cold PBS. PBMC counts are determined, and samples are spun down at 4° C. Supernatants are removed from each cell pellet and a normalized volume of 70% methanol containing 1% formic acid is added to each sample based on the lowest cell count within the experiment, such that the sample with the lowest cell count is resuspended in 100 μL of the 70% methanol/1% formic acid solution. Samples are vortexed for 1-2 min and stored as a mixture at −80° C. until LC-MS/MS analysis can be performed. Upon LC-MS/MS analysis, samples are spun down and, once pelleted, 20-30 μL of supernatant is added to 200 μL of acetonitrile containing 1% formic acid and 40 ng/mL diclofenac internal standard. The samples are vortexed for 1 minute, spun down, and 100 μL is transferred to a new plate. The samples are then injected for LC-MS/MS analysis. The amount of analyte (parent drug) in each sample is determined by comparing the detected peak counts normalized to the internal standard to a standard curve generated from known amounts of analyte. Intracellular PBMC concentrations are determined by calculating the mass of analyte detected in each cell pellet divided by the volume of cells within that pellet, assuming an individual cell volume of 2E-13 L. PBMC PK averages are calculated by averaging the intracellular PBMC PK concentrations at individual timepoints collected over a 24-hour period. PBMC PK AUCs are determined by multiplying the PBMC PK average by 24 to approximate the area under the curve over a full 24-hour period.

### Example 6: CD23 Expression and TARC Production

Assay Method: Frozen human PBMCs are thawed and rested overnight at 37° C., 5% CO₂. The following day 50,000 cells are plated in 96-well dishes, dosed with diluting concentrations of test article (1,000-10,000 nM top concentration; 1:3-1:5 dilution) and stimulated with 10 ng/mL IL-4 and 10 μg/mL anti-IFN-γ or 1 ng/mL IL-13. For assessment of the induction of CD23 surface expression, cells are collected after 4 days of incubation at 37° C., 5% CO₂ and evaluated by flow cytometry to determine the CD23 mean fluorescence intensity on CD23 positive cells. For assessment of TARC production, supernatants are collected after 4 days of incubation at 37° C., 5% CO₂ and amounts of TARC protein are measured by electrochemiluminescence (Meso Scale discovery).

1. A compound of Formula (I), 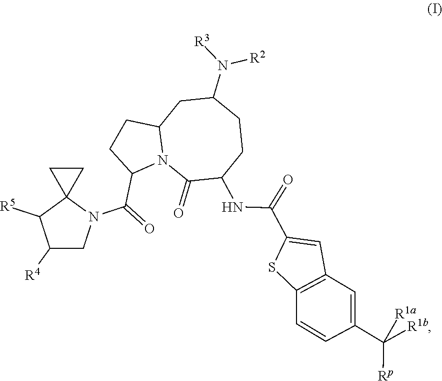
or a pharmaceutically acceptable salt thereof, wherein:
R^{1a} and R^{1b} are each independently H or halogen;
R² is H, (C₁-C₆)alkyl, or (C₃-C₆)cycloalkyl;
R³ is (C₁-C₆)alkyl, halo(C₁-C₆)alkyl, (C₃-C₆)cycloalkyl, 3- to 7-membered heterocyclyl, —(C₁-C₆)alkylene-(C₃-C₆)cycloalkyl, or —(C₁-C₆)alkylene-(3- to 7-membered heterocyclyl), wherein (C₁-C₆)alkyl, (C₃-C₆)cycloalkyl, (C₃-C₆)cycloalkyl of —(C₁-C₆)alkylene-(C₃-C₆)cycloalkyl, or (3- to 7-membered heterocyclyl) of —(C₁-C₆)alkylene-(3- to 7-membered heterocyclyl) is each substituted with 0, 1, 2, 3, 4, or 5 substituents each independently selected from the group consisting of (C₁-C₆)alkyl, (C₁-C₆)alkoxy, cyano, halogen, halo(C₁-C₆)alkyl, and halo(C₁-C₆)alkoxy, or R² and R³ together with the nitrogen atom to which they are attached form 5- to 7-membered heterocyclyl substituted with 0, 1, 2, or 3 substituents each independently selected from the group consisting of (C₁-C₆)alkyl, (C₁-C₆)alkoxy, cyano, halogen, halo(C₁-C₆)alkyl, and halo(C₁-C₆)alkoxy;
R⁴ is H, (C₆-C₁₀)aryl, or 5- to 10-membered heteroaryl, wherein (C₆-C₁₀)aryl or 5- to 10-membered heteroaryl is each substituted with 0, 1, 2, or 3 substituents each independently selected from the group consisting of halogen, cyano, (C₁-C₆)alkyl, (C₁-C₆)alkoxy, halo(C₁-C₆)alkyl, halo(C₁-C₆)alkoxy, and —NR^{4a}R^{4b}, wherein:
each R^{4a} and R^{4b} is independently H or (C₁-C₆)alkyl;
R⁵ is H, cyano, or (C₁-C₆)alkoxy;
R^{P} is 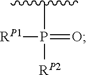
R^{P1} and R^{P2} are each independently —OH, —OR^{Pa}, N-linked amino acid, N-linked amino acid ester, or 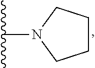 [SMILES: *N1CCCC1]
wherein 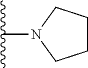 [SMILES: *N1CCCC1]
is substituted with 0, 1, 2, or 3 substituents each independently selected from the group consisting of halo, oxo, COOH, and —C(O)OR^{PE};
each R^{Pa} is independently (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy; and
each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C1-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.

2. The compound of claim 1, or a pharmaceutically acceptable salt thereof, wherein
R^{P1} is —OR^{Pa} and R^{P2} is N-linked amino acid ester, wherein:
N-linked amino acid ester is —NR^{Paa1}CR^{Paa2}R^{Paa3}C(O)OR^{Paa4};
R^{Paa1} is H or (C₁-C₆)alkyl;
R^{Paa2} is H or (C₁-C₆)alkyl, and R^{Paa3} is H, (C₁-C₆)alkyl, —(C₁-C₆)alkylene-(C₁-C₆)alkoxy, or —(C₁-C₆)alkylene-(C₆-C₁₀)aryl; or R^{Paa2} and R^{Paa3}, together with the carbon atom to which they are attached form (C₃-C₆)cycloalkyl; and
R^{Paa4} is (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C5-C8)spirocycloalkyl, or (C₃-C₇)cycloalkyl of —(C₁-C₆)alkylene-(C₃-C₇)cycloalkyl is each substituted with 0, 1, 2, or 3 independently selected halogen.

3. The compound of claim 1, wherein the compound is selected from the group consisting of: 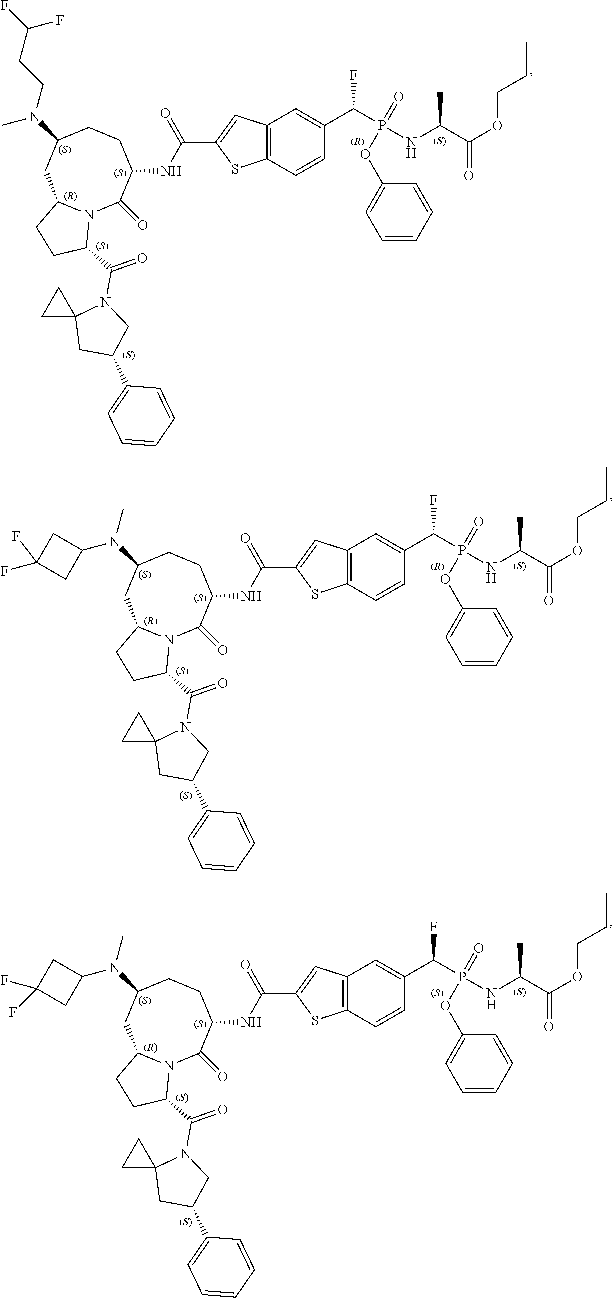 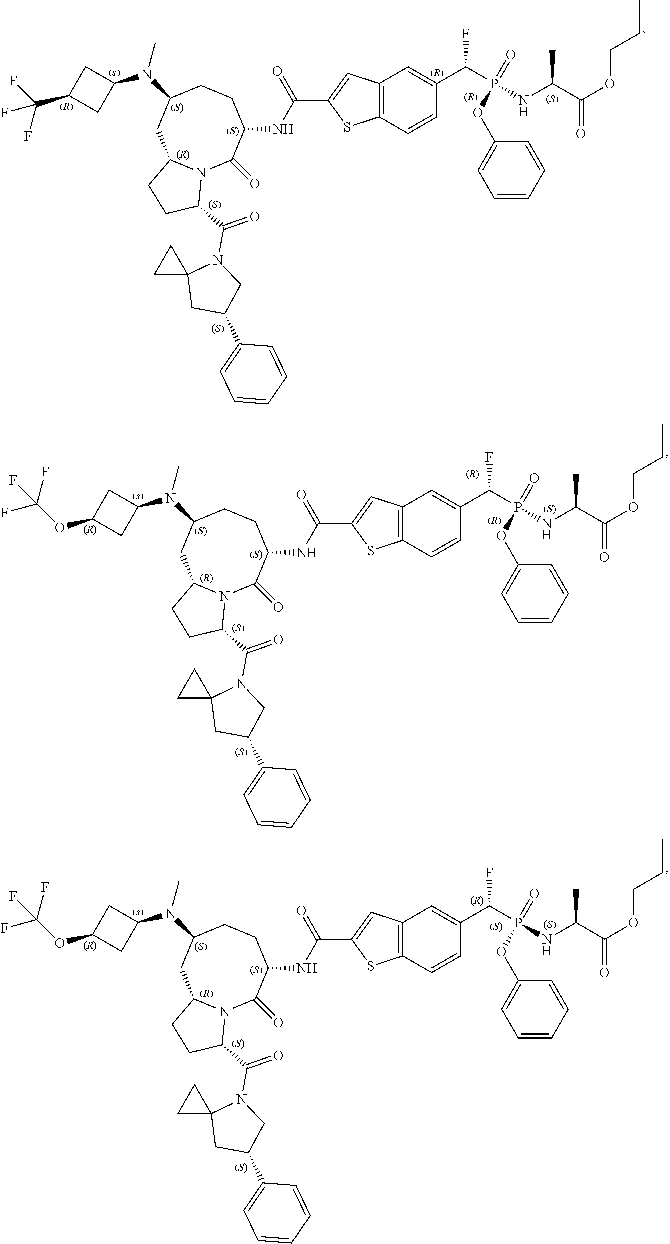 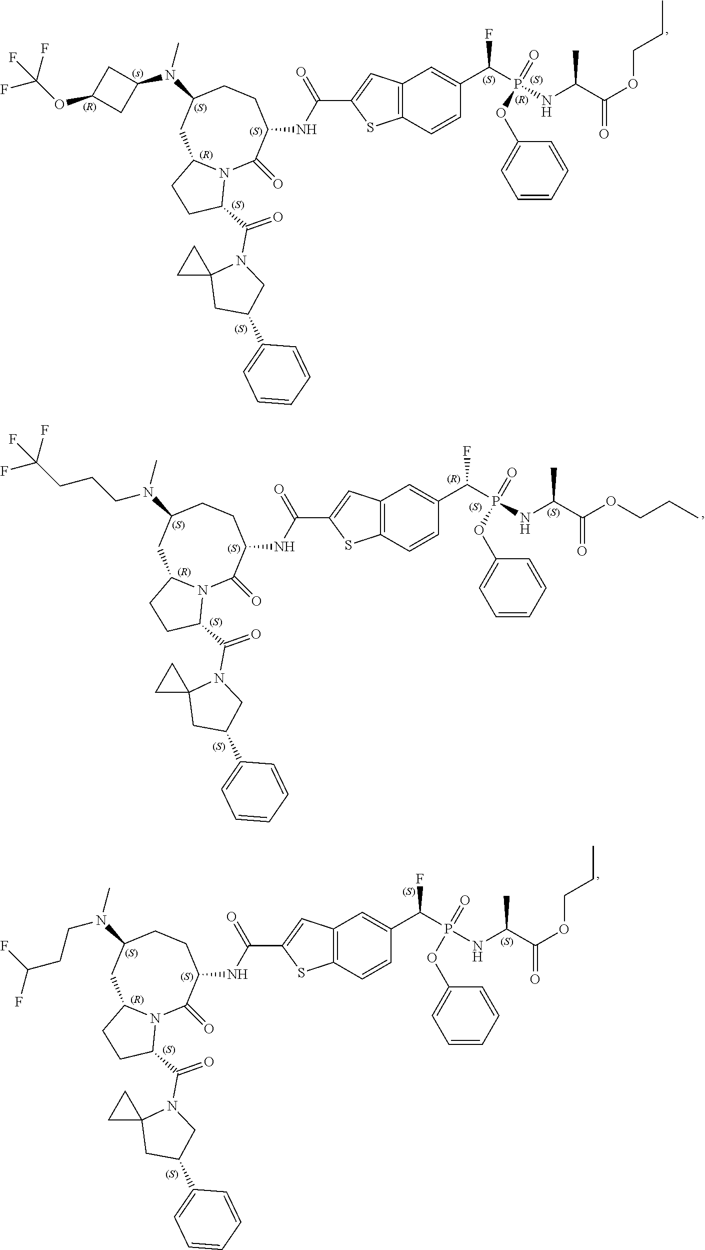 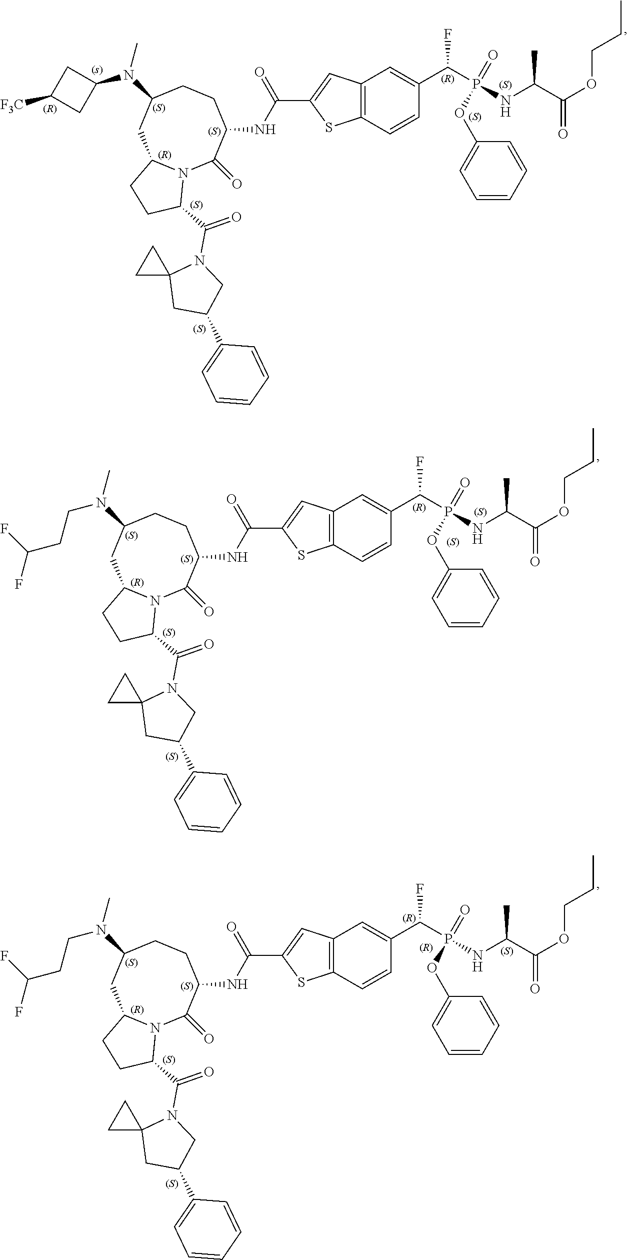 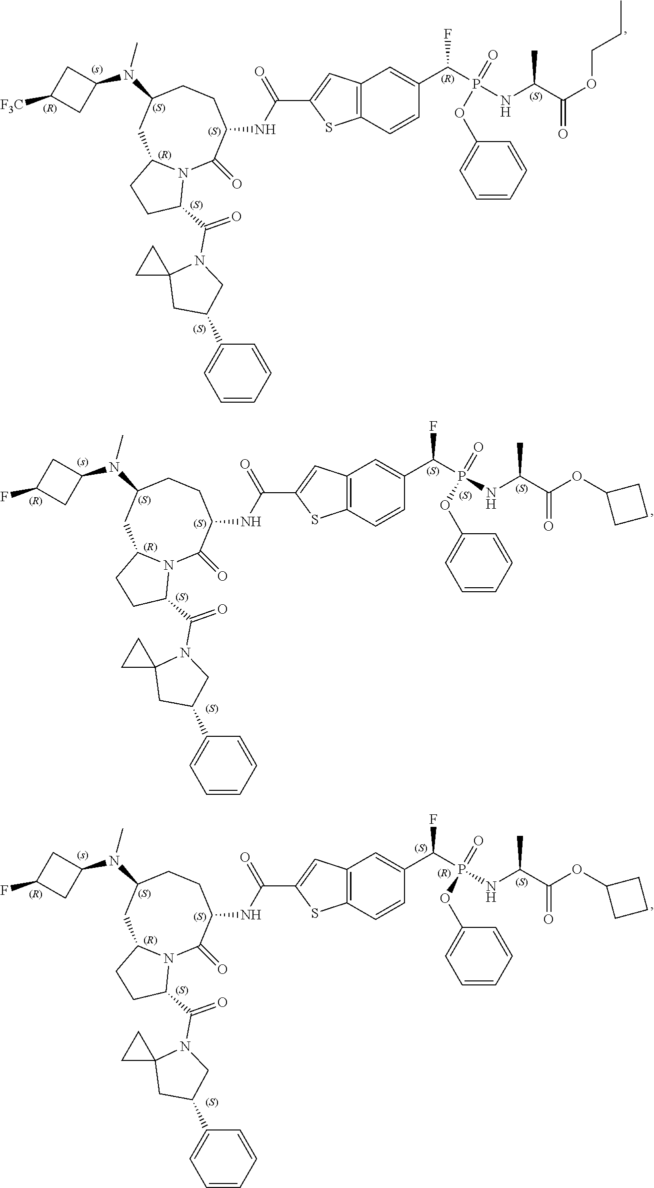 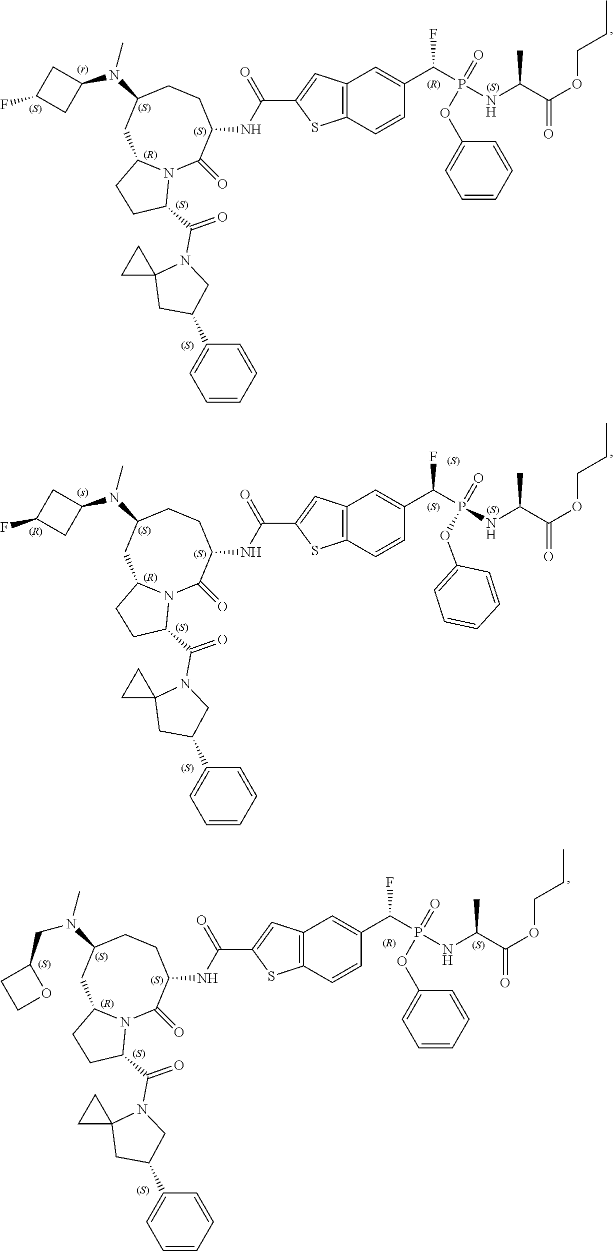 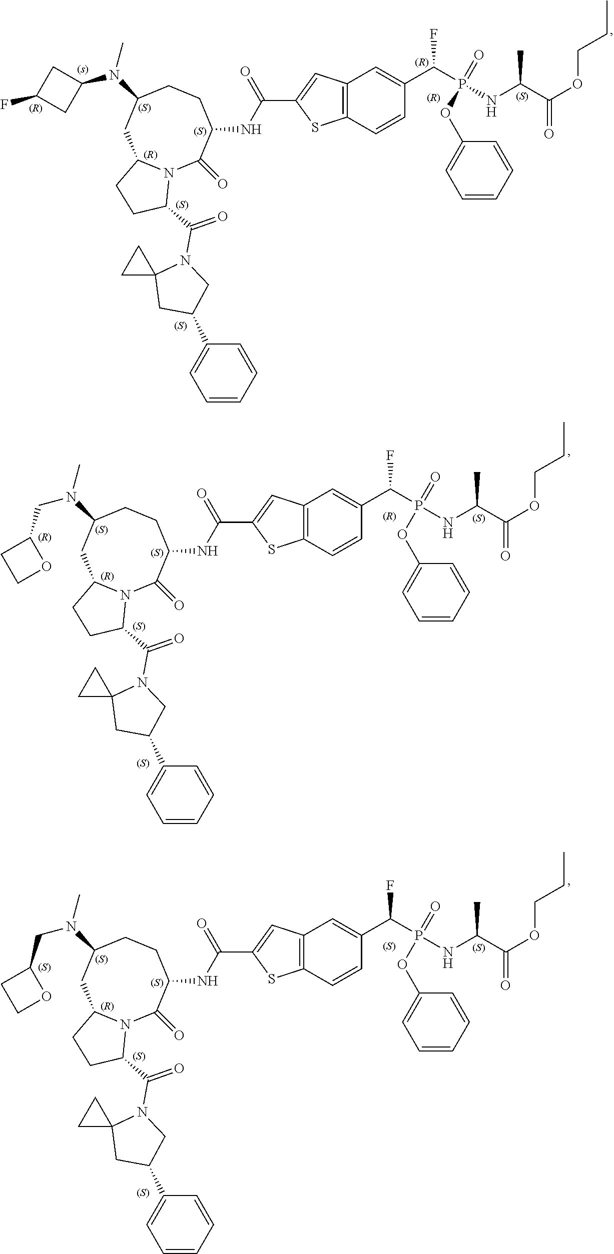 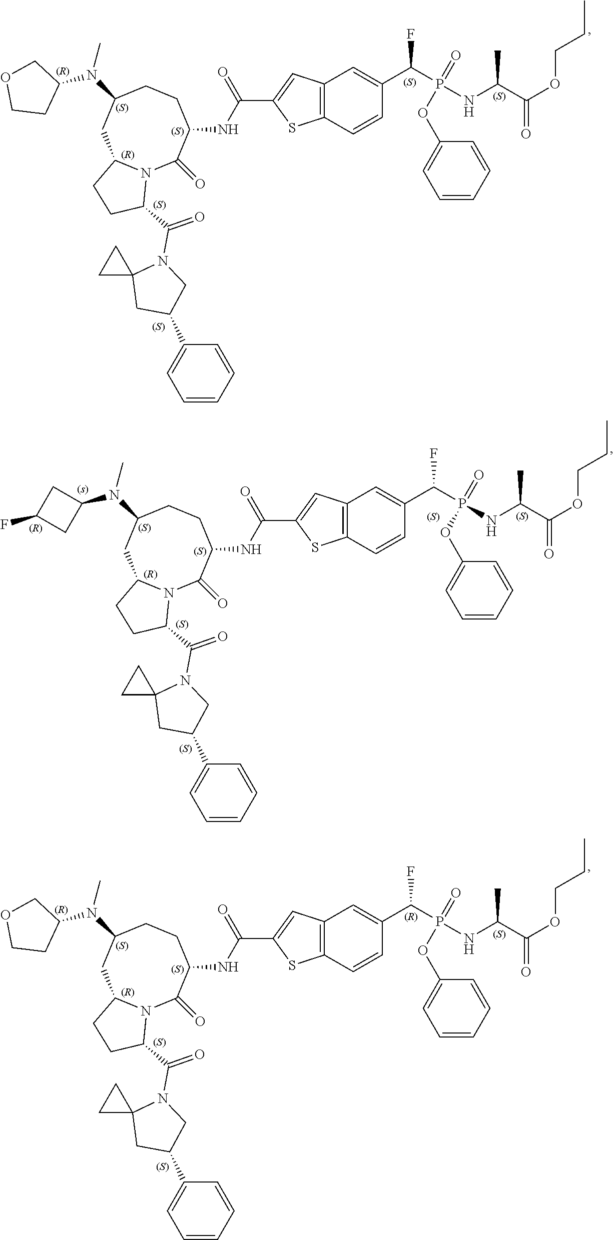 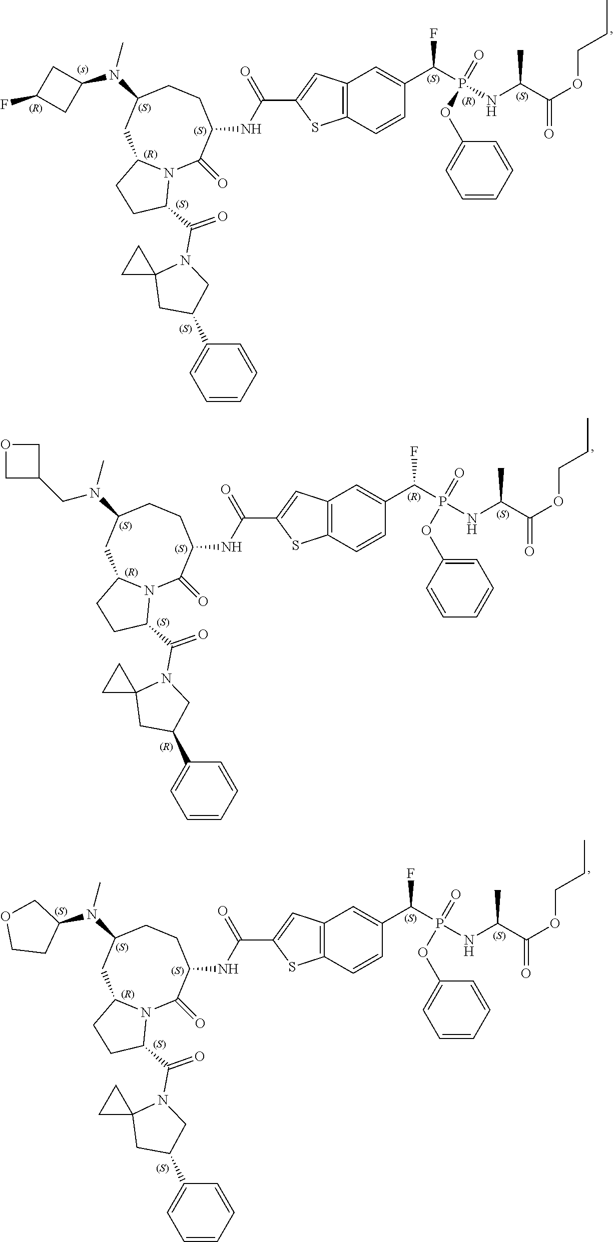 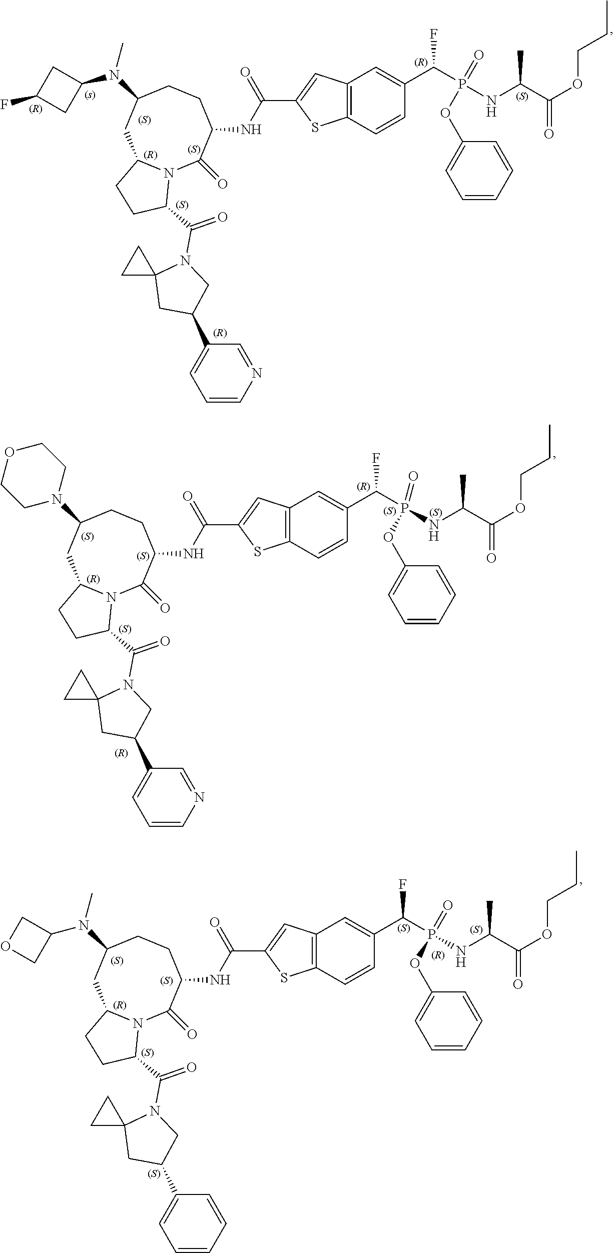 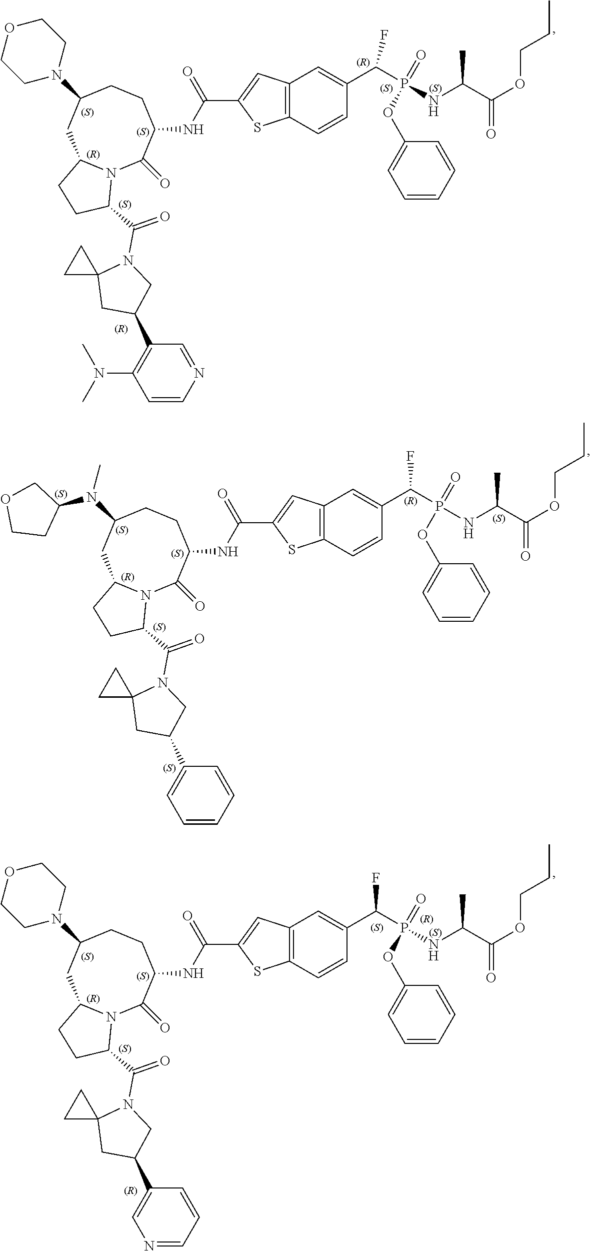 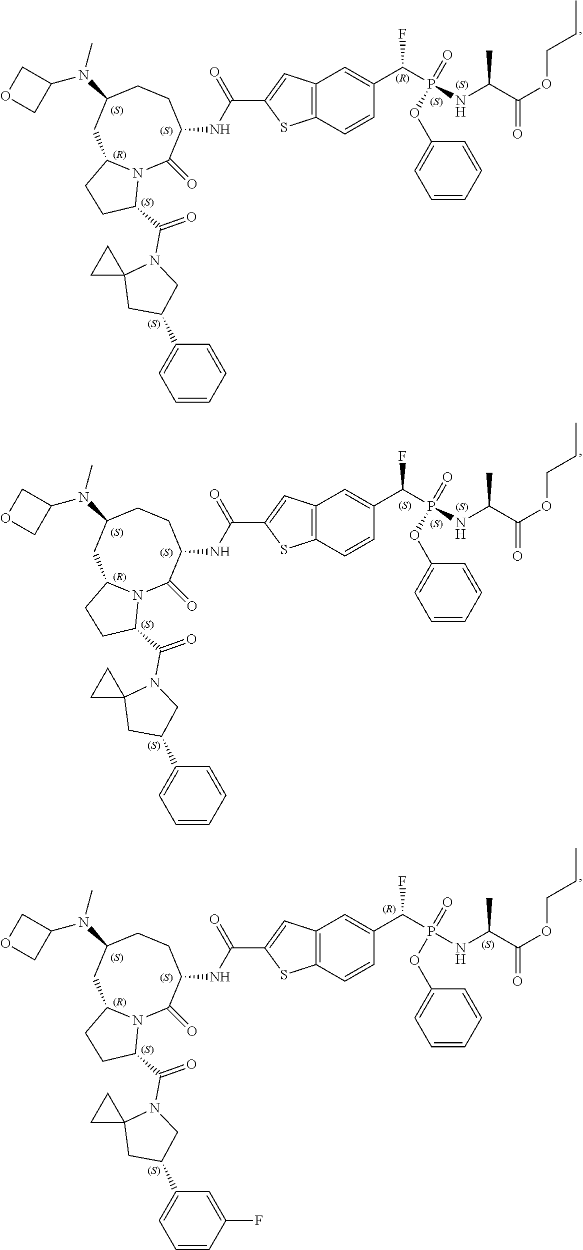 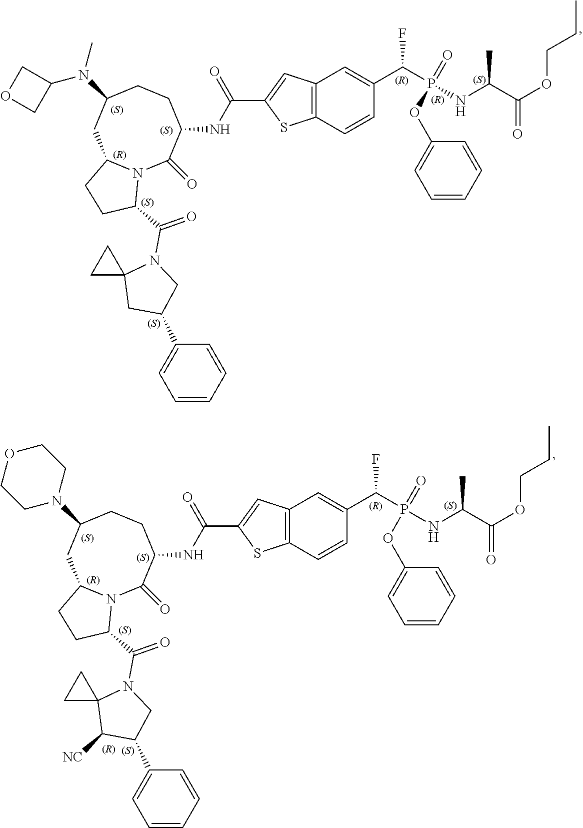 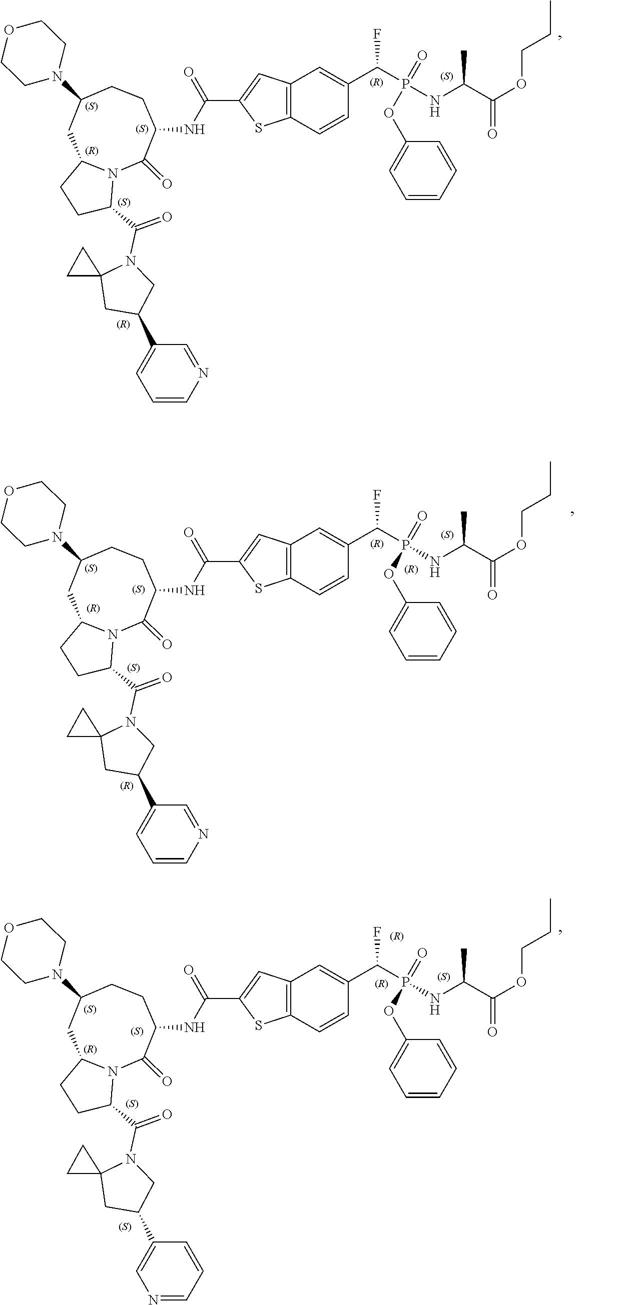 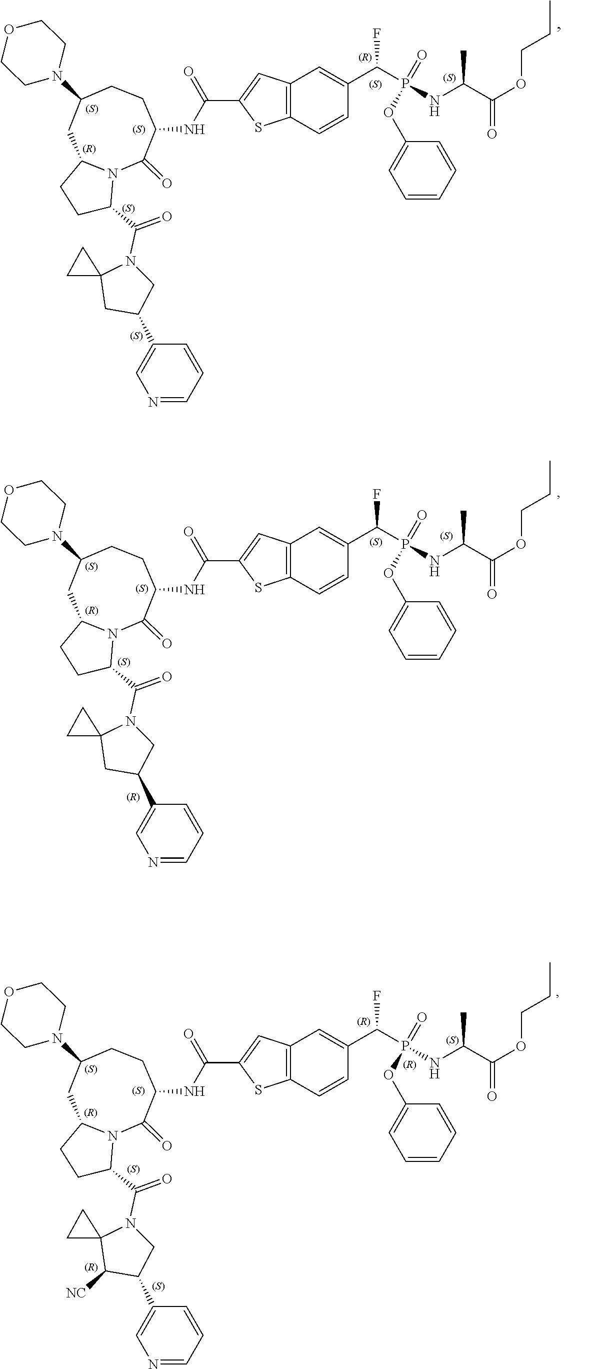 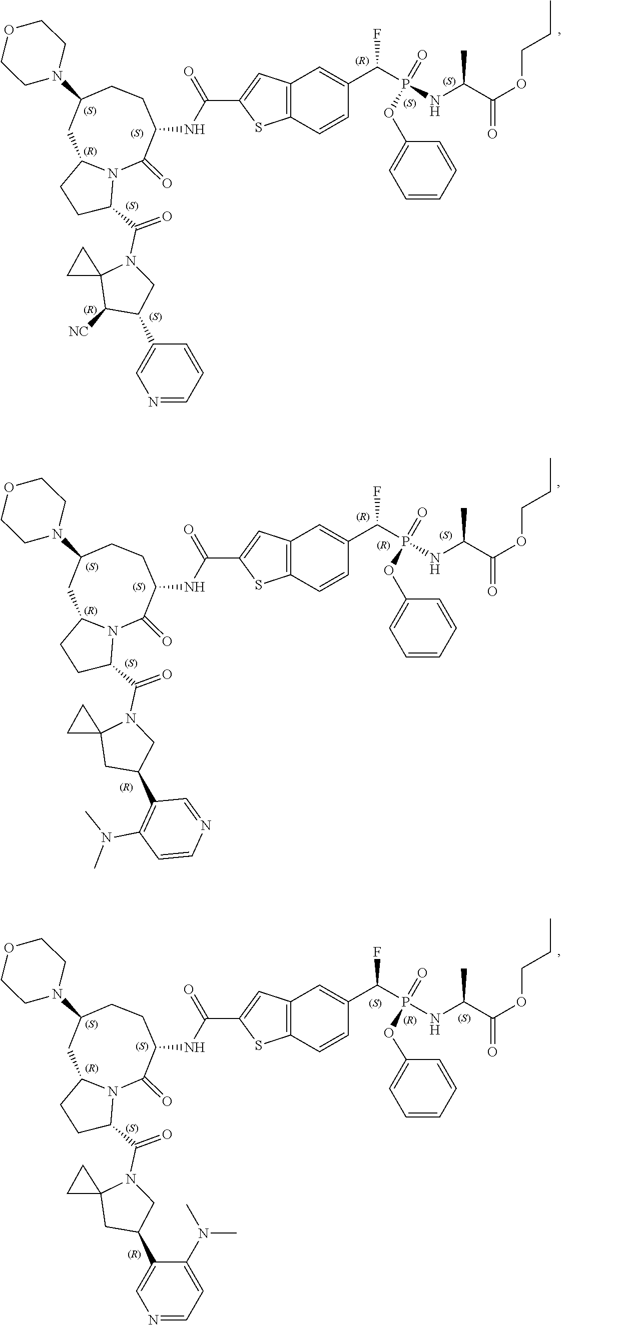 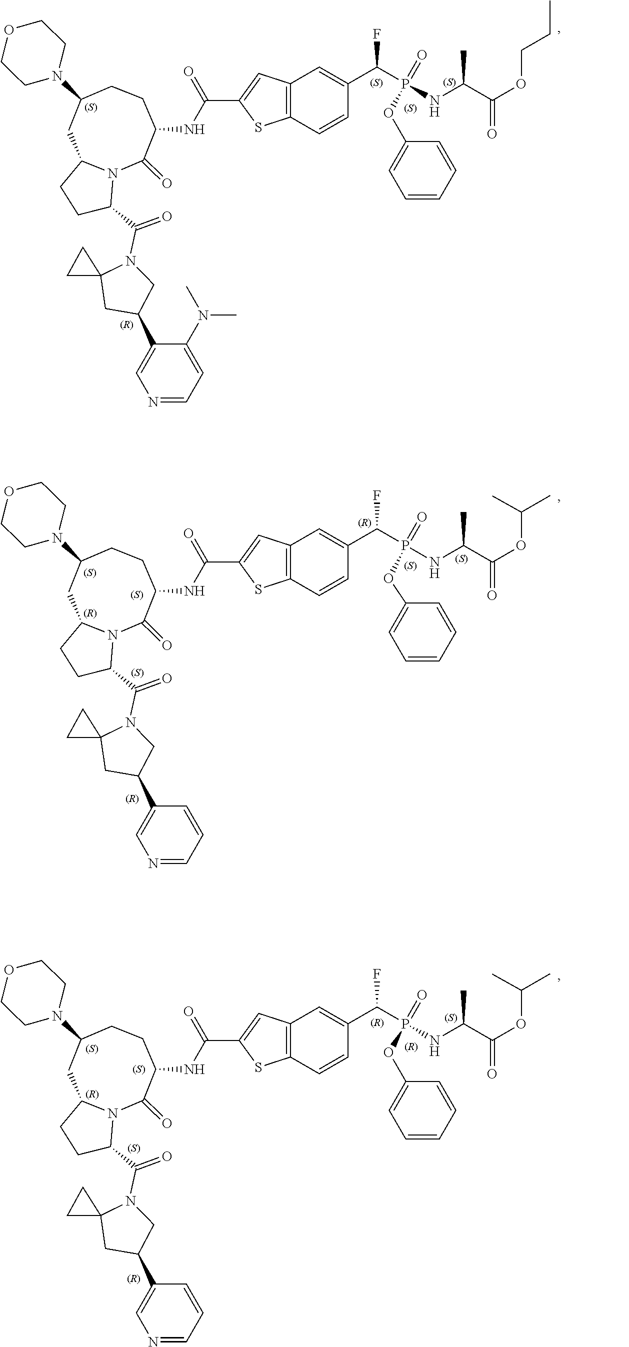 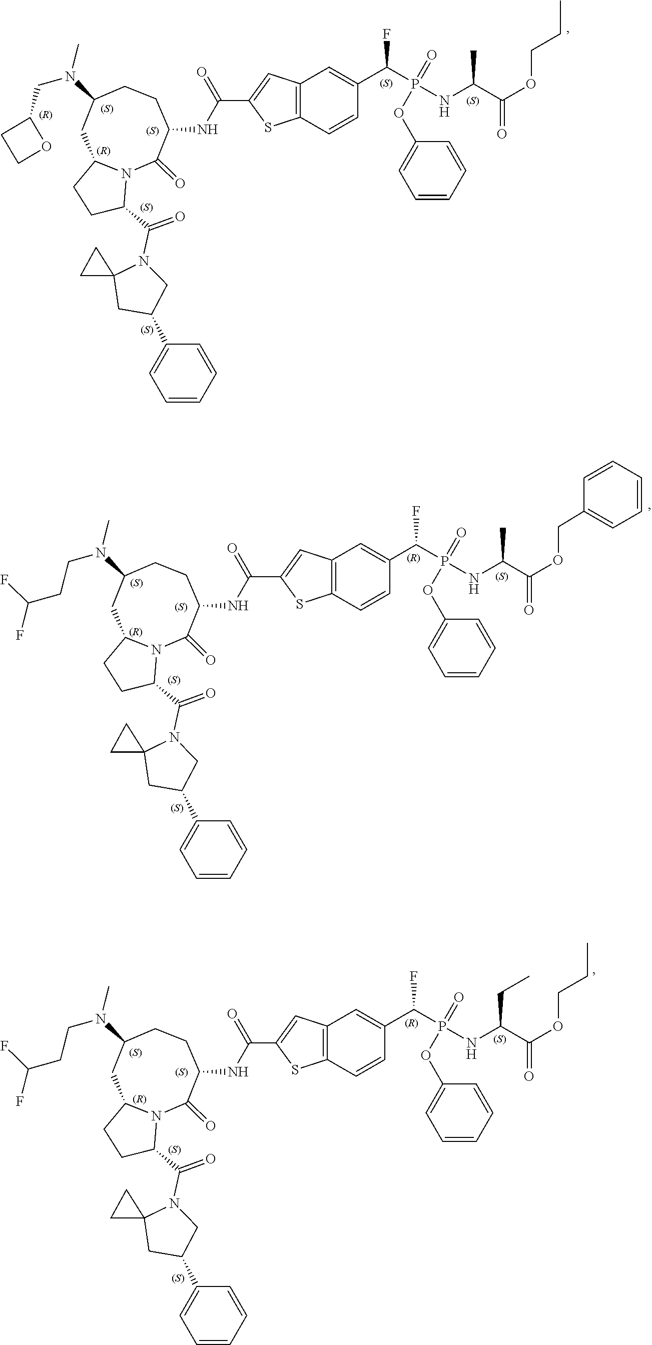 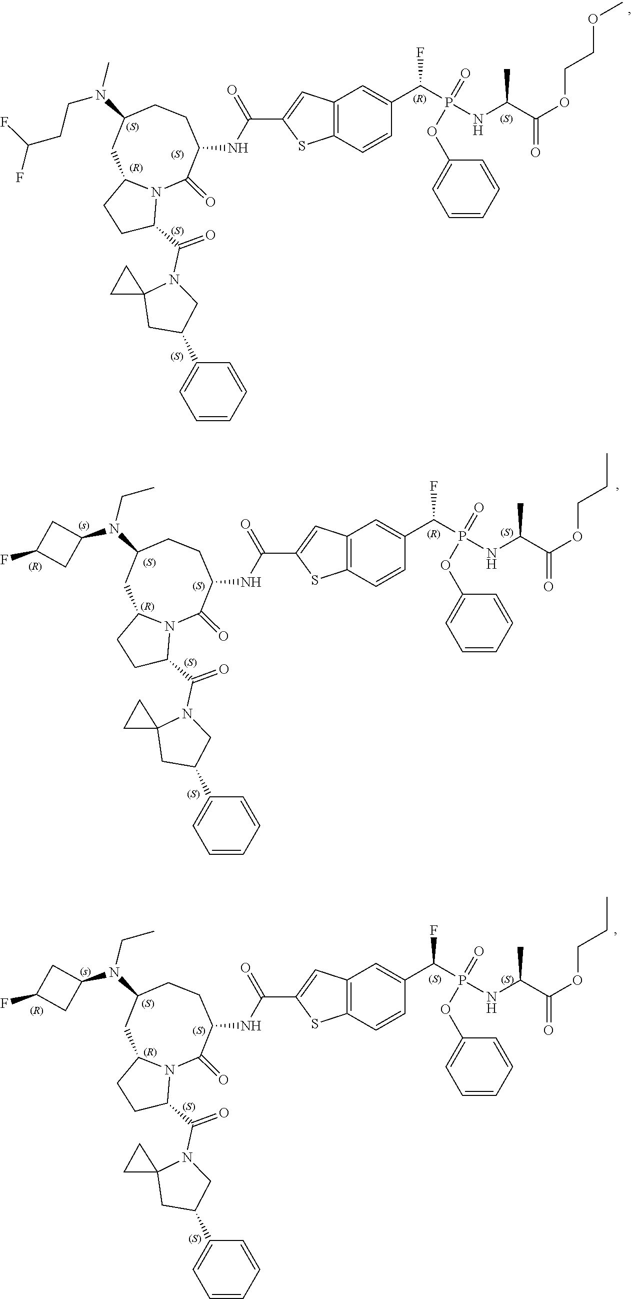 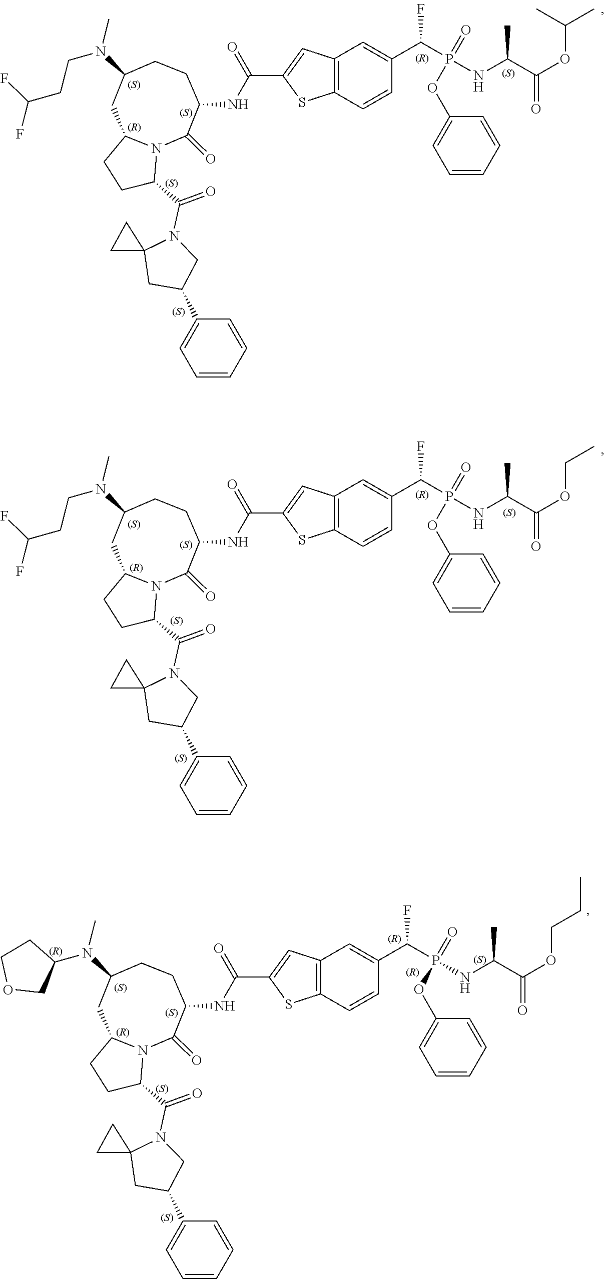 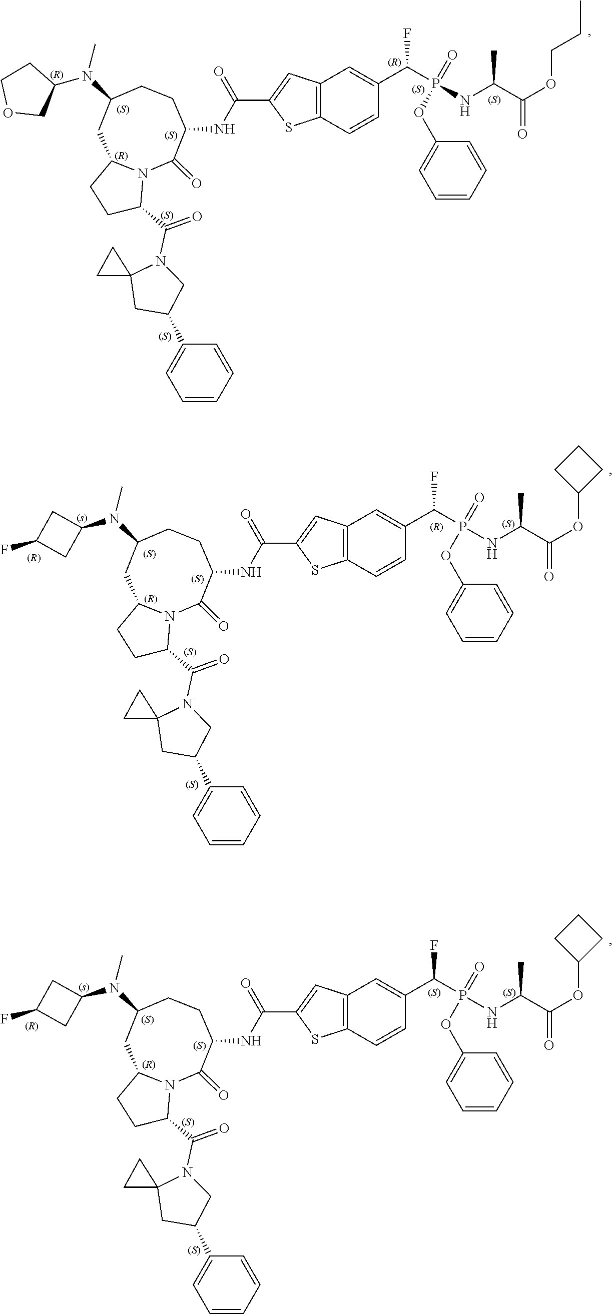 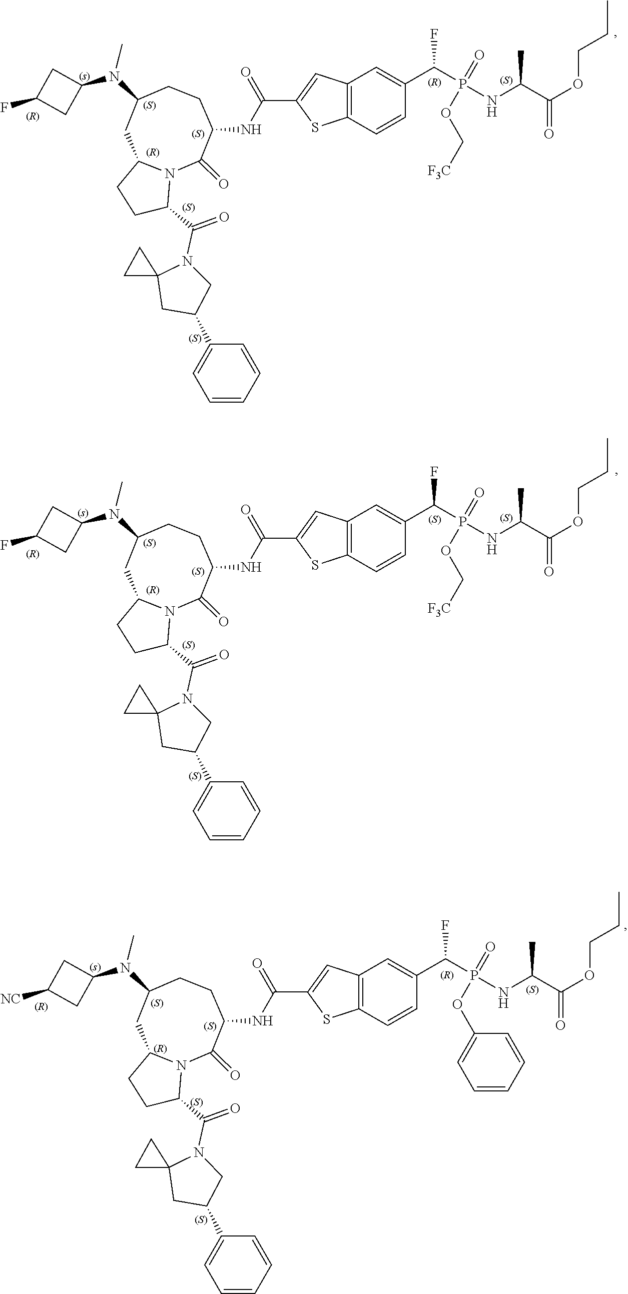 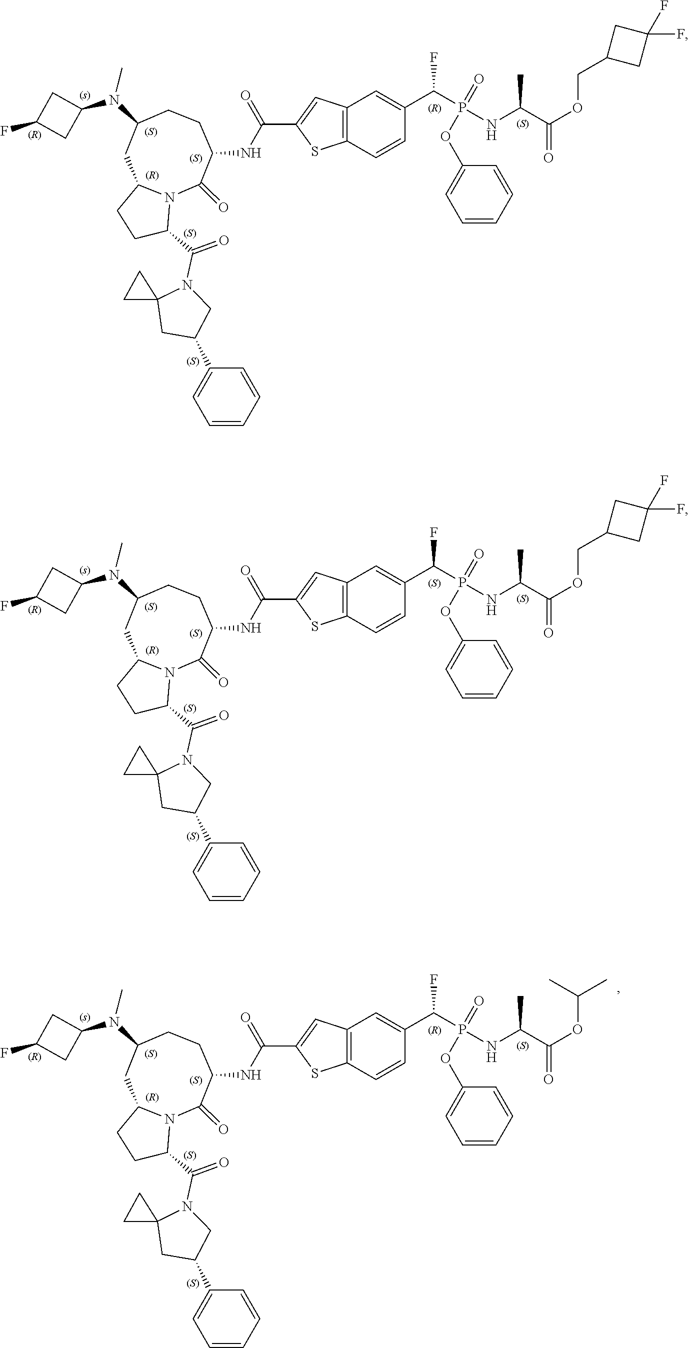 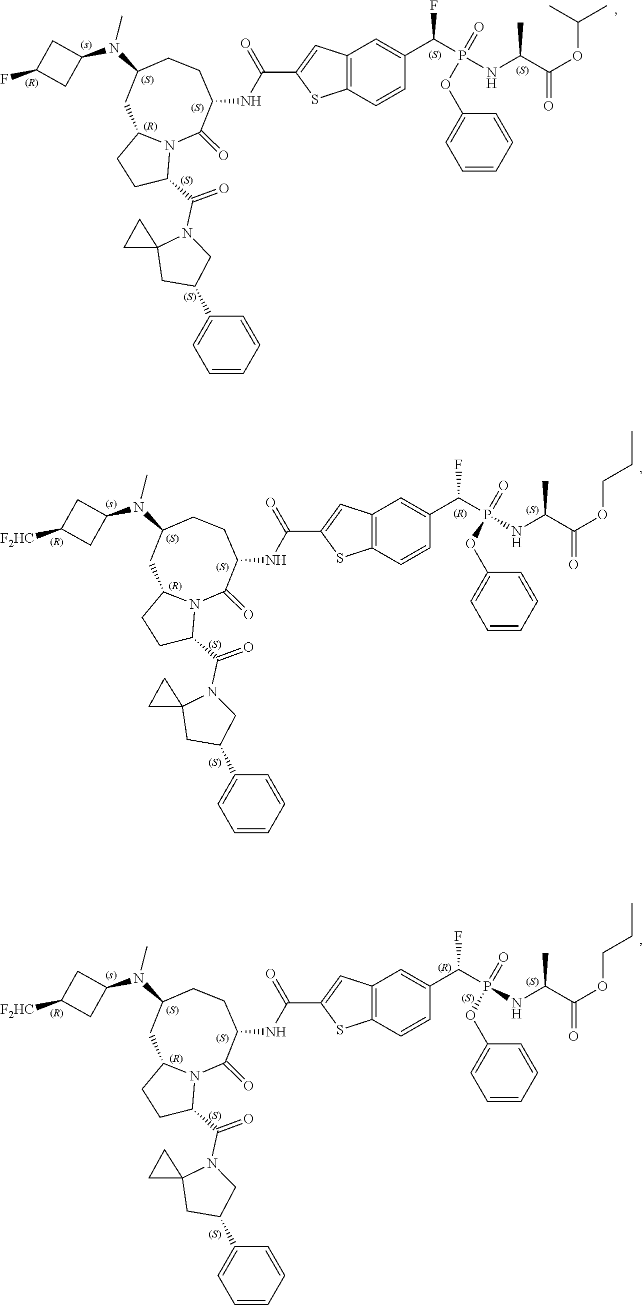 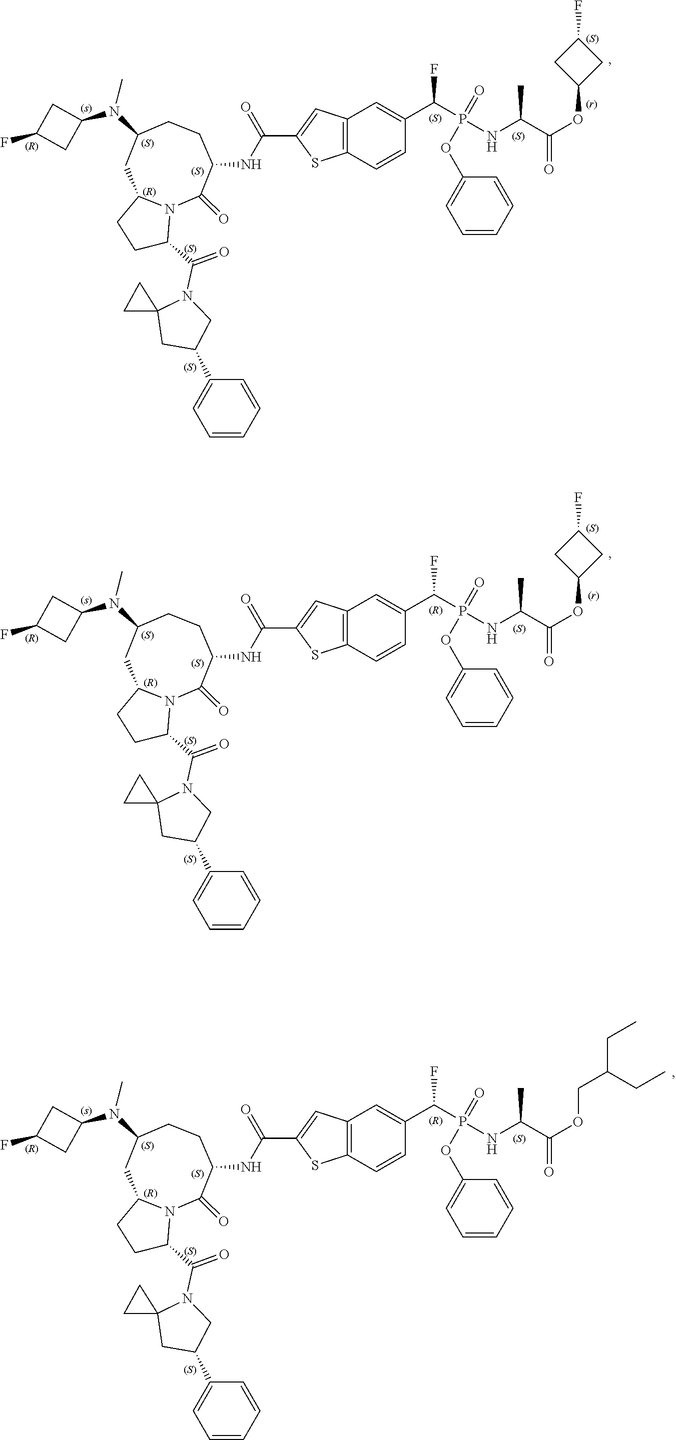 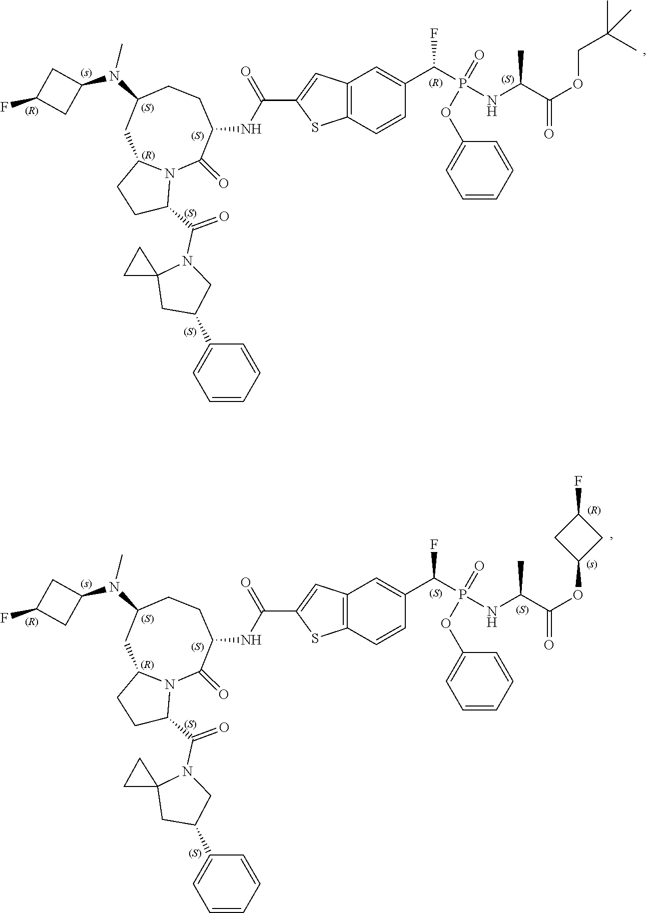 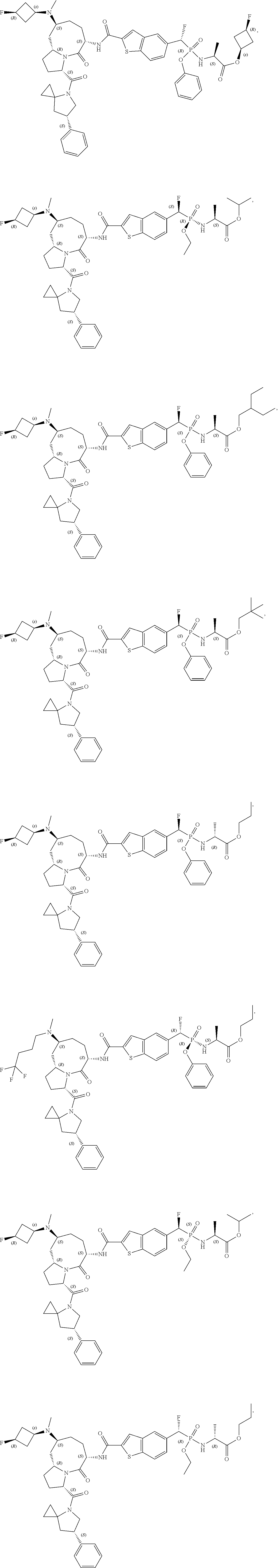 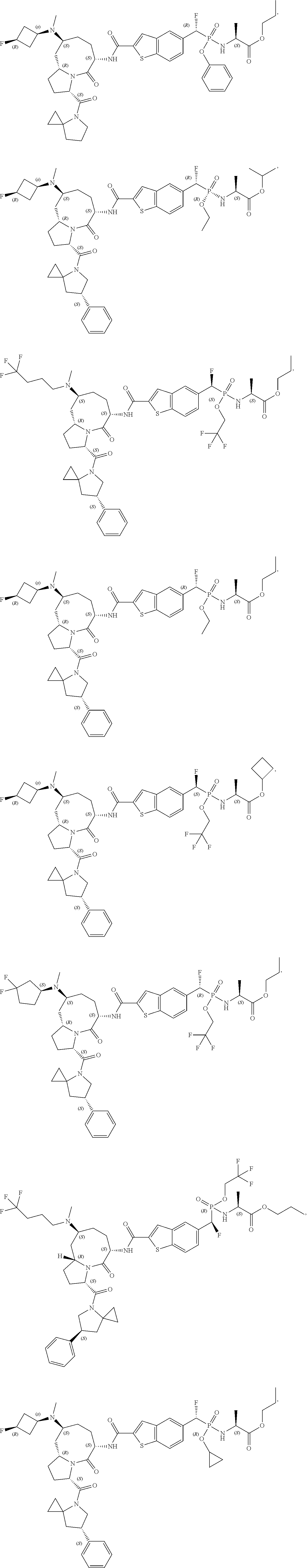 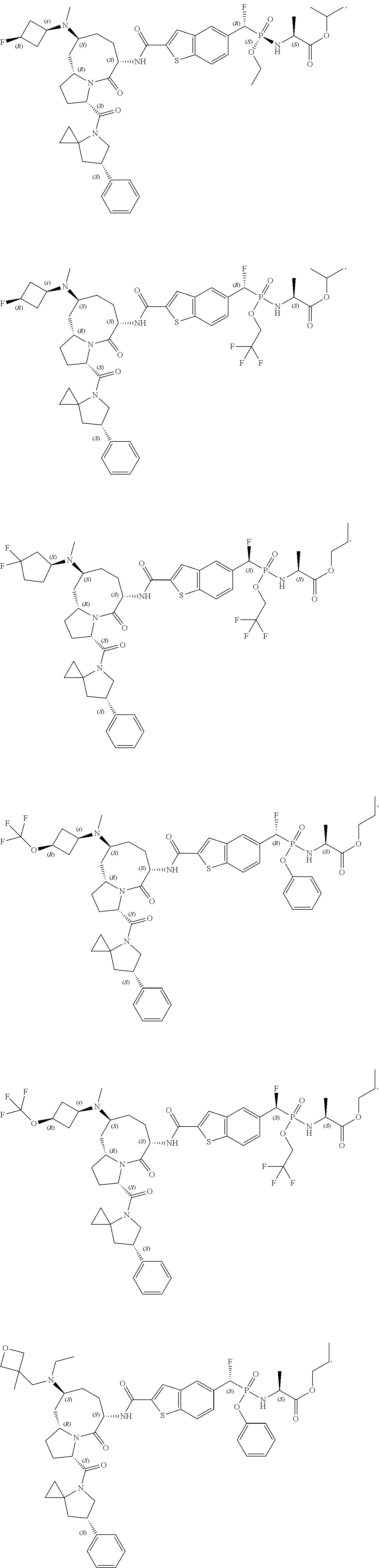 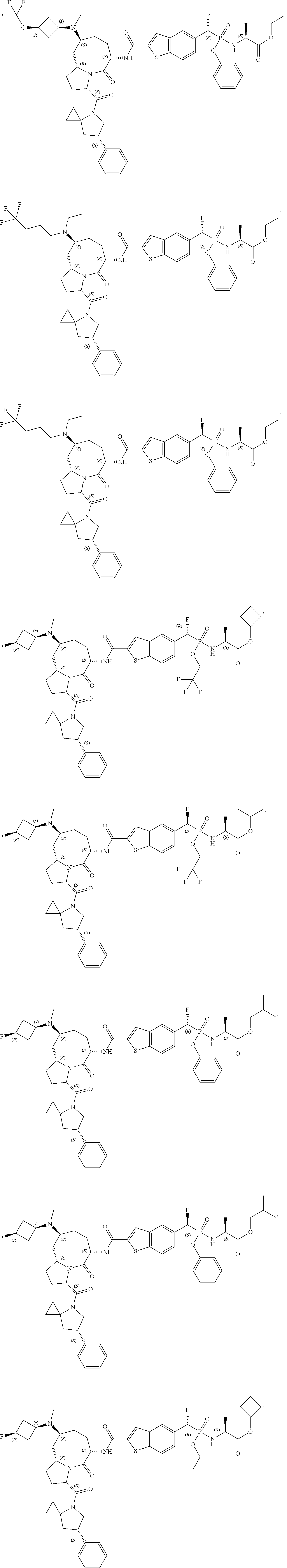 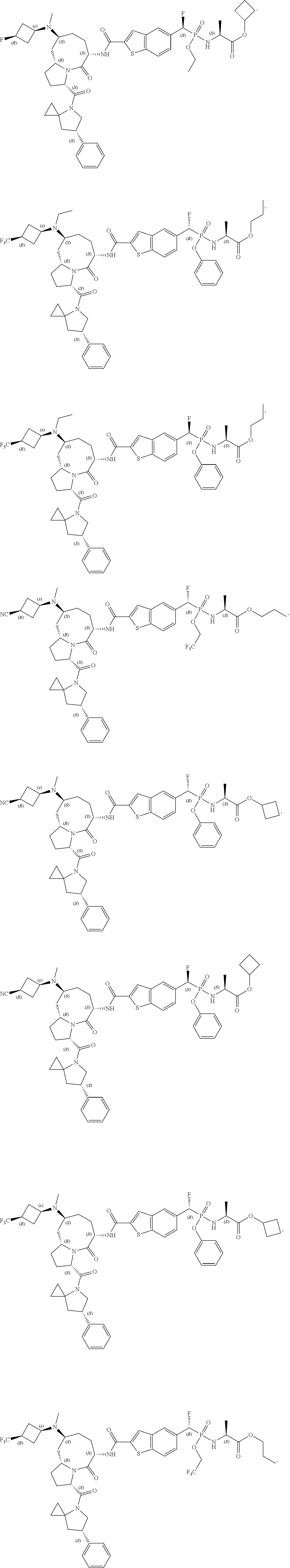 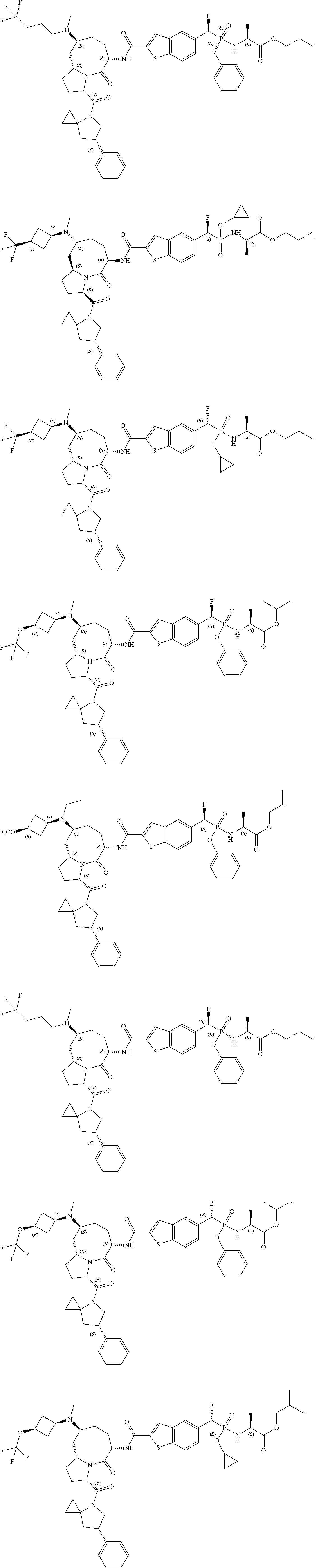 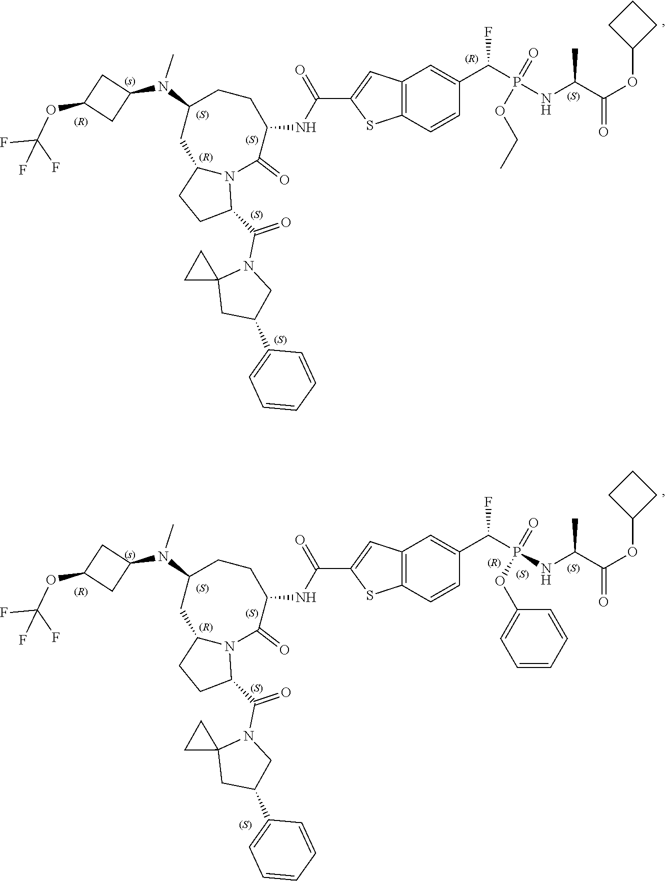 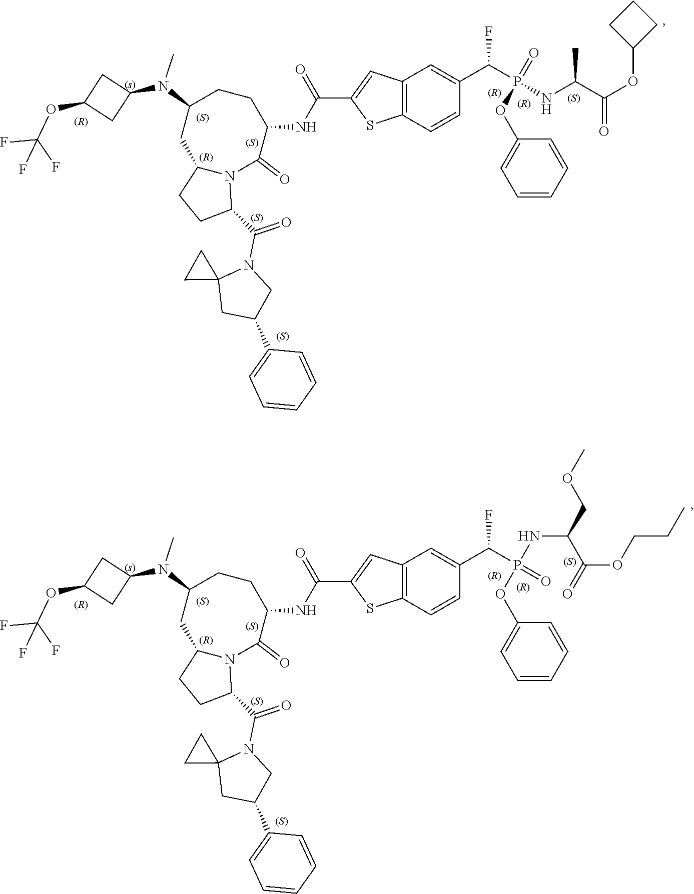 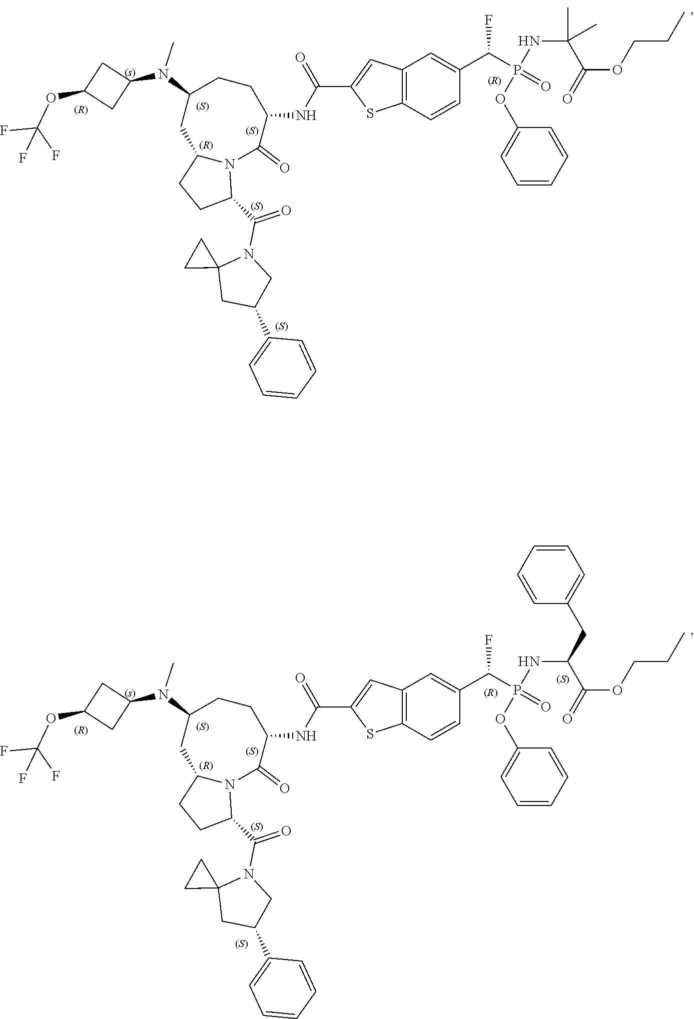 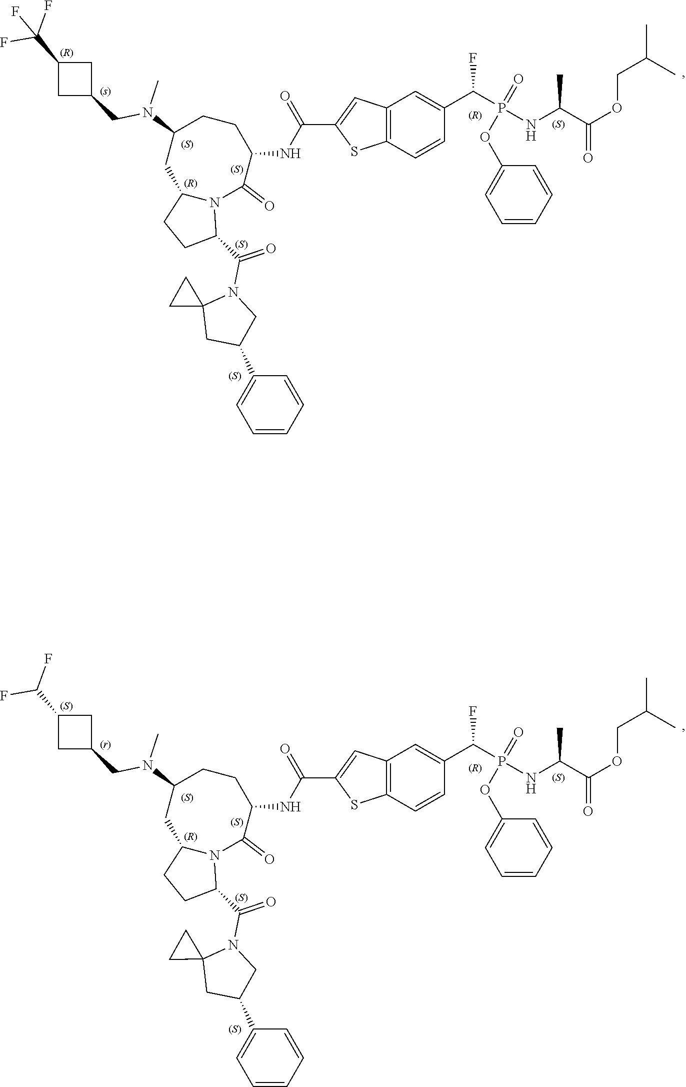 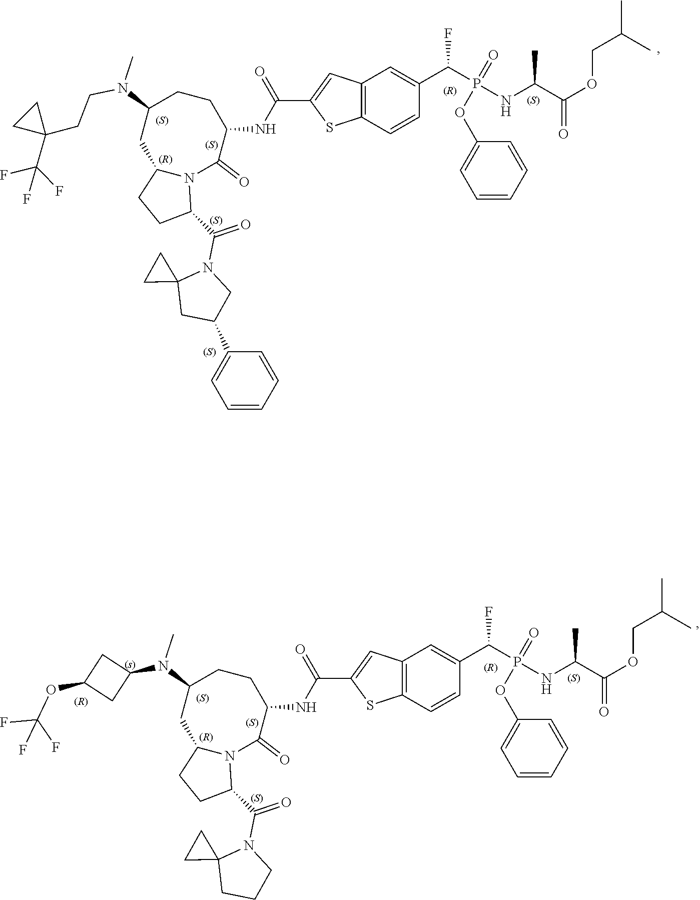 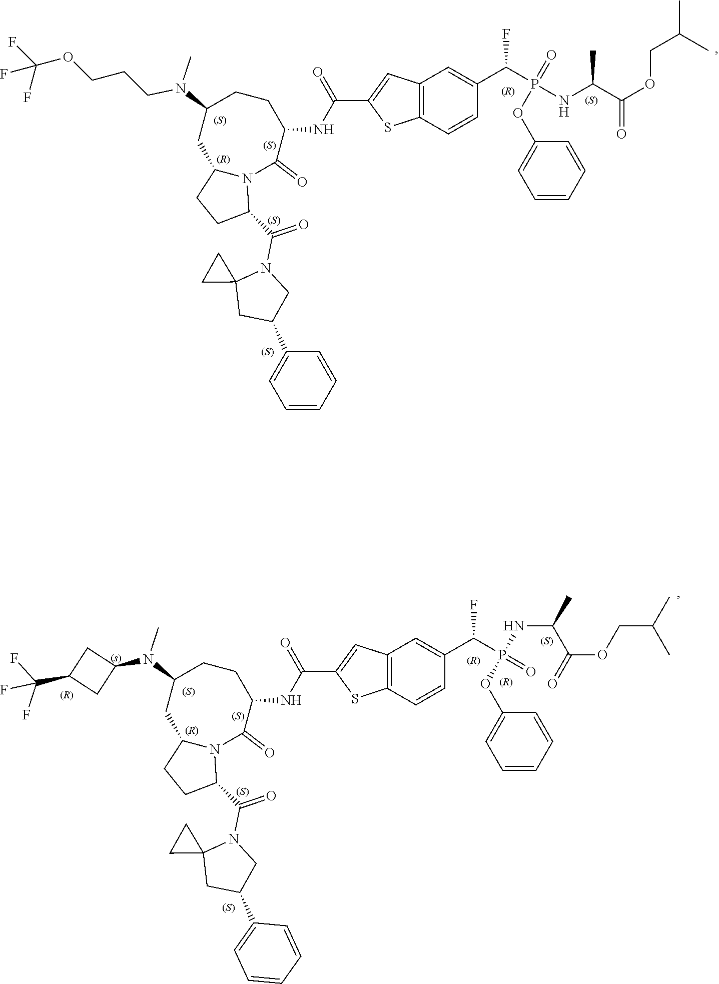 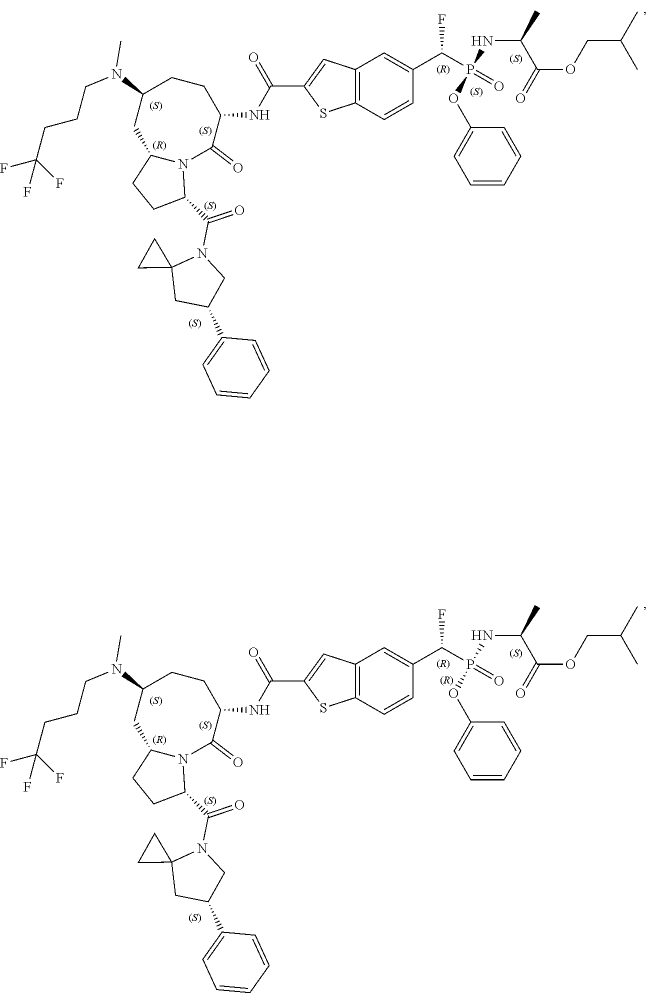 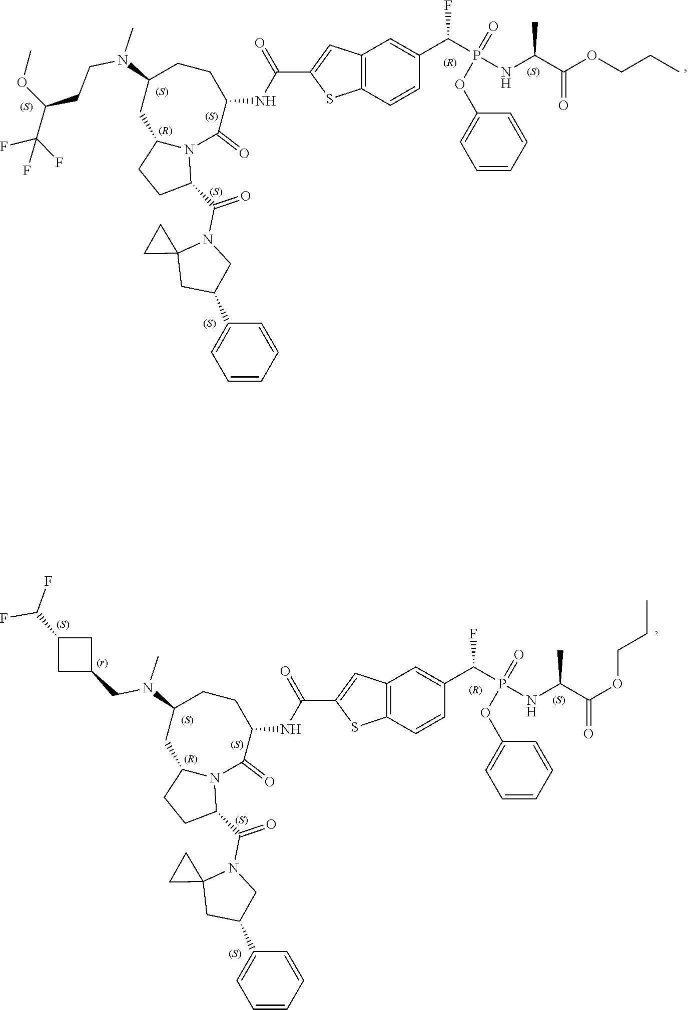 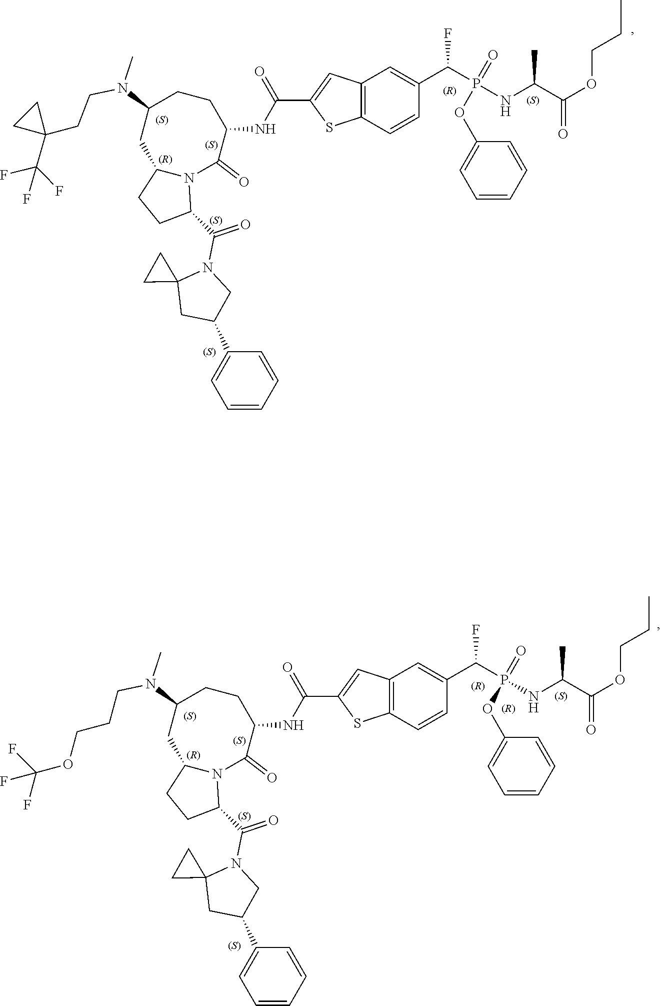 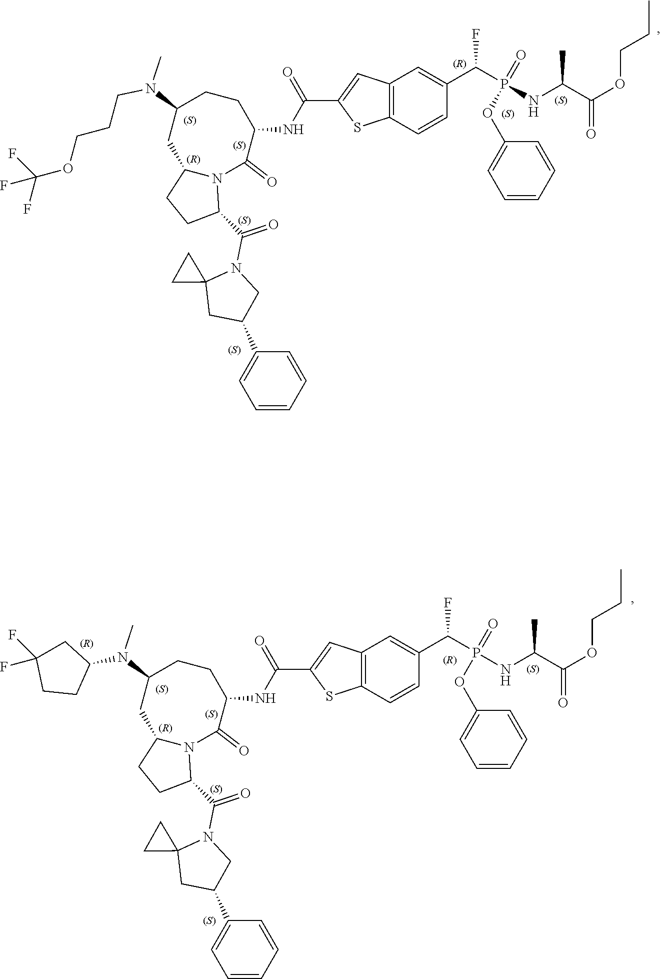 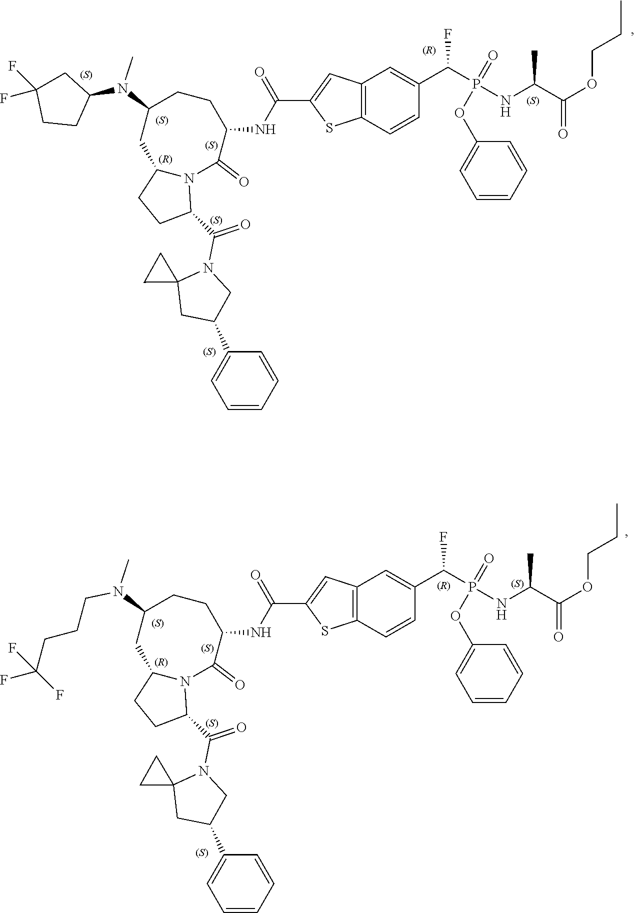 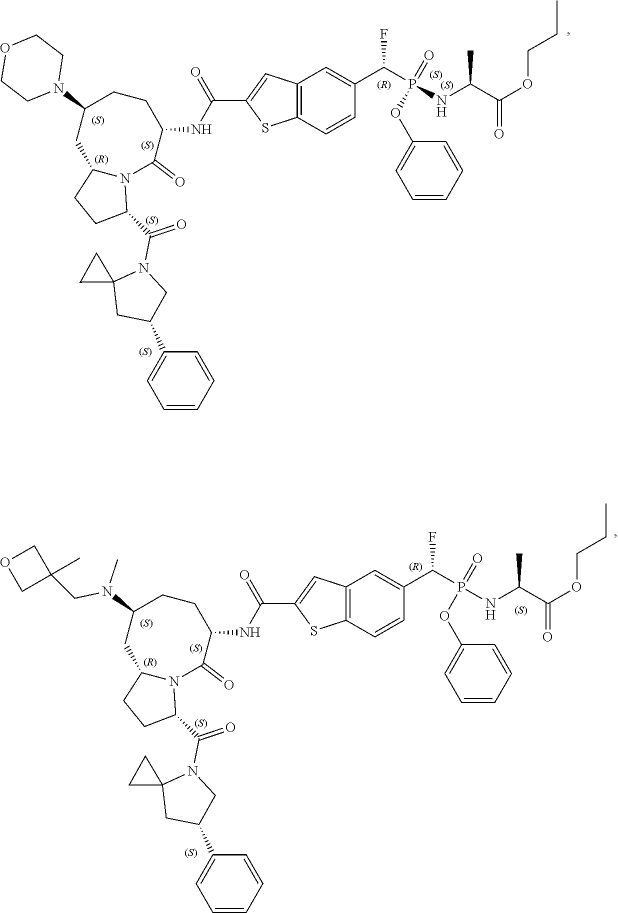 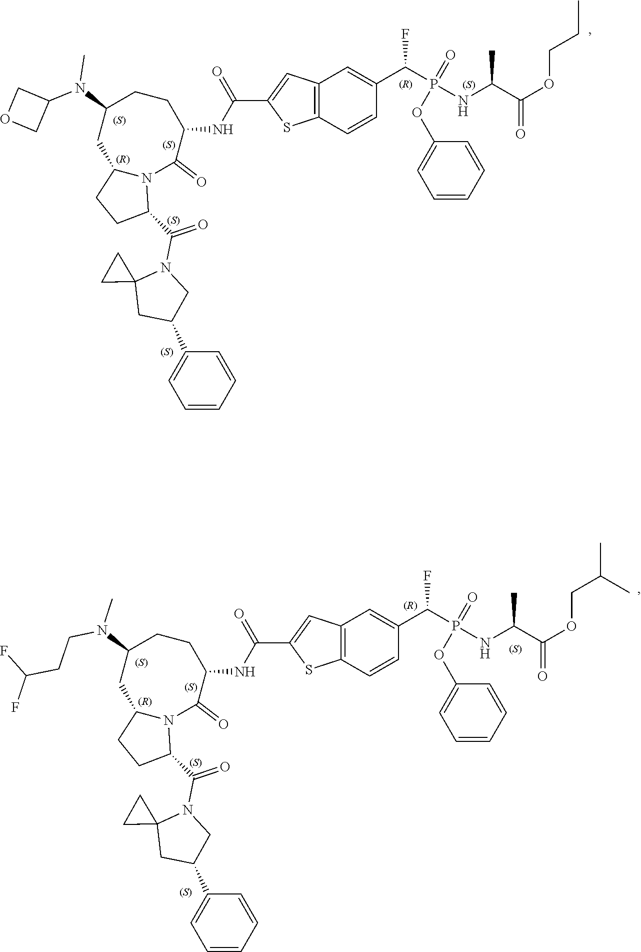 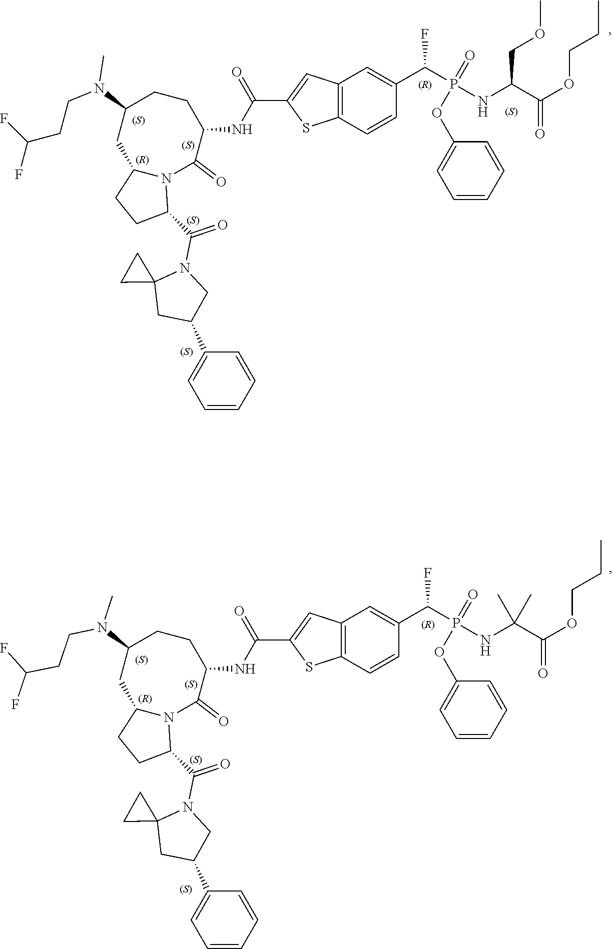 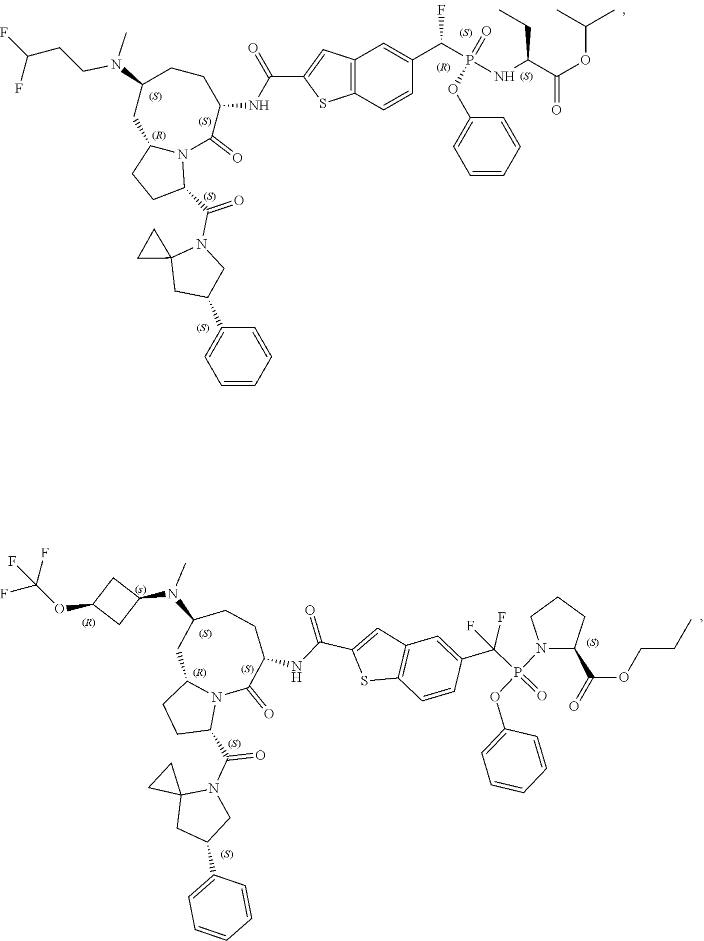 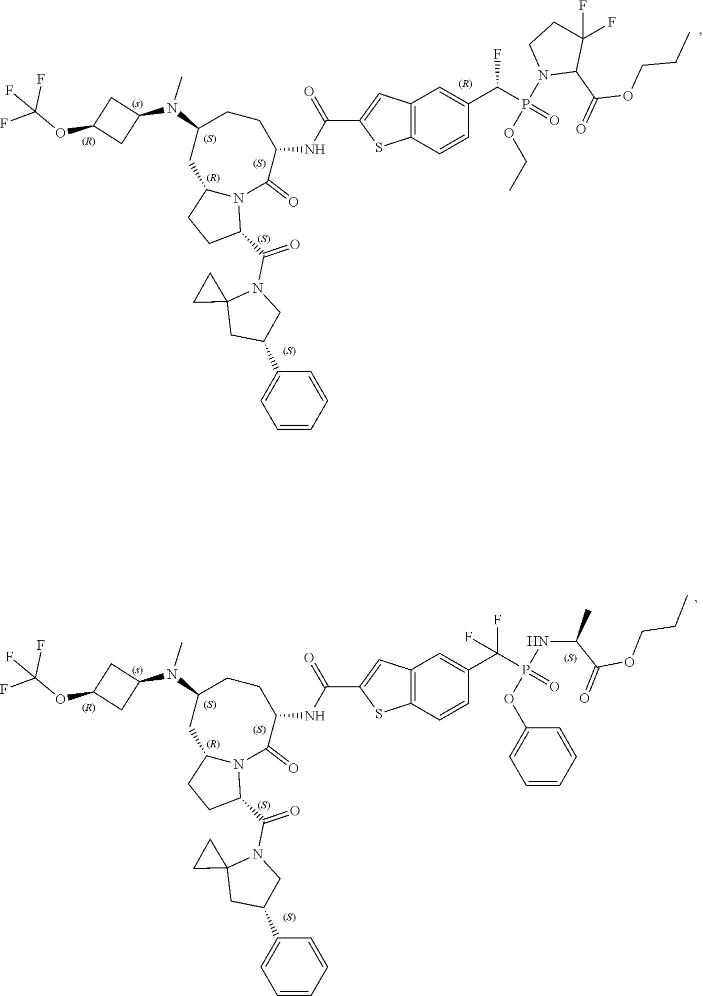 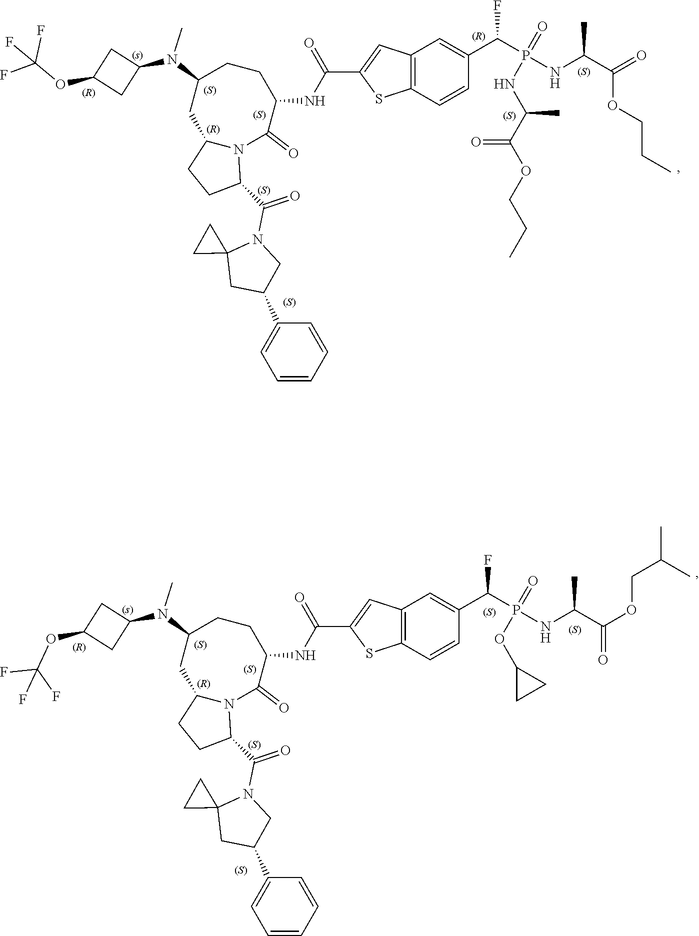 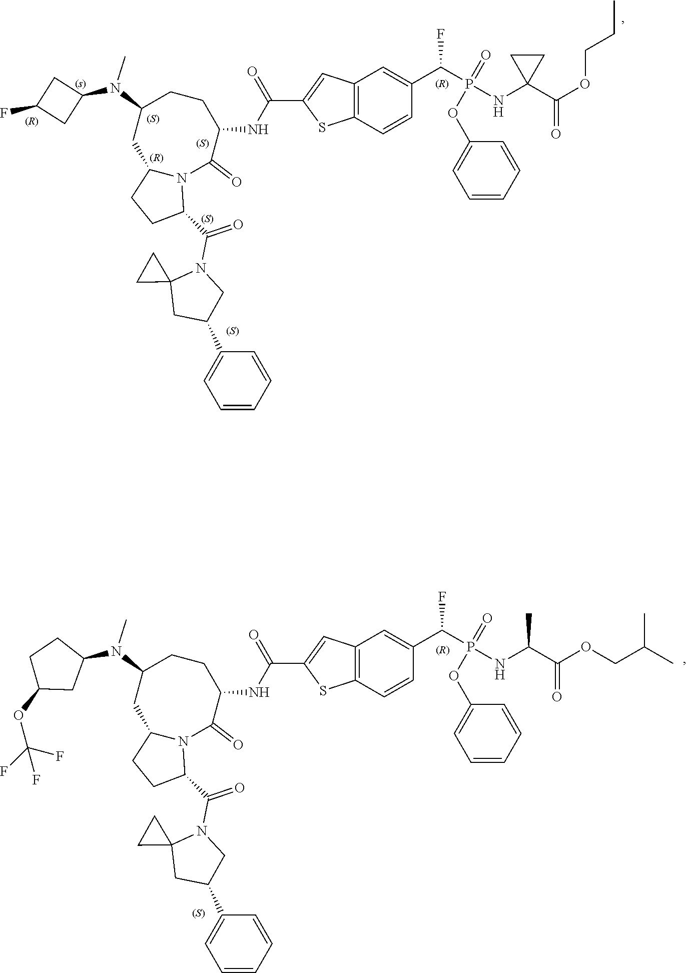 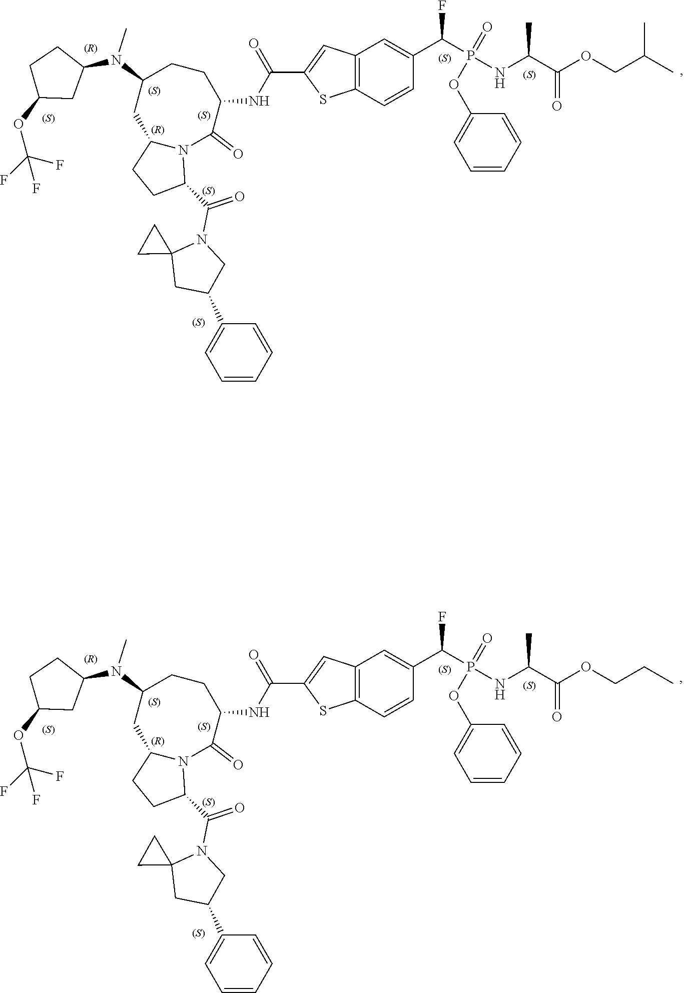 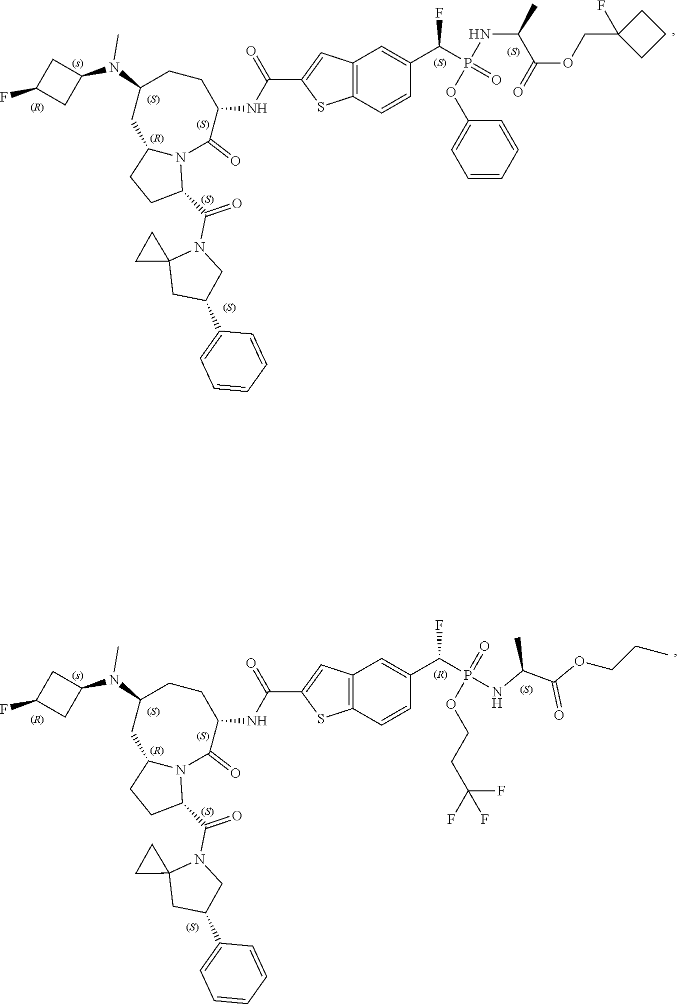 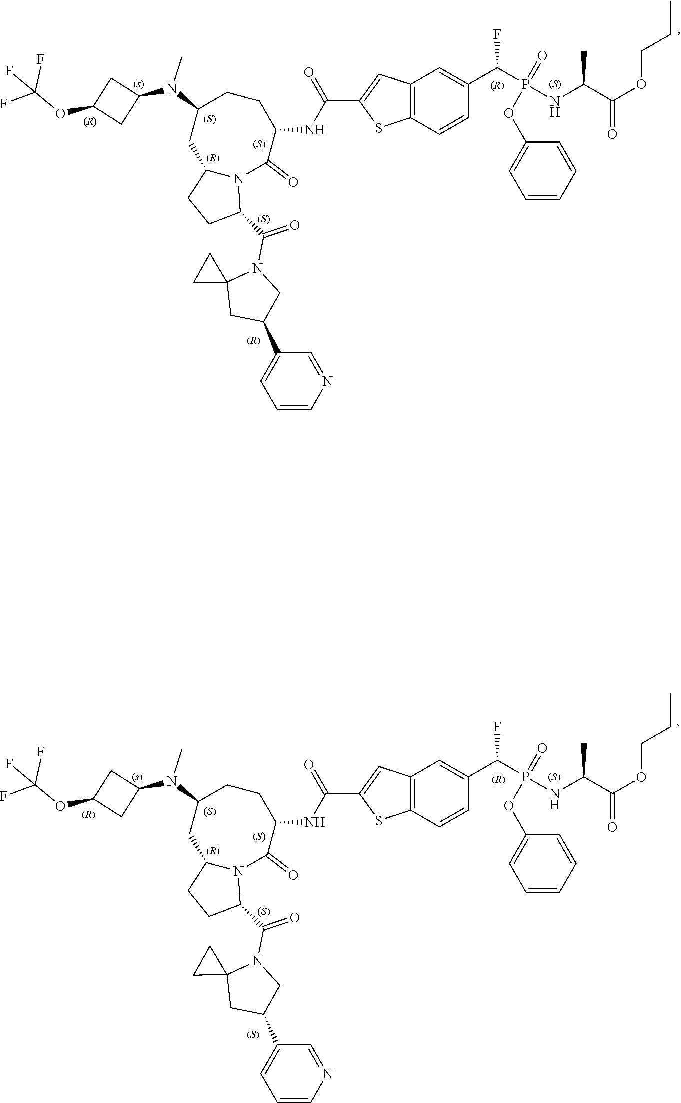 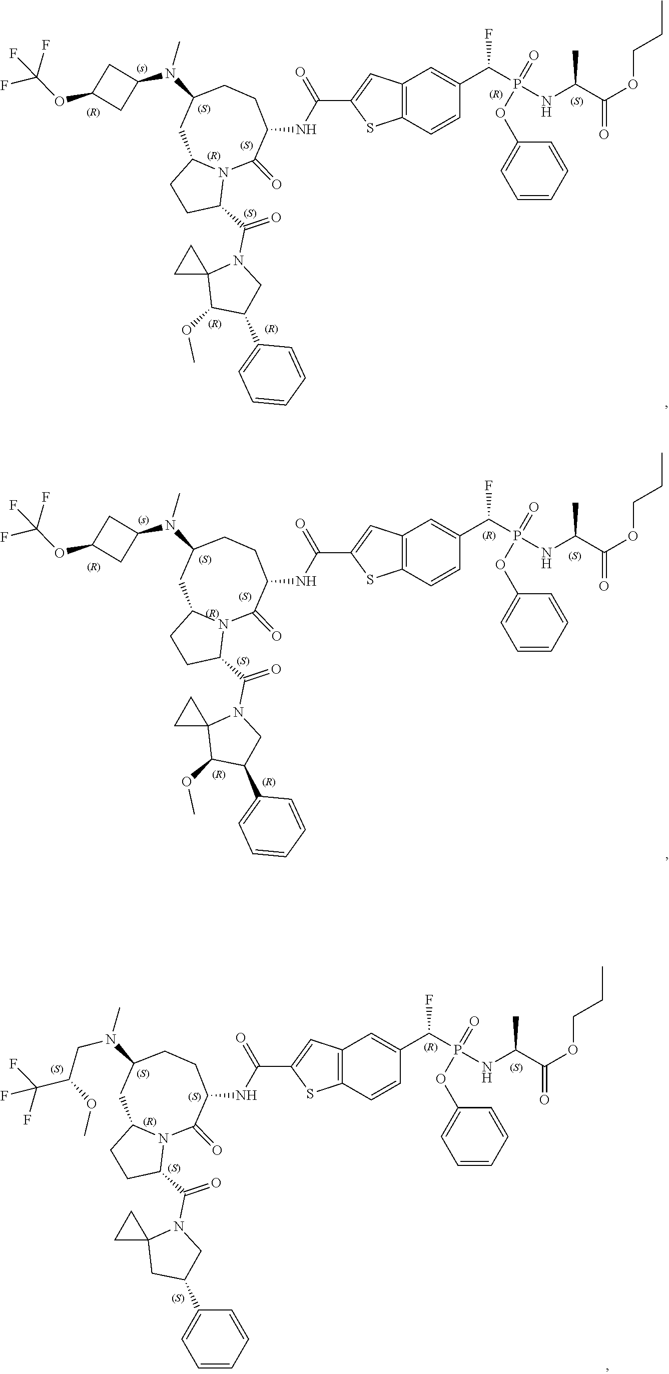 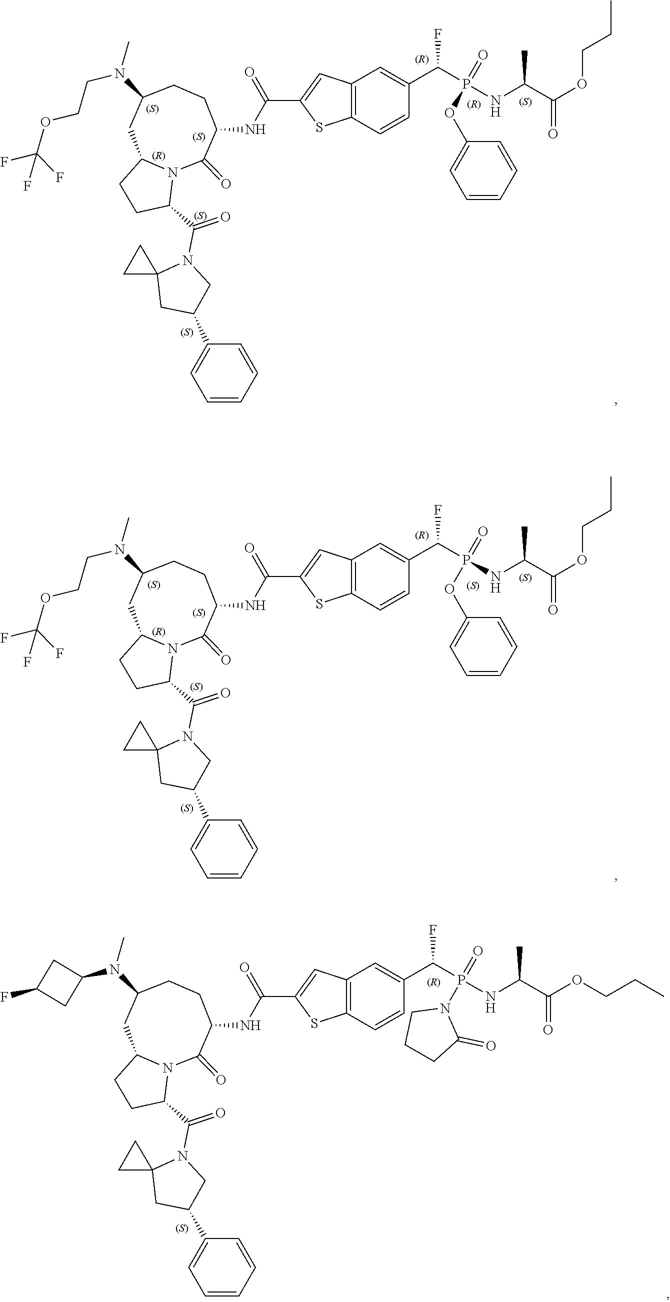 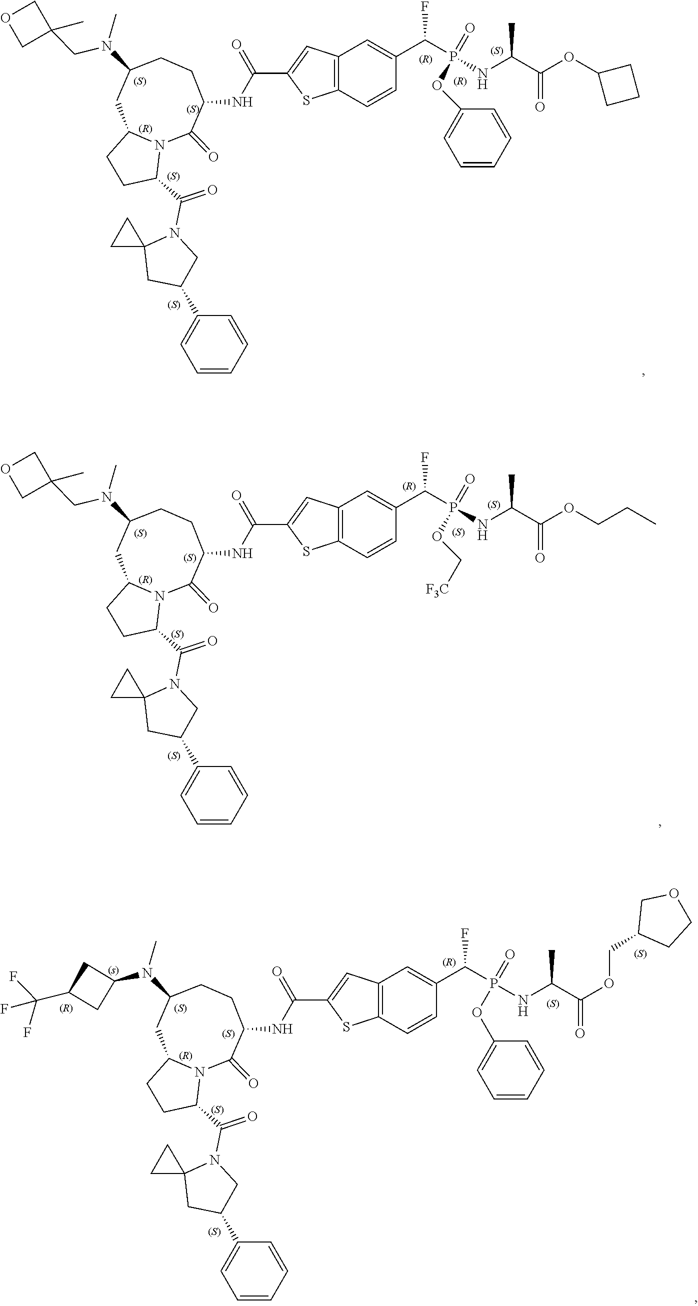 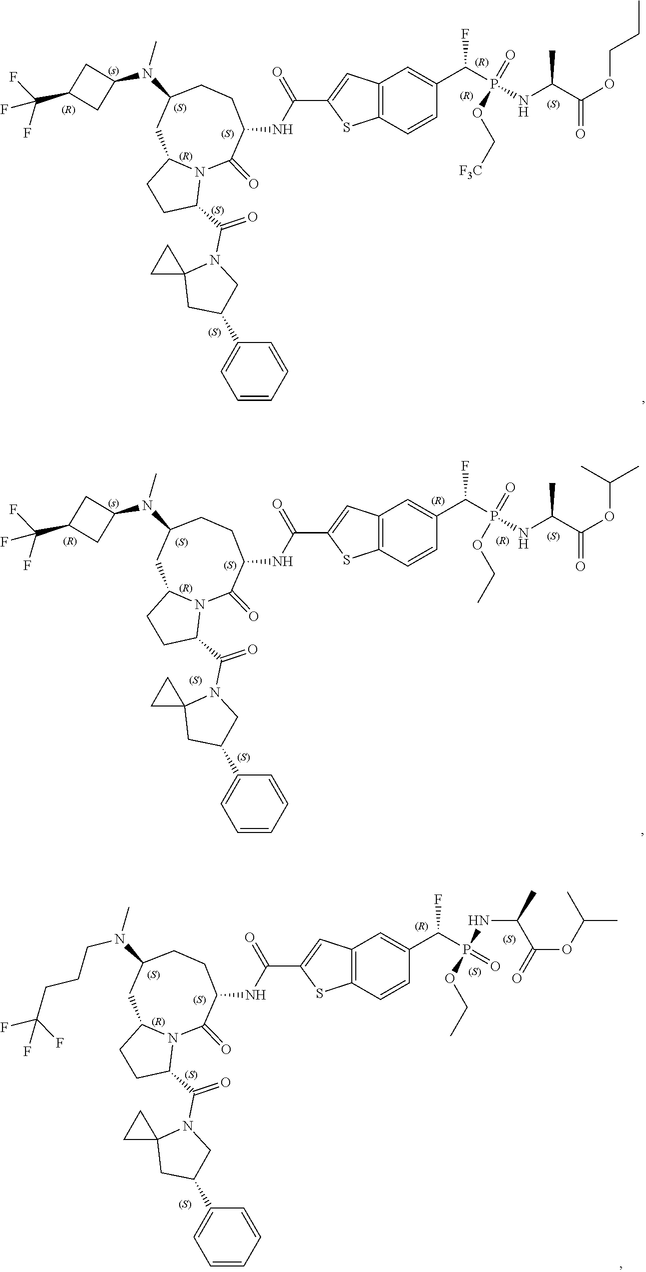 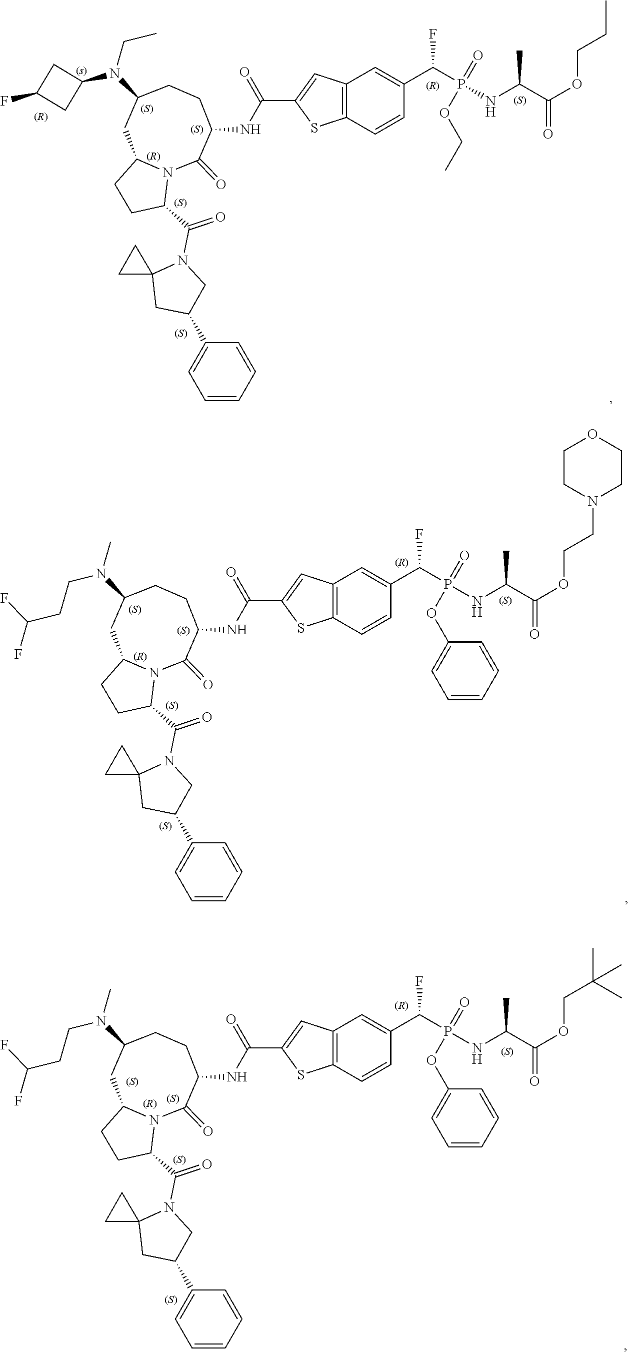 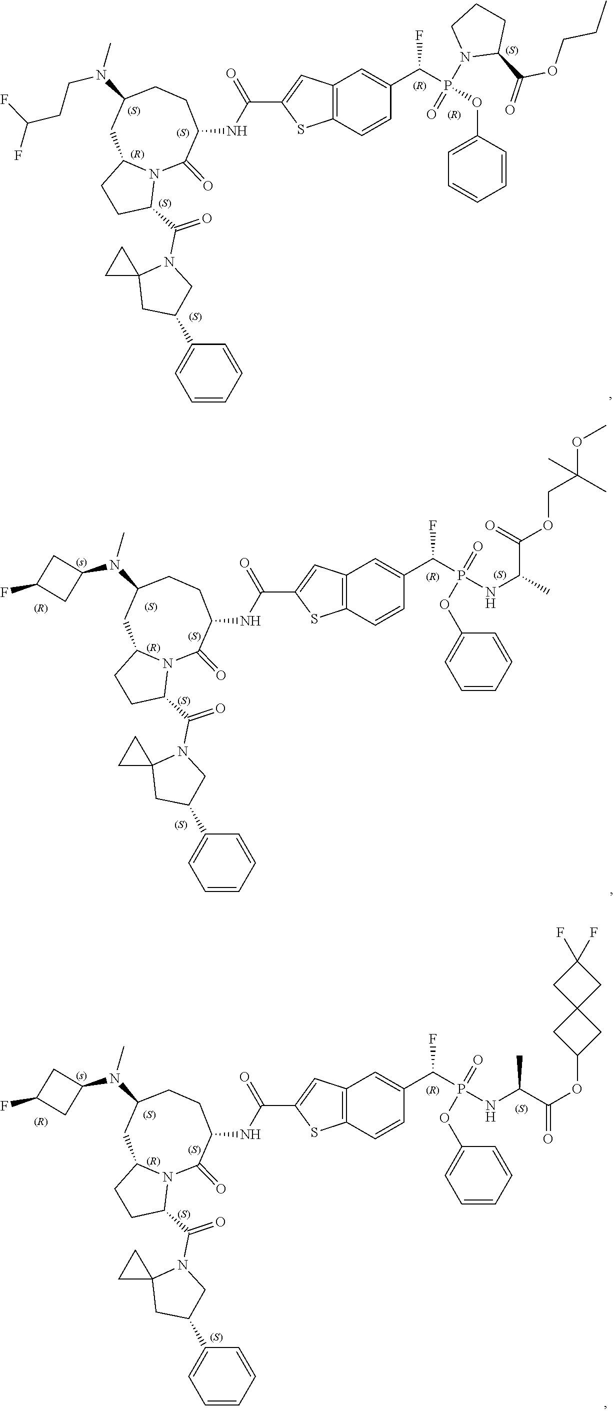 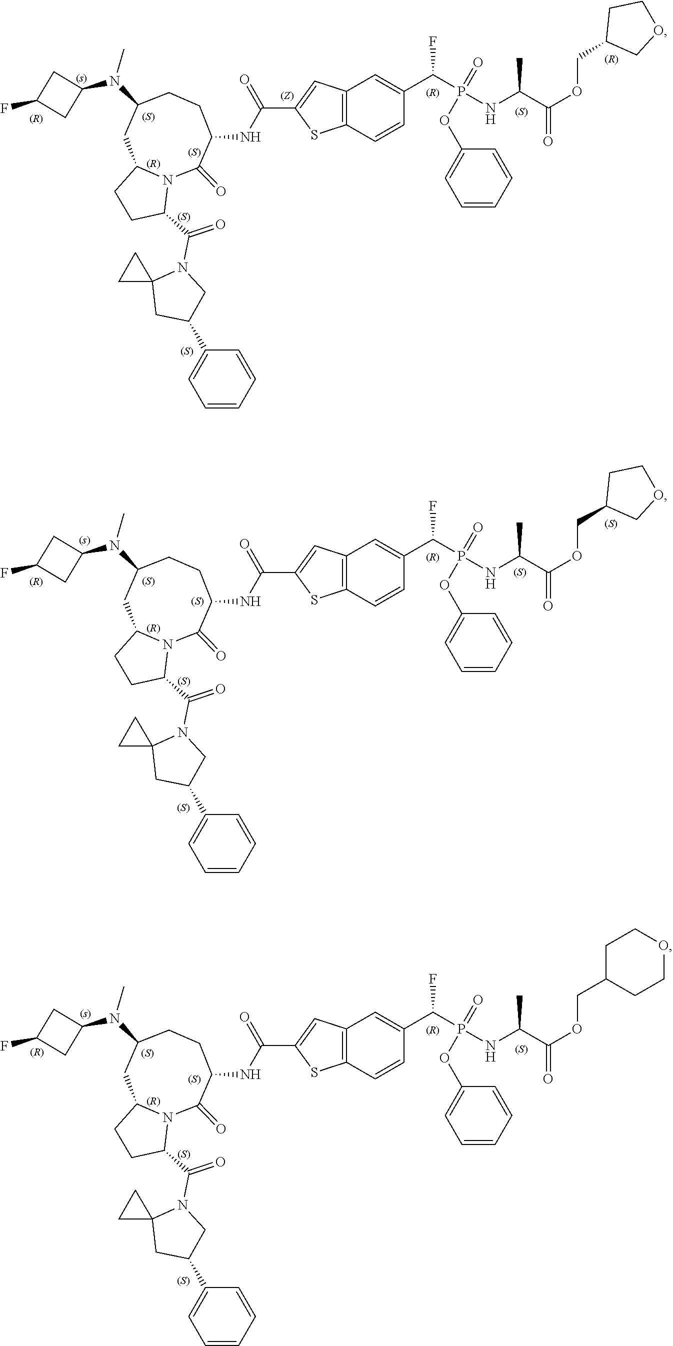 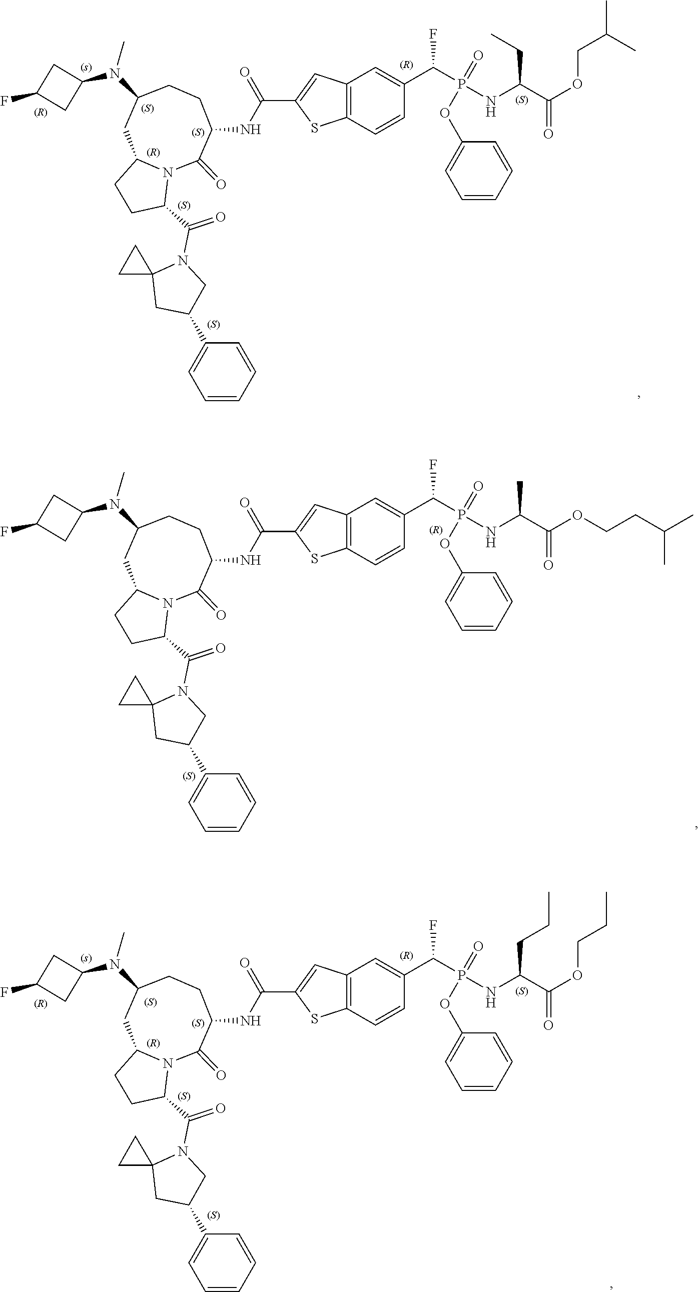 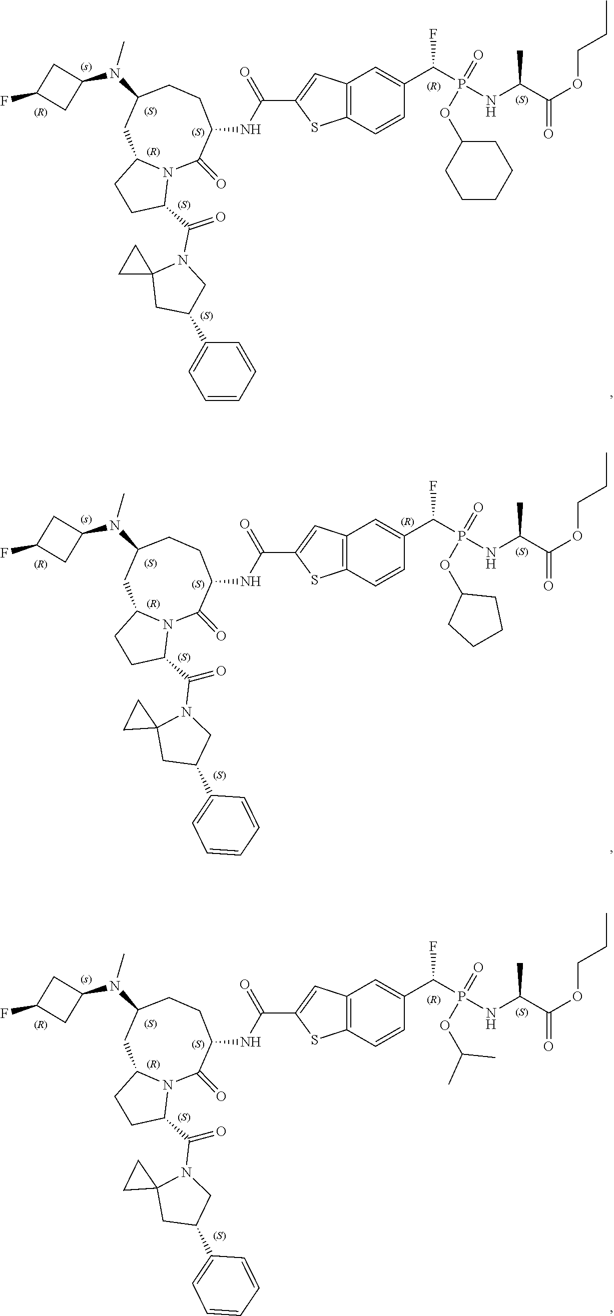 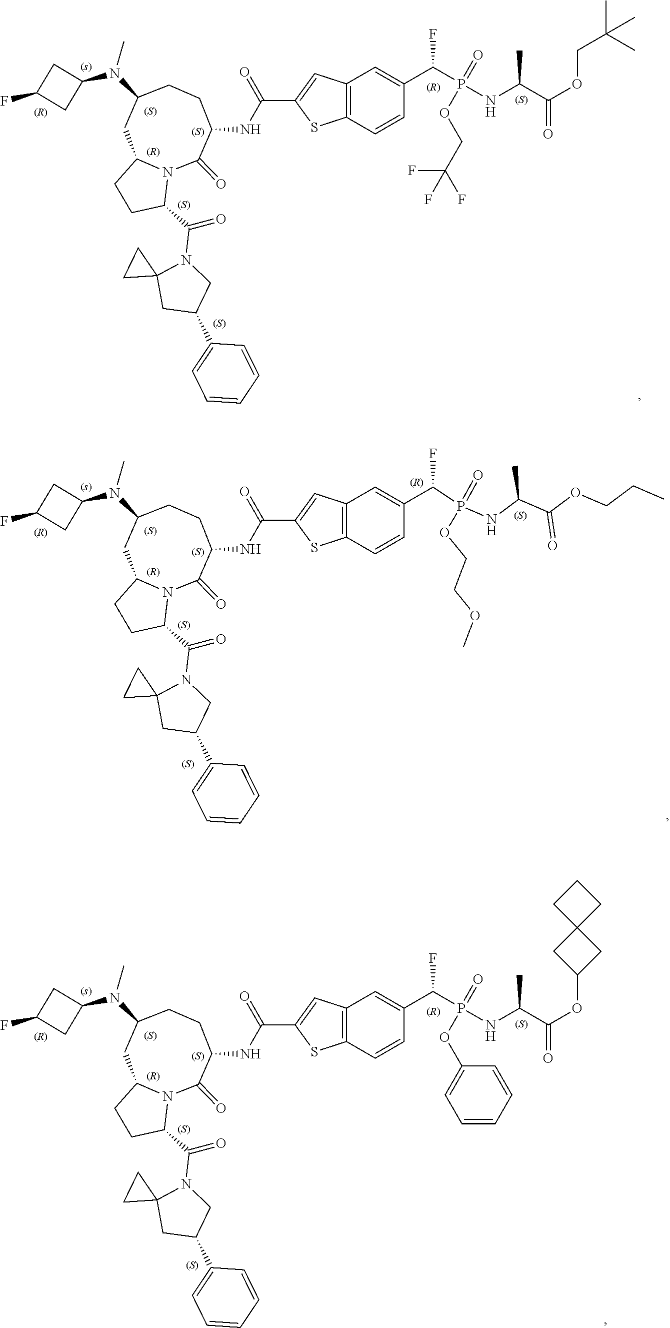 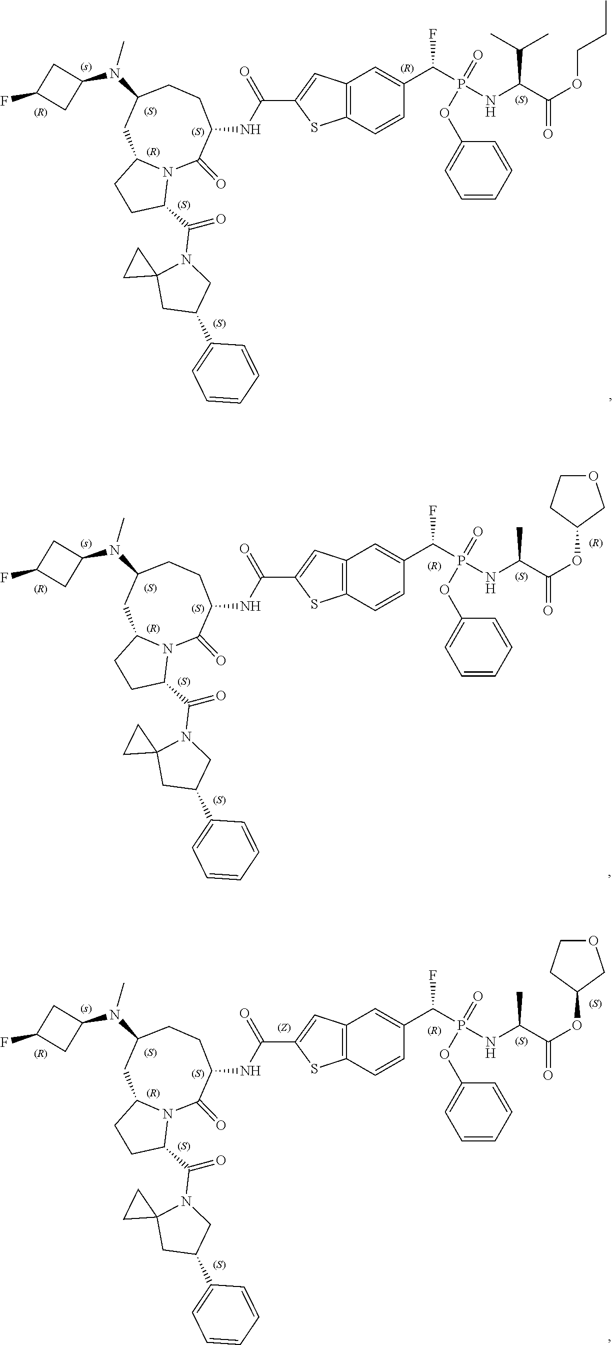 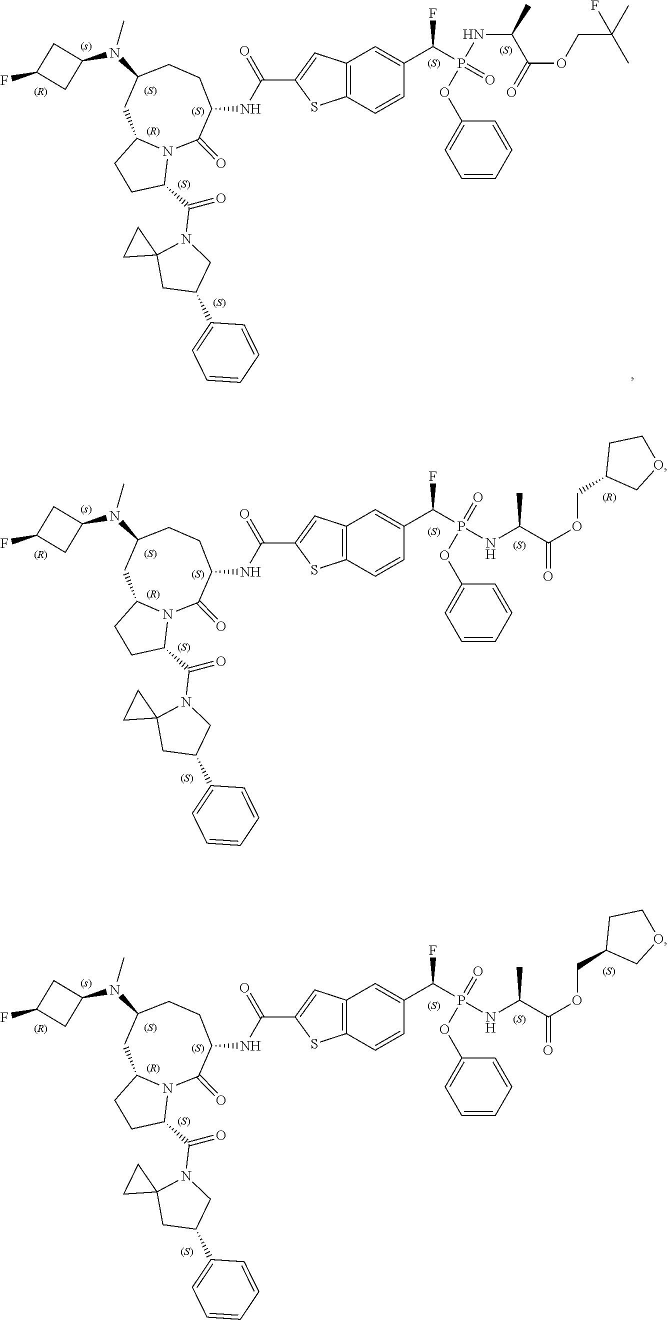 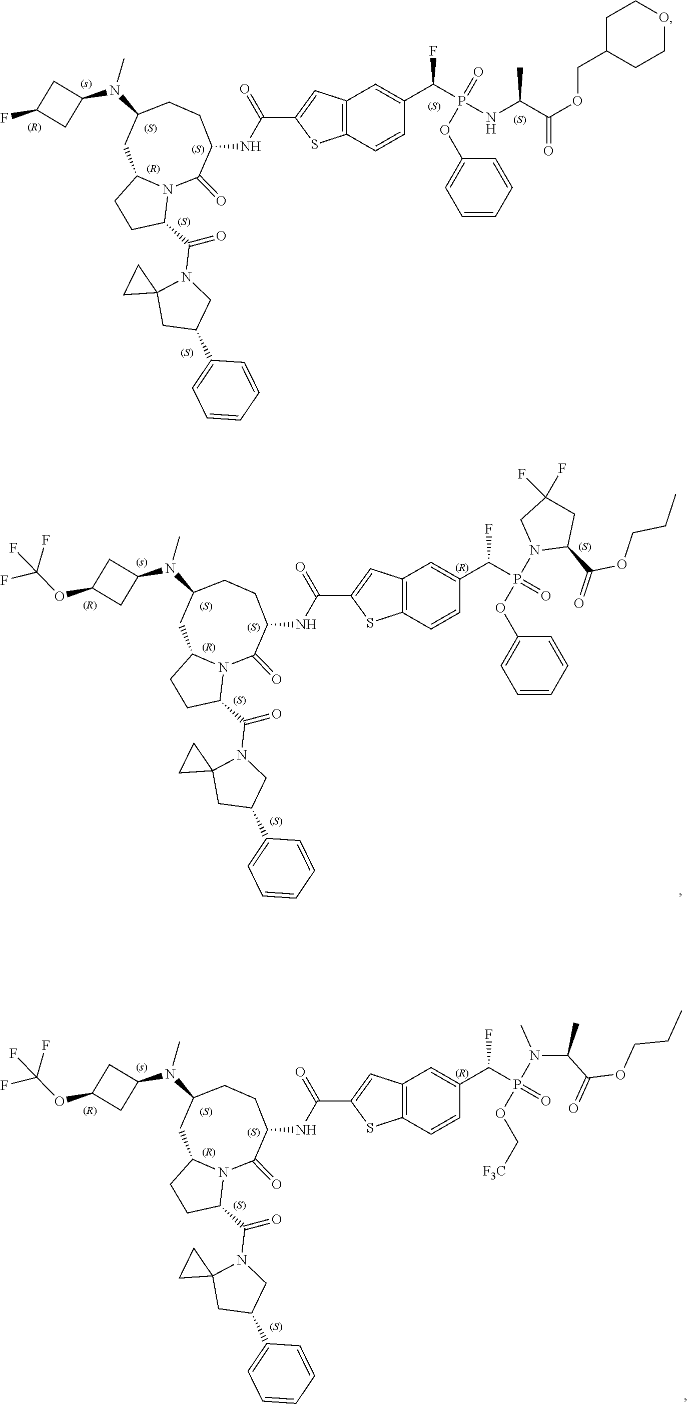 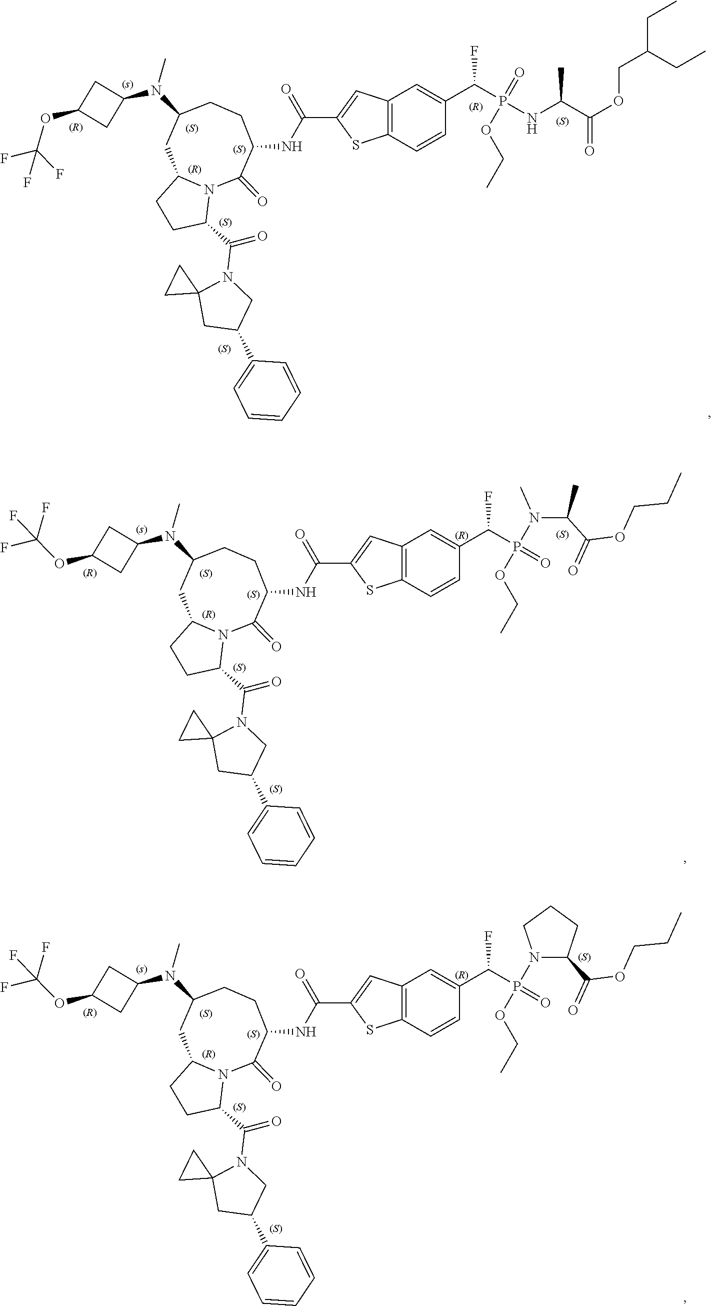 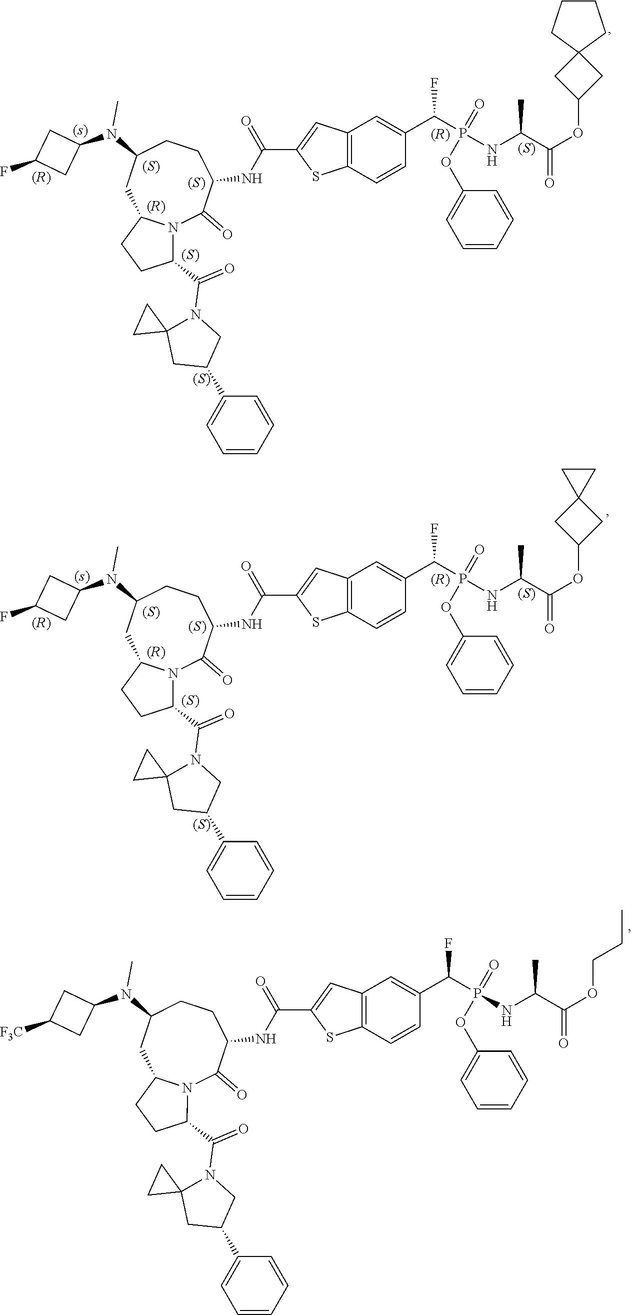 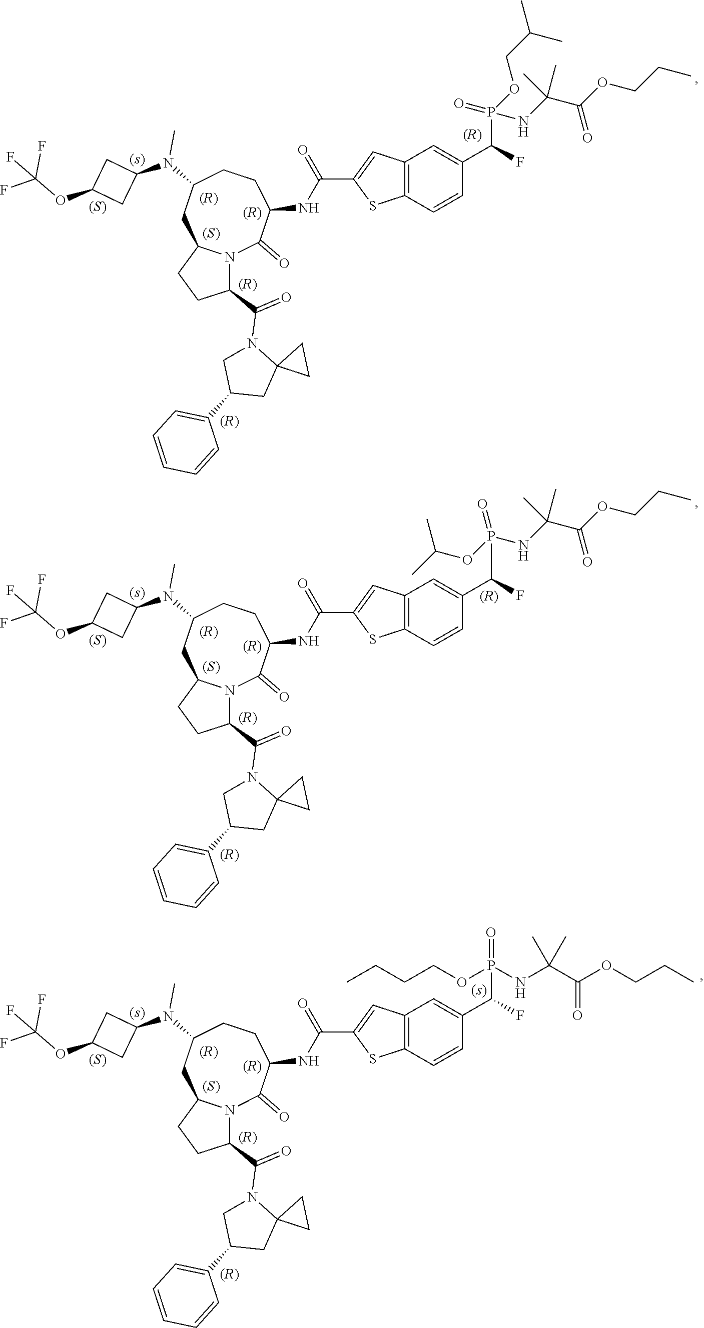 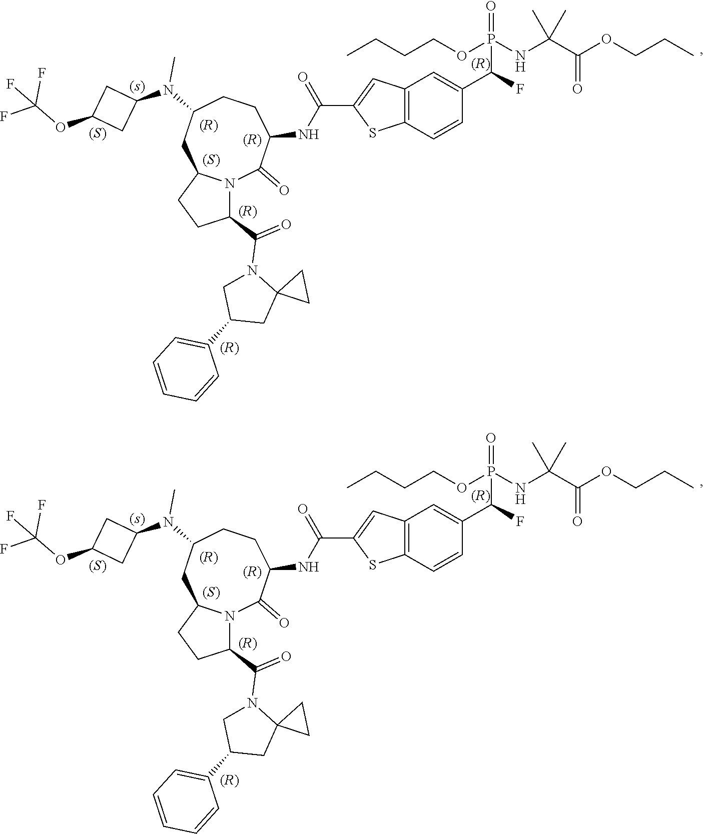
or a pharmaceutically acceptable salt thereof.

4. The compound of claim 1, or a pharmaceutically acceptable salt thereof, wherein R^{P1} and R^{P2} are each —OH.

5. The compound of claim 1, wherein the compound is selected from the group consisting of: 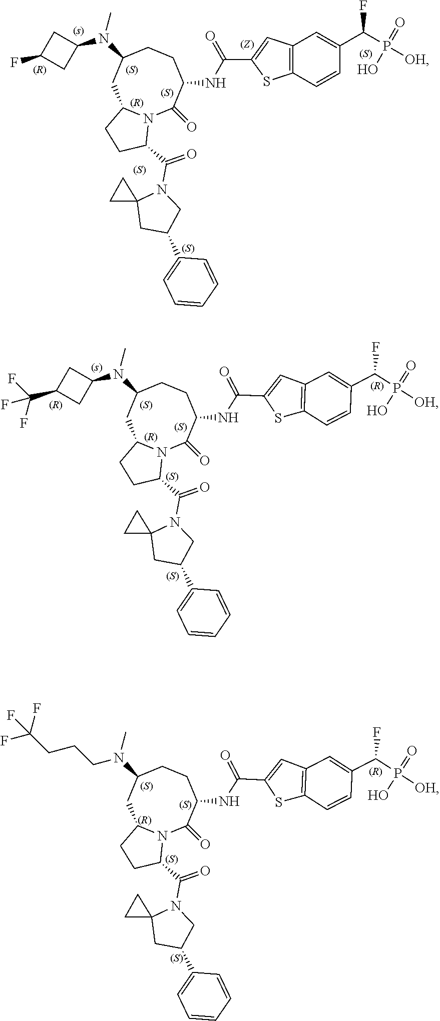 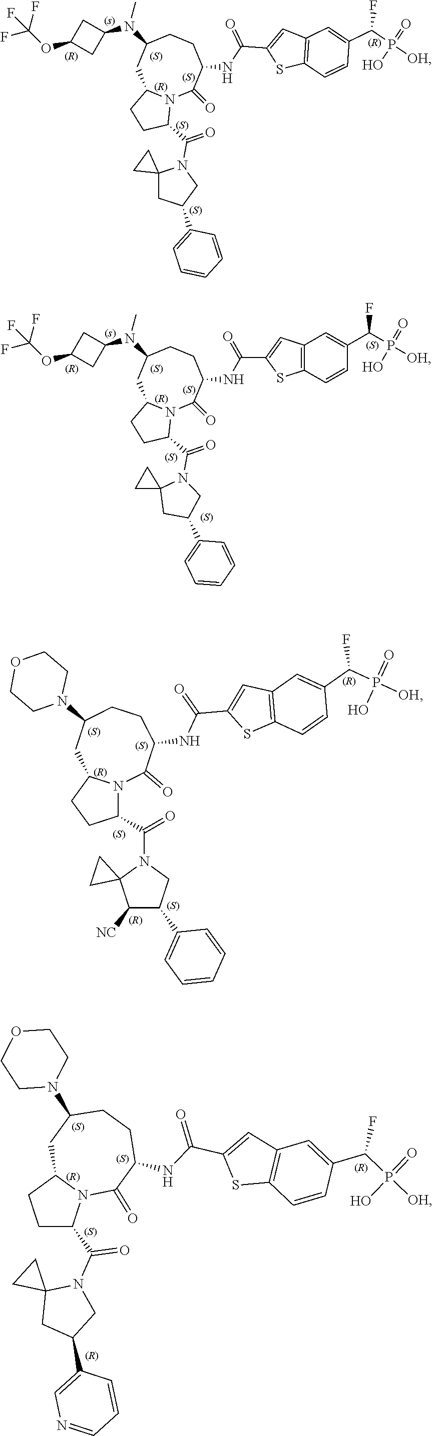 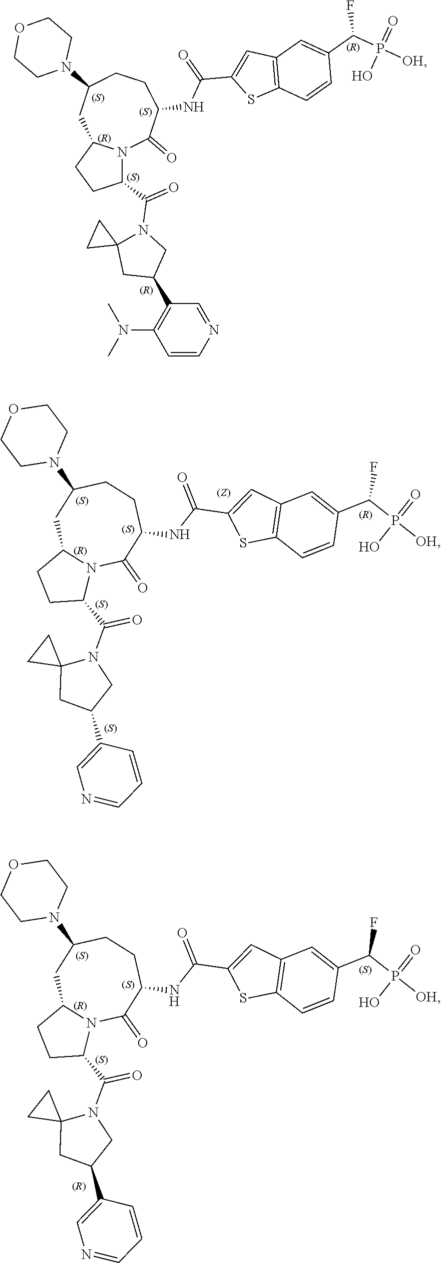 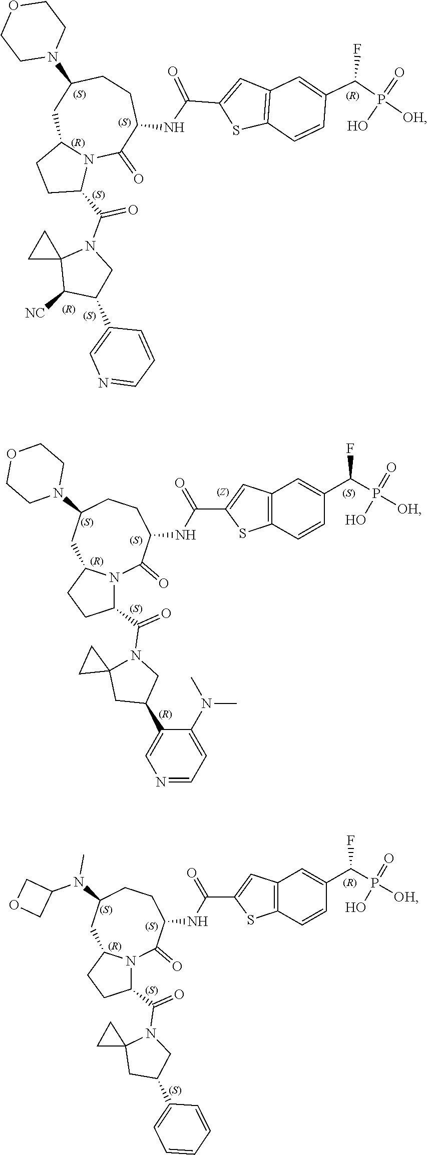 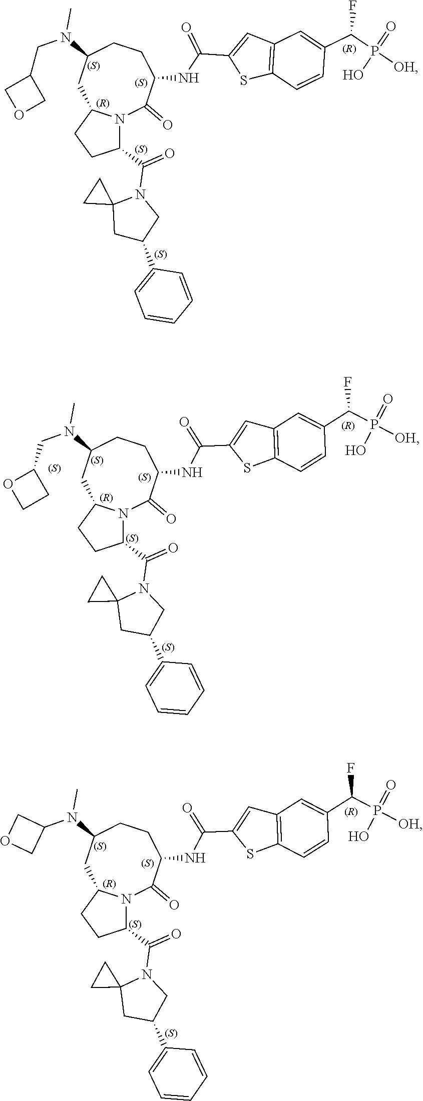 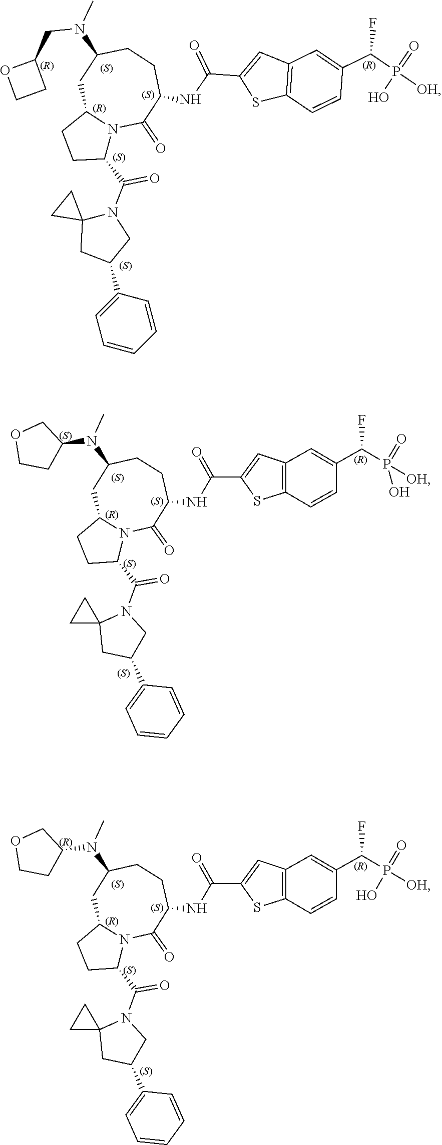 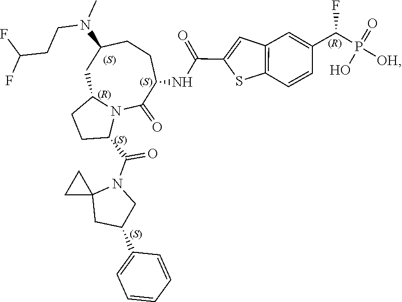 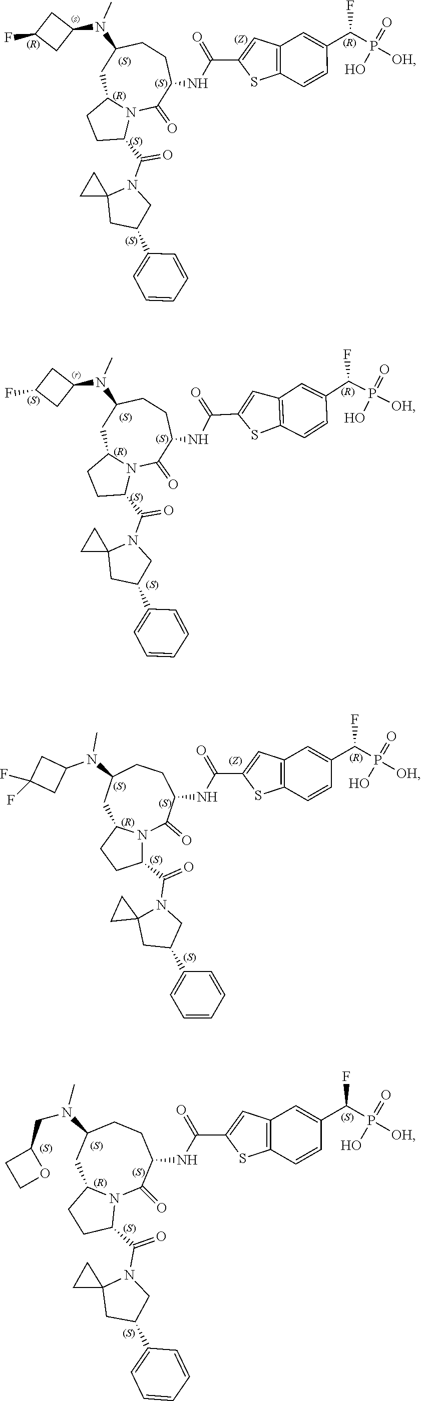 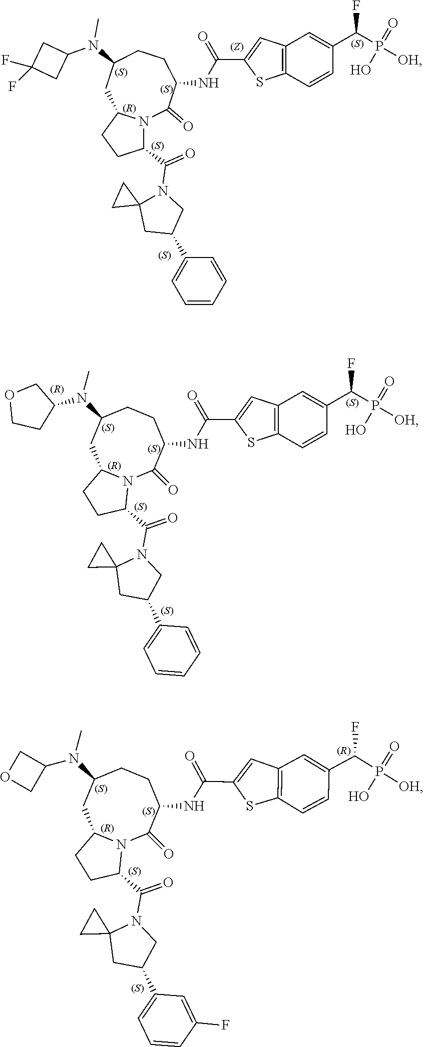 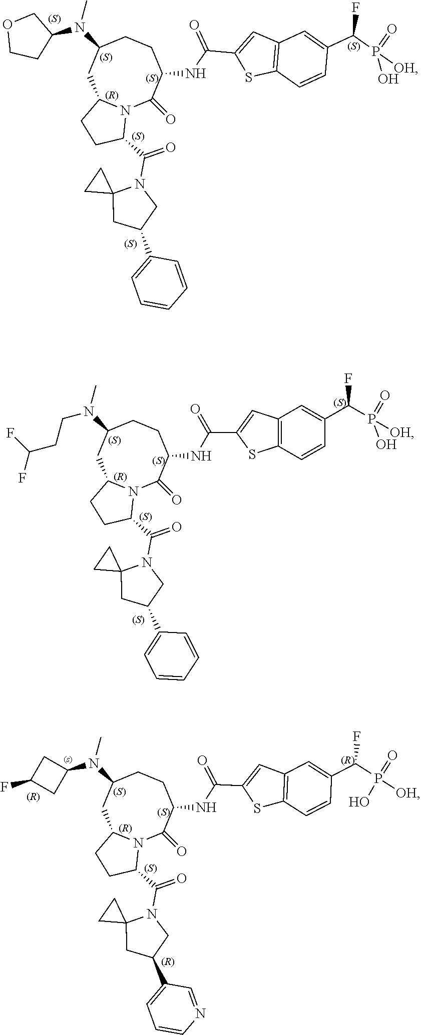 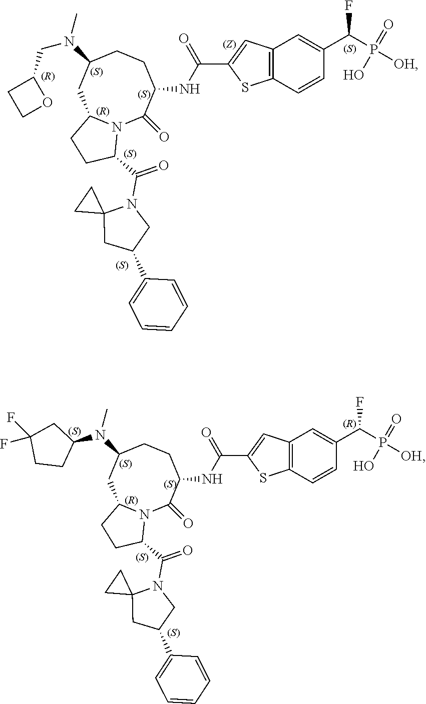 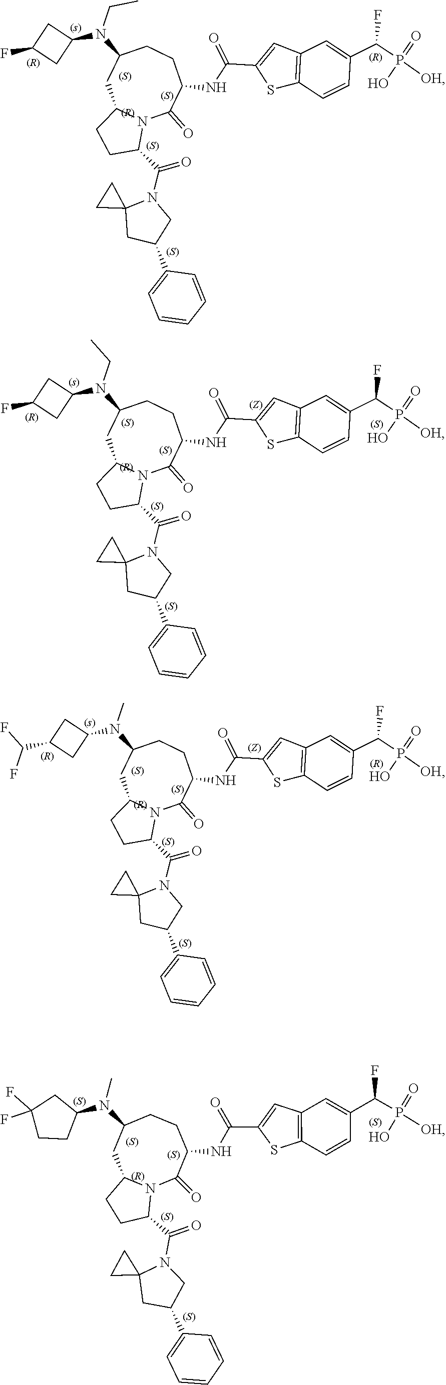 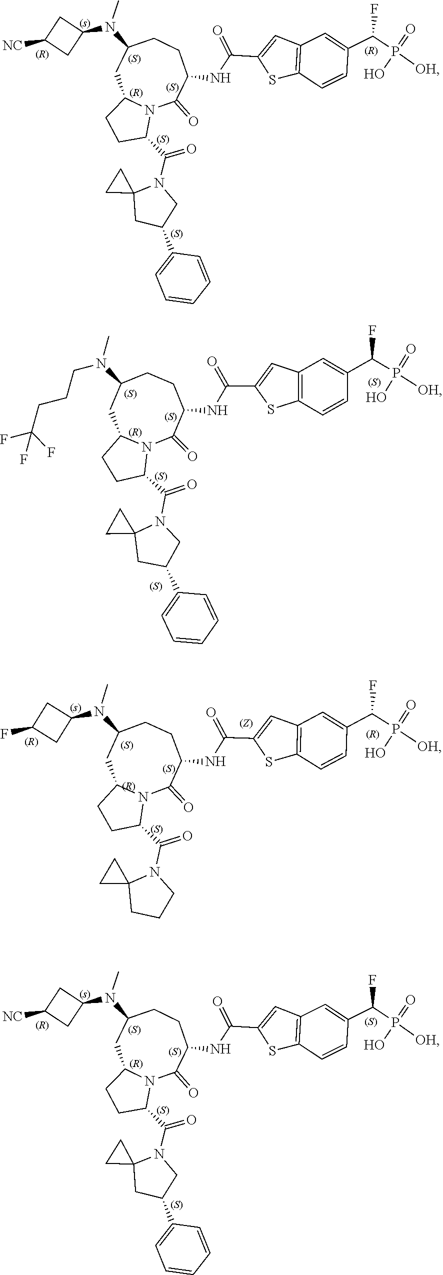 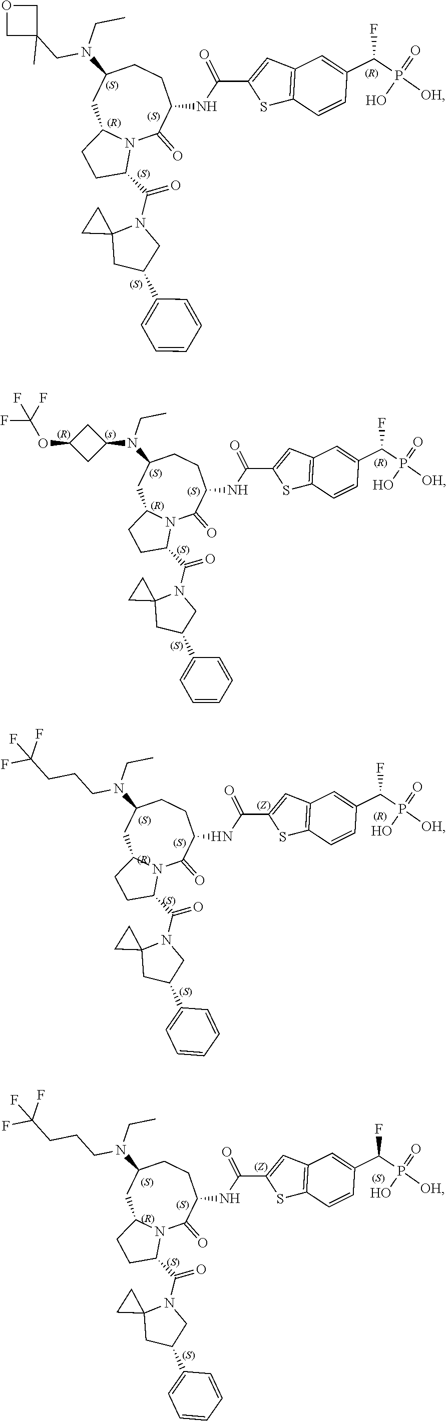 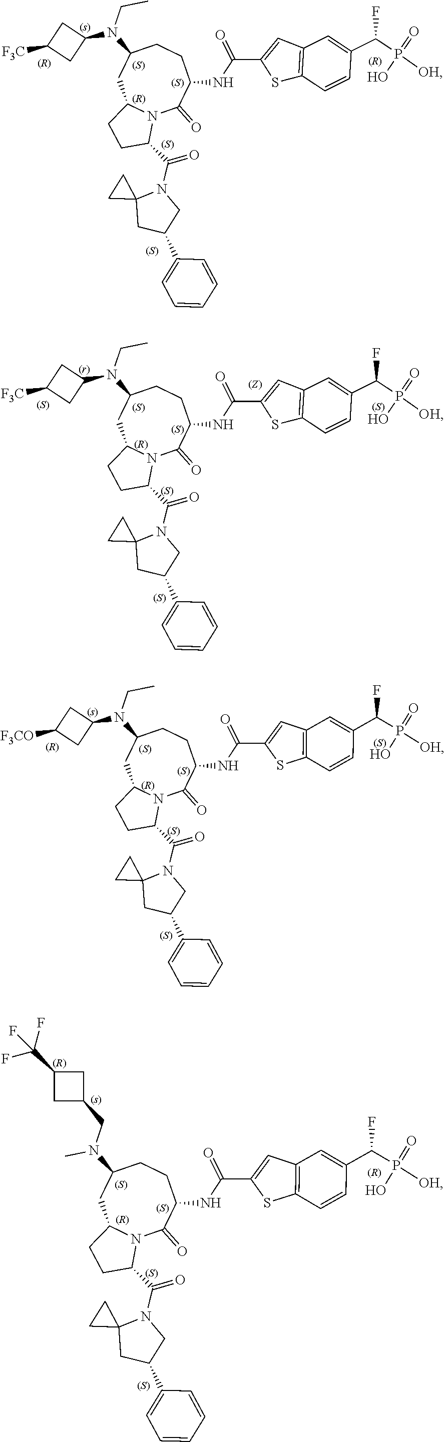 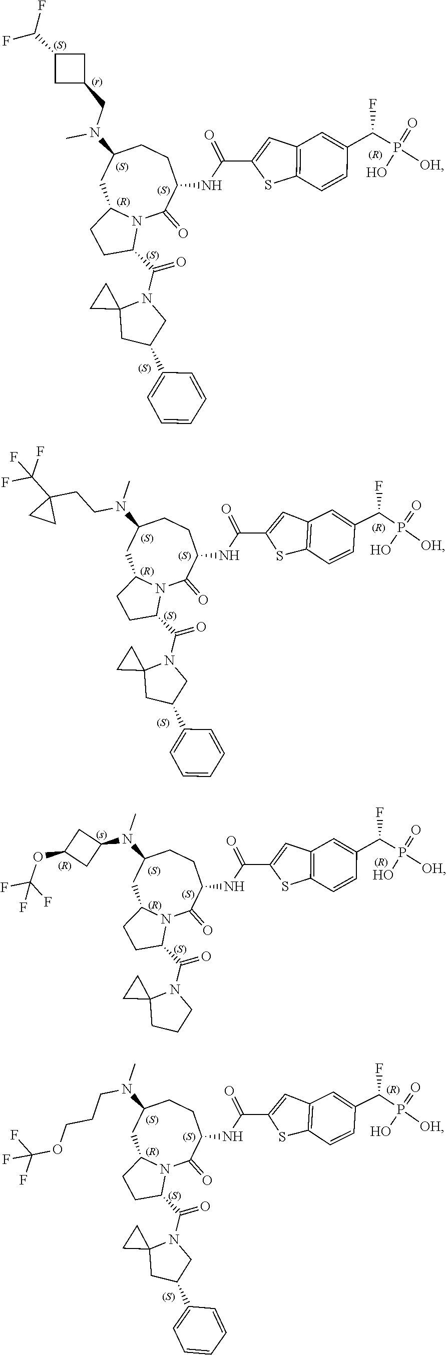 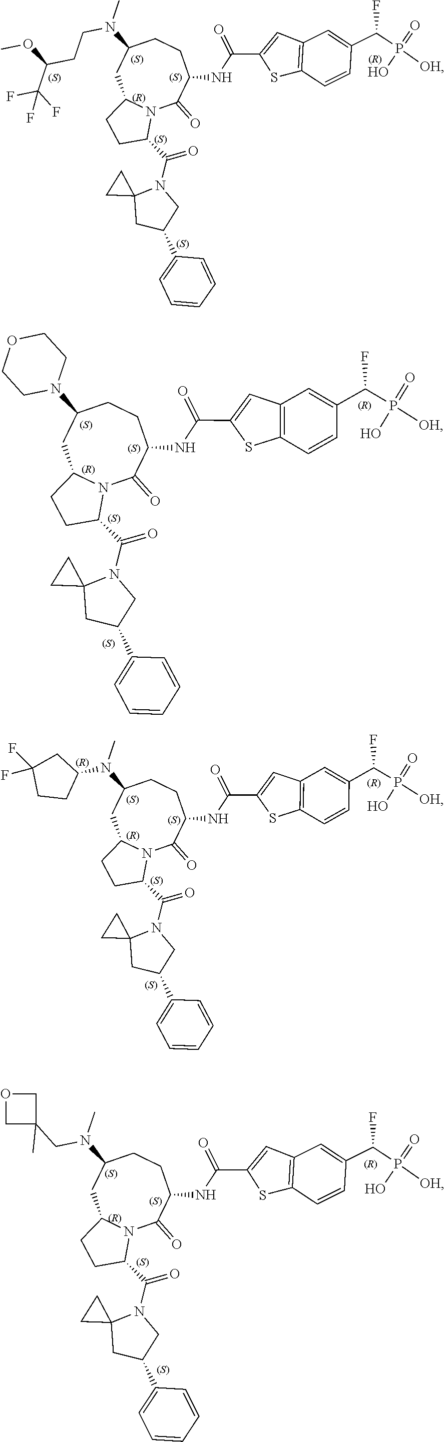 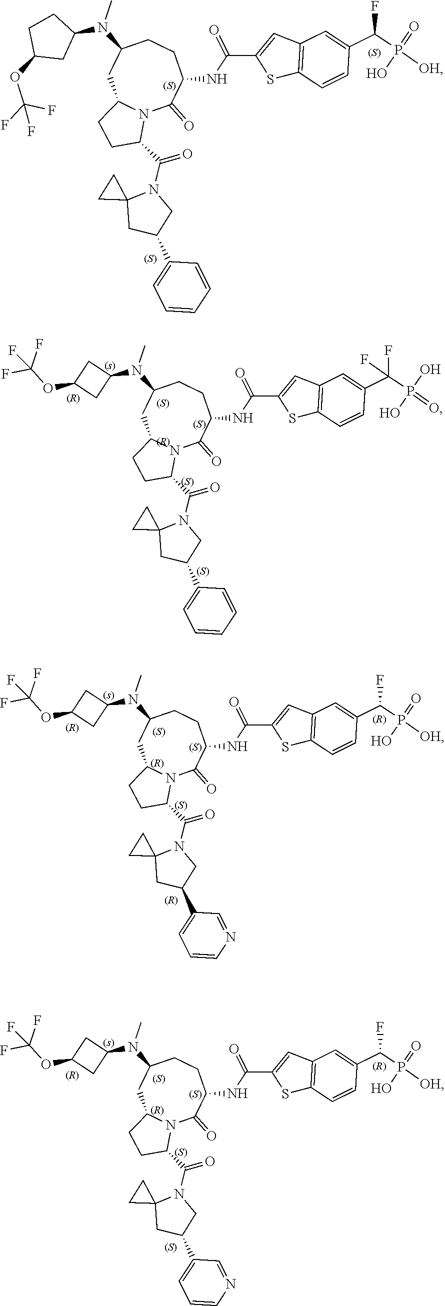 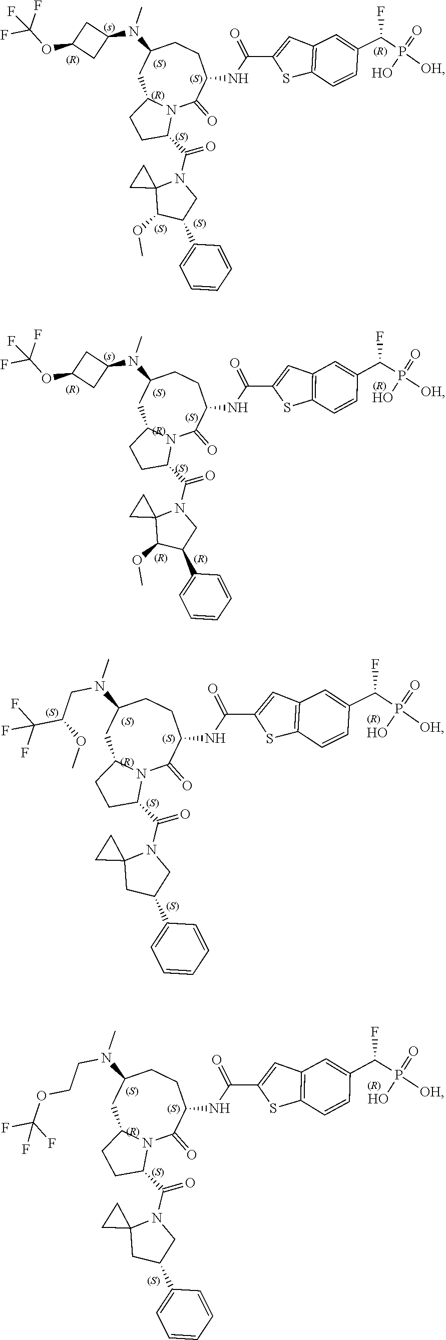 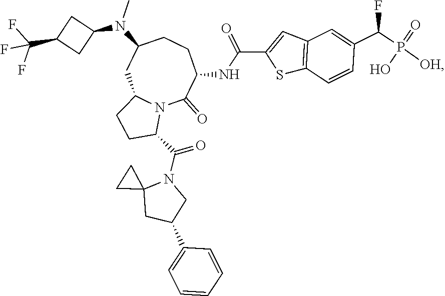 [SMILES: CN([C@H]1CC[C@H](NC(=O)c2cc3cc([C@@H](F)P(=O)(O)O)ccc3s2)C(=O)N2[C@H](CC[C@H]2C(=O)N2C[C@H](c3ccccc3)CC23CC3)C1)[C@H]1C[C@@H](C(F)(F)F)C1]
or a pharmaceutically acceptable salt thereof.

6. The compound of claim 1, or a pharmaceutically acceptable salt thereof, wherein
R^{P1} is —OH and R^{P2} is N-linked amino acid, wherein:
N-linked amino acid is —NR^{Paa1}CR^{Paa2}R^{Paa3}C(O)OH;
R^{Paa1} is H or (C₁-C₆)alkyl; and
R^{Paa2} is H or (C₁-C₆)alkyl; and
R^{Paa3} is H, (C₁-C₆)alkyl, —(C₁-C₆)alkylene-(C₁-C₆)alkoxy;
or —(C₁-C₆)alkylene-(C₆-C₁₀)aryl; or R^{Paa2} and R^{Paa3}, together with the carbon atom to which they are attached form (C₃-C₆)cycloalkyl.

7. The compound of claim 1, wherein the compound is selected from the group consisting of: 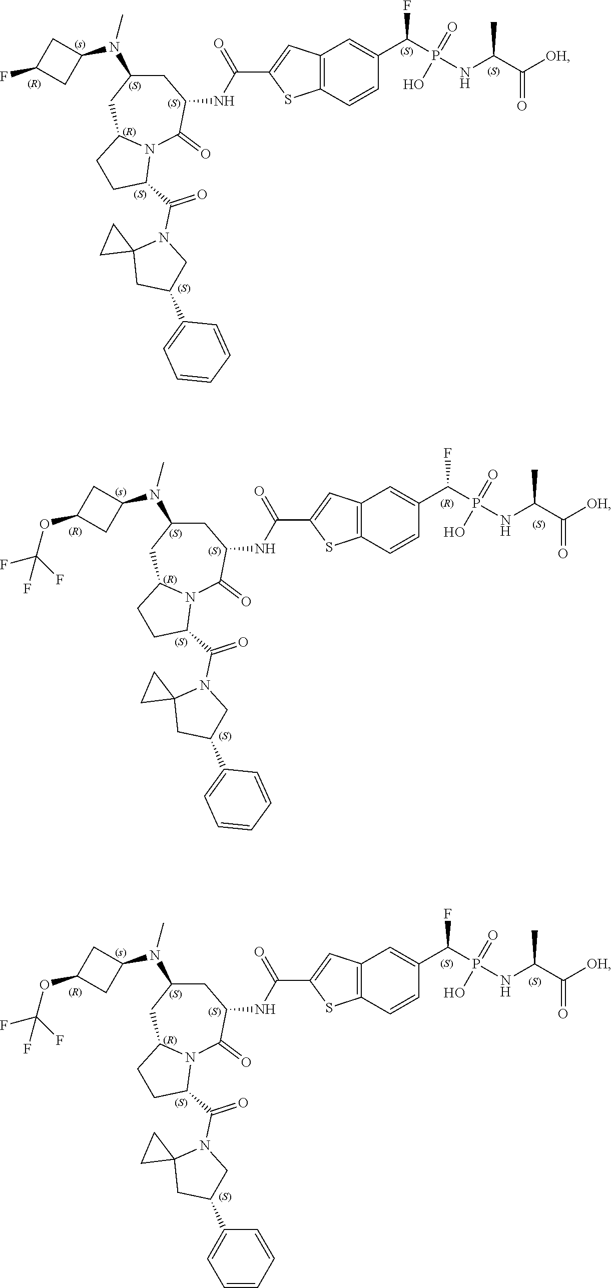 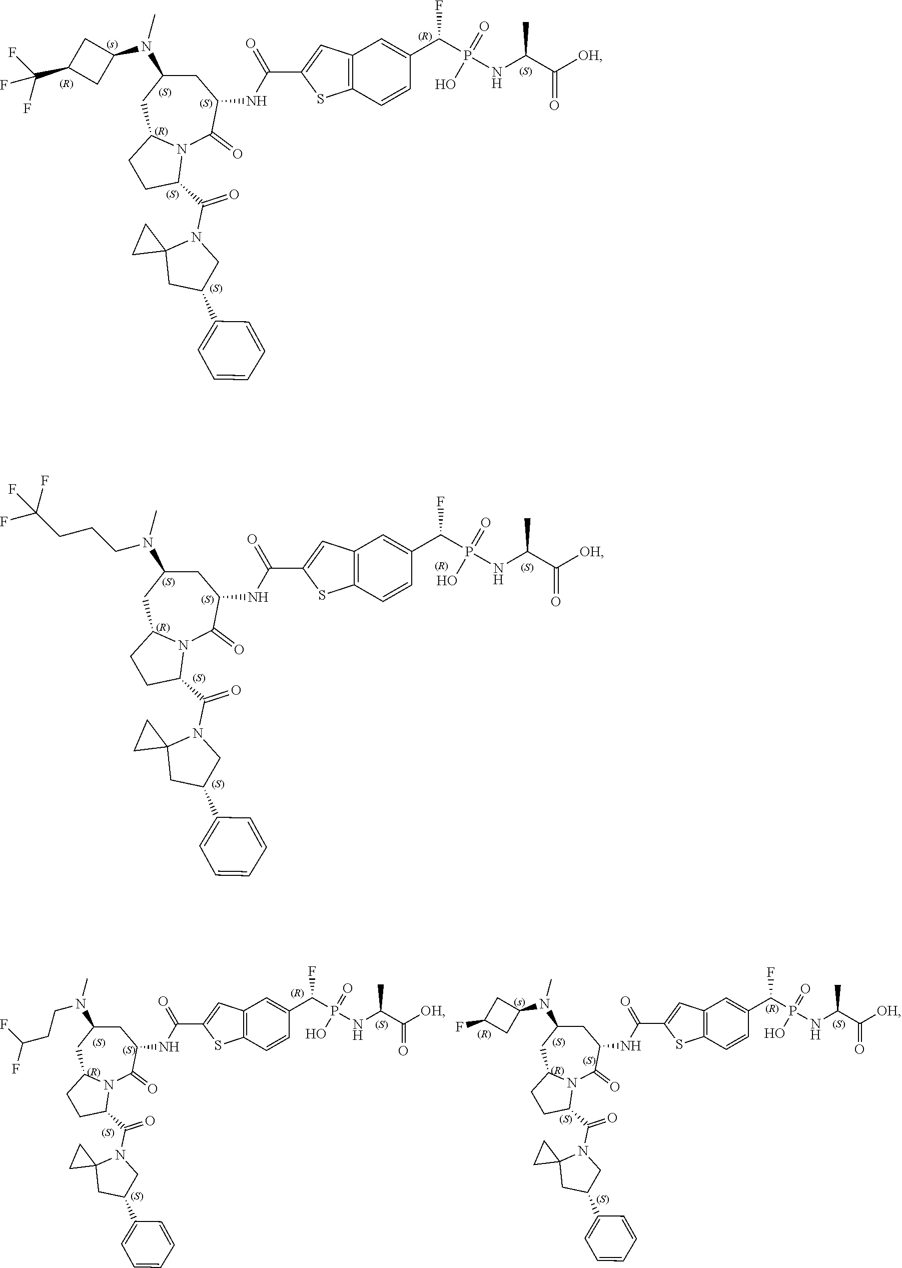 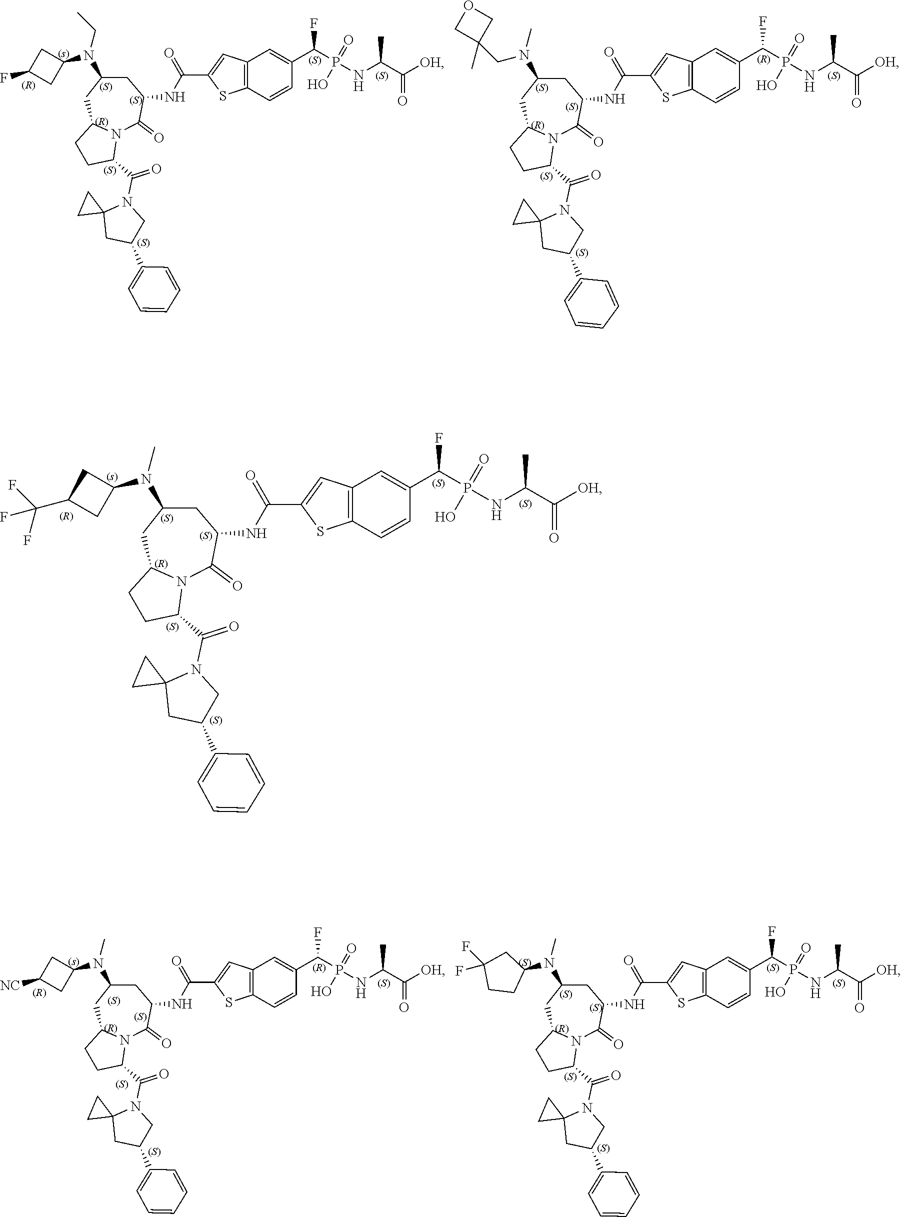 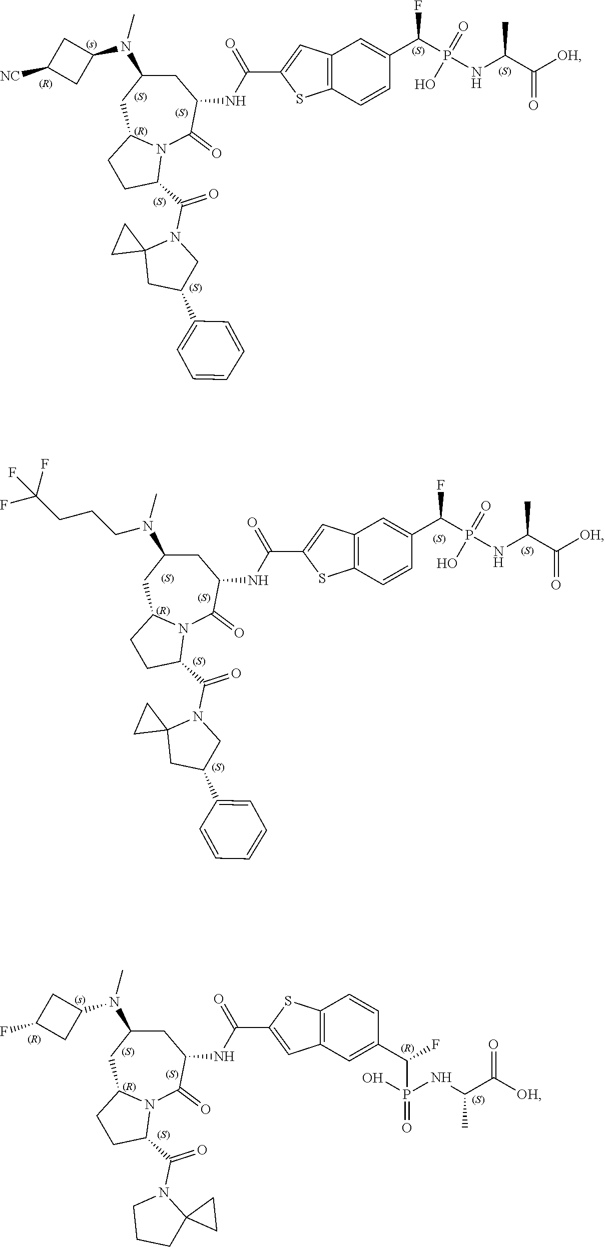 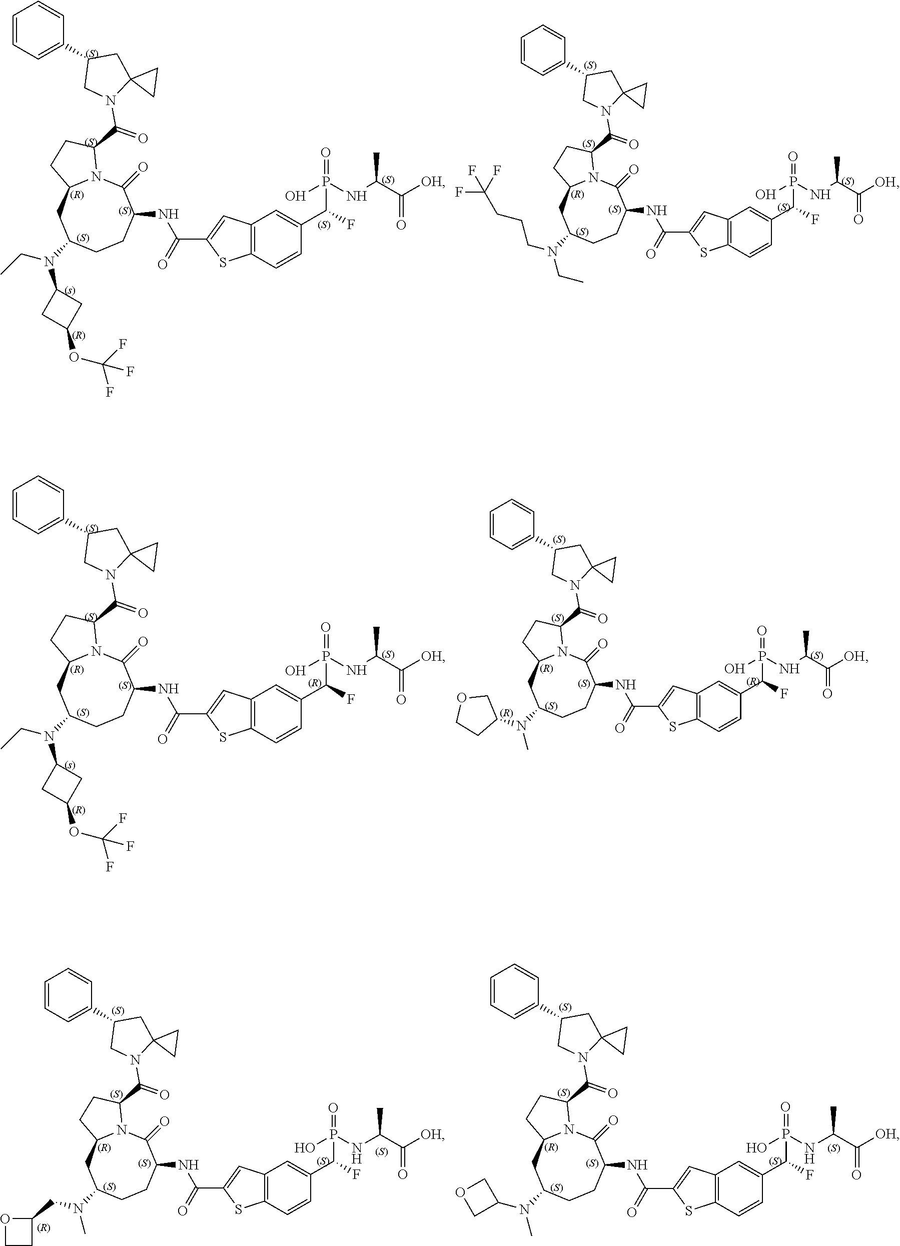 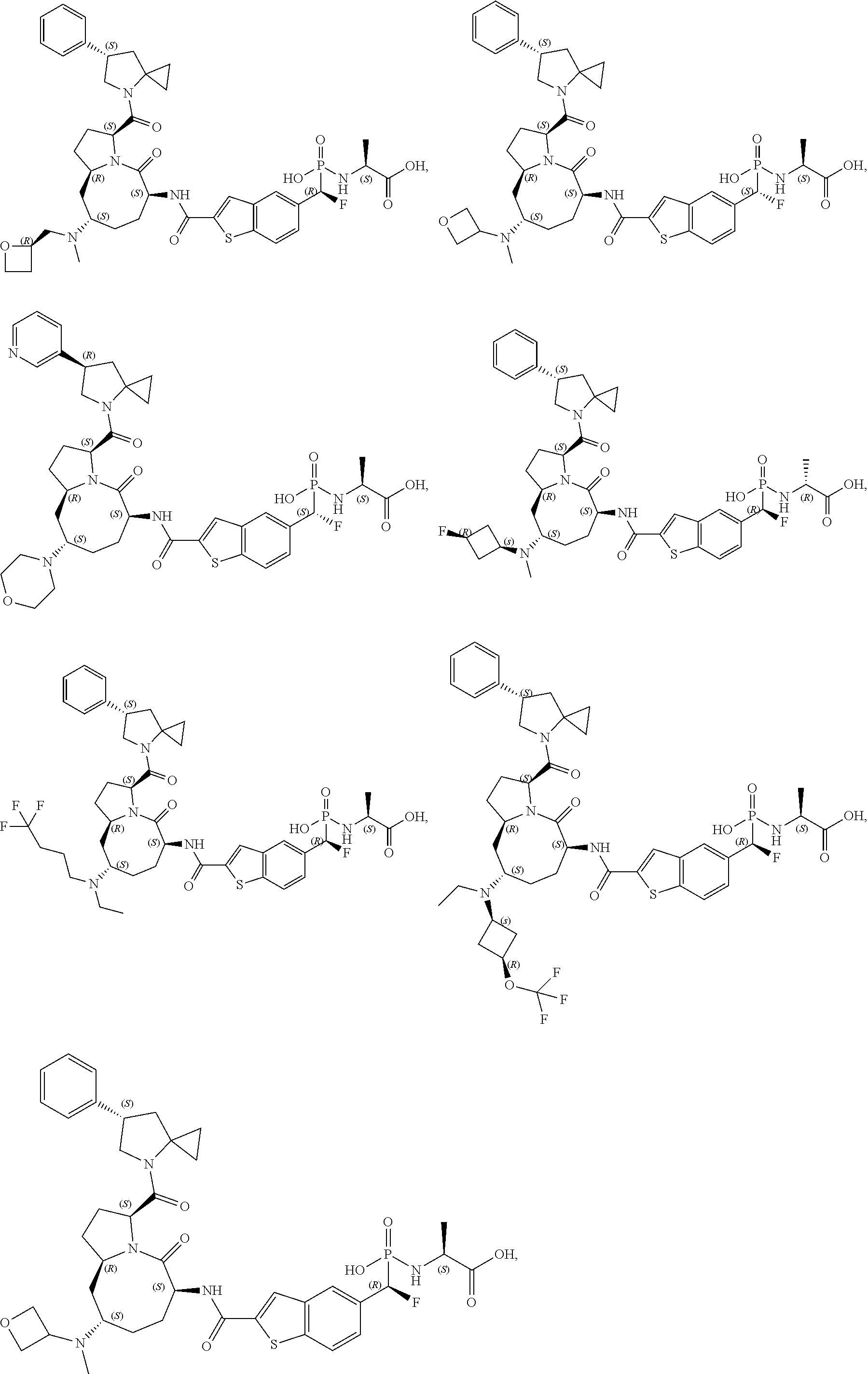
or a pharmaceutically acceptable salt thereof.

8. The compound of claim 1, wherein the compound is of Formula (I-C-3), 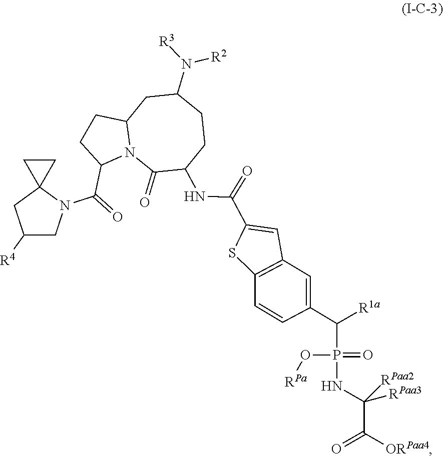
or a pharmaceutically acceptable salt thereof, wherein:
R^{Pa} is (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy;
R^{Paa2} is H or (C₁-C₆)alkyl;
R^{Paa3} is H, (C₁-C₆)alkyl, —(C₁-C₆)alkylene-(C₁-C₆)alkoxy, or —(C₁-C₆)alkylene-(C₆-C₁₀)aryl; or
R^{Paa2} and R^{Paa3}, together with the carbon atom to which they are attached form (C₃-C₆)cycloalkyl; and
R^{Paa4} is (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.

9. The compound of claim 1, wherein the compound is of Formula (I-C-5), 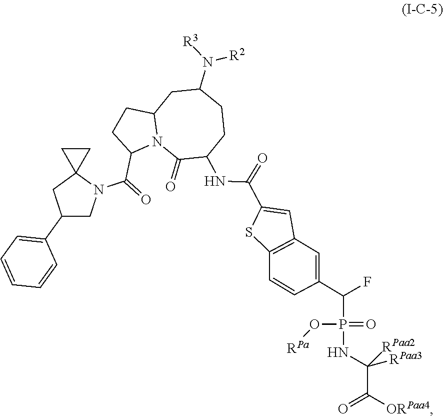
or a pharmaceutically acceptable salt thereof, wherein:
R^{Pa} is (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy;
R^{Paa2} is H or (C₁-C₆)alkyl;
R^{Paa3} is H, (C₁-C₆)alkyl, —(C₁-C₆)alkylene-(C₁-C₆)alkoxy, or —(C₁-C₆)alkylene-(C₆-C₁₀)aryl; or
R^{Paa2} and R^{Pa}3, together with the carbon atom to which they are attached form (C₃-C₆)cycloalkyl; and
R^{Paa4} is (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.

10. The compound of claim 1, wherein the compound is of Formula (I-A-3), 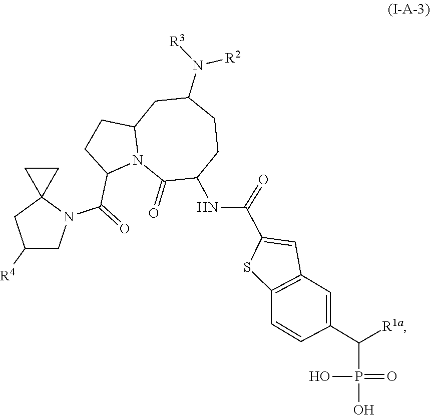
or a pharmaceutically acceptable salt thereof.

11. The compound of claim 1, wherein the compound is of Formula (I-A-5), 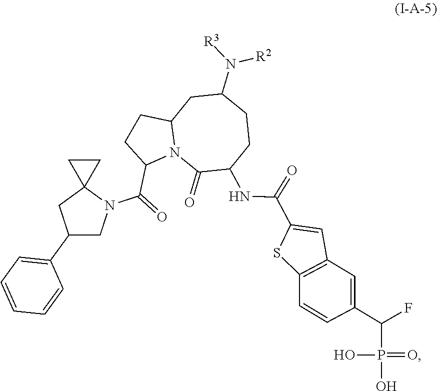
or a pharmaceutically acceptable salt thereof.

12. The compound of claim 1, wherein the compound is of Formula (I-B-3), 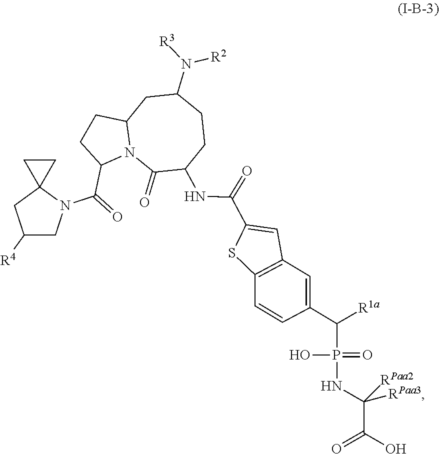
or a pharmaceutically acceptable salt thereof, wherein:
R^{Paa2} is H or (C₁-C₆)alkyl;
R^{Paa3} is H, (C₁-C₆)alkyl, —(C₁-C₆)alkylene-(C₁-C₆)alkoxy, or —(C₁-C₆)alkylene-(C₆-C₁₀)aryl; or
R^{Paa2} and R^{Paa3}, together with the carbon atom to which they are attached form (C₃-C₆)cycloalkyl.

13. The compound of claim 1, wherein the compound is of Formula (I-B-5), 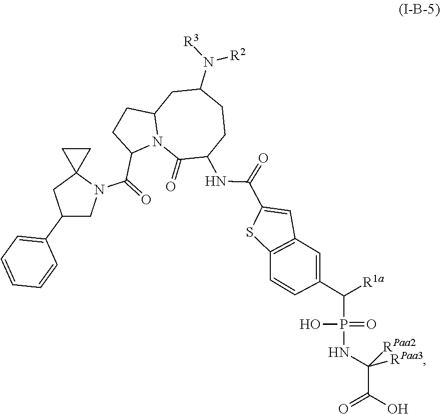
or a pharmaceutically acceptable salt thereof, wherein:
R^{1a} is F;
R^{Paa2} is H or (C₁-C₆)alkyl;
R^{Paa3} is H, (C₁-C₆)alkyl, —(C₁-C₆)alkylene-(C₁-C₆)alkoxy, or —(C₁-C₆)alkylene-(C₆-C₁₀)aryl; or
R^{Paa2} and R^{Paa3}, together with the carbon atom to which they are attached form (C₃-C₆)cycloalkyl.

14. The compound of claim 1, wherein the compound is of Formula (I-H-3), 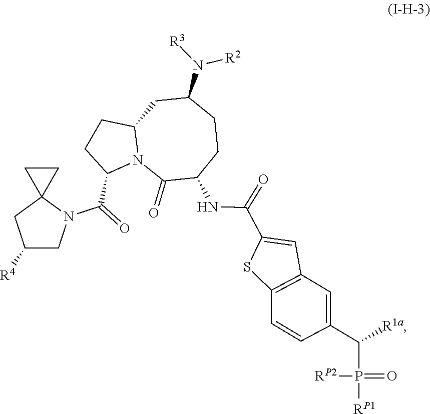
or a pharmaceutically acceptable salt thereof, wherein:
R^{P1} and R^{P2} are each independently —OH, —OR^{Pa}, —N^{Paa1}CR^{Paa2}R^{Paa3}C(O)OH, —NR^{Paa1}CR^{Paa2}R^{Paa3}C(O)OR^{Paa4}, or Ring A; 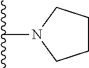 [SMILES: *N1CCCC1]
Ring A is substituted with 0, 1, 2, or 3 substituents each independently selected from the group consisting of halo, oxo, COOH, and —C(O)OR^{PE};
each R^{Pa} is independently (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy;
each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen;
each R^{Paa1} is independently H or (C₁-C₆)alkyl; each R^{Paa2} is independently H or (C₁-C₆)alkyl;
each R^{Paa3} is independently H, (C₁-C₆)alkyl, —(C₁-C₆)alkylene-(C₁-C₆)alkoxy, or —(C₁-C₆)alkylene-(C₆-C₁₀)aryl;
or R^{Paa2} and R^{Paa3}, together with the carbon atom to which they are attached form (C₃-C₆)cycloalkyl;
and each R^{Paa4} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.

15. The compound of claim 14, wherein the compound is of Formula (I-H-5), 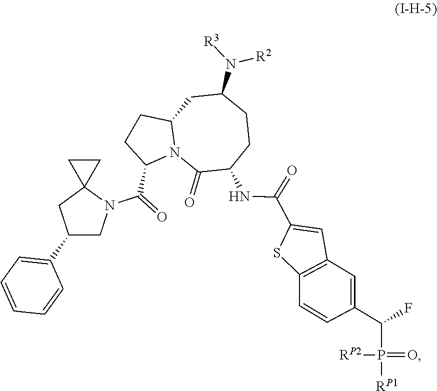
or a pharmaceutically acceptable salt thereof.

16. A pharmaceutical composition comprising the compound of claim 1, or a pharmaceutically acceptable salt thereof, and a pharmaceutically acceptable excipient.

17. A pharmaceutical composition comprising the compound of claim 2, or a pharmaceutically acceptable salt thereof, and a pharmaceutically acceptable excipient.

18. A pharmaceutical composition comprising the compound of claim 3, or a pharmaceutically acceptable salt thereof, and a pharmaceutically acceptable excipient.

19-30. (canceled)

31. The compound of claim 1, wherein the compound is of Formula (I-H-4), 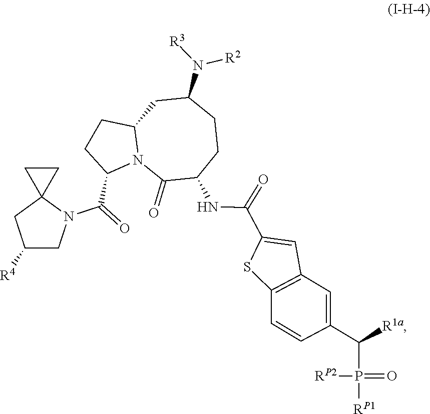
or a pharmaceutically acceptable salt thereof, wherein:
R^{P1} and R^{P2} are each independently —OH, —OR^{Pa}, —NR^{Paa1} CR^{Paa2}R^{Paa3}C(O)OH, —NR^{Paa1}CR^{Paa2}R^{Paa3}C(O)OR^{Paa4}, or Ring A;
Ring A is 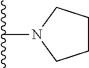 [SMILES: *N1CCCC1]
substituted with 0, 1, 2, or 3 substituents each independently selected from the group consisting of halo, oxo, COOH, and —C(O)OR^{PE};
each R^{Pa} is independently (C₁-C₆)alkyl, halo(C₁-C₆)alkyl, (C₆-C₁₀)aryl, or (C₃-C₆)cycloalkyl, wherein (C₁-C₆)alkyl is substituted with 0, 1, 2, or 3 substituents each independently selected from the group consisting of halo and (C₁-C₆)alkoxy;
each R^{PE} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen;
each R^{Paa1} is independently H or (C₁-C₆)alkyl; each R^{Paa2} is independently H or (C₁-C₆)alkyl;
each R^{Paa3} is independently H, (C₁-C₆)alkyl, —(C₁-C₆)alkylene-(C₁-C₆)alkoxy, or —(C₁-C₆)alkylene-(C₆-C₁₀)aryl;
or R^{Paa2} and R^{Paa3}, together with the carbon atom to which they are attached form (C₃-C₆)cycloalkyl;
and each R^{Paa4} is independently (C₁-C₆)alkyl, halo(C₁-C₆)alkyl, —(C₁-C₆)alkylene-(C₁-C₆)alkoxy, (C₃-C₆)cycloalkyl, (C₅-C₈)spirocycloalkyl, —(C₁-C₆)alkylene-(C₆-C₁₀)aryl, —(C₁-C₆)alkylene-(C₁-C₆)alkoxy, —(C₁-C₆)alkylene-(C₃-C₇)cycloalkyl, 3- to 7-membered heterocyclyl, or —(C₁-C₆)alkylene-(3- to 7-membered heterocyclyl), wherein (C₁-C₆)alkyl, (C₃-C₆)cycloalkyl, (C₅-C₈)spirocycloalkyl, or (C₃-C₇)cycloalkyl of —(C₁-C₆)alkylene-(C₃-C₇)cycloalkyl is each substituted with 0, 1, 2, or 3 independently selected halogen.

32. The compound of claim 31, wherein the compound is of Formula (I-H-6), 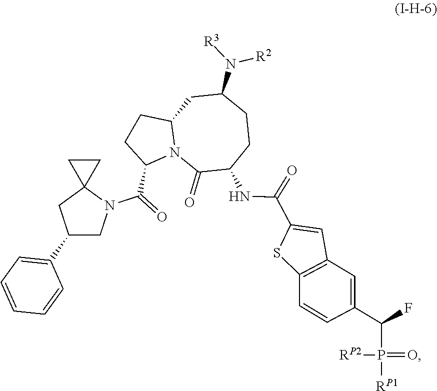
or a pharmaceutically acceptable salt thereof.

33. The compound of claim 2, or a pharmaceutically acceptable salt thereof, wherein R² is (C₁-C₆)alkyl; and R³ is halo(C₁-C₆)alkyl.

34. The compound of claim 4, or a pharmaceutically acceptable salt thereof, wherein R² is (C₁-C₆)alkyl; and R³ is halo(C₁-C₆)alkyl.

35. The compound of claim 2, or a pharmaceutically acceptable salt thereof, wherein R² is (C₁-C₆)alkyl; and R³ is (C₃-C₆)cycloalkyl substituted with 0, 1, 2, or 3 substituents each independently selected from the group consisting of halogen, cyano, halo(C₁-C₆)alkyl, and halo(C₁-C₆)alkoxy.

36. The compound of claim 4, or a pharmaceutically acceptable salt thereof, wherein R² is (C₁-C₆)alkyl; and R³ is (C₃-C₆)cycloalkyl substituted with 0, 1, 2, or 3 substituents each independently selected from the group consisting of halogen, cyano, halo(C₁-C₆)alkyl, and halo(C₁-C₆)alkoxy.

37. The compound of claim 2, wherein 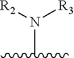
of Formula (I) is

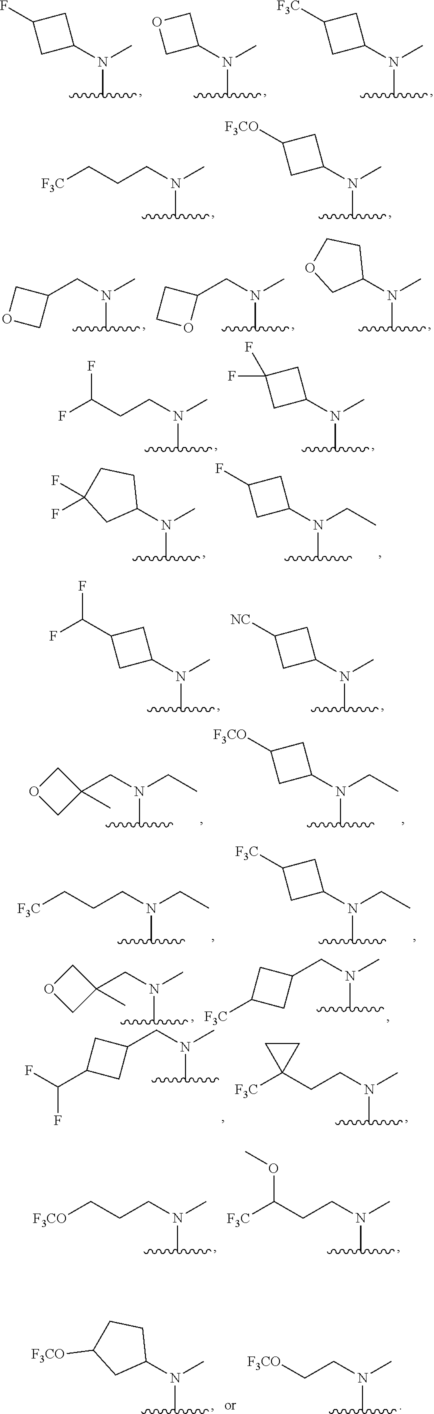

38. The compound of claim 4, wherein 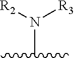
of Formula (I) is

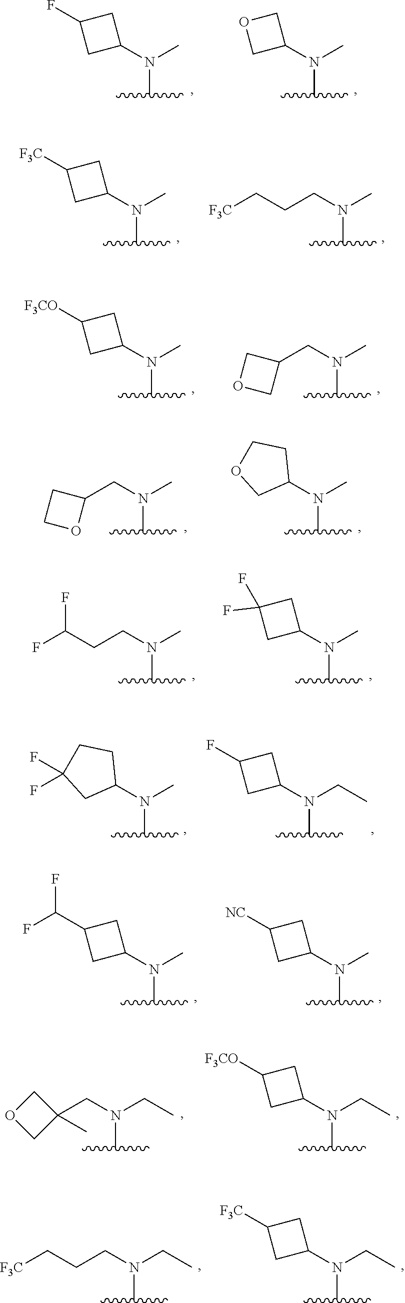

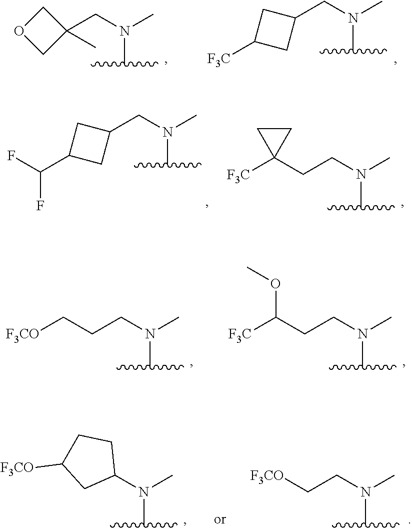

39. The compound of claim 2, or a pharmaceutically acceptable salt thereof, wherein R^{Pa} is phenyl; R^{Paa1} is H; R^{Paa2} is H; R^{Paa3} is (C₁-C₆)alkyl; and R^{Paa4} is (C₁-C₆)alkyl.

40. The compound of claim 2, or a pharmaceutically acceptable salt thereof, wherein R^{Pa} is phenyl; R^{Paa1} is H; R^{Paa2} is H; R^{Paa3} is methyl; and R^{Paa4} is 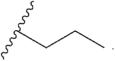 [SMILES: *CCC]
